(19) United States
(12) Patent Application Publication
(10) Pub. No.: US 2021/0170043 A1
(43) Pub. Date: Jun. 10, 2021

(54) DC-SIGN ANTIBODY CONJUGATES COMPRISING STING AGONISTS

(71) Applicants: NOVARTIS AG, Basel (CH); ADURO BIOTECH, INC., Berkley, CA (US)

(72) Inventors: Lisa BARNETT, Poway, CA (US); Steven BENDER, Oceanside, CA (US); Charles Y. CHO, San Diego, CA (US); Sarah COX, San Diego, CA (US); Jonathan Deane, San Diego, CA (US); Scott Martin GLASER, San Diego, CA (US); Xueshi Hao, San Diego, CA (US); Shailaja KASIBHATLA, San Diego, CA (US); Weijia OU, San Diego, CA (US); Tetsuo UNO, San Diego, CA (US); Yongqin WAN, San Diego, CA (US); Ben WEN, Encinitas, CA (US); Tom Yao-Hsiang WU, San Diego, CA (US)

(21) Appl. No.: 16/669,291

(22) Filed: Oct. 30, 2019

Related U.S. Application Data
(60) Provisional application No. 62/753,264, filed on Oct. 31, 2018.

(51) Int. Cl. A61K 47/68; C07K 16/28; A61K 31/688; A61P 35/00

(52) U.S. Cl. CPC A61K 47/6803; A61K 47/6849; A61P 35/00; A61K 31/688; C07K 16/2851

(57) ABSTRACT

Provided herein are immunoconjugates comprising an anti-DC-SIGN antibody conjugated to a STING agonist. Also disclosed are methods of making the immunoconjugates and methods of treating cancer using the immunoconjugates.

### CROSS-REFERENCE TO RELATED APPLICATIONS

This application claims the benefit of U.S. Provisional Application No. 62/753,264 filed Oct. 31, 2018, the content of which are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 11, 2019, is named PAT058304-US-NP_SL.txt and is 536,933 bytes in size.

### FIELD OF THE INVENTION

The present invention generally relates to anti-DC-SIGN antibody conjugates comprising STING agonists, and their uses for the treatment or prevention of cancer.

### BACKGROUND OF THE INVENTION

Dendritic Cell-Specific Intercellular adhesion molecule-3-Grabbing Non-integrin (DC-SIGN) is a C-type lectin receptor present on the surface of both macrophages and dendritic cells (Soilleux E J, et al. (2002) J Luekoc Biol. 71(3):445-57). DC-SIGN recognizes and binds to mannose containing carbohydrates, a class of pathogen-associated molecular patterns (PAMPs) commonly found on viruses, bacteria and fungi. This binding interaction activates phagocytic uptake and internalization of pathogens (McGreal E, et al. (2005) Curr Opin Immunol. 17 (1): 18-24, Engering A, et al. (2002) J Immunol. 168(5):2118-26). Additionally, on myeloid and pre-plasmacytoid dendritic cells, DC-SIGN mediates dendritic cell rolling interactions with blood endothelium and activation of CD4+ T cells (Geijtenbeek T, et al. (2000) Cell 100(5):575-85).

Besides functioning as an adhesion and internalization molecule, recent studies have also shown that DC-SIGN can initiate innate immunity by modulating toll-like receptors (den Dunnen J, et al. (2009) Cancer Immunol. Immunother. 58 (7): 1149-57), though the detailed mechanism is not yet known. Innate immunity is a rapid nonspecific immune response that fights against environmental insults including, but not limited to, pathogens such as bacteria or viruses. Adaptive immunity is a slower but more specific immune response, which confers long-lasting or protective immunity to the host and involves differentiation and activation of naïve T lymphocytes into CD4+T helper cells and/or CD8+ cytotoxic T cells, promoting cellular and humoral immunity. Antigen presentation cells of the innate immune system, such as dendritic cells or macrophages, thus serve as a critical link between the innate and adaptive immune systems by phagocytosing and processing the foreign antigens and presenting them on the cell surface to T cells, thereby activating T cell responses. In cancer biology, DC-SIGN, together with other C-type lectins, is involved in recognition of tumors by dendritic cells and considered to play a critical role in tumor-associated immune responses (van Gisbergen K P et al. (2005) Cancer Res 65(13):5935-44). Additionally, dendritic cells in the tumor microenvironment are often negatively influenced by the surrounding tumor cells and develop a suppressive phenotype (Janco J M et al. (2015) J Immunol. 194(7): 2985-2991). Novel therapies that are able to induce dendritic cell activation represent an important class of potential cancer treatments. Consequently, dendritic cells, and particularly DC-SIGN, are important targets for developing novel cancer immunotherapy treatments.

STING (stimulator of interferon genes) is an intracellular pattern recognition receptor (PRR) associated with the endoplasmic reticulum which acts as a cytosolic DNA sensor (Ishikawa and Barber, Nature 2008, 455(7213):674-678). It was reported that STING comprises four putative transmembrane regions (Ouyang et al., Immunity (2012) 36, 1073), and is able to activate NF-kB, STAT6, and IRF3 transcription pathways to induce expression of type I interferon (e.g., IFN-α and IFN-β) and exert a potent anti-viral state following expression (Ishikawa and Barber, Nature (2008) 455(7213):674-678; Chen et al., Cell (2011) 147, 436-446). In contrast, loss of STING rendered murine embryonic fibroblasts extremely susceptible to negative stranded virus infection, including vesicular stomatitis virus (Ishikawa and Barber, Nature (2008) 455(7213):674-678). Innate immune cells, such a dendritic cells, are potently activated through STING agonism (Woo S R et al. (2014) Immunity 41(5):830-42) and comprise a key responder population to endogenous and pharmacologic STING agonists.

Despite the development of a multitude of effective biologic, small molecule, and more recently cell-based therapeutics for treating cancer, significant clinical challenges, such as tumor heterogeneity, acquired resistance, and subpopulation patient responsiveness remain. There remains an urgent need for new immunotherapies for the treatment of diseases, in particular cancer.

### SUMMARY OF THE INVENTION

The invention is based on the finding that targeting dendritic cells and macrophages, by way of the C-type lectin receptor DC-SIGN, with an antibody conjugated to a STING agonist induces potent dendritic cell and macrophage activation and anti-tumor immune responses. The unique combination of a DC-SIGN targeting agent and a STING agonist, engineered as a single therapeutic agent, may provide greater clinical benefit as compared to combinations of single agents alone.

The invention provides immunoconjugates comprising anti-DC-SIGN antibodies conjugated with STING agonists, pharmaceutically acceptable salts thereof, pharmaceutical compositions thereof and combinations thereof, which are useful for the treatment of diseases, in particular, cancer. The invention further provides methods of treating, preventing, or ameliorating cancer comprising administering to a subject in need thereof an effective amount of an immunoconjugate of the invention. The terms “immunoconjugate” and “antibody conjugate” are used interchangeably herein. The invention also provides compounds comprising STING agonists and a linker which are useful to conjugate to an antibody and thereby make the immunostimmulatory conjugates (or Immune Stimulator Antibody Conjugates (ISACs)) of the invention. Various embodiments of the invention are described herein.

In one embodiment, this application discloses an immunoconjugate comprising an anti-DC-SIGN antibody (Ab), or a functional fragment thereof, coupled to an agonist of Stimulator of Interferon Genes (STING) receptor (D) via a linker (L), wherein the linker optionally comprises one or more cleavage elements.

In one embodiment, the immunoconjugate comprises Formula (I):

Ab-(L-(D)ₘ)ₙ (Formula (I))

wherein:
Ab is an anti-DC-SIGN antibody or a functional fragment thereof;
L is a linker comprising one or more cleavage elements;
D is a drug moiety that has agonist activity against STING receptor;
m is an integer from 1 to 8; and
n is an integer from 1 to 20.

In another embodiment, the immunoconjugate comprises Formula (I):

Ab-(L-(D)ₘ)ₙ (Formula (I))

wherein:
Ab is an anti-DC-SIGN antibody or a functional fragment thereof;
L is a linker;
D is a drug moiety that binds to STING receptor;
m is an integer from 1 to 8; and
n is an integer from 1 to 20;
wherein D, or a cleavage product thereof, that is released from the immunoconjugate has STING agonist activity.

In another embodiment, the immunconjugate comprises Formula (I):

Ab-(L-(D)ₘ)ₙ (Formula (I))

wherein:
Ab is an anti-DC-SIGN antibody or a functional fragment thereof;
L is a linker;
D is a drug moiety that binds to STING receptor;
m is an integer from 1 to 8; and
n is an integer from 1 to 20;
wherein the immunoconjugate delivers D, or a cleavage product thereof, to a cell targeted by the Ab, and wherein D, or the cleavage product thereof, has STING agonist activity.

In another embodiment, the immunoconjugate comprises Formula (I):

Ab-(L-(D)ₘ)ₙ (Formula (I))

wherein:
Ab is an anti-DC-SIGN antibody or a functional fragment thereof;
L is a linker comprising one or more cleavage elements;
D is a drug moiety that binds to STING receptor;
m is an integer from 1 to 8; and
n is an integer from 1 to 20;
wherein the immunoconjugate releases D, or a cleavage product thereof, in a cell targeted by the Ab, and wherein D, or the cleavage product thereof, has STING agonist activity.

In another embodiment, the immunoconjugate comprises Formula (I):

Ab-(L-(D)ₘ)ₙ (Formula (I))

wherein:
Ab is an anti-DC-SIGN antibody or a functional fragment thereof;
L is a linker comprising one or more cleavage elements;
D is a drug moiety that has agonist activity against STING receptor;
m is an integer from 1 to 8; and
n is an integer from 1 to 20;
wherein the immunoconjugate releases D, or a cleavage product thereof, in a cell targeted by the Ab, and wherein D, or the cleavage product thereof, has STING agonist activity in the cell.

In a further embodiment, the present application discloses an immunoconjugate for delivery of a STING receptor agonist to a cell, the immunoconjugate comprising Formula (I):

Ab-(L-(D)ₘ)ₙ (Formula (I))

wherein:
Ab is an anti-DC-SIGN antibody or a functional fragment thereof;
L is a linker comprising one or more cleavage elements;
D is a drug moiety that binds to STING receptor;
m is an integer from 1 to 8; and
n is an integer from 1 to 20;
wherein the immunoconjugate specifically binds to DC-SIGN on the cell surface and is internalized into the cell, and wherein D, or a cleavage product thereof, is cleaved from L and has STING agonist activity as determined by one or more STING agonist assays selected from: an interferon stimulation assay, a hSTING wt assay, a THP1-Dual assay, a TANK binding kinase 1 (TBK1) assay, or an interferon-γ-inducible protein 10 (IP-10) secretion assay.

In some embodiments, D, or the cleavage product thereof, has STING agonist activity if it binds to STING and is able to stimulate production of one or more STING-dependent cytokines in a STING-expressing cell at least 1.1-fold, 1.2-fold, 1.3-fold, 1.4-fold, 1.5-fold, 1.6-fold, 1.7-fold, 1.8-fold, 1.9-fold, 2-fold or greater than an untreated STING-expressing cell. In another embodiment, the STING-dependent cytokine is selected from interferon, type 1 interferon, IFN-α, IFN-β, type 3 interferon, IFNλ, IP10, TNF, IL-6, CXCL9, CCL4, CXCL11, CCL5, CCL3, or CCL8. In other embodiments, D, or the cleavage product thereof, has STING agonist activity if it binds to STING and is able to stimulate phosphorylation of TBK1 in a STING-expressing cell at least 1.1-fold, 1.2-fold, 1.3-fold, 1.4-fold, 1.5-fold, 1.6-fold, 1.7-fold, 1.8-fold, 1.9-fold, 2-fold or greater than an untreated STING-expressing cell. In further embodiments, D, or the cleavage product thereof, has STING agonist activity if it binds to STING and is able to stimulate expression of a STING-dependent transcript selected from any one of the transcripts listed in FIG. 1A-FIG. 10 and FIG. 2A-FIG. 2L in a STING-expressing cell at least 5-fold or greater than the expression level in an untreated STING-expressing cell. In some embodiments, expression of the STING-dependent transcript is increased 5-fold, 6-fold, 7-fold, 8-fold, 9-fold, 10-fold, 11-fold, 12-fold, 13-fold, 14-fold, 15-fold, 20-fold, 30-fold, 40-fold, 50-fold, 60-fold, 70-fold, 70-fold, 80-fold, 90-fold, 100-fold, 200-fold, 300-fold, 400-fold, 500-fold, 700-fold or greater. In another embodiment, D, or the cleavage product thereof, has STING agonist activity if it binds to STING and is able to stimulate expression of a luciferase reporter gene controlled by interferon (IFN)-stimulated response elements in a STING-expressing cell at an EC50 of 20 micromolar (μM), 15 μM, 10 μM, 9 μM, 8 μM, 7 μM, 6 μM, 5 μM, 4 μM, 3 μM, 2 μM, 1 μM, or less. In other embodiments, D, or the cleavage product thereof, has STING agonist activity if it binds to STING and is able to stimulate expression of a luciferase reporter gene controlled by interferon (IFN)-stimulated response elements in a STING-expressing cell to a level equal to or greater than the level of stimulation of 50 μM of 2′3′-cGAMP. In some embodiments, the STING-expressing cell is THP1-Dual cell, and the luciferase reporter gene is the IRF-Lucia reporter gene in THP1-Dual cell, and optionally the STING agonist activity is determined by the THP1-Dual assay described for Table 7. In another embodiment, the luciferase reporter gene is the 5xlSRE-mlFNb-GL4 reporter gene and the STING-expressing cell is a cell expressing wild-type human STING protein, and optionally the STING agonist activity is determined by the hSTING wt assay described in Table 7. In other embodiments, the immunoconjugate stimulates IP-10 secretion from a STING-expressing cell targeted by the Ab at an EC50 of 5 nanomolar (nM) or less in an IP-10 secretion assay.

In some embodiments disclosed herein, the immunoconjugate is parenterally administered. In some embodiments, the Ab specifically binds to human DC-SIGN. In some embodiments, the Ab does not bind to human L-SIGN. In some embodiments, the Ab is human or humanized. In other embodiments, the Ab is a monoclonal antibody.

In some embodiments of the immunconjugate disclosed herein, the Ab comprises a modified Fc region. In one embodiment, the Ab comprises cysteine at one or more of the following positions, which are numbered according to EU numbering:

(a) positions 152, 360 and 375 of the antibody heavy chain, and

(b) positions 107, 159, and 165 of the antibody light chain.

In some embodiments, the anti-DC-SIGN antibody specifically binds to an epitope comprising the amino acid sequence of SEQ ID NOs: 320-323. In some embodiments, the anti-DC-SIGN antibody comprises:
a. a heavy chain variable region that comprises an HCDR1 (Heavy Chain Complementarity Determining Region 1) of SEQ ID NO:1, an HCDR2 (Heavy Chain Complementarity Determining Region 2) of SEQ ID NO:2, and an HCDR3 (Heavy Chain Complementarity Determining Region 3) of SEQ ID NO:3; and a light chain variable region that comprises an LCDR1 (Light Chain Complementarity Determining Region 1) of SEQ ID NO:14, an LCDR2 (Light Chain Complementarity Determining Region 2) of SEQ ID NO:15, and an LCDR3 (Light Chain Complementarity Determining Region 3) of SEQ ID NO:16;
b. a heavy chain variable region that comprises an HCDR1 of SEQ ID NO:25, an HCDR2 of SEQ ID NO:26, and an HCDR3 of SEQ ID NO:27; and a light chain variable region that comprises an LCDR1 of SEQ ID NO:38, an LCDR2 of SEQ ID NO:39, and an LCDR3 of SEQ ID NO:40;
c. a heavy chain variable region that comprises an HCDR1 of SEQ ID NO:49, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:60;
d. a heavy chain variable region that comprises an HCDR1 of SEQ ID NO:74, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:82;
e. a heavy chain variable region that comprises an HCDR1 of SEQ ID NO:88, an HCDR2 of SEQ ID NO:26, and an HCDR3 of SEQ ID NO:50; and a light chain variable region that comprises an LCDR1 of SEQ ID NO:94, an LCDR2 of SEQ ID NO:95, and an LCDR3 of SEQ ID NO:82;
f. a heavy chain variable region that comprises an HCDR1 of SEQ ID NO:111, an HCDR2 of SEQ ID NO:26, and an HCDR3 of SEQ ID NO:27; and a light chain variable region that comprises an LCDR1 of SEQ ID NO:38, an LCDR2 of SEQ ID NO:39, and an LCDR3 of SEQ ID NO:118;
g. a heavy chain variable region that comprises an HCDR1 of SEQ ID NO:49, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:124;
h. a heavy chain variable region that comprises an HCDR1 of SEQ ID NO:74, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:124;
i. a heavy chain variable region that comprises an HCDR1 of SEQ ID NO:88, an HCDR2 of SEQ ID NO:26, and an HCDR3 of SEQ ID NO:50; and a light chain variable region that comprises an LCDR1 of SEQ ID NO:94, an LCDR2 of SEQ ID NO:95, and an LCDR3 of SEQ ID NO:124;
j. a heavy chain variable region that comprises an HCDR1 of SEQ ID NO:138, an HCDR2 of SEQ ID NO:139, and an HCDR3 of SEQ ID NO:140; and a light chain variable region that comprises an LCDR1 of SEQ ID NO:59, an LCDR2 of SEQ ID NO:39, and an LCDR3 of SEQ ID NO:118;
k. a heavy chain variable region that comprises an HCDR1 of SEQ ID NO:153, an HCDR2 of SEQ ID NO:154, and an HCDR3 of SEQ ID NO:155; and a light chain variable region that comprises an LCDR1 of SEQ ID NO:166, an LCDR2 of SEQ ID NO:167, and an LCDR3 of SEQ ID NO:168;
l. a heavy chain variable region that comprises an HCDR1 of SEQ ID NO:178, an HCDR2 of SEQ ID NO:179, and an HCDR3 of SEQ ID NO:180; and a light chain variable region that comprises an LCDR1 of SEQ ID NO:191, an LCDR2 of SEQ ID NO:192, and an LCDR3 of SEQ ID NO:193;
m. a heavy chain variable region that comprises an HCDR1 of SEQ ID NO:203, an HCDR2 of SEQ ID NO:204, and an HCDR3 of SEQ ID NO:205; and a light chain variable region that comprises an LCDR1 of SEQ ID NO:216, an LCDR2 of SEQ ID NO:217, and an LCDR3 of SEQ ID NO:218;
n. a heavy chain variable region that comprises an HCDR1 of SEQ ID NO:227, an HCDR2 of SEQ ID NO:228, and an HCDR3 of SEQ ID NO:229; and a light chain variable region that comprises an LCDR1 of SEQ ID NO:216, an LCDR2 of SEQ ID NO:217, and an LCDR3 of SEQ ID NO:238;
o. a heavy chain variable region that comprises an HCDR1 of SEQ ID NO:244, an HCDR2 of SEQ ID NO:26, and an HCDR3 of SEQ ID NO:245; and a light chain variable region that comprises an LCDR1 of SEQ ID NO:253, an LCDR2 of SEQ ID NO:254, and an LCDR3 of SEQ ID NO:255;
p. a heavy chain variable region that comprises an HCDR1 of SEQ ID NO:264, an HCDR2 of SEQ ID NO:265, and an HCDR3 of SEQ ID NO:266; and a light chain variable region that comprises an LCDR1 of SEQ ID NO:277, an LCDR2 of SEQ ID NO:278, and an LCDR3 of SEQ ID NO:279;
q. a heavy chain variable region that comprises an HCDR1 of SEQ ID NO:264, an HCDR2 of SEQ ID NO:265, and an HCDR3 of SEQ ID NO:296; and a light chain variable region that comprises an LCDR1 of SEQ ID NO:277, an LCDR2 of SEQ ID NO:278, and an LCDR3 of SEQ ID NO:279.

In some embodiments, the anti-DC-SIGN antibody comprises:
a. A heavy chain variable region (VH) comprising the amino acid sequence of SEQ ID NO:10, and a light chain variable region (VL) comprising the amino acid sequence of SEQ ID NO:21;
b. A heavy chain variable region (VH) comprising the amino acid sequence of SEQ ID NO:34, and a light chain variable region (VL) comprising the amino acid sequence of SEQ ID NO:45;
c. A heavy chain variable region (VH) comprising the amino acid sequence of SEQ ID NO:55, and a light chain variable region (VL) comprising the amino acid sequence of SEQ ID NO:64;
d. A heavy chain variable region (VH) comprising the amino acid sequence of SEQ ID NO:34, and a light chain variable region (VL) comprising the amino acid sequence of SEQ ID NO:70;
e. A heavy chain variable region (VH) comprising the amino acid sequence of SEQ ID NO:78, and a light chain variable region (VL) comprising the amino acid sequence of SEQ ID NO:84;
f. A heavy chain variable region (VH) comprising the amino acid sequence of SEQ ID NO:90, and a light chain variable region (VL) comprising the amino acid sequence of SEQ ID NO:99;
g. A heavy chain variable region (VH) comprising the amino acid sequence of SEQ ID NO:103, and a light chain variable region (VL) comprising the amino acid sequence of SEQ ID NO:107;
h. A heavy chain variable region (VH) comprising the amino acid sequence of SEQ ID NO:114, and a light chain variable region (VL) comprising the amino acid sequence of SEQ ID NO:120;
i. A heavy chain variable region (VH) comprising the amino acid sequence of SEQ ID NO:55, and a light chain variable region (VL) comprising the amino acid sequence of SEQ ID NO:126;
j. A heavy chain variable region (VH) comprising the amino acid sequence of SEQ ID NO:78, and a light chain variable region (VL) comprising the amino acid sequence of SEQ ID NO:130;
k. A heavy chain variable region (VH) comprising the amino acid sequence of SEQ ID NO:90, and a light chain variable region (VL) comprising the amino acid sequence of SEQ ID NO:134;
l. A heavy chain variable region (VH) comprising the amino acid sequence of SEQ ID NO:145, and a light chain variable region (VL) comprising the amino acid sequence of SEQ ID NO:149;
m. A heavy chain variable region (VH) comprising the amino acid sequence of SEQ ID NO:162, and a light chain variable region (VL) comprising the amino acid sequence of SEQ ID NO:174;
n. A heavy chain variable region (VH) comprising the amino acid sequence of SEQ ID NO:187, and a light chain variable region (VL) comprising the amino acid sequence of SEQ ID NO:199;
o. A heavy chain variable region (VH) comprising the amino acid sequence of SEQ ID NO:212, and a light chain variable region (VL) comprising the amino acid sequence of SEQ ID NO:223;
p. A heavy chain variable region (VH) comprising the amino acid sequence of SEQ ID NO:234, and a light chain variable region (VL) comprising the amino acid sequence of SEQ ID NO:240;
q. A heavy chain variable region (VH) comprising the amino acid sequence of SEQ ID NO:249, and a light chain variable region (VL) comprising the amino acid sequence of SEQ ID NO:260;
r. A heavy chain variable region (VH) comprising the amino acid sequence of SEQ ID NO:273, and a light chain variable region (VL) comprising the amino acid sequence of SEQ ID NO:284;
s. A heavy chain variable region (VH) comprising the amino acid sequence of SEQ ID NO:288, and a light chain variable region (VL) comprising the amino acid sequence of SEQ ID NO:292; or
t. A heavy chain variable region (VH) comprising the amino acid sequence of SEQ ID NO:298, and a light chain variable region (VL) comprising the amino acid sequence of SEQ ID NO:284.

In some embodiments, the anti-DC-SIGN antibody comprises:
a. A heavy chain comprising the amino acid sequence of SEQ ID NO:12, and a light chain comprising the amino acid sequence of SEQ ID NO:23;
b. A heavy chain comprising the amino acid sequence of SEQ ID NO:36, and a light chain comprising the amino acid sequence of SEQ ID NO:47;
c. A heavy chain comprising the amino acid sequence of SEQ ID NO:57, and a light chain comprising the amino acid sequence of SEQ ID NO:66;
d. A heavy chain comprising the amino acid sequence of SEQ ID NO:36, and a light chain comprising the amino acid sequence of SEQ ID NO:72;
e. A heavy chain comprising the amino acid sequence of SEQ ID NO:80, and a light chain comprising the amino acid sequence of SEQ ID NO:86;
f. A heavy chain comprising the amino acid sequence of SEQ ID NO:92, and a light chain comprising the amino acid sequence of SEQ ID NO:101;
g. A heavy chain comprising the amino acid sequence of SEQ ID NO:105, and a light chain comprising the amino acid sequence of SEQ ID NO:109;
h. A heavy chain comprising the amino acid sequence of SEQ ID NO:116, and a light chain comprising the amino acid sequence of SEQ ID NO:122;
i. A heavy chain comprising the amino acid sequence of SEQ ID NO:57, and a light chain comprising the amino acid sequence of SEQ ID NO:128;
j. A heavy chain comprising the amino acid sequence of SEQ ID NO:80, and a light chain comprising the amino acid sequence of SEQ ID NO:132;
k. A heavy chain comprising the amino acid sequence of SEQ ID NO:92, and a light chain comprising the amino acid sequence of SEQ ID NO:136;
l. A heavy chain comprising the amino acid sequence of SEQ ID NO:147, and a light chain comprising the amino acid sequence of SEQ ID NO:151;
m. A heavy chain comprising the amino acid sequence of SEQ ID NO:164, and a light chain comprising the amino acid sequence of SEQ ID NO:176;
n. A heavy chain comprising the amino acid sequence of SEQ ID NO:189, and a light chain comprising the amino acid sequence of SEQ ID NO:201;
o. A heavy chain comprising the amino acid sequence of SEQ ID NO:214, and a light chain comprising the amino acid sequence of SEQ ID NO:225;
p. A heavy chain comprising the amino acid sequence of SEQ ID NO:236, and a light chain comprising the amino acid sequence of SEQ ID NO:242;
q. A heavy chain comprising the amino acid sequence of SEQ ID NO:251, and a light chain comprising the amino acid sequence of SEQ ID NO:262;
r. A heavy chain) comprising the amino acid sequence of SEQ ID NO:275, and a light chain comprising the amino acid sequence of SEQ ID NO:286;
s. A heavy chain comprising the amino acid sequence of SEQ ID NO:290, and a light chain comprising the amino acid sequence of SEQ ID NO:294; or
t. A heavy chain comprising the amino acid sequence of SEQ ID NO:300, and a light chain comprising the amino acid sequence of SEQ ID NO:286.

In some embodiments, L is attached to the Ab via conjugation to one or more modified cysteine residues in the Ab. In one embodiment, L is conjugated to the Ab via modified cysteine residues at positions 152 and 375 of the heavy chain of the Ab, wherein the positions are determined according to EU numbering. In one embodiment, L is conjugated to the Ab via modified cysteine residue at position 152 of the heavy chain of the Ab, wherein the position is determined according to EU numbering. In one embodiment, L is conjugated to the Ab via modified cysteine residue at position 375 of the heavy chain of the Ab, wherein the position is determined according to EU numbering. In some embodiments, L is conjugated via a maleimide linkage to the cysteine.

In one embodiment of the immunoconjugates disclosed herein, D is a dinucleotide. In some cases, D is a cyclic dinucleotide (CDN). In other embodiments, D is a compound selected from any one of the compounds of Table 1, Table 2, Table 3, or Table 4.

In some embodiments disclosed herein, D is a compound selected from

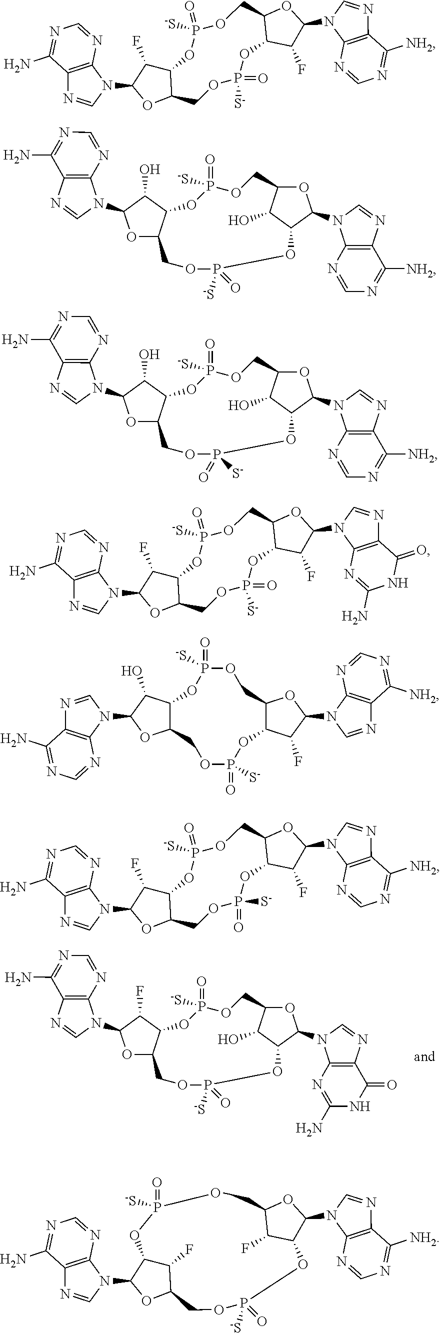

In some embodiments disclosed herein, D is a compound selected from

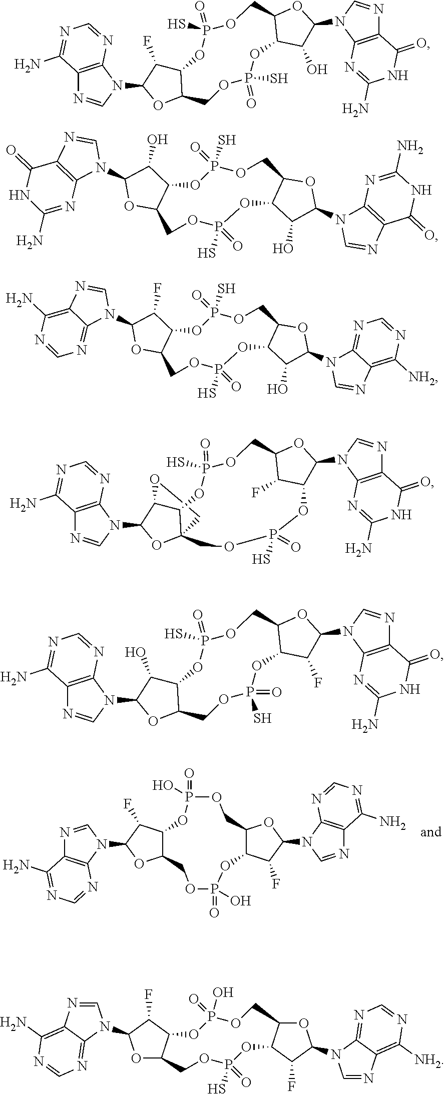

In some embodiments disclosed herein, D is a compound selected from

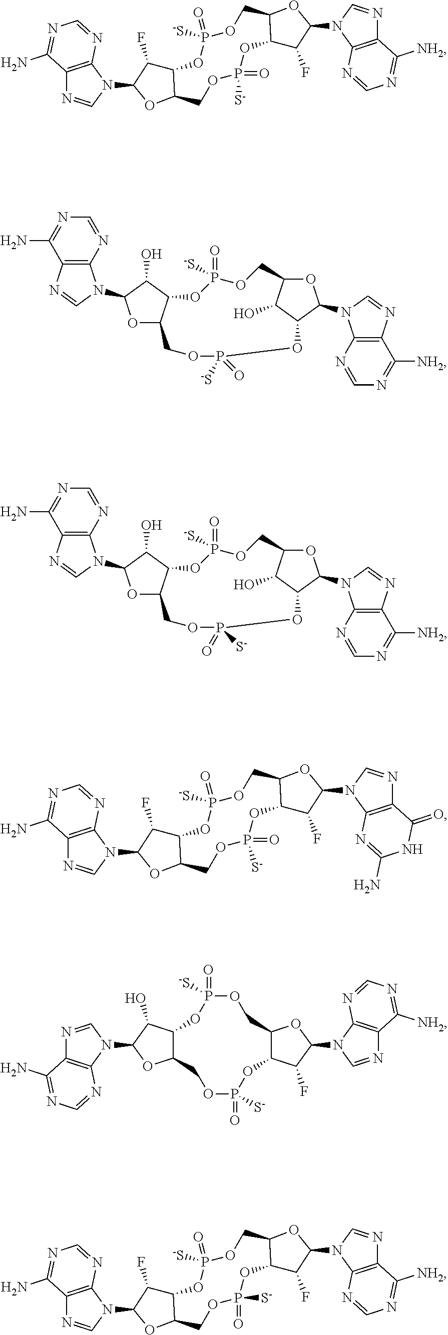

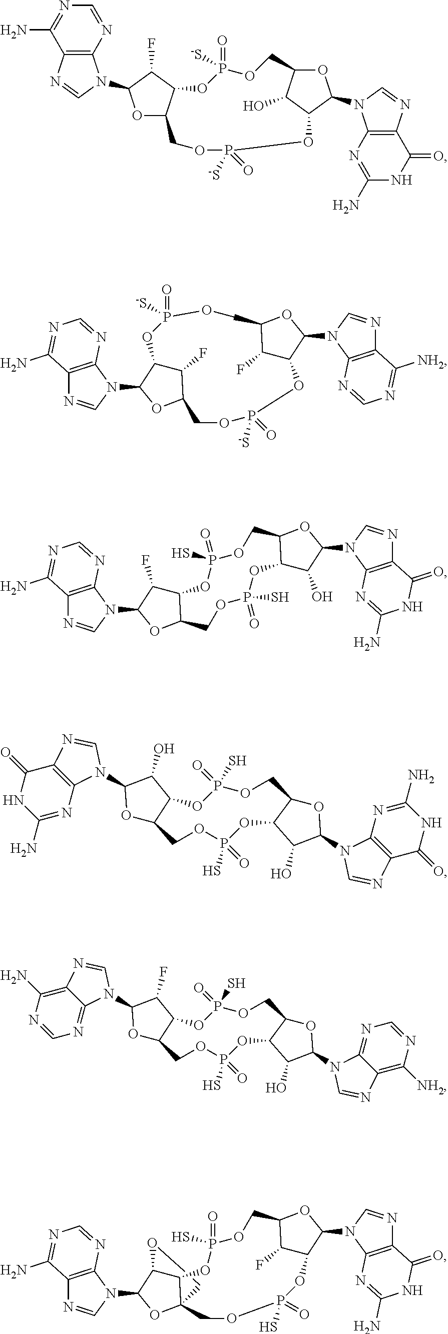

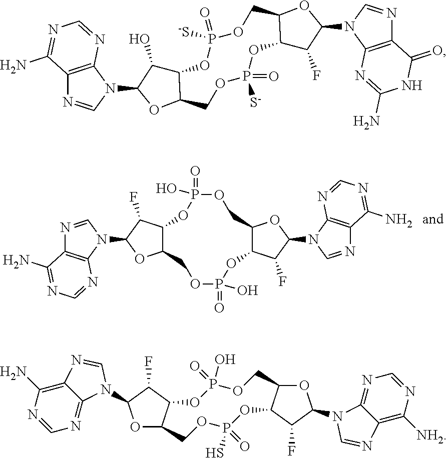

In one embodiment, the present application discloses immunoconjugates wherein L is a cleavable linker comprising one or more cleavage elements. In some embodiments, L comprises two or more cleavage elements, and each cleavage element is independently selected from a self-immolative spacer and a group that is susceptible to cleavage. In some embodiments, the cleavage is selected from acid-induced cleavage, peptidase-induced cleavage, esterase-induced cleavage, glycosidase-induced cleavage, phosphodiesterase-induced cleavage, phosphatase-induced cleavage, protease-induced cleavage, lipase-induced cleavage, or disulfide bond cleavage.

In one embodiment of the immunconjugates disclosed herein the Linker-Drug Moiety (-(L-(D)ₘ)), wherein m is 1, has a structure selected from:

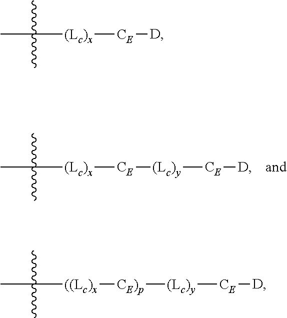

wherein:
Lc is a linker component and each Lc is independently selected from a linker component as disclosed herein;
x is an integer selected from 1, 2, 3, 4, 5, 6, 7, 8, 9, 10, 11, 12, 13, 14, 15, 16, 17, 18, 19 and 20;
y is an integer selected from 1, 2, 3, 4, 5, 6, 7, 8, 9, 10, 11, 12, 13, 14, 15, 16, 17, 18, 19 and 20;
p is an integer selected from 1, 2, 3, 4, 5, 6, 7, 8, 9 and 10;
and each cleavage element (C_{E}) is independently selected from a self-immolative spacer and a group that is susceptible to cleavage selected from acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

In one embodiment of the immunconjugates disclosed herein the Linker (L) of the Linker-Drug Moiety (-(L-(D)ₘ)), wherein m is 1, has a structure selected from:

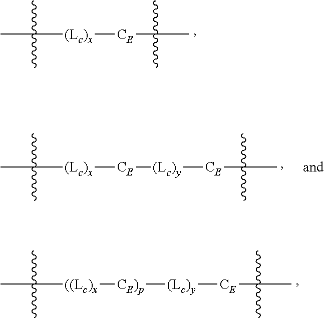

wherein:
Lc is a linker component and each Lc is independently selected from a linker component as disclosed herein;
x is an integer selected from 1, 2, 3, 4, 5, 6, 7, 8, 9, 10, 11, 12, 13, 14, 15, 16, 17, 18, 19 and 20;
y is an integer selected from 1, 2, 3, 4, 5, 6, 7, 8, 9, 10, 11, 12, 13, 14, 15, 16, 17, 18, 19 and 20;
p is an integer selected from 1, 2, 3, 4, 5, 6, 7, 8, 9 and 10;
and each cleavage element (C_{E}) is independently selected from a self-immolative spacer and a group that is susceptible to cleavage selected from acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage. In some embodiments, L has a structure selected from the following, or L comprises a structural component selected from the following:

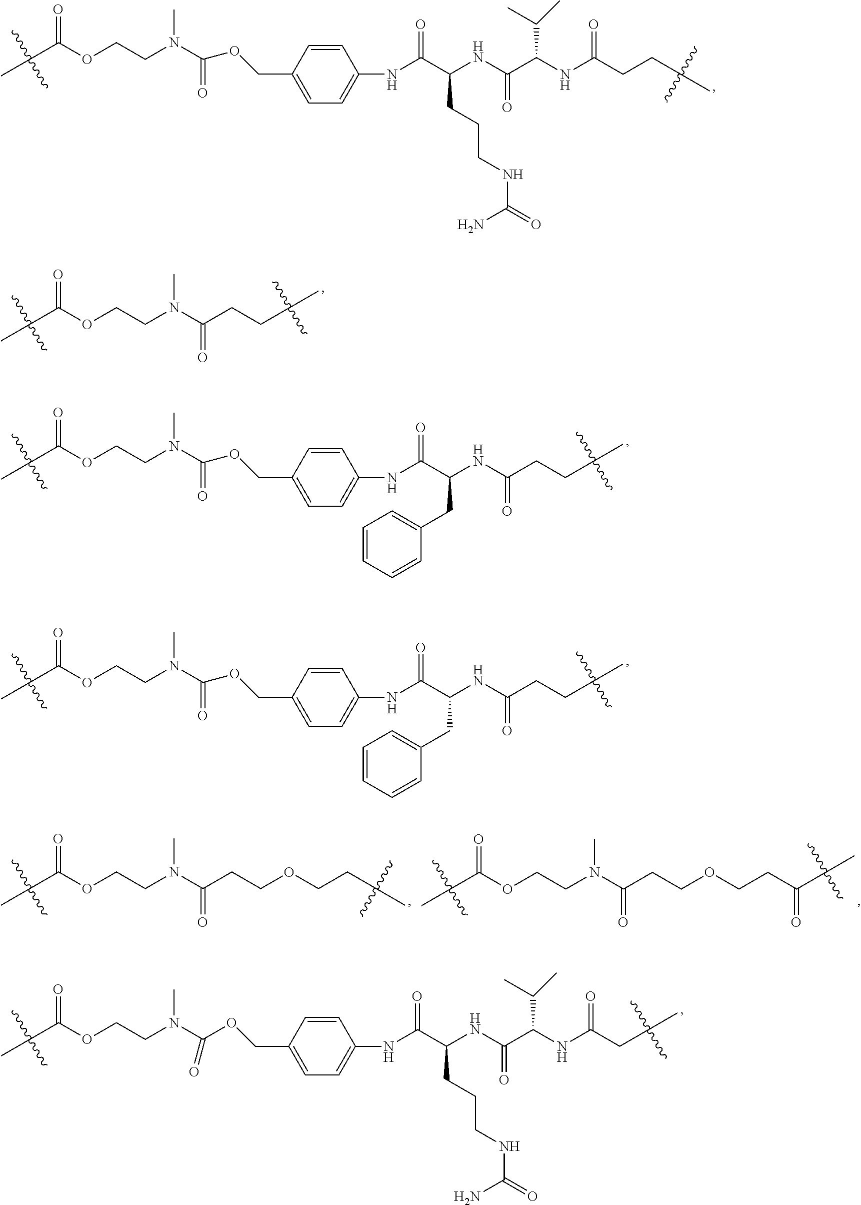

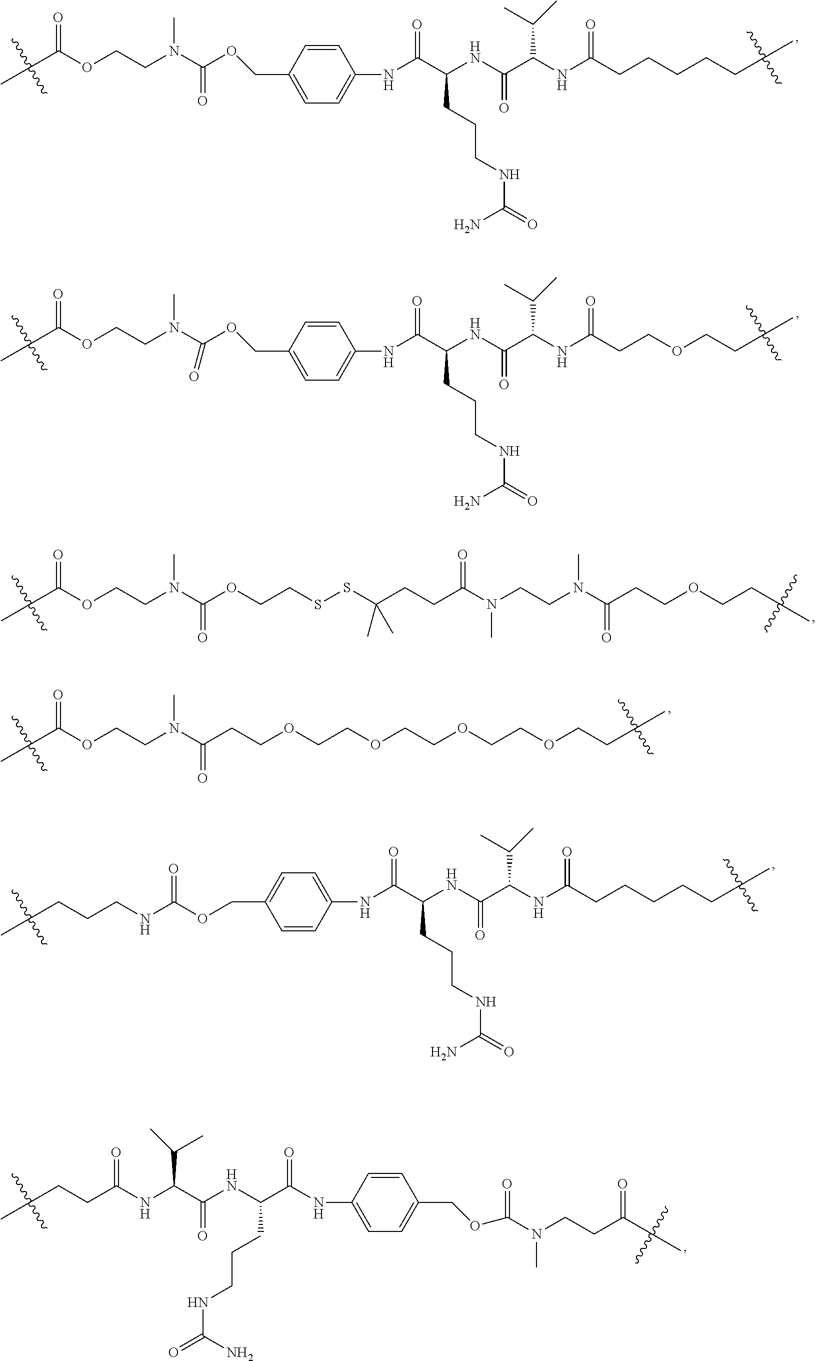

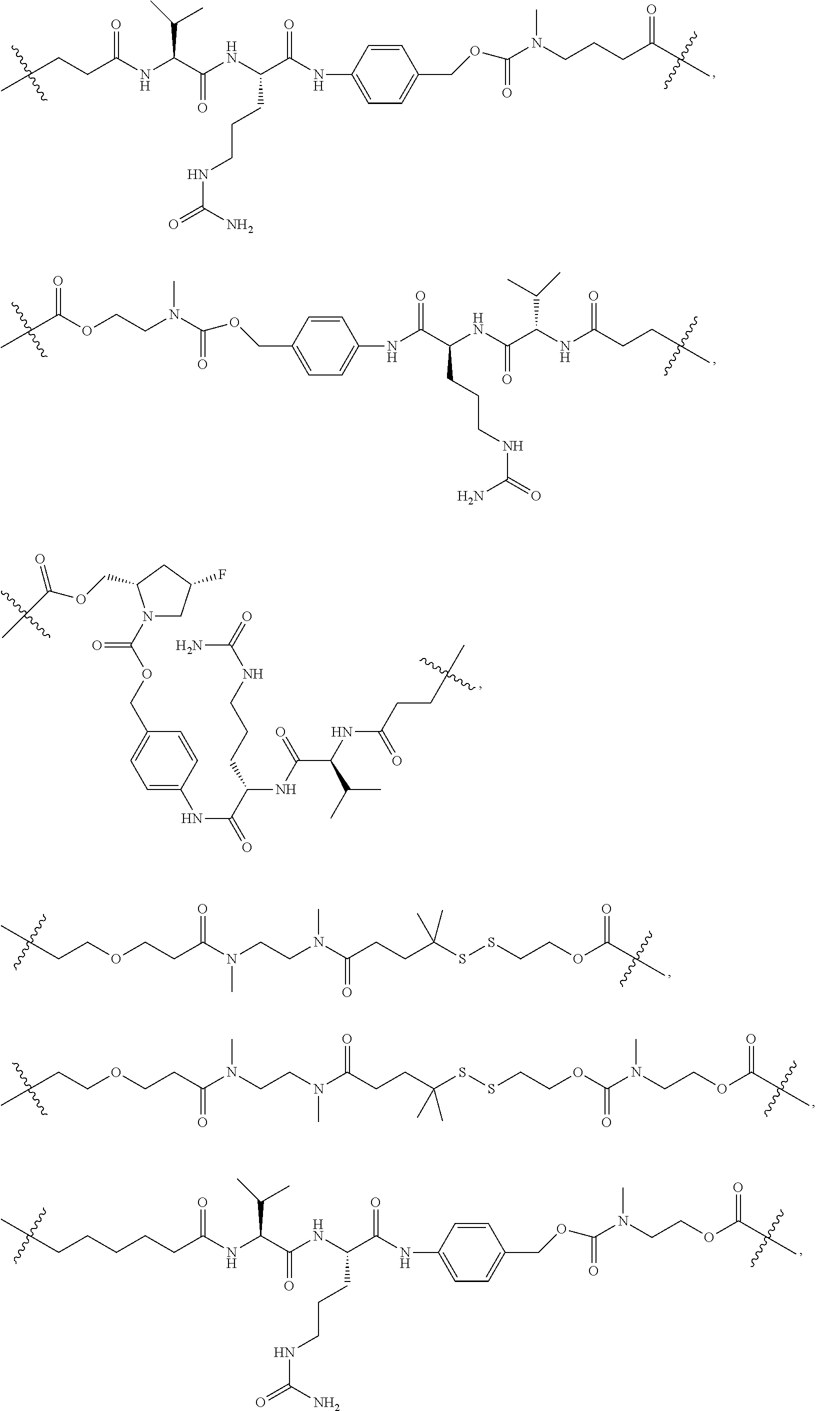

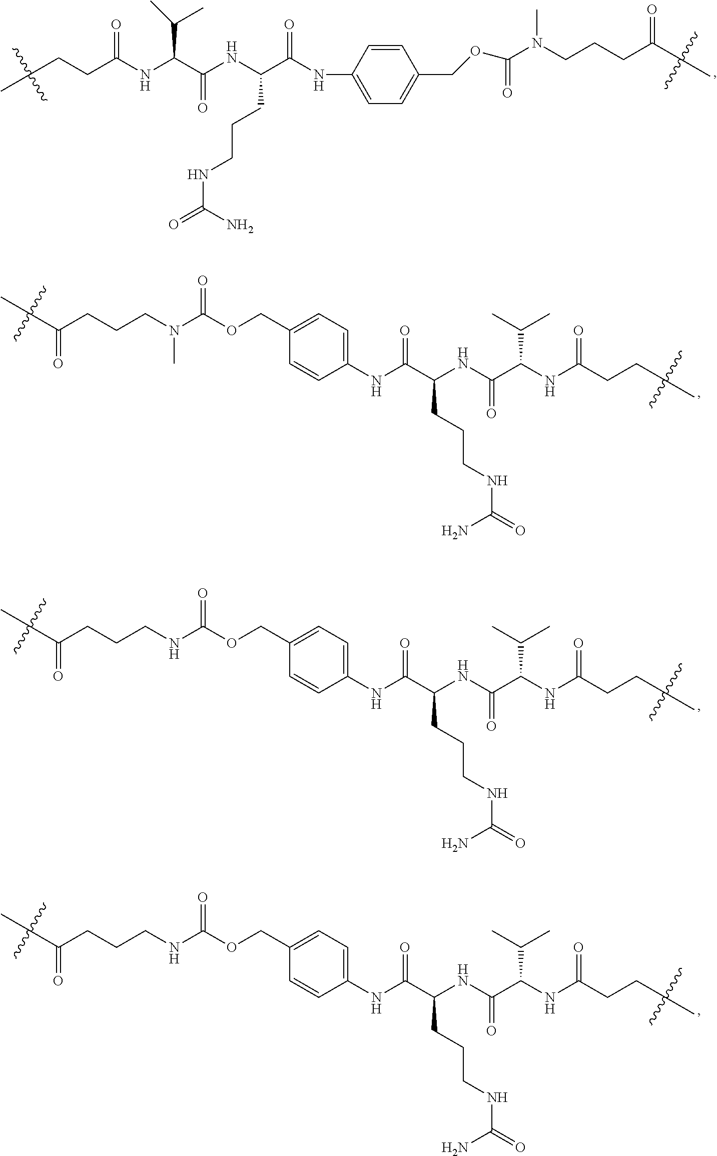 [SMILES: *CCC(=O)N[C@H](C(=O)N[C@@H](CCCNC(N)=O)C(=O)Nc1ccc(COC(=O)N(C)CCCC(*)=O)cc1)C(C)C ; *CCC(=O)N[C@H](C(=O)N[C@@H](CCCNC(N)=O)C(=O)Nc1ccc(COC(=O)N(C)CCCC(*)=O)cc1)C(C)C ; *CCC(=O)N[C@H](C(=O)N[C@@H](CCCNC(N)=O)C(=O)Nc1ccc(COC(=O)NCCCC(*)=O)cc1)C(C)C ; *CCC(=O)N[C@H](C(=O)N[C@@H](CCCNC(N)=O)C(=O)Nc1ccc(COC(=O)NCCCC(*)=O)cc1)C(C)C]

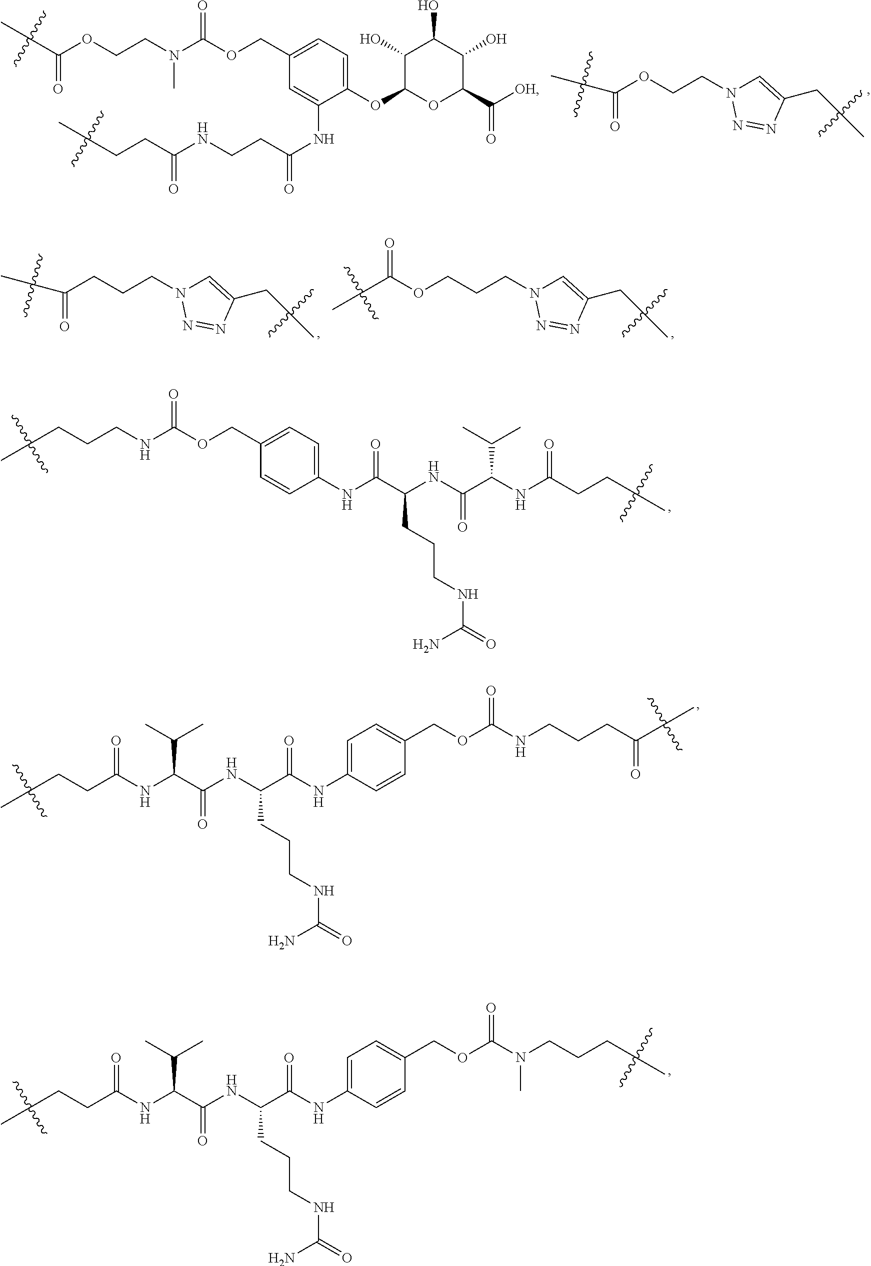

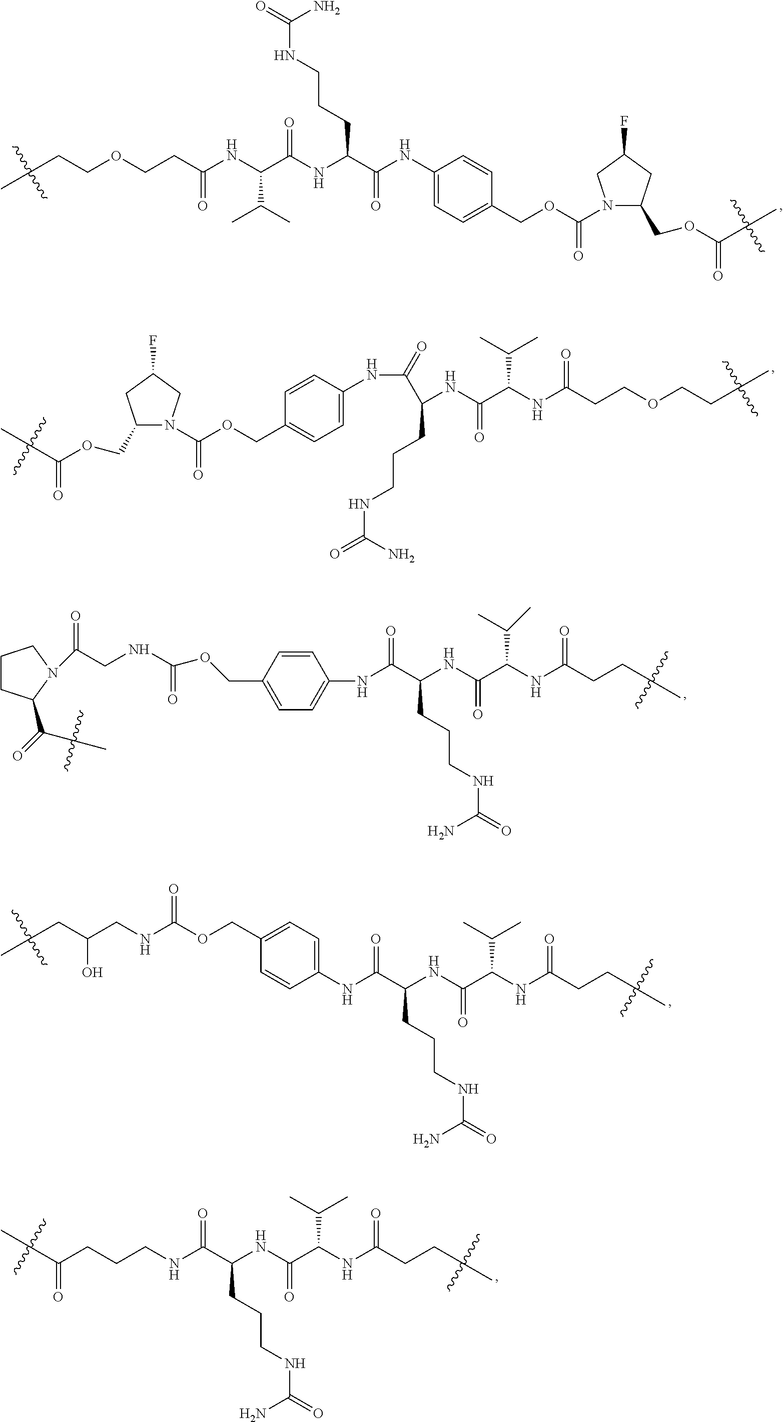 [SMILES: *CCOCCC(=O)N[C@H](C(=O)N[C@@H](CCCNC(N)=O)C(=O)Nc1ccc(COC(=O)N2C[C@@H](F)C[C@H]2COC(*)=O)cc1)C(C)C ; *CCOCCC(=O)N[C@H](C(=O)N[C@@H](CCCNC(N)=O)C(=O)Nc1ccc(COC(=O)N2C[C@@H](F)C[C@H]2COC(*)=O)cc1)C(C)C ; *CCC(=O)N[C@H](C(=O)N[C@@H](CCCNC(N)=O)C(=O)Nc1ccc(COC(=O)NCC(=O)N2CCC[C@@H]2C(*)=O)cc1)C(C)C ; *CCC(=O)N[C@H](C(=O)N[C@@H](CCCNC(N)=O)C(=O)Nc1ccc(COC(=O)NCC(O)C*)cc1)C(C)C ; *CCC(=O)N[C@H](C(=O)N[C@@H](CCCNC(N)=O)C(=O)NCCCC(*)=O)C(C)C]

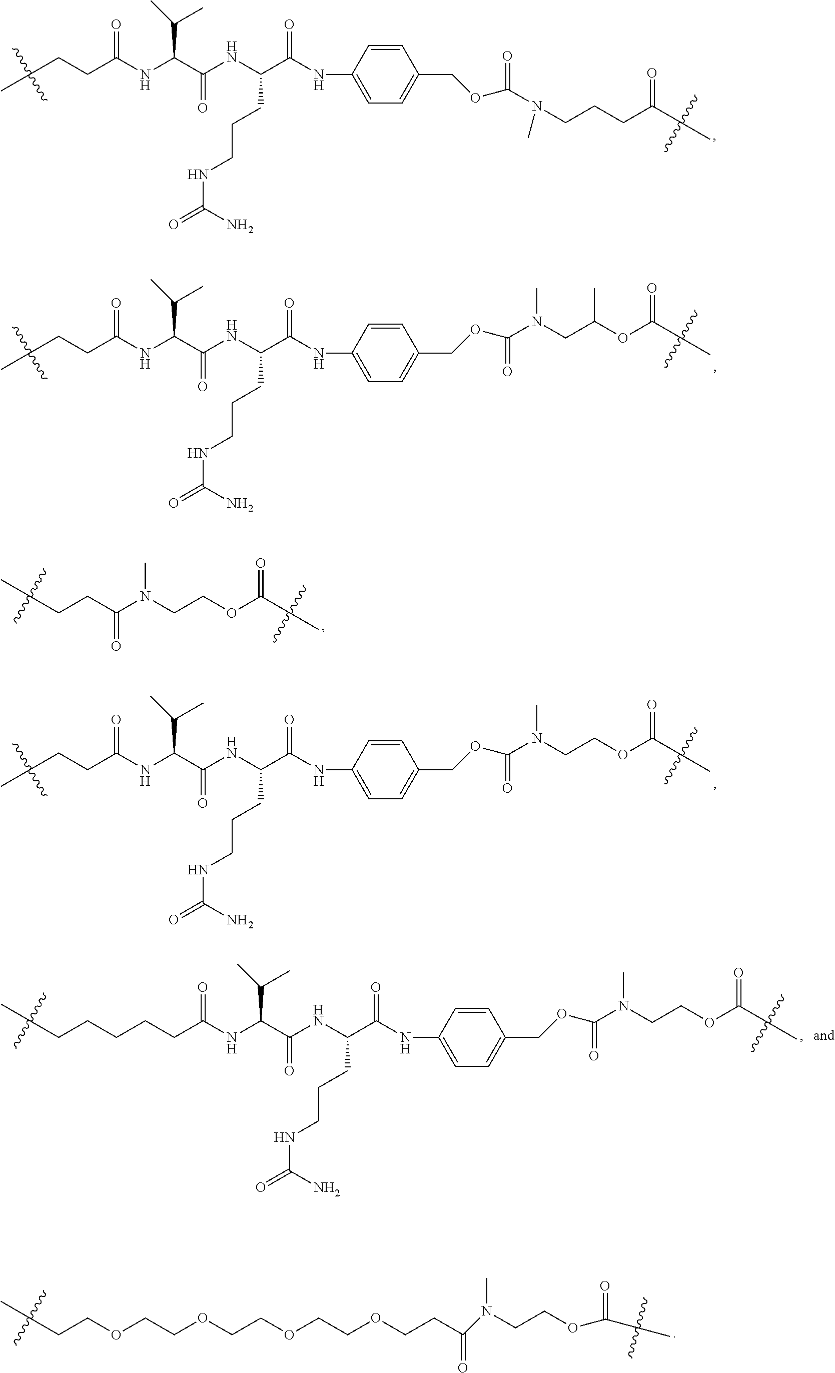

In some embodiments disclosed herein, the immunoconjugate is selected from the following:

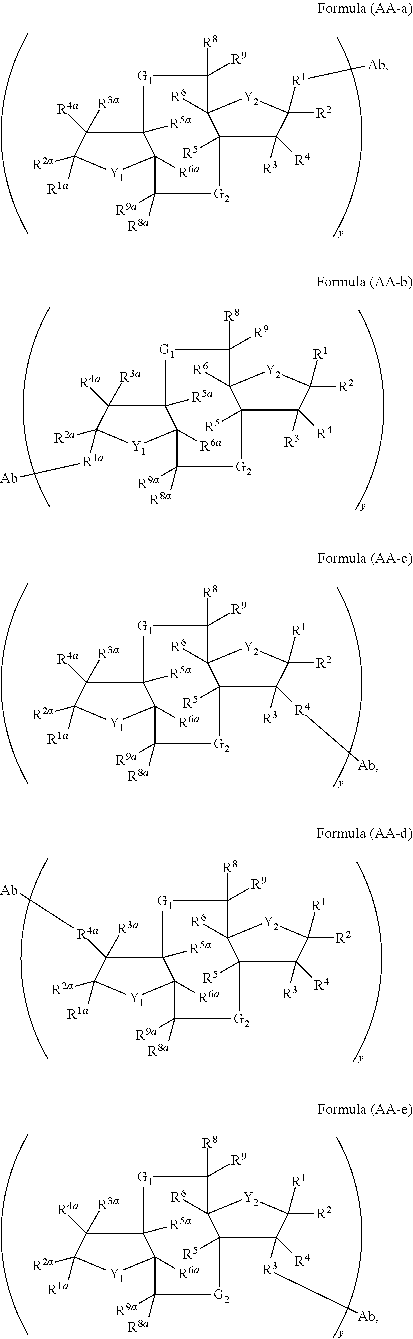

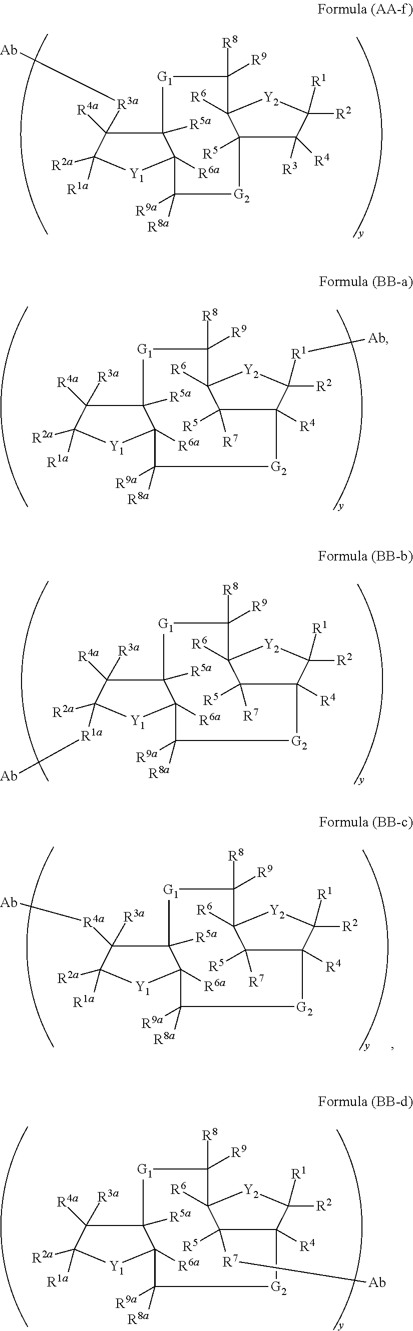

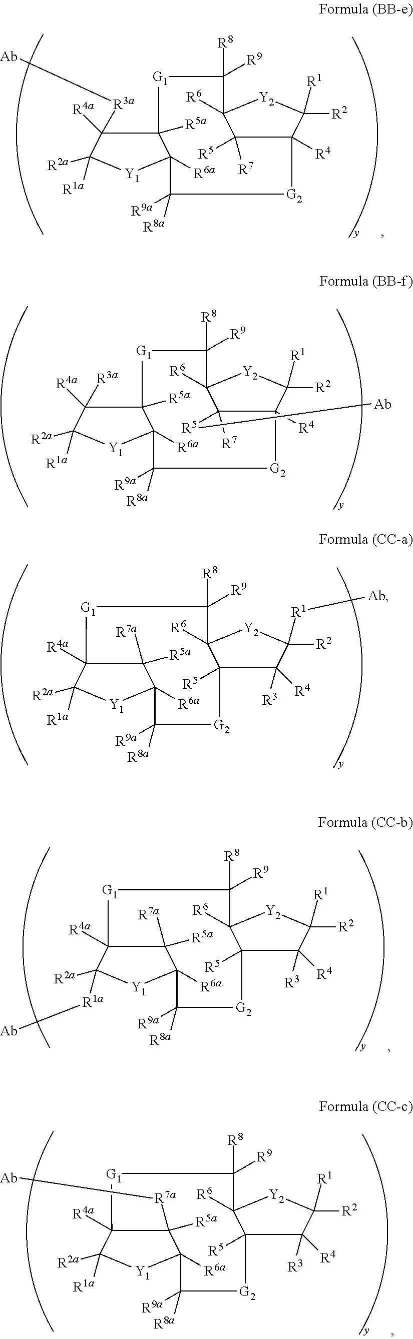

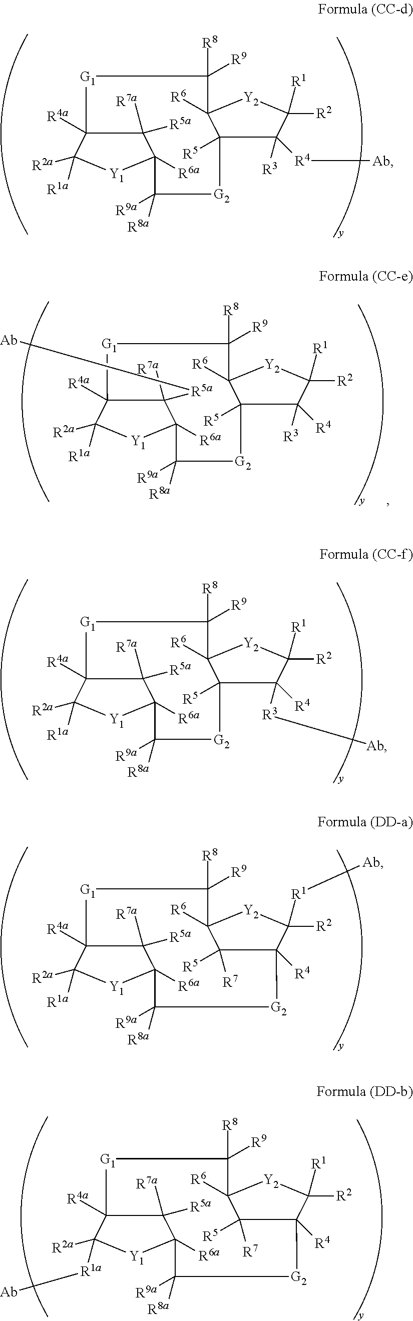

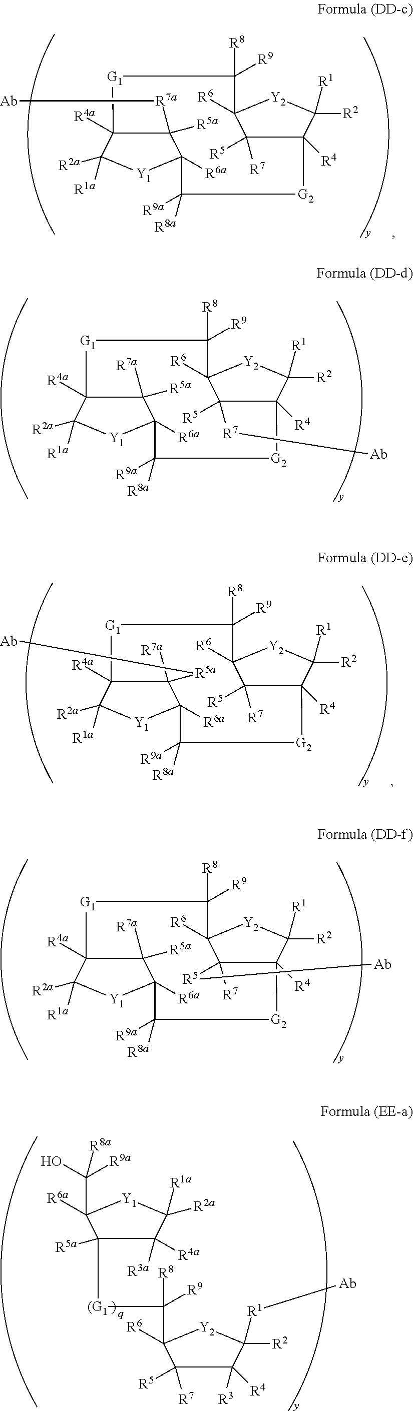

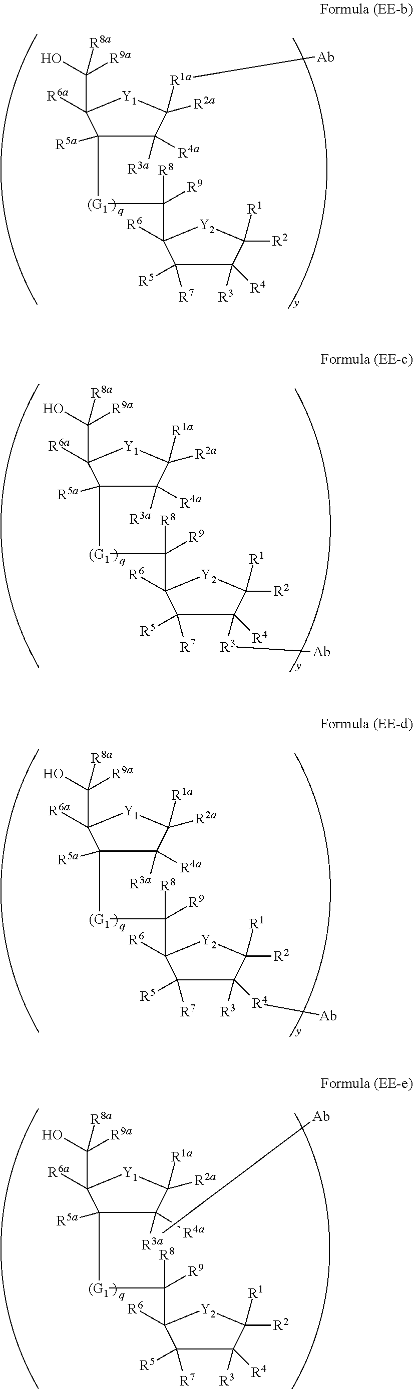

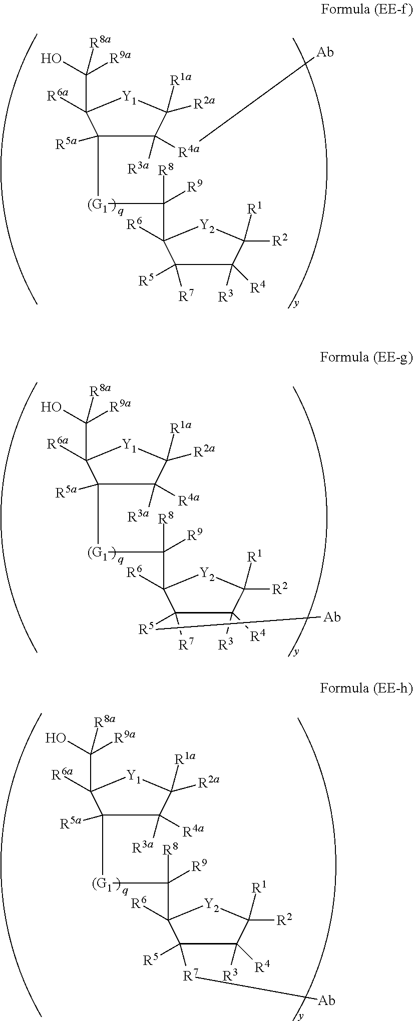

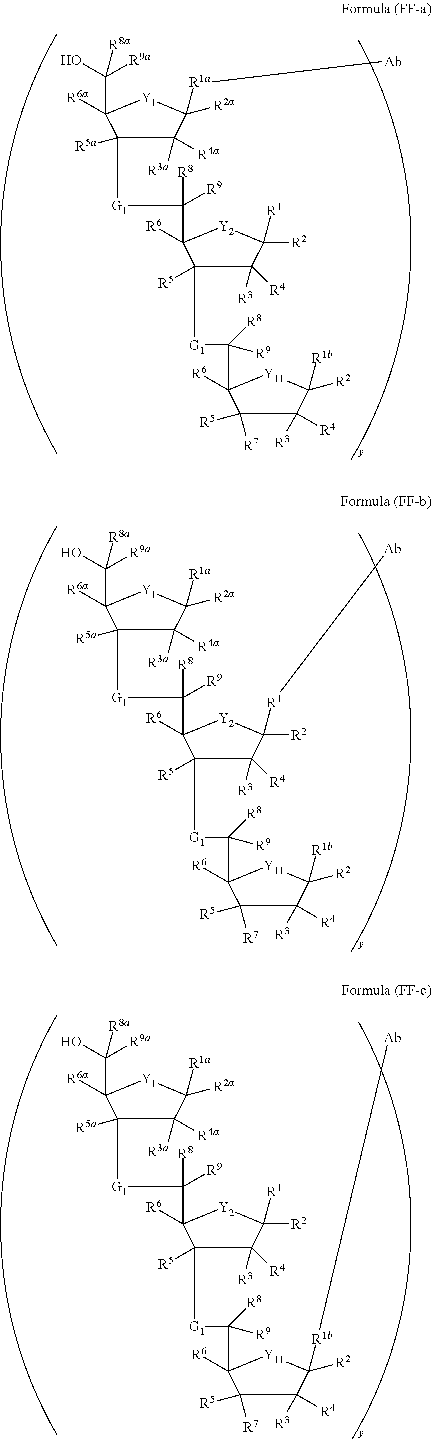

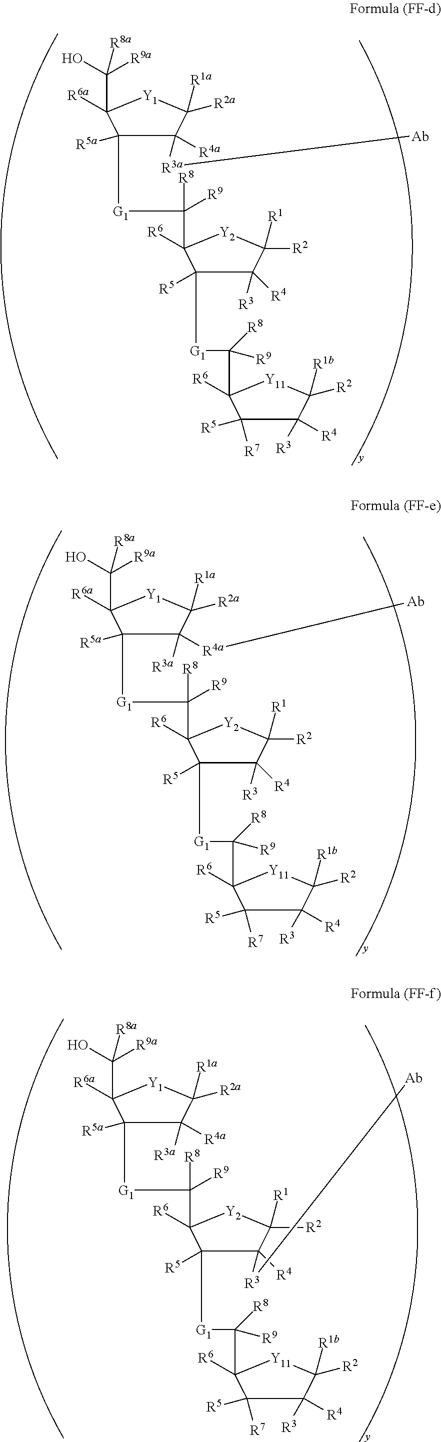

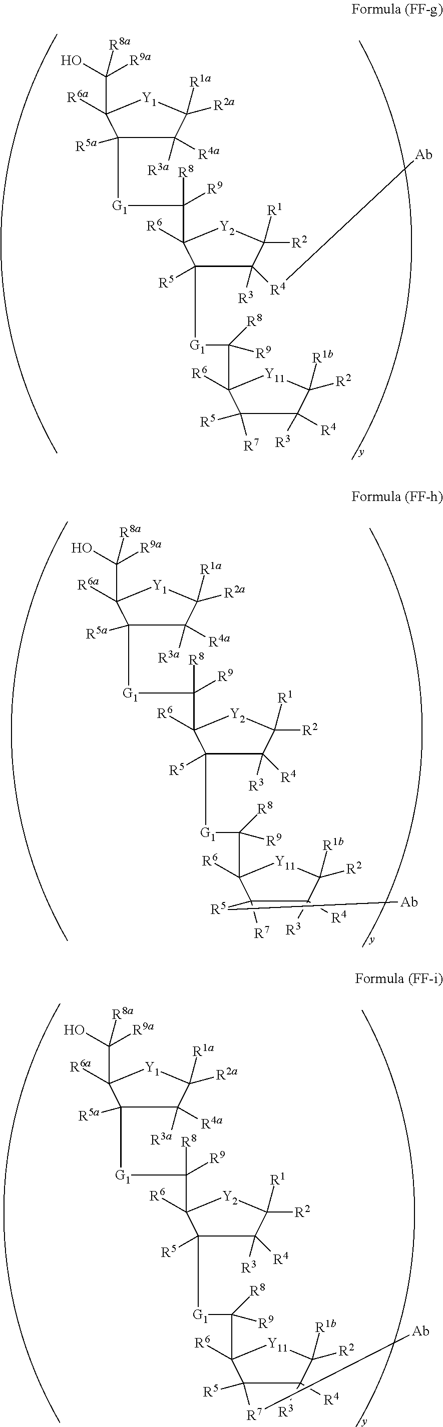

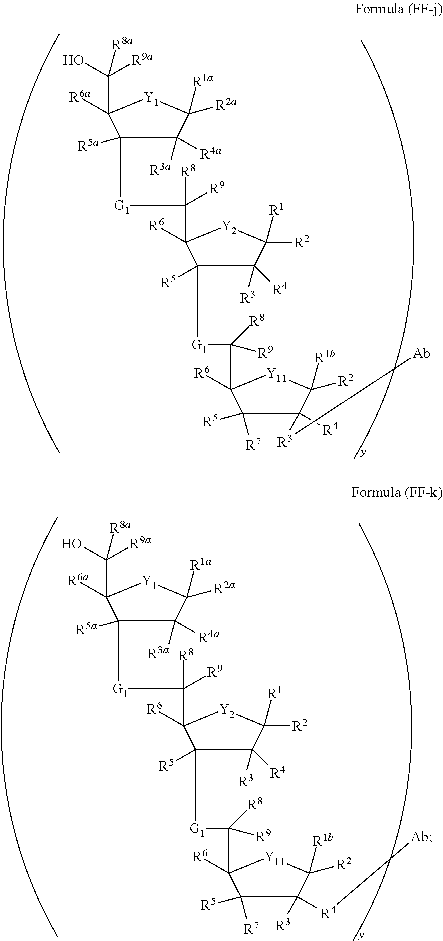

wherein:
each G₁ is independently selected from

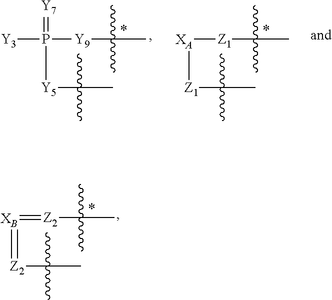

where the * of G₁ indicates the point of attachment to —CR⁸R⁹—;
X_{A} is C(═O)—, —C(═S)— or —C(═NR¹¹)— and each Z₁ is NR¹²;
X_{B} is C, and each Z₂ is N;

### G₂ is

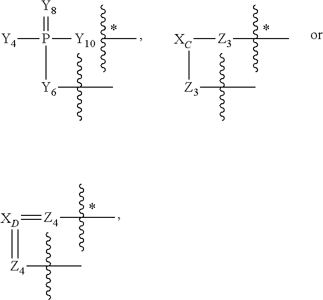

where the * of G₂ indicates the point of attachment to —CR^{8a}R^{9a}—;
X_{C} is C(═O)—, —C(═S)— or —C(═NR¹¹)— and each Z₃ is NR¹²;
X_{D} is C, and each Z₄ is N;
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₃ is OH, O⁻, OR¹⁰, N(R¹⁰)₂, SR¹⁰, SeH, Se⁻, BH₃, SH or S⁻;
Y₄ is OH, O⁻, OR¹⁰, N(R¹⁰)₂, SR¹⁰, SeH, Se⁻, BH₃, SH or S⁻;
Y₅ is —CH₂—, —NH—, —O— or —S;
Y₆ is —CH₂—, —NH—, —O— or —S;

### Y₇ is O or S;

### Y₈ is O or S;

Y₉ is —CH₂—, —NH—, —O— or —S;
Y₁₀ is —CH₂—, —NH—, —O— or —S;
Y₁₁ is —O—, —S—, —S(═O)—, —SO₂—, —CH₂—, or —CF₂—;
q is 1, 2 or 3;
each R¹ is independently partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R¹ is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R^{1a} is independently partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1a} is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R^{1b} is independently partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1b} is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
each R⁸ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁸ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R⁹ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁹ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁹ are substituted by 0, 1, 2 or 3 substituents independently selected from F, Cl, Br, I, OH, CN, and N₃;
each R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
each R^{3a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
each R^{4a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
each R^{5a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
each R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
each R^{7a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
each R^{8a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{8a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R^{9a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{9a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{9a} are substituted by 0, 1, 2 or 3 substituents independently selected from F, Cl, Br, I, OH, CN, and N₃;
each R¹⁰ is independently selected from the group consisting of H, C₁-C₁₂alkyl, C₁-C₆heteroalkyl, —(CH₂CH₂O)ₙCH₂CH₂C(═O)OC₁-C₆alkyl, and

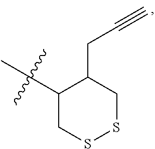 [SMILES: *C1CSSCC1CC#C]

wherein the C₁-C₁₂alkyl and C₁-C₆heteroalkyl of R¹⁰ is substituted by 0, 1, 2 or 3 substituents independently selected from —OH, C₁-C₂alkoxy, —S—C(═O)C₁-C₆alkyl, halo, —CN, C₁-C₁₂alkyl, —O-aryl, _O-heteroaryl, —O-cycloalkyl, oxo, cycloalkyl, heterocyclyl, aryl, or heteroaryl, —OC(O)OC₁-C₆alkyland C(O)OC₁-C₆alkyl, wherein each alkyl, cycloalkyl, heterocyclyl, aryl, and heteroaryl is substituted by 0, 1, 2 or 3 substituents independently selected from C₁-C₁₂ alkyl, O—C₁-C₁₂alkyl, C₁-C₁₂heteroalkyl, halo, CN, OH, oxo, aryl, heteroaryl, O-aryl, O-heteroaryl, —C(═O)C₁-C₁₂alkyl, —OC(═O)C₁-C₁₂alkyl, —C(═O)OC₁-C₁₂alkyl, —OC(═O)OC₁-C₁₂alkyl, —C(═O)N(R¹¹)—C₁-C₁₂alkyl, —N(R¹¹)C(═O)—C₁-C₁₂alkyl; —OC(═O)N(R¹¹)—C₁-C₁₂alkyl, —C(═O)-aryl, —C(═O)-heteroaryl, —OC(═O)-aryl, —C(═O)O-aryl, —OC(═O)-heteroaryl, —C(═O)O-heteroaryl, —C(═O)O-aryl, —C(═O)O-heteroaryl, —C(═O)N(R¹¹)-aryl, —C(═O)N(R¹¹)-heteroaryl, —N(R¹¹)C(O)-aryl, —N(R¹¹)₂C(O)-aryl, —N(R¹¹)C(O)-heteroaryl, and S(O)₂N(R¹¹)-aryl;
each R¹¹ is independently selected from H and C₁-C₆alkyl;
each R¹² is independently selected from H and C₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position;
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position;
optionally R⁸ and R⁹ are connected to form a C₁-C₆alkylene, C₂-C₆alkenylene, C₂-C₆alkynylene, and
optionally R^{8a} and R^{9a} are connected to form a C₁-C₆alkylene, C₂-C₆alkenylene, O₂—C₆alkynylene,
L₁ is a linker;
Each R¹¹⁵ is independently

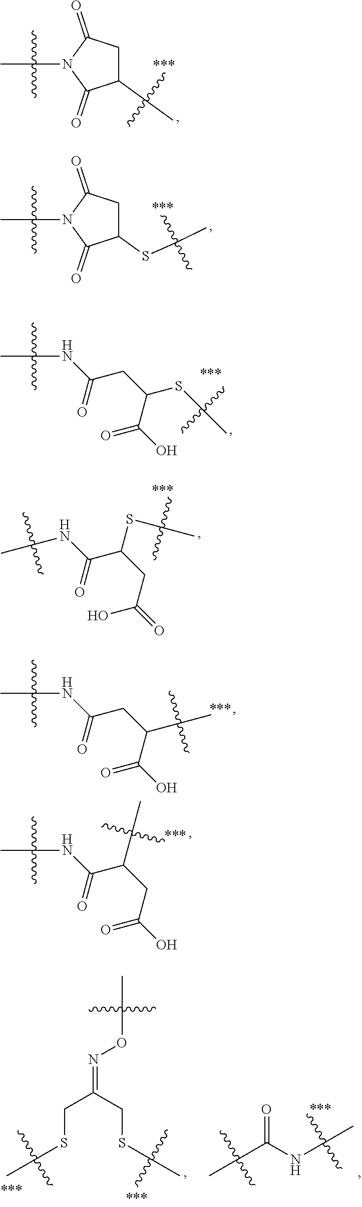

C(═O), —ON═***, —S—, —NHC(═O)CH₂—***, —S(═O)₂CH₂CH₂—***, —(CH₂)₂S(═O)₂CH₂CH₂—***, —NHS(═O)₂CH₂CH₂₋***, —NHC(═O)CH₂CH₂—***, —CH₂NHCH₂CH₂—***, —NHCH₂CH₂—***,

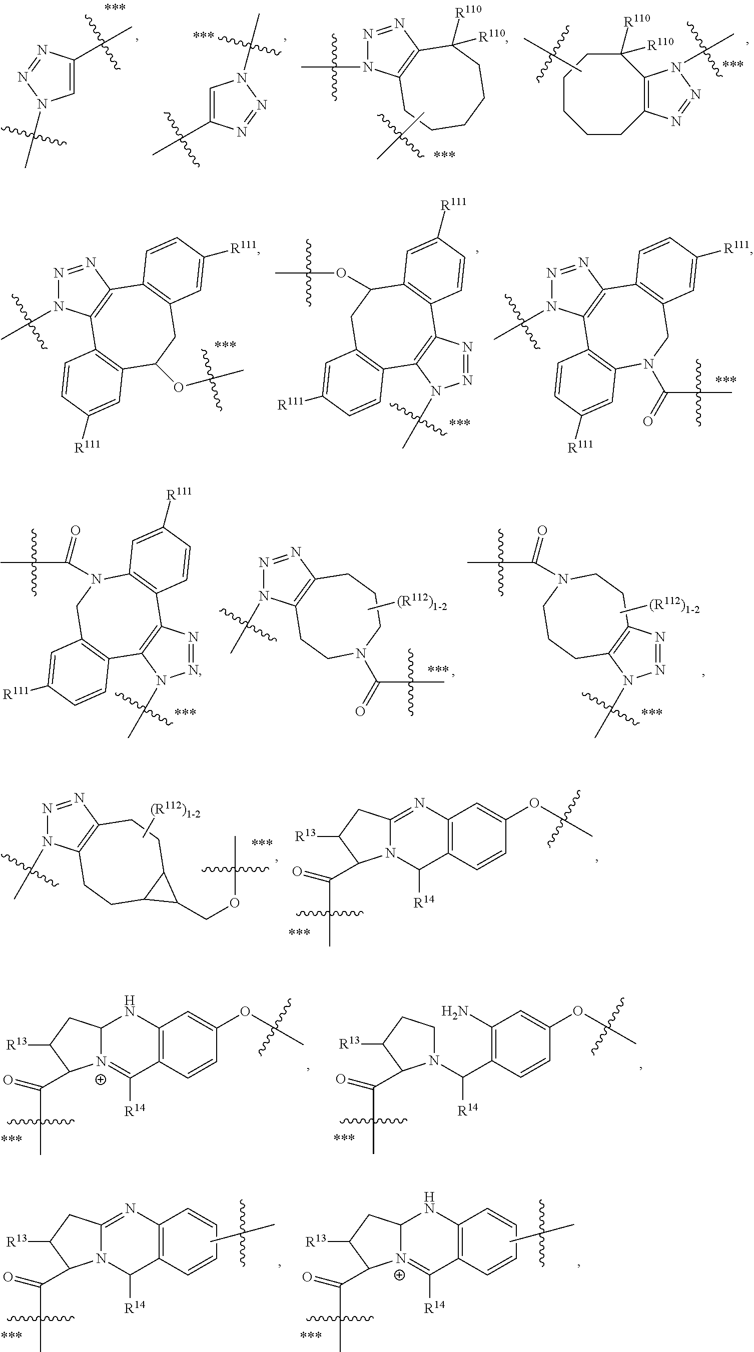

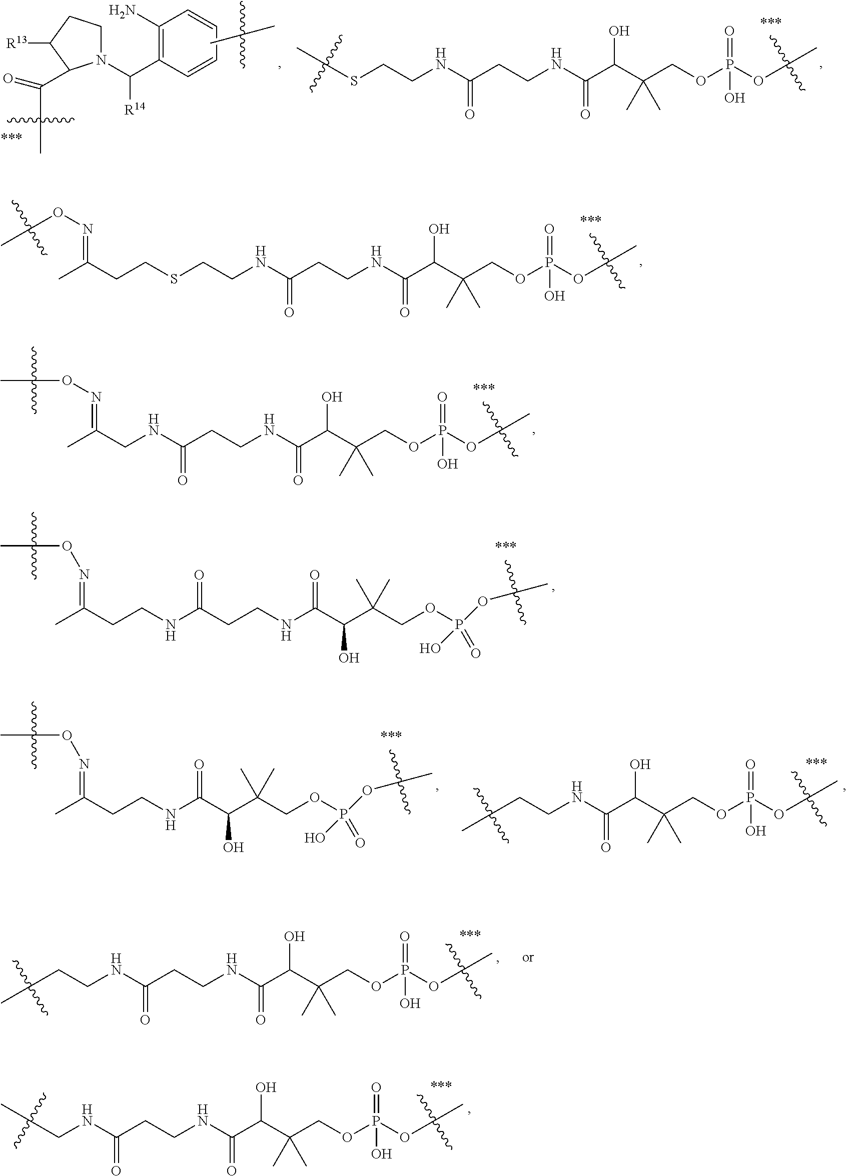

where *** of R¹¹⁵ indicates the point of attachment to Ab;
R¹³ is H or methyl;
R¹⁴ is H, —CH₃ or phenyl;
each R¹¹⁰ is independently selected from H, C₁-C₆alkyl, F, Cl, and —OH;
each R¹¹¹ is independently selected from H, C₁-C₆alkyl, F, Cl, —NH₂, —OCH₃, —OCH₂CH₃, —N(CH₃)₂, —CN, —NO₂ and —OH;
each R¹¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
Ab is an antibody or a functional fragment thereof; and
y is 1, 2, 3, 4, 5, 6, 7, 8, 9 or 10.

In some embodiments disclosed herein, the immunconjugates comprise a structure selected from:

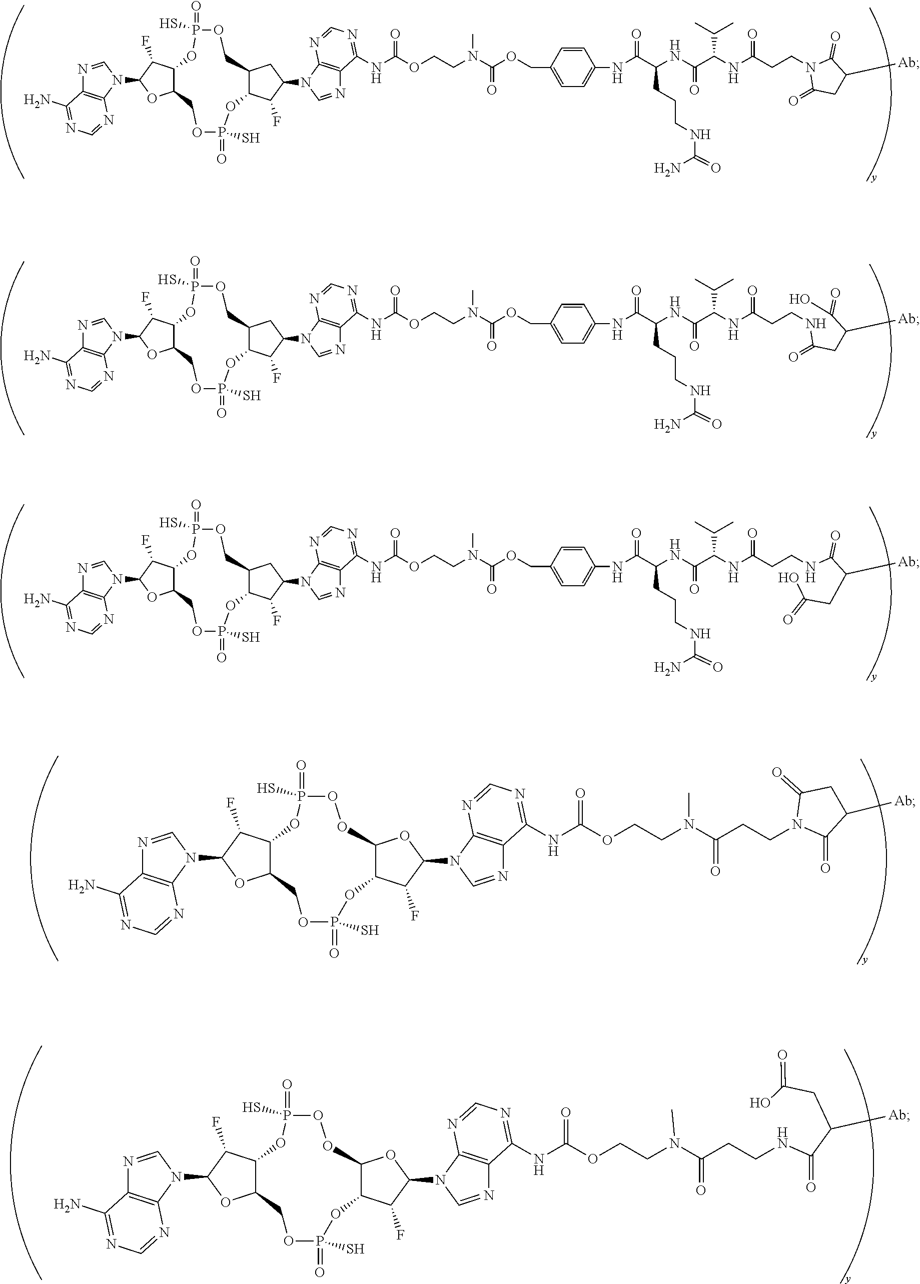

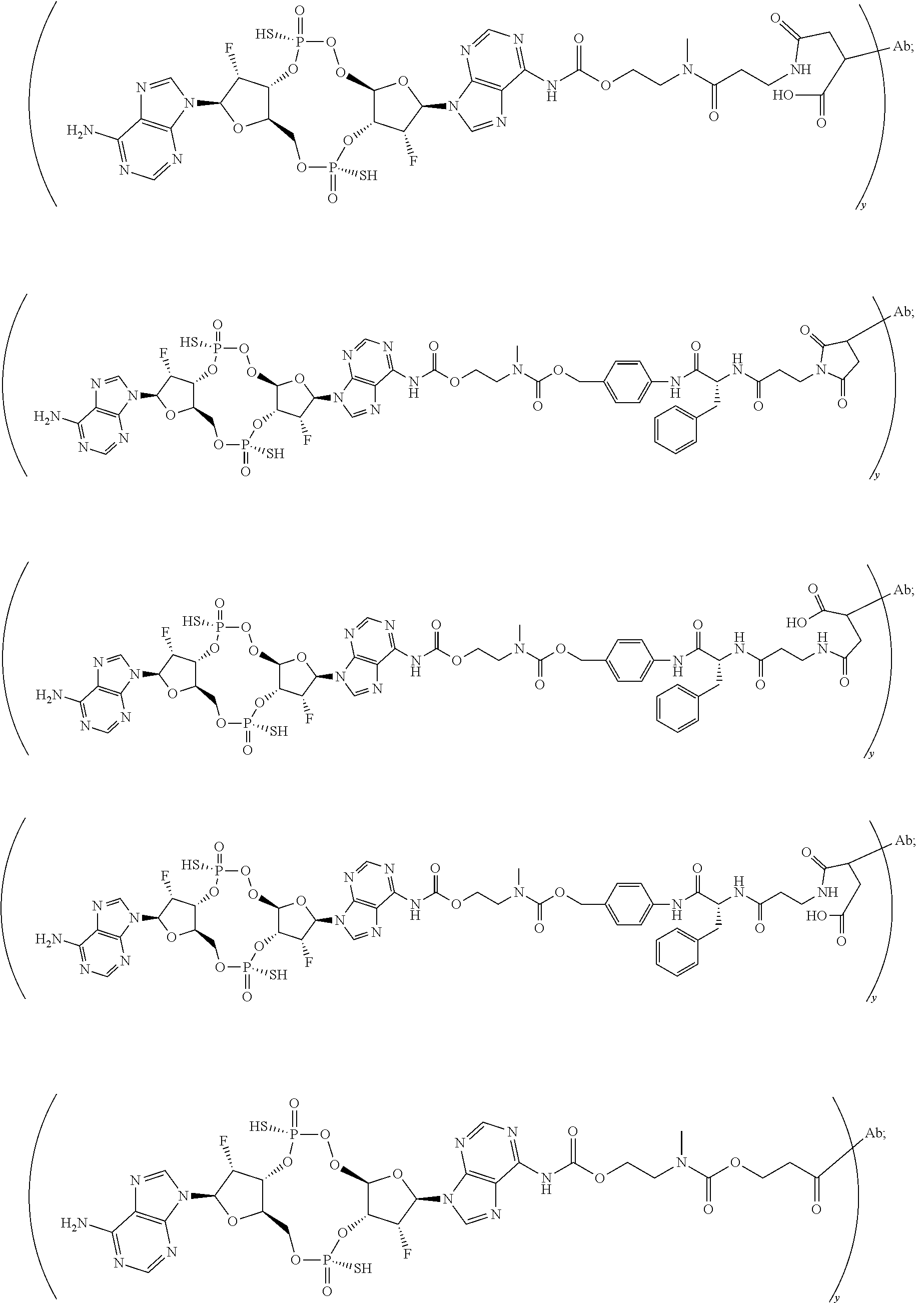

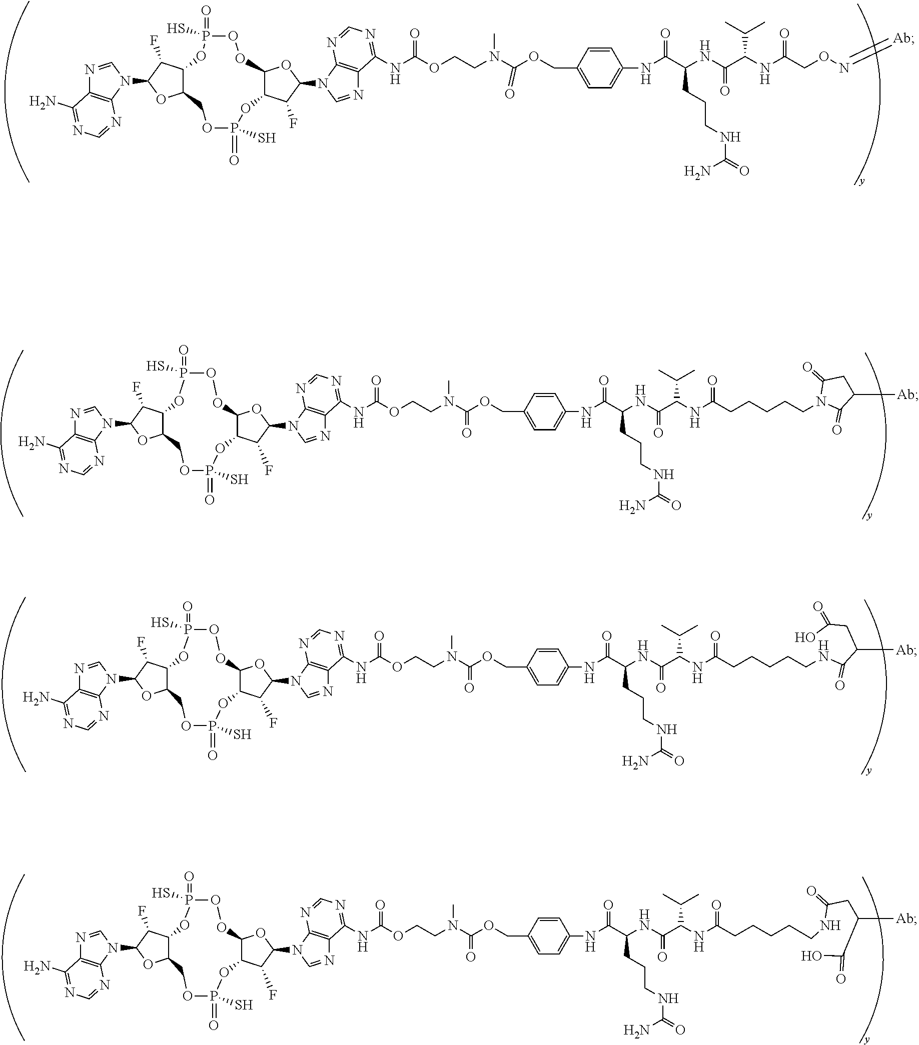

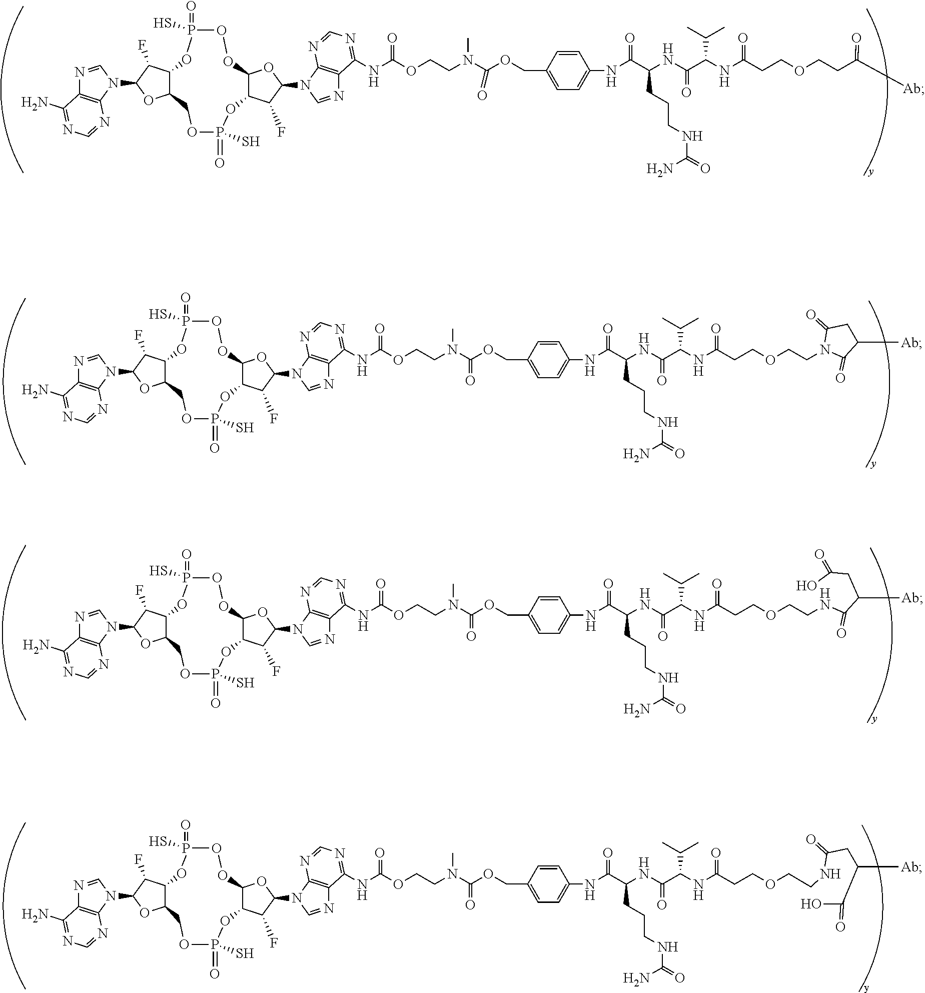

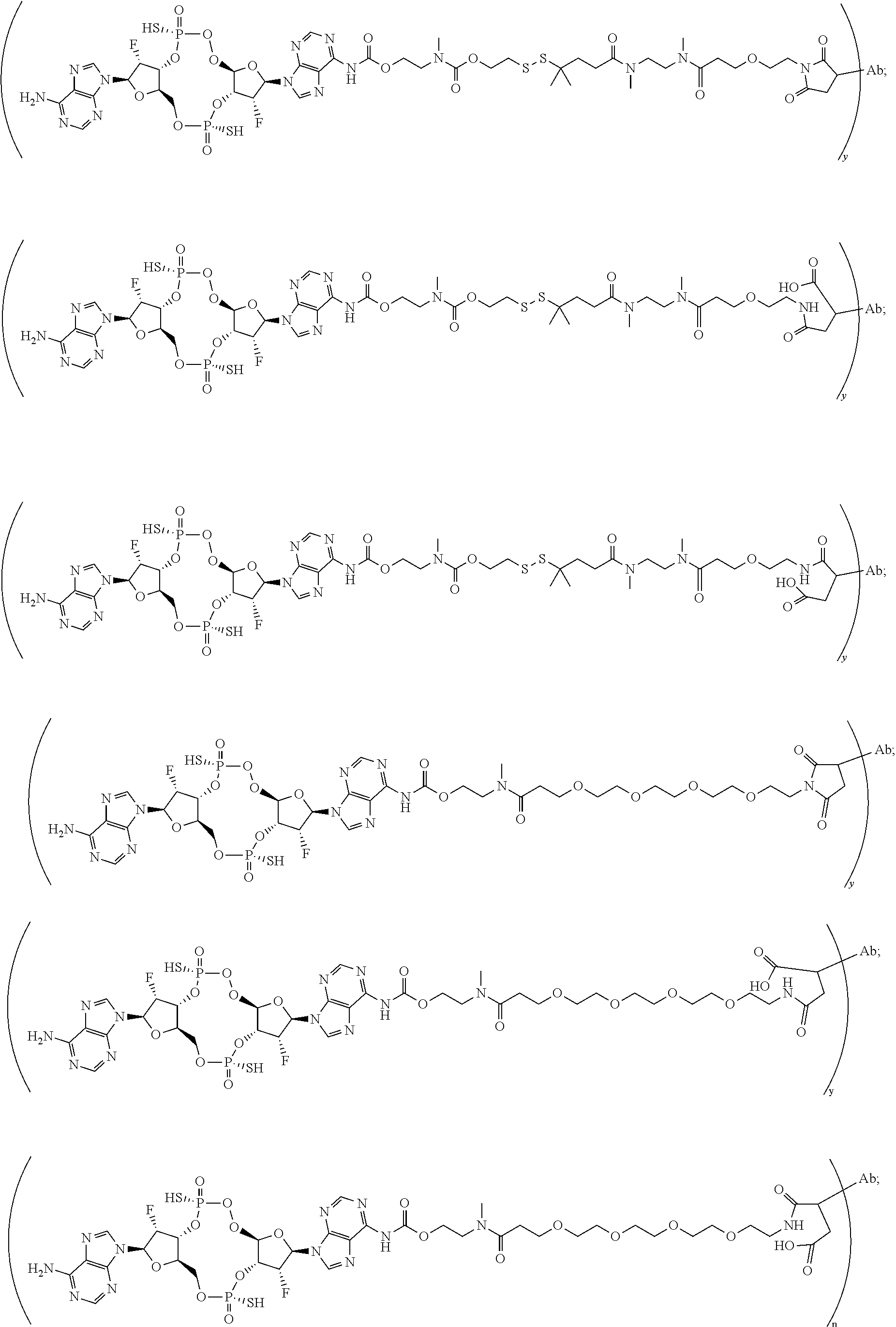

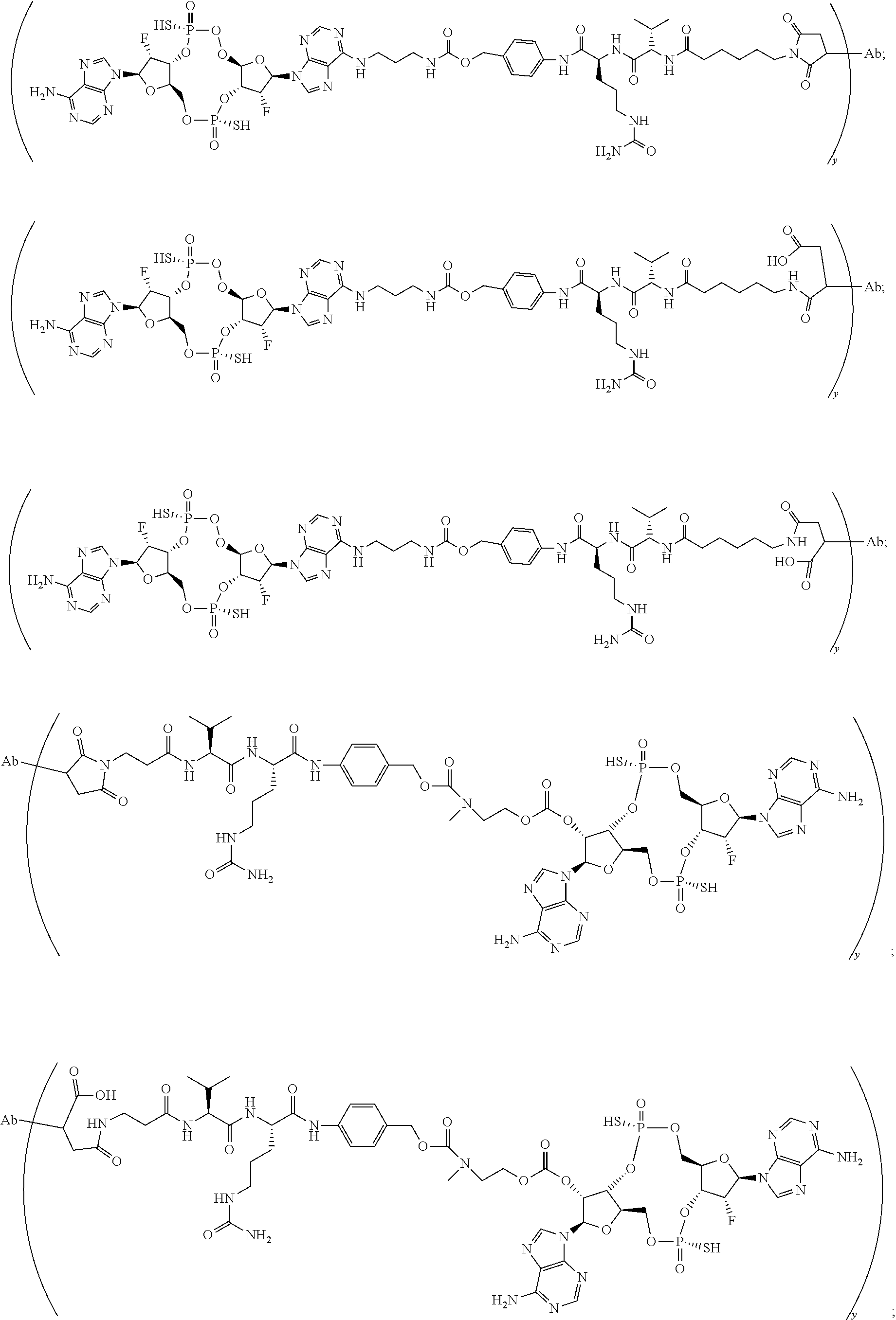

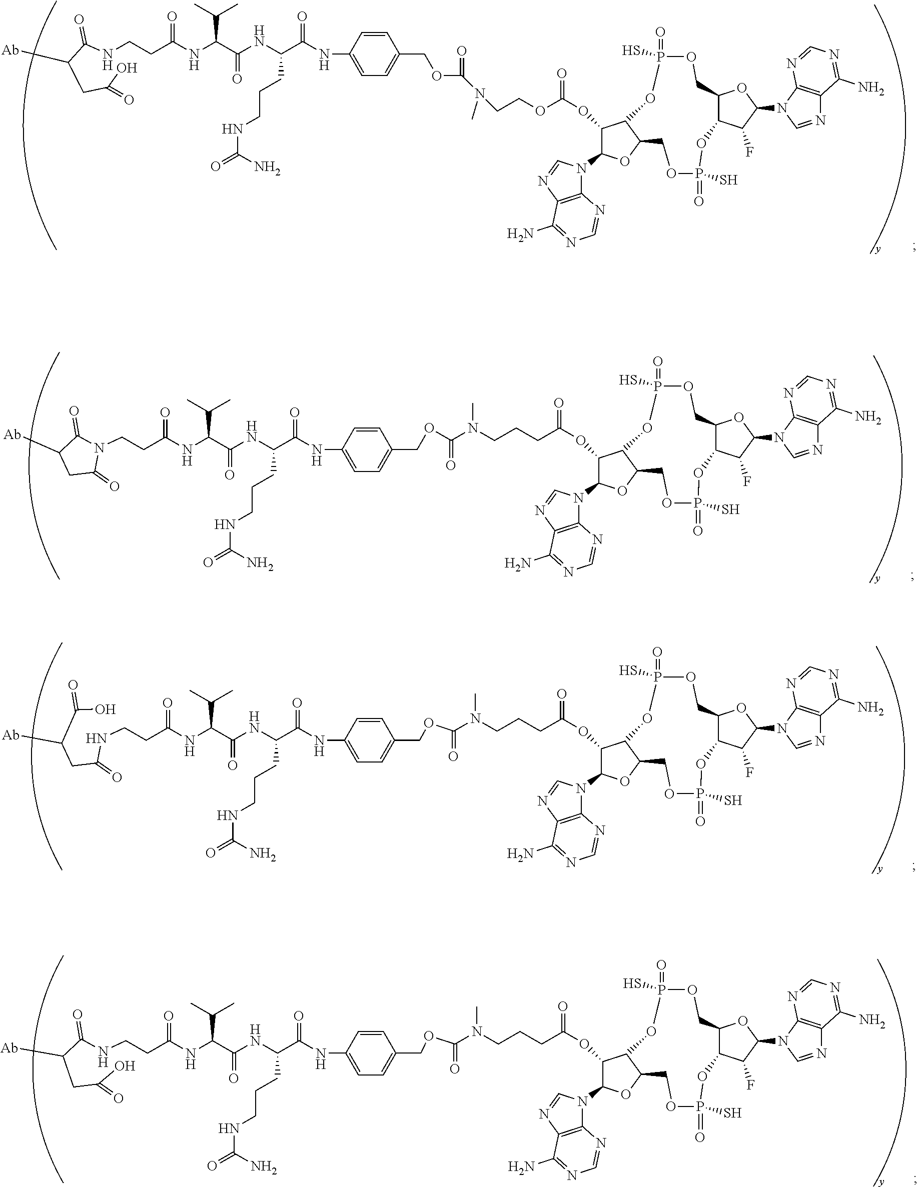

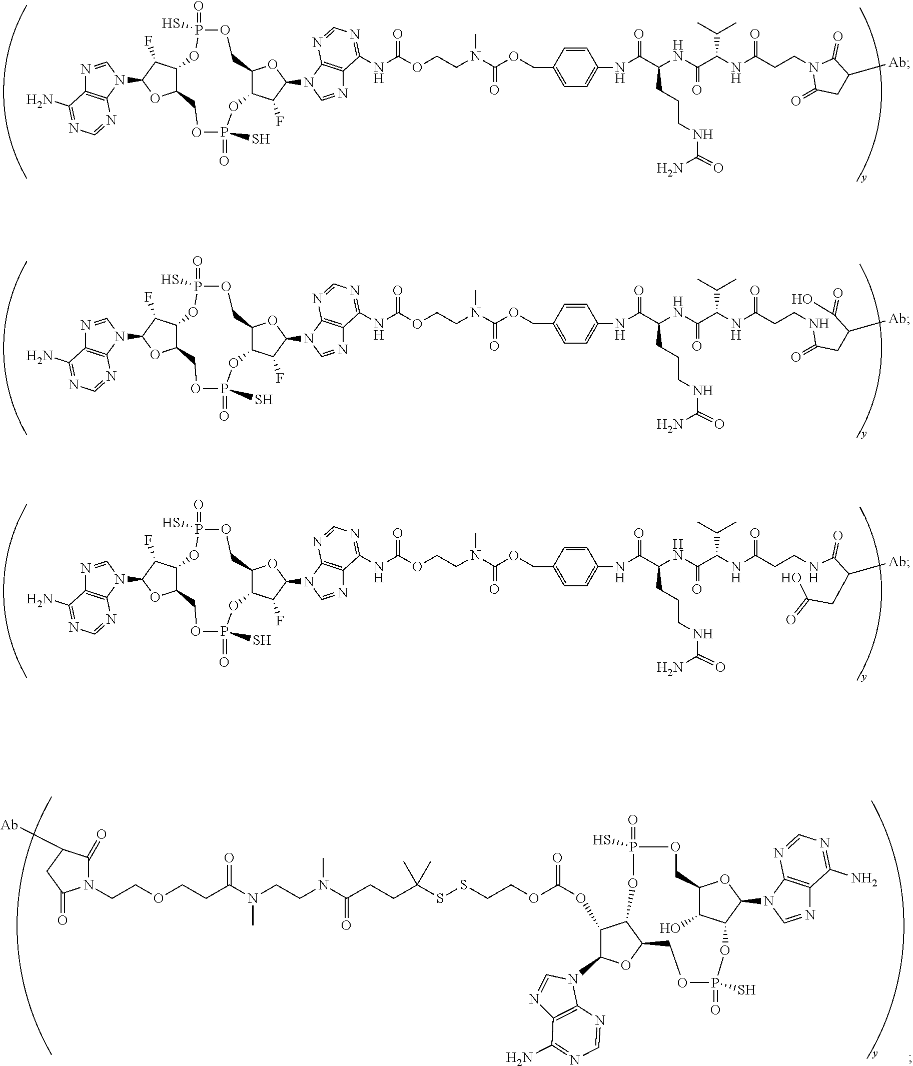

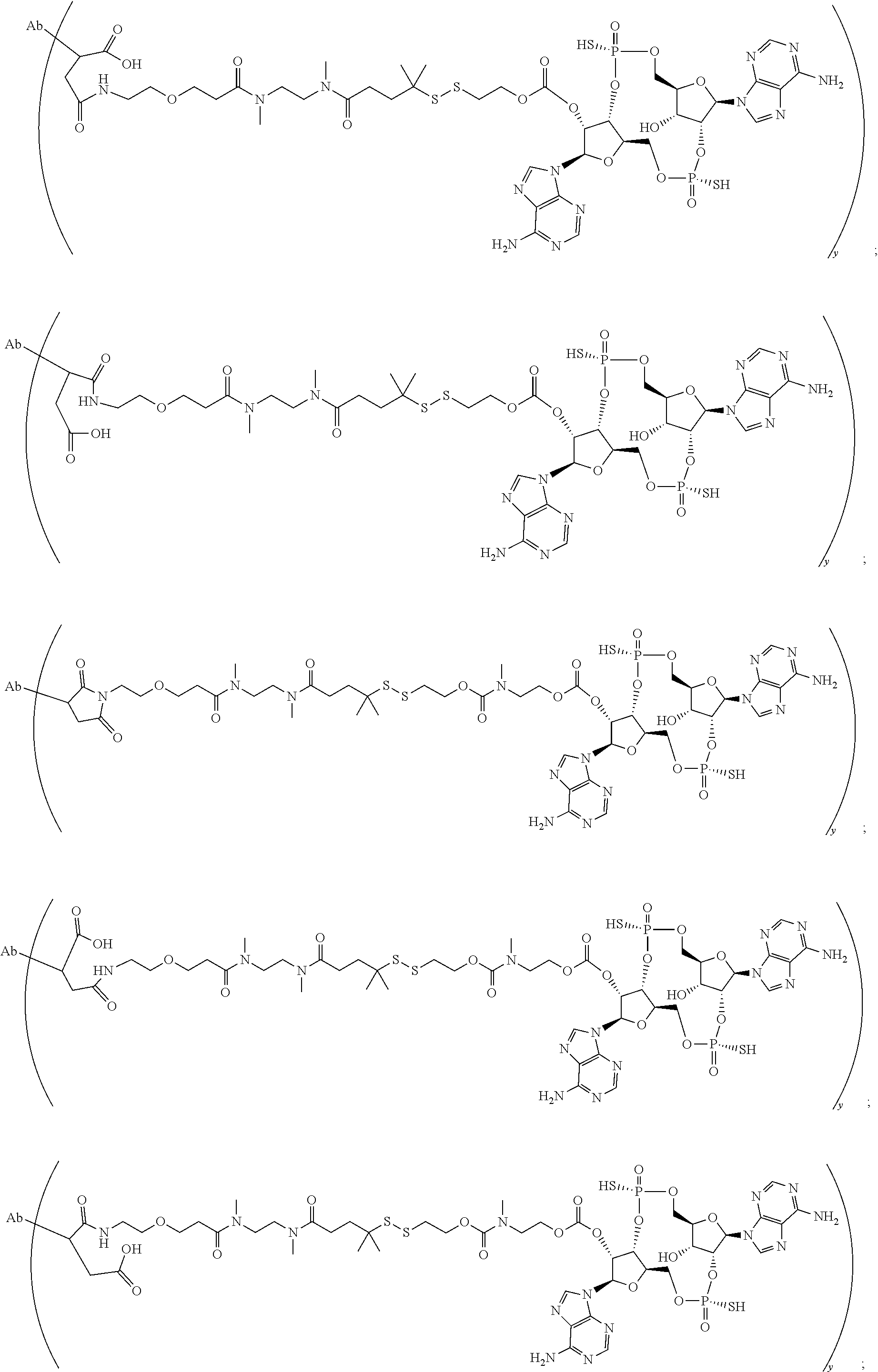

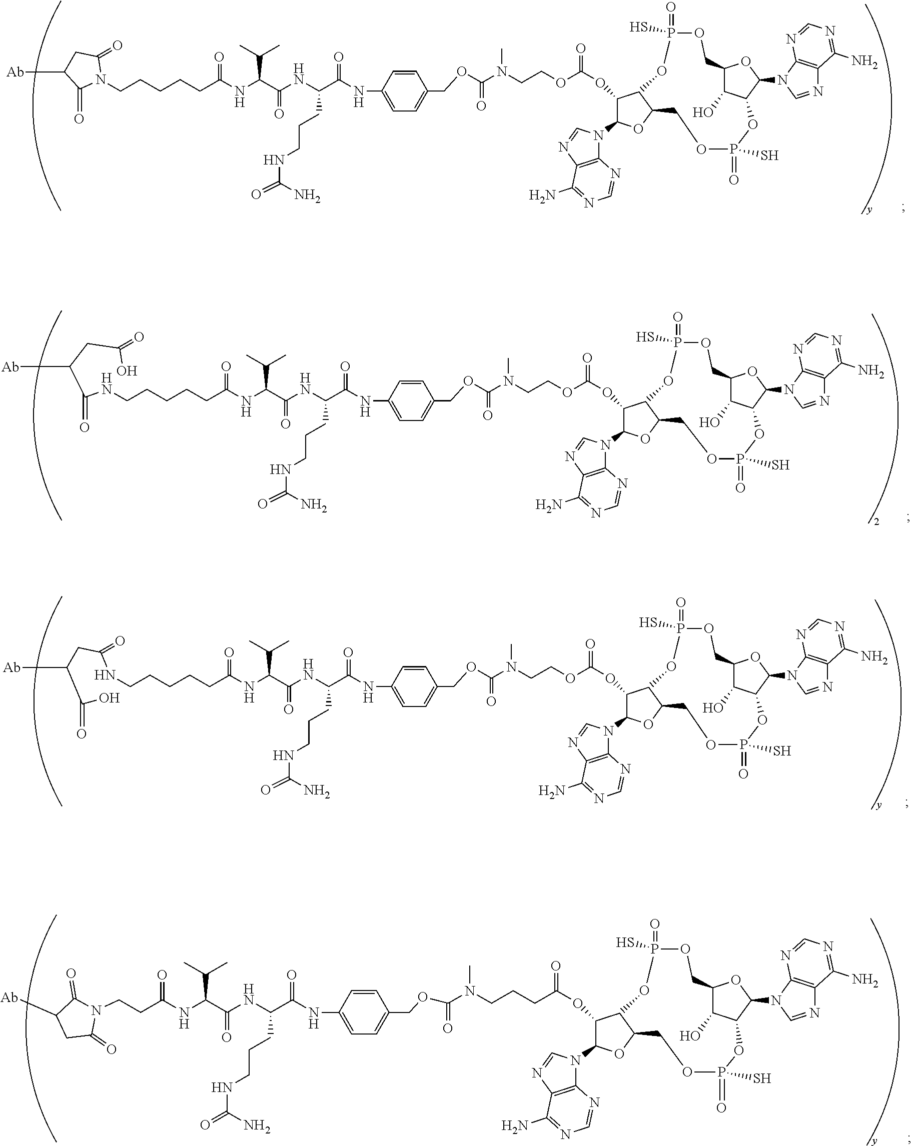

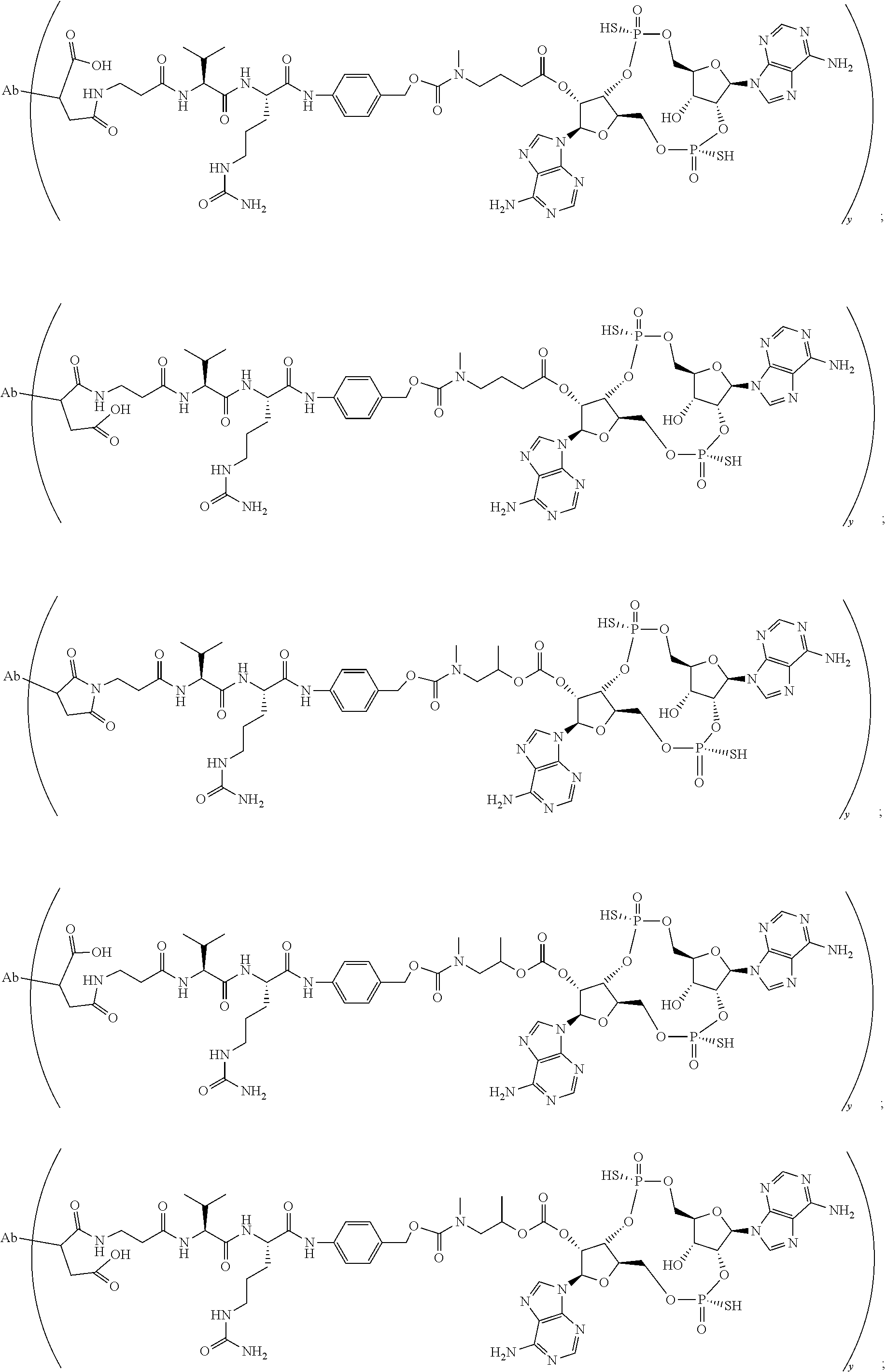

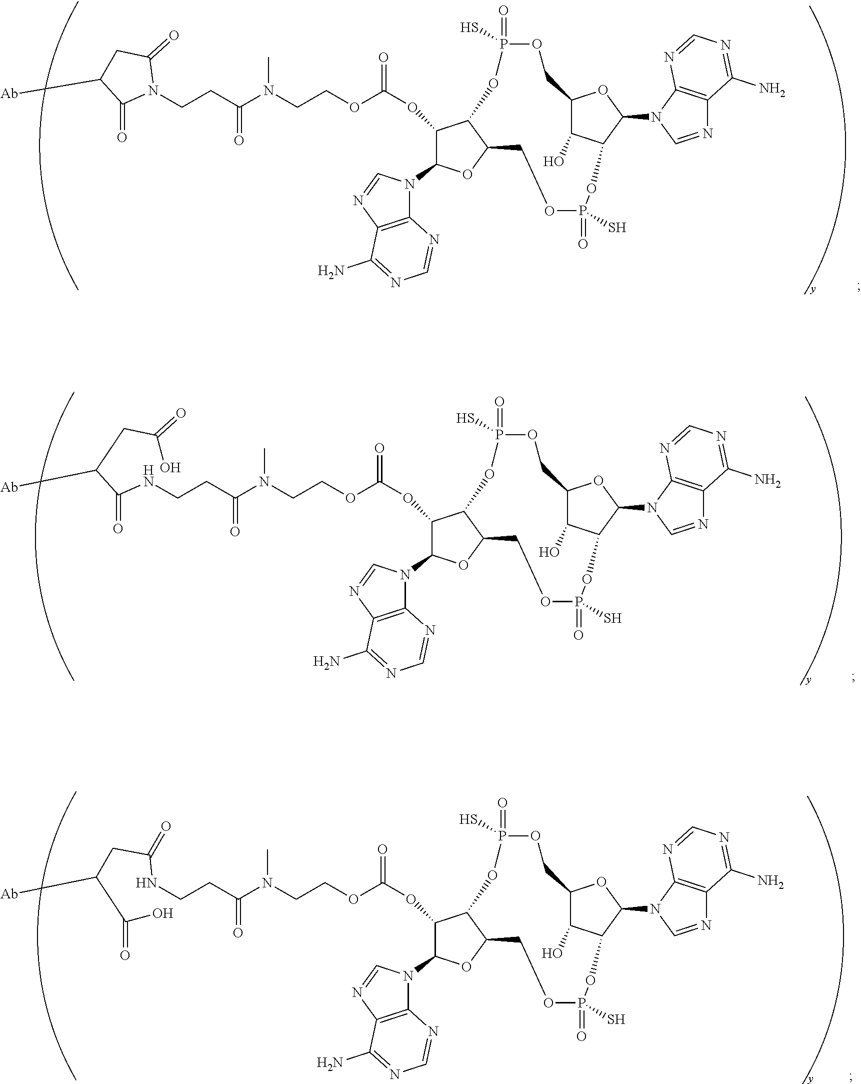

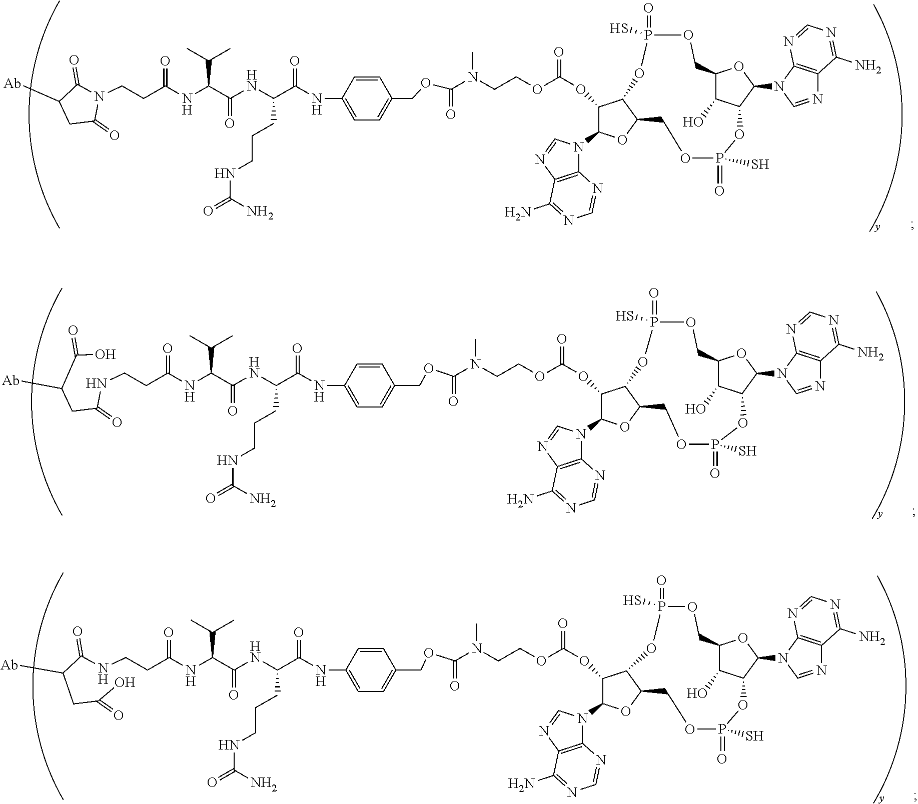

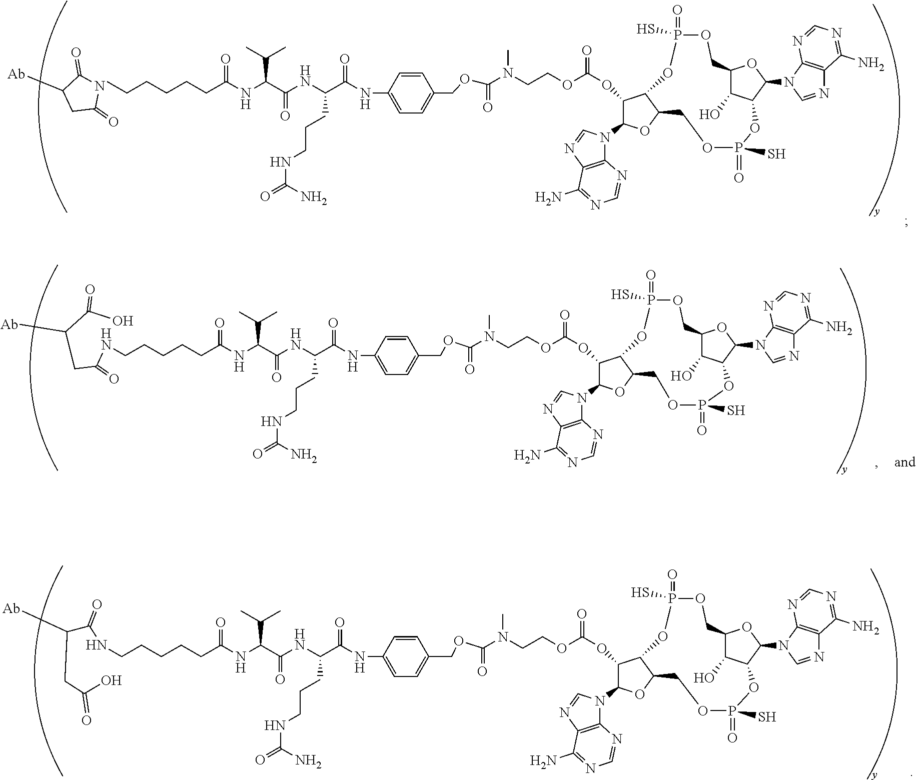

In other embodiments disclosed herein, the immunconjugates comprise a structure selected from:

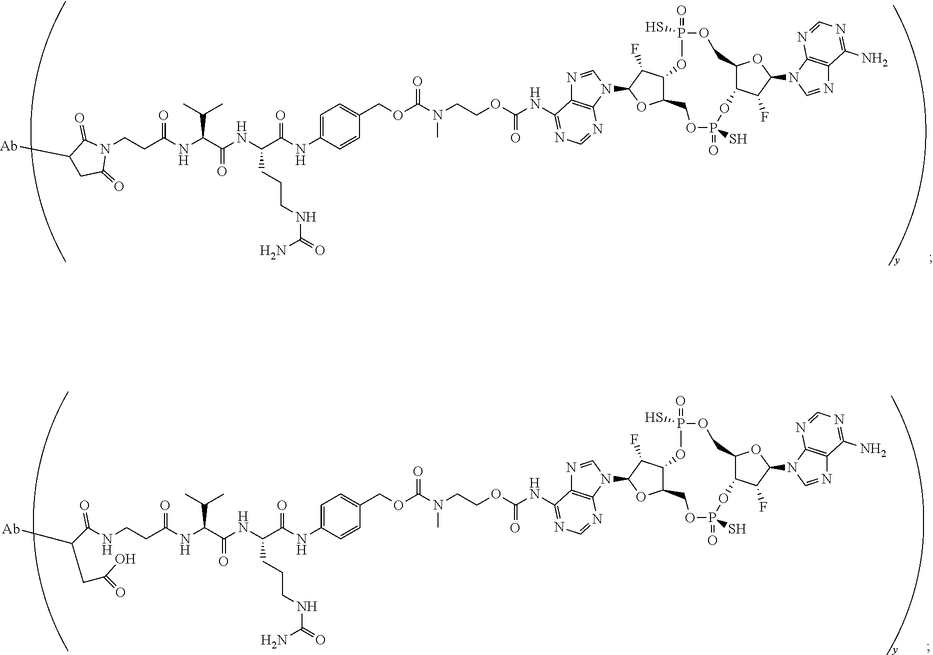

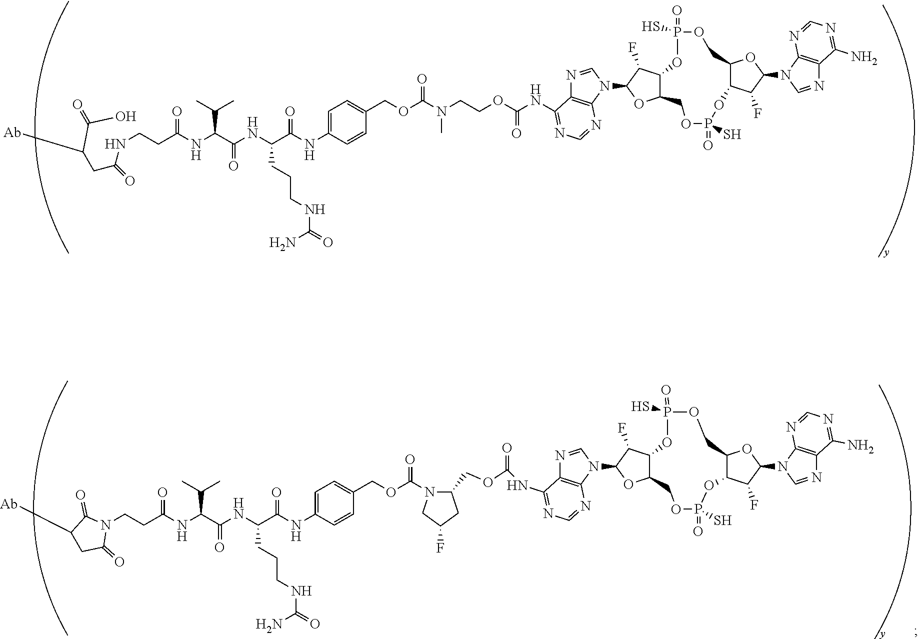

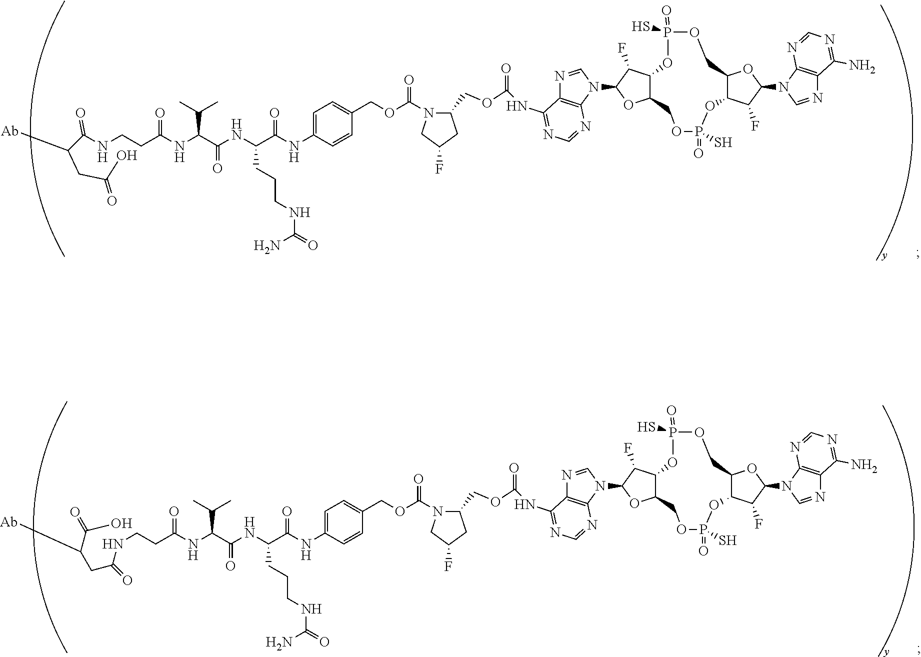

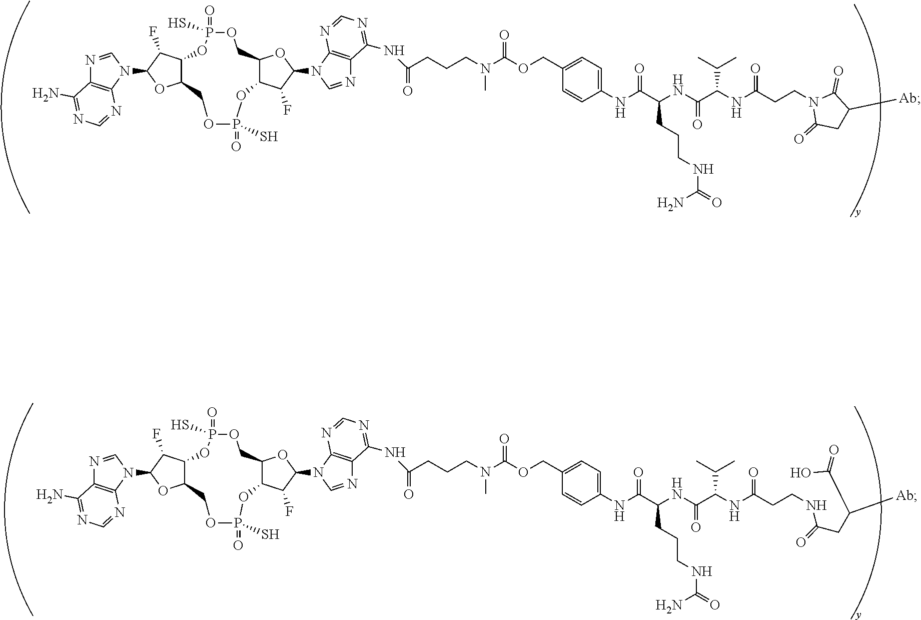

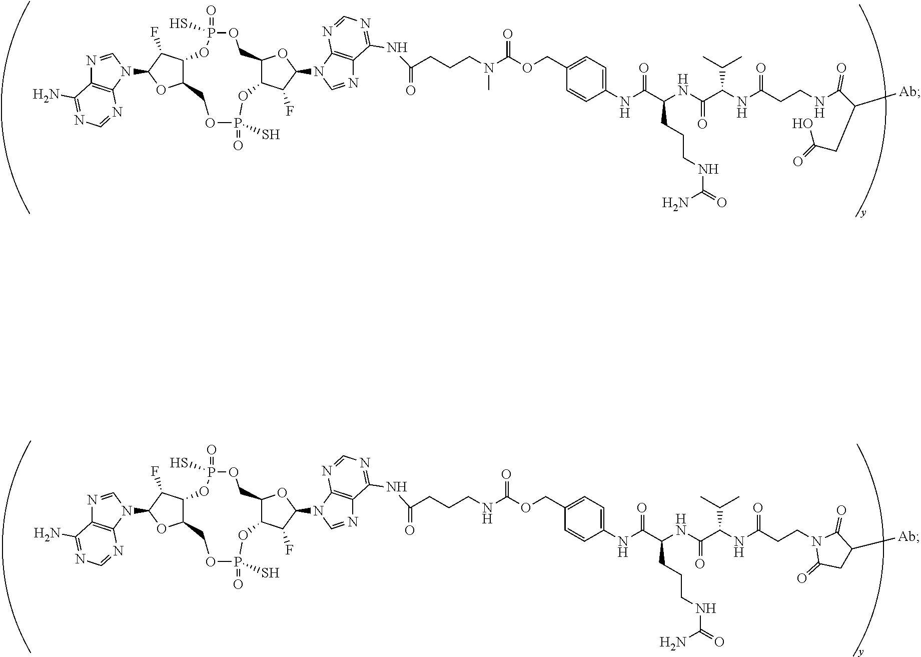

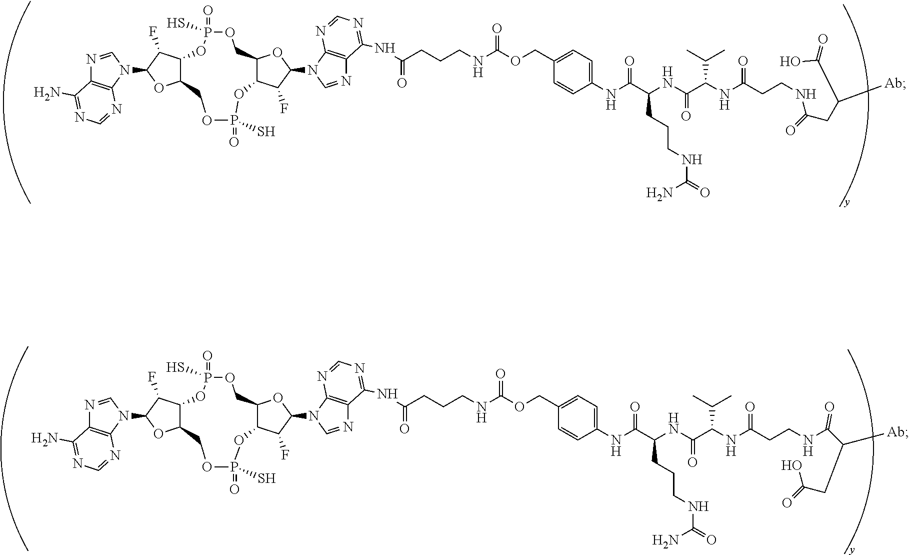

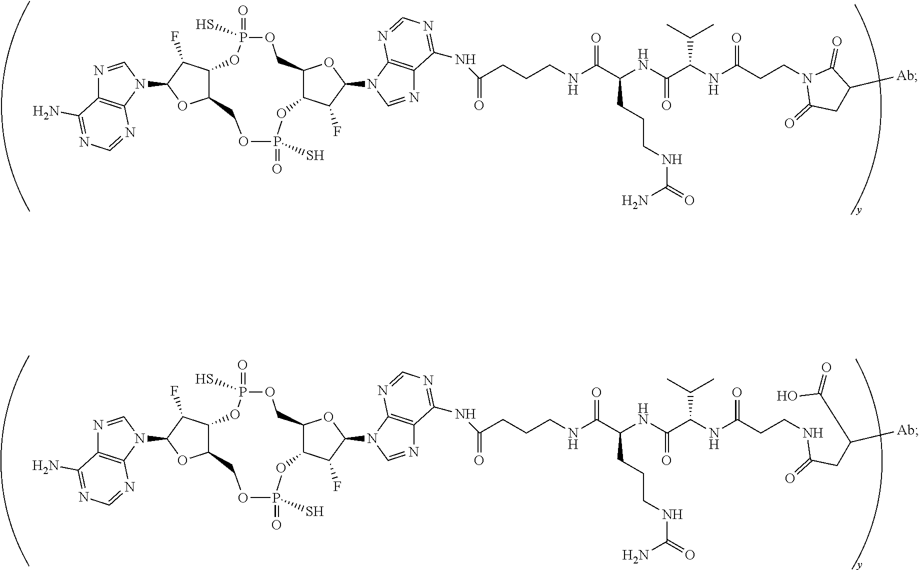

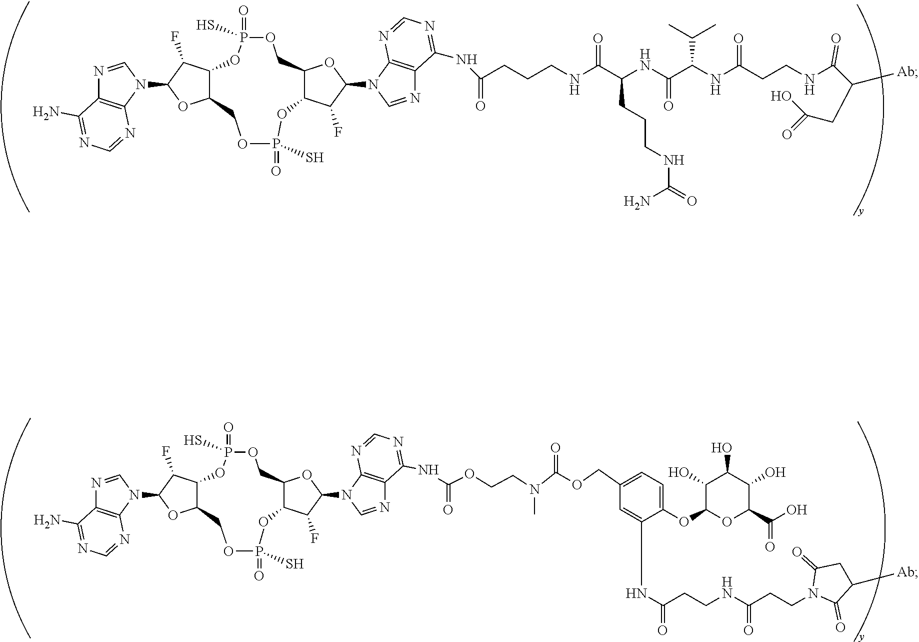

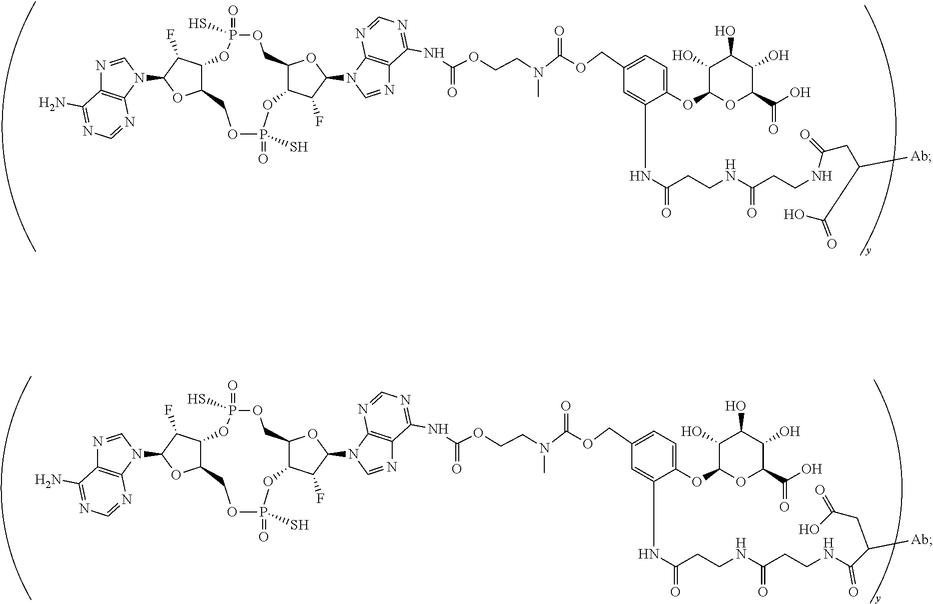

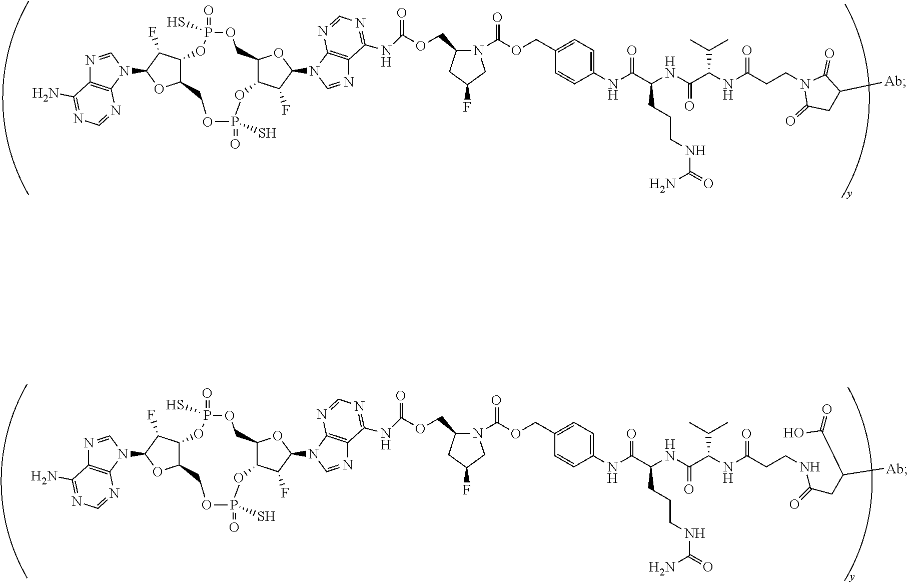

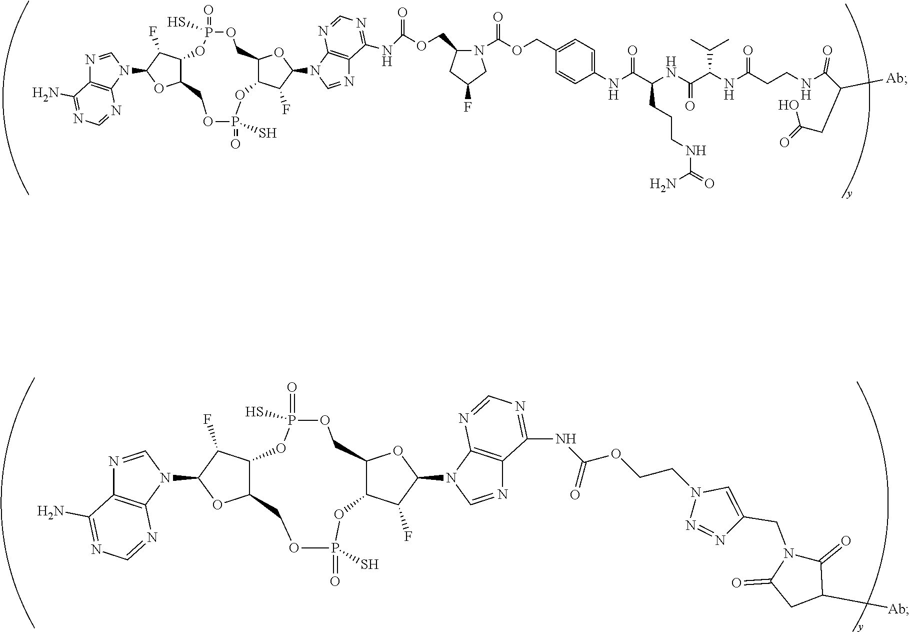

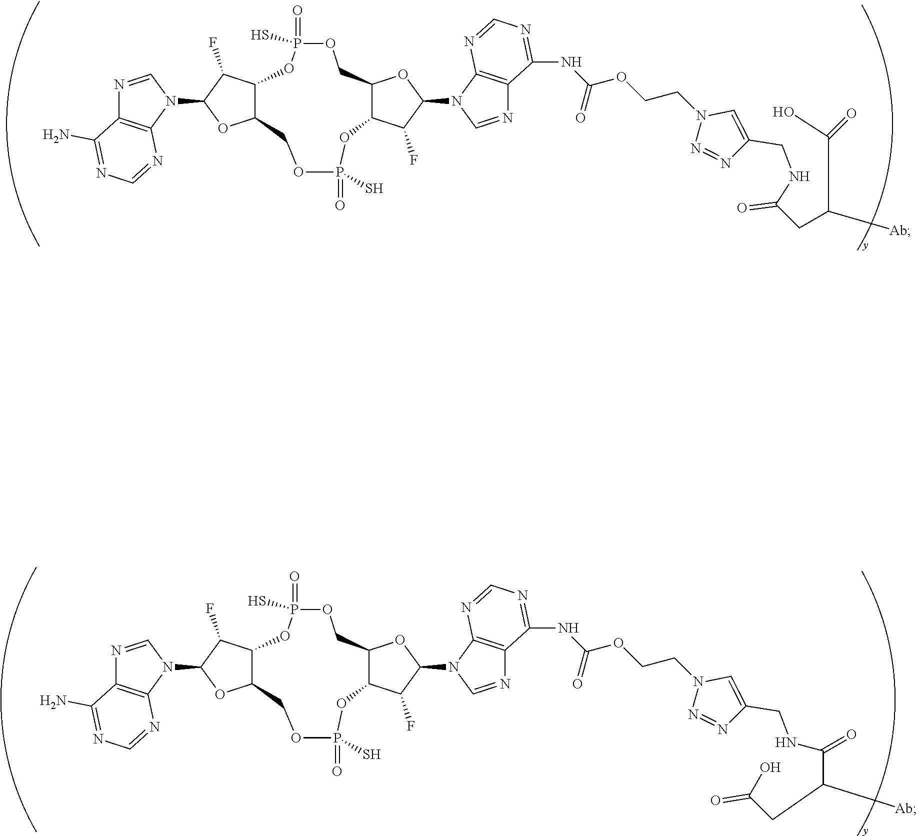

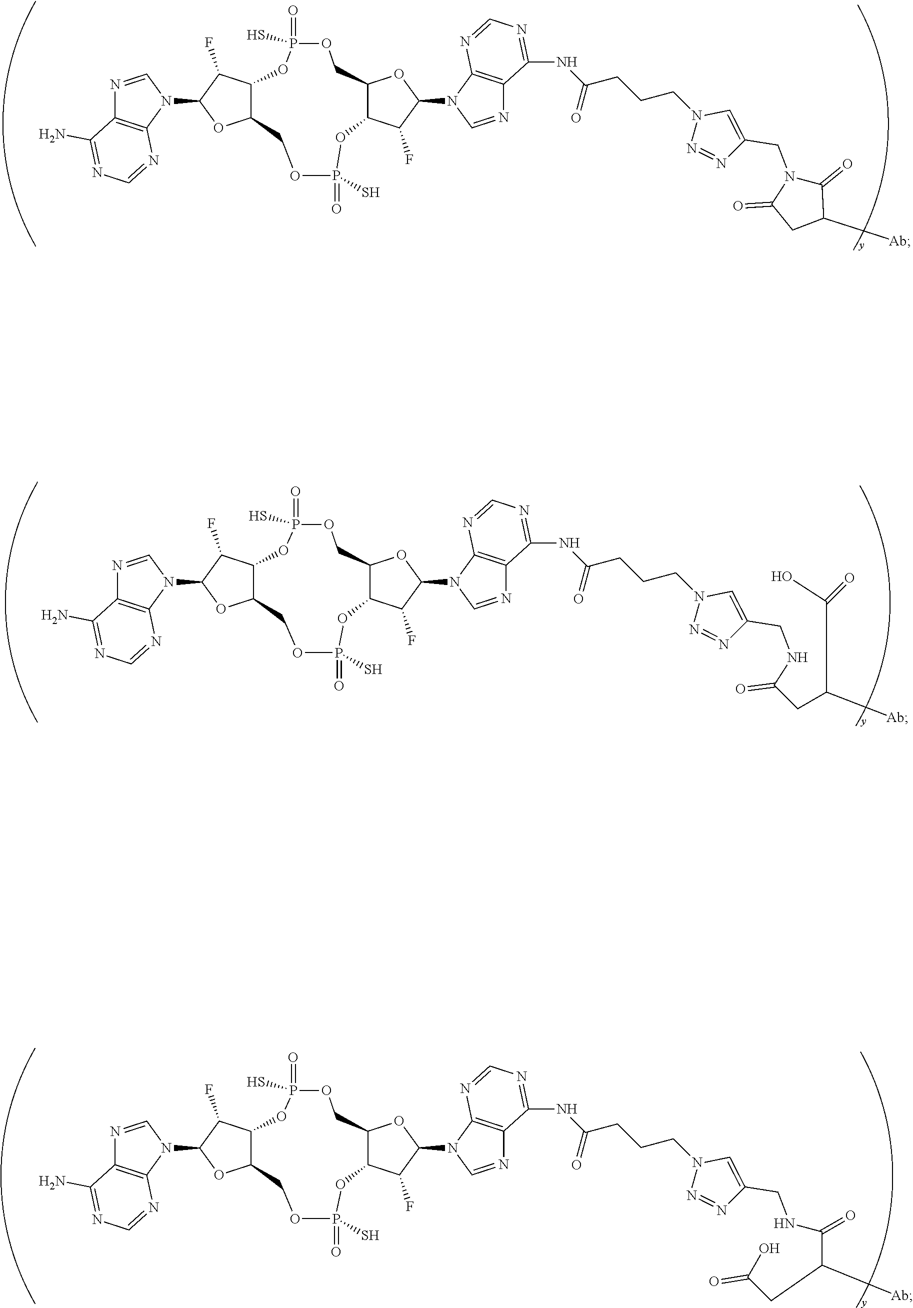

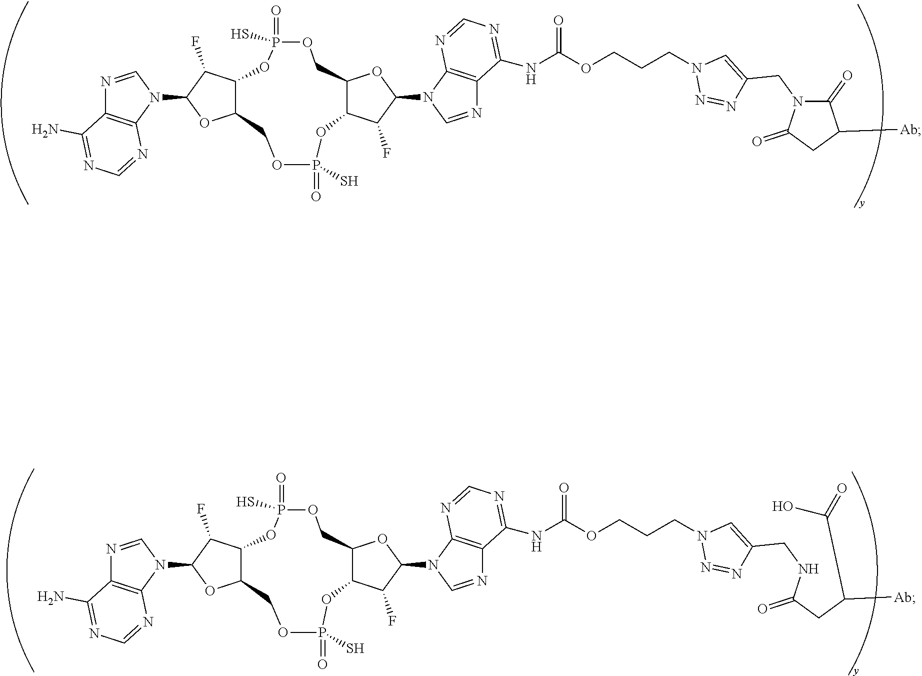

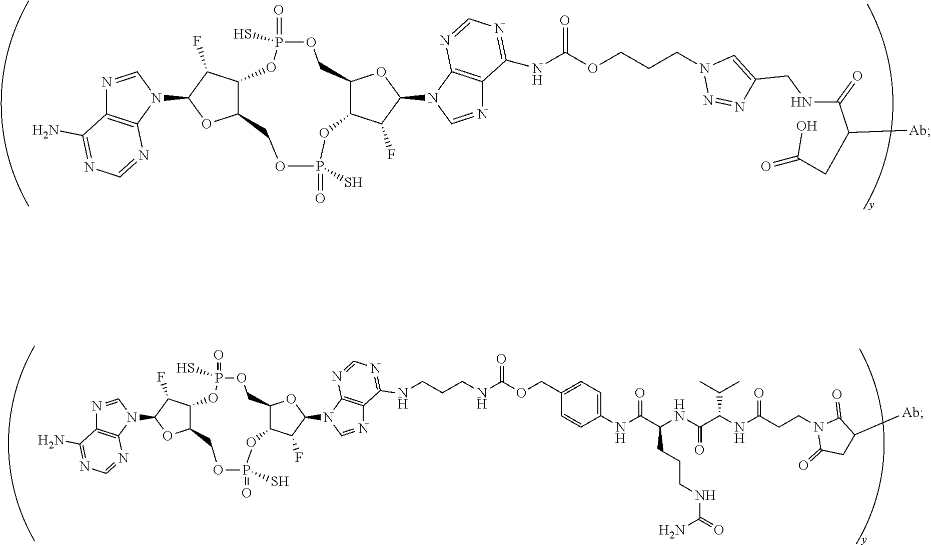

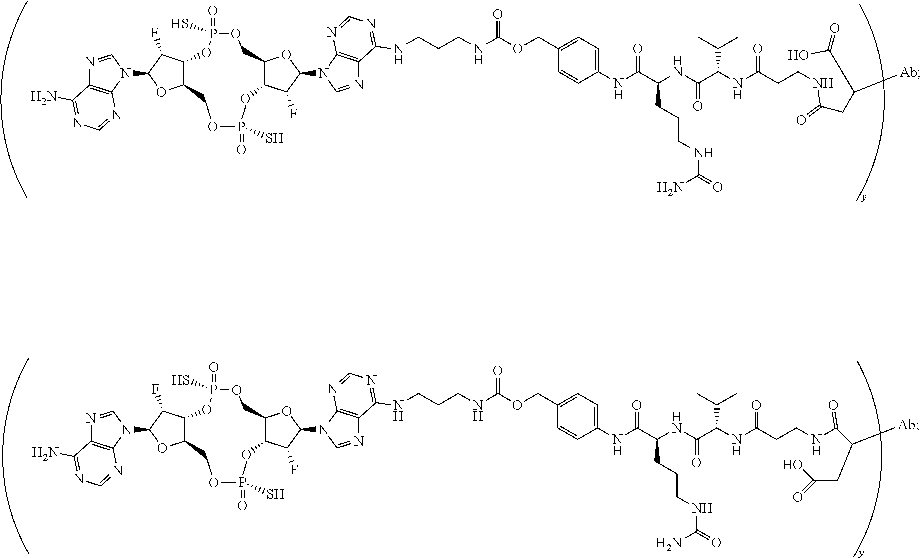

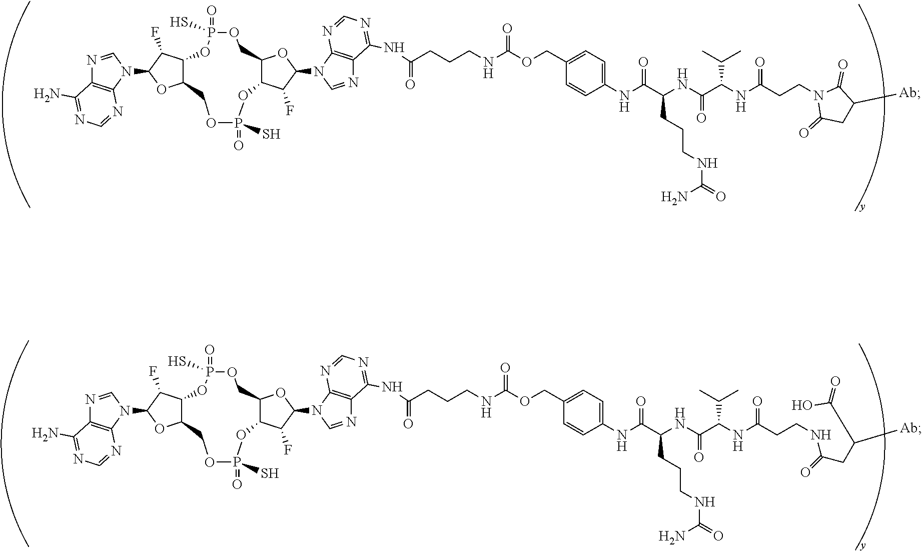

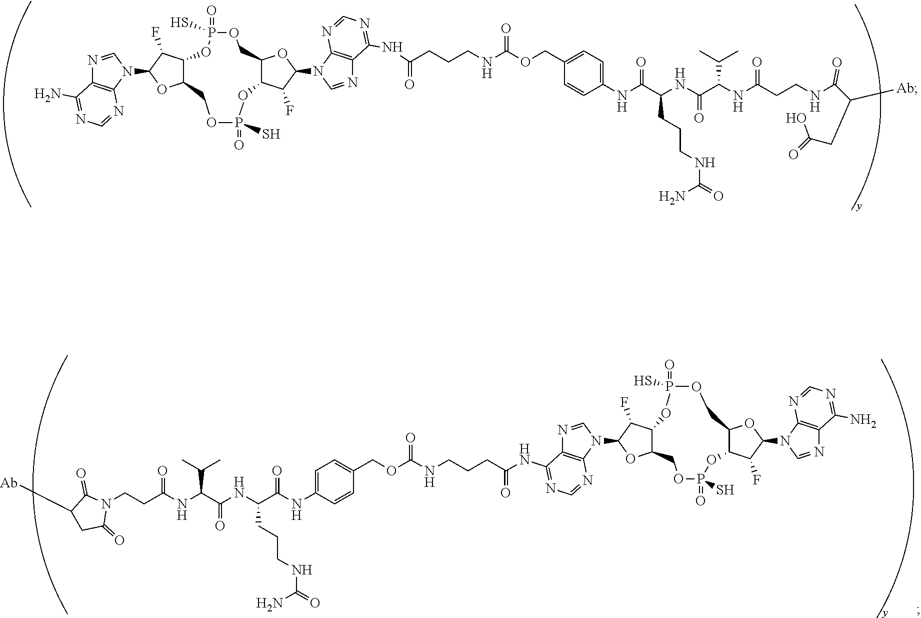

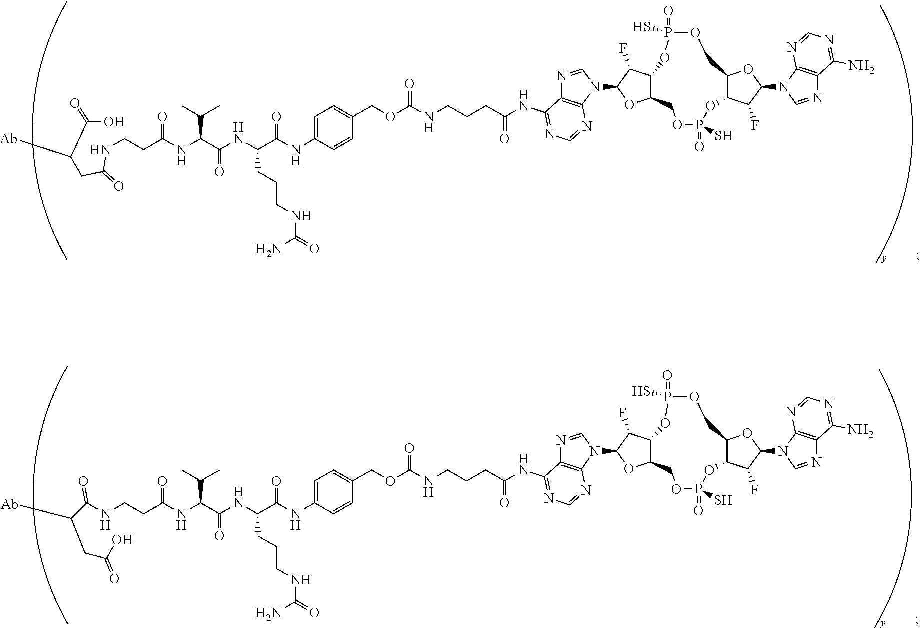

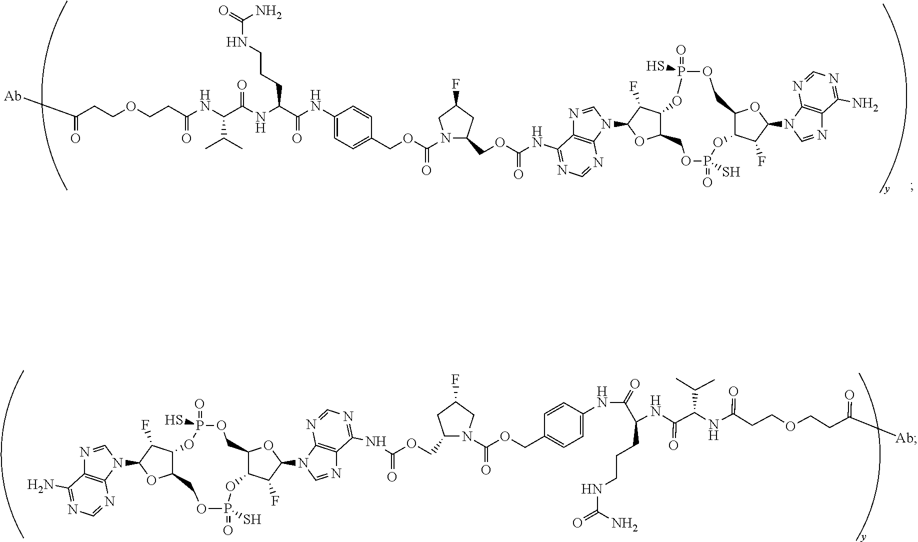

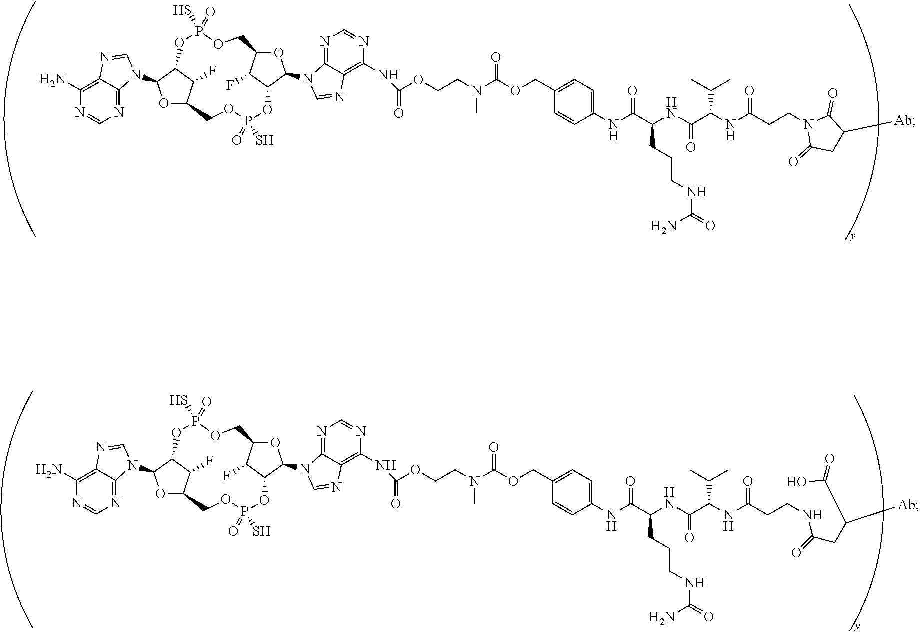

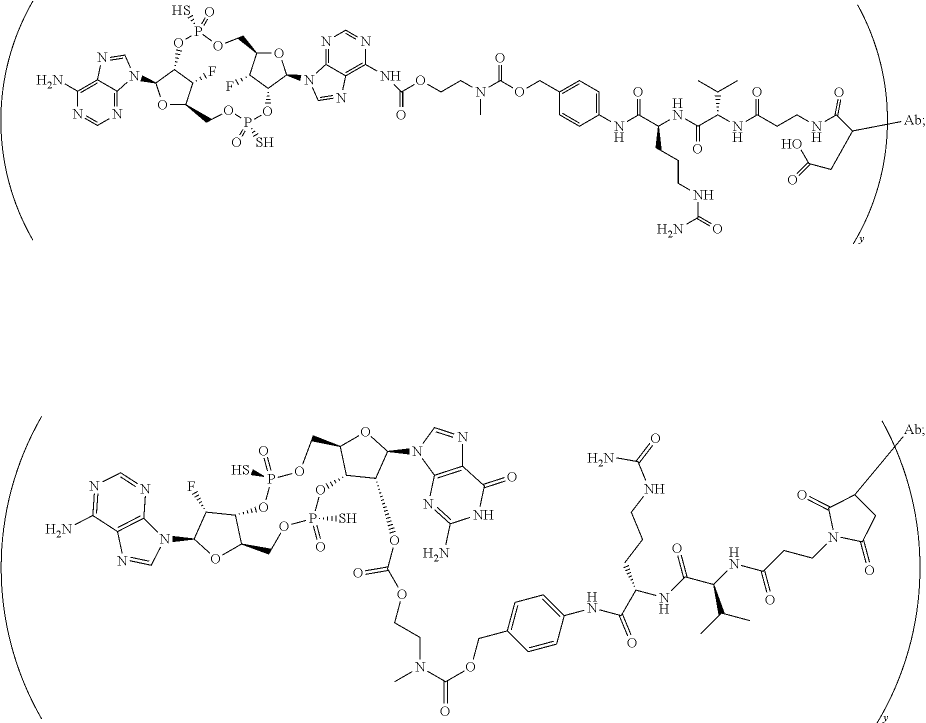

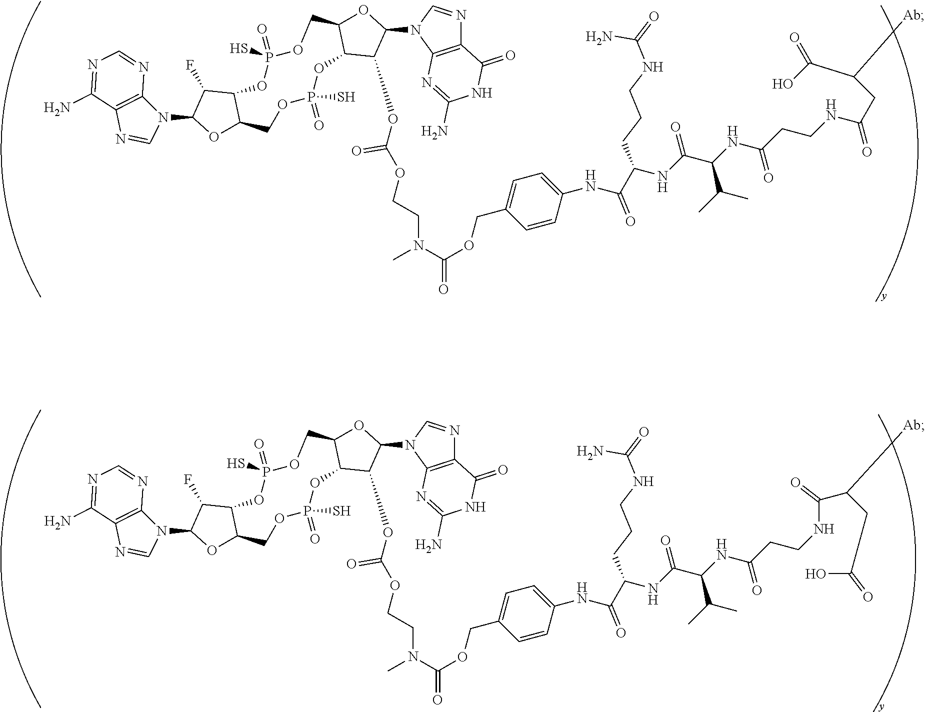

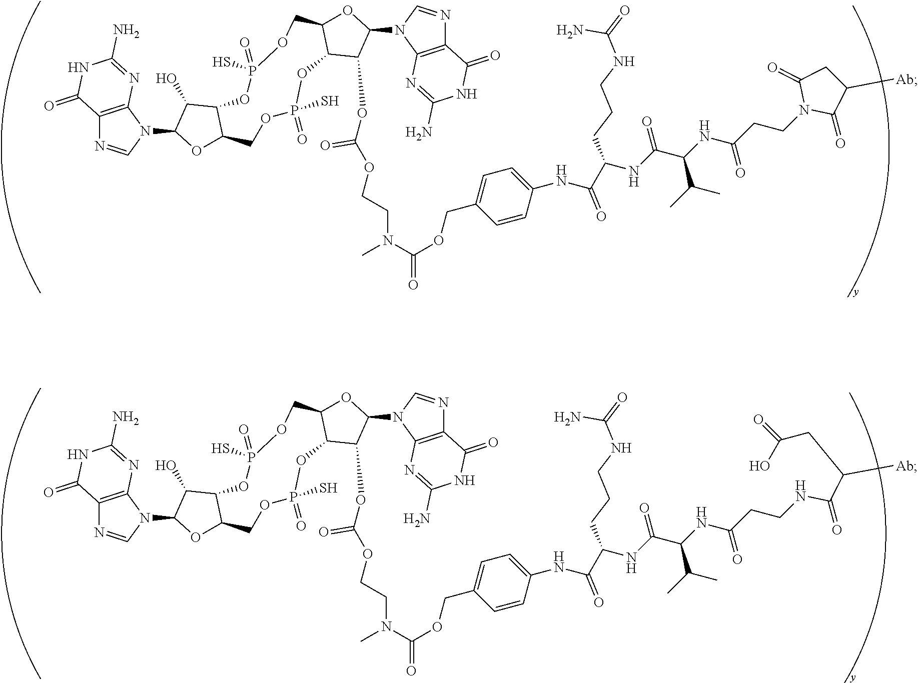

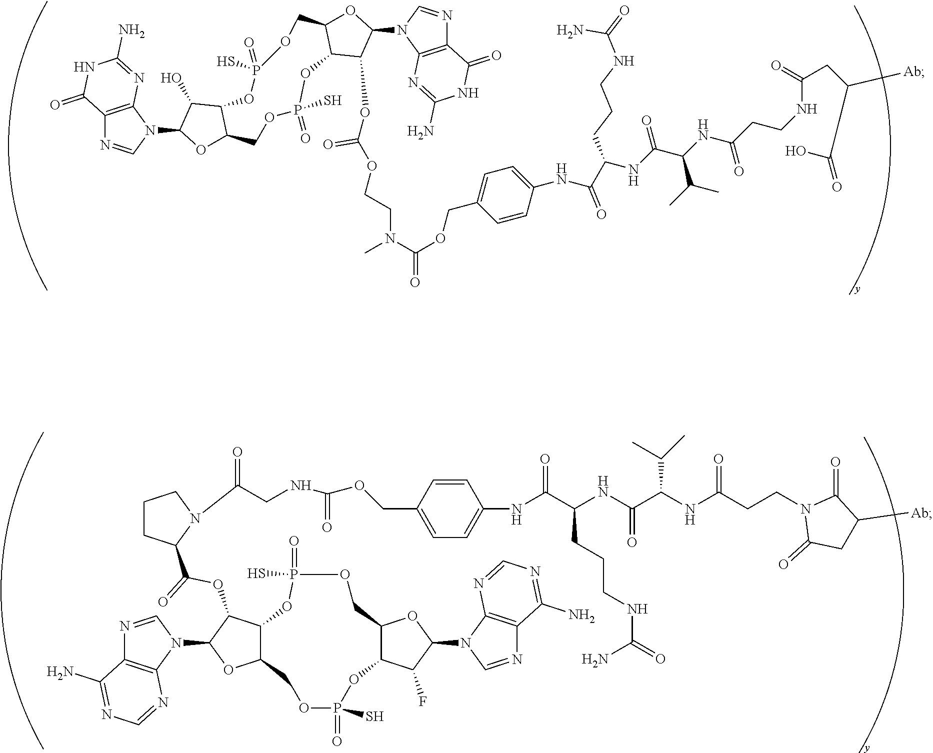

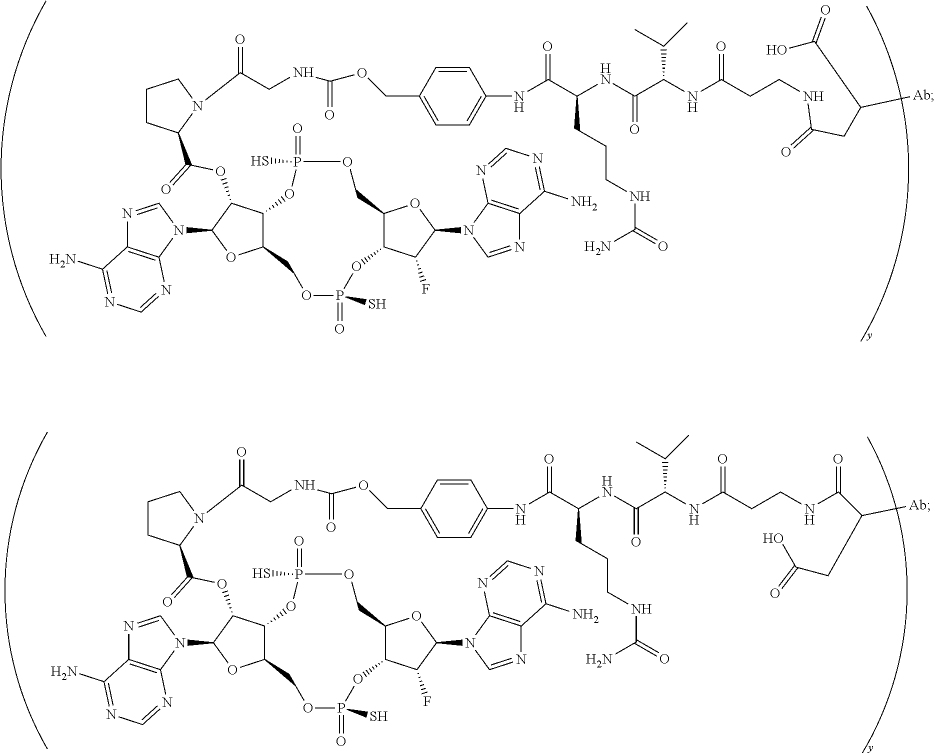

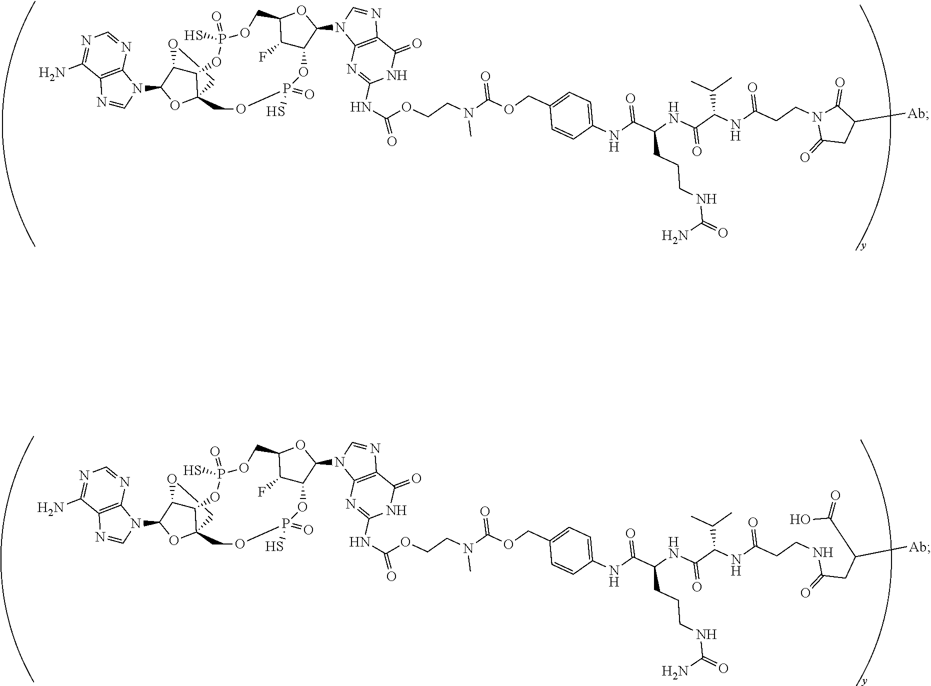

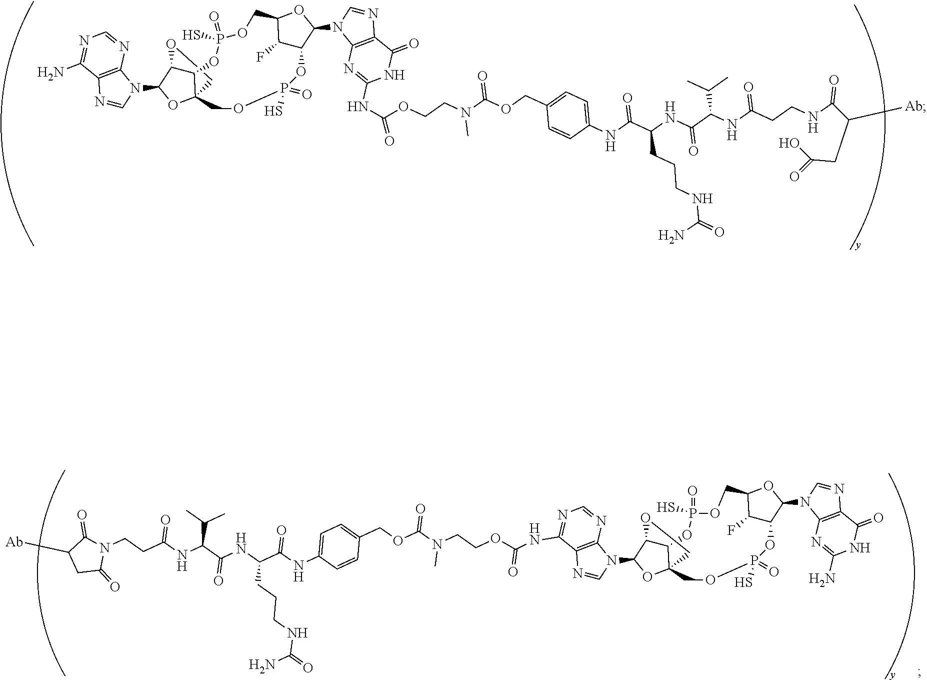

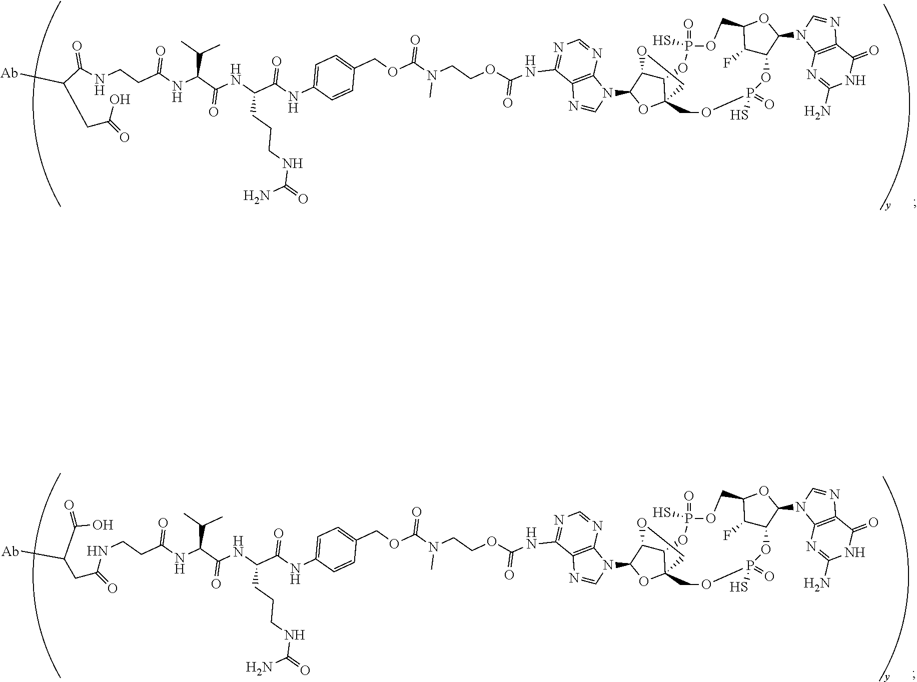

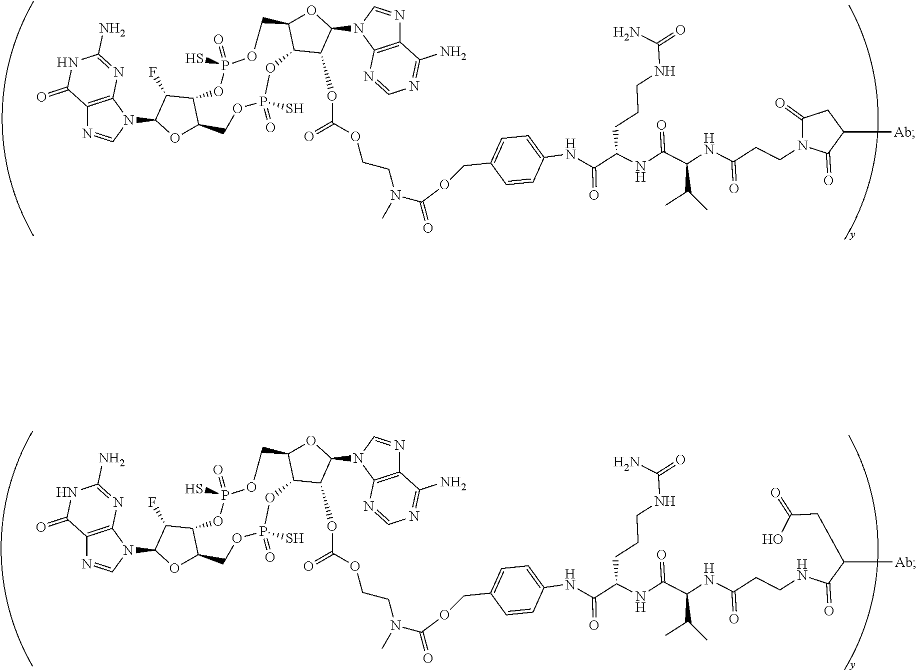

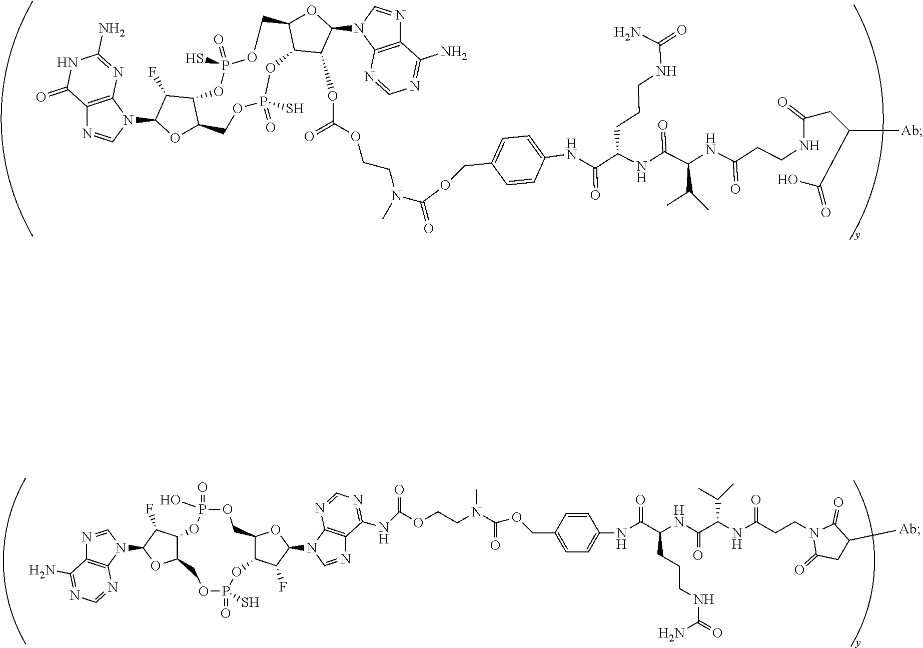

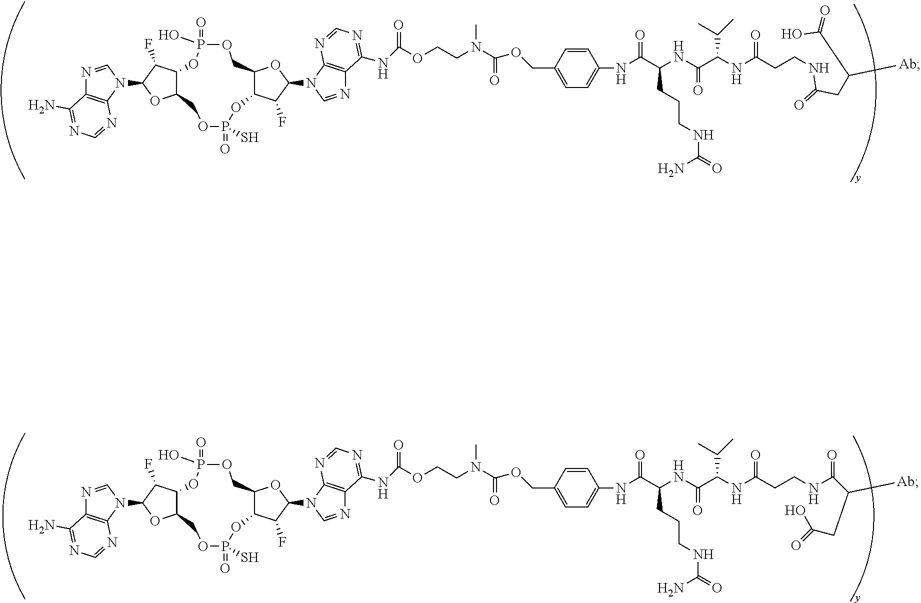

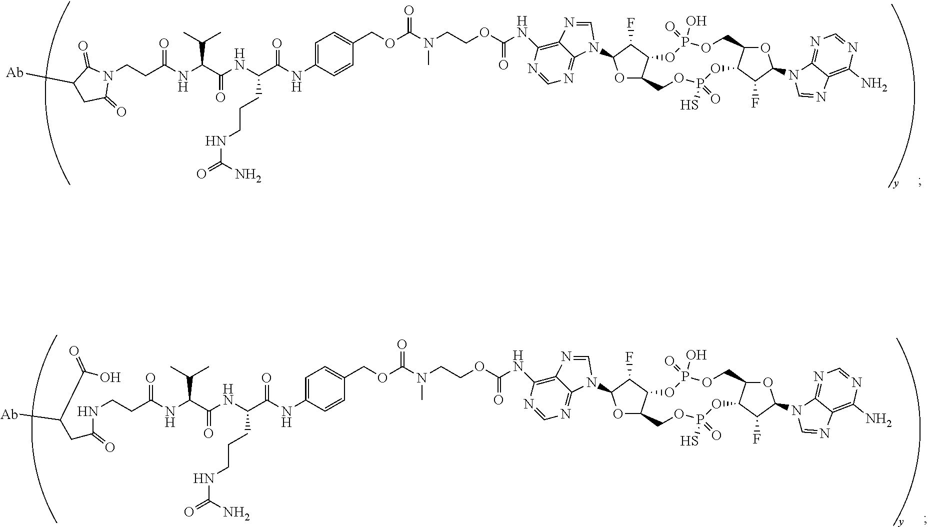

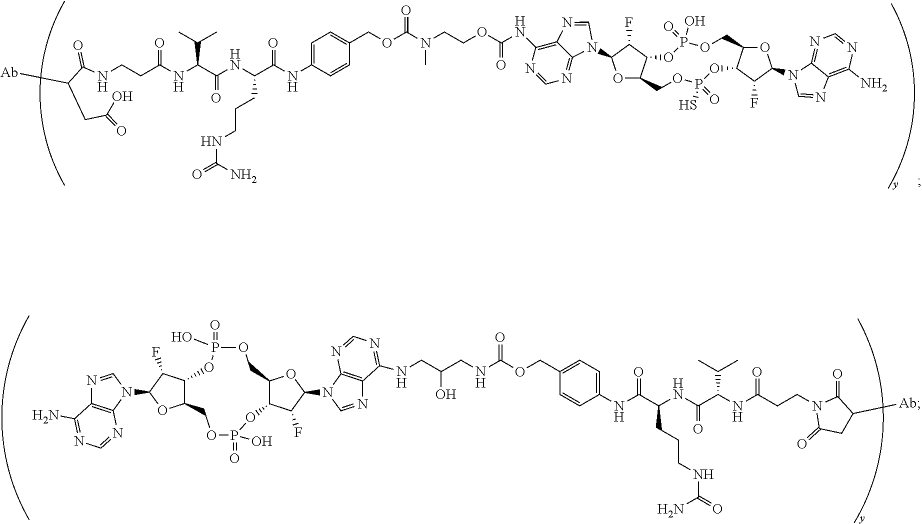

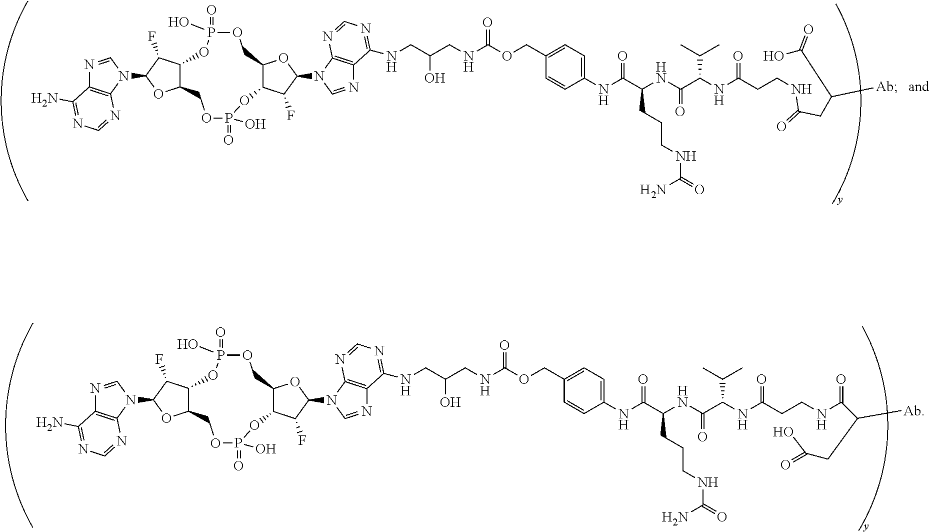

In some embodiments, the immunoconjugate has in vivo anti-tumor activity.

The present application also discloses a pharmaceutical composition comprising an immunconjugate as disclosed herein and a pharmaceutically acceptable excipient.

The present application also discloses an immunoconjugate as disclosed herein for use in combination with one or more additional therapeutic agents. In one embodiment, the additional therapeutic agent is selected from the group consisting of an inhibitor of a co-inhibitory molecule, an activator of a co-stimulatory molecule, a cytokine, an agent that reduces cytokine release syndrome (CRS), a chemotherapy, a targeted anti-cancer therapy, an oncolytic drug, a cytotoxic agent, an immune-based therapy, a vaccine, or a cell therapy. In another embodiment, the additional therapeutic agent is an inhibitor of a co-inhibitory molecule, an activator of a co-stimulatory molecule, or a cytokine, wherein:

(i) the co-inhibitory molecule is selected from Programmed death-1 (PD-1), Programmed death-ligand 1 (PD-L1), Lymphocyte activation gene-3 (LAG-3), or T-cell immunoglobulin domain and mucin domain 3 (TIM-3),
(ii) the co-stimulatory molecule is Glucocorticoid-induced TNFR-related protein (GITR), and
(iii) the cytokine is IL-15 complexed with a soluble form of IL-15 receptor alpha (IL-15Ra).

The present application also discloses a method of treating cancer comprising administering to a patient in need thereof a therapeutically effective amount of an immunconjugate, a pharmaceutical composition thereof, or a composition comprising an immunoconjugate in combination with one or more additional therapeutic agents, as disclosed herein.

The present application also discloses use of an immunconjugate, a pharmaceutical composition thereof, or a composition comprising an immunoconjugate in combination with one or more additional therapeutic agents, as disclosed herein for treatment of a cancer in a subject in need thereof.

In another embodiment, this application discloses an immunconjugate, a pharmaceutical composition thereof, or a composition comprising an immunoconjugate in combination with one or more additional therapeutic agents, as disclosed herein for use in the treatment of cancer.

In yet another embodiment, disclosed herein is the use an immunconjugate, a pharmaceutical composition thereof, or a composition comprising an immunoconjugate in combination with one or more additional therapeutic agents, as disclosed herein in the manufacture of a medicament for use in the treatment of cancer.

In some embodiments, the cancer is selected from sarcomas, adenocarcinomas, blastomas, carcinomas, liver cancer, lung cancer, non-small cell lung cancer, small cell lung cancer, breast cancer, lymphoid cancer, colon cancer, renal cancer, urothelial cancer, prostate cancer, cancer of the pharynx, rectal cancer, renal cell carcinoma, cancer of the small intestine, esophageal cancer, melanoma, bone cancer, pancreatic cancer, skin cancer, cancer of the head or neck, cutaneous or intraocular malignant melanoma, uterine cancer, ovarian cancer, colorectal cancer, cancer of the anal region, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, environmentally induced cancers including those induced by asbestos, leukemia, lymphoma, acute myelogenous leukemia (AML), acute lymphoid leukemia (ALL), chronic myelogenous leukemia (CML), chronic lymphoid leukemia (CLL), myelodysplastic syndromes, B-cell acute lymphoid leukemia (“BALL”), T-cell acute lymphoid leukemia (“TALL”), B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia, myelodysplastic syndrome, plasmablastic lymphoma, plasmacytoid dendritic cell neoplasm, and Waldenstrom macroglobulinemia.

In some embodiments, the immunoconjugate is administered to the subject intravenously, intratumorally, or subcutaneously.

The present application also discloses an immunconjugate, a pharmaceutical composition thereof, or a composition comprising an immunoconjugate in combination with one or more additional therapeutic agents, as disclosed herein for use as a medicament.

This application also discloses a method of manufacturing any of the immunoconjugates as disclosed herein comprising the steps of:

a) Reacting D and L to form L-(D)ₘ; and
b) Reacting L-(D)ₘ with Ab to form the immunoconjugate Ab-(L-(D)ₘ)ₙ (Formula (I)).

In another embodiment, this application discloses a compound having a structure selected from Formula (A), Formula (B), Formula (C), Formula (D), Formula (E), or Formula (F) or stereoisomers or pharmaceutically acceptable salts thereof,

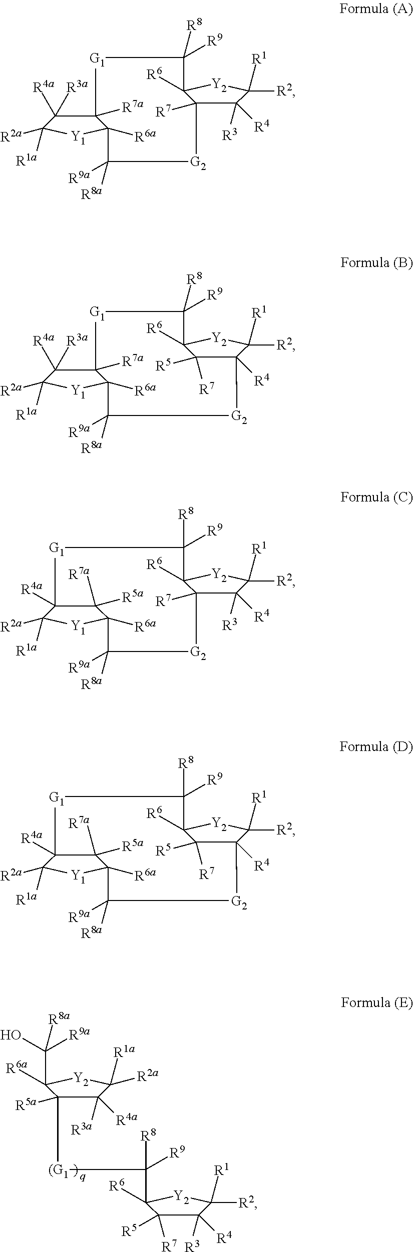

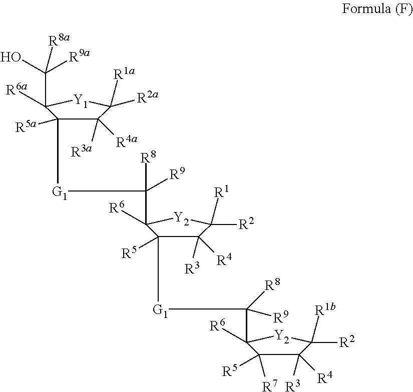

wherein:
each G₁ is independently selected from

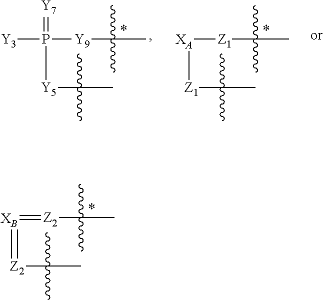

where the * of G₁ indicates the point of attachment to —CR⁸R⁹—;
X_{A} is C(═O)—, —C(═S)— or —C(═NR¹¹)— and each Z₁ is NR¹²;
X_{B} is C, and each Z₂ is N;

### G₂ is

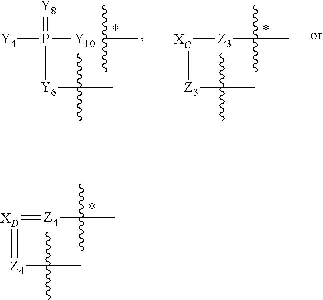

where the * of G₂ indicates the point of attachment to —CR^{8a}R^{9a}—;
X_{C} is C(═O)—, —C(═S)— or —C(═NR¹¹)— and each Z₃ is NR¹²;
X_{D} is C, and each Z₄ is N;
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₃ is OH, O⁻, OR¹⁰, N(R¹⁰)₂, SR¹⁰, SeH, Se⁻, BH₃, SH or S⁻;
Y₄ is OH, O⁻, OR¹⁰, N(R¹⁰)₂, SR¹⁰, SeH, Se⁻, BH₃, SH or S⁻;
Y₅ is —CH₂—, —NH—, —O— or —S;
Y₆ is —CH₂—, —NH—, —O— or —S;

### Y₇ is O or S;

### Y₈ is O or S;

Y₉ is —CH₂—, —NH—, —O— or —S;
Y₁₀ is —CH₂—, —NH—, —O— or —S;
Y₁₁ is —O—, —S—, —S(═O)—, —SO₂—, —CH₂—, or —CF₂—;
q is 1, 2 or 3;
R¹ is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R¹ is substituted with 0, 1, 2, 3 or 4 substituents independently selected from —NHL₁R¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═H(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1a} is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1a} is substituted with 0, 1, 2, 3 or 4 substituents independently selected from —NHL₁R¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1b} is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1b} is substituted with 0, 1, 2, 3 or 4 substituents independently selected from —NHL₁R¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
each R⁸ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁸ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R⁹ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁹ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁹ are substituted by 0, 1, 2 or 3 substituents independently selected from F, Cl, Br, I, OH, CN, and N₃;
R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
R^{3a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
R^{4a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
R^{5a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
R^{7a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
R^{8a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{8a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
R^{9a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{9a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{9a} are substituted by 0, 1, 2 or 3 substituents independently selected from F, Cl, Br, I, OH, CN, and N₃;
each R¹⁰ is independently selected from the group consisting of H, C₁-C₁₂alkyl, C₁-C₆heteroalkyl, —(CH₂CH₂O)ₙCH₂CH₂C(═O)OC₁-C₆alkyl, and

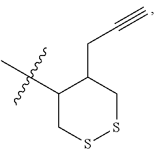 [SMILES: *C1CSSCC1CC#C]

wherein the C₁-C₁₂alkyl and C₁-C₆heteroalkyl of R¹⁰ is substituted by 0, 1, 2 or 3 substituents independently selected from —OH, C₁-C₁₂alkoxy, —S—C(═O)C₁-C₆alkyl, halo, —CN, C₁-C₁₂alkyl, —O-aryl, _O-heteroaryl, —O-cycloalkyl, oxo, cycloalkyl, heterocyclyl, aryl, or heteroaryl, —OC(O)OC₁-C₆alkyland C(O)OC₁-C₆alkyl, wherein each alkyl, cycloalkyl, heterocyclyl, aryl, and heteroaryl is substituted by 0, 1, 2 or 3 substituents independently selected from C₁-C₁₂ alkyl, O—C₁-C₁₂alkyl, C₁-C₁₂heteroalkyl, halo, CN, OH, oxo, aryl, heteroaryl, O-aryl, O-heteroaryl, —C(═O)C₁-C₁₂alkyl, —OC(═O)C₁-C₁₂alkyl, —C(═O)OC₁-C₁₂alkyl, —OC(═O)OC₁-C₁₂alkyl, —C(═O)N(R¹¹)—C₁-C₁₂alkyl, —N(R¹¹)C(═O)—C₁-C₁₂alkyl; —OC(═O)N(R¹¹)—C₁-C₁₂alkyl, —C(═O)-aryl, —C(═O)-heteroaryl, —OC(═O)-aryl, —C(═O)O-aryl, —OC(═O)-heteroaryl, —C(═O)O-heteroaryl, —C(═O)O-aryl, —C(═O)O-heteroaryl, —C(═O)N(R¹¹)-aryl, —C(═O)N(R¹¹)-heteroaryl, —N(R¹¹)C(O)-aryl, —N(R¹¹)₂C(O)-aryl, —N(R¹¹)C(O)-heteroaryl, and S(O)₂N(R¹¹)-aryl;
each R¹¹ is independently selected from H and C₁-C₆alkyl;
each R¹² is independently selected from H and C₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position;
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position;
optionally R⁸ and R⁹ are connected to form a C₁-C₆alkylene, C₂-C₆alkenylene, C₂-C₆alkynylene, and
optionally R^{8a} and R^{9a} are connected to form a C₁-C₆alkylene, C₂-C₆alkenylene, C₂-C₆alkynylene,
L₁ is —C(═O)O(CH₂)ₘNR¹¹C(═O)(CH₂)ₘ—**; —C(═O)O(CH₂)ₘNR¹¹C(═O)(CH₂)ₘO(CH₂)ₘ—**; —C(═O)O(CH₂)ₘNR¹¹C(═O)X₁X₂C)X₁X₂C(═O)(CH₂)ₘ—**; —C(═O)OC(R¹²)₂(CH₂)ₘNR¹¹C(═O)X₁X₂C(═O)(CH₂)ₘ—**; —C(═O)O(CH₂)ₘNR¹¹C(═O)X₁X₂C(═O)(CH₂)ₘO(CH₂)ₘ—**; —C(═O)O(CH₂)ₘNR¹¹C(═O)X₁X₂C(═O)(CH₂)ₘO(CH₂)ₘC(═O)—**; —C(═O)O(CH₂)ₘNR¹¹C(═O)X₄C(═O)NR¹¹ (CH₂)ₘNR¹¹C(═O)(CH₂)ₘO(CH₂)ₘ—**; —C(═O)O(CH₂)ₘNR¹¹C(═O)X₅C(═O)(CH₂)ₘNR¹¹C(═O)(CH₂)ₘ—**; —C(═O)X₄C(═O)NR¹¹ (CH₂)ₘNR¹¹C(═O)(CH₂)ₘO(CH₂)ₘ—**; —C(═O)(CH₂)ₘNR¹¹C(═O)X₁X₂C(═O)(CH₂)ₘ—**; —C(═O)(CH₂)ₘNR¹X₂C(═O)(CH₂)ₘ—**; —C(═O)O(CH₂)ₘX₆C(═O)X₁X₂C(═O)(CH₂)ₘ—**, —C(═O)(CH₂)ₘNR¹¹C(═O)((CH₂)ₘO)ₙ(CH₂)ₘ—**, —C(═O)O(CH₂)ₘX₆C(═O) (CH₂)ₘ—**, —C(═O)O(CH₂)ₘX₆C(═O)(CH₂)ₘO(CH₂)ₘ—**, —C(═O)O(CH₂)ₘX₆C(═O)X₁X₂C(═O)(CH₂)ₘ—**, —C(═O)O(CH₂)ₘX₆C(═O)X₁X₂C(═O)(CH₂)ₘO(CH₂)ₘ—**, —C(═O)O(CH₂)ₘX₆C(═O)X₁X₂C(═O)(CH₂)ₘO(CH₂)ₘC(═O)—**; —C(═O)O(CH₂)ₘX₆C(═O)X₄C(═O)NR¹¹(CH₂)ₘNR¹¹C(═O)(CH₂)ₘO(CH₂)ₘ—**, —C(═O)X₄C(═O)X₆(CH₂)ₘNR¹¹C(═O)(CH₂)ₘO(CH₂)ₘ—**, —C(═O)(CH₂)ₘX₆C(═O)X₁X₂C(═O)(CH₂)ₘ**, —C(═O)O((CH₂)ₘO)ₙ(CH₂)ₘNR¹¹C(═O)X₅C(═O)(CH₂)ₘ—**; —C(═O)O((CH₂)ₘO)ₙ(CH₂)ₘNR¹¹C(═O)X₅C(═O)(CH₂)ₘNR¹¹C(═O)(CH₂)ₘ—**; —C(═O)O((CH₂)ₘO)ₙ(CH₂)ₘNR¹¹C(═O)X₅C(═O)(CH₂)ₘX₃(CH₂)ₘ—**; —C(═O)O((CH₂)ₘO)ₙ(CH₂)ₘNR¹¹C(═O)X₅C(═O)((CH₂)ₘO)ₙ(CH₂)ₘ—**; —C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘ—**; —C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)O((CH₂)ₘO)ₙ(CH₂)ₘNR¹¹C(═O))X₅C(═O) ((CH₂)ₘO)ₙ(CH₂)ₘX₃(CH₂)ₘ—**; —C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘ—**; —C(═O)O((CH₂)ₘO)ₙ(CH₂)ₘNR¹¹C(═O)X₅C(═O)(CH₂)ₘNR¹¹C(═O) ((CH₂)ₘO)ₙ(CH₂)ₘX₃(CH₂)ₘ—**; —C(═O)O((CH₂)ₘO)ₙ(CH₂)ₘNR¹¹C(═O)X₅(CH₂)ₘX₃(CH₂)ₘ—**; —C(═O)O((CH₂)ₘO)ₙ(CH₂)ₘNR¹¹C(═O)X₅((CH₂)ₘO)ₙ(CH₂)ₘ—**; —C(═O)O((CH₂)ₘO)ₙ(CH₂)ₘNR¹¹C(═O)X₅((CH₂)ₘO)ₙ(CH₂)ₘNR¹¹C(═O)(CH₂)ₘ—**; —C(═O)O((CH₂)ₘO)ₙ(CH₂)ₘNR¹¹C(═O)X₅((CH₂)ₘO)ₙ(CH₂)ₘNR¹¹C(═O)(CH₂)ₘX(CH₂)ₘ—**; —C(═O)O((CH₂)ₘO)ₙ(CH₂)ₘNR¹¹C(═O)X₅((CH₂)ₘO)ₙ(CH₂)ₘX₃(CH₂)ₘ—**; —C(═O)O((CH₂)ₘO)ₙ(CH₂)ₘNR¹¹C(═O)X₅(CH₂)ₘNR¹¹((CH₂)ₘO)ₙ(CH₂)ₘ—**; —C(═O)O((CH₂)ₘO)ₙ(CH₂)ₘNR¹¹C(═O)X₅C(═O)(CH₂)ₘNR¹¹ ((CH₂)ₘO)ₙ(CH₂)ₘX₃(CH₂)ₘ**; —C(═O)O((CH₂)ₘO)ₙ(CH₂)ₘNR¹¹C(═O)X₅(CH₂)ₘ—**; —C(═O)O((CH₂)ₘO)ₙ(CH₂)ₘNR¹¹C(═O)X₅C(═O)((CH₂)ₘO)ₙ(CH₂)ₘ—**; —C(═O)O((CH₂)ₘO)ₙ(CH₂)ₘNR¹¹C(═O)X₅(CH₂)ₘX₃(CH₂)ₘ—**; C(═O)O(CH)ₘ**; —C(═O)O((CH₂)ₘO)ₙ(CH₂)ₘ—**; C(═O)O(CH₂)ₘNR¹¹ (CH₂)ₘ—**; —C(═O)O(CH₂)ₘNR¹¹ (CH₂)ₘC(═O)X₂X_{C}(═O)**; —C(═O)O(CH₂)ₘX₃(CH₂)ₘ—**; C(═O)O(CH₂)ₘX₆C(═O)X₁X₂C(═O)((CH₂)ₘO)ₙ(CH₂)ₘ**; —C(═O)O((CH₂)ₘO)ₙ(CH₂)ₘX₃(CH₂)ₘ—**; —C(═O)O((CH₂)ₘO)ₙ(CH₂)ₘNR¹¹C(═O)(CH₂)ₘ—**; —C(═O)O(CH₂)ₘNR¹¹C(═O(CH₂)ₘX₃(CH₂)ₘ—**; —C(═O)O((CH₂)ₘO)ₙ(CH₂)ₘNR¹¹C(═O)(CH₂)ₘX₃(CH₂)ₘ—**; —C(═O)O((CH₂)ₘO)ₙX₃(CH₂)ₘ—**; C(═O)O((CH₂)ₘO)ₙ(CH₂)ₘX₃(CH₂)ₘ—**; —C(═O)O((CH₂)ₘO)ₙ(CH₂)ₘC(═O)NR¹¹(CH₂)ₘ—**; C(═O)O(CH₂)ₘC(R¹²)₂—**; —C(═O)OCH₂)ₘC(R¹²)₂SS(CH₂)ₘNR¹¹C(═O)(CH₂)ₘ—**; —C(═O)O(CH₂)ₘC(═O)NR¹¹(CH₂)ₘ—**; C(═O)(CH₂)ₘ—**; C(═O)((CH₂)ₘO)ₙ(CH₂)ₘ—**; —C(═O)(CH₂)ₘNR¹¹(CH₂)ₘ—**; C(═O)(CH₂)ₘNR¹¹ (CH₂)ₘC(═O)X₂X₁C(═O)—**; —C(═O)(CH₂)ₘX₃(CH₂)ₘ—**; C(═O)((CH₂)ₘO)ₙ(CH₂)ₘX₃(CH₂)ₘ—**; —C(═O)(CH₂)ₘNR¹¹C(═O)(CH₂)ₘ—**; C(═O)((CH₂)ₘO)ₙ(CH₂)ₘNR¹¹C(═O)(CH₂)ₘ—**; —C(═O)(CH₂)ₘNR¹¹C(═O(CH₂)ₘX₃(CH₂)ₘ—**; (CH₂)ₘNR¹¹C(═O)X₁X₂C(═O)(CH₂)ₘ—**; —(CH₂)ₘ(CHOH)(CH₂)ₘNR¹¹C(═O)X₁X₂C(═O)(CH₂)ₘ**; —C(═O)((CH₂)ₘO)ₙ(CH₂)ₘNR¹¹C(═O)(CH₂)ₘX₃(CH₂)ₘ—**; C(═O)((CH₂)ₘO)ₙX₃(CH₂)ₘ—**; —C(═O)((CH₂)ₘO)ₙ(CH₂)ₘX₃(CH₂)ₘ—**; C(═O)((CH₂)ₘO)ₙ(CH₂)ₘC(═O)NR¹¹(CH₂)ₘ—**; —C(═O)(CH₂)ₘC(R¹²)₂—**; C(═O)((CH₂)O)(CH)NR¹¹C(═O)X₅C(═O)(CH₂)ₘ**; —C(═O)((CH₂)ₘO)ₙ(CH₂)ₘNR¹¹C(═O)X₅C(═O)(CH₂)ₘNR¹¹C(═O)(CH₂)ₘ—**; —C(═O)((CH₂)ₘO)ₙ(CH₂)ₘNR¹¹C(═O)X₅C(═O)(CH₂)ₘX(CH₂)ₘ—**; —C(═O)((CH₂)ₘO)ₙ(CH₂)ₘNR¹¹C(═O)X₅C(═O)((CH₂)ₘO)ₙ(CH₂)ₘ—**; —C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘ—**; —C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)((CH₂)ₘO)ₙ(CH₂)ₘNR¹¹C(═O))X₅C(═O)((CH₂)ₘO)ₙ(CH₂)ₘX₃(CH₂)ₘ—**; —C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘ—**; —C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘX₃(CH₂)ₘ—**; —C(═O)((CH₂)ₘO)ₙ(CH₂)ₘNR¹¹C(═O)X₅((CH₂)ₘO)ₙ(CH₂)ₘ—**; —C(═O)((CH₂)ₘO)ₙ(CH₂)ₘNR¹¹C(═O)X₅((CH₂)ₘO)ₙ(CH₂)ₘNR¹¹C(═O)(CH₂)ₘ—**; —C(═O)((CH₂)ₘO)ₙ(CH₂)ₘNR¹¹C(═O)X₅((CH₂)ₘO)ₙ(CH₂)ₘNR¹¹C(═O)(CH₂)ₘX₃(CH₂)ₘ—**; —C(═O)((CH₂)ₘO)ₙ(CH₂)ₘNR¹¹C(═O)X₅((CH₂)ₘO)ₙ(CH₂)ₘX₃(CH₂)ₘ—**; —C(═O)((CH₂)ₘO)ₙ(CH₂)ₘNR¹¹C(═O)X(CH₂)ₘNR¹¹((CH₂)ₘO)ₙ(CH₂)ₘ—**; —C(═O)((CH₂)ₘO)ₙ(CH₂)ₘNR¹¹C(═O)X₅C(═O)(CH₂)ₘNR¹¹ ((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘ—**; —C(═O) ((CH₂)ₘO)ₙ(CH₂)ₘNR¹¹C(═O)X₅C(═O)((CH₂)ₘO)ₙ(CH₂)ₘ—**; —C(═O)((CH₂)ₘO)ₙ(CH₂)ₘNR¹¹C(═O)X₅(CH₂)ₘX₃(CH₂)ₘ—**; —C(═O)(CH₂)ₘC(R¹²)₂SS(CH₂)ₘNR¹¹C(═O)(CH₂)ₘ—**; C(═O)(CH₂)ₘC(═O)NR¹¹(CH₂)ₘ—**; —C(═O)X₁X₂C(═O)(CH₂)ₘ—**; C(═O)X₁X₂C(═O)(CH₂)ₘNR¹¹C(═O)(CH₂)ₘ—**; —C(═O)X₁X₂C(═O)(CH₂)ₘX₃(CH₂)ₘ—**; C(═O)X₁X₂C(═O)((CH₂)ₘO)ₙ(CH₂)ₘ—**; —C(═O)X₁X₂C(═O)((CH₂)ₘO)ₙ(CH₂)ₘNR¹¹C(═O)(CH₂)ₘ—**; —C(═O)X₁X₂C(═O)((CH₂)ₘO)ₙ(CH₂)ₘNR¹¹C(═O)(CH₂)ₘX₃(CH₂)ₘ—**; —C(═O)X₁X₂C(═O) ((CH₂)ₘO)ₙ(CH₂)ₘX₃(CH₂)ₘ—**; —C(═O)X₁X₂C(═O)(CH₂)ₘNR¹¹C(═O)((CH₂)ₘO)ₙ(CH₂)ₘ—**; —C(═O)X₁X₂C(═O)(CH₂)ₘNR¹¹C(═O)((CH₂)ₘO)ₙ(CH₂)ₘX₃(CH₂)ₘ—**; —C(═O)X₁X₂(CH₂)ₘX₃(CH₂)ₘ—**; C(═O)X₁X₂((CH₂)ₘO)ₙ(CH₂)ₘ—**; —C(═O)X₁X₂((CH₂)ₘO)ₙ(CH₂)ₘNR¹¹C(═O)(CH₂)ₘ—**; —C(═O)X₁X₂((CH₂)ₘO)ₙ(CH₂)ₘNR¹¹C(═O)(CH₂)ₘX₃(CH₂)ₘ—**; —C(═O)X₁X₂((CH₂)ₘO)ₙ(CH₂)ₘX₃(CH₂)ₘ—**; —C(═O)X₁X₂(CH₂)ₘNR¹¹ ((CH₂)ₘO)ₙ(CH₂)ₘ—**; —C(═O)X₁X₂C(═O)(CH₂)ₘNR¹¹ ((CH₂)ₘO)ₙ(CH₂)ₘX₃(CH₂)ₘ—**; —C(═O)NR¹¹(CH₂)ₘ—**; C(═O)NR(CH₂)ₘX₃(CH₂)ₘ—**; —C(═O)NR¹¹(CH₂)ₘNR¹¹C(═O)X₁X₂C(═O)(CH₂)ₘ—**; —C(═O)NR¹¹(CH₂)ₘNR¹¹C(═O)O(CH₂)ₘ—**; C(═O)NR¹¹(CH₂)ₘNR¹¹C(═O)X₁X₂—**; —C(═O)NR¹¹ (CH₂)ₘNR¹¹C(═O)X₅; —C(═O)NR¹¹(CH₂)ₘNR¹¹C(═O)(CH₂)ₘX₅(CH₂)ₘ—**; —C(═O)X₁C(═O)NR(CH₂)ₘX₅(CH₂)ₘ—**; —C(═O)X₄C(═O)(CH₂)ₘNR¹¹C(═O)X₁X₂C(═O)(CH₂)ₘ—**; —C(═O)NR¹¹(CH₂)ₘNR¹¹C(═O)(CH₂)ₘ—**; C(═O)NR¹¹(CH₂)ₘNR¹¹C(═O)(CH₂)ₘO(CH₂)ₘ—**; —C(═O)NR¹¹(CH₂)ₘNR¹¹C(═O)X₁X₂C(═O)(CH₂)ₘO(CH₂)ₘ—**; —C(═O)NR¹¹(CH₂)ₘNR¹¹C(═O)X₁X₂C(═O)(CH₂)ₘO(CH₂)ₘC(═O)**; —C(═O)NR¹¹(CH₂)ₘNR¹¹C(═O)X₄C(═O)NR¹¹(CH₂)ₘNR¹¹C(═O)(CH₂)ₘO(CH₂)ₘ—**; —C(═O)NR¹¹(CH₂)ₘNR¹¹C(═O)X₁X₂C(═O)(CH₂)ₘ—**; —C(═O)NR¹¹(CH₂)ₘNR¹¹C(═O)X₅C(═O)(CH₂)ₘ—**; —C(═O)NR¹¹(CH₂)ₘNR¹¹C(═O)X₅C(═O)(CH₂)ₘNR¹¹C(═O)(CH₂)ₘ—**; —C(═O)NR¹¹(CH₂)ₘNR¹¹C(═O)X₅C(═O)(CH₂)ₘX₃(CH₂)ₘ**; —C(═O)NR¹¹(CH₂)ₘNR¹¹C(═O)X_{C}(═O)((CH₂)ₘO)ₙ(CH₂)ₘ—**; —C(═O)NR¹¹(CH₂)ₘNR¹¹C(═O)X₅C(═O)((CH₂)ₘO)ₙ(CH₂)ₘNR¹¹C(═O)(CH₂)ₘ—**; —C(═O)NR¹¹(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)NR¹¹(CH₂)ₘNR^{1l}C(═O)X₅C(═O)((CH₂)ₘO)ₙ(CH₂)ₘX₃(CH₂)ₘ—**; —C(═O)NR¹¹ (CH₂)ₘNR¹¹C(═O)X₅C(═O)(CH₂)ₘNR¹¹C(═O)((CH₂)ₘO)ₙ(CH₂)ₘ—**; —C(═O)NR¹¹ (CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O)NR¹¹(CH₂)ₘNR¹¹C(═O)X₅(CH₂)ₘX₃(CH₂)ₘ—**; —C(═O)NR¹¹(CH₂)ₘNR¹¹C(═O)X₅((CH₂)ₘO)ₙ(CH₂)ₘ—**; —C(═O)NR¹¹(CH₂)ₘNR¹¹C(═O)X₅((CH₂)ₘO)ₙ(CH₂)ₘNR¹¹C(═O)(CH₂)ₘ—**; —C(═O)NR¹¹(CH₂)ₘNR¹¹C(═O)X₅((CH₂)ₘO)ₙ(CH₂)ₘNR¹¹C(═O)(CH₂)ₘX₃(CH₂)ₘ—**; —C(═O)NR¹¹(CH₂)ₘNR¹¹C(═O)X₅((CH₂)ₘO)ₙ(CH₂)ₘX₃(CH₂)ₘ—**; —C(═O)NR¹¹(CH₂)ₘNR¹¹C(═O)X₅(CH₂)ₘNR¹¹((CH₂)ₘO)ₙ(CH₂)ₘ—**; —C(═O)NR¹¹(CH₂)ₘNR¹¹C(═O)X₅C(═O)(CH₂)ₘNR¹¹((CH₂)ₘO)ₙ(CH₂)ₘX₃(CH₂)ₘ**; —C(═O)NR¹¹(CH₂)ₘNR¹¹C(═O)X(CH₂)ₘ—**; —C(═O)NR¹¹(CH₂)ₘNR¹¹C(═O)X₅C(═O)((CH₂)ₘO)ₙ(CH₂)ₘ—**; —C(═O)NR(CH₂)ₘNR¹¹C(═O)X(CH₂)ₘX₃(CH₂)ₘ—**; —C(═O)X C(═O)NR¹¹(CH₂)ₘNR¹¹C(═O)(CH₂)ₘ—**; —C(═O)X₁C(═O)NR¹¹(CH₂)ₘX₃(CH₂)ₘ—**; C(═O)NR¹¹(CH₂)ₘNR¹¹C(═O)(CH₂)ₘ—**; —C(═O)NR¹¹(CH₂)ₘNR¹¹C(═O)(CH₂)ₘX₃(CH₂)ₘ—**; C(═O)NR¹¹(CH₂)ₘNR¹¹C(═O)—**; —C(═O)X₁X₂(CH₂)ₘ—**; C(═O)X₁X₂C(═O)((CH₂)ₘO)ₙ(CH₂)ₘ—**. —C(═O)X₁X₂(CH₂)ₘX₃(CH₂)ₘ—**; —C(═O)NR¹¹(CH₂)ₘX₃(CH₂)ₘ—**; —C(═O)NR¹¹ ((CH₂)ₘO)ₙ(CH₂)ₘX₃(CH₂)ₘ—**; —C(═O)X₁X₂C(═O) ((CH₂)ₘO)ₙ(CH₂)ₘ—**; —C(═O)X₁X₂C(═O)(CH₂)ₘ—**; —C(═O)X₁C(═O)(CH₂)ₘNR¹¹C(═O)(CH₂)ₘ—**; and C(═O)X₁C(═O)(CH₂)ₘNR¹¹C(═O)((CH₂)ₘO)ₙ(CH₂)ₘ—**;
where the ** of L₁ indicates the point of attachment to R¹⁵;

### R¹⁵ is

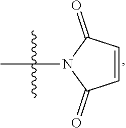 [SMILES: *N1C(=O)C=CC1=O]

—ONH₂, —NH₂,

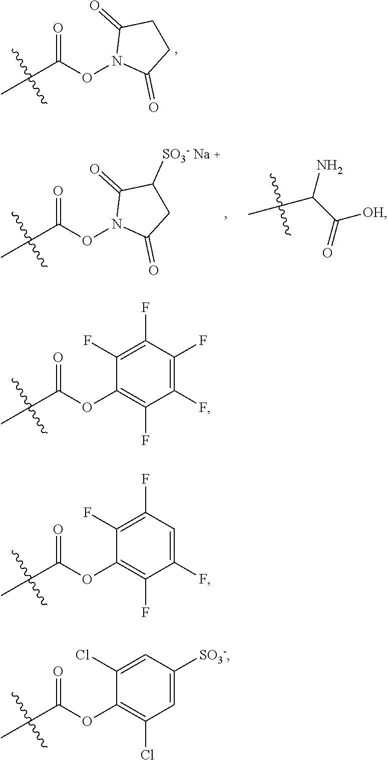

### —N₃,

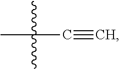 [SMILES: *C#C]

—SH, —SR¹², —SSR¹⁷, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I,

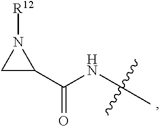

C(O)NHNH₂,

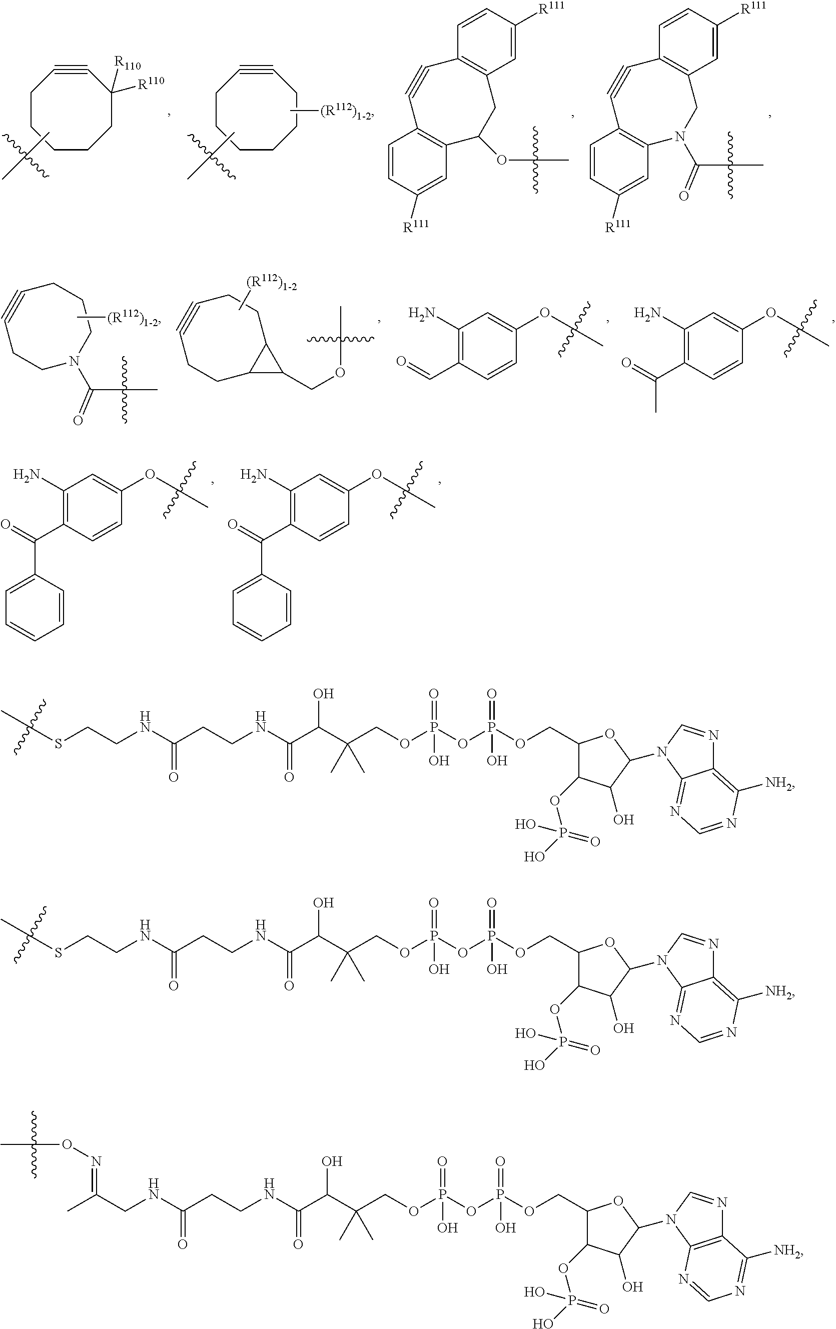

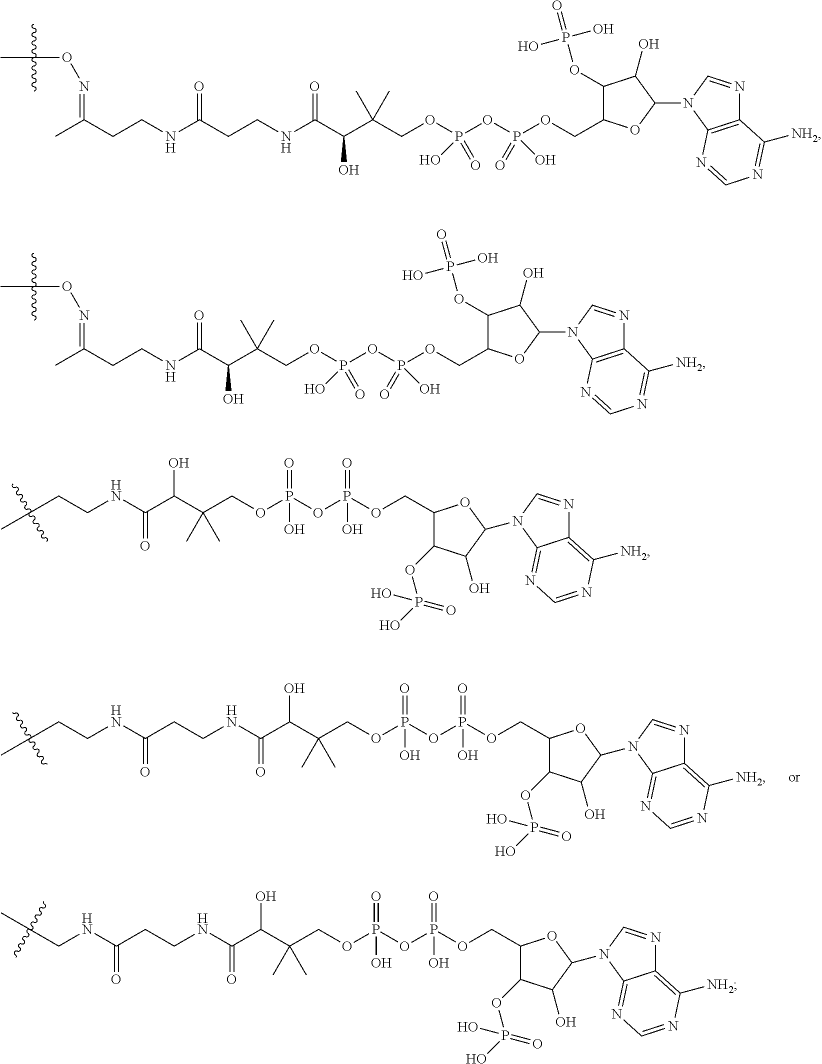 [SMILES: *O/N=C(\C)CCNC(=O)CCNC(=O)[C@H](O)C(C)(C)COP(=O)(O)OP(=O)(O)OCC1OC(n2cnc3c(N)ncnc32)C(O)C1OP(=O)(O)O ; *O/N=C(\C)CCNC(=O)[C@H](O)C(C)(C)COP(=O)(O)OP(=O)(O)OCC1OC(n2cnc3c(N)ncnc32)C(O)C1OP(=O)(O)O ; *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

### X₁ is

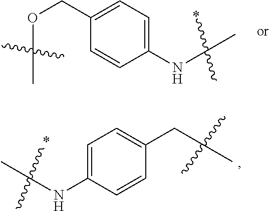 [SMILES: *Nc1ccc(CO*)cc1 ; *Cc1ccc(N*)cc1]

where the * of X₁ indicates the point of attachment to X₂;
X₂ is selected from

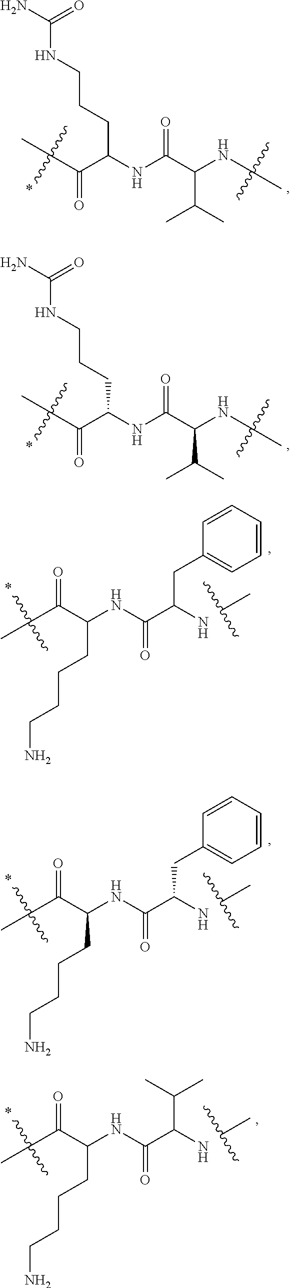

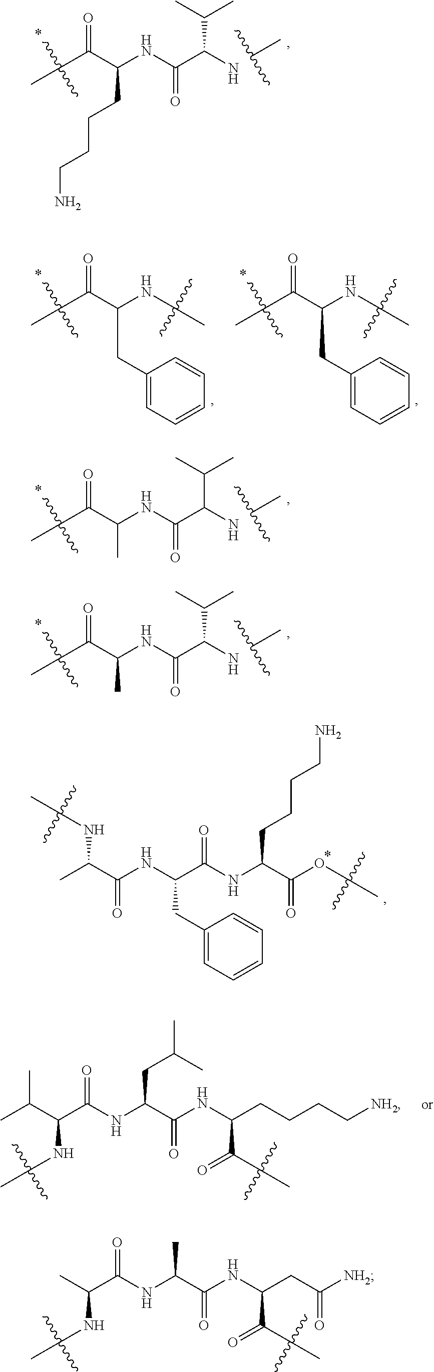

where the * of X₂ indicates the point of attachment to X₁ or to NR¹¹;

### X₃ is

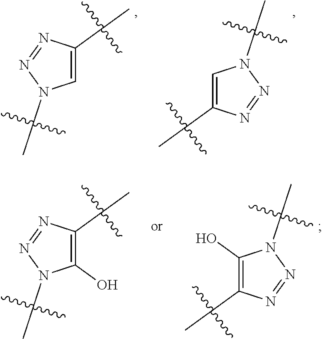 [SMILES: *c1cn(*)nn1 ; *c1cn(*)nn1 ; *c1nnn(*)c1O ; *c1nnn(*)c1O]

X₄ is —O(CH₂)ₙSSC(R¹²)₂(CH₂)ₙ— or —(CH₂)ₙC(R¹²)₂SS(CH₂)ₙO—;

### X₅ is

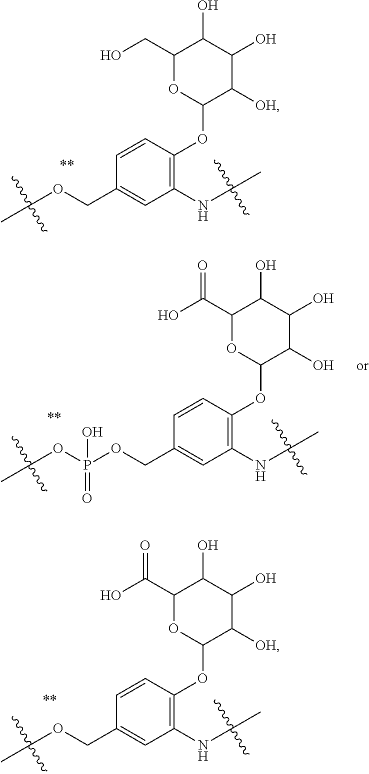 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the ** of X₅ indicates orientation toward R¹⁵;

### X₆ is

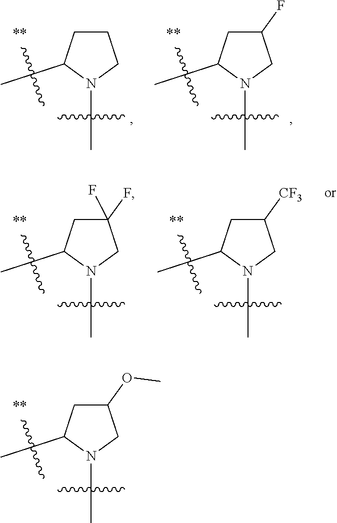

or, where the ** of X₆ indicates orientation toward R¹⁵;
R¹⁷ is 2-pyridyl or 4-pyridyl;
each R¹¹ is independently selected from H and C₁-C₆alkyl;
each R¹² is independently selected from H and C₁-C₆alkyl;
each m is independently selected from 1, 2, 3, 4, 5, 6, 7, 8, 9 and 10; and
each n is independently selected from 1, 2, 3, 4, 5, 6, 7, 8, 9, 10, 11, 12, 13, 14, 15, 16, 17 and 18.
each R¹¹⁰ is independently selected from H, C₁-C₆alkyl, F, Cl, and —OH;
each R¹¹¹ is independently selected from H, C₁-C₆alkyl, F, Cl, —NH₂, —OCH₃, —OCH₂CH₃, —N(CH₃)₂, —CN, —NO₂ and —OH;
each R¹¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
and provided at least one of R¹, R^{1a} or R^{1b} is substituted with —NHL₁R¹⁵, or at least one of R³, R⁴, R⁵, R⁷, R^{3a}, R^{4a}, R^{5a} or R^{7a} is —OL₁R¹⁵.

In some embodiments L₁ is —C(═O)O(CH₂)ₘNR¹¹C(═O)(CH₂)ₘ—**; —C(═O)O(CH₂)ₘNR¹¹C(═O)(CH₂)ₘO(CH₂)ₘ—**; —C(═O)O(CH₂)ₘNR¹¹C(═O)X₁X₂C(═O)(CH₂)ₘ—**; —C(═O)OC(R¹²)₂(CH₂)ₘNR¹¹C(═O)X₁X₂C(═O)(CH₂)ₘ—**; —C(═O)O(CH₂)ₘNR⁸C(═O)X₁X₂C(═O)(CH₂)ₘO(CH₂)ₘ—**; —C(═O)O(CH₂)ₘNR¹¹C(═O)X₁X₂C(═O)(CH₂)ₘO(CH₂)ₘC(═O)—**; —C(═O)O(CH₂)ₘNR¹¹C(═O)X₄C(═O)NR¹¹ (CH₂)ₘNR¹¹C(═O)(CH₂)ₘO(CH₂)ₘ—**; —C(═O)O(CH₂)ₘNR¹¹C(═O)X₅C(═O)(CH₂)ₘNR¹¹C(═O)(CH₂)ₘ—**; —C(═O)O(CH₂)ₘX₆C(═O)X₁X₂C(═O)((CH₂)ₘO)ₙ(CH₂)ₘ—**; —(CH₂)ₘNR¹¹C(═O)X₁X₂C(═O)(CH₂)ₘ—**; —(CH₂)ₘ(CHOH)(CH₂)ₘNR¹¹C(═O)X₁X₂C(═O)(CH₂)ₘ**; —C(═O)X₆C(═O)(CH₂)ₘNR¹¹C(═O)X₁X₂C(═O)(CH₂)ₘ—**; —C(═O)X₄C(═O)NR(CH₂)ₘNR¹¹C(═O)(CH₂)ₘO(CH₂)ₘ—**; C(═O)(CH₂)ₘNR¹¹C(═O)X₁X₂C(═O)(CH₂)ₘ—**; —C(═O)O(CH₂)ₘX₆C(═O)X₁X₂C(═O)(CH₂)ₘ—**, or —C(═O)(CH₂)ₘNR¹¹C(═O)((CH₂)ₘO)ₙ(CH₂)ₘ—**, where the ** of L₁ indicates the point of attachment to R¹⁵.

In some embodiments, the compound is selected from:

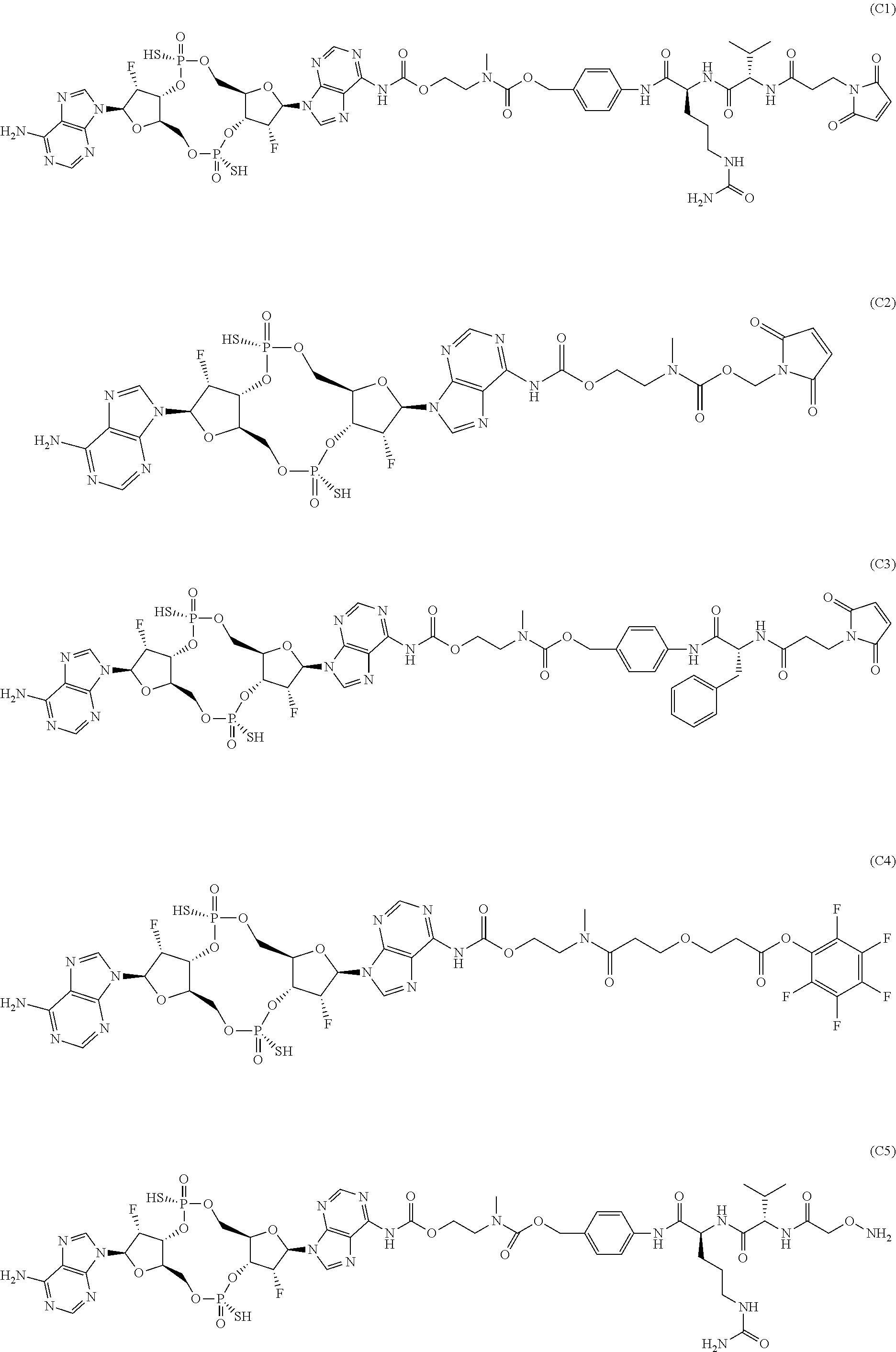

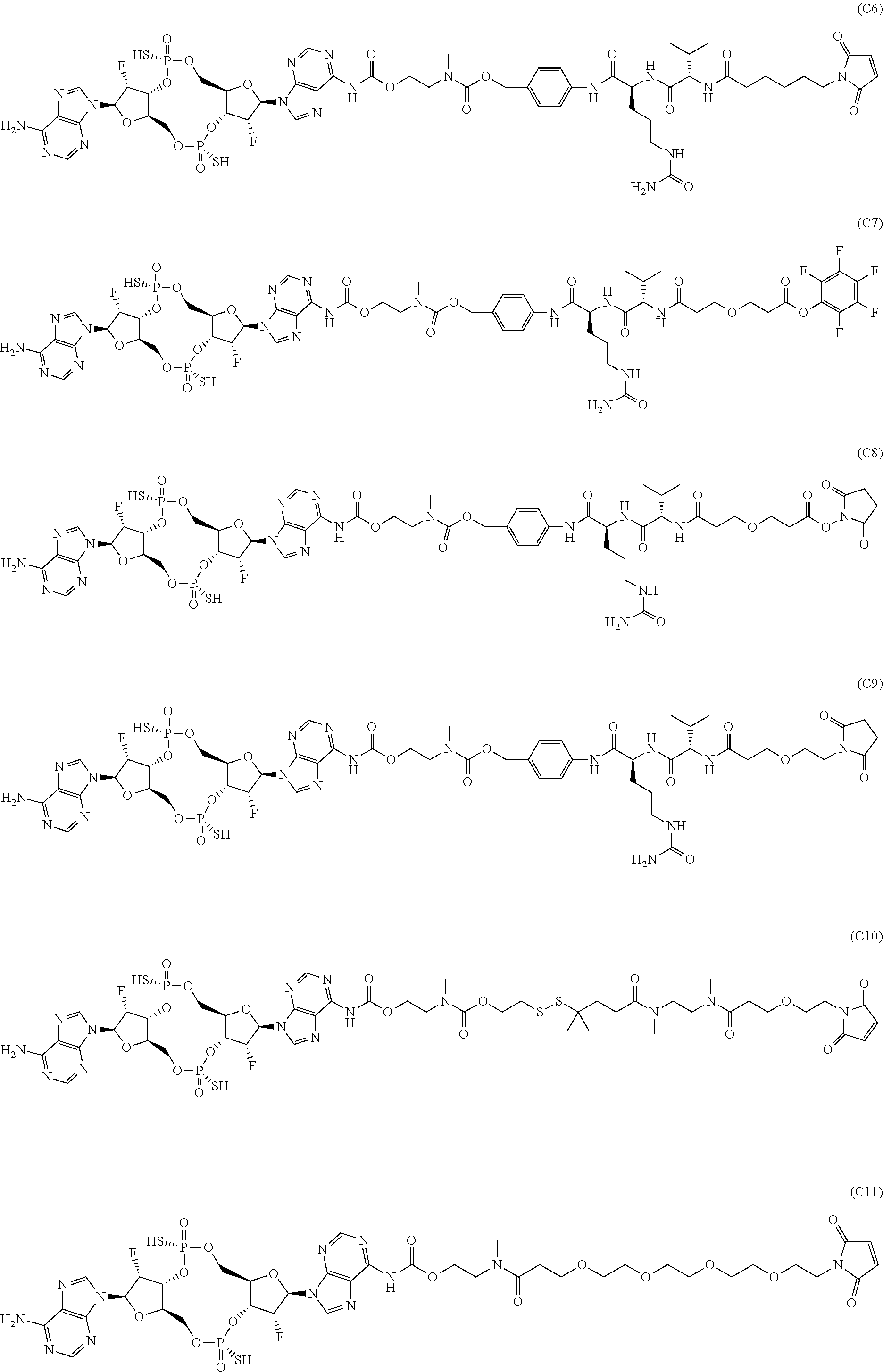

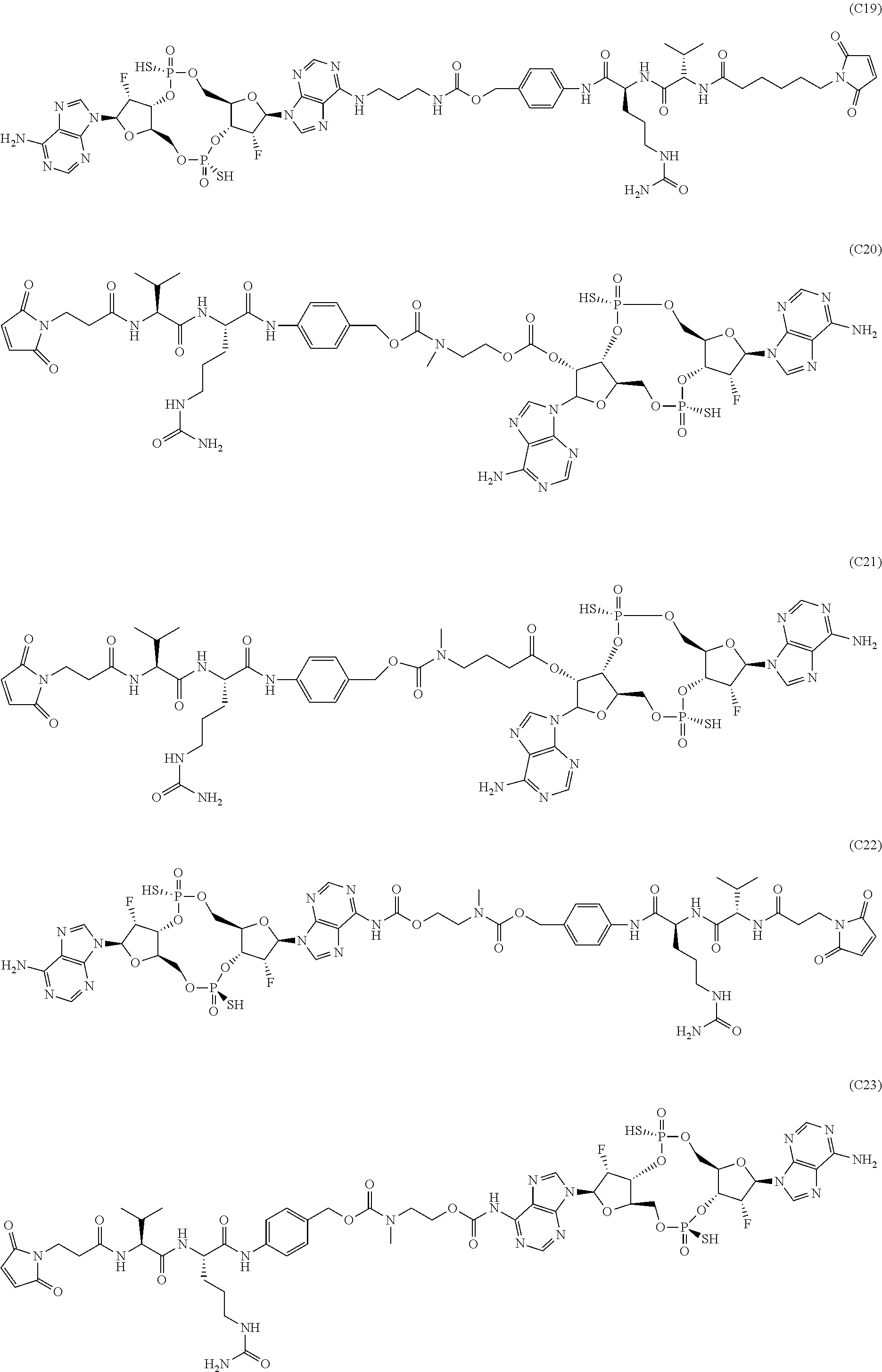

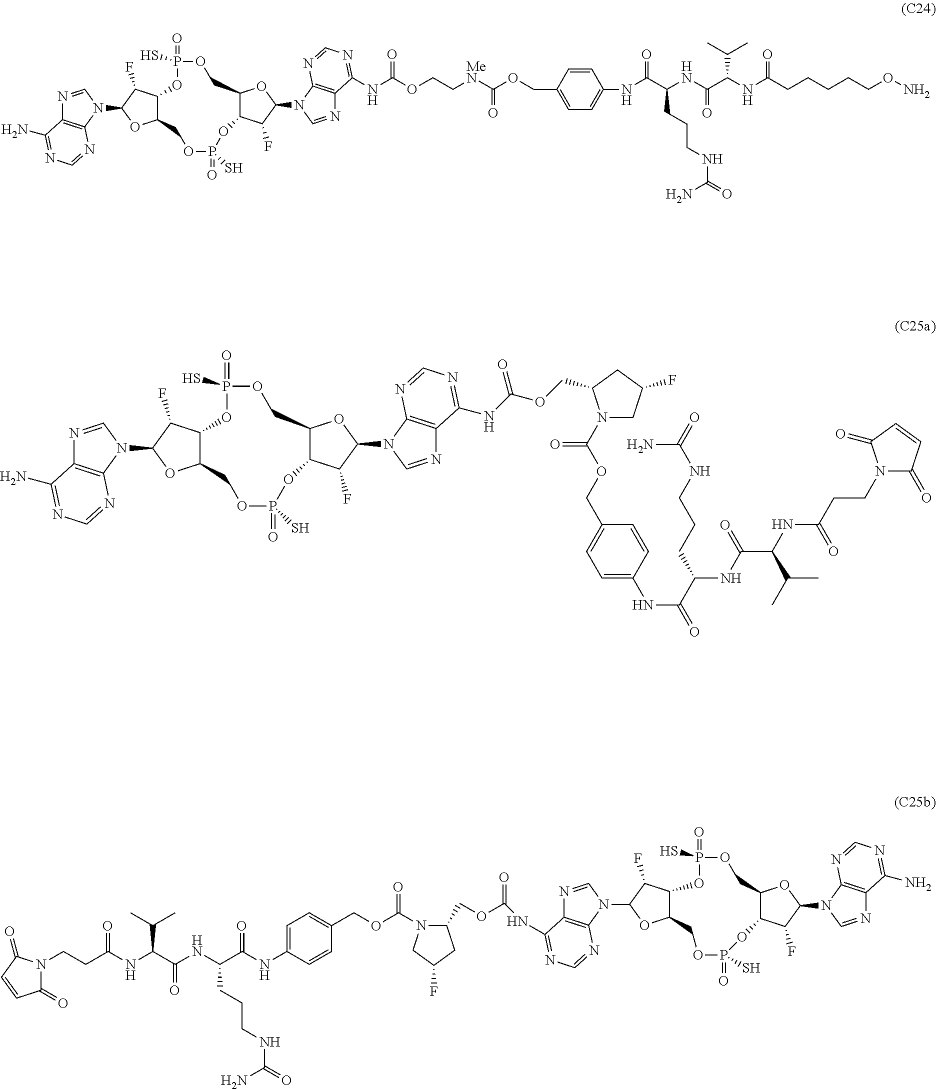

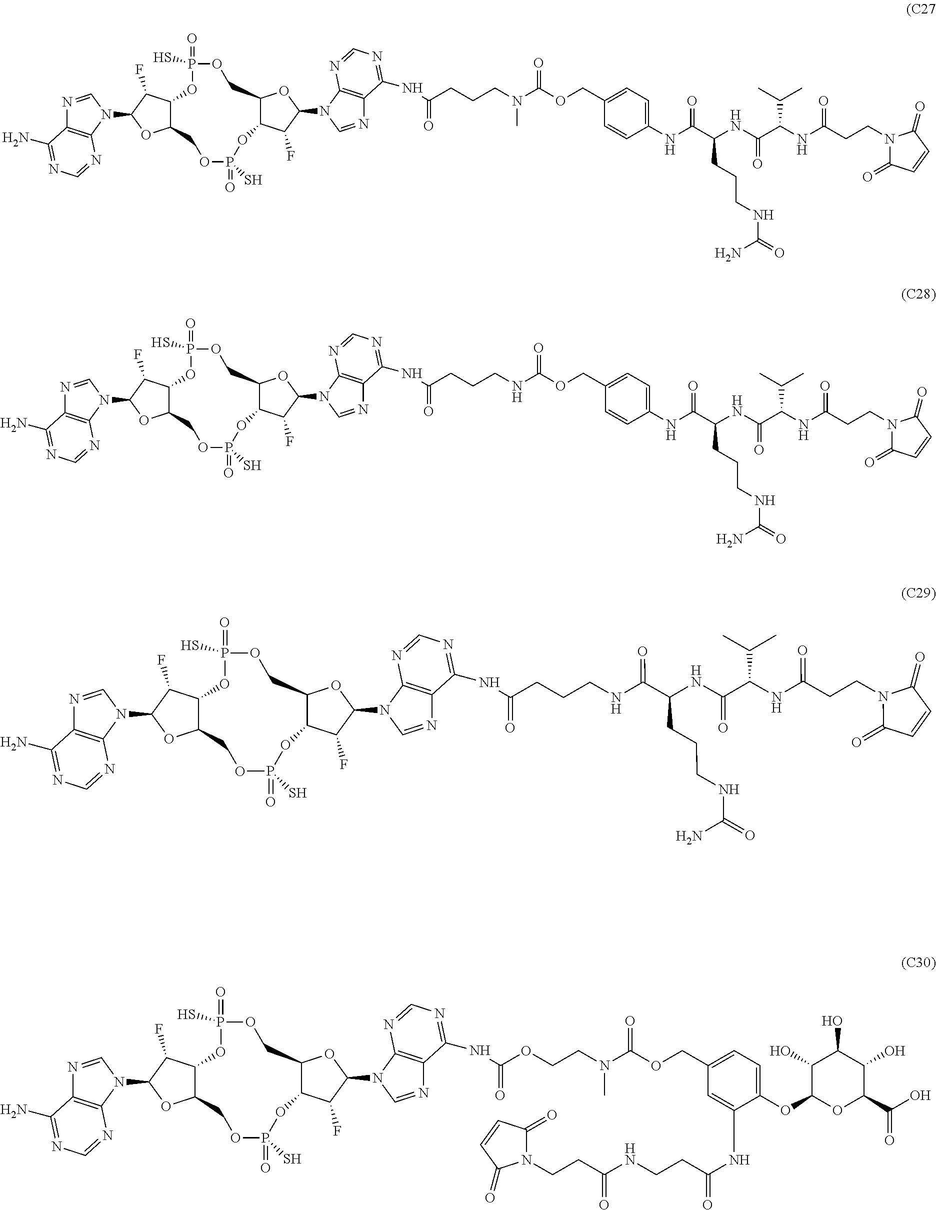

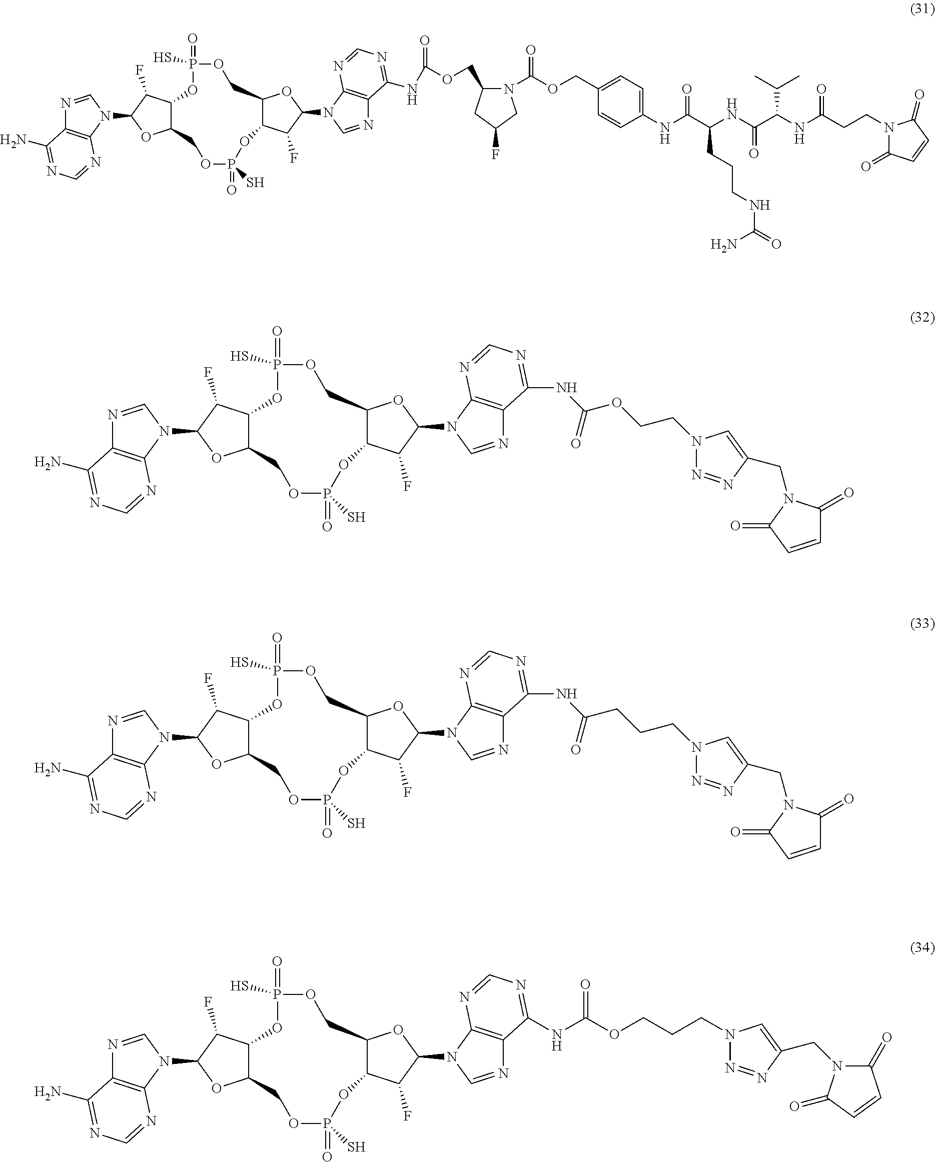

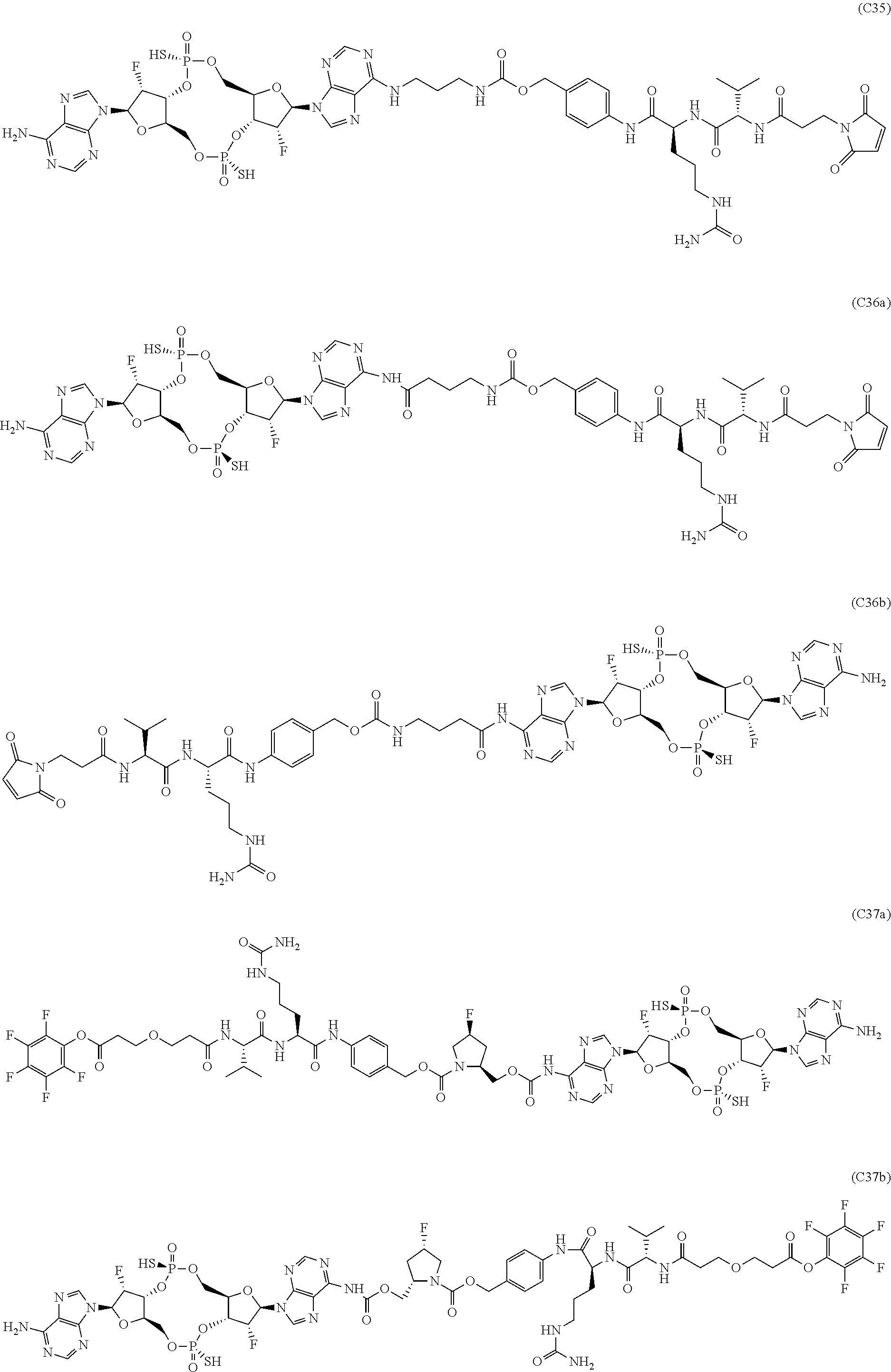

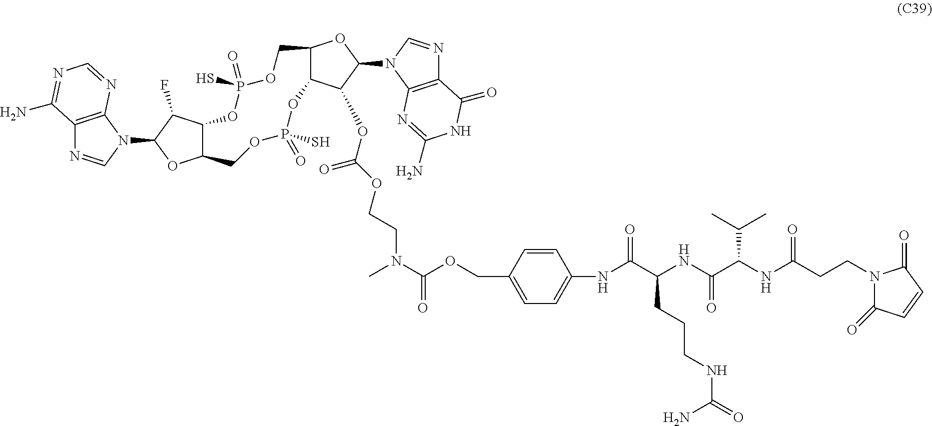 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(N)ncnc65)[C@H](F)[C@@H]4O[P@@](=O)(S)OC[C@H]3O[C@H]2n2cnc3c(=O)[nH]c(N)nc32)cc1]

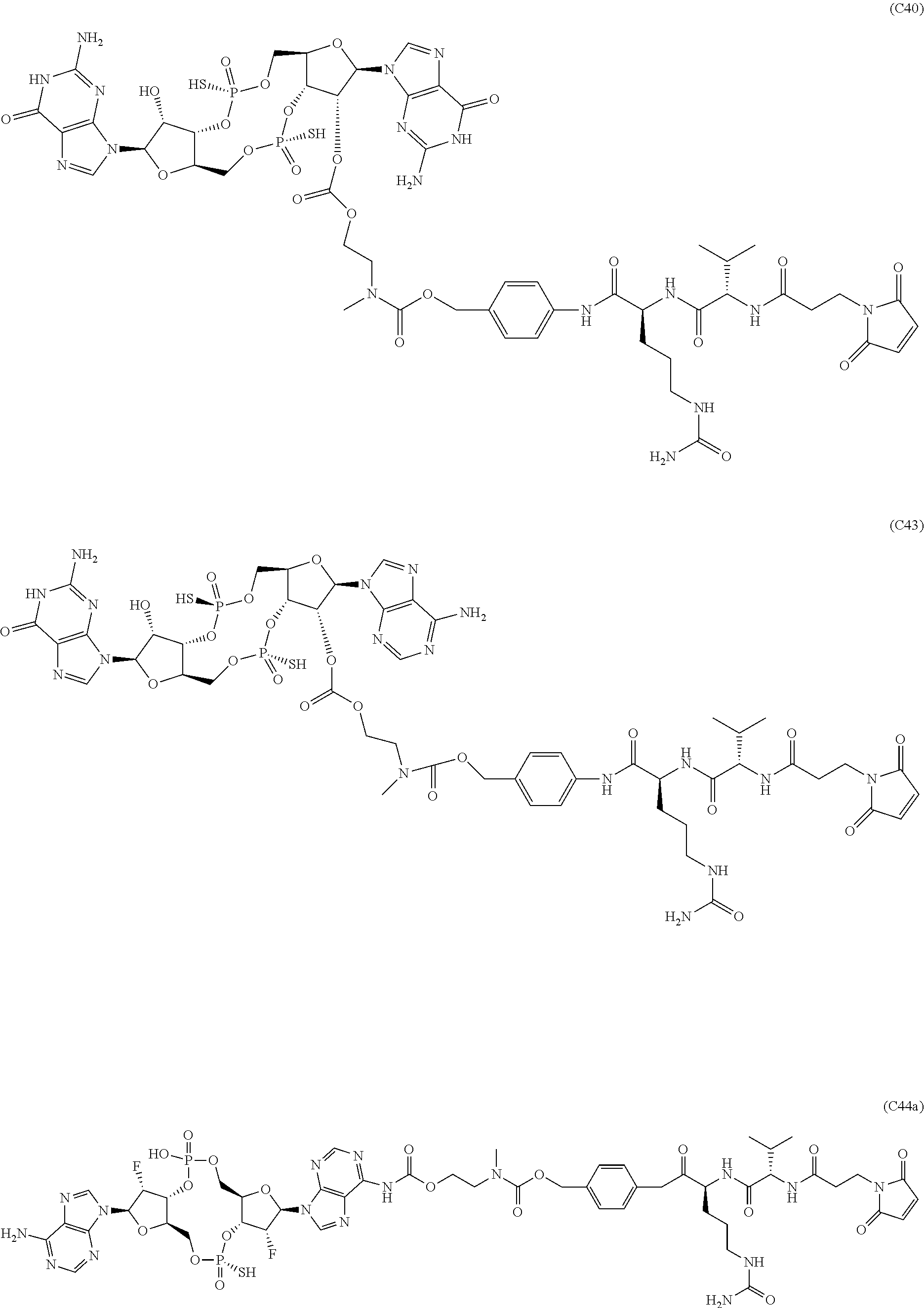

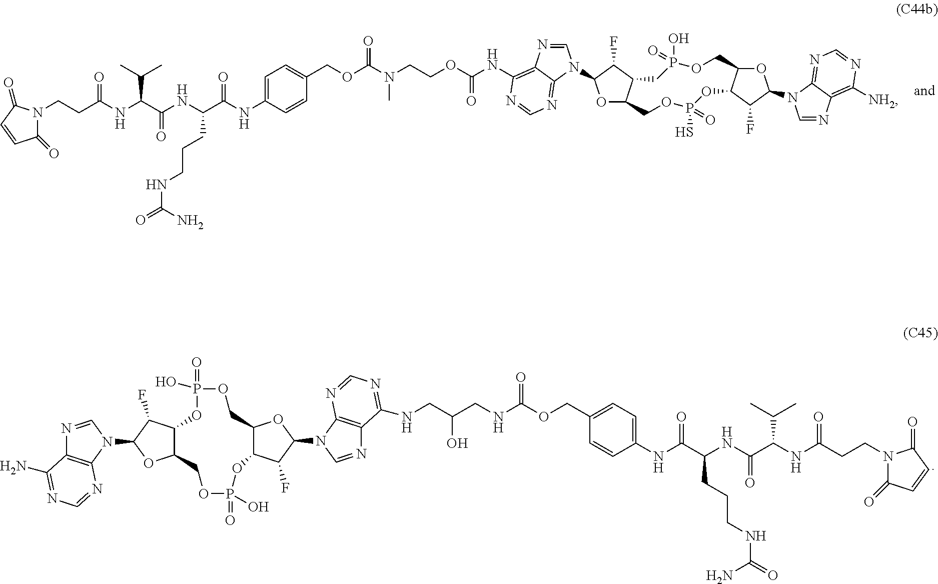 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4COP(=O)(O)C[C@H]3[C@H]2F)cc1 ; CC(C)[C@H](NC(=O)CCN1C(=O)C=CC1=O)C(=O)N[C@@H](CCCNC(N)=O)C(=O)Nc1ccc(COC(=O)NCC(O)CNc2ncnc3c2ncn3[C@@H]2O[C@@H]3COP(=O)(O)O[C@H]4[C@@H](F)[C@H](n5cnc6c(N)ncnc65)O[C@@H]4COP(=O)(O)O[C@H]3[C@H]2F)cc1]

In some embodiments, the compound is selected from:

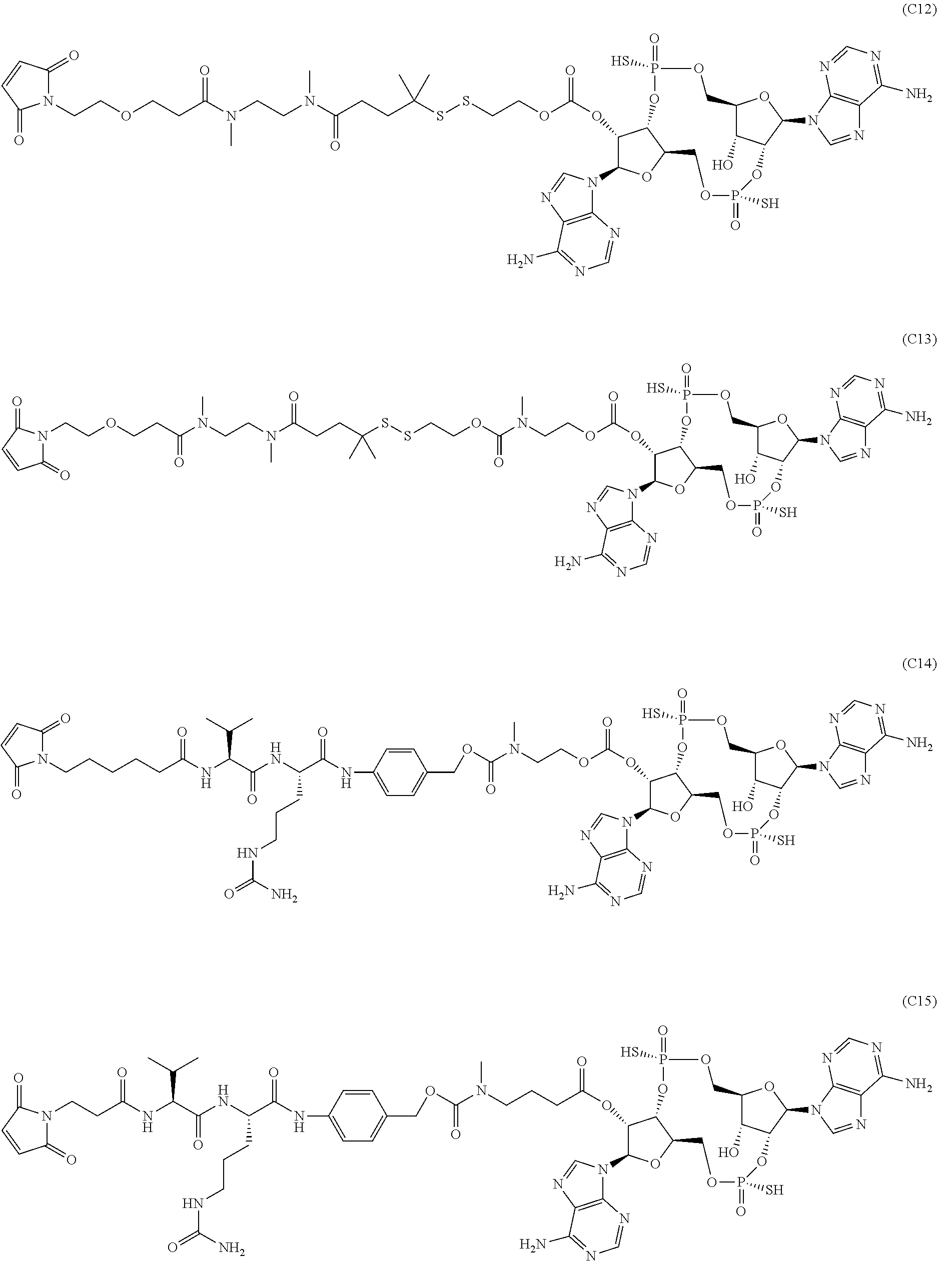

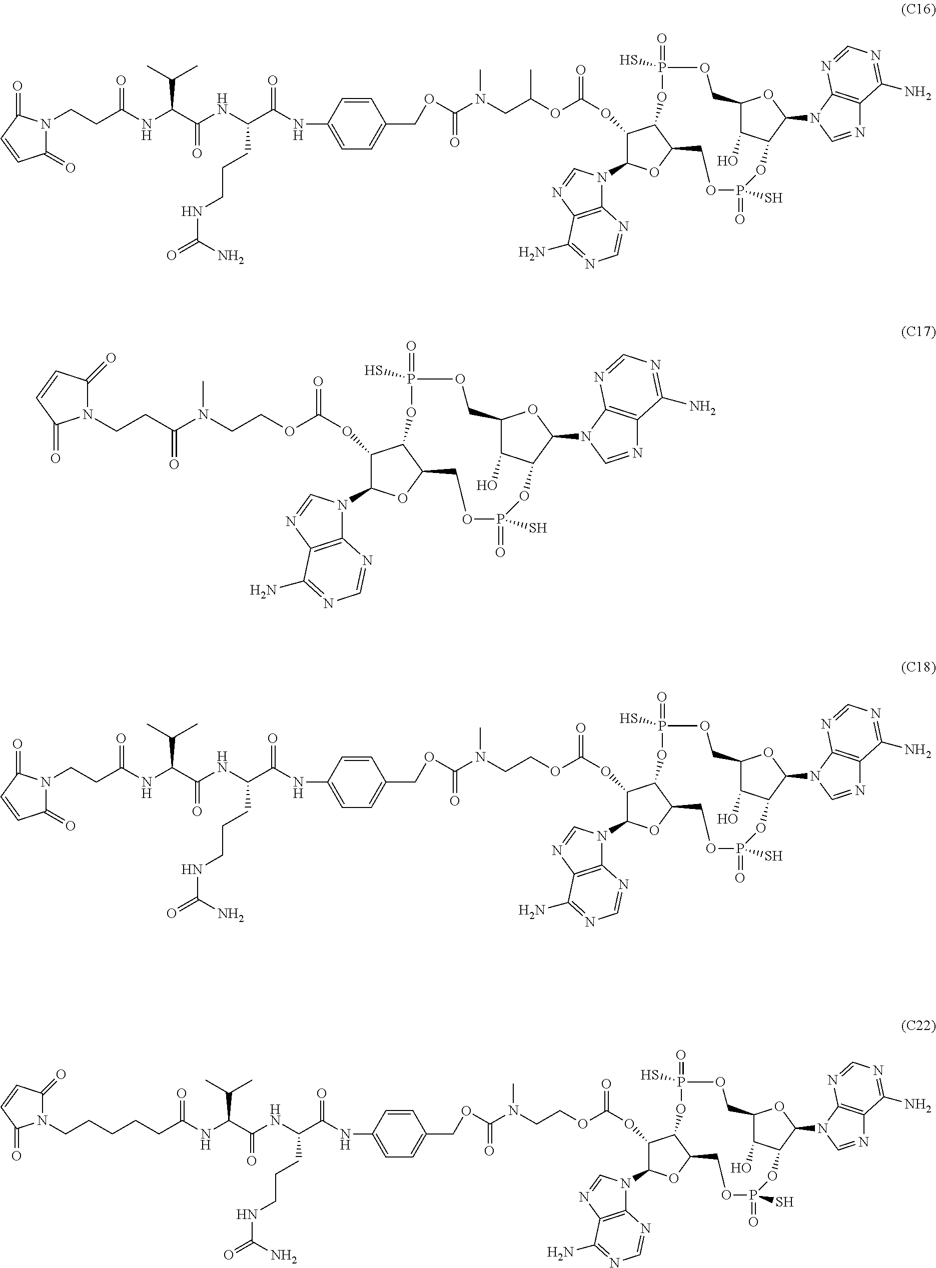

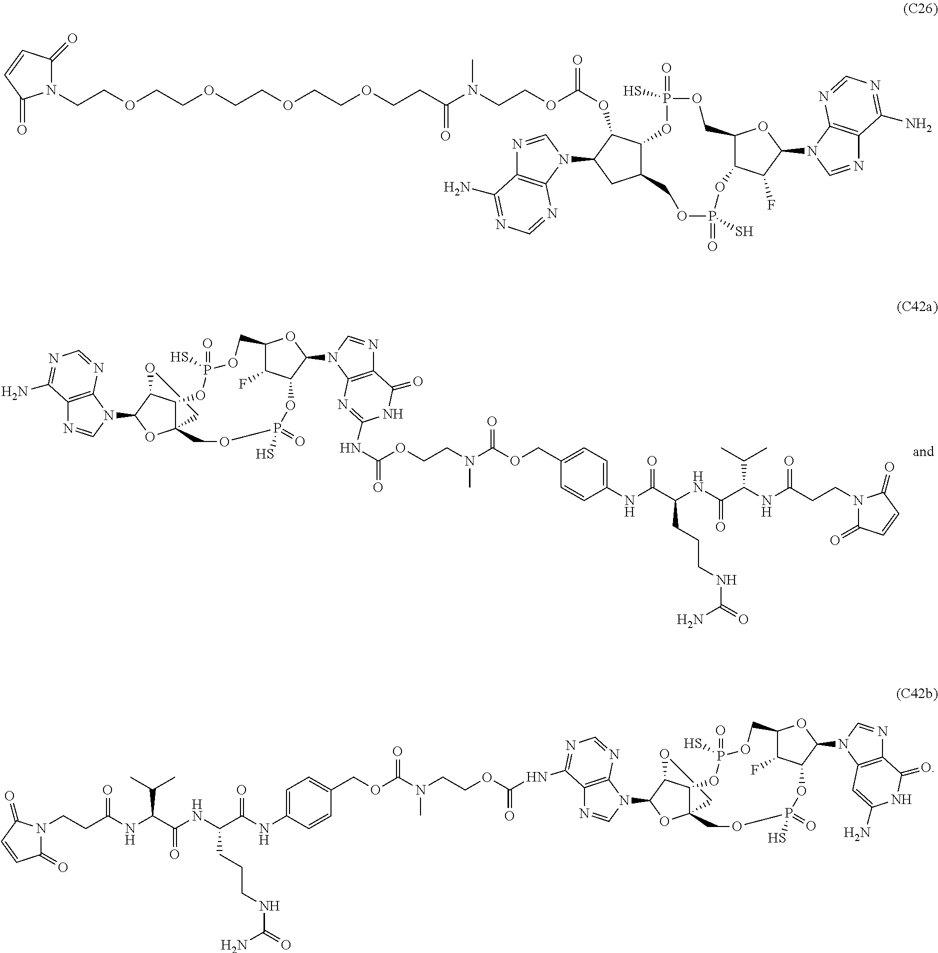

In some embodiments, the compound is selected from:

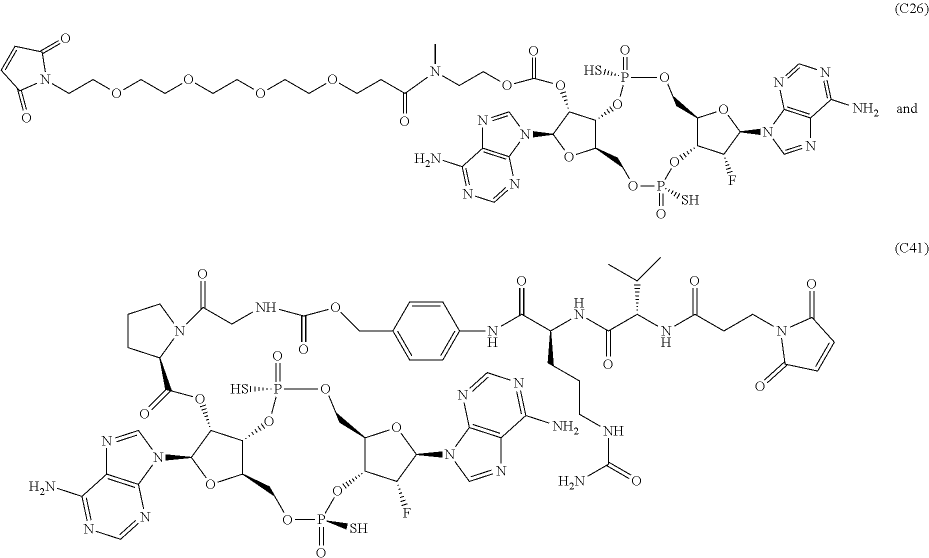

In some embodiments, the compound is selected from:

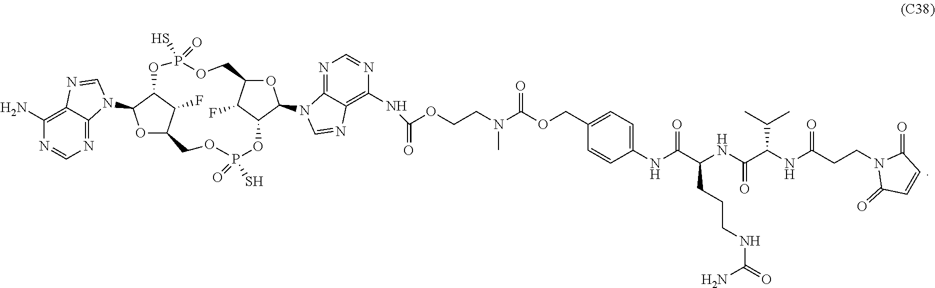 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@@H]4[C@H](F)[C@@H](CO[P@@](=O)(S)O[C@@H]2[C@@H]3F)O[C@H]4n2cnc3c(N)ncnc32)cc1]

### BRIEF DESCRIPTION OF THE DRAWINGS

FIGS. 1A-1D show exemplary data on DC-SIGN immunoconjugates activating human DCs and macrophages in vitro. All DC-SIGN antibody C1 Immunoconjugates induced downregulation of DC-SIGN on monocyte dendritic cells and macrophages, indicating target engagement (FIGS. 1A and 1C) and induced monocyte dendritic cell and macrophage activation as measured by CD86 upregulation (FIGS. 1B and 1D).

FIGS. 2A-2D show exemplary data on DC-SIGN immunoconjugates activating human DCs and macrophages in vitro. 2B2 (DAPA) immunoconjugates of C1, C18 and C31 induced downregulation of DC-SIGN on monocyte dendritic cells and macrophages (FIGS. 2A and 2C), indicating target engagement, and induced monocyte dendritic cell and macrophage activation as measured by CD86 upregulation (FIGS. 2B and 2D).

FIGS. 3A-3D show exemplary data on DAR2 DC-SIGN immunoconjugates activating human DCs and macrophages in vitro. Hz 2B2 (DAPA) C1 and Hz 2B2 (DAPA) DAR2 C1 induced downregulation of DC-SIGN on monocyte dendritic cells and macrophages (FIGS. 3A and 3C), indicating target engagement, and induced monocyte dendritic cell and macrophage activation as measured by CD86 upregulation (FIGS. 3B and 3D).

FIGS. 4A-4D show exemplary data on DC-SIGN immunoconjugates inducing cytokine production in Tg+ mice. All Hz 2B2 (DAPA) immunoconjugates except for C2 induced proinflammatory cytokine release at 6 hours post dose including IL-6 (FIG. 4C), TNFα (FIG. 4D) and IP-10 (FIG. 4B), and induced dendritic cell maturation as measured by CD86 upregulation at 24 hours post dose (FIG. 4A). * Indicates p value<0.05, ** indicated p value of <0.003, **** indicates a p value of <0.0001 compared to Tg− saline treated mice calculated using a one way ANOVA with Dunnett's test.

FIGS. 5A-5E show exemplary data on DC-SIGN immunoconjugates inducing cytokine production in Tg+ mice. Tg+ mice showed a robust increase in circulating plasma IP-10 (FIG. 5A), IFNβ (FIG. 5B), IL-6 (FIG. 5C), TNFα (FIG. 5D) and IL-12p70 (FIG. 5E). Plasma levels were analyzed by ELISA (IP-10 and IFNβ) or MesoScaleDiscovery Multiplex analysis (all other analytes). **** denotes p value of <0.0001 using an ANOVA with Tukey's test compared to Tg-2B2 hlgG1 DAPA C1 group.

FIGS. 6A-6E show exemplary data on DC-SIGN immunoconjugates inducing DC activation in a target dependent manner. DC-SIGN levels were significantly reduced in Tg+ mice treated with humanized 2B2 (DAPA)-C1 (FIG. 6A), indicating target engagement. Both CD80 and CD86 were highly upregulated in CD8+ and CD11 b+ DCs from mice treated with humanized 2B2 (DAPA)-C1 (FIGS. 6B-6E), demonstrating dendritic cell activation. ** denotes p value of <0.004, **** denotes p value of <0.0001 using an ANOVA with Tukey's test compared to Tg-2B2 hlgG1 DAPA C1 group.

FIGS. 7A-7D show exemplary data on DC-SIGN immunoconjugates activating DCs in Tg+ mice. Tg+ mice treated with anti-DC-SIGN (DAPA) C1 conjugates had a significant downregulation of surface DC-SIGN (FIGS. 7A and 7C), indicating target engagement. Tg+ mice treated with anti-DC-SIGN (DAPA) C1 conjugates also had a robust upregulation of CD86 on the surface of dendritic cells indicative of DC activation (FIGS. 7B and 7D). **** denotes a p value of <0.0001 compared to Tg+ mice treated with saline calculated using a one way ANOVA with Dunnett's test.

FIGS. 8A-8D show exemplary data on DC-SIGN immunoconjugates inducing cytokine production in Tg+ mice. Tg+ mice treated with anti-DC-SIGN (DAPA) C1 conjugates showed robust increases in plasma IP-10 (FIGS. 8A and 8C) and TNFα levels (FIGS. 8B and 8D) indicative of activation. * Denotes a p value of <0.05, ** denotes a p value of <0.002 **** denotes a p value of <0.0001 compared to Tg+ mice treated with saline calculated using a one way ANOVA with Dunnett's test.

FIGS. 9A-9B show exemplary data on DC-SIGN immunoconjugates with different Fc formats inducing cytokine production in Tg+ mice. DAPA and WT Fc formats as well as Fab2 and Fab C1 conjugates induced IP-10 production (FIG. 9A). DAPA, WT and Fab2 formats induced IL-12p70 production in Tg+ mice in a target dependent manner (FIG. 9B). **** denotes p value<0.0001, *** denotes p value of <0.001, * denotes p value of <0.05, using an ANOVA with Dunnett's test compared to Tg+ Isotype (DAPA) C1.

FIGS. 10A-10B show exemplary data on DC-SIGN immunoconjugates with different Fc formats inducing DC activation in Tg+ mice. DAPA and WT Fc formats as well as Fab2 and Fab versions of 2B2 C1 conjugates induced DC-SIGN downregulation (FIG. 10A), indicative of target engagement and CD86 upregulation on DCs (FIG. 10B), indicative of DC activation in Tg+ mice. **** denotes p value<0.0001 calculated using an ANOVA with Dunnett's test compared to Tg+ Isotype (DAPA) C1.

FIGS. 11A-11B show exemplary data on DC-SIGN immunoconjugates with a WT Fc format activating human DCs and macrophages in vitro. Both WT and DAPA 2B2 C1 conjugates induced downregulation of DC-SIGN on monocyte dendritic cells, indicating target engagement (FIG. 11A). Both WT and DAPA 2B2 C1 conjugates induce monocyte dendritic cell activation as measured by CD86 upregulation (FIG. 11B).

FIGS. 12A-12D show exemplary data on DC-SIGN immunoconjugates with different Fc formats inducing DC activation and cytokine production in Tg+ mice. Both DAPA and Fc silent versions of 2B2 C1 Immunoconjugates induced high levels of circulating IP-10 (FIG. 12A) and TNFα (FIG. 12B). Both DAPA and Fc silent versions of 2B2 C1 conjugates induced DC-SIGN downregulation (FIG. 12C) indicative of target engagement and CD86 upregulation on DCs (FIG. 12D) indicative of DC activation in Tg+ mice. ** denotes a p value of <0.01, *** denotes a p value of <0.001 compared to the appropriate Tg− control group calculated using an unpaired Student's t test. **** denotes p value of <0.0001 using a one way ANOVA with Dunnett's test compared to saline treated Tg+ mice.

FIGS. 13A-13C show exemplary data on DC-SIGN immunoconjugates inducing cytokine production in Tg+ mice in comparison to free CDN. Tg+ mice dosed with 1 mg/kg of 2B2 (DAPA) C1 or free T1-1 had increased circulating plasma IL-12p70 (FIG. 13C), TNFα (FIG. 13B) and IP-10 (FIG. 13A) levels compared to the untreated Tg+ mice and compared to mice treated with 10 μg of free T1-1 compound. ** denotes p value of 0.001, **** denotes p value of <0.0001 using an ANOVA with Tukey's test compared to Tg+ untreated, *** denotes p value of <0.0001 using unpaired Student's t test compared to Tg+ untreated.

FIGS. 14A-14C show exemplary data on DC-SIGN immunoconjugates inducing DC activation in comparison to free CDN. DC-SIGN levels were significantly reduced in Tg+ mice treated with humanized 2B2 (DAPA)-C1 (FIG. 14A), indicating target engagement. CD80 and CD86 were significantly upregulated on the surface of DCs from mice treated with 2B2 (DAPA) C1 and to a greater extent than was observed in animals treated with free T1-1 (FIGS. 14B and 14C). ** denotes p value of 0.001, *** denotes p value of 0.0006, **** denotes p value of <0.0001 using an ANOVA with Tukey's test compared to Tg+ saline.

FIGS. 15A-15D show exemplary data on 1G12 DC-SIGN immunoconjugates inducing DC activation and cytokine production. Tg+ mice treated with 1G12 (DAPA) C1 had a significant downregulation of surface DC-SIGN (FIG. 15A), indicating target engagement, and had a significant upregulation of CD86 on the surface of dendritic cells indicating activation (FIG. 15B). IP-10 (FIG. 15D) and IL-12p70 (FIG. 15C) plasma levels were significantly increased in Tg+ mice treated with 1G12 (DAPA) C1 at 6 hours post dose, indicative of on target activation through DC-SIGN. **** denotes p value of <0.0001 using a one way ANOVA with Dunnett's test compared to Tg− mice treated with 1G12.

FIGS. 16A-16C show exemplary data on DAR2 and DAR4 versions of DC-SIGN immunoconjugates inducing DC activation and cytokine production. Both antibody and payload matched doses of 2B2 (DAPA) DAR2 C1 induced DC activation as measured by CD86 upregulation (FIG. 16A) as well as IL-12p70 secretion (FIG. 16C) and IP-10 secretion (FIG. 16B) in a target dependent manner. **** denotes p value of <0.0001, *** denotes p value of ≤0.004, * denotes p value of 0.02 using an ANOVA with Tukey's test.

FIGS. 17A-17D show exemplary data on DC-SIGN immunoconjugates enhancing antibody responses to DNP-KLH and promoing isotype switching in Tg+ mice. Mice treated with 2B2 (DAPA) C1 show a significant increase in total DNP binding IgG (FIG. 17A) and also in IgG2a (FIG. 17C) and IgG3 (FIG. 17D) subclasses of DNP binding antibodies but not IgG1 (FIG. 17B). ** denotes p value of <0.01, * denotes p value of <0.05 in an unpaired Student's t test compared to mock treated group.

FIG. 18 shows exemplary data on DC-SIGN immunoconjugates delaying tumor growth in transgenic mice expressing DC-SIGN. DC-SIGN Tg+ mice treated with 1 mpk of 2B2 (DAPA) C1 conjugate had significantly delayed tumor growth kinetics, whereas Tg− mice did not show any impairment in tumor growth after dosing of 2B2 (DAPA) C1. Both Tg+ and Tg− mice treated with unconjugated 2B2 (DAPA) antibody did not show any change in tumor volume. **** denotes p value of <0.0001, * denotes p value of <0.05 in an unpaired Student's t test.

FIGS. 19A-19B show exemplary data on DC-SIGN immunoconjugates inducing upregulation of surface PDL1. Splenic CD11c high dendritic cells (FIG. 19A) and tumor resident dendritic cells and monocytic myeloid derived suppressor cells (mMDSCs) (FIG. 19B) showed a significant upregulation of surface PDL1 in Tg+ mice dosed with 1 mg/kg 2B2 (DAPA) C1. **** denotes p value of <0.0001, * denotes p value of 0.002 using an ANOVA with Tukey's test compared to Tg+2B2 (DAPA).

FIGS. 20A-20F show exemplary data on DC-SIGN immunoconjugates enhancing tumor T cell infiltration and T cell activation. Increased CD3+ T cells were observed 24 and 48 hours post dosing in Tg+ mice dosed with 2B2 (DAPA) C1 mice (FIGS. 20A and 20B). On day 7 post dose, a significant increase in CD8+ T cells (FIG. 20C) and a significant decrease in FoxP3+T regulatory cells (FIG. 20D) were observed in tumors from Tg+ mice dosed with 2B2 (DAPA) C1. Enhanced T cell activation as measured by CD69 upregulation was seen on CD4 and CD8 T cells in tumors from Tg+ mice dosed with 2B2 (DAPA) C1 24 hours post dose (FIGS. 20E and 20F). **** denotes p value of <0.0001, ** denotes p value of 50.003 using an ANOVA with Tukey's test compared to Tg+ Cysmab, ** denotes p value of 0.02 using Student's t test compared to Tg− 2B2 (DAPA) C1.

FIGS. 21A-21B show exemplary data on DC-SIGN immunoconjugates having enhanced anti-tumor activity in combination with anti-PDL1. Mice treated with the combination of 2B2 (DAPA) C1 and anti-PDL1 showed enhanced reduction in tumor volume (FIG. 21A) and enhanced infiltration of CD8 T cells in their tumors (FIG. 21B). **** p<0.0001, *** p<0.002, **p<0.01, *p<0.05 compared to isotype control (DAPA) C1 1 mg/kg using unpaired Student's t test.

FIGS. 22A-22B show exemplary data on DAR2 DC-SIGN immunoconjugates having enhanced anti-tumor activity in combination with anti-PDL1. Mice treated with the combination of humanized 2B2 (DAPA) C1 and anti-PDL1 or humanized 2B2 (DAPA) DAR2 C1 and anti-PDL1 showed a reduction in tumor volume compared to isotype control treated animals (FIG. 22A) and enhanced infiltration of CD8 T cells in their tumors compared to isotype control group (FIG. 22B). *** indicates p value of <0.001 using a one way ANOVA with Dunnet's test, ** indicates p value<0.01 calculated using an unpaired Student's t test, * indicates p value<0.05 calculated using an unpaired Student's t test.

FIGS. 23A-23B show exemplary data on DC-SIGN immunoconjugates with different payloads having enhanced anti-tumor activity in combination with anti-PDL1. Tg+ animals treated with 2B2 (DAPA) C31 in combination with anti PDL1 had significantly smaller tumors than Tg− animals (FIG. 23A). Tg+ animals treated with both 2B2 (DAPA) C31 and 2B2 (DAPA) C18 at 0.3 mg/kg in combination with anti PDL1 had significantly increased tumor CD8+ T cell infiltration compared to Tg− animals treated with the same regimen (FIG. 23B). p<0.01 using an unpaired Student's t test (compared to Tg− group with the same payload), ** p<0.01 using an ANOVA with Tukey's test (compared to Tg− group with the same payload).

FIGS. 24A-24B show exemplary data on 960K03 (DAPA)-C31 conjugate induces cytokine production in a target dependent manner. Transgenic mice expressing human DC-SIGN gene (Tg+) or transgene-negative littermate control (Tg−) mice were treated with 960K03 (DAPA) DAR4 C31 at 0.01, 0.03, 0.1, 0.3 or 1 milligram per kilogram body weight (mpk) intravenously (i.v.). Mice were bled 6 hours after dosing to collect plasma for analysis of circulating cytokine levels. Tg+ mice showed a robust increase in circulating plasma IP-10 (FIG. 24A) and TNFα (FIG. 24B) and Plasma levels were analyzed by ELISA (IP-10) or MesoScaleDiscovery Multiplex analysis (TNFα). **** denotes p value of <0.0001 and ** denotes a p value of <0.01 using a one way ANOVA with Sidak's test compared to the Tg− dose matched group.

FIGS. 25A-25B show exemplary data on 960K03 (DAPA)-C31 conjugate induces dendritic cell activation in a target dependent manner. Transgenic mice expressing human DC-SIGN gene (Tg+) or transgene-negative littermate control (Tg−) mice were treated with 960K03 (DAPA) DAR4 C31 at 0.01, 0.03, 0.1, 0.3 or 1 milligram per kilogram body weight (mpk) intravenously (i.v.). Spleens were harvested 24 hours post dose and analyzed by flow cytometry to look at CD11c+ dendritic cells. DC-SIGN levels were significantly reduced in Tg+ mice treated with 960K03 (DAPA) DAR4 C31 (FIG. 25A), indicating target engagement. CD86 was highly upregulated on CD11c+ dendritic cells in a dose dependent manner in Tg+ mice treatment with 960K03 (DAPA) DAR4 C31 (FIG. 25B), demonstrating dendritic cell activation. **** denotes p value of <0.0001 and ** denotes a p value of <0.01 using a one way ANOVA with Sidak's test compared to the Tg− dose matched group.

FIGS. 26A-26C show exemplary data on 960K03 (DAPA)-C31 conjugate is active in vitro on human monocyte DCs. Primary human monocytes were isolated from a leukapheresis using magnetic bead selection and frozen for storage in liquid nitrogen. For monocyte DC (moDC) differentiation, cells were thawed and incubated in media containing GM-CSF and IL-4 for 7 days. After the differentiation process for both moDC and moMacs, media was washed off and replaced with fresh media containing isotype control (DAPA) or 960K03 (DAPA) conjugated to C31 payload. Free T1-1 compound was used as a control. 24 hours after incubation with indicated compounds, cells were evaluated by flow cytometry for activation. 960K03 (DAPA) C31 conjugate induced downregulation of DC-SIGN on monocyte dendritic cells, indicating target engagement (FIG. 26A). 960K03 (DAPA) C31 induced monocyte dendritic cell activation (as measured by CD86 upregulation) with less payload than the isotype control (DAPA) C31 conjugate or unconjugated T1-1 (FIG. 26B). 960K03 (DAPA) C31 also induced IP-10 secretion into the culture supernatant at a higher concentration with less payload than the isotype control (DAPA) C31 conjugate or unconjugated T1-1 (FIG. 26C).

FIGS. 27A-27B show exemplary data on 960K03 (DAPA)-C31 conjugate has anti-tumor activity in combination with anti-PDL1 therapy. Female transgenic mice expressing human DC-SIGN gene (Tg+) or DC-SIGN negative littermate controls (Tg−) were implanted with 2.5×10⁵ MC38 tumor cells subcutaneously in the hind flank. Tumors were measured 3 times weekly throughout the course of the study. When tumors reached 100-200 cubic millimeters (mm³), mice were given a single treatment of 0.1, 0.3 or 1 mg/kg 960K03 (DAPA) DAR4 C31. A control group received no 960K03 (DAPA) DAR4 C31. All groups were given 2 doses of anti-PDL1 clone 10F.9G2 at 10 mg/kg throughout the course of the study (every 3-4 days). Mice treated with the combination of 960K03 (DAPA) DAR4 C31 and anti-PDL1 showed enhanced reduction in tumor volume at both 0.3 mg/kg as well as the 1 mg/kg dose levels of 960K03 (DAPA) DAR4 C31 (FIG. 27A). **p<0.01, *p<0.05 compared to dose matched Tg− control group using unpaired Student's t test. 7 days after dosing with 960K03 (DAPA) DAR4 C31, tumors were analyzed by flow cytometry for T cell infiltration. Mice treated with the 960K03 (DAPA) DAR4 C31 and anti-PDL1 showed enhanced infiltration of CD8+ T cells in their tumors when compared to dose matched Tg− controls (FIG. 27B). **p<0.01 compared to dose matched Tg− control group using a one-way ANOVA with Tukey's test.

### DETAILED DESCRIPTION OF THE INVENTION

Various enumerated embodiments of the invention are described herein. It will be recognized that features specified in each embodiment may be combined with other specified features to provide further embodiments of the present invention.

Throughout the text of this application, should there be a discrepancy between the text of the specification (e.g., Table 8) and the sequence listing, the text of the specification shall prevail.

### Definitions

The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl, ethyl, 1-methylethyl, n-propyl, isopropyl, n-butyl, isobutyl, sec-butyl, tert-butyl, n-pentyl, isopentyl and hexyl.

The term “C₂-C₆alkenyl”, as used herein, refers to a straight or branched hydrocarbon chain radical group consisting solely of carbon and hydrogen atoms, containing at least one double bond, having from two to six carbon atoms, which is attached to the rest of the molecule by a single bond. Non-limiting examples of “C₂-C₆alkenyl” groups include ethenyl, prop-1-enyl, but-1-enyl, pent-1-enyl, pent-4-enyl and penta-1,4-dienyl.

The term “C₂-C₆alkynyl”, as used herein, refers to a straight or branched hydrocarbon chain radical group consisting solely of carbon and hydrogen atoms, containing at least one triple bond, having from two to six carbon atoms, and which is attached to the rest of the molecule by a single bond. Non-limiting examples of “C₂-C₆alkynyl” groups include ethynyl, prop-1-ynyl, but-1-ynyl, pent-1-ynyl, pent-4-ynyl and penta-1,4-diynyl.

The term “C₁-C₆alkylene”, as used herein, refers to a bivalent straight or branched hydrocarbon chain radical consisting solely of carbon and hydrogen atoms, containing no unsaturation, having from one to six carbon atoms.

The term “C₂-C₆alkenyl”, as used herein, refers to a bivalent straight or branched hydrocarbon chain radical group consisting solely of carbon and hydrogen atoms, containing at least one double bond, having from two to six carbon atoms.

The term “C₂-C₆alkynyl”, as used herein, refers to a bivalent straight or branched hydrocarbon chain radical group consisting solely of carbon and hydrogen atoms, containing at least one triple bond, having from two to six carbon atoms.

The term “C₁₋₆alkoxyalkyl”, as used herein, refers to a radical of the formula —Ra—O—Ra, where each Ra is independently a C₁₋₆alkyl radical as defined above. The oxygen atom may be bonded to any carbon atom in either alkyl radical. Examples of C₁₋₆alkoxy include, but are not limited to, methoxy-methyl, methoxy-ethyl, ethoxy-ethyl, 1-ethoxy-propyl and 2-methoxy-butyl.

The term “C₁-C₆hydroxyalkyl”, as used herein, refers to a C₁₋₆alkyl radical as defined above, wherein one of the hydrogen atoms of the C₁₋₆alkyl radical is replaced by OH. Examples of hydroxyC₁₋₆alkyl include, but are not limited to, hydroxy-methyl, 2-hydroxy-ethyl, 2-hydroxy-propyl, 3-hydroxy-propyl and 5-hydroxy-pentyl

The term “C₃-C₈cycloalkyl,” as used herein, refers to a saturated, monocyclic, fused bicyclic, fused tricyclic or bridged polycyclic ring system. Non-limiting examples of fused bicyclic or bridged polycyclic ring systems include bicyclo[1.1.1]pentane, bicyclo[2.1.1]hexane, bicyclo[2.2.1]heptane, bicyclo[3.1.1]heptane, bicyclo[3.2.1]octane, bicyclo[2.2.2]octane and adamantanyl. Non-limiting examples monocyclic C₃-C₈cycloalkyl groups include cyclopropyl, cyclobutyl, cyclopentyl and cyclohexyl groups.

The term “C₁-C₆haloalkyl”, as used herein, refer to the respective “C₁-C₆alkyl”, as defined herein, wherein at least one of the hydrogen atoms of the “C₁-C₆alkyl” is replaced by a halo atom. The C₁-C₆haloalkyl groups can be monoC₁-C₆haloalkyl, wherein such C₁-C₆haloalkyl groups have one iodo, one bromo, one chloro or one fluoro. Additionally, the C₁-C₆haloalkyl groups can be diC₁-C₆haloalkyl wherein such C₁-C₆haloalkyl groups can have two halo atoms independently selected from iodo, bromo, chloro or fluoro. Furthermore, the C₁-C₆haloalkyl groups can be polyC₁-C₆haloalkyl wherein such C₁-C₆haloalkyl groups can have two or more of the same halo atoms or a combination of two or more different halo atoms. Such polyC₁-C₆haloalkyl can be perhaloC₁-C₆haloalkyl where all the hydrogen atoms of the respective C₁-C₆alkyl have been replaced with halo atoms and the halo atoms can be the same or a combination of different halo atoms. Non-limiting examples of C₁-C₆haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, trifluoroethyl, difluoropropyl, dichloroethyl and dichloropropyl.

The term “C₂-C₆haloalkenyl”, as used herein, refer to the respective “C₁-C₆alkenyl”, as defined herein, wherein at least one of the hydrogen atoms of the “C₁-C₆alkenyl” is replaced by a halo atom. The C₂-C₆haloalkenyl groups can be monoC₁-C₆haloalkenyl, wherein such C₁-C₆haloalkenyl groups have one iodo, one bromo, one chloro or one fluoro. Additionally, the C₂-C₆haloalkenyl groups can be diC₂-C₆haloalkenyl wherein such C₂-C₆haloalkenyl groups can have two halo atoms independently selected from iodo, bromo, chloro or fluoro. Furthermore, the C₂-C₆haloalkenyl groups can be polyC₂-C₆haloalkenyl wherein such C₂-C₆haloalkenyl groups can have two or more of the same halo atoms or a combination of two or more different halo atoms.

The term “C₂-C₆haloalkynyl”, as used herein, refer to the respective “C₁-C₆alkynyl”, as defined herein, wherein at least one of the hydrogen atoms of the “C₁-C₆alkynyl” is replaced by a halo atom. The C₂-C₆haloalkynyl groups can be monoC₁-C₆haloalkynyl, wherein such C₁-C₆haloalkynyl groups have one iodo, one bromo, one chloro or one fluoro. Additionally, the C₂-C₆haloalkynyl groups can be diC₂-C₆haloalkynyl wherein such C₂-C₆haloalkynyl groups can have two halo atoms independently selected from iodo, bromo, chloro or fluoro. Furthermore, the C₂-C₆haloalkynyl groups can be polyC₂-C₆haloalkynyl wherein such C₂-C₆haloalkenyl groups can have two or more of the same halo atoms or a combination of two or more different halo atoms.

The term “heteroalkyl”, as used herein, refers to an “alkyl” moiety wherein at least one of the carbon atoms has been replaced with a heteroatom such as O S, or N.

The term “3 to 6 membered heterocycloalkyl,” as used herein refers to a monocyclic ring structure having 3 to 6 ring members, wherein one to two of the ring members are independently selected from N, NH, NR¹⁶, O or —S—, wherein R¹⁶ is C₁-C₆alkyl. Non-limiting examples of 3-6 membered heterocycloalkyl groups, as used herein, include aziridin-1-yl, aziridin-2-yl, aziridin-3-yl, azetadinyl, azetadin-1-yl, azetadin-2-yl, azetadin-3-yl, oxetanyl, oxetan-2-yl, oxetan-3-yl, oxetan-4-yl, thietanyl, thietan-2-yl, thietan-3-yl, thietan-4-yl, pyrrolidinyl, pyrrolidin-1-yl, pyrrolidin-2-yl, pyrrolidin-3-yl, pyrrolidin-4-yl, pyrrolidin-5-yl, tetrahydrofuranyl, tetrahydrofuran-2-yl, tetrahydrofuran-3-yl, tetrahydrofuran-4-yl, tetrahydrofuran-5-yl, tetrahydrothienyl, tetrahydrothien-2-yl, tetrahydrothien-3-yl, tetrahydrothien-4-yl, tetrahydrothien-5-yl, piperidinyl, piperidin-1-yl, piperidin-2-yl, piperidin-3-yl, piperidin-4-yl, piperidin-5-yl, piperidin-6-yl, tetrahydropyranyl, tetrahydropyran-2-yl, tetrahydropyran-3-yl, tetrahydropyran-4-yl, tetrahydropyran-5-yl, tetrahydropyran-6-yl, tetrahydrothiopyranyl, tetrahydrothiopyran-2-yl, tetrahydrothiopyran-3-yl, tetrahydrothiopyran-4-yl, tetrahydrothiopyran-5-yl, tetrahydrothiopyran-6-yl, piperazinyl, piperazin-1-yl, piperazin-2-yl, piperazin-3-yl, piperazin-4-yl, piperazin-5-yl, piperazin-6-yl, morpholinyl, morpholin-2-yl, morpholin-3-yl, morpholin-4-yl, morpholin-5-yl, morpholin-6-yl, thiomorpholinyl, thiomorpholin-2-yl, thiomorpholin-3-yl, thiomorpholin-4-yl, thiomorpholin-5-yl, thiomorpholin-6-yl, oxathianyl, oxathian-2-yl, oxathian-3-yl, oxathian-5-yl, oxathian-6-yl, dithianyl, dithian-2-yl, dithian-3-yl, dithian-5-yl, dithian-6-yl, dioxolanyl, dioxolan-2-yl, dioxolan-4-yl, dioxolan-5-yl, thioxanyl, thioxan-2-yl, thioxan-3-yl, thioxan-4-yl, thioxan-5-yl, dithiolanyl, dithiolan-2-yl, dithiolan-4-yl, dithiolan-5-yl, pyrazolidinyl, pyrazolidin-1-yl, pyrazolidin-2-yl, pyrazolidin-3-yl, pyrazolidin-4-yl and pyrazolidin-5-yl.

The term “heterocyclyl”, as used herein, includes partially saturated or aromatic monocyclic or fused bicyclic heterocyclyl containing from 5-10 ring members selected from carbon atoms and 1 to 5 heteroatoms, and each heteroatoms is independently selected from O, N or S. In a preferred embodiment, the heteroatoms are nitrogen. Non-limiting examples of substituents include oxo, halo, C₁₋₆alkyl, C₁₋₆alkoxy, amino, C₁₋₆alkylamino, di-C₁₋₆alkylamino. The heterocyclic group can be attached at a heteroatom or a carbon atom.

For fused bicyclic heterocyclyl system, the system can be fully aromatic (i.e. both rings are aromatic). When fully aromatic, the heterocyclyl can be referred to as heteroaryl. Examples of aromatic bicyclic heteroaryl include 9-10 membered fused bicyclic heteroaryl having 2-5 heteroatoms, preferably nitrogen atoms. Non-limiting examples are: pyrrolo[2,3-b]pyridinyl, pyrrolo[3,2-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-d]pyridinyl, pyrazolo[3,4-b]pyridinyl, imidazo[1,2-a]pyridinyl, pyrazolo[1,5-a]pyridinyl, pyrrolo[1,2-b]pyridazinyl, imidazo[1,2-c]pyrimidinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, or pyrimido[4,5-d]pyrimidinyl. Other non-limiting examples of fused bicyclic heterocyclyls include

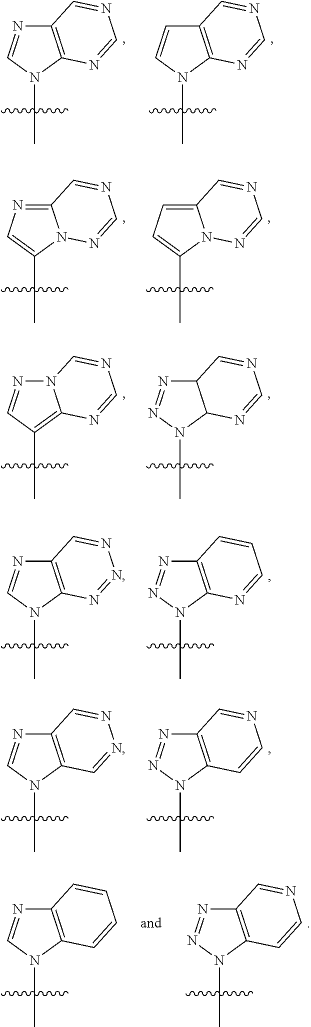

Additionally, bicyclic heterocyclyl ring systems include heterocyclyl ring systems wherein one of the fused rings is aromatic but the other is non-aromatic. For such systems, the heterocyclyl is said to be partially saturated. Examples of partially saturated bicyclic system are for example dihydropurinones such as 2-amino-1,9-dihydro-6H-purin-9-yl-6-one and 1,9-dihydro-6H-purin-9-yl-6-one. Other examples of partially saturated bicyclic system are

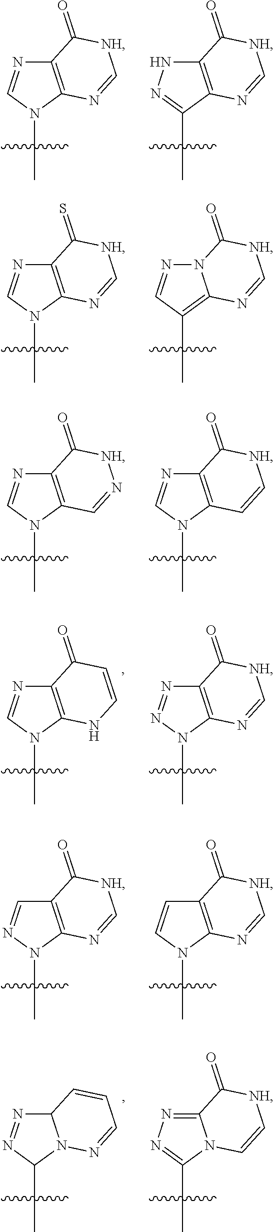

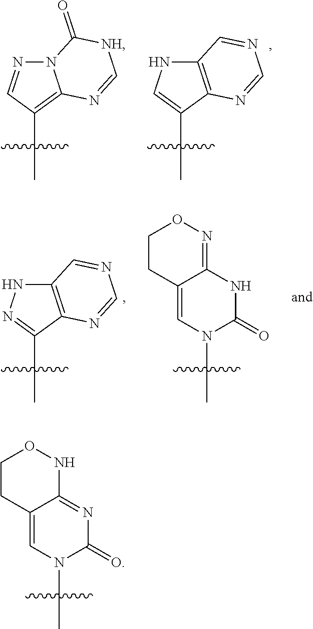

Heterocyclyl also includes a 5- or 6-membered ring aromatic heterocyclyl having 2 to 3 heteroatom (preferably nitrogen) (also referred to as 5- to 6-membered heteroaryl). Examples of monocyclic heteroaryl are: imidazolyl, pyrazolyl, thiazolyl, isothiazolyl, 1, 2, 3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, isothiazol-3-yl, isothiazol-4-yl, isothiazol-5-yl, oxazol-2-yl, oxazol-4-yl, oxazol-5-yl, isoxazol-3-yl, isoxazol-4-yl, isoxazol-5-yl, 1,2,4-triazol-3-yl, 1,2,4-triazol-5-yl, 1,2,3-triazol-4-yl, 1,2,3-triazol-5-yl, tetrazolyl, pyrid-2-yl, pyrid-3-yl, or pyridyl-4-yl, pyridazin-3-yl, pyridazin-4-yl, pyrazin-3-yl, 2-pyrazin-2-yl, pyrazin-4-yl, pyrazin-5-yl, 2-, 4-, or 5-pyrimidin-2-yl, pyrimidin-4-yl, pyrimidin-5-yl.

Heterocyclyl also includes 6-membered monocyclic partially saturated ring having 1-3 heteroatoms (preferably nitrogen). Examples of partially saturated monocyclic heterocyclyl are pyrimidine-one and pyrimidine-dione, specifically pyrimidin-2(1H)-one and pyrimidin-1-yl-2,4(1H, 3H)-dione.

Heterocyclyl can exist in various tautomeric forms. For example, when a heterocyclyl moiety is substituted with an oxo group next to a nitrogen atom, the invention also pertains to its hydroxy tautomeric form. For example, 2-amino-1,9-dihydro-6H-purin-6-one can tautomerize into 2-amino-9H-purin-6-ol. The tautomerization is represented as follow:

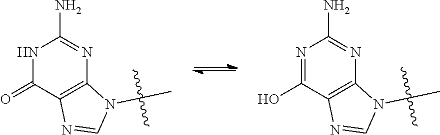 [SMILES: *n1cnc2c(=O)[nH]c(N)nc21 ; *n1cnc2c(O)nc(N)nc21]

As used herein, the term tautomer is used to designate 2 molecules with the same molecular formula but different connectivity, which can interconvert in a rapid equilibrium. Additional examples of tautomers are phosporothioic acid which can exist in an equilibrium as shown below.

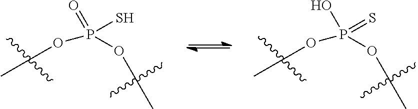 [SMILES: *OP(=O)(S)O* ; *OP(O)(=S)O*]

Similarly, phosphoric acid exists as 2 tautomeric forms which interconvert in an equilibrium.

Additional examples of tautomers are phosporothioic acid which can exist in an equilibrium as shown below.

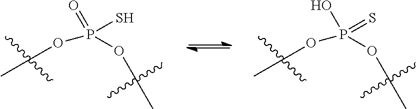 [SMILES: *OP(=O)(S)O* ; *OP(O)(=S)O*]

Similarly, phosphoric acid exists as 2 tautomeric forms which interconvert in an equilibrium.

In addition the phosporothioic acid and phosphoric acid moieties can exist in the respective equilibrium as shown below.

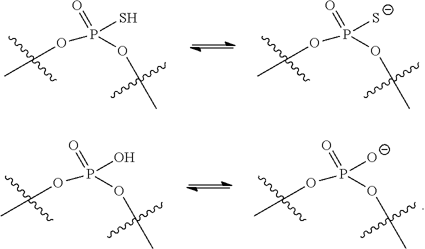 [SMILES: *OP(=O)(S)O* ; *OP(=O)([S-])O* ; *OP(=O)(O)O* ; *OP(=O)([O-])O*]

The term “Drug moiety”, as used herein, refers to a compound which binds to Stimulator of Interferon Genes (STING) receptor and which comprises one or more functional groups each of which is capable of forming a covalent bond with a linker. Examples of such functional groups include, but are not limited to, primary amines, secondary amines, hydroxyls, thiols, alkenes, alkynes and azides. In certain embodiments, such functional groups include reactive groups of Table 5 provided herein.

The term “sugar moiety”, as used herein, refers to the following ring structures of the compounds of the invention

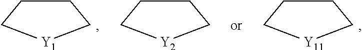

wherein Y₁, Y₂ and Y₃ are each independently selected from —O—, —S—, —S(═O)—, —SO₂—, —CH₂—, or —CF₂—.

As used herein, when partial structures of the compounds are illustrated a wavy line ( [symbol] ) indicates the point of attachment of the partial structure to the rest of the molecule.

As used herein, “DC-SIGN” (Dendritic Cell-Specific Intercellular adhesion molecule-3-Grabbing Non-integrin, also known as CD209; CD209 molecule, CDSIGN; CLEC4L; DC-SIGN1) refers to a transmembrane receptor and is referred to as DC-SIGN because of its expression on the surface of dendritic cells and macrophages. The protein is involved in the innate immune system and recognizes numerous evolutionarily divergent pathogens ranging from parasites to viruses with a large impact on public health. The protein is organized into three distinct domains: an N-terminal transmembrane domain, a tandem-repeat neck domain and C-type lectin carbohydrate recognition domain. The extracellular region consisting of the C-type lectin and neck domains has a dual function as a pathogen recognition receptor and a cell adhesion receptor by binding carbohydrate ligands on the surface of microbes and endogenous cells. The neck region is important for homo-oligomerization which allows the receptor to bind multivalent ligands with high avidity. Variations in the number of 23 amino acid repeats in the neck domain of this protein are rare but have a significant impact on ligand binding ability. Human DC-SIGN is encoded by the CD209 gene (GeneID 30835) which is closely related in terms of both sequence and function to a neighboring gene (GeneID 10332; often referred to as L-SIGN). DC-SIGN and L-SIGN differ in their ligand-binding properties and distribution. Alternative splicing results in multiple variants. The human CD209 gene is mapped to chromosomal location 19p13.2, and the genomic sequence of CD209 gene can be found in GenBank at NG_012167.1. In human, there are seven DC-SIGN isoforms: 1, 3, 4, 5, 6, 7, and 8; the term “DC-SIGN” is used herein to refer collectively to all DC-SIGN isoforms. As used herein, a human DC-SIGN protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with DC-SIGN isoforms: 1, 3, 4, 5, 6, 7, and 8, wherein such proteins still have at least one of the functions of DC-SIGN. The mRNA and protein sequences for human DC-SIGN isoform 1, the longest isoform, are:

| Homo sapiens CD209 molecule (CD209), transcript variant 1, | |
|---|---|
| mRNA +NM_021155.3+ | |
| (SEQ ID NO: 302) | |
| 1 | atcacagggt gggaaataaa agctgtggcc cccaggagtt ctggacactg ggggagagtg |
| 61 | gggtgacatg agtgactcca aggaaccaag actgcagcag ctgggcctcc tggaggagga |
| 121 | acagctgaga ggccttggat tccgacagac tcgaggatac aagagcttag cagggtgtct |
| 181 | tggccatggt cccctggtgc tgcaactcct ctccttcacg ctcttggctg ggctccttgt |
| 241 | ccaagtgtcc aaggtcccca gctccataag tcaggaacaa tccaggcaag acgcgatcta |
| 301 | ccagaacctg acccagctta aagctgcagt gggtgagctc tcagagaaat ccaagctgca |
| 361 | ggagatctac caggagctga cccagctgaa ggctgcagtg ggtgagcttc cagagaaatc |
| 421 | taagctgcag gagatctacc aggagctgac ccggctgaag gctgcagtgg gtgagcttcc |
| 481 | agagaaatct aagctgcagg agatctacca ggagctgacc tggctgaagg ctgcagtggg |
| 541 | tgagcttcca gagaaatcta agatgcagga gatctaccag gagctgactc ggctgaaggc |
| 601 | tgcagtgggt gagcttccag agaaatctaa gcagcaggag atctaccagg agctgacccg |
| 661 | gctgaaggct gcagtgggtg agcttccaga gaaatctaag cagcaggaga tctaccagga |
| 721 | gctgacccgg ctgaaggctg cagtgggtga gcttccagag aaatctaagc agcaggagat |
| 781 | ctaccaggag ctgacccagc tgaaggctgc agtggaacgc ctgtgccacc cctgtccctg |
| 841 | ggaatggaca ttcttccaag gaaactgtta cttcatgtct aactcccagc ggaactggca |
| 901 | cgactccatc accgcctgca aagaagtggg ggcccagctc gtcgtaatca aaagtgctga |
| 961 | ggagcagaac ttcctacagc tgcagtcttc cagaagtaac cgcttcacct ggatgggact |
| 1021 | ttcagatcta aatcaggaag gcacgtggca atgggtggac ggctcacctc tgttgcccag |
| 1081 | cttcaagcag tattggaaca gaggagagcc caacaacgtt ggggaggaag actgcgcgga |
| 1141 | atttagtggc aatggctgga acgacgacaa atgtaatctt gccaaattct ggatctgcaa |
| 1201 | aaagtccgca gcctcctgct ccagggatga agaacagttt ctttctccag cccctgccac |
| 1261 | cccaaacccc cctcctgcgt agcagaactt cacccccttt taagctacag ttccttctct |
| 1321 | ccatccttcg accttcacaa aatctctggg actgttcttt gtcagattct tcctccttta |
| 1381 | gaaggctggg tcccattctg tccttcttgt catgcctcca atttcccctg gtgtagagct |
| 1441 | tgtttttctg gcccatcctt ggagctttat gagtgagctg gtgtgggatg cctttggggg |
| 1501 | tggacttgtg ttccaagaat ccactctctc ttccttttgg agattaggat atttgggttg |
| 1561 | ccatgtgtag ctgctatgtc ccctggggcg ttatcttata catgcaaacc taccatctgt |
| 1621 | tcaacttcca cctaccacct cctgcacccc tttgatcggg gacttactgg ttgcaagagc |
| 1681 | tcattttgca ggctggaagc accagggaat taattccccc agtcaaccaa tggcacccag |
| 1741 | agagggcatg gaggctccac gcaacccctt ccacccccac atcttccttt gtcttataca |
| 1801 | tggcttccat ttggctgttt ctaagttgta ttctttattt tattattatt attactattt |
| 1861 | ttcgagatgg agtttcactc ttgtcgctca ggctggagtg ccatggcgcg atcttggctc |
| 1921 | actgcaacct ctgcctcccg ggttcaagtg attctcctgc ctcagcctca cgagtagctg |
| 1981 | gaattacagg caggcgccac cagacccggc taattttttg tatttttagt acagatgggg |
| 2041 | tttctccgtg ttggtcaggc tggtcttgaa ctcccgacct cagatgatct gcccgcctcg |
| 2101 | gcctcccaaa attgctggga ttacaggtgt gagccaccgc gcctggccta ttattttttg |
| 2161 | taagaataaa acaggtttat tgggatttgg gactctgaac agttctgtct ctactacctg |
| 2221 | atctcctcct accacgactt tgggatctag aggagctttg gctccggctg tgacggctcc |
| 2281 | ggccgttctc actgcggctg caccggcccc cgctgcggtc actatttctt cctctgctag |
| 2341 | gtgaattgtg cctctcctgg ctctttgaca tgtgctagtg agatttcttc cttttccttt |
| 2401 | cggattcccc atttcttttg taggaatggt ctggactagg gttctccttc cccgcagcct |
| 2461 | gtagtattca tcgtggtggc ccaccctctc tctccccttg gagctcttgc caaaggagga |
| 2521 | gacaagcaga ggtctctatt ggatttctca acacctgaag aaagttgcag tgttttcctc |
| 2581 | ttggacattg ttgtatttca aataaaccac aaatcatcat tttccaccga gccactgggc |
| 2641 | agaattcaca ctgaagctgt cgtcctgcgt acataccatc gtccgttaaa cagagaaaga |
| 2701 | gctgcttggc attcttcttc cgactggtac tgaacatata tacttgcccc tcaggtgagg |
| 2761 | ttccaagttg caactgacct tgaactgaat cactctcccc acgttatttt ttaattacta |
| 2821 | ttttttttta aagatggggt cttgctctgt cgccaggctg gagtgcagtg gcgcgatcta |
| 2881 | ggctcactgc aacttccgcc tcccgggttc aagcgattct cctgcctcag cctcccgagt |
| 2941 | agctgggact ccactaaaag tacaaaaatt agctgggcgt gcaccactgc gcccagctaa |
| 3001 | ttcttgtatt tttggtagag acggggtttc aacatgttga ccaggatggt ctcgatctct |
| 3061 | tgacctcgtg attcgcccgc cgcgtcctcc caaagtgctg ggattacagg cctgagccac |
| 3121 | cgcgcccagt ctctccccac gttcttgaac tcgggcagca catcctcaca gaaatctagg |
| 3181 | aactgttggt aggtttcttc ctcgctgtac tccaggcttg cttcggagtc atagtcatcc |
| 3241 | ctcctgcact gctcctttcc aaacactgta aacatgcttt taataagaag ggtaggactg |
| 3301 | gatgttggga aatcatgtga acatctatct ccaaatctgc aagctcctgt tttactgtag |
| 3361 | aagggacaat taactccatc cttctccatg actctgaaat ccaagggggg gttccgggtt |
| 3421 | ttgccatgtg gcgccatttt ccaactcatt ttcagcctga tccagcatct tctggacagc |
| 3481 | ttccggtttt tgtttcttct gtcgtttctg ttcctcctcc tctctctctt tcctctgctg |
| 3541 | ttcttcccat tgttccttta actttcgctc ttgttcttgc cgttttctag ccacctcttc |
| 3601 | cttttccttc tttattctga attcttcttg tgccttctgc tctctcagca accactcctc |
| 3661 | atgtaatctt tgcctctctc ttccccatag cttttctagt tgttgttttt caataaaagt |
| 3721 | gtcctcctct ttctgtgaga gtcctgagtc cctcagtgga gcaagttcct gctggcgttt |
| 3781 | ctttcgtttc tccttcttca gggcggccct gtactttttg tggcttggtt tctctggaaa |
| 3841 | tgtcaccttt tcgggcgcag ccatcttgcc ggcaccgccc cgcccctcta gttgtatcct |
| 3901 | ttataataaa ctggtaaaca ttgtaaccgc agattcagcc caatctggtt caactttgtg |
| 3961 | taataaaatg gcgagttgtt tttcagttgt cgtggacccc caggttgcaa gttacatacc |
| 4021 | ctgggcatgt ccagatgaac gaagcgtgca aatccacgtg gaacctaagt gctcagaccg |
| 4081 | aggaacaggg actgagttaa gaagtggaca ccacgtggca tgatccttga tccaatcaga |
| 4141 | ttgagccctg gcgtgatcca gtcagatcaa gcctcctgaa tcccctcatt acaagatcca |
| 4201 | atcatatcat gcctcactac cctctgtata taaaatctgc cccagcctcc aacttggaga |
| 4261 | gacagatttg ggccagactc ctgtgtcctt gcttggctgc cttgcaataa atttttctct |
| 4321 | ctacaaaa |
| CD209 antigen isoform 4 sapiens+ +NP_066978.1+ | |
| | (SEQ ID NO: 303) |
| 1 | msdskeprlq qlglleeeql rglgfrqtrg ykslagclgh gplvlqllsf tllagllvqv |
| 61 | skvpssisqe qsrqdaiyqn ltqlkaavge lseksklqei yqeltqlkaa vgelpekskl |
| 121 | qeiyqeltrl kaavgelpek sklqeiyqel twlkaavgel pekskmqeiy qeltrlkaav |
| 181 | gelpekskqq eiyqeltrlk aavgelpeks kqqeiyqelt rlkaavgelp ekskqqeiyq |
| 241 | eltqlkaave rlchpcpwew tffqgncyfm snsqrnwhds itackevgaq lvviksaeeq |
| 301 | nflqlqssrs nrftwmglsd lnqegtwqwv dgspllpsfk qywnrgepnn vgeedcaefs |
| 361 | gngwnddkcn lakfwickks aascsrdeeq flspapatpn pppa |

The mRNA and protein sequences of the other human DC-SIGN isoforms can be found in GeneBank with the following Accession Nos:

DC-SIGN isoform 3: NM_001144896.1 (mRNA)→NP_001138368.1 (protein);

DC-SIGN isoform 4: NM_001144897.1 (mRNA)→NP_001138369.1 (protein);

DC-SIGN isoform 5: NM_001144893.1 (mRNA)→NP_001138365.1 (protein);

DC-SIGN isoform 6: NM_001144894.1 (mRNA)→NP_001138366.1 (protein);

DC-SIGN isoform 7: NM_001144895.1 (mRNA)→NP_001138367.1 (protein);

DC-SIGN isoform 8: NM_001144899.1 (mRNA)→NP_001138371.1 (protein);

All the sequences above are hereby incorporated by reference.

As used herein, “L-SIGN” (liver/lymph node-specific intracellular adhesion molecules-3 grabbing non-integrin, also known as CLEC4M, CD299; LSIGN; CD209L; DCSIGNR; HP10347; DC-SIGN2; DC-SIGNR) refers to a transmembrane receptor and is referred to as L-SIGN because of its expression in the endothelial cells of the lymph nodes and liver. The protein is involved in the innate immune system and recognizes numerous evolutionarily divergent pathogens ranging from parasites to viruses, with a large impact on public health. The protein is organized into three distinct domains: an N-terminal transmembrane domain, a tandem-repeat neck domain and C-type lectin carbohydrate recognition domain. The extracellular region consisting of the C-type lectin and neck domains has a dual function as a pathogen recognition receptor and a cell adhesion receptor by binding carbohydrate ligands on the surface of microbes and endogenous cells. The neck region is important for homo-oligomerization which allows the receptor to bind multivalent ligands with high avidity. Variations in the number of 23 amino acid repeats in the neck domain of this protein are common and have a significant impact on ligand binding ability. This gene is closely related in terms of both sequence and function to a neighboring gene (GeneID 30835; often referred to as DC-SIGN or CD209). DC-SIGN and L-SIGN differ in their ligand-binding properties and distribution. Alternative splicing results in multiple variants. The human L-SIGN is encoded by the CLEC4M gene (GeneID 10332) which is mapped to chromosomal location 19p13.2, and the genomic sequence of CLEC4M gene can be found in GenBank at NG_029190.1. In human, there are nine L-SIGN isoforms: 1, 2, 3, 7, 8, 9, 10, 11, and 12; the term “L-SIGN” is used herein to refer collectively to all L-SIGN isoforms. As used herein, a human L-SIGN protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with L-SIGN isoforms: 1, 2, 3, 7, 8, 9, 10, 11, and 12, wherein such proteins still have at least one of the functions of L-SIGN. The mRNA and protein sequences for human L-SIGN isoform 1, the longest isoform, are:

| Homo sapiens C-type lectin domain family 4 member M (CLEC4M), | |
|---|---|
| transcript variant 1,mRNA +NM_014257.4+ | |
| | (SEQ ID NO: 304) |
| 1 | acccagcttc ctgtttgtct tcctgagaga cagtagattt agaaagtgag gatcagaggg |
| 61 | tggaaaataa aagctgtggt ccccaggagt cctgaacatc tggggacagc gggaaaacat |
| 121 | gagtgactcc aaggaaccaa gggtgcagca gctgggcctc ctggaagaag atccaacaac |
| 181 | cagtggcatc agactttttc caagagactt tcaattccag cagatacatg gccacaagag |
| 241 | ctctacaggg tgtcttggcc atggcgccct ggtgctgcaa ctcctctcct tcatgctctt |
| 301 | ggctggggtc ctggtggcca tccttgtcca agtgtccaag gtccccagct ccctaagtca |
| 361 | ggaacaatcc gagcaagacg caatctacca gaacctgacc cagcttaaag ctgcagtggg |
| 421 | tgagctctca gagaaatcca agctgcagga gatctaccag gagctgaccc agctgaaggc |
| 481 | tgcagtgggt gagttgccag agaaatccaa gctgcaggag atctaccagg agctgacccg |
| 541 | gctgaaggct gcagtgggtg agttgccaga gaaatccaag ctgcaggaga tctaccagga |
| 601 | gctgacccgg ctgaaggctg cagtgggtga gttgccagag aaatccaagc tgcaggagat |
| 661 | ctaccaggag ctgacccggc tgaaggctgc agtgggtgag ttgccagaga aatccaagct |
| 721 | gcaggagatc taccaggagc tgacggagct gaaggctgca gtgggtgagt tgccagagaa |
| 781 | atccaagctg caggagatct accaggagct gacccagctg aaggctgcag tgggtgagtt |
| 841 | gccagaccag tccaagcagc agcaaatcta tcaagaactg accgatttga agactgcatt |
| 901 | tgaacgcctg tgccgccact gtcccaagga ctggacattc ttccaaggaa actgttactt |
| 961 | catgtctaac tcccagcgga actggcacga ctccgtcacc gcctgccagg aagtgagggc |
| 1021 | ccagctcgtc gtaatcaaaa ctgctgagga gcagaacttc ctacagctgc agacttccag |
| 1081 | gagtaaccgc ttctcctgga tgggactttc agacctaaat caggaaggca cgtggcaatg |
| 1141 | ggtggacggc tcacctctgt cacccagctt ccagcggtac tggaacagtg gagaacccaa |
| 1201 | caatagcggg aatgaagact gtgcggaatt tagtggcagt ggctggaacg acaatcgatg |
| 1261 | tgacgttgac aattactgga tctgcaaaaa gcccgcagcc tgcttcagag acgaatagtt |
| 1321 | gtttccctgc tagcctcagc ctccattgtg gtatagcaga acttcaccca cttgtaagcc |
| 1381 | agcgcttctt ctctccatcc ttggaccttc acaaatgccc tgagacggtt ctctgttcga |
| 1441 | tttttcatcc cctatgaacc tgggtcttat tctgtccttc tgatgcctcc aagtttccct |
| 1501 | ggtgtagagc ttgtgttctt ggcccatcct tggagcttta taagtgacct gagtgggatg |
| 1561 | catttagggg gcgggcttgg tatgttgtat gaatccactc tctgttcctt ttggagatta |
| 1621 | gactatttgg attcatgtgt agctgccctg tcccctgggg ctttatctca tccatgcaaa |
| 1681 | ctaccatctg ctcaacttcc agctacaccc cgtgcaccct tttgactggg gacttgctgg |
| 1741 | ttgaaggagc tcatcttgca ggctggaagc accagggaat taattccccc agtcaaccaa |
| 1801 | tggcatccag agagggcatg gaggctccat acaacctctt ccacccccac atctttcttt |
| 1861 | gtcctataca tgtcttccat ttggctgttt ctgagttgta gcctttataa taaagtggta |
| 1921 | aatgttgtaa ctgcaaaaaa aaaaaaaaaa aaaaaaaaaa aaaaaaaaaa aaaaaa |
| C-type lectin domain family 4 member M isoform 1 sapiens+ +NP_055072.3+ | |
| | (SEQ ID NO: 305) |
| 1 | msdskeprvq qlglleedpt tsgirlfprd fqfqqihghk sstgclghga lvlqllsfml |
| 61 | lagvlvailv qvskvpssls qeqseqdaiy qnitqlkaav gelseksklq eiyqeltqlk |
| 121 | aavgelpeks klqeiyqelt rlkaavgelp eksklqeiyq eltrlkaavg elpeksklqe |
| 181 | iyqeltrlka avgelpeksk lqeiyqelte lkaavgelpe ksklqeiyqe ltqlkaavge |
| 241 | lpdqskqqqi yqeltdlkta ferlcrhcpk dwtffqgncy fmsnsqrnwh dsvtacqevr |
| 301 | aqlvvktae eqnflqlqts rsnrfswmgl sdlnqegtwq wydgsplsps fqrywnsgep |
| 361 | nnsgnedcae fsgsgwndnr cdvdnywick kpaacfrde |

The mRNA and protein sequences of the other human L-SIGN isoforms can be found in GeneBank with the following Accession Nos:

L-SIGN isoform 2: NM_001144904.1 (mRNA)→NP_001138376.1 (protein);

L-SIGN isoform 3: NP_001138382.1 (mRNA)→NP_001138383.1 (protein);

L-SIGN isoform 7: NM_001144906.1 (mRNA)→NP_001138378.1 (protein);

L-SIGN isoform 8: NM_001144910.1 (mRNA)→NP_001138382.1 (protein);

L-SIGN isoform 9: NM_001144909.1 (mRNA)→NP_001138381.1 (protein);

L-SIGN isoform 10: NM_001144908.1 (mRNA)→NP_001138380.1 (protein);

L-SIGN isoform 11: NM_001144907.1 (mRNA)→NP_001138379.1 (protein);

L-SIGN isoform 12: NM_001144905.1 (mRNA)→NP_001138377.1 (protein);

All the sequences above are hereby incorporated by reference.

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

The term “antibody fragment” or “antigen-binding fragment” or “functional fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof, and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). In a combined Kabat and Chothia numbering scheme for a given CDR region (for example, HC CDR1, HC CDR2, HC CDR3, LC CDR1, LC CDR2 or LC CDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.”

For example, under Kabat, the CDR amino acid residues in the heavy chain variable domain (VH) are numbered 31-35 (HCDR1) (e.g., insertion(s) after position 35), 50-65 (HCDR2), and 95-102 (HCDR3); and the CDR amino acid residues in the light chain variable domain (VL) are numbered 24-34 (LCDR1) (e.g., insertion(s) after position 27), 50-56 (LCDR2), and 89-97 (LCDR3). As another example, under Chothia, the CDR amino acids in the VH are numbered 26-32 (HCDR1) (e.g., insertion(s) after position 31), 52-56 (HCDR2), and 95-102 (HCDR3); and the amino acid residues in VL are numbered 26-32 (LCDR1) (e.g., insertion(s) after position 30), 50-52 (LCDR2), and 91-96 (LCDR3). By combining the CDR definitions of both Kabat and Chothia, the CDRs comprise or consist of, e.g., amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.” Conformational and linear epitopes are distinguished in that the binding to the former but not the latter is lost in the presence of denaturing solvents.

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody,” as used herein, includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia, and ImMunoGenTics (IMGT) numbering (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; Al-Lazikani et al., (1997) J. Mal. Biol. 273:927-948; and Lefranc, M.-P., The Immunologist, 7, 132-136 (1999); Lefranc, M.-P. et al., Dev. Comp. Immunol., 27, 55-77 (2003)).

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

The term “binding specificity” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within an antibody can be replaced with other amino acid residues from the same side chain family and the altered antibody can be tested using the functional assays described herein.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See www.ncbi.nlm.nih.gov.

The terms “cancer” and “cancerous” refer to or describe the physiological condition in mammals that is typically characterized by unregulated cell growth. Examples of cancer include, but are not limited to, solid tumors and hematological cancers, including carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric cancer including gastrointestinal cancer, pancreatic cancer, glioblastoma, neuroblastoma, cervical cancer, ovarian cancer, liver cancer, bladder cancer, urinary tract cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. Additional cancer indications are disclosed herein.

The terms “tumor antigen” or “cancer associated antigen” interchangeably refer to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy.

The terms “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. The tumor-supporting antigen itself need not play a role in supporting the tumor cells so long as the antigen is present on a cell that supports cancer cells.

The terms “combination” or “pharmaceutical combination,” as used herein mean a product that results from the mixing or combining of more than one active ingredient and includes both fixed and non-fixed combinations of the active ingredients. The term “fixed combination” means that the active ingredients, by way of example, a compound of the invention and one or more additional therapeutic agent, are administered to a subject simultaneously in the form of a single entity or dosage. The term “non-fixed combination” means that the active ingredients, by way of example, a compound of of the invention and one or more additional therapeutic agent, are administered to a subject as separate entities either simultaneously, concurrently or sequentially with no specific time limits, wherein such administration provides therapeutically effective levels of the active ingredients in the body of the subject. The latter also applies to cocktail therapy, e.g. the administration of 3 or more active ingredients.

The terms “composition” or “pharmaceutical composition,” as used herein, refers to a mixture of a compound of the invention with at least one and optionally more than one other pharmaceutically acceptable chemical components, such as carriers, stabilizers, diluents, dispersing agents, suspending agents, thickening agents, and/or excipients.

The term “an optical isomer” or “a stereoisomer”, as used herein, refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

The term “pharmaceutically acceptable carrier”, as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “pharmaceutically acceptable salt,” as used herein, refers to a salt which does not abrogate the biological activity and properties of the compounds of the invention, and does not cause significant irritation to a subject to which it is administered.

The term “subject”, as used herein, encompasses mammals and non-mammals. Examples of mammals include, but are not limited to, humans, chimpanzees, apes, monkeys, cattle, horses, sheep, goats, swine; rabbits, dogs, cats, rats, mice, guinea pigs, and the like. Examples of non-mammals include, but are not limited to, birds, fish and the like. Frequently the subject is a human.

The term “a subject in need of such treatment”, refers to a subject which would benefit biologically, medically or in quality of life from such treatment.

The term “STING” refers to STtimulator of INterferon Genes receptor, also known as TMEM173, ERIS, MITA, MPYS, SAVI, or NET23). As used herein, the terms “STING” and “STING receptor” are used interchangeably, and include different isoforms and variants of STING. The mRNA and protein sequences for human STING isoform 1, the longest isoform, are:

| Homo sapiens transmembrane protein 173 (TMEM173), transcript variant 1, | |
|---|---|
| mRNA +NM_198282.3+ | |
| | [SEQ ID NO: 932] |
| 1 | tataaaaata gctcttgtta ccggaaataa ctgttcattt ttcactcctc cctcctaggt |
| 61 | cacacttttc agaaaaagaa tctgcatcct ggaaaccaga agaaaaatat gagacgggga |
| 121 | atcatcgtgt gatgtgtgtg ctgcctttgg ctgagtgtgt ggagtcctgc tcaggtgtta |
| 181 | ggtacagtgt gtttgatcgt ggtggcttga ggggaacccg ctgttcagag ctgtgactgc |
| 241 | ggctgcactc agagaagctg cccttggctg ctcgtagcgc cgggccttct ctcctcgtca |
| 301 | tcatccagag cagccagtgt ccgggaggca gaagatgccc cactccagcc tgcatccatc |
| 361 | catcccgtgt cccaggggtc acggggccca gaaggcagcc ttggttctgc tgagtgcctg |
| 421 | cctggtgacc ctttgggggc taggagagcc accagagcac actctccggt acctggtgct |
| 481 | ccacctagcc tccctgcagc tgggactgct gttaaacggg gtctgcagcc tggctgagga |
| 541 | gctgcgccac atccactcca ggtaccgggg cagctactgg aggactgtgc gggcctgcct |
| 601 | gggctgcccc ctccgccgtg gggccctgtt gctgctgtcc atctatttct actactccct |
| 661 | cccaaatgcg gtcggcccgc ccttcacttg gatgcttgcc ctcctgggcc tctcgcaggc |
| 721 | actgaacatc ctcctgggcc tcaagggcct ggccccagct gagatctctg cagtgtgtga |
| 781 | aaaagggaat ttcaacgtgg cccatgggct ggcatggtca tattacatcg gatatctgcg |
| 841 | gctgatcctg ccagagctcc aggcccggat tcgaacttac aatcagcatt acaacaacct |
| 901 | gctacggggt gcagtgagcc agcggctgta tattctcctc ccattggact gtggggtgcc |
| 961 | tgataacctg agtatggctg accccaacat tcgcttcctg gataaactgc cccagcagac |
| 1021 | cggtgaccat gctggcatca aggatcgggt ttacagcaac agcatctatg agcttctgga |
| 1081 | gaacgggcag cgggcgggca cctgtgtcct ggagtacgcc acccccttgc agactttgtt |
| 1141 | tgccatgtca caatacagtc aagctggctt tagccgggag gataggcttg agcaggccaa |
| 1201 | actcttctgc cggacacttg aggacatcct ggcagatgcc cctgagtctc agaacaactg |
| 1261 | ccgcctcatt gcctaccagg aacctgcaga tgacagcagc ttctcgctgt cccaggaggt |
| 1321 | tctccggcac ctgcggcagg aggaaaagga agaggttact gtgggcagct tgaagacctc |
| 1381 | agcggtgccc agtacctcca cgatgtccca agagcctgag ctcctcatca gtggaatgga |
| 1441 | aaagcccctc cctctccgca cggatttctc ttgagaccca gggtcaccag gccagagcct |
| 1501 | ccagtggtct ccaagcctct ggactggggg ctctcttcag tggctgaatg tccagcagag |
| 1561 | ctatttcctt ccacaggggg ccttgcaggg aagggtccag gacttgacat cttaagatgc |
| 1621 | gtcttgtccc cttgggccag tcatttcccc tctctgagcc tcggtgtctt caacctgtga |
| 1681 | aatgggatca taatcactgc cttacctccc tcacggttgt tgtgaggact gagtgtgtgg |
| 1741 | aagtttttca taaactttgg atgctagtgt acttaggggg tgtgccaggt gtctttcatg |
| 1801 | gggccttcca gacccactcc ccacccttct ccccttcctt tgcccgggga cgccgaactc |
| 1861 | tctcaatggt atcaacaggc tccttcgccc tctggctcct ggtcatgttc cattattggg |
| 1921 | gagccccagc agaagaatgg agaggaggag gaggctgagt ttggggtatt gaatcccccg |
| 1981 | gctcccaccc tgcagcatca aggttgctat ggactctcct gccgggcaac tcttgcgtaa |
| 2041 | tcatgactat ctctaggatt ctggcaccac ttccttccct ggccccttaa gcctagctgt |
| 2101 | gtatcggcac ccccacccca ctagagtact ccctctcact tgcggtttcc ttatactcca |
| 2161 | cccctttctc aacggtcctt ttttaaagca catctcagat tacccaaaaa aaaaaaaaaa |
| 2221 | aaa |

Homo sapiens stimulator of interferon genes protein isoform 1 +NP_938023.1+
[SEQ ID NO: 933]
MPHSSLHPSIPCPRGHGAQKAALVLLSACLVTLWGLGEPPEHTLRYLVL
HLASLQLGLLLNGVCSLAEELRHIHSRYRGSYWRTVRACLGCPLRRGAL
LLLSIYFYYSLPNAVGPPFTWMLALLGLSQALNILLGLKGLAPAEISAV
CEKGNFNVAHGLAWSYYIGYLRLILPELQARIRTYNQHYNNLLRGAVSQ
RLYILLPLDCGVPDNLSMADPNIRFLDKLPQQTGDHAGIKDRVYSNSIY
ELLENGQRAGTCVLEYATPLQTLFAMSQYSQAGFSREDRLEQAKLFCRT
LEDILADAPESQNNCRLIAYQEPADDSSFSLSQEVLRHLRQEEKEEVTV
GSLKTSAVPSTSTMSQEPELLISGMEKPLPLRTDFS

The mRNA and protein sequences for human STING isoform 2, a shorter isoform, are:

| Homo sapiens transmembrane protein 173 (TMEM173), transcript variant 2, | |
|---|---|
| mRNA +NM_001301738.1+ | |
| [SEQ ID NO: 934] | |
| 1 | gctgcactca gagaagctgc ccttggctgc tcgtagcgcc gggccttctc tcctcgtcat |
| 61 | catccagagc agccagtgtc cgggaggcag aagatgcccc actccagcct gcatccatcc |
| 121 | atcccgtgtc ccaggggtca cggggcccag aaggcagcct tggttctgct gagtgcctgc |
| 181 | ctggtgaccc tttgggggct aggagagcca ccagagcaca ctctccggta cctggtgctc |
| 241 | cacctagcct ccctgcagct gggactgctg ttaaacgggg tctgcagcct ggctgaggag |
| 301 | ctgcgccaca tccactccag gtaccggggc agctactgga ggactgtgcg ggcctgcctg |
| 361 | ggctgccccc tccgccgtgg ggccctgttg ctgctgtcca tctatttcta ctactccctc |
| 421 | ccaaatgcgg tcggcccgcc cttcacttgg atgcttgccc tcctgggcct ctcgcaggca |
| 481 | ctgaacatcc tcctgggcct caagggcctg gccccagctg agatctctgc agtgtgtgaa |
| 541 | aaagggaatt tcaacgtggc ccatgggctg gcatggtcat attacatcgg atatctgcgg |
| 601 | ctgatcctgc cagagctcca ggcccggatt cgaacttaca atcagcatta caacaacctg |
| 661 | ctacggggtg cagtgagcca gcggctgtat attctcctcc cattggactg tggggtgcct |
| 721 | gataacctga gtatggctga ccccaacatt cgcttcctgg ataaactgcc ccagcagacc |
| 781 | ggtgaccatg ctggcatcaa ggatcgggtt tacagcaaca gcatctatga gcttctggag |
| 841 | aacgggcagc ggaacctgca gatgacagca gcttctcgct gtcccaggag gttctccggc |
| 901 | acctgcggca ggaggaaaag gaagaggtta ctgtgggcag cttgaagacc tcagcggtgc |
| 961 | ccagtacctc cacgatgtcc caagagcctg agctcctcat cagtggaatg gaaaagcccc |
| 1021 | tccctctccg cacggatttc tcttgagacc cagggtcacc aggccagagc ctccagtggt |
| 1081 | ctccaagcct ctggactggg ggctctcttc agtggctgaa tgtccagcag agctatttcc |
| 1141 | ttccacaggg ggccttgcag ggaagggtcc aggacttgac atcttaagat gcgtcttgtc |
| 1201 | cccttgggcc agtcatttcc cctctctgag cctcggtgtc ttcaacctgt gaaatgggat |
| 1261 | cataatcact gccttacctc cctcacggtt gttgtgagga ctgagtgtgt ggaagttttt |
| 1321 | cataaacttt ggatgctagt gtacttaggg ggtgtgccag gtgtctttca tggggccttc |
| 1381 | cagacccact ccccaccctt ctccccttcc tttgcccggg gacgccgaac tctctcaatg |
| 1441 | gtatcaacag gctccttcgc cctctggctc ctggtcatgt tccattattg gggagcccca |
| 1501 | gcagaagaat ggagaggagg aggaggctga gtttggggta ttgaatcccc cggctcccac |
| 1561 | cctgcagcat caaggttgct atggactctc ctgccgggca actcttgcgt aatcatgact |
| 1621 | atctctagga ttctggcacc acttccttcc ctggcccctt aagcctagct gtgtatcggc |
| 1681 | acccccaccc cactagagta ctccctctca cttgcggttt ccttatactc cacccctttc |
| 1741 | tcaacggtcc ttttttaaag cacatctcag attacccaaa aaaaaaaaaa aaaaa |

Homo sapiens stimulator of interferon genes protein isoform 2 +NP_001288667.1+
[SEQ ID NO: 935]
MPHSSLHPSIPCPRGHGAQKAALVLLSACLVTLWGLGEPPEHTLRYLVL
HLASLQLGLLLNGVCSLAEELRHIHSRYRGSYWRTVRACLGCPLRRGAL
LLLSIYFYYSLPNAVGPPFTWMLALLGLSQALNILLGLKGLAPAEISAV
CEKGNFNVAHGLAWSYYIGYLRLILPELQARIRTYNQHYNNLLRGAVSQ
RLYILLPLDCGVPDNLSMADPNIRFLDKLPQQTGDHAGIKDRVYSNSIY
ELLENGQRNLQMTAASRCPRRFSGTCGRRKRKRLLWAA

The sequences of other human STING isoforms/SNPs (single nucleotide polymorphisms) include the following and those described in Yi, PLoS One. 2013 Oct. 21; 8(10):e77846.

hSTING wt (wild type): Reference SNP (refSNP)
Cluster Report: rs1131769
[SEQ ID NO: 936]
atgccccactccagcctgcatccatccatcccgtgtcccaggggtcacg
gggcccagaaggcagccttggttctgctgagtgcctgcctggtgaccct
ttgggggctaggagagccaccagagcacactctccggtacctggtgctc
cacctagcctccctgcagctgggactgctgttaaacggggtctgcagcc
tggctgaggagctgcgccacatccactccaggtaccggggcagctactg
gaggactgtgcgggcctgcctgggctgccccctccgccgtggggccctg
ttgctgctgtccatctatttctactactccctcccaaatgcggtcggcc
cgcccttcacttggatgcttgccctcctgggcctctcgcaggcactgaa
catcctcctgggcctcaagggcctggccccagctgagatctctgcagtg
tgtgaaaaagggaatttcaacgtggcccatgggctggcatggtcatatt
acatcggatatctgcggctgatcctgccagagctccaggcccggattcg
aacttacaatcagcattacaacaacctgctacggggtgcagtgagccag
cggctgtatattctcctcccattggactgtggggtgcctgataacctga
gtatggctgaccccaacattcgcttcctggataaactgccccagcagac
cggtgaccgtgctggcatcaaggatcgggtttacagcaacagcatctat
gagcttctggagaacgggcagcgggcgggcacctgtgtcctggagtacg
ccacccccttgcagactttgtttgccatgtcacaatacagtcaagctgg
ctttagccgggaggataggcttgagcaggccaaactcttctgccggaca
cttgaggacatcctggcagatgcccctgagtctcagaacaactgccgcc
tcattgcctaccaggaacctgcagatgacagcagcttctcgctgtccca
ggaggttctccggcacctgcggcaggaggaaaaggaagaggttactgtg
ggcagcttgaagacctcagcggtgcccagtacctccacgatgtcccaag
agcctgagctcctcatcagtggaatggaaaagcccctccctctccgcac
ggatttctcttga
hSTING R293Q: Reference SNP (refSNP) Cluster
Report: rs1131769 rs7380824
[SEQ ID NO: 937]
atgccccactccagcctgcatccatccatcccgtgtcccaggggtcacg
gggcccagaaggcagccttggttctgctgagtgcctgcctggtgaccct
ttgggggctaggagagccaccagagcacactctccggtacctggtgctc
cacctagcctccctgcagctgggactgctgttaaacggggtctgcagcc
tggctgaggagctgcgccacatccactccaggtaccggggcagctactg
gaggactgtgcgggcctgcctgggctgccccctccgccgtggggccctg
ttgctgctgtccatctatttctactactccctcccaaatgcggtcggcc
cgcccttcacttggatgcttgccctcctgggcctctcgcaggcactgaa
catcctcctgggcctcaagggcctggccccagctgagatctctgcagtg
tgtgaaaaagggaatttcaacgtggcccatgggctggcatggtcatatt
acatcggatatctgcggctgatcctgccagagctccaggcccggattcg
aacttacaatcagcattacaacaacctgctacggggtgcagtgagccag
cggctgtatattctcctcccattggactgtggggtgcctgataacctga
gtatggctgaccccaacattcgcttcctggataaactgccccagcagac
cggtgaccgtgctggcatcaaggatcgggtttacagcaacagcatctat
gagcttctggagaacgggcagcgggcgggcacctgtgtcctggagtacg
ccacccccttgcagactttgtttgccatgtcacaatacagtcaagctgg
ctttagccgggaggataggcttgagcaggccaaactcttctgccagaca
cttgaggacatcctggcagatgcccctgagtctcagaacaactgccgcc
tcattgcctaccaggaacctgcagatgacagcagcttctcgctgtccca
ggaggttctccggcacctgcggcaggaggaaaaggaagaggttactgtg
ggcagcttgaagacctcagcggtgcccagtacctccacgatgtcccaag
agcctgagctcctcatcagtggaatggaaaagcccctccctctccgcac
ggatttctcttga
hSTING G230A/R293Q: Reference SNP (refSNP)
ClusterReport:rs1131769 rs7380824 rs78233829
[SEQ ID NO: 938]
atgccccactccagcctgcatccatccatcccgtgtcccaggggtcacg
gggcccagaaggcagccttggttctgctgagtgcctgcctggtgaccct
ttgggggctaggagagccaccagagcacactctccggtacctggtgctc
cacctagcctccctgcagctgggactgctgttaaacggggtctgcagcc
tggctgaggagctgcgccacatccactccaggtaccggggcagctactg
gaggactgtgcgggcctgcctgggctgccccctccgccgtggggccctg
ttgctgctgtccatctatttctactactccctcccaaatgcggtcggcc
cgcccttcacttggatgcttgccctcctgggcctctcgcaggcactgaa
catcctcctgggcctcaagggcctggccccagctgagatctctgcagtg
tgtgaaaaagggaatttcaacgtggcccatgggctggcatggtcatatt
acatcggatatctgcggctgatcctgccagagctccaggcccggattcg
aacttacaatcagcattacaacaacctgctacggggtgcagtgagccag
cggctgtatattctcctcccattggactgtggggtgcctgataacctga
gtatggctgaccccaacattcgcttcctggataaactgccccagcagac
cgctgaccgtgctggcatcaaggatcgggtttacagcaacagcatctat
gagcttctggagaacgggcagcgggcgggcacctgtgtcctggagtacg
ccacccccttgcagactttgtttgccatgtcacaatacagtcaagctgg
ctttagccgggaggataggcttgagcaggccaaactcttctgccagaca
cttgaggacatcctggcagatgcccctgagtctcagaacaactgccgcc
tcattgcctaccaggaacctgcagatgacagcagcttctcgctgtccca
ggaggttctccggcacctgcggcaggaggaaaaggaagaggttactgtg
ggcagcttgaagacctcagcggtgcccagtacctccacgatgtcccaag
agcctgagctcctcatcagtggaatggaaaagcccctccctctccgcac
ggatttctcttga
hSTING R71H/G230A/R293Q: Reference SNP (refSNP)
Cluster Report:rs1131769 rs7380824 rs78233829
rs11554776
[SEQ ID NO: 939]
atgccccactccagcctgcatccatccatcccgtgtcccaggggtcacg
gggcccagaaggcagccttggttctgctgagtgcctgcctggtgaccct
ttgggggctaggagagccaccagagcacactctccggtacctggtgctc
cacctagcctccctgcagctgggactgctgttaaacggggtctgcagcc
tggctgaggagctgcaccacatccactccaggtaccggggcagctactg
gaggactgtgcgggcctgcctgggctgccccctccgccgtggggccctg
ttgctgctgtccatctatttctactactccctcccaaatgcggtcggcc
cgcccttcacttggatgcttgccctcctgggcctctcgcaggcactgaa
catcctcctgggcctcaagggcctggccccagctgagatctctgcagtg
tgtgaaaaagggaatttcaacgtggcccatgggctggcatggtcatatt
acatcggatatctgcggctgatcctgccagagctccaggcccggattcg
aacttacaatcagcattacaacaacctgctacggggtgcagtgagccag
cggctgtatattctcctcccattggactgtggggtgcctgataacctga
gtatggctgaccccaacattcgcttcctggataaactgccccagcagac
cgctgaccgtgctggcatcaaggatcgggtttacagcaacagcatctat
gagcttctggagaacgggcagcgggcgggcacctgtgtcctggagtacg
ccacccccttgcagactttgtttgccatgtcacaatacagtcaagctgg
ctttagccgggaggataggcttgagcaggccaaactcttctgccagaca
cttgaggacatcctggcagatgcccctgagtctcagaacaactgccgcc
tcattgcctaccaggaacctgcagatgacagcagcttctcgctgtccca
ggaggttctccggcacctgcggcaggaggaaaaggaagaggttactgtg
ggcagcttgaagacctcagcggtgcccagtacctccacgatgtcccaag
agcctgagctcctcatcagtggaatggaaaagcccctccctctccgcac
ggatttctcttga

The term “STING agonist”, as used herein, refers to a compound or antibody conjugate capable of binding to STING and activating STING. Activation of STING activity may include, for example, stimulation of inflammatory cytokines, including interferons, such as type 1 interferons, including IFN-α, IFN-β, type 3 interferons, e.g., IFNλ, IP10, TNF, IL-6, CXCL9, CCL4, CXCL11, CCL5, CCL3, or CCL8. STING agonist activity may also include stimulation of TANK binding kinase (TBK) 1 phosphorylation, interferon regulatory factor (IRF) activation (e.g., IRF3 activation), secretion of interferon-γ-inducible protein (IP-10), or other inflammatory proteins and cytokines. STING Agonist activity may be determined, for example, by the ability of a compound to stimulate activation of the STING pathway as detected using an interferon stimulation assay, a reporter gene assay (e.g., a hSTING wt assay, or a THP-1 Dual assay), a TBK1 activation assay, IP-10 assay, a STING Biochemical [3H]cGAMP Competition Assay, or other assays known to persons skilled in the art. STING Agonist activity may also be determined by the ability of a compound to increase the level of transcription of genes that encode proteins activated by STING or the STING pathway. Such activity may be detected, for example, using an RNAseq assay. In some embodiments, an assay to test for activity of a compound in a STING knock-out cell line may be used to determine if the compound is specific for STING, wherein a compound that is specific for STING would not be expected to have activity in a cell line wherein the STING pathway is partially or wholly deleted.

As used herein, the terms “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both.

As used herein, the term “prevent”, “preventing” or “prevention” of any disease or disorder refers to the prophylactic treatment of the disease or disorder; or delaying the onset or progression of the disease or disorder

The term “therapeutically effective amount” or “therapeutically effective dose” interchangeably refers to an amount sufficient to effect the desired result (i.e., reduction or inhibition of an enzyme or a protein activity, amelioration of symptoms, alleviation of symptoms or conditions, delay of disease progression, a reduction in tumor size, inhibition of tumor growth, prevention of metastasis, inhibition or prevention of viral, bacterial, fungal or parasitic infection).

In some embodiments, a therapeutically effective amount does not induce or cause undesirable side effects. In some embodiments, a therapeutically effective amount induces or causes side effects but only those that are acceptable by the healthcare providers in view of a patient's condition. A therapeutically effective amount can be determined by first administering a low dose, and then incrementally increasing that dose until the desired effect is achieved. A “prophylactically effective dose” or a “prophylactically effect amount”, of the molecules of the invention can prevent the onset of disease symptoms, including symptoms associated with cancer. A “therapeutically effective dose” or a “therapeutically effective amount” of the molecules of the invention can result in a decrease in severity of disease symptoms, including symptoms associated with cancer. The compound names provided herein were obtained using ChemDraw Ultra version 14.0 (CambridgeSoft®).

As used herein, the term “a,” “an,” “the” and similar terms used in the context of the present invention (especially in the context of the claims) are to be construed to cover both the singular and plural unless otherwise indicated herein or clearly contradicted by the context.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

Unless specified otherwise, the conjugates or Drug moieties of the present invention refer to compounds of any of formulae (AA-a) through (FF-g) or formulae (A) through (F) or subformulae thereof and exemplified compounds, and salts thereof, as well as all stereoisomers (including diastereoisomers and enantiomers), rotamers, tautomers and isotopically labeled compounds (including deuterium substitutions), as well as inherently formed moieties.

### Immunostimulatory Compounds of the Invention

### Drug Moiety (D)

The Drug moiety (D) of the immunoconjugates of the invention is a compound which binds to Stimulator of Interferon Genes (STING) receptor and which comprises one or more reactive moieties each of which is capable of forming a covalent bond with a linker (L). In one aspect, Drug moiety (D) of the immunoconjugates of the invention is a dinucleotide which binds to Stimulator of Interferon Genes (STING) which comprises one or more reactive moieties capable of forming a covalent bond with a linker (L).

In one aspect, Drug moiety (D) of the immunoconjugates of the invention is a cyclic dinucleotide which binds to Stimulator of Interferon Genes (STING) which comprises one or more reactive moieties capable of forming a covalent bond with a linker (L).

In one aspect the Drug moiety (D) of the immunoconjugates of the invention is a compound having the structure of Formula (A), Formula (B), Formula (C), Formula (D), Formula (E), or Formula (F) or stereoisomers or pharmaceutically acceptable salts thereof,

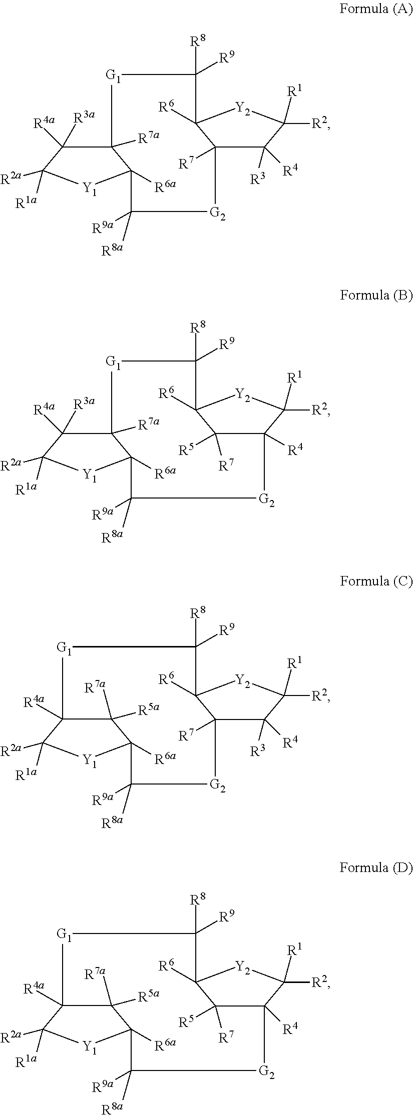

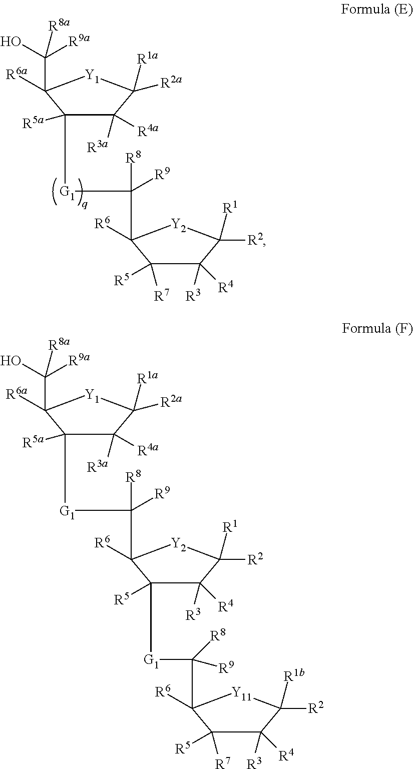

wherein:
each G₁ is independently selected from

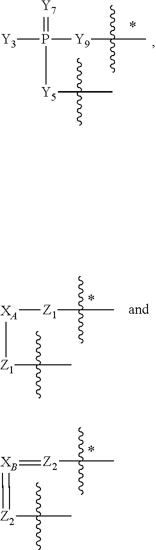

where the * of G₁ indicates the point of attachment to —CR⁸R⁹—;
X_{A} is C(═O)—, —C(═S)— or —C(═NR¹¹)— and each Z₁ is NR¹²;
X_{B} is C, and each Z₂ is N;
G₂ is

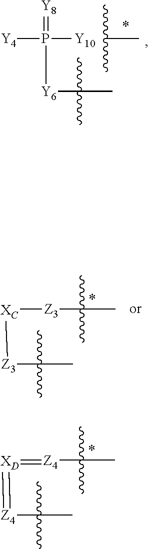

where the * of G₂ indicates the point of attachment to —CR^{8a}R^{9a}—;
X_{C} is C(═O)—, —C(═S)— or —C(═NR¹¹)— and each Z₃ is NR¹²;
X_{D} is C, and each Z₄ is N;
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₃ is OH, O⁻, OR¹⁰, N(R¹⁰)₂, SR¹⁰, SeH, Se⁻, BH₃, SH or S⁻;
Y₄ is OH, O⁻, OR¹⁰, N(R¹⁰)₂, SR¹⁰, SeH, Se⁻, BH₃, SH or S⁻;
Y₅ is —CH₂—, —NH—, —O— or —S;
Y₆ is —CH₂—, —NH—, —O— or —S;
Y₇ is O or S;
Y₈ is O or S;
Y₉ is —CH₂—, —NH—, —O— or —S;
Y₁₀ is —CH₂—, —NH—, —O— or —S;
Y₁₁ is —O—, —S—, —S(═O)—, —SO₂—, —CH₂—, or —CF₂—;
q is 1, 2 or 3;
R¹ is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R¹ is substituted with 0, 1, 2, 3 or 4 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1a} is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1a} is substituted with 0, 1, 2, 3 or 4 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1b} is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1b} is substituted with 0, 1, 2, 3 or 4 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
each R⁸ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁸ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R⁹ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁹ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁹ are substituted by 0, 1, 2 or 3 substituents independently selected from F, Cl, Br, I, OH, CN, and N₃;
R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
R^{3a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
R^{4a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
R^{5a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
R^{7a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
R^{8a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{8a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
R^{9a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{9a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{9a} are substituted by 0, 1, 2 or 3 substituents independently selected from F, Cl, Br, I, OH, CN, and N₃;
each R¹⁰ is independently selected from the group consisting of H, C₁-C₁₂alkyl, C₁-C₆heteroalkyl, —(CH₂CH₂O)ₙCH₂CH₂C(═O)OC₁-C₆alkyl, and

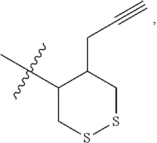 [SMILES: *C1CSSCC1CC#C]

wherein the C₁-C₁₂alkyl and C₁-C₆heteroalkyl of R¹⁰ is substituted by 0, 1, 2 or 3 substituents independently selected from —OH, C₁-C₁₂alkoxy, —S—C(═O)C₁-C₆alkyl, halo, —CN, C₁-C₁₂alkyl, —O-aryl, _O-heteroaryl, —O-cycloalkyl, oxo, cycloalkyl, heterocyclyl, aryl, or heteroaryl, —OC(O)OC₁-C₆alkyland C(O)OC₁-C₆alkyl, wherein each alkyl, cycloalkyl, heterocyclyl, aryl, and heteroaryl is substituted by 0, 1, 2 or 3 substituents independently selected from C₁-C₁₂ alkyl, O—C₁-C₁₂alkyl, C₁-C₁₂heteroalkyl, halo, CN, OH, oxo, aryl, heteroaryl, O-aryl, O-heteroaryl, —C(═O)C₁-C₁₂alkyl, —OC(═O)C₁-C₁₂alkyl, —C(═O)OC₁-C₁₂alkyl, —OC(═O)OC₁-C₁₂alkyl, —C(═O)N(R¹¹)—C₁-C₁₂alkyl, —N(R¹¹)C(═O)—C₁-C₁₂alkyl; —OC(═O)N(R¹¹)—C₁-C₁₂alkyl, —C(═O)-aryl, —C(═O)-heteroaryl, —OC(═O)-aryl, —C(═O)O-aryl, —OC(═O)-heteroaryl, —C(═O)O-heteroaryl, —C(═O)O-aryl, —C(═O)O-heteroaryl, —C(═O)N(R¹¹)-aryl, —C(═O)N(R¹¹)-heteroaryl, —N(R¹¹)C(O)-aryl, —N(R¹¹)₂C(O)-aryl, —N(R¹¹)C(O)-heteroaryl, and S(O)₂N(R¹¹)-aryl;
each R¹¹ is independently selected from H and C₁-C₆alkyl;
each R¹² is independently selected from H and C₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position;
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position;
   optionally R⁸ and R⁹ are connected to form a C₁-C₆alkylene, C₂-C₆alkenylene, C₂-C₆alkynylene, and
   optionally R^{8a} and R^{9a} are connected to form a C₁-C₆alkylene, C₂-C₆alkenylene, C₂-C₆alkynylene.

Certain aspects and examples of compounds which can be incorporated as a Drug moiety (D) in the immunoconjugates of the invention are provided in the following listing of additional, enumerated embodiments. It will be recognized that features specified in each embodiment may be combined with other specified features to provide further embodiments of the present invention.

### Embodiment 1

A compound of Formula (A-1), Formula (B-1), Formula (C-1), Formula (D-1), Formula (E-1) or Formula (F-1), or stereoisomers or pharmaceutically acceptable salts thereof,

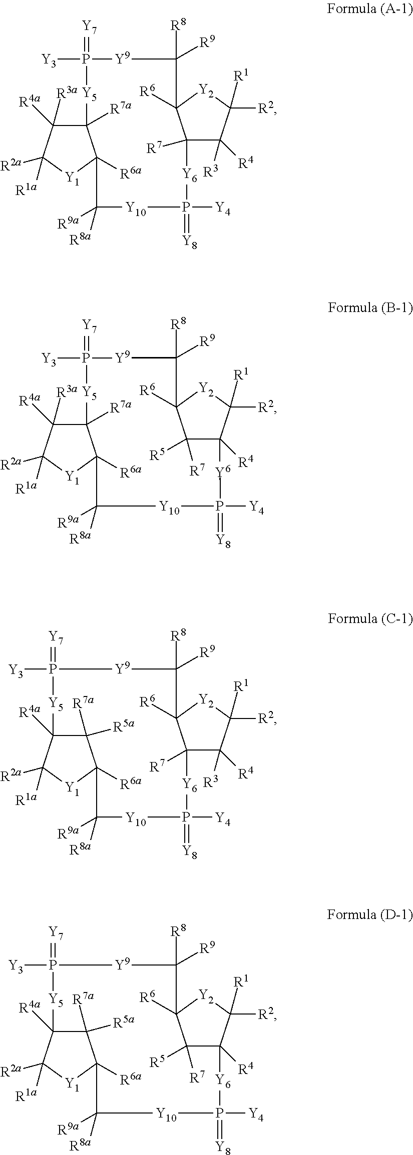

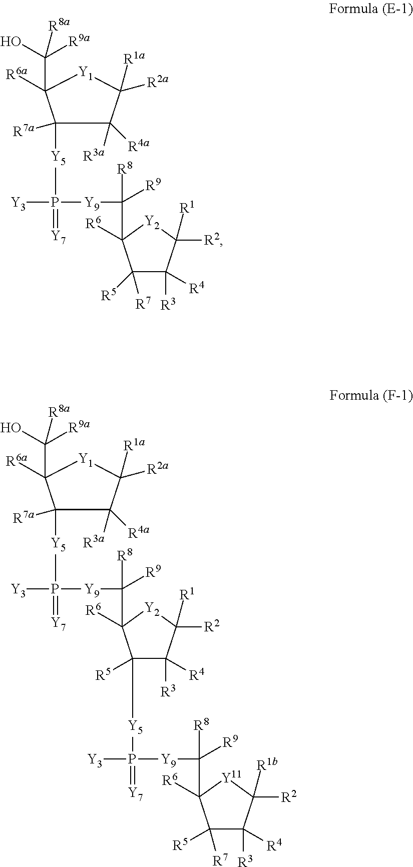

wherein R¹, R^{1a}, R^{1b}, R², R^{2a}, R³, R^{3a}, R⁴, R^{4a}, R⁵, R^{5a}, R⁶, R^{6a}, R⁷, R^{7a}, R⁸, R^{8a}, R⁹, Y₁, Y₂, Y₃, Y₄, Y₅, Y₆, Y₇, Y₈, Y₉, Y₁₀ and Y₁₁ are as defined above for compounds of Formula (A), Formula (B), Formula (C), Formula (D), Formula (E) and Formula (F).

### Embodiment 2

A compound of Formula (A), Formula (B), Formula (C), Formula (D), Formula (A-1), Formula (B-1), Formula (C-1), Formula (D-1), Formula (E-1), or Formula (F-1), wherein R¹ is pyrimidine or purine nucleic acid base or analogue thereof, R^{1a} is pyrimidine or purine nucleic acid base or analogue thereof, and R^{1b} is a pyrimidine or purine nucleic acid base or analogue thereof, each of which is substituted as described in R¹, R^{1a} or R^{1b} for Formula (A), Formula (BB, Formula (C), Formula (D), Formula (A-1), Formula (B-1), Formula (C-1), Formula (D-1), Formula (E-1), or Formula (F-1).

### Embodiment 3

A compound of Formula (A-2), Formula (B-2), Formula (C-2), Formula (D-2), Formula (E-2) or Formula (F-2):

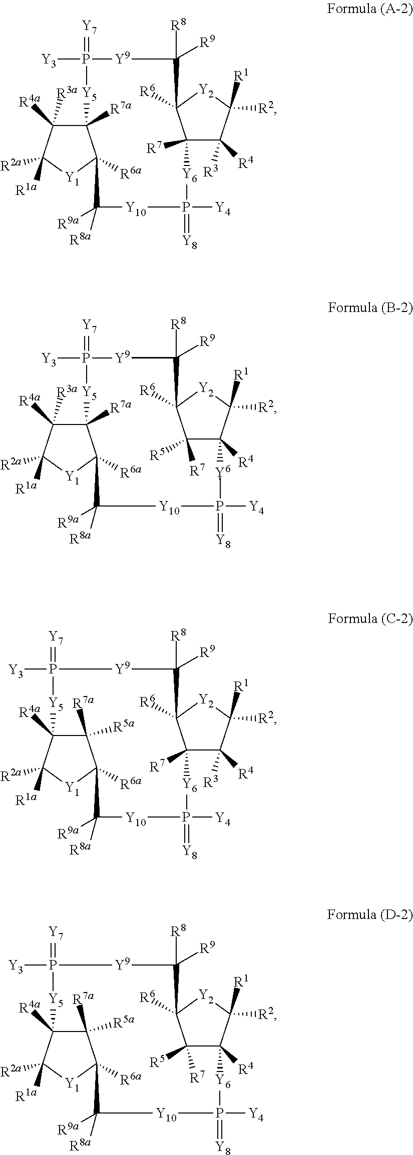

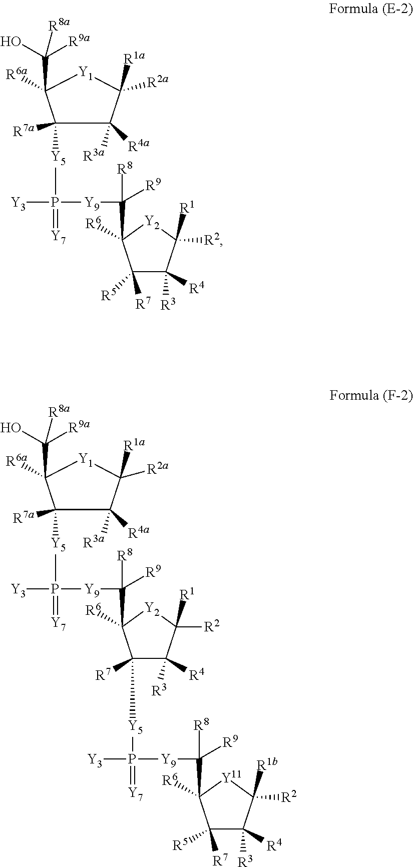

wherein R¹, R^{1a}, R^{1b}, R², R^{2a}, R³, R^{3a}, R⁴, R^{4a}, R⁵, R^{5a}, R⁶, R^{6a}, R⁷, R^{7a}, R⁸, R^{8a}, R⁹, Y₁, Y₂, Y₃, Y₄, Y₅, Y₆, Y₇, Y₈, Y₉, Y₁₀ and Y₁₁ are as defined above for compounds of Formula (A), Formula (B), Formula (C), Formula (D), Formula (E) and Formula (F).

### Embodiment 4

A compound of Formula (A), Formula (A-1) or Formula (A-2) of Embodiment 1, 2 or 3 wherein:
R² and R^{2a} are H;
one of R³ and R⁴ is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ or R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃;
R⁷ and R^{7a} are H;
R⁶ and R^{6a} are H;
R⁸, R⁹, R^{8a} and R^{9a} are independently H or C₁-C₆alkyl, and
one of R^{3a} and R^{4a} is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃.

### Embodiment 5

A compound of Formula (A), Formula (A-1) or Formula (A-2) of Embodiment 1, 2, 3 or 4 wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R⁶, R^{6a}, R⁷ and R^{7a} are H;
one of R^{3a} and R^{4a} is H and the other is H, OH or F;
one of R³ and R⁴ is H and the other is H, OH or F; and
R^{8a}, R^{9a}, R⁸ and R⁹ are independently selected from H or C₁-C₆alkyl.

### Embodiment 6

A compound of Formula (B), Formula (B-1) or Formula (B-2) of Embodiment 1, 2 or 3 wherein:
R² and R^{2a} are H;
one of R^{3a} and R^{4a} is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
R^{7a} and R^{6a} are H;
R⁶ and R⁴ are H;
R⁸, R⁹, R^{8a} and R^{9a} are independently H or C₁-C₆alkyl, and
one of R⁵ and R⁷ is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃.

### Embodiment 7

A compound of Formula (B), Formula (B-1) or Formula (B-2) of Embodiment 1, 2, 3 or 6 wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R^{7a}, R^{6a}, R⁶ and R⁴ are H;
one of R^{3a} and R^{4a} is H and the other is H, OH or F;
one of R⁵ and R⁷ is H and the other is H, OH or F, and
R^{8a}, R^{9a}, R⁸ and R⁹ are independently selected from H or C₁-C₆alkyl.

### Embodiment 8

A compound of Formula (C), Formula (C-1) or Formula (C-2) of Embodiment 1, 2 or 3 wherein:
R² and R^{2a} are H;
one of R³ and R⁴ is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃;
R^{4a} and R^{6a} are H;
R⁶ and R⁷ are H;
R⁸, R⁹, R^{8a} and R^{9a} are independently H or C₁-C₆alkyl;
one of R^{5a} and R^{7a} is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} or R^{7a} are substituted by 0, 1, 2 or 3 substituents independently selected from F, Cl, Br, I, OH, CN, and N₃.

### Embodiment 9

A compound of Formula (C), Formula (C-1) or Formula (C-2) of Embodiment 1, 2, 3 or 8 wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R^{4a}, R^{6a}, R⁶ and R⁷ are H;
one of R³ and R⁴ is H and the other is H, OH or F;
one of R^{5a} and R^{7a} is H and the other is H, OH or F, and
R^{8a}, R^{9a}, R⁸ and R⁹ are independently selected from H or C₁-C₆alkyl.

### Embodiment 10

A compound of Formula (D), Formula (D-1) or Formula (D-2) of Embodiment 1, 2 or 3 wherein:
R² and R^{2a} are H;
one of R^{5a} and R^{7a} is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} or R^{7a} are substituted by 0, 1, 2 or 3 substituents independently selected from F, Cl, Br, I, OH, CN, and N₃;
R^{4a} and R^{6a} are H;
R⁶ and R⁴ are H;
R⁸, R⁹, R^{8a} and R^{9a} are independently H or C₁-C₆alkyl, and
one of R⁵ and R⁷ is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃.

### Embodiment 11

A compound of Formula (D), Formula (D-1) or Formula (D-2) of Embodiment 1, 2, 3 or 10 wherein:
Y¹ and Y² are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y⁵ and Y⁶ are O or S;
Y₇ and Y₈ are O or S;
Y₉ and Y₁₀ are O or S;
R², R^{2a}, R^{4a}, R^{6a}, R⁶ and R⁴ are H;
one of R^{5a}, R^{7a} is H and the other is H, OH or F;
one of R⁵ and R⁷ is H and the other is H, OH or F, and
R⁸, R⁹, R^{8a} and R^{9a} are independently H or C₁-C₆alkyl.

### Embodiment 12

A compound of Formula (E), Formula (E-1) or Formula (E-2) of Embodiment 1, 2 or 3 wherein:
R² and R^{2a} are H;
R⁶ and R^{6a} are H;
R^{7a} is H;
R⁸, R⁹, R^{8a} and R^{9a} are independently H or C₁-C₆alkyl, and
one of R^{3a} and R^{4a} is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
one of R³ and R⁴ is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃, and
one of R⁵ and R⁷ is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃.

### Embodiment 13

A compound of Formula (E), Formula (E-1) or Formula (E-2) of Embodiment 1, 2, 3 or 12 wherein:
Y¹ and Y² are O, CH₂ or S;
Y₃ is OH, O⁻, OR¹⁰, N(R¹⁰)₂, SH or S⁻;
Y⁵ is O or S;
Y₇ is O or S;
Y₉ is O or S;
R², R^{2a}, R^{5a}, R^{6a}, R⁶ and R^{7a} are H;
one of R^{3a}, R^{4a} is H and the other is H, OH, OCH₃ or F;
one of R³, R⁴ is H and the other is H, OH, OCH₃ or F;
one of R⁵ and R⁷ is H and the other is H, OH, OCH₃ or F, and
R⁸, R⁹, R^{8a} and R^{9a} are independently H or C₁-C₆alkyl.

### Embodiment 14

A compound of Formula (F), Formula (F-1) or Formula (F-2) of Embodiment 1, 2 or 3 wherein:
R² and R^{2a} are H;
each R⁶ and R^{6a} are H;
each R^{7a} and R⁷ are H;
R⁸, R⁹, R^{8a} and R^{9a} are independently H or C₁-C₆alkyl, and
one of R^{3a} and R^{4a} is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
one of R³ and R⁴ is H and the other is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃, and
R⁵ is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ is substituted by 0, 1, 2 or 3 substituents independently selected from F, Cl, Br, I, OH, CN, and N₃.

### Embodiment 15

A compound of Formula (F), Formula (F-1) or Formula (F-2) of Embodiment 1, 2, 3 or 12 wherein:
Y¹ and Y² are O, CH₂ or S;
each Y₃ is OH, O⁻, OR¹⁰, N(R¹⁰)₂, SH or S⁻;
each Y⁵ is O or S;
each Y₇ is independently O or S;
each Y₉ is independently O or S;
Y¹¹ is O, CH₂ or S;
R², R^{2a}, R⁶, R^{6a}, R⁶, R⁷ and R^{7a} are H;
one of R^{3a}, R^{4a} is H and the other is H, OH, OCH₃ or F;
one of R³, R⁴ is H and the other is H, OH, OCH₃ or F;
R⁵ is H, OH, OCH₃ or F, and
R⁸, R⁹, R^{8a} and R^{9a} are independently H or C₁-C₆alkyl.

### Embodiment 16

A compound of any one of Embodiments 1 to 15 wherein:
R¹ is

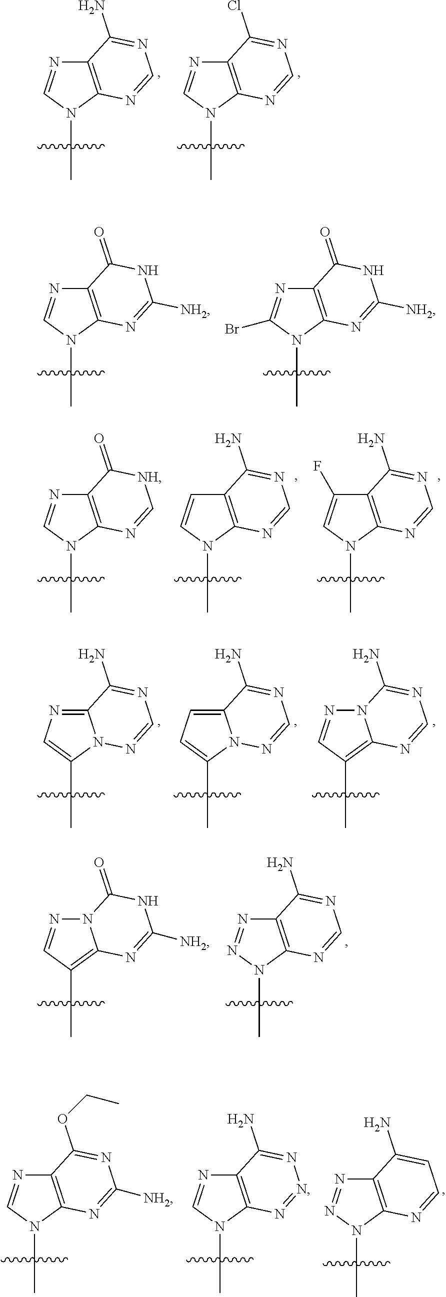

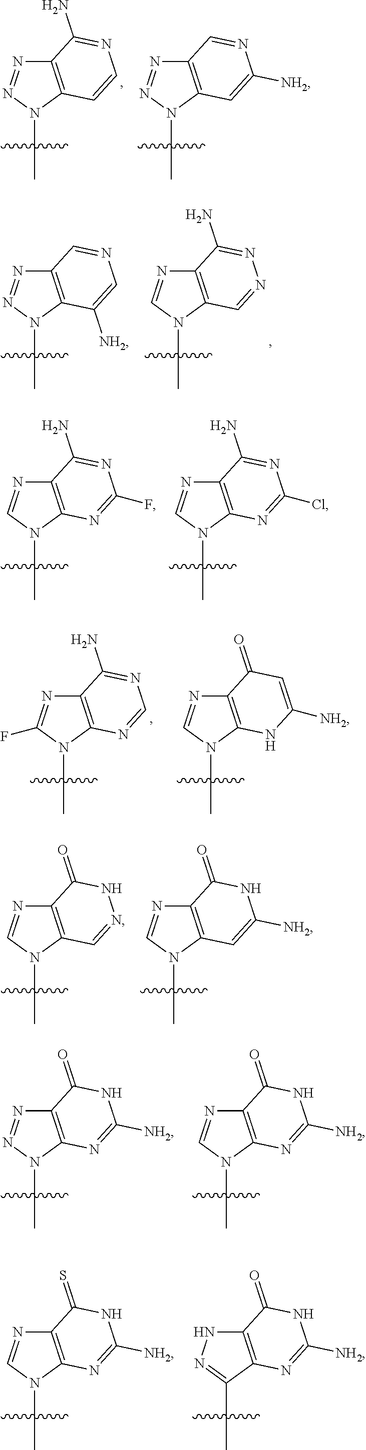

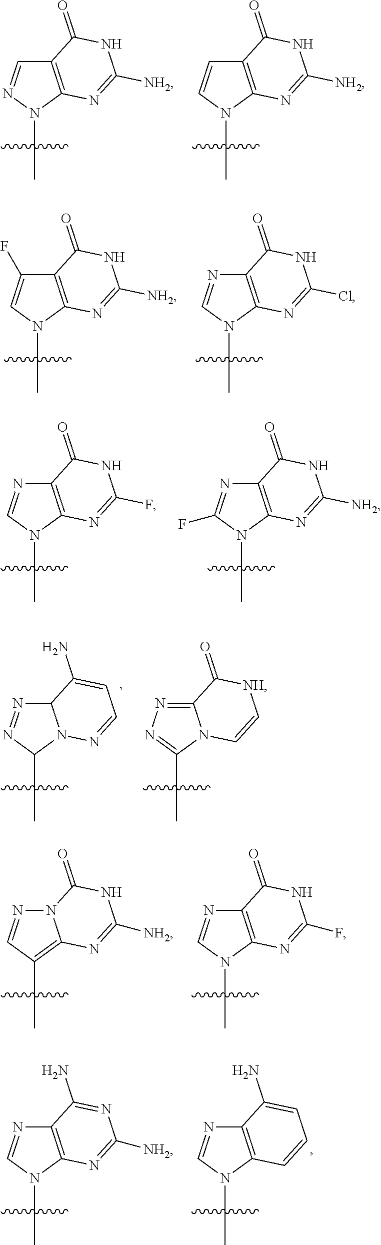

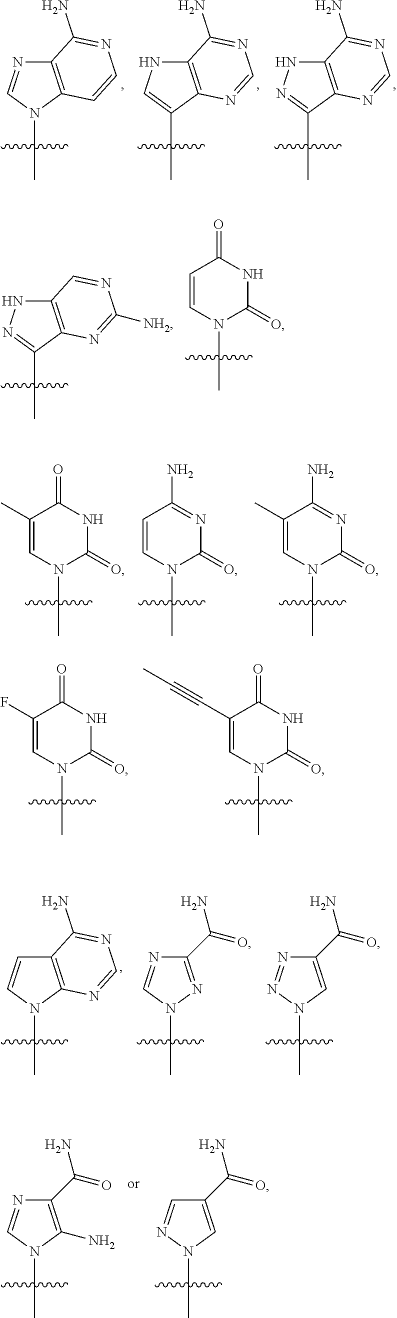

wherein R¹ is substituted with 0, 1 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1a} is

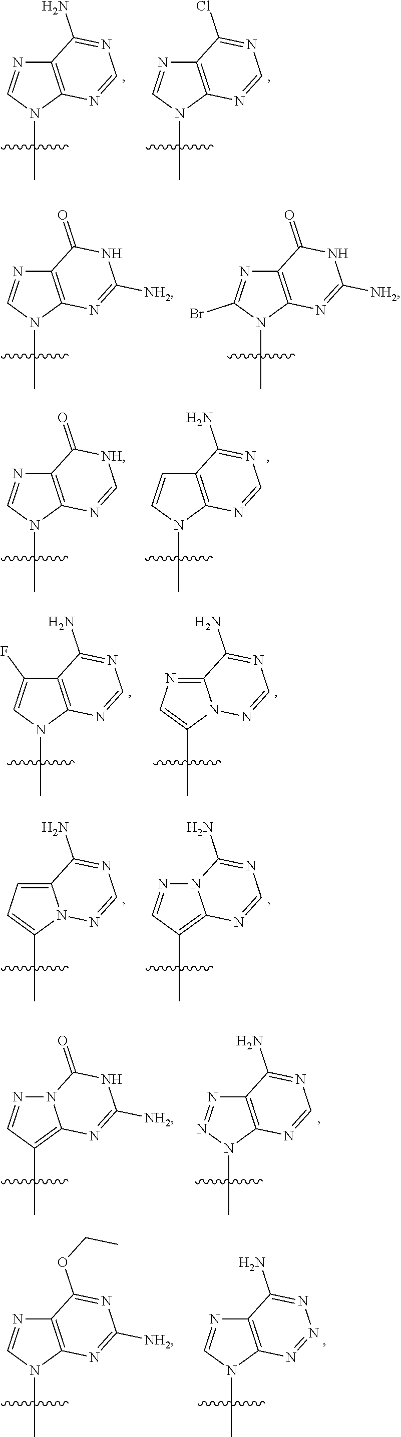

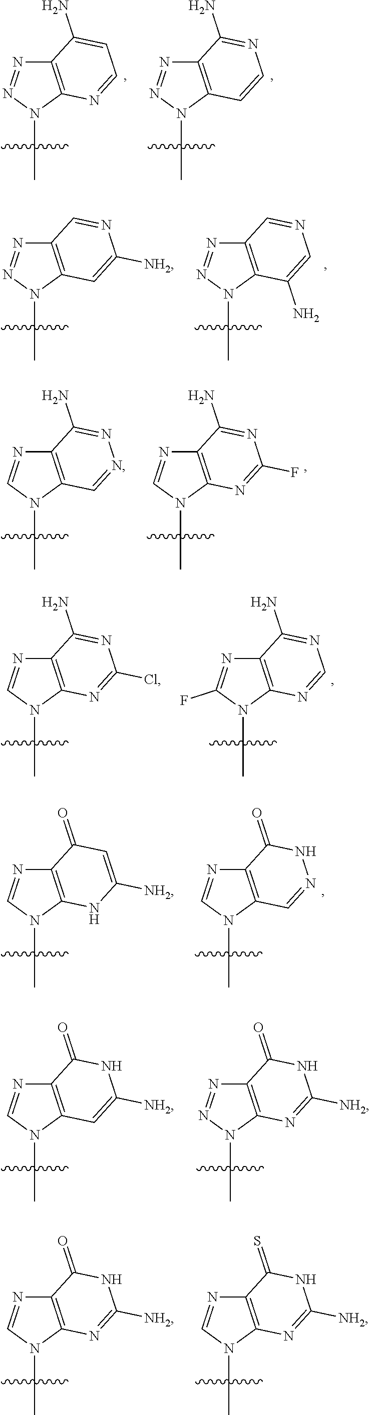

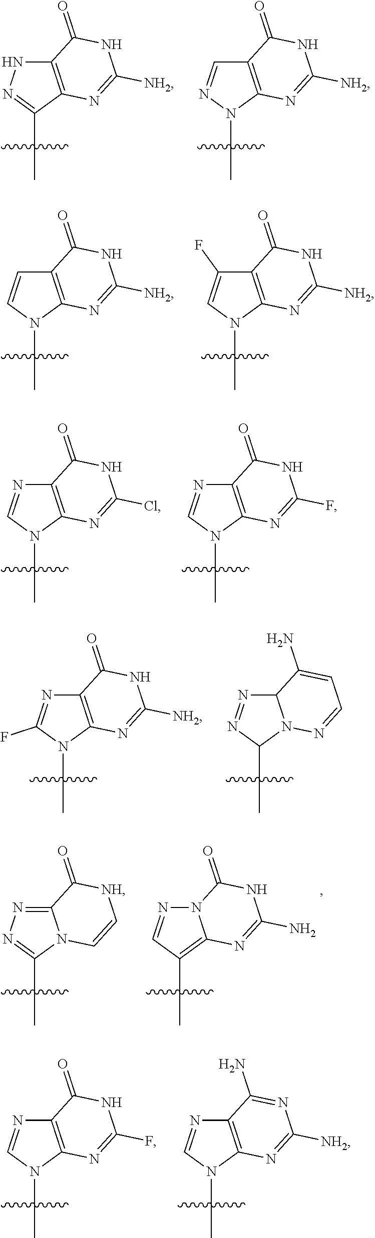

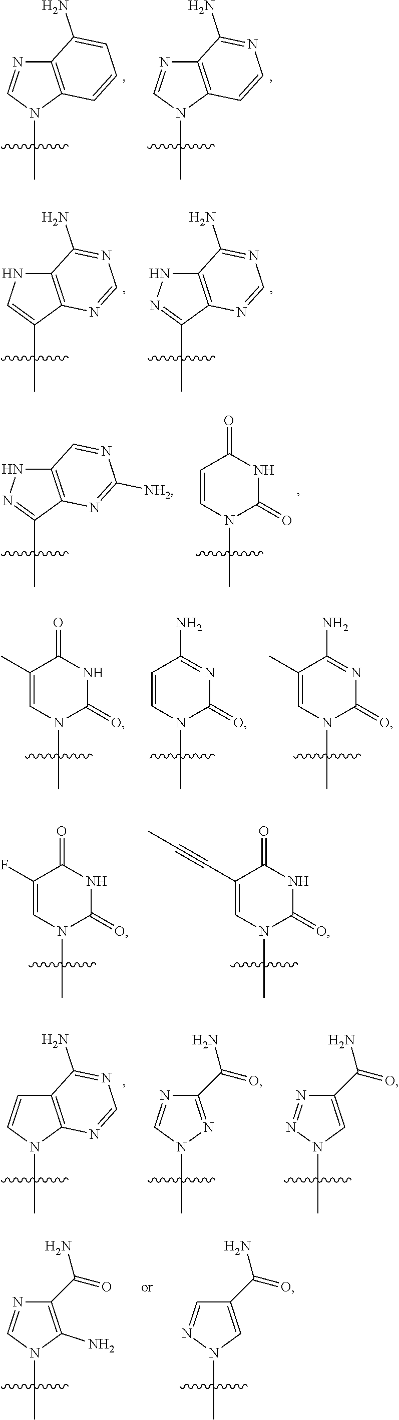

wherein: R^{1a} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
and
R^{1b} is

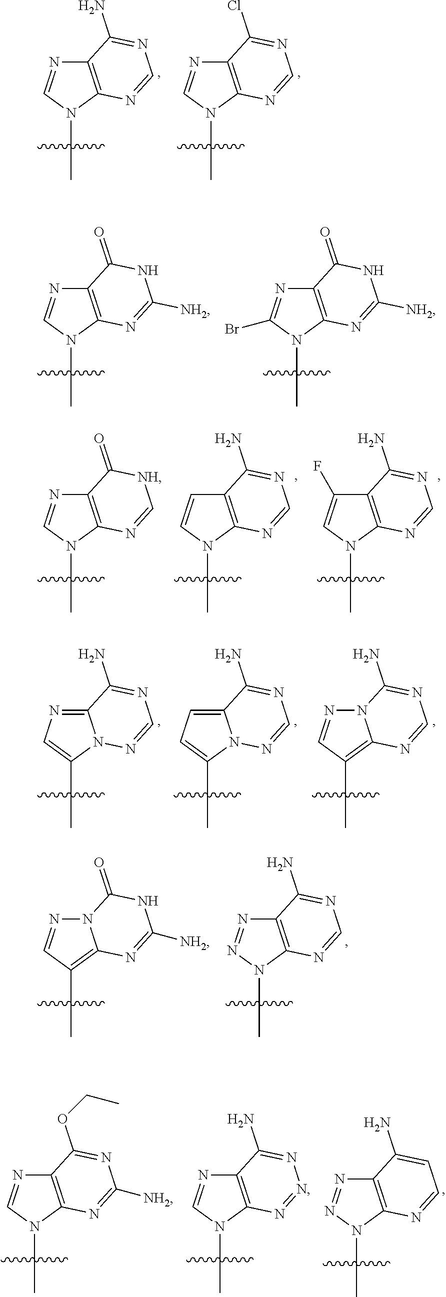

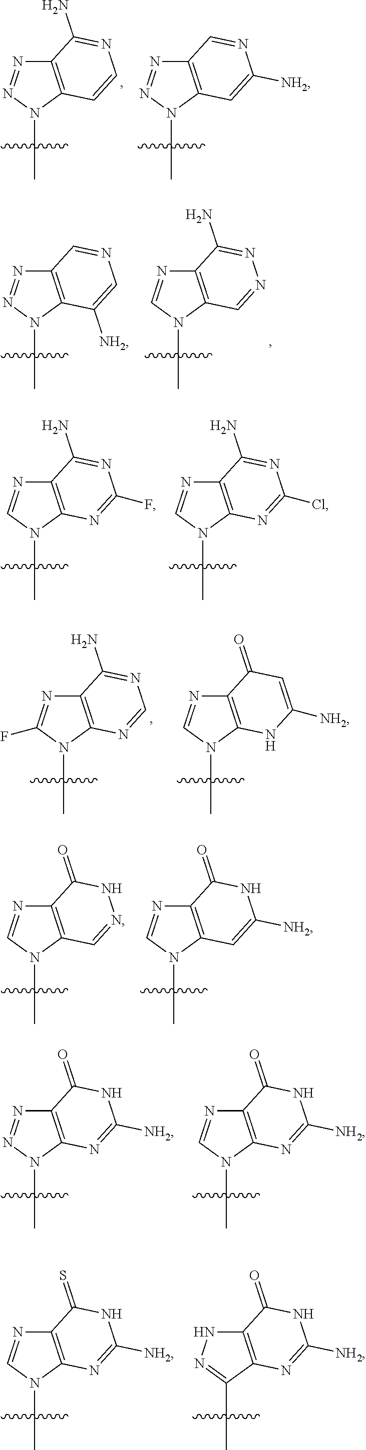

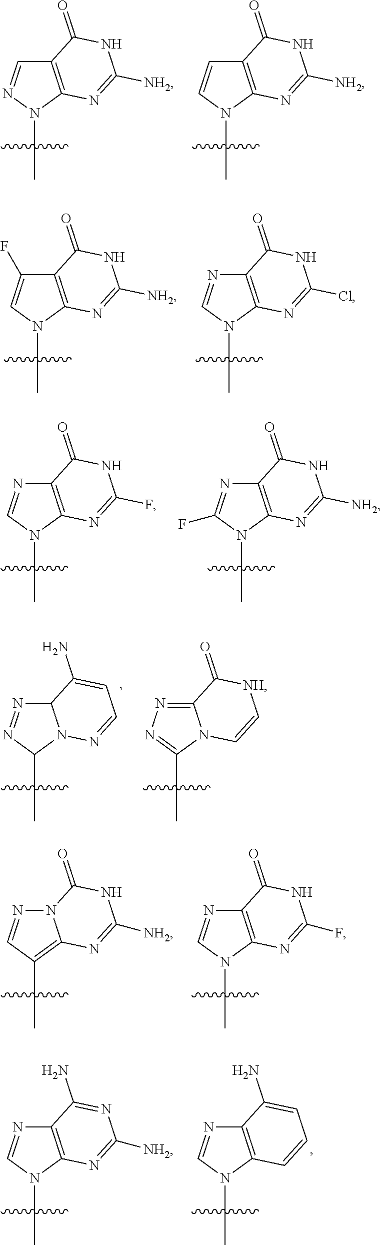

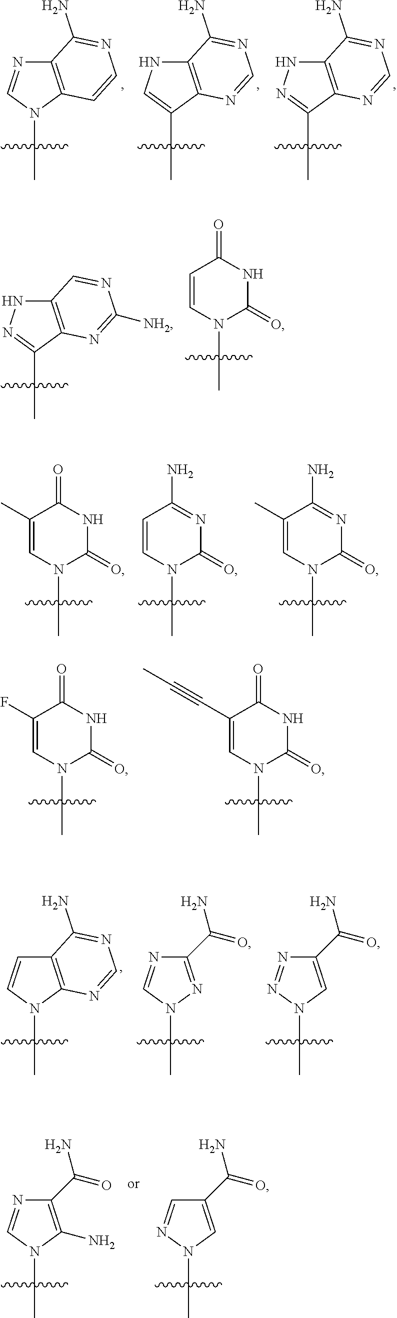

wherein R^{1b} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂.

### Embodiment 17

A compound of Formula (A-3), Formula (B-3), Formula (C-3), Formula (D-3), Formula (E-3) or Formula (F-3):

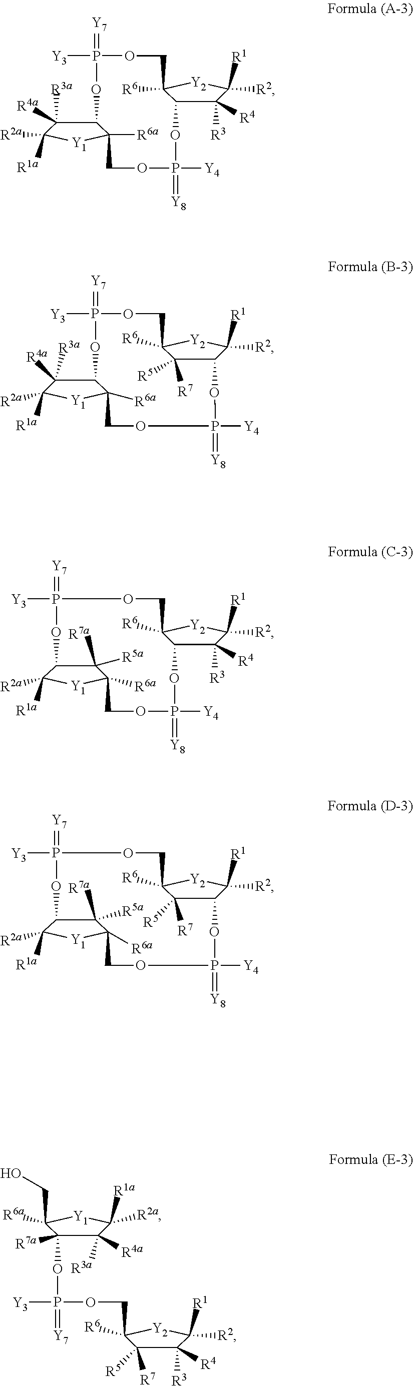

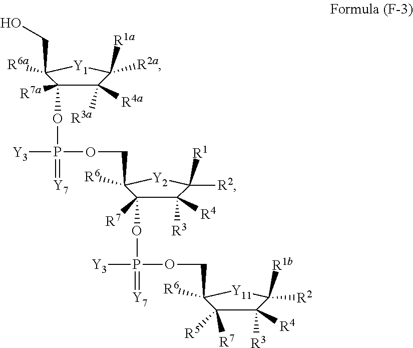

wherein:
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₁₁ is —O—, —S—, —S(═O)—, —SO₂—, —CH₂—, or —CF₂—;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₇ is O or S;
Y₈ is O or S;
R¹ is

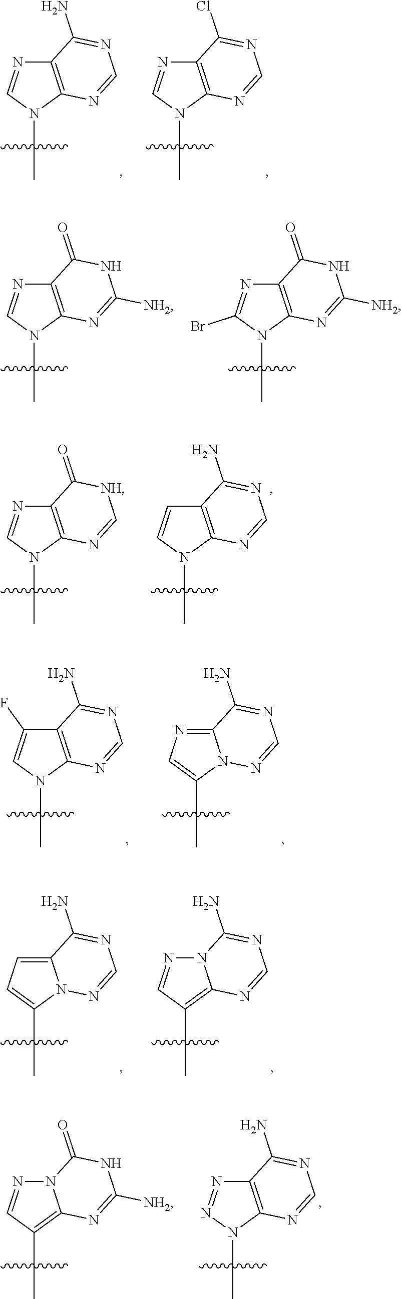

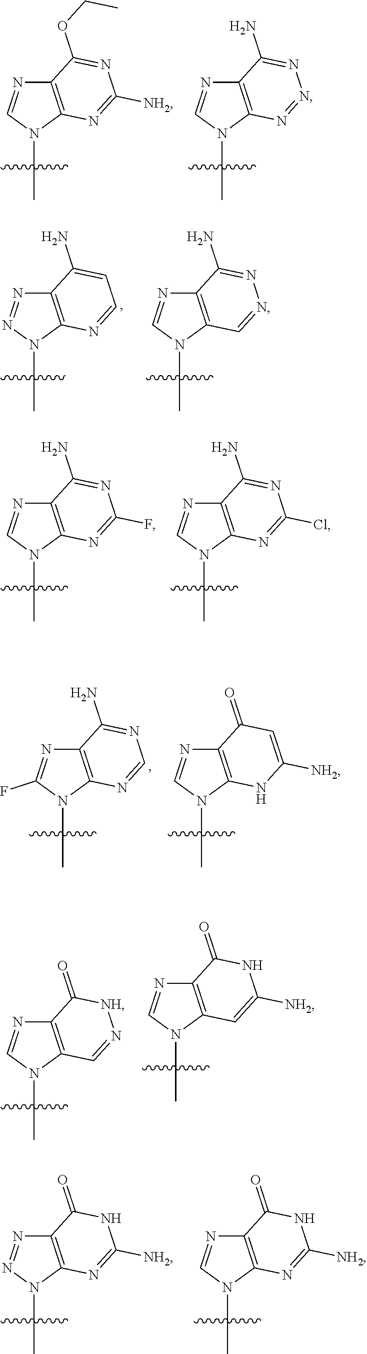

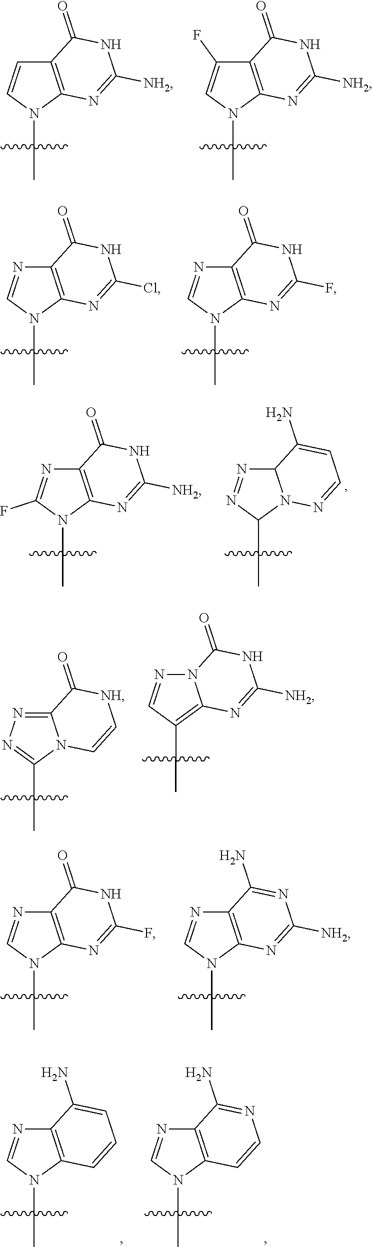

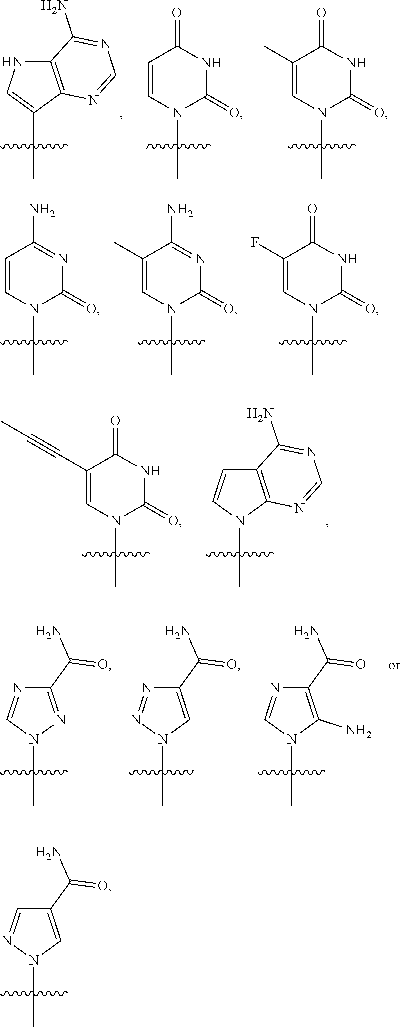

wherein R¹ is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1a} is

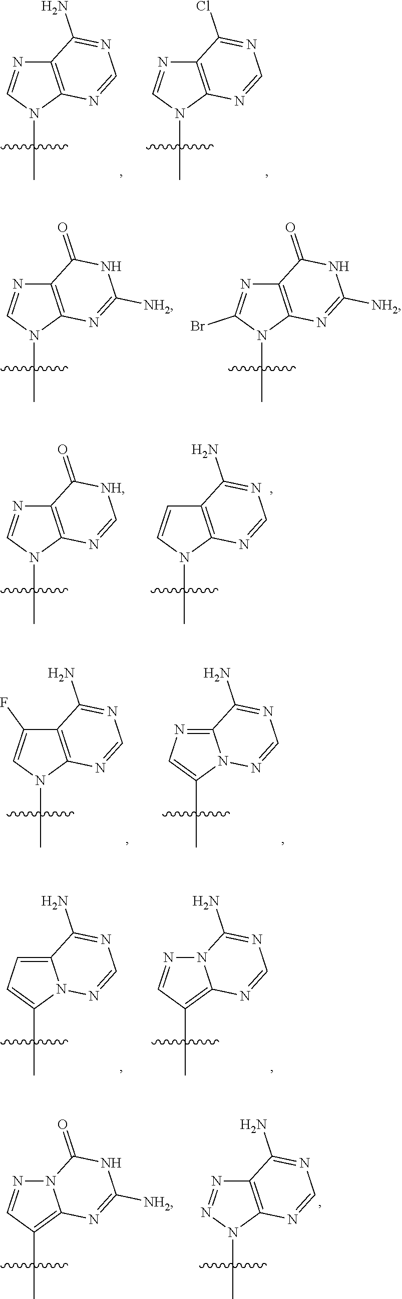

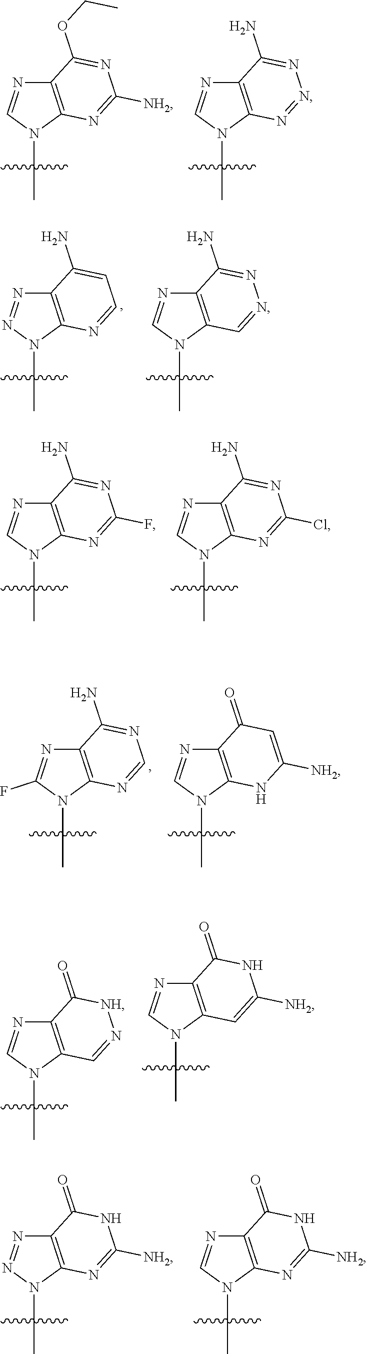

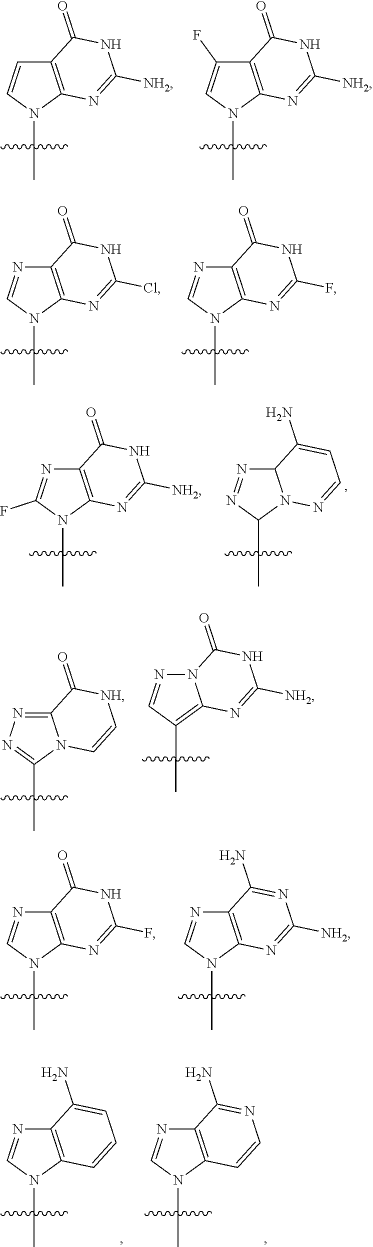

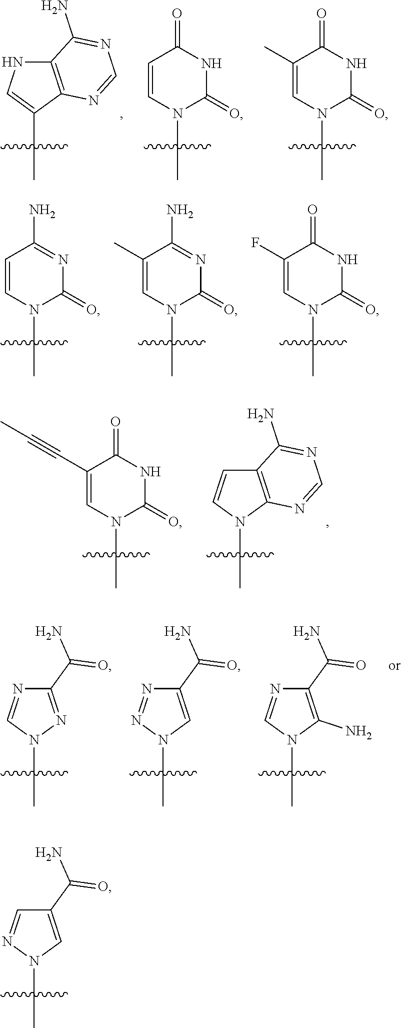

wherein: R^{1a} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
and
R^{1b} is

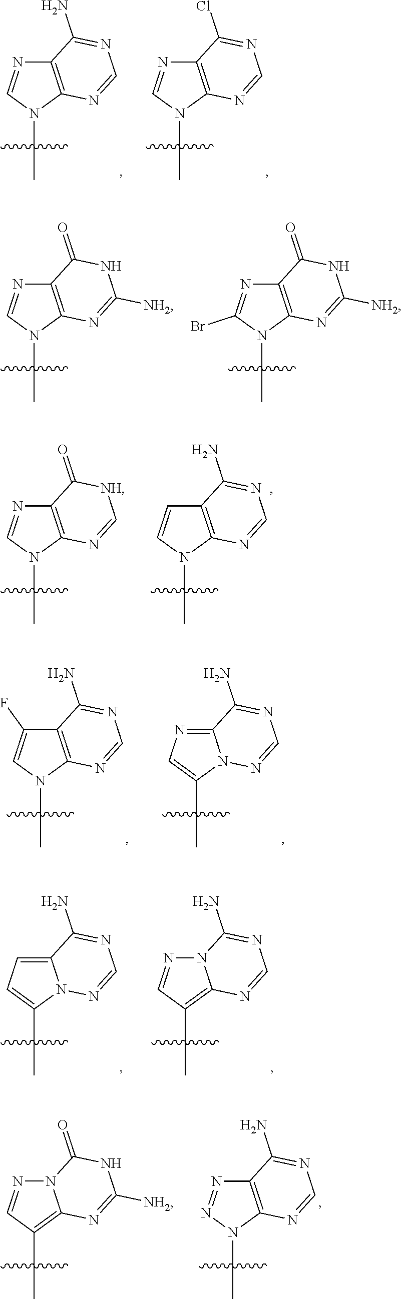

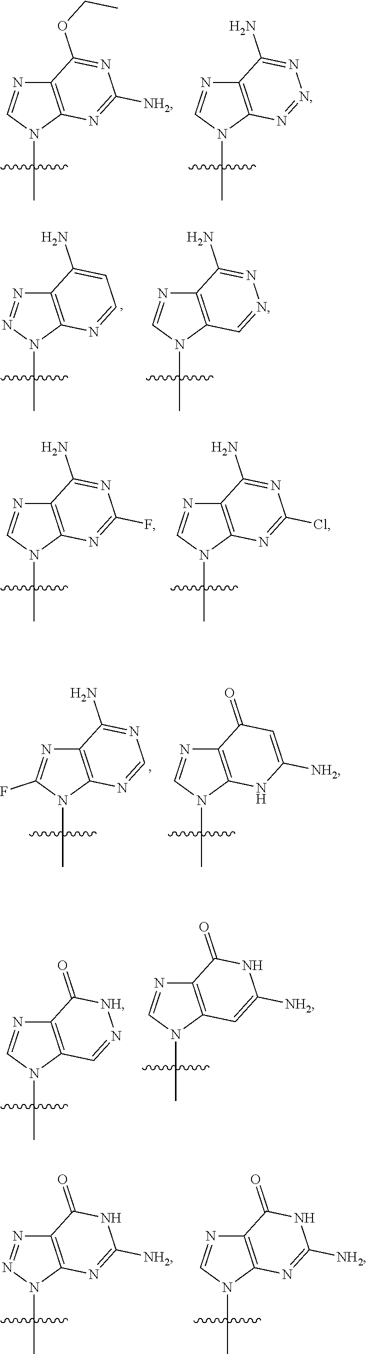

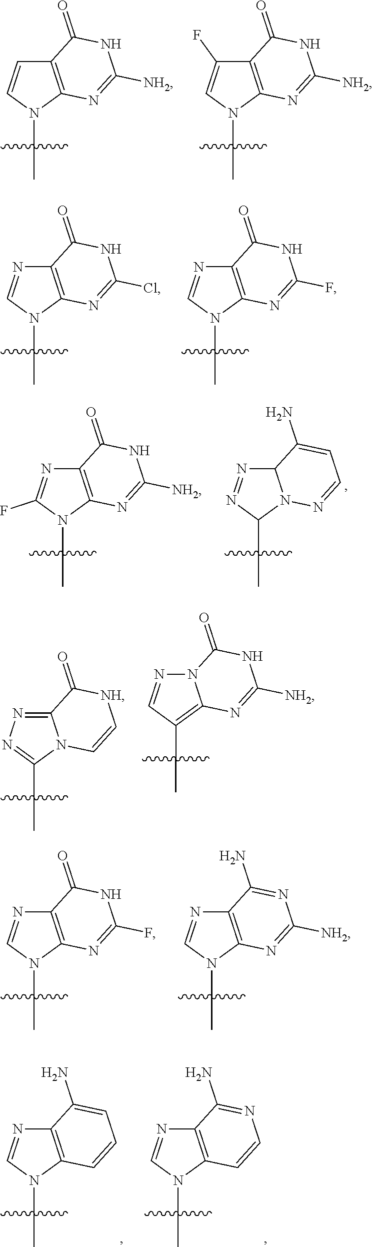

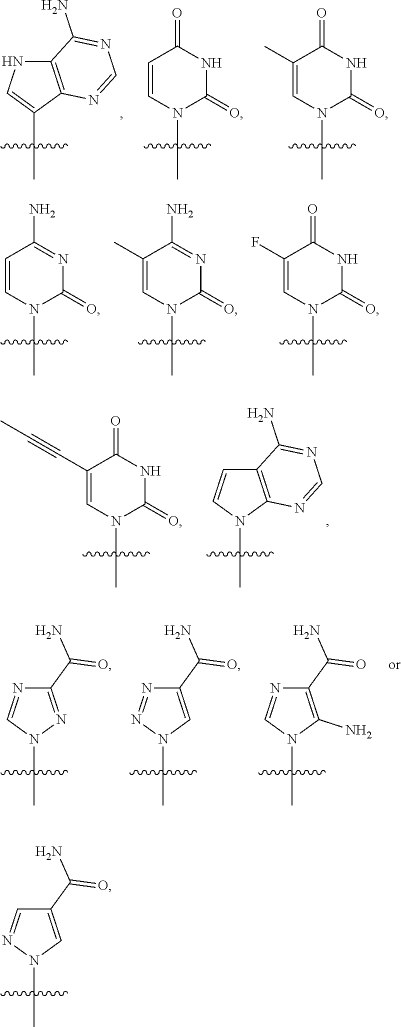

wherein R^{1b} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
   each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
R^{3a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
R^{4a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
R^{5a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
R^{7a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
each R¹⁰ is independently selected from the group consisting of H, C₁-C₁₂alkyl, —(CH₂CH₂O)ₙCH₂CH₂C(═O)OC₁-C₆alkyl, and

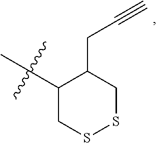 [SMILES: *C1CSSCC1CC#C]

wherein the C₁-C₁₂alkyl of R¹⁰ is substituted by 0, 1, 2 or 3 substituents independently selected from —OH, C₁-C₁₂alkoxy, —S—C(═O)C₁-C₆alkyl and C(O)OC₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position, and
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position.

### Embodiment 18

The compound Formula (A-3), or a pharmaceutically acceptable salt thereof, having the structure of Formula (A-4), or a pharmaceutically acceptable salt thereof:

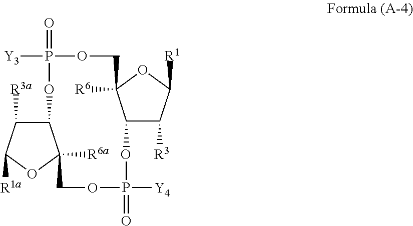

wherein: R¹, R^{1a}, R³, R^{3a}, R⁶, R^{6a}, Y₃ and Y₄ are as defined in Embodiment 17.

### Embodiment 19

The compound of Formula (A-4), or a pharmaceutically acceptable salt thereof, having the structure of Formula (A-4a), Formula A-4b), Formula A-4c) or Formula A-4d), or a pharmaceutically acceptable salt thereof:

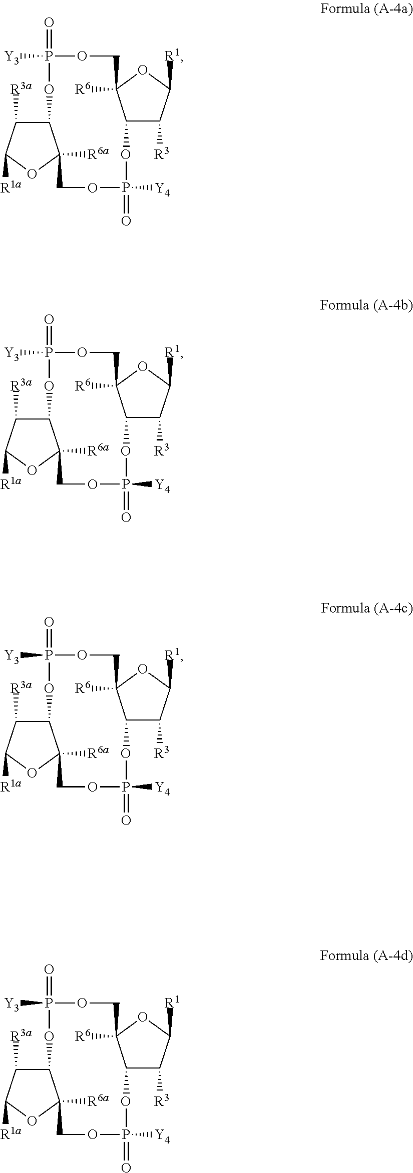

wherein: R¹, R^{1a}, R³, R^{3a}, R⁶ and R^{6a} are as defined in Embodiment 17;
Y₃ is OR¹⁰, N(R¹⁰)₂, SH or S⁻, and
Y₄ is OR¹⁰, N(R¹⁰)₂, SH or S⁻.

### Embodiment 20

The compound of Formula (A-4), or a pharmaceutically acceptable salt thereof, having the structure of Formula (A-4e), Formula (A-4f), Formula (A-4 g), Formula (A-4h), Formula (A-4i), Formula (A-4j), Formula (A-4k), Formula (A-41), Formula (A-4m), Formula (A-4n), Formula (A-4o) or Formula (A-4p), or a pharmaceutically acceptable salt thereof:

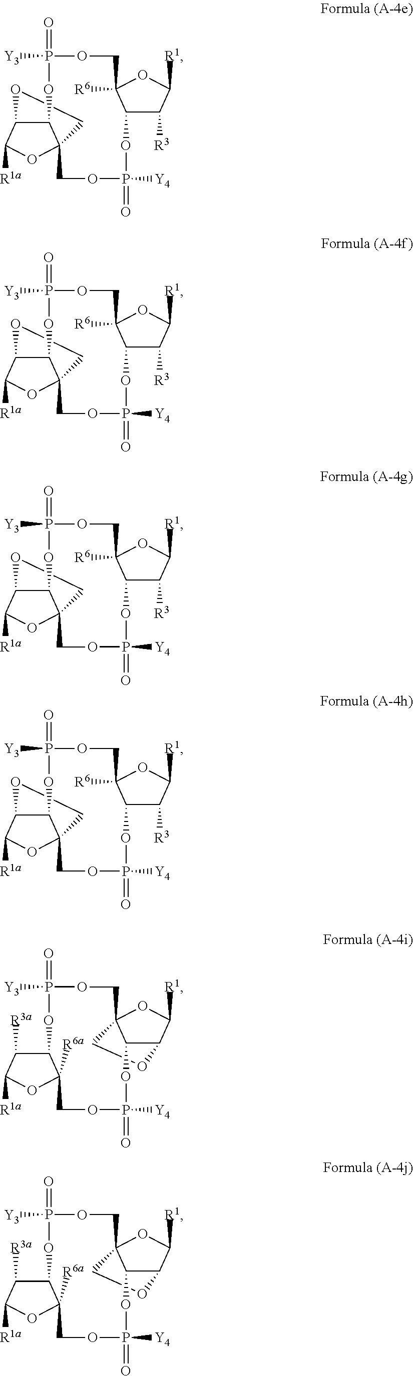

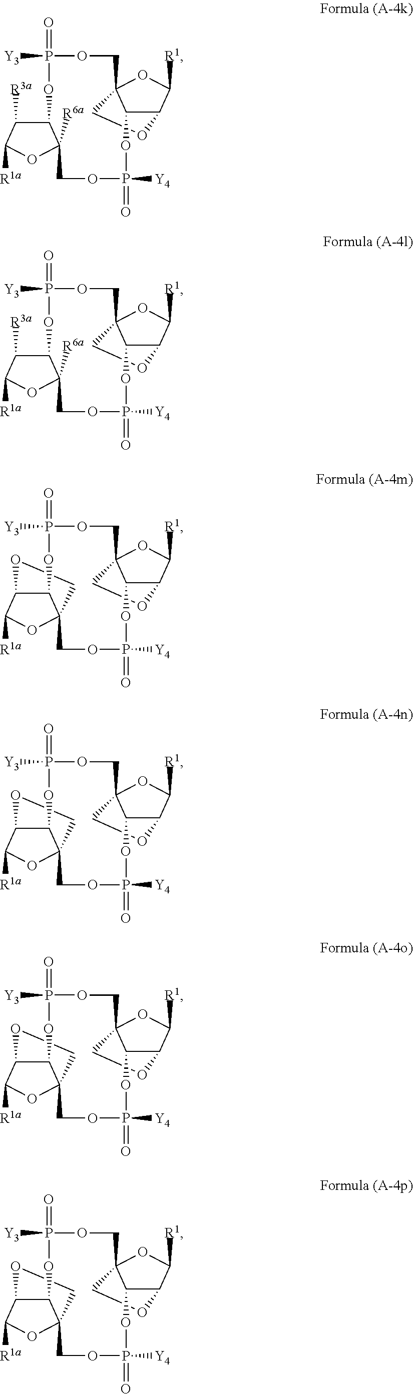

wherein: R¹, R^{1a}, R³, R^{3a}, R⁶ and R^{6a} are as defined in Embodiment 17;
Y₃ is OR¹⁰, N(R¹⁰)₂, SH or S⁻, and
Y₄ is OR¹⁰, N(R¹⁰)₂, SH or S⁻.

### Embodiment 21

The compound of Formula (B-3) having the structure of Formula (B-4), or a pharmaceutically acceptable salt thereof:

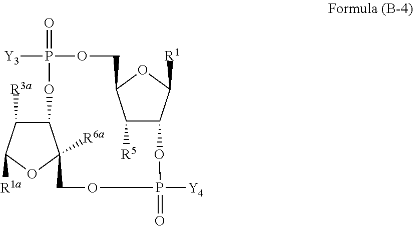

wherein: R¹, R^{1a}, R³, R^{3a}, R⁵, R^{6a}, Y₃ and Y₄ are as defined in Embodiment 17.

### Embodiment 22

The compound of Formula (B-4), or a pharmaceutically acceptable salt thereof, having the structure of Formula (B-4a), Formula (B-4b), Formula (B-4c) or Formula (B-4d), or a pharmaceutically acceptable salt thereof:

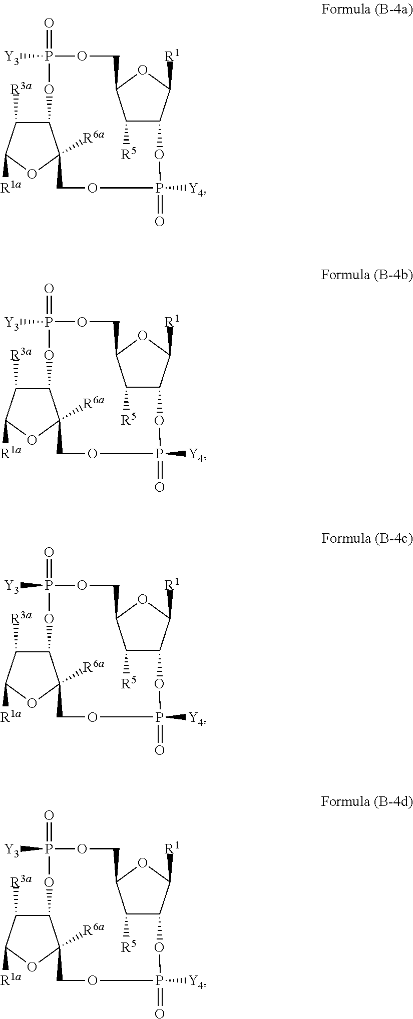

wherein: R¹, R^{1a}, R^{3a}, R⁵ and R^{6a} are as defined in Embodiment 13;
Y₃ is OR¹⁰, N(R¹⁰)₂, SH or S⁻, and
Y₄ is OR¹⁰, N(R¹⁰)₂, SH or S⁻.

### Embodiment 23

The compound of Formula (B-4), or a pharmaceutically acceptable salt thereof, having the structure of Formula (B-4e), Formula (B-4f), Formula (B-4 g) or Formula (B-4h), or a pharmaceutically acceptable salt thereof:

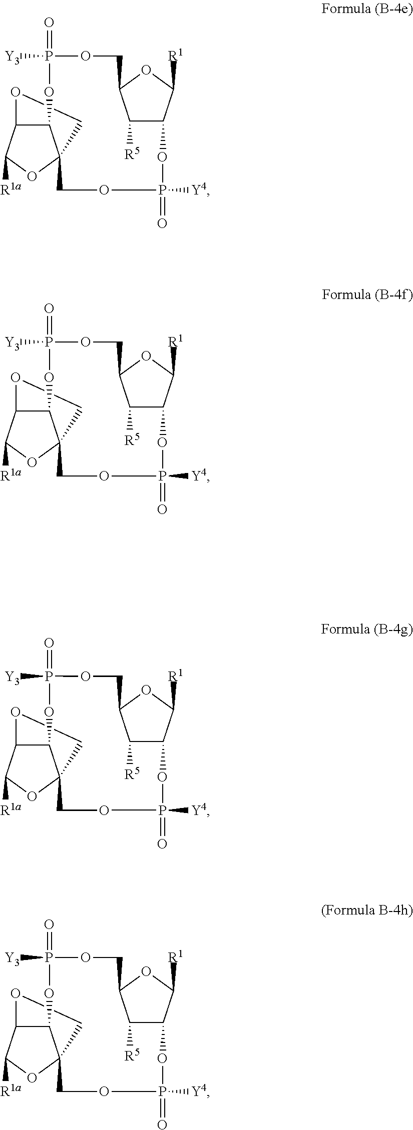

wherein: R¹, R^{1a} and R⁵ are as defined in Embodiment 17;
Y₃ is OR¹⁰, N(R¹⁰)₂, SH or S⁻, and
Y₄ is OR¹⁰, N(R¹⁰)₂, SH or S⁻.

### Embodiment 24

The compound of Formula (C-3) having the structure of Formula (C-4), or a pharmaceutically acceptable salt thereof:

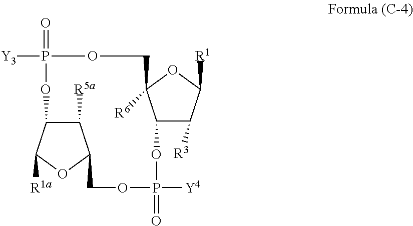

wherein: R¹, R^{1a}, R³, R^{5a}, R⁶, R^{6a}, Y₃ and Y₄ are as defined in Embodiment 17.

### Embodiment 25

The compound of Formula (C-4), or a pharmaceutically acceptable salt thereof, having the structure of Formula (C-4a), Formula (C-4b), Formula (C-4c) or Formula (C-4d), or a pharmaceutically acceptable salt thereof:

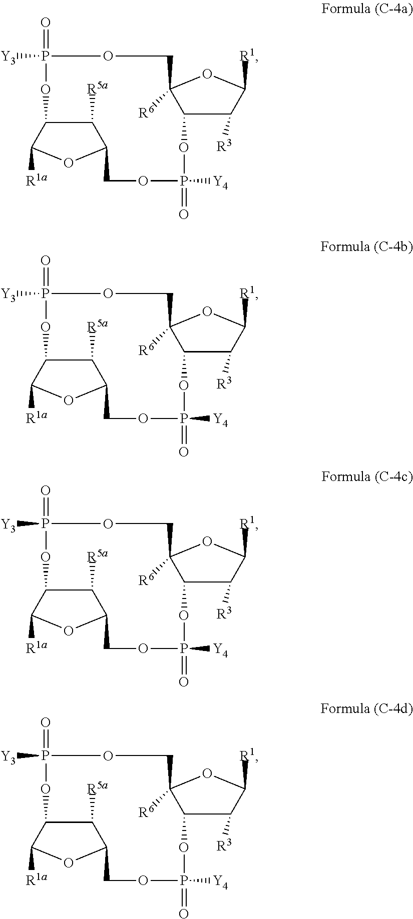

wherein: R¹, R^{1a}, R³, R^{5a} and R⁶ are as defined in Embodiment 17; Y₃ is OR¹⁰, N(R¹⁰)₂, SH or S⁻, and Y₄ is OR¹⁰, N(R¹⁰)₂, SH or S⁻.

### Embodiment 26

The compound of Formula (C-4), or a pharmaceutically acceptable salt thereof, having the structure of Formula (C-4e), Formula (C-4f), Formula (C-4 g) or Formula (C-4h), or a pharmaceutically acceptable salt thereof:

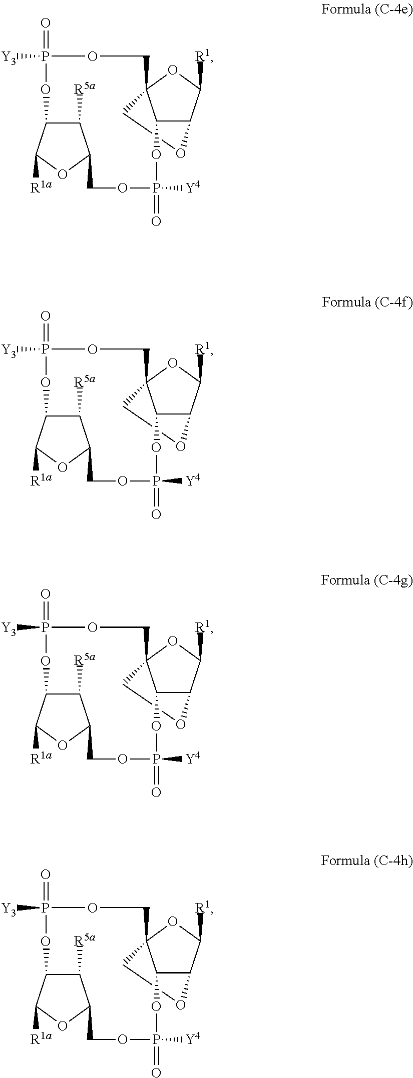

wherein: R¹, R^{1a} and R^{5a} are as defined in Embodiment 17;
Y₃ is OR¹⁰, N(R¹⁰)₂, SH or S⁻, and
Y₄ is OR¹⁰, N(R¹⁰)₂, SH or S⁻.

### Embodiment 27

The compound of Formula (D-3), or a pharmaceutically acceptable salt thereof, having the structure of Formula (D-4), or a pharmaceutically acceptable salt thereof:

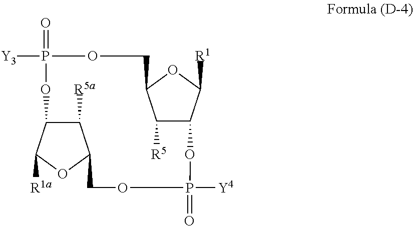

wherein: R¹, R^{1a}, R⁵, R^{5a}, Y₃ and Y₄ are as defined in Embodiment 17.

### Embodiment 28

The compound of Formula (D-4), or a pharmaceutically acceptable salt thereof, having the structure of Formula (D-4a), Formula (D-4b), Formula (D-4c) or Formula (D-4d), or a pharmaceutically acceptable salt thereof:

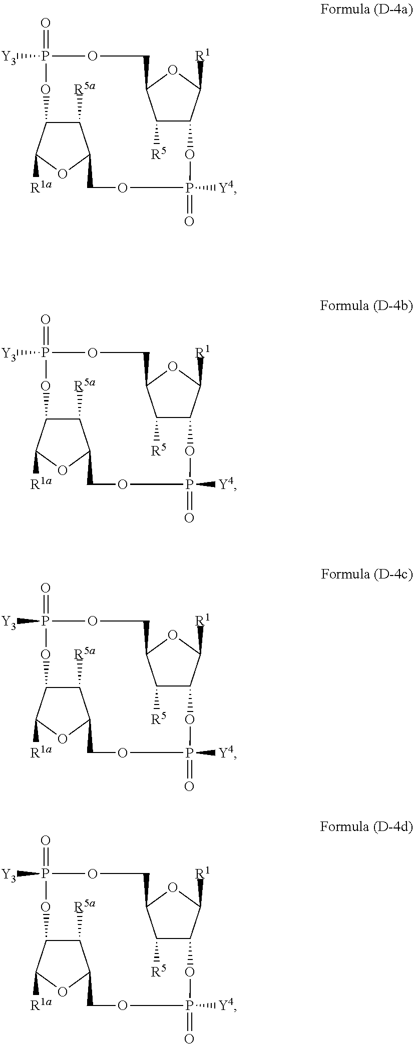

wherein: R¹, R^{1a}, R⁵ and R^{5a} are as defined in Embodiment 17;
Y₃ is OR¹⁰, N(R¹⁰)₂, SH or S⁻, and
Y₄ is OR¹⁰, N(R¹⁰)₂, SH or S⁻.

### Embodiment 29

The compound of Formula (E-3), or a pharmaceutically acceptable salt thereof, having the structure of Formula (E-4), or a pharmaceutically acceptable salt thereof:

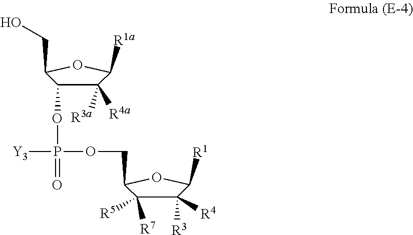

wherein: R¹, R^{1a}, R³, R^{3a}, R⁴, R^{4a}, R⁵ and R⁷ are as defined in Embodiment 17.

### Embodiment 30

The compound of Formula (E-4), or a pharmaceutically acceptable salt thereof, having the structure of Formula (E-4a) or Formula (E-4b), or a pharmaceutically acceptable salt thereof:

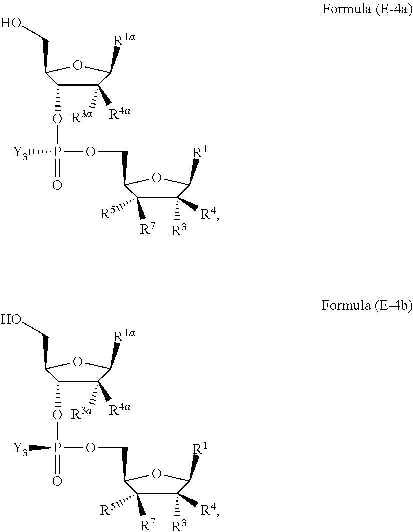

wherein: R¹, R^{1a}, R³, R^{3a}, R⁴, R^{4a}, R⁵ and R⁷ are as defined in Embodiment 17;
and
Y₃ is OR¹⁰, N(R¹⁰)₂, SH or S⁻.

### Embodiment 31

The compound of Formula (F-3), or a pharmaceutically acceptable salt thereof, having the structure of Formula (F-4), or a pharmaceutically acceptable salt thereof:

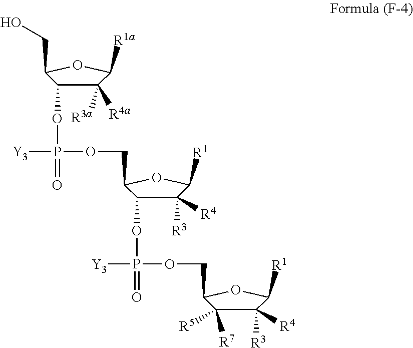

wherein: R¹, R^{1a}, R^{1b}, R³, R^{3a}, R⁴, R^{4a}, R⁵ and R⁷ are as defined in Embodiment 17.

### Embodiment 32

The compound of Formula (F-4), or a pharmaceutically acceptable salt thereof, having the structure of Formula (F-4a), Formula (F-4b), Formula (F-4c), or Formula (F-4d), or a pharmaceutically acceptable salt thereof:

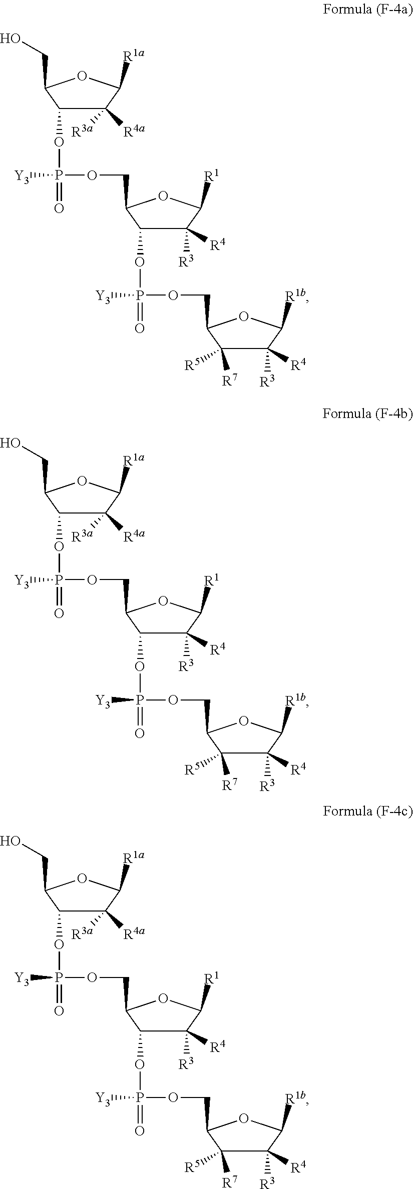

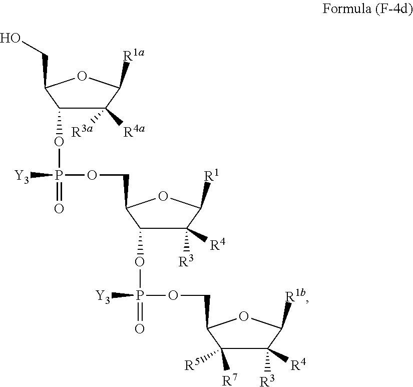

wherein: R¹, R^{1a}, R^{1b}, R³, R^{3a}, R⁴, R^{4a}, R⁵ and R⁷ are as defined in Embodiment 17;
and
each Y₃ is independently selected from OR¹⁰, N(R¹⁰)₂, SH and S⁻.

### Embodiment 33

The compound of any one of Embodiments 1 to 32, wherein R¹ is

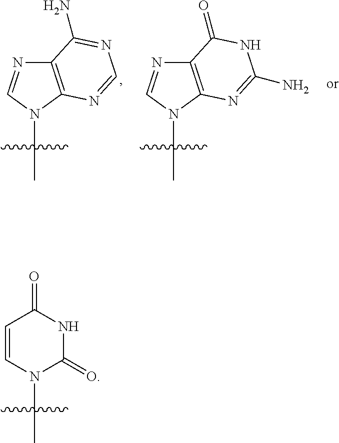

### Embodiment 34

The compound of any one of Embodiments 1 to 32, wherein R^{1a} is

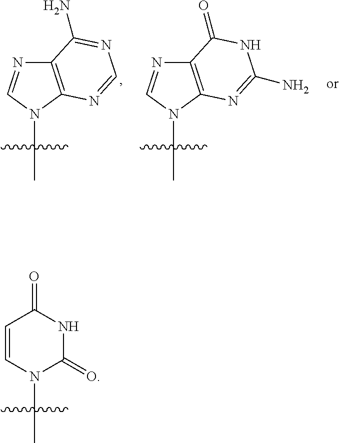

### Embodiment 35

The compound of any one of Embodiments 1 to 32, wherein R^{1b} is

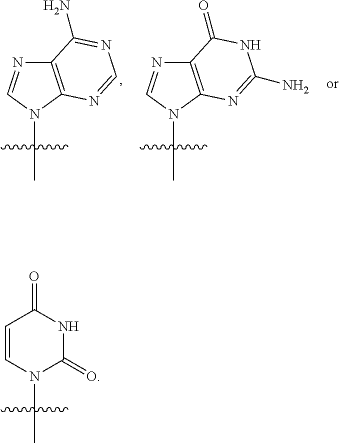

### Embodiment 36

The compound of any one of Embodiments 1 to 32, wherein R¹ is

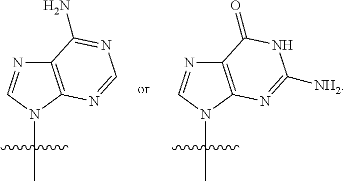 [SMILES: *n1cnc2c(N)ncnc21 ; "or" ; *n1cnc2c(=O)[nH]c(N)nc21]

### Embodiment 37

The compound of any one of Embodiments 1 to 32, wherein R^{1a} is

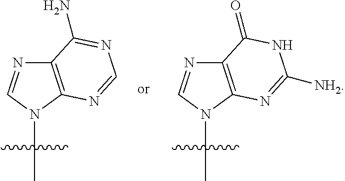 [SMILES: *n1cnc2c(N)ncnc21 ; "or" ; *n1cnc2c(=O)[nH]c(N)nc21]

### Embodiment 38

The compound of any one of Embodiments 1 to 32, wherein R^{1b} is

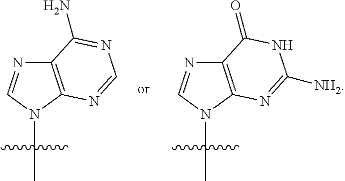 [SMILES: *n1cnc2c(N)ncnc21 ; "or" ; *n1cnc2c(=O)[nH]c(N)nc21]

### Embodiment 39. The compound of any one of Embodiments 1 to 32, wherein R¹ is

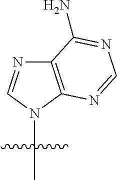 [SMILES: *n1cnc2c(N)ncnc21]

### and R^{1a} is

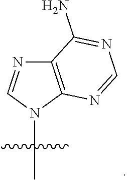 [SMILES: *n1cnc2c(N)ncnc21]

### Embodiment 40

The compound of any one of Embodiments 1 to 32 wherein R¹ is

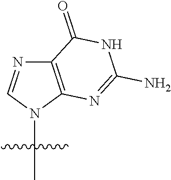 [SMILES: *n1cnc2c(=O)[nH]c(N)nc21]

### and R^{1a} is

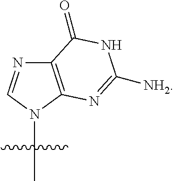 [SMILES: *n1cnc2c(=O)[nH]c(N)nc21]

### Embodiment 41

The compound of any one of Embodiments 1 to 32, wherein R¹ is

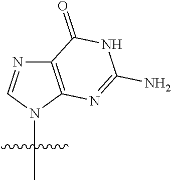 [SMILES: *n1cnc2c(=O)[nH]c(N)nc21]

### and R^{1a} is

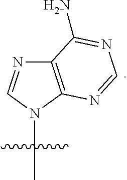 [SMILES: *n1cnc2c(N)ncnc21]

### Embodiment 42

The compound of any one of Embodiments 1 to 32, wherein R¹ is

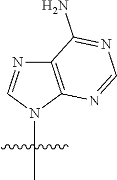 [SMILES: *n1cnc2c(N)ncnc21]

### and R^{1a} is

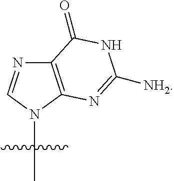 [SMILES: *n1cnc2c(=O)[nH]c(N)nc21]

### Embodiment 43

The compound of any one of Embodiments 1 to 32, wherein R¹ is

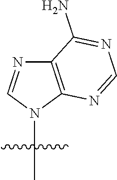 [SMILES: *n1cnc2c(N)ncnc21]

### and R^{1a} is

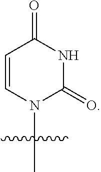 [SMILES: *n1ccc(=O)[nH]c1=O]

### Embodiment 44

The compound of any one of Embodiments 1 to 32, wherein R¹ is

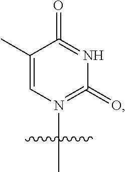

### R^{1b} is

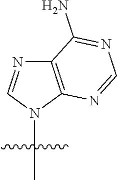 [SMILES: *n1cnc2c(N)ncnc21]

### and R^{1a} is

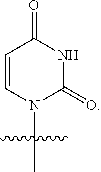 [SMILES: *n1ccc(=O)[nH]c1=O]

### Embodiment 45

The compound of any one of Embodiments 1 to 44, wherein:
Y₃ is OH, O⁻, SH or S⁻, and
Y₄ is OH, O⁻, SH or S⁻.

### Embodiment 46

The compound of any one of Embodiments 1 to 44, wherein:
Y₃ is OH or O⁻, and
Y₄ is OH or O⁻.

### Embodiment 47

The compound of any one of Embodiments 1 to 44, wherein:
Y₃ is SH or S⁻, and
Y₄ is OH or O⁻.

### Embodiment 48

The compound of any one of Embodiments 1 to 44, wherein:
Y₃ is OH or O⁻, and
Y₄ is SH or S⁻

### Embodiment 49

The compound of any one of Embodiments 1 to 44, wherein:
Y₃ is SH or S⁻, and
Y₄ is SH or S⁻

### Embodiment 50

The compound of any one of Embodiments 1 to 49 wherein:
R³ is —OH or F;
R^{3a} is —OH or F;
R⁵ is —OH or F;
R^{5a} is —OH or F;
R⁶ is H, and
R^{6a} is H.

### Embodiment 51

The compound of any one of Embodiments 1 to 49 wherein:
R³ is H, —OH or F;
R^{3a} is H, —OCH₃, —OH or F;
R⁵ is —OH or F;
R⁴, R^{4a}, R⁶, R^{6a}, R⁷, R^{7a} are H, and
R^{6a} is H.

### Embodiment 52

A Drug moiety (D) is a compound of Table 1:

TABLE 1

| Compound | |
|---|---|
| No. | Structure |
| T1-1 | 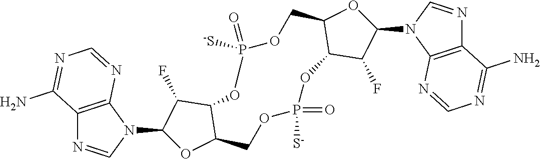 |
| T1-2 | 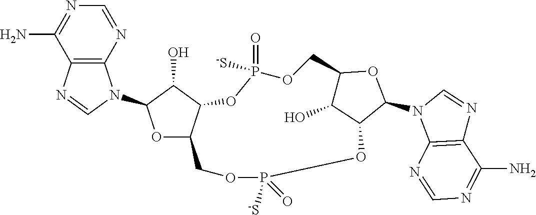 |
| T1-3 | 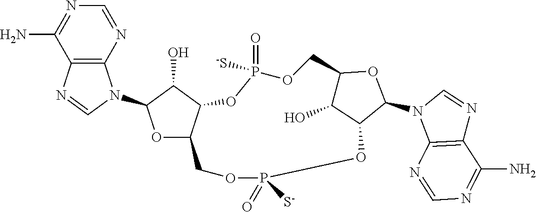 |
| T1-4 | 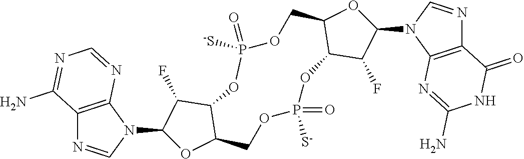 |
| T1-5 | 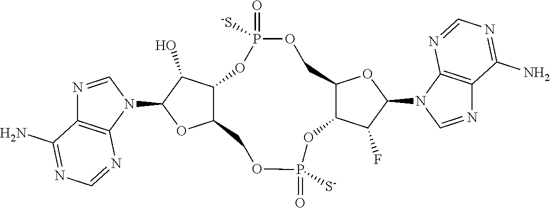 |
| T1-6 | 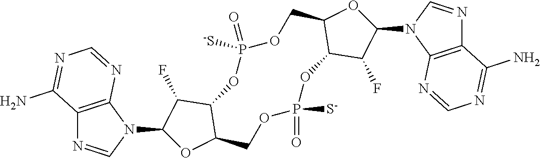 |
| T1-7 | 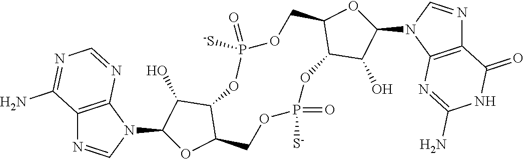 |
| T1-8 | 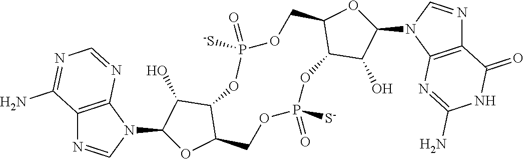 |
| T1-9 | 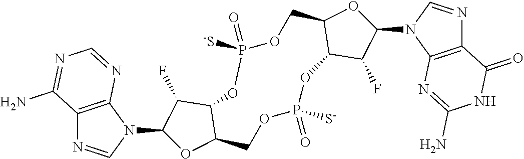 |
| T1-10 | 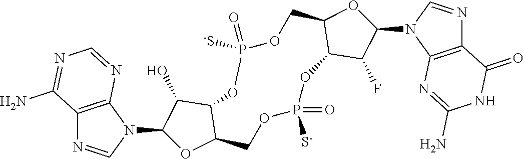 |
| T1-11 | 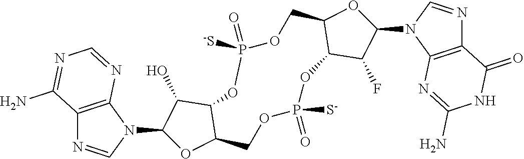 |
| T1-12 | 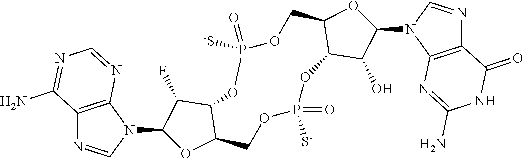 |
| T1-13 | 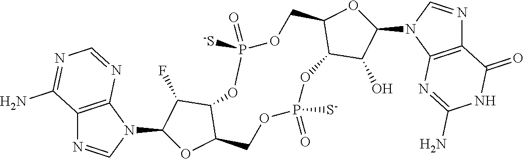 |
| T1-14 | 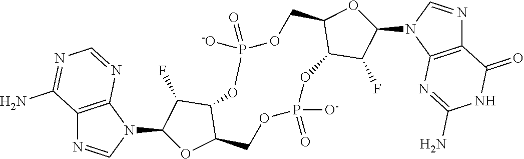 |
| T1-15 | 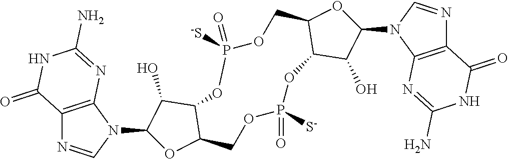 |
| T1-16 | 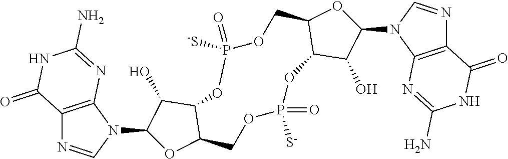 |
| T1-17 | 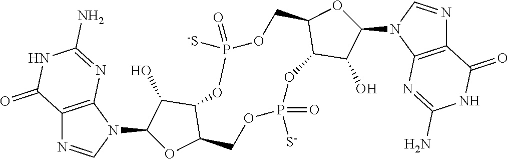 |
| T1-18 | 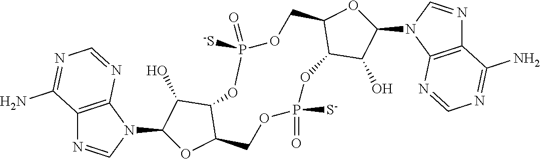 |
| T1-19 | 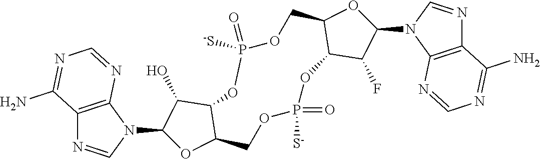 |
| T1-20 | 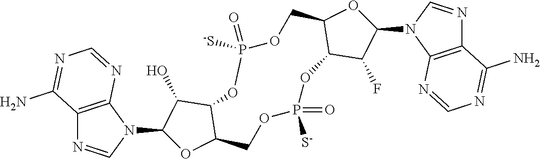 |
| T1-21 | 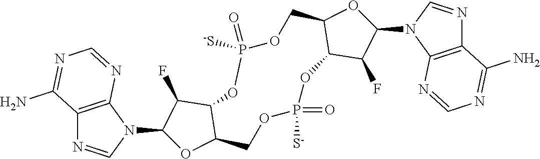 |
| T1-22 | 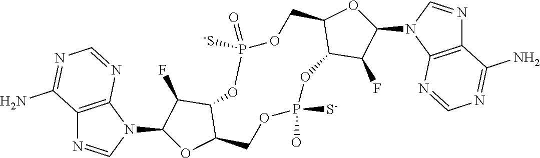 |
| T1-23 | 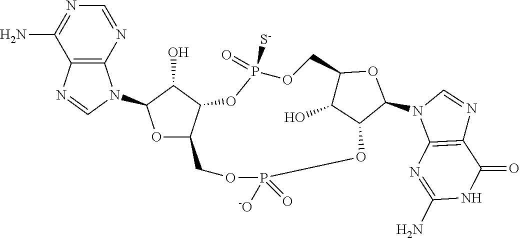 |
| T1-24 | 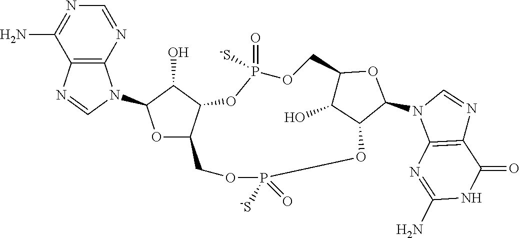 |
| T1-25 | 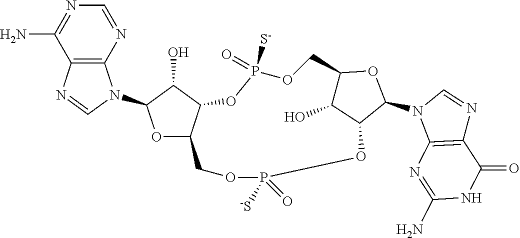 |
| T1-26 | 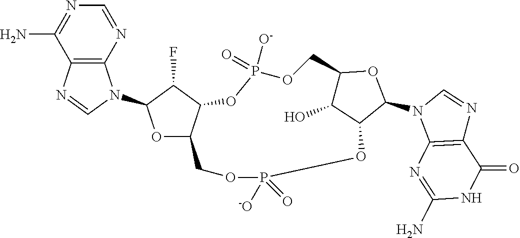 |
| T1-27 | 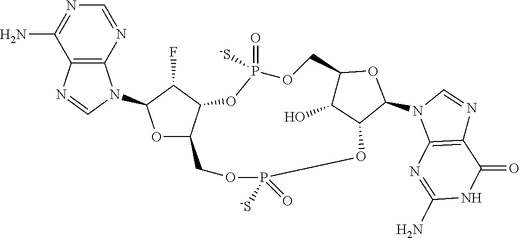 |
| T1-28 | 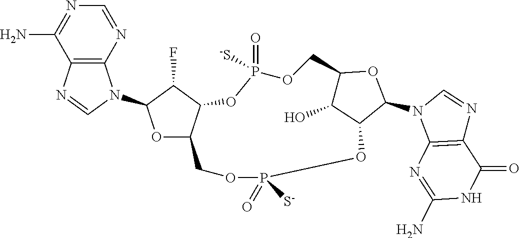 |
| T1-29 | 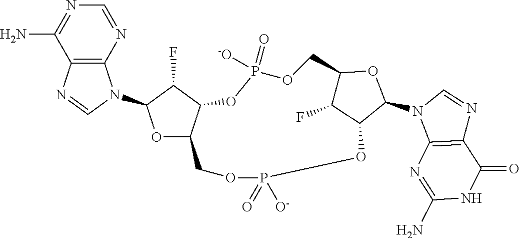 |
| T1-30 | 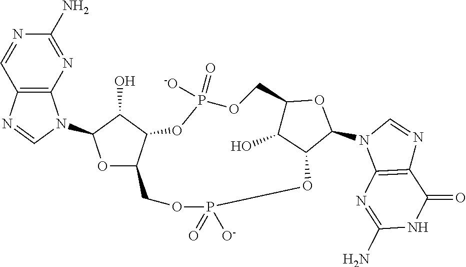 |
| T1-31 | 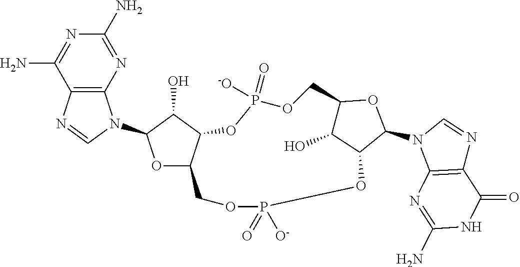 |
| T1-32 | 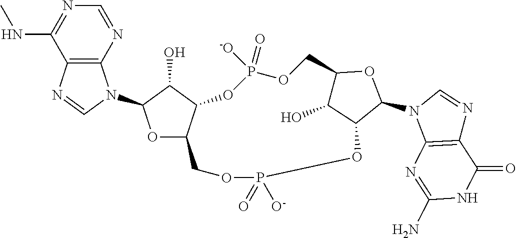 |
| T1-33 | 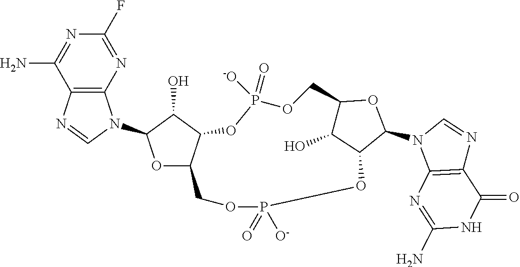 |
| T1-34 | 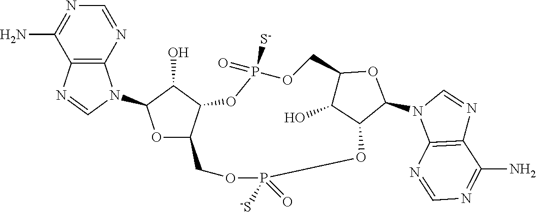 |
| T1-35 | 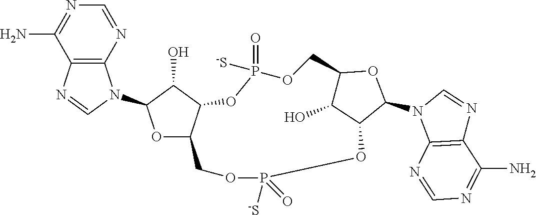 |
| T1-36 | 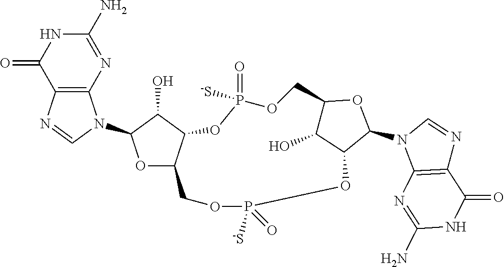 |
| T1-37 | 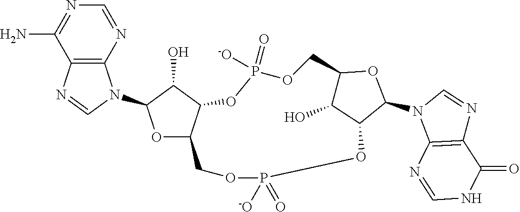 |
| T1-38 | 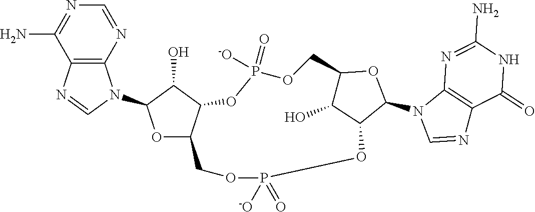 |
| T1-39 | 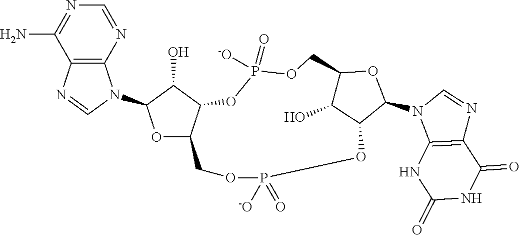 |
| T1-40 | 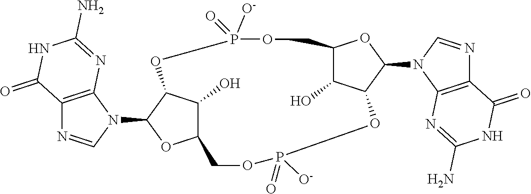 [SMILES: Nc1nc2c(ncn2[C@@H]2O[C@@H]3COP(=O)([O-])O[C@@H]4[C@H](O)[C@@H](COP(=O)([O-])O[C@@H]2[C@@H]3O)O[C@H]4n2cnc3c(=O)[nH]c(N)nc32)c(=O)[nH]1] |
| T1-41 | 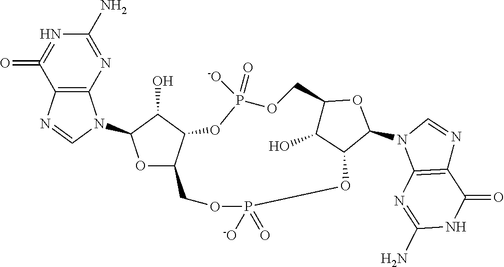 |
| T1-42 | 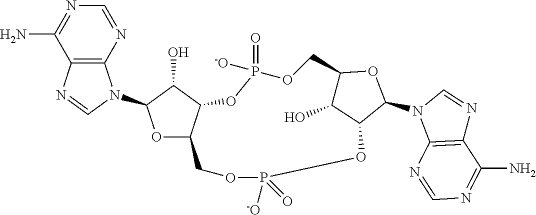 |
| T1-43 | 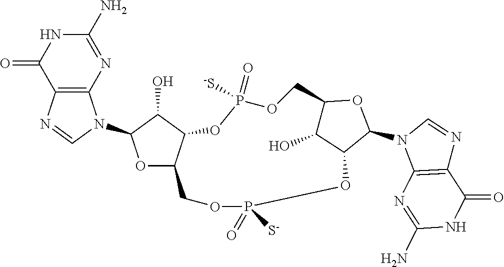 |
| T1-44 | 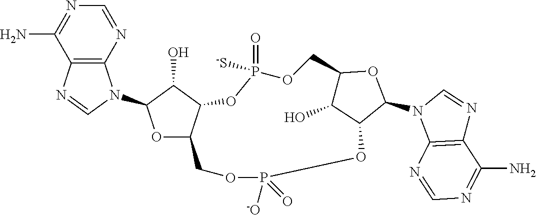 |
| T1-45 | 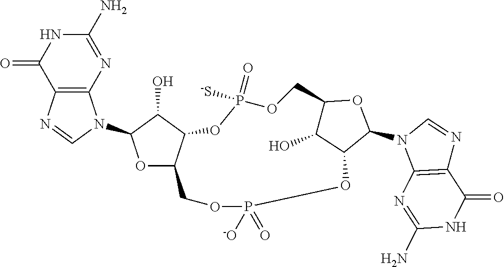 |
| T1-46 | 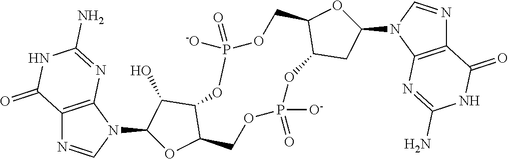 |
| T1-47 | 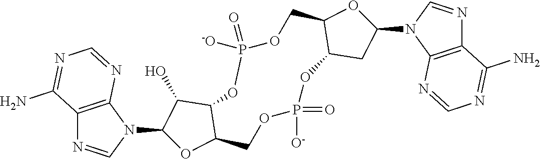 |
| T1-48 | 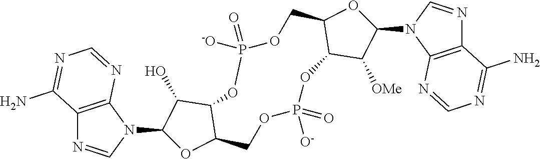 |
| T1-49 | 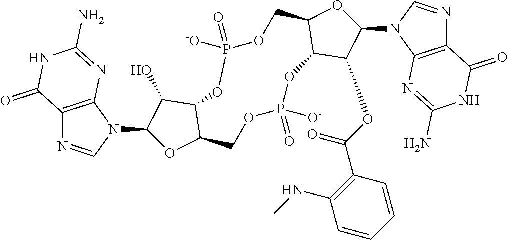 |
| T1-50 | 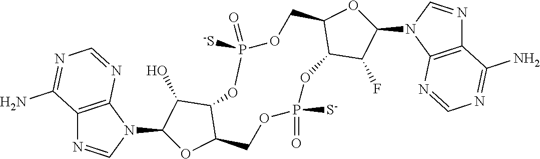 |
| T1-51 | 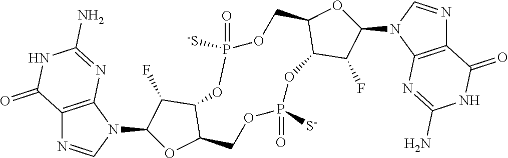 |
| T1-52 | 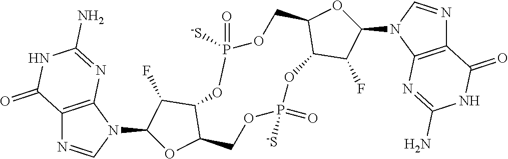 |
| T1-53 | 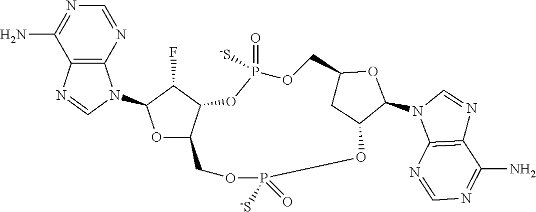 |
| T1-54 | 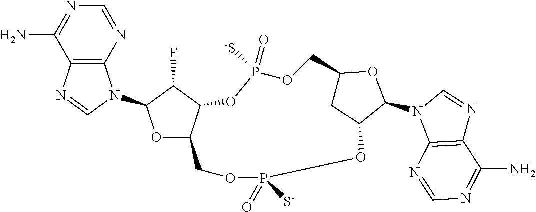 |
| T1-55 | 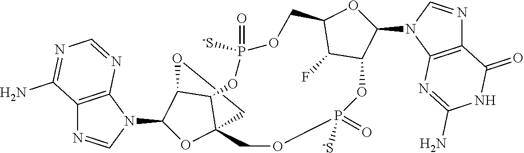 |
| T1-56 | 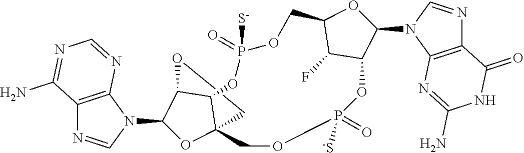 |
| T1-57 | 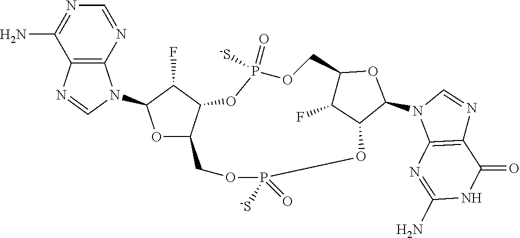 |
| T1-58 | 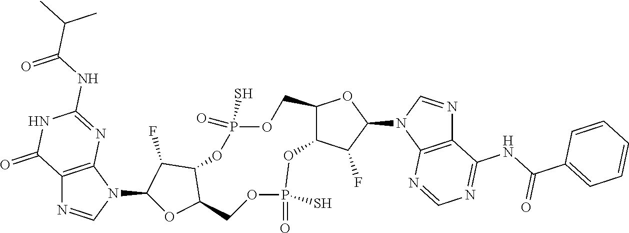 [SMILES: CC(C)C(=O)Nc1nc2c(ncn2[C@@H]2O[C@@H]3CO[P@@](=O)(S)O[C@H]4[C@@H](F)[C@H](n5cnc6c(NC(=O)c7ccccc7)ncnc65)O[C@@H]4CO[P@@](=O)(S)O[C@H]3[C@H]2F)c(=O)[nH]1] |
| T1-59 | 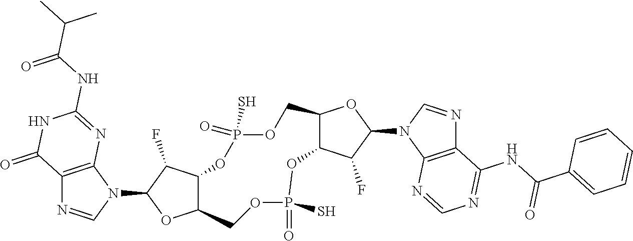 [SMILES: CC(C)C(=O)Nc1nc2c(ncn2[C@@H]2O[C@@H]3CO[P@](=O)(S)O[C@H]4[C@@H](F)[C@H](n5cnc6c(NC(=O)c7ccccc7)ncnc65)O[C@@H]4CO[P@@](=O)(S)O[C@H]3[C@H]2F)c(=O)[nH]1] |
| T1-60 | 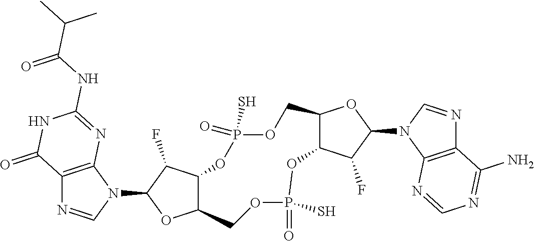 [SMILES: CC(C)C(=O)Nc1nc2c(ncn2[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)c(=O)[nH]1] |
| T1-61 | 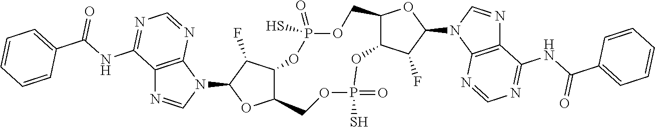 [SMILES: O=C(Nc1ncnc2c1ncn2[C@@H]1O[C@@H]2CO[P@@](=O)(S)O[C@H]3[C@@H](F)[C@H](n4cnc5c(NC(=O)c6ccccc6)ncnc54)O[C@@H]3CO[P@@](=O)(S)O[C@H]2[C@H]1F)c1ccccc1] |

### Embodiment 53

A Drug moiety (D) is a compound of Table 2:

TABLE 2

| Compound | |
|---|---|
| No. | Structure |
| T2-1 | 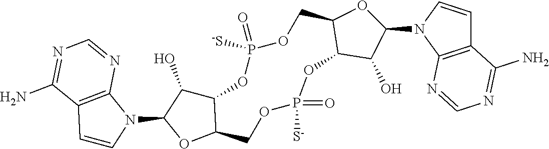 |
| T2-2 | 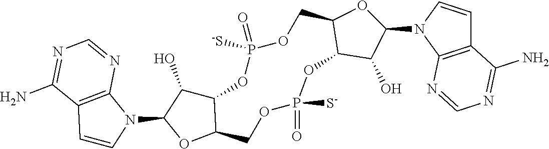 |
| T2-3 | 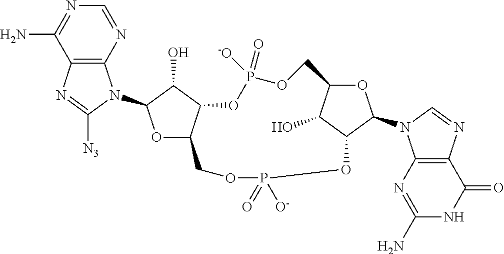 |
| T2-4 | 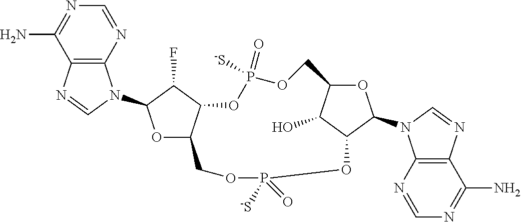 |
| T2-5 | 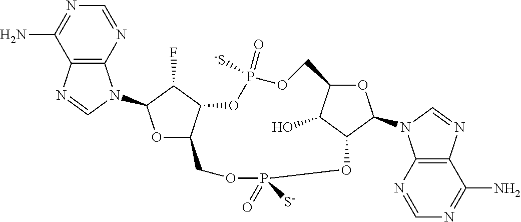 |
| T2-6 | 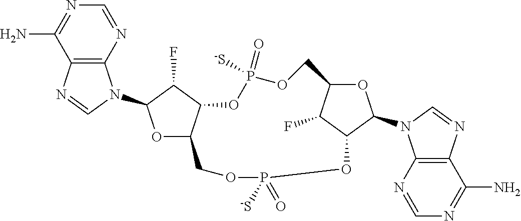 |
| T2-7 | 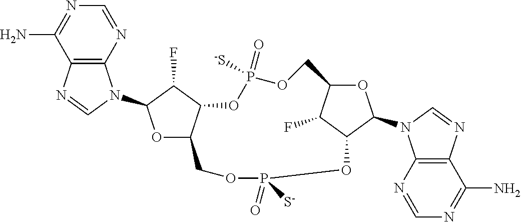 |
| T2-8 | 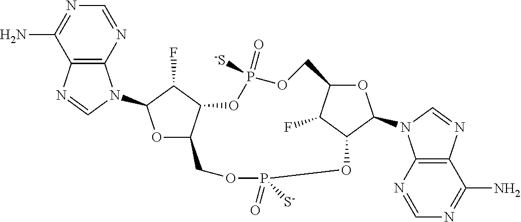 |
| T2-9 | 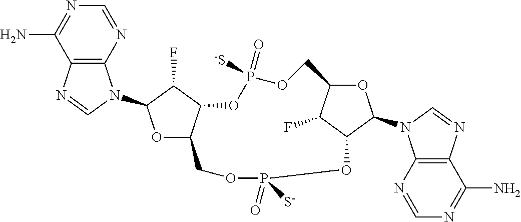 |
| T2-10 | 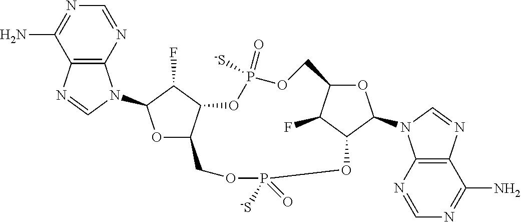 |
| T2-11 | 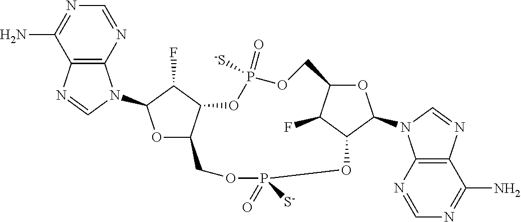 |
| T2-12 | 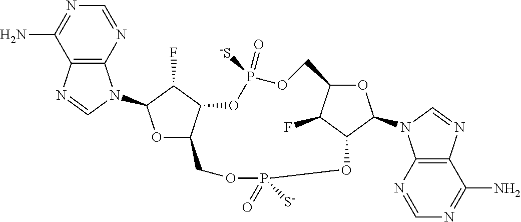 |
| T2-13 | 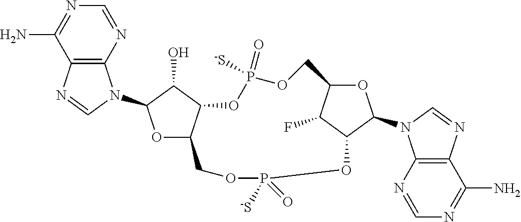 |
| T2-14 | 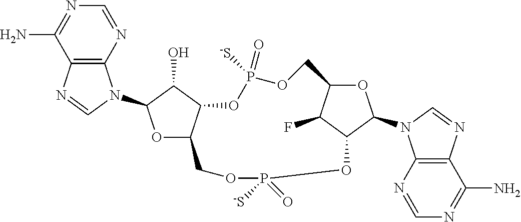 |
| T2-15 | 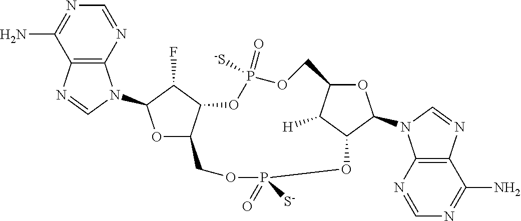 |
| T2-16 | 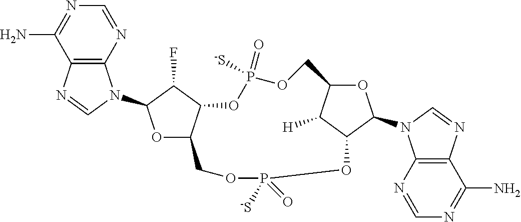 |
| T2-17 | 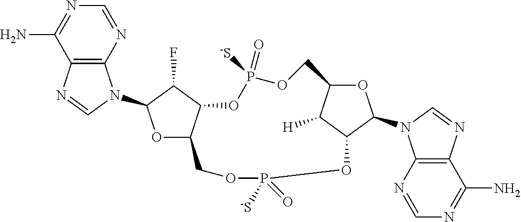 |
| T2-18 | 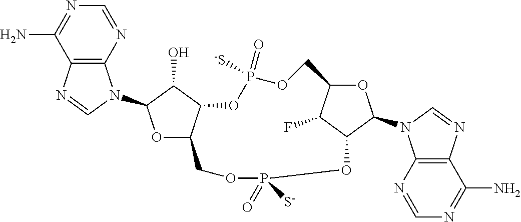 |
| T2-19 | 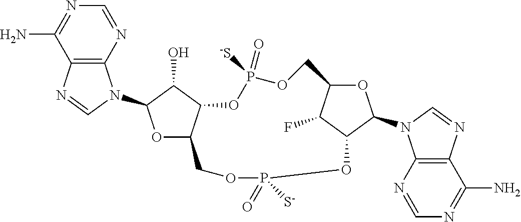 |
| T2-20 | 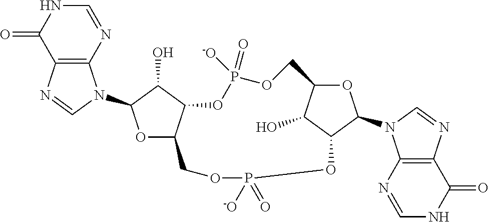 |
| T2-21 | 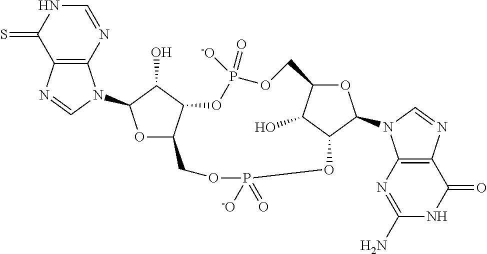 |
| T2-22 | 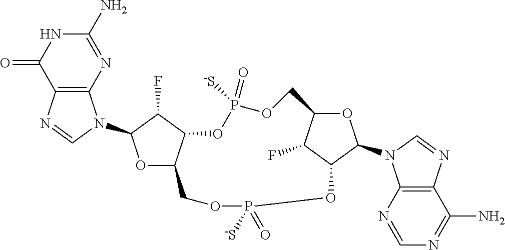 |
| T2-23 | 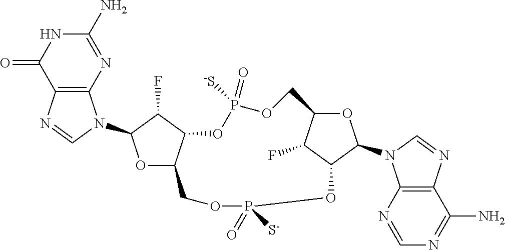 |
| T2-24 | 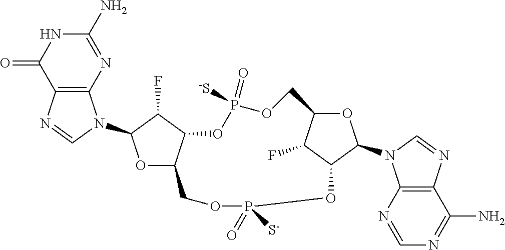 |
| T2-25 | 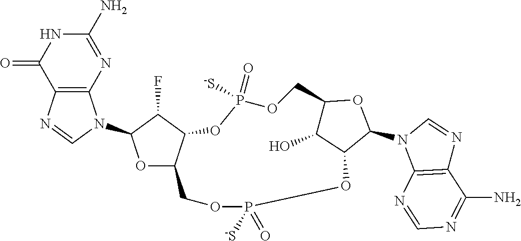 |
| T2-26 | 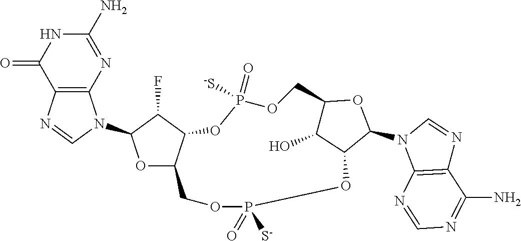 |
| T2-27 | 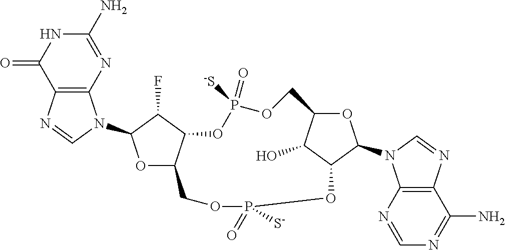 |
| T2-28 | 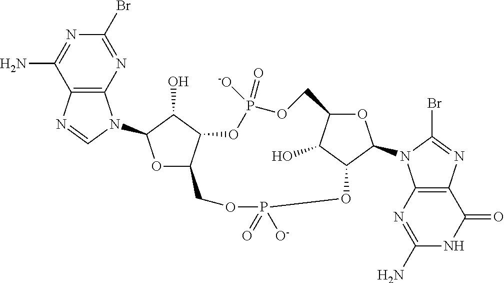 |
| T2-29 | 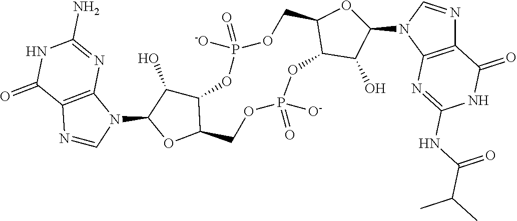 |
| T2-30 | 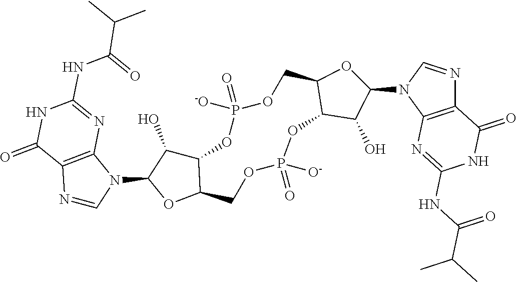 |
| T2-31 | 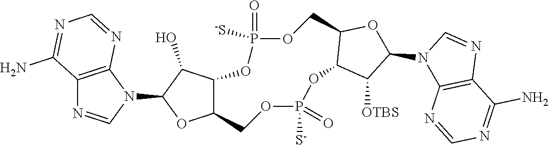 |
| T2-32 | 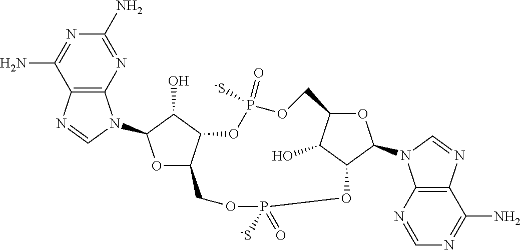 |
| T2-33 | 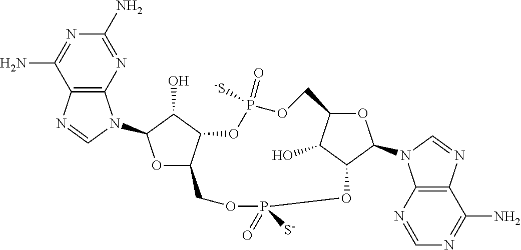 |
| T2-34 | 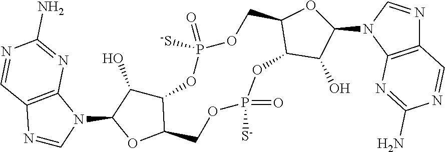 |
| T2-35 | 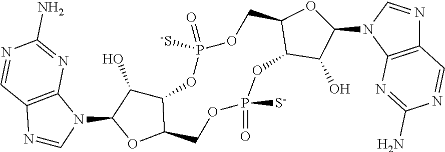 |
| T2-36 | 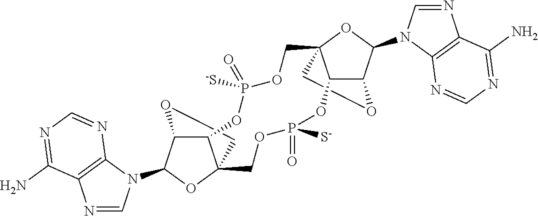 |
| T2-37 | 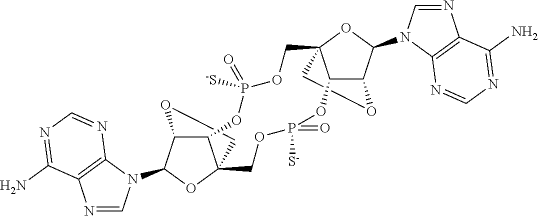 |
| T2-38 | 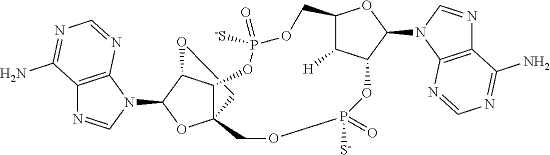 |
| T2-39 | 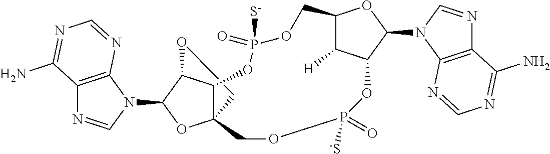 |
| T2-40 | 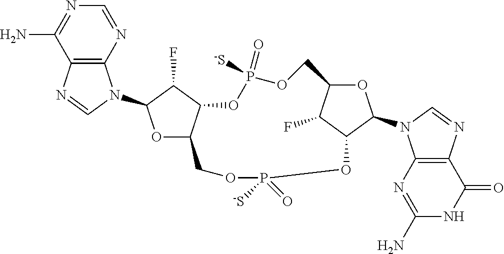 |
| T2-41 | 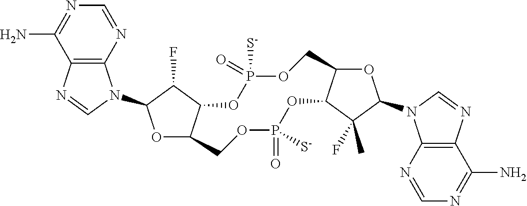 [SMILES: C[C@@]1(F)[C@@H]2O[P@](=O)([S-])OC[C@H]3O[C@@H](n4cnc5c(N)ncnc54)[C@H](F)[C@@H]3O[P@](=O)([S-])OC[C@H]2O[C@H]1n1cnc2c(N)ncnc21] |
| T2-42 | 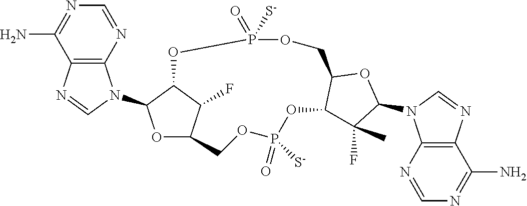 [SMILES: C[C@@]1(F)[C@@H]2O[P@](=O)([S-])OC[C@H]3O[C@@H](n4cnc5c(N)ncnc54)[C@H](O[P@](=O)([S-])OC[C@H]2O[C@H]1n1cnc2c(N)ncnc21)[C@@H]3F] |
| T2-43 | 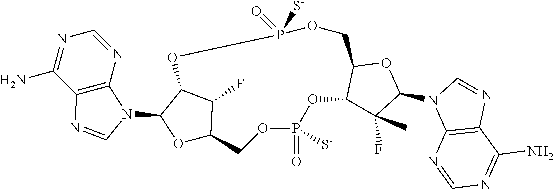 [SMILES: C[C@@]1(F)[C@@H]2O[P@](=O)([S-])OC[C@H]3O[C@@H](n4cnc5c(N)ncnc54)[C@H](O[P@@](=O)([S-])OC[C@H]2O[C@H]1n1cnc2c(N)ncnc21)[C@@H]3F] |
| T2-44 | 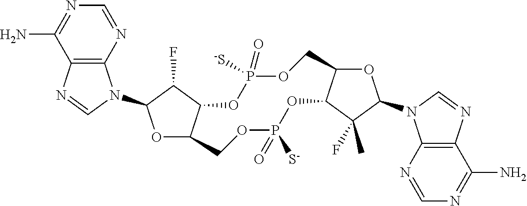 |
| T2-45 | 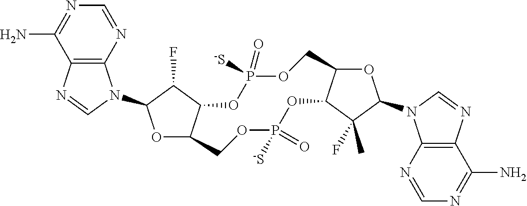 |
| T2-46 | 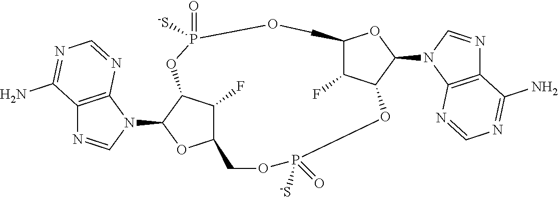 |
| T2-47 | 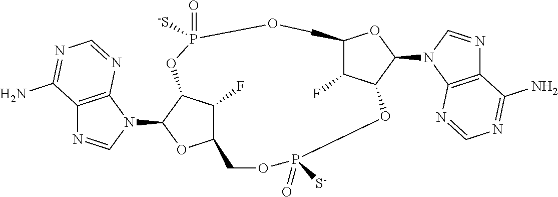 |
| T2-48 T1-57 | 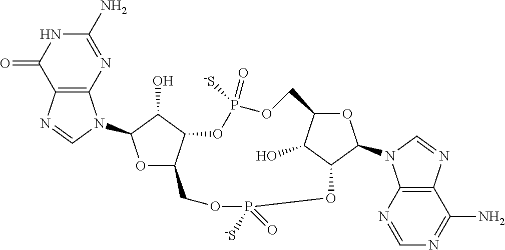 |
| T2-49 T1-58 | 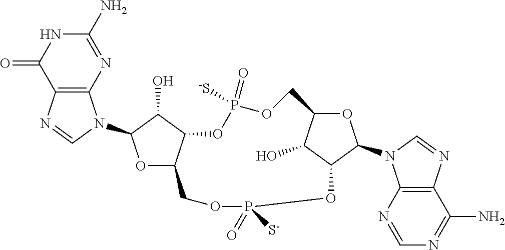 |
| T2-50 T1-59 | 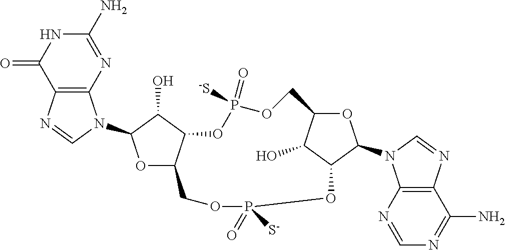 |
| T2-51 | 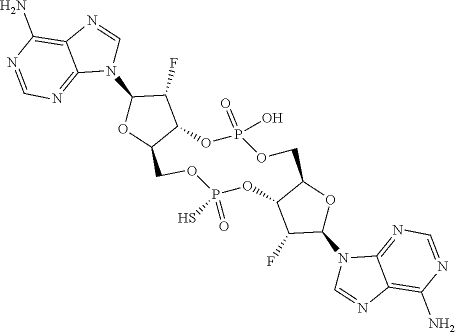 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F] |

### Embodiment 54

A Drug moiety (D) is a compound of Table 3:

TABLE 3

| Compound | |
|---|---|
| No. | Structure |
| T3-1 | 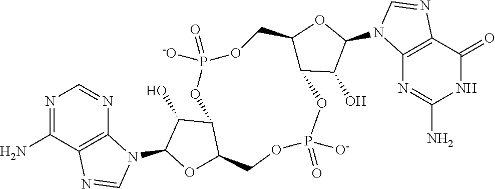 |
| T3-2 | 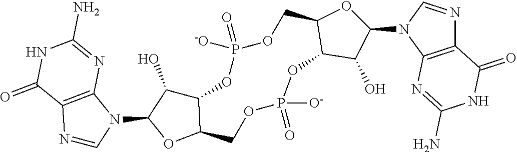 |
| T3-3 | 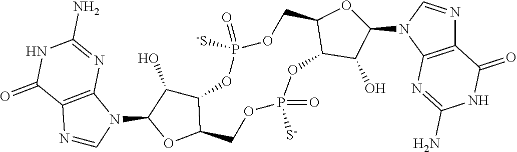 |
| T3-4 | 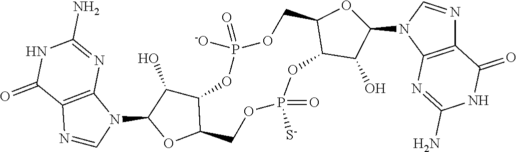 |
| T3-5 | 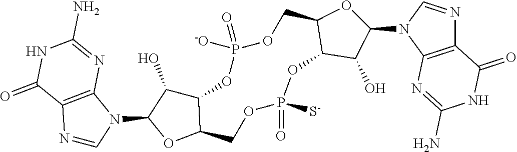 |
| T3-6 | 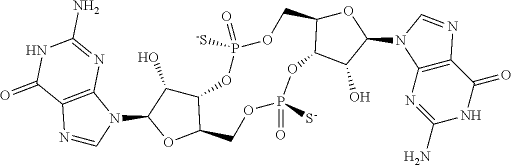 |
| T3-7 | 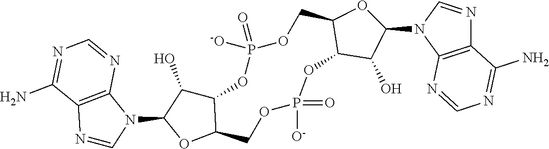 |
| T3-8 | 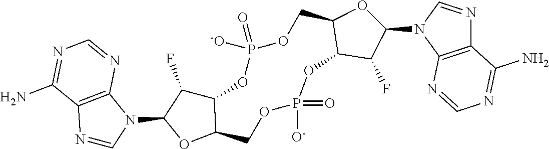 |
| T3-9 | 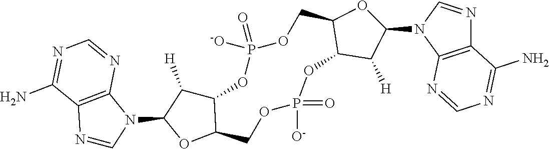 |
| T3-10 | 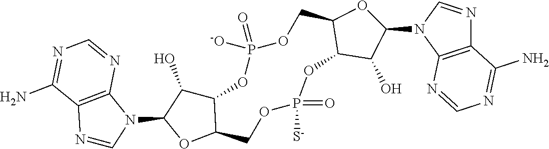 |
| T3-11 | 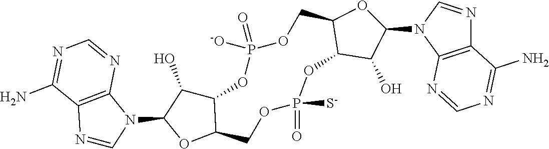 |
| T3-12 | 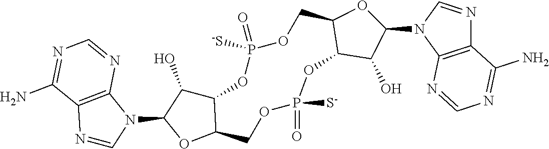 |
| T3-13 | 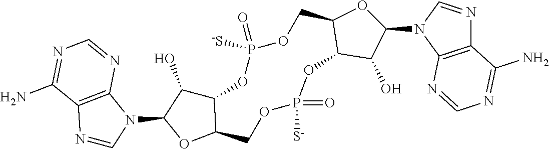 |
| T3-14 | 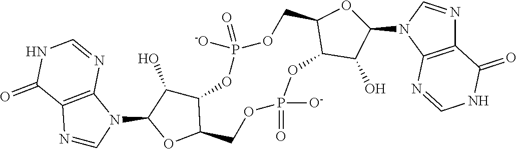 |
| T3-15 | 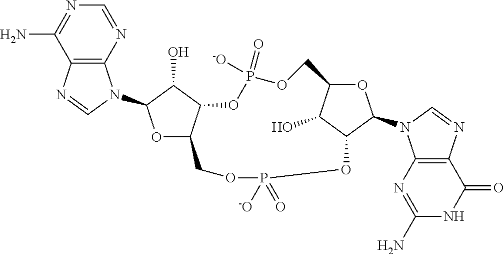 |
| T3-16 | 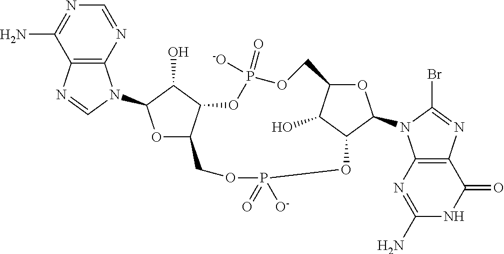 |
| T3-17 | 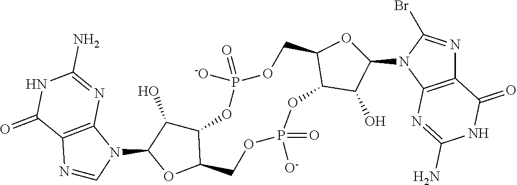 |
| T3-18 | 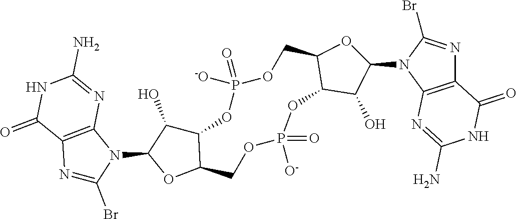 |
| T3-19 | 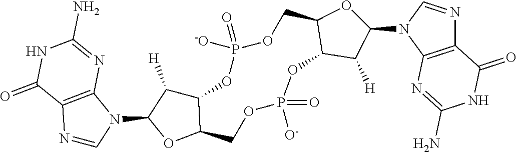 |
| T3-20 | 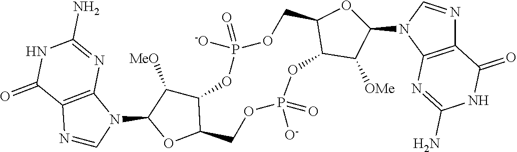 |
| T3-21 | 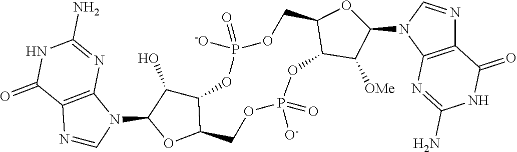 |

### Embodiment 55

The Drug moiety (D) is

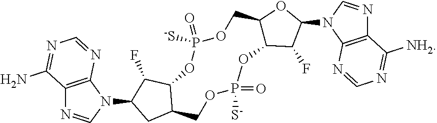

### Embodiment 56

The Drug moiety (D) is

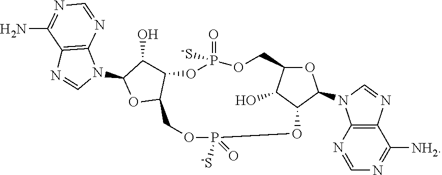

### Embodiment 57

The Drug moiety (D) is

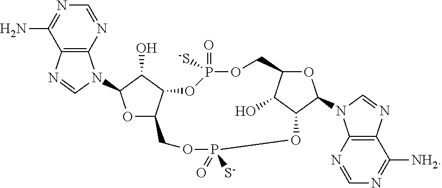

### Embodiment 58

The Drug moiety (D) is

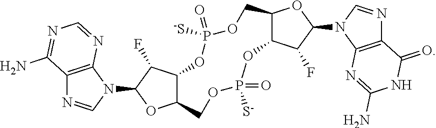

### Embodiment 59

The Drug moiety (D) is

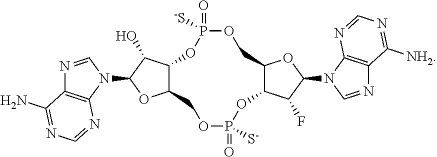

### Embodiment 60

The Drug moiety (D) is

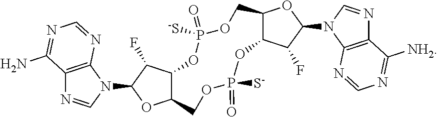

### Embodiment 61

The Drug moiety (D) is

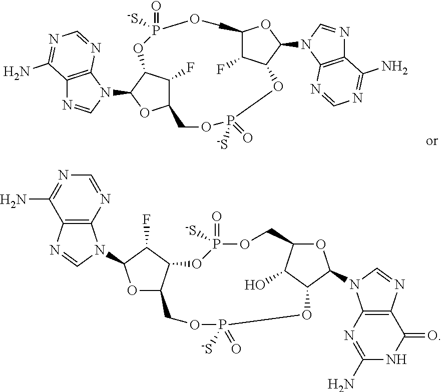

### Embodiment 62

The Drug moiety (D) is

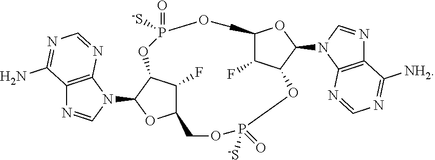

### Embodiment 63

The Drug moiety (D) is

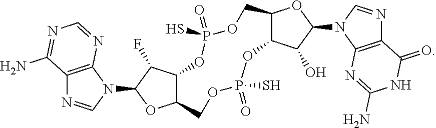 [SMILES: Nc1nc2c(ncn2[C@@H]2O[C@@H]3CO[P@](=O)(S)O[C@H]4[C@@H](F)[C@H](n5cnc6c(N)ncnc65)O[C@@H]4CO[P@@](=O)(S)O[C@H]3[C@H]2O)c(=O)[nH]1]

### Embodiment 64

The Drug moiety (D) is

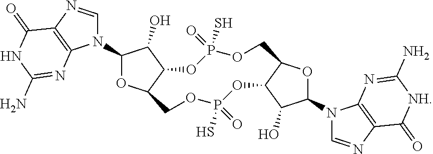 [SMILES: Nc1nc2c(ncn2[C@@H]2O[C@@H]3CO[P@@](=O)(S)O[C@H]4[C@@H](O)[C@H](n5cnc6c(=O)[nH]c(N)nc65)O[C@@H]4CO[P@@](=O)(S)O[C@H]3[C@H]2O)c(=O)[nH]1]

### Embodiment 65

The Drug moiety (D) is

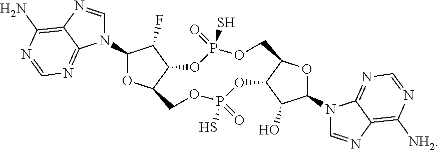 [SMILES: Nc1ncnc2c1ncn2[C@@H]1O[C@@H]2CO[P@](=O)(S)O[C@H]3[C@@H](F)[C@H](n4cnc5c(N)ncnc54)O[C@@H]3CO[P@@](=O)(S)O[C@H]2[C@H]1O]

### Embodiment 66

The Drug moiety (D) is

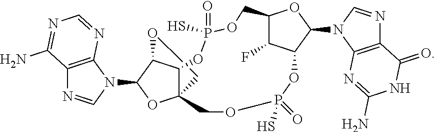 [SMILES: Nc1nc2c(ncn2[C@@H]2O[C@@H]3CO[P@@](=O)(S)O[C@H]4[C@H]5OC[C@]4(CO[P@@](=O)(S)O[C@@H]2[C@@H]3F)O[C@H]5n2cnc3c(N)ncnc32)c(=O)[nH]1]

### Embodiment 67

The Drug moiety (D) is

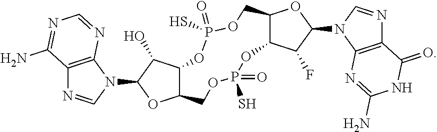 [SMILES: Nc1nc2c(ncn2[C@@H]2O[C@@H]3CO[P@@](=O)(S)O[C@H]4[C@@H](O)[C@H](n5cnc6c(N)ncnc65)O[C@@H]4CO[P@](=O)(S)O[C@H]3[C@H]2F)c(=O)[nH]1]

### Embodiment 68

The Drug moiety (D) is

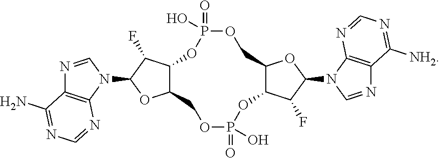 [SMILES: Nc1ncnc2c1ncn2[C@@H]1O[C@@H]2COP(=O)(O)O[C@H]3[C@@H](F)[C@H](n4cnc5c(N)ncnc54)O[C@@H]3COP(=O)(O)O[C@H]2[C@H]1F]

### Embodiment 69

The Drug moiety (D) is

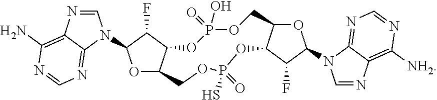 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F]

In another aspect the Drug moiety (D) of the immunoconjugates of the invention are the compounds disclosed in Aduro (WO2016/145102).

In another aspect the Drug moiety (D) of the immunoconjugates of the invention are the compounds disclosed in Aduro Biotech (WO2014/093936).

In another aspect the Drug moiety (D) of the immunoconjugates of the invention are the compounds disclosed in Aduro and Novartis unpublished US Provisional application U.S. Ser. No. 62/362,907 filed Jul. 15, 2016.

In another aspect the Drug moiety (D) of the immunoconjugates of the invention are the compounds disclosed in Aduro and Novartis unpublished PCT application PCT/US2016/059506 filed 28 Oct. 2016.

In another aspect the Drug moiety (D) of the immunoconjugates of the invention are the compounds disclosed in Memorial Sloan Kettering et al (WO2014/179335). Such compounds are listed in Table 4.

In another aspect the Drug moiety (D) of the immunoconjugates of the invention are the compounds disclosed in Merck & Co (WO2017/027646). Such compounds are listed in Table 4.

In another aspect the Drug moiety (D) of the immunoconjugates of the invention are the compounds disclosed in Merck & Co (WO2017/027645). Such compounds are listed in Table 4.

In another aspect the Drug moiety (D) of the immunoconjugates of the invention are the compounds disclosed in GlaxoSmithKline (WO2015/185565). Such compounds are listed in Table 4.

In another aspect the Drug moiety (D) of the immunoconjugates of the invention are the compounds disclosed in Brock University (WO2015/074145). Such compounds are listed in Table 4.

In another aspect the Drug moiety (D) of the immunoconjugates of the invention are the compounds disclosed in Rutgers (U.S. Pat. No. 9,315,523). Such compounds are listed in Table 4.

In another aspect the Drug moiety (D) of the immunoconjugates of the invention are the compounds disclosed in Spring Bank (WO2007070598, WO2017004499 and WO2017011622).

Such compounds are listed in Table 4.

In another aspect the Drug moiety (D) of the immunoconjugates of the invention are the compounds disclosed in Invivogen (WO2016/096174. Such compounds are listed in Table 4.

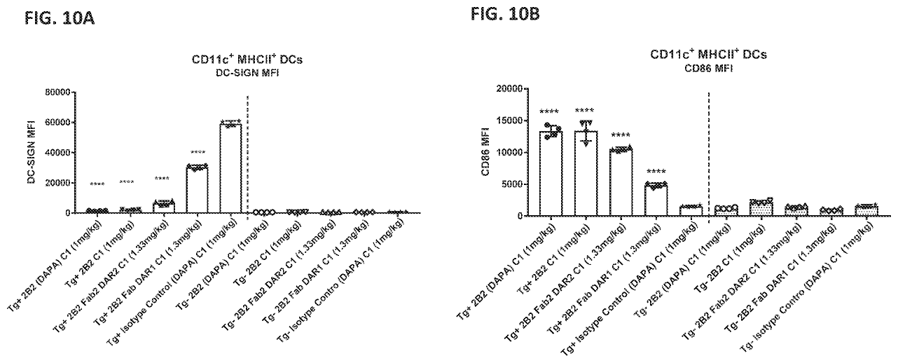

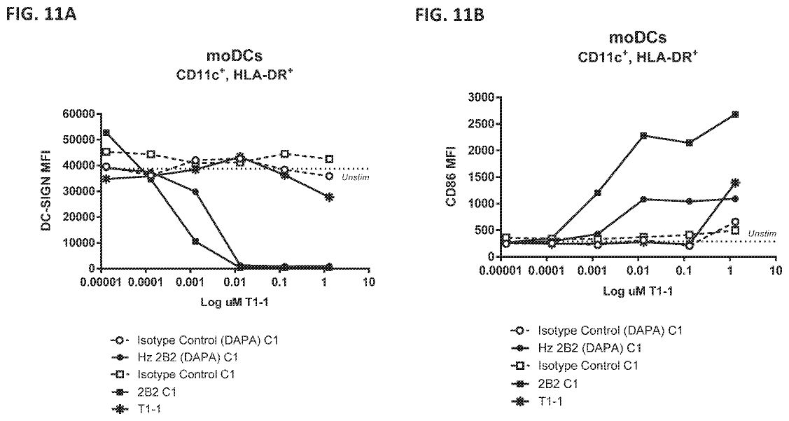

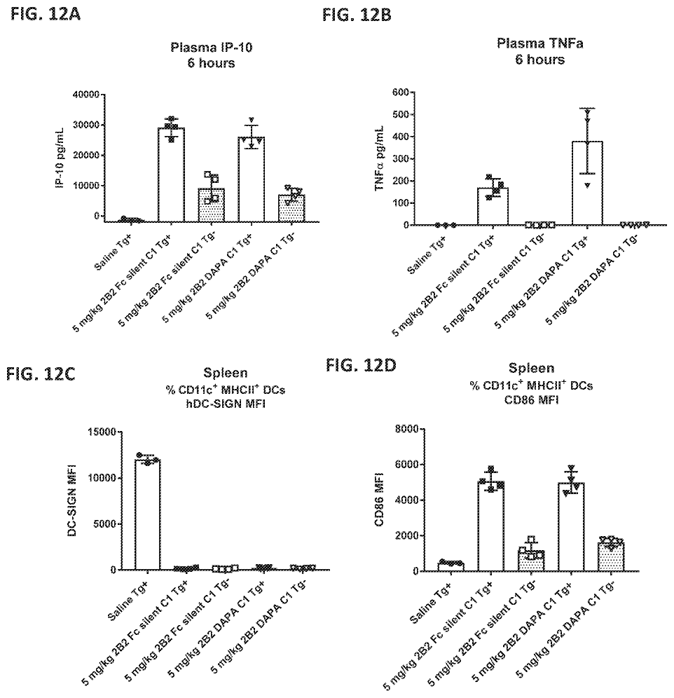

In another aspect the Drug moiety (D) of the immunoconjugates of the invention are the compounds disclosed in Regents of Univ. California and Aduro Biotech (WO2014/189805). Such compounds are disclosed herein in FIG. 10, FIG. 11, and FIG. 12.

In another aspect the Drug moiety (D) of the immunoconjugates of the invention are the compounds disclosed in Sperovie (WO2018009648).

In another aspect the Drug moiety (D) of the immunoconjugates of the invention are the compounds disclosed in Sperovie (WO2018009652).

In another aspect the Drug moiety (D) of the immunoconjugates of the invention are the compounds disclosed in Sperovie (WO2018013887).

In another aspect the Drug moiety (D) of the immunoconjugates of the invention are the compounds disclosed in Sperovie (WO2018013908).Each of the preceding applications are incorporated by reference in their entirety.

TABLE 4

| Ex. | |
|---|---|
| No. | Structure |
| T4-1 | 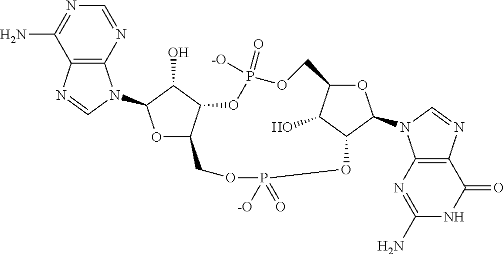 |
| T4-2 | 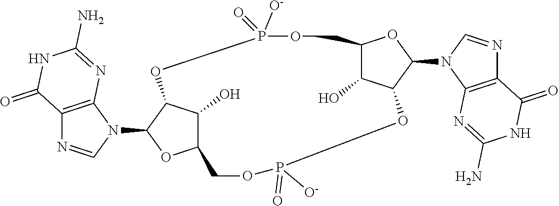 [SMILES: Nc1nc2c(ncn2[C@@H]2O[C@@H]3COP(=O)([O-])O[C@@H]4[C@H](O)[C@@H](COP(=O)([O-])O[C@@H]2[C@@H]3O)O[C@H]4n2cnc3c(=O)[nH]c(N)nc32)c(=O)[nH]1] |
| T4-3 | 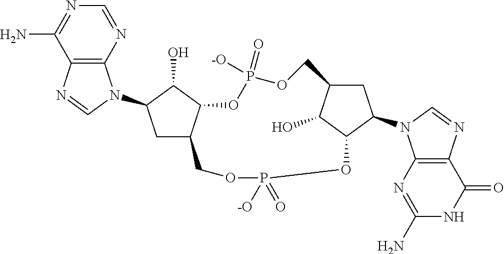 |
| T4-4 | 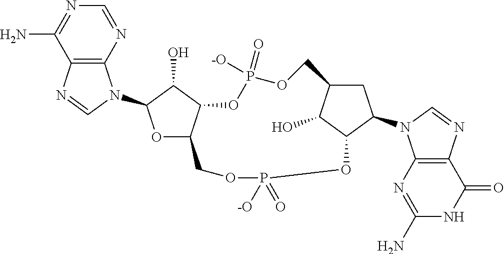 |
| T4-5 | 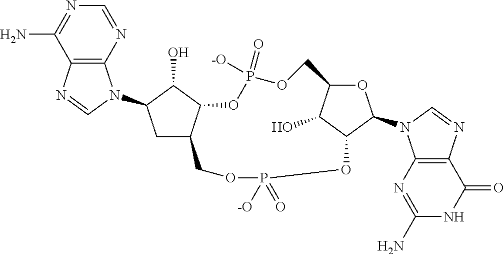 |
| T4-6 | 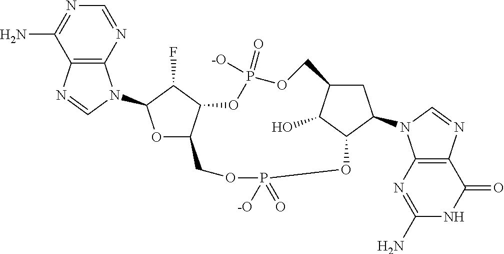 |
| T4-7 | 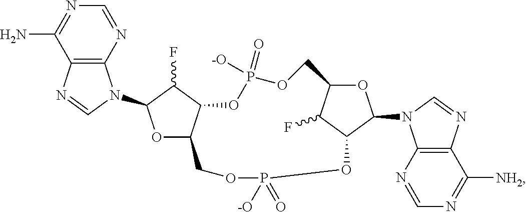 |
| | as RR, RS, SR and SS diastereomers |
| T4-8 | 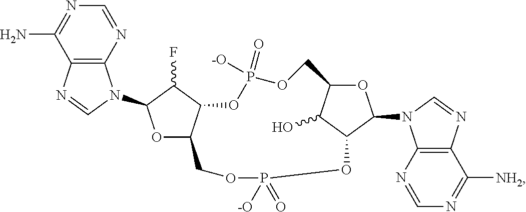 |
| | as RR, RS, SR and SS diastereomers |
| T4-9 | 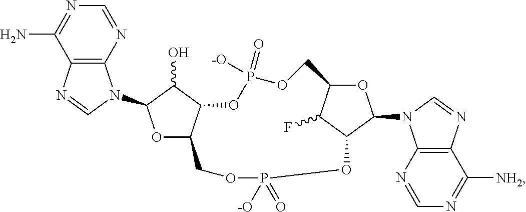 |
| | as RR, RS, SR and SS diastereomers |
| T4-10 | 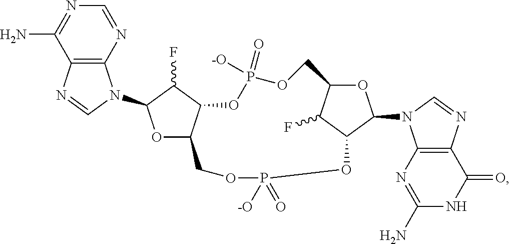 |
| | as RR, RS, SR and SS diastereomers |
| T4-11 | 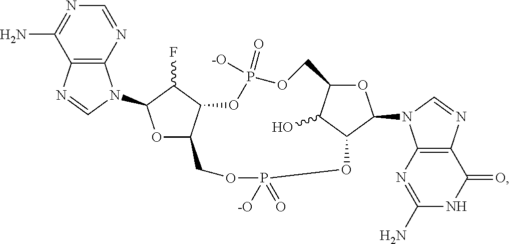 |
| | as RR, RS, SR and SS diastereomers |
| T4-12 | 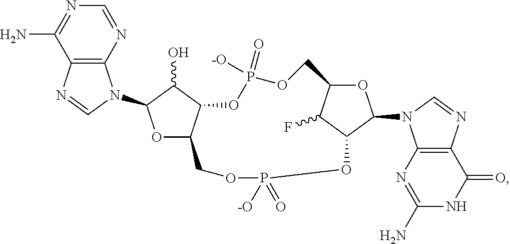 |
| | as RR, RS, SR and SS diastereomers |
| T4-13 | 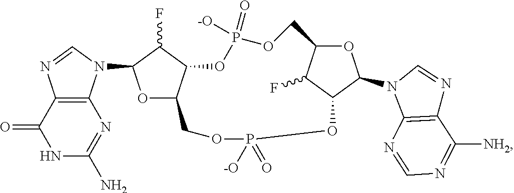 |
| | as RR, RS, SR and SS diastereomers |
| T4-14 | 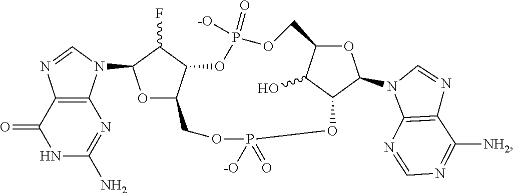 |
| | as RR, RS, SR and SS diastereomers |
| T4-15 | 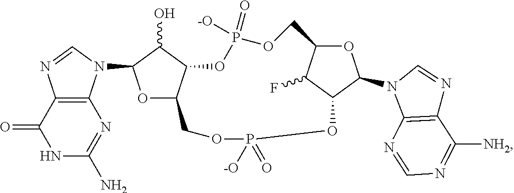 |
| | as RR, RS, SR and SS diastereomers |
| T4-16 | 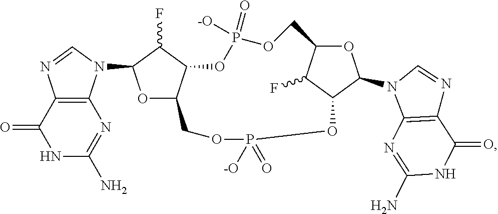 |
| | as RR, RS, SR and SS diastereomers |
| T4-17 | 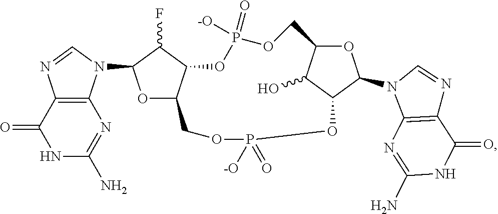 |
| | as RR, RS, SR and SS diastereomers |
| T4-18 | 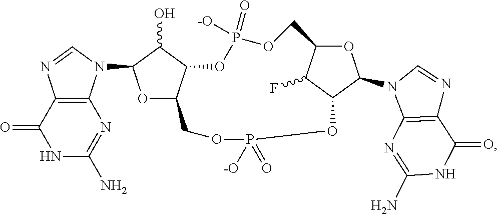 |
| | as RR, RS, SR and SS diastereomers |
| T4-19 | 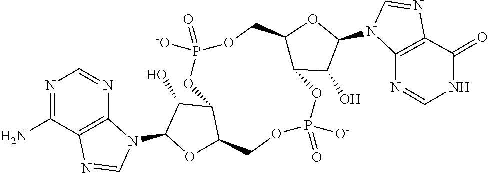 |
| T4-20 | 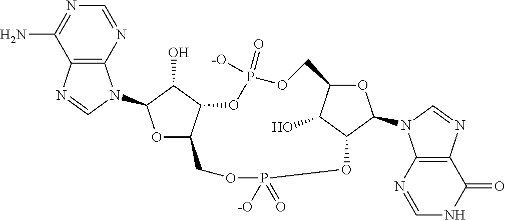 |
| T4-21 | 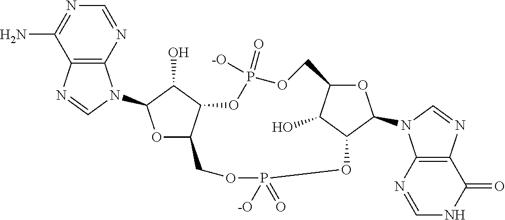 |
| T4-22 | 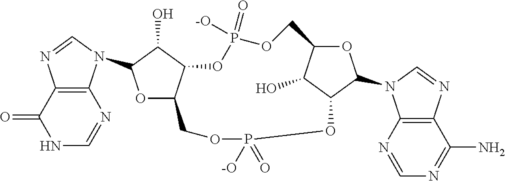 |
| T4-23 | 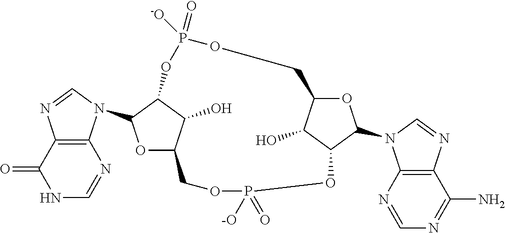 |
| T4-24 | 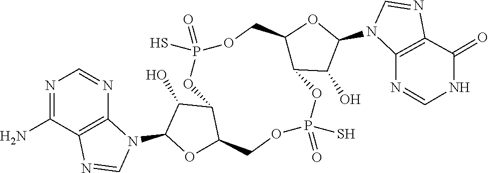 [SMILES: Nc1ncnc2c1ncn2[C@@H]1O[C@@H]2COP(=O)(S)O[C@H]3[C@@H](O)[C@H](n4cnc5c(=O)[nH]cnc54)O[C@@H]3COP(=O)(S)O[C@H]2[C@H]1O] |
| T4-25 | 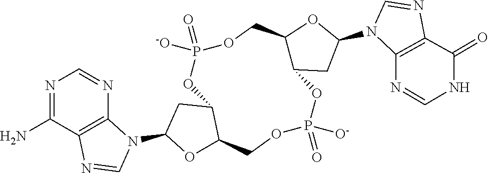 |
| T4-26 | 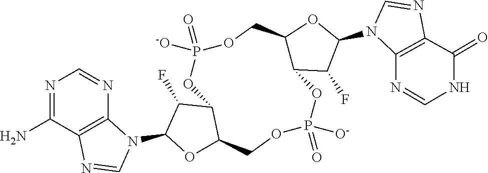 |
| T4-27 | 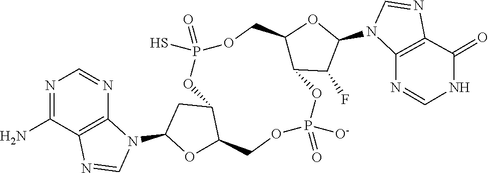 [SMILES: Nc1ncnc2c1ncn2[C@H]1C[C@@H]2OP(=O)(S)OC[C@H]3O[C@@H](n4cnc5c(=O)[nH]cnc54)[C@H](F)[C@@H]3OP(=O)([O-])OC[C@H]2O1] |
| T4-28 | 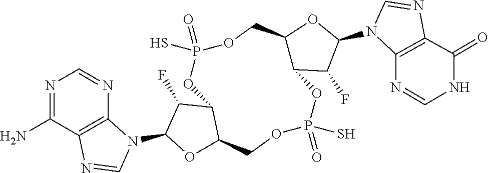 [SMILES: Nc1ncnc2c1ncn2[C@@H]1O[C@@H]2COP(=O)(S)O[C@H]3[C@@H](F)[C@H](n4cnc5c(=O)[nH]cnc54)O[C@@H]3COP(=O)(S)O[C@H]2[C@H]1F] |
| T4-29 | 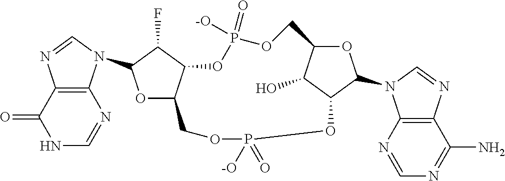 |
| T4-30 | 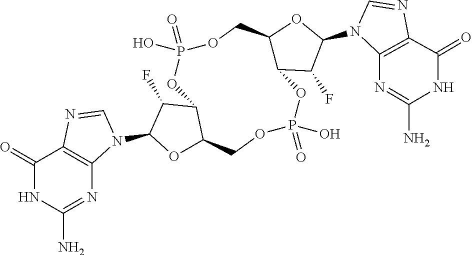 [SMILES: Nc1nc2c(ncn2[C@@H]2O[C@@H]3COP(=O)(O)O[C@H]4[C@@H](F)[C@H](n5cnc6c(=O)[nH]c(N)nc65)O[C@@H]4COP(=O)(O)O[C@H]3[C@H]2F)c(=O)[nH]1] |
| T4-31 | 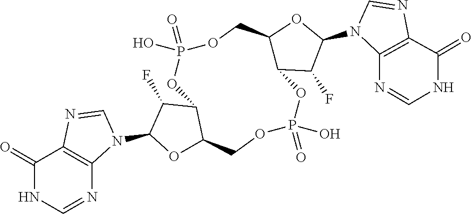 [SMILES: O=c1[nH]cnc2c1ncn2[C@@H]1O[C@@H]2COP(=O)(O)O[C@H]3[C@@H](F)[C@H](n4cnc5c(=O)[nH]cnc54)O[C@@H]3COP(=O)(O)O[C@H]2[C@H]1F] |
| T4-32 | 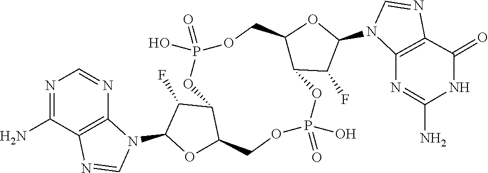 [SMILES: Nc1nc2c(ncn2[C@@H]2O[C@@H]3COP(=O)(O)O[C@H]4[C@@H](F)[C@H](n5cnc6c(N)ncnc65)O[C@@H]4COP(=O)(O)O[C@H]3[C@H]2F)c(=O)[nH]1] |
| T4-33 | 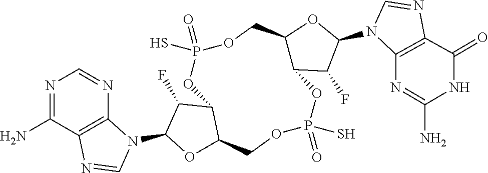 [SMILES: Nc1nc2c(ncn2[C@@H]2O[C@@H]3COP(=O)(S)O[C@H]4[C@@H](F)[C@H](n5cnc6c(N)ncnc65)O[C@@H]4COP(=O)(S)O[C@H]3[C@H]2F)c(=O)[nH]1] |
| T4-34 | 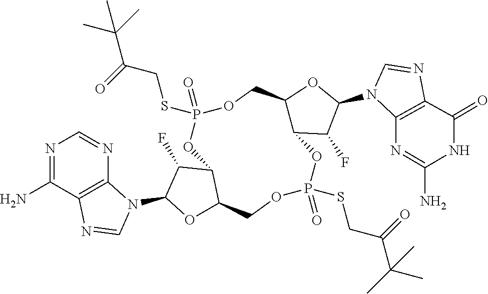 [SMILES: CC(C)(C)C(=O)CSP1(=O)OC[C@H]2O[C@@H](n3cnc4c(=O)[nH]c(N)nc43)[C@H](F)[C@@H]2OP(=O)(SCC(=O)C(C)(C)C)OC[C@H]2O[C@@H](n3cnc4c(N)ncnc43)[C@H](F)[C@@H]2O1] |
| T4-35 | 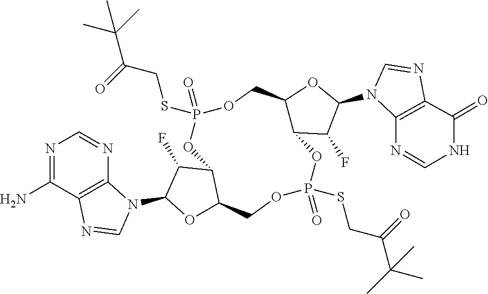 [SMILES: CC(C)(C)C(=O)CSP1(=O)OC[C@H]2O[C@@H](n3cnc4c(=O)[nH]cnc43)[C@H](F)[C@@H]2OP(=O)(SCC(=O)C(C)(C)C)OC[C@H]2O[C@@H](n3cnc4c(N)ncnc43)[C@H](F)[C@@H]2O1] |
| T4-36 | 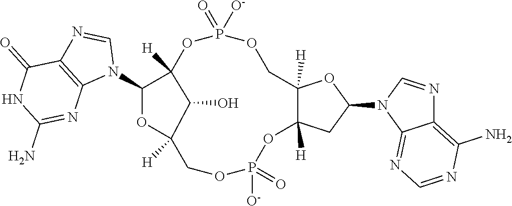 [SMILES: Nc1nc2c(ncn2[C@@H]2O[C@@H]3COP(=O)([O-])O[C@H]4C[C@H](n5cnc6c(N)ncnc65)O[C@@H]4COP(=O)([O-])O[C@@H]2[C@@H]3O)c(=O)[nH]1] |
| T4-37 | 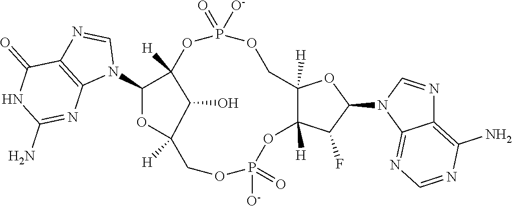 [SMILES: Nc1nc2c(ncn2[C@@H]2O[C@@H]3COP(=O)([O-])O[C@H]4[C@@H](F)[C@H](n5cnc6c(N)ncnc65)O[C@@H]4COP(=O)([O-])O[C@@H]2[C@@H]3O)c(=O)[nH]1] |
| T4-38 | 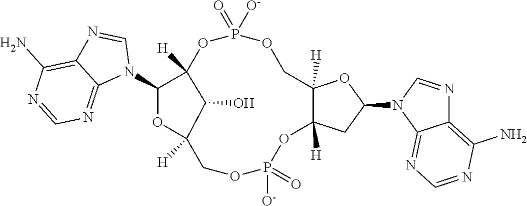 [SMILES: Nc1ncnc2c1ncn2[C@@H]1O[C@@H]2COP(=O)([O-])O[C@H]3C[C@H](n4cnc5c(N)ncnc54)O[C@@H]3COP(=O)([O-])O[C@@H]1[C@@H]2O] |
| T4-39 | 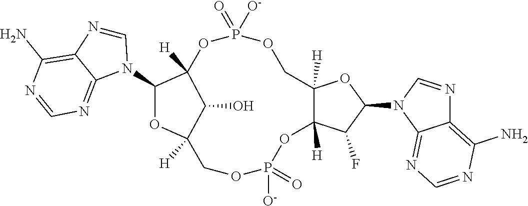 [SMILES: Nc1ncnc2c1ncn2[C@@H]1O[C@@H]2COP(=O)([O-])O[C@@H]3[C@H](O)[C@@H](COP(=O)([O-])O[C@H]2[C@H]1F)O[C@H]3n1cnc2c(N)ncnc21] |
| T4-40 | 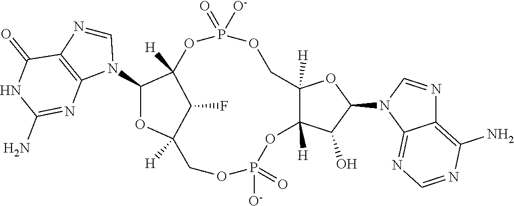 [SMILES: Nc1nc2c(ncn2[C@@H]2O[C@@H]3COP(=O)([O-])O[C@H]4[C@@H](O)[C@H](n5cnc6c(N)ncnc65)O[C@@H]4COP(=O)([O-])O[C@@H]2[C@@H]3F)c(=O)[nH]1] |
| T4-41 | 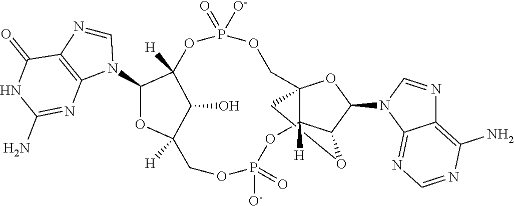 [SMILES: Nc1nc2c(ncn2[C@@H]2O[C@@H]3COP(=O)([O-])O[C@H]4[C@H]5OC[C@]4(COP(=O)([O-])O[C@@H]2[C@@H]3O)O[C@H]5n2cnc3c(N)ncnc32)c(=O)[nH]1] |
| T4-42 | 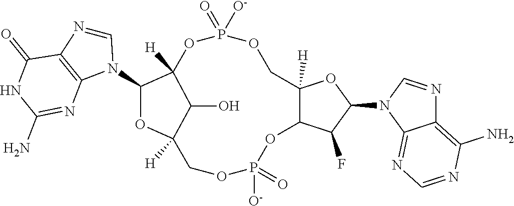 [SMILES: Nc1nc2c(ncn2[C@@H]2O[C@@H]3COP(=O)([O-])OC4[C@@H](COP(=O)([O-])O[C@@H]2C3O)O[C@@H](n2cnc3c(N)ncnc32)[C@H]4F)c(=O)[nH]1] |
| T4-43 | 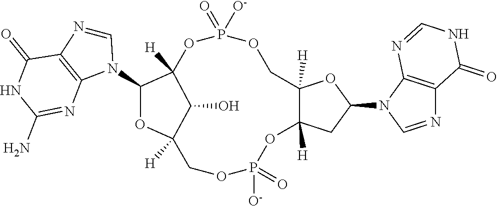 [SMILES: Nc1nc2c(ncn2[C@@H]2O[C@@H]3COP(=O)([O-])O[C@H]4C[C@H](n5cnc6c(=O)[nH]cnc65)O[C@@H]4COP(=O)([O-])O[C@@H]2[C@@H]3O)c(=O)[nH]1] |
| T4-44 | 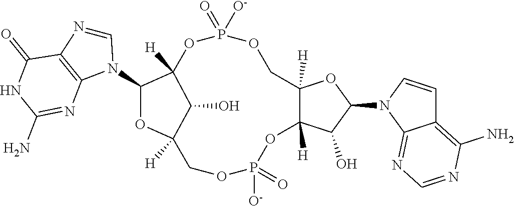 [SMILES: Nc1nc2c(ncn2[C@@H]2O[C@@H]3COP(=O)([O-])O[C@H]4[C@@H](O)[C@H](n5ccc6c(N)ncnc65)O[C@@H]4COP(=O)([O-])O[C@@H]2[C@@H]3O)c(=O)[nH]1] |
| T4-45 | 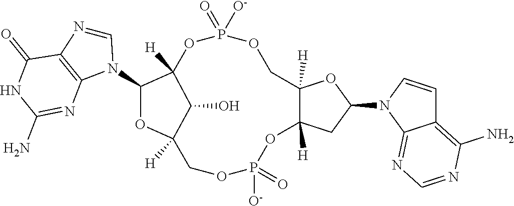 [SMILES: Nc1nc2c(ncn2[C@@H]2O[C@@H]3COP(=O)([O-])O[C@H]4C[C@H](n5ccc6c(N)ncnc65)O[C@@H]4COP(=O)([O-])O[C@@H]2[C@@H]3O)c(=O)[nH]1] |
| T4-46 | 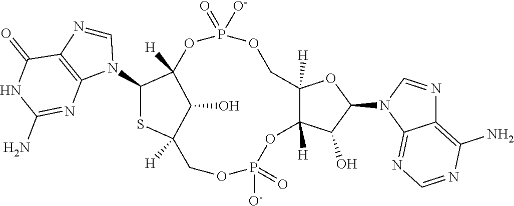 [SMILES: Nc1nc2c(ncn2[C@@H]2S[C@@H]3COP(=O)([O-])O[C@H]4[C@@H](O)[C@H](n5cnc6c(N)ncnc65)O[C@@H]4COP(=O)([O-])O[C@@H]2[C@@H]3O)c(=O)[nH]1] |
| T4-47 | 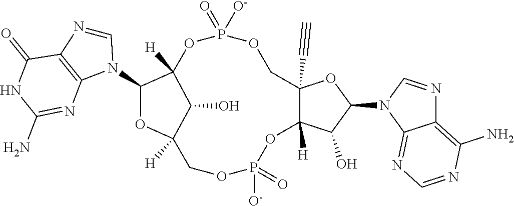 [SMILES: C#C[C@@]12COP(=O)([O-])O[C@@H]3[C@H](O)[C@@H](COP(=O)([O-])O[C@H]1[C@@H](O)[C@H](n1cnc4c(N)ncnc41)O2)O[C@H]3n1cnc2c(=O)[nH]c(N)nc21] |
| T4-48 | 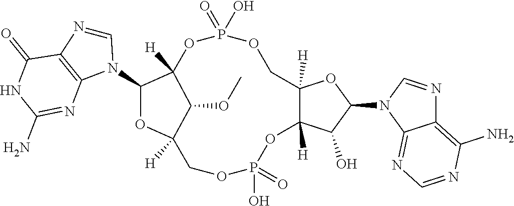 [SMILES: CO[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21] |
| T4-49 | 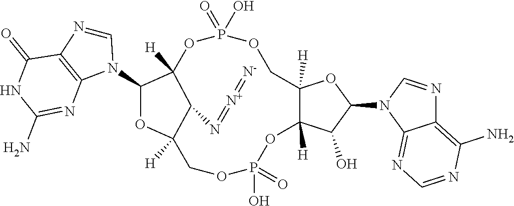 [SMILES: [N-]=[N+]=N[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21] |
| T4-50 | 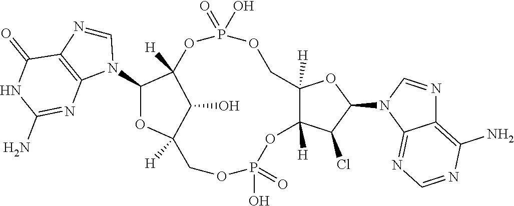 [SMILES: Nc1nc2c(ncn2[C@@H]2O[C@@H]3COP(=O)(O)O[C@H]4[C@H](Cl)[C@H](n5cnc6c(N)ncnc65)O[C@@H]4COP(=O)(O)O[C@@H]2[C@@H]3O)c(=O)[nH]1] |
| T4-51 | 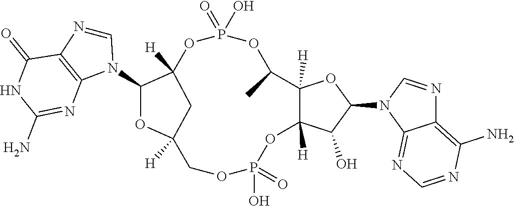 [SMILES: C[C@H]1OP(=O)(O)O[C@@H]2C[C@@H](COP(=O)(O)O[C@H]3[C@@H](O)[C@H](n4cnc5c(N)ncnc54)O[C@@H]31)O[C@H]2n1cnc2c(=O)[nH]c(N)nc21] |
| T4-52 | 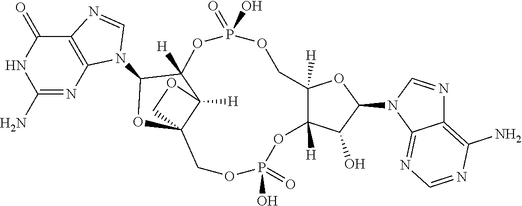 [SMILES: Nc1nc2c(ncn2[C@@H]2O[C@@]34CO[C@H]3[C@H]2O[P@@](=O)(O)OC[C@H]2O[C@@H](n3cnc5c(N)ncnc53)[C@H](O)[C@@H]2O[P@@](=O)(O)OC4)c(=O)[nH]1] |
| T4-53 | 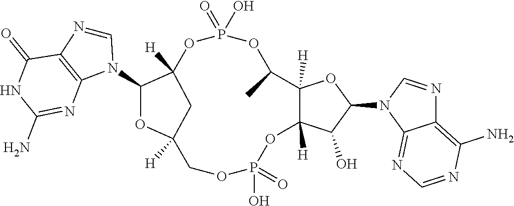 [SMILES: C[C@H]1OP(=O)(O)O[C@@H]2C[C@@H](COP(=O)(O)O[C@H]3[C@@H](O)[C@H](n4cnc5c(N)ncnc54)O[C@@H]31)O[C@H]2n1cnc2c(=O)[nH]c(N)nc21] |
| T4-54 | 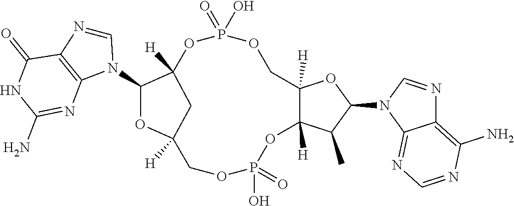 [SMILES: C[C@H]1[C@@H]2OP(=O)(O)OC[C@@H]3C[C@@H](OP(=O)(O)OC[C@H]2O[C@H]1n1cnc2c(N)ncnc21)[C@H](n1cnc2c(=O)[nH]c(N)nc21)O3] |
| T4-55 | 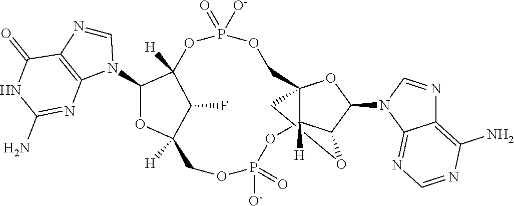 [SMILES: Nc1nc2c(ncn2[C@@H]2O[C@@H]3COP(=O)([O-])O[C@H]4[C@H]5OC[C@]4(COP(=O)([O-])O[C@@H]2[C@@H]3F)O[C@H]5n2cnc3c(N)ncnc32)c(=O)[nH]1] |
| T4-56 | 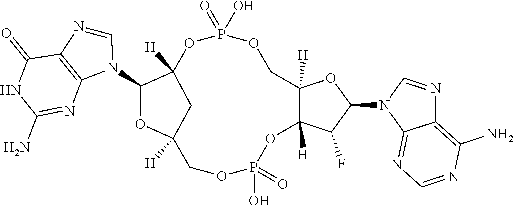 [SMILES: Nc1nc2c(ncn2[C@@H]2O[C@@H]3COP(=O)(O)O[C@H]4[C@@H](F)[C@H](n5cnc6c(N)ncnc65)O[C@@H]4COP(=O)(O)O[C@@H]2C3)c(=O)[nH]1] |
| T4-57 | 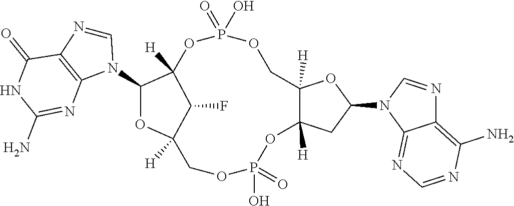 [SMILES: Nc1nc2c(ncn2[C@@H]2O[C@@H]3COP(=O)(O)O[C@H]4C[C@H](n5cnc6c(N)ncnc65)O[C@@H]4COP(=O)(O)O[C@@H]2[C@@H]3F)c(=O)[nH]1] |
| T4-58 | 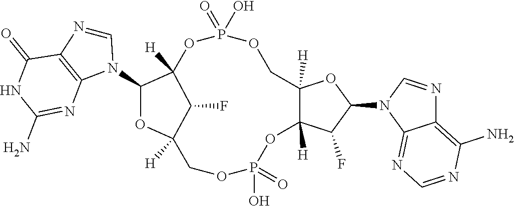 [SMILES: Nc1nc2c(ncn2[C@@H]2O[C@@H]3COP(=O)(O)O[C@H]4[C@@H](F)[C@H](n5cnc6c(N)ncnc65)O[C@@H]4COP(=O)(O)O[C@@H]2[C@@H]3F)c(=O)[nH]1] |
| T4-59 | 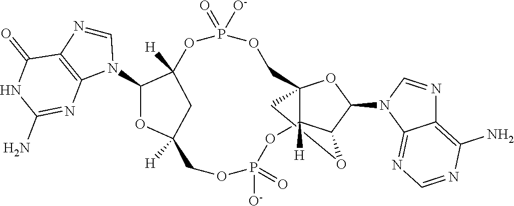 [SMILES: Nc1nc2c(ncn2[C@@H]2O[C@@H]3COP(=O)([O-])O[C@H]4[C@H]5OC[C@]4(COP(=O)([O-])O[C@@H]2C3)O[C@H]5n2cnc3c(N)ncnc32)c(=O)[nH]1] |
| T4-60 | 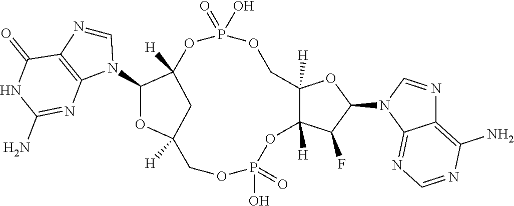 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(=O)(O)O[C@@H]2C3)c(=O)[nH]1] |
| T4-61 | 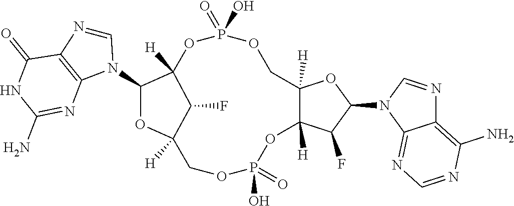 [SMILES: Nc1nc2c(ncn2[C@@H]2O[C@@H]3CO[P@](=O)(O)O[C@H]4[C@H](F)[C@H](n5cnc6c(N)ncnc65)O[C@@H]4CO[P@](=O)(O)O[C@@H]2[C@@H]3F)c(=O)[nH]1] |
| T4-62 | 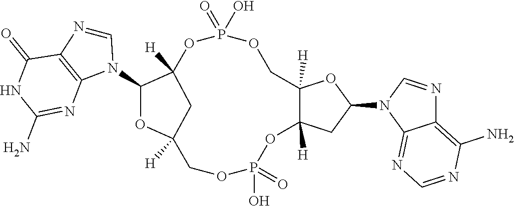 [SMILES: Nc1nc2c(ncn2[C@@H]2O[C@@H]3COP(=O)(O)O[C@H]4C[C@H](n5cnc6c(N)ncnc65)O[C@@H]4COP(=O)(O)O[C@@H]2C3)c(=O)[nH]1] |
| T4-63 | 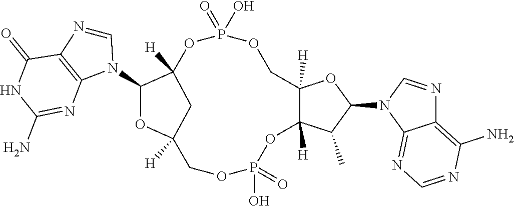 [SMILES: C[C@@H]1[C@@H]2OP(=O)(O)OC[C@@H]3C[C@@H](OP(=O)(O)OC[C@H]2O[C@H]1n1cnc2c(N)ncnc21)[C@H](n1cnc2c(=O)[nH]c(N)nc21)O3] |
| T4-64 | 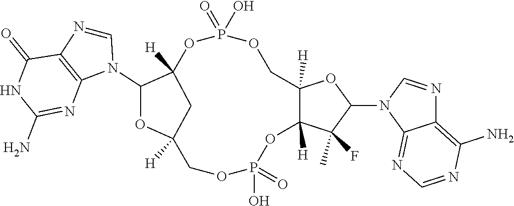 [SMILES: C[C@@]1(F)C(n2cnc3c(N)ncnc32)O[C@@H]2COP(=O)(O)O[C@@H]3C[C@@H](COP(=O)(O)O[C@H]21)OC3n1cnc2c(=O)[nH]c(N)nc21] |
| T4-65 | 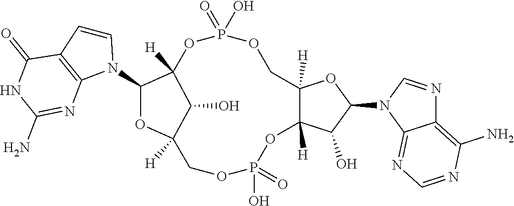 [SMILES: Nc1nc2c(ccn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1] |
| T4-66 | 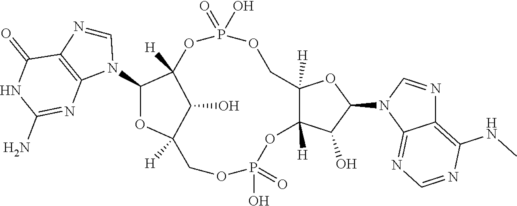 [SMILES: CNc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] |
| T4-67 | 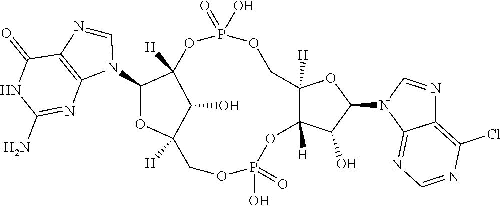 [SMILES: Nc1nc2c(ncn2[C@@H]2O[C@@H]3COP(=O)(O)O[C@H]4[C@@H](O)[C@H](n5cnc6c(Cl)ncnc65)O[C@@H]4COP(=O)(O)O[C@@H]2[C@@H]3O)c(=O)[nH]1] |
| T4-68 | 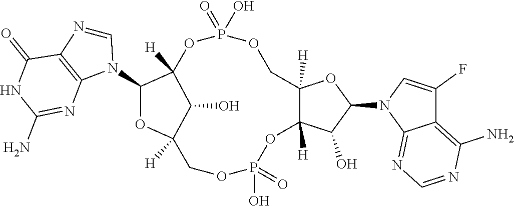 [SMILES: Nc1nc2c(ncn2[C@@H]2O[C@@H]3COP(=O)(O)O[C@H]4[C@@H](O)[C@H](n5cc(F)c6c(N)ncnc65)O[C@@H]4COP(=O)(O)O[C@@H]2[C@@H]3O)c(=O)[nH]1] |
| T4-69 | 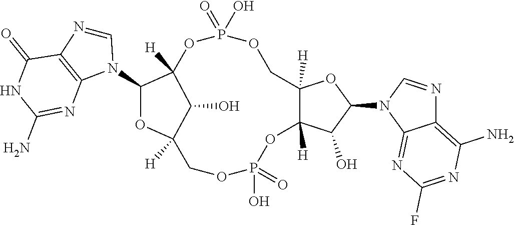 [SMILES: Nc1nc2c(ncn2[C@@H]2O[C@@H]3COP(=O)(O)O[C@H]4[C@@H](O)[C@H](n5cnc6c(N)nc(F)nc65)O[C@@H]4COP(=O)(O)O[C@@H]2[C@@H]3O)c(=O)[nH]1] |
| T4-70 | 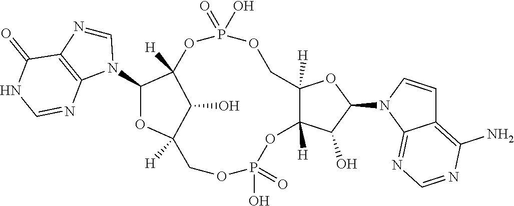 [SMILES: Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]cnc21] |
| T4-71 | 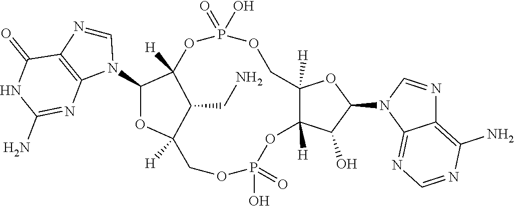 [SMILES: NC[C@H]1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21] |
| T4-72 | 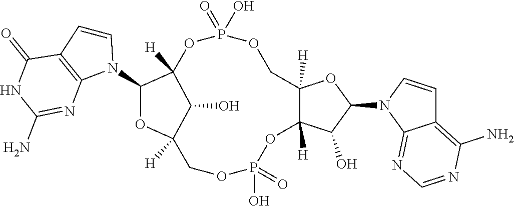 [SMILES: Nc1nc2c(ccn2[C@@H]2O[C@@H]3COP(=O)(O)O[C@H]4[C@@H](O)[C@H](n5ccc6c(N)ncnc65)O[C@@H]4COP(=O)(O)O[C@@H]2[C@@H]3O)c(=O)[nH]1] |
| T4-73 | 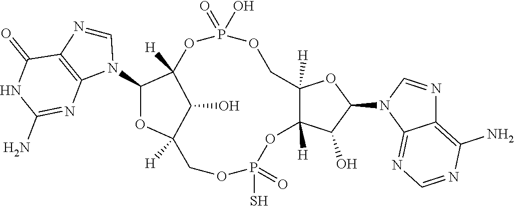 [SMILES: Nc1nc2c(ncn2[C@@H]2O[C@@H]3COP(=O)(S)O[C@H]4[C@@H](O)[C@H](n5cnc6c(N)ncnc65)O[C@@H]4COP(=O)(O)O[C@@H]2[C@@H]3O)c(=O)[nH]1] |
| T4-74 | 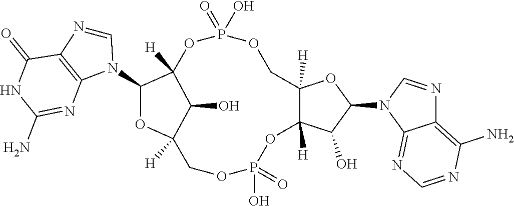 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3O)c(=O)[nH]1] |
| T4-75 | 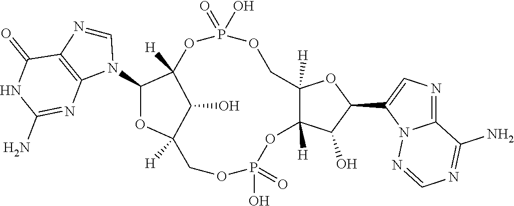 [SMILES: Nc1nc2c(ncn2[C@@H]2O[C@@H]3COP(=O)(O)O[C@H]4[C@@H](O)[C@H](c5cnc6c(N)ncnn56)O[C@@H]4COP(=O)(O)O[C@@H]2[C@@H]3O)c(=O)[nH]1] |
| T4-76 | 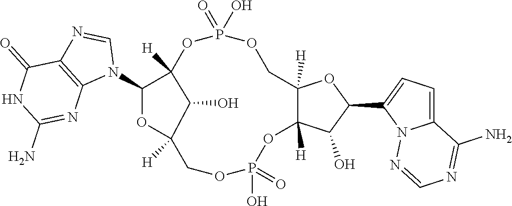 [SMILES: Nc1nc2c(ncn2[C@@H]2O[C@@H]3COP(=O)(O)O[C@H]4[C@@H](O)[C@H](c5ccc6c(N)ncnn56)O[C@@H]4COP(=O)(O)O[C@@H]2[C@@H]3O)c(=O)[nH]1] |
| T4-77 | 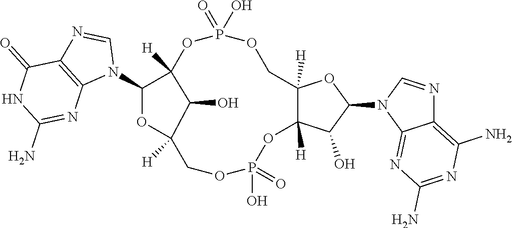 [SMILES: Nc1nc(N)c2ncn([C@@H]3O[C@@H]4COP(=O)(O)O[C@@H]5[C@@H](O)[C@@H](COP(=O)(O)O[C@H]4[C@H]3O)O[C@H]5n3cnc4c(=O)[nH]c(N)nc43)c2n1] |
| T4-78 | 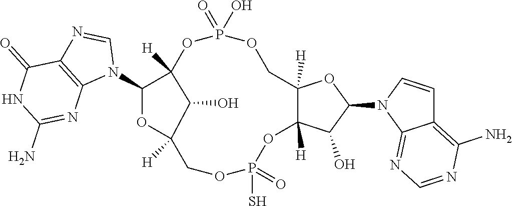 [SMILES: Nc1nc2c(ncn2[C@@H]2O[C@@H]3COP(=O)(S)O[C@H]4[C@@H](O)[C@H](n5ccc6c(N)ncnc65)O[C@@H]4COP(=O)(O)O[C@@H]2[C@@H]3O)c(=O)[nH]1] |
| T4-79 | 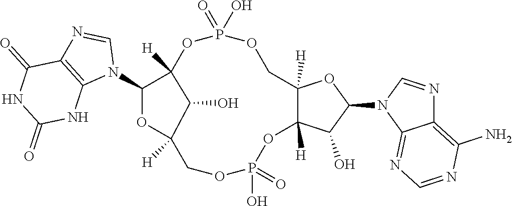 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(=O)[nH]c21] |
| T4-80 | 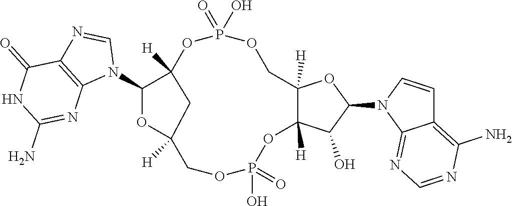 [SMILES: Nc1nc2c(ncn2[C@@H]2O[C@@H]3COP(=O)(O)O[C@H]4[C@@H](O)[C@H](n5ccc6c(N)ncnc65)O[C@@H]4COP(=O)(O)O[C@@H]2C3)c(=O)[nH]1] |
| T4-81 | 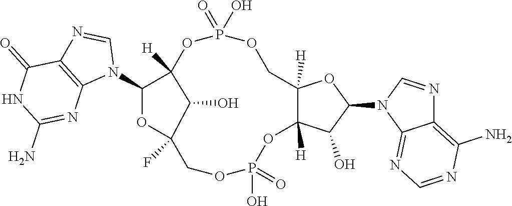 [SMILES: Nc1nc2c(ncn2[C@@H]2O[C@]3(F)COP(=O)(O)O[C@H]4[C@@H](O)[C@H](n5cnc6c(N)ncnc65)O[C@@H]4COP(=O)(O)O[C@@H]2[C@@H]3O)c(=O)[nH]1] |
| T4-82 | 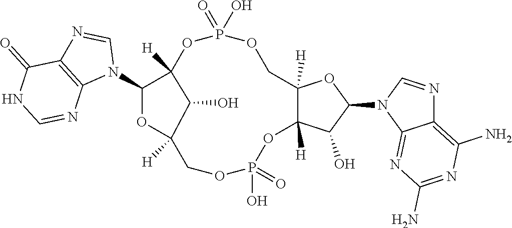 [SMILES: Nc1nc(N)c2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]cnc43)c2n1] |
| T4-83 | 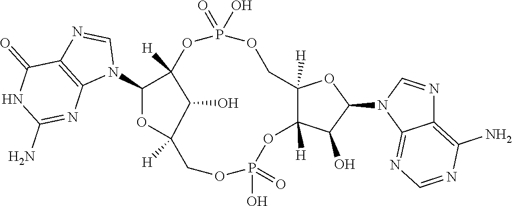 [SMILES: Nc1nc2c(ncn2[C@@H]2O[C@@H]3COP(=O)(O)O[C@H]4[C@H](O)[C@H](n5cnc6c(N)ncnc65)O[C@@H]4COP(=O)(O)O[C@@H]2[C@@H]3O)c(=O)[nH]1] |
| T4-84 | 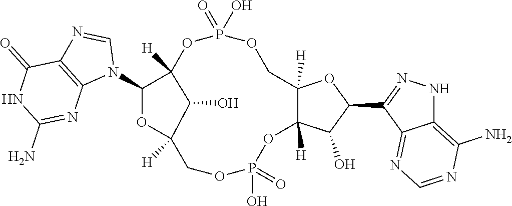 [SMILES: Nc1nc2c(ncn2[C@@H]2O[C@@H]3COP(=O)(O)O[C@H]4[C@@H](O)[C@H](c5n[nH]c6c(N)ncnc56)O[C@@H]4COP(=O)(O)O[C@@H]2[C@@H]3O)c(=O)[nH]1] |
| T4-85 | 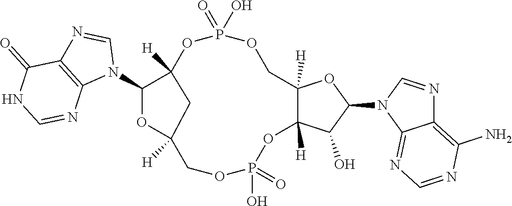 [SMILES: Nc1ncnc2c1ncn2[C@@H]1O[C@@H]2COP(=O)(O)O[C@@H]3C[C@@H](COP(=O)(O)O[C@H]2[C@H]1O)O[C@H]3n1cnc2c(=O)[nH]cnc21] |
| T4-86 | 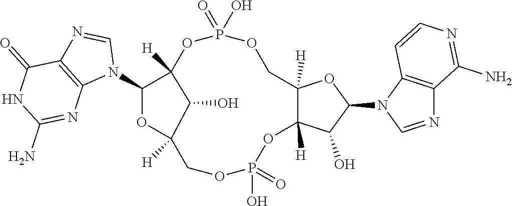 [SMILES: Nc1nc2c(ncn2[C@@H]2O[C@@H]3COP(=O)(O)O[C@H]4[C@@H](O)[C@H](n5cnc6c(N)nccc65)O[C@@H]4COP(=O)(O)O[C@@H]2[C@@H]3O)c(=O)[nH]1] |
| T4-87 | 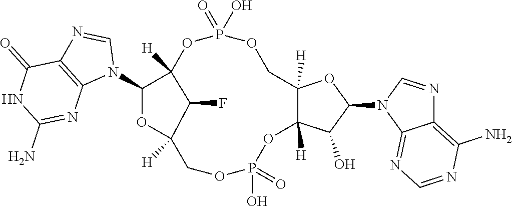 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@H]3F)c(=O)[nH]1] |
| T4-88 | 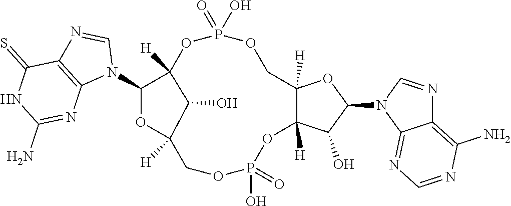 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=S)[nH]1] |
| T4-89 | 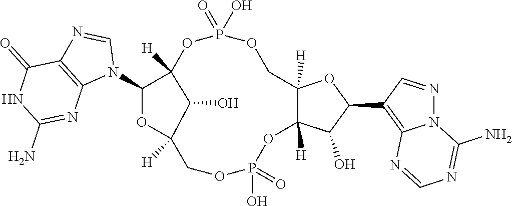 [SMILES: Nc1nc2c(ncn2[C@@H]2O[C@@H]3COP(=O)(O)O[C@H]4[C@@H](O)[C@H](c5cnn6c(N)ncnc56)O[C@@H]4COP(=O)(O)O[C@@H]2[C@@H]3O)c(=O)[nH]1] |
| T4-90 | 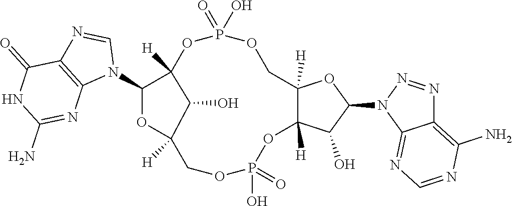 [SMILES: Nc1nc2c(ncn2[C@@H]2O[C@@H]3COP(=O)(O)O[C@H]4[C@@H](O)[C@H](n5nnc6c(N)ncnc65)O[C@@H]4COP(=O)(O)O[C@@H]2[C@@H]3O)c(=O)[nH]1] |
| T4-91 | 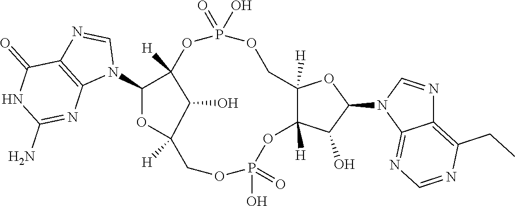 [SMILES: CCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] |
| T4-92 | 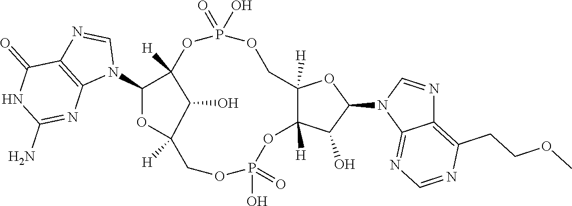 [SMILES: COCCc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] |
| T4-93 | 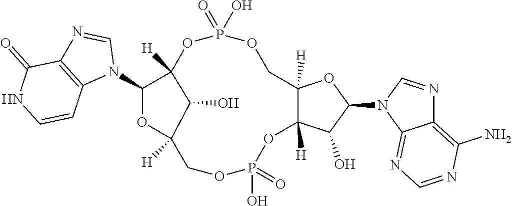 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21] |
| T4-94 | 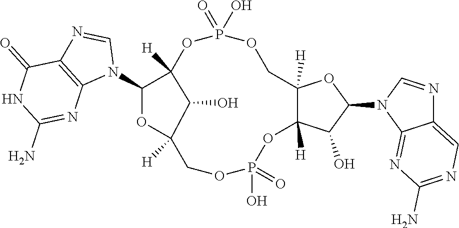 [SMILES: Nc1ncc2ncn([C@@H]3O[C@@H]4COP(=O)(O)O[C@@H]5[C@H](O)[C@@H](COP(=O)(O)O[C@H]4[C@H]3O)O[C@H]5n3cnc4c(=O)[nH]c(N)nc43)c2n1] |
| T4-95 | 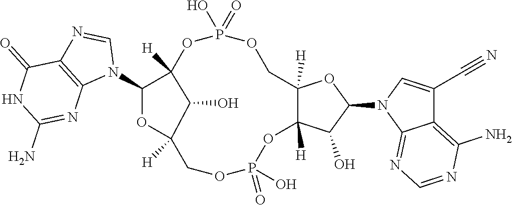 [SMILES: N#Cc1cn([C@@H]2O[C@@H]3COP(=O)(O)O[C@@H]4[C@H](O)[C@@H](COP(=O)(O)O[C@H]3[C@H]2O)O[C@H]4n2cnc3c(=O)[nH]c(N)nc32)c2ncnc(N)c12] |
| T4-96 | 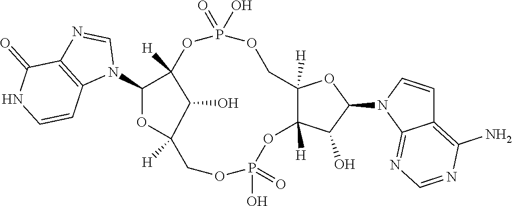 [SMILES: Nc1ncnc2c1ccn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ccc21] |
| T4-97 | 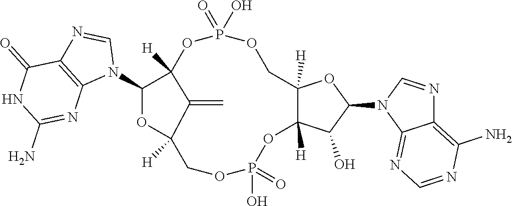 [SMILES: C=C1[C@H]2OP(=O)(O)OC[C@H]3O[C@@H](n4cnc5c(N)ncnc54)[C@H](O)[C@@H]3OP(=O)(O)OC[C@H]1O[C@H]2n1cnc2c(=O)[nH]c(N)nc21] |
| T4-98 | 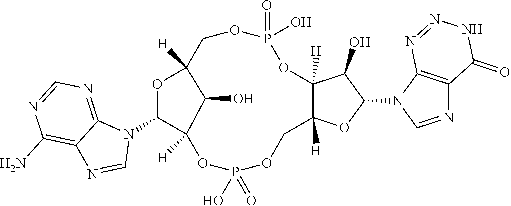 [SMILES: Nc1ncnc2c1ncn2[C@@H]1O[C@@H]2COP(=O)(O)O[C@H]3[C@@H](O)[C@H](n4cnc5c(=O)[nH]nnc54)O[C@@H]3COP(=O)(O)O[C@@H]1[C@@H]2O] |
| T4-99 | 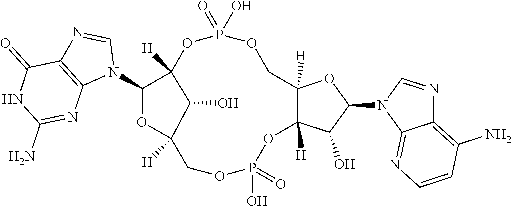 [SMILES: Nc1nc2c(ncn2[C@@H]2O[C@@H]3COP(=O)(O)O[C@H]4[C@@H](O)[C@H](n5cnc6c(N)ccnc65)O[C@@H]4COP(=O)(O)O[C@@H]2[C@@H]3O)c(=O)[nH]1] |
| T4-100 | 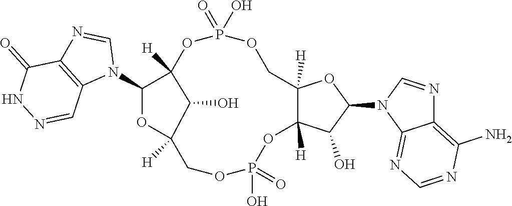 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]ncc21] |
| T4-101 | 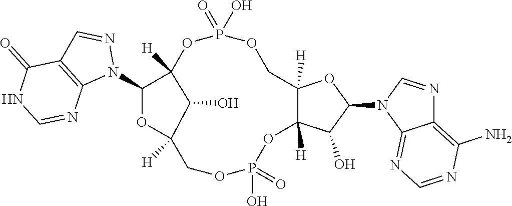 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1ncc2c(=O)[nH]cnc21] |
| T4-102 | 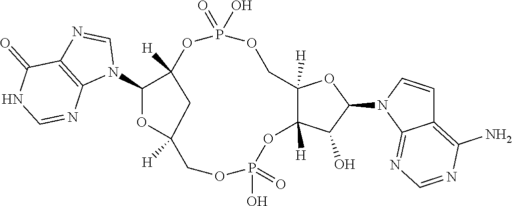 [SMILES: Nc1ncnc2c1ccn2[C@@H]1O[C@@H]2COP(=O)(O)O[C@@H]3C[C@@H](COP(=O)(O)O[C@H]2[C@H]1O)O[C@H]3n1cnc2c(=O)[nH]cnc21] |
| T4-103 | 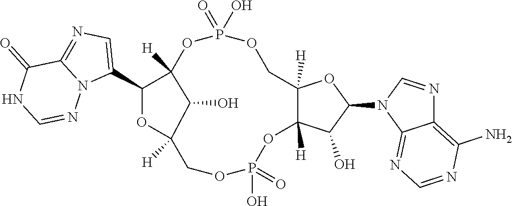 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1cnc2c(=O)[nH]cnn12] |
| T4-104 | 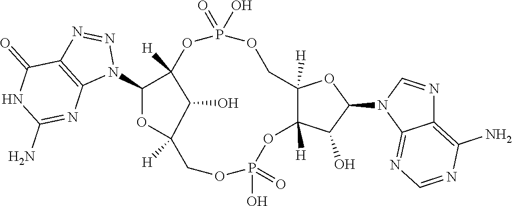 [SMILES: Nc1nc2c(nnn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3O)c(=O)[nH]1] |
| T4-105 | 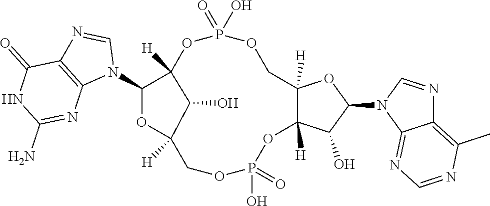 [SMILES: Cc1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] |
| T4-106 | 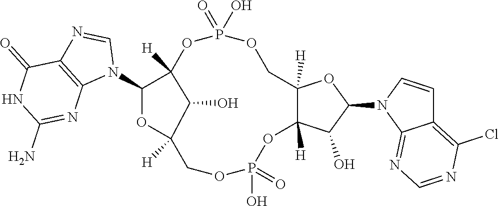 [SMILES: Nc1nc2c(ncn2[C@@H]2O[C@@H]3COP(=O)(O)O[C@H]4[C@@H](O)[C@H](n5ccc6c(Cl)ncnc65)O[C@@H]4COP(=O)(O)O[C@@H]2[C@@H]3O)c(=O)[nH]1] |
| T4-107 | 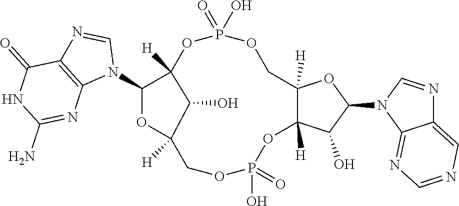 [SMILES: Nc1nc2c(ncn2[C@@H]2O[C@@H]3COP(=O)(O)O[C@H]4[C@@H](O)[C@H](n5cnc6cncnc65)O[C@@H]4COP(=O)(O)O[C@@H]2[C@@H]3O)c(=O)[nH]1] |
| T4-108 | 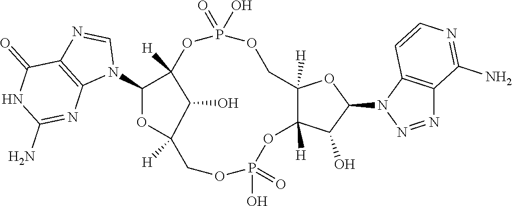 [SMILES: Nc1nc2c(ncn2[C@@H]2O[C@@H]3COP(=O)(O)O[C@H]4[C@@H](O)[C@H](n5nnc6c(N)nccc65)O[C@@H]4COP(=O)(O)O[C@@H]2[C@@H]3O)c(=O)[nH]1] |
| T4-109 | 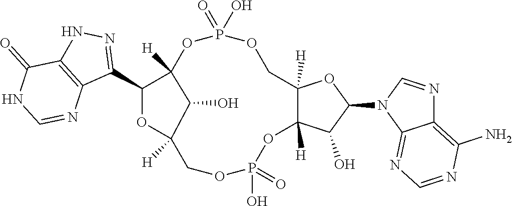 [SMILES: Nc1ncnc2c1ncn2[C@@H]1O[C@@H]2COP(=O)(O)O[C@@H]3[C@H](O)[C@@H](COP(=O)(O)O[C@H]2[C@H]1O)O[C@H]3c1n[nH]c2c(=O)[nH]cnc12] |
| T4-110 | 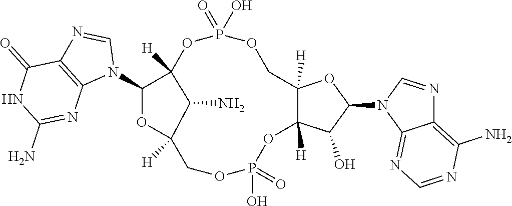 [SMILES: Nc1nc2c(ncn2[C@@H]2O[C@@H]3COP(=O)(O)O[C@H]4[C@@H](O)[C@H](n5cnc6c(N)ncnc65)O[C@@H]4COP(=O)(O)O[C@@H]2[C@@H]3N)c(=O)[nH]1] |
| T4-111 | 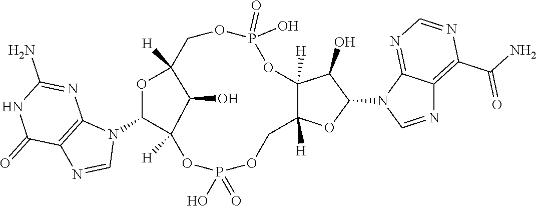 [SMILES: NC(=O)c1ncnc2c1ncn2[C@@H]1O[C@@H]2COP(=O)(O)O[C@@H]3[C@H](O)[C@@H](COP(=O)(O)O[C@H]2[C@H]1O)O[C@H]3n1cnc2c(=O)[nH]c(N)nc21] |
| T4-112 | 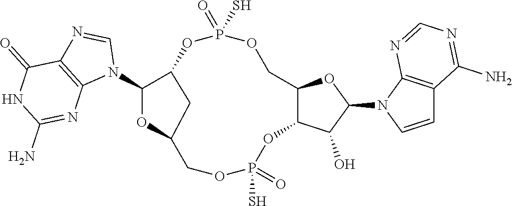 [SMILES: Nc1nc2c(ncn2[C@@H]2O[C@@H]3CO[P@@](=O)(S)O[C@H]4[C@@H](O)[C@H](n5ccc6c(N)ncnc65)O[C@@H]4CO[P@@](=O)(S)O[C@@H]2C3)c(=O)[nH]1] |
| T4-113 | 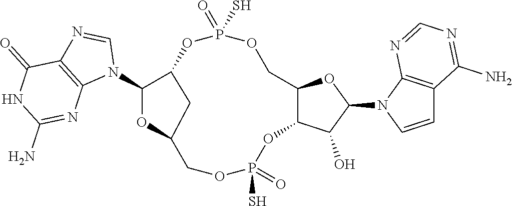 [SMILES: Nc1nc2c(ncn2[C@@H]2O[C@@H]3CO[P@](=O)(S)O[C@H]4[C@@H](O)[C@H](n5ccc6c(N)ncnc65)O[C@@H]4CO[P@@](=O)(S)O[C@@H]2C3)c(=O)[nH]1] |
| T4-114 | 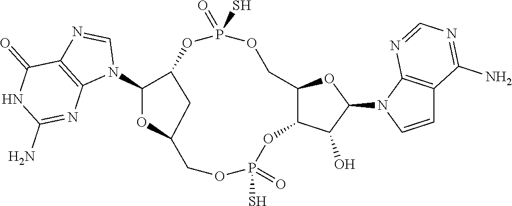 [SMILES: Nc1nc2c(ncn2[C@@H]2O[C@@H]3CO[P@@](=O)(S)O[C@H]4[C@@H](O)[C@H](n5ccc6c(N)ncnc65)O[C@@H]4CO[P@](=O)(S)O[C@@H]2C3)c(=O)[nH]1] |
| T4-115 | 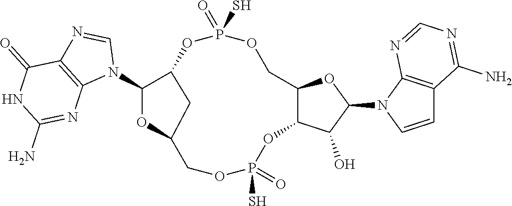 [SMILES: Nc1nc2c(ncn2[C@@H]2O[C@@H]3CO[P@](=O)(S)O[C@H]4[C@@H](O)[C@H](n5ccc6c(N)ncnc65)O[C@@H]4CO[P@](=O)(S)O[C@@H]2C3)c(=O)[nH]1] |
| T4-116 | 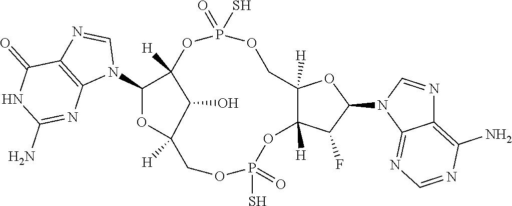 [SMILES: Nc1nc2c(ncn2[C@@H]2O[C@@H]3COP(=O)(S)O[C@H]4[C@@H](F)[C@H](n5cnc6c(N)ncnc65)O[C@@H]4COP(=O)(S)O[C@@H]2[C@@H]3O)c(=O)[nH]1] |
| T4-117 | 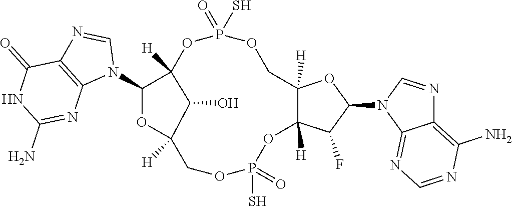 [SMILES: Nc1nc2c(ncn2[C@@H]2O[C@@H]3COP(=O)(S)O[C@H]4[C@@H](F)[C@H](n5cnc6c(N)ncnc65)O[C@@H]4COP(=O)(S)O[C@@H]2[C@@H]3O)c(=O)[nH]1] |
| T4-118 | 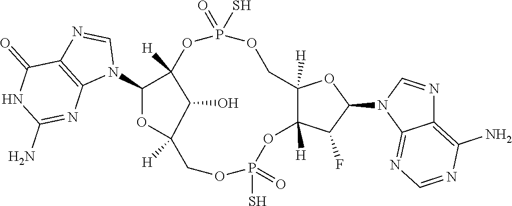 [SMILES: Nc1nc2c(ncn2[C@@H]2O[C@@H]3COP(=O)(S)O[C@H]4[C@@H](F)[C@H](n5cnc6c(N)ncnc65)O[C@@H]4COP(=O)(S)O[C@@H]2[C@@H]3O)c(=O)[nH]1] |
| T4-119 | 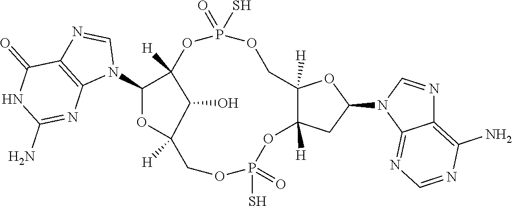 [SMILES: Nc1nc2c(ncn2[C@@H]2O[C@@H]3COP(=O)(S)O[C@H]4C[C@H](n5cnc6c(N)ncnc65)O[C@@H]4COP(=O)(S)O[C@@H]2[C@@H]3O)c(=O)[nH]1] |
| T4-120 | 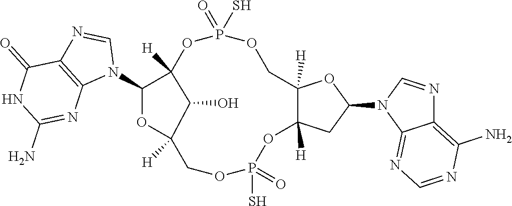 [SMILES: Nc1nc2c(ncn2[C@@H]2O[C@@H]3COP(=O)(S)O[C@H]4C[C@H](n5cnc6c(N)ncnc65)O[C@@H]4COP(=O)(S)O[C@@H]2[C@@H]3O)c(=O)[nH]1] |
| T4-121 | 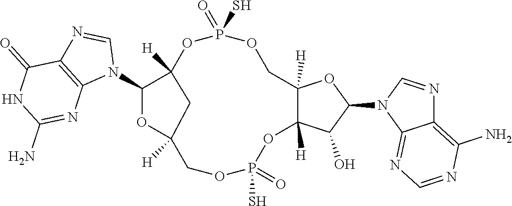 [SMILES: Nc1nc2c(ncn2[C@@H]2O[C@@H]3CO[P@@](=O)(S)O[C@H]4[C@@H](O)[C@H](n5cnc6c(N)ncnc65)O[C@@H]4CO[P@](=O)(S)O[C@@H]2C3)c(=O)[nH]1] |
| T4-122 | 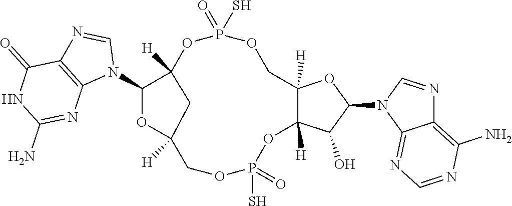 [SMILES: Nc1nc2c(ncn2[C@@H]2O[C@@H]3COP(=O)(S)O[C@H]4[C@@H](O)[C@H](n5cnc6c(N)ncnc65)O[C@@H]4COP(=O)(S)O[C@@H]2C3)c(=O)[nH]1] |
| T4-123 | 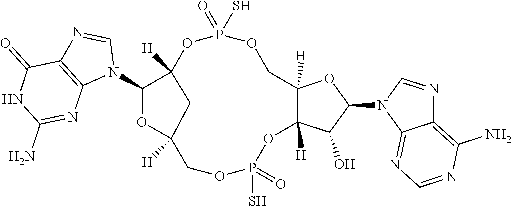 [SMILES: Nc1nc2c(ncn2[C@@H]2O[C@@H]3COP(=O)(S)O[C@H]4[C@@H](O)[C@H](n5cnc6c(N)ncnc65)O[C@@H]4COP(=O)(S)O[C@@H]2C3)c(=O)[nH]1] |
| T4-124 | 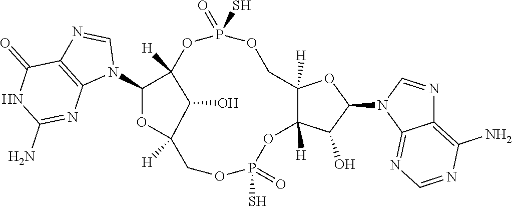 [SMILES: Nc1nc2c(ncn2[C@@H]2O[C@@H]3CO[P@@](=O)(S)O[C@H]4[C@@H](O)[C@H](n5cnc6c(N)ncnc65)O[C@@H]4CO[P@](=O)(S)O[C@@H]2[C@@H]3O)c(=O)[nH]1] |
| T4-125 | 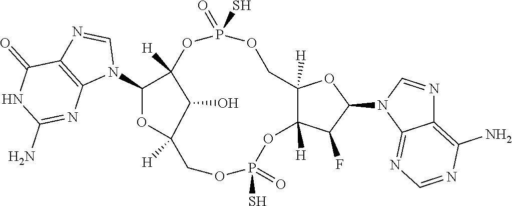 [SMILES: Nc1nc2c(ncn2[C@@H]2O[C@@H]3CO[P@](=O)(S)O[C@H]4[C@H](F)[C@H](n5cnc6c(N)ncnc65)O[C@@H]4CO[P@](=O)(S)O[C@@H]2[C@@H]3O)c(=O)[nH]1] |
| T4-126 | 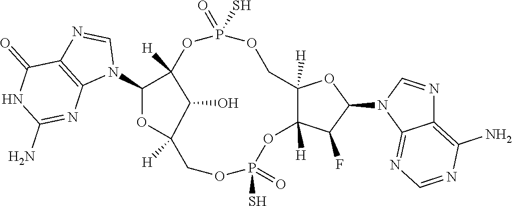 [SMILES: Nc1nc2c(ncn2[C@@H]2O[C@@H]3CO[P@](=O)(S)O[C@H]4[C@H](F)[C@H](n5cnc6c(N)ncnc65)O[C@@H]4CO[P@@](=O)(S)O[C@@H]2[C@@H]3O)c(=O)[nH]1] |
| T4-127 | 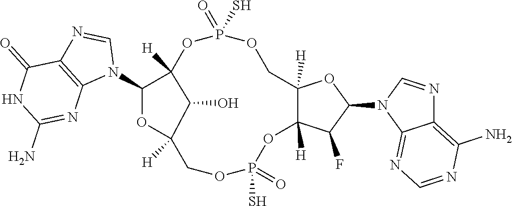 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3O)c(=O)[nH]1] |
| T4-128 | 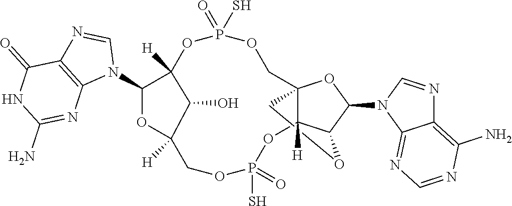 [SMILES: Nc1nc2c(ncn2[C@@H]2O[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-129 | 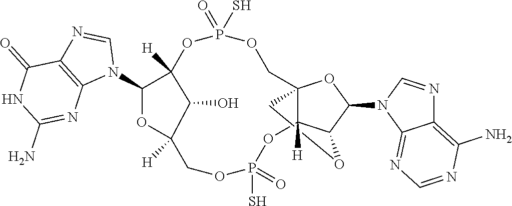 [SMILES: Nc1nc2c(ncn2[C@@H]2O[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-130 | 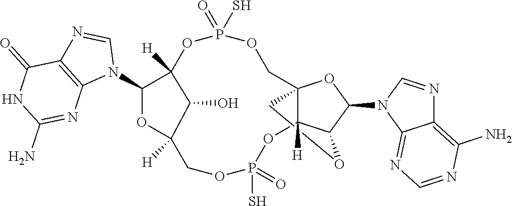 [SMILES: Nc1nc2c(ncn2[C@@H]2O[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-131 | 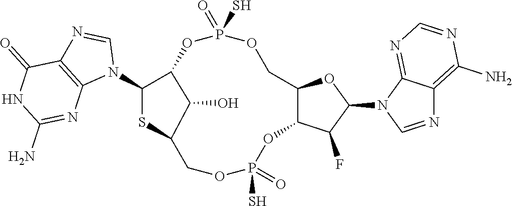 [SMILES: Nc1nc2c(ncn2[C@@H]2S[C@@H]3CO[P@](=O)(S)O[C@H]4[C@H](F)[C@H](n5cnc6c(N)ncnc65)O[C@@H]4CO[P@](=O)(S)O[C@@H]2[C@@H]3O)c(=O)[nH]1] |
| T4-132 | 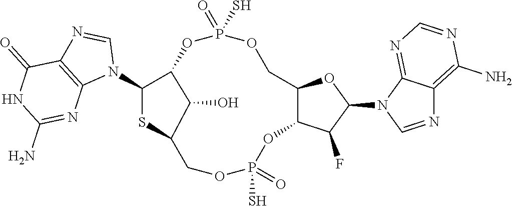 [SMILES: Nc1nc2c(ncn2[C@@H]2S[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3O)c(=O)[nH]1] |
| T4-133 | 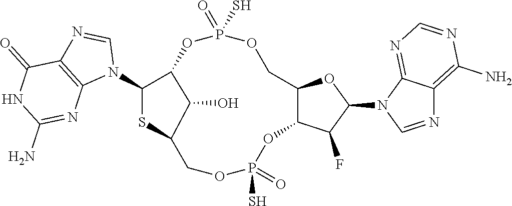 [SMILES: Nc1nc2c(ncn2[C@@H]2S[C@@H]3CO[P@](=O)(S)O[C@H]4[C@H](F)[C@H](n5cnc6c(N)ncnc65)O[C@@H]4CO[P@@](=O)(S)O[C@@H]2[C@@H]3O)c(=O)[nH]1] |
| T4-134 | 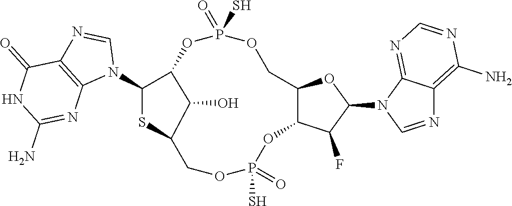 [SMILES: Nc1nc2c(ncn2[C@@H]2S[C@@H]3CO[P@@](=O)(S)O[C@H]4[C@H](F)[C@H](n5cnc6c(N)ncnc65)O[C@@H]4CO[P@](=O)(S)O[C@@H]2[C@@H]3O)c(=O)[nH]1] |
| T4-135 | 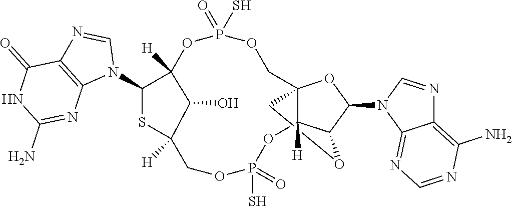 [SMILES: Nc1nc2c(ncn2[C@@H]2S[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-136 | 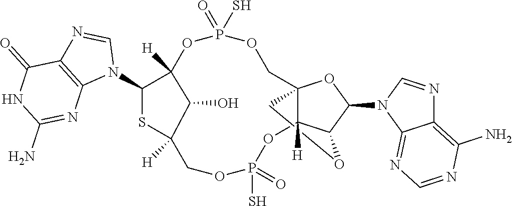 [SMILES: Nc1nc2c(ncn2[C@@H]2S[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-137 | 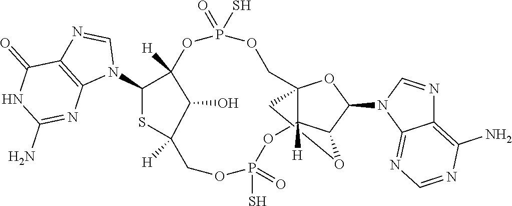 [SMILES: Nc1nc2c(ncn2[C@@H]2S[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-138 | 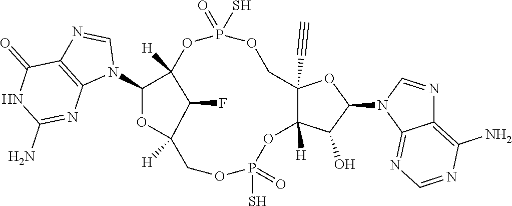 [SMILES: C#C[C@@]12COP(=O)(S)O[C@@H]3[C@@H](F)[C@@H](COP(=O)(S)O[C@H]1[C@@H](O)[C@H](n1cnc4c(N)ncnc41)O2)O[C@H]3n1cnc2c(=O)[nH]c(N)nc21] |
| T4-139 | 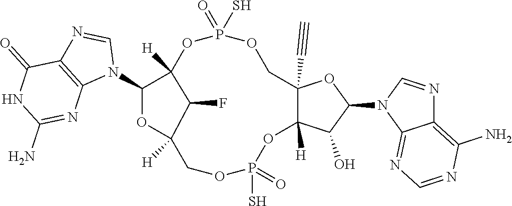 [SMILES: C#C[C@@]12COP(=O)(S)O[C@@H]3[C@@H](F)[C@@H](COP(=O)(S)O[C@H]1[C@@H](O)[C@H](n1cnc4c(N)ncnc41)O2)O[C@H]3n1cnc2c(=O)[nH]c(N)nc21] |
| T4-140 | 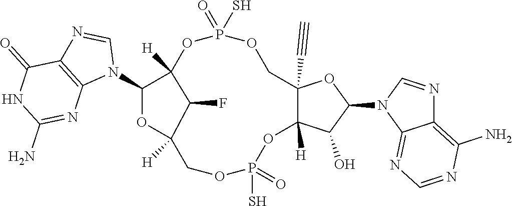 [SMILES: C#C[C@@]12COP(=O)(S)O[C@@H]3[C@@H](F)[C@@H](COP(=O)(S)O[C@H]1[C@@H](O)[C@H](n1cnc4c(N)ncnc41)O2)O[C@H]3n1cnc2c(=O)[nH]c(N)nc21] |
| T4-141 | 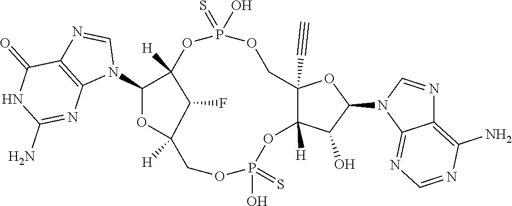 [SMILES: C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21] |
| T4-142 | 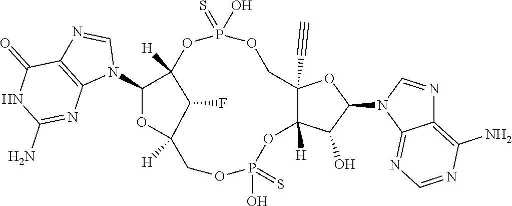 [SMILES: C#C[C@@]12COP(O)(=S)O[C@@H]3[C@H](F)[C@@H](COP(O)(=S)O[C@H]1[C@@H](O)[C@H](n1cnc4c(N)ncnc41)O2)O[C@H]3n1cnc2c(=O)[nH]c(N)nc21] |
| T4-143 | 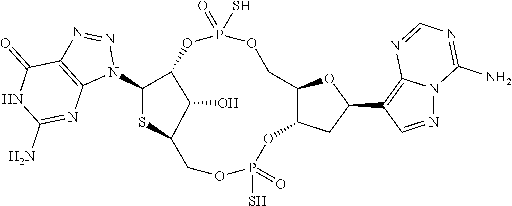 [SMILES: Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1] |
| T4-144 | 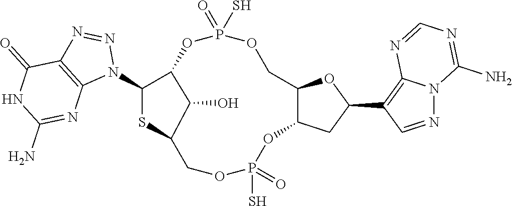 [SMILES: Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1] |
| T4-145 | 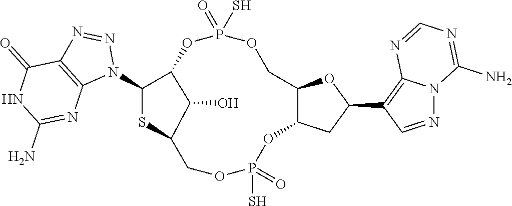 [SMILES: Nc1nc2c(nnn2[C@@H]2S[C@@H]3COP(=O)(S)O[C@H]4C[C@H](c5cnn6c(N)ncnc56)O[C@@H]4COP(=O)(S)O[C@@H]2[C@@H]3O)c(=O)[nH]1] |
| T4-146 | 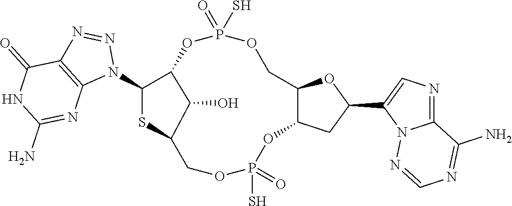 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1] |
| T4-147 | 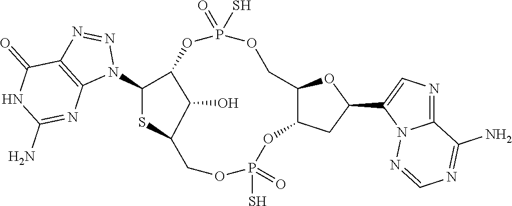 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1] |
| T4-148 | 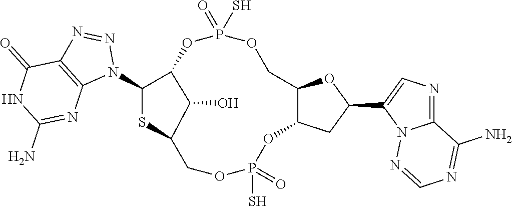 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1] |
| T4-149 | 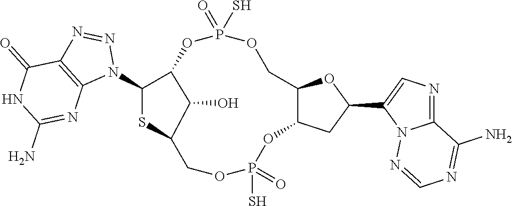 [SMILES: Nc1nc2c(nnn2[C@@H]2S[C@@H]3COP(=O)(S)O[C@H]4C[C@H](c5cnc6c(N)ncnn56)O[C@@H]4COP(=O)(S)O[C@@H]2[C@@H]3O)c(=O)[nH]1] |
| T4-150 | 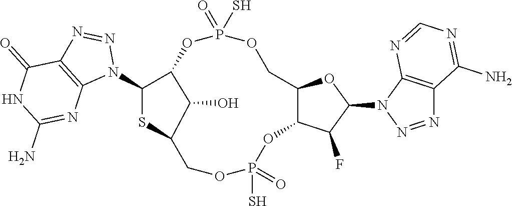 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1] |
| T4-151 | 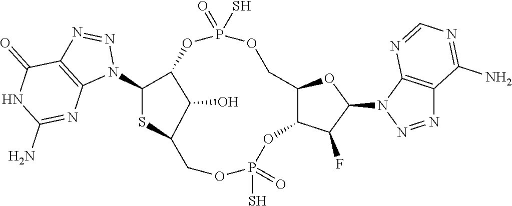 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3O)c(=O)[nH]1] |
| T4-152 | 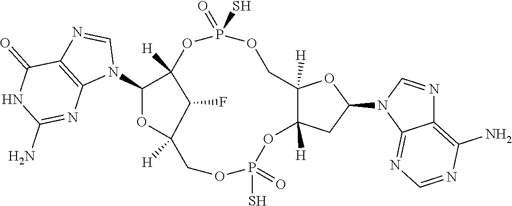 [SMILES: Nc1nc2c(ncn2[C@@H]2O[C@@H]3COP(=O)(S)O[C@H]4C[C@H](n5cnc6c(N)ncnc65)O[C@@H]4CO[P@](=O)(S)O[C@@H]2[C@@H]3F)c(=O)[nH]1] |
| T4-153 | 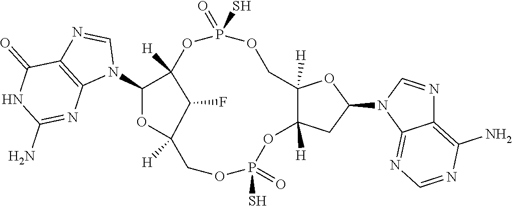 [SMILES: Nc1nc2c(ncn2[C@@H]2O[C@@H]3CO[P@](=O)(S)O[C@H]4C[C@H](n5cnc6c(N)ncnc65)O[C@@H]4CO[P@](=O)(S)O[C@@H]2[C@@H]3F)c(=O)[nH]1] |
| T4-154 | 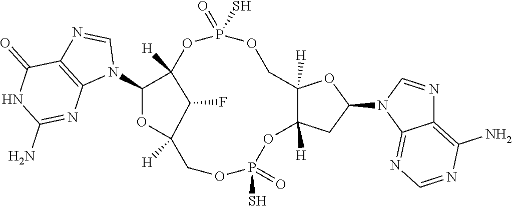 [SMILES: Nc1nc2c(ncn2[C@@H]2O[C@@H]3CO[P@](=O)(S)O[C@H]4C[C@H](n5cnc6c(N)ncnc65)O[C@@H]4CO[P@@](=O)(S)O[C@@H]2[C@@H]3F)c(=O)[nH]1] |
| T4-155 | 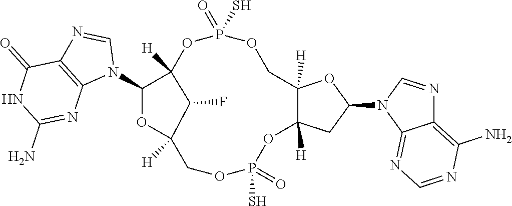 [SMILES: Nc1nc2c(ncn2[C@@H]2O[C@@H]3CO[P@@](=O)(S)O[C@H]4C[C@H](n5cnc6c(N)ncnc65)O[C@@H]4CO[P@@](=O)(S)O[C@@H]2[C@@H]3F)c(=O)[nH]1] |
| T4-156 | 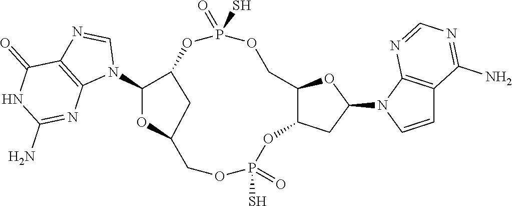 [SMILES: Nc1nc2c(ncn2[C@@H]2O[C@@H]3CO[P@@](=O)(S)O[C@H]4C[C@H](n5ccc6c(N)ncnc65)O[C@@H]4CO[P@](=O)(S)O[C@@H]2C3)c(=O)[nH]1] |
| | di |
| T4-157 | 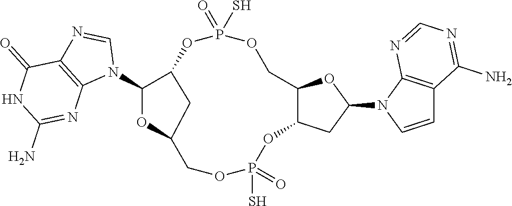 [SMILES: Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2C3)c(=O)[nH]1] |
| T4-158 | 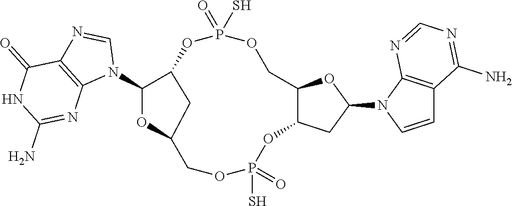 [SMILES: Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2C3)c(=O)[nH]1] |
| T4-159 | 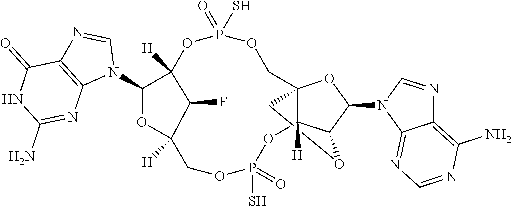 [SMILES: Nc1nc2c(ncn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-160 | 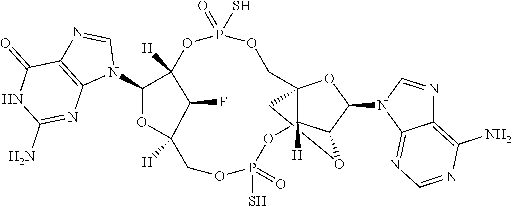 [SMILES: Nc1nc2c(ncn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-161 | 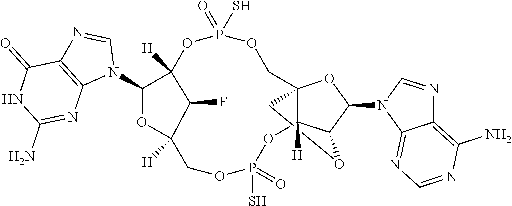 [SMILES: Nc1nc2c(ncn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-162 | 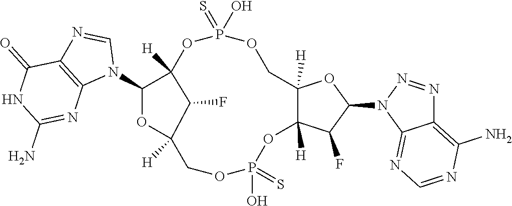 [SMILES: Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1] |
| T4-163 | 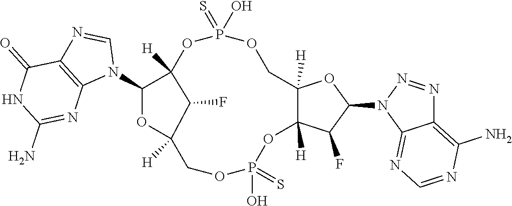 [SMILES: Nc1nc2c(ncn2[C@@H]2O[C@@H]3COP(O)(=S)O[C@H]4[C@H](F)[C@H](n5nnc6c(N)ncnc65)O[C@@H]4COP(O)(=S)O[C@@H]2[C@@H]3F)c(=O)[nH]1] |
| T4-164 | 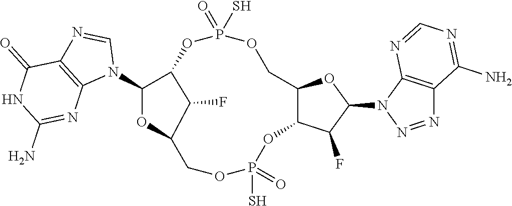 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-165 | 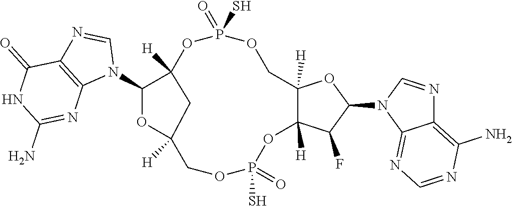 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@](=O)(S)O[C@@H]2C3)c(=O)[nH]1] |
| T4-166 | 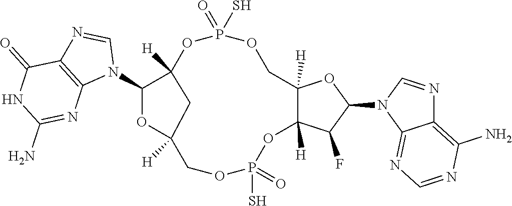 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2C3)c(=O)[nH]1] |
| T4-167 | 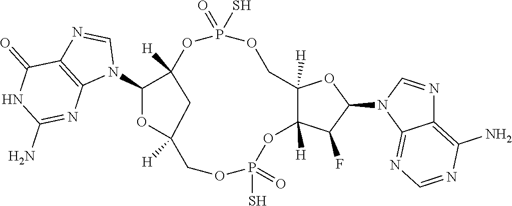 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2C3)c(=O)[nH]1] |
| T4-168 | 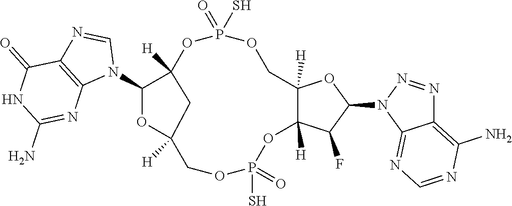 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1] |
| T4-169 | 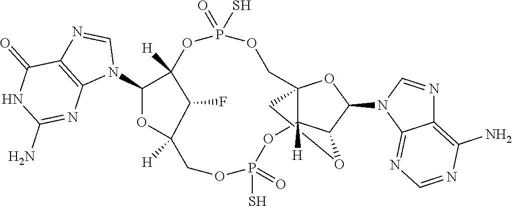 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1] |
| T4-170 | 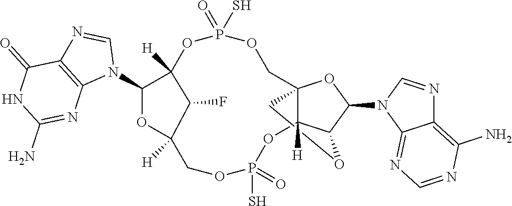 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1] |
| T4-171 | 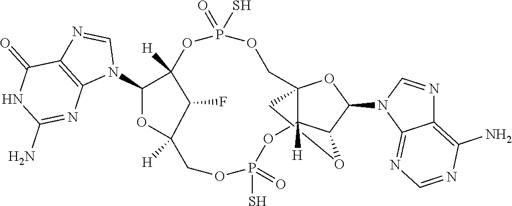 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1] |
| T4-172 | 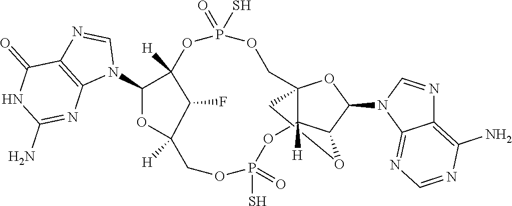 [SMILES: Nc1nc2c(ncn2[C@@H]2O[C@@H]3COP(=O)(S)O[C@H]4[C@H]5OC[C@]4(COP(=O)(S)O[C@@H]2[C@@H]3F)O[C@H]5n2cnc3c(N)ncnc32)c(=O)[nH]1] |
| T4-173 | 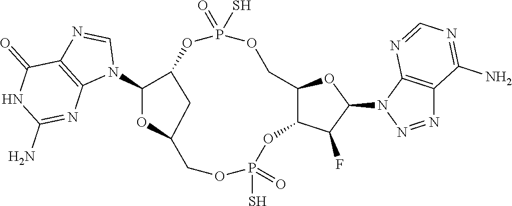 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1] |
| T4-174 | 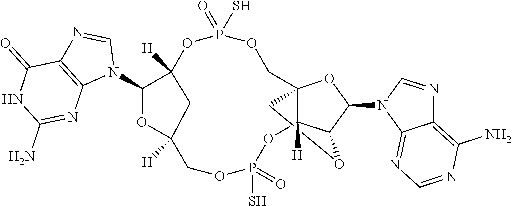 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1] |
| T4-175 | 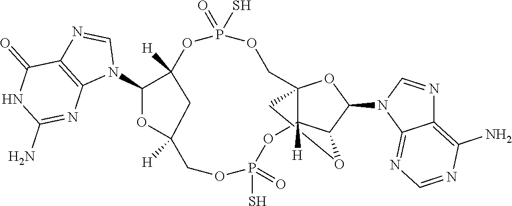 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1] |
| T4-176 | 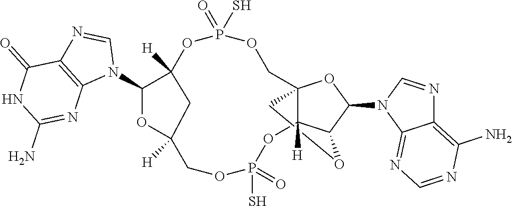 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1] |
| T4-177 | 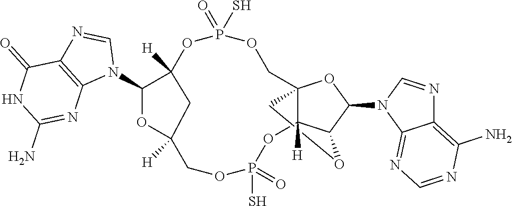 [SMILES: Nc1nc2c(ncn2[C@@H]2O[C@@H]3COP(=O)(S)O[C@H]4[C@H]5OC[C@]4(COP(=O)(S)O[C@@H]2C3)O[C@H]5n2cnc3c(N)ncnc32)c(=O)[nH]1] |
| T4-178 | 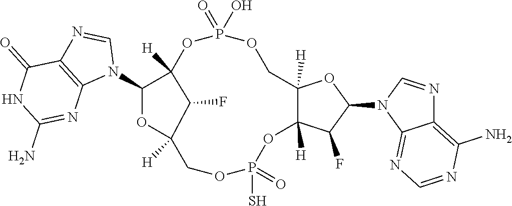 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(O)O[C@@H]2[C@@H]3F)c(=O)[nH]1] |
| T4-179 | 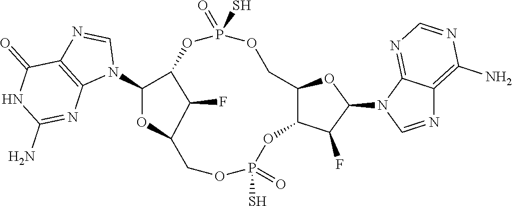 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@](=O)(S)O[C@@H]2[C@H]3F)c(=O)[nH]1] |
| T4-180 | 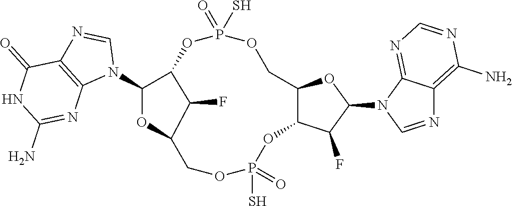 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1] |
| T4-181 | 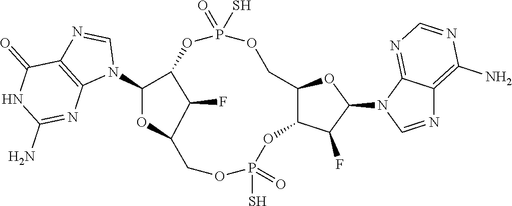 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1] |
| T4-182 | 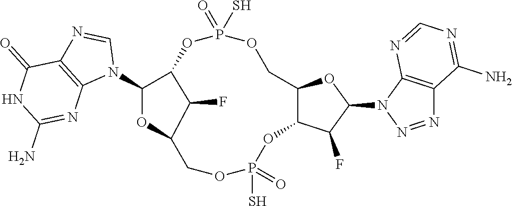 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] |
| T4-183 | 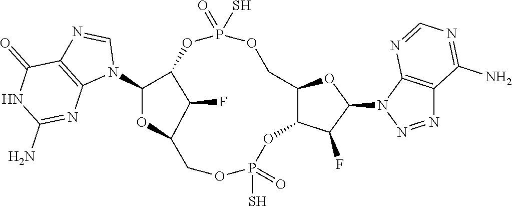 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] |
| T4-184 | 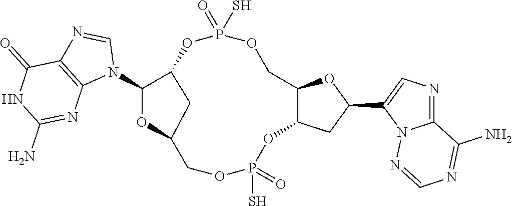 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1] |
| T4-185 | 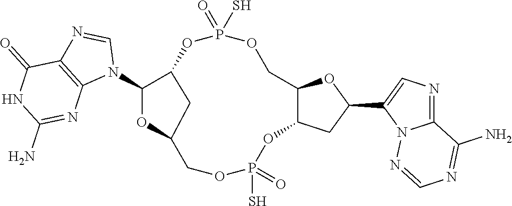 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2C3)c(=O)[nH]1] |
| T4-186 | 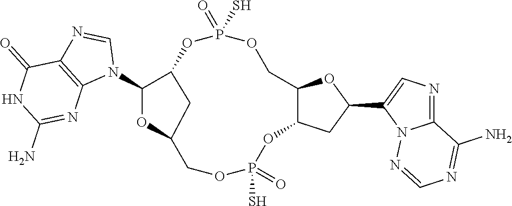 [SMILES: Nc1nc2c(ncn2[C@@H]2O[C@@H]3CO[P@@](=O)(S)O[C@H]4C[C@H](c5cnc6c(N)ncnn56)O[C@@H]4CO[P@@](=O)(S)O[C@@H]2C3)c(=O)[nH]1] |
| T4-187 | 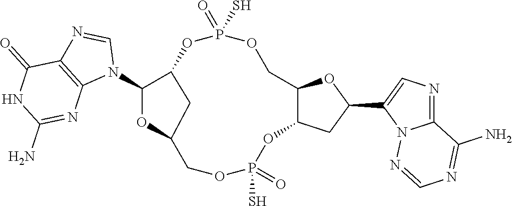 [SMILES: Nc1nc2c(ncn2[C@@H]2O[C@@H]3CO[P@@](=O)(S)O[C@H]4C[C@H](c5cnc6c(N)ncnn56)O[C@@H]4CO[P@@](=O)(S)O[C@@H]2C3)c(=O)[nH]1] |
| T4-188 | 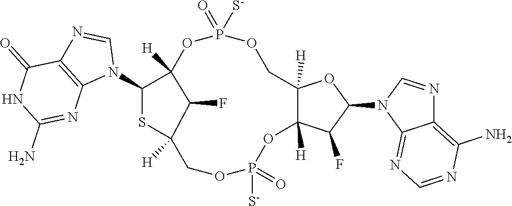 [SMILES: Nc1nc2c(ncn2[C@@H]2S[C@@H]3COP(=O)([S-])O[C@H]4[C@H](F)[C@H](n5cnc6c(N)ncnc65)O[C@@H]4COP(=O)([S-])O[C@@H]2[C@H]3F)c(=O)[nH]1] |
| T4-189 | 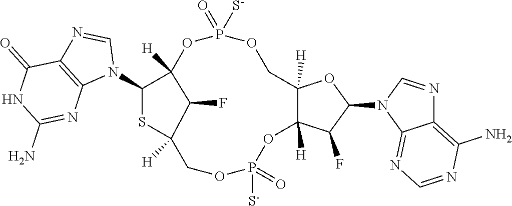 [SMILES: Nc1nc2c(ncn2[C@@H]2S[C@@H]3COP(=O)([S-])O[C@H]4[C@H](F)[C@H](n5cnc6c(N)ncnc65)O[C@@H]4COP(=O)([S-])O[C@@H]2[C@H]3F)c(=O)[nH]1] |
| T4-190 | 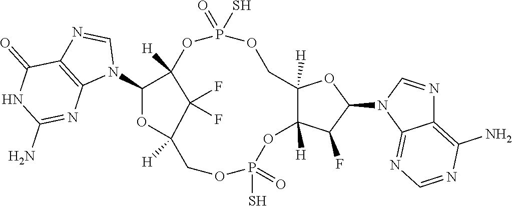 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2C3(F)F)c(=O)[nH]1] |
| T4-191 | 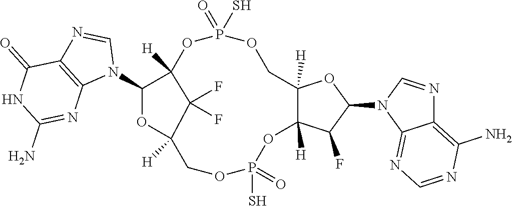 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2C3(F)F)c(=O)[nH]1] |
| T4-192 | 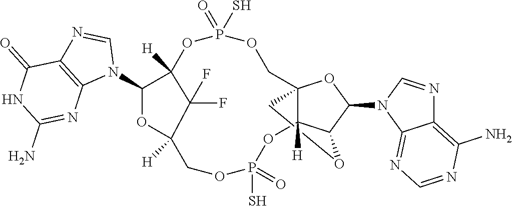 [SMILES: Nc1nc2c(ncn2[C@@H]2O[C@@H]3COP(=O)(S)O[C@H]4[C@H]5OC[C@]4(COP(=O)(S)O[C@@H]2C3(F)F)O[C@H]5n2cnc3c(N)ncnc32)c(=O)[nH]1] |
| T4-193 | 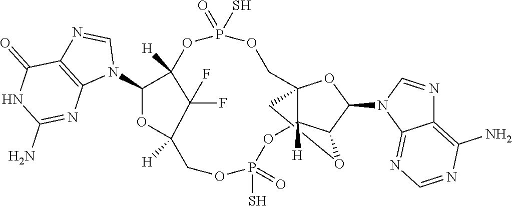 [SMILES: Nc1nc2c(ncn2[C@@H]2O[C@@H]3COP(=O)(S)O[C@H]4[C@H]5OC[C@]4(COP(=O)(S)O[C@@H]2C3(F)F)O[C@H]5n2cnc3c(N)ncnc32)c(=O)[nH]1] |
| T4-194 | 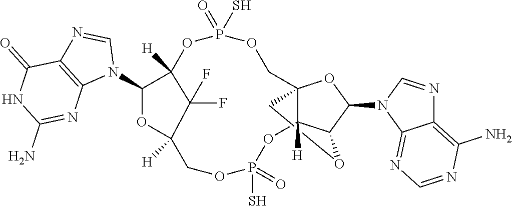 [SMILES: Nc1nc2c(ncn2[C@@H]2O[C@@H]3COP(=O)(S)O[C@H]4[C@H]5OC[C@]4(COP(=O)(S)O[C@@H]2C3(F)F)O[C@H]5n2cnc3c(N)ncnc32)c(=O)[nH]1] |
| T4-195 | 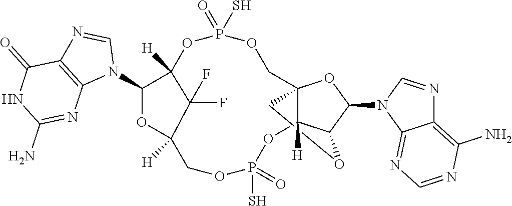 [SMILES: Nc1nc2c(ncn2[C@@H]2O[C@@H]3COP(=O)(S)O[C@H]4[C@H]5OC[C@]4(COP(=O)(S)O[C@@H]2C3(F)F)O[C@H]5n2cnc3c(N)ncnc32)c(=O)[nH]1] |
| T4-196 | 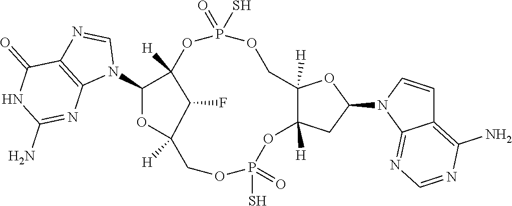 [SMILES: Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2[C@@H]3F)c(=O)[nH]1] |
| T4-197 | 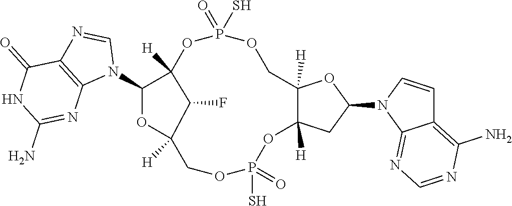 [SMILES: Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2[C@@H]3F)c(=O)[nH]1] |
| T4-198 | 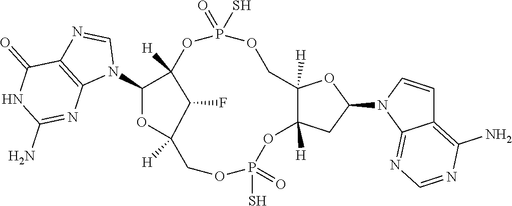 [SMILES: Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2[C@@H]3F)c(=O)[nH]1] |
| T4-199 | 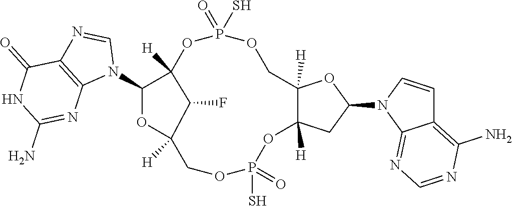 [SMILES: Nc1nc2c(ncn2[C@@H]2O[C@@H]3COP(=O)(S)O[C@H]4C[C@H](n5ccc6c(N)ncnc65)O[C@@H]4COP(=O)(S)O[C@@H]2[C@@H]3F)c(=O)[nH]1] |
| T4-200 | 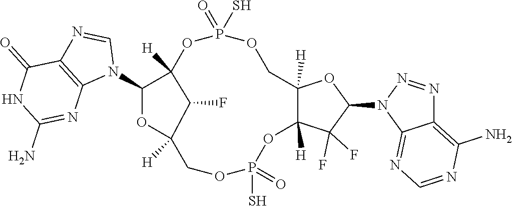 [SMILES: Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(N)ncnc32)C4(F)F)c(=O)[nH]1] |
| T4-201 | 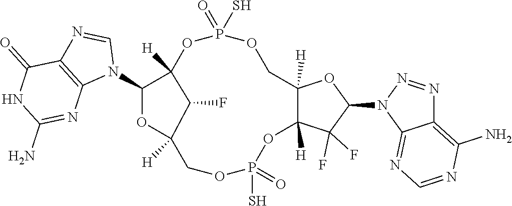 [SMILES: Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(N)ncnc32)C4(F)F)c(=O)[nH]1] |
| T4-202 | 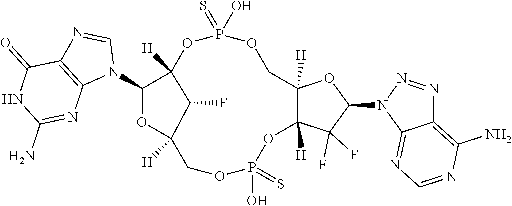 [SMILES: Nc1nc2c(ncn2[C@@H]2O[C@@H]3COP(O)(=S)O[C@@H]4[C@@H](COP(O)(=S)O[C@@H]2[C@@H]3F)O[C@@H](n2nnc3c(N)ncnc32)C4(F)F)c(=O)[nH]1] |
| T4-203 | 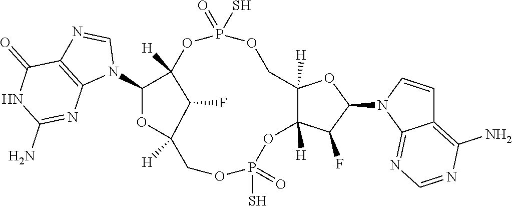 [SMILES: Nc1nc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1] |
| T4-204 | 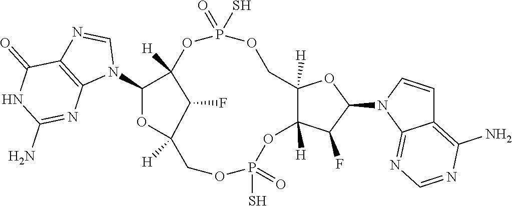 [SMILES: Nc1nc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1] |
| T4-205 | 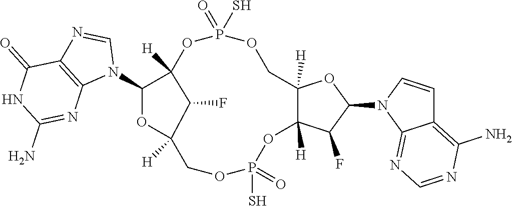 [SMILES: Nc1nc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1] |
| T4-206 | 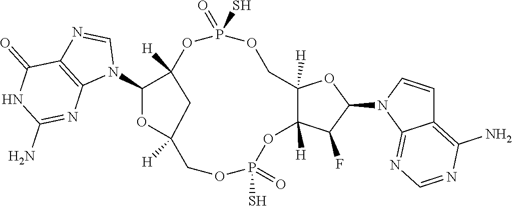 [SMILES: Nc1nc2c(ncn2[C@@H]2O[C@@H]3CO[P@@](=O)(S)O[C@H]4[C@H](F)[C@H](n5ccc6c(N)ncnc65)O[C@@H]4CO[P@](=O)(S)O[C@@H]2C3)c(=O)[nH]1] |
| T4-207 | 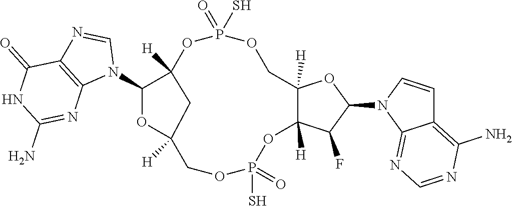 [SMILES: Nc1nc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2C3)c(=O)[nH]1] |
| T4-208 | 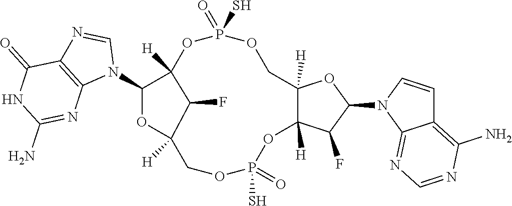 [SMILES: Nc1nc2c(ncn2[C@@H]2O[C@@H]3CO[P@@](=O)(S)O[C@H]4[C@H](F)[C@H](n5ccc6c(N)ncnc65)O[C@@H]4CO[P@](=O)(S)O[C@@H]2[C@H]3F)c(=O)[nH]1] |
| T4-209 | 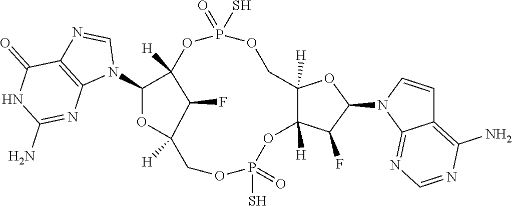 [SMILES: Nc1nc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@H]3F)c(=O)[nH]1] |
| T4-210 | 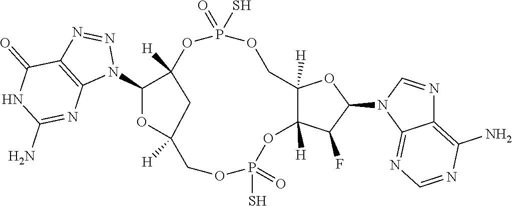 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2C3)c(=O)[nH]1] |
| T4-211 | 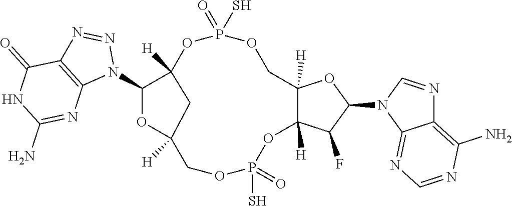 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2C3)c(=O)[nH]1] |
| T4-212 | 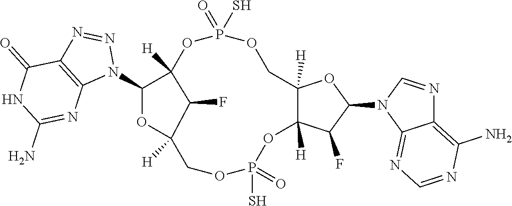 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1] |
| T4-213 | 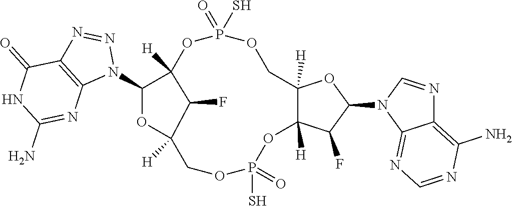 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1] |
| T4-214 | 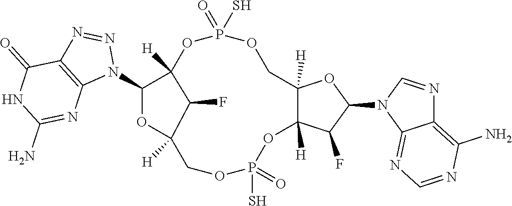 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2[C@H]3F)c(=O)[nH]1] |
| T4-215 | 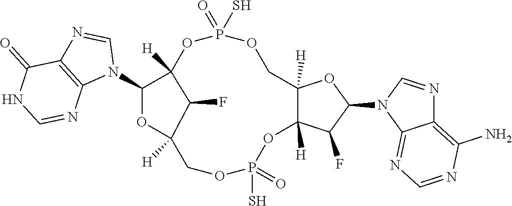 [SMILES: Nc1ncnc2c1ncn2[C@@H]1O[C@@H]2COP(=O)(S)O[C@@H]3[C@@H](F)[C@@H](COP(=O)(S)O[C@H]2[C@@H]1F)O[C@H]3n1cnc2c(=O)[nH]cnc21] |
| T4-216 | 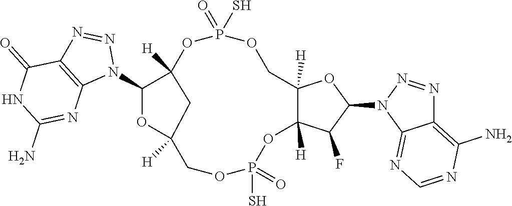 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1] |
| T4-217 | 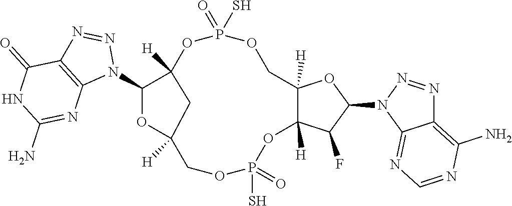 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1] |
| T4-218 | 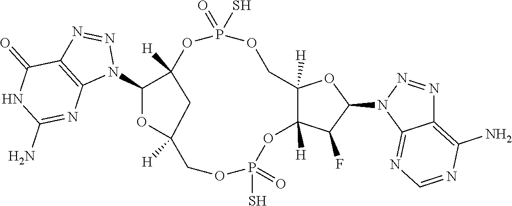 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@@H]2C3)c(=O)[nH]1] |
| T4-219 | 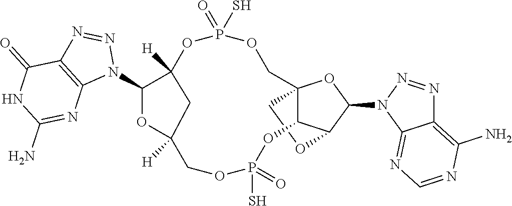 [SMILES: Nc1nc2c(nnn2[C@@H]2O[C@@H]3COP(=O)(S)O[C@H]4[C@H]5OC[C@]4(COP(=O)(S)O[C@@H]2C3)O[C@H]5n2nnc3c(N)ncnc32)c(=O)[nH]1] |
| T4-220 | 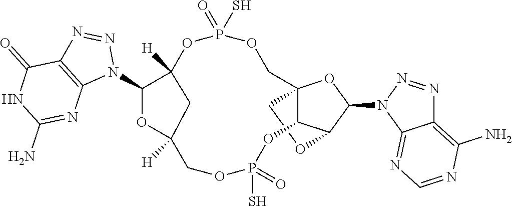 [SMILES: Nc1nc2c(nnn2[C@@H]2O[C@@H]3COP(=O)(S)O[C@H]4[C@H]5OC[C@]4(COP(=O)(S)O[C@@H]2C3)O[C@H]5n2nnc3c(N)ncnc32)c(=O)[nH]1] |
| T4-221 | 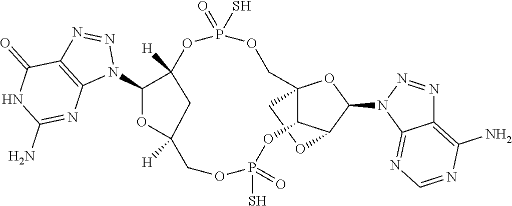 [SMILES: Nc1nc2c(nnn2[C@@H]2O[C@@H]3COP(=O)(S)O[C@H]4[C@H]5OC[C@]4(COP(=O)(S)O[C@@H]2C3)O[C@H]5n2nnc3c(N)ncnc32)c(=O)[nH]1] |
| T4-222 | 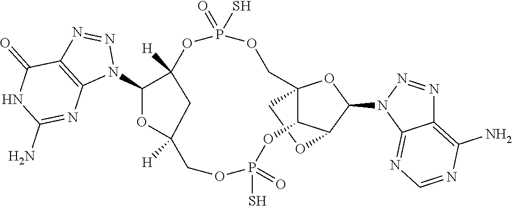 [SMILES: Nc1nc2c(nnn2[C@@H]2O[C@@H]3COP(=O)(S)O[C@H]4[C@H]5OC[C@]4(COP(=O)(S)O[C@@H]2C3)O[C@H]5n2nnc3c(N)ncnc32)c(=O)[nH]1] |
| T4-223 T1-40 | 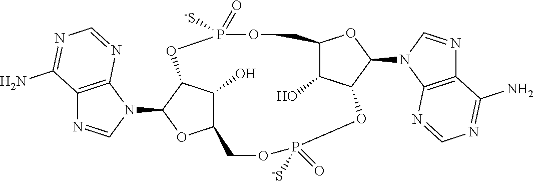 |
| T4-224 T1-41 | 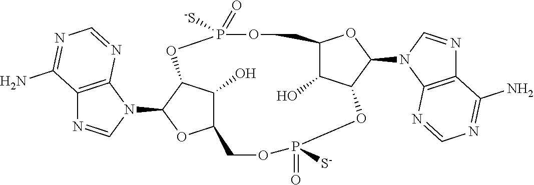 |
| T4-225 | 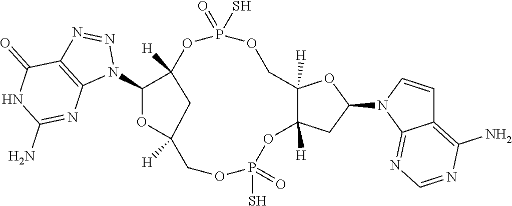 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@@H]2C3)c(=O)[nH]1] |
| T4-226 | 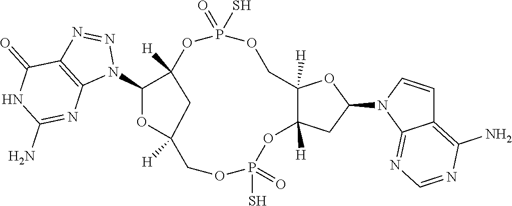 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@@H]2C3)c(=O)[nH]1] |
| T4-227 | 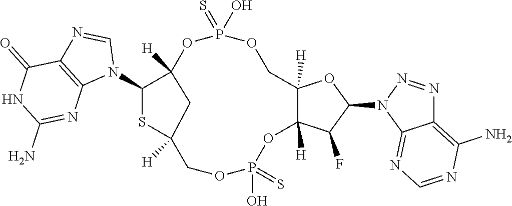 [SMILES: Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1] |
| T4-228 | 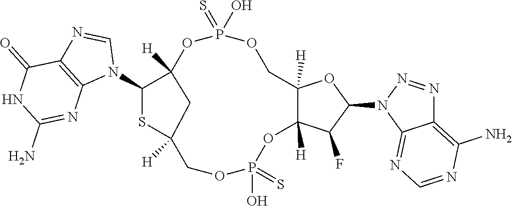 [SMILES: Nc1nc2c(ncn2[C@@H]2S[C@@H]3COP(O)(=S)O[C@H]4[C@H](F)[C@H](n5nnc6c(N)ncnc65)O[C@@H]4COP(O)(=S)O[C@@H]2C3)c(=O)[nH]1] |
| T4-229 | 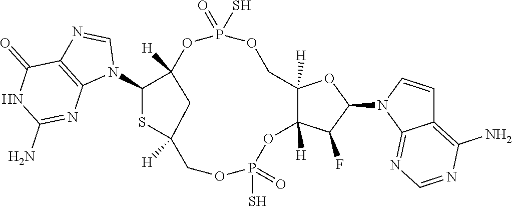 [SMILES: Nc1nc2c(ncn2[C@@H]2S[C@@H]3COP(=O)(S)O[C@H]4[C@H](F)[C@H](n5ccc6c(N)ncnc65)O[C@@H]4COP(=O)(S)O[C@@H]2C3)c(=O)[nH]1] |
| T4-230 | 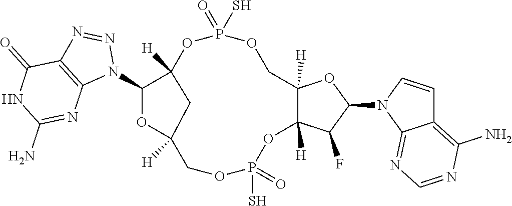 [SMILES: Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2C3)c(=O)[nH]1] |
| T4-231 | 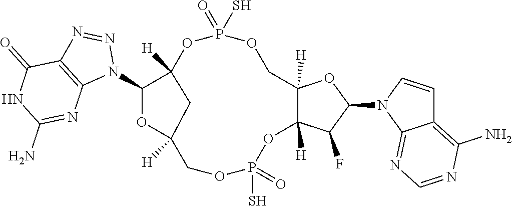 [SMILES: Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2C3)c(=O)[nH]1] |
| T4-232 | 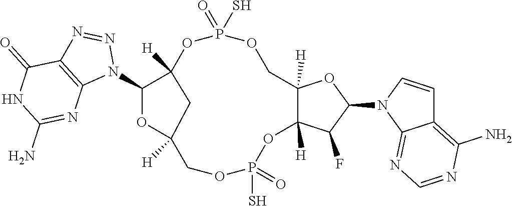 [SMILES: Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2C3)c(=O)[nH]1] |
| T4-233 | 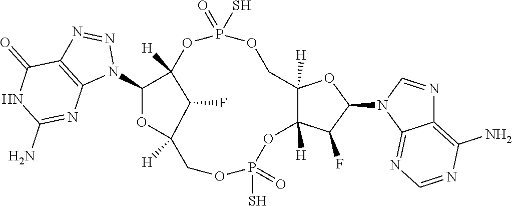 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-234 | 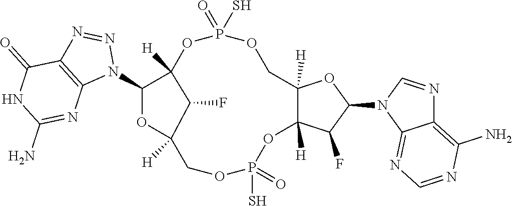 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-235 | 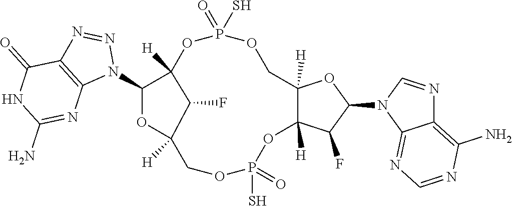 [SMILES: Nc1nc2c(nnn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-236 | 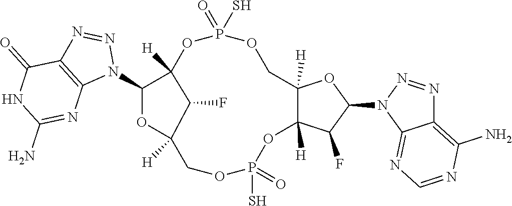 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-237 | 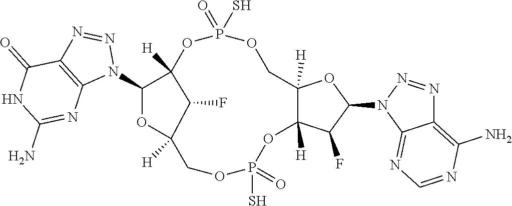 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-238 | 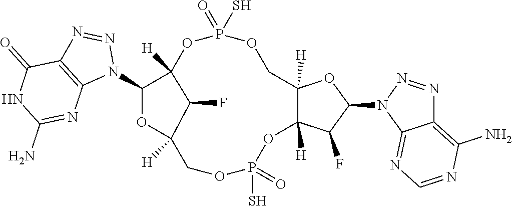 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] |
| T4-239 | 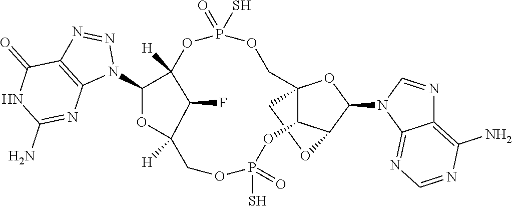 [SMILES: Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-240 | 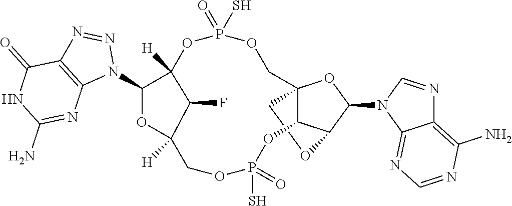 [SMILES: Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-241 | 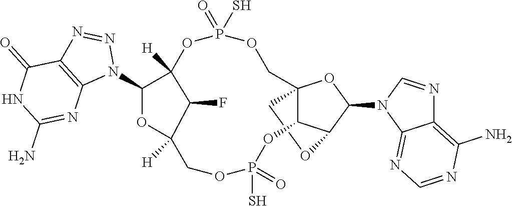 [SMILES: Nc1nc2c(nnn2[C@@H]2O[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-242 | 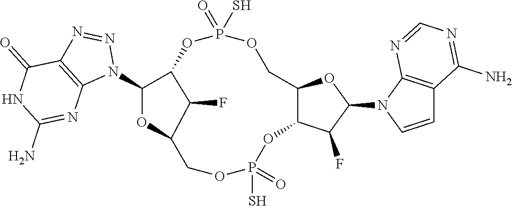 [SMILES: Nc1nc2c(nnn2[C@@H]2O[C@@H]3COP(=O)(S)O[C@H]4[C@H](F)[C@H](n5ccc6c(N)ncnc65)O[C@@H]4COP(=O)(S)O[C@@H]2[C@H]3F)c(=O)[nH]1] |
| T4-243 | 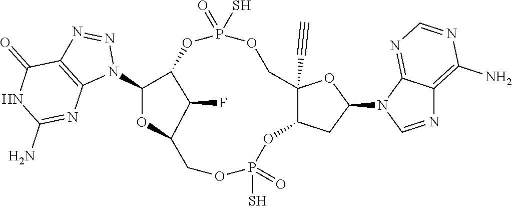 [SMILES: C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21] |
| T4-244 | 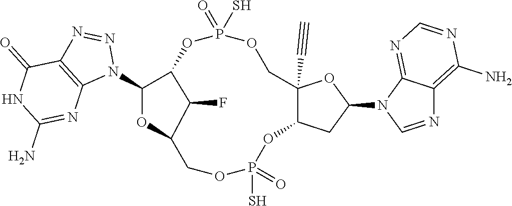 [SMILES: C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21] |
| T4-245 | 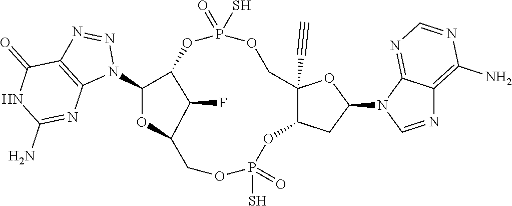 [SMILES: C#C[C@@]12COP(=O)(S)O[C@@H]3[C@@H](F)[C@@H](COP(=O)(S)O[C@H]1C[C@H](n1cnc4c(N)ncnc41)O2)O[C@H]3n1nnc2c(=O)[nH]c(N)nc21] |
| T4-246 | 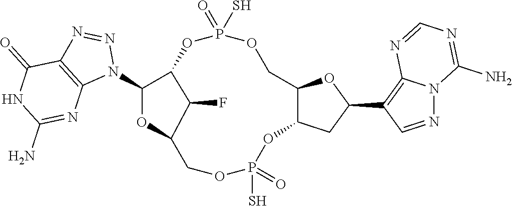 [SMILES: Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1] |
| T4-247 | 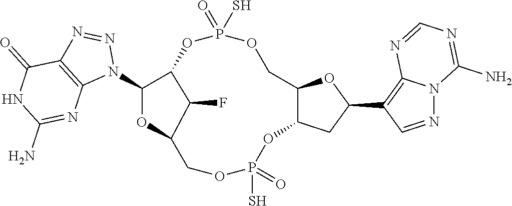 [SMILES: Nc1nc2c(nnn2[C@@H]2O[C@@H]3COP(=O)(S)O[C@H]4C[C@H](c5cnn6c(N)ncnc56)O[C@@H]4COP(=O)(S)O[C@@H]2[C@H]3F)c(=O)[nH]1] |
| T4-248 | 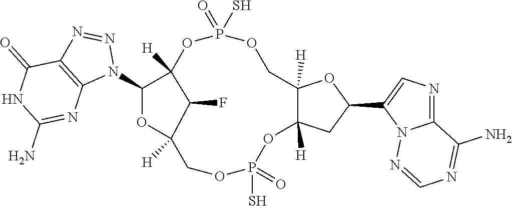 [SMILES: Nc1nc2c(nnn2[C@@H]2O[C@@H]3COP(=O)(S)O[C@H]4C[C@H](c5cnc6c(N)ncnn56)O[C@@H]4COP(=O)(S)O[C@@H]2[C@H]3F)c(=O)[nH]1] |
| T4-249 | 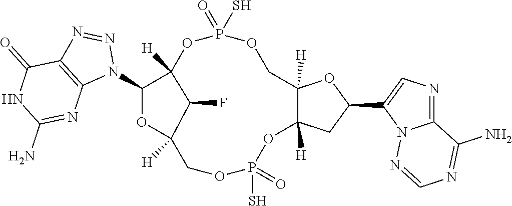 [SMILES: Nc1nc2c(nnn2[C@@H]2O[C@@H]3COP(=O)(S)O[C@H]4C[C@H](c5cnc6c(N)ncnn56)O[C@@H]4COP(=O)(S)O[C@@H]2[C@H]3F)c(=O)[nH]1] |
| T4-250 | 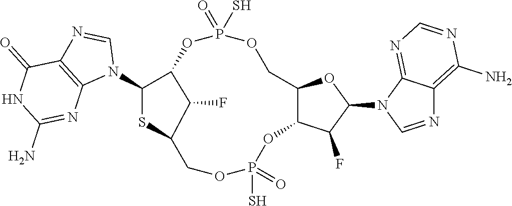 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-251 | 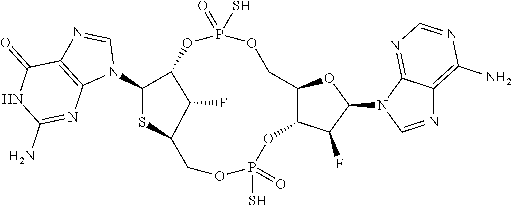 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-252 | 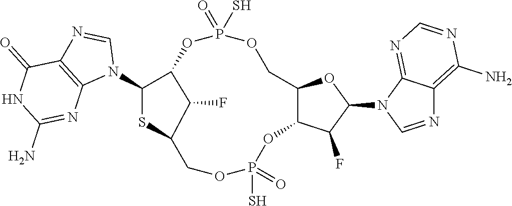 [SMILES: Nc1nc2c(ncn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-253 | 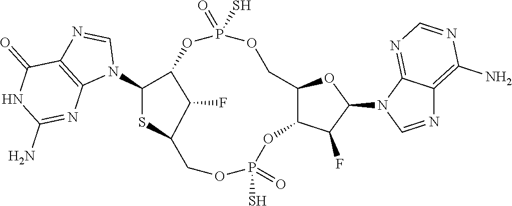 [SMILES: Nc1nc2c(ncn2[C@@H]2S[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1] |
| T4-254 | 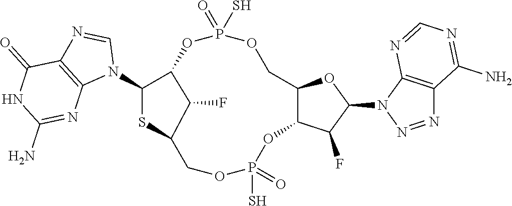 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-255 | 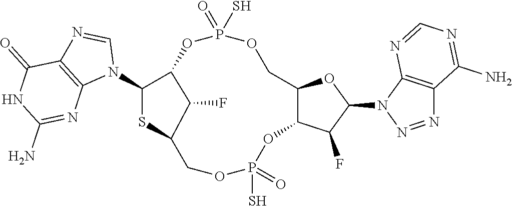 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-256 | 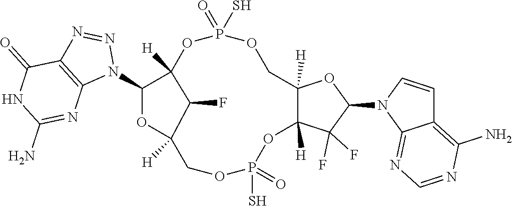 [SMILES: Nc1nc2c(nnn2[C@@H]2O[C@@H]3COP(=O)(S)O[C@@H]4[C@@H](COP(=O)(S)O[C@@H]2[C@H]3F)O[C@@H](n2ccc3c(N)ncnc32)C4(F)F)c(=O)[nH]1] |
| T4-257 | 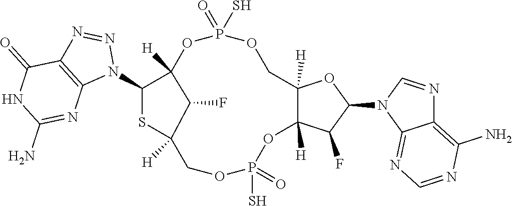 [SMILES: Nc1nc2c(nnn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-258 | 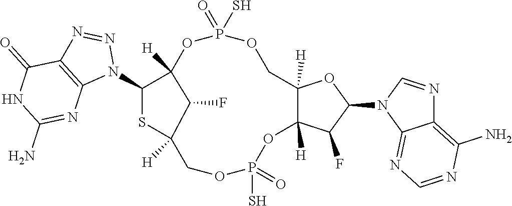 [SMILES: Nc1nc2c(nnn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-259 | 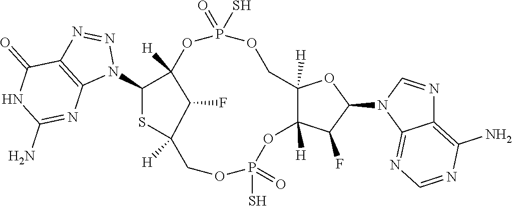 [SMILES: Nc1nc2c(nnn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-260 | 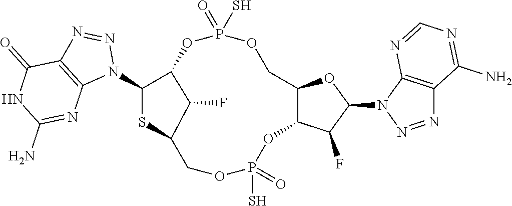 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-261 | 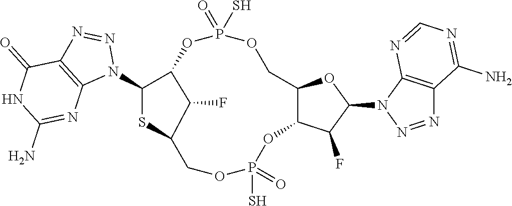 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-262 | 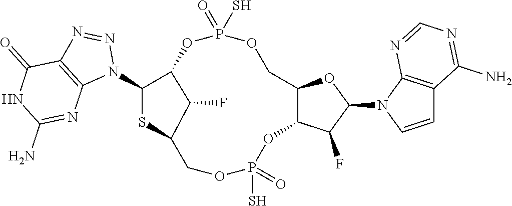 [SMILES: Nc1nc2c(nnn2[C@@H]2S[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1] |
| T4-263 | 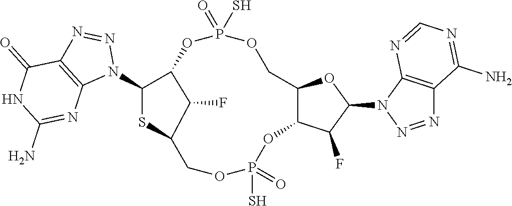 [SMILES: Nc1nc2c(nnn2[C@@H]2S[C@@H]3COP(=O)(S)O[C@H]4[C@H](F)[C@H](n5nnc6c(N)ncnc65)O[C@@H]4COP(=O)(S)O[C@@H]2[C@@H]3F)c(=O)[nH]1] |
| T4-264 | 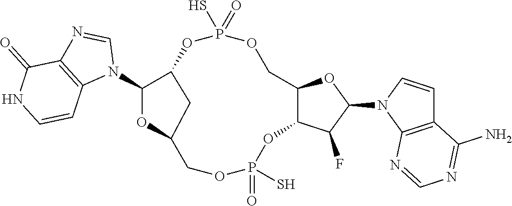 [SMILES: Nc1ncnc2c1ccn2[C@@H]1O[C@@H]2COP(=O)(S)O[C@@H]3C[C@@H](COP(=O)(S)O[C@H]2[C@@H]1F)O[C@H]3n1cnc2c(=O)[nH]ccc21] |
| T4-265 | 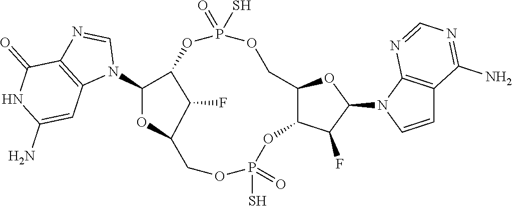 [SMILES: Nc1cc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1] |
| T4-266 | 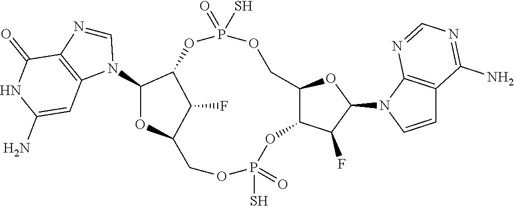 [SMILES: Nc1cc2c(ncn2[C@@H]2O[C@@H]3COP(=O)(S)O[C@H]4[C@H](F)[C@H](n5ccc6c(N)ncnc65)O[C@@H]4COP(=O)(S)O[C@@H]2[C@@H]3F)c(=O)[nH]1] |
| T4-267 | 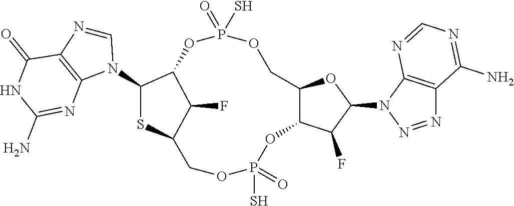 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] |
| T4-268 | 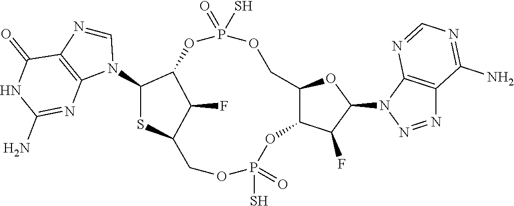 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] |
| T4-269 | 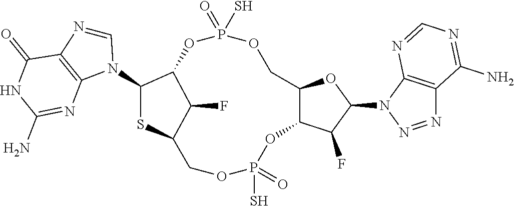 [SMILES: Nc1nc2c(ncn2[C@@H]2S[C@@H]3COP(=O)(S)O[C@H]4[C@H](F)[C@H](n5nnc6c(N)ncnc65)O[C@@H]4COP(=O)(S)O[C@@H]2[C@H]3F)c(=O)[nH]1] |
| T4-270 | 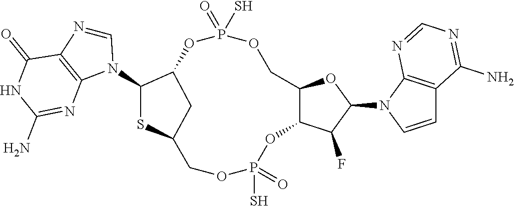 [SMILES: Nc1nc2c(ncn2[C@@H]2S[C@@H]3COP(=O)(S)O[C@H]4[C@H](F)[C@H](n5ccc6c(N)ncnc65)O[C@@H]4COP(=O)(S)O[C@@H]2C3)c(=O)[nH]1] |
| T4-271 | 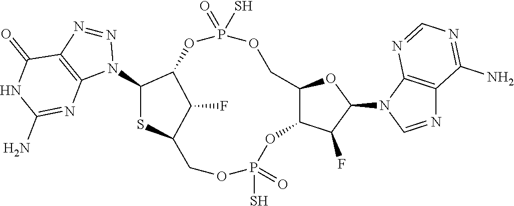 [SMILES: Nc1nc2c(nnn2[C@@H]2S[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-272 | 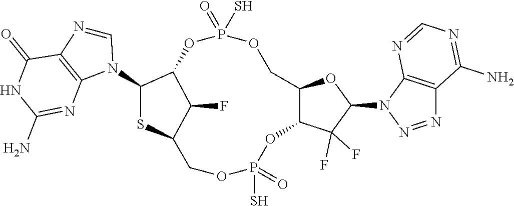 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1] |
| T4-273 | 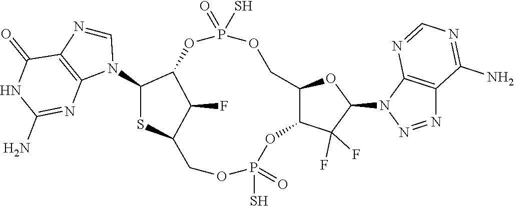 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1] |
| T4-274 | 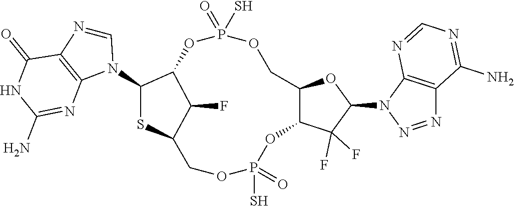 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1] |
| T4-275 | 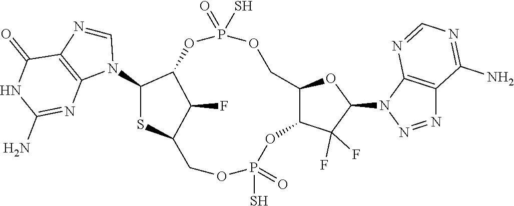 [SMILES: Nc1nc2c(ncn2[C@@H]2S[C@@H]3COP(=O)(S)O[C@@H]4[C@@H](COP(=O)(S)O[C@@H]2[C@H]3F)O[C@@H](n2nnc3c(N)ncnc32)C4(F)F)c(=O)[nH]1] |
| T4-276 | 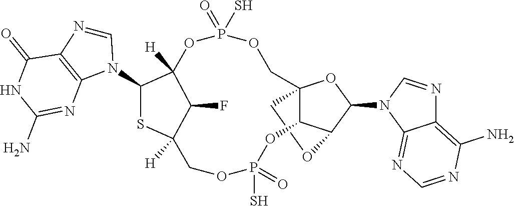 [SMILES: Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-277 | 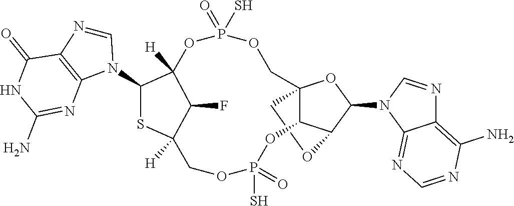 [SMILES: Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-278 | 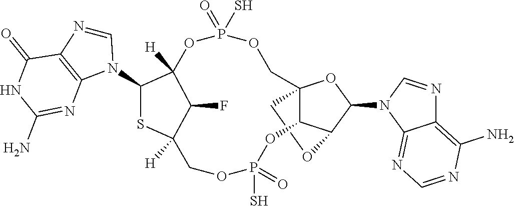 [SMILES: Nc1nc2c(ncn2[C@@H]2S[C@@H]3COP(=O)(S)O[C@H]4[C@H]5OC[C@]4(COP(=O)(S)O[C@@H]2[C@H]3F)O[C@H]5n2cnc3c(N)ncnc32)c(=O)[nH]1] |
| T4-279 | 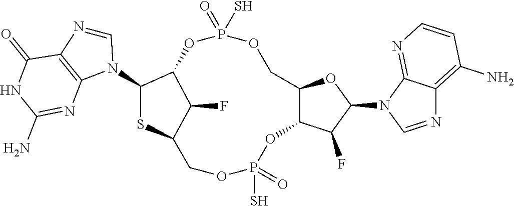 [SMILES: Nc1nc2c(ncn2[C@@H]2S[C@@H]3COP(=O)(S)O[C@H]4[C@H](F)[C@H](n5cnc6c(N)ccnc65)O[C@@H]4COP(=O)(S)O[C@@H]2[C@H]3F)c(=O)[nH]1] |
| T4-280 | 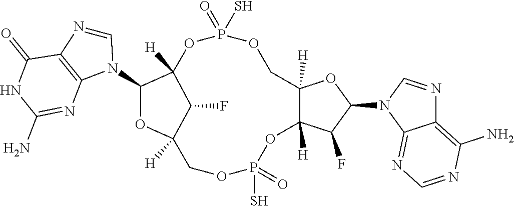 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-281 | 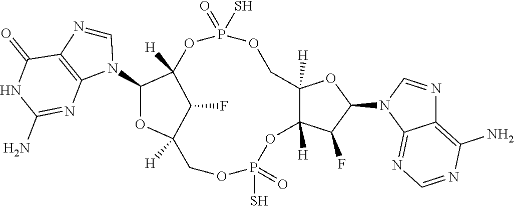 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-282 | 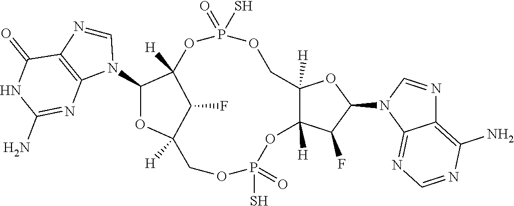 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@@H]2[C@@H]3F)c(=O)[nH]1] |
| T4-283 | 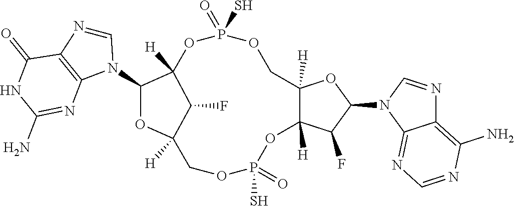 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@](=O)(S)O[C@@H]2[C@@H]3F)c(=O)[nH]1] |
| T4-284 | 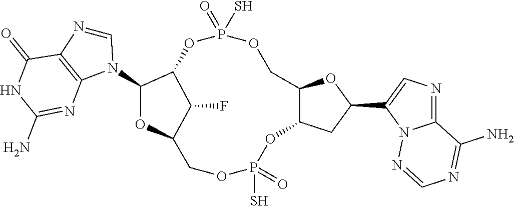 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1] |
| T4-285 | 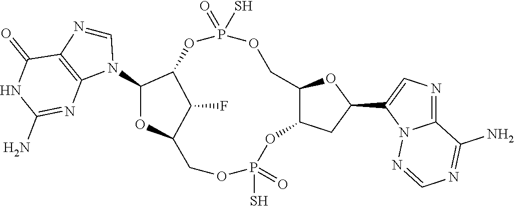 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1] |
| T4-286 | 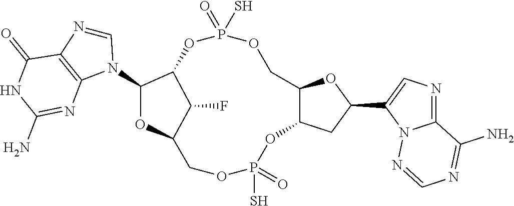 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1] |
| T4-287 | 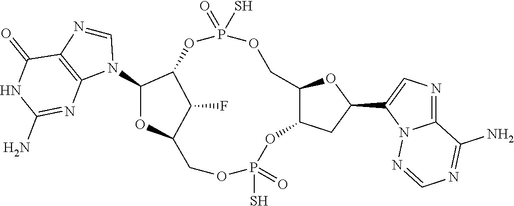 [SMILES: Nc1nc2c(ncn2[C@@H]2O[C@@H]3COP(=O)(S)O[C@H]4C[C@H](c5cnc6c(N)ncnn56)O[C@@H]4COP(=O)(S)O[C@@H]2[C@@H]3F)c(=O)[nH]1] |
| T4-288 | 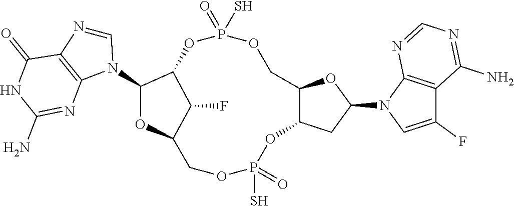 [SMILES: Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1] |
| T4-289 | 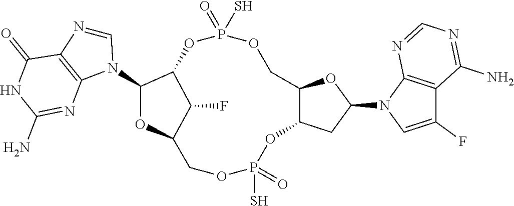 [SMILES: Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1] |
| T4-290 | 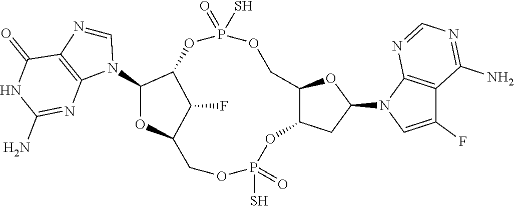 [SMILES: Nc1nc2c(ncn2[C@@H]2O[C@@H]3COP(=O)(S)O[C@H]4C[C@H](n5cc(F)c6c(N)ncnc65)O[C@@H]4COP(=O)(S)O[C@@H]2[C@@H]3F)c(=O)[nH]1] |
| T4-291 | 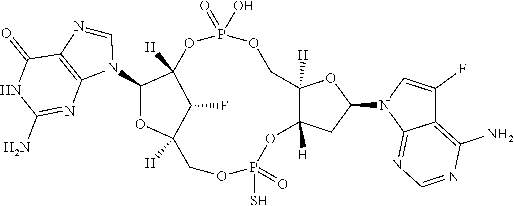 [SMILES: Nc1nc2c(ncn2[C@@H]2O[C@@H]3COP(=O)(S)O[C@H]4C[C@H](n5cc(F)c6c(N)ncnc65)O[C@@H]4COP(=O)(O)O[C@@H]2[C@@H]3F)c(=O)[nH]1] |
| T4-292 | 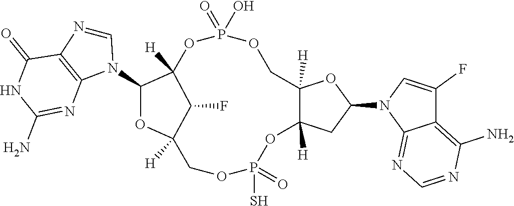 [SMILES: Nc1nc2c(ncn2[C@@H]2O[C@@H]3COP(=O)(S)O[C@H]4C[C@H](n5cc(F)c6c(N)ncnc65)O[C@@H]4COP(=O)(O)O[C@@H]2[C@@H]3F)c(=O)[nH]1] |
| T4-293 | 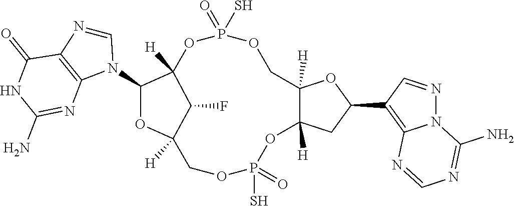 [SMILES: Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1] |
| T4-294 | 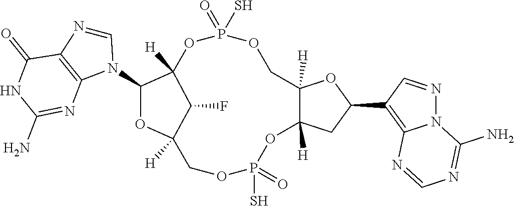 [SMILES: Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1] |
| T4-295 | 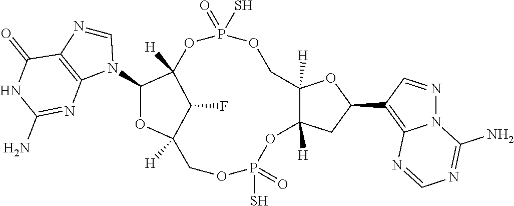 [SMILES: Nc1nc2c(ncn2[C@@H]2O[C@@H]3COP(=O)(S)O[C@H]4C[C@H](c5cnn6c(N)ncnc56)O[C@@H]4COP(=O)(S)O[C@@H]2[C@@H]3F)c(=O)[nH]1] |
| T4-296 | 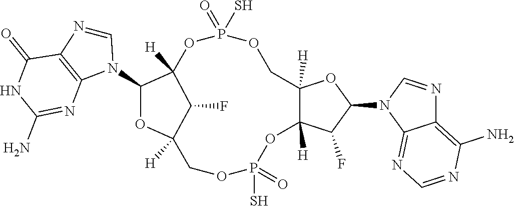 [SMILES: Nc1nc2c(ncn2[C@@H]2O[C@@H]3COP(=O)(S)O[C@H]4[C@@H](F)[C@H](n5cnc6c(N)ncnc65)O[C@@H]4COP(=O)(S)O[C@@H]2[C@@H]3F)c(=O)[nH]1] |
| T4-297 | 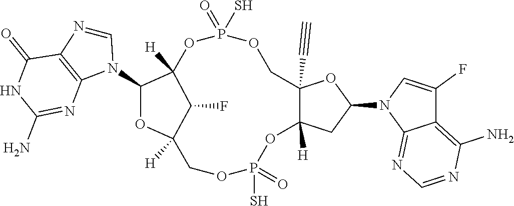 [SMILES: C#C[C@@]12COP(=O)(S)O[C@@H]3[C@H](F)[C@@H](COP(=O)(S)O[C@H]1C[C@H](n1cc(F)c4c(N)ncnc41)O2)O[C@H]3n1cnc2c(=O)[nH]c(N)nc21] |
| T4-298 | 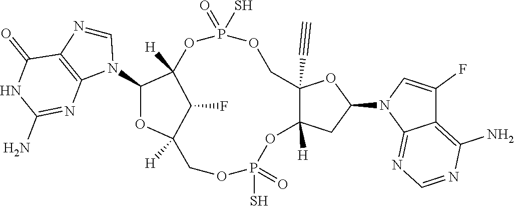 [SMILES: C#C[C@@]12COP(=O)(S)O[C@@H]3[C@H](F)[C@@H](COP(=O)(S)O[C@H]1C[C@H](n1cc(F)c4c(N)ncnc41)O2)O[C@H]3n1cnc2c(=O)[nH]c(N)nc21] |
| T4-299 | 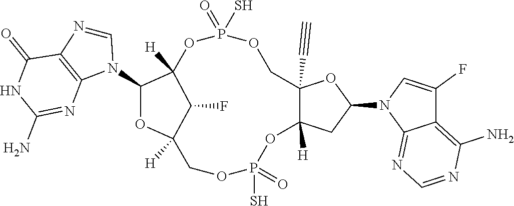 [SMILES: C#C[C@@]12COP(=O)(S)O[C@@H]3[C@H](F)[C@@H](COP(=O)(S)O[C@H]1C[C@H](n1cc(F)c4c(N)ncnc41)O2)O[C@H]3n1cnc2c(=O)[nH]c(N)nc21] |
| T4-300 | 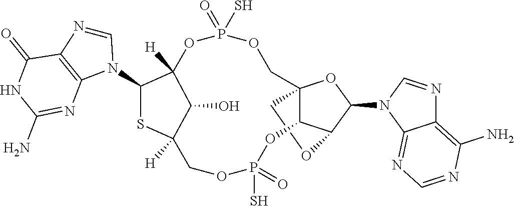 [SMILES: Nc1nc2c(ncn2[C@@H]2S[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-301 | 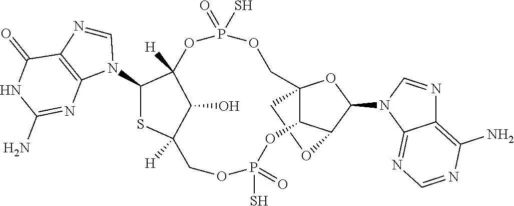 [SMILES: Nc1nc2c(ncn2[C@@H]2S[C@@H]3COP(=O)(S)O[C@H]4[C@H]5OC[C@]4(COP(=O)(S)O[C@@H]2[C@@H]3O)O[C@H]5n2cnc3c(N)ncnc32)c(=O)[nH]1] |
| T4-302 | 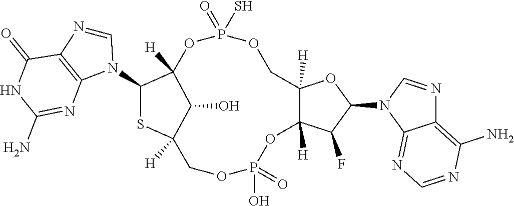 [SMILES: Nc1nc2c(ncn2[C@@H]2S[C@@H]3COP(=O)(O)O[C@H]4[C@H](F)[C@H](n5cnc6c(N)ncnc65)O[C@@H]4COP(=O)(S)O[C@@H]2[C@@H]3O)c(=O)[nH]1] |
| T4-303 | 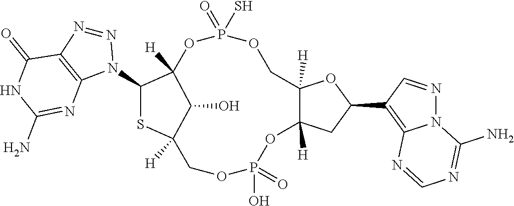 [SMILES: Nc1nc2c(nnn2[C@@H]2S[C@@H]3COP(=O)(O)O[C@H]4C[C@H](c5cnn6c(N)ncnc56)O[C@@H]4COP(=O)(S)O[C@@H]2[C@@H]3O)c(=O)[nH]1] |
| T4-304 | 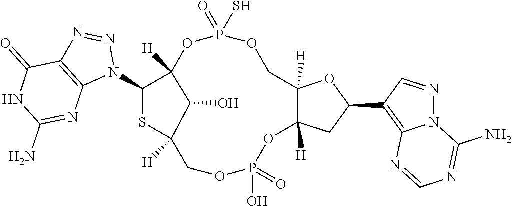 [SMILES: Nc1nc2c(nnn2[C@@H]2S[C@@H]3COP(=O)(O)O[C@H]4C[C@H](c5cnn6c(N)ncnc56)O[C@@H]4COP(=O)(S)O[C@@H]2[C@@H]3O)c(=O)[nH]1] |
| T4-305 | 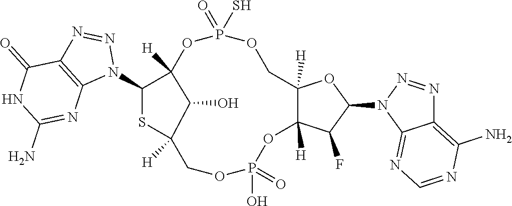 [SMILES: Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1] |
| T4-306 | 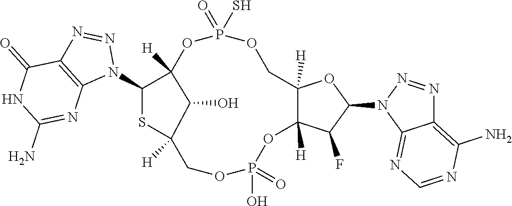 [SMILES: Nc1nc2c(nnn2[C@@H]2S[C@@H]3COP(=O)(O)O[C@H]4[C@H](F)[C@H](n5nnc6c(N)ncnc65)O[C@@H]4COP(=O)(S)O[C@@H]2[C@@H]3O)c(=O)[nH]1] |
| T4-307 | 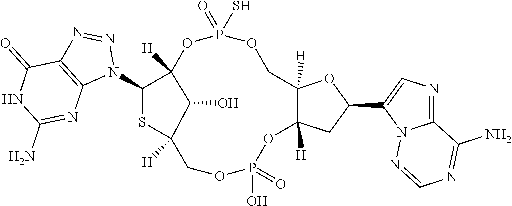 [SMILES: Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1] |
| T4-308 | 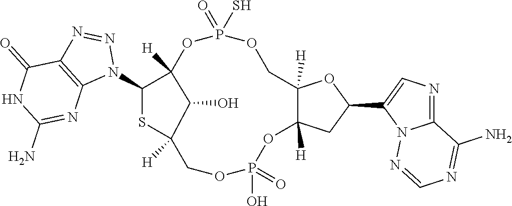 [SMILES: Nc1nc2c(nnn2[C@@H]2S[C@@H]3COP(=O)(O)O[C@H]4C[C@H](c5cnc6c(N)ncnn56)O[C@@H]4COP(=O)(S)O[C@@H]2[C@@H]3O)c(=O)[nH]1] |
| T4-309 | 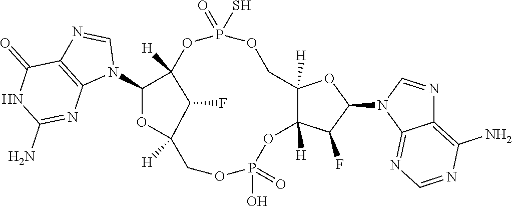 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@@H]2[C@@H]3F)c(=O)[nH]1] |
| T4-310 | 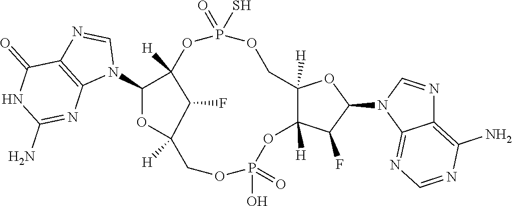 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@@H]2[C@@H]3F)c(=O)[nH]1] |
| T4-311 | 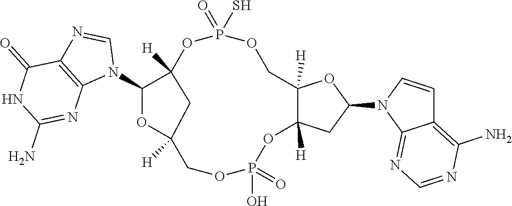 [SMILES: Nc1nc2c(ncn2[C@@H]2O[C@@H]3COP(=O)(O)O[C@H]4C[C@H](n5ccc6c(N)ncnc65)O[C@@H]4COP(=O)(S)O[C@@H]2C3)c(=O)[nH]1] |
| T4-312 | 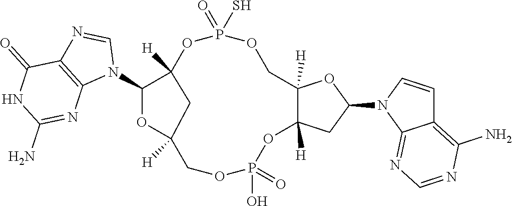 [SMILES: Nc1nc2c(ncn2[C@@H]2O[C@@H]3COP(=O)(O)O[C@H]4C[C@H](n5ccc6c(N)ncnc65)O[C@@H]4COP(=O)(S)O[C@@H]2C3)c(=O)[nH]1] |
| T4-313 | 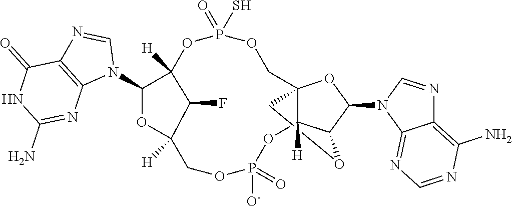 [SMILES: Nc1nc2c(ncn2[C@@H]2O[C@@H]3COP(=O)([O-])O[C@H]4[C@H]5OC[C@]4(COP(=O)(S)O[C@@H]2[C@H]3F)O[C@H]5n2cnc3c(N)ncnc32)c(=O)[nH]1] |
| T4-314 | 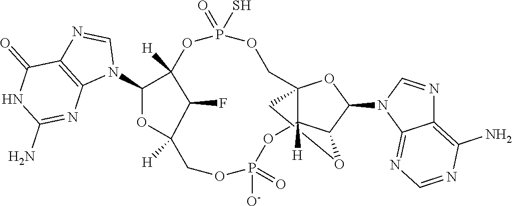 [SMILES: Nc1nc2c(ncn2[C@@H]2O[C@@H]3COP(=O)([O-])O[C@H]4[C@H]5OC[C@]4(COP(=O)(S)O[C@@H]2[C@H]3F)O[C@H]5n2cnc3c(N)ncnc32)c(=O)[nH]1] |
| T4-315 | 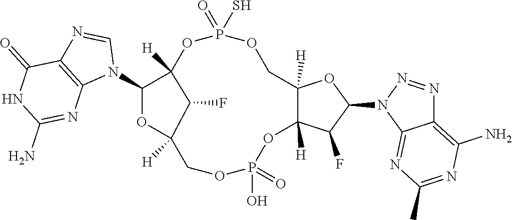 |
| T4-316 | 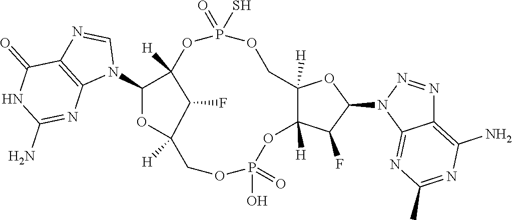 |
| T4-317 | 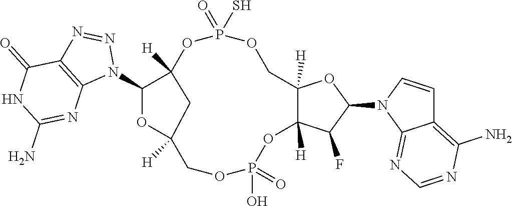 [SMILES: Nc1nc2c(nnn2[C@@H]2O[C@@H]3COP(=O)(O)O[C@H]4[C@H](F)[C@H](n5ccc6c(N)ncnc65)O[C@@H]4COP(=O)(S)O[C@@H]2C3)c(=O)[nH]1] |
| T4-318 | 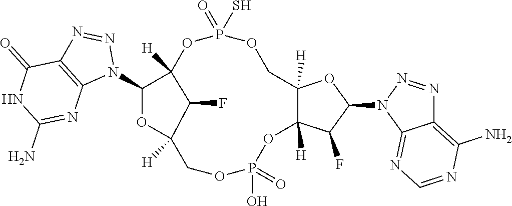 [SMILES: Nc1nc2c(nnn2[C@@H]2O[C@@H]3COP(=O)(O)O[C@H]4[C@H](F)[C@H](n5nnc6c(N)ncnc65)O[C@@H]4COP(=O)(S)O[C@@H]2[C@H]3F)c(=O)[nH]1] |
| T4-319 | 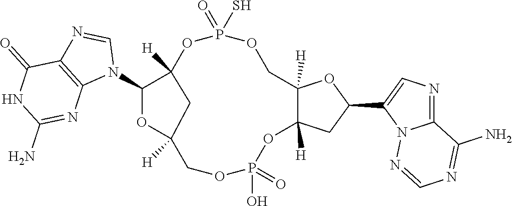 [SMILES: Nc1nc2c(ncn2[C@@H]2O[C@@H]3COP(=O)(O)O[C@H]4C[C@H](c5cnc6c(N)ncnn56)O[C@@H]4COP(=O)(S)O[C@@H]2C3)c(=O)[nH]1] |
| T4-320 | 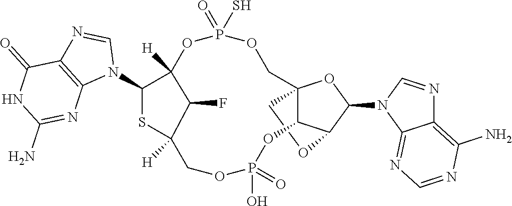 [SMILES: Nc1nc2c(ncn2[C@@H]2S[C@@H]3COP(=O)(O)O[C@H]4[C@H]5OC[C@]4(COP(=O)(S)O[C@@H]2[C@H]3F)O[C@H]5n2cnc3c(N)ncnc32)c(=O)[nH]1] |
| T4-321 | 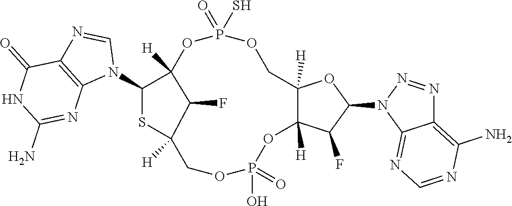 [SMILES: Nc1nc2c(ncn2[C@@H]2S[C@@H]3COP(=O)(O)O[C@H]4[C@H](F)[C@H](n5nnc6c(N)ncnc65)O[C@@H]4COP(=O)(S)O[C@@H]2[C@H]3F)c(=O)[nH]1] |
| T4-322 | 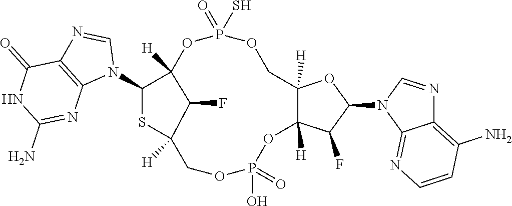 [SMILES: Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1] |
| T4-323 | 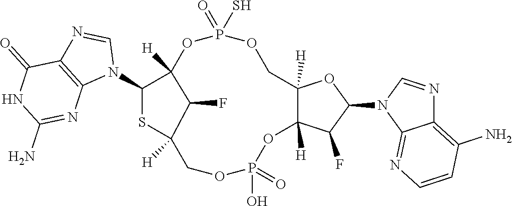 [SMILES: Nc1nc2c(ncn2[C@@H]2S[C@@H]3COP(=O)(O)O[C@H]4[C@H](F)[C@H](n5cnc6c(N)ccnc65)O[C@@H]4COP(=O)(S)O[C@@H]2[C@H]3F)c(=O)[nH]1] |
| T4-324 | 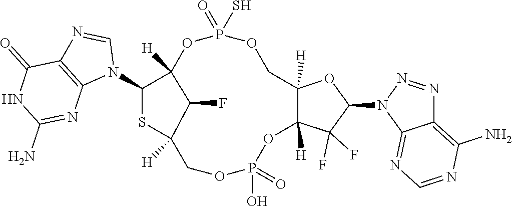 [SMILES: Nc1nc2c(ncn2[C@@H]2S[C@@H]3COP(=O)(O)O[C@@H]4[C@@H](COP(=O)(S)O[C@@H]2[C@H]3F)O[C@@H](n2nnc3c(N)ncnc32)C4(F)F)c(=O)[nH]1] |
| T4-325 | 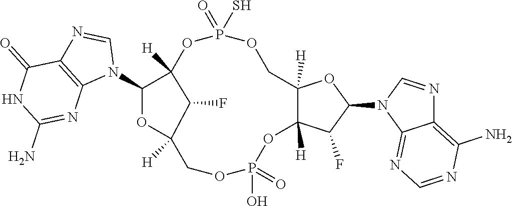 [SMILES: Nc1nc2c(ncn2[C@@H]2O[C@@H]3COP(=O)(O)O[C@H]4[C@@H](F)[C@H](n5cnc6c(N)ncnc65)O[C@@H]4COP(=O)(S)O[C@@H]2[C@@H]3F)c(=O)[nH]1] |
| T4-326 | 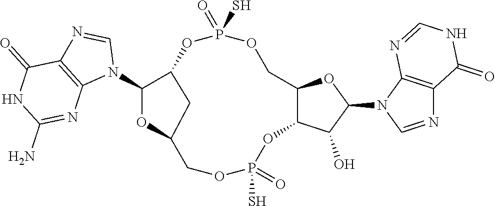 [SMILES: Nc1nc2c(ncn2[C@@H]2O[C@@H]3CO[P@@](=O)(S)O[C@H]4[C@@H](O)[C@H](n5cnc6c(=O)[nH]cnc65)O[C@@H]4CO[P@](=O)(S)O[C@@H]2C3)c(=O)[nH]1] |
| T4-327 | 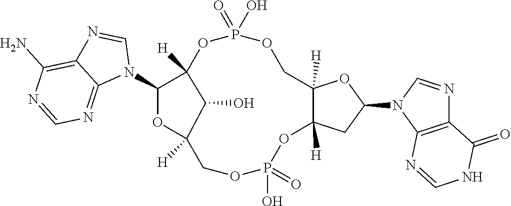 [SMILES: Nc1ncnc2c1ncn2[C@@H]1O[C@@H]2COP(=O)(O)O[C@H]3C[C@H](n4cnc5c(=O)[nH]cnc54)O[C@@H]3COP(=O)(O)O[C@@H]1[C@@H]2O] |
| T4-328 | 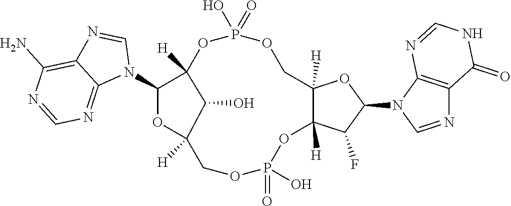 [SMILES: Nc1ncnc2c1ncn2[C@@H]1O[C@@H]2COP(=O)(O)O[C@H]3[C@@H](F)[C@H](n4cnc5c(=O)[nH]cnc54)O[C@@H]3COP(=O)(O)O[C@@H]1[C@@H]2O] |
| T4-329 | 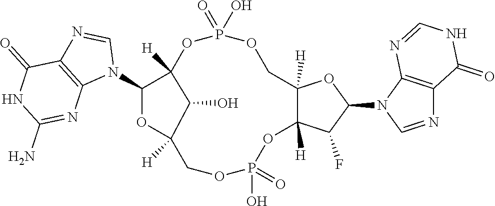 [SMILES: Nc1nc2c(ncn2[C@@H]2O[C@@H]3COP(=O)(O)O[C@H]4[C@@H](F)[C@H](n5cnc6c(=O)[nH]cnc65)O[C@@H]4COP(=O)(O)O[C@@H]2[C@@H]3O)c(=O)[nH]1] |
| T4-330 | 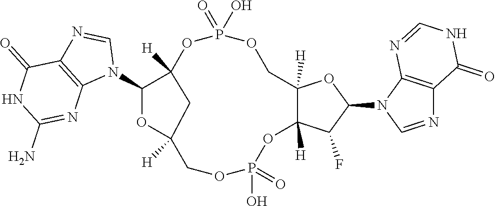 [SMILES: Nc1nc2c(ncn2[C@@H]2O[C@@H]3COP(=O)(O)O[C@H]4[C@@H](F)[C@H](n5cnc6c(=O)[nH]cnc65)O[C@@H]4COP(=O)(O)O[C@@H]2C3)c(=O)[nH]1] |
| T4-331 | 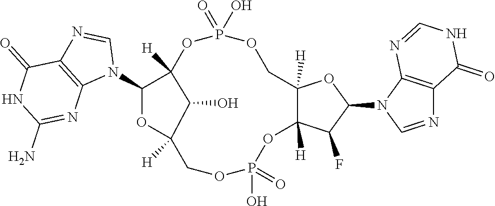 [SMILES: Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2[C@@H]3O)c(=O)[nH]1] |
| T4-332 | 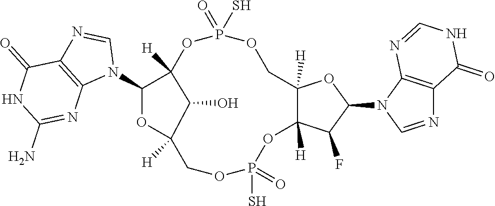 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3O)c(=O)[nH]1] |
| T4-333 | 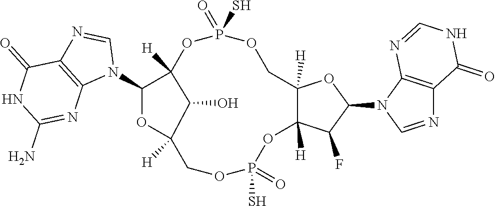 [SMILES: Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@](=O)(S)O[C@@H]2[C@@H]3O)c(=O)[nH]1] |
| T4-334 | 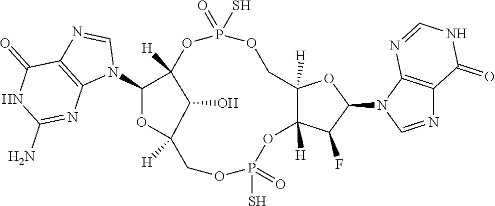 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3O)c(=O)[nH]1] |
| T4-335 | 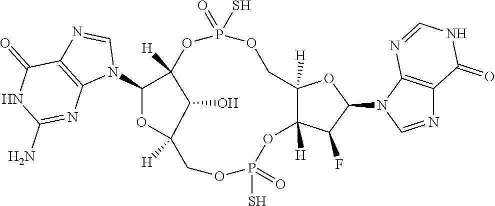 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3O)c(=O)[nH]1] |
| T4-336 | 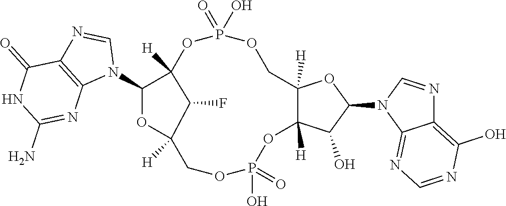 [SMILES: Nc1nc2c(ncn2[C@@H]2O[C@@H]3COP(=O)(O)O[C@H]4[C@@H](O)[C@H](n5cnc6c(O)ncnc65)O[C@@H]4COP(=O)(O)O[C@@H]2[C@@H]3F)c(=O)[nH]1] |
| T4-337 | 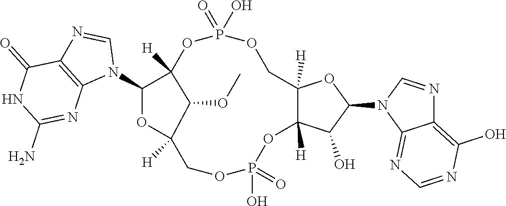 [SMILES: CO[C@H]1[C@H]2OP(=O)(O)OC[C@H]3O[C@@H](n4cnc5c(O)ncnc54)[C@H](O)[C@@H]3OP(=O)(O)OC[C@H]1O[C@H]2n1cnc2c(=O)[nH]c(N)nc21] |
| T4-338 | 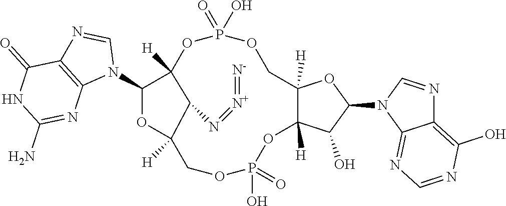 [SMILES: [N-]=[N+]=N[C@H]1[C@H]2OP(=O)(O)OC[C@H]3O[C@@H](n4cnc5c(O)ncnc54)[C@H](O)[C@@H]3OP(=O)(O)OC[C@H]1O[C@H]2n1cnc2c(=O)[nH]c(N)nc21] |
| T4-339 | 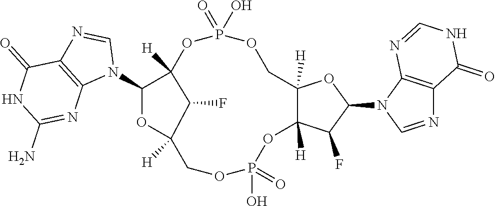 [SMILES: Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2[C@@H]3F)c(=O)[nH]1] |
| T4-340 | 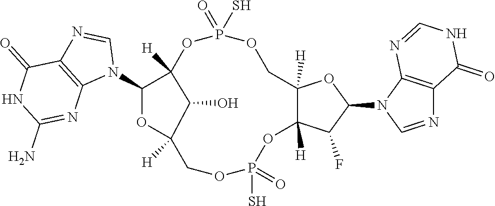 [SMILES: Nc1nc2c(ncn2[C@@H]2O[C@@H]3COP(=O)(S)O[C@H]4[C@@H](F)[C@H](n5cnc6c(=O)[nH]cnc65)O[C@@H]4COP(=O)(S)O[C@@H]2[C@@H]3O)c(=O)[nH]1] |
| T4-341 | 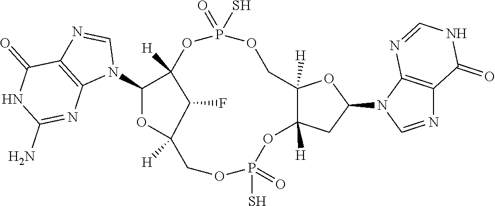 [SMILES: Nc1nc2c(ncn2[C@@H]2O[C@@H]3COP(=O)(S)O[C@H]4C[C@H](n5cnc6c(=O)[nH]cnc65)O[C@@H]4COP(=O)(S)O[C@@H]2[C@@H]3F)c(=O)[nH]1] |
| T4-342 | 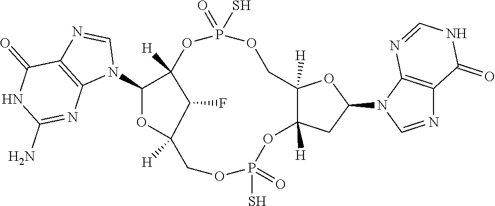 [SMILES: Nc1nc2c(ncn2[C@@H]2O[C@@H]3COP(=O)(S)O[C@H]4C[C@H](n5cnc6c(=O)[nH]cnc65)O[C@@H]4COP(=O)(S)O[C@@H]2[C@@H]3F)c(=O)[nH]1] |
| T4-343 | 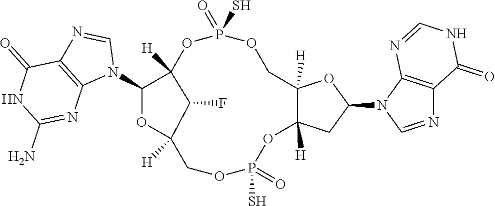 [SMILES: Nc1nc2c(ncn2[C@@H]2O[C@@H]3CO[P@@](=O)(S)O[C@H]4C[C@H](n5cnc6c(=O)[nH]cnc65)O[C@@H]4CO[P@](=O)(S)O[C@@H]2[C@@H]3F)c(=O)[nH]1] |
| T4-344 | 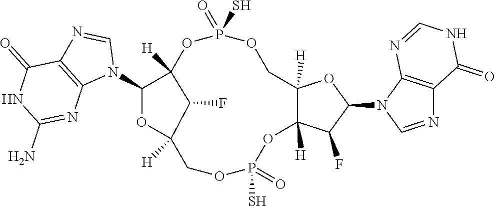 [SMILES: Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@](=O)(S)O[C@@H]2[C@@H]3F)c(=O)[nH]1] |
| T4-345 | 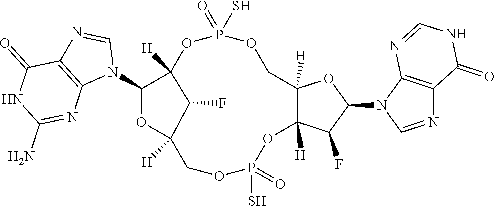 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1] |
| T4-346 | 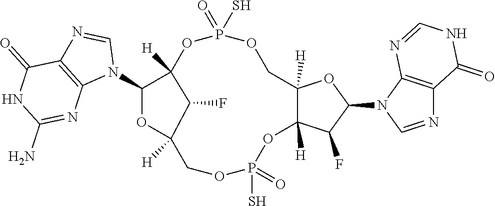 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1] |
| T4-347 | 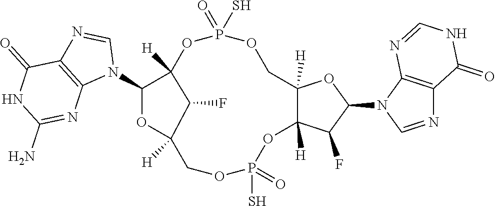 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1] |
| T4-348 | 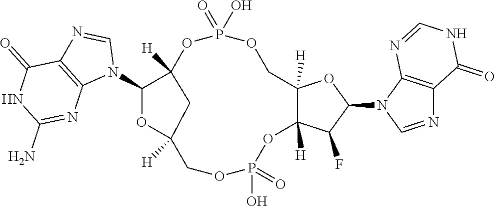 [SMILES: Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)O[C@@H]2C3)c(=O)[nH]1] |
| T4-349 | 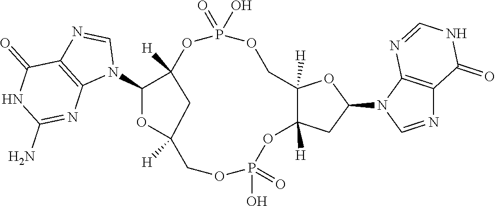 [SMILES: Nc1nc2c(ncn2[C@@H]2O[C@@H]3COP(=O)(O)O[C@H]4C[C@H](n5cnc6c(=O)[nH]cnc65)O[C@@H]4COP(=O)(O)O[C@@H]2C3)c(=O)[nH]1] |
| T4-350 | 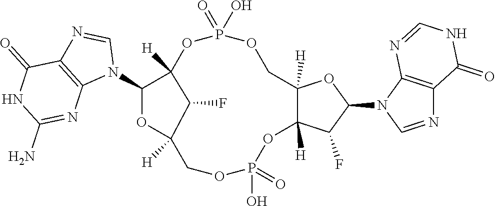 [SMILES: Nc1nc2c(ncn2[C@@H]2O[C@@H]3COP(=O)(O)O[C@H]4[C@@H](F)[C@H](n5cnc6c(=O)[nH]cnc65)O[C@@H]4COP(=O)(O)O[C@@H]2[C@@H]3F)c(=O)[nH]1] |
| T4-351 | 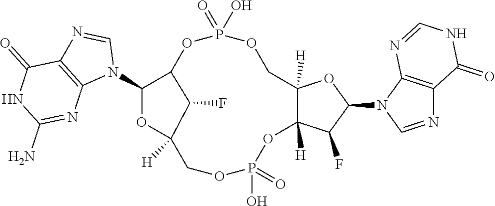 [SMILES: Nc1nc2c(ncn2[C@@H]2O[C@@H]3COP(=O)(O)O[C@H]4[C@H](F)[C@H](n5cnc6c(=O)[nH]cnc65)O[C@@H]4COP(=O)(O)OC2[C@@H]3F)c(=O)[nH]1] |
| T4-352 | 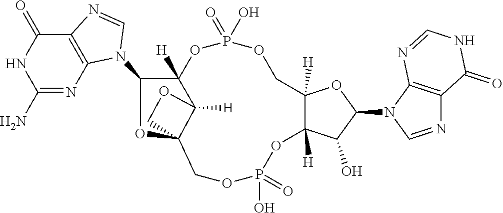 [SMILES: Nc1nc2c(ncn2[C@@H]2O[C@@]34CO[C@H]3[C@H]2OP(=O)(O)OC[C@H]2O[C@@H](n3cnc5c(=O)[nH]cnc53)[C@H](O)[C@@H]2OP(=O)(O)OC4)c(=O)[nH]1] |
| T4-353 | 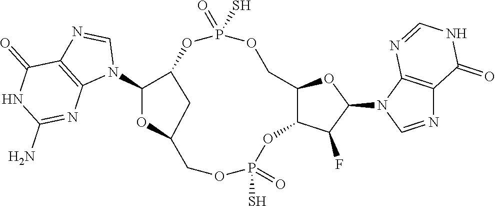 [SMILES: Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@@](=O)(S)O[C@@H]2C3)c(=O)[nH]1] |
| T4-354 | 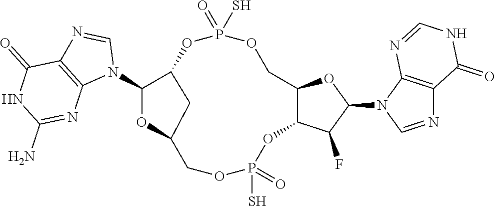 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2C3)c(=O)[nH]1] |
| T4-355 | 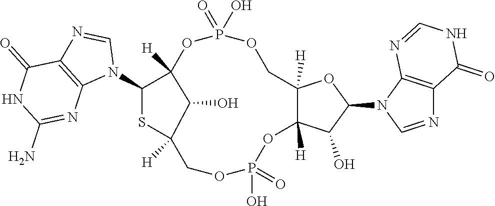 [SMILES: Nc1nc2c(ncn2[C@@H]2S[C@@H]3COP(=O)(O)O[C@H]4[C@@H](O)[C@H](n5cnc6c(=O)[nH]cnc65)O[C@@H]4COP(=O)(O)O[C@@H]2[C@@H]3O)c(=O)[nH]1] |
| T4-356 | 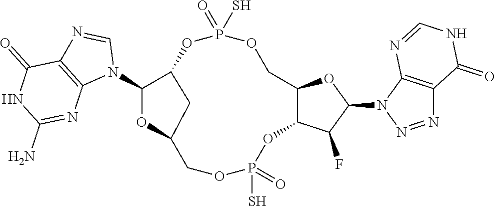 [SMILES: Nc1nc2c(ncn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1] |
| T4-357 | 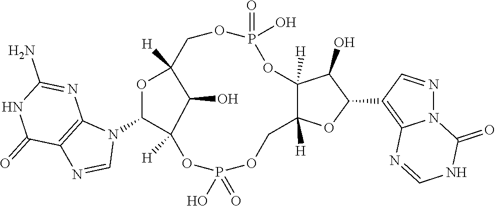 [SMILES: Nc1nc2c(ncn2[C@@H]2O[C@@H]3COP(=O)(O)O[C@H]4[C@@H](O)[C@H](c5cnn6c(=O)[nH]cnc56)O[C@@H]4COP(=O)(O)O[C@@H]2[C@@H]3O)c(=O)[nH]1] |
| T4-358 | 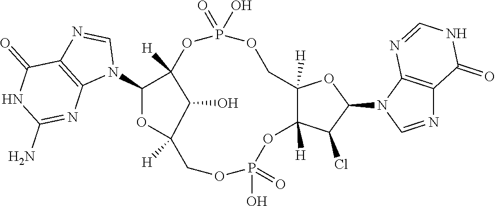 [SMILES: Nc1nc2c(ncn2[C@@H]2O[C@@H]3COP(=O)(O)O[C@H]4[C@H](Cl)[C@H](n5cnc6c(=O)[nH]cnc65)O[C@@H]4COP(=O)(O)O[C@@H]2[C@@H]3O)c(=O)[nH]1] |
| T4-359 | 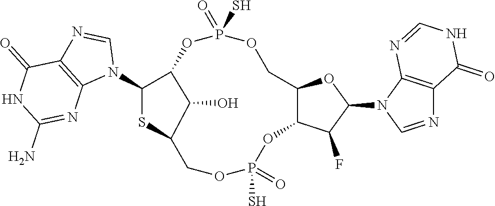 [SMILES: Nc1nc2c(ncn2[C@@H]2S[C@@H]3CO[P@@](=O)(S)O[C@H]4[C@H](F)[C@H](n5cnc6c(=O)[nH]cnc65)O[C@@H]4CO[P@](=O)(S)O[C@@H]2[C@@H]3O)c(=O)[nH]1] |
| T4-360 | 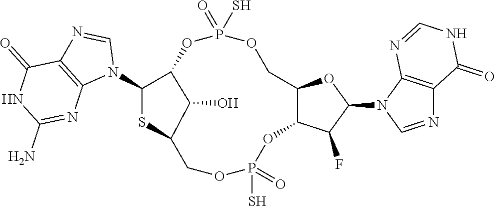 [SMILES: Nc1nc2c(ncn2[C@@H]2S[C@@H]3COP(=O)(S)O[C@H]4[C@H](F)[C@H](n5cnc6c(=O)[nH]cnc65)O[C@@H]4COP(=O)(S)O[C@@H]2[C@@H]3O)c(=O)[nH]1] |
| T4-361 | 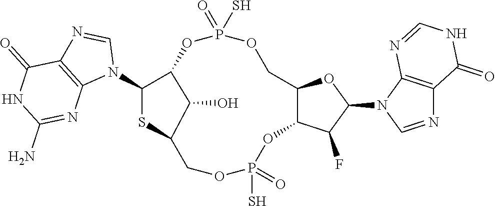 [SMILES: Nc1nc2c(ncn2[C@@H]2S[C@@H]3COP(=O)(S)O[C@H]4[C@H](F)[C@H](n5cnc6c(=O)[nH]cnc65)O[C@@H]4COP(=O)(S)O[C@@H]2[C@@H]3O)c(=O)[nH]1] |
| T4-362 | 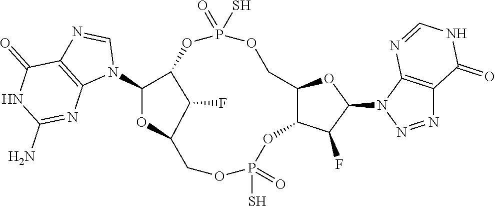 [SMILES: Nc1nc2c(ncn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@@H]3F)c(=O)[nH]1] |
| T4-363 | 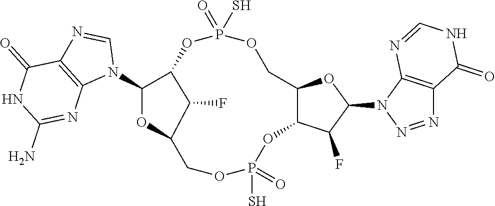 [SMILES: Nc1nc2c(ncn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@@H]3F)c(=O)[nH]1] |
| T4-364 | 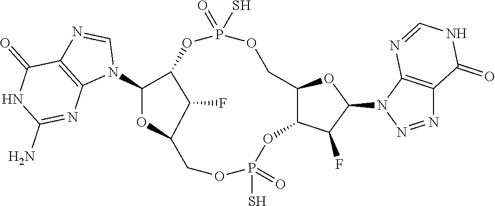 [SMILES: Nc1nc2c(ncn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@@H]3F)c(=O)[nH]1] |
| T4-365 | 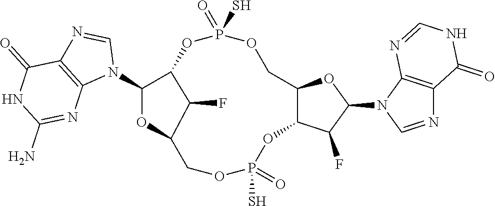 [SMILES: Nc1nc2c(ncn2[C@@H]2O[C@@H]3CO[P@@](=O)(S)O[C@H]4[C@H](F)[C@H](n5cnc6c(=O)[nH]cnc65)O[C@@H]4CO[P@](=O)(S)O[C@@H]2[C@H]3F)c(=O)[nH]1] |
| T4-366 | 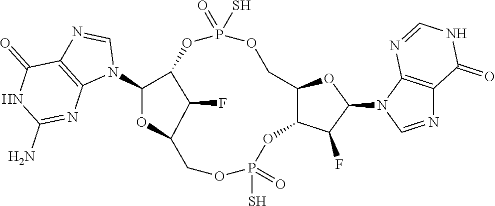 [SMILES: Nc1nc2c(ncn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@H]3F)c(=O)[nH]1] |
| T4-367 | 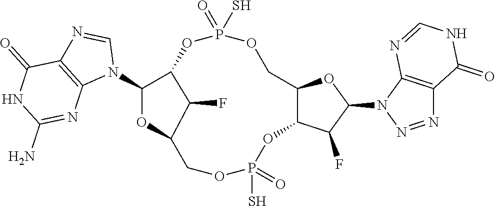 [SMILES: Nc1nc2c(ncn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@H]3F)c(=O)[nH]1] |
| T4-368 | 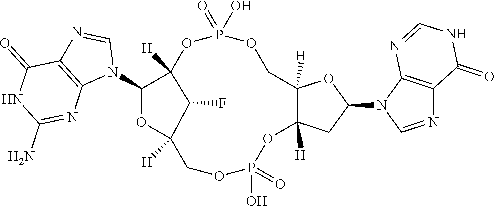 [SMILES: Nc1nc2c(ncn2[C@@H]2O[C@@H]3COP(=O)(O)O[C@H]4C[C@H](n5cnc6c(=O)[nH]cnc65)O[C@@H]4COP(=O)(O)O[C@@H]2[C@@H]3F)c(=O)[nH]1] |
| T4-369 | 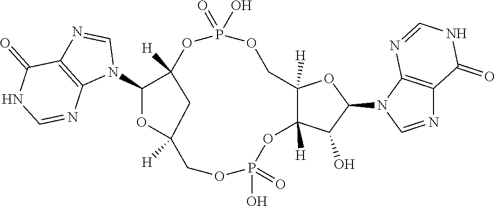 [SMILES: O=c1[nH]cnc2c1ncn2[C@@H]1O[C@@H]2COP(=O)(O)O[C@@H]3C[C@@H](COP(=O)(O)O[C@H]2[C@H]1O)O[C@H]3n1cnc2c(=O)[nH]cnc21] |
| T4-370 | 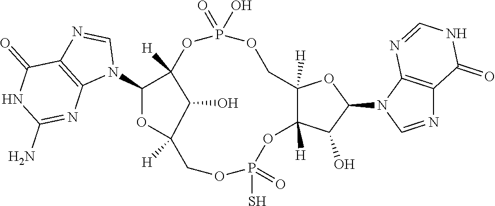 [SMILES: Nc1nc2c(ncn2[C@@H]2O[C@@H]3COP(=O)(S)O[C@H]4[C@@H](O)[C@H](n5cnc6c(=O)[nH]cnc65)O[C@@H]4COP(=O)(O)O[C@@H]2[C@@H]3O)c(=O)[nH]1] |
| T4-371 | 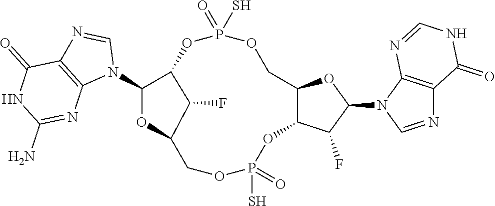 [SMILES: Nc1nc2c(ncn2[C@@H]2O[C@@H]3COP(=O)(S)O[C@H]4[C@@H](F)[C@H](n5cnc6c(=O)[nH]cnc65)O[C@@H]4COP(=O)(S)O[C@@H]2[C@@H]3F)c(=O)[nH]1] |
| T4-372 | 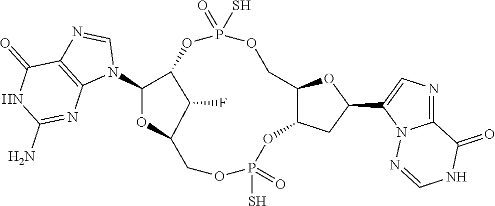 [SMILES: Nc1nc2c(ncn2[C@@H]2O[C@@H]3COP(=O)(S)O[C@H]4C[C@H](c5cnc6c(=O)[nH]cnn56)O[C@@H]4COP(=O)(S)O[C@@H]2[C@@H]3F)c(=O)[nH]1] |
| T4-373 | 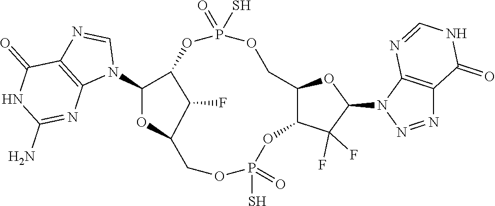 [SMILES: Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1] |
| T4-374 | 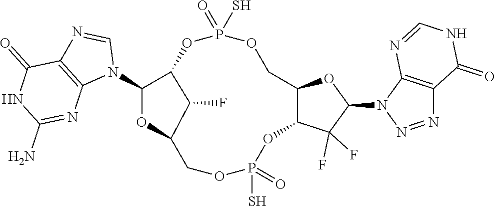 [SMILES: Nc1nc2c(ncn2[C@@H]2O[C@@H]3COP(=O)(S)O[C@@H]4[C@@H](COP(=O)(S)O[C@@H]2[C@@H]3F)O[C@@H](n2nnc3c(=O)[nH]cnc32)C4(F)F)c(=O)[nH]1] |
| T4-375 | 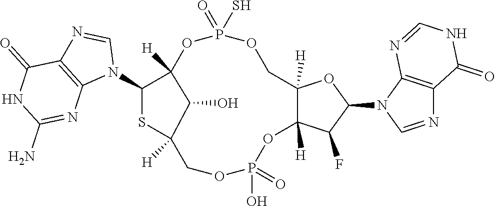 [SMILES: Nc1nc2c(ncn2[C@@H]2S[C@@H]3COP(=O)(O)O[C@H]4[C@H](F)[C@H](n5cnc6c(=O)[nH]cnc65)O[C@@H]4COP(=O)(S)O[C@@H]2[C@@H]3O)c(=O)[nH]1] |
| T4-376 | 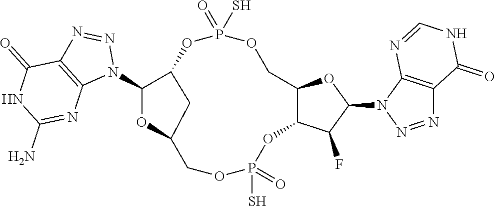 [SMILES: Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1] |
| T4-377 | 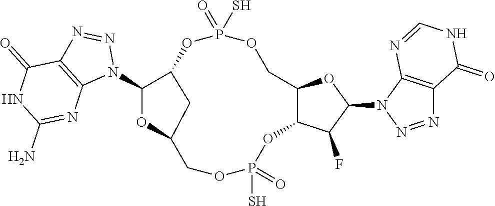 [SMILES: Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2C3)c(=O)[nH]1] |
| T4-378 | 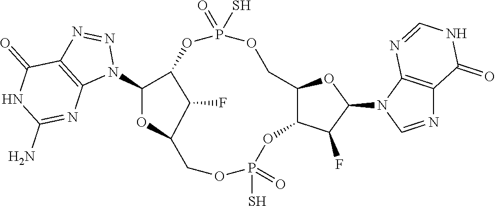 [SMILES: Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1] |
| T4-379 | 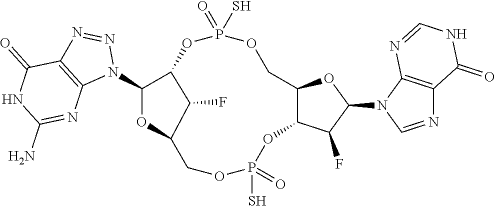 [SMILES: Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@@H]3F)c(=O)[nH]1] |
| T4-380 | 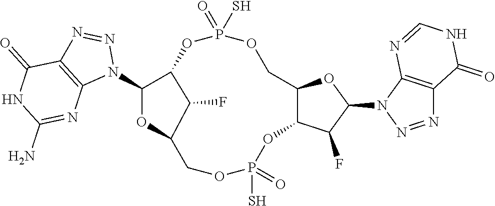 [SMILES: Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@@H]3F)c(=O)[nH]1] |
| T4-381 | 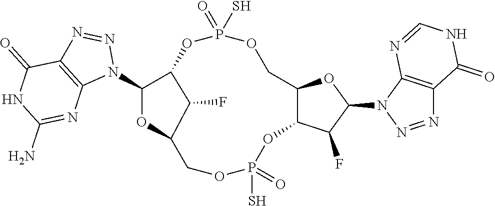 [SMILES: Nc1nc2c(nnn2[C@@H]2O[C@@H]3COP(=O)(S)O[C@H]4[C@H](F)[C@H](n5nnc6c(=O)[nH]cnc65)O[C@@H]4COP(=O)(S)O[C@@H]2[C@@H]3F)c(=O)[nH]1] |
| T4-382 | 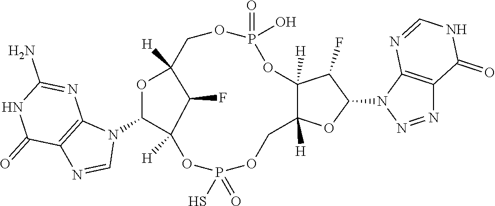 [SMILES: Nc1nc2c(ncn2[C@@H]2O[C@@H]3COP(=O)(O)O[C@H]4[C@H](F)[C@H](n5nnc6c(=O)[nH]cnc65)O[C@@H]4COP(=O)(S)O[C@@H]2[C@@H]3F)c(=O)[nH]1] |
| T4-383 | 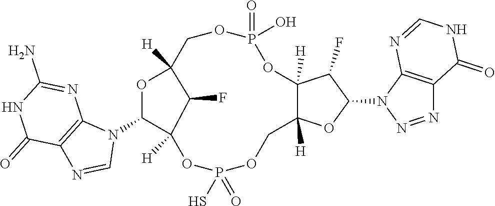 [SMILES: Nc1nc2c(ncn2[C@@H]2O[C@@H]3COP(=O)(O)O[C@H]4[C@H](F)[C@H](n5nnc6c(=O)[nH]cnc65)O[C@@H]4COP(=O)(S)O[C@@H]2[C@@H]3F)c(=O)[nH]1] |
| T4-384 | 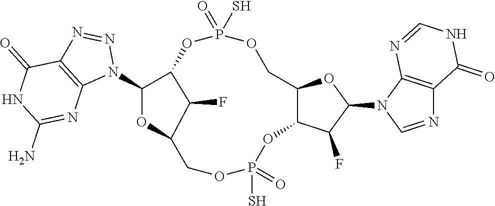 [SMILES: Nc1nc2c(nnn2[C@@H]2O[C@@H]3COP(=O)(S)O[C@H]4[C@H](F)[C@H](n5cnc6c(=O)[nH]cnc65)O[C@@H]4COP(=O)(S)O[C@@H]2[C@H]3F)c(=O)[nH]1] |
| T4-385 | 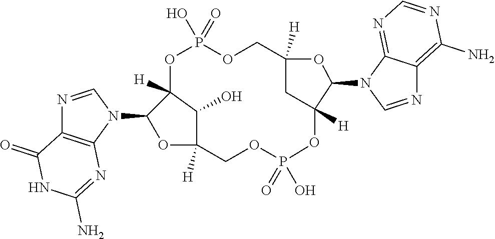 [SMILES: Nc1nc2c(ncn2[C@@H]2O[C@@H]3COP(=O)(O)O[C@@H]4C[C@@H](COP(=O)(O)O[C@@H]2[C@@H]3O)O[C@H]4n2cnc3c(N)ncnc32)c(=O)[nH]1] |
| T4-386 | 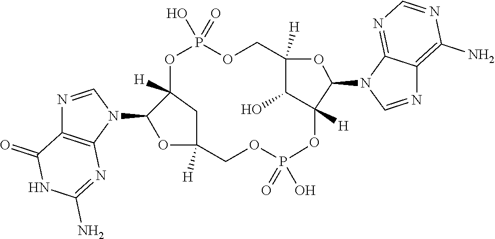 [SMILES: Nc1nc2c(ncn2[C@@H]2O[C@@H]3COP(=O)(O)O[C@@H]4[C@H](O)[C@@H](COP(=O)(O)O[C@@H]2C3)O[C@H]4n2cnc3c(N)ncnc32)c(=O)[nH]1] |
| T4-387 | 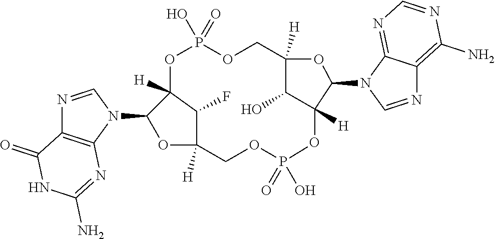 [SMILES: Nc1nc2c(ncn2[C@@H]2O[C@@H]3COP(=O)(O)O[C@@H]4[C@H](O)[C@@H](COP(=O)(O)O[C@@H]2[C@@H]3F)O[C@H]4n2cnc3c(N)ncnc32)c(=O)[nH]1] |
| T4-388 | 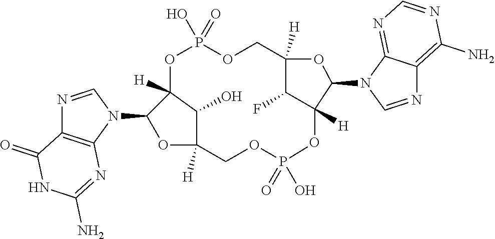 [SMILES: Nc1nc2c(ncn2[C@@H]2O[C@@H]3COP(=O)(O)O[C@@H]4[C@H](F)[C@@H](COP(=O)(O)O[C@@H]2[C@@H]3O)O[C@H]4n2cnc3c(N)ncnc32)c(=O)[nH]1] |
| T4-389 | 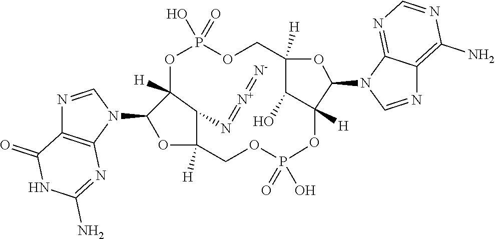 [SMILES: [N-]=[N+]=N[C@H]1[C@H]2OP(=O)(O)OC[C@H]3O[C@@H](n4cnc5c(N)ncnc54)[C@H](OP(=O)(O)OC[C@H]1O[C@H]2n1cnc2c(=O)[nH]c(N)nc21)[C@@H]3O] |
| T4-390 | 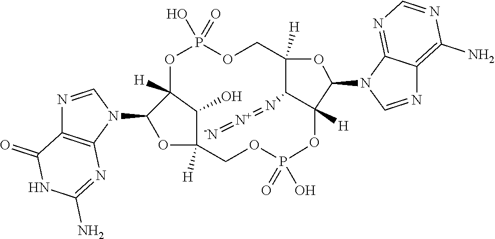 |
| T4-391 | 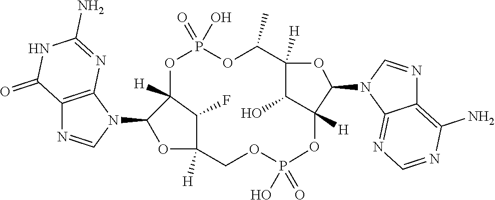 [SMILES: C[C@H]1OP(=O)(O)O[C@@H]2[C@H](F)[C@@H](COP(=O)(O)O[C@@H]3[C@H](O)[C@@H]1O[C@H]3n1cnc3c(N)ncnc31)O[C@H]2n1cnc2c(=O)[nH]c(N)nc21] |
| T4-392 | 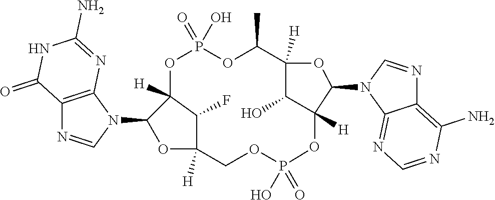 [SMILES: C[C@@H]1OP(=O)(O)O[C@@H]2[C@H](F)[C@@H](COP(=O)(O)O[C@@H]3[C@H](O)[C@@H]1O[C@H]3n1cnc3c(N)ncnc31)O[C@H]2n1cnc2c(=O)[nH]c(N)nc21] |
| T4-393 | 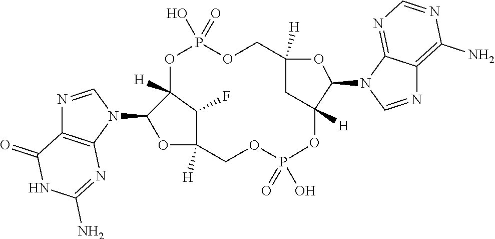 [SMILES: Nc1nc2c(ncn2[C@@H]2O[C@@H]3COP(=O)(O)O[C@@H]4C[C@@H](COP(=O)(O)O[C@@H]2[C@@H]3F)O[C@H]4n2cnc3c(N)ncnc32)c(=O)[nH]1] |
| T4-394 | 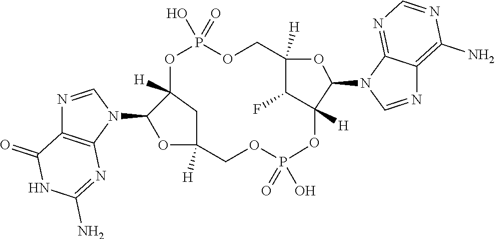 [SMILES: Nc1nc2c(ncn2[C@@H]2O[C@@H]3COP(=O)(O)O[C@@H]4[C@H](F)[C@@H](COP(=O)(O)O[C@@H]2C3)O[C@H]4n2cnc3c(N)ncnc32)c(=O)[nH]1] |
| T4-395 | 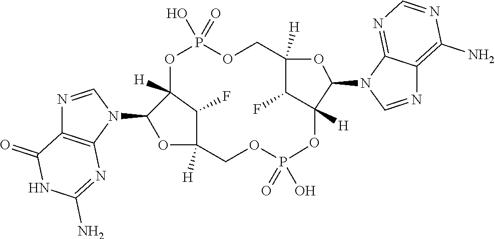 [SMILES: Nc1nc2c(ncn2[C@@H]2O[C@@H]3COP(=O)(O)O[C@@H]4[C@H](F)[C@@H](COP(=O)(O)O[C@@H]2[C@@H]3F)O[C@H]4n2cnc3c(N)ncnc32)c(=O)[nH]1] |
| T4-396 | 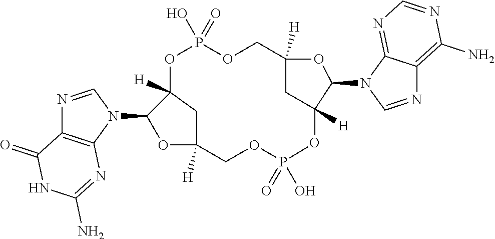 [SMILES: Nc1nc2c(ncn2[C@@H]2O[C@@H]3COP(=O)(O)O[C@@H]4C[C@@H](COP(=O)(O)O[C@@H]2C3)O[C@H]4n2cnc3c(N)ncnc32)c(=O)[nH]1] |
| T4-397 | 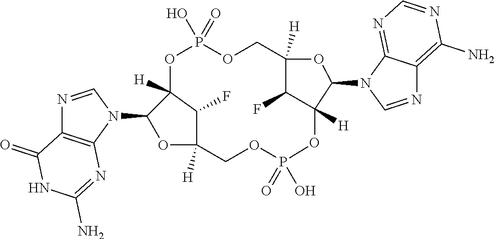 [SMILES: Nc1nc2c(ncn2[C@@H]2O[C@@H]3COP(=O)(O)O[C@@H]4[C@@H](F)[C@@H](COP(=O)(O)O[C@@H]2[C@@H]3F)O[C@H]4n2cnc3c(N)ncnc32)c(=O)[nH]1] |
| T4-398 | 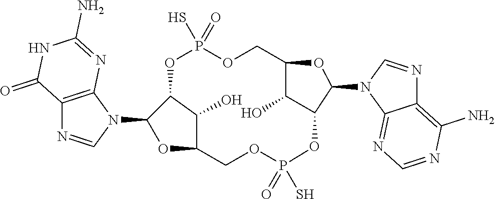 [SMILES: Nc1nc2c(ncn2[C@@H]2O[C@@H]3COP(=O)(S)O[C@@H]4[C@H](O)[C@@H](COP(=O)(S)O[C@@H]2[C@@H]3O)O[C@H]4n2cnc3c(N)ncnc32)c(=O)[nH]1] |
| T4-399 | 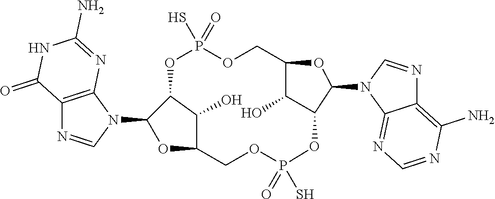 [SMILES: Nc1nc2c(ncn2[C@@H]2O[C@@H]3COP(=O)(S)O[C@@H]4[C@H](O)[C@@H](COP(=O)(S)O[C@@H]2[C@@H]3O)O[C@H]4n2cnc3c(N)ncnc32)c(=O)[nH]1] |
| T4-400 | 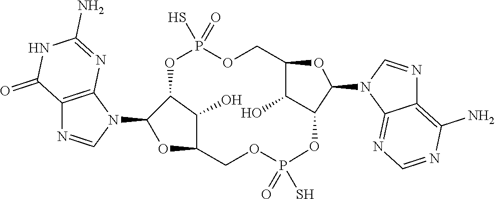 [SMILES: Nc1nc2c(ncn2[C@@H]2O[C@@H]3COP(=O)(S)O[C@@H]4[C@H](O)[C@@H](COP(=O)(S)O[C@@H]2[C@@H]3O)O[C@H]4n2cnc3c(N)ncnc32)c(=O)[nH]1] |
| T4-401 | 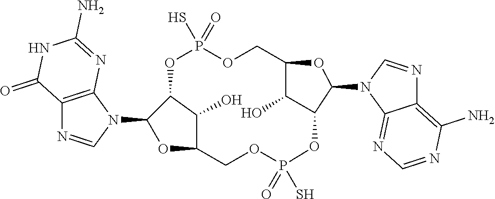 [SMILES: Nc1nc2c(ncn2[C@@H]2O[C@@H]3COP(=O)(S)O[C@@H]4[C@H](O)[C@@H](COP(=O)(S)O[C@@H]2[C@@H]3O)O[C@H]4n2cnc3c(N)ncnc32)c(=O)[nH]1] |
| T4-402 | 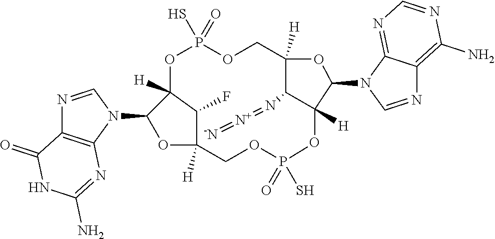 |
| T4-403 | 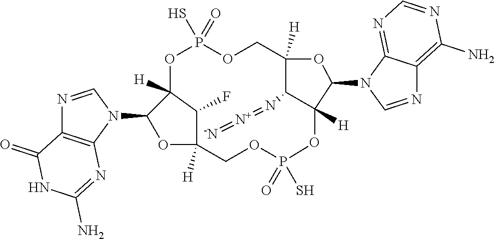 |
| T4-404 | 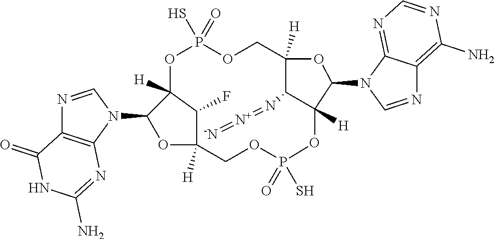 |
| T4-405 | 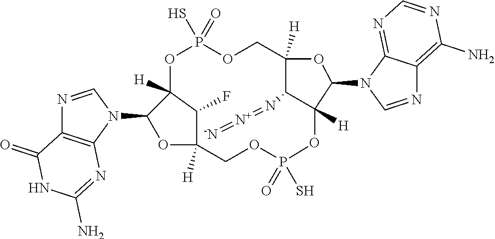 |
| T4-406 | 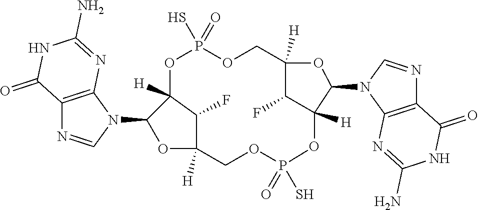 [SMILES: Nc1nc2c(ncn2[C@@H]2O[C@@H]3COP(=O)(S)O[C@@H]4[C@H](F)[C@@H](COP(=O)(S)O[C@@H]2[C@@H]3F)O[C@H]4n2cnc3c(=O)[nH]c(N)nc32)c(=O)[nH]1] |
| T4-407 | 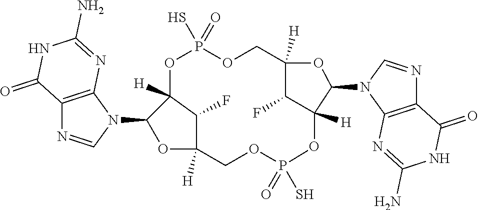 [SMILES: Nc1nc2c(ncn2[C@@H]2O[C@@H]3COP(=O)(S)O[C@@H]4[C@H](F)[C@@H](COP(=O)(S)O[C@@H]2[C@@H]3F)O[C@H]4n2cnc3c(=O)[nH]c(N)nc32)c(=O)[nH]1] |
| T4-408 | 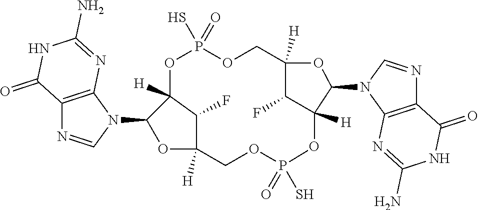 [SMILES: Nc1nc2c(ncn2[C@@H]2O[C@@H]3COP(=O)(S)O[C@@H]4[C@H](F)[C@@H](COP(=O)(S)O[C@@H]2[C@@H]3F)O[C@H]4n2cnc3c(=O)[nH]c(N)nc32)c(=O)[nH]1] |
| T4-409 | 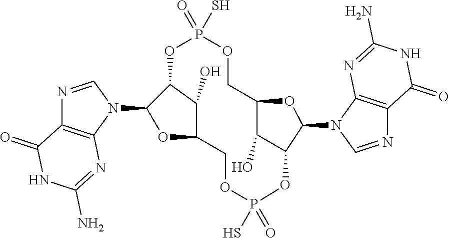 [SMILES: Nc1nc2c(ncn2[C@@H]2O[C@@H]3COP(=O)(S)O[C@@H]4[C@H](O)[C@@H](COP(=O)(S)O[C@@H]2[C@@H]3O)O[C@H]4n2cnc3c(=O)[nH]c(N)nc32)c(=O)[nH]1] |
| T4-410 | 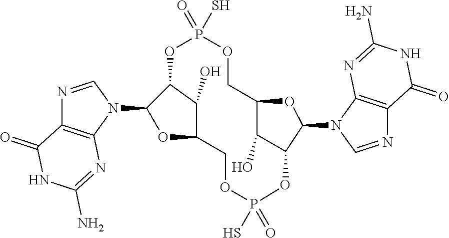 [SMILES: Nc1nc2c(ncn2[C@@H]2O[C@@H]3COP(=O)(S)O[C@@H]4[C@H](O)[C@@H](COP(=O)(S)O[C@@H]2[C@@H]3O)O[C@H]4n2cnc3c(=O)[nH]c(N)nc32)c(=O)[nH]1] |
| T4-411 | 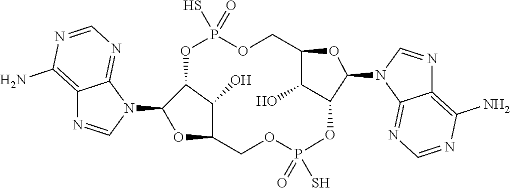 [SMILES: Nc1ncnc2c1ncn2[C@@H]1O[C@@H]2COP(=O)(S)O[C@@H]3[C@H](O)[C@@H](COP(=O)(S)O[C@@H]1[C@@H]2O)O[C@H]3n1cnc2c(N)ncnc21] |
| T4-412 | 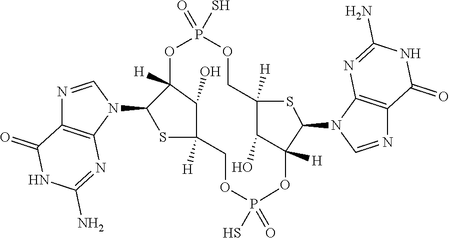 [SMILES: Nc1nc2c(ncn2[C@@H]2S[C@@H]3COP(=O)(S)O[C@@H]4[C@H](O)[C@@H](COP(=O)(S)O[C@@H]2[C@@H]3O)S[C@H]4n2cnc3c(=O)[nH]c(N)nc32)c(=O)[nH]1] |
| T4-413 | 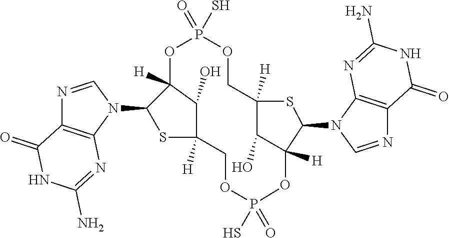 [SMILES: Nc1nc2c(ncn2[C@@H]2S[C@@H]3COP(=O)(S)O[C@@H]4[C@H](O)[C@@H](COP(=O)(S)O[C@@H]2[C@@H]3O)S[C@H]4n2cnc3c(=O)[nH]c(N)nc32)c(=O)[nH]1] |
| T4-414 | 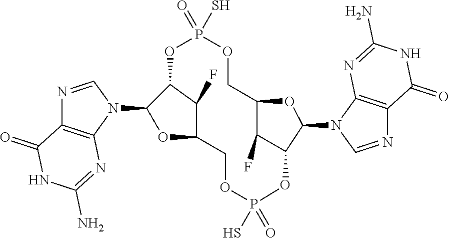 [SMILES: Nc1nc2c(ncn2[C@@H]2O[C@@H]3COP(=O)(S)O[C@@H]4[C@@H](F)[C@@H](COP(=O)(S)O[C@@H]2[C@H]3F)O[C@H]4n2cnc3c(=O)[nH]c(N)nc32)c(=O)[nH]1] |
| T4-415 | 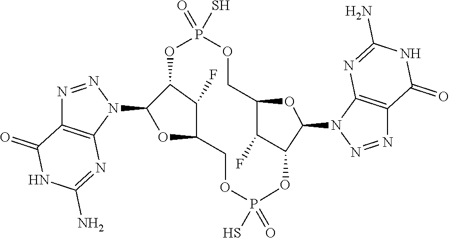 [SMILES: Nc1nc2c(nnn2[C@@H]2O[C@@H]3COP(=O)(S)O[C@@H]4[C@H](F)[C@@H](COP(=O)(S)O[C@@H]2[C@@H]3F)O[C@H]4n2nnc3c(=O)[nH]c(N)nc32)c(=O)[nH]1] |
| T4-416 | 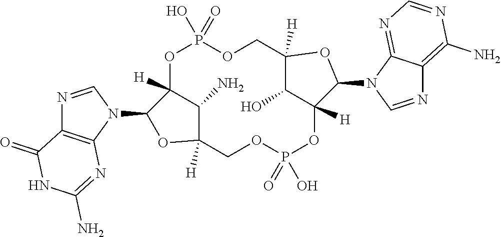 [SMILES: Nc1nc2c(ncn2[C@@H]2O[C@@H]3COP(=O)(O)O[C@@H]4[C@H](O)[C@@H](COP(=O)(O)O[C@@H]2[C@@H]3N)O[C@H]4n2cnc3c(N)ncnc32)c(=O)[nH]1] |
| T4-417 | 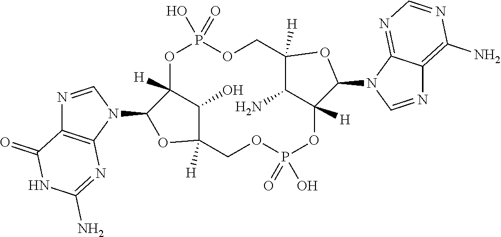 [SMILES: Nc1nc2c(ncn2[C@@H]2O[C@@H]3COP(=O)(O)O[C@@H]4[C@H](N)[C@@H](COP(=O)(O)O[C@@H]2[C@@H]3O)O[C@H]4n2cnc3c(N)ncnc32)c(=O)[nH]1] |
| T4-418 | 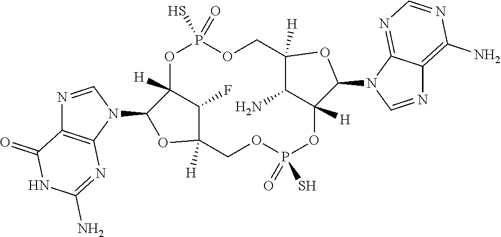 [SMILES: Nc1nc2c(ncn2[C@@H]2O[C@@H]3CO[P@](=O)(S)O[C@@H]4[C@H](N)[C@@H](CO[P@@](=O)(S)O[C@@H]2[C@@H]3F)O[C@H]4n2cnc3c(N)ncnc32)c(=O)[nH]1] |
| T4-419 | 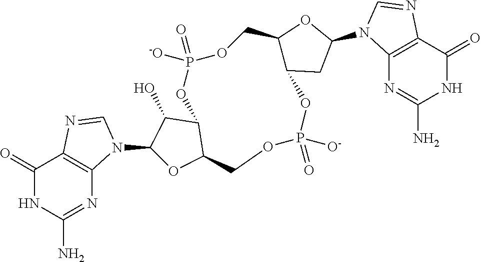 |
| T4-420 | 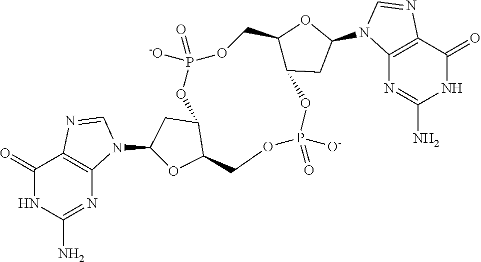 |
| T4-421 | 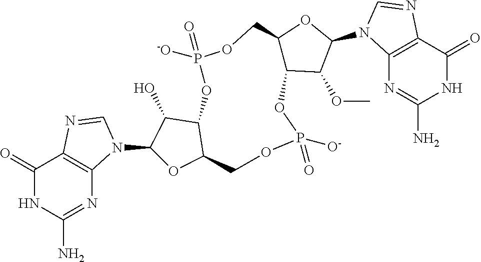 |
| T4-422 | 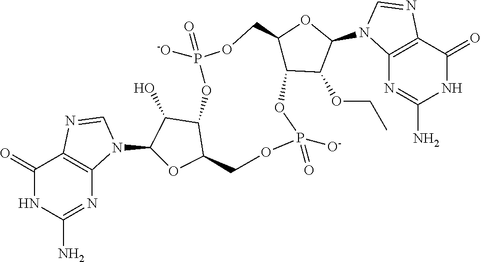 |
| T4-423 | 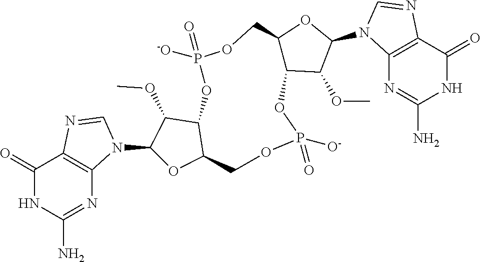 |
| T4-424 | 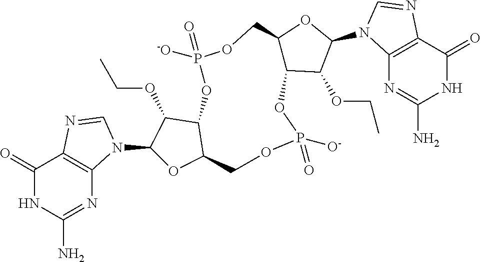 |
| T4-425 | 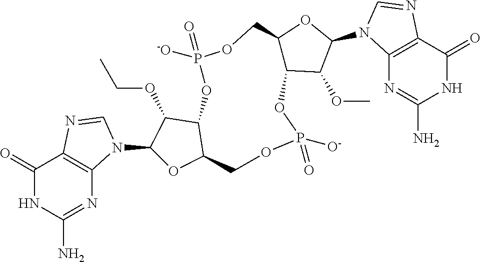 |
| T4-426 | 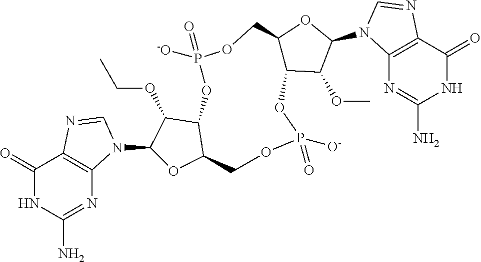 |
| T4-427 | 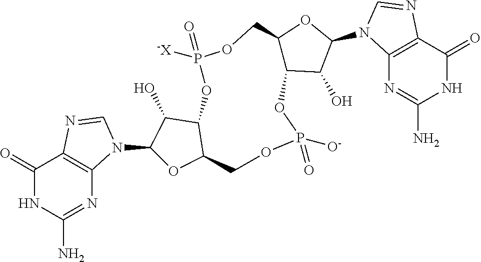 |
| | X = S, Se and BH3 |
| T4-428 | 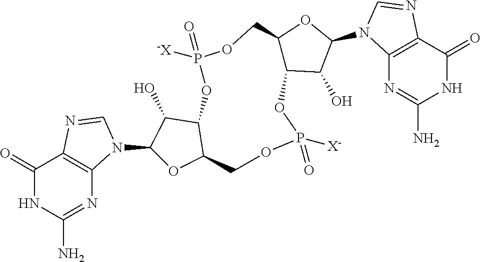 |
| | X = S and Se |
| T4-429 | 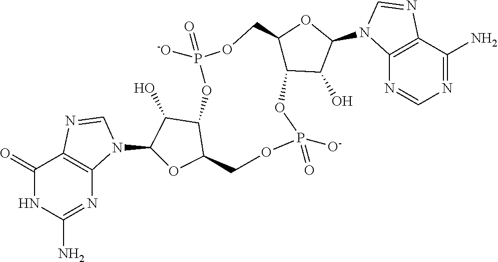 |
| T4-430 | 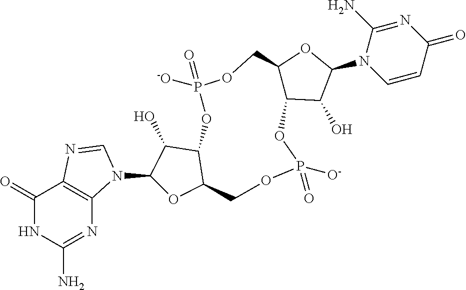 |
| T4-431 | 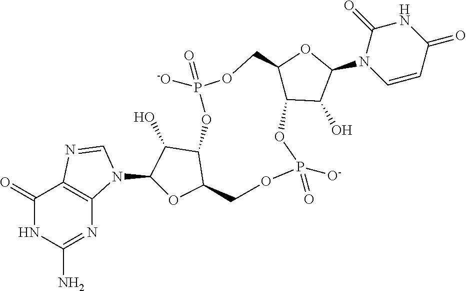 |
| T4-432 | 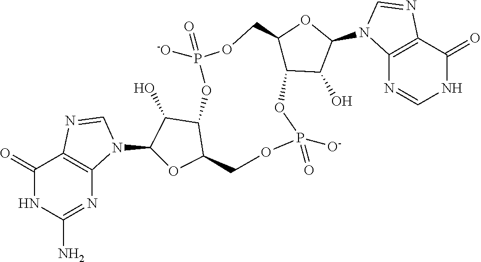 |
| T4-433 | 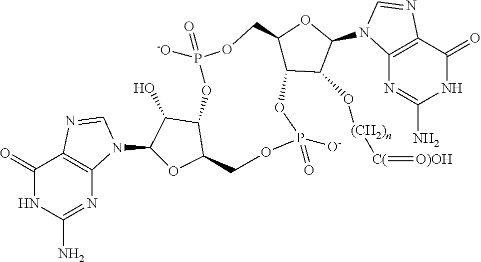 |
| T4-434 | 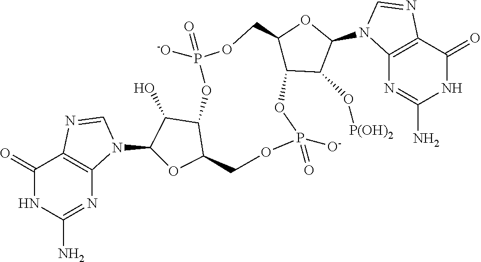 |
| T4-435 | 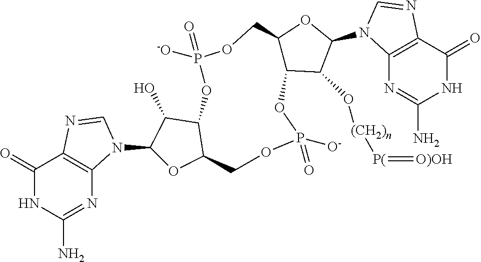 |
| T4-436 | 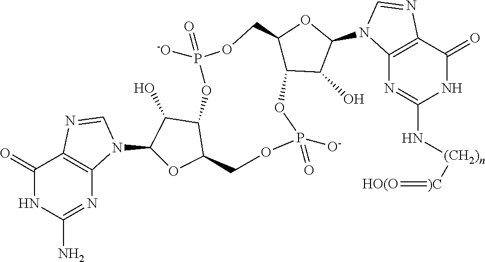 |
| T4-437 | 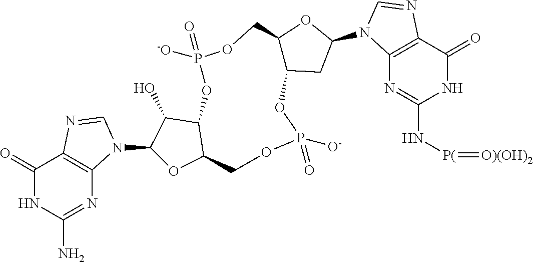 |
| T4-438 | 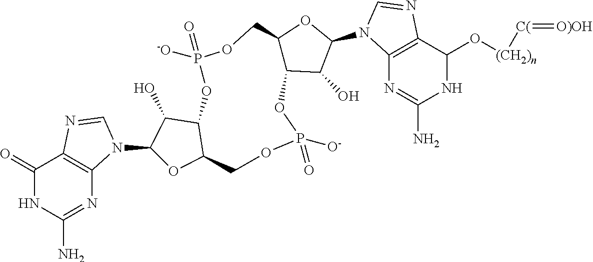 |
| T4-439 | 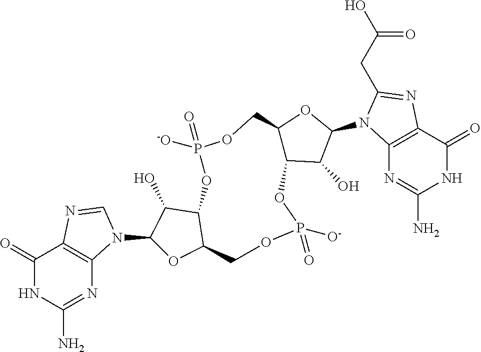 |
| T4-440 | 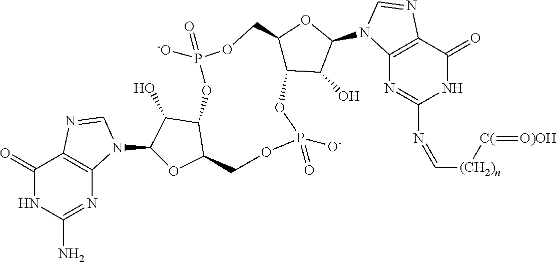 |
| T4-441 | 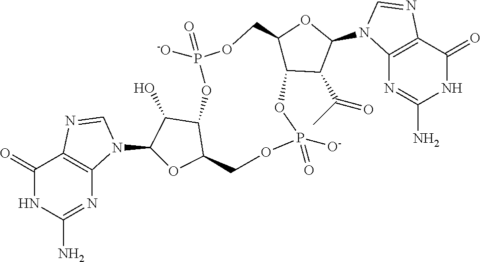 |
| T4-442 | 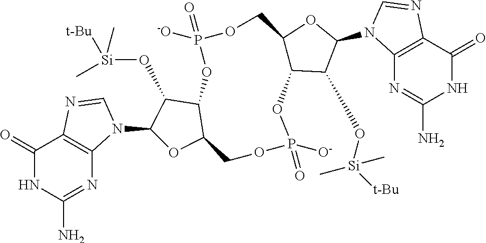 |
| T4-443 | 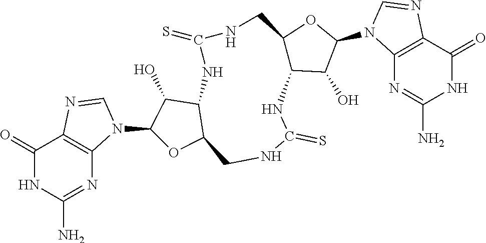 [SMILES: Nc1nc2c(ncn2[C@@H]2O[C@@H]3CNC(=S)N[C@H]4[C@@H](O)[C@H](n5cnc6c(=O)[nH]c(N)nc65)O[C@@H]4CNC(=S)N[C@H]3[C@H]2O)c(=O)[nH]1] |
| T4-444 | 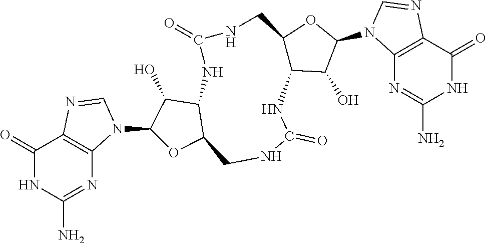 [SMILES: Nc1nc2c(ncn2[C@@H]2O[C@@H]3CNC(=O)N[C@H]4[C@@H](O)[C@H](n5cnc6c(=O)[nH]c(N)nc65)O[C@@H]4CNC(=O)N[C@H]3[C@H]2O)c(=O)[nH]1] |
| T4-445 | 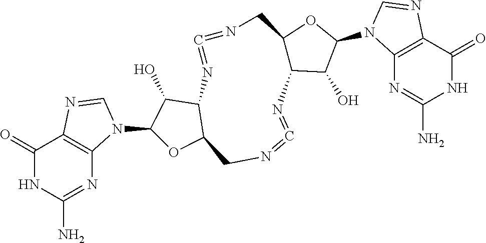 [SMILES: Nc1nc2c(ncn2[C@@H]2O[C@@H]3CN=C=N[C@H]4[C@@H](O)[C@H](n5cnc6c(=O)[nH]c(N)nc65)O[C@@H]4CN=C=N[C@H]3[C@H]2O)c(=O)[nH]1] |
| T4-446 | 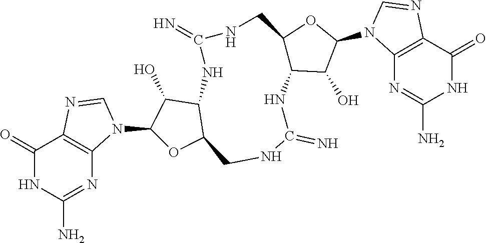 [SMILES: N=C1NC[C@H]2O[C@@H](n3cnc4c(=O)[nH]c(N)nc43)[C@H](O)[C@@H]2NC(=N)NC[C@H]2O[C@@H](n3cnc4c(=O)[nH]c(N)nc43)[C@H](O)[C@@H]2N1] |
| T4-447 | 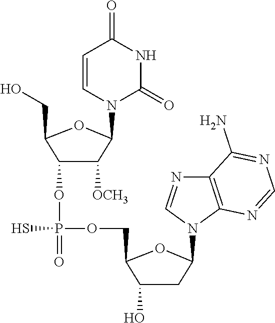 [SMILES: CO[C@@H]1[C@H](O[P@@](=O)(S)OC[C@H]2O[C@@H](n3cnc4c(N)ncnc43)C[C@@H]2O)[C@@H](CO)O[C@H]1n1ccc(=O)[nH]c1=O] |
| T4-448 | 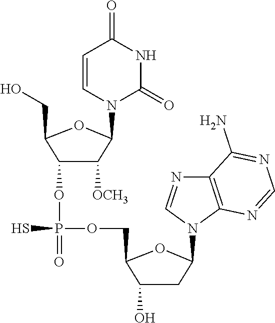 [SMILES: CO[C@@H]1[C@H](O[P@](=O)(S)OC[C@H]2O[C@@H](n3cnc4c(N)ncnc43)C[C@@H]2O)[C@@H](CO)O[C@H]1n1ccc(=O)[nH]c1=O] |
| T4-449 | 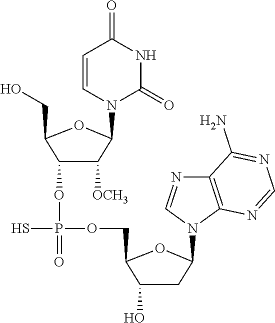 [SMILES: CO[C@@H]1[C@H](OP(=O)(S)OC[C@H]2O[C@@H](n3cnc4c(N)ncnc43)C[C@@H]2O)[C@@H](CO)O[C@H]1n1ccc(=O)[nH]c1=O] |
| T4-450 | 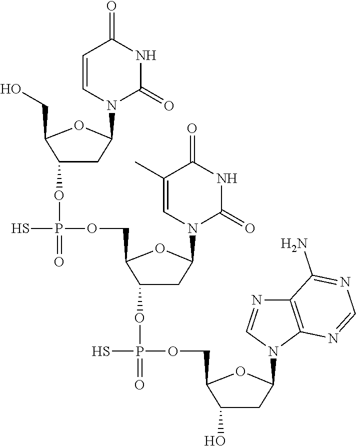 [SMILES: Cc1cn([C@H]2C[C@H](OP(=O)(S)OC[C@H]3O[C@@H](n4cnc5c(N)ncnc54)C[C@@H]3O)[C@@H](COP(=O)(S)O[C@H]3C[C@H](n4ccc(=O)[nH]c4=O)O[C@@H]3CO)O2)c(=O)[nH]c1=O] |
| T4-451 | 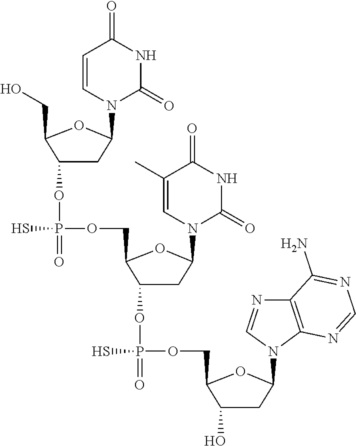 [SMILES: Cc1cn([C@H]2C[C@H](O[P@@](=O)(S)OC[C@H]3O[C@@H](n4cnc5c(N)ncnc54)C[C@@H]3O)[C@@H](CO[P@](=O)(S)O[C@H]3C[C@H](n4ccc(=O)[nH]c4=O)O[C@@H]3CO)O2)c(=O)[nH]c1=O] |
| T4-452 | 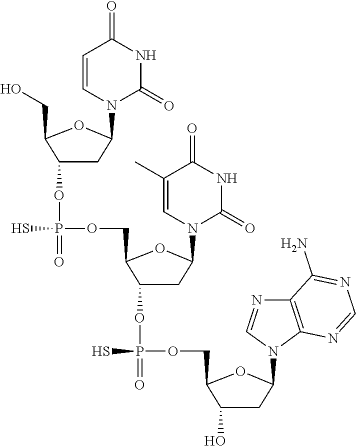 [SMILES: Cc1cn([C@H]2C[C@H](O[P@](=O)(S)OC[C@H]3O[C@@H](n4cnc5c(N)ncnc54)C[C@@H]3O)[C@@H](CO[P@](=O)(S)O[C@H]3C[C@H](n4ccc(=O)[nH]c4=O)O[C@@H]3CO)O2)c(=O)[nH]c1=O] |
| T4-453 | 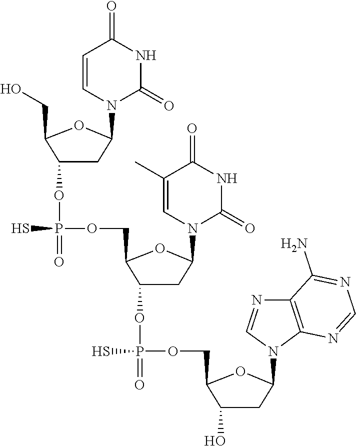 [SMILES: Cc1cn([C@H]2C[C@H](O[P@@](=O)(S)OC[C@H]3O[C@@H](n4cnc5c(N)ncnc54)C[C@@H]3O)[C@@H](CO[P@@](=O)(S)O[C@H]3C[C@H](n4ccc(=O)[nH]c4=O)O[C@@H]3CO)O2)c(=O)[nH]c1=O] |
| T4-454 | 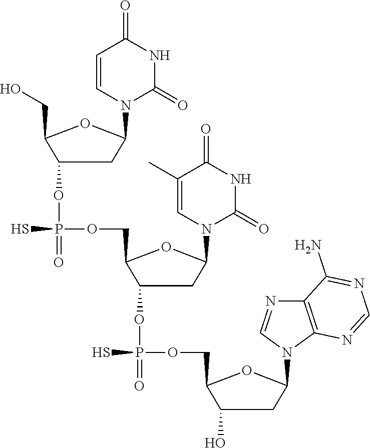 [SMILES: Cc1cn([C@H]2C[C@H](O[P@](=O)(S)OC[C@H]3O[C@@H](n4cnc5c(N)ncnc54)C[C@@H]3O)[C@@H](CO[P@@](=O)(S)O[C@H]3C[C@H](n4ccc(=O)[nH]c4=O)O[C@@H]3CO)O2)c(=O)[nH]c1=O] |
| T4-455 | 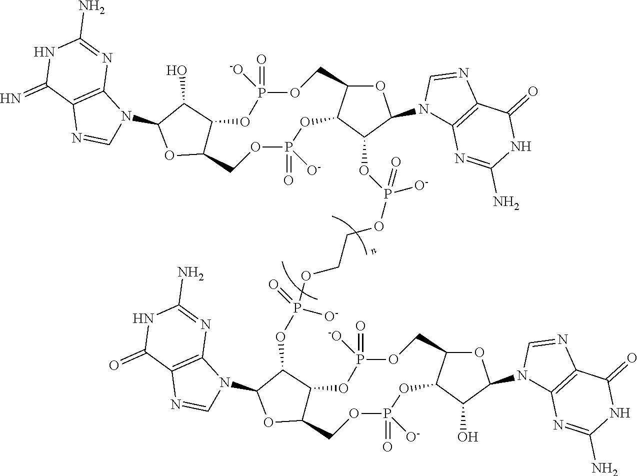 |
| | n is an integer selected from 4 to 18 |
| T4-456 | 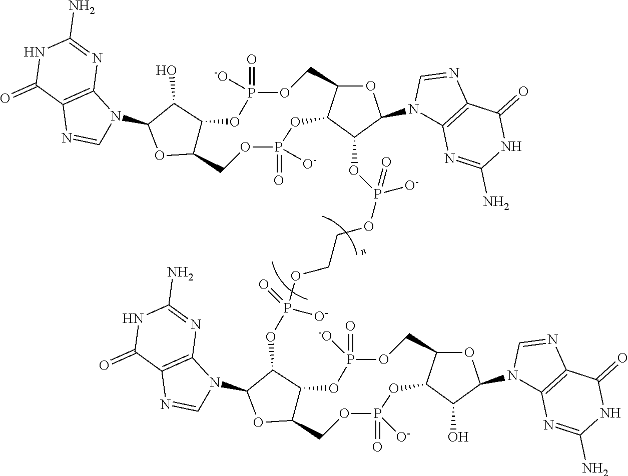 |
| | n is an integer selected from 4 to 18 |

### Example Synthesis of Compounds of Formula (A)

Compounds of Formula (A) were made according to the synthetic description in WO2016145102.

Specifically, (2R,3R,3aR,5R,7aR,9R,10R,10aR,12R,14aR)-2,9-bis(6-amino-9H-purin-9-yl)-3,10-difluorooctahydro-2H,7H-difuro[3,2-d:3′,2′-j][1,3,7,9]tetraoxa[2,8]diphosphacyclododecine-5,12-bis(thiolate) 5,12-dioxide (T1-1), and (2R,3R,3aR,5R,7aR,9R,10R,10aR,12S,14aR)-2,9-bis(6-amino-9H-purin-9-yl)-3,10-difluorooctahydro-2H,7H-difuro[3,2-d:3′,2′-j][1,3,7,9]tetraoxa[2,8]diphosphacyclododecine-5,12-bis(thiolate) 5,12-dioxide (T1-6) were synthesized according to the scheme below:

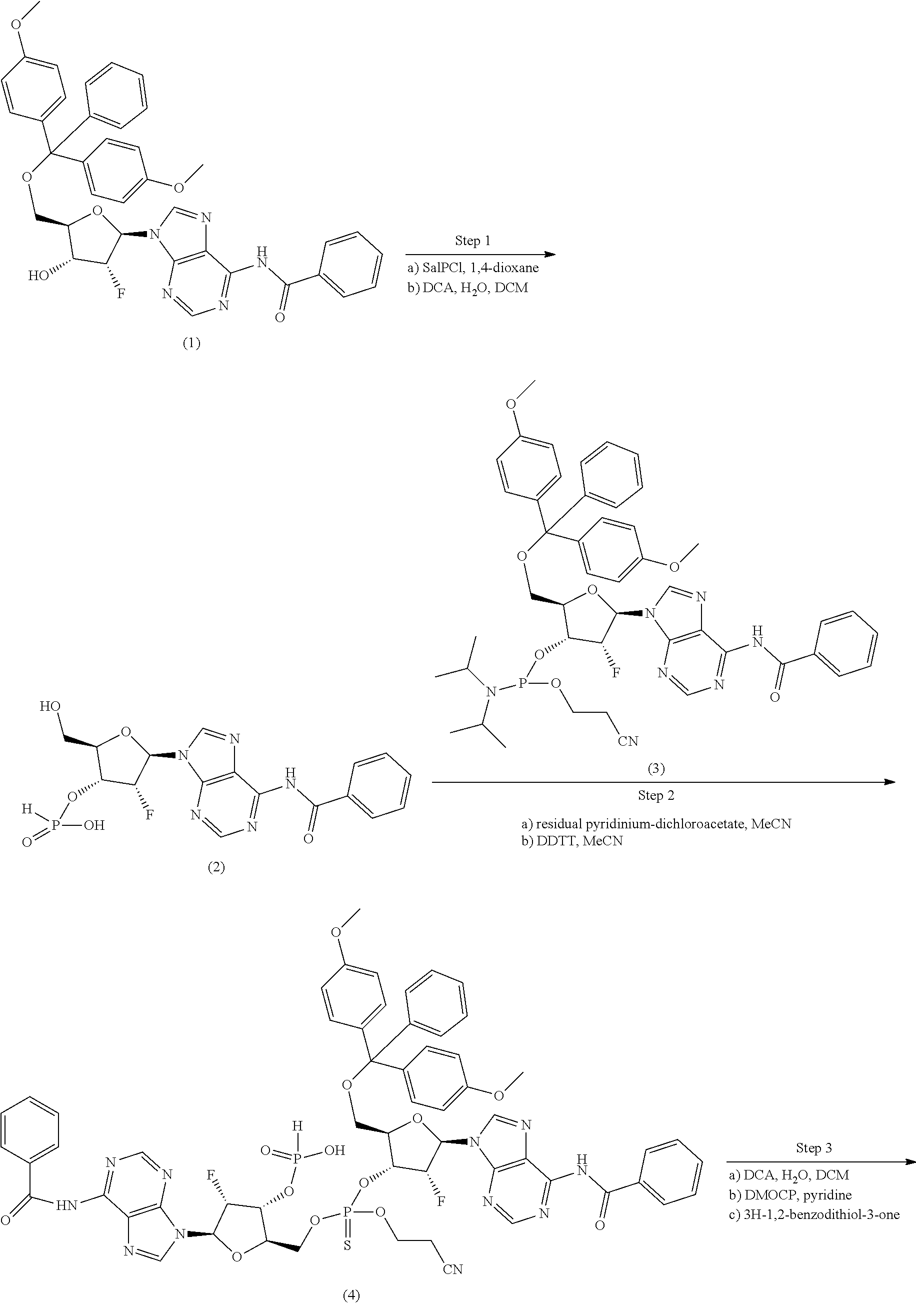 [SMILES: COc1ccc(C(OC[C@H]2O[C@@H](n3cnc4c(NC(=O)c5ccccc5)ncnc43)[C@H](F)[C@@H]2O)(c2ccccc2)c2ccc(OC)cc2)cc1 ; O=C(Nc1ncnc2c1ncn2[C@@H]1O[C@H](CO)[C@@H](O[PH](=O)O)[C@H]1F)c1ccccc1 ; COc1ccc(C(OC[C@H]2O[C@@H](n3cnc4c(NC(=O)c5ccccc5)ncnc43)[C@H](F)[C@@H]2OP(OCCC#N)N(C(C)C)C(C)C)(c2ccccc2)c2ccc(OC)cc2)cc1 ; COc1ccc(C(OC[C@H]2O[C@@H](n3cnc4c(NC(=O)c5ccccc5)ncnc43)[C@H](F)[C@@H]2OP(=S)(OCCC#N)OC[C@H]2O[C@@H](n3cnc4c(NC(=O)c5ccccc5)ncnc43)[C@H](F)[C@@H]2O[PH](=O)O)(c2ccccc2)c2ccc(OC)cc2)cc1]

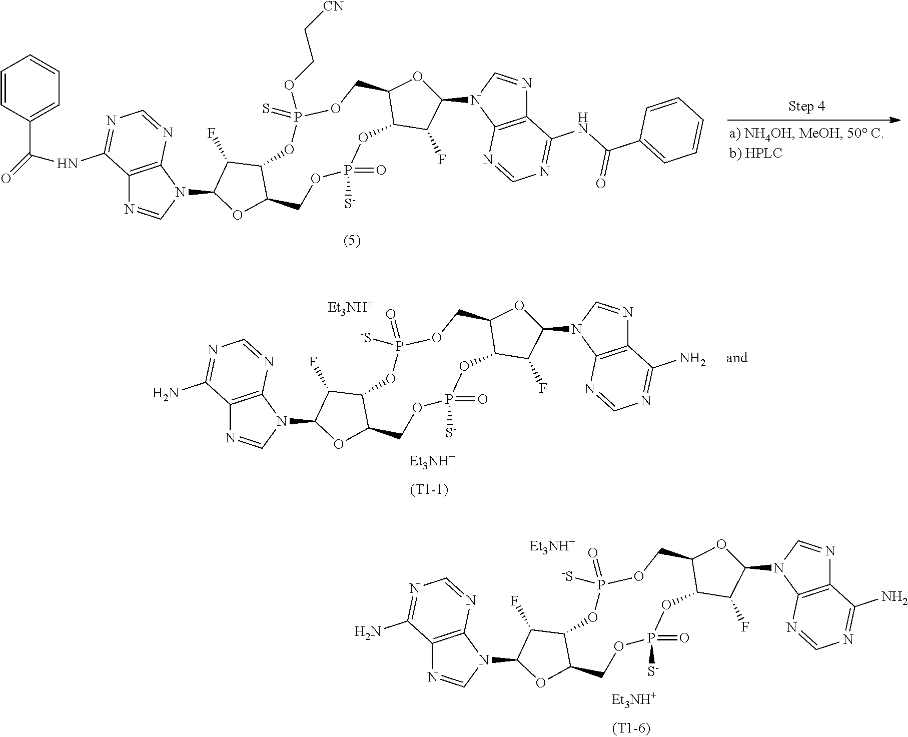

Step 1:

Preparation of (2R,3R,4R,5R)-5-(6-benzamido-9H-purin-9-yl)-4-fluoro-2-(hydroxymethyl)tetrahydrofuran-3-yl hydrogen phosphonate (2): To a solution of N-(9-((2R,3R,4R,5R)-5-((bis(4-methoxyphenyl)(phenyl)methoxy)methyl)-3-fluoro-4-hydroxytetrahydrofuran-2-yl)-9-H-purin-6-yl)benzamide (1, 2.0 g, 3.0 mmol, ChemGenes) in 1,4-dioxane (25 mL) and pyridine (8 mL) was added a solution of 2-Chloro-1,3,2-benzodioxaphosphorin-4-one (SalPCI) (0.84 g, 4.1 mmol) in 1,4-dioxane (12 mL). After 30 min, to the stirred reaction mixture at room temperature was introduced water (4 mL), and the resulting mixture was poured into a 1N aqueous NaHCO₃ solution (100 mL). This aqueous mixture was extracted with EtOAc (3×100 mL) and the layers were partitioned. The EtOAc extracts were combined and concentrated to dryness in vacuo as a colorless foam. The colorless foam was dissolved in CH₂Cl₂ (30 mL) to give a colorless solution. To this solution was added water (0.5 mL) and a 6% (v/v) solution of dichloroacetic acid (DCA) in CH₂Cl₂ (30 mL). After ten min of stirring at room temperature, to the red solution was charged pyridine (3.5 mL). The resulting white mixture was concentrated in vacuo and water was removed as an azeotrope after concentration with MeCN (30 mL). This azeotrope process was repeated two more times with MeCN (30 mL). On the last evaporation, the resulting white slurry of compound 2 was left in MeCN (15 mL).

Step 2:

Preparation of (2R,3R,4R,5R)-5-(6-benzamido-9H-purin-9-yl)-2-((((((2R,3R,4R,5R)-5-(6-benzamido-9H-purin-9-yl)-2-((bis(4-methoxyphenyl)(phenyl)methoxy)methyl)-4-fluorotetrahydrofuran-3-yl)oxy)(2-cyanoethoxy)phosphorothioyl)oxy)methyl)-4-fluorotetrahydrofuran-3-yl hydrogenphosphonate (4): To a solution of (2R,3R,4R,5R)-5-(6-benzamido-9H-purin-9-yl)-2-((bis(4-methoxyphenyl)(phenyl)methoxy)methyl)-4-fluorotetrahydrofuran-3-yl (2-cyanoethyl) diisopropylphosphoramidite (3, 2.5 g, 2.9 mmol, ChemGenes) in MeCN (20 mL) was dried through concentration in vacuo. This process was repeated two more times to remove water as an azeotrope. On the last azeotrope, to the solution of compound 3 in MeCN (7 mL) was introduced ten 3 Å molecular sieves and the solution was stored under an atmosphere of nitrogen. To a stirred mixture of compound 2 with residual pyridin-1-ium dichloroacetate in MeCN (15 mL) was added the solution of compound 3 in MeCN (7 mL). After five min, to the stirred mixture was added 3-((dimethylamino-methylidene)amino)-3H-1,2,4-dithiazole-3-thione (DDTT) (650 mg, 3.2 mmol). After 30 min, the yellow mixture was concentrated in vacuo to give compound 4 as a yellow oil.

Step 3:

Preparation of N,N′-(((2R,3R,3aR,7aR,9R,10R,10aR,12R,14aR)-5-(2-cyanoethoxy)-3,10-difluoro-12-mercapto-12-oxido-5-sulfidooctahydro-2H,7H-difuro[3,2-d:3′,2′-j][1,3,7,9]tetraoxa[2,8]diphosphacyclododecine-2,9-diyl)bis(9H-purine-9,6-diyl))dibenzamide(5): To a solution of compound 4 in CH₂Cl₂ (60 mL) were added water (0.35 mL) and a 6% (v/v) solution of dichloroacetic acid (DCA) in CH₂Cl₂ (60 mL). After ten min at room temperature, to the red solution was introduced pyridine (20 mL). The resulting yellow mixture was concentrated in vacuo until approximately 20 mL of the yellow mixture remained. To the yellow mixture was introduced pyridine (20 mL) and the mixture was concentrated in vacuo until approximately 20 mL of the yellow mixture remained. To the yellow mixture was added pyridine (30 mL) and the mixture was concentrated in vacuo until approximately 30 mL of the yellow mixture remained. To the stirred yellow mixture in pyridine (30 mL) was added 2-chloro-5,5-dimethyl-1,3,2-dioxaphosphorinane-2-oxide (DMOCP) (1.6 g, 8.4 mmol). After seven min, to the dark orange solution was added water (1.4 mL), followed immediately by the introduction of 3H-1,2-benzodithiol-3-one (0.71 mg, 4.2 mmol). After five min, the dark orange solution was poured into a 1N aqueous NaHCO₃ solution (400 mL). After ten min, the biphasic mixture was extracted with EtOAc (200 mL) and diethyl ether (200 mL). After separation, the aqueous layer was back extracted with EtOAc (200 mL) and diethyl ether (200 mL). The organic extracts were combined and concentrated in vacuo. To the concentrated yellow oil was added toluene (75 mL) and the mixture was evaporated in vacuo to remove residual pyridine. This procedure was repeated twice with toluene (75 mL). The resulting oil was purified by silica gel chromatography (0% to 10% MeOH in CH₂Cl₂) to provide compound 5 (67 mg, 2.5% yield) as an orange oil.

Step 4:

Preparation of Compound (T1-1): To a stirred solution of compound 5 (65 mg, 0.07 mmol) in MeOH (0.9 mL) was added aqueous ammonium hydroxide (0.9 mL) and the orange slurry was heated at 50° C. After two hours, the orange solution was allowed to cool and concentrated in vacuo. The orange residue was purified by reverse phase silica gel chromatography (0% to 30% MeCN in 10 mM aqueous Triethylammonium acetate (TEAA) to obtain Compound (T1-1) (18 mg, 38% yield) as a white mono-triethylammonium salt after lyophilization. LCMS-ESI: 693.25 [M−H]− (calculated for C₂₀H₂₂F₂N₁₀O₈P₂S₂: 694.305); Rt: 16.698′ min by HPLC conditions (10 mM TEAA, 2% to 20%); Rt: 20.026′. min by LCMS conditions (20 mM NH₄OAc, 2% to 20%). ¹H NMR (400 MHz, 45° C., D₂O) δ 8.44 (s, 2H), 8.24 (s, 2H), 6.52 (d, J=16.4 Hz, 2H), 5.80 (d, J=3.6 Hz, 1H), 5.67 (d, J=4.0 Hz, 1H), 5.37-5.26 (m, 2H), 4.77-4.65 (m, 4H), 4.22 (dd, J=11.4 Hz, 6.0 Hz, 2H), 3.34 (q, J=7.0 Hz, 6H), 1.43 (t, J=7.0 Hz, 9H). ¹⁹F NMR (400 MHz, 45° C., D₂O) δ −200.74 to −200.98 (m). ³¹P NMR (45° C., D₂O) δ 54.46.

The stereochemistry of this compound, as depicted was confirmed by the co-crystal structure bound to wild type STING protein.

The Rp,Sp isomer was also isolated after purification in the reverse phase chromatography step, to provide Compound (T1-6) as the bistriethylammonium salt after lyophilization. LCMS-ESI: 693.30 [M−H]− (calculated for C₂₀H₂₂F₂N₁₀O₈P₂S₂: 694.05); Rt 13.830 min by HPLC conditions (10 mM TEAA, 2% to 20%). Rt 15.032 min by LCMS conditions (20 mM NH₄OAc, 2% to 20%). ¹H NMR. (400 MHz, 45° C., D₂O) δ 8.65 (s, 1H), 8.50 (s, 1H), 8.34 (s, 1H), 8.26 (s, 1H), 6.58 (dd, J=16.4, 2.8 Hz, 2H), 6.00 (dd, J=51.2, 3.6 Hz, 1H), 5.69 (dd, J=51.2, 3.8 Hz, 1H), 5.32-5.15 (m, 2H), 4.77-4.67 (m, 3H), 4.61 (d, J=12.4 Hz, 1H), 4.25 (dd, J=11.8, 4.2 Hz, 2H), 3.33 (q, J=7.2 Hz, 12H), 1.43 (t, J=7.2 Hz, 18H). ¹⁹F NMR (400 MHz, 45° C., D₂O) δ −200.75 to −201.31 (m). ³¹P NMR (45° C., D₂O) δ 54.69, 54.64.

### Example Synthesis of Compounds of Formula (B)

Compounds of Formula (B) were made according to the synthetic description in WO2014189805.

Specifically, Compound (T1-2),

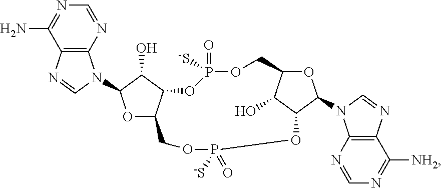

was synthesized according to the scheme below:

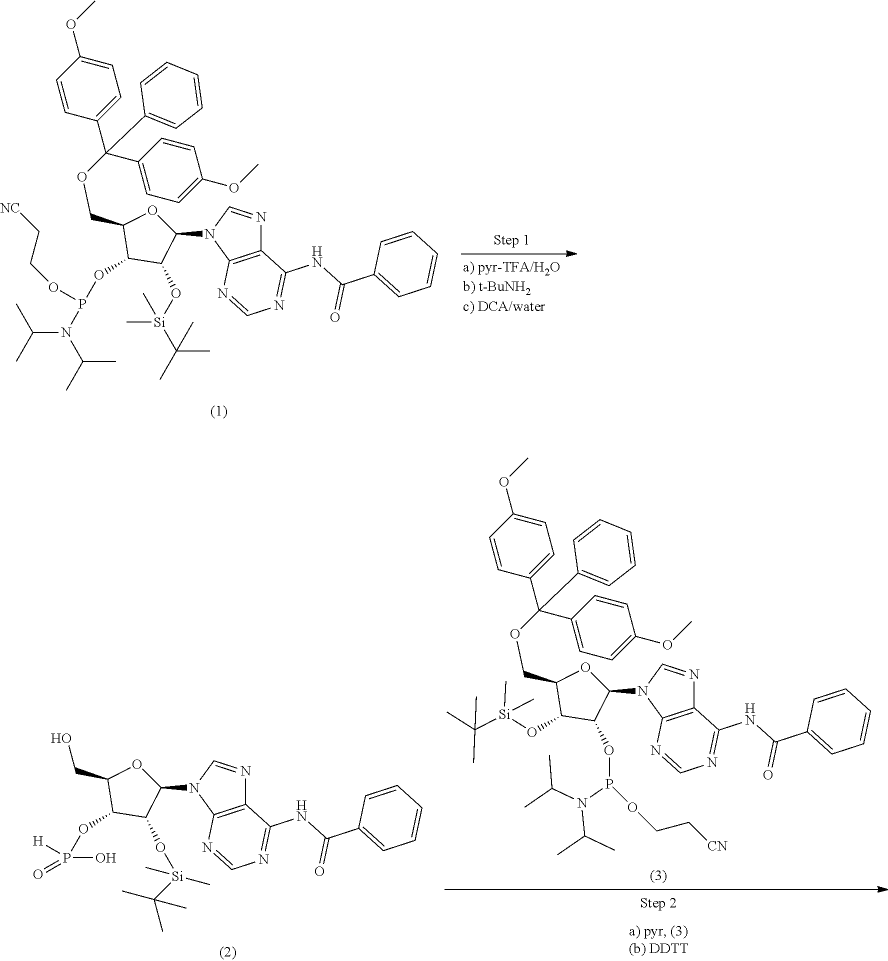 [SMILES: COc1ccc(C(OC[C@H]2O[C@@H](n3cnc4c(NC(=O)c5ccccc5)ncnc43)[C@H](O[Si](C)(C)C(C)(C)C)[C@@H]2OP(OCCC#N)N(C(C)C)C(C)C)(c2ccccc2)c2ccc(OC)cc2)cc1 ; CC(C)(C)[Si](C)(C)O[C@@H]1[C@H](O[PH](=O)O)[C@@H](CO)O[C@H]1n1cnc2c(NC(=O)c3ccccc3)ncnc21 ; COc1ccc(C(OC[C@H]2O[C@@H](n3cnc4c(NC(=O)c5ccccc5)ncnc43)[C@H](OP(OCCC#N)N(C(C)C)C(C)C)[C@@H]2O[Si](C)(C)C(C)(C)C)(c2ccccc2)c2ccc(OC)cc2)cc1]

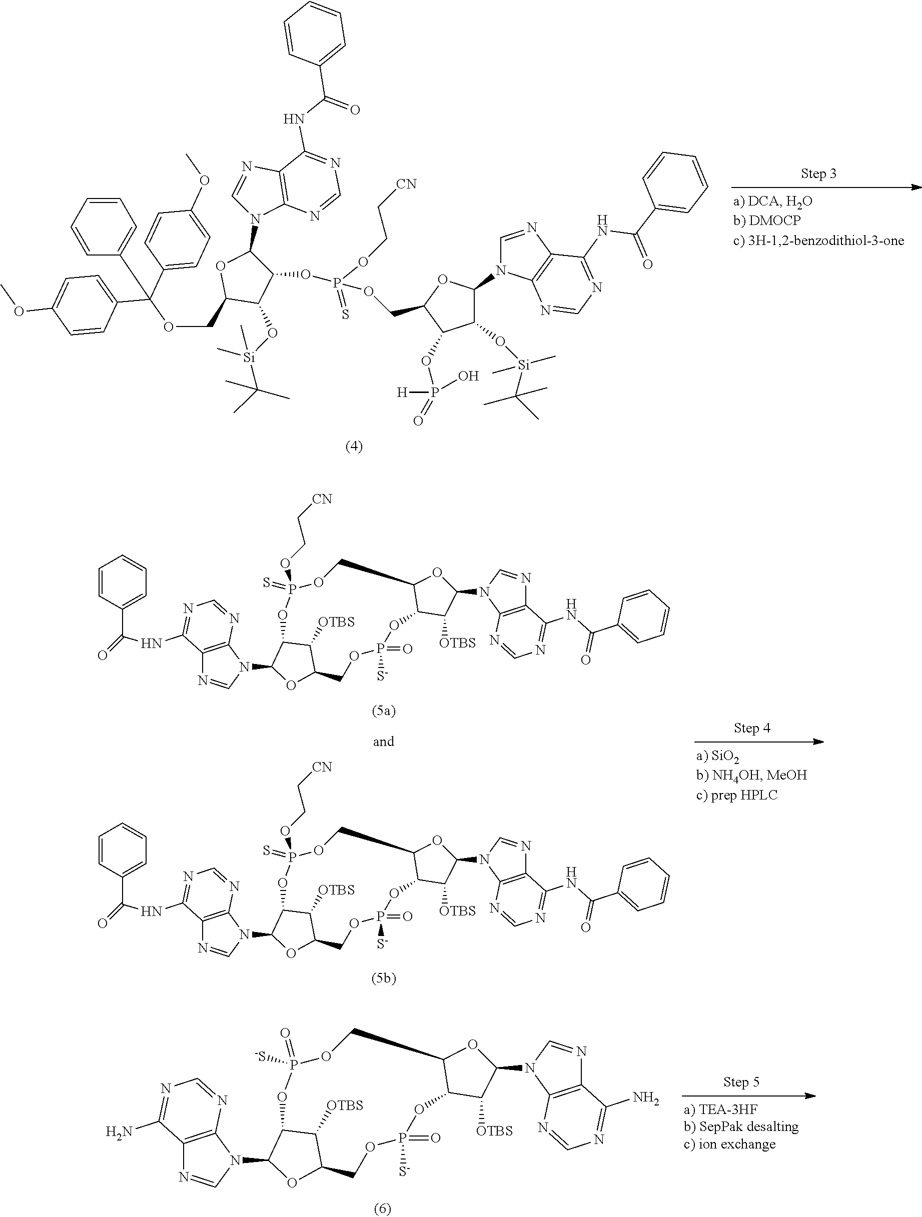

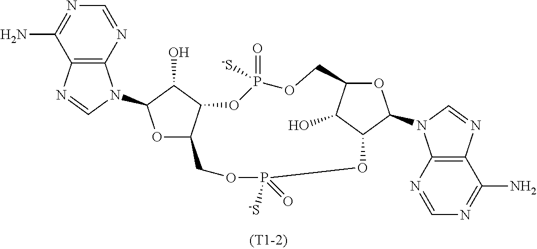

To a solution of 5 g (5.15 mmol) N-benzoyl-5′-O-(4, 4′-dimethoxytrityl)-2′-O-tert-butyldimethylsilyl-3′-O-[(2-cyanoethyl)-ich N-diisopropylaminophinyl]adenosine (1) in 25 ml acetonitrile was added 0.18 ml (10 mmole) water and 1.20 g (6.2 mmole) pyridinium trifluoroacetate. After 5 minutes stirring at room temperature 25 ml tertbutylamine was added and the reaction stirred for 15 minutes at room temperature. The solvents were removed under reduced pressure to give (2R,3R,4R,5R)-5-(6-benzamido-9H-purin-9-yl)-2-((bis(4-methoxyphenyl)(phenyl)methoxy)methyl)-4-((tert-butyldimethylsilyl)oxy)tetrahydrofuran-3-yl hydrogen phosphonate as a foam which was then coevaporated with acetonitrile (2×50 ml), then dissolved in 60 ml dichloromethane. To this solution was added water (0.9 ml, 50 mmole) and 60 ml of 6% (v/v) dichloroacetic acid (44 mmol) in dichloromethane. After 10 minutes at room temperature the reaction was quenched by the addition of pyridine (7.0 ml, 87 mmol), and concentrated to an oil which was dried by three co-evaporations with 40 ml anhydrous acetonitrile giving (2) in a volume of 12 ml.

N-benzoyl-5′-O-(4, 4′-dimethoxytrityl)-3′-O-tert-butyldimethylsilyl-2′-O-[(2-cyanoethyl)-N, N-diisopropylaminophinyl]adenosine ((3), 6.4 g, 6.6 mmole) was dissolved in 40 ml anhydrous acetonitrile and dried by three co-evaporations with 40 ml anhydrous acetonitrile, the last time leaving 20 ml. 3 Å molecular sieves were added and the solution stored under argon until used. Azeo dried (3) (6.4 g, 6.6 mmole) in 20 ml acetonitrile was added via syringe to a solution of (2) (5.15 mmol) in 12 ml of anhydrous acetonitrile. After 5 minutes stirring at room temperature, 1.14 g (5.6 mmol) of 3-((N,N-dimethylaminomethylidene) amino)-3H-1,2,4-dithiazole-5-thione (DDTT) was added and the reaction stirred for 30 minutes at room temperature. The reaction was concentrated and the residual oil dissolved in 80 ml dichloromethane. Water (0.9 ml, 50 mmol) and 80 ml of 6% (v/v) dichloroacetic acid (58 mmol) in dichloromethane was added, and the reaction stirred for 10 minutes at room temperature. 50 ml pyridine was added to quench the dichloroacetic acid. The solvents were removed under reduced pressure to give crude (2R,3R,4R,5R)-5-(6-benzamido-9H-purin-9-yl)-2-((((((2R,3R,4R,5R)-2-(6-benzamido-9H-purin-9-yl)-4-((tert-butyldimethylsilyl)oxy)-5-(hydroxymethyl)tetrahydrofuran-3-yl)oxy) (2-cyanoethoxy)phosphorothioyl)oxy)methyl)-4-((tert-butyldimethylsilyl)oxy)tetrahydrofuran-3-yl hydrogen phosphonate as a solid, which was then dissolved in 150 ml dry pyridine and concentrated down to a volume of approximately 100 ml. 2-chloro-5, 5-dimethyl-1,3,2-dioxaphosphorinane-2-oxide (DMOCP, 3.44 g, 18 mmole) was then added and the reaction stirred for 5 minutes at room temperature. 3.2 ml water was added immediately followed by addition of 3-H-1,2-benzodithiol-3-one (1.3 g, 7.7 mmole), and the reaction stirred for 5 minutes at room temperature. The reaction mix was then poured into 700 ml water containing 20 g NaHCO₃ and stirred for 5 minutes at room temperature, then poured into a separatory funnel and extracted with 800 ml 1:1ethyl acetate:diethyl ether. The aqueous layer was extracted again with 600 ml 1:1 ethyl acetate:diethyl ether. The organic layers were combined and concentrated under reduced pressure to yield approximately 11 g of an oil containing diastereoisomers (5a) and (5b). The crude mixture above was dissolved in dichloromethane and applied to a 250 g silica column. The desired diastereoisomers were eluted from the column using a gradient of ethanol in dichloromethane (0-10%). Fractions containing the desired diastereoisomers (5a) and (5b) were combined and concentrated, giving 2.26 g of approximately 50% (5a) and 50% (5b).

2.26 g of crude (5a) and (5b) from the silica gel column was transferred to a thick-walled glass pressure tube. 60 ml methanol and 60 ml concentrated aqueous ammonia was added and the tube was heated with stirring in an oil bath at 500C for 16 h. The reaction mixture was cooled to near ambient temperature, sparged with a stream of nitrogen gas for 30 minutes, and then transferred to a large round bottom flask. Most of the volatiles were removed under reduced pressure with caution so as to avoid foaming and bumping. If water was still present the residue was frozen and lyophilized to dryness. The lyophilized crude mixture was taken up in approximately 50 ml of CH₃CN/10 mM aqueous triethylammonium acetate (60/40). After 0.45 micron PTFE filtration, 4-5 ml sample portions were applied to a C-18 Dynamax column (40×250 mm). Elution was performed with a gradient of acetonitrile and 10 mM aqueous triethylammonium acetate (30% to 50% CH₃CN over 20 minutes at 50 ml/min flow). Fractions from the preparative HPLC runs containing pure (6) were pooled, evaporated to remove CH₃CN and lyophilized to give 360 mg of pure (6) (the RpRp diastereoisomer) as the bis-triethylammonium salt.

To 270 mg (0.24 mmol) of (6) was added 5.0 ml of neat trimethylamine trihydrofluoride. The mixture was stirred at room temperature for approximately 40 h. After confirming completion of reaction by analytical HPLC, the sample was neutralized by dropwise addition into 45 ml of chilled, stirred 1M triethylammonium bicarbonate. The neutralized solution was desalted on a Waters C-18 Sep-Pak and the product eluted with CH₃CN/10 mM aqueous triethylammonium acetate (5:1).The CH₃CN was evaporated under reduced pressure and the remaining aqueous solution was frozen and lyophilized. Multiple rounds of lyophilization from water gave 122 mg (57%) of (T1-2) as the bis-triethylammonium salt. ¹H NMR (500 MHz, 45° C., (CD₃)₂SO-15 μL D₂O) δ 8.58 (s, 1H), 8.41 (s, 1H), 8.18 (s, 1H), 8.15 (s, 1H), 6.12 (d, J=8.0, 1H), 5.92 (d, J=7.0, 1H), 5.30 (td, J=8.5, 4.0, 1H), 5.24-5.21 (m, 1H), 5.03 (dd, J=7.5, 4.5, 1H), 4.39 (d, J=4, 1H), 4.23 (dd, J=10.5, 4.0, 1H), 4.18 (s, 1H), 4.14-4.08 (m, 2H), 3.85-3.83 (m, 1H), 3.73 (d, J=12.0, 1H), 3.06 (q, J=7.5, 12H), 1.15 (t, J=7.5, 1H); ³¹P NMR (200 MHz, 45° C., (CD₃)ISO-15pL D₂O) δ 58.81, 52.54; HRMS (FT-ICR) l/z calcd for C20H24O10N10P2S2 (M−H) 689.0521, found 689.0514.

### Example Synthesis of Compounds of Formula (A)

Synthesis of (2R,3R,3aS,5R,7aR,9S,10R,10aS,12R,14aR)-2,9-bis(6-amino-9H-purin-9-yl)-5,12-dimercaptotetrahydro-2H,7H,9H,14H-3,14a: 10,7a-bis(epoxymethano)difuro[3,2-d:3′,2′-j][1,3,7,9]tetraoxa[2,8]diphosphacyclododecine 5,12-dioxide (T2-45) and (2R,3R,3aS,5R,7aR,9S,10R,10aS,12S,14aR)-2,9-bis(6-amino-9H-purin-9-yl)-5,12-dimercaptotetrahydro-2H,7H,9H,14H-3,14a: 10,7a-bis(epoxymethano)difuro[3,2-d:3′,2′-j][1,3,7,9]tetraoxa[2,8]diphosphacyclododecine 5,12-dioxide (T2-44), were prepared according to the following Scheme:

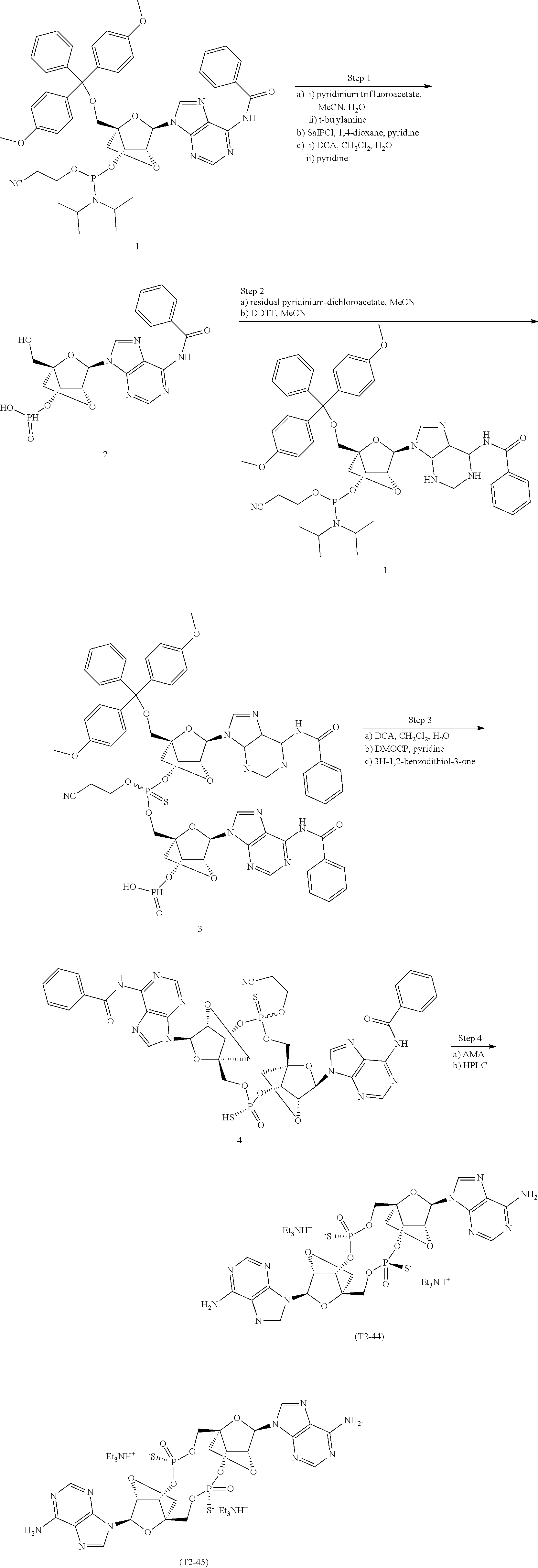

Step 1:

Preparation of (1S,3R,4R,7S)-3-(6-benzamido-9H-purin-9-yl)-1-(hydroxymethyl)-2,5-dioxabicyclo[2.2.1]heptan-7-yl hydrogen phosphonate (2): To a solution of (1R,3R,4R,7S)-3-(6-benzamido-9H-purin-9-yl)-1-((bis(4-methoxyphenyl)(phenyl)methoxy)methyl)-2,5-dioxabicyclo[2.2.1]heptan-7-yl (2-cyanoethyl) diisopropylphosphoramidite (1, 1.0 g, 1.2 mmol, Exiqon, Woburn, Mass.) in MeCN (10 mL) and H₂O (0.05 mL) was added pyridinium trifluoroacetate (270 g, 1.5 mmol). After 25 min, to the stirring reaction mixture at room temperature was added tert-butyl amine (5.0 mL). After 15 min, the reaction solution was concentrated in vacuo and water was removed as an azeotrope after concentration with MeCN (3×15 mL) to obtain a white foam. To a solution of the white foam in 1,4-dioxane (13 mL) was added a solution of SalPCI (226 mg, 1.0 mmol), in 1,4-dioxane (5 mL). After 7 min, to the cloudy white mixture was added pyridine (3 mL). After 1 h, to the cloudy reaction mixture was introduced water (2 mL). After 5 min, the mixture was poured into a 1N NaHCO₃ solution (100 mL). The solution was extracted with EtOAc (3×100 mL) and the organic layer was condensed to dryness in vacuo. The residue was dissolved in CH₂Cl₂ (10 mL) to give a white mixture. To this solution was added water (150 μL) and 9% (v/v) solution of DCA in CH₂Cl₂ (10 mL). After 10 min of stirring at room temperature, to the orange solution was charged pyridine (1.5 mL). The resulting clear solution was concentrated in vacuo and water was removed as an azeotrope after concentration with MeCN (3×20 mL). On the last evaporation, the resulting cloudy slurry of compound 2 was left in MeCN (20 mL).

Step 2:

Preparation of (1R,3R,4R,7S)-3-(6-benzamido-9H-purin-9-yl)-1-((((((1R,3R,4R,7S)-3-(6-benzamido-9H-purin-9-yl)-1-((bis(4-methoxyphenyl)(phenyl)methoxy)methyl)-2,5-dioxabicyclo[2.2.1]heptan-7-yl)oxy) (2-cyanoethoxy) phosphorothioyl)oxy)methyl)-2,5-dioxabicyclo[2.2.1]heptan-7-yl hydrogen phosphonate (3): A solution of compound 1 (1.0 g, 1.2 mmol, Exiqon) in MeCN (10 mL) was dried through concentration in vacuo. This process was repeated two more times to remove water as an azeotrope. On the last azeotrope, to the solution of compound 1 in MeCN (10 mL) was introduced ten 3 Å molecular sieves and the solution was stored under an atmosphere of nitrogen. To a stirred mixture of compound 2 with residual pyridinium dichloroacetate in MeCN (20 mL) was added the solution of compound 1 in MeCN (10 mL). After 40 min, to the stirred mixture was added DDTT (263 mg, 1.3 mmol). After 70 min, the yellow solution was concentrated in vacuo to give compound 3 as a yellow paste.

Step 3:

Preparation of N,N′-(((2S,3R,3aS,7aR,9R,10R,10aS,12R,14aR)-5-(2-cyanoethoxy)-12-mercapto-12-oxido-5-sulfidotetrahydro-2H,7H,9H, 14H-3,14a:10,7a-bis(epoxymethano)difuro[3,2-d:3′,2′-j][1,3,7,9]tetraoxa[2,8]diphosphacyclododecine-2,9-diyl)bis(9H-purine-9,6-diyl))dibenzamide (4): To a solution of compound 3 in CH₂Cl₂ (30 mL) were added water (180 μL) and 8.5% (v/v) solution of DCA in CH₂Cl₂ (20 mL). After stirring for 15 min at room temperature, to the red-orange solution was introduced pyridine (10 mL). The resulting yellow solution was concentrated in vacuo until approximately 10 mL of the yellow mixture remained. To the yellow mixture was introduced pyridine (30 mL) and the mixture was concentrated in vacuo until approximately 10 mL of the yellow mixture remained. To the yellow mixture was added pyridine (30 mL) and the mixture was concentrated in vacuo until approximately 10 mL of the yellow mixture remained. To the stirred yellow mixture in pyridine (50 mL) was added DMOCP (631 mg, 3.4 mmol). After 15 min, to the brownish yellow solution was added water (750 μL), followed immediately by the introduction of 3H-1,2-benzodithiol-3-one (304 mg, 1.8 mmol). After 30 min, the brownish yellow solution was poured into a 1N aqueous NaHCO₃ solution (250 mL). After 15 min, the biphasic mixture was extracted with EtOAc (200 mL). After separation, the aqueous layer was back extracted with EtOAc (2×150 mL). The organic extracts were combined and concentrated in vacuo. To the concentrated yellow oil was added toluene (20 mL) and the mixture was evaporated in vacuo to remove residual pyridine. This procedure was repeated again with toluene (30 mL). The resulting oil was purified by silica gel chromatography (0% to 50% MeOH in CH₂Cl₂) to provide a mixture of compound 4 (604 mg, 52% yield) as beige solid.

Step 4:

Preparation of (T2-45) and (T2-44): To a stirred solution of compound 4 (472 mg, 0.5 mmol) in EtOH (5.0 mL) was added AMA (ammonium hydroxide/40% methylamine solution in water )(6.5 mL) and the yellow solution was heated at 50° C. After 2 h, the yellow solution was allowed to cool and concentrated in vacuo. The yellow residue in 10 mM TEAA (3 mL) was purified by reverse phase silica gel chromatography (0% to 25% MeCN in 10 mM aqueous TEAA) to obtain compound (T2-45) (92 mg, 27% yield) as a white triethylammonium salt after lyophilization. LCMS-ESI: 712.95 [M−H]− (calculated for C₂₂H₂₄N₁₀O₁₀P₂S₂: 714.56); Rₜ: 1.06 min by UPLC (20 mM NH₄OAc, 2% to 80% MeCN). ¹H NMR (400 MHz, 45° C., D₂O) δ 8.45 (d, J=4.4 Hz, 2H), 8.30 (d, J=5.6 Hz, 2H), 6.36 (d, J=4.4 Hz, 2H), 5.12 (s, 4H), 4.63 (d, J=12.4 Hz, 2H), 4.34-4.24 (m, 6H), 3.33 (q, J=7.2 Hz, 12H), 2.09 (m, 1H), 1.40 (t, J=5.2 Hz, 18H). ³¹P NMR (45° C., D₂O) δ 54.57.

The Rp,Sp isomer was also isolated after purification in the reverse phase chromatography step, to provide compound (T2-44) (35 mg, 10% yield) as the triethylammonium salt after lyophilization. LCMS-ESI: 712.95 [M−H]− (calculated for C₂₂H₂₄N₁₀O₁₀P₂S₂: 714.56); Rₜ: 1.01 min by UPLC (20 mM NH₄OAc, 2% to 80% MeCN). ¹H NMR (400 MHz, 45° C., D₂O) δ 8.58 (s, 1H), 8.46 (s, 1H), 8.31 (s, 1H), 8.27 (s, 1H), 6.38 (s, 2H), 5.32 (s, 1H), 5.11 (s, 1H), 5.07 (d, J=10.4 Hz, 2H), 4.62 (d, J=11.2 Hz, 1H), 4.53 (d, J=11.2 Hz, 1H), 4.41-4.31 (m, 4H), 4.24 (t, J=16.4 Hz, 1H), 3.33 (q, J=7.2 Hz, 10H), 1.41 (t, J=7.2 Hz, 15H). ³¹P NMR (45° C., D₂O) δ 55.33, 54.48.

### Example Synthesis of Compounds of Formula (B)

Certain compounds of Formula (B) were made enzymatically. Specifically compound T1-25 was prepared enzymatically according to the following synthetic scheme:

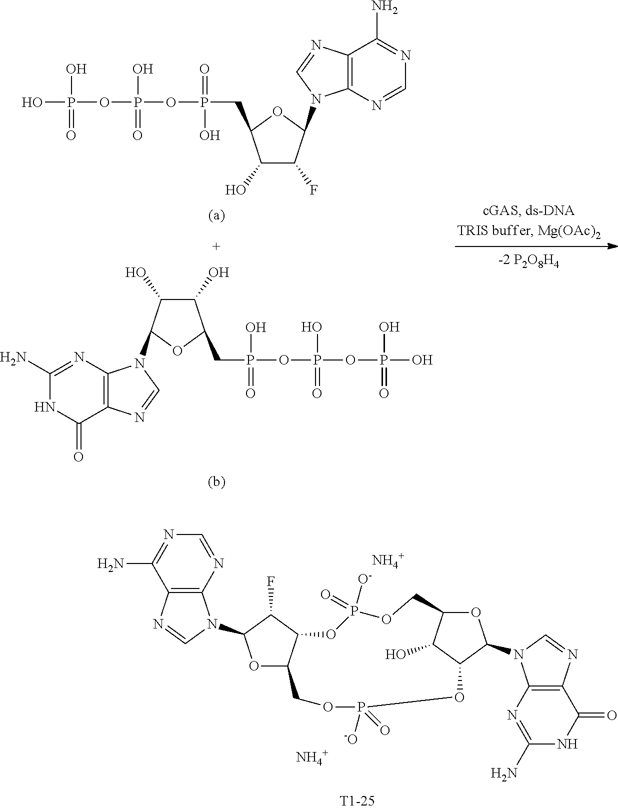

The reaction was carried out in duplicate in parallel: to 100 mM aqueous (((2S,3R,4R,5R)-5-(6-amino-9H-purin-9-yl)-4-fluoro-3-hydroxytetrahydrofuran-2-yl)methyl) phosphonic diphosphoric anhydride (a) (250 μL, 0.025 mmol; N-1007, TriLink Biotechnologies, San Diego, Calif., USA), 100 mM aqueous (((2S,3S,4R,5R)-5-(2-amino-6-oxo-1,6-dihydro-9H-purin-9-yl)-3,4-dihydroxytetrahydrofuran-2-yl)methyl)phosphonic diphosphoric anhydride (b) (250 μL, 0.025 mmol, Sigma Cat. No 51120), Herring Sperm DNA solution (250 μL, 10 mg/mL aq.; #9605-5-D, Trevigen Inc., Gaithersburg, Md., USA) and human cGAS (1500 μL, 2.1 mg/mL, prepared as described in the next paragraph) was added reaction buffer (50 mM TRIS, 2.5 mM magnesium acetate, 10 mM KCl, pH adjusted to 8.2 with aq. NaOH 5 M; 25 mL). The reaction was incubated for 16 hours at 37° C. and 150 rpm on an orbital shaker. Completion of the reaction was confirmed through analysis of an aliquot (100 μL) of the reaction mixture, diluted with acetonitrile (100 μL), centrifuged and the desired compound formation determined by UV analysis. The reactions were mixed with acetonitrile (20 mL), incubated at room temperature on an orbital shaker for 10 minutes and after subsequent centrifugation (7000 g for 5 min) the supernatant was filtrated through a paper filter. The filtrate was mixed with acetic acid (100 μL) and directly loaded onto a 20×250 mm Inertsil Amide 5 μm column (flow rate 30 mL/min; solvent A: aqueous 10 mM ammonium acetate, 2 mM acetic acid, solvent B: acetonitrile; using an isocratic elution using 26% phase A/74% phase B, fraction size 50 mL). The fractions containing the desired compound (T1-25) were combined and the solvents were evaporated in vacuo to a final volume of about 10 mL. The concentrated compound (T1-25) solution from the first chromatography was re-purified by direct injection onto 1×50 cm Sephadex G10 HPLC column (flow rate 1.0 mL/min; mobile phase containing 0.25 mM ammonium hydroxide and 25% acetonitrile) with UV detection at 250 nm. All fractions containing the desired compound (T1-25) were combined and dried by lyophilisation to give 4.5 mg of compound (T1-25) as the bis-ammonium salt; ¹H NMR (600.1 MHz, D₂O) δ 8.35 (br s, 1H), 8.06 (br s, 1H), 7.77 (s, 1H), 6.31 (d, J=12.8 Hz, 1H), 5.86 (s, 1H), 5.62 (s, 1H), 5.35 (d, J=50.8 Hz, 1H), 4.97 (d, J=19.0 Hz, 1H), 4.46 (s, 1H), 4.42 (s, 1H), 4.33 (s, 1H), 4.24 (s, 1H), 4.21 (s, 2H), 3.97 (s, 1H); MS m/z 677.2 [M+H]+.

The cGAS used in this example and the following example were prepared by cloning and expression of human and mouse cGAS. The coding region of human or mouse cGAS comprising amino acid 155-522 (human) and amino acid 147-507 (mouse) was cloned into a pET based expression vector. The resulting expression construct contained an N-terminal 6×-His-tag (SEQ ID NO: 930) followed by a ZZ-tag and an engineered HRV3C protease cleavage side allowing generation of human cGAS 155-522 and mouse cGas 147-507 with an N-terminal extension of a Gly-Pro. Both plasmids were transformed in the E. coli strain * BL21 (DE3) phage resistant cells (C2527H, New England BioLabs, Ipswich, Mass.) for bacterial expression. The phage resistant E. coli cells BL21(DE3) harboring the cGas expression plasmids were expressed at a 1.5 L scale in Infors bioreactors. Precultures were grown in LB medium. 1.5 L auto-induction media (Studier, Protein Expr Purif. 2005 May; 41(1):207-34) containing Kanamycin (50 [symbol] g/mL) were inoculated with 100 mL preculture and cultivated to an OD of approximately 10 under the following conditions: temperature 37° C.; stirrer (cascade regulation via pO2) 500; pH 7.0; pO2 (cascade regulation on) 5%; flow 2.5 L/min; and gas mix (cascade regulation via pO2) 0. The temperature was then reduced to 18° C. and expression was run over night. Cells were harvested by centrifugation and lysed by using an Avestin EmulsiFlex French press. Purification was done according the published protocol by Kato et al. (PLoS One, 2013, 8(10) e76983) using Ni-affinity chromatography, a heparin purification step to remove DNA and a final size exclusion chromatography. cGAS eluted as a homogenous fraction and was concentrated to at least 5 mg/mL.

(SEQ ID NO: 940)
Human cGAS: GPDAAPGASK LRAVLEKLKL SRDDISTAAG MVKGVVDHLL LRLKCDSAFR
GVGLLNTGSY YEHVKISAPN EFDVMFKLEV PRIQLEEYSN TRAYYFVKFK RNPKENPLSQ
FLEGEILSAS KMLSKFRKII KEEINDIKDT DVIMKRKRGG SPAVTLLISE KISVDITLAL
ESKSSWPAST QEGLRIQNWL SAKVRKQLRL KPFYLVPKHA KEGNGFQEET WRLSF-SHIEK
EILNNHGKSK TCCENKEEKC CRKDCLKLMK YLLEQLKERF KDKKHLDKFS SYHVKTAFFH
VCTQNPQDSQ WDRKDLGLCF DNCVTYFLQC LRTEKLENYF IPEFNLFSSN LIDKRSKEFL
TKQIEYERNN EFPVFDEF.

### Example Synthesis of Compounds of Formula (B)

Certain compounds of Formula (B) were made enzymatically. Specifically compound T1-28 was prepared enzymatically according to the following synthetic scheme:

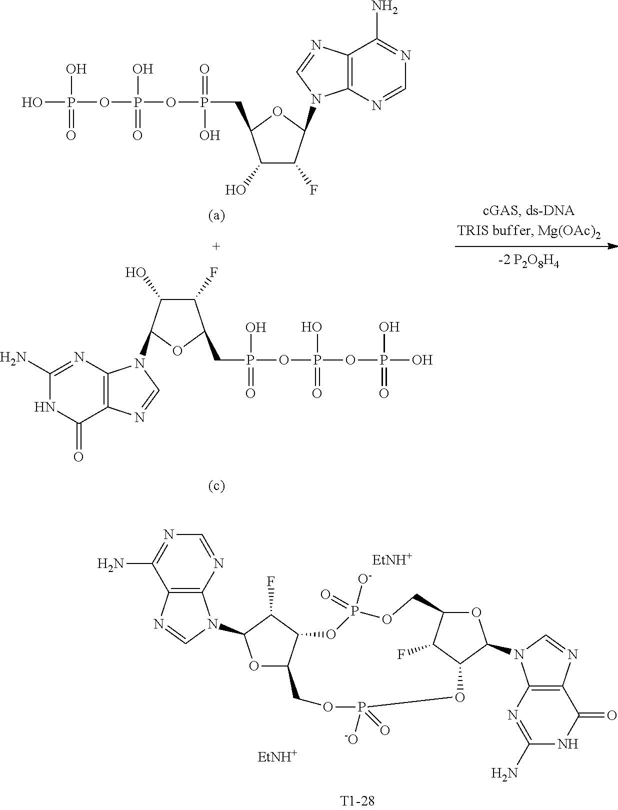

The reaction was performed four times in parallel, each on a 26 mL scale: to 100 mM aqueous (((2S,3R,4R,5R)-5-(6-amino-9H-purin-9-yl)-4-fluoro-3-hydroxytetrahydrofuran-2-yl)methyl)phosphonic diphosphoric anhydride (a) (250 μL, 0.025 mmol), 100 mM aqueous (((2S,3S,4S,5R)-5-(2-amino-6-oxo-1,6-dihydro-9H-purin-9-yl)-3-fluoro-4-hydroxytetrahydrofuran-2-yl)methyl)phosphonic diphosphoric anhydride (c) (250 μL, 0.025 mmol; N-3002, TriLink Biotechnologies), Herring Sperm DNA solution (800 μL, 10 mg/mL aq.; #9605-5-D, Trevigen Inc.) and mouse cGAS preparation (250 μL, 6.5 mg/mL, prepared as described for human cGAS above) was added reaction buffer (50 mM TRIS, 2.5 mM magnesium acetate, pH adjusted to 8.2 with aq. NaOH 5 M; 25 mL). The reaction was incubated for 16 hours at 37° C. and 150 rpm on an orbital shaker. The reactions were mixed with acetonitrile (20 mL) and incubated at room temperature on an orbital shaker for 10 min. After subsequent centrifugation (7000 g for 5 min) the supernatant of all four reactions was combined and filtrated through a paper filter. The filtrate was evaporated in vacuo to a residual volume of approximately 20 mL and mixed with 0.5 mL acetic acid (0.5 mL) and 1.0M aqueous triethylammonium acetate (5 mL). The crude material was directly injected onto the Chromolith RP18e 2.1×10 cm column. Chromatography (flowrate 80 mL/min; isocratic mobile 10 mM triethylammonium acetate and 1 vol % acetonitrile) yielded the desired compound (T1-28) fractions which were combined, mixed with aqueous 25% ammonia solution (20 μL) and dried by lyophilisation. The compound (T1-28) was obtained as bis-triethylammonium salt; 39.8 mg; ¹H NMR (600.1 MHz, D₂O) δ 8.16 (s, 1H), 8.13 (s, 1H), 7.73 (s, 1H), 6.33 (d, J=13.9 Hz, 1H), 5.91 (d, J=8.6 Hz, 1H), 5.61 (m, 1H), 5.40 (dd, J=51.5, 2.6 Hz, 1H), 5.30 (dd, J=53.3, 3.2 Hz, 1H), 4.98 (m, 1H), 4.56 (d, J=25.8 Hz, 1H), 4.44 (d, J=9.0 Hz, 1H), 4.39 (d, J=11.8 Hz, 1H), 4.20 (m, 1H), 4.08 (d, J=12.4 Hz, 1H), 4.04 (d, J=11.8 Hz, 1H), 3.06 (q, J=7.3 Hz, 12H), 1.13 (t, J=7.3 Hz, 18H); 31P NMR (376.4 MHz, D₂O) δ −1.68, −2.77; 19F NMR (376.4 MHz, D₂O) δ −199.72, −203.23; MS 677.2 [M−1]−.

(SEQ ID NO: 941)
Mouse cGAS: GPDKLKKVLD KLRLKRKDIS EAAETVNKVV ERLLRRMQKR ESEFKGVEQL
NTGSYYEHVK ISAPNEFDVM FKLEVPRIEL QEYYETGAFY LVKFKRIPRG NPL-SHFLEGE
VLSATKMLSK FRKIIKEEVK EIKDIDVSVE KEKPGSPAVT LLIRNPEEIS VDIILALESK
GSWPISTKEG LPIQGWLGTK VRTNLRREPF YLVPKNAKDG NSFQGETWRL SF-SHTEKYIL
NNHGIEKTCC ESSGAKCCRK ECLKLMKYLL EQLKKEFQEL DAFCSYHVKT AIFHMWTQDP
QDSQWDPRNL SSCFDKLLAF FLECLRTEKL DHYFIPKFNL FSQELIDRKS KEFLSKKIEY
ERNNGFPIFD KL.

### Example Synthesis of Compounds of Formula (D)

Specifically, (1S,3R,6R,8R,9S,11R,14R,16R,17R,18R)-8,16-bis(6-amino-9H-purin-9-yl)-17,18-difluoro-2,4,7,10,12,15-hexaoxa-3,11-diphosphatricyclo[12.2.1.16,9]octadecane-3,11-bis(thiolate) 3,11-dioxide (8) (which corresponds to compound (T2-46)) was synthesized according to the scheme below:

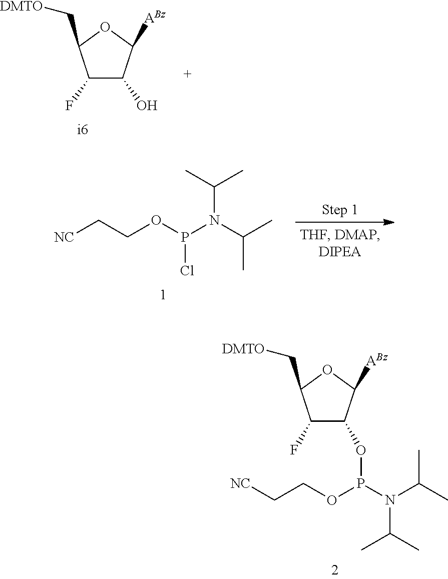

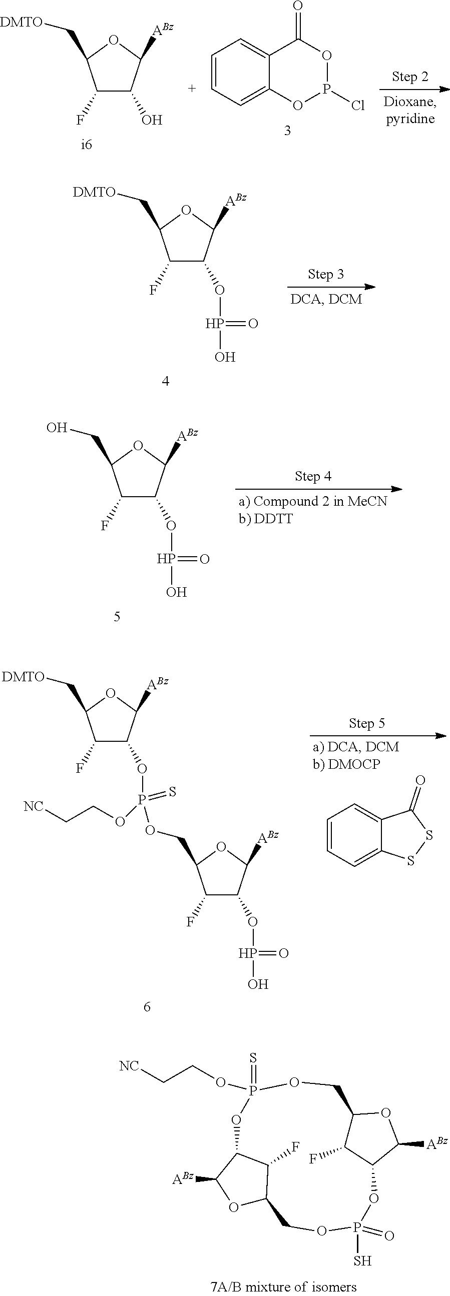

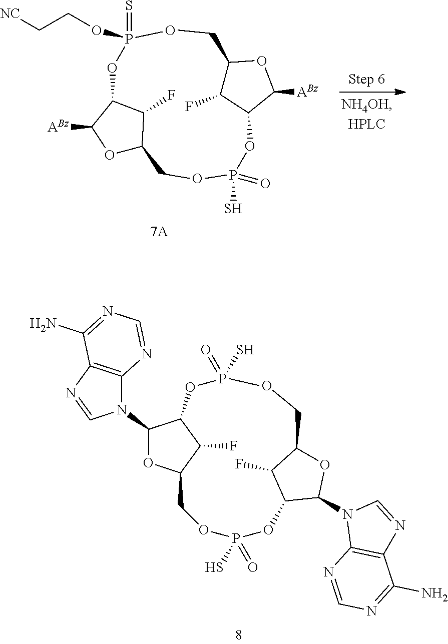

Step 1:

Preparation of (2R,3S,4R,5R)-2-(6-benzamido-9H-purin-9-yl)-5-((bis(4-methoxyphenyl) (phenyl)methoxy)methyl)-4-fluorotetrahydrofuran-3-yl (2-cyanoethyl) diisopropylphosphoramidite (2): To a solution of Compound i6 (1, 1 g, 1.5 mmol, 1 eq) (dried via co-evaporation in vacuo with anhydrous MeCN (3×3 mL)) in anhydrous THF (6 mL) was added DMAP (18 mg, 0.15 mmol, 0.1 eq) and DIPEA (0.98 mL, 5.9 mmol, 4 eq). 2-cyanoethyl N,N-diisopropyl chlorophosphoramidite (360 μL, 1.6 mmol, 1.1 eq, ChemGenes) was added and the reaction was stirred overnight. The mixture was diluted with 100 mL of EtOAc (prewashed with 5% NaHCO₃) and washed with brine (5×50 mL). The EtOAc layer dried over Na₂SO₄, filtered and concentrated in vacuo. Flash chromatography (40 g silica gel, isocratic gradient—50:44:4 DCM:Hexanes:TEA) gave 1.08 g of the compound 2.

Step 2:

Preparation of (2R,3S,4R,5R)-2-(6-benzamido-9H-purin-9-yl)-5-((bis(4-methoxyphenyl)(phenyl)methoxy)methyl)-4-fluorotetrahydrofuran-3-yl hydrogen phosphonate (4): To a solution of Compound i6 (1.5 g, 2.7 mmol, 1 eq) in anhydrous dioxane (17 mL) was added anhydrous pyridine (4.7 mL, 69 mmol, 26 eq) followed by a solution of 2-chloro-1,3,2-benzodioxaphosphorin-4-one (3, 540 mg, 3.2 mmol, 1.2 eq, Sigma Aldrich) in 1,4-dioxane (8.3 mL). The reaction mixture was stirred for 1 h then diluted with 10 mL water and NaHCO₃ (3.72 g in 100 mL of water). The suspension was extracted with EtOAc (3×100 mL), the organic layers were combined, dried with Na₂SO₄, filtered and concentrated. Chromatography (80 g of SiO₂, 0-50% MeOH (with 0.5% pyridine) and DCM) gave compound 4.

Step 3:

Preparation of (2R,3S,4R,5R)-2-(6-benzamido-9H-purin-9-yl)-4-fluoro-5-(hydroxymethyl)tetrahydrofuran-3-yl hydrogen phosphonate (5): To a solution of compound 4 (0.78 g, 1.1 mmol, 1 eq) in DCM (13 mL) was added water (190 μL, 11 mmol, 10 eq) and a solution of DCA (760 μL 9.2 mmol, 8.7 eq) in DCM (13 mL). The mixture was stirred for 10 min and quenched with pyridine (1.5 mL, 18 mmol, 17 eq). The mixture was concentrated in vacuo and co-evaporated with anhydrous MeCN (3×10 mL) to provide compound 5 in 4 mL of MeCN.

Step 4:

Preparation of (2R,3S,4R,5R)-2-(6-benzamido-9H-purin-9-yl)-5-((((((2R,3S,4R,5R)-2-(6-benzamido-9H-purin-9-yl)-5-((bis(4-methoxyphenyl)(phenyl)methoxy)methyl)-4-fluorotetrahydrofuran-3-yl)oxy)(2-cyanoethoxy)phosphorothioyl)oxy)methyl)-4-fluorotetrahydrofuran-3-yl hydrogen phosphonate (6): Compound 2 (1.1 g, 1.2 mmol, 1.1 eq) was dried via co-evaporation in vacuo with anhydrous MeCN (3×10 mL leaving 8 mL). This solution was added to the solution of compound 5 from Step 3 and stirred for 5 min. DDTT (240 mg, 1.2 mmol, 1.1 eq) was added and the mixture was stirred for 30 min then concentrated in vacuo to provide compound 6.

Step 5:

Preparation of N,N′-(((1 S,3R,6R,8R,9S,11R, 14R,16R,17R,18R)-3-(2-cyanoethoxy)-17,18-difluoro-11-mercapto-11-oxido-3-sulfido-2,4,7,10,12,15-hexaoxa-3,11-diphosphatricyclo[12.2.1.1⁶⁹]octadecane-8,16-diyl)bis(9H-purine-9,6-diyl))dibenzamide (7A): To a solution of compound 6 in DCM (25 mL) was added water (190 μL, 11 mmol, 10 eq) and a solution of DCA (1.5 mL, 18 mmol, 17 eq) in DCM (25 mL). The mixture was stirred for 10 min, then quenched with pyridine (11 mL, 130 mmol, 120 eq), then concentrated in vacuo to approximately 13 mL. An additional 30 mL of anhydrous pyridine was added. The solution was treated with DMOCP (580 mg, 3.2 mmol, 3 eq) and stirred for 3 min, after which water (570 μL, 32 mmol, 30 eq) was added followed immediately by 3H-1,2-benzodithiol-3-one (260 mg, 1.6 mmol, 1.5 eq). After 5 min the solution was poured into saturated NaHCO₃ (100 mL) and extracted with EtOAc (2×100 mL). The organic layers were combined and concentrated to give ˜2.5 g of crude mixture of isomers 7A/B. Chromatography (80 g SiO₂, MeOH:DCM 0-15% over 54 min) gave 128 mg of compound 7A.

Step 6:

Preparation of (1S,3R,6R,8R,9S,11R,14R,16R,17R,18R)-8,16-bis(6-amino-9H-purin-9-yl)-17,18-difluoro-3,11-dimercapto-2,4,7,10,12,15-hexaoxa-3,11-diphosphatricyclo[12.2.1.1^{6,9}]octadecane 3,11-dioxide (8) (which corresponds to compound (T2-46)): To a solution of 7A (70 mg) in MeOH (1.5 mL) was added NH₄OH (1.5 mL). The reaction mixture was heated to 50° C. for 2.5 h then cooled, sparged with N₂ and concentrated in vacuo. Purification (RP MPLC—5.5 g C18—0-20% MeCN/TEAA (10 mM) over 90 column volumes) to give after lyophilization 10 mg of Compound 8. LCMS-ESI: 693.70 [M−H]− (calculated for C₂₀H₂₂F₂N₁₀O₈P₂S₂: 694.05); Rₜ: 8.174 min by LCMS conditions (20 mM NH₄OAc, 2% to 50%). ¹H NMR. (400 MHz, 45° C., D₂O) δ 8.08 (s, 1H), 7.99 (s, 1H), 6.17 (d, J=8.4, 1H), 5.84 (dd, J=52.4, 3.6 1H), 5.19-5.11 (m, 1H), 4.77 (m, 1H), 4.46-4.2 (m, 1H), 4.10-4.09 (m, 1H), 3.09 (q, J=7.2, 6H), 1.17 (t, J=7.6 Hz, 9H).

Intermediate i6 (used above) was prepared according to the following scheme

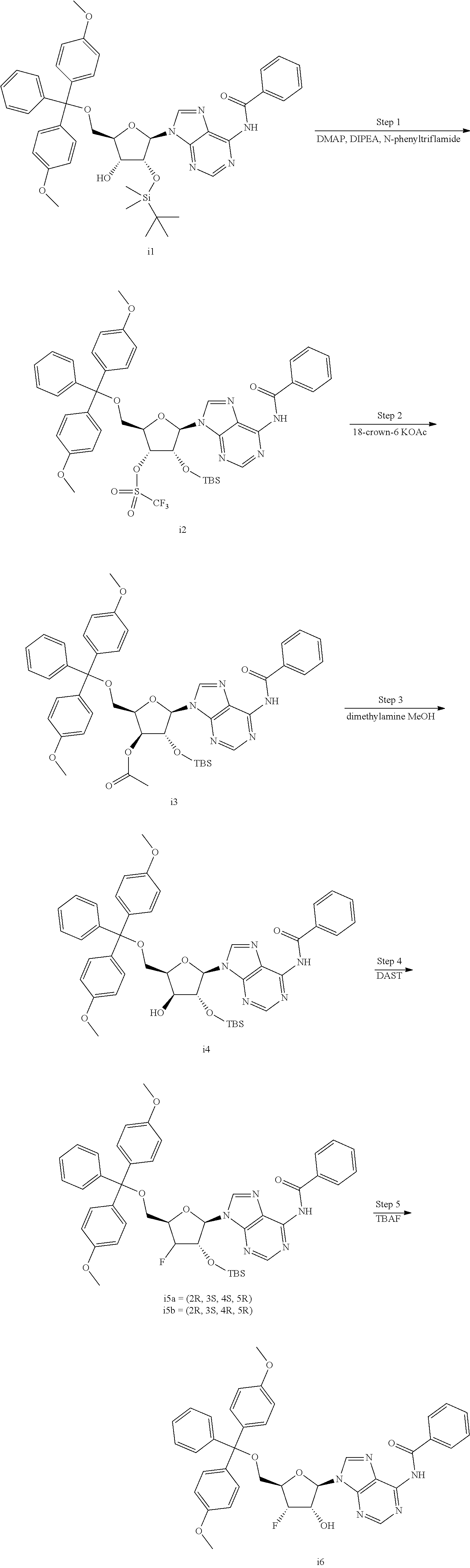

Step 1:

Preparation of (2R,3R,4R,5R)-5-(6-benzamido-9H-purin-9-yl)-2-((bis(4-methoxyphenyl) (phenyl)methoxy)methyl)-4-((tert-butyldimethylsilyl)oxy)tetrahydrofuran-3-yl trifluoromethane-sulfonate (i2): A mixture of N-(9-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-9H-purin-6-yl)benzamide (i1, 5.6 g, 7.11 mmol, ChemGenes) and DMAP (0.174 g, 1.42 mmol) was suspended in anhydrous THF (35 mL), addition of DIPEA (6.21 mL, 35.5 mmol) created a solution to which N-phenyltriflamide (5.08 g, 14.21 mmol), was added. The mixture was stirred for 3.5 h at rt, at which point it was poured into 5% brine (100 mL) and extracted with EtOAc (2×100 mL). The combined organic phases were dried (Na₂SO₄) the drying agent filtered-off and concentrated on silica gel (10 g) in vacuo. The crude material was purified by chromatography on silica gel (gradient elution 25-100% EtOAc/heptane) to give the desired compound i2 as a tan solid; 5.53 g; ¹H NMR (400 MHz, CDCl₃) δ 9.05 (s, 1H), 8.68 (s, 1H), 8.18 (s, 1H), 8.06 (d, J=7.5 Hz, 2H), 7.66 (t, J=7.4 Hz, 1H), 7.61-7.48 (m, 4H), 7.48-7.25 (m, 7H), 6.88 (d, J=8.8 Hz, 4H), 6.04 (d, J=7.6 Hz, 1H), 5.50 (dd, J=7.5, 4.7 Hz, 1H), 5.32 (d, J=4.5 Hz, 1H), 4.50 (t, J=4.1 Hz, 1H), 3.82 (s, 6H), 3.77 (dt, J=10.8, 5.2 Hz, 1H), 3.41 (dd, J=10.8, 3.7 Hz, 1H), 0.77 (s, 9H), −0.01 (s, 3H), −0.46 (s, 3H); LCMS (Method A) Rt=1.65 min; m/z 920.5 [M+H]⁺.

Step 2:

Preparation of (2R,3S,4R,5R)-5-(6-benzamido-9H-purin-9-yl)-2-((bis(4-methoxyphenyl) (phenyl)methoxy)methyl)-4-((tert-butyldimethylsilyl)oxy)tetrahydrofuran-3-yl acetate (i3): A mixture of compound i2 (5.5 g, 5.98 mmol), KOAc (2.93 g, 29.9 mmol), and 18-crown-6 (1,4,7,10,13,16-hexaoxacyclooctadecane, 0.79 g, 2.99 mmol) in toluene (40 mL) was heated at 110° C. for 4 h. The reaction mixture was then cooled to rt and silica gel (10 g) added and the solvent was removed in vacuo. The crude material was purified by chromatography on silica gel (gradient elution 25-100% EtOAc/heptane) to give the desired compound i3 as a tan solid: 3.3 g; ¹H NMR (400 MHz, CDCl₃) δ 8.70 (s, 1H), 8.58 (s, 1H), 7.93 (s, 1H), 7.84 (d, J=7.5 Hz, 2H), 7.44 (t, J=7.4 Hz, 1H), 7.35 (t, J=7.6 Hz, 2H), 7.28 (d, J=7.2 Hz, 2H), 7.21-7.02 (m, 7H), 6.67 (dd, J=8.9, 2.1 Hz, 4H), 5.98 (s, 1H), 4.97 (dd, J=3.6, 1.4 Hz, 1H), 4.61-4.52 (m, 1H), 4.35 (s, 1H), 3.62 (s, 6H), 3.41 (dd, J=9.8, 6.2 Hz, 1H), 3.18 (dd, J=9.8, 5.6 Hz, 1H), 1.53 (s, 3H), 0.77 (s, 9H), 0.03 (s, 3H), 0.0 (s, 3H). LCMS (Method A) Rₜ 1.68 min; m/z 830.2 [M+H]⁺.

Step 3:

Preparation of N-(9-((2R,3R,4S,5R)-5-((bis(4-methoxyphenyl)(phenyl)methoxy)methyl)-3-((tert-butyldimethylsilyl)oxy)-4-hydroxytetrahydrofuran-2-yl)-9H-purin-6-yl)benzamide (i4): Compound i3 (6.78 g, 8.17 mmol) was dissolved in MeOH (120 mL) and a 2.0 M dimethylamine solution in MeOH (20.4 mL, 40.8 mmol) was added. The reaction mixture was stirred for 17 h at rt. Silica gel (12 g) was added and the solvent was removed in vacuo. The crude material was purified by chromatography on silica gel (gradient elution 25-75% EtOAc/heptane) to give the desired compound i4 as a tan solid: 3.9 g; ¹H NMR (400 MHz, CDCl₃) δ 8.94 (s, 1H), 8.65 (s, 1H), 8.16 (s, 1H), 7.97-7.90 (m, 2H), 7.58-7.38 (m, 3H), 7.38-7.32 (m, 2H), 7.32-7.00 (m, 7H), 6.80-6.65 (m, 4H), 5.83 (d, J=1.2 Hz, 1H), 5.38 (d, J=8.0 Hz, 1H), 4.42 (s, 1H), 4.29 (t, J=4.6 Hz, 1H), 4.02-3.95 (m, 1H), 3.75-3.61 (m, 6H), 3.53 (d, J=5.0 Hz, 2H), 0.81 (s, 9H), 0.0 (s, 6H). LCMS (Method A) Rₜ 1.57 min; m/z 788.2 [M+H]⁺.

Step 4:

Preparation of N-(9-((2R,3S,4S,5R)-5-((bis(4-methoxyphenyl)(phenyl)methoxy)methyl)-3-((tert-butyldimethylsilyl)oxy)-4-fluorotetrahydrofuran-2-yl)-9H-purin-6-yl)benzamide (i5a) and N-(9-((2R,3S,4R,5R)-5-((bis(4-methoxyphenyl)(phenyl)methoxy) methyl)-3-((tert-butyldimethylsilyl)oxy)-4-fluorotetrahydrofuran-2-yl)-9H-purin-6-yl)benzamide (i5b): Compound i4 (750 mg, 0.952 mmol) was dissolved in anhydrous DCM (7 mL) under an inert nitrogen atmosphere and the solution was cooled to 0° C. A 1.0 M solution of DAST (1.90 mL, 1.90 mmol) was added and the reaction subsequently stirred at −5° C. for 17 h using a cryo-cool to control the reaction temperature. The vessel was warmed to 0° C. and saturated NaHCO₃ (2 mL) was added. After 30 min of stirring the mixture was diluted with 5% brine (20 mL) and extracted with EtOAc (2×20 mL). The combined organics were dried (Na₂SO₄) with the drying agent filtered off, silica gel (2 g) added to the filtrate and the solvent removed in vacuo. The crude material was purified by chromatography on silica gel (gradient elution 10-75% EtOAc/heptane) to give a mixture of diastereoisomers i5a and i5b as a tan solid: 193 mg; Major (2R,3S,4S,5R) diastereoisomer LCMS (Method A) Rₜ 1.53 min; m/z 790.4 (M+H)⁺; Minor (2R,3S,4R,5R) diastereoisomer Rₜ 1.58 min; m/z 790.4 [M+H]⁺.

Step 5:

Preparation of N-(9-((2R,3S,4S,5R)-5-((bis(4-methoxyphenyl)(phenyl)methoxy)methyl)-4-fluoro-3-hydroxytetrahydrofuran-2-yl)-9H-purin-6-yl)benzamide (i6): The diastereomeric mixture of i5a and i5b (2.0 g, 2.53 mmol) was dissolved in anhydrous THF (100 mL) and cooled to −42° C. under an inert nitrogen atmosphere before 1.0 M TBAF (3.80 mL, 3.80 mmol) was added. The reaction was stirred for 2.5 h, then quenched with saturated NaHCO₃ (20 mL). The cold bath was removed, and the slurry was stirred for 10 min before the mixture was diluted with 5% brine (150 mL) and extracted with DCM (2×100 mL). The combined organic phases were dried (Na₂SO₄), with the drying agent filtered off, silica gel (4 g) added to the filtrate and the solvent removed in vacuo. The crude material was purified by chromatography on silica gel (gradient elution 25-100% EtOAc/heptane) to give the desired compound i6 as a white solid: 355 mg; ¹H NMR (400 MHz, CDCl₃) δ 9.16 (s, 1H), 8.64 (s, 1H), 8.23 (s, 1H), 7.99 (d, J=7.5 Hz, 2H), 7.59 (t, J=7.4 Hz, 1H), 7.48 (t, J=7.6 Hz, 2H), 7.41-7.31 (m, 3H), 7.31-7.11 (m, 7H), 6.79 (d, J=8.9 Hz, 4H), 6.16 (d, J=7.3 Hz, 1H), 5.77 (br s, 1H), 5.27-5.10 (m, 2H), 4.53 (dt, J=28.0 Hz, 3.4 Hz, 1H), 3.77 (s, 6H), 3.51 (dd, J=10.7, 3.7 Hz, 1H), 3.34 (dd, J=10.7, 3.3 Hz, 1H); ¹⁹F NMR (376.4 MHz, CDCl₃) δ −197.5; ¹³C NMR (101 MHz, CDCl₃) δ 164.66, 158.64, 158.62, 152.60, 151.43, 149.34, 144.22, 141.66, 135.29, 135.13, 133.40, 132.93, 129.96, 128.87, 127.99, 127.93, 127.86, 127.07, 122.65, 113.26, 93.85, 92.02, 87.56 (d, J=144 Hz), 83.56 (d, J=23 Hz), 77.30, 74.63 (d, J=16 Hz), 62.82 (d, J=11 Hz), 55.26; LCMS (Method A) Rₜ 0.89 min; m/z 676.3 [M+H]⁺.

Alternatively, Intermediate i6 was also prepared according to the following Scheme 1A′:

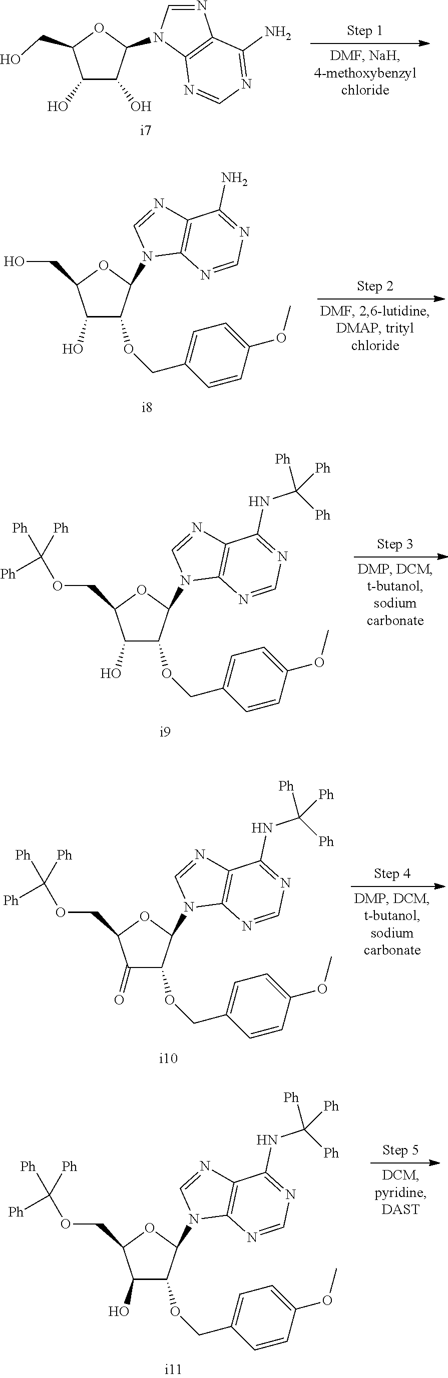 [SMILES: Nc1ncnc2c1ncn2[C@@H]1O[C@H](CO)[C@@H](O)[C@H]1O ; COc1ccc(CO[C@@H]2[C@H](O)[C@@H](CO)O[C@H]2n2cnc3c(N)ncnc32)cc1 ; COc1ccc(CO[C@@H]2[C@H](O)[C@@H](COC(c3ccccc3)(c3ccccc3)c3ccccc3)O[C@H]2n2cnc3c(NC(c4ccccc4)(c4ccccc4)c4ccccc4)ncnc32)cc1 ; COc1ccc(CO[C@@H]2C(=O)[C@@H](COC(c3ccccc3)(c3ccccc3)c3ccccc3)O[C@H]2n2cnc3c(NC(c4ccccc4)(c4ccccc4)c4ccccc4)ncnc32)cc1 ; COc1ccc(CO[C@@H]2[C@@H](O)[C@@H](COC(c3ccccc3)(c3ccccc3)c3ccccc3)O[C@H]2n2cnc3c(NC(c4ccccc4)(c4ccccc4)c4ccccc4)ncnc32)cc1]

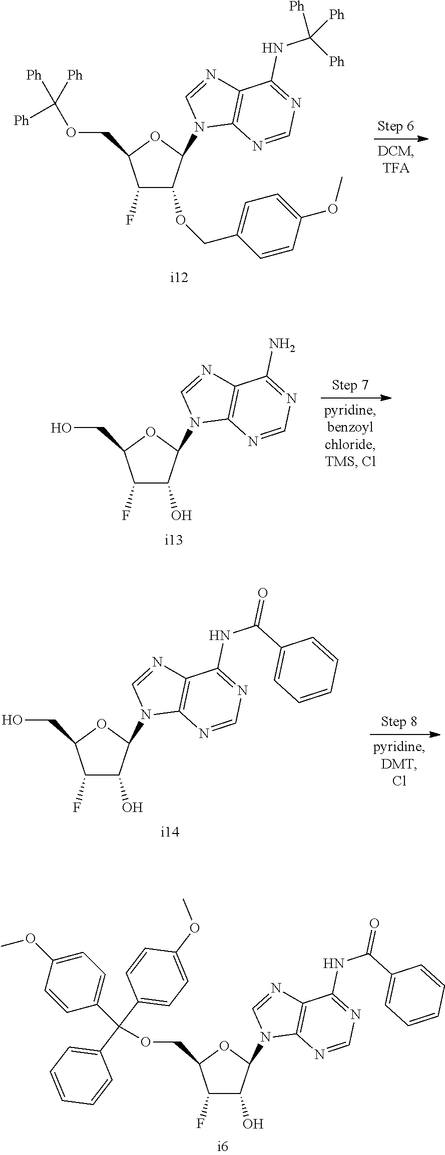

Step 1:

Preparation of (2R,3R,4R,5R)-5-(6-amino-9H-purin-9-yl)-2-(hydroxymethyl)-4-((4-methoxybenzyl) oxy)tetrahydrofuran-3-ol (i8): To a suspension of adenosine (i7, 100 g, 374 mmol) in DMF (2.64 L) at 4° C. under nitrogen was added 60% sodium hydride (19.46 g, 486 mmol) in one portion and the reaction mixture stirred under nitrogen for 60 min. 4-Methoxybenzyl chloride (60.9 ml, 449 mmol) was added dropwise over a 10 min period and the suspension stirred and warmed to rt for 16 h. The reaction was quenched with water (50 mL), a short path condenser then fitted and the pale yellow mixture was heated (115° C.) in vacuo to remove the DMF (60-90° C.). The reaction volume was reduced to −300 mL and then partitioned between water (2.5 L) and EtOAc (2×500 mL) with the pH of the aqueous phase ˜8. The aqueous phase was separated and then extracted with 4:1 DCM-IPA (8×500 mL). The combined DCM-IPA phase was dried (Na₂SO₄), the drying agent filtered off and the filtrate concentrated in vacuo to yield a semi-solid residue. The crude residue was stirred in EtOH (130 mL) at 55° C. for 1 h, filtered off, the solid washed with EtOH and dried in vacuo to afford a white solid (55.7 g, 38%, regioisomer ratio 86:14). This material was re-subjected to a hot slurry in EtOH (100 mL at 55° C.), hot filtered, the solid washed with cold EtOH to give the desired compound i8 as a white crystalline solid (47.22 g): ¹H NMR (400 MHz, DMSO-d₆) δ 8.30 (s, 1H), 8.08 (s, 1H), 7.33 (br s, 2H), 7.06 (d, J=8.6 Hz, 2H), 6.73 (d, J=8.6 Hz, 2H), 6.03 (d, J=6.3 Hz, 1H), 5.46 (dd, J=7.3, 4.4 Hz, 1H), 5.28 (d, J=5.1 Hz, 1H), 4.57 (d, J=11.6 Hz, 1H), 4.53 (dd, J=6.4, 5.0 Hz, 1H), 4.37 (d, J=11.6 Hz, 1H), 4.33 (dd, J=5.0, 2.9 Hz, 1H), 4.02 (q, J=3.3 Hz, 1H), 3.69 (s, 3H), 3.67 (m, 1H), 3.56 (m, 1H); LCMS (Method B) Rt 1.86 mins; m/z 388.0 (M+H⁺).

Step 2:

Preparation of (2R,3R,4R,5R)-4-((4-methoxybenzyl)oxy)-5-(6-(tritylamino)-9H-purin-9-yl)-2-((trityloxy)methyl)tetrahydrofuran-3-ol (i9): To compound i8 (45.5 g, 117 mmol) in DMF (310 mL) was added 2,6-lutidine (68.4 mL, 587 mmol), DMAP (3.59 g, 29.4 mmol) and trityl chloride (82 g, 294 mmol). The reaction mixture was slowly heated to 80° C. The reaction mixture was stirred for 15 h at 80° C. and then cooled to rt. The reaction was poured into aq. sat. NH₄Cl (1500 mL) and extracted with EtOAc (3×1 L). The combined organic phases were dried (Na₂SO₄), the drying agent filtered off and the filtrate concentrated in vacuo. The crude product was purified by chromatography on silica gel (gradient elution EtOAc-Heptane 0-100%) to yield the desired compound i9 as an off white solid (85.79 g): ¹H NMR (400 MHz, CDCl₃) δ 8.01 (s, 1H), 7.87 (s, 1H), 7.41 (m, 12H), 7.28 (m, 18H), 7.18 (d, J=8.6 Hz, 2H), 6.95 (s, 1H), 6.80 (d, J=8.6 Hz, 2H), 6.11 (d, J=4.4 Hz, 1H), 4.77-4.67 (m, 2H), 4.62 (d, J=11.6 Hz, 1H), 4.32 (q, J=5.3 Hz, 1H), 4.21 (m, 1H), 3.79 (s, 3H), 3.49 (dd, J=10.5, 3.3 Hz, 1H), 3.36 (dd, J=10.5, 4.5 Hz, 1H), 2.66 (d, J=5.7 Hz, 1H); LCMS (Method G) Rt 1.53 mins; m/z 872.0 (M+H⁺).

Step 3:

Preparation of (2R,4S,5R)-4-((4-methoxybenzyl)oxy)-5-(6-(tritylamino)-9H-purin-9-yl)-2-((trityloxy) methyl)dihydrofuran-3(2H)-one (i10): To a solution of Dess-Martin Periodinane (DMP, 3.04 g, 7.17 mmol) in DCM (72 mL) at rt was added tert-butanol (0.713 mL, 7.45 mmol) and sodium carbonate (0.134 g, 1.261 mmol), followed by a dropwise addition over 1 h of a solution of compound i9 (5.00 g, 5.73 mmol) in DCM (72 mL). The resulting reaction mixture was stirred at rt for 4 h before additional DCM (110 mL) was added. After a further 3 h additional DMP (0.63 g) and DCM (50 mL) were added. The reaction stirred for 13 h and then quenched by addition of sat. Na₂S₂O₅ (40 mL), sat. NaHCO₃ (150 mL) and brine (50 mL). The organic phase was separated and the aqueous phase then re-extracted with DCM (2×150 mL). The combined DCM was dried (Na₂SO₄), the drying agent filtered off and the filtrate concentrated in vacuo. The crude material was purified by chromatography on silica gel (gradient elution EtOAc/heptane (0-80%) to afford compound i10 as a white foam (4.36 g): ¹H NMR (400 MHz, CDCl₃) b 7.95 (s, 1H), 7.78 (s, 1H), 7.46-7.15 (m, 30H), 7.05 (d, J=8.6 Hz, 2H), 6.98 (s, 1H), 6.73 (d, J=8.6 Hz, 2H), 6.13 (d, J=7.8 Hz, 1H), 5.23 (dd, J=7.9, 0.8 Hz, 1H), 4.80 (d, J=11.8 Hz, 1H), 4.72 (d, J=11.8 Hz, 1H), 4.35 (ddd, J=4.0, 2.4, 0.8 Hz, 1H), 3.76 (s, 3H), 3.52 (dd, J=10.5, 4.0 Hz, 1H), 3.43 (dd, J=10.5, 2.4 Hz, 1H); LCMS (Method C) Rt 1.53 mins; m/z 870.0 (M+H⁺).

Step 4:

Preparation of (2R,3S,4R,5R)-4-((4-methoxybenzyl)oxy)-5-(6-(tritylamino)-9H-purin-9-yl)-2-((trityloxy)methyl)tetrahydrofuran-3-ol (i11): To a solution of compound i10 (98 mg, 0.113 mmol) in DCM (3 mL) at −20° C. was added glacial AcOH (0.15 mL) followed by NaBH₄ (13 mg, 0.34 mmol). After 1 h the reaction mixture was quenched with 5% brine (20 mL) and extracted with EtOAc (25 mL). The organic phase was separated and dried (Na₂SO₄), the drying agent filtered off and the filtrate concentrated in vacuo to a white solid. The crude solid (3S:3R ratio 7:1) was slurried in hot MeOH (3 mL, warmed to 50° C.) with DCM (˜0.5 mL) added dropwise and the suspension cooled. The mother liquor was decanted off and the solid was dried in vacuo (63 mg, 3S:3R ratio 13:1). Recrystallization from MeOH:DCM (4 mL, v/v 5:1) gave compound i11 as a single diastereomer (ratio 50:1): ¹H NMR (400 MHz, CDCl₃) δ 7.90 (s, 1H), 7.74 (s, 1H), 7.48-7.13 (m, 32H), 6.95-6.84 (m, 2H), 5.80 (s, 1H), 4.68 (d, J 11.3 Hz, 1H), 4.49 (d, J 11.3 Hz, 1H), 4.36 (s, 1H), 4.33-4.27 (m, 1H), 4.23 (d, J 3 Hz, 1H), 3.83 (s, 3H), 3.59-3.52 (m, 2H); LCMS (Method H) Rt 1.76 mins; m/z 872.2 (M+H)⁺.

Step 5:

Preparation of 9-((2R,3S,4R,5R)-4-fluoro-3-((4-methoxybenzyl)oxy)-5-((trityloxy)methyl)tetrahydro-furan-2-yl)-N-trityl-9H-purin-6-amine (i12): To a solution of compound i11 (240 mg, 0.275 mmol) in anhydrous DCM (15 mL) at 0° C. was added anhydrous pyridine (0.223 mL, 2.75 mmol). After 5 min, diethylaminosulfur trifluoride (DAST, 0.182 mL, 1.38 mmol) was added dropwise. After 5 min, the cooling bath was removed and the reaction stirred for 4.5 h. The reaction mixture was diluted with chloroform (20 mL), dry silica gel was added, and the mixture concentrated in vacuo before adding toluene (20 mL) and concentrating to dryness in vacuo. The crude material was purified by chromatography on silica gel (gradient elution 10-50% EtOAc/heptane) to give the desired compound i12 as a white solid (121 mg): ¹H NMR (400 MHz, CDCl₃) δ 7.93 (s, 1H), 7.82 (s, 1H), 7.42-7.20 (m, 30H), 7.13-7.05 (m, 3H), 6.74 (d, J 8.3 Hz, 2H), 6.09-6.05 (m, 1H), 5.15-5.06 (m, 1H), 5.00 (dd, J54.4, and 4.4 Hz, 1H), 4.60-4.50 (m, 2H), 4.49-4.39 (m, 1H), 3.77 (s, 3H), 3.51-3.38 (m, 1H), 3.32 (dd, J=10.6, 4.0 Hz, 1H); ¹⁹F NMR (376.4 MHz, CDCl₃) δ −198.09; LCMS (Method I) Rt 1.27 mins; m/z 874.5 (M+H)⁺.

Step 6:

Preparation of (2R,3S,4S,5R)-2-(6-amino-9H-purin-9-yl)-4-fluoro-5-(hydroxymethyl)tetrahydrofuran-3-ol (i13): To a solution of compound i12 (70 mg, 0.080 mmol) in DCM (1 mL) was added TFA (0.5 mL, 6.49 mmol). After 45 min the reaction mixture was diluted with MeOH (10 mL) and concentrated in vacuo. The crude material was dissolved in MeOH (10 mL) and TEA (0.1 mL) was added before silica gel was added and the suspension concentrated in vacuo. The crude material was purified by chromatography on silica gel (gradient elution 0-10% MeOH/DCM) to give the desired compound i13 as a white solid (21 mg) containing TEA. TFA salt and used as is: ¹H NMR (400 MHz, Methanol-d₄) δ 8.33 (s, 1H), 8.21 (s, 1H), 6.02 (d, J7.9 Hz, 1H), 5.12 (dd, J 54.5, 4.3 Hz, 1H), 4.96 (ddd, J 25.1, 8.0, 4.3 Hz, 1H), 4.44 (dt, J 27.6, 2.5 Hz, 1H), 3.94-3.69 (m, 2H); ¹⁹F NMR (376.4 MHz, Methanol-d₄) δ −200.02; LCMS (Method G) Rt 0.51 mins; m/z 270.1 (M+H)⁺.

Step 7:

Preparation of N-(9-((2R,3S,4S,5R)-4-fluoro-3-hydroxy-5-(hydroxymethyl)tetrahydrofuran-2-yl)-9H-purin-6-yl)benzamide (i14): To compound i13 (3.88 g, 14.41 mmol) in pyridine (65 mL) at 0° C. was added benzoyl chloride (8.36 mL, 72.1 mmol) slowly followed by TMSCl (9.21 mL, 72.1 mmol). The reaction mixture was stirred while warming to rt for 4 h. After another 1 h the solution was quenched with water (35 mL), followed by conc. NH₄OH (17 mL) after 5 min resulting in a pale tan solid. The mixture was diluted with water (100 mL) and extracted with MeTHF (3×75 mL). The combined organic phases were dried (Na₂SO₄), the drying agent filtered off and the filtrate concentrated in vacuo to a tan semi-solid crude material, which was purified by chromatography on silica gel (gradient elution 0-20% MeOH/DCM) to give the desired compound i14 (2.75 g): ¹H NMR (400 MHz, CDCl₃) δ 8.78 (s, 1H), 8.09 (s, 1H), 8.08-8.01 (m, 2H), 7.66 (t, J=7.4 Hz, 1H), 7.57 (t, J=7.5 Hz, 2H), 6.13 (br s, 1H), 5.92 (d, J=7.9 Hz, 1H), 5.41-5.11 (m, 2H), 4.60 (d, J=28.4 Hz, 1H), 4.13-3.98 (m, 2H), 3.86 (d, J=13.0 Hz, 1H). 19F NMR (376.4 MHz, CDCl₃) δ −199.36; LCMS (Method G) Rt 0.72 mins; m/z 374.2 (M+H)⁺.

Step 8:

Preparation of N-(9-((2R,3S,4S,5R)-5-((bis(4-methoxyphenyl)(phenyl)methoxy)methyl)-4-fluoro-3-hydroxytetrahydrofuran-2-yl)-9H-purin-6-yl)benzamide (i6): To compound i14 (2.73 g, 10.14 mmol) in pyridine (55 mL) was added DMTCI (4.12 g, 12.17 mmol) in one portion. The reaction was stirred at rt for 72 h before the yellowish solution was quenched by addition of MeOH (20 mL) and then concentrated in vacuo to a semi-solid following addition of toluene (2×50 mL) to azeotrope residual pyridine. The resulting material was dissolved in DCM (100 mL), washed with sat. NaHCO₃ (100 mL), brine then dried (Na₂SO₄). The drying agent was filtered off and the filtrate evaporated in vacuo. The resulting residue was purified by chromatography on silica gel (gradient elution 0-10% MeOH/DCM with 0.04% TEA) to give compound i6 as a white solid (3.70 g): ¹H NMR (400 MHz, CDCl₃) δ 9.16 (s, 1H), 8.64 (s, 1H), 8.23 (s, 1H), 7.99 (d, J7.5 Hz, 2H), 7.59 (t, J7.4 Hz, 1H), 7.48 (t, J7.6 Hz, 2H), 7.41-7.31 (m, 3H), 7.31-7.11 (m, 7H), 6.79 (d, J8.9 Hz, 4H), 6.16 (d, J7.3 Hz, 1H), 5.77 (br s, 1H), 5.27-5.10 (m, 2H), 4.53 (dt, J28.0 Hz, 3.4 Hz, 1H), 3.77 (s, 6H), 3.51 (dd, J 10.7, 3.7 Hz, 1H), 3.34 (dd, J 10.7, 3.3 Hz, 1H); ¹⁹F NMR (376.4 MHz, CDCl₃) δ −197.5; ¹³C NMR (101 MHz, CDCl₃) δ 164.66, 158.64, 158.62, 152.60, 151.43, 149.34, 144.22, 141.66, 135.29, 135.13, 133.40, 132.93, 129.96, 128.87, 127.99, 127.93, 127.86, 127.07, 122.65, 113.26, 93.85, 92.02, 87.56 (d, J 144 Hz), 83.56 (d, J 23 Hz), 77.30, 74.63 (d, J 16 Hz), 62.82 (d, J 11 Hz), 55.26; LCMS (Method C) Rt 2.72 mins; m/z 676.3 (M+H)⁺.

Note: The LCMS or HRMS data in this example, and where indicated in the following examples, were recorded using the indicated methods as follows. In all instances, masses reported are those of the protonated parent ions unless indicated otherwise.

Method A: LCMS data were recorded using a Waters System: Micromass ZQ mass spectrometer; Column: Sunfire C18 3.5 micron, 3.0×30 mm; gradient: 40-98% MeCN in water with 0.05% TFA over a 2.0 min period; flow rate 2 mL/min; column temperature 40° C.).

Method B: LCMS were recorded using a Waters System: Micromass SQ mass spectrometer; Column: Acquity UPLC BEH C18 1.7 micron, 2.1×30 mm; gradient 1% to 30% MeCN to 3.20 min then gradient: 30-98% MeCN in water with 5 mM NH₄OH over a 1.55 min period before returning to 1% MeCN at 5.19 min-total run time 5.2 min; flow rate 1 mL/min; column temperature 50° C.

Method C: LCMS were recorded using a Waters System: Micromass SQ mass spectrometer; Column: Acquity UPLC BEH C18 1.7 micron, 2.1×50 mm; gradient: 2-98% MeCN in water+5 mM NH₄OH over a 4.40 min period isocratic for 0.65 min before returning to 2% MeCN at 5.19 min−total run time 5.2 min; flow rate 1 mL/min; column temperature 50° C.

Method E: HRMS data were recorded using a Waters System: Acquity G2 Xevo QT of mass spectrometer; Column: Acquity BEH 1.7 micron, 2.1×50 mm; gradient: 40-98% MeCN in water with 0.1% Formic acid over a 3.4 min period, isocratic 98% MeCN for 1.75 mins returning to 40% at 5.2 mins; flow rate 1 mL/min; column temperature 50° C.

Method G: LCMS data were recorded using a Waters System: Micromass SQ mass spectrometer; Column: Acquity UPLC BEH C18 1.7 micron, 2.1×30 mm; gradient 1% to 30% MeCN to 1.20 mins then gradient: 30-98% MeCN in water with 5 mM NH₄OAc over a 0.55 min period before returning to 1% MeCN at 2.19 mins—total run time 2.2 mins; flow rate 1 mL/min; column temperature 50° C.

Method H: LCMS data were recorded using a Waters System: Micromass SQ mass spectrometer; Column: Acquity UPLC BEH C18 1.7 micron, 2.1×30 mm; gradient 2% to 98% MeCN to 1.76 mins then isocratic to 2.00 mins and then returning to 2% MeCN using gradient to 2.20 mins in water with 0.1% Formic acid; flow rate 1 mL/min; column temperature 50° C.

Method I: LCMS data were recorded using a Waters System: Micromass SQ mass spectrometer; Column: Acquity UPLC BEH C18 1.7 micron, 2.1×30 mm; gradient 40% to 98% MeCN to 1.40 mins then isocratic to 2.05 mins and then returning to 40% MeCN using gradient to 2.20 mins in water with 0.1% Formic acid; flow rate 1 mL/min; column temperature 50° C.

Given the synthetic methods described above, and the synthetic methods described in WO2016/145102, WO2014/093936, WO2017/027646, WO2017/027645, WO2015/185565, WO2016/096174, WO2014/189805, US2015158886, WO2017011622, WO2017004499 and WO2007070598 the compounds listed in Tables 1-4 can be readily made.

### Linker-Drug Moiety (L-(D)ₘ)

### Linker

As used herein, a “linker” is any chemical moiety that is capable of linking an antibody, antibody fragment (e.g., antigen binding fragments) or functional equivalent to another moiety, such as a drug moiety, (e.g. a cyclic dinucleotide or cyclic dinucleoside), which binds to Stimulator of Interferon Genes (STING) receptor.

Linkers of the immunoconjugates of the invention may comprise one or more cleavage elements and in certain embodiments the linkers of the immunoconjugates of the invention comprise two or more cleavage elements, wherein each cleavage element is independently selected from a self-immolative spacer and a group that is susceptible to cleavage (such as a group which is susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage).

In some aspects, the linker is a procharged linker, a hydrophilic linker, or a dicarboxylic acid based linker.

Acid-labile linkers are linkers cleavable at acidic pH. For example, certain intracellular compartments, such as endosomes and lysosomes, have an acidic pH (pH 4-5), and provide conditions suitable to cleave acid-labile linkers.

Some linkers can be cleaved by peptidases, i.e., peptidase cleavable linkers. Only certain peptides are readily cleaved inside or outside cells, see e.g., Trout et al., 79 Proc. Natl. Acad. Sci. USA, 626-629 (1982) and Umemoto et al. 43 Int. J. Cancer, 677-684 (1989). Furthermore, peptides are composed of α-amino acids and peptidic bonds, which chemically are amide bonds between the carboxylate of one amino acid and the amino group of a second amino acid. Other amide bonds, such as the bond between a carboxylate and the ε-amino group of lysine, are understood not to be peptidic bonds and are considered non-cleavable.

Some linkers can be cleaved by esterases, i.e., esterase cleavable linkers. Again, only certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

Cleavable linkers, such as those containing a hydrazone, a disulfide, and a dipeptide (e.g. Val-Cit), are well known in the art, and can be used. See, e.g., Ducry, et al., Bioconjugate Chem. vol. 21, 5-13 (2010).

In addition, cleavable linkers containing a glucuronidase-cleavable moiety, are well known in the art, and can be used. See, e.g., Ducry, et al., Bioconjugate Chem., vol. 21, 5-13 (2010).

For the immunoconjugates of the invention comprising a cleavable linker, the linker is substantially stable in vivo until the immunoconjugate binds to or enters a cell, at which point either intracellular enzymes or intracellular chemical conditions (pH, reduction capacity) cleave the linker to free the Drug moiety.

Procharged linkers are derived from charged cross-linking reagents that retain their charge after incorporation into an antibody drug conjugate. Examples of procharged linkers can be found in US 2009/0274713.

The linker (L) can be attached to the antibody, antigen binding fragment or their functional equivalent at any suitable available position on the antibody, antigen binding fragment or their functional equivalent: typically, linker (L) is attached to an available amino nitrogen atom (i.e., a primary or secondary amine, rather than an amide) or a hydroxylic oxygen atom, or to an available sulfhydryl, such as on a cysteine.

The linker (L) of the immunoconjugates of the invention can be divalent, where the linker is used to link only one drug moiety per linker to an antibody, antigen binding moiety or functional equivalent, or the linker (L) of the immunoconjugates of the invention can be trivalent and is able to link two drug moieties per linker to an antibody, antigen binding moiety or functional equivalent. In addition, the linker (L) of in the immunoconjugates of the invention can also polyvalent and is able to link multiple drug moieties per linker to an antibody, antigen binding moiety or functional equivalent.

The linker (L) of the immunoconjugates of the invention is a linking moiety comprising one or more linker components. Some preferred linkers and linker components are described herein.

A linker component of linker (L) of the immunoconjugates of the invention can be, for example,
a) an alkylene group: —(CH₂)ₙ— (where in this instance is n is 1-18);
b) an alkenyl group;
c) an alkynyl group;
d) an ethylene glycol unit: —CH₂CH₂O—;
e) an polyethylene glycol unit: (—CH₂CH₂O—)ₓ (where x in this instance is 2-20);
f) —O—;
g) —S—;
h) a carbonyl: —C(═O)—;
i) an ester: —C(═O)—O— or —O—C(═O)—;
j) a carbonate: —OC(═O)O—;
k) an amine: —NH—;
l) an amides: —C(═O)—NH—, —NH—C(═O)— or —C(═O)N(C₁₋₆alkyl)-;
m) a carbamate: —OC(═O)NH— or —NHC(═O)O—;
n) a urea: —NHC(═O)NH—;
o) an alkylene substituted with one or more groups independently selected from carboxy, sulfonate, hydroxyl, amine, amino acid, saccharide, phosphate and phosphonate);
p) an C₁-C₁₀alkylene in which one or more methylene groups is replace by one or more —S—, —NH— or —O— moieties;
q) a ring systems having two available points of attachment such as a divalent ring selected from phenyl (including 1,2-1,3- and 1,4-di-substituted phenyls), a C₅-C₆ heteroaryl, a C₃-C₈ cycloalkyl (including 1,1-disubstituted cyclopropyl, cyclobutyl, cyclopentyl or cyclohexyl, and 1,4-disubstituted cyclohexyl), and a C₄-C₈ heterocycloalkyl;
r) a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine;
   a combination of 2 or more amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit;
and
s) a self-immolative spacer, wherein the self-immolative spacer comprises
   i. one or more protecting (triggering) groups which are susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage
   and
   ii. one or more groups which can undergo 1,4-elimination, 1,6-elimination, 1,8-elimination, 1,6-cyclization elimination, 1,5-cyclization elimination, 1,3-cyclization elimination, intramolecular 5-exo-trig or 6-exo-trig cyclization,
Non-limiting examples of such self-immolative spacer include:

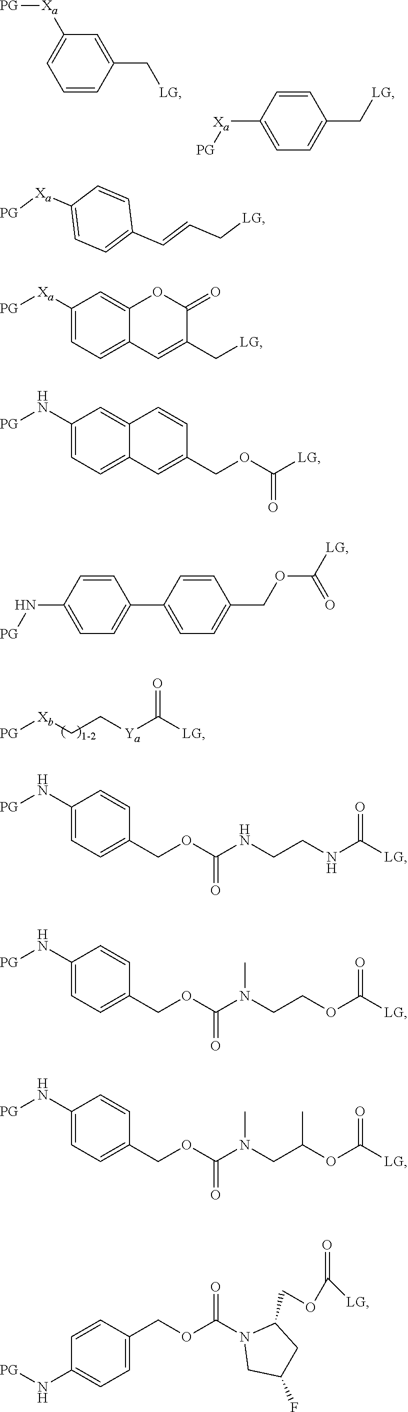

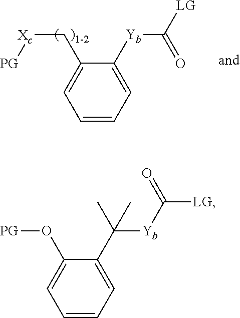

where:
   PG is a protecting (triggering) group;
   Xₐ is O, NH or S;
   X_{b} is O, NH, NCH₃ or S;
   X_{c} is O or NH;
   Yₐ is CH₂, O or NH;
   Y_{b} is a bond, CH₂, O or NH, and LG is a leaving group such as a Drug moiety (D) of the immunoconjugates of the invention.
Additional non-limiting examples of such self-immolative spacers are described n Angew. Chem. Int. Ed. 2015, 54, 7492-7509.
By way of example only, certain self-immolative spacers used in the immunoconjugates of the invention are

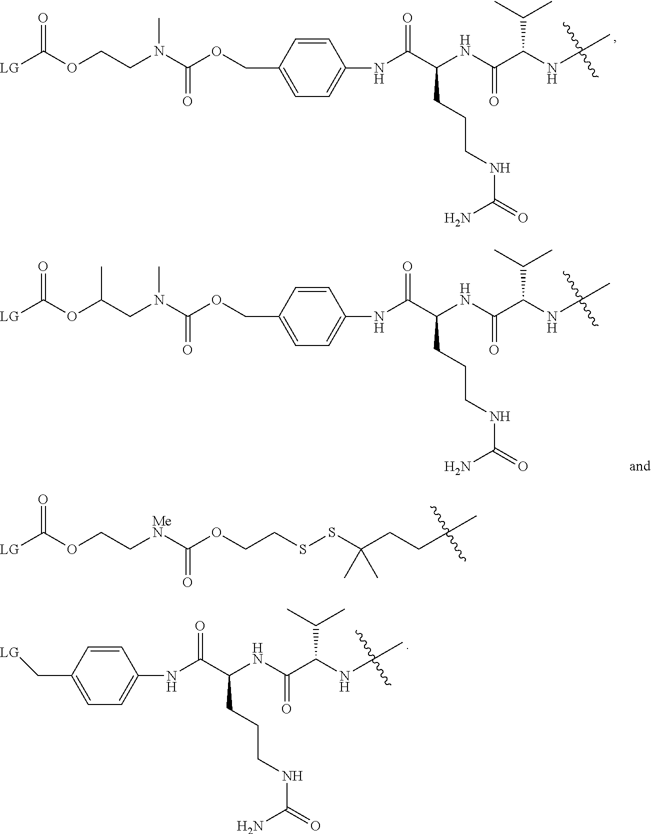

In addition, a linker component can be a chemical moiety which is readily formed by reaction between two reactive groups. Non-limiting examples of such chemical moieties are given in Table 5.

TABLE 5

| Reactive Group | Reactive Group | |
|---|---|---|
| 1 | 2 | Chemical Moiety |
| a thiol | a thiol | —S—S— |
| a thiol | a maleimide | 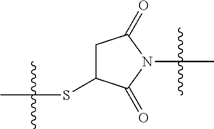 [SMILES: *SC1CC(=O)N(*)C1=O] |
| a thiol | a haloacetamide | 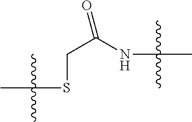 [SMILES: *NC(=O)CS*] |
| an azide | an alkyne | 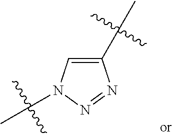 [SMILES: *c1cn(*)nn1] |
| | | 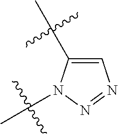 [SMILES: *c1cnnn1*] |
| an azide | a triaryl phosphine | 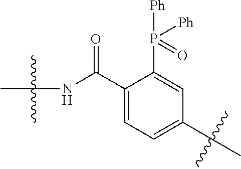 [SMILES: *NC(=O)c1ccc(*)cc1P(=O)(c1ccccc1)c1ccccc1] |
| an azide | a cyclooctyne | 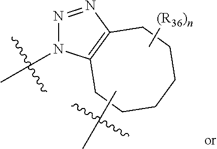 |
| | | 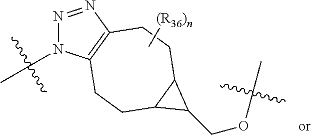 |
| | | 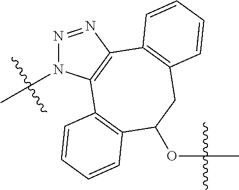 [SMILES: *OC1Cc2ccccc2-c2nnn(*)c2-c2ccccc21] |
| an azide | an oxanobornadiene | 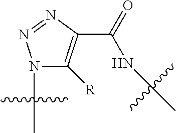 |
| a triaryl phosphine | an azide | 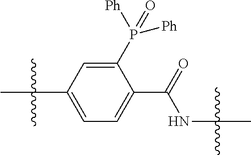 [SMILES: *NC(=O)c1ccc(*)cc1P(=O)(c1ccccc1)c1ccccc1] |
| an oxanobornadiene | an azide | 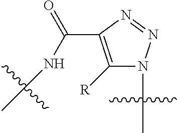 |
| an alkyne | an azide | 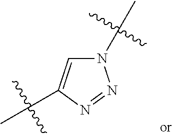 [SMILES: *c1cn(*)nn1] |
| | | 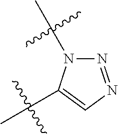 [SMILES: *c1cnnn1*] |
| a cyclooctyne | azide | 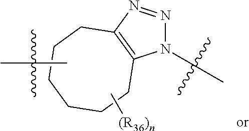 |
| | | 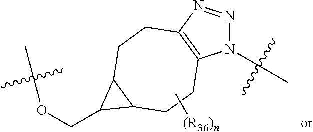 |
| | | 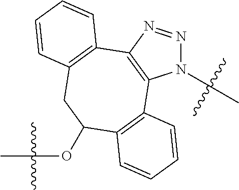 [SMILES: *OC1Cc2ccccc2-c2nnn(*)c2-c2ccccc21] |
| a cyclooctene | a diaryl tetrazine | 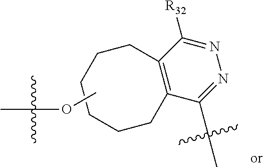 |
| | | 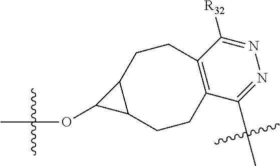 |
| a diaryl tetrazine | a cyclooctene | 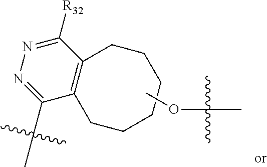 |
| | | 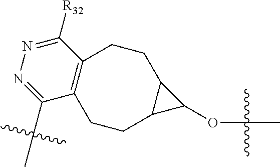 |
| a monoaryl tetrazine | a norbornene | 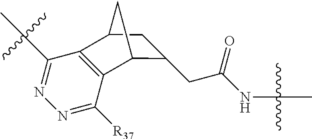 |
| a norbornene | a monoarl tetrazine | 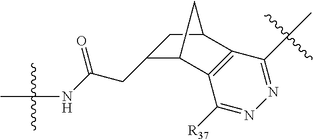 |
| an aldehyde | a hydroxylamine | 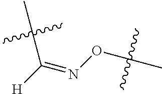 [SMILES: */C=N\O*] |
| an aldehyde | a hydrazine | 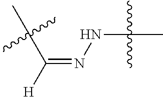 [SMILES: */C=N\N*] |
| an aldehyde | NH₂—NH—C(═O)— | 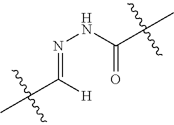 [SMILES: */C=N/NC(*)=O] |
| a ketone | a hydroxylamine | 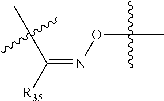 |
| a ketone | a hydrazine | 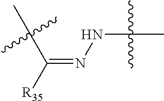 |
| a ketone | NH₂—NH—C(═O)— | 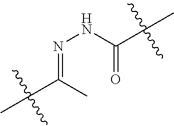 [SMILES: *C(=O)N/N=C(/*)C] |
| a hydroxylamine | an aldehyde | 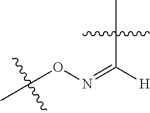 [SMILES: */C=N\O*] |
| a hydroxylamine | a ketone | 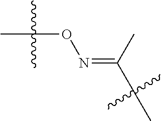 [SMILES: *O/N=C(/*)C] |
| a hydrazine | an aldehyde | 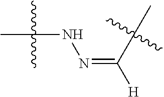 [SMILES: */C=N\N*] |
| a hydrazine | a ketone | 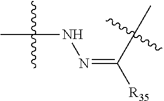 |
| NH₂—NH—C(═O)— | an aldehyde | 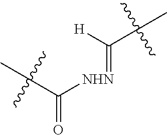 |
| NH₂—NH—C(═O)— | a ketone | 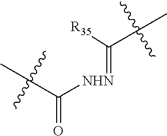 |
| a haloacetamide | a thiol | 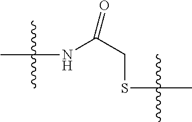 [SMILES: *NC(=O)CS*] |
| a maleimide | a thiol | 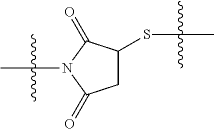 [SMILES: *SC1CC(=O)N(*)C1=O] |
| a vinyl sulfone | a thiol | 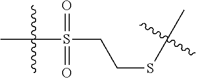 [SMILES: *SCCS(*)(=O)=O] |
| a thiol | a vinyl sulfone | 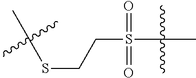 [SMILES: *SCCS(*)(=O)=O] |
| an aziridine | a thiol | 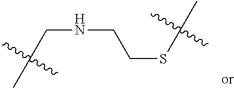 [SMILES: *CNCCS*] |
| | | 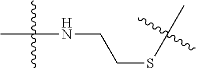 [SMILES: *NCCS*] |
| a thiol | an aziridine | 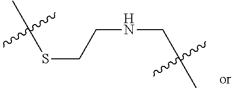 [SMILES: *CNCCS*] |
| | | 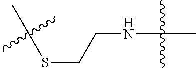 [SMILES: *NCCS*] |
| 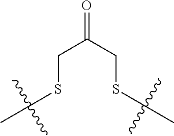 [SMILES: *SCC(=O)CS*] | hydroxylamine | 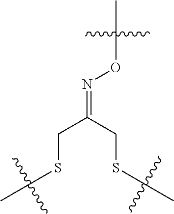 [SMILES: *ON=C(CS*)CS*] |
| 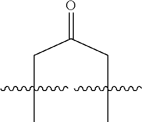 [SMILES: *CC(=O)C*] | hydroxylamine | 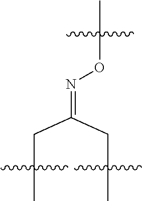 [SMILES: *CC(C*)=NO*] |
| 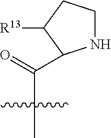 | 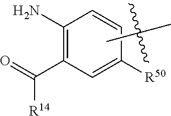 | 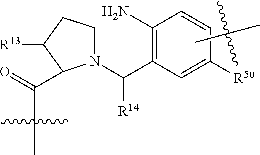 |
| 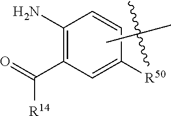 | 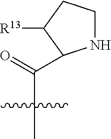 | 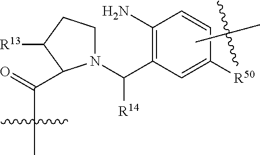 |
| 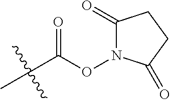 [SMILES: *C(=O)ON1C(=O)CCC1=O] | —NH₂, | amide |
| 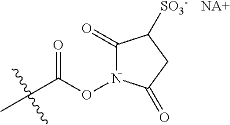 | | |
| 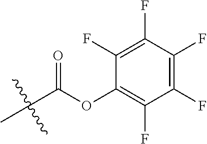 [SMILES: *C(=O)Oc1c(F)c(F)c(F)c(F)c1F] | | |
| 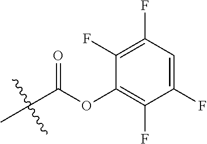 [SMILES: *C(=O)Oc1c(F)c(F)cc(F)c1F] | | |
| 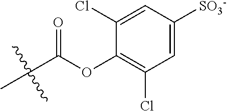 | | |
| —NH₂, | 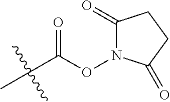 [SMILES: *C(=O)ON1C(=O)CCC1=O] | amide |
| | 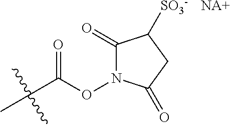 | |
| | 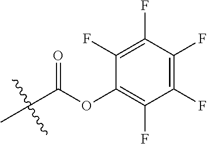 [SMILES: *C(=O)Oc1c(F)c(F)c(F)c(F)c1F] | |
| | 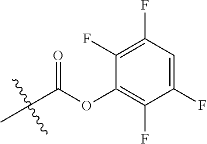 [SMILES: *C(=O)Oc1c(F)c(F)cc(F)c1F] | |
| | 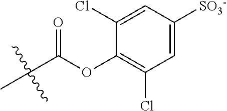 | |
| CoA or CoA analogue | Serine residue | 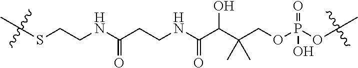 [SMILES: *OP(=O)(O)OCC(C)(C)C(O)C(=O)NCCC(=O)NCCS*] |
| | | 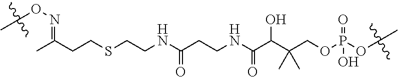 [SMILES: *O/N=C(\C)CCSCCNC(=O)CCNC(=O)C(O)C(C)(C)COP(=O)(O)O*] |
| | | 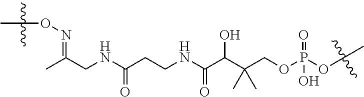 [SMILES: *O/N=C(\C)CNC(=O)CCNC(=O)C(O)C(C)(C)COP(=O)(O)O*] |
| | | 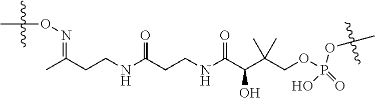 [SMILES: *O/N=C(\C)CCNC(=O)CCNC(=O)[C@H](O)C(C)(C)COP(=O)(O)O*] |
| | | 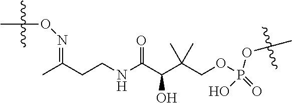 [SMILES: *O/N=C(\C)CCNC(=O)[C@H](O)C(C)(C)COP(=O)(O)O*] |
| | | 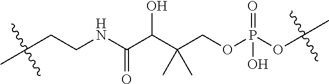 [SMILES: *CCNC(=O)C(O)C(C)(C)COP(=O)(O)O*] |
| | | 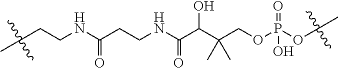 [SMILES: *CCNC(=O)CCNC(=O)C(O)C(C)(C)COP(=O)(O)O*] |
| | | 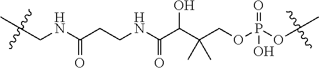 [SMILES: *CNC(=O)CCNC(=O)C(O)C(C)(C)COP(=O)(O)O*] |
| pyridyldithiol | thiol | disulfide |

where: R³² in Table 5 is H, C₁₋₄ alkyl, phenyl, pyrimidine or pyridine; R³⁵ in Table 5 is H, C₁₋₆alkyl, phenyl or C₁₋₄alkyl substituted with 1 to 3 —OH groups; each R³⁶ in Table 5 is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; R³⁷ in Table 5 is independently selected from H, phenyl and pyridine; n in Table 5 is 0, 1, 2 or 3; R¹³ in Table 5 is H or methyl; R⁵⁰ in Table 5 is H or nitro; and R¹⁴ in Table 5 is H, —CH₃ or phenyl.

In some embodiments, a linker component of linker, L, of the immunoconjugates of the invention is a group formed upon reaction of a reactive functional group with a side chain of an amino acid residue commonly used for conjugation, e.g., the thiol of a cysteine residue, or the free —NH₂ of a lysine residue. In other embodiments a linker component of linker, L, of the immunoconjugates of the invention is a group formed upon reaction of a reactive functional group with a side chain of an amino acid residue of an non-naturally occurring amino acid, such as para-acetyl Phe or para-azido-Phe. In other embodiments a linker component of linker, L, of the immunoconjugates of the invention is a group formed upon reaction of a reactive functional group with a side chain of an amino acid residue which has been engineered into the antibody, antigen binding fragment or their functional equivalent, e.g. the thiol of a cysteine residue, the hydroxyl of a serine residue, the pyrroline of a pyrrolysine residue or the pyrroline of a desmethyl pyrrolysine residue engineered into an antibody. See e.g., Ou, et al., PNAS 108(26), 10437-42 (2011).

A linker component formed by reaction with the thiol of a cysteine residue of the antibody, antigen binding fragment or their functional equivalent includes, but are not limited to,

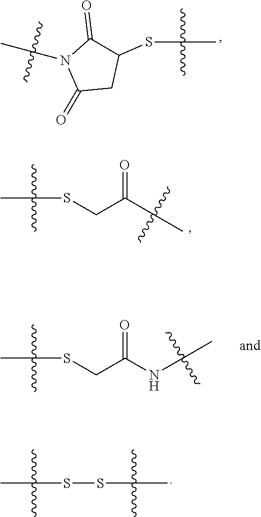

A linker components formed by reaction with the amine of a lysine residue of the antibody, antigen binding fragment or their functional equivalent include, but are not limited to,

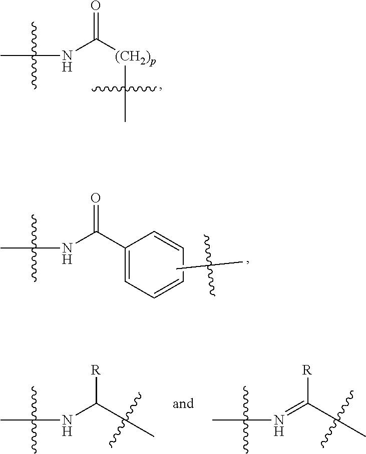

wherein each p is 1-10, and each R is independently H or C1-4 alkyl (preferably methyl).

A linker component formed by reaction with a pyrrolysine residue or desmethyl pyrrolysine residue includes, but are not limited to,

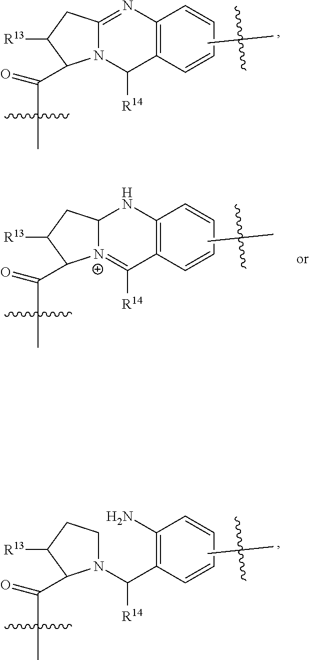

wherein R¹³ is H or methyl, and R¹⁴ is H, methyl or phenyl.

In some embodiments, a linker component of linker, L, of immunoconjugates of the invention is

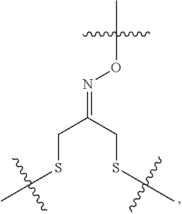 [SMILES: *ON=C(CS*)CS*]

which is formed upon reaction of a hydroxylamine and a

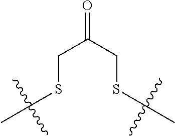 [SMILES: *SCC(=O)CS*]

moiety, where the

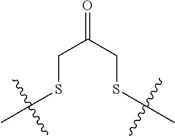 [SMILES: *SCC(=O)CS*]

moiety is formed by reduction of an interchain disulfide bridge of the antibody and re-bridging using a 1,3-dihaloacetone (e.g. 1,3-dichloroacetone, 1,3-dibromoacetone, 1,3-diiodoacetone) and bissulfonate esters of 1, 3-dihydroxyacetone. In some embodiments, a linker component of linker, L, of immunoconjugates of the invention is

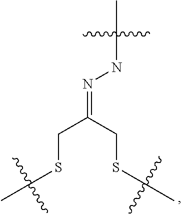 [SMILES: *NN=C(CS*)CS*]

which is formed upon reaction of a hydrazine and a

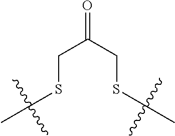 [SMILES: *SCC(=O)CS*]

moiety, where the

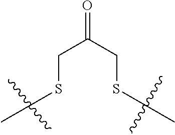 [SMILES: *SCC(=O)CS*]

moiety is formed by reduction of an interchain disulfide bridge of the antibody and re-bridging using a 1,3-dihaloacetone (e.g. 1,3-dichloroacetone, 1,3-dibromoacetone, 1,3-diiodoacetone) and bissulfonate esters of 1, 3-dihydroxyacetone.

In some embodiments, a linker component of linker, L, of immunoconjugates of the invention is selected from the groups shown in Table 6 below:

TABLE 6

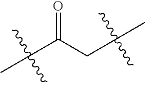 [SMILES: *CC(*)=O]

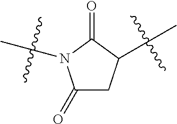 [SMILES: *C1CC(=O)N(*)C1=O]

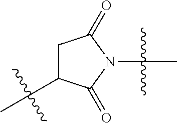 [SMILES: *C1CC(=O)N(*)C1=O]

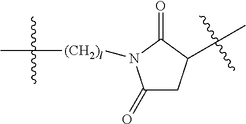

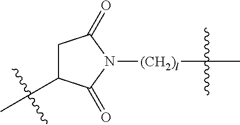

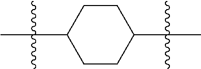 [SMILES: *C1CCC(*)CC1]

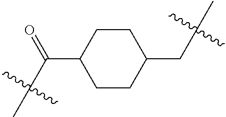 [SMILES: *CC1CCC(C(*)=O)CC1]

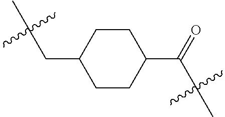 [SMILES: *CC1CCC(C(*)=O)CC1]

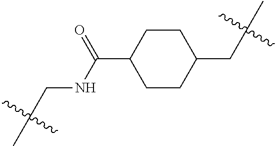 [SMILES: *CNC(=O)C1CCC(C*)CC1]

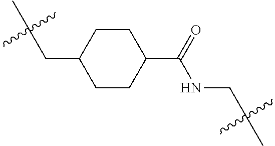 [SMILES: *CNC(=O)C1CCC(C*)CC1]

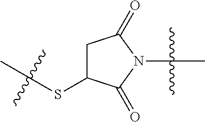 [SMILES: *SC1CC(=O)N(*)C1=O]

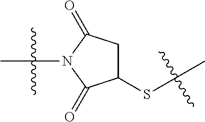 [SMILES: *SC1CC(=O)N(*)C1=O]

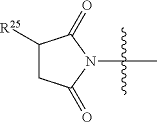

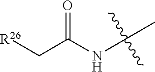

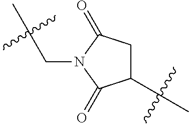 [SMILES: *CN1C(=O)CC(*)C1=O]

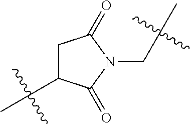 [SMILES: *CN1C(=O)CC(*)C1=O]

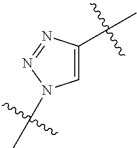 [SMILES: *c1cn(*)nn1]

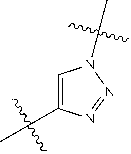 [SMILES: *c1cn(*)nn1]

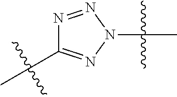 [SMILES: *c1nnn(*)n1]

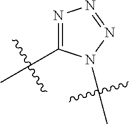 [SMILES: *c1nnnn1*]

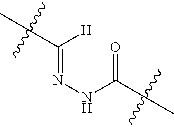 [SMILES: */C=N/NC(*)=O]

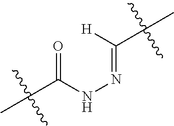 [SMILES: */C=N/NC(*)=O]

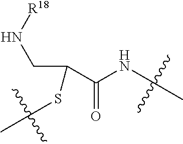

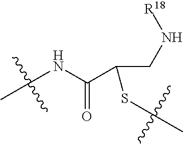

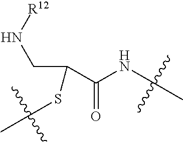

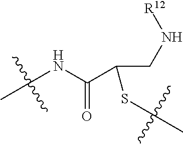

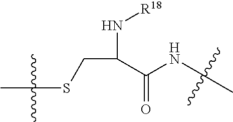

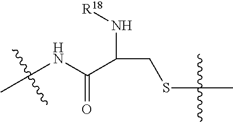

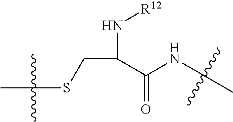

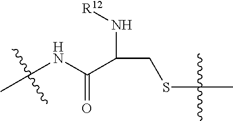

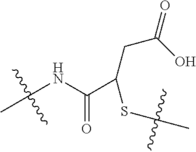 [SMILES: *NC(=O)C(CC(=O)O)S*]

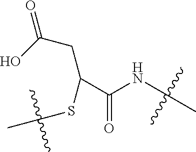 [SMILES: *NC(=O)C(CC(=O)O)S*]

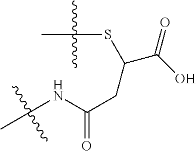 [SMILES: *NC(=O)CC(S*)C(=O)O]

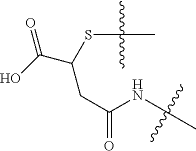 [SMILES: *NC(=O)CC(S*)C(=O)O]

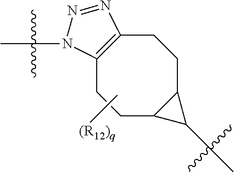

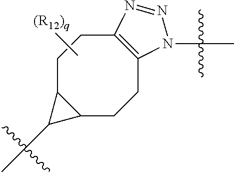

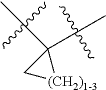 [SMILES: *C1(*)CC1]

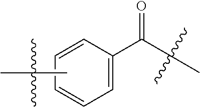

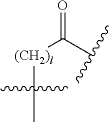

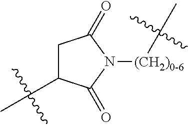 [SMILES: *CN1C(=O)CC(*)C1=O]

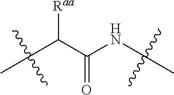

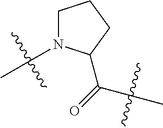 [SMILES: *C(=O)C1CCCN1*]

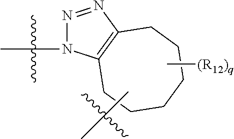

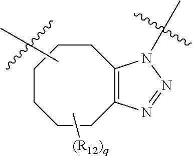

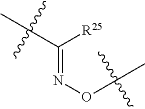

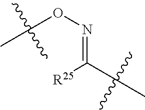

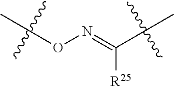

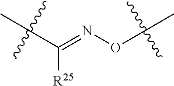

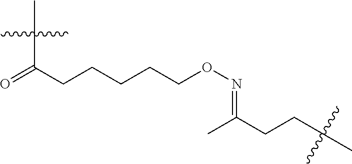 [SMILES: *CC/C(C)=N/OCCCCCC(*)=O]

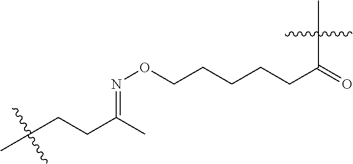 [SMILES: *CC/C(C)=N/OCCCCCC(*)=O]

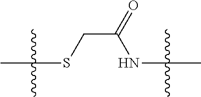 [SMILES: *NC(=O)CS*]

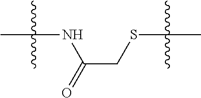 [SMILES: *NC(=O)CS*]

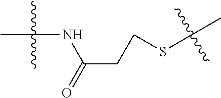 [SMILES: *NC(=O)CCS*]

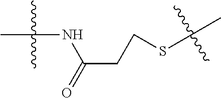 [SMILES: *NC(=O)CCS*]

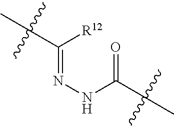

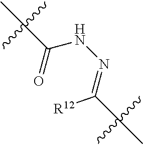

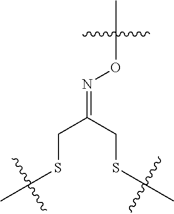 [SMILES: *ON=C(CS*)CS*]

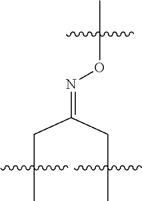 [SMILES: *CC(C*)=NO*]

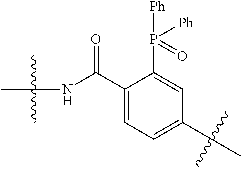 [SMILES: *NC(=O)c1ccc(*)cc1P(=O)(c1ccccc1)c1ccccc1]

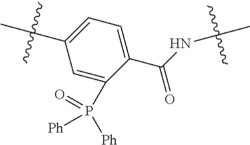 [SMILES: *NC(=O)c1ccc(*)cc1P(=O)(c1ccccc1)c1ccccc1]

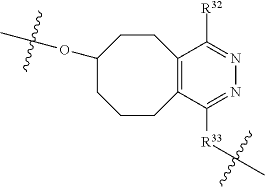

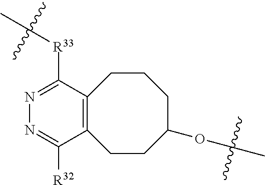

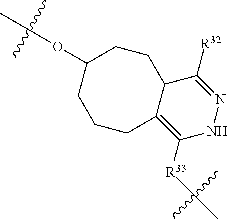

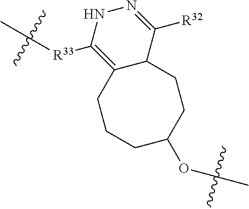

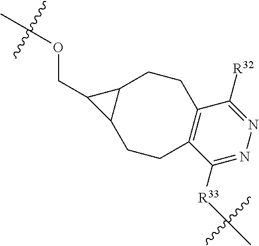

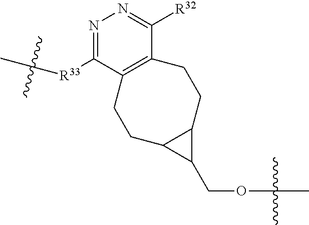

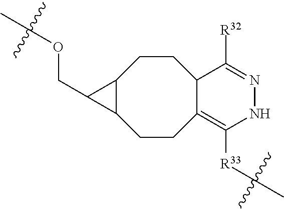

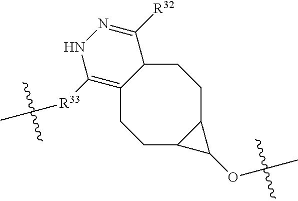

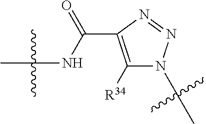

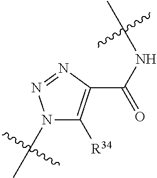

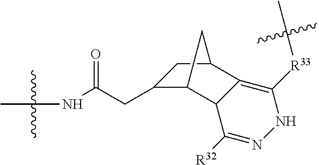

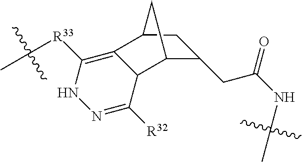

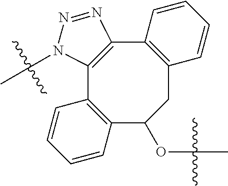 [SMILES: *OC1Cc2ccccc2-c2nnn(*)c2-c2ccccc21]

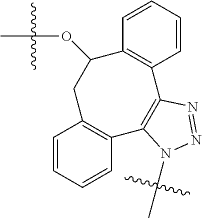 [SMILES: *OC1Cc2ccccc2-c2c(nnn2*)-c2ccccc21]

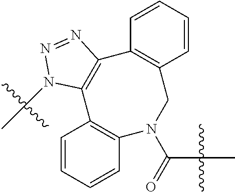 [SMILES: *C(=O)N1Cc2ccccc2-c2nnn(*)c2-c2ccccc21]

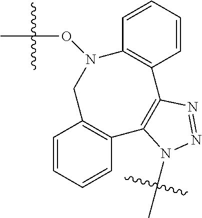 [SMILES: *ON1Cc2ccccc2-c2c(nnn2*)-c2ccccc21]

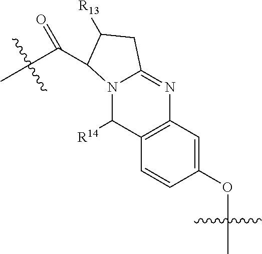

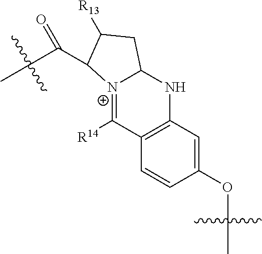

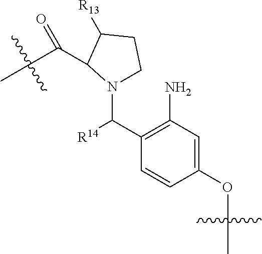

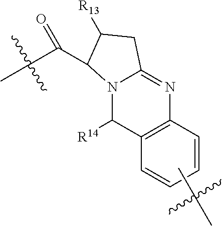

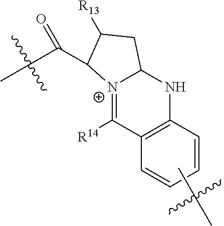

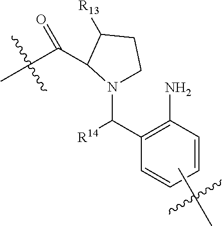

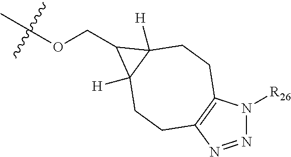

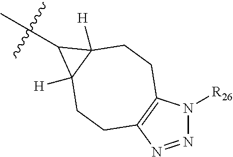

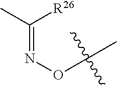

each R¹² is independently selected from H and C₁-C₆alkyl
R¹³ is H or methyl;
R¹⁴ is H, —CH₃ or phenyl;
each R²⁵ is independently selected from H or C₁₋₄ alkyl;
each R¹⁸ is independently selected from a C₁-C₆alkyl, a C₁-C₆alkyl which is substituted with azido and a C₁-C₆alkyl which is substituted with 1 to 5 hydroxyl;
I is 1, 2, 3, 4, 5 or 6;
R²⁶ is 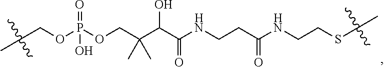 [SMILES: *COP(=O)(O)OCC(C)(C)C(O)C(=O)NCCC(=O)NCCS*] 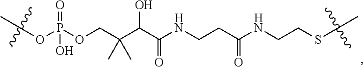 [SMILES: *OP(=O)(O)OCC(C)(C)C(O)C(=O)NCCC(=O)NCCS*] 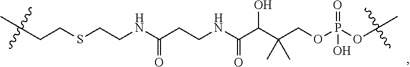 [SMILES: *CCSCCNC(=O)CCNC(=O)C(O)C(C)(C)COP(=O)(O)O*] 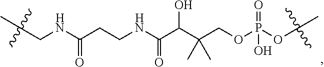 [SMILES: *CNC(=O)CCNC(=O)C(O)C(C)(C)COP(=O)(O)O*] 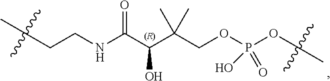 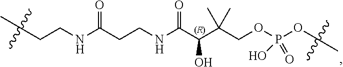 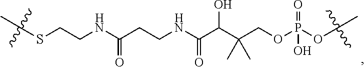 [SMILES: *OP(=O)(O)OCC(C)(C)C(O)C(=O)NCCC(=O)NCCS*] 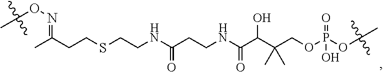 [SMILES: *O/N=C(\C)CCSCCNC(=O)CCNC(=O)C(O)C(C)(C)COP(=O)(O)O*] 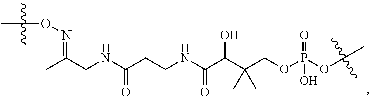 [SMILES: *O/N=C(\C)CNC(=O)CCNC(=O)C(O)C(C)(C)COP(=O)(O)O*] 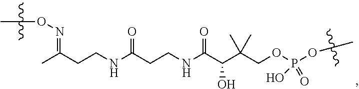 [SMILES: *O/N=C(\C)CCNC(=O)CCNC(=O)[C@@H](O)C(C)(C)COP(=O)(O)O*] 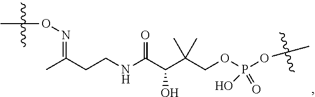 [SMILES: *O/N=C(\C)CCNC(=O)[C@@H](O)C(C)(C)COP(=O)(O)O*] 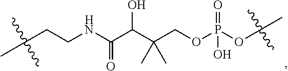 [SMILES: *CCNC(=O)C(O)C(C)(C)COP(=O)(O)O*] 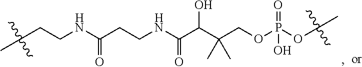 [SMILES: *CCNC(=O)CCNC(=O)C(O)C(C)(C)COP(=O)(O)O*] 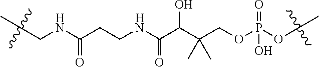 [SMILES: *CNC(=O)CCNC(=O)C(O)C(C)(C)COP(=O)(O)O*]
R³² is independently selected from H, C₁₋₄ alkyl, phenyl, pyrimidine and pyridine; 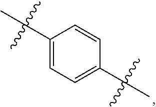 [SMILES: *c1ccc(*)cc1] 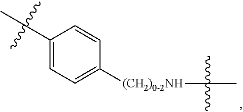
R³³ is independently selected from 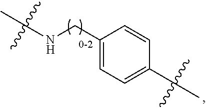 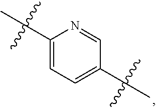 [SMILES: *c1ccc(*)nc1] 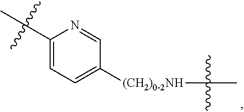 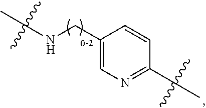 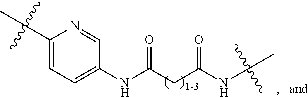 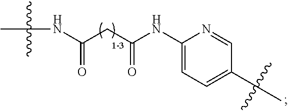
R³⁴ is independently selected from H, C₁₋₄ alkyl, and C₁₋₆ haloalkyl.

The linker, L, in the immunoconjugates of the invention typically contain two or more linker components, which may be selected for convenience in assembly of the conjugate, or they may be selected to impact properties of the conjugate.

Linkers of the immunoconjugates of the invention comprise one or more cleavage elements and in certain embodiments the linkers of the immunoconjugates of the invention comprise two or more cleavage elements. In certain embodiments one of the cleavage elements is directly attached to a Drug moiety which, after the cleavage process, allows for release of a Drug moiety which does not comprise a fragment of the cleaved linker. By way of example, the Linker-Drug Moiety (-(L-(D)ₘ)), wherein m is 1, of the immunoconjugates of the invention is designed to have one of the following structures:

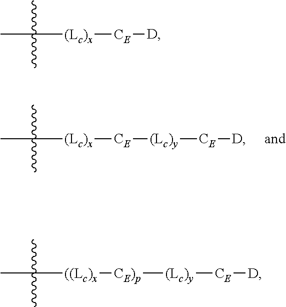

wherein:
Lc is a linker component and each Lc is independently selected from a linker component described herein;
x is an integer selected from 1, 2, 3, 4, 5, 6, 7, 8, 9, 10, 11, 12, 13, 14, 15, 16, 17, 18, 19 and 20;
y is an integer selected from 1, 2, 3, 4, 5, 6, 7, 8, 9, 10, 11, 12, 13, 14, 15, 16, 17, 18, 19 and 20;
p is an integer selected from 1, 2, 3, 4, 5, 6, 7, 8, 9 and 10;
D is a Drug moiety described herein;
and each cleavage element (C_{E}) is independently selected from a self-immolative spacer and a group that is susceptible to cleavage (such as a group which is susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage).

In one embodiment of the immunconjugates disclosed herein the Linker (L) of the Linker-Drug Moiety (-(L-(D)ₘ)), wherein m is 1, has a structure selected from:

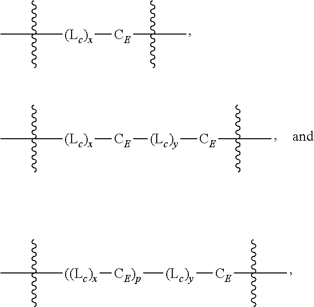

wherein:
Lc is a linker component and each Lc is independently selected from a linker component as disclosed herein;
x is an integer selected from 1, 2, 3, 4, 5, 6, 7, 8, 9, 10, 11, 12, 13, 14, 15, 16, 17, 18, 19 and 20;
y is an integer selected from 1, 2, 3, 4, 5, 6, 7, 8, 9, 10, 11, 12, 13, 14, 15, 16, 17, 18, 19 and 20;
p is an integer selected from 1, 2, 3, 4, 5, 6, 7, 8, 9 and 10;
and each cleavage element (C_{E}) is independently selected from a self-immolative spacer and a group that is susceptible to cleavage selected from acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.
   The presence of a non-cleavable linker fragment attached directly to a Drug moiety described herein is observed to decrease the activity of the Drug moiety as tested in a hSTING wt assay and THP1-dual assay (see below for description of assays and Table 7 for results), therefore such linker designs allow for the release of active Drug moieties.
   hSTING Wt Assay:

HEK-293T cells were reverse transfected with a mixture of human STING (accession BC047779 with Arg mutation introduced at position 232 to make the clone into human STING wild type) and a 5xISRE-mIFNb-GL4 plasmid (five interferon stimulated response elements and a minimal mouse interferon beta promoter driving expression of the firefly luciferase GL4). Cells were transfected using FuGENE transfection reagent (3:1 FuGENE:DNA ratio) by adding the FuGENE:DNA mix to HEK-293T cells in suspension and plating into 384 well plates. Cells were incubated overnight and treated with compounds. After 9-14 hours, plates were read by adding BrightGlo reagent (Promega) and reading on an Envision plate reader. The fold change over background was calculated and normalized to the fold-change induced by 2′3′-cGAMP at 50 uM. Plates were run in triplicate. EC50 values were calculated as described for the IP-10 secretion assay.

### THP1-Dual Assay:

THP1-Dual cells were purchased from Invivogen. THP1-Dual cells were plated in 384 well plates in 20 uL of tissue culture media and incubated overnight. Compounds were added the next day and incubated 16-24 hours. Lucia reporter signal was read out by adding Quantiluc reagent (Invivogen) followed by reading on an Envision plate reader. The fold change over background was calculated and normalized to the fold-change induced by 2′3′-cGAMP at 50 uM. Plates were run in triplicate. EC50 values were calculated as described for the IP-10 secretion assay.

### THP1-Dual/STING-KO Assay

Guide RNA (gRNA) oligo (TCCATCCATCCCGTGTCCCA (SEQ ID NO: 931)) for human STING was cloned into Lentivirus vector pNGx_LV_g003 and transduced into THP1-Dual_Cas9 cells. FACS sorted single clones were then cultured in 96 well cell culture plate. Each single well also contains 500 THP1-Dual parental cells as supporting cells. After 30 days 1 ug/ml puromycin was added to each well to eliminate supporting cells. Each individual THP1-Dual/STING-KO clone was tested using western blotting and NGS to confirm loss of STING expression and non-sense nucleotide insertion/deletion in both alleles. Six confirmed clones were then pooled and tested with cGAMP, T1-1, T1-2, using the methods described in the THP1-Dual assay above.

TABLE 7

| | | | | THP1 |
|---|---|---|---|---|
| | | | THP1 | Dual |
| | | hSTING | Dual 384 | STING |
| Compound | Structure | wt (uM) | (uM) | KO (uM) |
| T1-1 | 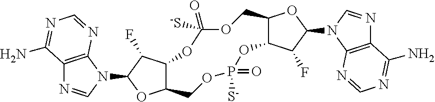 | 0.294 | 0.462 | >50 |
| | 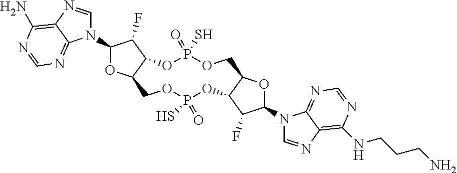 [SMILES: NCCCNc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F] | >25 | ND | ND |
| T1-2 | 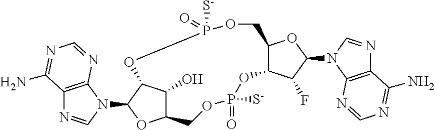 [SMILES: Nc1ncnc2c1ncn2[C@@H]1O[C@@H]2CO[P@@](=O)([S-])O[C@@H]3[C@H](O)[C@@H](CO[P@@](=O)([S-])O[C@H]2[C@H]1F)O[C@H]3n1cnc2c(N)ncnc21] | 4.48 | 1.99 | >50 |
| | 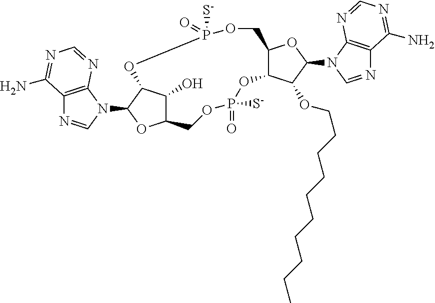 [SMILES: CCCCCCCCCCO[C@@H]1[C@@H]2O[P@](=O)([S-])OC[C@H]3O[C@@H](n4cnc5c(N)ncnc54)[C@H](O[P@](=O)([S-])OC[C@H]2O[C@H]1n1cnc2c(N)ncnc21)[C@@H]3O] | | >25 | ND |

Certain aspects and examples of the linkers and linker components of the immunoconjugates of the invention are provided in the following listing of enumerated embodiments. It will be recognized that features specified in each embodiment may be combined with other specified features to provide further embodiments of the present invention.

### Embodiment 70

A linker component of linker, L, or combinations thereof, of immunoconjugates of the invention is selected from

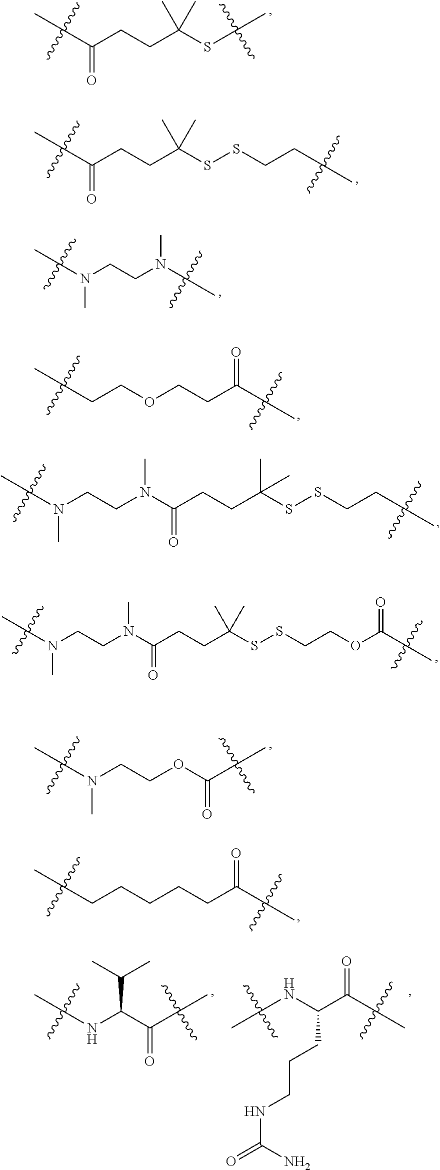

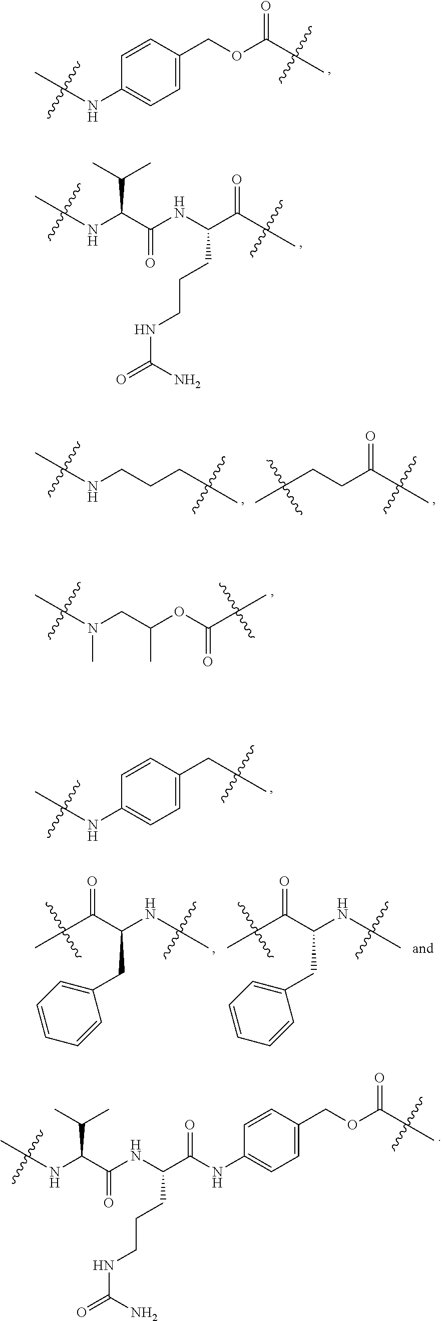

### Embodiment 71

A linker, L selected from:
—**C(═O)O(CH₂)ₘNR¹¹C(═O)(CH₂)ₘ—; —**C(═O)O(CH₂)ₘNR¹¹C(═O)(CH₂),O(CH₂)ₘ—; —**C(═O)O(CH₂)ₘNR¹¹C(═O)X₁X₂C(═O)(CH₂)ₘ—; —**C(═O)OC(R¹²)₂(CH₂)ₘNR¹¹C(═O)X₁X₂C(═O)(CH₂)ₘ—; —**C(═O)O(CH₂)ₘNR¹¹C(═O)X₁X₂C(═O)(CH₂)ₘO(CH₂)ₘ—; —**C(═O)O(CH₂)ₘNR¹¹C(═O)X₁X₂C(═O)(CH₂)ₘO(CH₂)ₘC(═O)—; —**C(═O)O(CH₂)ₘNR¹¹C(═O)X₄C(═O)NR¹¹(CH₂)ₘNR¹¹C(═O)(CH₂)ₘO(CH₂)ₘ—; —**C(═O)O(CH₂)ₘNR¹¹C(═O)X₆C(═O)(CH₂)ₘNR¹¹C(═O)(CH₂)ₘ—; —**C(═O)X₄C(═O)NR¹¹(CH₂)ₘNR¹¹C(═O)(CH₂)ₘO(CH₂)ₘ—; —**C(═O)(CH₂)ₘNR¹¹C(═O)X₁X₂C(═O)(CH₂)ₘ—; —**C(═O)O(CH₂)ₘX₆C(═O)X₁X₂C(═O)(CH₂)ₘ—; —**C(═O)(CH₂)ₘNR¹¹C(═O)((CH₂)ₘO)ₙ(CH₂)ₘ—; —**C(═O)O(CH₂)ₘX₆C(═O)(CH₂)ₘ—; —**C(═O)O(CH₂)ₘX₆C(═O)(CH₂)ₘO(CH₂)ₘ—; —**C(═O)O(CH₂)ₘX₆C(═O)X₁X₂C(═O)(CH₂)ₘ—; —**C(═O)O(CH₂)ₘX₆C(═O)X₁X₂C(═O)(CH₂)ₘO(CH₂)ₘ—; —**C(═O)O(CH₂)ₘX₆C(═O)X₁X₂C(═O)(CH₂)ₘO(CH₂)ₘC(═O)—; —**C(═O)O(CH₂)ₘX₆C(═O)X₄C(═O)NR¹¹(CH₂)ₘNR¹¹C(═O)(CH₂)ₘO(CH₂)ₘ—; —**C(═O)X₄C(═O)X₆(CH₂)ₘNR¹¹C(═O)(CH₂)ₘO(CH₂)ₘ—; —**C(═O) (CH₂)ₘX₆C(═O)X₁X₂C(═O) (CH₂)ₘ—; —**C(═O)O((CH₂)ₘO)ₙ(CH₂)ₘNR¹¹C(═O)X₆C(═O)(CH₂)ₘ—; —**C(═O)O((CH₂)ₘO)ₙ(CH₂)ₘNR¹¹C(═O)X₆C(═O)(CH₂)ₘNR¹¹C(═O)(CH₂)ₘ—; —**C(═O)O((CH₂)ₘO)ₙ(CH₂)ₘNR¹¹C(═O)X₅C(═O)(CH₂)ₘX₃(CH₂)ₘ—; —**C(═O)O((CH₂)ₘO)ₙ(CH₂)ₘNR¹¹C(═O)X₅C(═O)((CH₂)ₘO)ₙ(CH₂)ₘ—; —**C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘ—; —**C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—; —**C(═O)O((CH₂)ₘO)ₙ(CH₂)ₘNR¹¹C(═O))X₅C(═O)((CH₂)ₘO)ₙ(CH₂)ₘX₃(CH₂)ₘ—; —**C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘ—; —**C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—; —**C(═O)O((CH₂)ₘO)ₙ(CH₂)ₘNR¹¹C(═O)X₅(CH₂)ₘX₃(CH₂)ₘ—; —**C(═O)O((CH₂)ₘO)ₙ(CH₂)ₘNR¹¹C(═O)X₅((CH₂)ₘO)ₙ(CH₂)ₘ—; —**C(═O)O((CH₂)ₘO)ₙ(CH₂)ₘNR¹¹C(═O)X₅((CH₂)ₘO)ₙ(CH₂)ₘNR¹¹C(═O)(CH₂)ₘ—; —**C(═O)O((CH₂)ₘO)ₙ(CH₂)ₘNR¹¹C(═O)X₅((CH₂)ₘO)ₙ(CH₂)ₘNR¹¹C(═O)(CH₂)ₘX₃(CH₂)ₘ—; —**C(═O)O((CH₂)ₘO)ₙ(CH₂)ₘNR¹¹C(═O)X₅((CH₂)ₘO)ₙ(CH₂)ₘX₃(CH₂)ₘ—; —**C(═O)O((CH₂)ₘO)ₙ(CH₂)ₘNR¹¹C(═O)X₅(CH₂)ₘNR¹¹((CH₂)ₘO)ₙ(CH₂)ₘ—; —**C(═O)O((CH₂)ₘO)ₙ(CH₂)ₘNR¹¹C(═O)X₅C(═O)(CH₂)ₘNR¹¹((CH₂)ₘO)ₙ(CH₂)ₘX₃(CH₂)ₘ—; —**C(═O)O((CH₂)ₘO)ₙ(CH₂)ₘNR¹¹C(═O)X₅(CH₂)ₘ—; —**C(═O)O((CH₂)ₘO)ₙ(CH₂)ₘNR¹¹C(═O)X₅C(═O)((CH₂)ₘO)ₙ(CH₂)ₘ—; —**C(═O)O((CH₂)ₘO)ₙ(CH₂)ₘNR¹¹C(═O)X₅(CH₂)ₘX₃(CH₂)ₘ—; —**C(═O)O(CH₂)ₘ—; —**C(═O)O((CH₂)ₘO)ₙ(CH₂)ₘ—; —**C(═O)O(CH₂)ₘNR¹¹(CH₂)ₘ—; —**C(═O)O(CH₂)ₘNR¹¹(CH₂)ₘC(═O)X₂X₁C(═O)—; —**C(═O)O(CH₂)ₘX₃(CH₂)ₘ—; —**C(═O)O((CH₂)ₘO)ₙ(CH₂)ₘX₃(CH₂)ₘ—; —**C(═O)O((CH₂)ₘO)ₙ(CH₂)ₘNR¹¹C(═O)(CH₂)ₘ—; —**C(═O)O(CH₂)ₘNR¹¹C(═O(CH₂)ₘX₃(CH₂)ₘ—; —**C(═O)O((CH₂)ₘO)ₙ(CH₂)ₘNR¹¹C(═O)(CH₂)ₘX₃(CH₂)ₘ—; —**C(═O)O((CH₂)ₘO)ₙX₃(CH₂)ₘ—; —**C(═O)O((CH₂)ₘO)ₙ(CH₂)ₘX₃(CH₂)ₘ—; —**C(═O)O((CH₂)ₘO)ₙ(CH₂)ₘC(═O)NR¹¹(CH₂)ₘ—; —**C(═O)O(CH₂)ₘC(R¹²)₂—; —**C(═O)OCH₂)ₘC(R¹²)₂SS(CH₂)ₘNR¹¹C(═O)(CH₂)ₘ—, and —**C(═O)O(CH₂)ₘC(═O)NR¹¹(CH₂)ₘ—, where the ** of L indicates point of attachment to the drug moiety (D);
wherein:
X₁ is

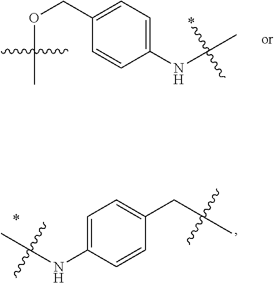

where the * of X₁ indicates the point of attachment to X₂;
X₂ is selected from

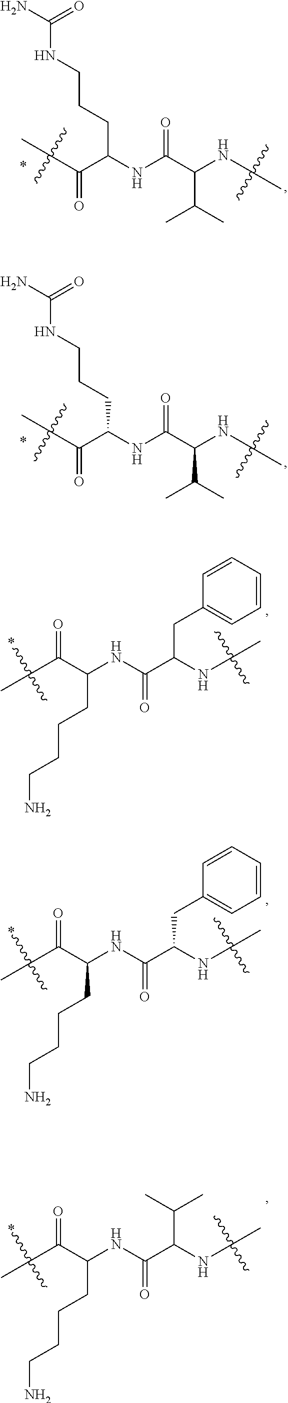 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O ; *NC(C(=O)NC(CCCCN)C(*)=O)C(C)C]

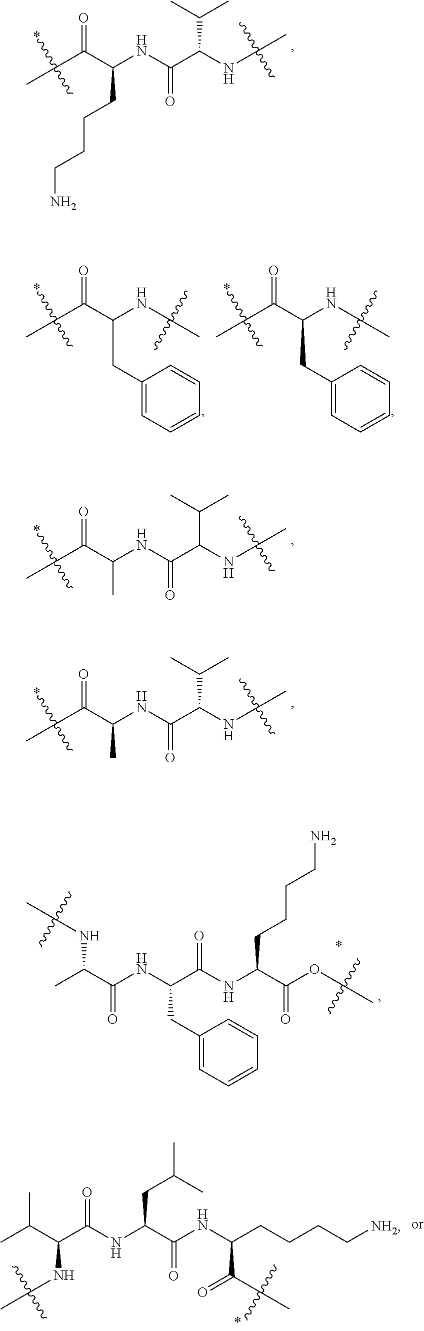

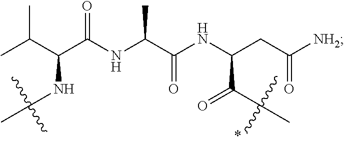 [SMILES: *N[C@H](C(=O)N[C@@H](C)C(=O)N[C@@H](CC(N)=O)C(*)=O)C(C)C]

where the * of X₂ indicates the point of attachment to X₁;
X₃ is

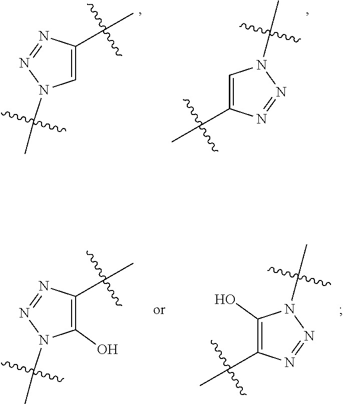 [SMILES: *c1cn(*)nn1 ; *c1cn(*)nn1 ; *c1nnn(*)c1O ; *c1nnn(*)c1O]

X₄ is —O(CH₂)ₙSSC(R¹²)₂(CH₂)ₙ— or —(CH₂)ₙC(R¹²)₂SS(CH₂)ₙO—;
X₅ is

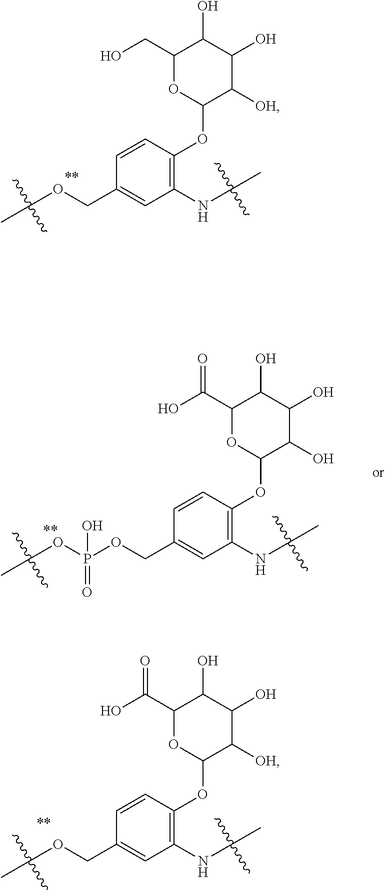 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the ** of X₅ indicates orientation toward the Drug moiety;
X₆ is

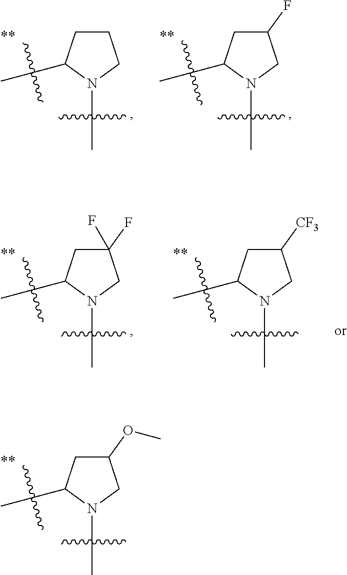

or, where the ** of X₆ indicates orientation toward the Drug moiety;
each R¹¹ is independently selected from H and C₁-C₆alkyl;
each R¹² is independently selected from H and C₁-C₆alkyl;
each m is independently selected from 1, 2, 3, 4, 5, 6, 7, 8, 9 and 10, and
each n is independently selected from 1, 2, 3, 4, 5, 6, 7, 8, 9, 10, 11, 12, 13, 14, 15, 16, 17 and 18.

### Embodiment 72

A linker, L selected from:
—**C(═O)(CH₂)ₘ—; —**C(═O)((CH₂)ₘO)ₙ(CH₂)ₘ—; —**C(═O)(CH₂)ₘNR¹¹(CH₂)ₘ—; —**C(═O)(CH₂)ₘNR¹¹(CH₂)ₘC(═O)X₂X₁C(═O)—; —**C(═O)₍CH₂)ₘX₃(CH₂)ₘ—; —**C(═O)((CH₂)ₘO)ₙ(CH₂)ₘX₃(CH₂)ₘ—; —**C(═O)(CH₂)ₘNR¹¹C(═O)(CH₂)ₘ—; —**C(═O)((CH₂)ₘO)ₙ(CH₂)ₘNR¹¹C(═O)(CH₂)ₘ—; —**C(═O)(CH₂)ₘNR¹¹C(═O(CH₂)ₘX₃(CH₂)ₘ—; —**C(═O)((CH₂)ₘO)ₙ(CH₂)ₘNR C(═O)(CH₂)ₘX₃(CH₂)ₘ—; —**C(═O)((CH₂)ₘO)ₙX₃(CH₂)ₘ—; —**C(═O)((CH₂)ₘO)ₙ(CH₂)ₘX₃(CH₂)ₘ—; —**C(═O)((CH₂)ₘO)ₙ(CH₂)ₘC(═O)NR¹¹(CH₂)ₘ—; —**C(═O)(CH₂)ₘC(R¹²)₂—; —**C(═O)((CH₂)ₘO)ₙ(CH₂)ₘNR¹¹C(═O)X₅C(═O)(CH₂)ₘ—; —**C(═O)((CH₂)ₘO)ₙ(CH₂)ₘNR¹¹C(═O)X₅C(═O)(CH₂)ₘNR¹¹C(═O)(CH₂)ₘ—; —**C(═O)((CH₂)ₘO)ₙ(CH₂)ₘNR¹¹C(═O)X₅C(═O)(CH₂)ₘX₃(CH₂)ₘ—; —**C(═O)((CH₂)ₘO)ₙ(CH₂)ₘNR¹¹C(═O)X₅C(═O)((CH₂)ₘO)ₙ(CH₂)ₘ—; —**C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘ—; —**C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—; —**C(═O)((CH₂)ₘO)ₙ(CH₂)ₘNR¹¹C(═O))X₅C(═O)((CH₂)ₘO)ₙ(CH₂)ₘX₃(CH₂)ₘ—; —**C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘ—; —**C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—; —**C(═O)((CH₂)ₘO)ₙ(CH₂)ₘNR¹¹C(═O)X₅(CH₂)ₘX₃(CH₂)ₘ—; —**C(═O)((CH₂)ₘO)ₙ(CH₂)ₘNR¹¹C(═O)X₅((CH₂)ₘO)ₙ(CH₂)ₘ—; —**C(═O)((CH₂)ₘO)ₙ(CH₂)ₘNR¹¹C(═O)X₅((CH₂)ₘO)ₙ(CH₂)ₘNR¹¹C(═O)(CH₂)ₘ—; —**C(═O)((CH₂)ₘO)ₙ(CH₂)ₘNR^{1l}C(═O)X₅((CH₂)ₘO)ₙ(CH₂)ₘNR¹¹C(═O)(CH₂)ₘX₃(CH₂)ₘ—; —**C(═O)((CH₂)ₘO)ₙ(CH₂)ₘNR¹¹C(═O)X₅((CH₂)ₘO)ₙ(CH₂)ₘX₃(CH₂)ₘ—; —**C(═O)((CH₂)ₘO)ₙ(CH₂)ₘNR¹¹C(═O)X₅(CH₂)ₘNR¹¹((CH₂)ₘO)ₙ(CH₂)ₘ—; —**C(═O)((CH₂)ₘO)ₙ(CH₂)ₘNR¹C(═O)X₅C(═O)(CH₂)ₘNR¹¹((CH₂)ₘO)ₙ(CH₂)ₘX₃(CH₂)ₘ—; —**C(═O)((CH₂)ₘO)ₙ(CH₂)ₘNR¹¹C(═O)X₅(CH₂)ₘ—; —**C(═O)((CH₂)ₘO)ₙ(CH₂)ₘNR¹¹C(═O)X₅C(═O)((CH₂)ₘO)ₙ(CH₂)ₘ—; —**C(═O)((CH₂)ₘO)ₙ(CH₂)ₘNR¹¹C(═O)X₅(CH₂)ₘX₃(CH₂)ₘ—; —**C(═O)(CH₂)ₘC(R¹²)₂SS(CH₂)ₘNR¹¹C(═O)(CH₂)ₘ—, and —**C(═O)(CH₂)ₘC(═O)NR¹¹(CH₂)ₘ—,
   where the ** of L indicates point of attachment to the drug moiety (D), and
      X₁, X₂, X₃, X_{q}, Xs, R¹¹, R¹², n and m are as defined in Embodiment 63.

### Embodiment 73

A linker, L selected from:
—**C(═O)X₁X₂C(═O)(CH₂)ₘ—; —**C(═O)X₁X₂C(═O)(CH₂)ₘNR¹¹C(═O)(CH₂)ₘ—; —**C(═O)X₁X₂C(═O)(CH₂)ₘX₃(CH₂)ₘ—; —**C(═O)X₁X₂C(═O)((CH₂)ₘO)ₙ(CH₂)ₘ—; —**C(═O)X₁X₂C(═O)((CH₂)ₘO)ₙ(CH₂)ₘNR¹¹C(═O)(CH₂)ₘ—; —**C(═O)X₁X₂C(═O)((CH₂)ₘO)ₙ(CH₂)ₘNR¹¹C(═O)(CH₂)ₘX₃(CH₂)ₘ—; —**C(═O)X₁X₂C(═O)((CH₂)ₘO)ₙ(CH₂)ₘX₃(CH₂)ₘ—; —**C(═O)X₁X₂C(═O)(CH₂)ₘNR¹¹C(═O)((CH₂)ₘO)ₙ(CH₂)ₘ—; —**C(═O)X₁X₂C(═O)(CH₂)ₘNR¹¹C(═O)((CH₂)ₘO)ₙ(CH₂)ₘX₃(CH₂)ₘ—; —**C(═O)X₁X₂(CH₂)ₘX₃(CH₂)ₘ—; —**C(═O)X₁X₂((CH₂)ₘO)ₙ(CH₂)ₘ—; —**C(═O)X₁X₂((CH₂)ₘO)ₙ(CH₂)ₘNR¹¹C(═O)(CH₂)ₘ—; —**C(═O)X₁X₂((CH₂)ₘO)ₙ(CH₂)ₘNR¹¹C(═O)(CH₂)ₘX₃(CH₂)ₘ—; —**C(═O)X₁X₂((CH₂)ₘO)ₙ(CH₂)ₘX₃(CH₂)ₘ—; —**C(═O)X₁X₂(CH₂)ₘNR¹¹((CH₂)ₘO)ₙ(CH₂)ₘ—; —**C(═O)X₁X₂C(═O)(CH₂)ₘNR¹¹((CH₂)ₘO)ₙ(CH₂)ₘX₃(CH₂)ₘ—; —**C(═O)NR¹¹(CH₂)ₘ—; —C(═O)NR¹¹(CH₂)ₘX₃(CH₂)ₘ—; —**C(═O)NR¹¹(CH₂)ₘNR¹¹C(═O)X₁X₂C(═O)(CH₂)ₘ—; —**C(═O)NR¹¹(CH₂)ₘNR¹¹C(═O)O(CH₂)ₘ—; —**C(═O)NR¹¹ (CH₂)ₘNR¹¹C(═O)X₁X₂—; —**C(═O)NR¹¹ (CH₂)ₘNR¹¹C(═O)X₅—; —**C(═O)NR¹¹(CH₂)ₘNR¹¹C(═O)(CH₂)ₘX₅(CH₂)ₘ—; —**C(═O)X₁C(═O)NR¹¹(CH₂)ₘX₅(CH₂)ₘ—; —**C(═O)NR¹¹ (CH₂)ₘNR¹¹C(═O)(CH₂)ₘ—; —**C(═O)NR¹¹(CH₂)ₘNR¹¹C(═O)(CH₂)ₘO(CH₂)ₘ—; —**C(═O)NR¹¹(CH₂)ₘNR¹¹C(═O)X₁X₂C(═O)(CH₂)ₘO(CH₂)ₘ—; —**C(═O)NR¹¹(CH₂)ₘNR¹¹C(═O)X₁X₂C(═O) (CH₂)ₘO(CH₂)ₘC(═O)—; —**C(═O)NR¹¹(CH₂)ₘNR¹¹C(═O)X₄C(═O)NR¹¹(CH₂)ₘNR¹¹C(═O)(CH₂)ₘO(CH₂)ₘ—; —**C(═O)NR¹¹(CH₂)ₘNR¹¹C(═O)X₁X₂C(═O)(CH₂)ₘ—; —**C(═O)NR¹¹(CH₂)ₘNR¹¹C(═O)X₅C(═O)(CH₂)ₘ—; —**C(═O)NR¹¹(CH₂)ₘNR¹¹C(═O)X₅C(═O)(CH₂)ₘNR¹¹C(═O)(CH₂)ₘ—; —**C(═O)NR¹¹(CH₂)ₘNR¹¹C(═O)X₅C(═O)(CH₂)ₘX₃(CH₂)ₘ—; —**C(═O)NR¹¹(CH₂)ₘNR¹¹C(═O)X₅C(═O)((CH₂)ₘO)ₙ(CH₂)ₘ—; —**C(═O)NR¹¹(CH₂)ₘNR¹¹C(═O)X₅C(═O)((CH₂)ₘO)ₙ(CH₂)ₘNR¹¹C(═O)(CH₂)ₘ—; —**C(═O)NR¹¹(CH₂)ₘNR¹¹C(═O)X₅C(═O)((CH₂)ₘO)ₙ(CH₂)ₘNR¹¹C(═O)(CH₂)ₘX₃(CH₂)ₘ—; —**C(═O)NR¹¹(CH₂)ₘNR¹¹C(═O)X₅C(═O)((CH₂)ₘO)ₙ(CH₂)ₘX₃(CH₂)ₘ—; —**C(═O)NR¹¹(CH₂)ₘNR¹¹C(═O)X₅C(═O)(CH₂)ₘNR¹¹C(═O)((CH₂)ₘO)ₙ(CH₂)ₘ—; —**C(═O)NR¹¹(CH₂)ₘNR¹¹C(═O)X₅C(═O)(CH₂)ₘNR¹¹C(═O)((CH₂)ₘO)ₙ(CH₂)ₘX₃(CH₂)ₘ; —**C(═O)NR¹¹(CH₂)ₘNR¹¹C(═O)X₅(CH₂)ₘX₃(CH₂)ₘ—; —**C(═O)NR¹¹(CH₂)ₘNR¹¹C(═O)X₅((CH₂)ₘO)ₙ(CH₂)ₘ—; —**C(═O)NR¹¹(CH₂)ₘNR¹¹C(═O)X₅((CH₂)ₘO)ₙ(CH₂)ₘNR¹¹C(═O)(CH₂)ₘ—; —**C(═O)NR¹¹(CH₂)ₘNR¹¹C(═O)X₅((CH₂)ₘO)ₙ(CH₂)ₘNR¹¹C(═O)(CH₂)ₘX₃(CH₂)ₘ—; —**C(═O)NR¹¹(CH₂)ₘNR¹¹C(═O)X₅((CH₂)ₘO)ₙ(CH₂)ₘX₃(CH₂)ₘ—; —**C(═O)NR¹¹(CH₂)ₘNR¹¹C(═O)X₅(CH₂)ₘNR¹¹ ((CH₂)ₘO)ₙ(CH₂)ₘ—; —**C(═O)NR¹¹(CH₂)ₘNR¹¹C(═O)X₅C(═O)(CH₂)ₘNR¹¹ ((CH₂)ₘO)ₙ(CH₂)ₘX₃(CH₂)ₘ—; —**C(═O)NR¹¹(CH₂)ₘNR¹¹C(═O)X₅(CH₂)ₘ—; —**C(═O)NR¹¹ (CH₂)ₘNR¹¹C(═O)X₅C(═O)((CH₂)ₘO)ₙ(CH₂)ₘ—; —**C(═O)NR¹¹(CH₂)ₘNR¹¹C(═O)X₅(CH₂)ₘX₃(CH₂)ₘ—; —**C(═O)X₁C(═O)NR¹¹ (CH₂)ₘNR¹¹C(═O)(CH₂)ₘ—; —**C(═O)X₁C(═O)NR¹¹(CH₂)ₘX₃(CH₂)ₘ—; —**C(═O)NR¹¹ (CH₂)ₘNR¹¹C(═O)(CH₂)ₘ—; —**C(═O)NR¹¹(CH₂)ₘNR¹¹C(═O)(CH₂)ₘX₃(CH₂)ₘ—; —**C(═O)NR¹¹ (CH₂)ₘNR¹¹C(═O)—; —**C(═O)X₁X₂(CH₂)ₘ—; —**C(═O)X₁X₂C(═O)((CH₂)ₘO)ₙ(CH₂)ₘ—; —**C(═O)X₁X₂(CH₂)ₘX₃(CH₂)ₘ—; —**C(═O)NR¹¹ (CH₂)ₘX₃(CH₂)ₘ—; —**C(═O)NR¹¹((CH₂)ₘO)ₙ(CH₂)ₘX₃(CH₂)ₘ—; —**C(═O)X₁X₂C(═O)((CH₂)ₘO)ₙ(CH₂)ₘ; —**C(═O)X₁X₂C(═O)(CH₂)ₘ—; —**C(═O)X₁C(═O)(CH₂)ₘNR¹¹C(═O)(CH₂)ₘ—, and —**C(═O)X₁C(═O)(CH₂)ₘNR¹¹C(═O)((CH₂)ₘO)ₙ(CH₂)ₘ—,
   where the ** of L indicates point of attachment to the drug moiety (D), and X₁, X₂, X₃, X₄, X₅,
   R¹¹, R¹², n and m are as defined in Embodiment 63.

### Embodiment 74

A linker, L selected from
—**C(═O)O(CH₂)ₘNR¹¹C(═O)(CH₂)ₘ—; —**C(═O)O(CH₂)ₘNR¹¹C(═O)(CH₂)ₘO(CH₂)ₘ—, —**C(═O)O(CH₂)ₘNR¹¹C(═O)X₁X₂C(═O)(CH₂)ₘ—; —**C(═O)OC(R²)₂(CH₂)ₘNR¹¹C(═O)X₁X₂C(═O)(CH₂)ₘ—; —**C(═O)O(CH₂)ₘNR¹¹C(═O)X₁X₂C(═O)(CH₂)ₘO(CH₂)ₘ—; —**C(═O)O(CH₂)ₘNR¹¹C(═O)X₁X₂C(═O)(CH₂)ₘO(CH₂)ₘC(═O)—; —**C(═O)O(CH₂)ₘNR¹¹C(═O)X₄C(═O)NR¹¹(CH₂)ₘNR¹¹C(═O)(CH₂)ₘO(CH₂)ₘ—; —**C(═O)O(CH₂)ₘNR¹¹C(═O)X₅C(═O)(CH₂)ₘNR¹¹C(═O)(CH₂)ₘ—; —**C(═O)O(CH₂)ₘX₆C(═O)X₁X₂C(═O)((CH₂)ₘO)ₙ(CH₂)ₘC(═O)—; **—(CH₂)ₘNR¹¹C(═O)X₁X₂C(═O)(CH₂)ₘ—, —**(CH₂)ₘ(CHOH)(CH₂)ₘNR¹¹C(═O)X₁X₂C(═O)(CH₂)ₘ; —**C(═O)X(C(═O)(CH)ₘNR¹¹C(═O)X₁X₂C(═O)(CH); —**C(═O)X₄C(═O)NR¹¹(CH₂)ₘNR¹¹C(═O)(CH₂)ₘO(CH₂)ₘ—; —**C(═O)(CH₂)ₘNR¹¹C(═O)X₁X₂C(═O)(CH₂)ₘ—; —**(CH₂)ₘNR¹¹C(═O)X₁X₂C(═O)(CH₂)ₘ—; —C(═O)O(CH₂)ₘX₆C(═O)X₁X₂C(═O)(CH₂)ₘ—**, or —C(═O)(CH₂)ₘNR¹¹C(═O)((CH₂)ₘO)ₙ(CH₂)ₘ—**;
   where the ** of L indicates point of attachment to the drug moiety (D), and X₁, X₂, X₄, R¹¹,
   R¹², n and m are as defined in Embodiment 63.

### Embodiment 75

A linker, L selected from:

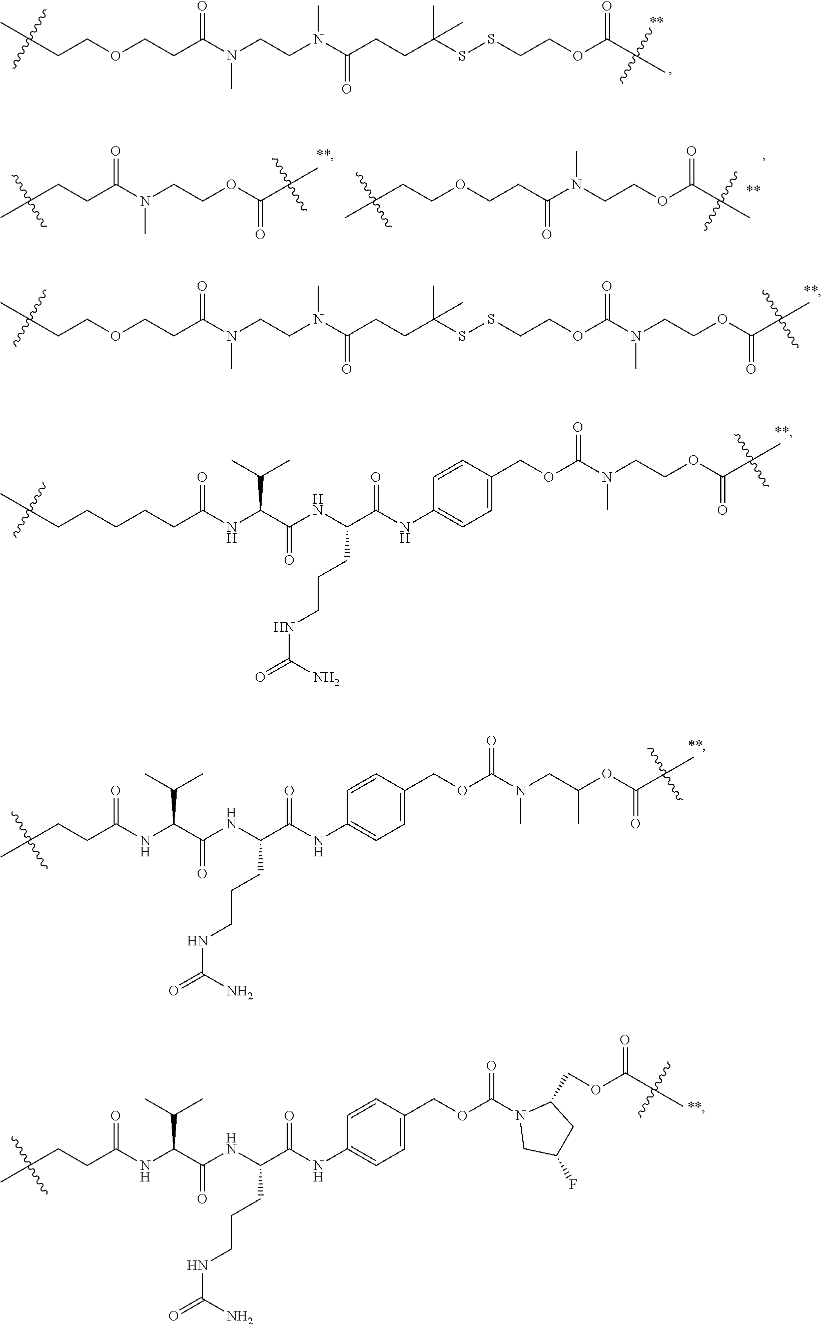

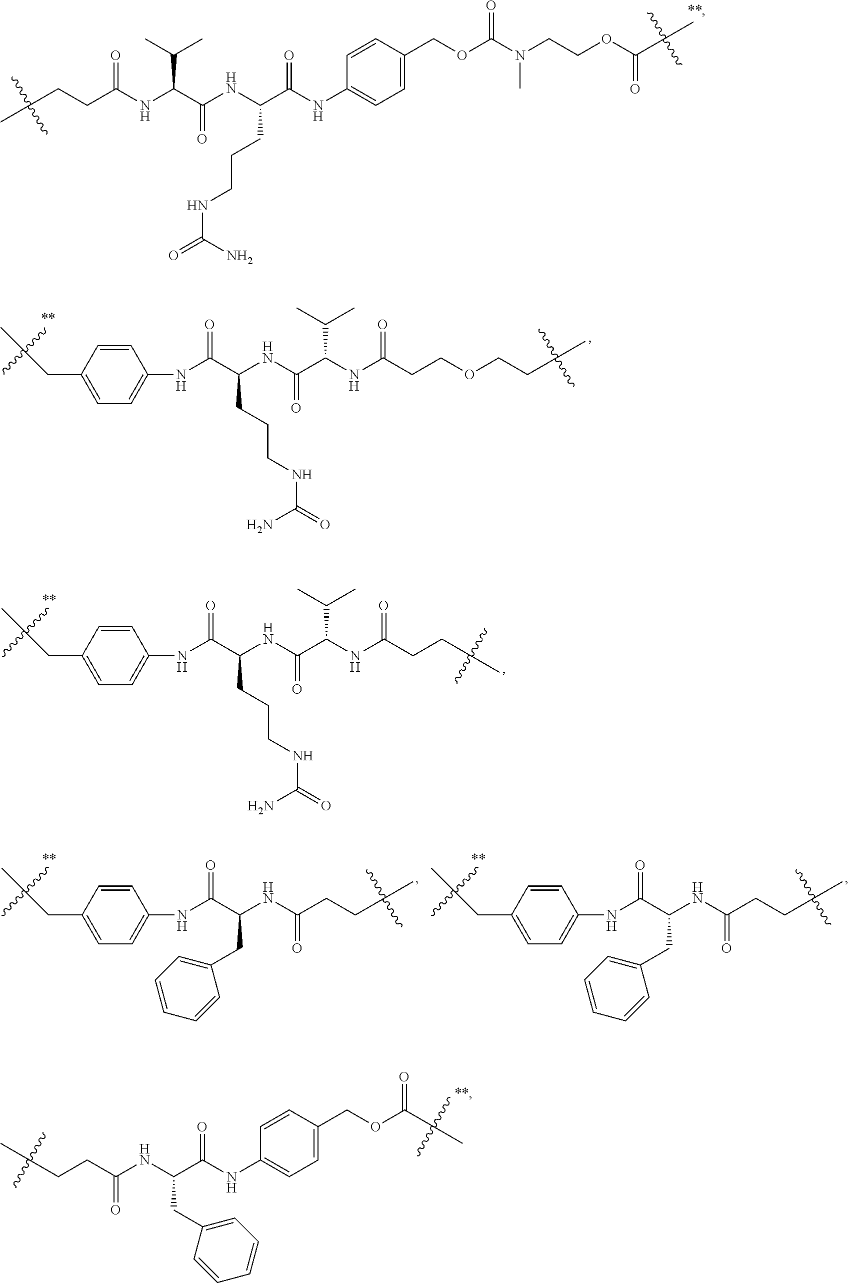

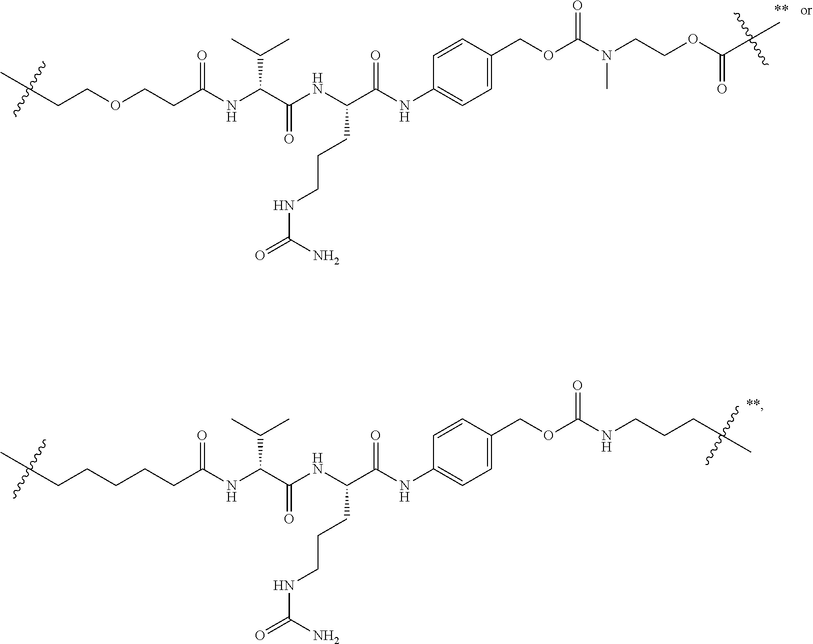 [SMILES: *CCOCCC(=O)N[C@@H](C(=O)N[C@@H](CCCNC(N)=O)C(=O)Nc1ccc(COC(=O)N(C)CCOC(*)=O)cc1)C(C)C ; *CCCCCC(=O)N[C@@H](C(=O)N[C@@H](CCCNC(N)=O)C(=O)Nc1ccc(COC(=O)NCCC*)cc1)C(C)C]

where the ** indicates the point of attachment to the drug moiety (D).

In one aspect, the Linker-Drug moiety of the immunoconjugates of the invention comprises one or more Drug moieties (D) as described herein.

In one aspect, the Linker-Drug moiety of the immunoconjugates of the invention comprises one or more Drug moieties (D), wherein the Drug moiety (D) is a compound which binds to Stimulator of Interferon Genes (STING) receptor and which comprises one or more reactive moieties capable of forming a covalent bond with a linker (L).

In one aspect, the Linker-Drug moiety of the immunoconjugates of the invention comprises one or more Drug moieties (D), wherein the Drug moiety (D) is a compound which binds to Stimulator of Interferon Genes (STING) receptor and which comprises one or more reactive moieties capable of forming a covalent bond with a linker (L), wherein linker (L) is a cleavable linker.

In one aspect, the Linker-Drug moiety of the immunoconjugates of the invention comprises one or more Drug moieties (D), wherein the Drug moiety (D) is a dinucleotide which binds to Stimulator of Interferon Genes (STING) receptor and which comprises one or more reactive moieties capable of forming a covalent bond with one or more linker(s) (L).

In one aspect, the Linker-Drug moiety of the immunoconjugates of the invention, comprises one or more Drug moieties (D), wherein the Drug moiety (D) is a dinucleotide which binds to Stimulator of Interferon Genes (STING) receptor and which comprises one or more reactive moieties capable of forming a covalent bond with one or more linker(s) (L), wherein linker (L) is a cleavable linker.

In one aspect, the Linker-Drug moiety of the immunoconjugates of the invention comprises one or more Drug moieties (D), wherein the Drug moiety (D) is a cyclic dinucleotide which binds to Stimulator of Interferon Genes (STING) receptor and which comprises one or more reactive moieties capable of forming a covalent bond with one or more linker(s) (L).

In one aspect, the Linker-Drug moiety of the immunoconjugates of the invention, comprises one or more Drug moieties (D), wherein the Drug moiety (D) is a cyclic dinucleotide which binds to Stimulator of Interferon Genes (STING) receptor and which comprises one or more reactive moieties capable of forming a covalent bond with one or more linker(s) (L), wherein linker (L) is a cleavable linker.

In one aspect the Linker-Drug moiety of the invention is a compound having the structure of Formula (A), Formula (B), Formula (C), Formula (D), Formula (E) or Formula (F) or stereoisomers or pharmaceutically acceptable salts thereof, wherein:
a) one or more linkers is attached to one or more sugar moieties of Formula (A), Formula (B), Formula (C), Formula (D), Formula (E) or Formula (F), or
b) one or more linkers is attached to one or more R¹, R^{1a} and R^{1b} groups of Formula (A), Formula (B), Formula (C), Formula (D), Formula (E) or Formula (F), or
c) one or more linkers is attached to one or more sugar moieties of Formula (A), Formula (B), Formula (C), Formula (D), Formula (E) or Formula (F) and one or more linkers is attached to one or more R¹, R^{1a} and R^{1b} groups of Formula (A), Formula (B), Formula (C), Formula (D), Formula (E) or Formula (F).

Certain aspects and examples of the Linker-Drug moiety of the invention are provided in the following listing of additional, enumerated embodiments. It will be recognized that features specified in each embodiment may be combined with other specified features to provide further embodiments of the present invention.

### Embodiment 76

A compound having the structure of Formula (A), Formula (B), Formula (C), Formula (D), Formula (E) or Formula (F), or stereoisomers or pharmaceutically acceptable salts thereof,

wherein:
each G₁ is independently selected from

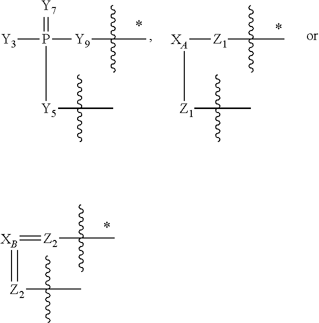

where the * of G₁ indicates the point of attachment to —CR⁸R⁹—;
X_{A} is C(═O)—, —C(═S)— or —C(═NR¹¹)— and each Z₁ is NR¹²;
X_{B} is C, and each Z₂ is N; G₂ is

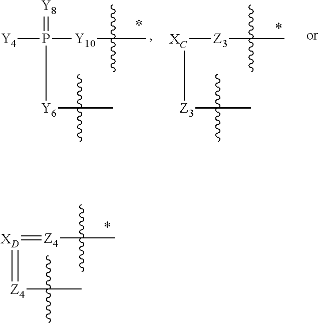

where the * of G₂ indicates the point of attachment to —CR^{8a}R^{9a}—;
X_{C} is C(═O)—, —C(═S)— or —C(═NR¹¹)— and each Z₃ is NR¹²;
X_{D} is C, and each Z₄ is N;
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₃ is OH, O⁻, OR¹⁰, N(R¹⁰)₂, SeH, Se⁻, BH₃, SH or S⁻;
Y₄ is OH, O⁻, OR¹⁰, N(R¹⁰)₂, SeH, Se⁻, BH₃, SH or S⁻;
Y₅ is —CH₂—, —NH—, —O— or —S;
Y₆ is —CH₂—, —NH—, —O— or —S;
Y₇ is O or S;
Y₈ is O or S;
Y₉ is —CH₂—, —NH—, —O— or —S;
Y₁₀ is —CH₂—, —NH—, —O— or —S;
Y₁₁ is —O—, —S—, —S(═O)—, —SO₂—, —CH₂—, or —CF₂—;
q is 1, 2 or 3;
R¹ is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R¹ is substituted with 0, 1, 2, 3 or 4 substituents independently selected from —NHL₁R¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1a} is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1a} is substituted with 0, 1, 2, 3 or 4 substituents independently selected from —NHL₁R¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
R^{1b} is a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1b} is substituted with 0, 1, 2, 3 or 4 substituents independently selected from —NHL₁R¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
each R⁸ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁸ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R⁹ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁹ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁹ are substituted by 0, 1, 2 or 3 substituents independently selected from F, Cl, Br, I, OH, CN, and N₃;
R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
R^{3a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
R^{4a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
R^{5a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
R^{7a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
R^{8a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{8a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
R^{9a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{9a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{9a} are substituted by 0, 1, 2 or 3 substituents independently selected from F, Cl, Br, I, OH, CN, and N₃;
each R¹⁰ is independently selected from the group consisting of H, C₁-C₁₂alkyl, —(CH₂CH₂O)ₙCH₂CH₂C(═O)OC₁-C₆alkyl, and

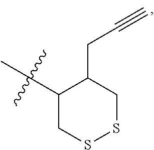 [SMILES: *C1CSSCC1CC#C]

wherein the C₁-C₁₂alkyl of R¹⁰ is substituted by 0, 1, 2 or 3 substituents independently selected from —OH, C₁-C₁₂alkoxy, —S—C(═O)C₁-C₆alkyl and C(O)OC₁-C₆alkyl;
each R¹¹ is independently selected from H and C₁-C₆alkyl;
each R¹² is independently selected from H and C₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position;
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position;
optionally R⁸ and R⁹ are connected to form a C₁-C₆alkylene, C₂-C₆alkenylene, C₂-C₆alkynylene, and
optionally R^{8a} and R^{9a} are connected to form a C₁-C₆alkylene, C₂-C₆alkenylene, C₂-C₆alkynylene,
L₁ is a linker;
R¹⁵ is a reactive group selected from any one of the groups RG1 in Table 5;
   and provided at least one of R¹, R^{1a} or R^{1b} is substituted with —NHL₁R¹⁵, or at least one of R³, R⁴, R⁵, R⁷, R^{3a}, R^{4a}, R^{5a} or R^{7a} is —OL₁R¹⁵.

### Embodiment 77

A compound of Embodiment 76, wherein L₁ is a linker comprising one or more cleavage elements.

### Embodiment 78

A compound of Formula (A-1), Formula (B-1), Formula (C-1), Formula (D-1), Formula (E-1) or Formula (F-1), or stereoisomers or pharmaceutically acceptable salts thereof, wherein R¹, R^{1a}, R^{1b}, R², R^{2a}, R³, R^{3a}, R⁴, R^{4a}, R⁵, R^{5a}, R⁶, R^{6a}, R⁷, R^{7a}, R⁸, R^{8a}, R⁹, Y₁, Y₂, Y₃, Y₄, Y₅, Y₆, Y₇, Y₈, Y₉, Y₁₀ and Y₁₁ are as described in Embodiment 76, and provided at least one of R¹, R^{1a} or R^{1b} is substituted with —NHL₁R¹⁵, or at least one of R³, R⁴, R⁵, R⁷, R^{3a}, R^{4a}, R^{5a} or R^{7a} is —OL₁R¹⁵.

### Embodiment 79

A compound of Formula (A), Formula (B), Formula (C), Formula (D), Formula (E), Formula (F), Formula (A-1), Formula (B-1), Formula (C-1), Formula (D-1), Formula (E-1) or Formula (F-1), wherein R¹ is pyrimidine or purine nucleic acid base or analogue thereof, R^{1a} is a pyrimidine or purine nucleic acid base or analogue thereof and R^{1b} is a pyrimidine or purine nucleic acid base or analogue thereof, each of which is substituted as described in R¹, R^{1a} and R^{1b} in Embodiment 76.

### Embodiment 80

A compound of Formula (A-2), Formula (B-2), Formula (C-2), Formula (D-2), Formula (E-2) or Formula (F-2), wherein R¹, R^{1a}, R^{1b}, R², R^{2a}, R³, R^{3a}, R⁴, R^{4a}, R⁵, R^{5a}R⁶, R^{6a}, R⁷, R^{7a}, R⁸, R^{8a}, R⁹, R^{9a}, Y₁, Y₂, Y₃, Y₄, Y₅, Y₆, Y₇, Y₈, Y₉, Y₁₀ and Y₁₁ are as defined in Embodiment 76, and provided at least one of R¹, R^{1a} or R^{1b} is substituted with —NHL₁R¹⁵, or at least one of R³, R⁴, R⁵, R⁷, R^{3a}, R^{4a}, R^{5a} or R^{7a} is —OL₁R¹⁵.

### Embodiment 81

A compound of Formula (A), Formula (A-1) or Formula (A-2) of any one of Embodiments 76 to 80, wherein:
R² and R^{2a} are H;
one of R³ and R⁴ is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ or R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃;
R⁷ and R^{7a} are H;
R⁶ and R^{6a} are H;
R⁸, R⁹, R^{8a} and R^{9a} are independently H or C₁-C₆alkyl, and
one of R^{3a} and R^{4a} is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃,
and provided at least one of R¹ or R^{1a} is substituted with —NHL₁R¹⁵, or at least one of R³, R⁴, R^{3a} or R^{4a} is —OL₁R¹⁵.

### Embodiment 82

A compound of Formula (A), Formula (A-1) or Formula (A-2) of any one of Embodiments 76 to 81, wherein:
Y₁ and Y₂ are O, CH₂ or S;
Y₅ and Y₆ are O or S;
Y₇ and Y₈ are O or S;
Y₉ and Y₁₀ are O or S;
R², R^{2a}, R⁶, R^{6a}, R⁷ and R^{7a} are H
one of R^{3a} and R^{4a} is H and the other is —OL₁R¹⁵, H, OH or F;
one of R³ and R⁴ is H and the other is —OL₁R¹⁵, H, OH or F; and
R^{8a}, R^{9a}, R⁸ and R⁹ are independently selected from H or C₁-C₆alkyl,
and provided at least one of R¹ or R^{1a} is substituted with —NHL₁R¹⁵, or at least one of R³, R⁴, R^{3a} or R^{4a} is —OL₁R¹⁵.

### Embodiment 83

A compound of Formula (B), Formula (B-1) or Formula (B-2) of any one of Embodiments 76 to 80, wherein:
R² and R^{2a} are H;
one of R^{3a} and R^{4a} is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
R^{7a} and R^{6a} are H;
R⁶ and R⁴ are H;
R⁸, R⁹, R^{8a} and R^{9a} are independently H or C₁-C₆alkyl, and
one of R⁵ and R⁷ is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃, and provided at least one of R¹ or R^{1a} is substituted with —NHL₁R¹⁵, or at least one of R⁵, R⁷, R^{3a} or R^{4a} is —OL₁R¹⁵.

### Embodiment 84

A compound of Formula (B), Formula (B-1) or Formula (B-2) of any one of Embodiments 76 to 80 or 83, wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R^{7a}, R^{6a}, R⁶ and R⁴ are H;
one of R^{3a} and R^{4a} is H and the other is —OL₁R¹⁵, H, OH or F;
one of R⁵ and R⁷ is H and the other is —OL₁R¹⁵, H, OH or F, and
R^{8a}, R^{9a}, R⁸ and R⁹ are independently selected from H or C₁-C₆alkyl,
and provided at least one of R¹ or R^{1a} is substituted with —NHL₁R¹⁵, or at least one of R⁵, R⁷, R^{3a} or R^{4a} is —OL₁R¹⁵.

### Embodiment 85

A compound of Formula (C), Formula (C-1) or Formula (C-2) of any one of Embodiments 76 to 80, wherein:
R² and R^{2a} are H;
one of R³ and R⁴ is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃;
R^{4a} and R^{6a} are H;
R⁶ and R⁷ are H;
R⁸, R⁹, R^{8a} and R^{9a} are independently H or C₁-C₆alkyl;
one of R^{5a} and R^{7a} is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} or R^{7a} are substituted by 0, 1, 2 or 3 substituents independently selected from F, Cl, Br, I, OH, CN, and N₃,
and provided at least one of R¹ or R^{1a} is substituted with —NHL₁R¹⁵, or at least one of R^{5a}R^{7a}, R³ or R⁴ is —OL₁R¹⁵.

### Embodiment 86

A compound of Formula (C), Formula (C-1) or Formula (C-2) of any one of Embodiments 76 to 80 or 85, wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R^{4a}, R^{6a}, R⁶ and R⁷ are H;
one of R³ and R⁴ is H and the other is —OL₁R¹⁵, H, OH or F;
one of R^{5a} and R^{7a} is H and the other is —OL₁R¹⁵, H, OH or F, and
R^{8a}, R^{9a}, R⁸ and R⁹ are independently selected from H or C₁-C₆alkyl,
and provided at least one of R¹ or R^{1a} is substituted with —NHL₁R¹⁵, or at least one of R^{5a}R^{7a}, R³ or R⁴ is —OL₁R¹⁵.

### Embodiment 87

A compound of Formula (D), Formula (D-1) or Formula (D-2) of any one of Embodiments 76 to 80, wherein:
R² and R^{2a} are H;
one of R^{5a} and R^{7a} is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} or R^{7a} are substituted by 0, 1, 2 or 3 substituents independently selected from F, Cl, Br, I, OH, CN, and N₃;
R^{4a} and R^{6a} are H;
R⁶ and R⁴ are H;
R⁸, R⁹, R^{8a} and R^{9a} are independently H or C₁-C₆alkyl, and
one of R⁵ and R⁷ is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃, and provided at least one of R¹ or R^{1a} is substituted with —NHL₁R¹⁵, or at least one of R^{5a}R^{7a}, R⁵ or R⁷ is —OL₁R¹⁵.

### Embodiment 88

A compound of Formula (D), Formula (D-1) or Formula (D-2) of any one of Embodiments 76 to 80 or 87, wherein:
Y¹ and Y² are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y⁵ and Y⁶ are O or S;
Y₇ and Y₈ are O or S;
Y⁹ and Y¹⁰ are O or S;
R², R^{2a}, R^{4a}, R^{6a}, R⁶ and R⁴ are H;
one of R^{5a}, R^{7a} is H and the other is —OL₁R¹⁵, OH or F;
one of R⁵ and R⁷ is H and the other is —OL₁R¹⁵, H, OH or F, and
R⁸, R⁹, R^{8a} and R^{9a} are independently H or C₁-C₆alkyl,
and provided at least one of R¹ or R^{1a} is substituted with —NHL₁R¹⁵, or at least one of R^{5a}R^{7a}, R⁵ or R⁷ is —OL₁R¹⁵.

### Embodiment 89

A compound of Formula (E), Formula (E-1) or Formula (E-2) of any one of Embodiments 76 to 80, wherein:
R² and R^{2a} are H;
R⁶ and R^{6a} are H;
R^{7a} is H;
R⁸, R⁹, R^{8a} and R^{9a} are independently H or C₁-C₆alkyl, and
one of R^{3a} and R^{4a} is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
one of R³ and R⁴ is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃, and
one of R⁵ and R⁷ is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃,
and provided at least one of R¹ or R^{1a} is substituted with —NHL₁R¹⁵, or at least one of R^{3a}R^{4a}, R³, R⁴, R⁵ or R⁷ is —OL₁R¹⁵.

### Embodiment 90

A compound of Formula (E), Formula (E-1) or Formula (E-2) of any one of Embodiments 76 to 80 or 89, wherein:
Y¹ and Y² are O, CH₂ or S;
Y₃ is OH, O⁻, OR¹⁰, N(R¹⁰)₂, SH or S⁻;
Y⁵ is O or S;
Y₇ and Y₈ are O or S;
Y⁹ and Y₁₀ are O or S;
R², R^{2a}, R^{5a}, R^{6a}, R⁶ and R^{7a} are H;
one of R^{3a}, R^{4a} is H and the other is —OL₁R¹⁵, H, OH, OCH₃ or F;
one of R³, R⁴ is H and the other is —OL₁R¹⁵, H, OH, OCH₃ or F;
one of R⁵ and R⁷ is H and the other is —OL₁R¹⁵, —OL₁R¹⁵, H, OH, OCH₃ or F, and
R⁸, R⁹, R^{8a} and R^{9a} are independently H or C₁-C₆alkyl,
and provided at least one of R¹ or R^{1a} is substituted with —NHL₁R¹⁵, or at least one of R^{3a}R^{4a}, R³, R⁴, R⁵ or R⁷ is —OL₁R¹⁵.

### Embodiment 91

A compound of Formula (F), Formula (F-1) or Formula (F-2) of any one of Embodiments 76 to 80, wherein:
R² and R^{2a} are H;
each R⁶ and R^{6a} are H;
each R^{7a} and R⁷ are H;
R⁸, R⁹, R^{8a} and R^{9a} are independently H or C₁-C₆alkyl, and
one of R^{3a} and R^{4a} is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
one of R³ and R⁴ is H and the other is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃, and
R⁵ is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ is substituted by 0, 1, 2 or 3 substituents independently selected from F, Cl, Br, I, OH, CN, and N₃,
and provided at least one of R¹, R^{1a} or R^{1b} is substituted with —NHL₁R¹⁵, or at least one of R^{3a}, R^{4a}, R³, R⁴, R⁵ or R⁷ is —OL₁R¹⁵.

### Embodiment 92

A compound of Formula (F), Formula (F-1) or Formula (F-2) of any one of Embodiments 76 to 80 or 91, wherein:
Y¹ and Y² are O, CH₂ or S;
each Y₃ is OH, O⁻, OR¹⁰, N(R¹⁰)₂, SH or S⁻;
each Y⁵ is O or S;
each Y₇ is independently are O or S;
each Y₉ is independently O or S;
Y₁ is O, CH₂ or S;
R², R^{2a}, R⁶, R^{6a}, R⁶, R⁷ and R^{7a} are H;
one of R^{3a}, R^{4a} is H and the other is —OL₁R¹⁵, H, OH, OCH₃ or F;
one of R³, R⁴ is H and the other is —OL₁R¹⁵, H, OH, OCH₃ or F;
R⁵ is —OL₁R¹⁵, H, OH, OCH₃ or F, and
R⁸, R⁹, R^{8a} and R^{9a} are independently H or C₁-C₆alkyl,
and provided at least one of R¹, R^{1a} or R^{1b} is substituted with —NHL₁R¹⁵, or at least one of R^{3a}, R^{4a}, R³, R⁴, R⁵ or R⁷ is —OL₁R¹⁵.

### Embodiment 93

A compound of any one of Embodiments 76 to 92 wherein:
R¹ is

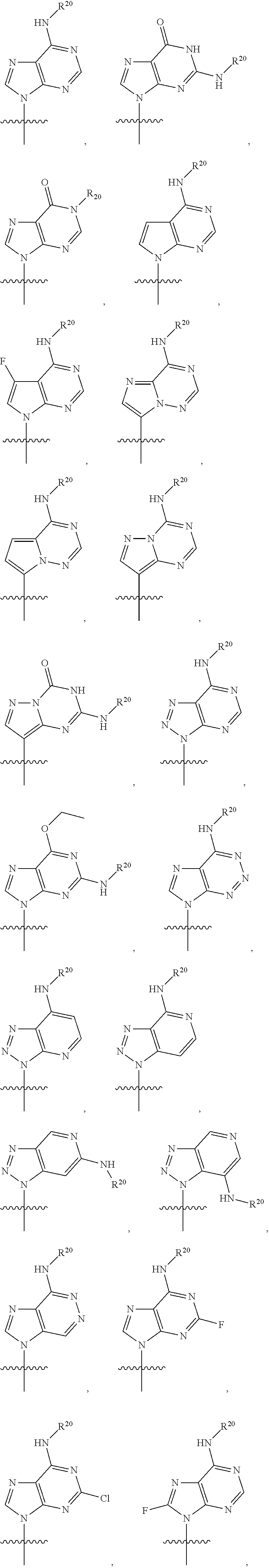

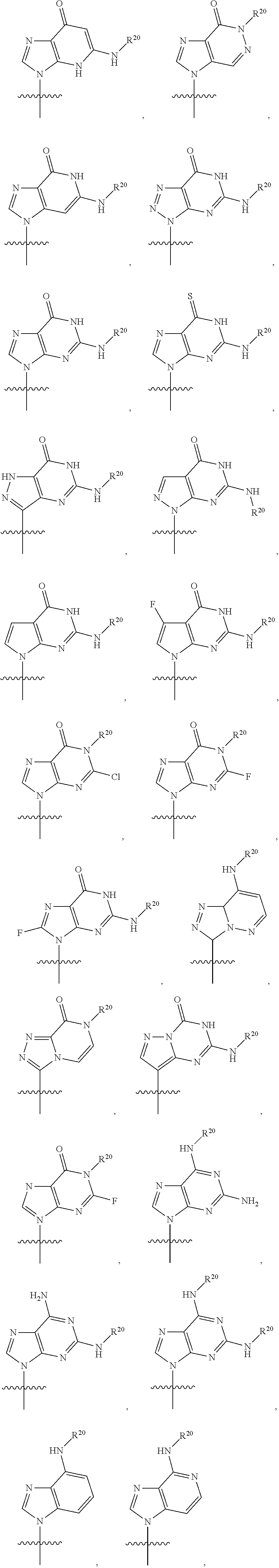

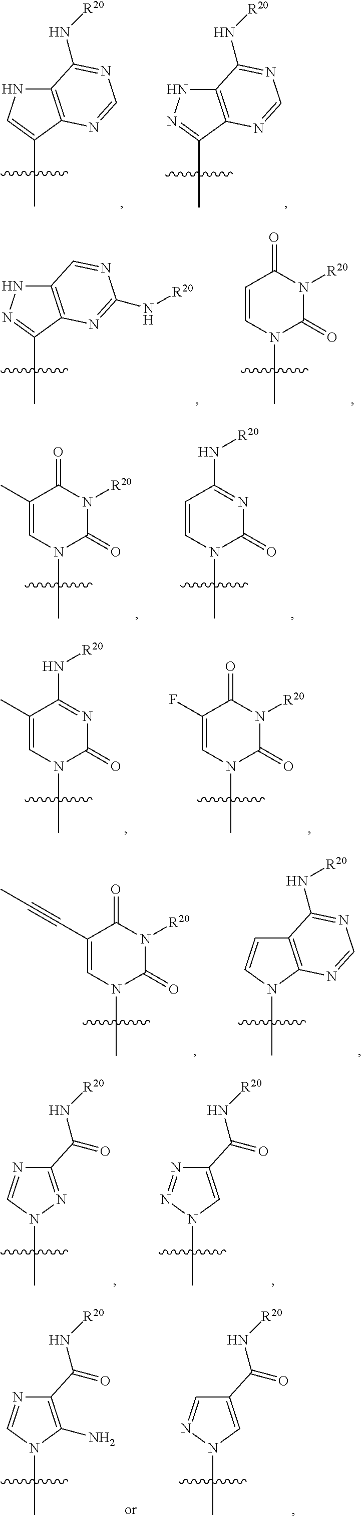

wherein: R¹ is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, O₃—C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —ON, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂, and
each R²⁰ is independently selected from H and L₁R¹⁵;
R^{1a} is

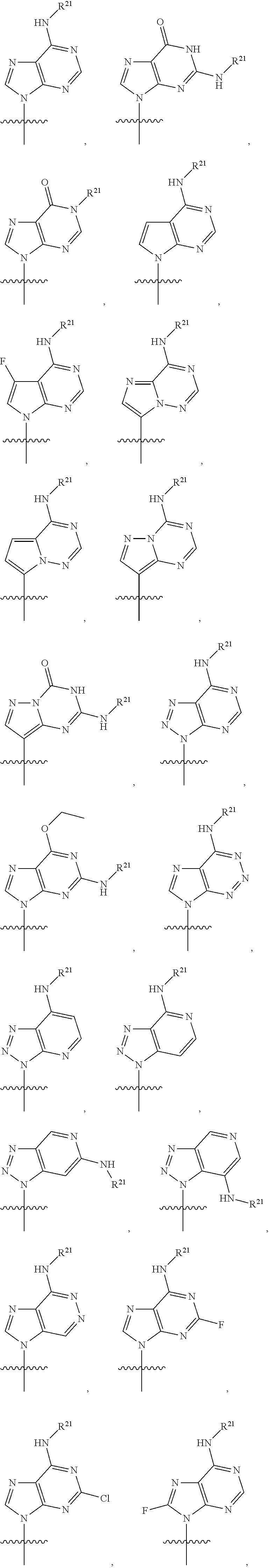

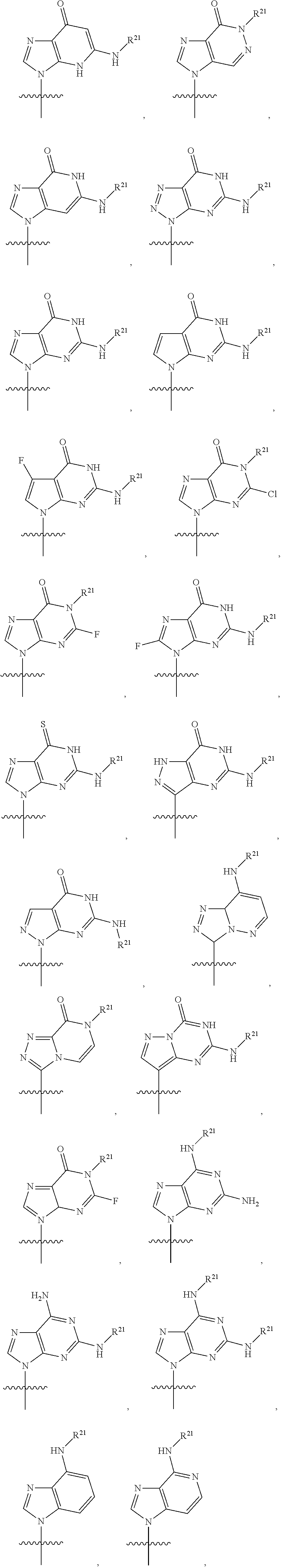

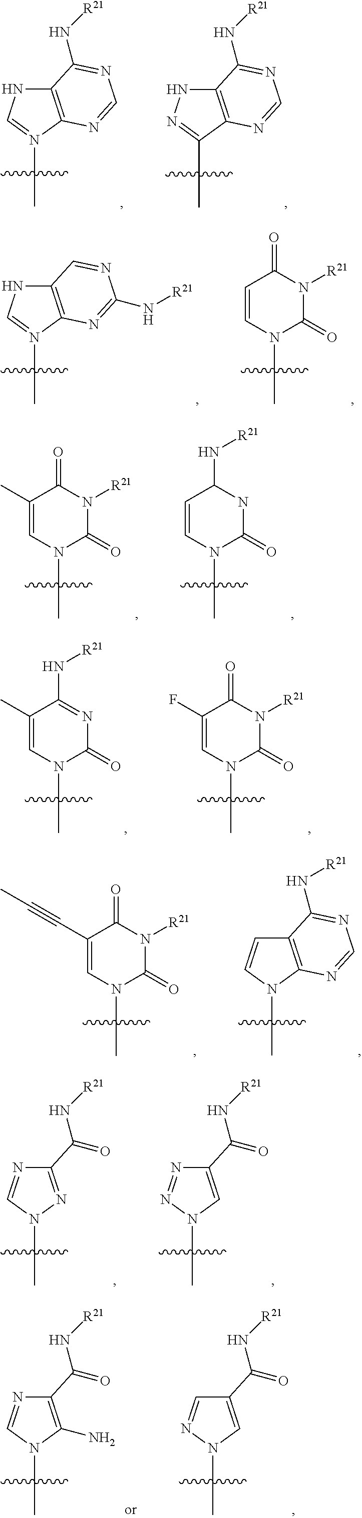

wherein: R^{1a} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²¹ is independently selected from H and L₁R¹⁵;
and
R¹ is

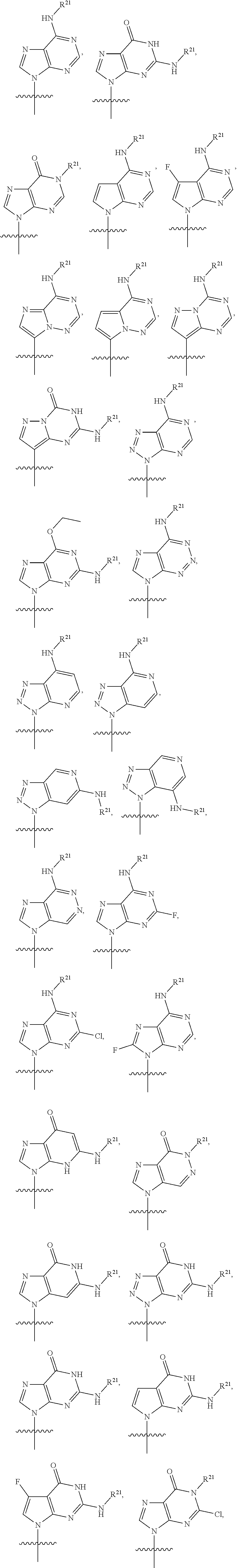

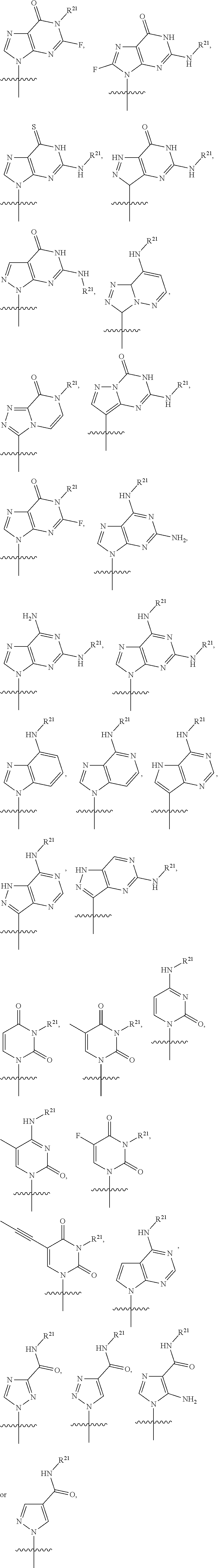

wherein: R^{1b} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²¹ is independently selected from H and L₁R¹⁵.

### Embodiment 94

A compound of Formula (A-3), Formula (B-3), Formula (C-3), Formula (D-3), Formula (E-3) or Formula (F-3), wherein:
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₁₁ is —O—, —S—, —S(═O)—, —SO₂—, —CH₂—, or —CF₂—;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₇ is O or S;
Y₈ is O or S;
R¹ is

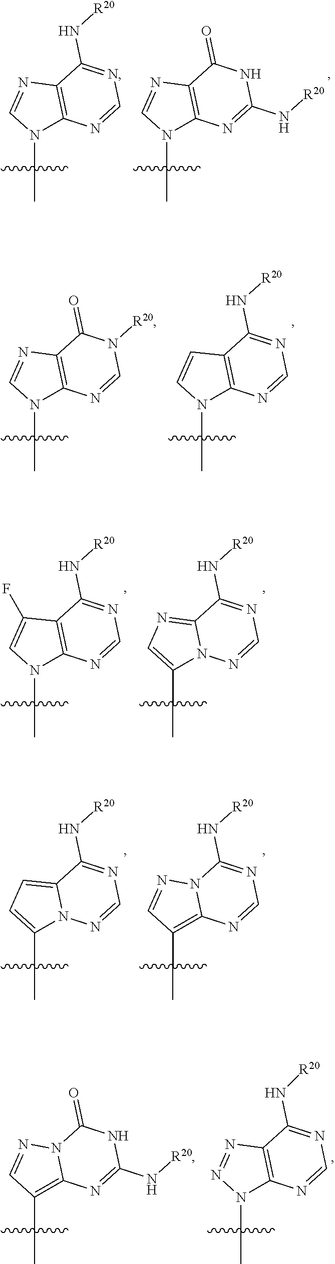

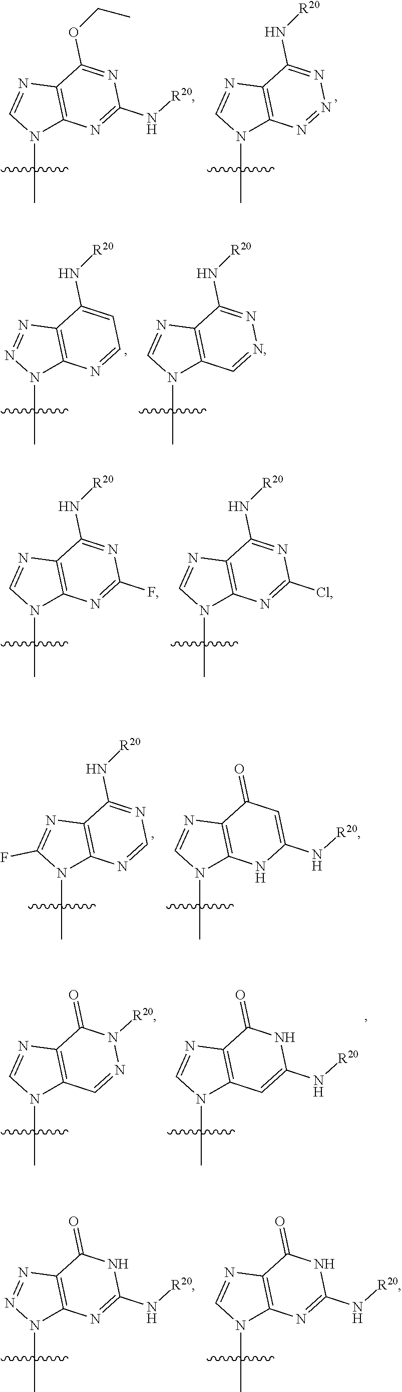

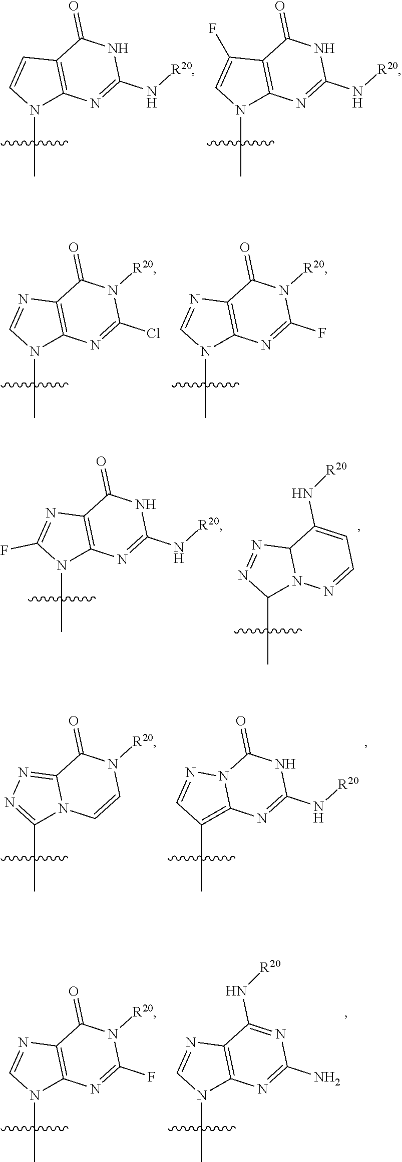

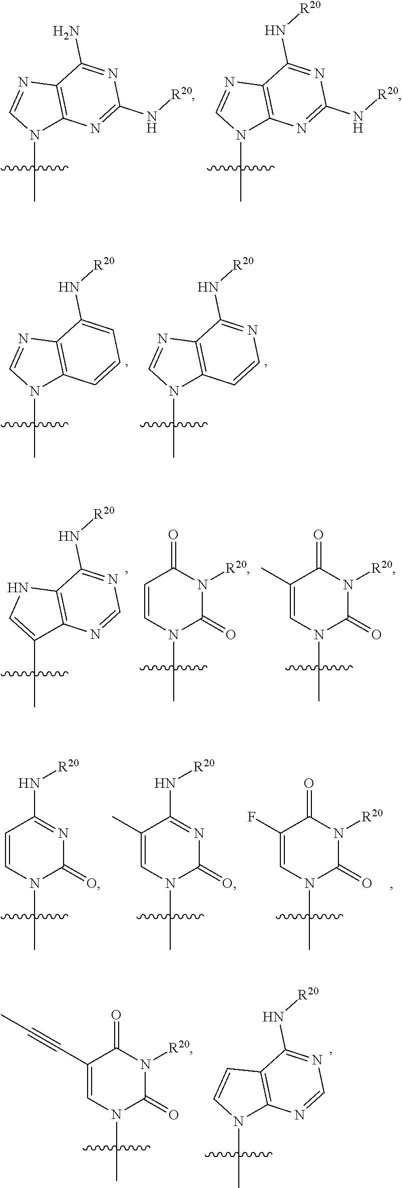

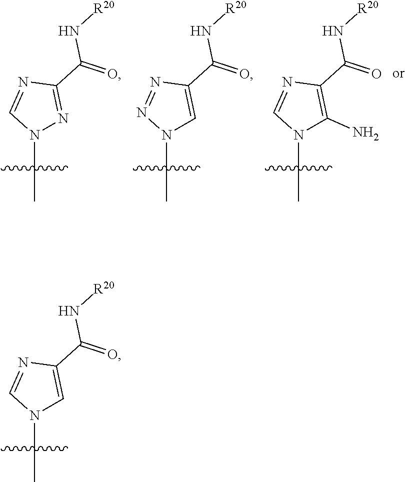

wherein: R¹ is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²⁰ is independently selected from H and L₁R¹⁵;
R^{1a} is

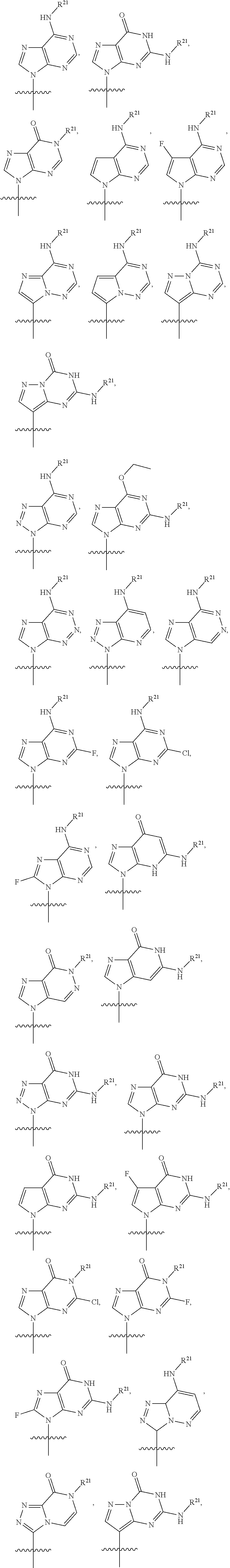

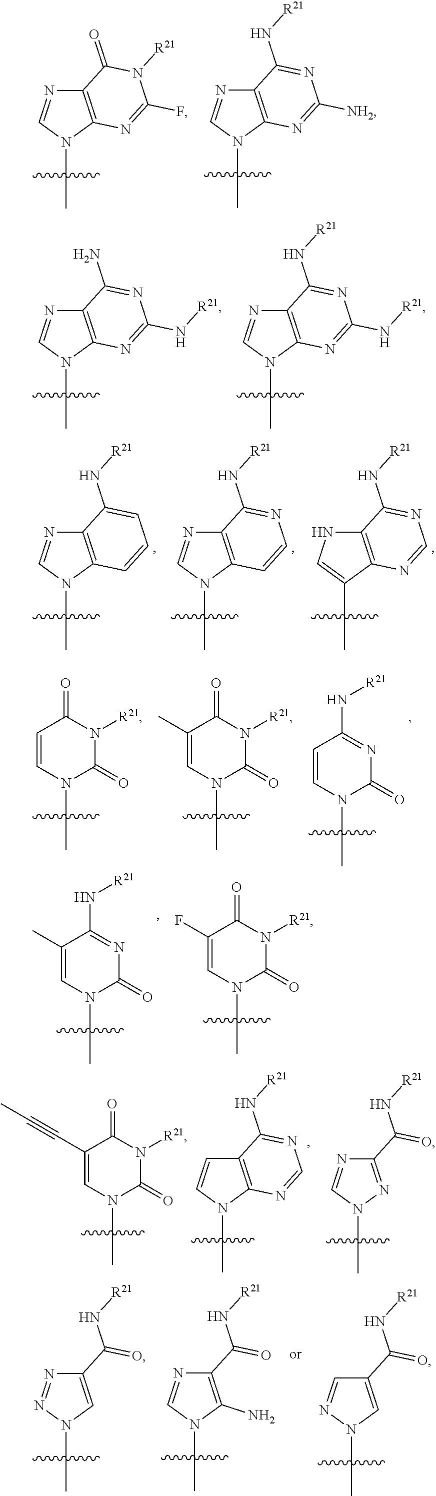

wherein: R^{1a} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²¹ is independently selected from H and L₁R¹⁵;
and
R^{1b} is

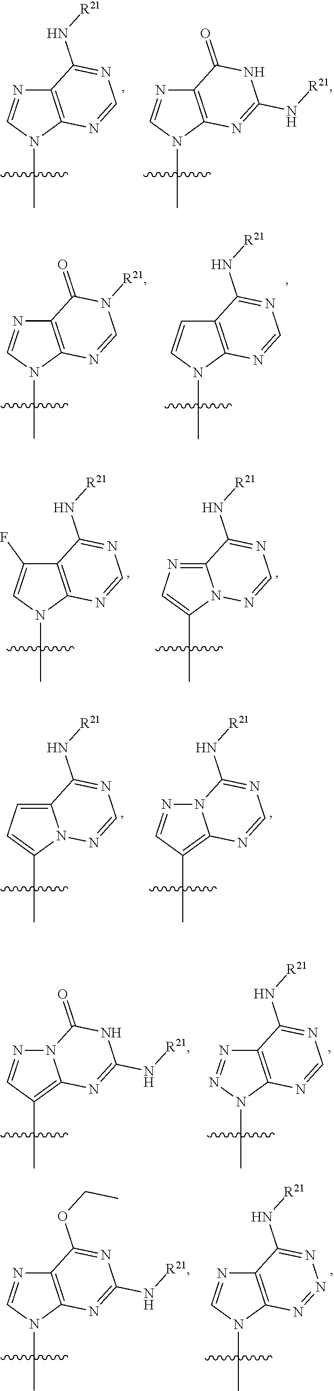

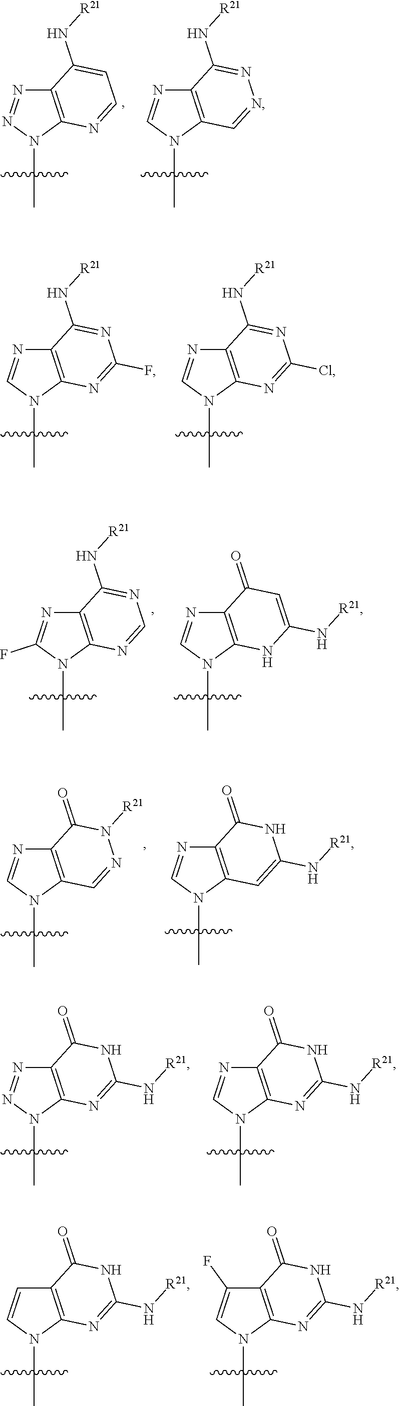

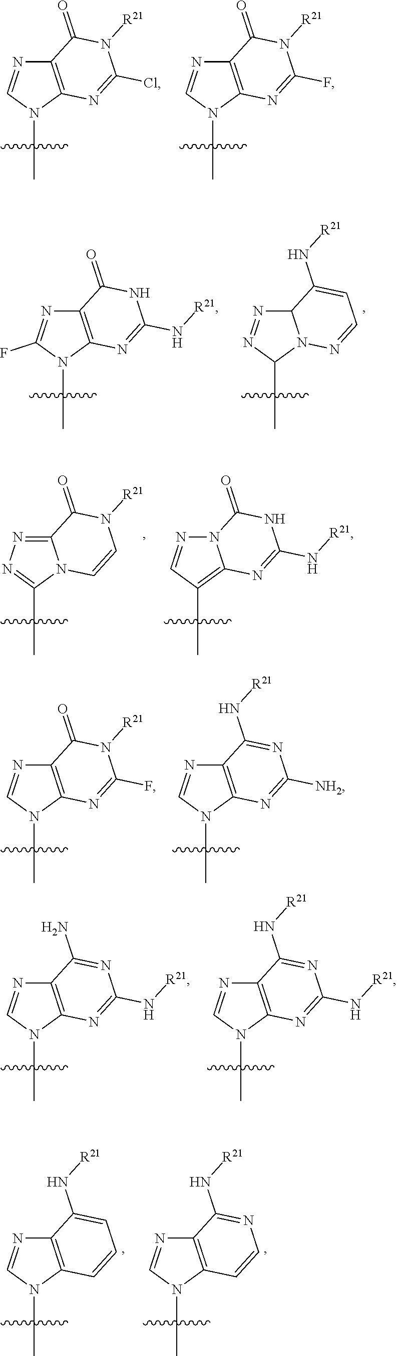

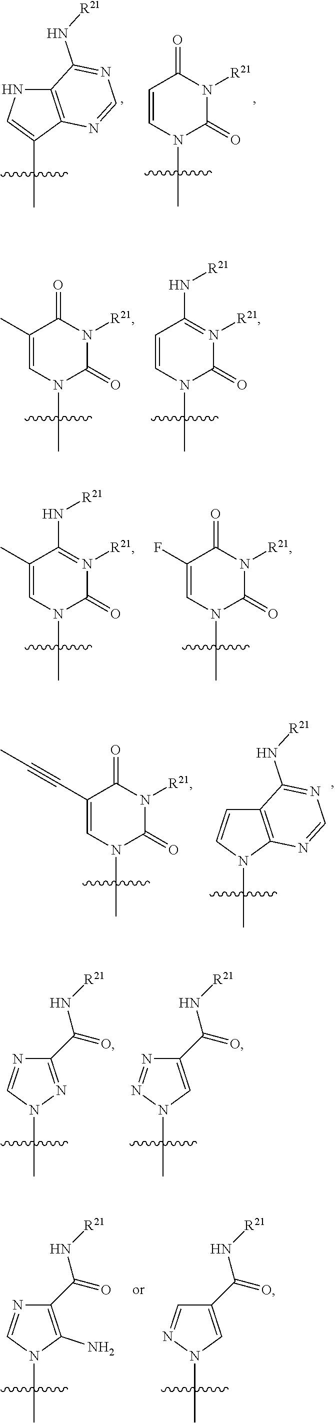

wherein: R^{1b} is substituted with 0, 1, 2 or 3 substituents independently selected from F, Cl, Br, OH, SH, NH₂, D, CD3, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²¹ is independently selected from H and L₁R¹⁵;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
R^{3a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
R^{4a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
R^{5a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
R^{7a} is selected from the group consisting of —OL₁R¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
each R¹⁰ is independently selected from the group consisting of H, C₁-C₁₂alkyl, —(CH₂CH₂O)ₙCH₂CH₂C(═O)OC₁-C₆alkyl, and

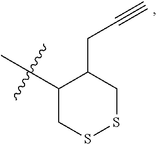 [SMILES: *C1CSSCC1CC#C]

wherein the C₁-C₁₂alkyl of R¹⁰ is substituted by 0, 1, 2 or 3 substituents independently selected from —OH, C₁-C₁₂alkoxy, —S—C(═O)C₁-C₆alkyl and C(O)OC₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position, and
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position;
L₁ is —C(═O)O(CH₂)ₘNR¹¹C(═O)(CH₂)ₘ—**; —C(═O)O(CH₂)ₘNR¹¹C(═O)(CH₂)ₘO(CH₂)ₘ—**; —C(═O)O(CH₂)ₘNR¹¹C(═O)X₁X₂C(═O)(CH₂)ₘ—**; —C(═O)OC(R¹²)₂(CH₂)ₘNR¹¹C(═O)X₁X₂C(═O)(CH₂)ₘ—**; —C(═O)O(CH₂)ₘNR¹¹C(═O)X₁X₂C(═O)X₂C)(CH₂)ₘO)(CH₂)ₘ—**; —C(═O)O(CH₂)ₘNR¹¹C(═O)X₁X₂C(═O)X₂C)(CH₂)ₘO)(CH₂)ₘC(═O)—**; —C(═O)O(CH₂)ₘNR¹¹C(═O)X₄C(═O)NR¹¹(CH₂)ₘNR¹¹C(═O)(CH₂)ₘO(CH₂)ₘ—**; —C(═O)O(CH₂)ₘNR¹¹C(═O)X₅C═O)X_{C}(═O)(CH₂)ₘNR¹¹C(═O)(CH₂)ₘ—**; —C(═O)X₄C(═O)NR(CH₂)ₘNR¹¹C(═O)(CH₂)ₘO(CH₂)ₘ—**; —C(═O)(CH₂)ₘNR¹¹C(═O)X₁X₂C(═O)(CH₂)ₘ—**; —C(═O)(CH₂)ₘNR¹¹C(═O)X₂C(═O)(CH₂)ₘ—**; —C(═O)O(CH₂)ₘX₆C(═O)X₁X₂C(═O) (CH₂)ₘ—**, —C(═O)(CH₂)ₘNR¹¹C(═O)((CH₂)ₘO)ₙ(CH₂)ₘ—**, —C(═O)O(CH₂)ₘX₆C(═O)(CH₂)ₘ—**, —C(═O)O(CH₂)ₘX₆C(═O)(CH₂)ₘO(CH₂)ₘ—**, —C(═O)O(CH₂)ₘX₆C(═O)X₁X₂C(═O) (CH₂)ₘ—**, —C(═O)O(CH₂)ₘX₆C(═O)X₁X₂C(═O) (CH₂)ₘO(CH₂)ₘ—**, —C(═O)O(CH₂)ₘX₆C(═O)X₁X₂C(═O)(CH₂)ₘO(CH₂)ₘC(═O)—**, —C(═O)O(CH₂)ₘX₆C(═O)X₄C(═O)NR¹¹(CH₂)ₘNR¹¹C(═O)(CH₂)ₘO(CH₂)ₘ—**, —C(═O)X₄C(═O)X₆(CH₂)ₘNR¹¹C(═O)(CH₂)ₘO(CH₂)ₘ—**, —C(═O)(CH₂)ₘX₆C(═O)X₁X₂C(═O)(CH₂)ₘ—**, —C(═O)O((CH₂)ₘO)ₙ(CH₂)ₘNR¹¹C(═O)X₅C(═O)(CH₂)ₘ—**; —C(═O)O((CH₂)ₘO)ₙ(CH₂)ₘNR¹¹C(═O)X₅C(═O)(CH₂)ₘNR¹¹C(═O)(CH₂)ₘ—**; —C(═O)O((CH₂)ₘO)ₙ(CH₂)ₘNR¹¹C(═O)X₅C(═O)(CH₂)ₘX₃(CH₂)ₘ—**; —C(═O)O((CH₂)ₘO)ₙ(CH₂)ₘNR¹¹C(═O)X₅C(═O)((CH₂)ₘO)ₙ(CH₂)ₘ—**; —C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘ—**; —C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)O((CH₂)ₘO)ₙ(CH₂)ₘNR¹¹C(═O))X₅C(═O)((CH₂)ₘO)ₙ(CH₂)ₘX₃(CH₂)ₘ—**; —C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘ—**; —C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O)O((CH₂)ₘO)ₙ(CH₂)ₘNR¹¹C(═O)X₅(CH₂)ₘX₃(CH₂)ₘ—**; —C(═O)O((CH₂)ₘO)ₙ(CH₂)ₘNR¹¹C(═O)X₅((CH₂)ₘO)ₙ(CH₂)ₘ—**; —C(═O)O((CH₂)ₘO)ₙ(CH₂)ₘNR¹¹C(═O)X₅((CH₂)ₘO)ₙ(CH₂)ₘNR¹¹C(═O)(CH₂) m-**; —C(═O)O((CH₂)ₘO)ₙ(CH₂)ₘNR¹¹C(═O)X₅((CH₂)ₘO)ₙ(CH₂)ₘNR¹¹C(═O)(CH₂)ₘX₃(CH₂)ₘ—**; —C(═O)O((CH₂)ₘO)ₙ(CH₂)ₘNR¹¹C(═O)X₅((CH₂)ₘO)ₙ(CH₂)ₘX₃(CH₂)ₘ—**; —C(═O)O((CH₂)ₘO)ₙ(CH₂)ₘNR¹¹C(═O)X₅(CH₂)ₘNR¹¹ ((CH₂)ₘO)ₙ(CH₂)ₘ—**; —C(═O)O((CH₂)ₘO)ₙ(CH₂)ₘNR¹¹C(═O)X₅C(═O)(CH₂)ₘNR¹¹ ((CH₂)ₘO)ₙ(CH₂)ₘX₃(CH₂)ₘ—**; —C(═O)O((CH₂)ₘO)ₙ(CH₂)ₘNR¹¹C(═O)X₅(CH₂)ₘ—**; —C(═O)O((CH₂)ₘO)ₙ(CH₂)ₘNR¹¹C(═O)X₅C(═O)((CH₂)ₘO)ₙ(CH₂)ₘ—**; —C(═O)O((CH₂)ₘO)ₙ(CH₂)ₘNR¹¹C(═O)X₅(CH₂)ₘX₃(CH₂)ₘ—**; C(═O)O(CH₂)ₘ—**; —C(═O)O((CH₂)ₘO)ₙ(CH₂)ₘ—**; C(═O)O(CH₂)ₘNR¹¹(CH₂)ₘ—**; —C(═O)O(CH₂)ₘNR¹¹ (CH₂)ₘC(═O)X₂X₁C(═O)—**; —C(═O)O(CH₂)ₘX₃(CH₂)ₘ—**; C(═O)O(H₂)ₘX₆C(═O)X₁X₂O(═O)((CH₂)ₘO)ₙ(CH₂)ₘ**; —C(═O)O((CH₂)ₘO)ₙ(CH₂)ₘX₃(CH₂)ₘ—**; —C(═O)O((CH₂)ₘO)ₙ(CH₂)ₘNR¹¹C(═O)(CH₂)ₘ—**; —C(═O)O(CH₂)ₘNR¹¹C(═O(CH₂)ₘX₃(CH₂)ₘ—**; —C(═O)O((CH₂)ₘO)ₙ(CH₂)ₘNR¹¹C(═O)(CH₂)ₘX₃(CH₂)ₘ—**; —C(═O)O((CH₂)ₘO)ₙX₃(CH₂)ₘ—**; C(═O)O((CH₂)ₘO)ₙ(CH₂)ₘX₃(CH₂)ₘ—**; —C(═O)O((CH₂)ₘO)ₙ(CH₂)ₘC(═O)NR¹¹(CH₂)ₘ—**; C(═O)O(CH₂)ₘC(R¹²)₂—**; —C(═O)OCH₂)ₘC(R¹²)₂SS(CH₂)ₘNR¹¹C(═O)(CH₂)ₘ—**; —C(═O)O(CH₂)ₘC(═O)NR¹¹(CH₂)ₘ—**; C(═O)(CH₂)ₘ—**; C(═O)((CH₂)ₘO)ₙ(CH₂)ₘ—**; —C(═O)(CH₂)ₘNR¹¹(CH₂)ₘ—**; C(═O)(CH₂)ₘNR¹¹(CH₂)ₘC(═O)X₂X_{C}(═O)**; —C(═O)(CH₂)ₘX₃(CH₂)ₘ—**; C(═O)((CH₂)ₘO)ₙ(CH₂)ₘX₃(CH₂)ₘ—**; —C(═O)(CH₂)ₘNR¹¹C(═O)(CH₂)ₘ—**; C(═O)((CH₂)ₘO)ₙ(CH₂)ₘNR¹¹C(═O)(CH₂)ₘ—**; —C(═O)(CH₂)ₘNR¹¹C(═O(CH₂)ₘX₃(CH₂)ₘ—**; (CH₂)ₘNR¹¹C(═O)X₁X₂C(═O)(CH₂)ₘ—**; —(CH₂)ₘ(CHOH)(CH₂)ₘNR¹¹C(═O)X₁X₂C(═O)(CH₂)ₘ**; —C(═O)((CH₂)ₘO)ₙ(CH₂)ₘNR¹¹C(═O)(CH₂)ₘX₃(CH₂)ₘ—**; C(═O)((CH₂)ₘO)ₙX₃(CH₂)ₘ**; —C(═O)((CH₂)ₘO)ₙ(CH₂)ₘX₃(CH₂)ₘ—**; C(═O)((CH₂)ₘO)ₙ(CH₂)ₘC(═O)NR¹¹ (CH₂)ₘ—**; —C(═O)(CH₂)ₘC(R¹²)₂—**; C(═O)((CH₂)ₘO)ₙ(CH₂)ₘNR¹¹C(═O)X₅C(═O)(CH₂)ₘ—**; —C(═O)((CH₂)ₘO)ₙ(CH₂)ₘNR¹¹C(═O)X₅C(═O)(CH₂)ₘNR¹¹C(═O)(CH₂)ₘ—**; —C(═O)((CH₂)ₘO)ₙ (CH₂)ₘNR¹¹C(═O)X₅C(═O)(CH₂)ₘX₃(CH₂)ₘ—**; —C(═O)((CH₂)ₘO)ₙ(CH₂)ₘNR¹¹C(═O)X₅C(═O)((CH₂)ₘO)ₙ(CH₂)ₘ—**; —C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘ—**; —C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)((CH₂)ₘO)ₙ(CH₂)ₘNR¹¹C(═O))X₅C(═O)((CH₂)ₘO)ₙ(CH₂)ₘX₃(CH₂)ₘ**; —C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘ—**; —C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘX₃(CH₂)ₘ—**; —C(═O)((CH₂)ₘO)ₙ(CH₂)ₘNR¹¹C(═O)X₅((CH₂)ₘO)ₙ(CH₂)ₘ—**; —C(═O)((CH₂)ₘO)ₙ(CH₂)ₘNR¹¹C(═O)X₅((CH₂)ₘO)ₙ(CH₂)ₘNR¹¹C(═O)(CH₂)ₘ—**; —C(═O)((CH₂)ₘO)ₙ(CH₂)ₘNR¹¹C(═O)X₅((CH₂)ₘO)ₙ(CH₂)ₘNR¹¹C(═O)(CH₂)ₘX₃(CH₂)ₘ—**; —C(═O)((CH₂)ₘO)ₙ(CH₂)ₘNR¹¹C(═O)X₅((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘNR¹¹((CH₂)ₘO)ₙ(CH₂)ₘ—**; —C(═O)((CH₂)ₘO)ₙ(CH₂)ₘNR¹¹C(═O)X₅C(═O)(CH₂)ₘNR¹¹((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘ—**; —C(═O)((CH₂)ₘO)ₙ(CH₂)ₘNR¹¹C(═O)X₅C(═O)((CH₂)ₘO)ₙ(CH₂)ₘ—**; —C(═O)((CH₂)ₘO)ₙ(CH₂)ₘNR¹¹C(═O)X₅(CH₂)ₘX₃(CH₂)ₘ—**. —C(═O)(CH₂)ₘC(R¹²)₂SS(CH₂)ₘNR¹¹C(═O)(CH₂)ₘ—**; C(═O)(CH₂)ₘC(═O)NR¹¹ (CH₂)ₘ—**; —C(═O)X₁X₂C(═O)(CH₂)ₘ—**; C(═O)X₁X₂C(═O)(CH₂)ₘNR¹¹C(═O)(CH₂)ₘ—**; —C(═O)X₁X₂C(═O)(CH₂)ₘX₃(CH₂)ₘ—**; C(═O)X₁X₂C(═O)((CH₂)ₘO)ₙ(CH₂)ₘ—**; —C(═O)X₁X₂C(═O)((CH₂)ₘO)ₙ(CH₂)ₘNR¹¹C(═O)(CH₂)ₘ—**; —C(═O)X₁X₂C(═O)((CH₂)ₘO)ₙ(CH₂)ₘNR¹¹C(═O) (CH₂)ₘX₃(CH₂)ₘ—**; —C(═O)X₁X₂C(═O)((CH₂)ₘO)ₙ(CH₂)ₘX₃(CH₂)ₘ—**; —C(═O)X₁X₂C(═O)(CH₂)ₘNR¹¹C(═O) ((CH₂)ₘO)ₙ(CH₂)ₘ—**; —C(═O)X₁X₂C(═O)(CH₂)ₘNR¹¹C(═O) ((CH₂)ₘO)ₙ(CH₂)ₘX₃(CH₂)ₘ—**; —C(═O)X₁X₂(CH₂)ₘX₃(CH₂)ₘ—**; C(═O)X₁X₂((CH₂)ₘO)ₙ(CH₂)ₘ—**; —C(═O)X₁X₂((CH₂)ₘO)ₙ(CH₂)ₘNR¹¹C(═O)(CH₂)ₘ—**; —C(═O)X₁X₂((CH₂)ₘO)ₙ(CH₂)ₘNR¹¹C(═O)(CH₂)ₘX₃(CH₂)ₘ—**; —C(═O)X₁X₂((CH₂)ₘO)ₙ(CH₂)ₘX₃(CH₂)ₘ—**; —C(═O)X₁X₂(CH₂)ₘNR¹¹ ((CH₂)ₘO)ₙ(CH₂)ₘ—**; —C(═O)X₁X₂C(═O)(CH₂)ₘNR¹¹((CH₂)ₘO)ₙ(CH₂)ₘX₃(CH₂)ₘ—**; —C(═O)NR¹¹(CH₂)ₘ—**; C(═O)NR¹¹(CH₂)ₘX₃(CH₂)ₘ—**; —C(═O)NR¹¹ (CH₂)ₘNR¹¹C(═O)X₁X₂C(═O)(CH₂)ₘ—**; —C(═O)NR¹¹ (CH₂)ₘNR¹¹C(═O)O(CH₂)ₘ—**; C(═O)NR¹¹(CH₂)ₘNR¹¹C(═O)X₁X₂—**; —C(═O)NR¹¹(CH₂)ₘNR¹¹C(═O)X₅; —C(═O)NR¹¹ (CH₂)ₘNR¹¹C(═O)(CH₂)ₘX₅(CH₂)ₘ**; —C(═O)X₁C(═O)NR¹¹ (CH₂)ₘX₅(CH₂)ₘ—**; —C(═O)X₆C(═O)(CH₂)ₘNR¹¹C(═O)X₁X₂C(═O)(CH₂)ₘ—** C(═O)NR¹¹(CH₂)ₘNR¹¹C(═O)(CH₂)ₘ—**; C(═O)NR¹¹(CH₂)ₘNR¹¹C(═O)(CH₂)ₘO(CH₂)ₘ—**; —C(═O)NR¹¹ (CH₂)ₘNR¹¹C(═O)X₁X₂C(═O)(CH₂)ₘO(CH₂)ₘ—**; —C(═O)NR¹¹ (CH₂)ₘNR¹¹C(═O)X₁X₂C(═O)(CH₂)ₘO(CH₂)ₘC(═O)—**; —C(═O)NR¹¹ (CH₂)ₘNR¹¹C(═O)X₄C(═O)NR¹¹ (CH₂)ₘNR¹¹C(═O)(CH₂)ₘO(CH₂)ₘ—**; —C(═O)NR¹¹(CH₂)ₘNR¹¹C(═O)X₁X₂C(═O)(CH₂)ₘ—**; —C(═O)NR¹¹ (CH₂)ₘNR¹¹C(═O)X₅C(═O)(CH₂)ₘ—**; —C(═O)NR¹¹ (CH₂)ₘNR¹¹C(═O)X₅C(═O)(CH₂)ₘNR¹¹C(═O)(CH₂)ₘ—**; —C(═O)NR¹¹ (CH₂)ₘNR¹¹C(═O)X₅C(═O)(CH₂)ₘX₃(CH₂)ₘ—**; —C(═O)NR¹¹ (CH₂)ₘNR¹¹C(═O)X₅C(═O)((CH₂)ₘO)ₙ(CH₂)ₘ—**; —C(═O)NR¹¹ (CH₂)ₘNR¹¹C(═O)X₅C(═O)((CH₂)ₘO)ₙ(CH₂)ₘNR¹¹C(═O)(CH₂)ₘ—**; —C(═O)NR¹¹ (CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)NR¹¹(CH₂)ₘNR¹¹C(═O)X₅C(═O)((CH₂)ₘO)ₙ(CH₂)ₘX₃(CH₂)ₘ—**; —C(═O)NR¹¹ (CH₂)ₘNR¹¹C(═O)X₅C(═O)(CH₂)ₘNR¹¹C(═O)((CH₂)ₘO)ₙ(CH₂)ₘ—**; —C(═O)NR¹¹ (CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O)NR¹¹ (CH₂)ₘNR¹¹C(═O)X₅(CH₂)ₘX₃(CH₂)ₘ—**; —C(═O)NR¹¹ (CH₂)ₘNR¹¹C(═O)X₅((CH₂)ₘO)ₙ(CH₂)ₘ—**; —C(═O)NR¹¹ (CH₂)ₘNR¹¹C(═O)X₅((CH₂)ₘO)ₙ(CH₂)ₘNR¹¹C(═O)(CH₂)ₘ—**; —C(═O)NR¹¹ (CH₂)ₘNR¹¹C(═O)X₅((CH₂)ₘO)ₙ(CH₂)ₘNR¹¹C(═O)(CH₂)ₘX₃(CH₂)ₘ—**; —C(═O)NR¹¹ (CH₂)ₘNR¹¹C(═O)X₅((CH₂)ₘO)ₙ(CH₂)ₘX₃(CH₂)ₘ—**; —C(═O)NR¹¹ (CH₂)ₘNR¹¹C(═O)X₅(CH₂)ₘNR¹¹((CH₂)ₘO)ₙ(CH₂)ₘ**; —C(═O)NR¹¹ (CH₂)ₘNR¹¹C(═O)X₅C(═O)(CH₂)ₘNR¹¹ ((CH₂)ₘO)ₙ(CH₂)ₘX₃(CH₂)ₘ—**; —C(═O)NR¹¹(CH₂)ₘNR¹¹C(═O)X₅(CH₂)ₘ—**; —C(═O)NR¹¹ (CH₂)ₘNR¹¹C(═O)X₅C(═O)((CH₂)ₘO)ₙ(CH₂)ₘ—**. —C(═O)NR¹¹(CH₂)ₘNR¹¹C(═O)X₅(CH₂)ₘX(CH₂)ₘ—**; —C(═O)X₁C(═O)NR¹¹ (CH₂)ₘNR¹¹C(═O)(CH₂)ₘ—**; —C(═O)X₁C(═O)NR¹¹(CH₂)ₘX(CH₂)ₘ—**; C(═O)NR¹¹(CH₂)ₘNR¹¹C(═O)(CH₂)ₘ—**; —C(═O)NR¹¹ (CH₂)ₘNR¹¹C(═O)(CH₂)ₘX₃(CH₂)ₘ—**; C(═O)NR¹¹(CH₂)ₘNR¹¹C(═O)—**; —C(═O)X₁X₂(CH₂)ₘ—**; C(═O)X₁X₂C(═O)((CH₂)ₘO)ₙ(CH₂)ₘ—**; —C(═O)X₁X₂(CH₂)ₘX₃(CH₂)ₘ—**; C(═O)NR¹¹ (CH₂)ₘX₃(CH₂)ₘ—**; —C(═O)NR¹¹ ((CH₂)ₘO)ₙ(CH₂)ₘX₃(CH₂)ₘ—**; C(═O)X₁X₂C(═O)((CH₂)ₘO)ₙ(CH₂)ₘ—**; —C(═O)X₁X₂C(═O)(CH₂)ₘ—**; —C(═O)X₁C(═O)(CH₂)ₘNR¹¹C(═O)(CH₂)ₘ—**; and —C(═O)X₁C(═O)(CH₂)ₘNR¹¹C(═O)((CH₂)ₘO)ₙ(CH₂)ₘ**;
   where the ** of L, indicates the point of attachment to R¹⁵;
R¹⁵ is

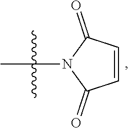 [SMILES: *N1C(=O)C=CC1=O]

—ONH₂, —NH₂,

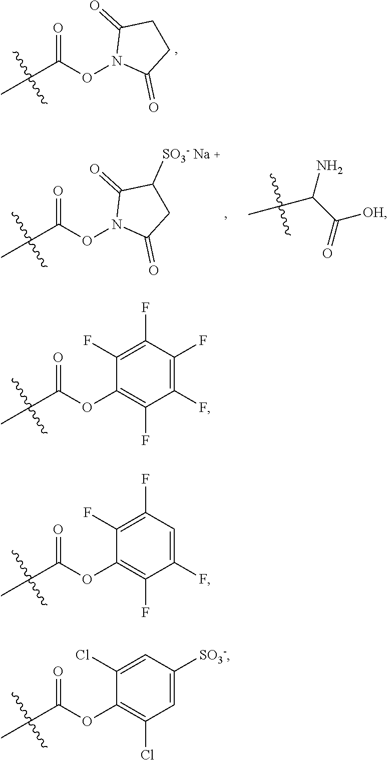

### —N₃,

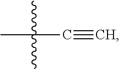 [SMILES: *C#C]

—SH, —SR¹², —SSR¹⁷, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I,

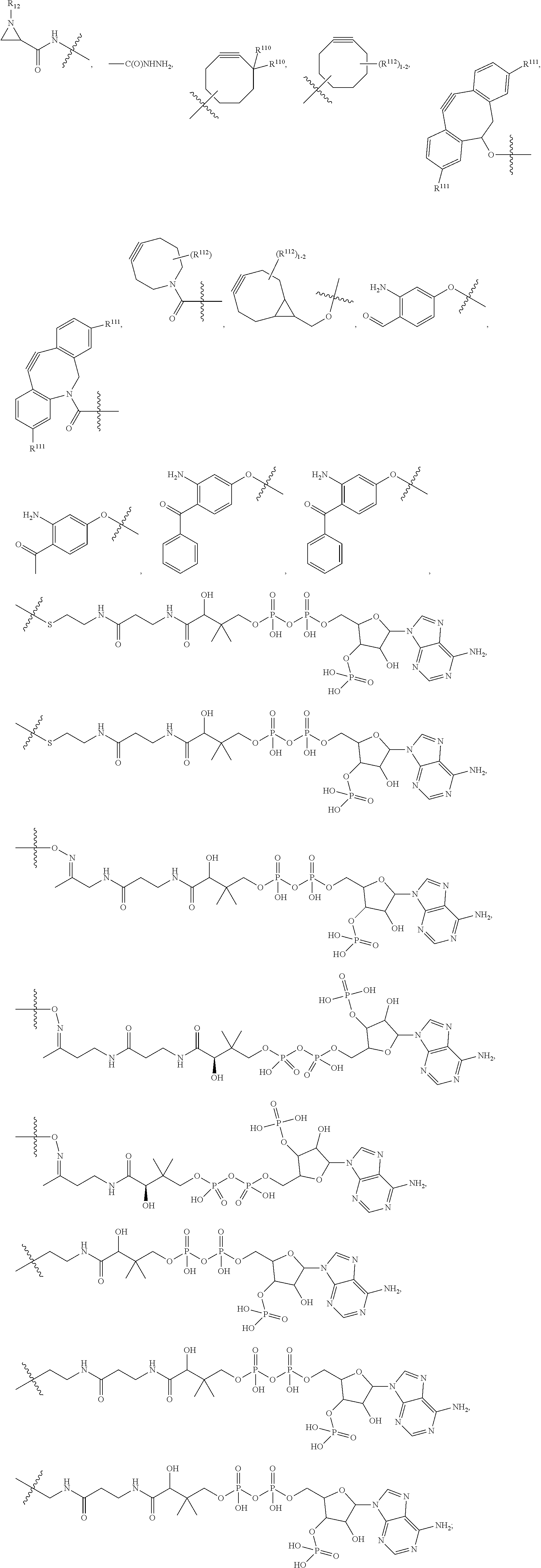

X₁ is

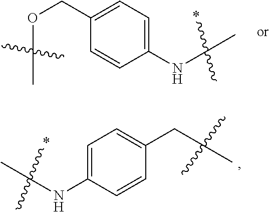 [SMILES: *Nc1ccc(CO*)cc1 ; *Cc1ccc(N*)cc1]

where the * of X₁ indicates the point of attachment to X₂;
X₂ is selected from

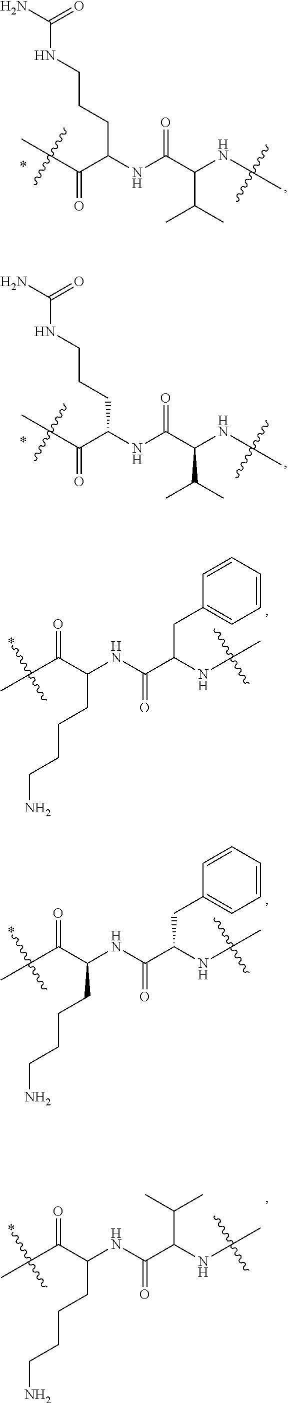 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O ; *NC(C(=O)NC(CCCCN)C(*)=O)C(C)C]

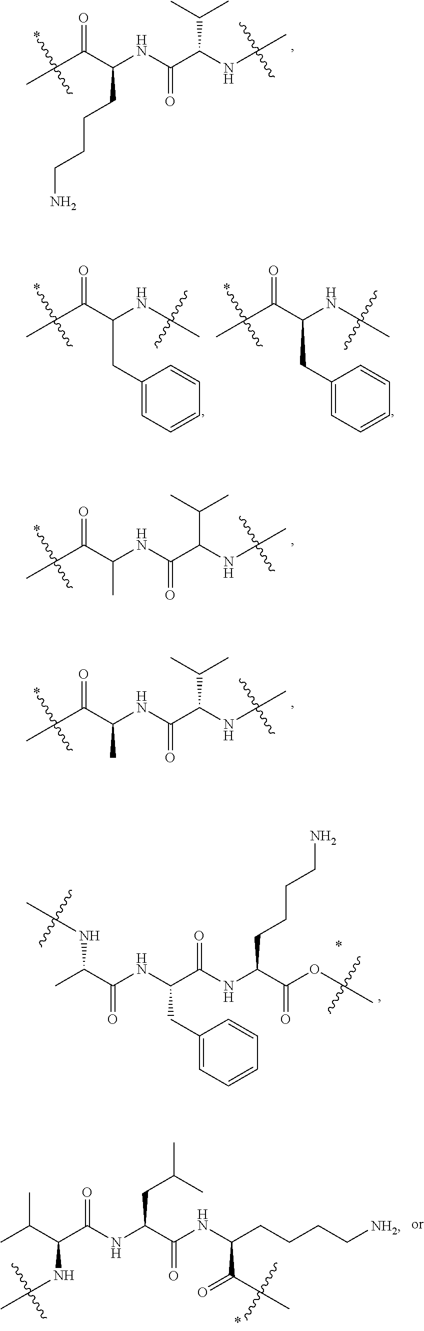

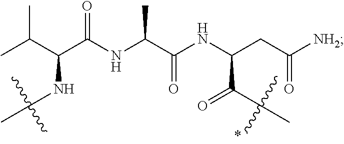 [SMILES: *N[C@H](C(=O)N[C@@H](C)C(=O)N[C@@H](CC(N)=O)C(*)=O)C(C)C]

where the * of X₂ indicates the point of attachment to X₁;
X₃ is

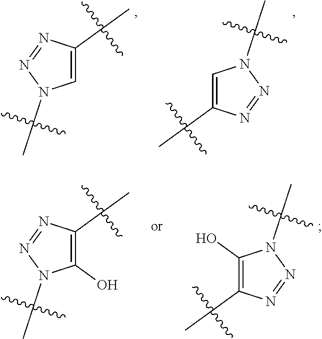 [SMILES: *c1cn(*)nn1 ; *c1cn(*)nn1 ; *c1nnn(*)c1O ; *c1nnn(*)c1O]

X₄ is —O(CH₂)ₙSSC(R¹²)₂(CH₂)ₙ— or —(CH₂)ₙC(R¹²)₂SS(CH₂)ₙO—;
X₅ is

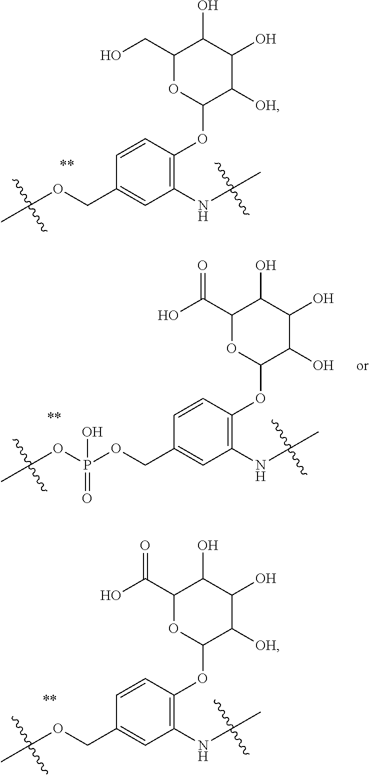 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the ** of X₅ indicates orientation toward R¹⁵;
X₆ is

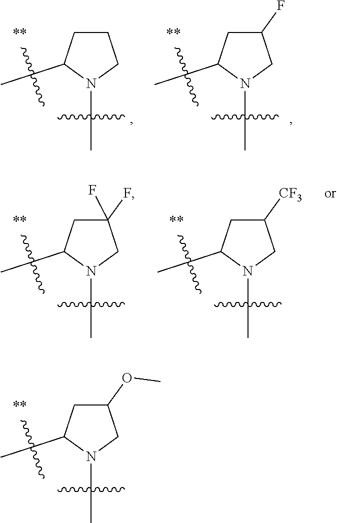

or, where the ** of X₆ indicates orientation toward R¹⁵;
R¹⁷ is 2-pyridyl or 4-pyridyl;
each R¹¹ is independently selected from H and C₁-C₆alkyl;
each R¹² is independently selected from H and C₁-C₆alkyl;
each m is independently selected from 1, 2, 3, 4, 5, 6, 7, 8, 9 and 10; and
each n is independently selected from 1, 2, 3, 4, 5, 6, 7, 8, 9, 10, 11, 12, 13, 14, 15, 16, 17 and 18.
each R¹¹⁰ is independently selected from H, C₁-C₆alkyl, F, Cl, and —OH;
each R¹¹¹ is independently selected from H, C₁-C₆alkyl, F, Cl, —NH₂, —OCH₃, —OCH₂CH₃, —N(CH₃)₂, —CN, —NO₂ and —OH;
each R¹¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
   and provided at least one of R²⁰ or R²¹ is —NHL₁R¹⁵ or is substituted with —NHL₁R¹⁵, or at least one of R³, R⁴, R⁵, R⁷, R^{3a}, R^{4a}, R^{5a} or R^{7a} is —OL₁R¹⁵.

### Embodiment 95

A compound of Formula (A-4), or a pharmaceutically acceptable salt thereof, wherein: R¹, R^{1a}, R³, R^{3a}, R⁶, R^{6a}, Y₃ and Y₄ are as defined in Embodiment 94.

### Embodiment 96

A compound of Formula (A-4a), Formula A-4b), Formula A-4c) or Formula A-4d), or a pharmaceutically acceptable salt thereof, wherein:
R¹, R^{1a}, R³, R^{3a}, R⁶ and R^{66a} are as defined in Embodiment 94;
Y₃ is OR⁹, N(R¹⁰)₂, SH or S⁻, and
Y₄ is OR⁹, N(R¹⁰)₂, SH or S⁻.

### Embodiment 97

A compound of Formula (A-4e), Formula (A-4f), Formula (A-4 g), Formula (A-4h), Formula (A-4i), Formula (A-4j), Formula (A-4k), Formula (A-41), Formula (A-4m), Formula (A-4n), Formula (A-4o) or Formula (A-4p), or a pharmaceutically acceptable salt thereof, wherein:
R¹, R^{1a}, R³, R^{3a}, R⁶ and R^{6a} are as defined in Embodiment 94;
Y₃ is OR⁹, N(R¹⁰)₂, SH or S⁻, and
Y₄ is OR⁹, N(R¹⁰)₂, SH or S⁻.

### Embodiment 98

A compound of Formula (B-4), or a pharmaceutically acceptable salt thereof, wherein: R¹, R^{1a}, R^{3a}, R⁵, R^{6a}, Y₃ and Y₄ are as defined in Embodiment 94.

### Embodiment 99

A compound of Formula (B-4a), Formula (B-4b), Formula (B-4c) or Formula (B-4d), or a pharmaceutically acceptable salt thereof, wherein:
R¹, R^{1a}, R^{3a}, R⁵ and R^{6a} are as defined in Embodiment 94;
Y₃ is OR⁹, N(R¹⁰)₂, SH or S⁻, and
Y₄ is OR⁹, N(R¹⁰)₂, SH or S⁻.

### Embodiment 100

A compound of Formula (B-4e), Formula (B-4f), Formula (B-4 g) or Formula (B-4h), or a pharmaceutically acceptable salt thereof, wherein:
R¹, R^{1a} and R⁵ are as defined in Embodiment 94;
Y₃ is OR¹⁰, N(R¹⁰)₂, SH or S⁻, and
Y₄ is OR¹⁰, N(R¹⁰)₂, SH or S⁻.

### Embodiment 101

A compound of Formula (C-4), or a pharmaceutically acceptable salt thereof, wherein: R¹, R^{1a}, R³, R^{5a}, R⁶, Y₃ and Y₄ are as defined in Embodiment 94.

### Embodiment 102

A compound of Formula (C-4a), Formula (C-4b), Formula (C-4c) or Formula (C-4d), or a pharmaceutically acceptable salt thereof, wherein:
R¹, R^{1a}, R³, R^{5a} and R⁶ are as defined in Embodiment 94;
Y₃ is OR¹⁰, N(R¹⁰)₂, SH or S⁻, and
Y₄ is OR¹⁰, N(R¹⁰)₂, SH or S⁻.

### Embodiment 103

A compound of Formula (C-4e), Formula (C-4f), Formula (C-4 g) or Formula (C-4h), or a pharmaceutically acceptable salt thereof, wherein:
R¹, R^{1a} and R^{5a} are as defined in Embodiment 94;
Y₃ is OR¹⁰, N(R¹⁰)₂, SH or S⁻, and
Y₄ is OR¹⁰, N(R¹⁰)₂, SH or S⁻.

### Embodiment 104

A compound of Formula (D-4), or a pharmaceutically acceptable salt thereof, wherein: R¹, R^{1a}, R⁵, R^{5a}, Y₃ and Y₄ are as defined in Embodiment 94.

### Embodiment 105

A compound of of Formula (D-4a), Formula (D-4b), Formula (D-4c) or Formula (D-4d), or a pharmaceutically acceptable salt thereof, wherein:
R¹, R^{1a}, R⁵ and R^{5a} are as defined in Embodiment 94;
Y₃ is OR¹⁰, N(R¹⁰)₂, SH or S⁻, and
Y₄ is OR¹⁰, N(R¹⁰)₂, SH or S⁻.

### Embodiment 106

A compound of Formula (E-4), or a pharmaceutically acceptable salt thereof, wherein: R¹, R^{1a}, R³, R^{3a}, R⁴, R^{4a}, R⁵ and R⁷ are as defined in Embodiment 94.

### Embodiment 107

A compound of Formula (E-4a) or Formula (E-4b), or a pharmaceutically acceptable salt thereof, wherein:
R¹, R³, R^{3a}, R⁴, R^{4a}, R⁵ and R⁷ are as defined in Embodiment 94;
and
Y₃ is OR¹⁰, N(R¹⁰)₂, SH or S⁻.

### Embodiment 108

A compound of Formula (F-4), or a pharmaceutically acceptable salt thereof, wherein: R¹, R^{1a}, R^{1b}, R³, R^{3a}, R⁴, R^{4a}, R⁵ and R⁷ are as defined in Embodiment 94.

### Embodiment 109

The compound of Formula (F-4a), Formula (F-4b), Formula (F-4c), or Formula (F-4d), or a pharmaceutically acceptable salt thereof, wherein:
R¹, R^{1a}, R^{1b}, R³, R^{3a}, R⁴, R^{4a}, R⁵ and R⁷ are as defined in Embodiment 94;
and
each Y₃ is independently selected from OR¹⁰, N(R¹⁰)₂, SH and S⁻.

### Embodiment 110

The compound of any one of Embodiments 76 to 109, wherein R¹ is

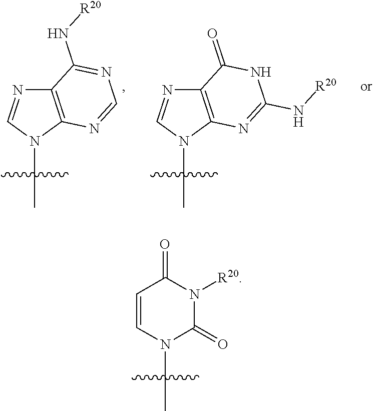

### Embodiment 111

The compound of any one of Embodiments 76 to 109, wherein R^{1a} is

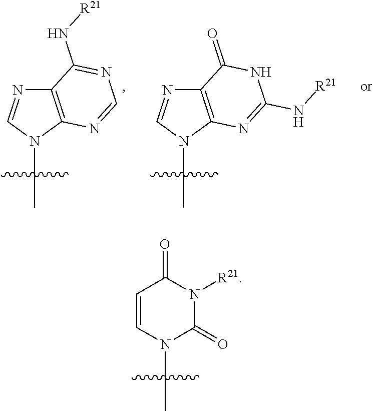

### Embodiment 112

The compound of any one of Embodiments 76 to 109, wherein R^{1b} is

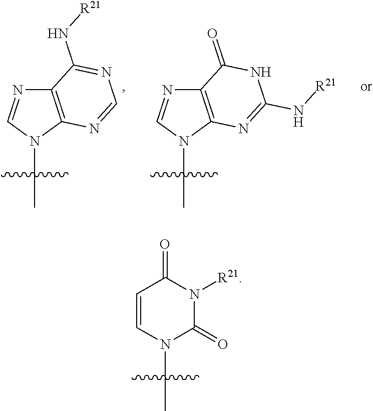

### Embodiment 113

The compound of any one of Embodiments 76 to 109, wherein R¹ is

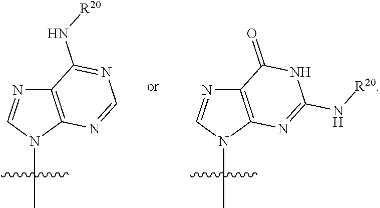

### Embodiment 114

The compound of any one of Embodiments 76 to 109, wherein R^{1a} is

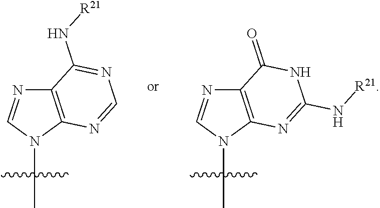

### Embodiment 115

The compound of any one of Embodiments 76 to 109, wherein R^{1b} is

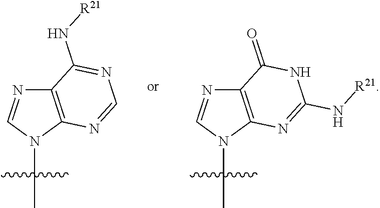

### Embodiment 116

The compound of any one of Embodiments 76 to 109, wherein R¹ is

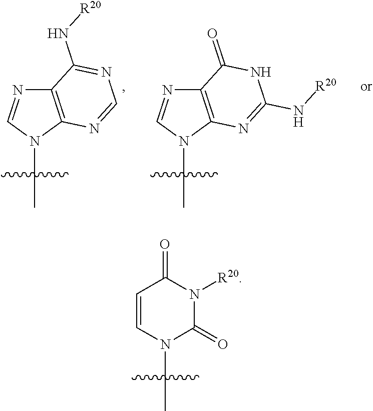

wherein R²⁰ is -L₁R¹⁵.

### Embodiment 117

The compound of any one of Embodiments 76 to 109, wherein R^{1a} is

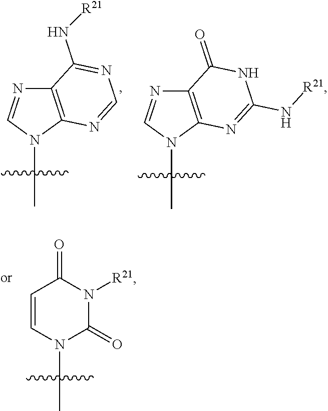

wherein R²¹ is -L₁R⁵.

### Embodiment 118

The compound of any one of Embodiments 76 to 109, wherein R^{1b} is

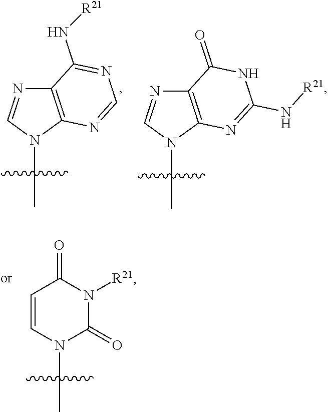

wherein R²¹ is -L₁R¹⁵

### Embodiment 119

The compound of any one of Embodiments 76 to 109, wherein R¹ is

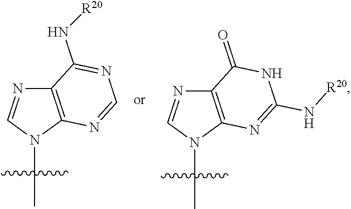

wherein R²⁰ is -L₁R¹⁵.

### Embodiment 120

The compound of any one of Embodiments 76 to 109, wherein R^{1a} is

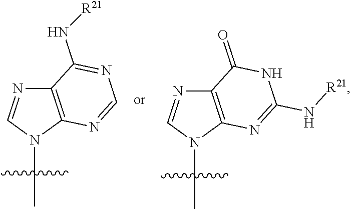

wherein R²¹ is -L₁R¹⁵.

### Embodiment 121

The compound of any one of Embodiments 76 to 109, wherein R^{1b} is

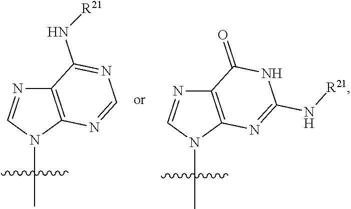

wherein R²¹ is -L₁R¹⁵.

### Embodiment 122

The compound of any one of Embodiments 76 to 109, wherein R¹ is

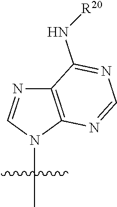

### and R^{1a} is

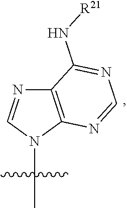

wherein R²⁰ is L₁R¹⁵ and R²¹ is H.

### Embodiment 123

The compound of any one of Embodiments 76 to 109, wherein R¹ is

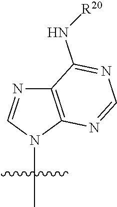

### and R^{1a} is

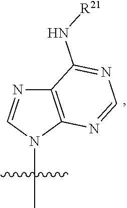

wherein R²⁰ is H and R²¹ is L₁R¹⁵.

### Embodiment 124

The compound of any one of Embodiments 76 to 109, wherein R¹ is

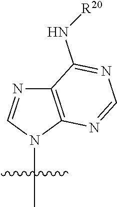

### and R^{1a} is

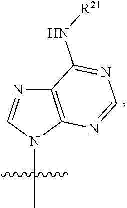

wherein R²⁰ is L₁R¹⁵ and R²¹ is L₁R¹⁵

### Embodiment 125

The compound of any one of Embodiments 76 to 109, wherein R¹ is

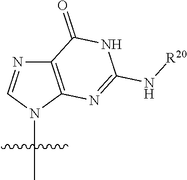

### and R^{1a} is

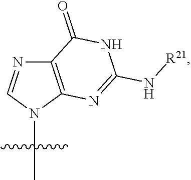

wherein R²⁰ is L₁R¹⁵ and R²¹ is H.

### Embodiment 126

The compound of any one of Embodiments 76 to 109, wherein R¹ is

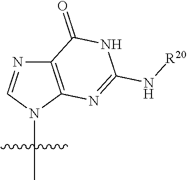

### and R^{1a} is

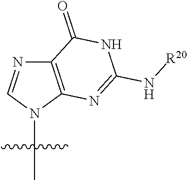

wherein R²⁰ is H and R²¹ is L₁R¹⁵.

### Embodiment 127

The compound of any one of Embodiments 76 to 109, wherein R¹ is

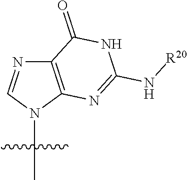

### and R^{1a} is

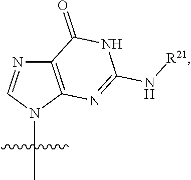

wherein R²⁰ is L₁R¹⁵ and R²¹ is L₁R¹⁵

### Embodiment 128

The compound of any one of Embodiments 76 to 109, wherein R¹ is

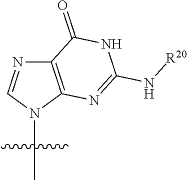

### and R^{1a} is

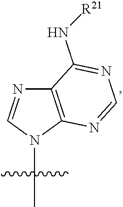

wherein R²⁰ is H and R²¹ is L₁R¹⁵

### Embodiment 129

The compound of any one of Embodiments 76 to 109, wherein R¹ is

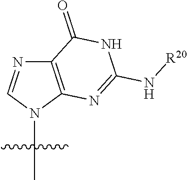

### and R^{1a} is

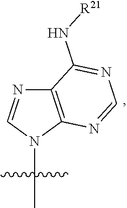

wherein R²⁰ is L₁R¹⁵ and R²¹ is H.

### Embodiment 130

The compound of any one of Embodiments 76 to 109, wherein R¹ is

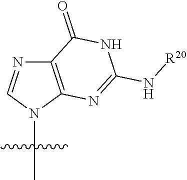

### and R^{1a} is

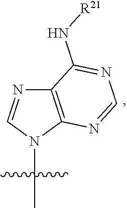

wherein R²⁰ is L₁R¹⁵ and R²¹ is L₁R¹⁵.

### Embodiment 131

The compound of any one of Embodiments 76 to 109, wherein R¹ is

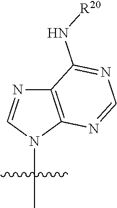

### and R^{1a} is

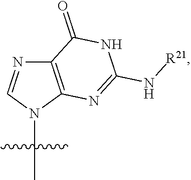

wherein R²⁰ is H and R²¹ is L₁R¹⁵.

### Embodiment 132

The compound of any one of Embodiments 76 to 109, wherein R¹ is

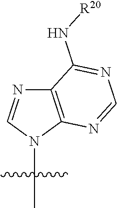

### and R^{1a} is

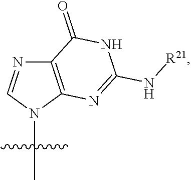

wherein R²⁰ is L₁R⁵ and R²¹ is H.

### Embodiment 133

The compound of any one of Embodiments 76 to 109, wherein R¹ is

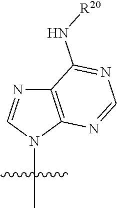

### and R^{1a} is

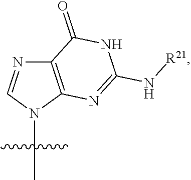

wherein R²⁰ is L₁R¹⁵ and R²¹ is L₁R¹⁵

### Embodiment 134

The compound of any one of Embodiments 76 to 109, wherein R¹ is

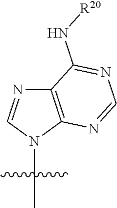

### and R^{1a} is

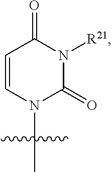

wherein R²⁰ is L₁R¹⁵ and R²¹ is H.

### Embodiment 135

The compound of any one of Embodiments 76 to 109, wherein R¹ is

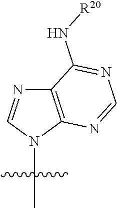

### and R^{1a} is

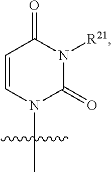

wherein R²⁰ is H and R²¹ is L₁R¹⁵.

### Embodiment 136

The compound of any one of Embodiments 76 to 109, wherein R¹ is

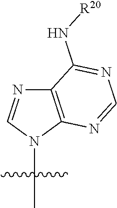

### and R^{1a} is

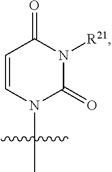

wherein R²⁰ is L₁R¹⁵ and R²¹ is L₁R¹⁵

### Embodiment 137

The compound of any one of Embodiments 76 to 109, wherein R¹ is

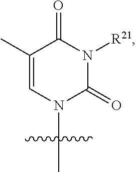

### R^{1b} is

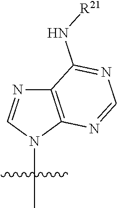

### and R^{1a} is

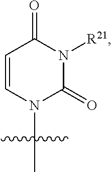

wherein R²⁰ is L₁R⁵ and each R²¹ is H.

### Embodiment 138

The compound of any one of Embodiments 76 to 109, wherein R¹ is

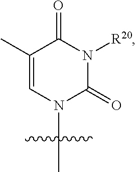

### R^{1b} is

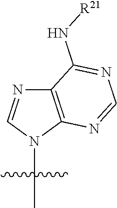

### and R^{1a} is

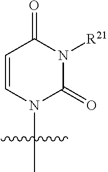

wherein R²⁰ is H, R²¹ of Rib is L₁R¹⁵ and R²¹ of R^{1a} is H.

### Embodiment 139

The compound of any one of Embodiments 76 to 109, wherein R¹ is

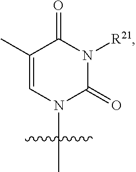

### R^{1b} is

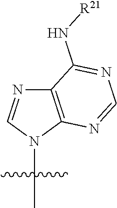

### and R^{1a} is

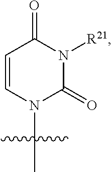

wherein R²⁰ is H, R²¹ of Rib is H and R²¹ of R^{1a} is L₁R¹⁵.

### Embodiment 140

The compound of any one of Embodiments 76 to 109, wherein R^{1a} is

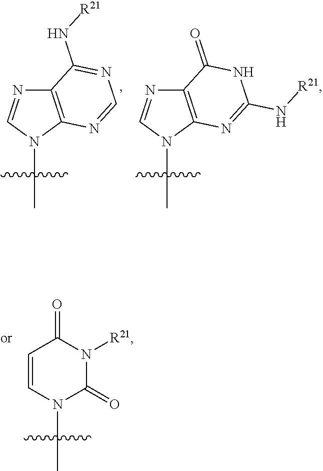

wherein R²¹ is H and one of R³, R^{3a}, R⁵ or R^{5a} is —OL₁R¹⁵.

### Embodiment 141

The compound of any one of Embodiments 76 to 109, wherein R^{1b} is

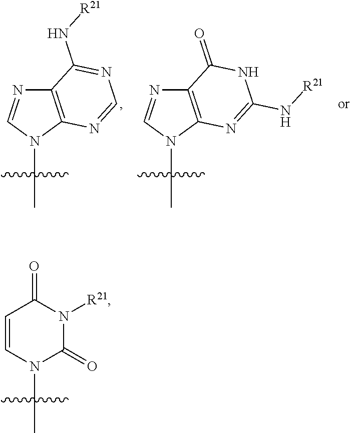

wherein R²¹ is H and one of R³, R^{3a}, R⁵ or R^{5a} is —OL₁R¹⁵.

### Embodiment 142

The compound of any one of Embodiments 76 to 109, wherein R¹ is

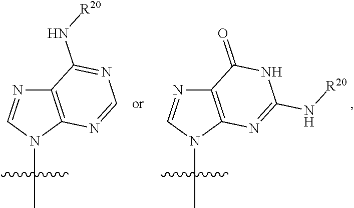

wherein R²⁰ is H and one of R³, R^{3a}, R⁵ or R^{5a} is —OL₁R¹⁵.

### Embodiment 143

The compound of any one of Embodiments 76 to 109, wherein R^{1a} is

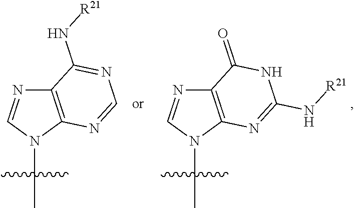

wherein R²¹ is H and one of R³, R^{3a}, R⁵ or R^{5a} is —OL₁R¹⁵.

### Embodiment 144

The compound of any one of Embodiments 76 to 109, wherein R^{1b} is

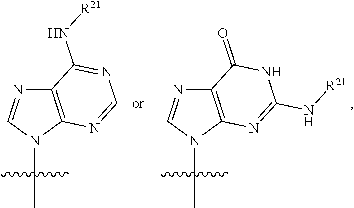

wherein R²¹ is H and one of R³, R^{3a}, R⁵ or R^{5a} is —OL₁R¹⁵.

### Embodiment 145

The compound of any one of Embodiments 76 to 109, wherein R¹ is

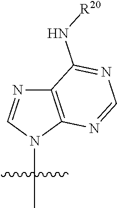

### and R^{1a} is

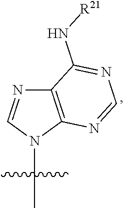

wherein R²⁰ is H, R²¹ is H and one of R³, R^{3a}, R⁵ or R^{5a} is —OL₁R¹⁵.

### Embodiment 146

The compound of any one of Embodiments 76 to 109, wherein R¹ is

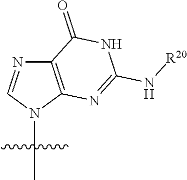

### and R^{1a} is

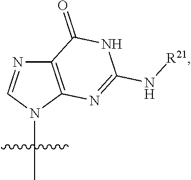

wherein R²⁰ is H, R²¹ is H and one of R³, R^{3a}, R⁵ or R^{5a} is —OL₁R¹⁵.

### Embodiment 147

The compound of any one of Embodiments 76 to 109, wherein R¹ is

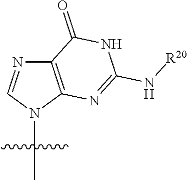

### and R^{1a} is

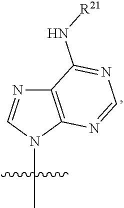

wherein R²⁰ is H, R²¹ is H and one of R³, R^{3a}, R⁵ or R^{5a} is —OL₁R¹⁵.

### Embodiment 148

The compound of any one of Embodiments 76 to 109, wherein R¹ is

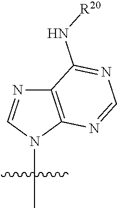

### and R^{1a} is

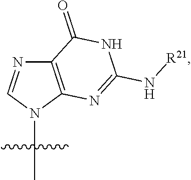

wherein R²⁰ is H, R²¹ is H and one of R³, R^{3a}, R⁵ or R^{5a} is —OL₁R¹⁵.

### Embodiment 149

The compound of any one of Embodiments 76 to 109, wherein R¹ is

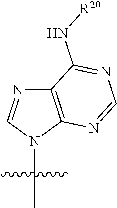

### and R¹ is

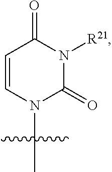

wherein R²⁰ is H, R²¹ is H and one of R³, R^{3a}, R⁵ or R^{5a} is —OL₁R¹⁵.

### Embodiment 150

The compound of any one of Embodiments 76 to 109, wherein R¹ is

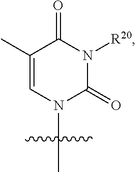

### R^{1b} is

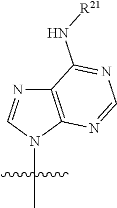

### and R^{1a} is

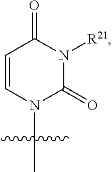

wherein R²⁰ is H, each R²¹ is H and one of R³, R^{3a}, R⁵ or R^{5a} is —OL₁R¹⁵.

### Embodiment 151

The compound of any one of Embodiments 76 to 150, wherein:
Y₃ is OH, O⁻, SH or S⁻, and
Y₄ is OH, O⁻, SH or S⁻.

### Embodiment 152

The compound of any one of Embodiments 76 to 150, wherein:
Y₃ is OH or O⁻, and
Y₄ is OH or O⁻.

### Embodiment 153

The compound of any one of Embodiments 76 to 150, wherein:
Y₃ is SH or S⁻, and
Y₄ is OH or O⁻.

### Embodiment 154

The compound of any one of Embodiments 76 to 150, wherein:
Y₃ is OH or O⁻, and
Y₄ is SH or S⁻.

### Embodiment 155

The compound of any one of Embodiments 76 to 150, wherein:
Y₃ is SH or S⁻, and
Y₄ is SH or S⁻.

### Embodiment 156

The compound of any one of Embodiments 76 to 139 or Embodiments 151 to 155, wherein: R², R^{2a}, R⁴, R^{4a}, R⁶, R^{6a}, R⁷ and R^{7a} are each H.

### Embodiment 157

The compound of any one of Embodiments 76 to 139 or Embodiments 151 to 155, wherein: R³ is —OH, F or —NH₂.

### Embodiment 158

The compound of any one of Embodiments 76 to 139 or Embodiments 151 to 155, wherein: R³ is —OH or F.

### Embodiment 159

The compound of any one of Embodiments 76 to 139 or Embodiments 151 to 155, wherein: R^{3a} is —OH, F or —NH₂.

### Embodiment 160

The compound of any one of Embodiments 76 to 139 or Embodiments 151 to 155, wherein: R^{3a} is —OH or F.

### Embodiment 161

The compound of any one of Embodiments 76 to 139 or Embodiments 151 to 155, wherein: R⁵ is —OH, F or —NH₂.

### Embodiment 162

The compound of any one of Embodiments 76 to 139 or Embodiments 151 to 155, wherein: R⁵ is —OH or F.

### Embodiment 163

The compound of any one of Embodiments 76 to 139 or Embodiments 151 to 155, wherein: R^{5a} is —OH, F or —NH₂.

### Embodiment 164

The compound of any one of Embodiments 76 to 139 or Embodiments 151 to 155, wherein: R^{5a} is —OH or F.

### Embodiment 165

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is —OH, and
R^{3a} is F.

### Embodiment 166

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is F, and
R^{3a} is —OH.

### Embodiment 167

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is F, and
R^{3a} is F.

### Embodiment 168

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is —OH, and
R^{3a} is —OH.

### Embodiment 169

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R^{3a} is —OH, and
R⁵ is F.

### Embodiment 170

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R^{3a} is F, and
R⁵ is —OH.

### Embodiment 171

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R^{3a} is F, and
R⁵ is F.

### Embodiment 172

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R^{3a} is —OH, and
R⁵ is —OH.

### Embodiment 173

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is —OH, and
R^{5a} is F.

### Embodiment 174

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is F, and
R^{5a} is —OH.

### Embodiment 175

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is F, and
R^{5a} is F.

### Embodiment 176

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R³ is —OH, and
R^{5a} is —OH.

### Embodiment 177

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R⁵ is —OH, and
R^{5a} is F.

### Embodiment 178

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R⁵ is F, and
R^{5a} is —OH.

### Embodiment 179

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R⁵ is F, and
R^{5a} is F.

### Embodiment 180

The compound of any one of Embodiments 76 to 139 or Embodiments 151 to 155, wherein when present:
R², R^{2a}, R⁴, R^{4a}, R⁶, R^{6a}, R⁷ and R^{7a} are each H;
R⁵ is —OH, and
R^{5a} is —OH.

### Embodiment 181

The compound of any one of Embodiments 76 to 139 or Embodiments 151 to 155, wherein:
R³ is —OH or F;
R^{3a} is —OH or F;
R⁵ is —OH or F;
R^{5a} is —OH or F;
R⁶ is H, and
R^{6a} is H.

### Embodiment 182

The compound of any one of Embodiments 76 to 139 or Embodiments 151 to 155, wherein:
R³ is H, —OH or F;
R^{3a} is H, —OCH₃, —OH or F;
R⁵ is —OH or F;
R⁴, R^{4a}, R⁶, R^{6a}, R⁷, R^{7a} are H, and
R^{6a} is H.

### Embodiment 183

The compound of any one of Embodiments 76 to 182, wherein:
L₁ is —C(═O)O(CH₂)ₘNR¹¹C(═O)(CH₂)ₘ—**; —C(═O)O(CH₂)ₘNR¹¹C(═O)(CH₂)ₘO(CH₂)ₘ—**; —C(═O)O(CH₂)ₘNR¹¹C(═O)X₁X₂C(═O)(CH₂)ₘ—**; —C(═O)OC(R¹²)₂(CH₂)ₘNR¹¹C(═O)X₁X₂C(═O)(CH₂)ₘ—**; —C(═O)O(CH₂)ₘNR¹¹C(═O)X₂C═O)X₂C(═CH₂)O(CH₂)ₘ—**; —C(═O)O(CH₂)ₘNR¹¹C(═O)X₁X₂C(═O)(CH₂)ₘO(CH₂)ₘC(═O)—**; —C(═O)O(CH₂)ₘNR¹¹C(═O)X₄C(═O)NR¹¹ (CH₂)ₘNR¹¹C(═O)(CH₂)ₘO(CH₂)ₘ—**; —C(═O)O(CH₂)ₘNR¹¹C(═O)X₅C(═O)(CH₂)ₘNR¹¹C(═O)(CH₂)ₘ—**; —C(═O)O(CH₂)ₘX₆C(═O)X₁X₂C(═O)((CH₂)ₘO)ₙ(CH₂)ₘ**; —(CH₂)ₘNR¹¹C(═O)X₁X₂C(═O)(CH₂)ₘ—**; —(CH₂)ₘ(CHOH)(CH₂)ₘNR¹¹C(═O)X₁X₂C(═O)(CH₂)ₘ**; —C(═O)X₆C(═O)(CH₂)ₘNR¹¹C(═O)X₁X₂C(═O) (CH₂)ₘ—**; —C(═O)X₄C(═O)NR¹¹(CH₂)ₘNR¹¹C(═O)(CH₂)ₘO(CH₂)ₘ—**; —C(═O)(CH₂)ₘNR¹¹C(═O)X₁X₂C(═O)(CH₂)ₘ—**; —C(═O)O(CH₂)ₘX₆C(═O)X₁X₂C(═O)(CH₂)ₘ—**, or —C(═O)(CH₂)ₘNR¹¹C(═O)((CH₂)ₘO)ₙ(CH₂)ₘ—**,
where the ** of L₁ indicates the point of attachment to R¹⁵ and
where R¹¹, R¹², X₁, X₂, m and n are s defined in Embodiment 94.

### Embodiment 184

The compound of any one of Embodiments 76 to 183, wherein:
L₁ is

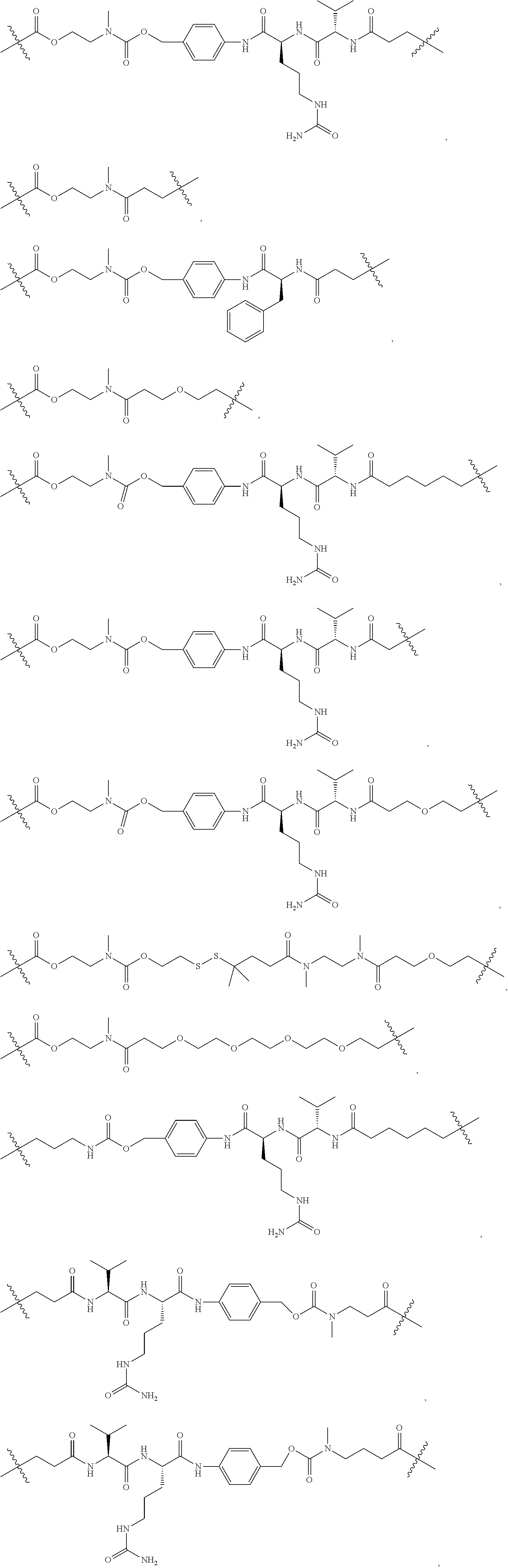

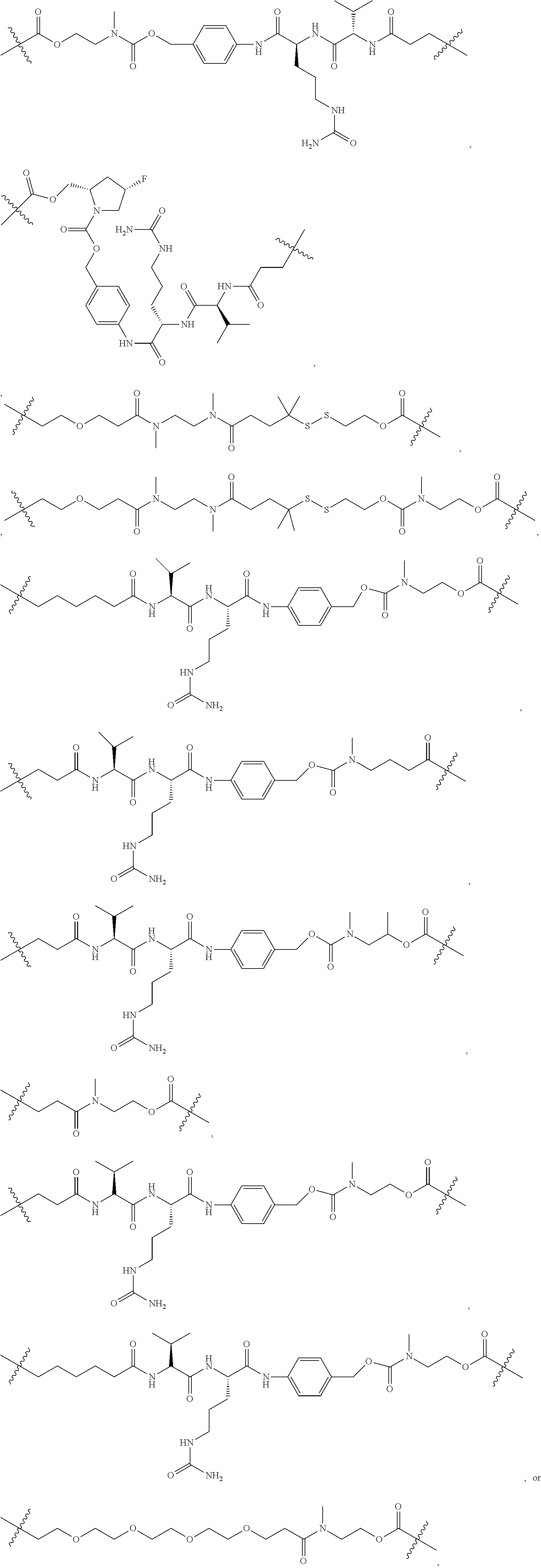

### Embodiment 185

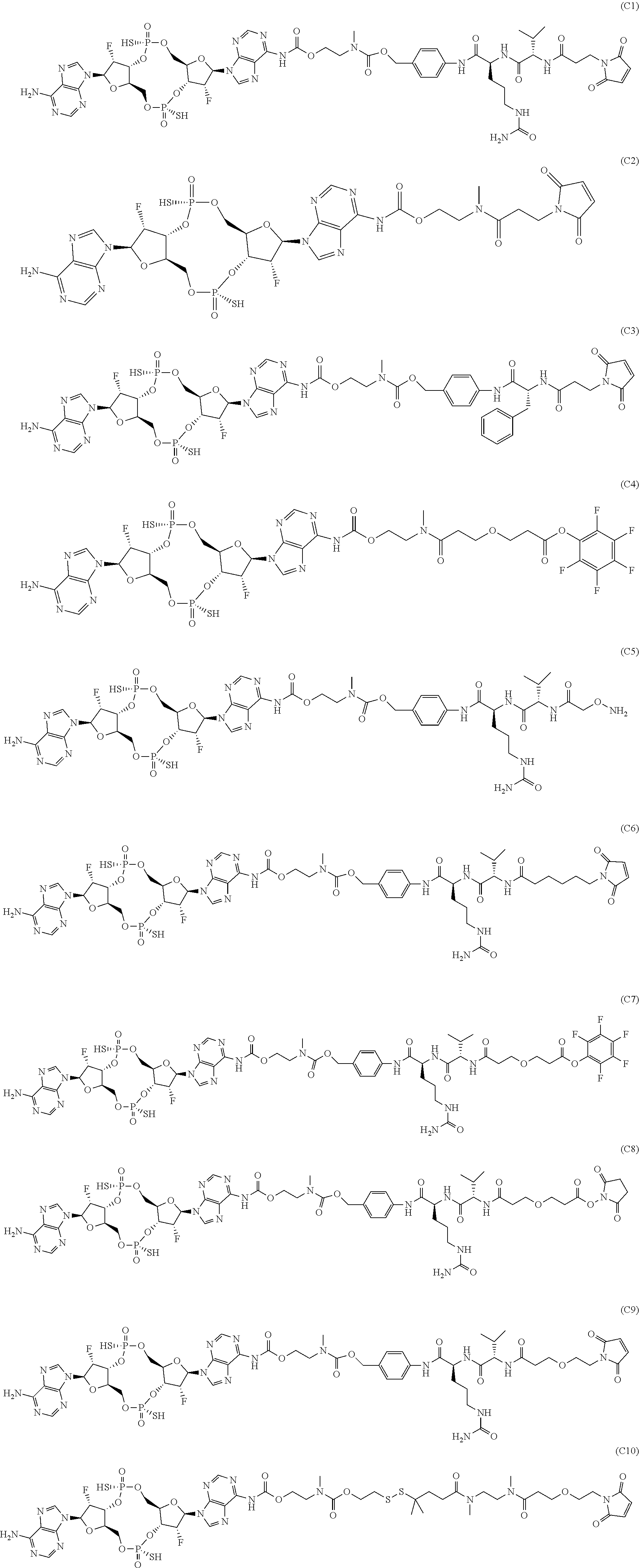

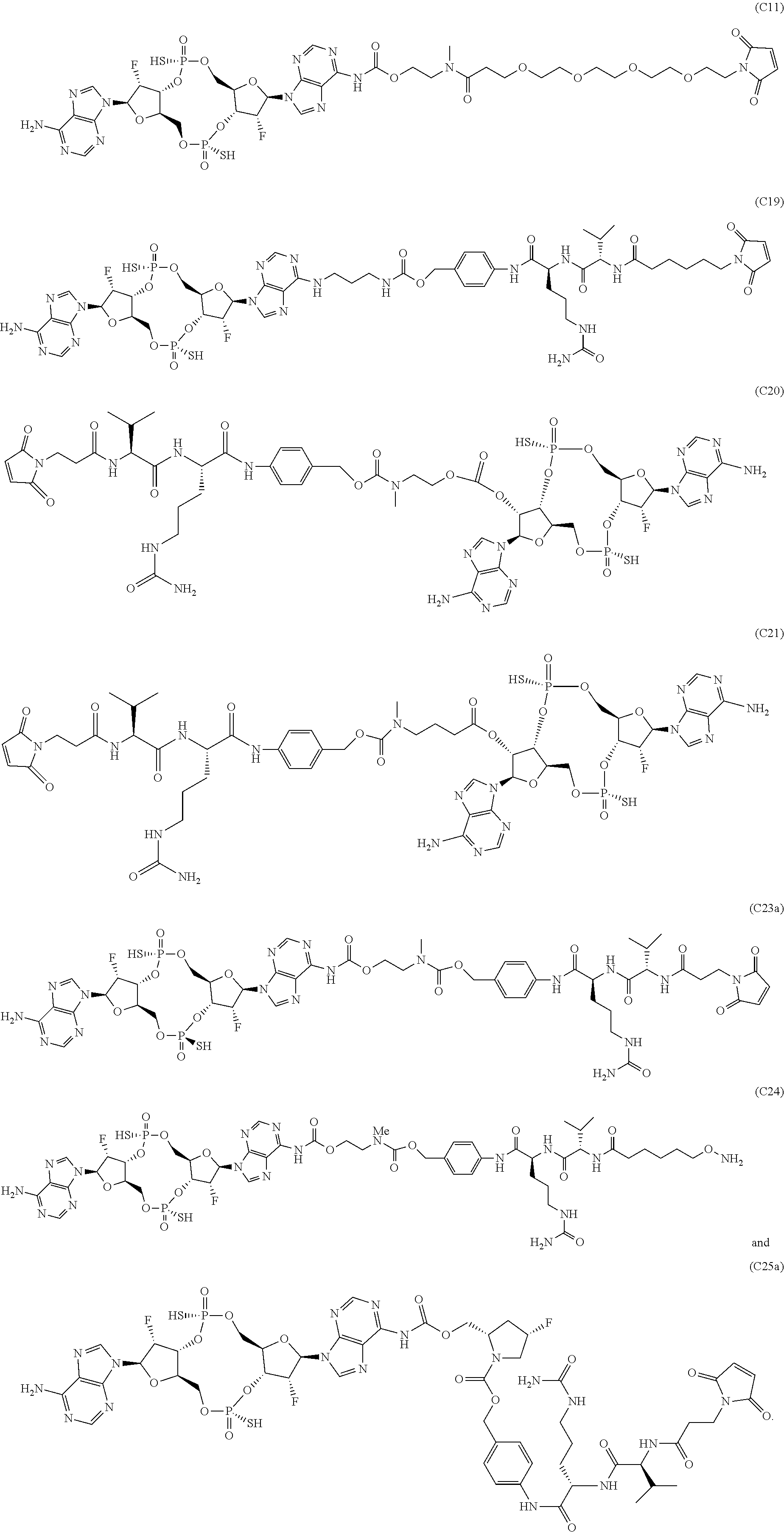

### Embodiment 186

A compound of Formula (A) selected from:

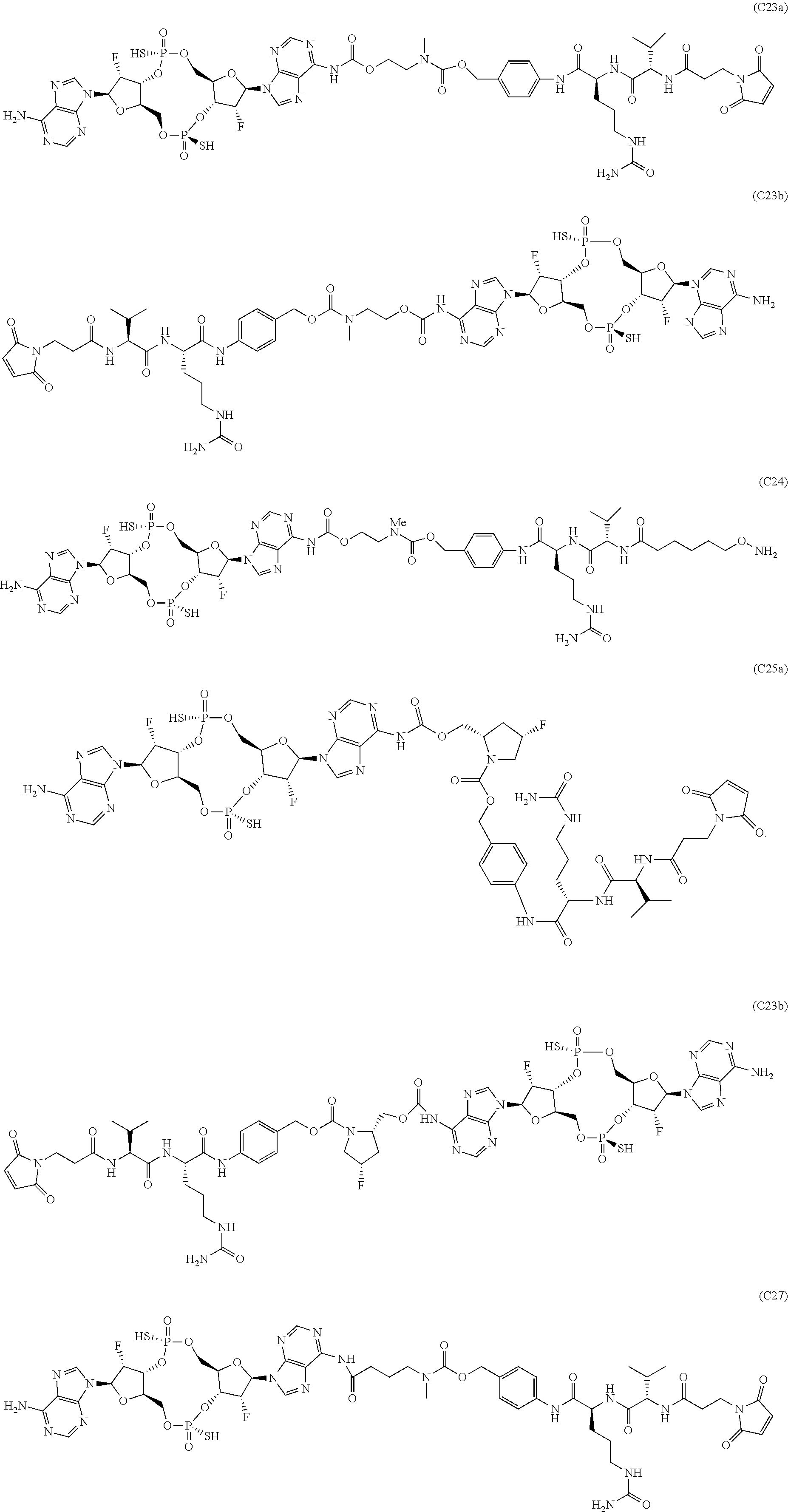

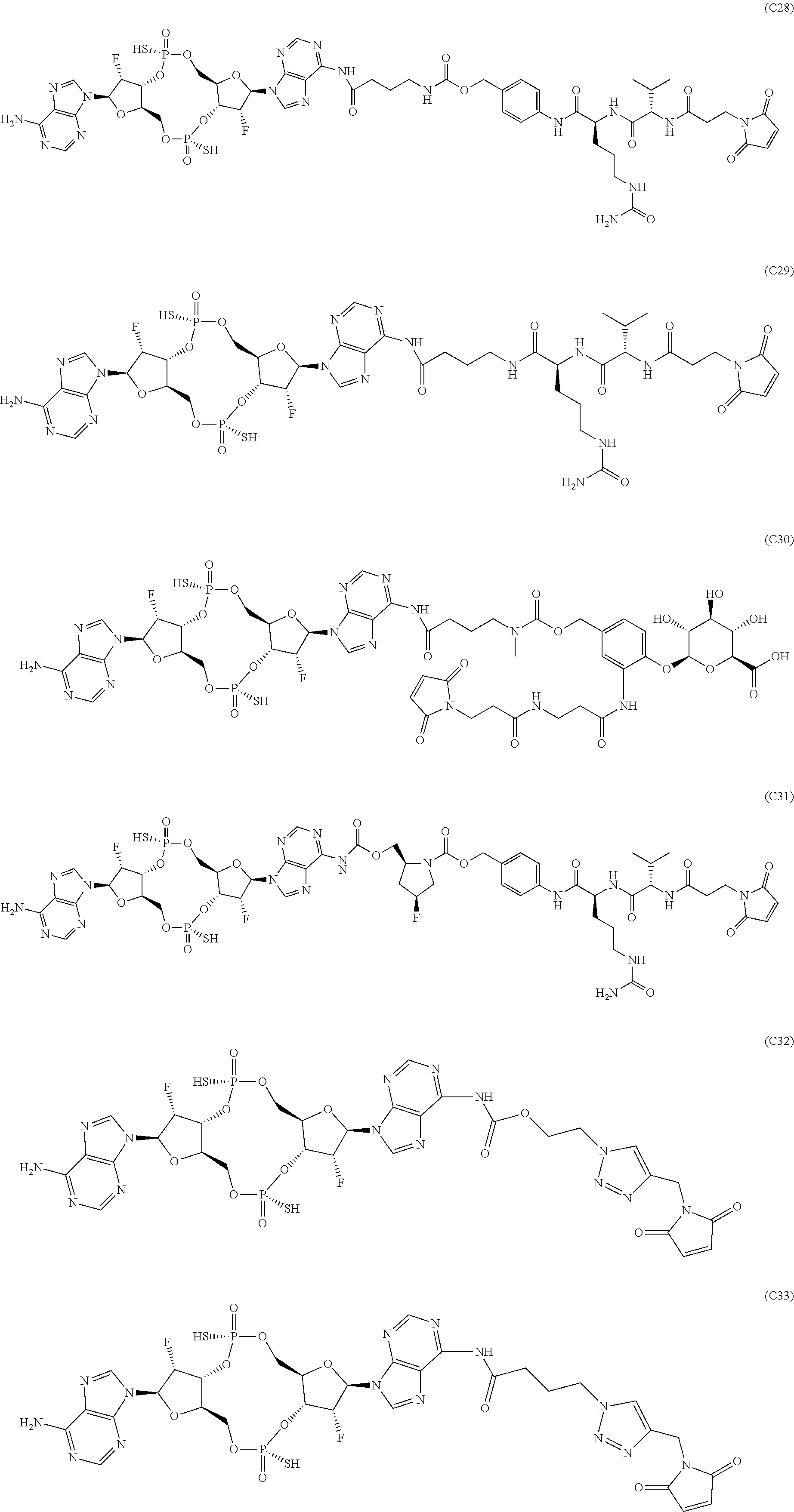

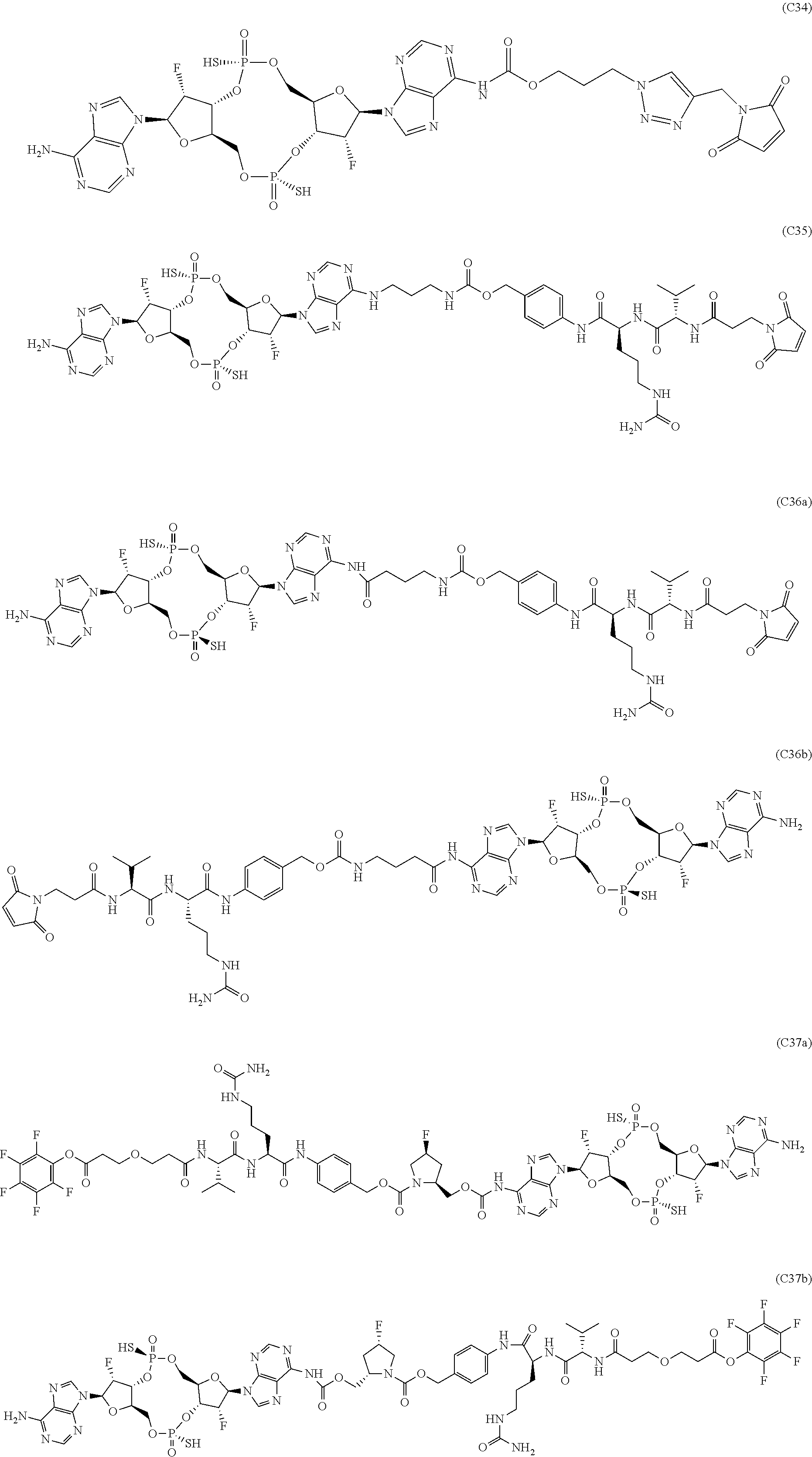

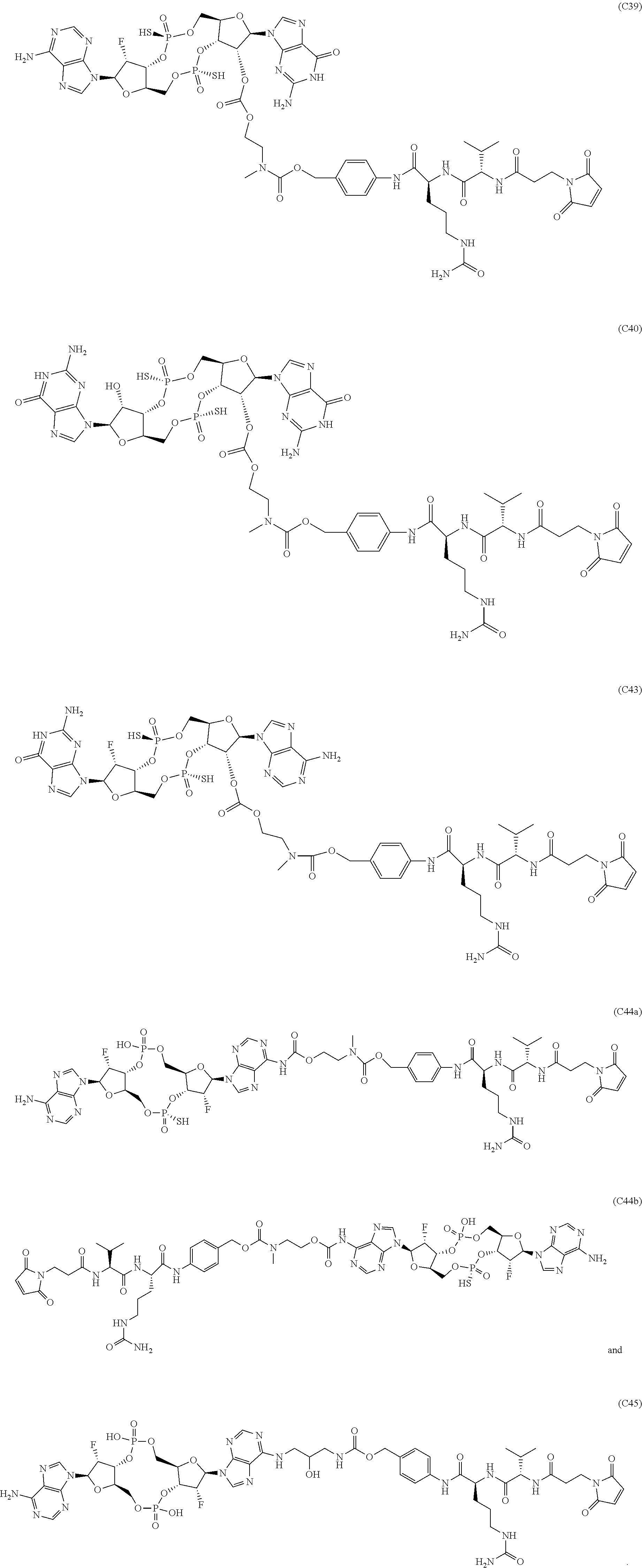

### Embodiment 187

A compound of Formula (B) selected from:

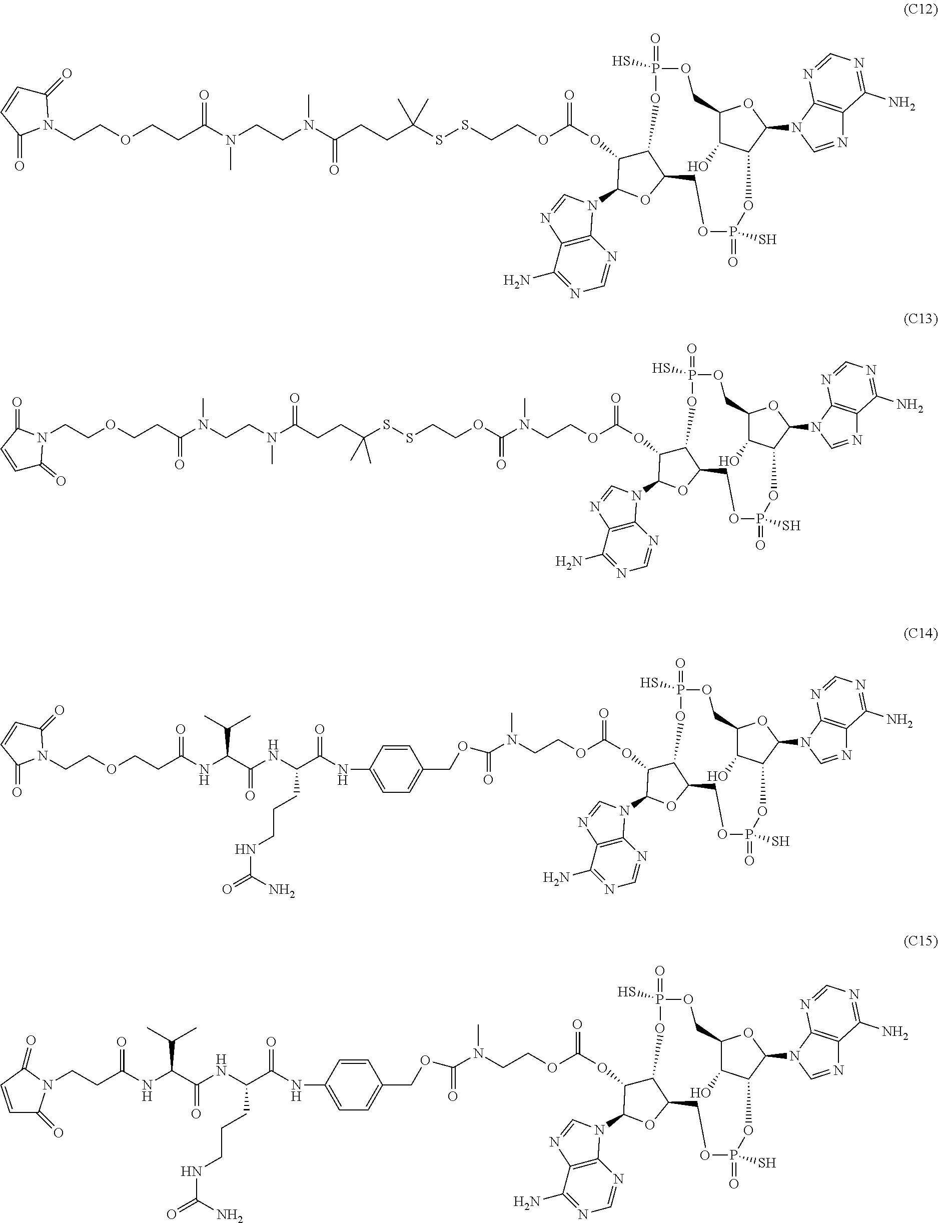

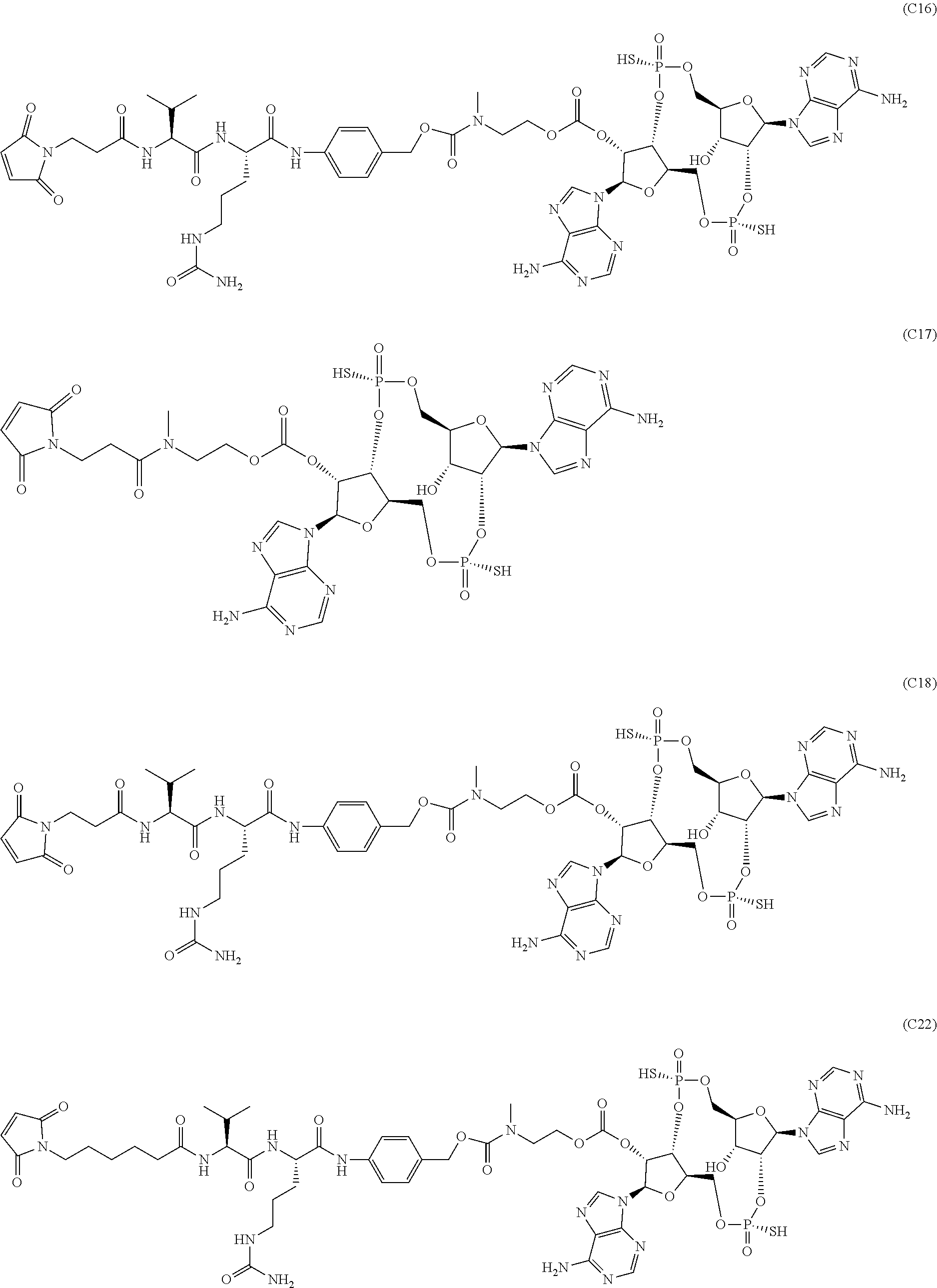

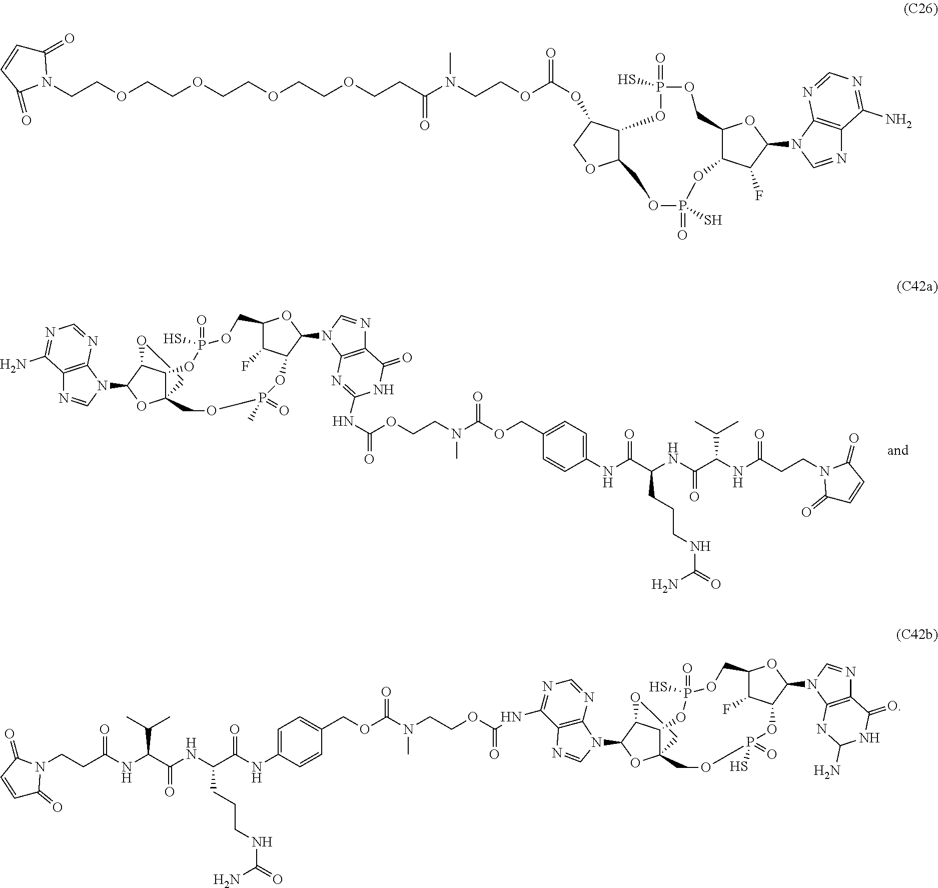

### Methods of Conjugation

The present invention provides various methods of conjugating Linker-Drug moieties to antibodies or antibody fragments to produce antibody drug conjugates, also referred to as immunconjugates.

A general reaction scheme for the formation of immunostimmulator antibody conjugates of Formula (I) is shown in Scheme 1 below:

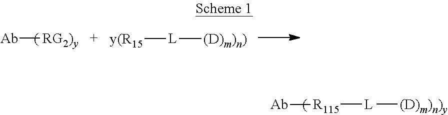

where: RG₂ is a reactive group which reacts with a compatible R¹⁵ group to form a corresponding R¹¹⁵ group (such groups are illustrated in Table 5). D, R¹⁵, L, Ab, y, m, n and R¹¹⁵ are as defined herein.

Scheme 2 further illustrates this general approach wherein the antibody comprises reactive groups (RG₂) which react with an R¹⁵ group (as defined herein) to covalently attach the Linker-Drug moiety to the antibody via an R¹¹⁵ group (as defined herein). For illustrative purposes only Scheme 2 shows the antibody having four RG₂ groups.

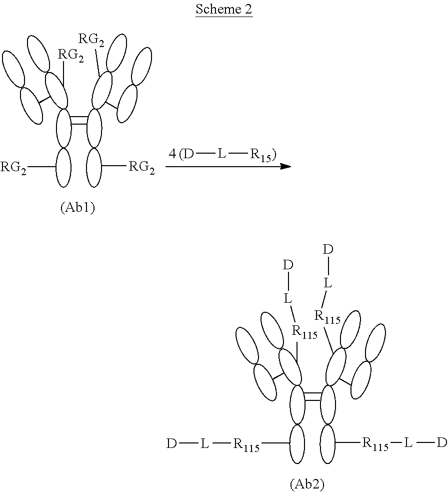

In one aspect, Linker-Drug moieties are conjugated to antibodies via modified cysteine residues in the antibodies (see for example WO2014/124316). Scheme 3 illustrates this approach wherein a free thiol group generated from the engineered cysteine residues in the antibody react with an R¹⁵ group (where R¹⁵ is a maleimide) to covalently attach the Linker-Drug moiety to the antibody via an R¹¹⁵ group (where R¹¹⁵ is a succinimide ring). For illustrative purposes only Scheme 3 shows the antibody chaving four free thiol groups.

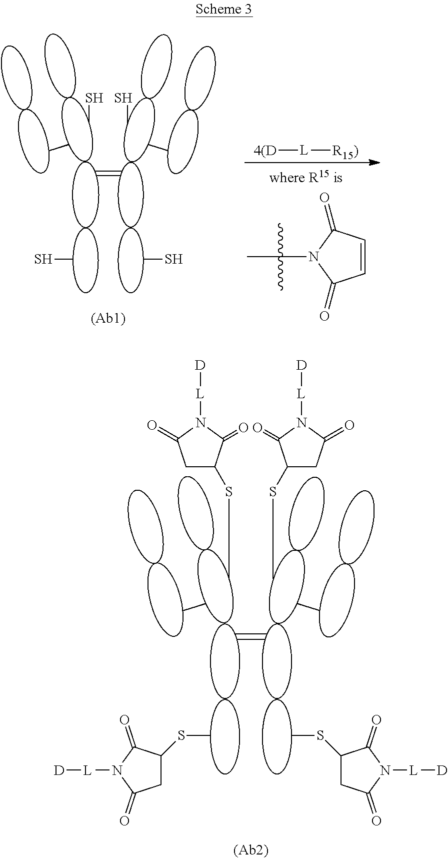

In another aspect, Linker-Drug moieties are conjugated to antibodies via lysine residues in the antibodies. Scheme 4 illustrates this approach wherein a free amine group from the lysine residues in the antibody react with an R¹⁵ group (where R¹⁵ is an NHS ester, a pentafluorophenyl or a tetrafluorophenyl) to covalently attach the Linker-Drug moiety to the antibody via an R¹¹⁵ group (where R¹¹⁵ is an amide). For illustrative purposes only Scheme 4 shows the antibody chaving four amine groups.

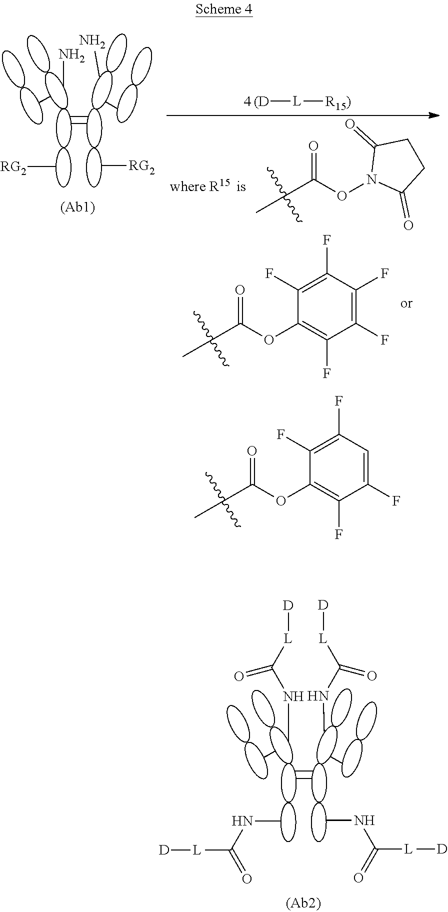

In another aspect, Linker-Drug moieties are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug moiety comprising a hydroxyl amine thereby form an oxime linkage (oxime bridge) which attaches the Linker-Drug moiety to the antibody (see for example WO2014/083505). Scheme 5 illustrates this approach.

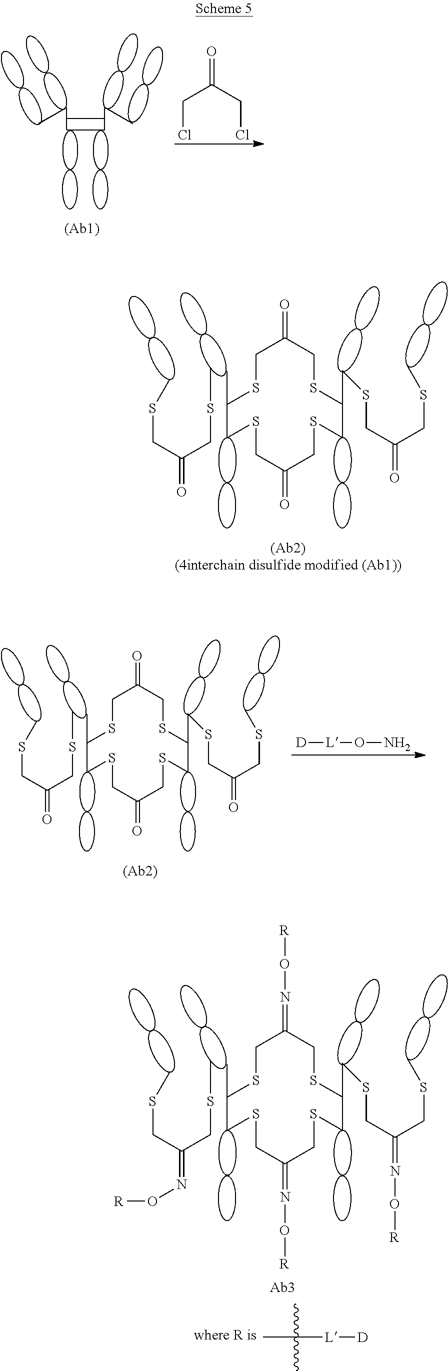

In yet another aspect, Linker-Drug moieties are conjugated to antibodies by inserting a peptide tag containing a serine residue, such as an S6, ybbR, or Al tag, into the sequence of an antibody as described in Bioconjugate Chemistry, 2015, 26, 2554-2562. These tags acts as a substrate for 4′-phosphopantetheinyl transferases (PPTase) enzymes wherein the PPTase posttranslationally modifies the serine residue to covalently attach a linker derived from coenzyme A (CoA) or from CoA analogues. The linker comprises a pendent ketone which is subsequently reacted with a Linker-Drug moiety comprising a hydroxyl amine thereby forming an oxime linkage which attaches the Linker-Drug moiety to the antibody. Scheme 6 illustrates this approach.

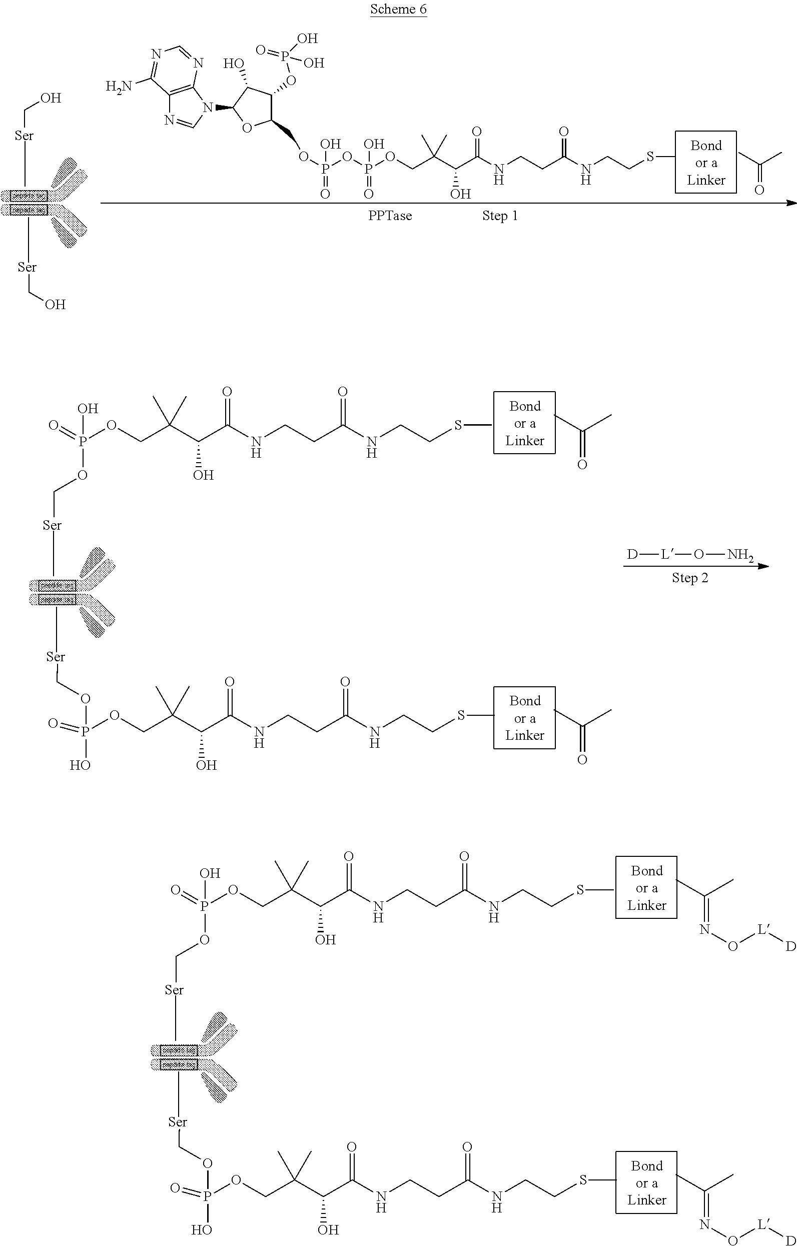

### DC-SIGN Immunoconjugates of the Invention

The present invention provides DC-SIGN immunoconjugates, also referred to as antibody drug conjugates, where an anti-DC-SIGN antibody, or a functional fragment thereof, is coupled to an agonist of STING via a linker. The DC-SIGN immunoconjugates of the invention can deliver an effective dose of a STING agonist to DC-SIGN+ cells, such as dendritic cells (DCs) and/or macrophages. In some embodiments, the DC-SIGN immunoconjugates of the invention can deliver an effective dose of a STING agonist to tumor residing antigen presenting cells, such as tumor residing DCs and/or macrophages, whereby stimulates activation of the DC-SIGN expressing cells and triggers an immune response including tumor specific T cell activation, in the tumor. The DC-SIGN immunoconjugates can also deliver an effective dose of a STING agonist to lymphoid tissue resident and peripheral tissue resident DC-SIGN expressing cells, including dendritic cells and macrophages. Delivery of the DC-SIGN immunoconjugates to DC-SIGN expressing cells not located in the tumor also stimulates activation of the DC-SIGN expressing cells and triggers an immune response.

In one aspect, the anti-DC-SIGN antibodies, antigen binding fragments or their functional equivalents of the invention are linked, via covalent attachment by a linker, to one or more compounds that are agonists of Stimulator of Interferon Genes (STING) receptor.

In one aspect, the anti-DC-SIGN antibodies, antigen binding fragments or their functional equivalents of the invention are linked, via covalent attachment by a linker, to one or more compounds that are cyclic dinucleotides which bind to Stimulator of Interferon Genes (STING) receptor.

In one aspect, the anti-DC-SIGN antibodies, antigen binding fragments or their functional equivalents of the invention are linked, via covalent attachment by a linker, to one or more compounds that are cyclic dinucleotides which are agonists of Stimulator of Interferon Genes (STING) receptor.

In one aspect, the anti-DC-SIGN immunoconjugates of the invention comprises one or more Drug moieties (D) as described herein.

In one aspect, the anti-DC-SIGN immunoconjugates of the invention comprises one or more Drug moieties (D), wherein the Drug moiety (D) is a compound which binds to Stimulator of Interferon Genes (STING) receptor and which comprises one or more reactive moieties capable of forming a covalent bond with one or more linker(s) (L).

In one aspect, the anti-DC-SIGN immunoconjugates of the invention comprises one or more Drug moieties (D), wherein the Drug moiety (D) is a compound which binds to Stimulator of Interferon Genes (STING) receptor and which a comprises one or more reactive moieties capable of forming a covalent bond with one or more linker(s) (L), wherein linker (L) is a cleavable linker.

In one aspect, the anti-DC-SIGN immunoconjugates of the invention comprise one or more Drug moieties (D), wherein the Drug moiety (D) is a dinucleotide which binds to Stimulator of Interferon Genes (STING) receptor and which comprises one or more reactive moieties capable of forming a covalent bond with one or more linker(s) (L).

In one aspect, the anti-DC-SIGN immunoconjugates of the invention, comprises one or more Drug moieties (D), wherein the Drug moiety (D) is a dinucleotide which binds to Stimulator of Interferon Genes (STING) receptor and which comprises one or more reactive moieties capable of forming a covalent bond with one or more linker(s) (L), wherein linker (L) is a cleavable linker.

In one aspect, the anti-DC-SIGN immunoconjugates of the invention comprise one or more Drug moieties (D), wherein the Drug moiety (D) is a cyclic dinucleotide which binds to Stimulator of Interferon Genes (STING) receptor and which comprises one or more reactive moieties capable of forming a covalent bond with one or more linker(s) (L).

In one aspect, the anti-DC-SIGN immunoconjugates of the invention, comprise one or more Drug moieties (D), wherein the Drug moiety (D) is a cyclic dinucleotide which binds to Stimulator of Interferon Genes (STING) receptor and which comprises one or more reactive moieties capable of forming a covalent bond with one or more linker(s) (L), wherein linker (L) is a cleavable linker.

In one aspect, the invention provides an immunoconjugate of Formula (I):

Ab-(L-(D)ₘ)ₙ (Formula(I))

wherein:
Ab is an anti-DC-SIGN antibody or fragment thereof;
L is a linker comprising one or more cleavage elements;
D is a compound which binds to Stimulator of Interferon Genes (STING) receptor;
m is an integer from 1 to 8; and
n is an integer from 1-20.

In another aspect, the invention provides an immunoconjugate of Formula (II):

Ab-(L-D)ₙ (Formula(II))

wherein:
Ab is an anti-DC-SIGN antibody or fragment thereof;
L is a linker comprising one or more cleavage elements;
D is a compound which binds to Stimulator of Interferon Genes (STING) receptor;
and
n is an integer from 1-20.

In another aspect, the invention provides an immunoconjugate of Formula (I):

Ab-(L-(D)ₘ)ₙ (Formula (I)

wherein:
Ab is an anti-DC-SIGN antibody or fragment thereof;
L is a linker comprising two or more cleavage elements;
D is a compound which binds to Stimulator of Interferon Genes (STING) receptor;
m is an integer from 1 to 8; and
n is an integer from 1-20.

In an embodiment of Formula (I) or Formula (II), D is an agonist of Stimulator of Interferon Genes (STING) receptor.

In an embodiment of Formula (I) or Formula (II), D is a cyclic dinucleotides which bind to Stimulator of Interferon Genes (STING) receptor.

In an embodiment of Formula (I) or Formula (II), D is a cyclic dinucleotide which is an agonist of Stimulator of Interferon Genes (STING) receptor.

In one aspect, the DC-SIGN immunoconjugates of the invention comprise one or more Drug moieties (D) as described herein.

In one aspect, the DC-SIGN immunoconjugates of the invention comprises one or more Drug moieties (D), wherein the Drug moiety (D) is a compound which binds to Stimulator of Interferon Genes (STING) receptor and which comprises one or more reactive moieties capable of forming a covalent bond with a linker.

In one aspect, the DC-SIGN immunoconjugates of the invention comprises one or more Drug moieties (D), wherein the Drug moiety (D) is a compound which binds to Stimulator of Interferon Genes (STING) receptor and which a comprises one or more reactive moieties capable of forming a covalent bond with a linker, wherein linker (L) is a cleavable linker.

In one aspect, the DC-SIGN immunoconjugates of the invention comprises one or more Drug moieties (D), wherein the Drug moiety (D) is a dinucleotide which binds to Stimulator of Interferon Genes (STING) receptor and which comprises one or more reactive moieties capable of forming a covalent bond with a linker.

In one aspect, the DC-SIGN immunoconjugates of the invention, comprises one or more Drug moieties (D), wherein the Drug moiety (D) is a dinucleotide which binds to Stimulator of Interferon Genes (STING) receptor and which comprises one or more reactive moieties capable of forming a covalent bond with a linker, wherein linker (L) is a cleavable linker.

In one aspect, the DC-SIGN immunoconjugates of the invention comprise one or more Drug moieties (D), wherein the Drug moiety (D) is a cyclic dinucleotide which binds to Stimulator of Interferon Genes (STING) receptor and which comprises one or more reactive moieties capable of forming a covalent bond with a linker.

In one aspect, the DC-SIGN immunoconjugates of the invention, comprise one or more Drug moieties (D), wherein the Drug moiety (D) is a cyclic dinucleotide which binds to Stimulator of Interferon Genes (STING) receptor and which comprises one or more reactive moieties capable of forming a covalent bond with a linker, wherein linker (L) is a cleavable linker.

The term “cleavage product”, as used herein, refers to a drug moiety (D) linked to a fragment of the linker wherein the fragment comprises one or more linker components (Lc). The cleavage product is formed upon cleavage of Linker (L) from Ab-(L-(D)ₘ)ₙ, wherein a fragment of the Linker (L) remains attached to the drug moiety (D).

In one embodiment, the DC-SIGN immunoconjugates of the invention comprise Formula (I):

Ab-(L-(D)ₘ)ₙ (Formula(I))

wherein:
Ab is an anti DC-SIGN antibody or a functional fragment thereof;
L is a linker comprising one or more cleavage elements;
D is a drug moiety] that has agonist activity against STING receptor;
m is an integer from 1 to 8; and
n is an integer from 1 to 20.

In one embodiment, the DC-SIGN immunoconjugates of the invention comprise Formula (I):

Ab-(L-(D)ₘ)ₙ (Formula(I))

wherein:
Ab is an anti DC-SIGN antibody or a functional fragment thereof;
L is a linker;
D is a drug moiety that binds to STING receptor;
m is an integer from 1 to 8; and
n is an integer from 1 to 20;
   and wherein D, or a cleavage product thereof, that is released from the DC-SIGN immunoconjugate has STING agonist activity.

In one embodiment, the DC-SIGN immunoconjugates of the invention comprise Formula (I):

Ab-(L-(D)ₘ)ₙ (Formula(I))

wherein:
Ab is an anti DC-SIGN antibody or a functional fragment thereof;
L is a linker;
D is a drug moiety that binds to STING receptor;
m is an integer from 1 to 8; and
n is an integer from 1 to 20;
   wherein the DC-SIGN immunoconjugate delivers D, or a cleavage product thereof, to a cell targeted by the Ab, and wherein D, or the cleavage product thereof, has STING agonist activity.

In one embodiment, the DC-SIGN immunoconjugates of the invention comprise Formula (I):

Ab-(L-(D)ₘ)ₙ (Formula (I))

wherein:
Ab is an anti DC-SIGN antibody or a functional fragment thereof;
L is a linker comprising one or more cleavage elements;
D is a drug moiety that binds to STING receptor;
m is an integer from 1 to 8; and
n is an integer from 1 to 20;
   and wherein the DC-SIGN immunoconjugate releases D, or a cleavage product thereof, in a cell targeted by the Ab, and wherein D, or the cleavage product thereof, has STING agonist activity.

In one embodiment, the DC-SIGN immunoconjugates of the invention comprise Formula (I):

Ab-(L-(D)ₘ)ₙ (Formula (I))

wherein:
Ab is an anti DC-SIGN antibody or a functional fragment thereof;
L is a linker comprising one or more cleavage elements;
D is a drug moiety that has agonist activity against STING receptor;
m is an integer from 1 to 8; and
n is an integer from 1 to 20;
   wherein the DC-SIGN immunoconjugate releases D, or a cleavage product thereof, in a cell targeted by the Ab, and wherein D, or the cleavage product thereof, has STING agonist activity in the cell.

In one embodiment, the DC-SIGN immunconjugates of the invention comprise Formula (I):

Ab-(L-(D)ₘ)ₙ (Formula (I))

wherein:
Ab is an anti-DC-SIGN antibody or a functional fragment thereof;
L is a linker comprising one or more cleavage elements;
D is a drug moiety that binds to STING receptor;
m is an integer from 1 to 8; and
n is an integer from 1 to 20;
   wherein the DC-SIGN immunoconjugate specifically binds to DC-SIGN expressed on the cell surface and is internalized into the cell, and wherein D, or a cleavage product thereof, is cleaved from L and has STING agonist activity as determined by one or more STING agonist assays selected from: an interferon stimulation assay, a hSTING wt assay, a THP1-Dual assay, a TANK binding kinase 1 (TBK1) assay, or an interferon-γ-inducible protein (IP-10) secretion assay.

In one aspect the DC-SIGN immunoconjugate of the invention, the DC-SIGN immunoconjugate is selected from the following;

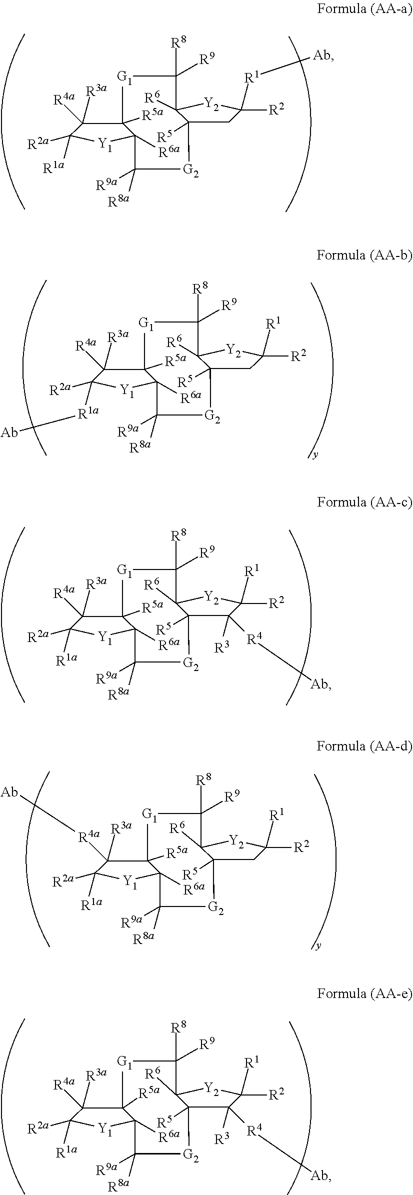

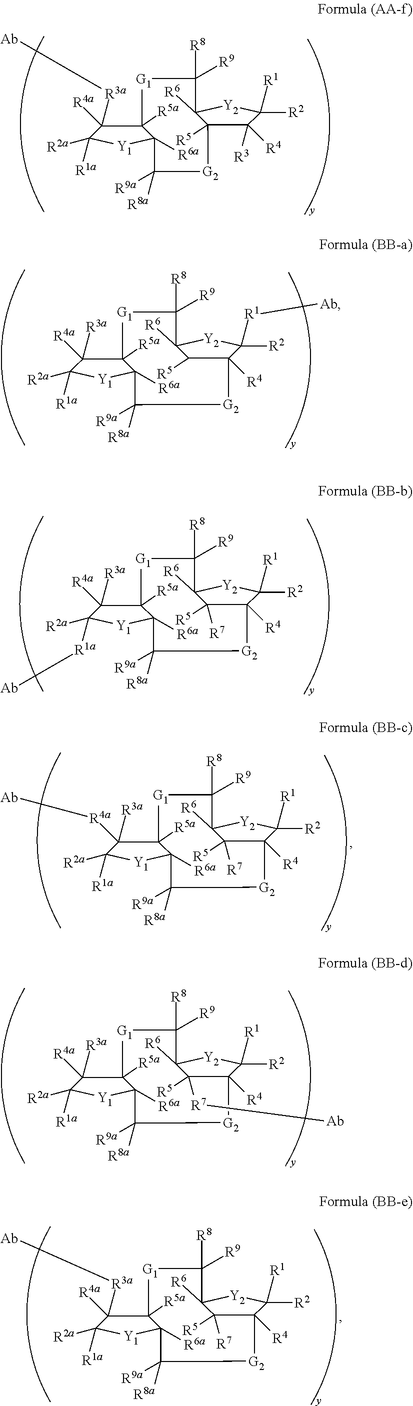

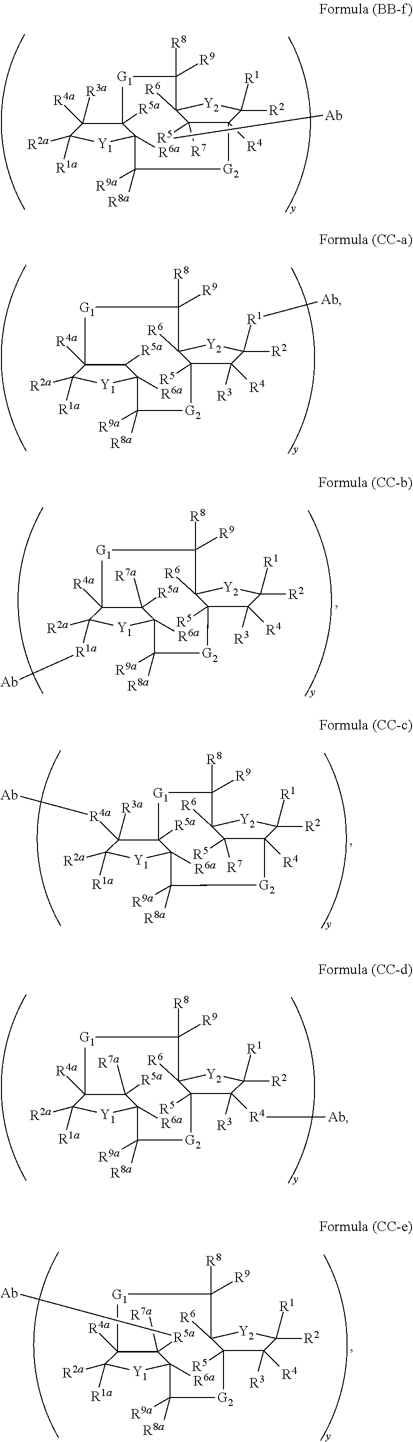

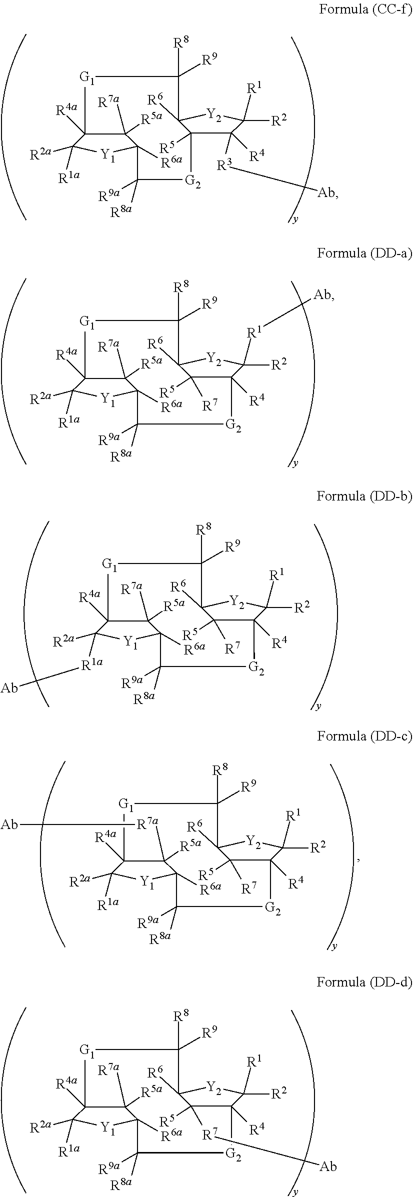

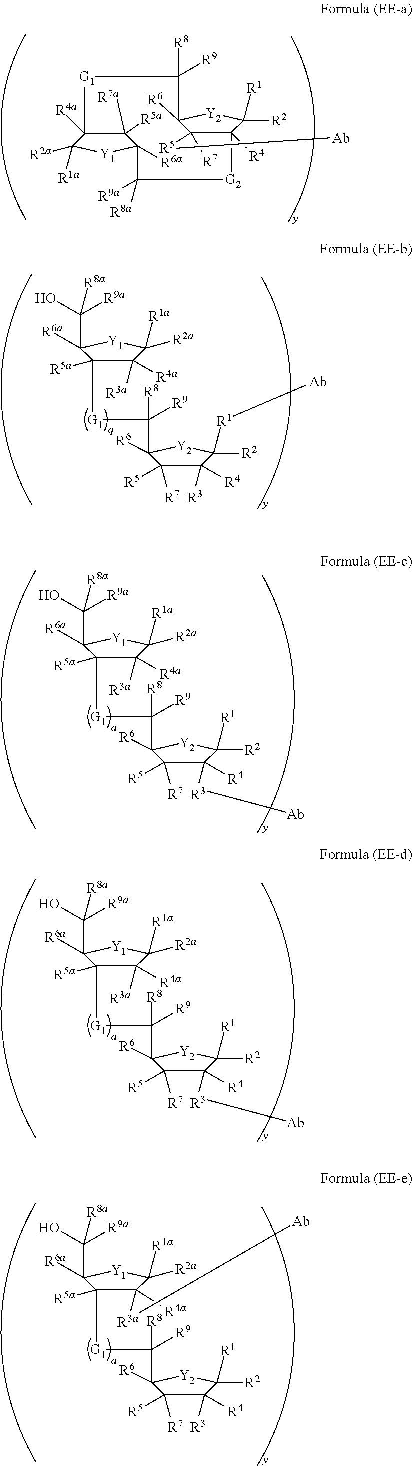

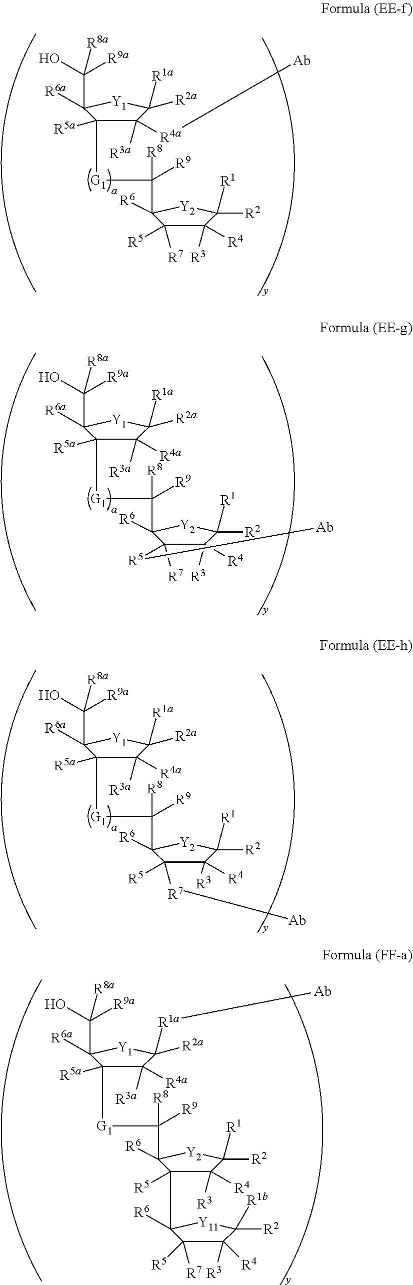

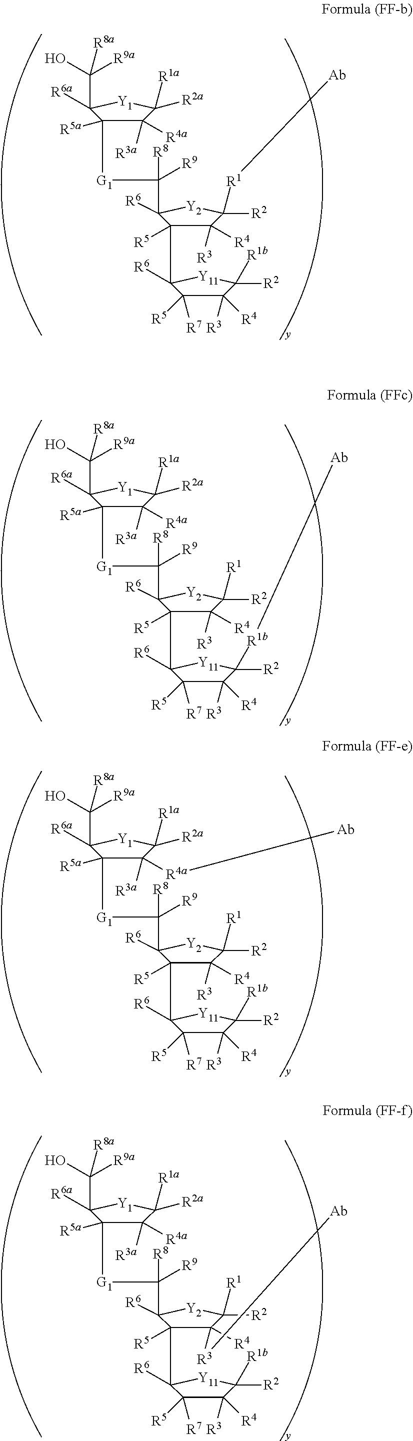

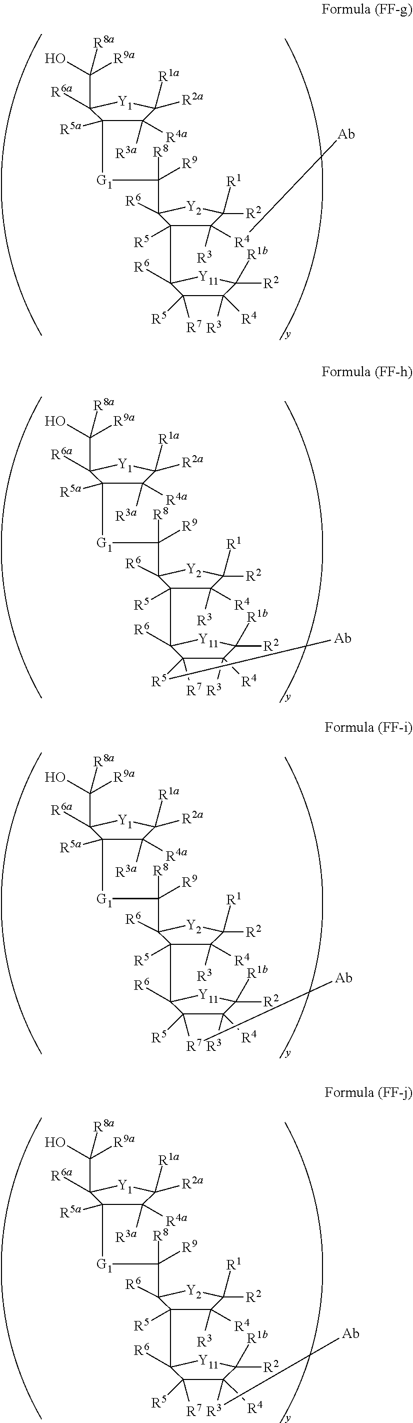

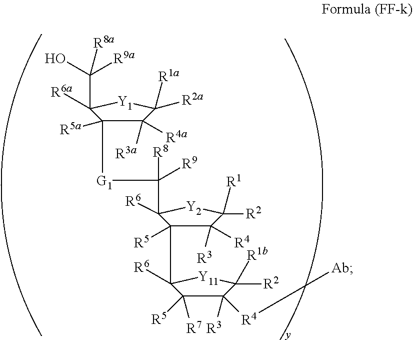

wherein:
each G₁ is independently selected from

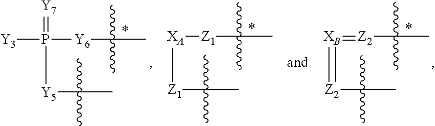

where the * of G₁ indicates the point of attachment to —CR⁸R⁹—;
X_{A} is C(═O)—, —C(═S)— or —C(═NR¹)— and each Z₁ is NR¹²;
X_{B} is C, and each Z₂ is N;
G₂ is

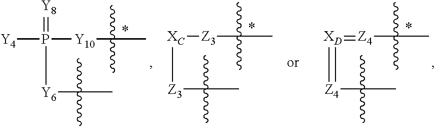

where the * of G₂ indicates the point of attachment to —CR^{8a}R^{9a}—;
X_{C} is C(═O)—, —C(═S)— or —C(═NR¹¹)— and each Z₃ is NR¹²;
X_{D} is C, and each Z₄ is N;
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₃ is OH, O⁻, OR¹⁰, N(R¹⁰)₂, SR¹⁰, SeH, Se⁻, BH₃, SH or S⁻;
Y₄ is OH, O⁻, OR¹⁰, N(R¹⁰)₂, SR¹⁰, SeH, Se⁻, BH₃, SH or S⁻;
Y₅ is —CH₂—, —NH—, —O— or —S;
Y₆ is —CH₂—, —NH—, —O— or —S;
Y₇ is O or S;
Y₈ is O or S;
Y₉ is —CH₂—, —NH—, —O— or —S;
Y₁₀ is —CH₂—, —NH—, —O— or —S;
Y₁₁ is —O—, —S—, —S(═O)—, —SO₂—, —CH₂—, or —CF₂—;
q is 1, 2 or 3;
each R¹ is independently a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R¹ is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R^{1a} is independently a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1a} is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R^{1b} is independently a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1b} is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
each R⁸ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁸ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R⁹ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁹ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁹ are substituted by 0, 1, 2 or 3 substituents independently selected from F, Cl, Br, I, OH, CN, and N₃;
each R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
each R^{3a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
each R^{4a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
each R^{5a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
each R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
each R^{7a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
each R^{8a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{8a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R^{9a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{9a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{9a} are substituted by 0, 1, 2 or 3 substituents independently selected from F, Cl, Br, I, OH, CN, and N₃;
each R¹⁰ is independently selected from the group consisting of H, C₁-C₁₂alkyl, C₁-C₆heteroalkyl, —(CH₂CH₂O)ₙCH₂CH₂C(═O)OC₁-C₆alkyl, and

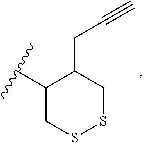 [SMILES: *C1CSSCC1CC#C]

wherein the C₁-C₁₂alkyl and C₁-C₆heteroalkyl of R¹⁰ is substituted by 0, 1, 2 or 3 substituents independently selected from —OH, C₁-C₁₂alkoxy, —S—C(═O)C₁-C₆alkyl, halo, —CN, C₁-C₁₂alkyl, —O-aryl, _O-heteroaryl, —O-cycloalkyl, oxo, cycloalkyl, heterocyclyl, aryl, or heteroaryl, —OC(O)OC₁-C₆alkyland C(O)OC₁-C₆alkyl, wherein each alkyl, cycloalkyl, heterocyclyl, aryl, and heteroaryl is substituted by 0, 1, 2 or 3 substituents independently selected from C₁-C₁₂ alkyl, O—C₁-C₁₂alkyl, C₁-C₁₂heteroalkyl, halo, CN, OH, oxo, aryl, heteroaryl, O-aryl, O-heteroaryl, —C(═O)C₁-C₁₂alkyl, —OC(═O)C₁-C₁₂alkyl, —C(═O)OC₁-C₁₂alkyl, —OC(═O)OC₁-C₁₂alkyl, —C(═O)N(R¹¹)—C₁-C₁₂alkyl, —N(R¹¹)C(═O)—C₁-C₁₂alkyl; —OC(═O)N(R¹¹)—C₁-C₁₂alkyl, —C(═O)-aryl, —C(═O)-heteroaryl, —OC(═O)-aryl, —C(═O)O-aryl, —OC(═O)-heteroaryl, —C(═O)O-heteroaryl, —C(═O)O-aryl, —C(═O)O-heteroaryl, —C(═O)N(R¹¹)-aryl, —C(═O)N(R¹¹)-heteroaryl, —N(R¹¹)C(O)-aryl, —N(R¹¹)₂C(O)-aryl, —N(R¹¹)C(O)-heteroaryl, and S(O)₂N(R¹¹)-aryl;
each R¹¹ is independently selected from H and C₁-C₆alkyl;
each R¹² is independently selected from H and C₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position;
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position;
optionally R⁸ and R⁹ are connected to form a C₁-C₆alkylene, C₂-C₆alkenylene, C₂-C₆alkynylene, and
optionally R^{8a} and R^{9a} are connected to form a C₁-C₆alkylene, C₂-C₆alkenylene, C₂-C₆alkynylene,
L₁ is a linker;
each R¹¹⁵ is independently

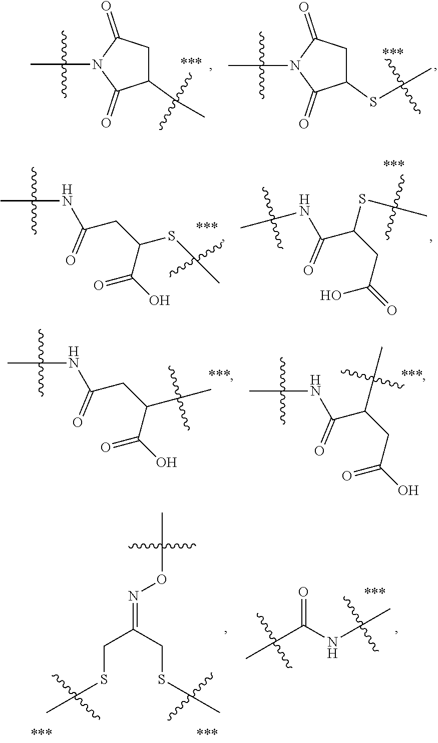

—C(═O)—, —ON═***, —S—, —NHC(═O)CH₂—***, —S(═O)₂CH₂CH₂—***, —(CH₂)₂S(═O)₂CH₂CH₂—***, —NHS(═O)₂CH₂CH_{2-**}, —NHC(═O)CH₂CH₂—***, —CH₂NHCH₂CH₂—***, —NHCH₂CH₂—***,

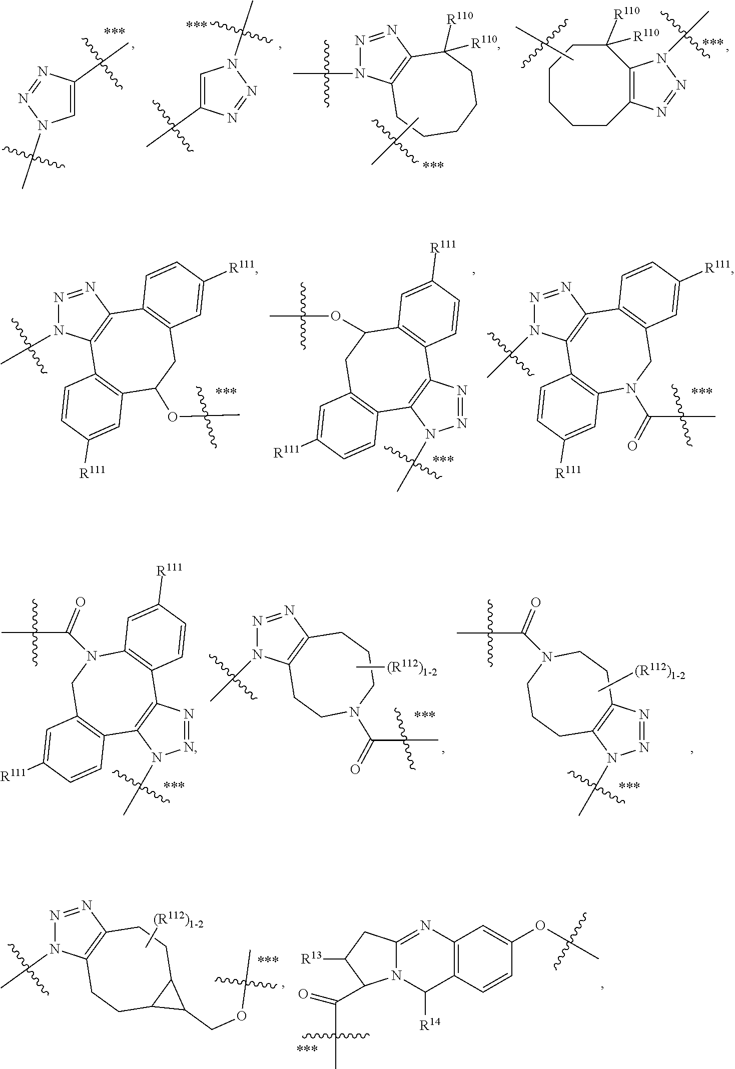

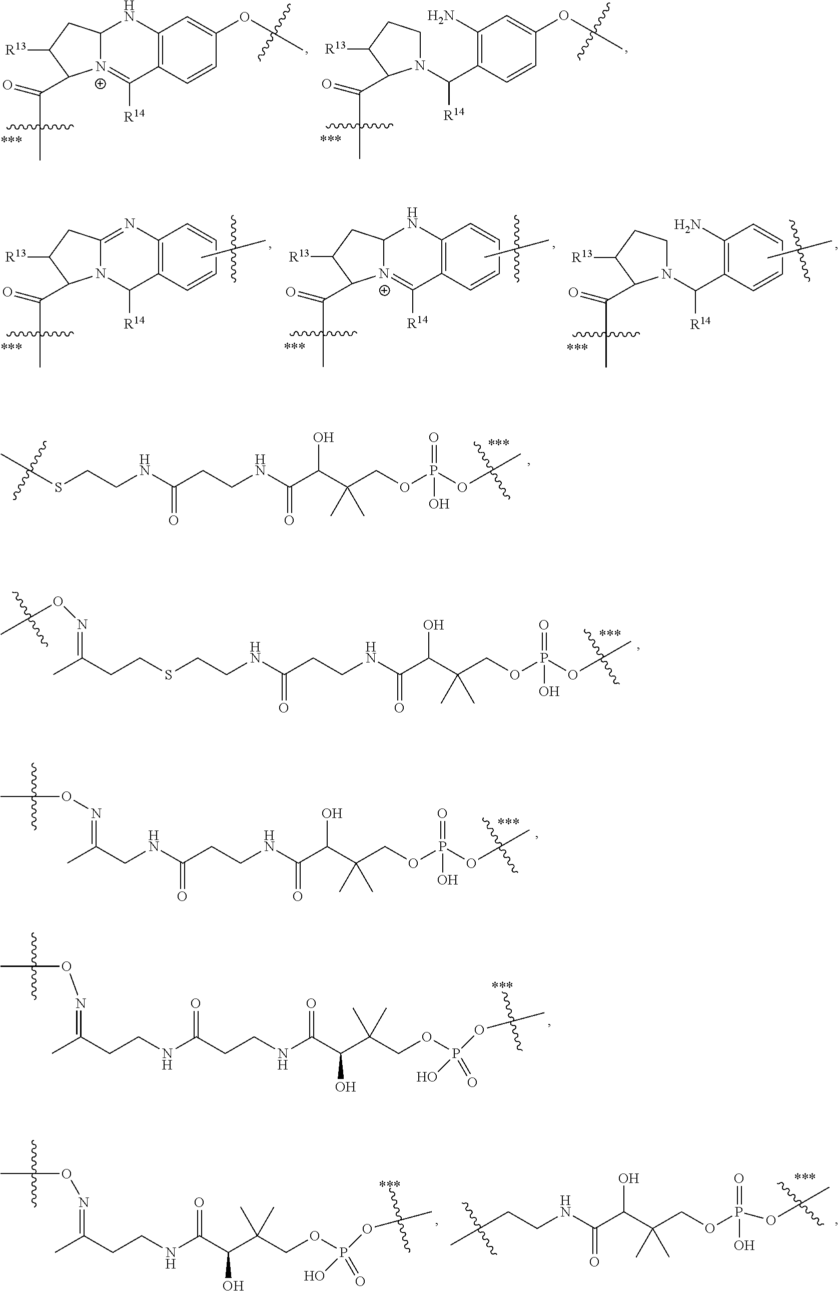

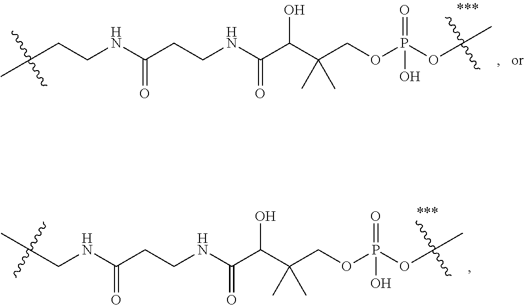 [SMILES: *CCNC(=O)CCNC(=O)C(O)C(C)(C)COP(=O)(O)O* ; *CNC(=O)CCNC(=O)C(O)C(C)(C)COP(=O)(O)O*]

where the *** of R¹¹⁵ indicates the point of attachment to Ab;
R¹³ is H or methyl;
R¹⁴ is H, —CH₃ or phenyl;
each R¹¹⁰ is independently selected from H, C₁-C₆alkyl, F, Cl, and —OH;
each R¹¹¹ is independently selected from H, C₁-C₆alkyl, F, Cl, —NH₂, —OCH₃, —OCH₂CH₃, —N(CH₃)₂, —CN, —NO₂ and —OH;
each R¹¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
Ab is an anti-DC-SIGN antibody or fragment thereof; and
y is 1, 2, 3, 4, 5, 6, 7, 8, 9 or 10,
and provided at least one of R¹, R^{1a} or R^{1b} is substituted with —NHL₁R¹¹⁵, or at least one of R³, R⁴, R⁵, R⁷, R^{3a}, R^{4a}, R^{5a} or R^{7a} is —OL₁R¹¹⁵.

Certain aspects and examples of the DC-SIGN Immunoconjugates of the invention are provided in the following listing of additional, enumerated embodiments. It will be recognized that features specified in each embodiment may be combined with other specified features to provide further embodiments of the present invention.

### Embodiment 188

The DC-SIGN immunoconjugate of Formulas (AA-a to AA-f), Formulas (BB-a to BB-f), Formulas (CC-a to CC-f), Formulas (DD-a to DD-f), Formulas (EE-a to EE-h) or Formulas (FF-a to FF-k), or stereoisomers or pharmaceutically acceptable salts thereof, wherein L₁ is a linker comprising one or more cleavage elements;

### Embodiment 189

A DC-SIGN immunoconjugate of Formulas (AA-a to AA-f), Formulas (BB-a to BB-f), Formulas (CC-a to CC-f), Formulas (DD-a to DD-f), Formulas (EE-a to EE-h) or Formulas (FF-a to FF-k), or stereoisomers or pharmaceutically acceptable salts thereof selected from:

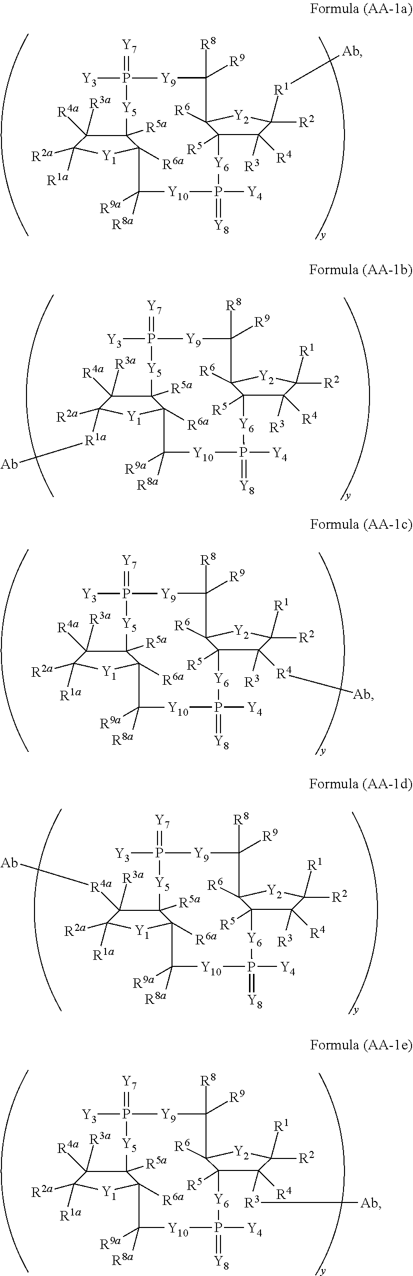

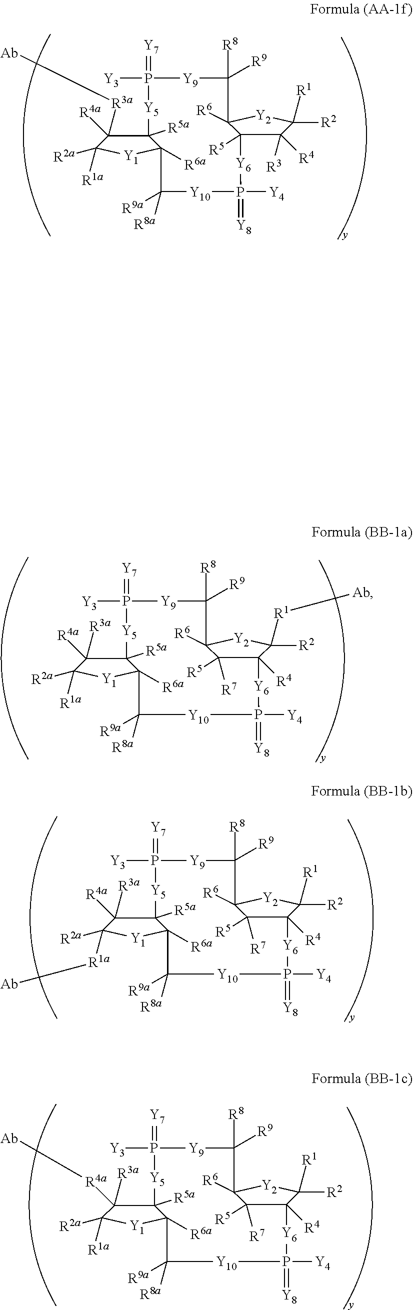

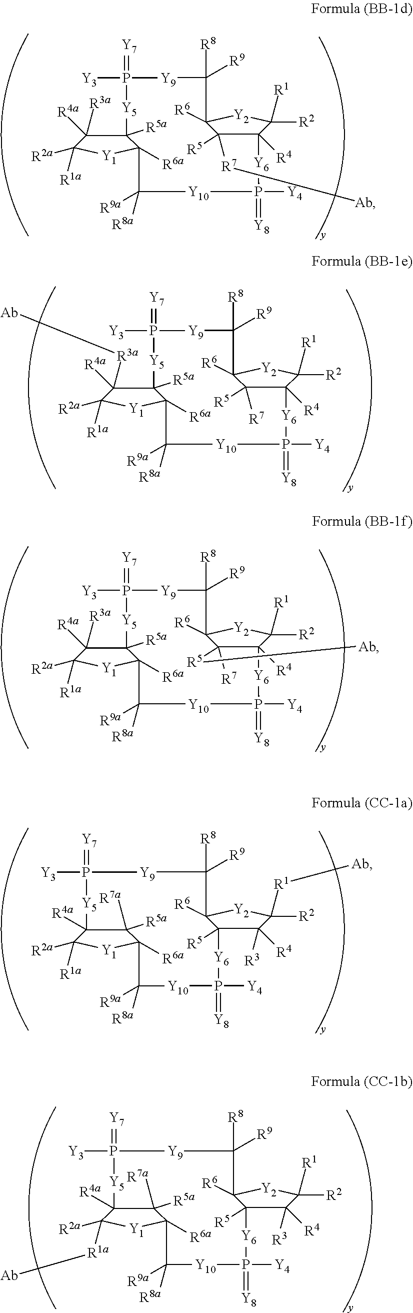

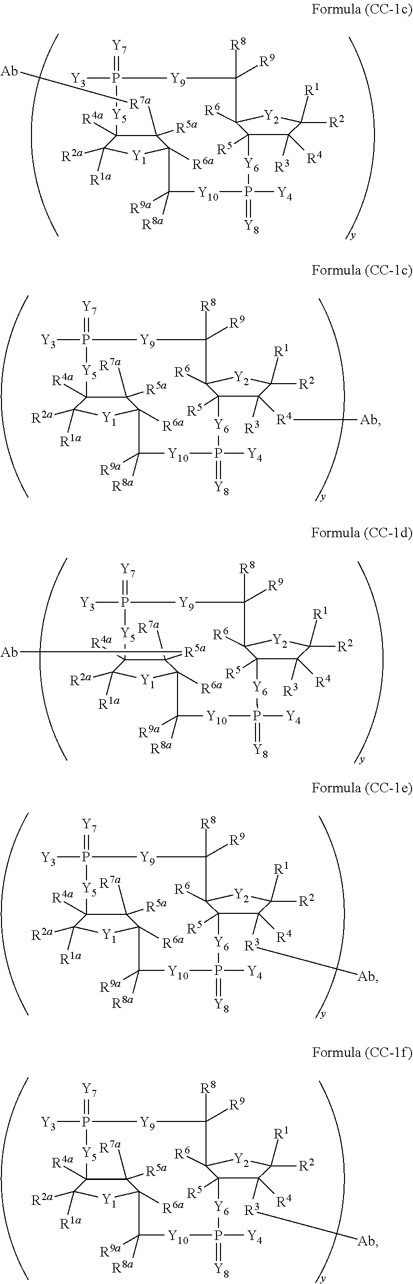

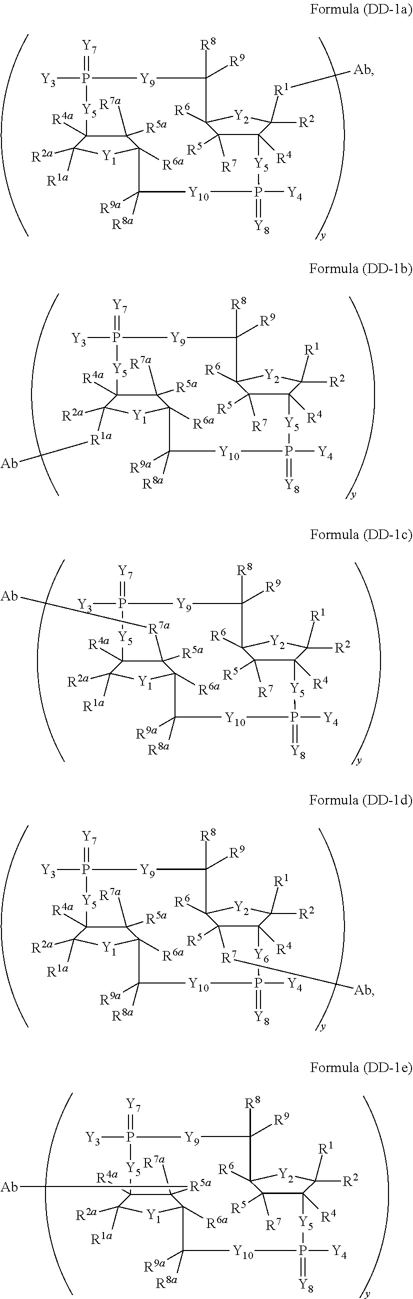

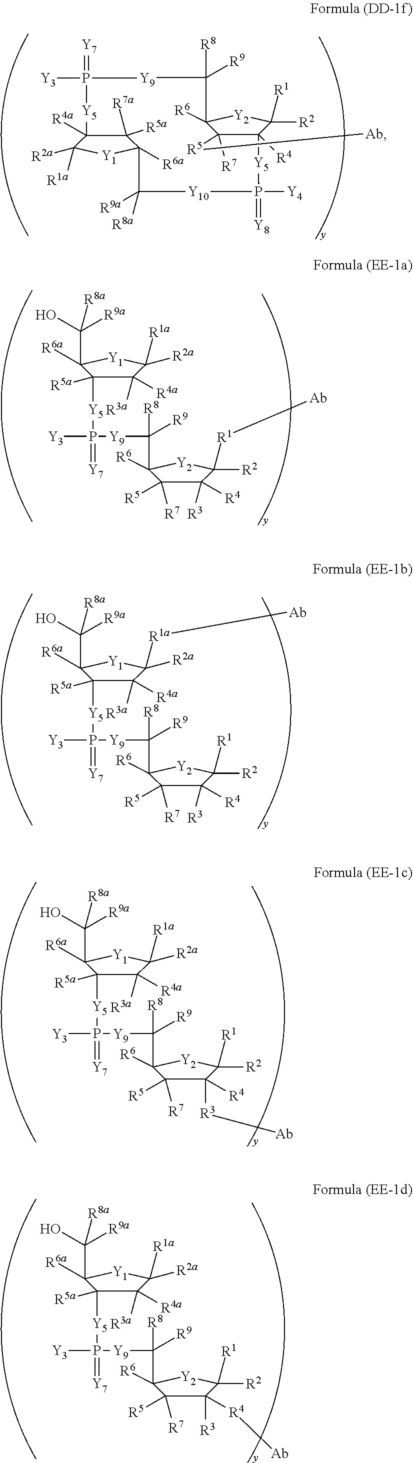

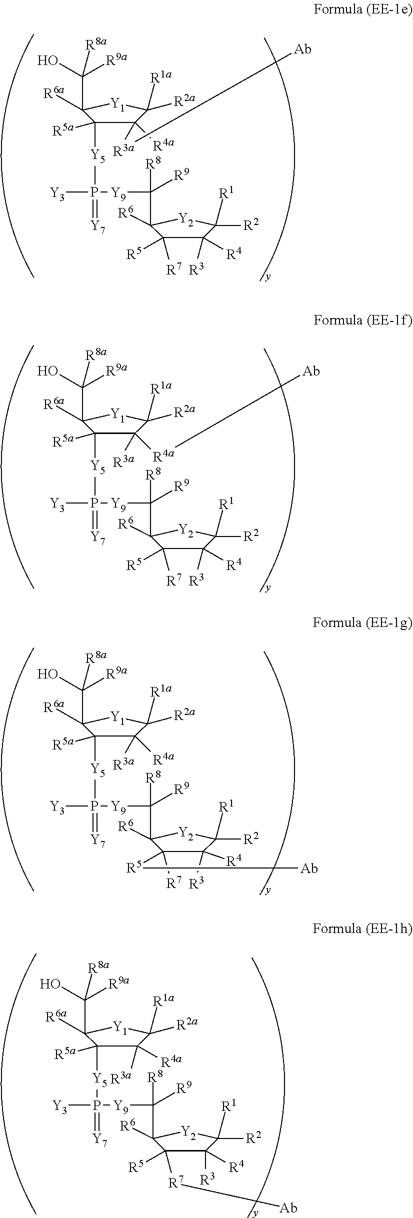

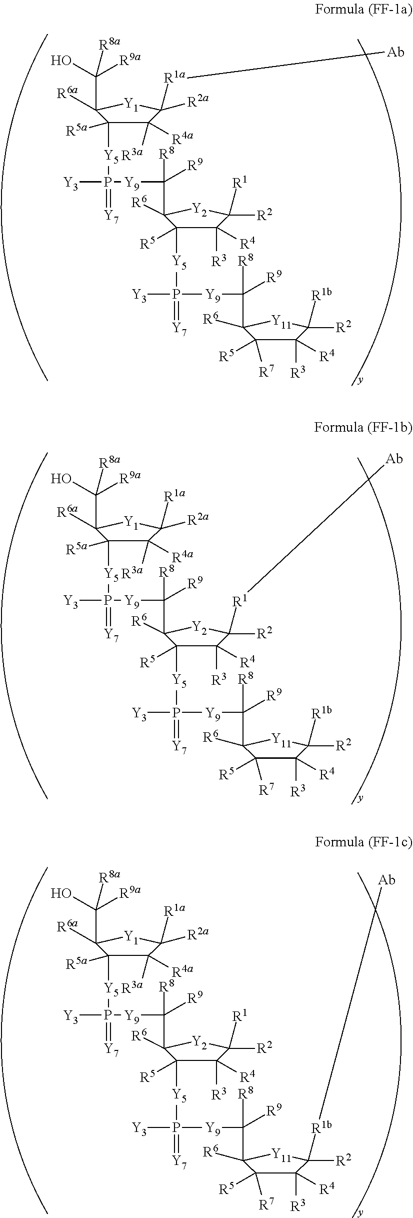

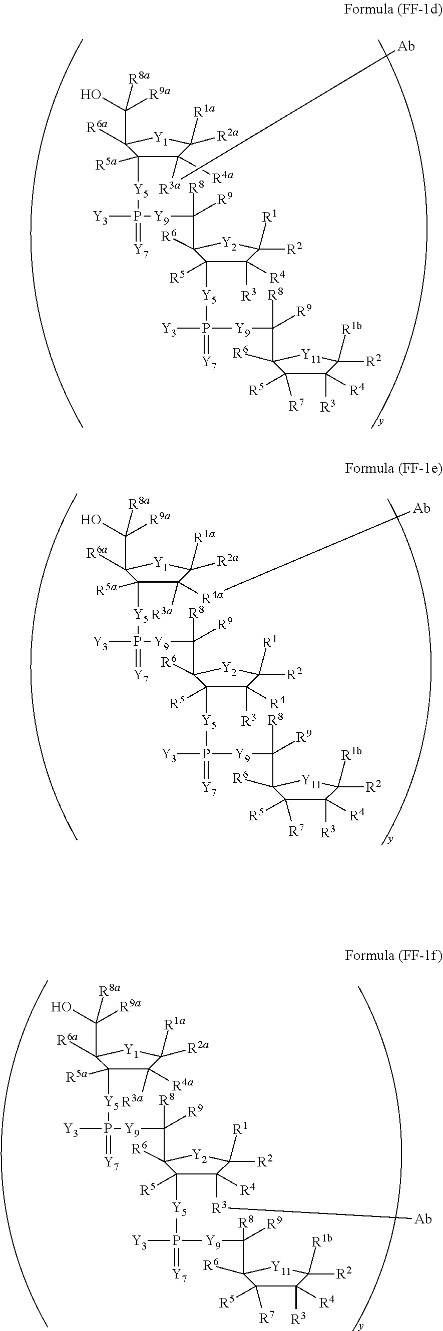

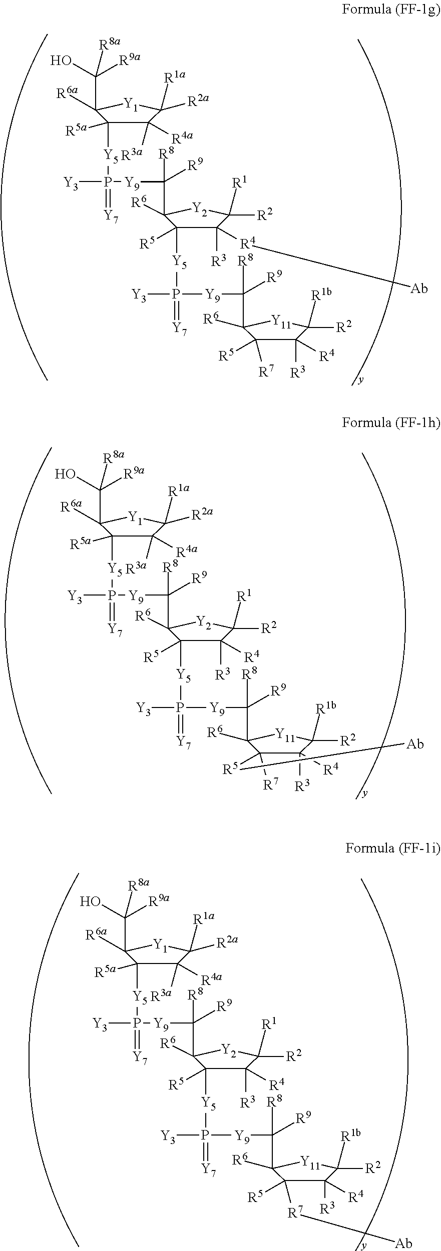

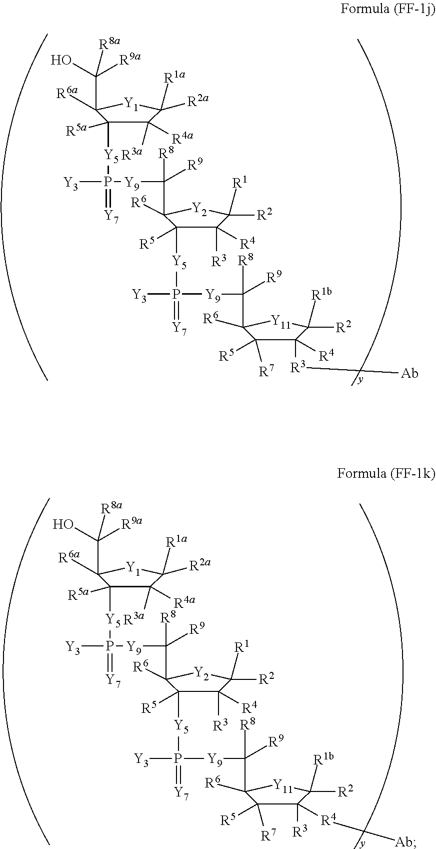

wherein y, Ab, R¹, R^{1a}, R^{1b}, R², R^{2a}, R³, R^{3a}, R⁴, R^{4a}, R⁵, R^{5a}, R⁶, R^{6a}, R⁷, R^{7a}, R⁸, R^{8a}, R⁹, R^{9a}, Y₁, Y₂, Y₃, Y₄, Y₅, Y₆, Y₇, Y₈, Y₉, Y₁₀ and Y₁₁ are as defined above for immunoconjugates of Formulas (AA-a to AA-f), Formulas (BB-a to BB-f), Formulas (CC-a to CC-f), Formulas (DD-a to DD-f), Formulas (EE-a to EE-h) and Formulas (FF-a to FF-k), and provided at least one of R¹, R^{1a} or Rib is substituted with —NHL₁R¹¹⁵, or at least one of R³, R⁴, R⁵, R⁷, R^{3a}, R^{4a}, R^{5a} or R^{7a} is —OL₁R¹¹⁵

### Embodiment 190

The DC-SIGN immunoconjugate of Embodiment 146, wherein R¹ is pyrimidine or purine nucleic acid base or analogue thereof, R^{1a} is pyrimidine or purine nucleic acid base or analogue thereof, and Rib is a pyrimidine or purine nucleic acid base or analogue thereof, each of which is substituted as described in R¹, R^{1a} or R^{1b} for immunoconjugates of Formulas (AA-a to AA-f), Formulas (BB-a to BB-f), Formulas (CC-a to CC-f), Formulas (DD-a to DD-f), Formulas (EE-a to EE-h) and Formulas (FF-a to FF-k).

### Embodiment 191

A DC-SIGN immunoconjugate of Embodiment 148 selected from:

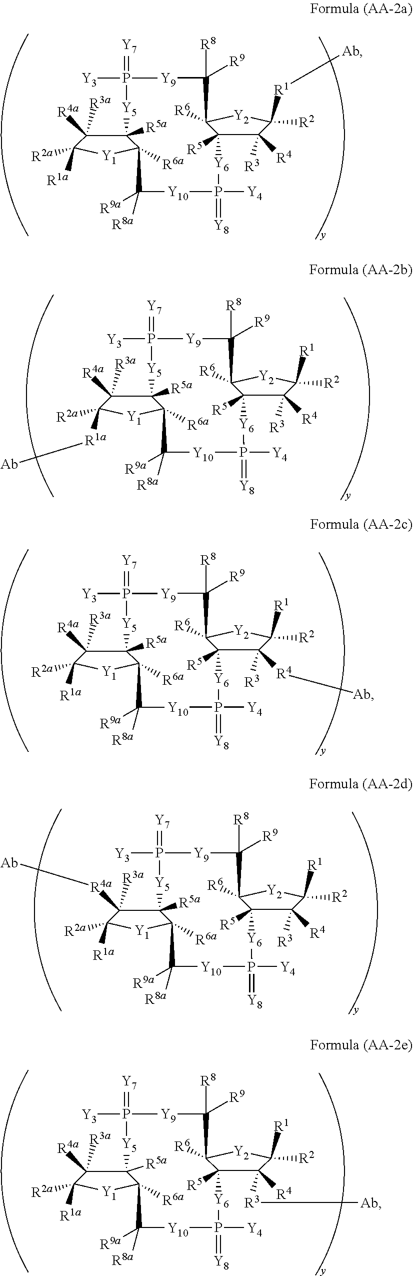

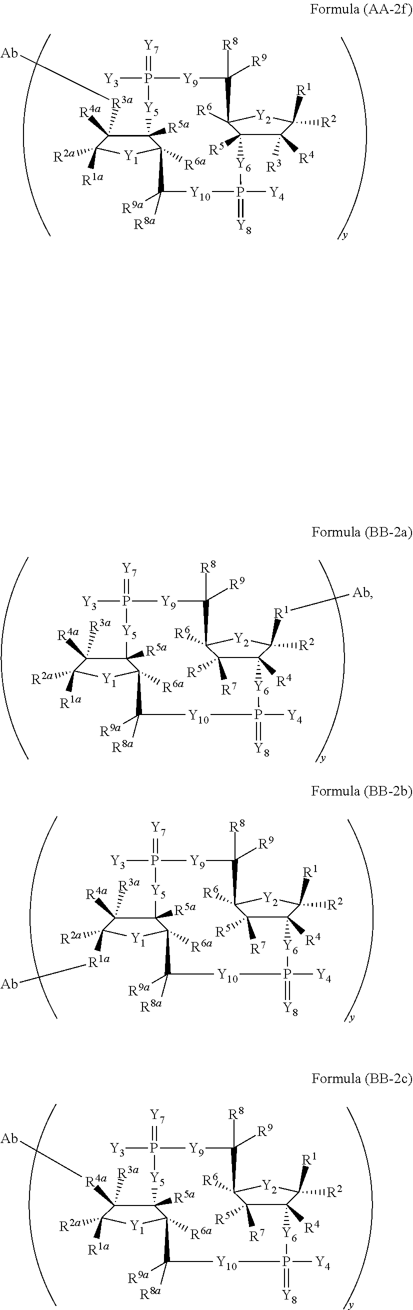

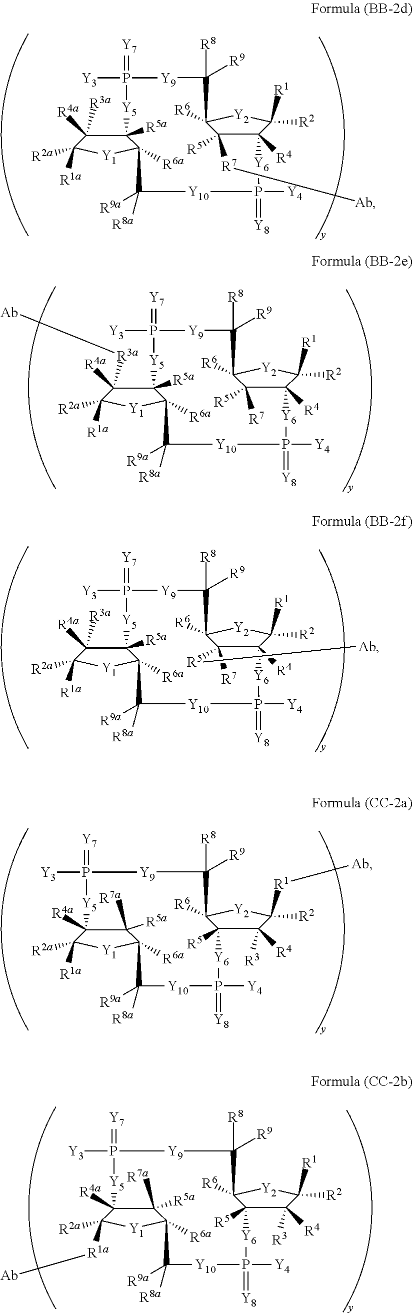

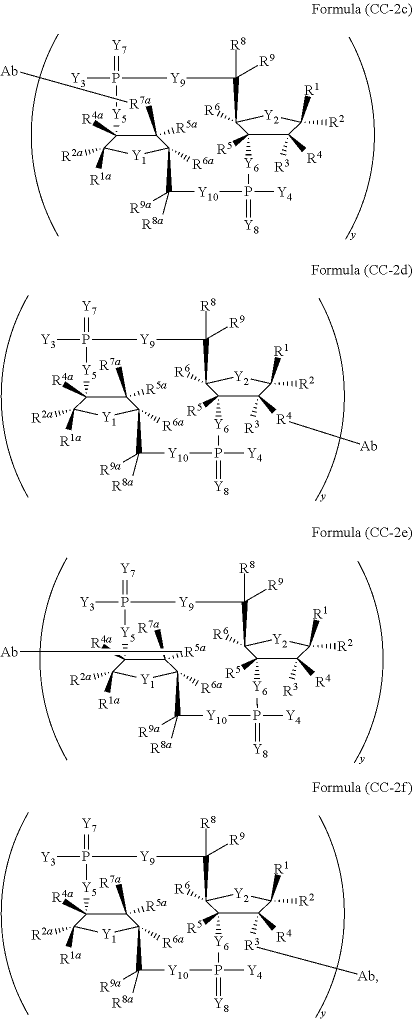

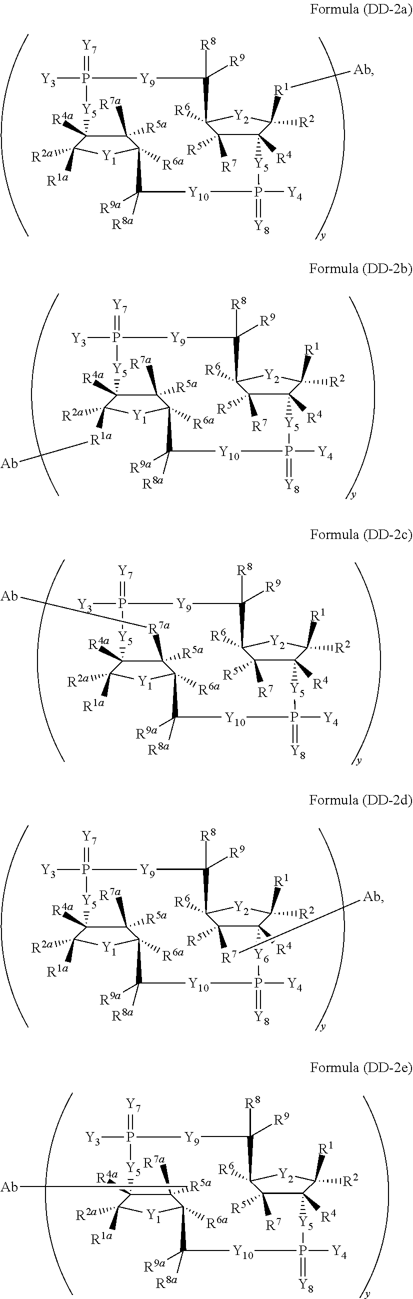

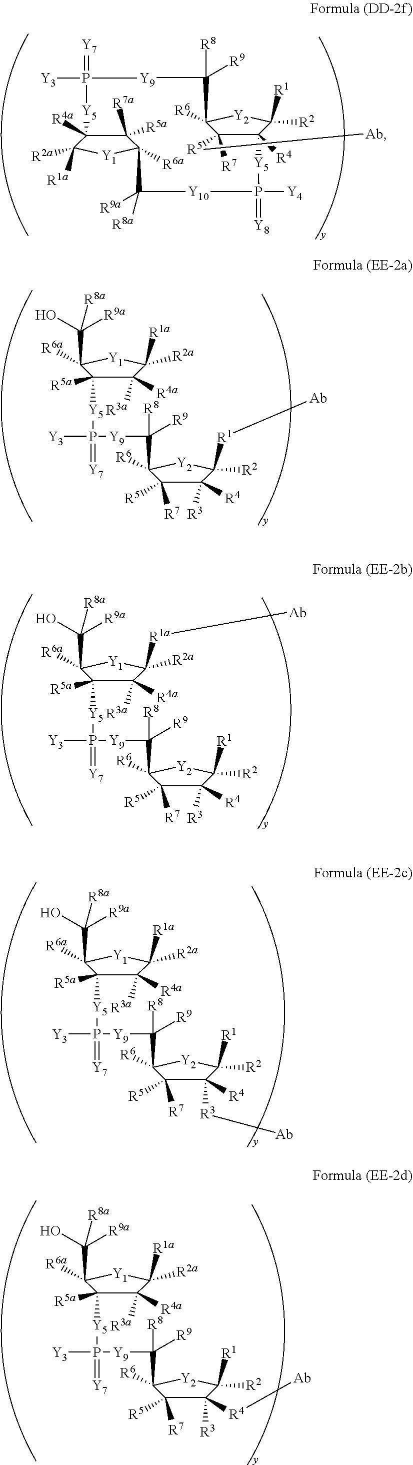

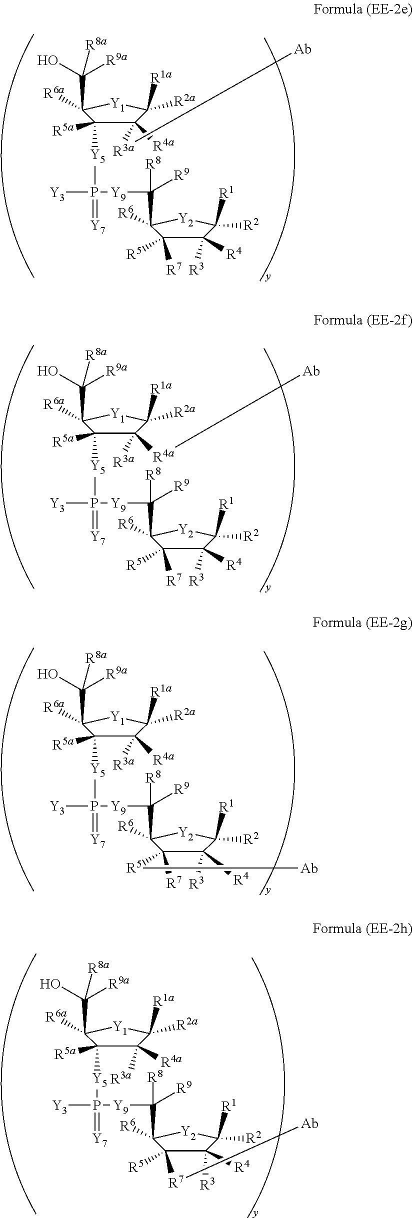

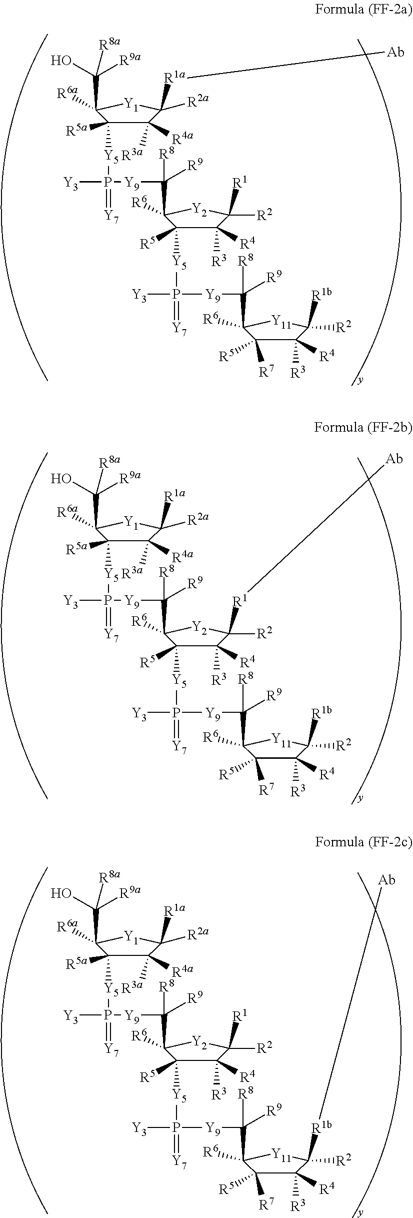

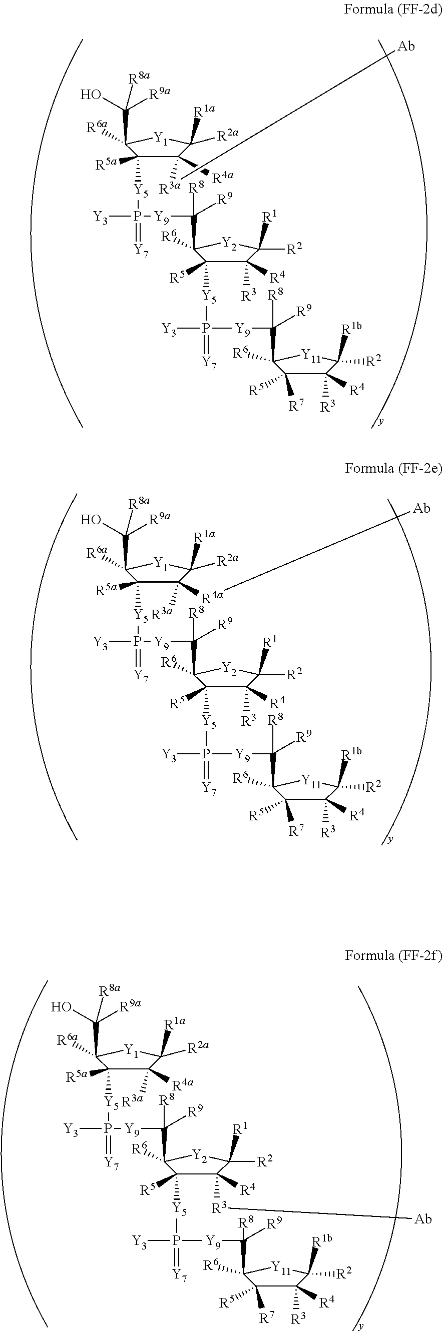

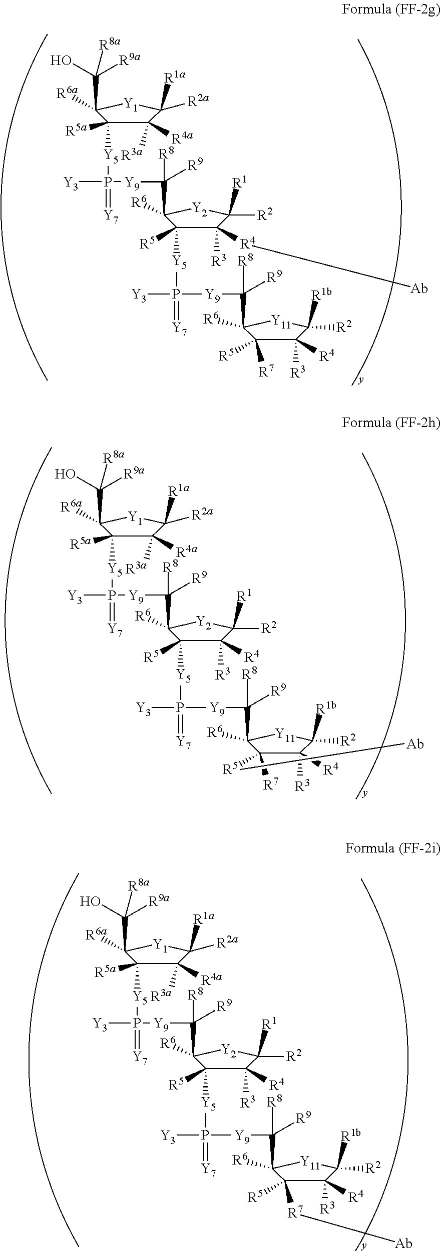

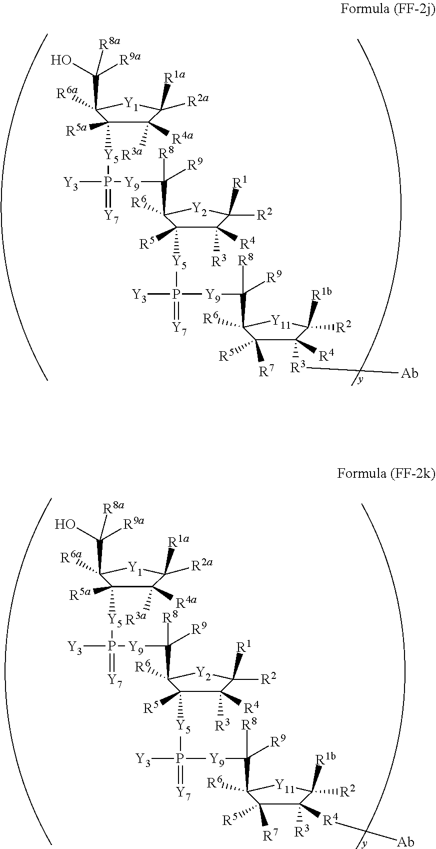

wherein y, Ab, R¹, R^{1a}, R^{1b}, R², R^{2a}, R³, R^{3a}, R⁴, R^{4a}, R⁵, R^{5a}, R⁶, R^{6a}, R⁷, R^{7a}, R⁸, R^{8a}, R⁹, R^{9a}, Y₁, Y₂, Y₃, Y₄, Y₅, Y₆, Y₇, Y₈, Y₉, Y₁₀ and Y₁₁ are as defined above for immunoconjugates of Formulas (AA-a to AA-f), Formulas (BB-a to BB-f), Formulas (CC-a to CC-f), Formulas (DD-a to DD-f), Formulas (EE-a to EE-h) and Formulas (FF-a to FF-k), and provided at least one of R¹, R^{1a} or R^{1b} is substituted with —NHL₁R¹¹⁵, or at least one of R³, R⁴, R⁵, R⁷, R^{3a}, R^{4a}, R^{5a} or R^{7a} is —OL₁R¹¹⁵

### Embodiment 192

The DC-SIGN immunoconjugate of Formula (AA-a to AA-f), Formula (AA-1a to AA-1f) or Formula (AA-2a to AA-2f), wherein
R² and R^{2a} are H;
one of R³ and R⁴ is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ or R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃;
R⁵ and R^{5a} are H;
R⁶ and R^{6a} are H;
R⁸, R⁹, R^{8a} and R^{9a} are independently H or C₁-C₆alkyl, and
one of R^{3a} and R^{4a} is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃, and provided at least one of R¹ or R^{1a} is substituted with —NHL₁R¹¹⁵, or at least one of R³, R⁴, R^{3a} or R^{4a} is —OL₁R¹¹⁵.

### Embodiment 193

The DC-SIGN immunoconjugate of Formula (AA-a to AA-f), Formula (AA-1a to AA-1f) or Formula (AA-2a to AA-2f), wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R⁶, R^{6a}, R⁵ and R^{5a} are H
one of R^{3a} and R^{4a} is H and the other is —OL₁R¹¹⁵, H, OH or F;
one of R³ and R⁴ is H and the other is —OL₁R¹¹⁵, H, OH or F; and
R^{8a}, R^{9a}, R⁸ and R⁹ are independently selected from H or C₁-C₆alkyl,
and provided at least one of R¹ or R^{1a} is substituted with —NHL₁R¹¹⁵, or at least one of R³, R⁴, R^{3a} or R^{4a} is —OL₁R¹¹⁵

### Embodiment 194

The DC-SIGN immunoconjugate of Formula (BB-a to BB-f), Formula (BB-1a to BB-1f) or Formula (BB-2a to BB-2f), wherein:
R² and R^{2a} are H;
one of R^{3a} and R^{4a} is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
R^{5a} and R^{6a} are H;
R⁶ and R⁴ are H;
R⁸, R⁹, R^{8a} and R^{9a} are independently H or C₁-C₆alkyl, and
one of R⁵ and R⁷ is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃,
and provided at least one of R¹ or R^{1a} is substituted with —NHL₁R¹¹⁵, or at least one of R⁵, R⁷, R^{3a} or R^{4a} is —OL₁R¹¹⁵

### Embodiment 195

The DC-SIGN immunoconjugate of Formula (BB-a to BB-f), Formula (BB-1a to BB-1f) or Formula (BB-2a to BB-2f), wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R^{5a}, R^{6a}, R⁶ and R⁴ are H;
one of R^{3a} and R^{4a} is H and the other is —OL₁R¹¹⁵, H, OH or F;
one of R⁵ and R⁷ is H and the other is —OL₁R¹¹⁵, H, OH or F, and
R^{8a}, R^{9a}, R⁸ and R⁹ are independently selected from H or C₁-C₆alkyl,
and provided at least one of R¹ or R^{1a} is substituted with —NHL₁R¹¹⁵, or at least one of R⁵, R⁷, R^{3a} or R^{4a} is —OL₁R¹¹⁵

### Embodiment 196

A DC-SIGN immunoconjugate of Formula (CC-a to CC-f), Formula (CC-1a to CC-1f) or Formula (CC-2a to CC-2f), wherein:
R² and R^{2a} are H;
one of R³ and R⁴ is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃;
R^{4a} and R^{6a} are H;
R⁶ and R⁵ are H;
R⁸, R⁹, R^{8a} and R^{9a} are independently H or C₁-C₆alkyl;
one of R^{5a} and R^{7a} is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} or R^{7a} are substituted by 0, 1, 2 or 3 substituents independently selected from F, Cl, Br, I, OH, CN, and N₃,
and provided at least one of R¹ or R^{1a} is substituted with —NHL₁R¹¹⁵, or at least one of R^{5a}, R^{7a}, R^{3a} or R^{4a} is —OL₁R¹¹⁵

### Embodiment 197

A DC-SIGN immunoconjugate of Formula (CC-a to CC-f), Formula (CC-1a to CC-1f) or Formula (CC-2a to CC-2f), wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₅ and Y₁₀ are O or S;
R², R^{2a}, R^{4a}, R^{6a}, R⁶ and R⁵ are H;
one of R³ and R⁴ is H and the other is —OL₁R¹¹⁵, H, OH or F;
one of R^{5a} and R^{7a} is H and the other is —OL₁R¹¹⁵, H, OH or F, and
R^{8a}, R^{9a}, R⁸ and R⁹ are independently selected from H or C₁-C₆alkyl,
and provided at least one of R¹ or R^{1a} is substituted with —NHL₁R¹¹⁵, or at least one of R^{5a}, R^{7a}, R^{3a} or R^{4a} is —OL₁R¹¹⁵

### Embodiment 198

A DC-SIGN immunoconjugate of Formula (DD-a to DD-f), Formula (DD-1a to DD-1f) or Formula (DD-2a to DD-2f), wherein:
R² and R^{2a} are H;
one of R^{5a} and R^{7a} is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} or R^{7a} are substituted by 0, 1, 2 or 3 substituents independently selected from F, Cl, Br, I, OH, CN, and N₃;
R^{4a} and R^{6a} are H;
R⁶ and R⁴ are H;
R⁸, R⁹, R^{8a} and R^{9a} are independently H or C₁-C₆alkyl, and
one of R⁵ and R⁷ is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃,
and provided at least one of R¹ or R^{1a} is substituted with —NHL₁R¹¹⁵, or at least one of R^{5a}, R^{7a}, R⁵ or R⁷ is —OL₁R¹¹⁵

### Embodiment 199

A DC-SIGN immunoconjugate of Formula (DD-a to DD-f), Formula (DD-1a to DD-1f) or Formula (DD-2a to DD-2f), wherein:
Y₁ and Y₂ are O, CH₂ or S;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₅ and Y₆ are O or S;
Y₇ and Y₈ are O or S;
Y₉ and Y₁₀ are O or S;
R², R^{2a}, R^{4a}, R^{6a}, R⁶ and R⁴ are H;
one of R^{5a} and R^{7a} is H and the other is —OL₁R¹¹⁵, OH or F;
one of R⁵ and R⁷ is H and the other is —OL₁R¹¹⁵, H, OH or F, and
R⁸, R⁹, R^{8a} and R^{9a} are independently H or C₁-C₆alkyl,
and provided at least one of R¹ or R^{1a} is substituted with —NHL₁R¹¹⁵, or at least one of R^{5a}, R^{7a}, R⁵ or R⁷ is —OL₁R¹¹⁵

### Embodiment 200

A DC-SIGN immunoconjugate of Formula (EE-a to EE-h), Formula (EE-1a to EE-1h) or Formula (EE-2a to EE-2h), wherein: R² and R^{2a} are H;
R⁶ and R^{6a} are H;
R⁷ is H;
R⁸, R⁹, R^{8a} and R^{9a} are independently H or C₁-C₆alkyl, and
one of R^{3a} and R^{4a} is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
one of R³ and R⁴ is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃, and
one of R⁵ and R⁷ is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ or R⁷ are substituted by 0, 1, 2 or 3 substituents independently selected from F, Cl, Br, I, OH, CN, and N₃,
and provided at least one of R¹ or R^{1a} is substituted with —NHL₁R¹¹⁵, or at least one of R^{3a}, R^{4a}, R³, R⁴, R⁵ or R⁷ is —OL₁R¹¹⁵.

### Embodiment 201

A DC-SIGN immunoconjugate of Formula (EE-a to EE-h), Formula (EE-1a to EE-1h) or Formula (EE-2a to EE-2h), wherein:
Y₁ and Y₂ are O, CH₂ or S;
Y₃ is OH, O⁻, OR¹⁰, N(R¹⁰)₂, SH or S⁻;
Y⁵ is O or S;
Y₇ is O or S;
Y₉ is O or S;
R², R^{2a}, R⁵, R^{6a}, R⁶ and R⁷ are H;
one of R^{3a}, R^{4a} is H and the other is —OL₁R¹¹⁵, H, OH, OCH₃ or F;
one of R³, R⁴ is H and the other is —OL₁R¹¹⁵, H, OH, OCH₃ or F;
one of R⁵ and R⁷ is H and the other is —OL₁R¹¹⁵, H, OH, OCH₃ or F, and
R⁸, R⁹, R^{8a} and R^{9a} are independently H or C₁-C₆alkyl,
and provided at least one of R¹ or R^{1a} is substituted with —NHL₁R¹¹⁵, or at least one of R^{3a}, R^{4a}, R³, R⁴, R⁵ or R⁷ is —OL₁R¹¹⁵

### Embodiment 202

A DC-SIGN immunoconjugate of Formula (FF-a to FF-k), Formula (FF-1a to FF-1 k) or Formula (FF-2a to FF-2k), wherein:
R² and R^{2a} are H;
each R⁶ and R^{6a} are H;
R^{5a} and R⁷ are H;
R⁸, R⁹, R^{8a} and R^{9a} are independently H or C₁-C₆alkyl, and
one of R^{3a} and R^{4a} is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} or R^{4a} are substituted by 0, 1, 2 or 3 substituents independently selected from F, Cl, Br, I, OH, CN, and N₃;
one of R³ and R⁴ is H and the other is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ or R⁴ are substituted by 0, 1, 2 or 3 substituents independently selected from F, Cl, Br, I, OH, CN, and N₃, and
R⁵ is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ is substituted by 0, 1, 2 or 3 substituents independently selected from F, Cl, Br, I, OH, CN, and N₃,
and provided at least one of R¹, R^{1a} or R^{1b} is substituted with —NHL₁R¹¹⁵, or at least one of R^{3a}, R^{4a}, R³, R⁴, R⁵ or R⁷ is —OL₁R¹¹⁵

### Embodiment 203

A DC-SIGN immunoconjugate of Formula (FF-a to FF-k), Formula (FF-1a to FF-1 k) or Formula (FF-2a to FF-2k), wherein:
Y₁ and Y₂ are O, CH₂ or S;
each Y₃ is independently OH, O⁻, OR¹⁰, N(R¹⁰)₂, SH or S⁻;
each Y⁵ is independently O or S;
each Y₇ is independently O or S;
each Y₉ is independently O or S;
Y₁₁ is O, CH₂ or S;
R², R^{2a}, R⁶, R^{6a}, R^{5a}, and R^{7a} are H;
one of R^{3a} and R^{4a} is H and the other is —OL₁R¹¹⁵, H, OH, OCH₃ or F;
one of R³ and R⁴ is H and the other is —OL₁R¹¹⁵, H, OH, OCH₃ or F;
one of R⁵ and R⁷ is H and the other is —OL₁R¹¹⁵, H, OH, OCH₃ or F, and
R⁸, R⁹, R^{8a} and R^{9a} are independently H or C₁-C₆alkyl,
and provided at least one of R¹, R^{1a} or R^{1b} is substituted with —NHL₁R¹¹⁵, or at least one of R^{3a}, R^{4a}, R³, R⁴, R⁵ or R⁷ is —OL₁R¹¹⁵

### Embodiment 204

A DC-SIGN immunoconjugate of Formula (AA-a to AA-f), Formula (BB-a to BB-f), Formula (CC-a to CC-f), Formula (DD-a to DD-f), Formula (EE-a to EE-h), Formula (FF-a to FF-k) or an immunoconjugate of any one of Embodiments 146 to 161, wherein:
R¹ is

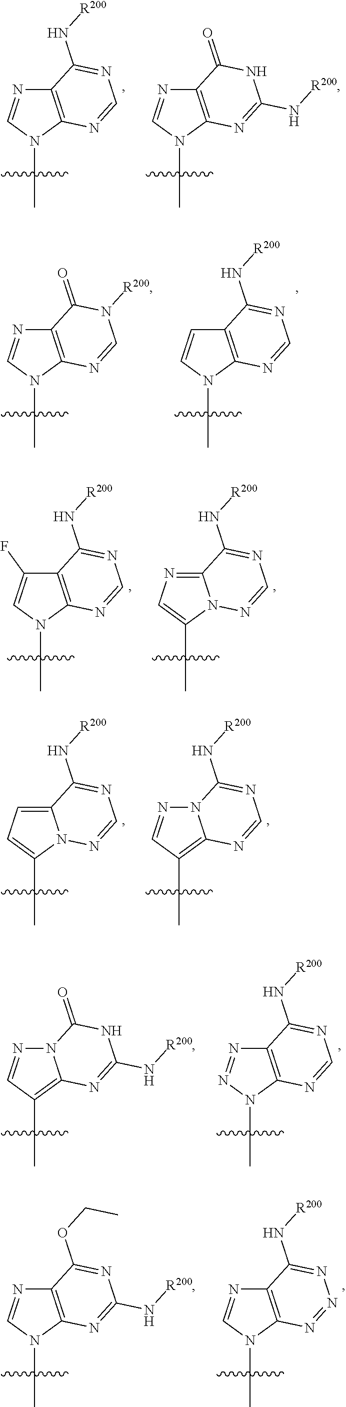

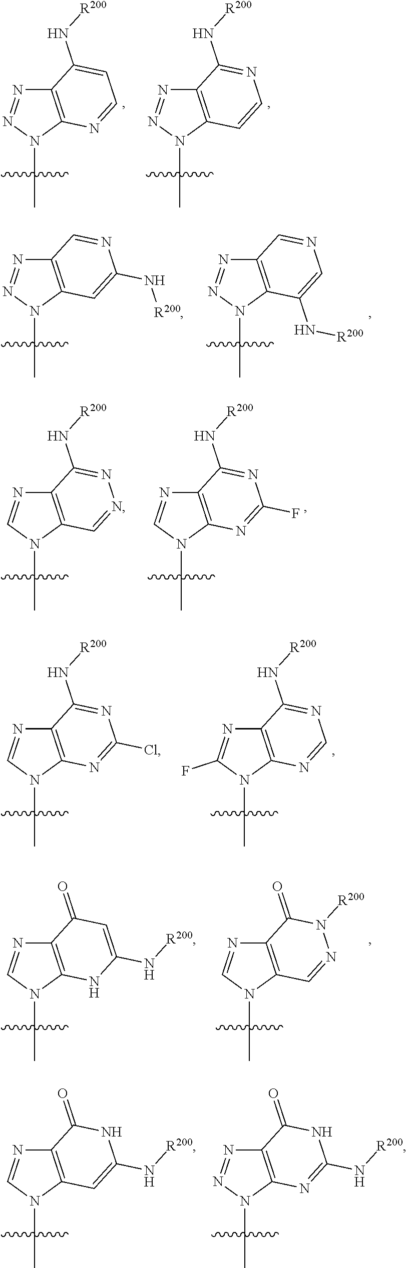

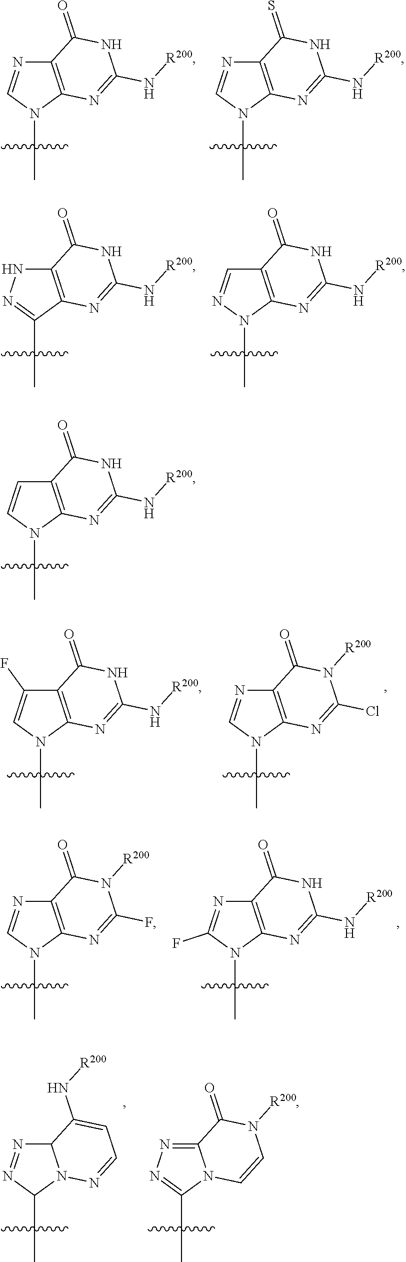

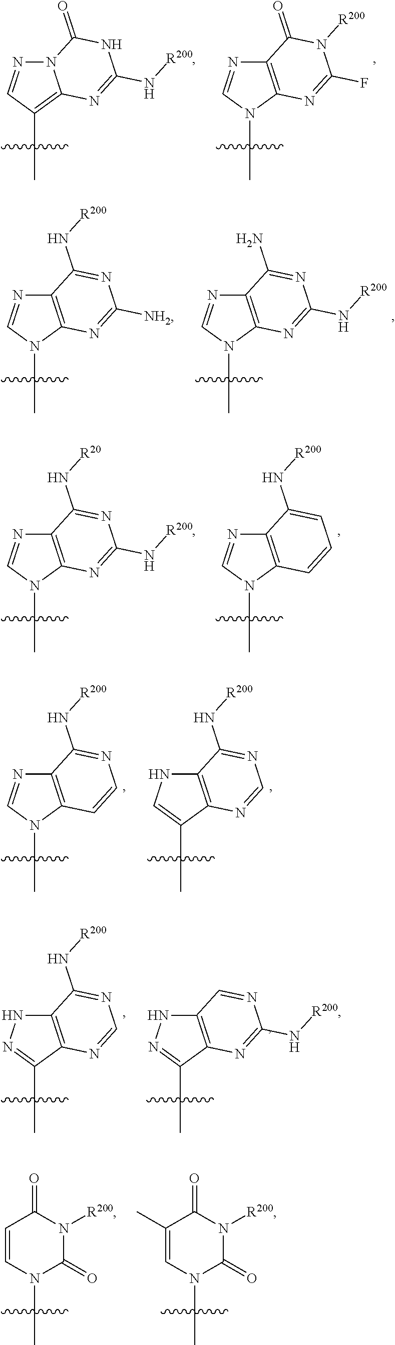

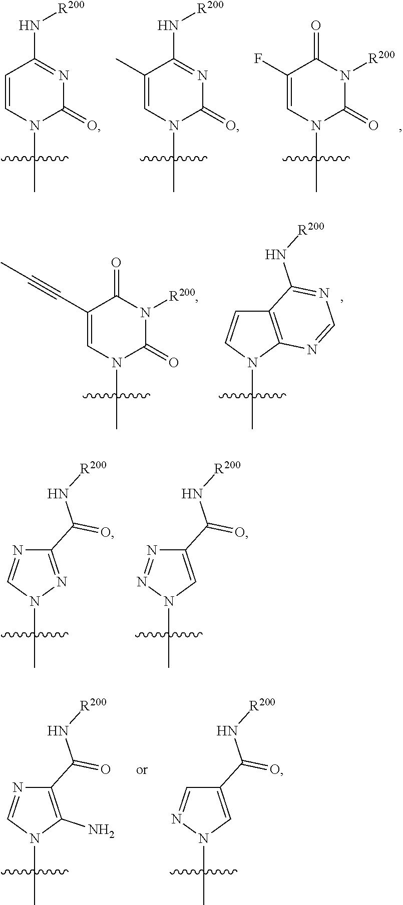

wherein: R¹ is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
   and
   each R²⁰⁰ is independently selected from H and L₁R¹¹⁵;
R^{1a} is

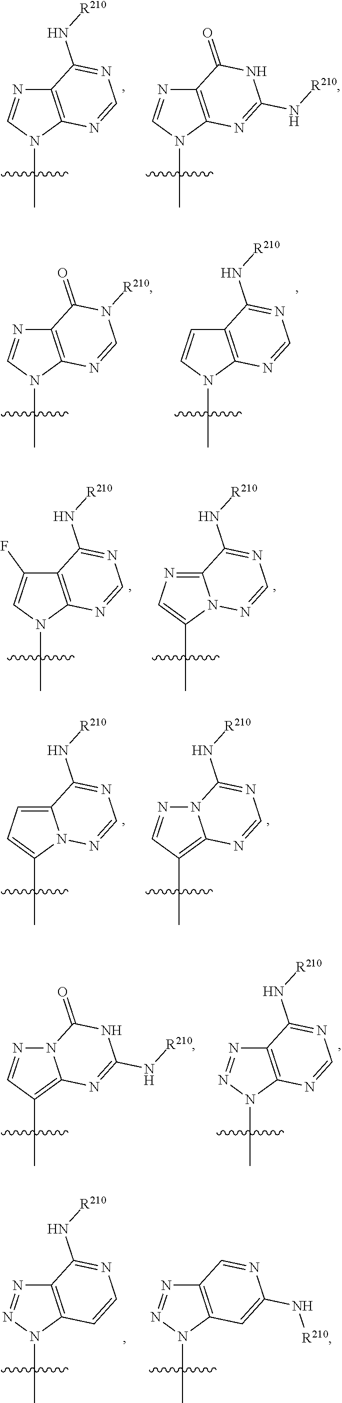

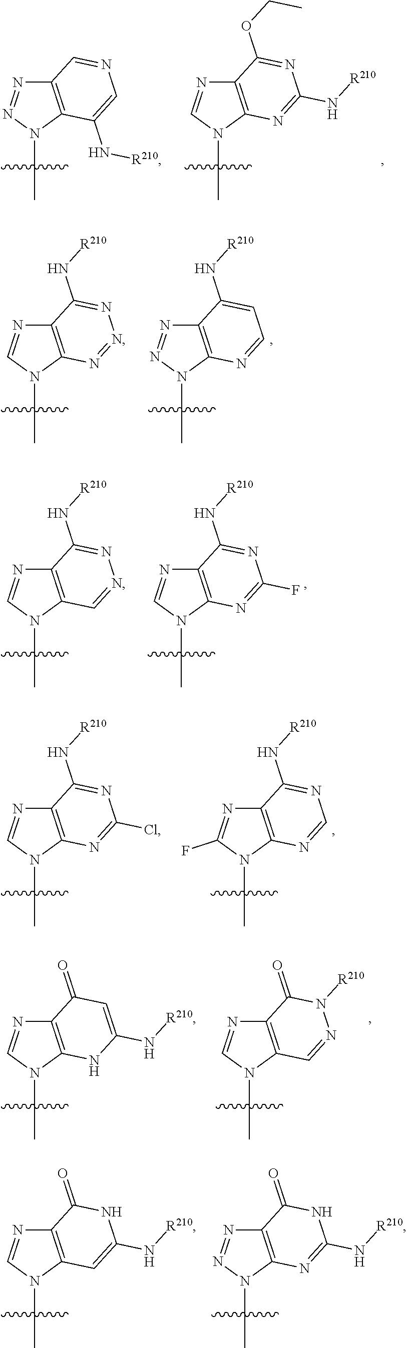

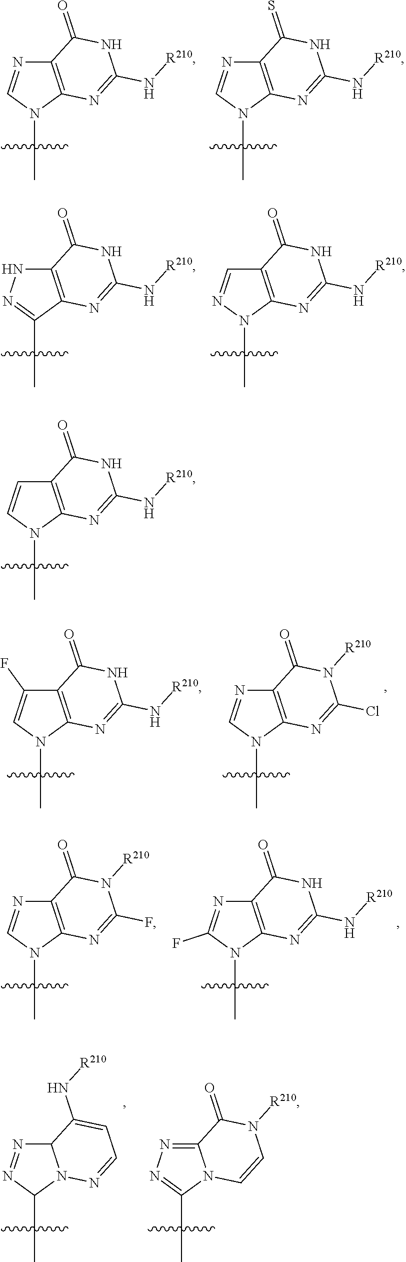

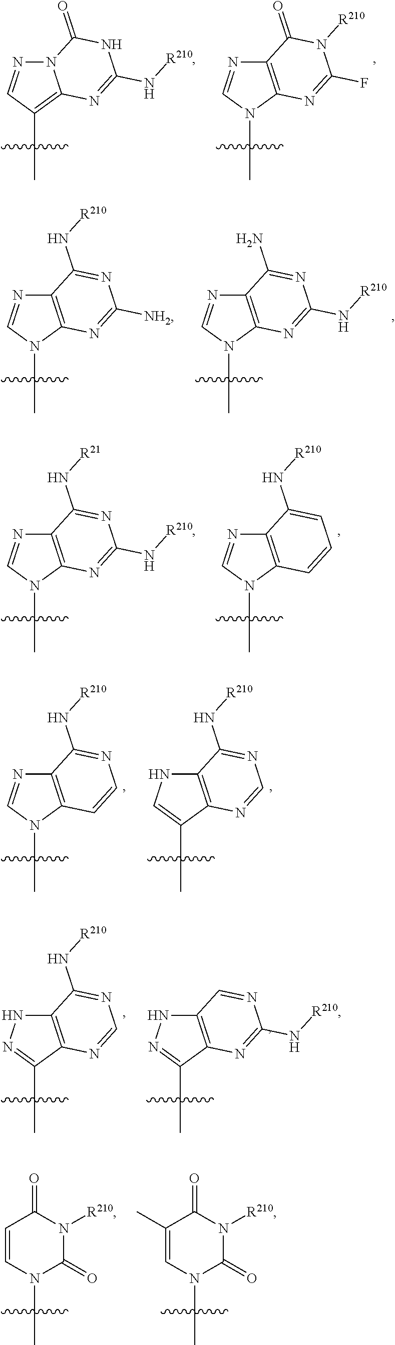

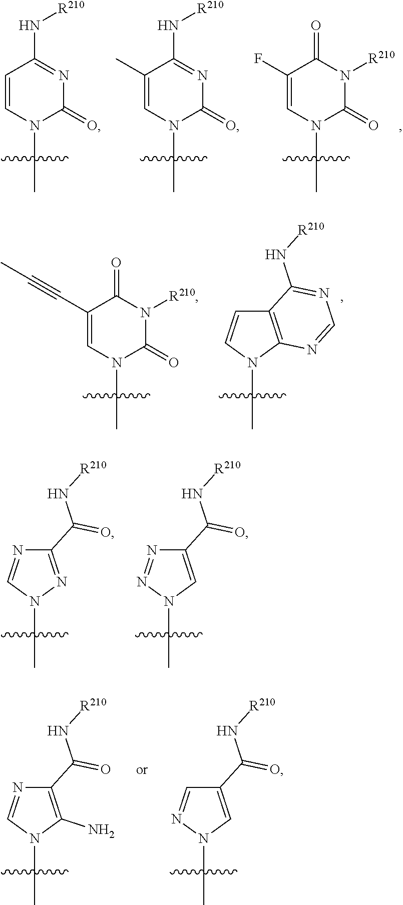

wherein: R^{1a} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —ON, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
   and
   each R²¹⁰ is independently selected from H and L₁R¹¹⁵,
and
R^{1b} is

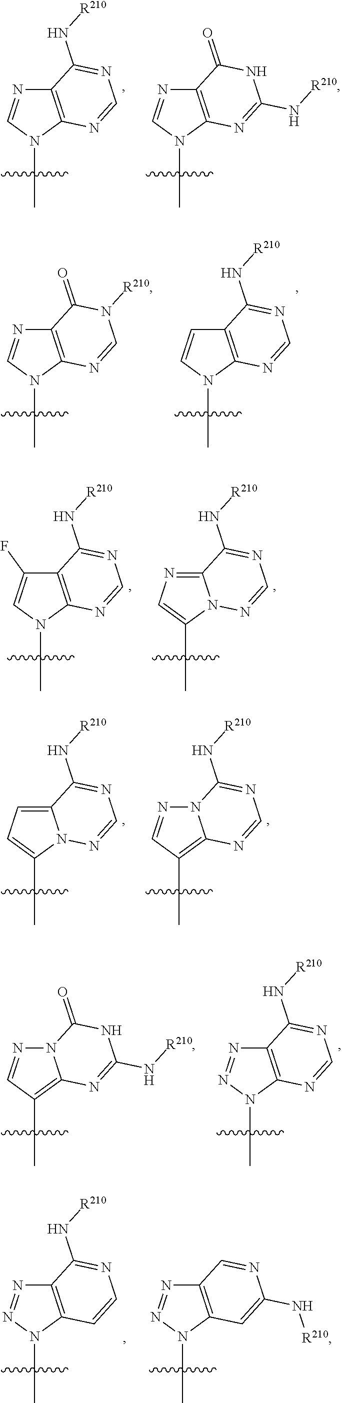

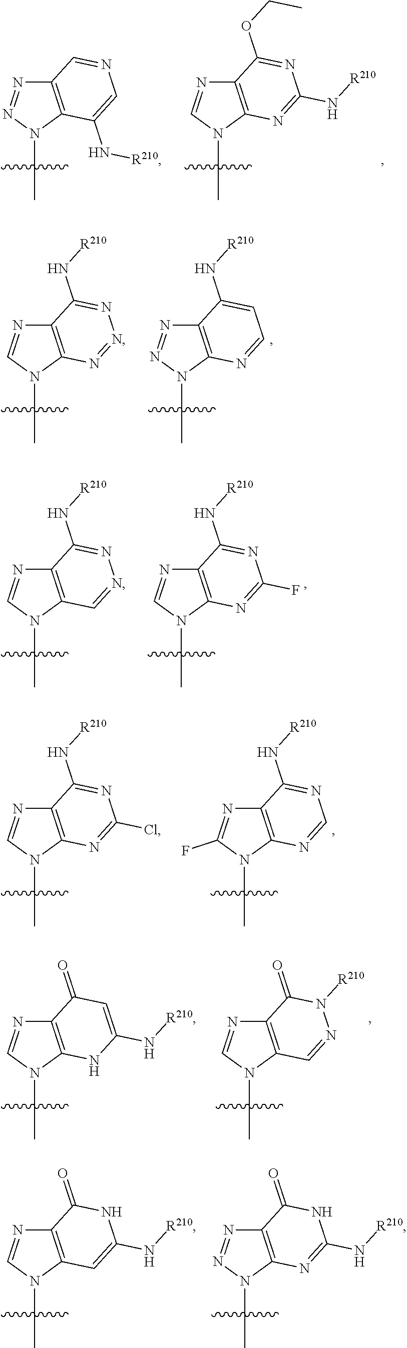

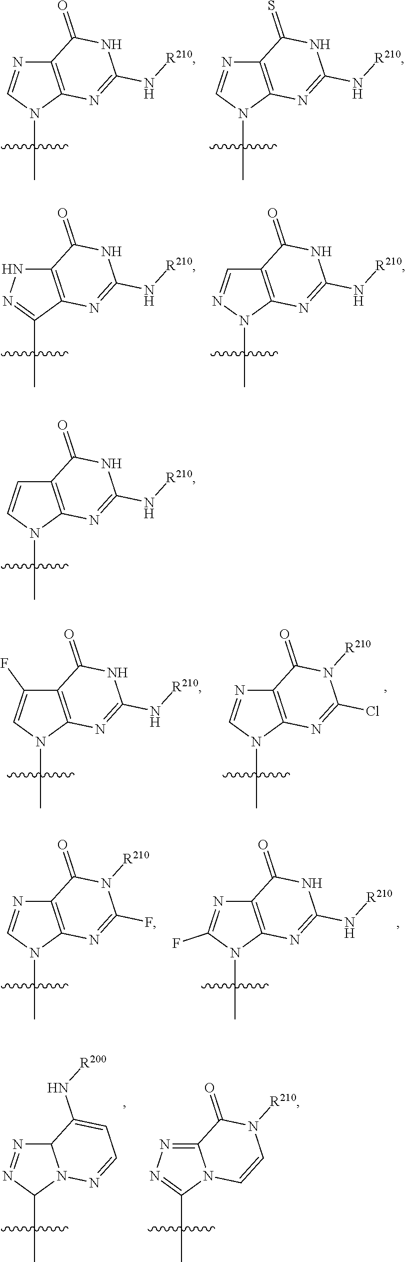

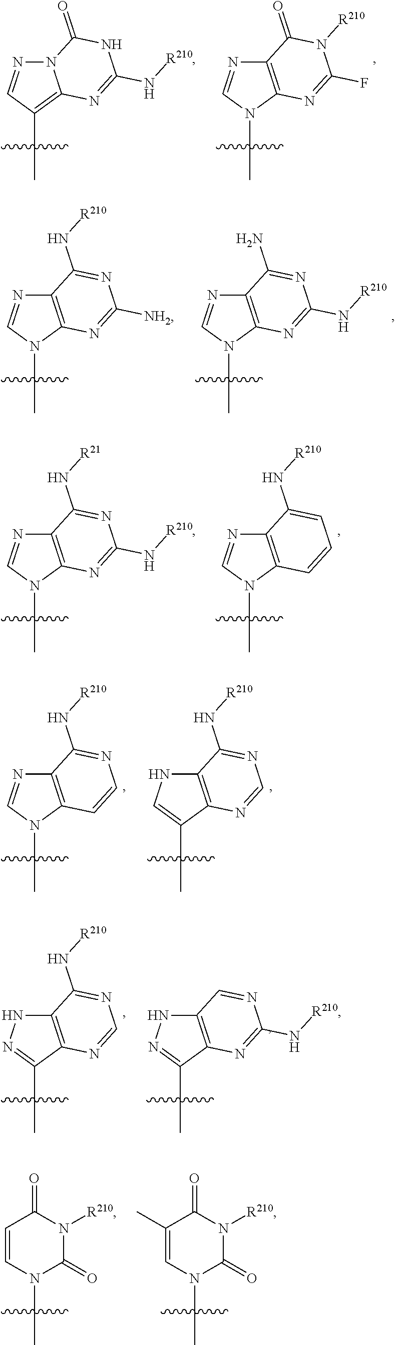

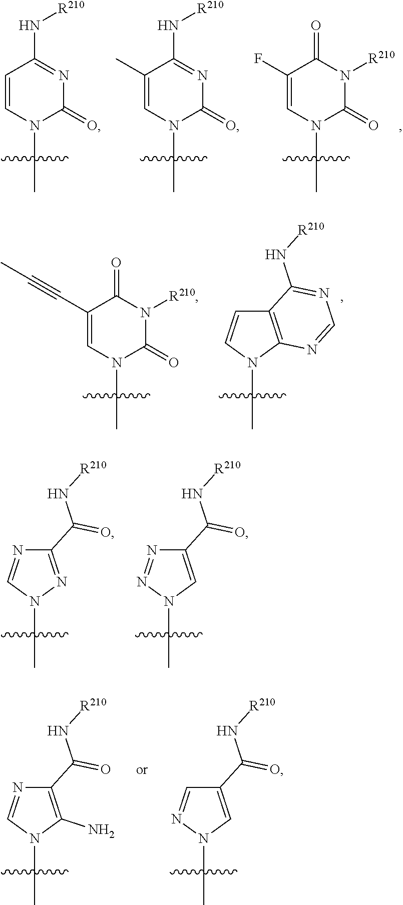

wherein: R^{1b} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
   and
   each R²¹⁰ is independently selected from H and L₁R¹¹⁵

### Embodiment 205

A DC-SIGN immunoconjugate selected from:

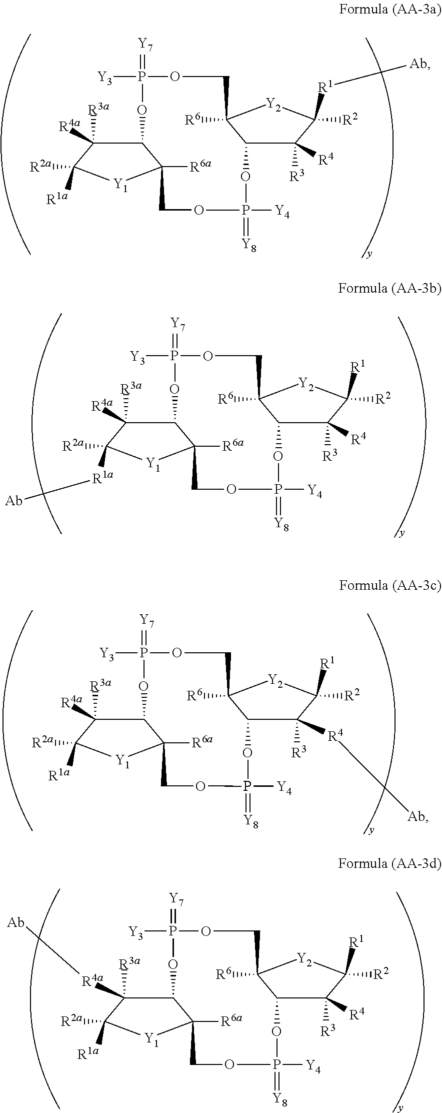

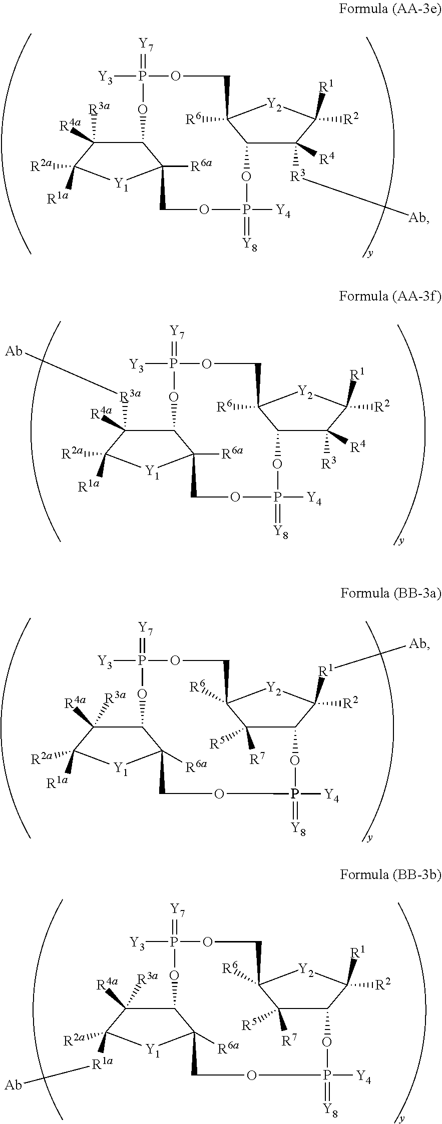

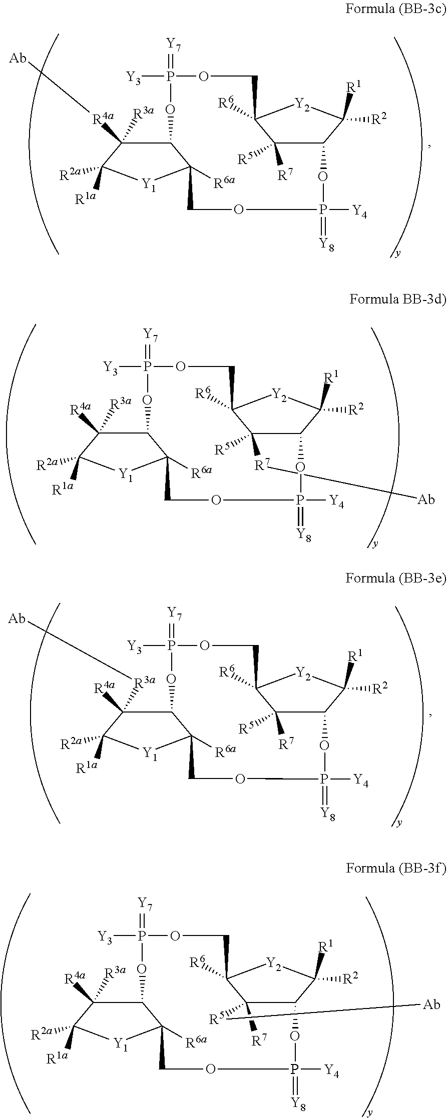

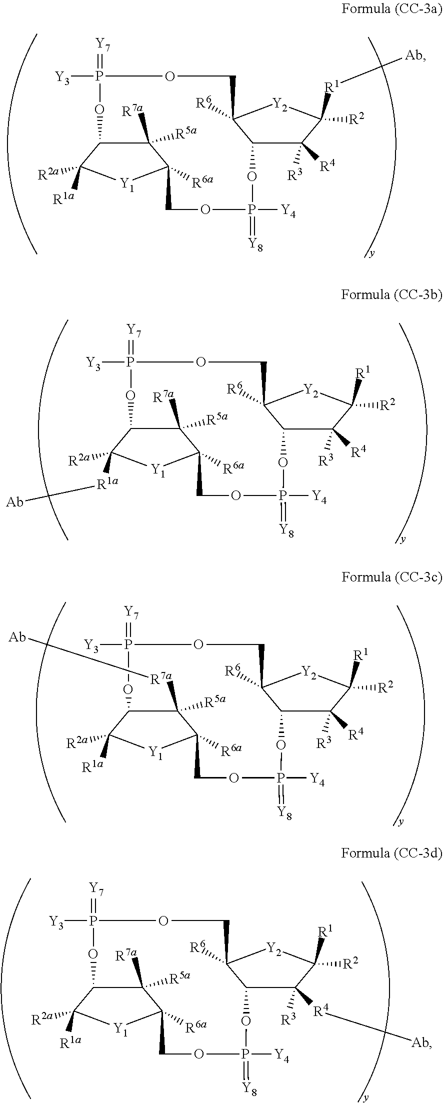

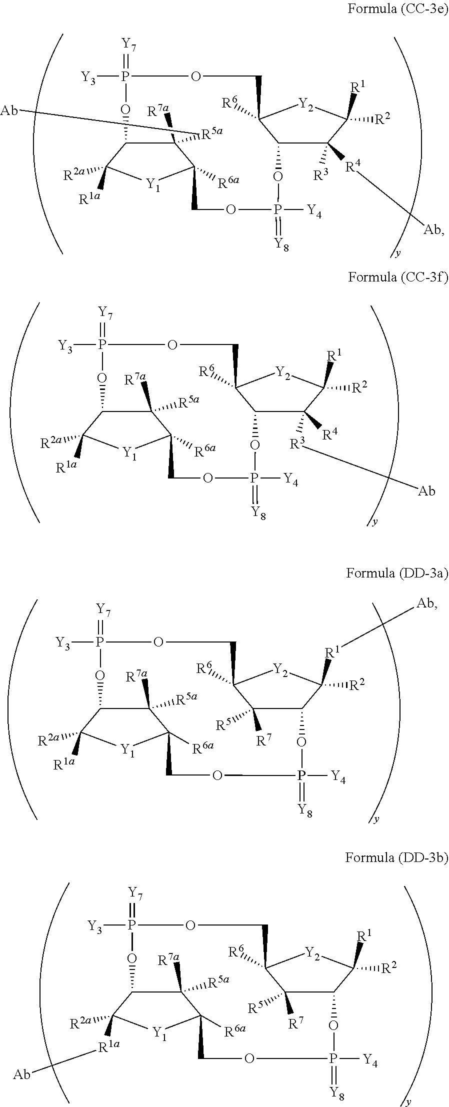

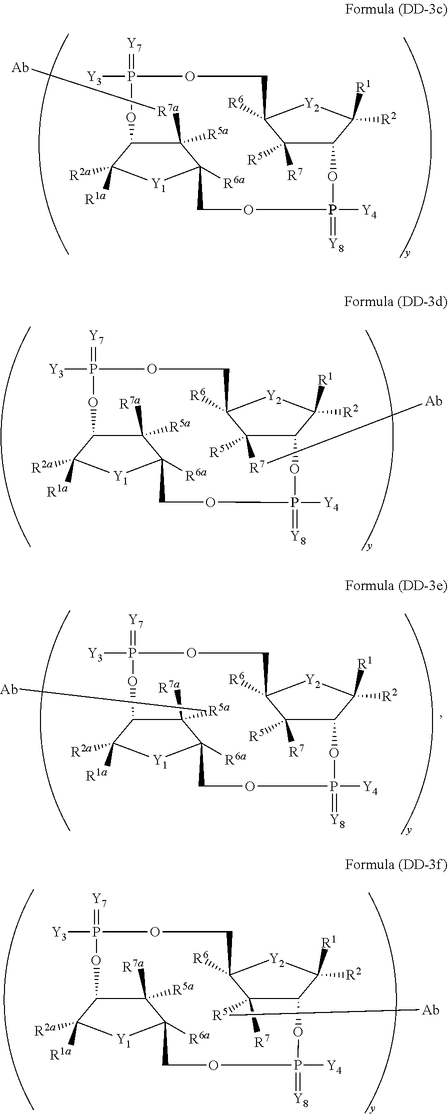

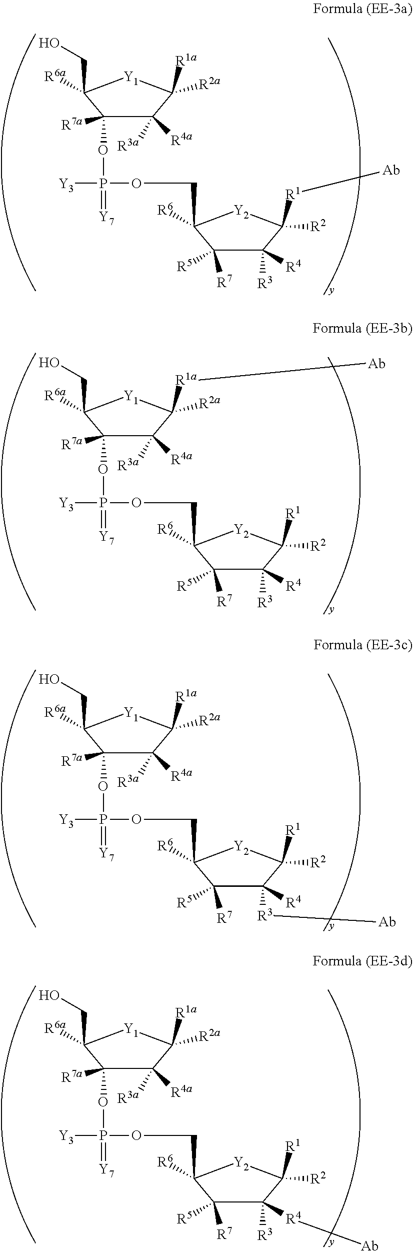

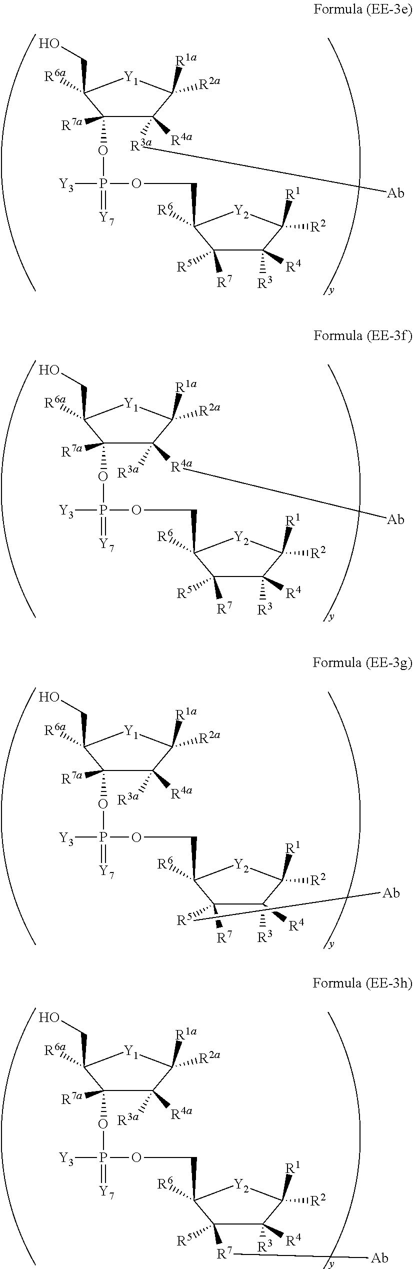

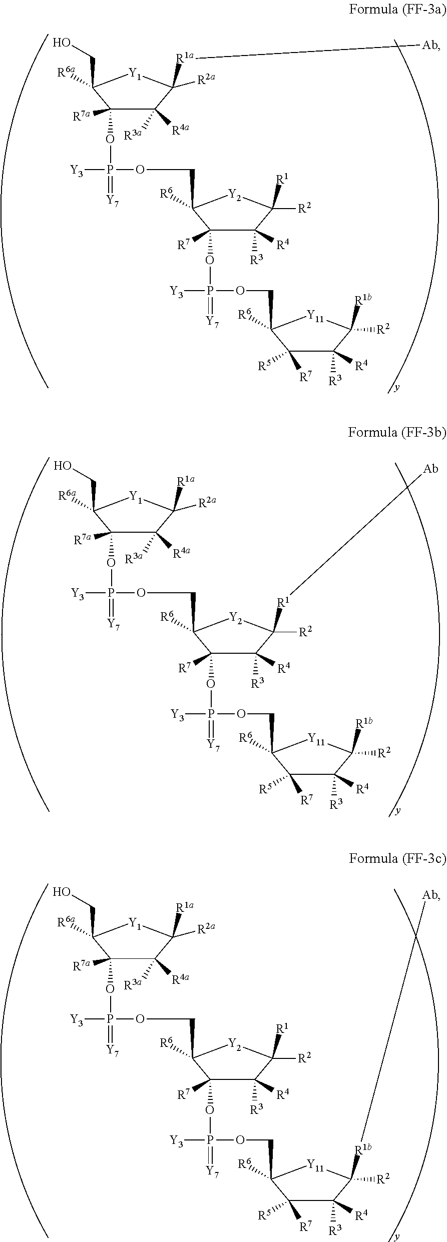

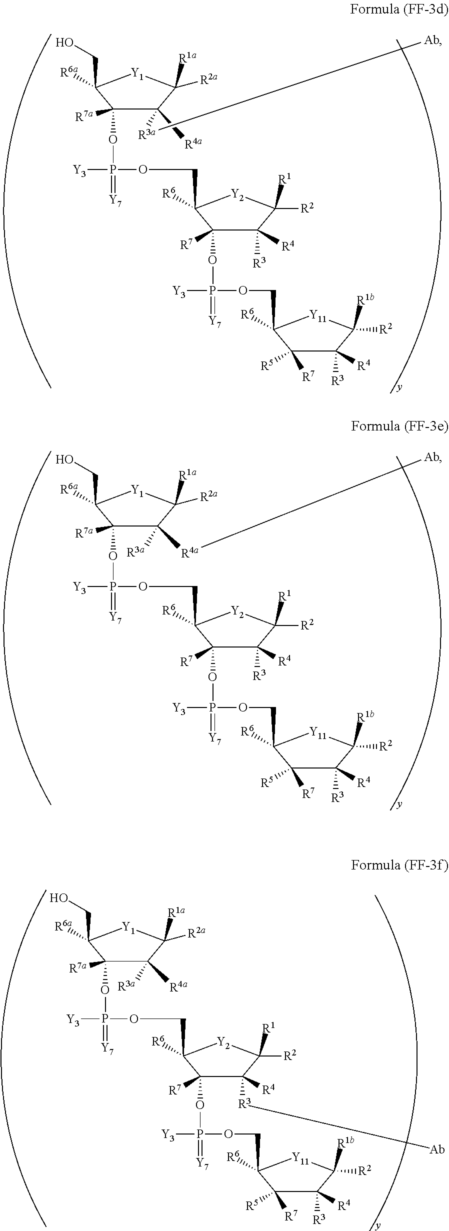

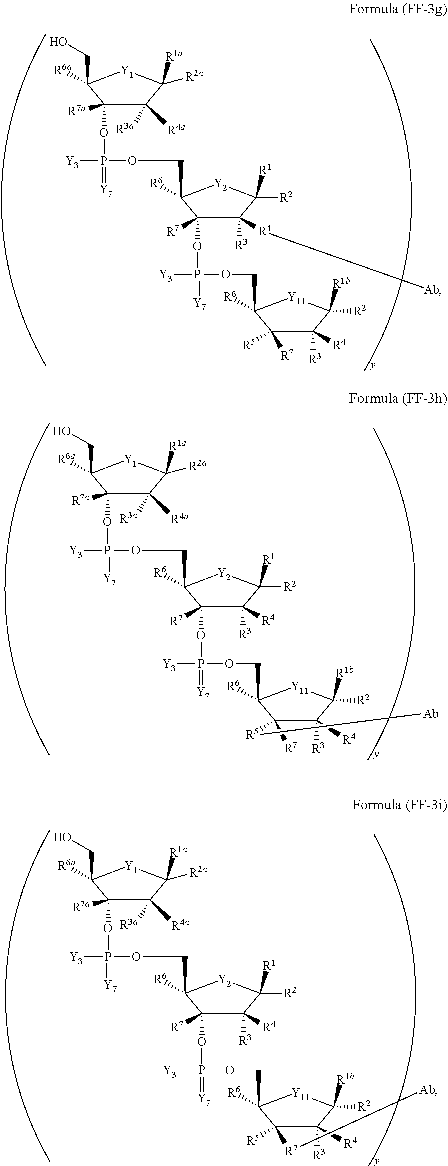

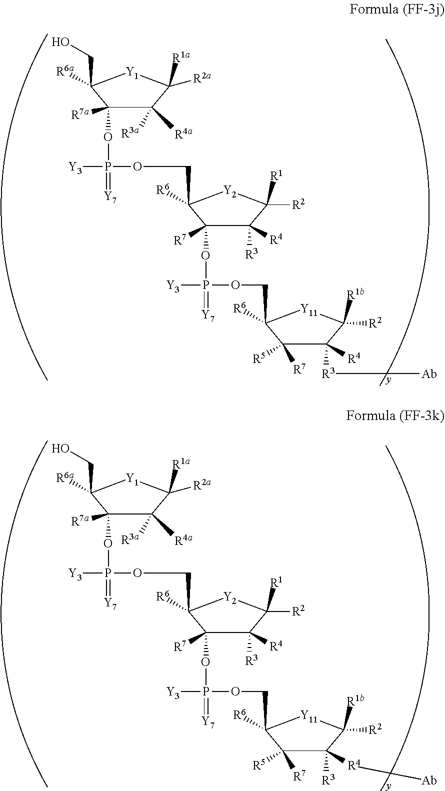

wherein:
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₃ is OH, O⁻, OR¹⁰, N(R¹⁰)₂, SH or S⁻;
Y₄ is OH, O⁻, OR¹⁰, N(R¹⁰)₂, SH or S⁻;
Y₇ is O or S;
Y₈ is O or S;
Y₁₁ is —O—, —S—, —S(═O)—, —SO₂—, —CH₂—, or —CF₂—;
R¹ is

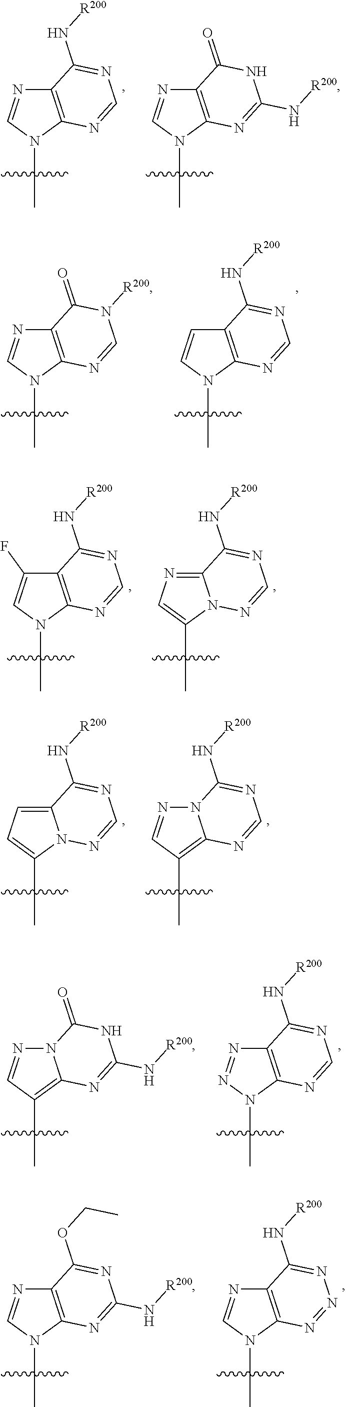

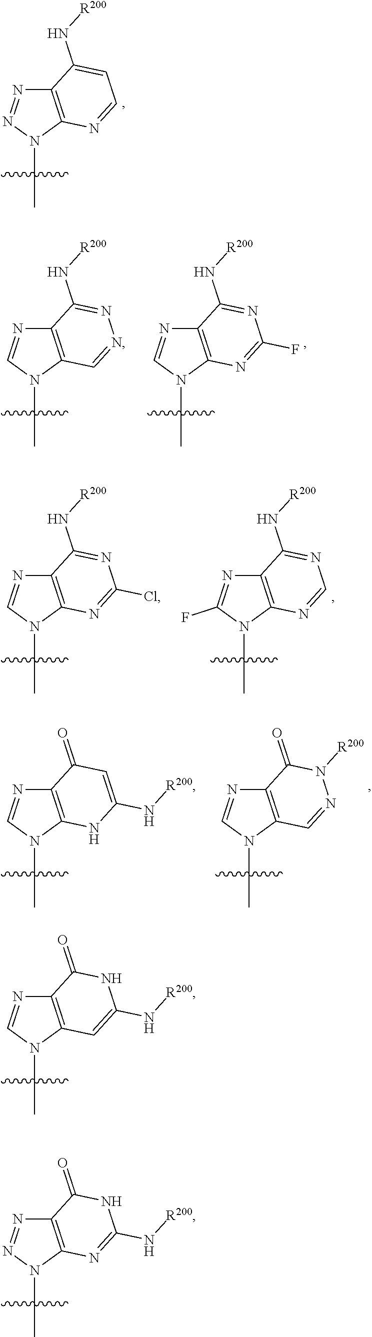

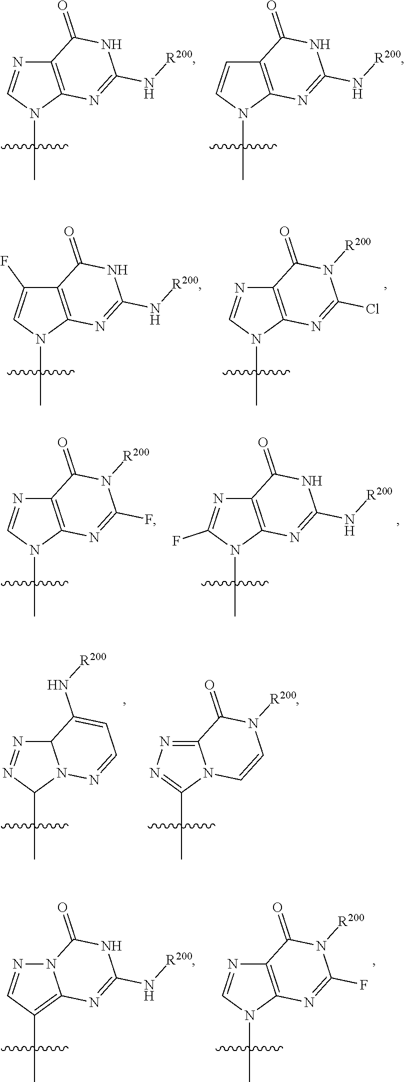

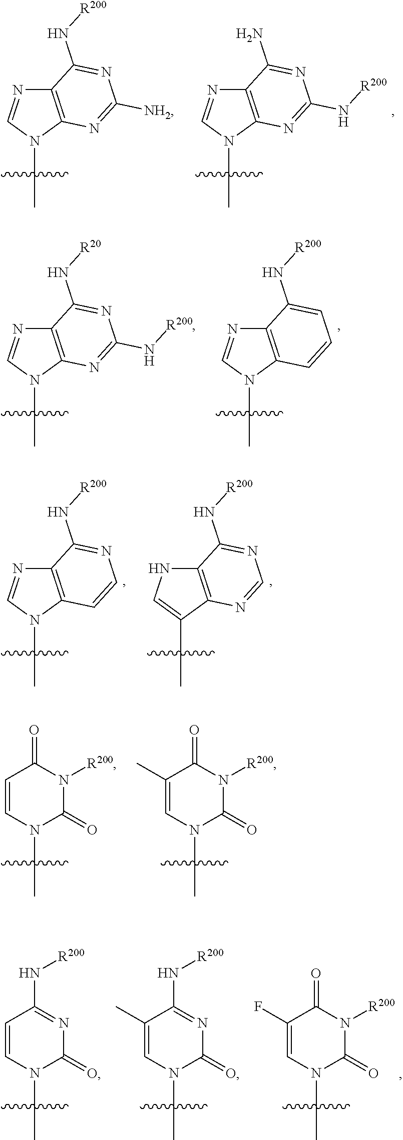

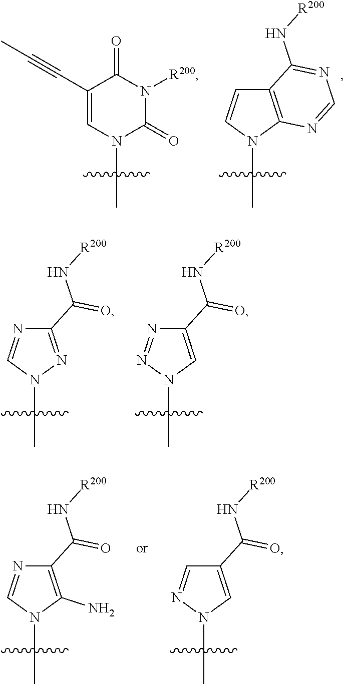

wherein: R¹ is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²⁰⁰ is independently selected from H and L₁R¹¹⁵
R^{1a} is

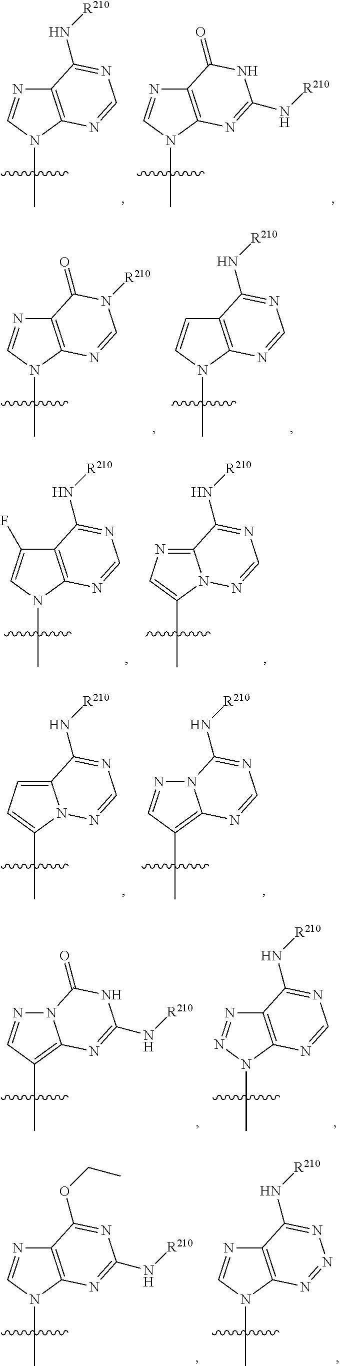

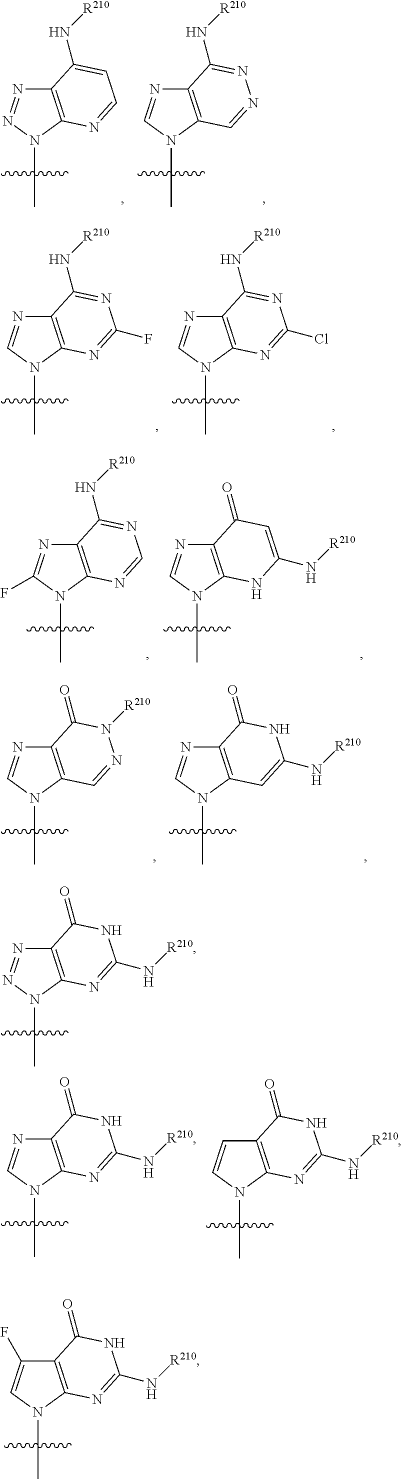

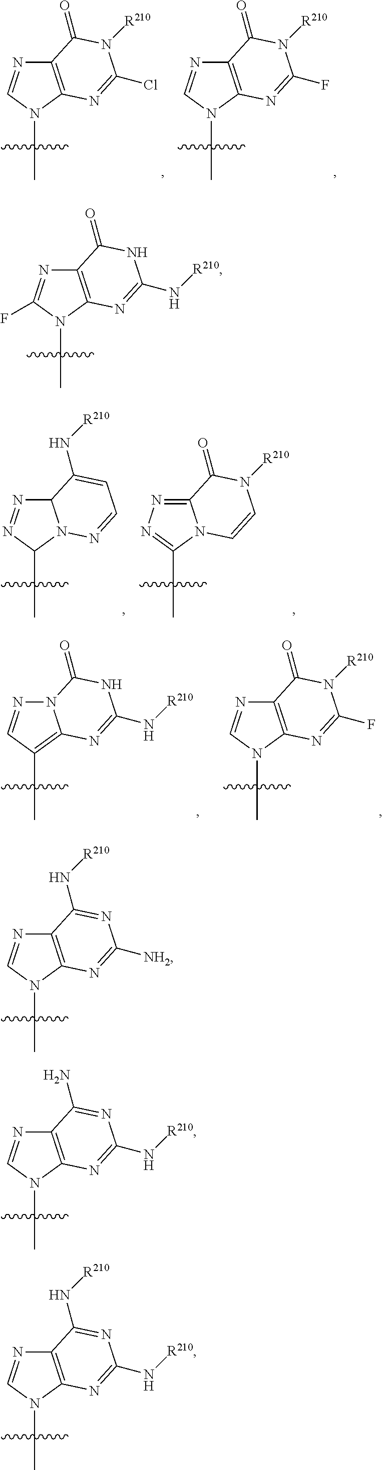

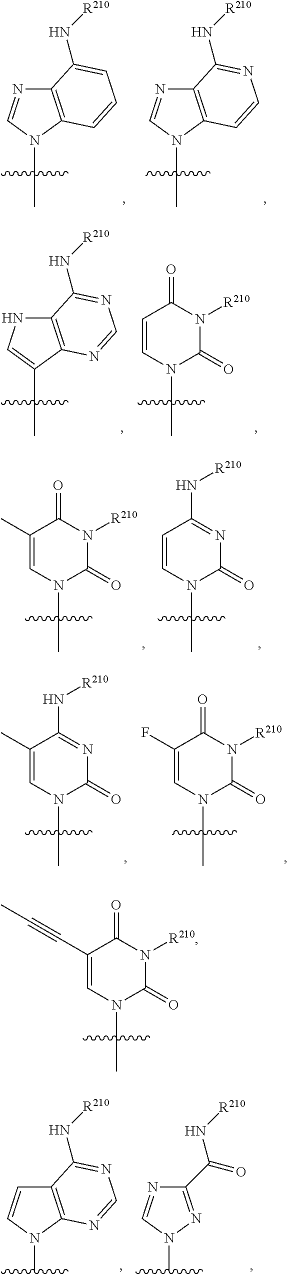

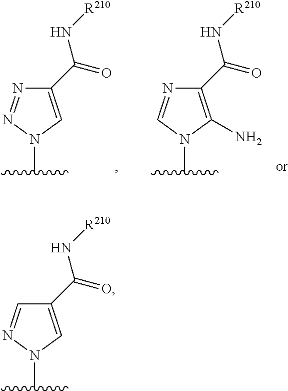

wherein: R^{1a} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²¹⁰ is independently selected from H and L₁R¹¹⁵,
R^{1b} is

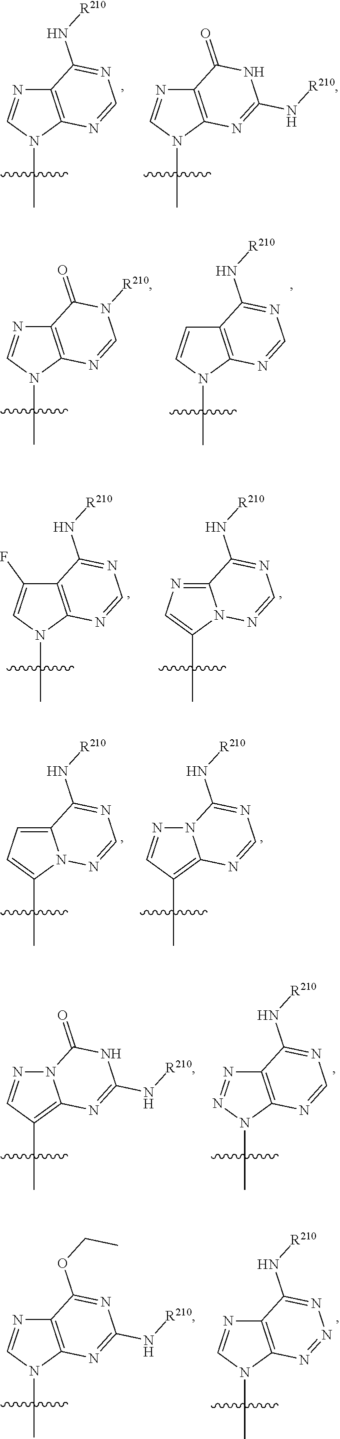

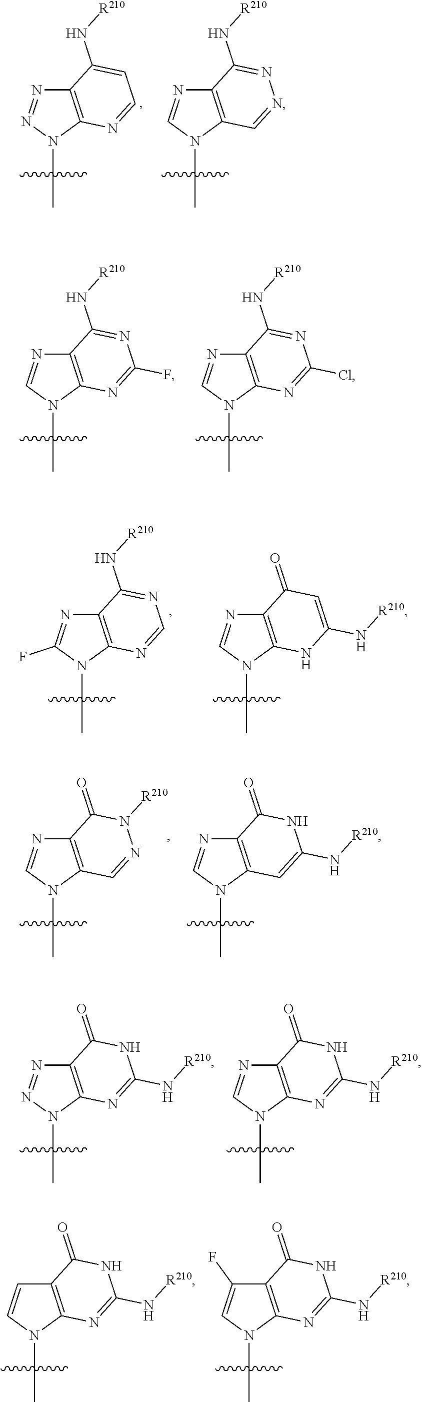

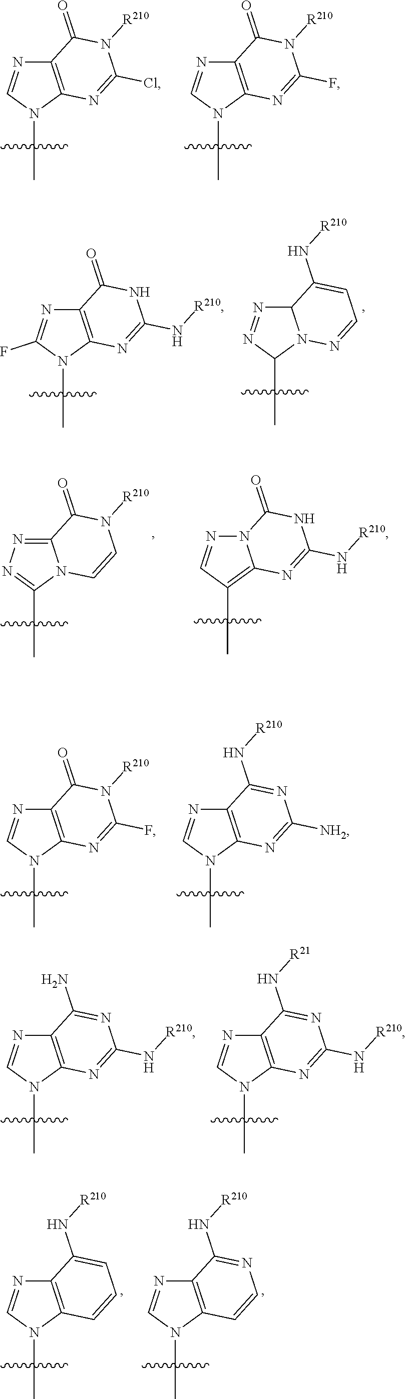

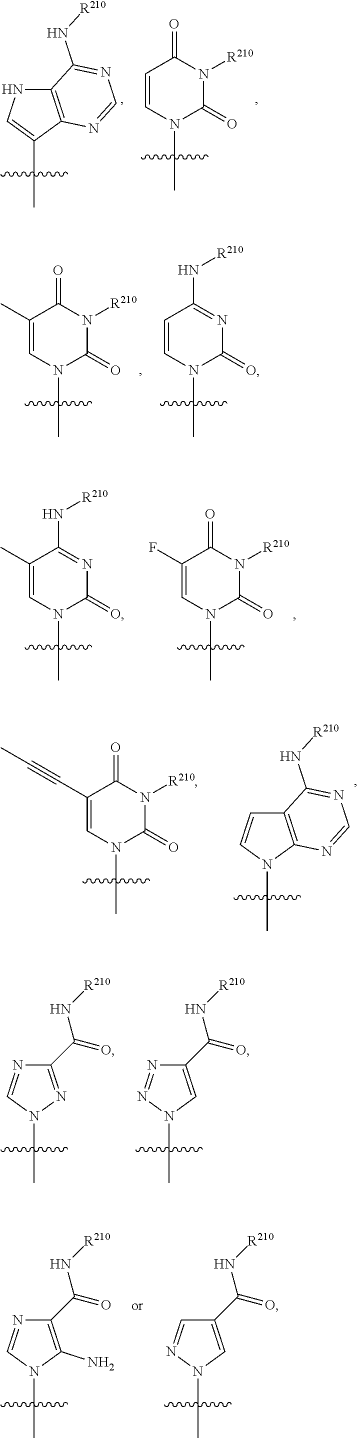

wherein: R^{1b} is substituted with 0, 1, 2 or 3 substituents independently selected from F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂,
and
each R²¹⁰ is independently selected from H and L₁R¹¹⁵;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃;
R^{3a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
R^{4a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
R^{5a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
R^{7a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
each R¹⁰ is independently selected from the group consisting of H, C₁-C₁₂alkyl, —(CH₂CH₂O)ₙCH₂CH₂C(═O)OC₁-C₆alkyl, and

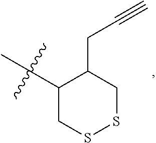 [SMILES: *C1CSSCC1CC#C]

wherein the C₁-C₁₂alkyl of R¹⁰ is substituted by 0, 1, 2 or 3 substituents independently selected from —OH, C₁-C₁₂alkoxy, —S—C(═O)C₁-C₆alkyl and C(O)OC₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, O₂—C₆alkynylene, —O—C₁-C₆alkylene, —O—C₂-C₆alkenylene, —O—C₂-C₆alkynylene, such that when R⁵ and R⁷ are connected, the 0 is bound at the R⁵ position, and
optionally R^{5a} and R^{7a}, are connected to form C₁-C₆alkylene, C₂-C₆alkenylene, O₂—C₆alkynylene, —O—C₁-C₆alkylene, —O—C₂-C₆alkenylene, —O—C₂-C₆alkynylene, such that when R^{5a} and R^{7a} are connected, the 0 is bound at the R^{5a} position;
L₁ is —C(═O)O(CH₂)ₘNR¹¹C(═O)(CH₂)ₘ—**; C(═O)O(CH₂)ₘNR¹¹C(═O)(CH₂)ₘO(CH₂)ₘ—**; —C(═O)O(CH₂)ₘNR¹¹C(═O)X₁X₂C(═O)(CH₂)m-**; —C(═O)OC(R¹²)₂(CH₂)ₘNR¹¹C(═O)X₁X₂C(═O)(CH₂)ₘ—**; —C(═O)O(CH₂)ₘNR¹¹C(═O)X₁X₂C(═O)(CH₂)ₘO(CH₂)ₘ—**; —C(═O)O(CH₂)ₘNR¹¹C(═O)X₁X₂C(═O)(CH₂)ₘO(CH₂)ₘC(═O)**; —C(═O)O(CH₂)ₘNR¹¹C(═O)X₄C(═O)NR¹¹(CH₂)ₘNR¹¹C(═O)(CH₂)ₘO(CH₂)ₘ—**; —C(═O)O(CH₂)ₘNR¹¹C(═O)X₅C(═O)(CH₂)ₘNR¹¹C(═O)(CH₂)ₘ—**; —C(═O)X₄C(═O)NR¹¹ (CH₂)ₘNR¹¹C(═O)(CH₂)ₘO(CH₂)ₘ—**; —C(═O)(CH₂)ₘNR¹¹C(═O)X₁X₂C(═O)(CH₂)ₘ—**; C(═O)(CH₂)ₘNR¹¹C(═O)X₂C(═O)(CH₂)ₘ—**; —C(═O)O(CH₂)ₘX₆C(═O)X₁X₂C(═O)(CH₂)ₘ—**, —C(═O)(CH₂)ₘNR¹¹C(═O)((CH₂)ₘO)ₙ(CH₂)ₘ—**, —C(═O)O(CH₂)ₘX₆C(═O)(CH₂)ₘ—**, —C(═O)O(CH₂)ₘX₆C(═O)(CH₂)ₘO(CH₂)ₘ—**, —C(═O)O(CH₂)ₘX₆C(═O)X₁X₂C(═O)(CH₂)ₘ—**, —C(═O)O(CH₂)ₘX₆C(═O)X₁X₂C(═O)(CH₂)ₘO(CH₂)ₘ—**, —C(═O)O(CH₂)ₘX₆C(═O)X₁X₂C(═O)(CH₂)ₘO(CH₂)ₘC(═O)—**; —C(═O)O(CH₂)ₘX₆C(═O)X₄C(═O)NR¹¹ (CH₂)ₘNR¹¹C(═O)(CH₂)ₘO(CH₂)ₘ—**, —C(═O)X₄C(═O)X₆(CH₂)ₘNR¹¹C(═O) (CH₂)ₘO(CH₂)ₘ—**, —C(═O)(CH₂)ₘX₆C(═O)X₁X₂C(═O)(CH₂)ₘ—**, —C(═O)O((CH₂)ₘO)ₙ(CH₂)ₘNR¹¹C(═O)X₅C(═O)(CH₂)ₘ—**; —C(═O)O((CH₂)ₘO)ₙ(CH₂)ₘNR¹¹C(═O)X_{C}(═O)(CH₂)ₘNR¹¹C(═O)(CH₂)ₘ—**; —C(═O)O((CH₂)ₘO)ₙ(CH₂)ₘNR¹¹C(═O)X₅C(═O)(CH₂)ₘX₃(CH₂)ₘ—**; —C(═O)O((CH₂)ₘO)ₙ(CH₂)ₘNR¹¹C(═O)X_{C}(═O)((CH₂)ₘO)ₙ(CH₂)ₘ—**; —C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘ—**; C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)O((CH₂)ₘO)ₙ(CH₂)ₘNR¹¹C(═O))X₅C(═O)((CH₂)ₘO)ₙ(CH₂)ₘX₃(CH₂)ₘ—**; —C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘ—**; C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O)O((CH₂)ₘO)ₙ(CH₂)ₘNR¹¹C(═O)X(CH₂)ₘX₃(CH₂)ₘ—**; —C(═O)O((CH₂)ₘO)ₙ(CH₂)ₘNR¹¹C(═O)X₅((CH₂)ₘO)ₙ(CH₂)ₘ—**; —C(═O)O((CH₂)ₘO)ₙ(CH₂)ₘNR¹¹C(═O)X₅((CH₂)ₘO)ₙ(CH₂)ₘNR¹¹C(═O)(CH₂)ₘ—**; —C(═O)O((CH₂)ₘO)ₙ(CH₂)ₘNR¹¹C(═O)X₅((CH₂)ₘO)ₙ(CH₂)ₘNR¹¹C(═O)(CH₂)ₘX₃(CH₂)ₘ—**; —C(═O)O((CH₂)ₘO)ₙ(CH₂)ₘNR¹¹C(═O)X₅((CH₂)ₘO)ₙ(CH₂)ₘX₃(CH₂)ₘ—**; —C(═O)O((CH₂)ₘO)ₙ(CH₂)ₘNR¹¹C(═O)X₅(CH₂)ₘNR¹¹((CH₂)ₘO)ₙ(CH₂)ₘ—**; —C(═O)O((CH₂)ₘO)ₙ(CH₂)ₘNR¹¹C(O)X₅C(═O)(CH₂)ₘNR¹¹ ((CH₂)ₘO)ₙ(CH₂)ₘX₃(CH₂)ₘ—**; —C(═O)O((CH₂)ₘO)ₙ(CH₂)ₘNR¹¹C(═O)X(CH₂)ₘ—**; —C(═O)O((CH₂)ₘO)ₙ(CH₂)ₘNR¹¹C(═O)X_{C}(═O)((CH₂)ₘO)ₙ(CH₂)ₘ—**; —C(═O)O((CH₂)ₘO)ₙ(CH₂)ₘNR¹¹C(═O)X(CH₂)ₘX₃(CH₂)ₘ—**; —C(═O)O(CH₂)ₘ—**; C(═O)O((CH₂)ₘO)ₙ(CH₂)ₘ—**; —C(═O)O(CH₂)ₘNR¹¹ (CH₂)ₘ—**; —C(═O)O(CH₂)ₘNR¹¹(CH₂)ₘC(═O)X₂X_{C}(═O)—**; C(═O)O(CH₂)ₘX₃(CH₂)ₘ—**; —C(═O)O(H₂)ₘX₆C(═O)X₁X₂C(═O)((CH₂)ₘO)ₙ(CH₂)ₘ—**; —C(═O)O((CH₂)ₘO)ₙ(CH₂)ₘX₃(CH₂)ₘ—**; C(═O)O((CH₂)ₘO)ₙ(CH₂)ₘNR¹¹C(═O)(CH₂)ₘ—**; —C(═O)O(CH₂)ₘNR¹¹C(═O(CH₂)ₘX₃(CH₂)ₘ—**; —C(═O)O((CH₂)ₘO)ₙ(CH₂)ₘNR¹¹C(═O)(CH₂)ₘX(CH₂)ₘ—**; —C(═O)O((CH₂)ₘO)ₙX₃(CH₂)ₘ—**; C(═O)O((CH₂)ₘO)ₙ(CH₂)ₘX₃(CH₂)ₘ—**; —C(═O)O((CH₂)ₘO)ₙ(CH₂)ₘC(═O)NR¹¹(CH₂)ₘ—**; C(═O)O(CH₂)ₘC(R¹²)₂—**; —C(═O)OCH₂)ₘC(R¹²)₂SS(CH₂)ₘNR¹¹C(═O)(CH₂)ₘ—**; —C(═O)O(CH₂)ₘC(═O)NR¹¹ (CH₂)ₘ—**; —C(═O)(CH₂)ₘ—**; C(═O)((CH₂)ₘO)ₙ(CH₂)ₘ—**; —C(═O)(CH₂)ₘNR¹¹(CH₂)ₘ—**; —C(═O)(CH₂)ₘNR¹¹ (CH₂)ₘC(═O)X₂X₁C(═O)—**; —C(═O)(CH₂)ₘX₃(CH₂)ₘ**; —C(═O)((CH₂)ₘO)ₙ(CH₂)ₘX₃(CH₂)ₘ—**; C(═O)(CH)ₘNR¹¹C(═O)(CH)ₘ**; —C(═O)((CH₂)ₘO)ₙ(CH₂)ₘNR¹¹C(═O)(CH₂)ₘ—**; C(═O)(CH₂)ₘNR¹¹C(O(CH₂)ₘX(CH₂)ₘ—**; (CH₂)ₘNR¹¹C(═O)X₁X₂C(═O)(CH₂)ₘ—**. —(CH₂)ₘ(CHOH)(CH₂)ₘNR¹¹C(═O)X₁X₂C(═O)(CH₂)ₘ**; —C(═O)((CH₂)ₘO)ₙ(CH₂)ₘNR¹¹C(═O)(CH₂)ₘX₃(CH₂)ₘ—**; —C(═O)((CH₂)ₘO)ₙX₃(CH₂)ₘ—**; C(═O)((CH₂)ₘO)ₙ(CH₂)ₘX₃(CH₂)ₘ—**; —C(═O)((CH₂)ₘO)ₙ(CH₂)ₘC(═O)NR¹¹ (CH₂)ₘ—**; C(═O)(CH₂)ₘC(R¹²)₂—**; —C(═O)((CH₂)ₘO)ₙ(CH₂)ₘNR¹¹C(═O)X₅C(═O)(CH₂)ₘ—**; —C(═O)((CH₂)ₘO)ₙ(CH₂)ₘNR¹¹C(═O)X₅C(═O)(CH₂)ₘNR¹¹C(═O)(CH₂)ₘ**; —C(═O) ((CH₂)ₘO)ₙ(CH₂)ₘNR¹¹C(═O)X₅C(═O)(CH₂)ₘX₃(CH₂)ₘ—**; —C(═O)((CH₂)ₘO)ₙ(CH₂)ₘNR¹¹C(═O)X₅C(═O)((CH₂)ₘO)ₙ(CH₂)ₘ—**; —C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘ—**; —C(═O)((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O) ((CH₂)ₘO)ₙ (CH₂)ₘNR¹¹C(═O))X₅C(═O) ((CH₂)ₘO)ₙ(CH₂)ₘX₃(CH₂)ₘ—**; —C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘ—** —C(═O)((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘX₃(CH₂)ₘ—**; —C(═O)((CH₂)ₘO)ₙ(CH₂)ₘNR¹¹C(═O)X₅((CH₂)ₘO)ₙ(CH₂)ₘ—**; —C(═O)((CH₂)ₘO)ₙ(CH₂)ₘNR¹¹C(═O)X₅((CH₂)ₘO)ₙ(CH₂)ₘNR¹¹C(═O)(CH₂)ₘ—**; —C(═O)((CH₂)ₘO)ₙ(CH₂)ₘNR¹¹C(═O)X₅((CH₂)ₘO)ₙ(CH₂)ₘNR¹¹C(═O)(CH₂)ₘX₃(CH₂)ₘ—**; —C(═O)((CH₂)ₘO)ₙ(CH₂)ₘNR¹¹C(═O)X₅((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘNR¹¹ ((CH₂)ₘO)ₙ(CH₂)ₘ—**; —C(═O) ((CH₂)ₘO)ₙ(CH₂)ₘNR¹¹C(═O)X₅C(═O)(CH₂)ₘNR¹¹ ((CH₂)ₘO)ₙ(CH₂)ₘX₃(CH₂)ₘ—**; —C(═O)((CH₂)ₘO)ₙ(CH₂)ₘNR¹¹C(═O)X₅(CH₂)ₘ—**; —C(═O)((CH₂)ₘO)ₙ(CH₂)ₘNR¹¹C(═O)X₅C(═O)((CH₂)ₘO)ₙ(CH₂)ₘ—**; —C(═O)((CH₂)ₘO)ₙ(CH₂)ₘNR¹¹C(═O)X₅(CH₂)ₘX₃(CH₂)ₘ—**; —C(═O)(CH₂)ₘC(R¹²)₂SS(CH₂)ₘNR¹¹C(═O)(CH₂)ₘ—**; —C(═O)(CH₂)ₘC(═O)NR¹¹(CH₂)ₘ—**; C(═O)X₁X₂C(═O)(CH₂)ₘ—**; —C(═O)X₁X₂C(═O)(CH₂)ₘNR¹¹C(═O)(CH₂)ₘ—**; C(═O)X₁X₂C(═O)(CH₂)ₘX₃(CH₂)ₘ*. —C(═O)X₁X₂C(═O)((CH₂)ₘO)ₙ(CH₂)ₘ—**; —C(═O)X₁X₂C(═O)((CH₂)ₘO)ₙ(CH₂)ₘNR¹¹C(═O)(CH₂)ₘ—**; —C(═O)X₁X₂C(═O)((CH₂)ₘO)ₙ(CH₂)ₘNR¹¹C(═O)(CH₂)ₘX₃(CH₂)ₘ—**; —C(═O)X₁X₂C(═O)((CH₂)ₘO)ₙ(CH₂)ₘX₃(CH₂)ₘ—**; —C(═O)X₁X₂C(═O)(CH₂)ₘNR¹¹C(═O)((CH₂)ₘO)ₙ(CH₂)ₘ—**; —C(═O)X₁X₂C(═O)(CH₂)ₘNR¹¹C(═O) ((CH₂)ₘO)ₙ(CH₂)ₘX₃(CH₂)ₘ—**; —C(═O)X₁X₂(CH₂)ₘX₃(CH₂)ₘ—**; C(═O)X₁X₂((CH₂)ₘO)ₙ(CH₂)ₘ—**; —C(═O)X₁X₂((CH₂)ₘO)ₙ(CH₂)ₘNR¹¹C(═O)(CH₂)ₘ—**; —C(═O)X₁X₂((CH₂)ₘO)ₙ(CH₂)ₘNR¹¹C(═O)(CH₂)ₘX₃(CH₂)ₘ—**; —C(═O)X₁X₂((CH₂)ₘO)ₙ(CH₂)ₘX₃(CH₂)ₘ—**; —C(═O)X₁X₂(CH₂)ₘNR¹¹ ((CH₂)ₘO)ₙ(CH₂)ₘ—**; —C(═O)X₁X₂C(═O)(CH₂)ₘNR¹¹ ((CH₂)ₘO)ₙ(CH₂)ₘX₃(CH₂)ₘ—**; —C(═O)NR¹¹(CH₂)ₘ—**; C(═O)NR¹¹(CH₂)ₘX₃(CH₂)ₘ—**; —C(═O)NR¹¹ (CH₂)ₘNR¹¹C(═O)X₁X₂C(═O)(CH₂)ₘ—**; —C(═O)NR¹¹(CH₂)ₘNR¹¹C(═O)O(CH₂)ₘ—**; —C(═O)NR¹¹ (CH₂)ₘNR¹¹C(═O)X₁X₂—**; C(═O)NR¹¹ (CH₂)ₘNR¹¹C(═O)X₅—; —C(═O)NR¹¹ (CH₂)ₘNR¹¹C(═O)(CH₂)ₘX₅(CH₂)ₘ—**; —C(═O)X₁C(═O)NR¹¹ (CH₂)ₘX₃(CH₂)ₘ—**; —C(═O)NR¹¹ (CH₂)ₘNR¹¹C(═O)(CH₂)ₘ—**; —C(═O)NR¹¹(CH₂)ₘNR¹¹C(═O)(CH₂)ₘO(CH₂)ₘ—**; —C(═O)NR¹¹ (CH₂)ₘNR¹¹C(═O)X₁X₂C(═O)(CH₂)ₘO(CH₂)ₘ—**; —C(═O)NR¹¹ (CH₂)ₘNR¹¹C(═O)X₁X₂C(═O)(CH₂)ₘO(CH₂)ₘC(═O)—**; —C(═O)NR¹¹(CH₂)ₘNR¹¹C(═O)X₄C(═O)NR¹¹(CH₂)ₘNR¹¹C(═O)(CH₂)ₘO(CH₂)ₘ—**; —C(═O)NR¹¹(CH₂)ₘNR¹¹C(═O)X₁X₂C(═O) (CH₂)ₘ—**; —C(═O)NR¹¹(CH₂)ₘNR¹¹C(═O)X₅C(═O)(CH₂)ₘ—**; —C(═O)NR¹¹(CH₂)ₘNR¹¹C(═O)X₅C(═O)(CH₂)ₘNR¹¹C(═O)(CH₂)ₘ—**; —C(═O)NR(CH₂)ₘNR¹¹C(═O)X₅C(═O)(CH₂)ₘX₃(CH₂)ₘ—**; —C(═O)NR¹¹(CH₂)ₘNR¹¹C(═O)X₅C(═O)((CH₂)ₘO)ₙ(CH₂)ₘ—**; —C(═O)NR¹¹(CH₂)ₘNR¹¹C(═O)X₅C(═O)((CH₂)ₘO)ₙ(CH₂)ₘNR¹¹C(═O)(CH₂)ₘ—**; —C(═O)NR¹¹(CH₂)ₘNR¹¹C(═O)X₅C(═O)((CH₂)ₘO)ₙ(CH₂)ₘNR¹¹C(═O)(CH₂)ₘX₃(CH₂)ₘ—**; —C(═O)NR¹¹(CH₂)ₘNR¹¹C(═O)X₅C(═O) ((CH₂)ₘO)ₙ(CH₂)ₘX₃(CH₂)ₘ—**; —C(═O)NR¹¹(CH₂)ₘNR¹¹C(═O)X₅C(═O)(CH₂)ₘNR¹¹C(═O)((CH₂)ₘO)ₙ(CH₂)ₘ—**; —C(═O)NR¹¹(CH₂)ₘNR¹¹C(═O)X₅C(═O)(CH₂)ₘNR¹¹C(═O)((CH₂)ₘO)ₙ(CH₂)ₘX₃(CH₂)ₘ—**; —C(═O) NR¹¹(CH₂)ₘNR¹¹C(═O)X₅(CH₂)ₘX₃(CH₂)ₘ—**; —C(═O)NR¹¹(CH₂)ₘNR¹¹C(═O)X₅((CH₂)ₘO)ₙ(CH₂)ₘ—**; —C(═O)NR¹¹(CH₂)ₘNR¹¹C(═O)X₅((CH₂)ₘO)ₙ(CH₂)ₘNR¹¹C(═O)(CH₂)ₘ—**; —C(═O)NR¹¹(CH₂)ₘNR¹¹C(═O)X₅((CH₂)ₘO)ₙ(CH₂)ₘNR¹¹C(═O)(CH₂)ₘX(CH₂)ₘ—**; —C(═O) NR¹¹(CH₂)ₘNR¹¹C(═O)X₅((CH₂)ₘO)ₙ(CH₂)ₘX₃(CH₂)ₘ—**; —C(═O)NR¹¹(CH₂)ₘNR¹¹C(═O)X₅(CH₂)ₘNR¹¹((CH₂)ₘO)ₙ(CH₂)ₘ—**; —C(═O)NR¹¹(CH₂)ₘNR¹¹C(═O)X₅C(═O)(CH₂)ₘNR¹¹((CH₂)ₘO)ₙ(CH₂)ₘX₃(CH₂)ₘ—**; —C(═O) NR¹¹ (CH₂)ₘNR¹¹C(═O)X₅(CH₂)ₘ—**; —C(═O)NR(CH₂)ₘNR¹¹C(═O)X₅C(═O)((CH₂)ₘO)ₙ(CH₂)ₘ—**; —C(═O) NR¹¹(CH₂)ₘNR¹¹C(═O)X₅(CH₂)ₘX₃(CH₂)ₘ—**; —C(═O)X₁C(═O)NR¹¹(CH₂)ₘNR¹¹C(═O)(CH₂)ₘ—**; —C(═O)X₁C(═O)NR¹¹(CH₂)ₘX₃(CH₂)ₘ—**; C(═O)NR¹¹(CH₂)ₘNR¹¹C(═O)(CH₂)ₘ—**. —C(═O)NR¹¹(CH₂)ₘNR¹¹C(═O)(CH₂)ₘX₃(CH₂)ₘ—**; C(═O)NR¹¹(CH₂)ₘNR¹¹C(═O)—**; —C(═O)X₁X₂(CH₂)ₘ—**; C(═O)X₁X₂C(═O)((CH₂)ₘO)ₙ(CH₂)ₘ—**; —C(═O)X₁X₂(CH₂)ₘX₃(CH₂)ₘ—**; C(═O)NR¹¹ (CH₂)ₘX₃(CH₂)ₘ**; —C(═O)NR¹¹((CH₂)ₘO)ₙ(CH₂)ₘX₃(CH₂)ₘ—**; C(═O)X₁X₂C(═O)((CH₂)ₘO)ₙ(CH₂)ₘ—**; —C(═O)X₁X₂C(═O)(CH₂)ₘ—**; —C(═O)X₁C(═O)(CH₂)ₘNR¹¹C(═O)(CH₂)ₘ—**; and —C(═O)X₁C(═O)(CH₂)ₘNR¹¹C(═O)((CH₂)ₘO)ₙ(CH₂)ₘ—**;
   where the ** of L₁ indicates the point of attachment to R¹¹⁵;
R¹¹⁵ is

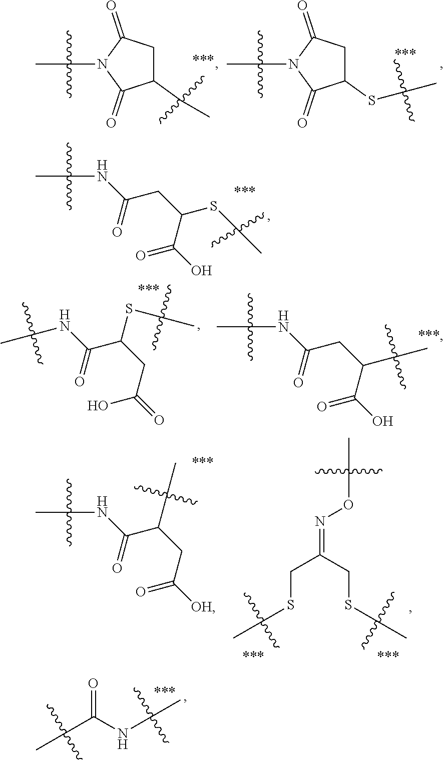

—C(═O)—, —ON═***, —S—, —NHC(═O)CH₂—, —S(═O)₂CH₂CH₂—, —(CH₂)₂S(═O)₂CH₂CH₂—, —NHS(═O)₂CH₂CH₂, —NHC(═O)CH₂CH₂—, —CH₂NHCH₂CH₂—, —NHCH₂CH₂—,

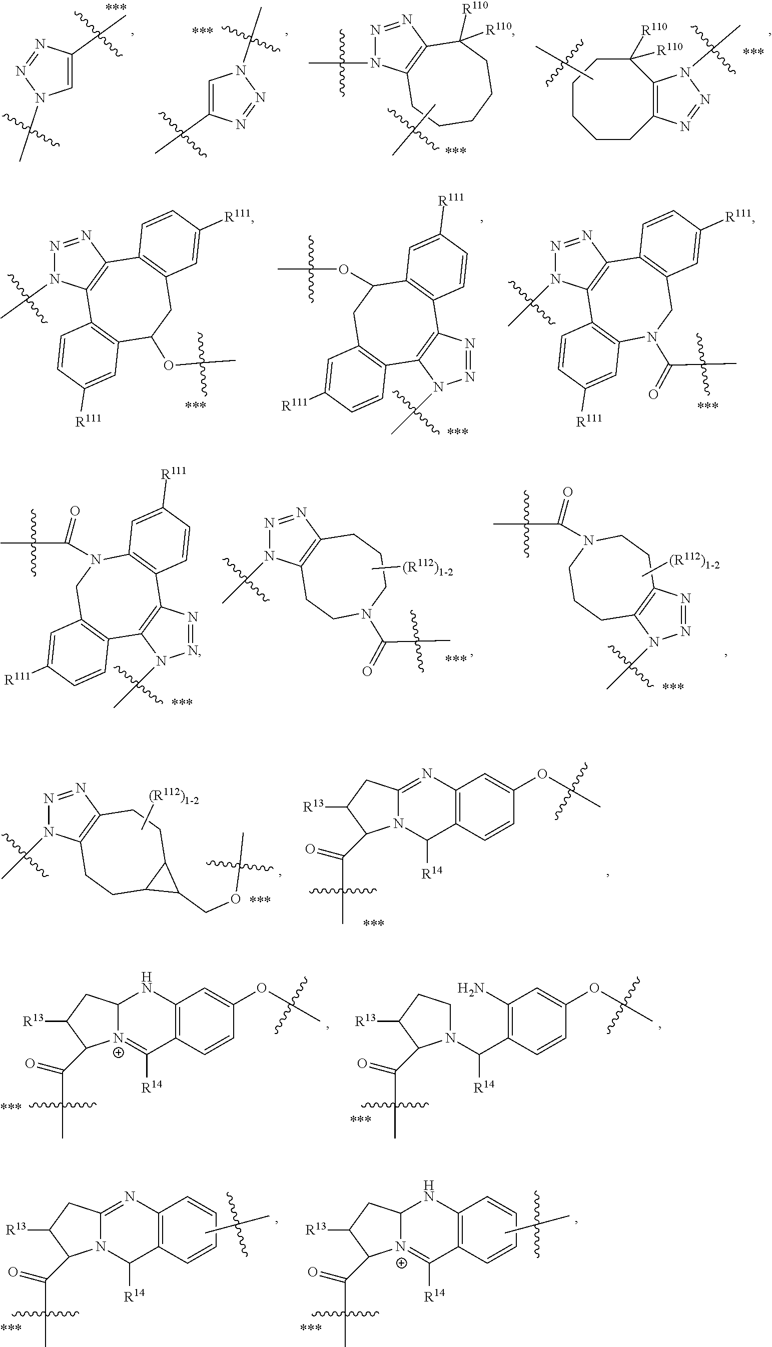

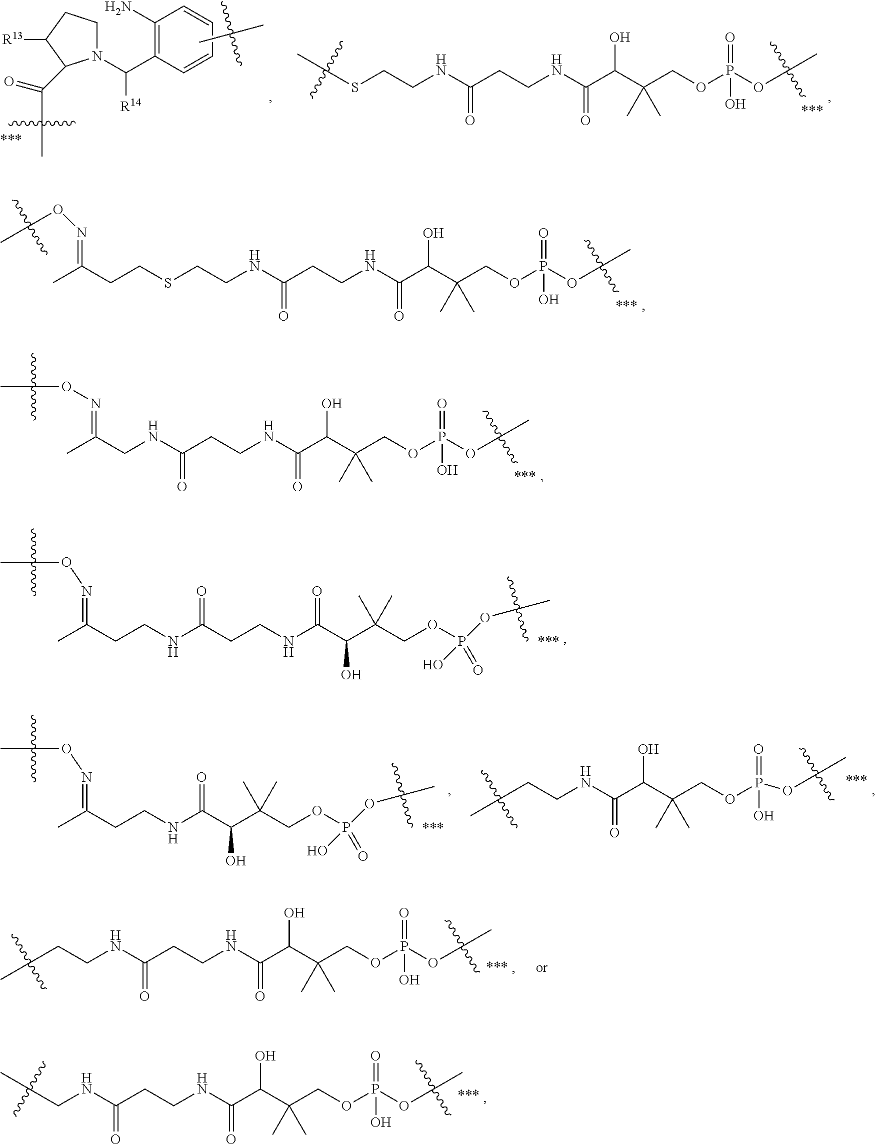

where the *** of R¹¹⁵ indicates the point of attachment to Ab;
X₁ is

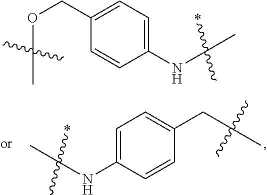

where the * of X₁ indicates the point of attachment to X₂;
X₂ is selected from

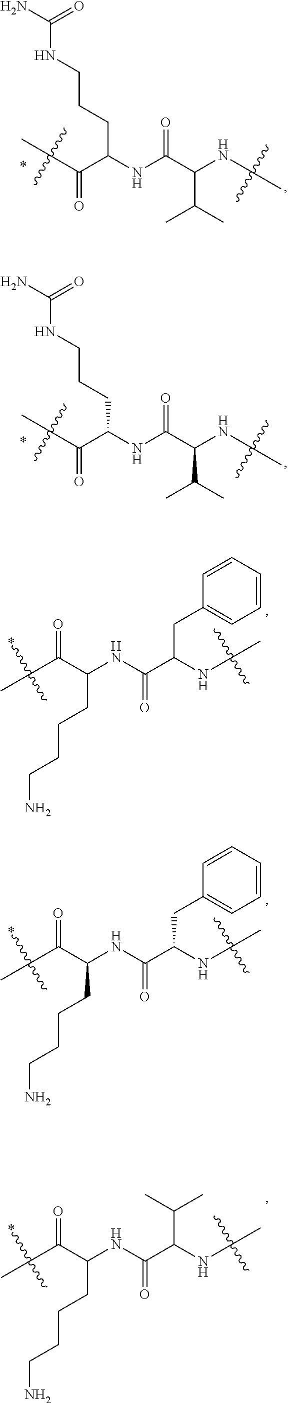 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O ; *NC(C(=O)NC(CCCCN)C(*)=O)C(C)C]

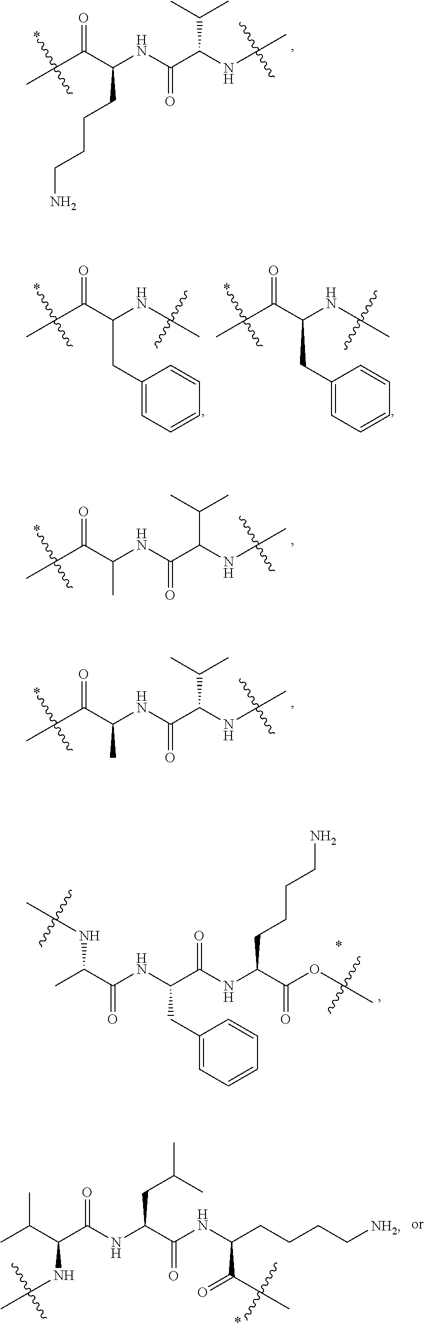

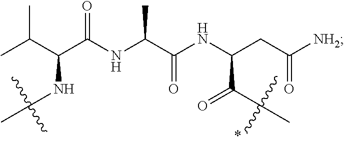 [SMILES: *N[C@H](C(=O)N[C@@H](C)C(=O)N[C@@H](CC(N)=O)C(*)=O)C(C)C]

where the * of X₂ indicates the point of attachment to X₁;
X₃ is

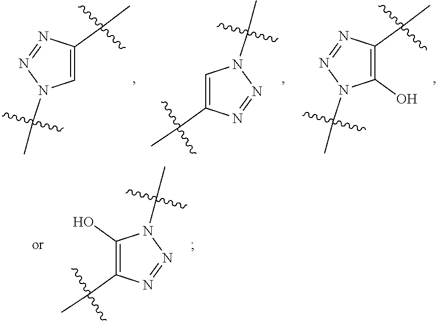 [SMILES: *c1cn(*)nn1 ; *c1cn(*)nn1 ; *c1nnn(*)c1O ; *c1nnn(*)c1O]

X₄ is —O(CH₂)ₙSSC(R¹²)₂(CH₂)ₙ— or —(CH₂)ₙC(R¹²)₂SS(CH₂)ₙO—;
X₅ is

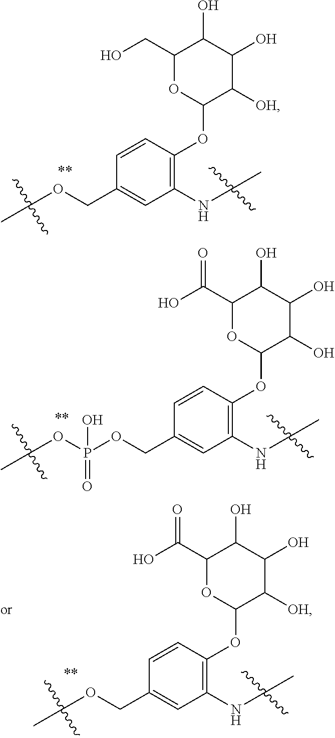 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the ** of X₅ indicates orientation toward R¹¹⁵;
X₆ is

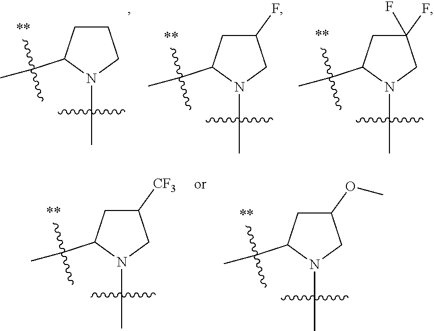

or, where the ** of X₆ indicates orientation toward R¹¹⁵;
each R¹¹ is independently selected from H and C₁-C₆alkyl;
each R¹² is independently selected from H and C₁-C₆alkyl;
R¹³ is H or methyl;
R¹⁴ is H, —CH₃ or phenyl;
each R¹¹⁰ is independently selected from H, C₁-C₆alkyl, F, Cl, and —OH;
each R¹¹¹ is independently selected from H, C₁-C₆alkyl, F, Cl, —NH₂, —OCH₃, —OCH₂CH₃, —N(CH₃)₂, —CN, —NO₂ and —OH;
each R¹¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, 4, 5, 6, 7, 8, 9 and 10; each n is independently selected from 1, 2, 3, 4, 5, 6, 7, 8, 9, 10, 11, 12, 13, 14, 15, 16, 17 and 18.
Ab is an anti-DC-SIGN antibody or fragment thereof, and
each y is independently selected from 1, 2, 3, 4, 5, 6, 7, 8, 9 or 10,
and provided at least one of R²⁰⁰ or R²¹⁰ is -L₁R¹¹⁵ or is substituted with —NHL₁R¹¹⁵, or at least one of R³, R⁴, R⁵, R⁷, R^{3a}, R^{4a}, R^{5a} or R^{7a} is —OL₁R¹¹⁵

### Embodiment 206

A DC-SIGN immunoconjugate selected from:

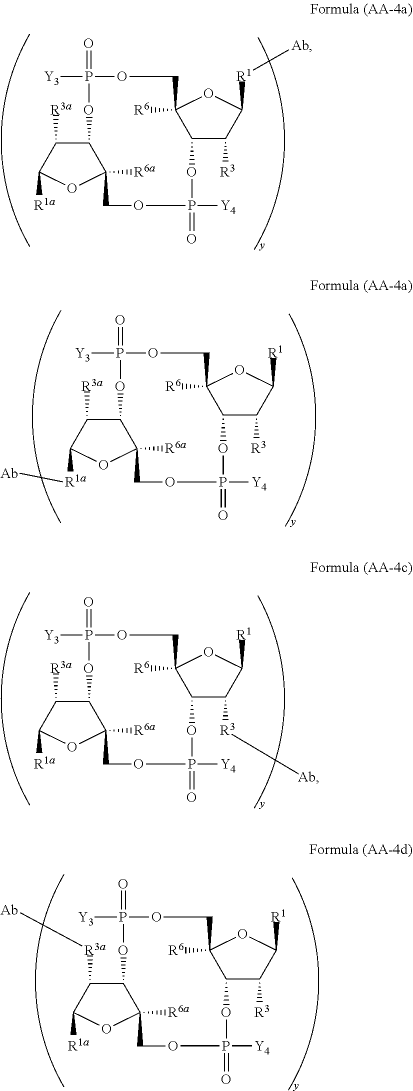

wherein: Ab, y, R¹, R^{1a}, R³, R^{3a}, R⁶, R^{6a}, Y₃ and Y₄ are as defined in Embodiment 205.

### Embodiment 207

A DC-SIGN immunoconjugate selected from:

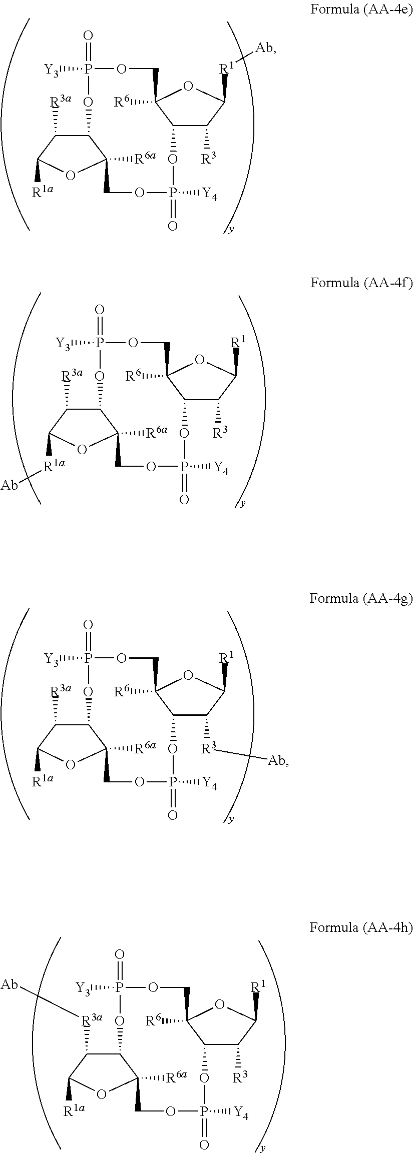

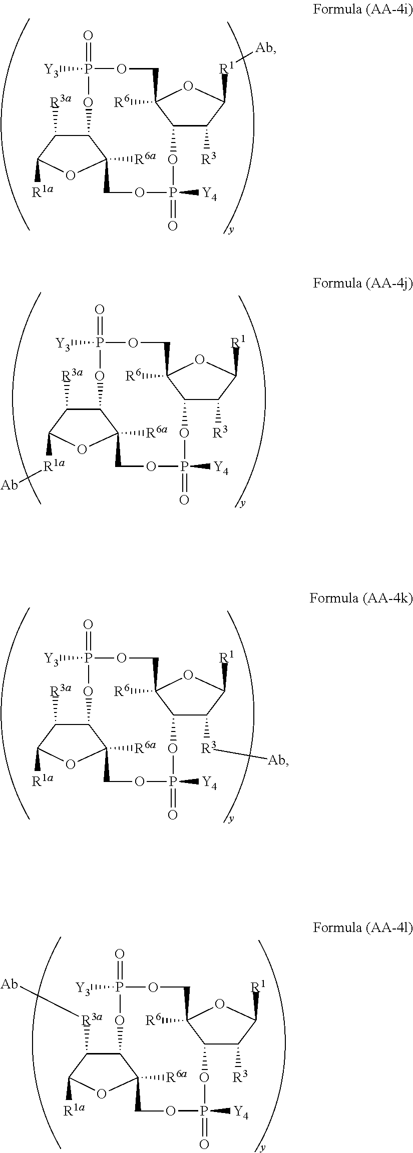

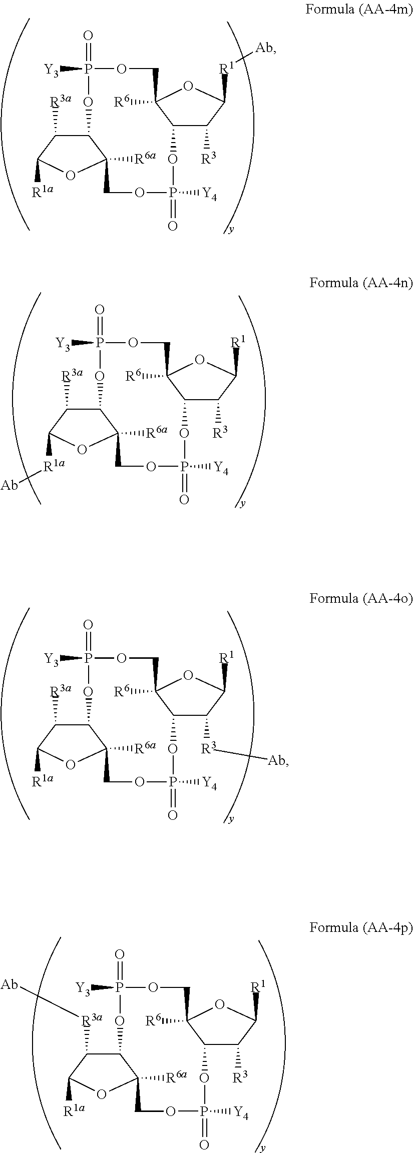

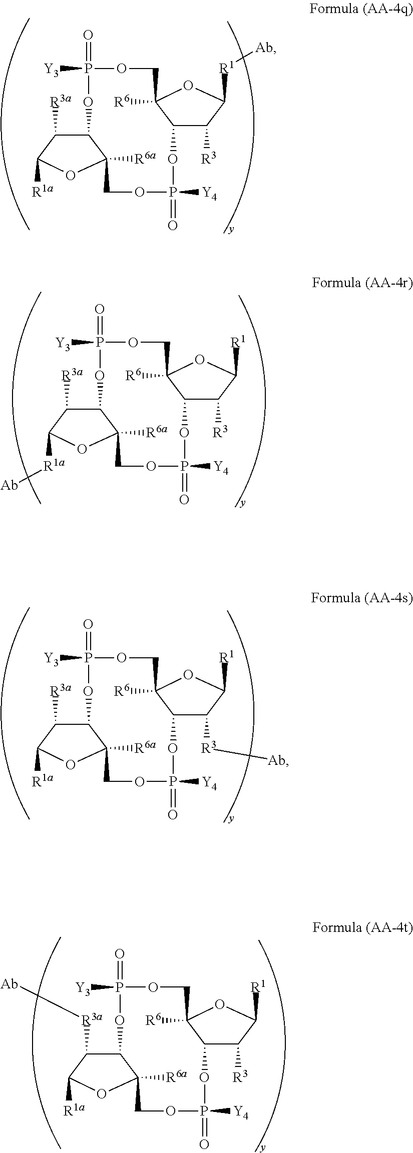

wherein: Ab, y, R¹, R^{1a}, R³, R^{3a}, R⁶ and R^{6a} are as defined in Embodiment 205;
Y₃ is OR⁹, N(R¹⁰)₂, SH or S⁻, and
Y₄ is OR⁹, N(R¹⁰)₂, SH or S⁻.

### Embodiment 208

A DC-SIGN immunoconjugate selected from:

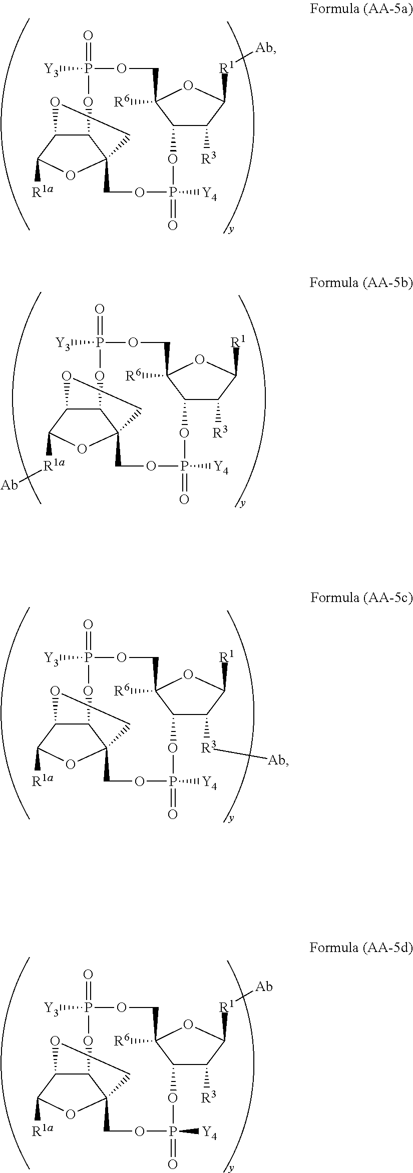

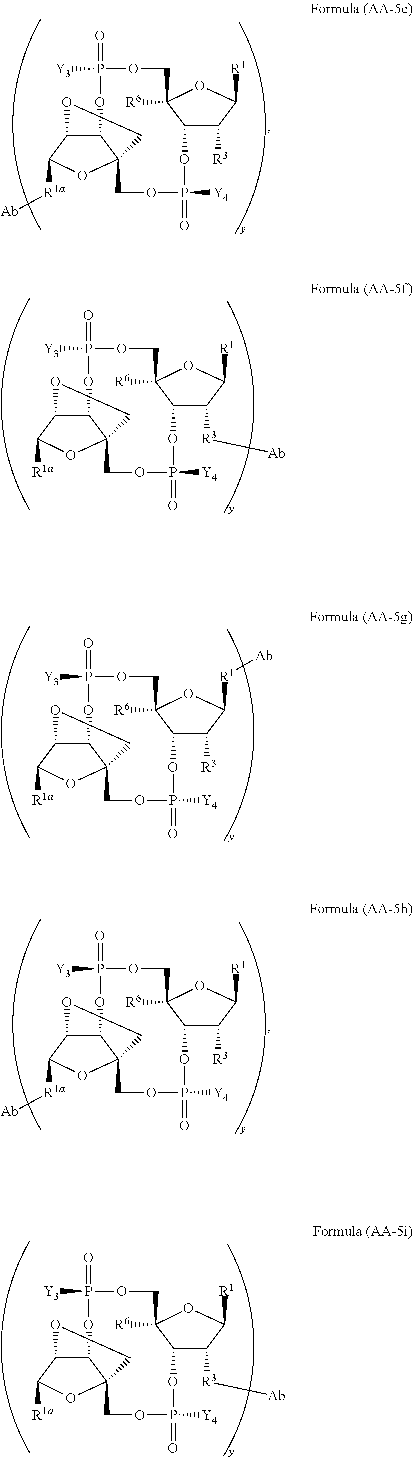

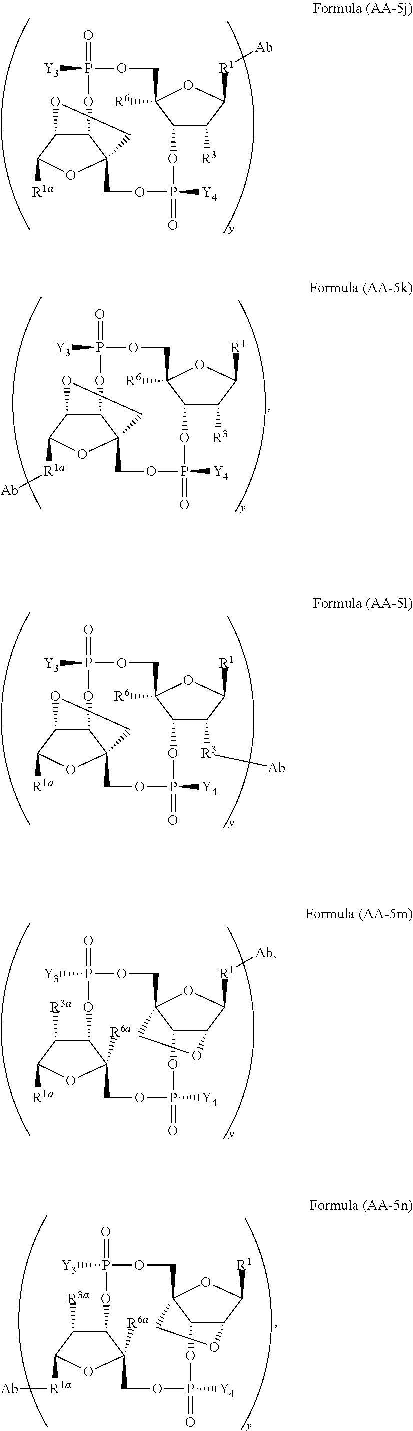

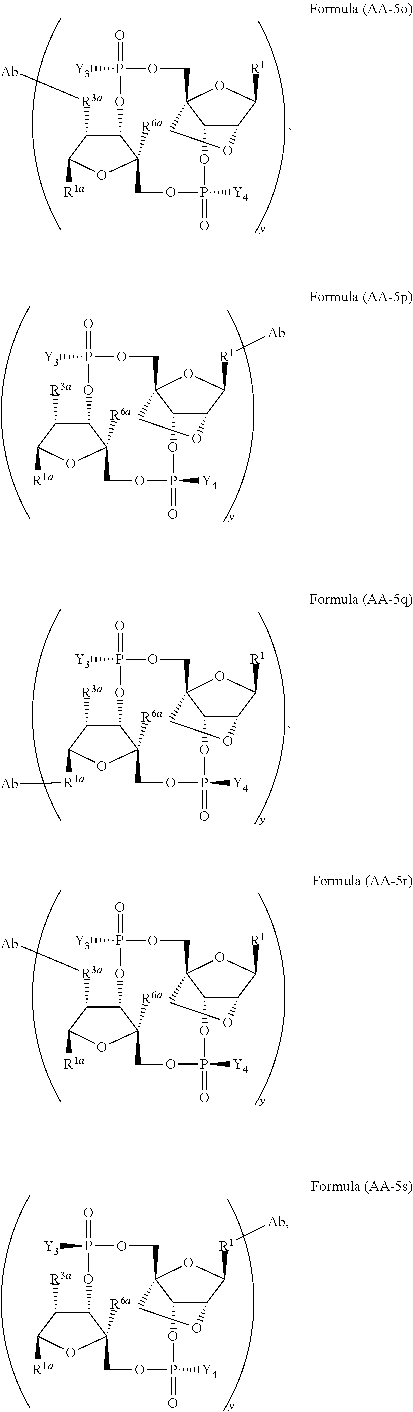

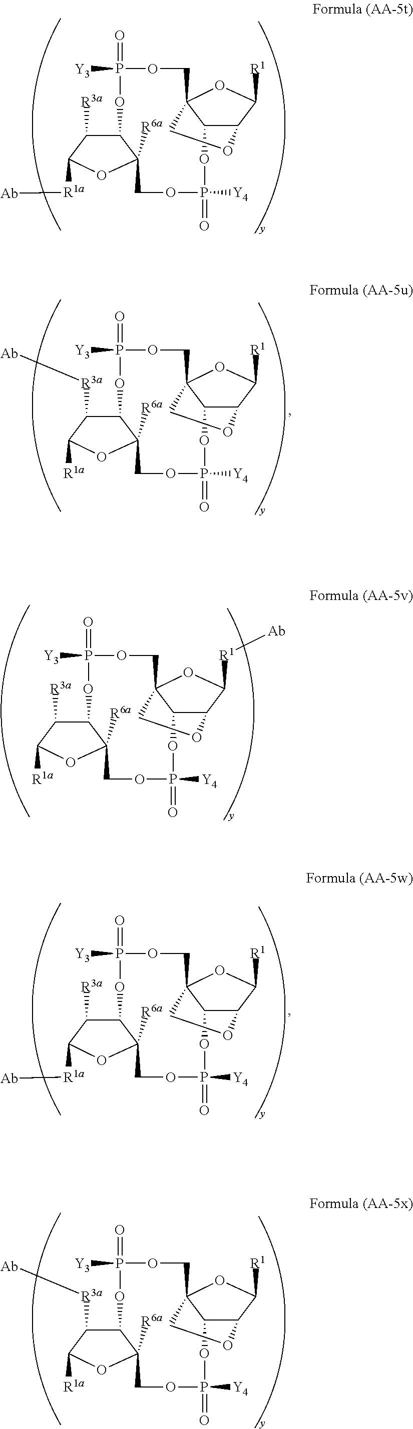

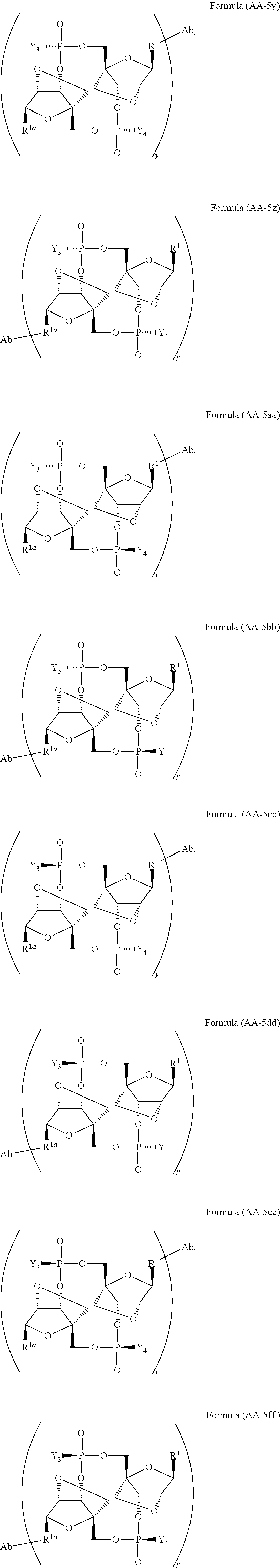

wherein: Ab, y, R¹, R^{1a}, R³, R^{3a}, R⁶ and R^{6a} are as defined in Embodiment 205;
Y₃ is OR⁹, N(R¹⁰)₂, SH or S⁻, and
Y₄ is OR⁹, N(R¹⁰)₂, SH or S⁻.

### Embodiment 209

An immunoconjugate selected from:

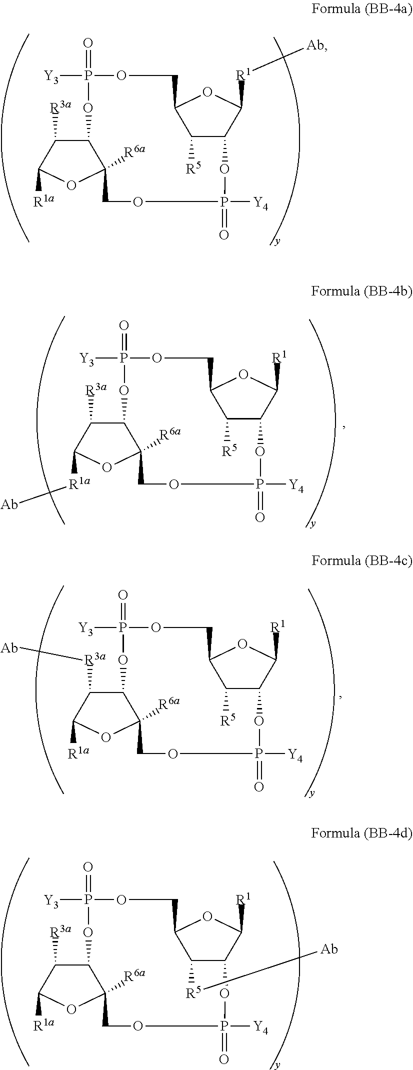

wherein: Ab, y, R¹, R^{1a}, R³, R^{3a}, R⁵, R^{6a}, Y₃ and Y₄ are as defined in Embodiment 205.

### Embodiment 210

A DC-SIGN immunoconjugate selected from:

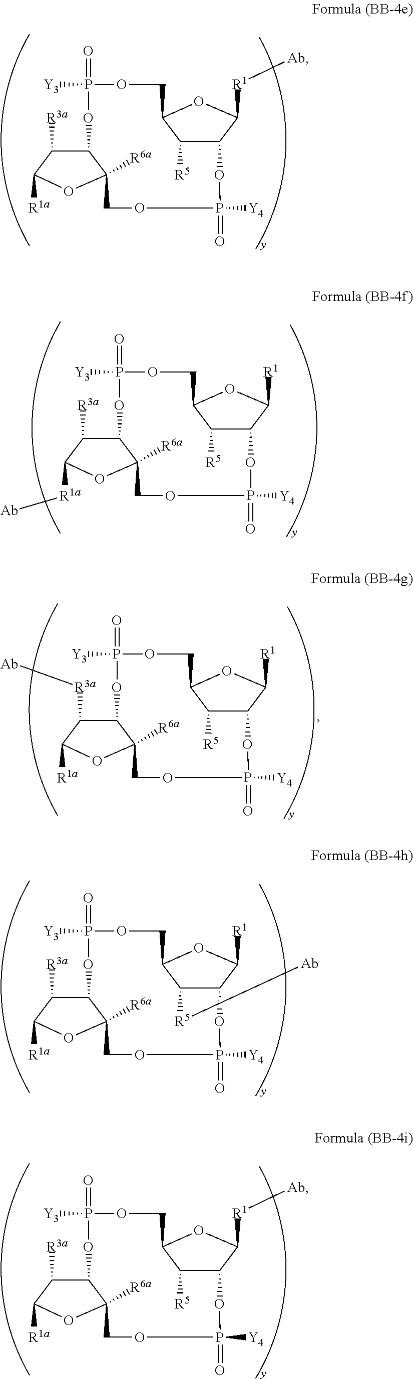

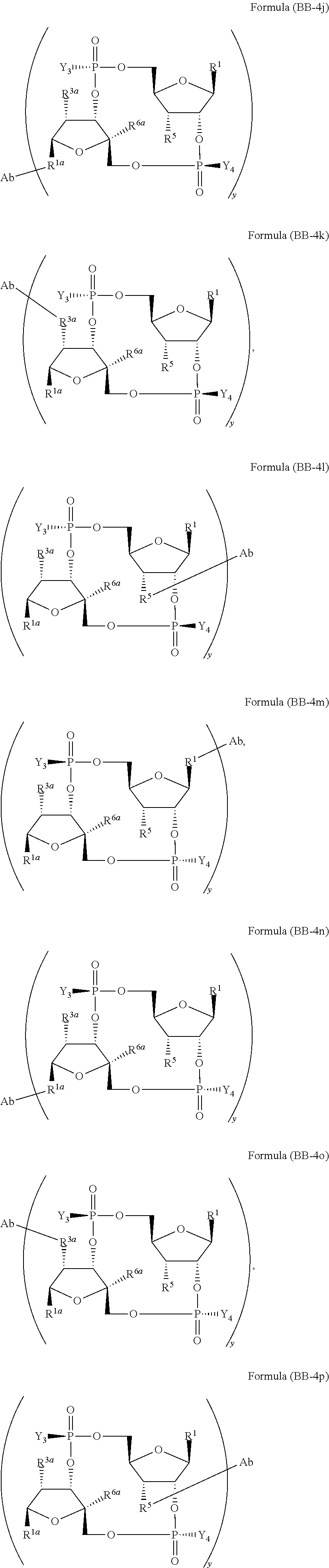

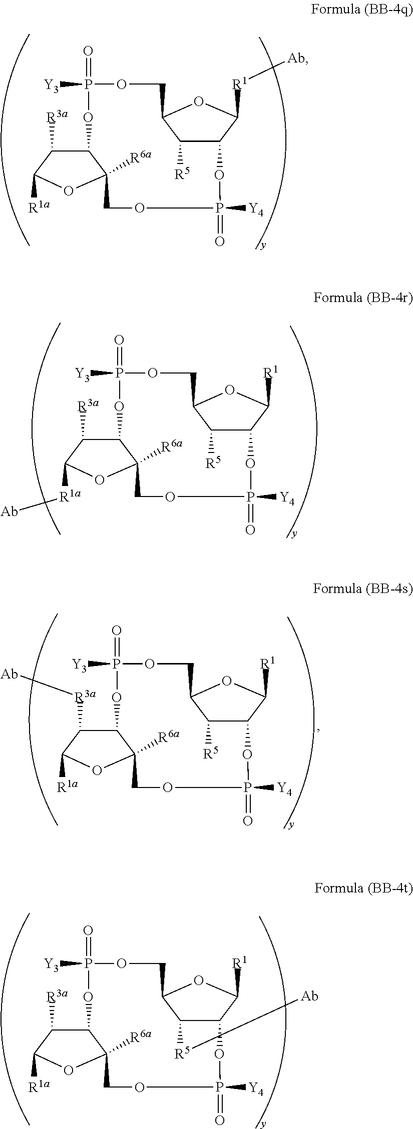

wherein: Ab, y, R¹, R¹, R^{3a}, R⁵ and R^{6a} are as defined in Embodiment 205;
Y₃ is OR⁹, N(R¹⁰)₂, SH or S⁻, and
Y₄ is OR⁹, N(R¹⁰)₂, SH or S⁻.

### Embodiment 211

A DC-SIGN immunoconjugate selected from:

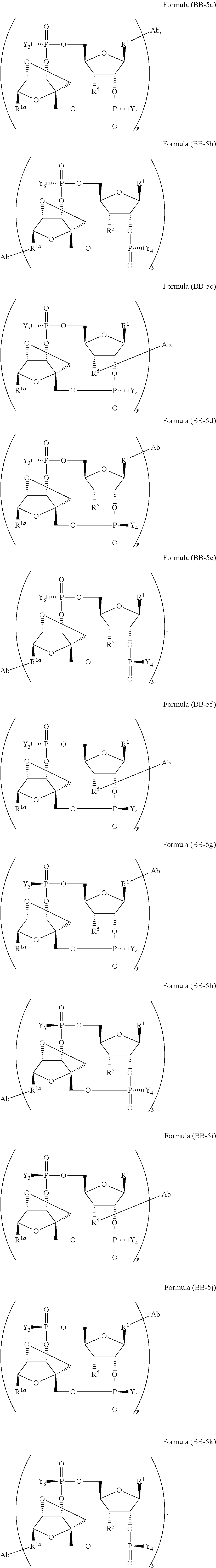

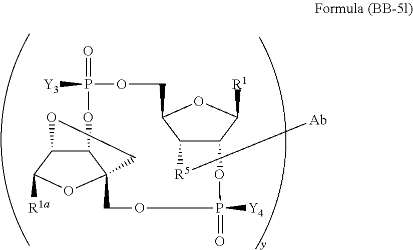

wherein: Ab, y, R¹, R^{1a} and R⁵ are as defined in Embodiment 205;
Y₃ is OR⁹, N(R¹⁰)₂, SH or S⁻, and
Y₄ is OR⁹, N(R¹⁰)₂, SH or S⁻.

### Embodiment 212

A DC-SIGN immunoconjugate selected from:

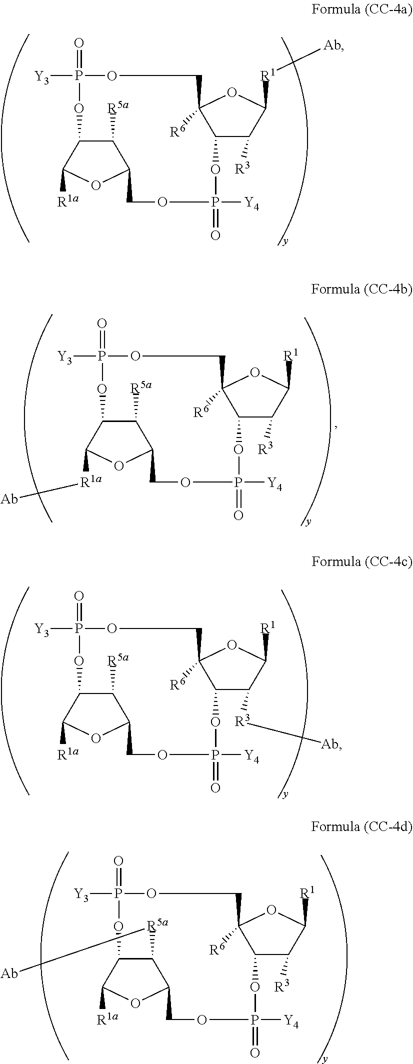

wherein: Ab, y, R¹, R^{1a}, R³, R^{5a}, R⁶, R^{6a}, Y₃ and Y₄ are as defined in Embodiment 205.

### Embodiment 213

A DC-SIGN immunoconjugate selected from:

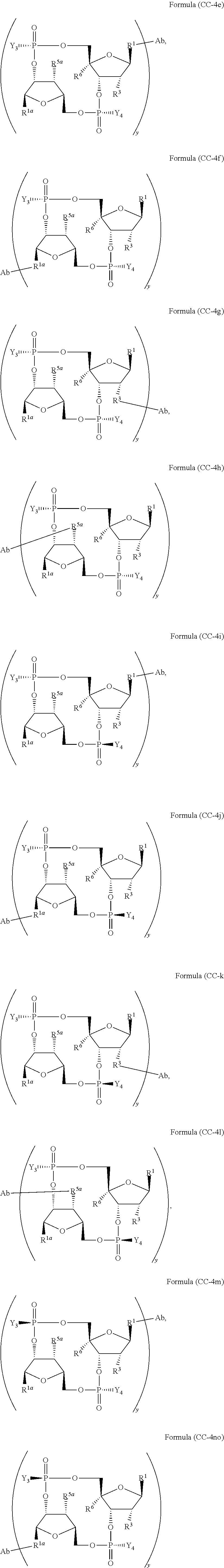

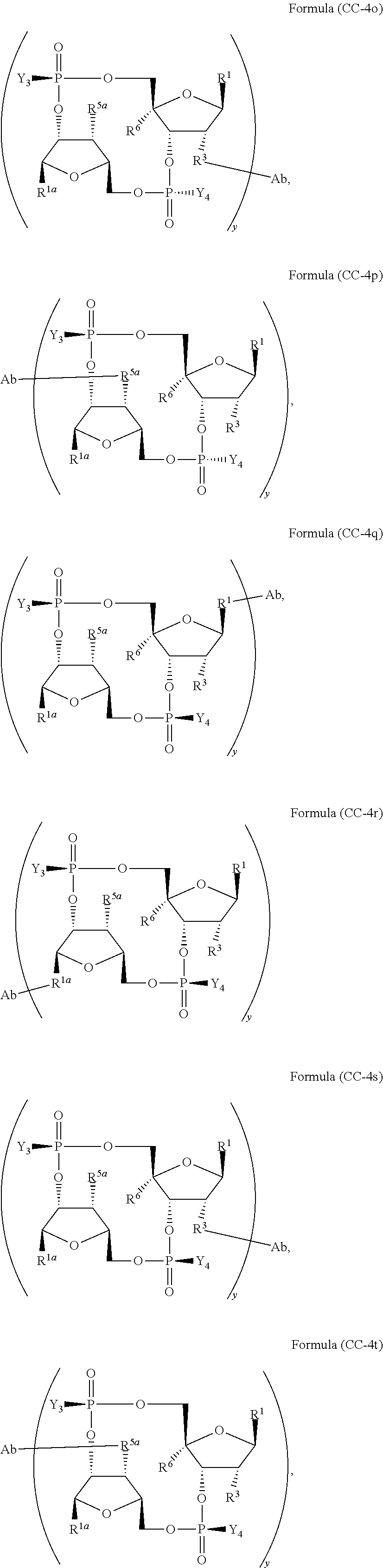

wherein: Ab, y R¹, R^{1a}, R³, R^{5a}, R⁶ and R^{6a} are as defined in Embodiment 205;
Y₃ is OR⁹, N(R¹⁰)₂, SH or S⁻, and
Y₄ is OR⁹, N(R¹⁰)₂, SH or S⁻.

### Embodiment 214

A DC-SIGN immunoconjugate selected from:

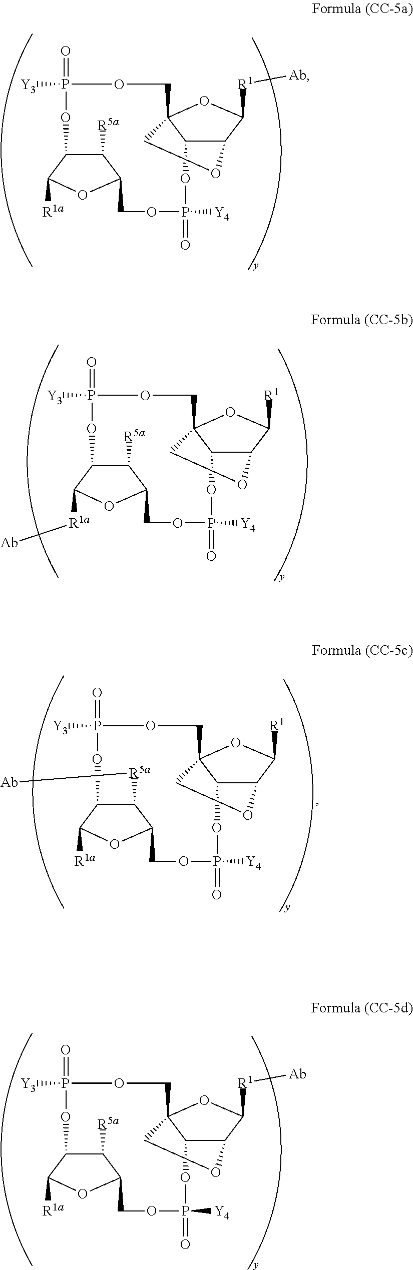

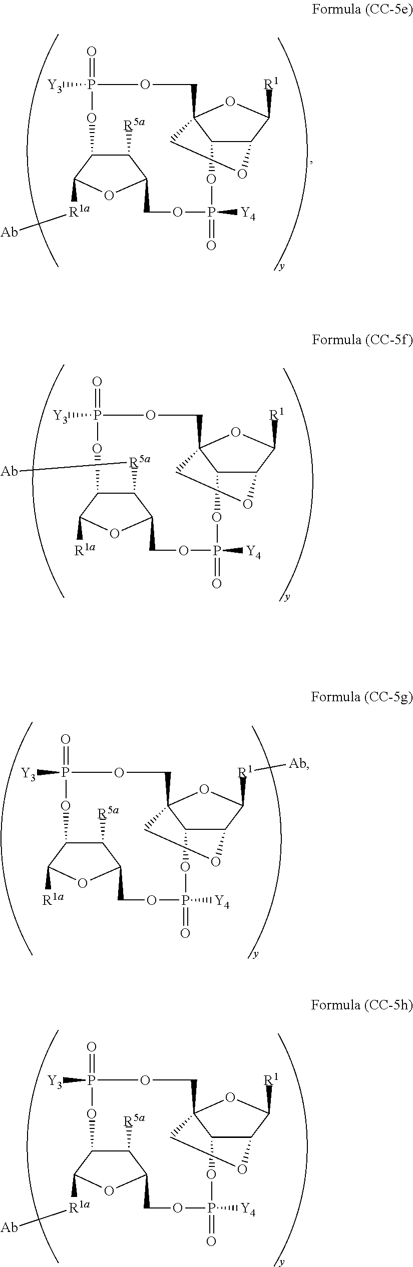

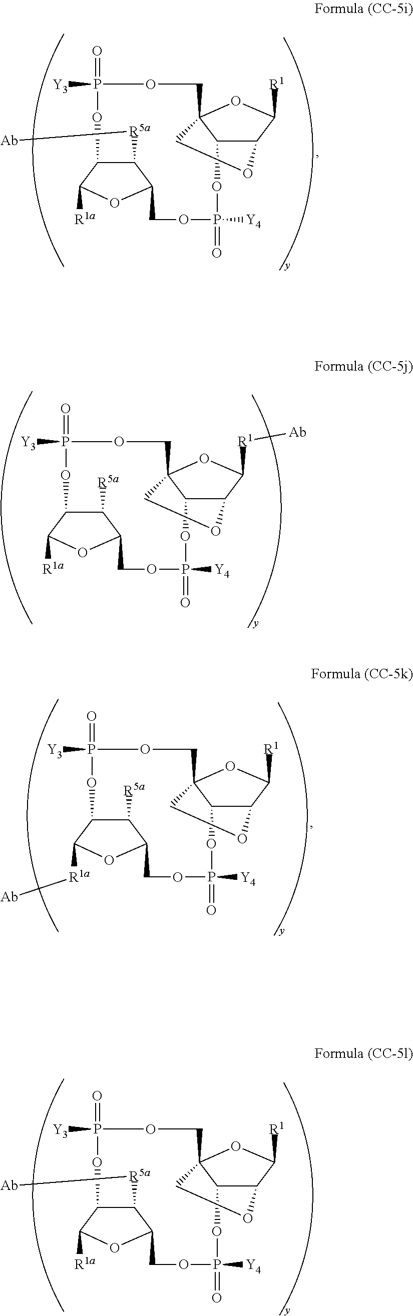

wherein: Ab, y, R¹, R¹, R^{5a} and R^{6a} are as defined in Embodiment 205;
Y₃ is OR⁹, N(R¹⁰)₂, SH or S⁻, and
Y₄ is OR⁹, N(R¹⁰)₂, SH or S⁻.

### Embodiment 215

A DC-SIGN immunoconjugate selected from:

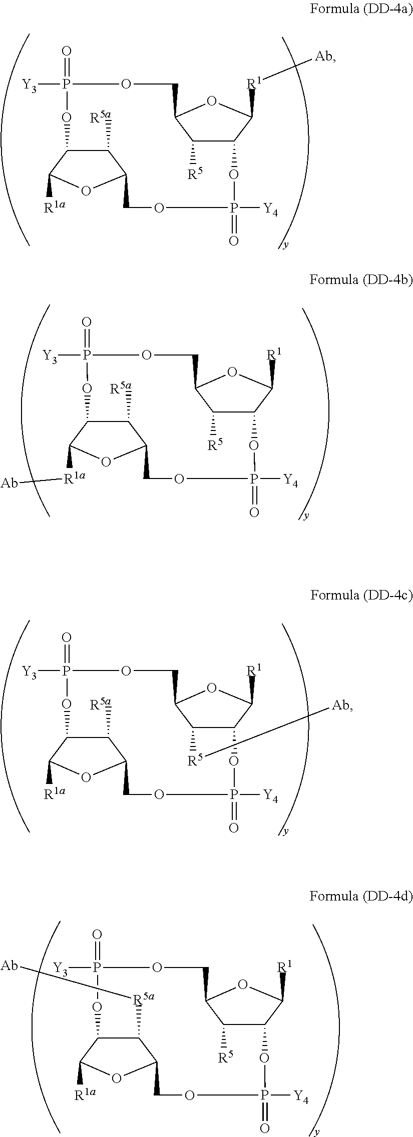

wherein: Ab, y R¹, R^{1a}, R⁵, R^{5a}, Y₃ and Y₄ are as defined in Embodiment 205.

### Embodiment 216

A DC-SIGN immunoconjugate selected from:

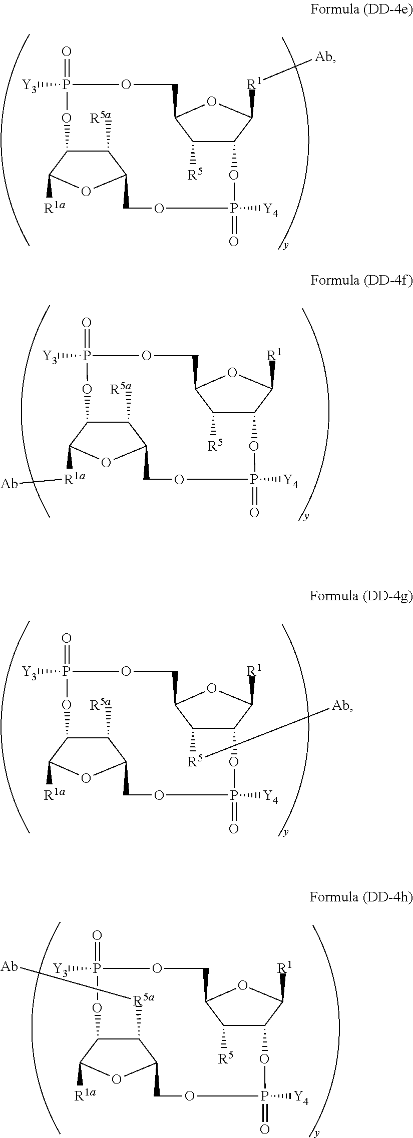

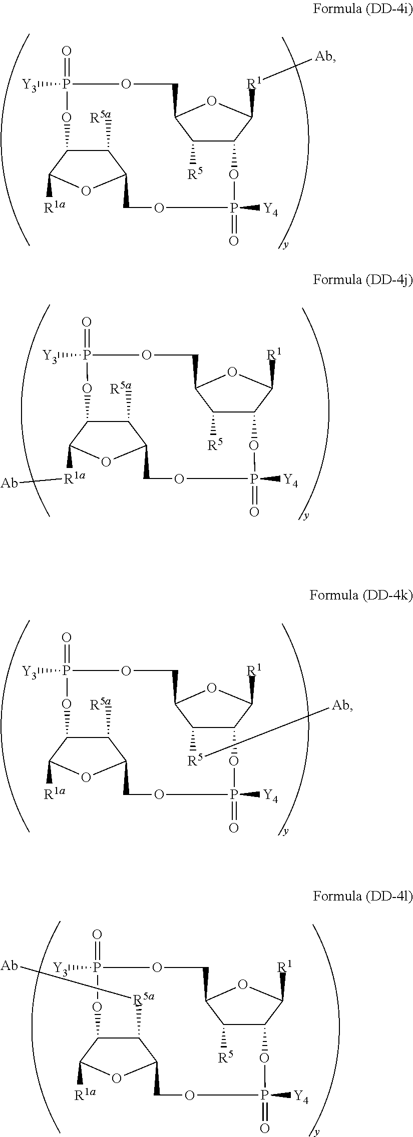

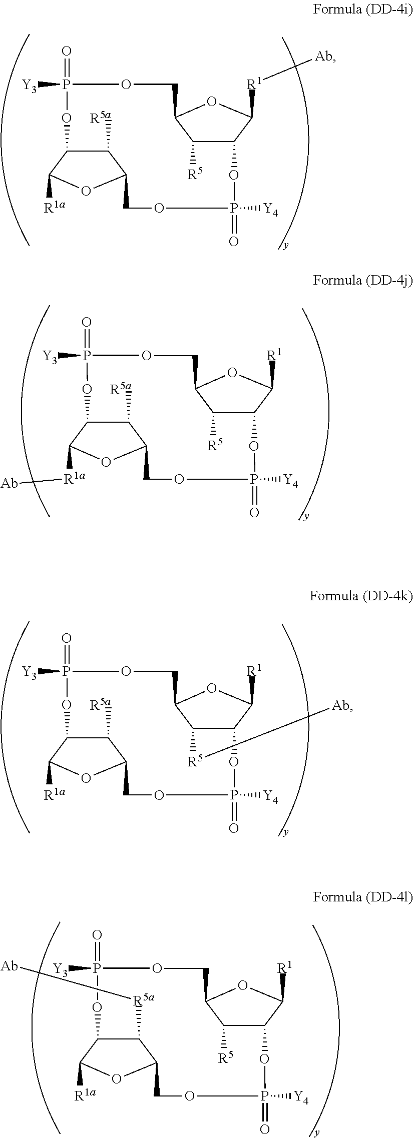

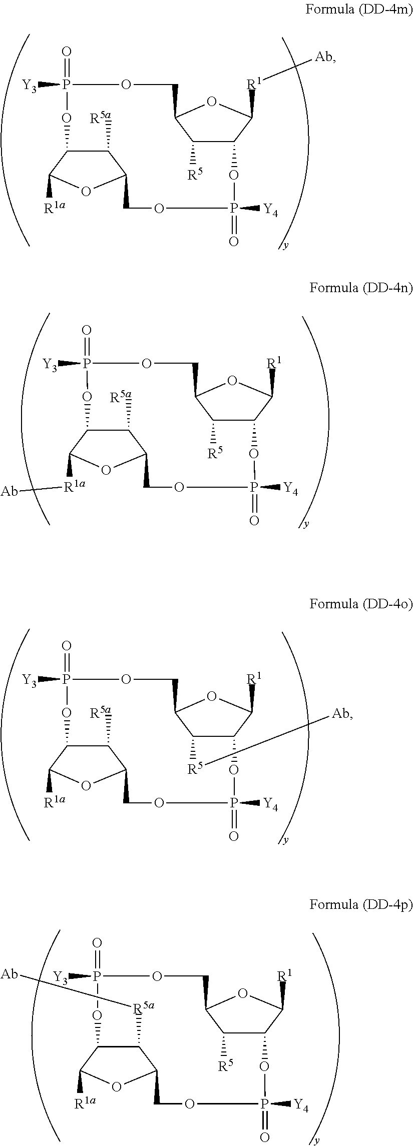

wherein: Ab, y, R¹, R^{1a}, R⁵ and R^{5a} are as defined in Embodiment 205;
Y₃ is OR⁹, N(R¹⁰)₂, SH or S⁻, and
Y₄ is OR⁹, N(R¹⁰)₂, SH or S⁻.

### Embodiment 217

A DC-SIGN immunoconjugate selected from:

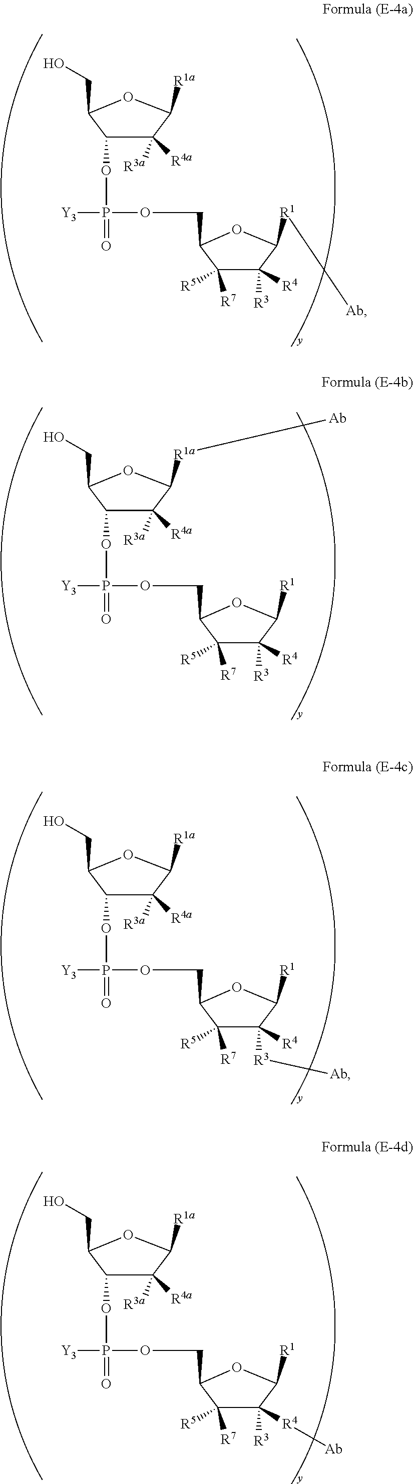

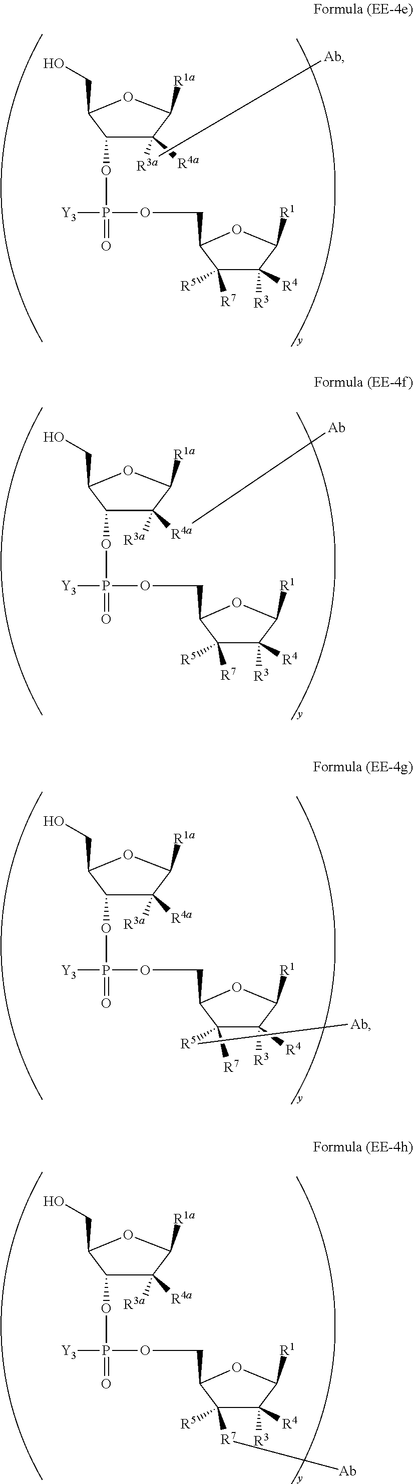

wherein: Ab, y, R¹, R^{1a}, R³, R^{3a}, R⁴, R^{4a}, R⁵, R⁷, R and Y₃ are as defined in Embodiment 205.

### Embodiment 218

A DC-SIGN immunoconjugate selected from:

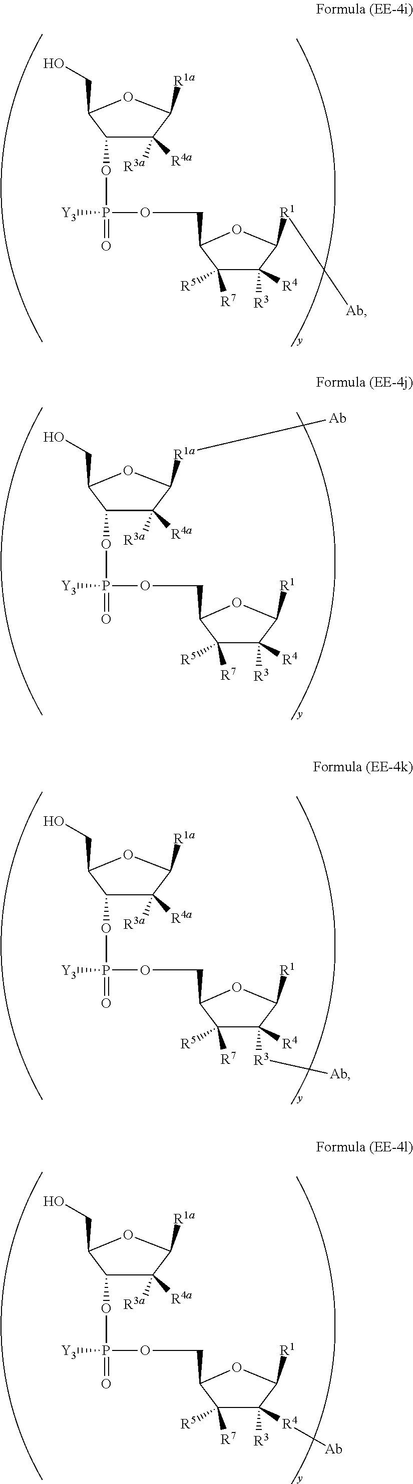

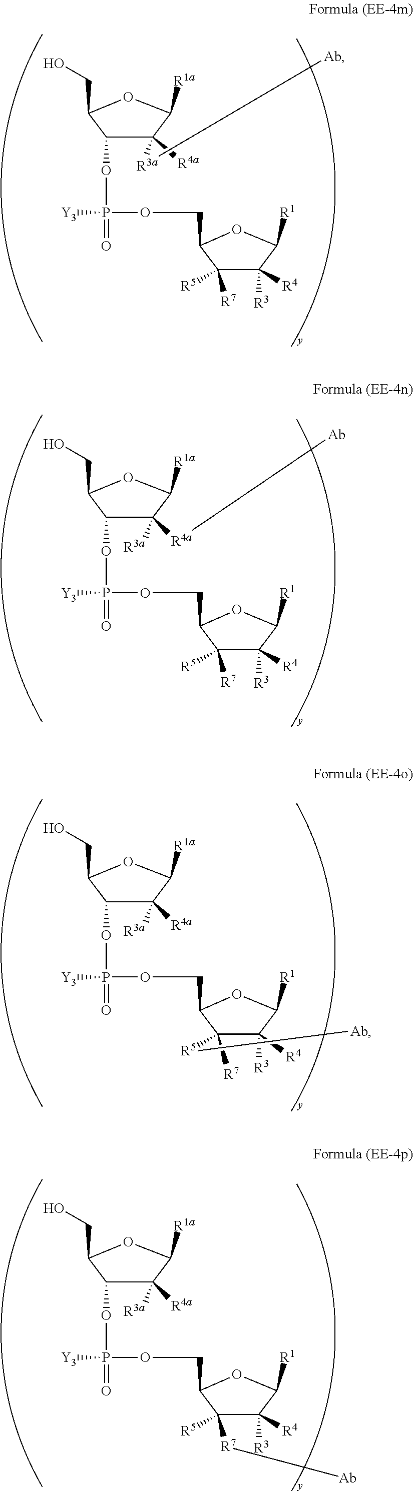

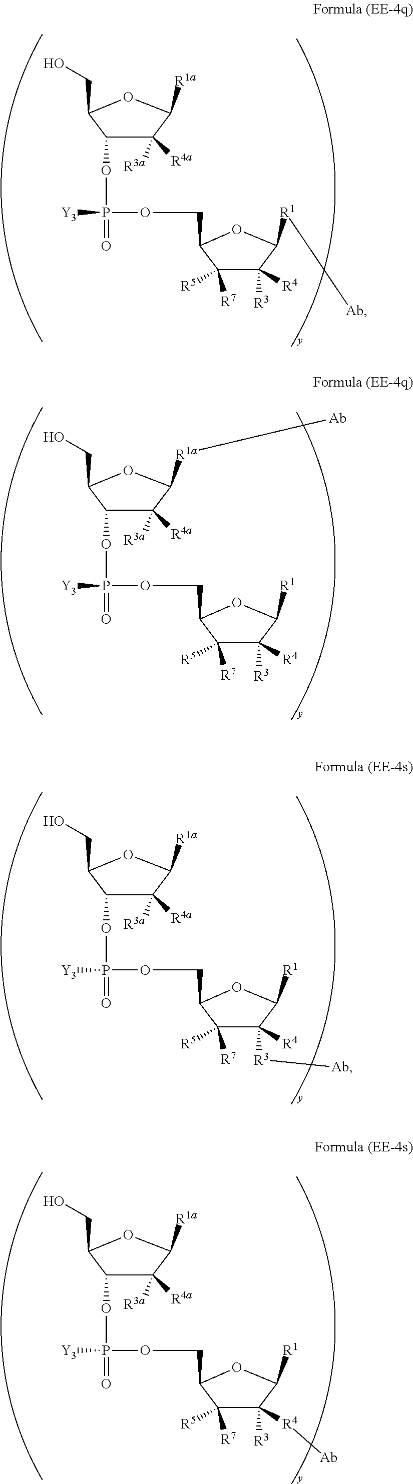

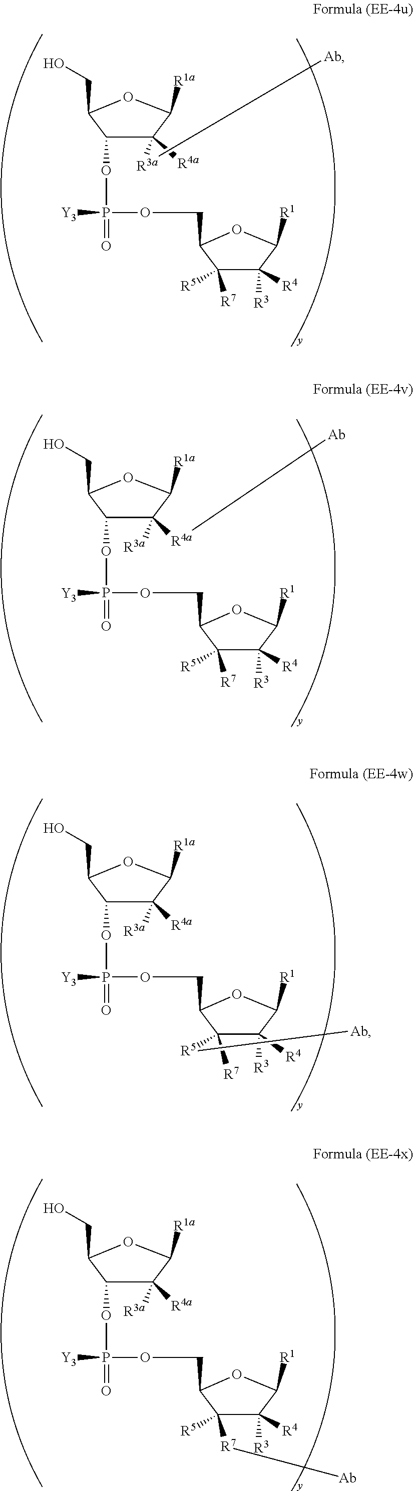

wherein: Ab, y, R¹, R¹, R³, R^{3a}, R⁴, R^{4a}, R⁵, R⁷ and Y₃ are as defined in Embodiment 205;
and Y₃ is OR⁹, N(R¹⁰)₂, SH or S⁻.

### Embodiment 219

A DC-SIGN immunoconjugate selected from:

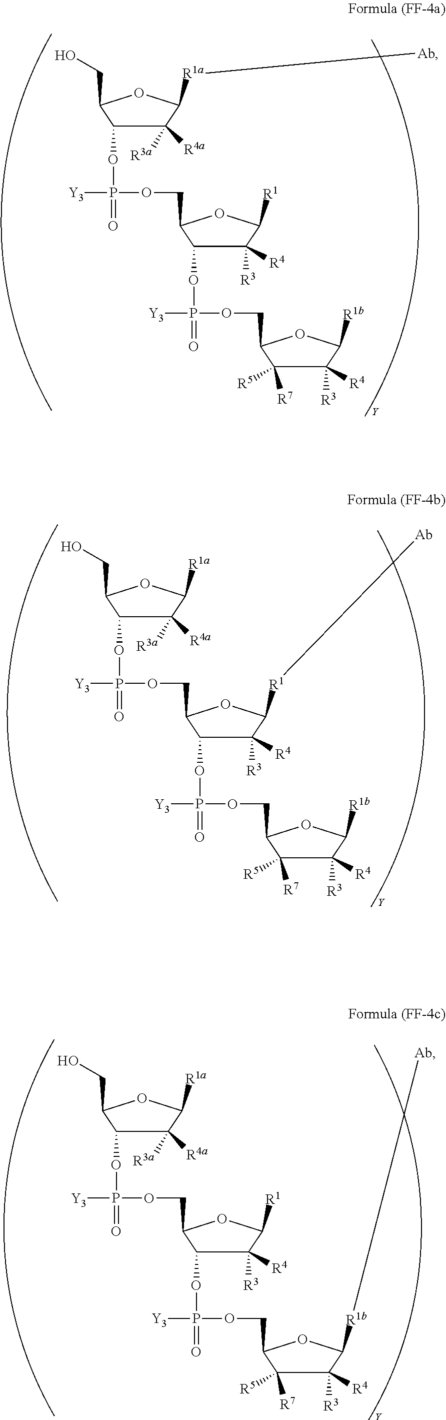

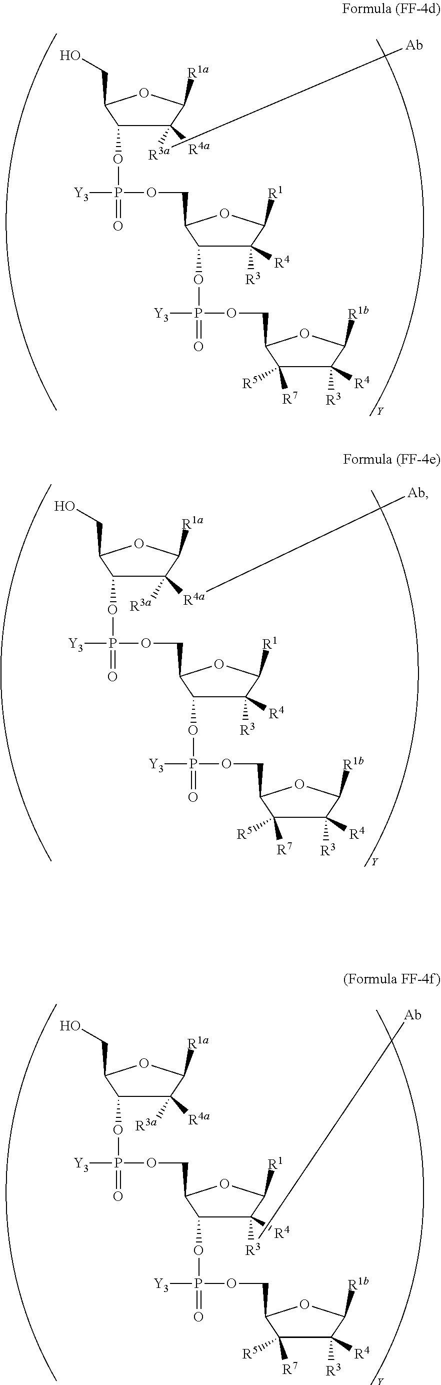

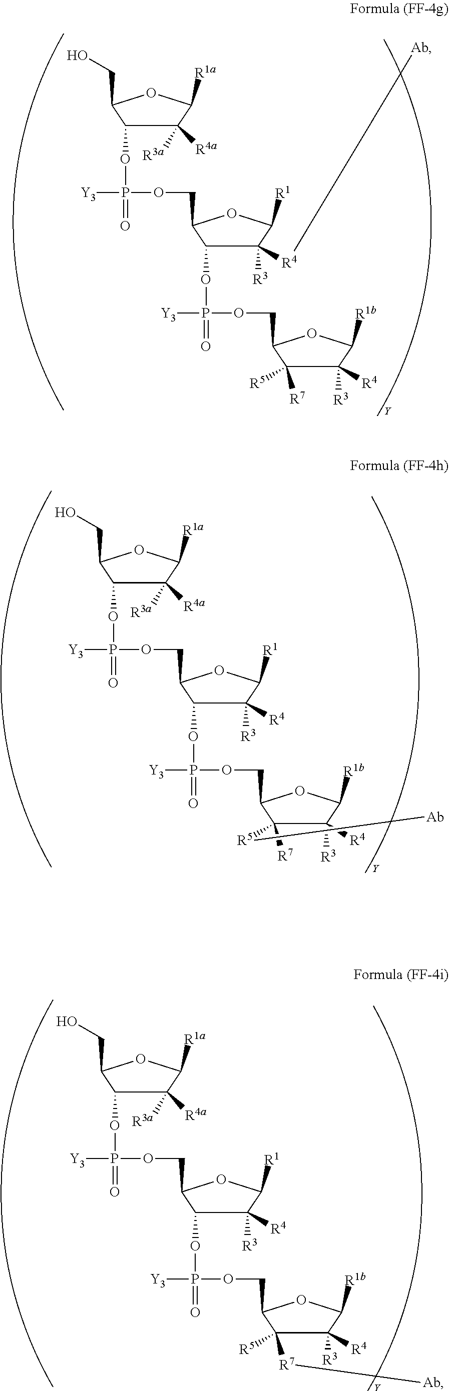

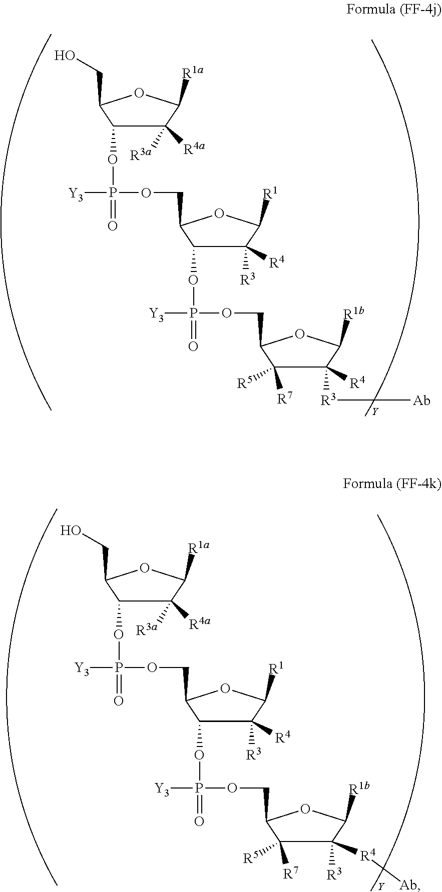

wherein: Ab, y, R¹, R^{1a}, R^{1b}, R³, R^{3a}, R⁴, R^{4a}, R⁵, R⁷ and Y₃ are as defined in Embodiment 205,

### Embodiment 220

A DC-SIGN immunoconjugate selected from:

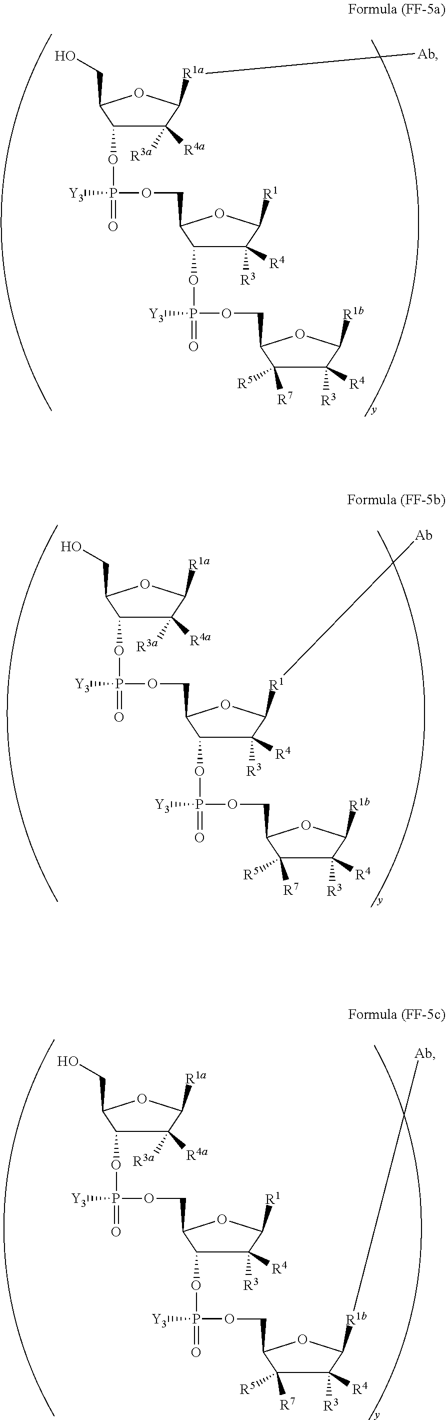

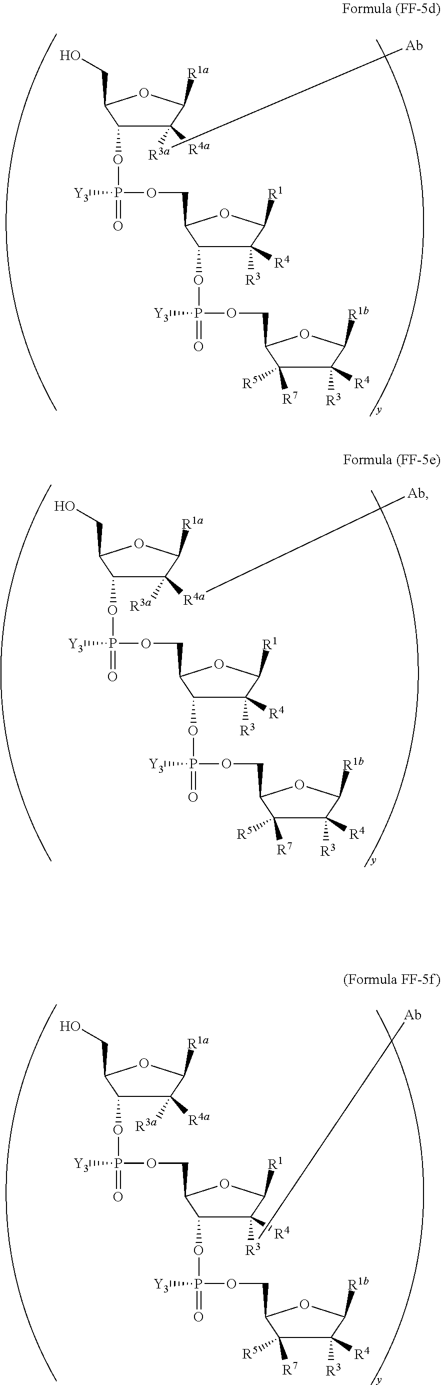

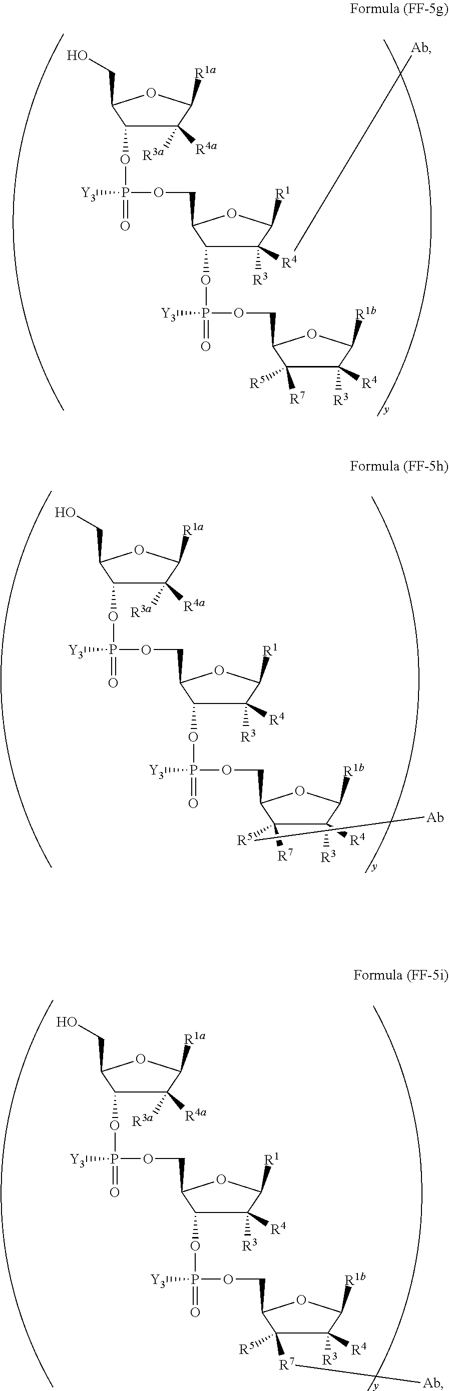

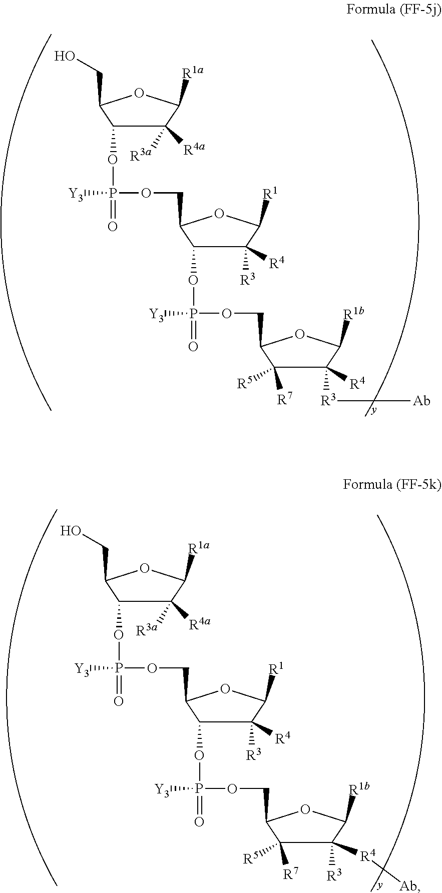

wherein: Ab, y, R¹, R^{1a}, R^{1b}, R³, R^{3a}, R⁴, R^{4a}, R⁵ and R⁷ are as defined in Embodiment 205, and
each Y₃ is independently selected from OR¹⁰, N(R¹⁰)₂, SH and S⁻.

### Embodiment 221

A DC-SIGN immunoconjugate selected from:

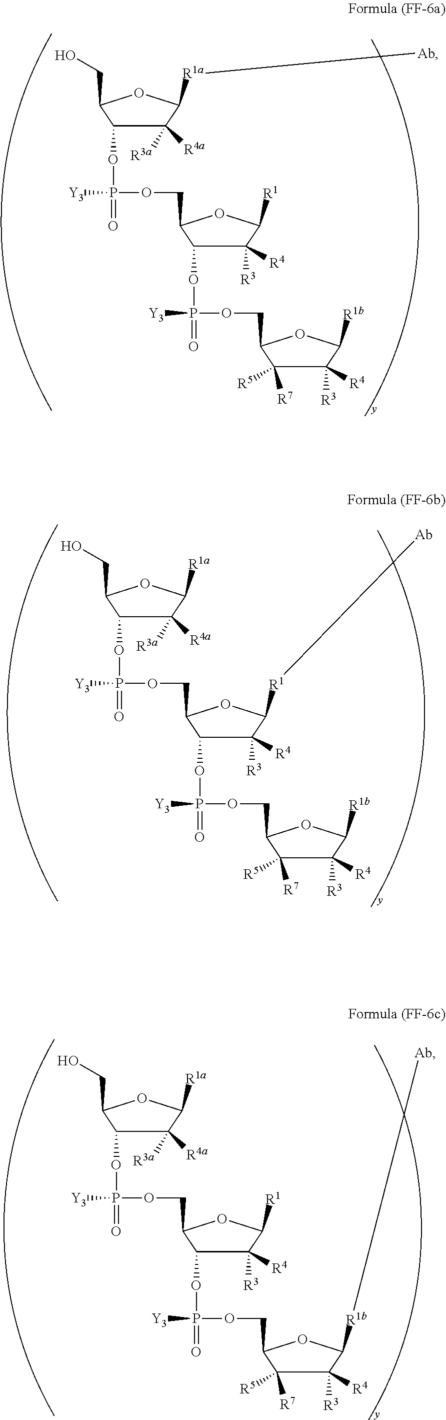

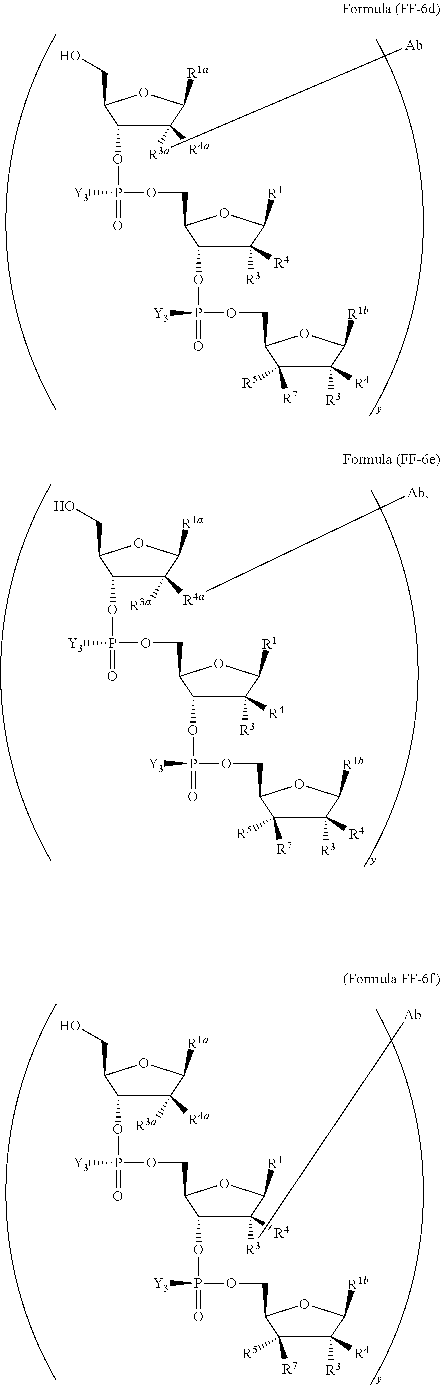

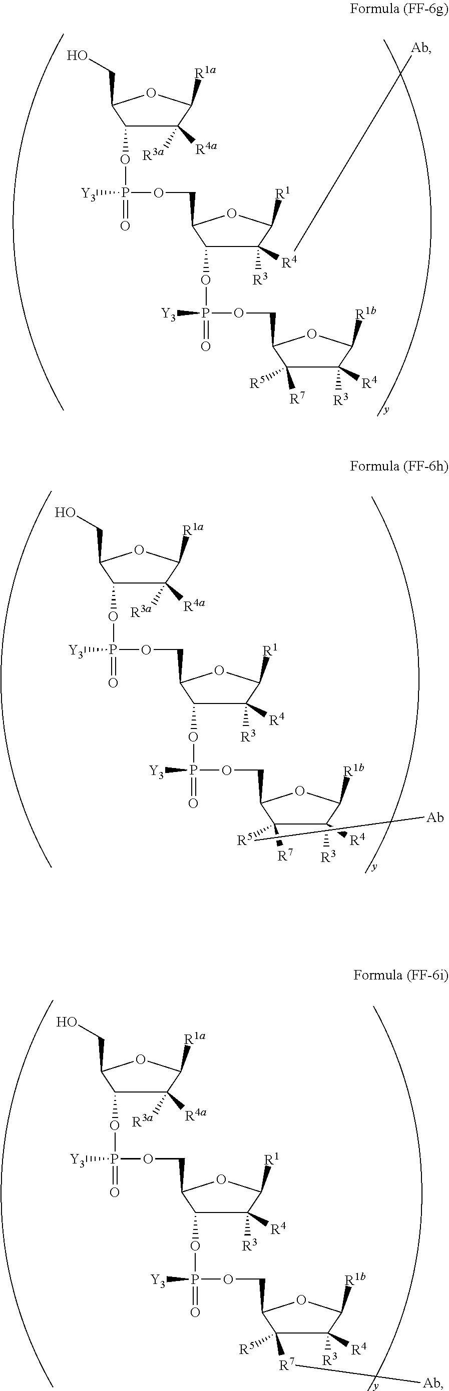

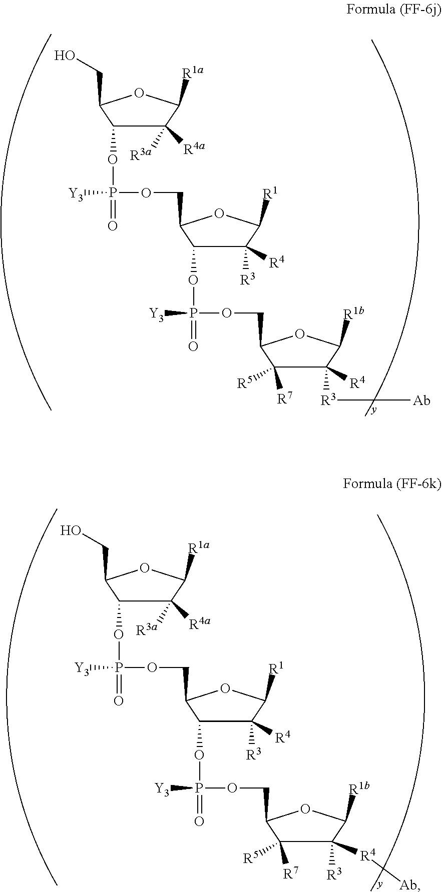

wherein: Ab, y, R¹, R¹, R^{1b}, R³, R^{3a}, R⁴, R^{4a}, R⁵ and R⁷ are as defined in Embodiment 205, and
each Y₃ is independently selected from OR¹⁰, N(R¹⁰)₂, SH and S⁻.

### Embodiment 222

A DC-SIGN immunoconjugate selected from:

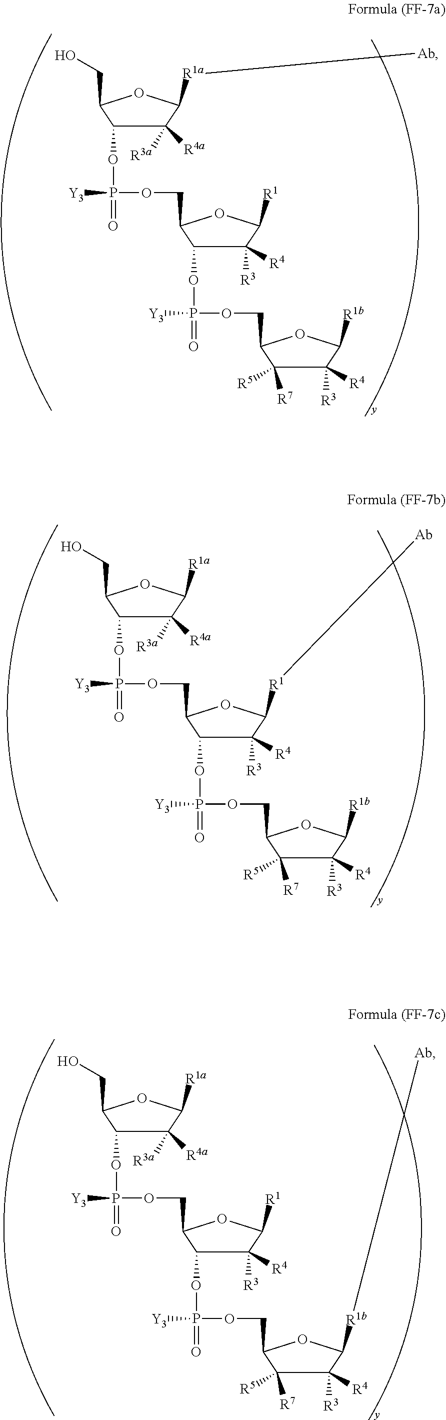

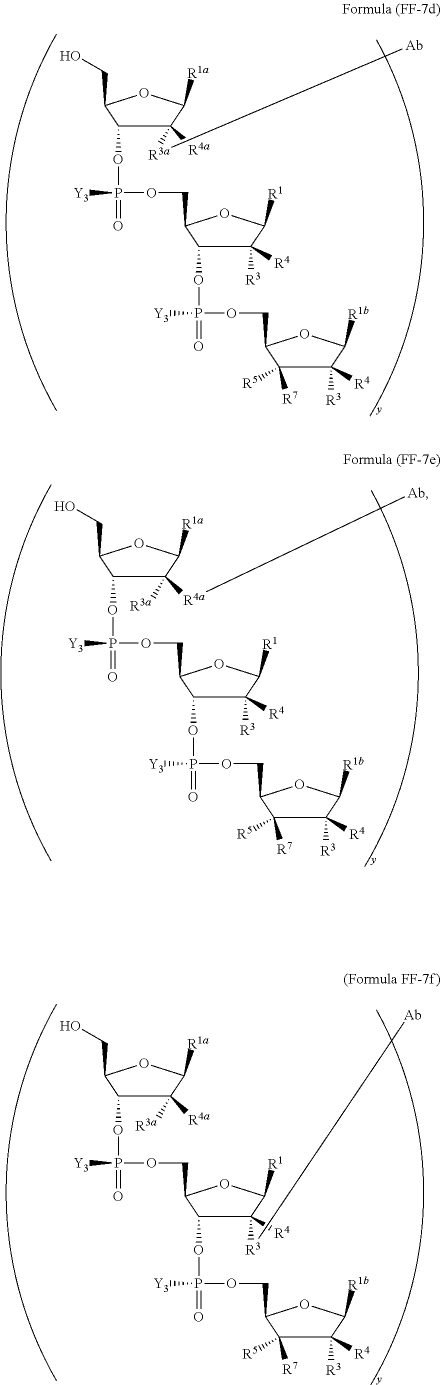

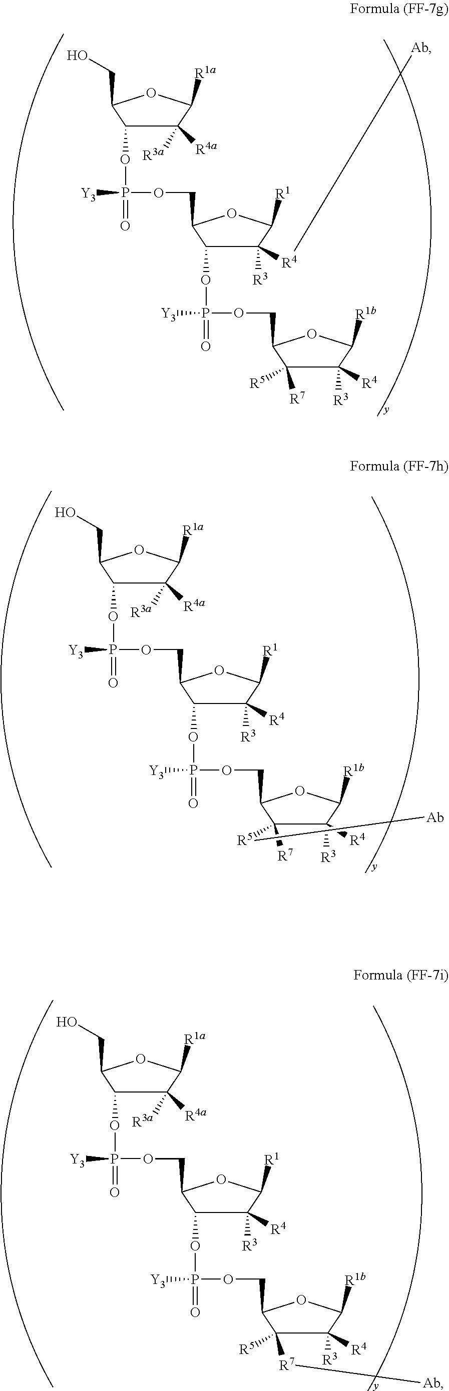

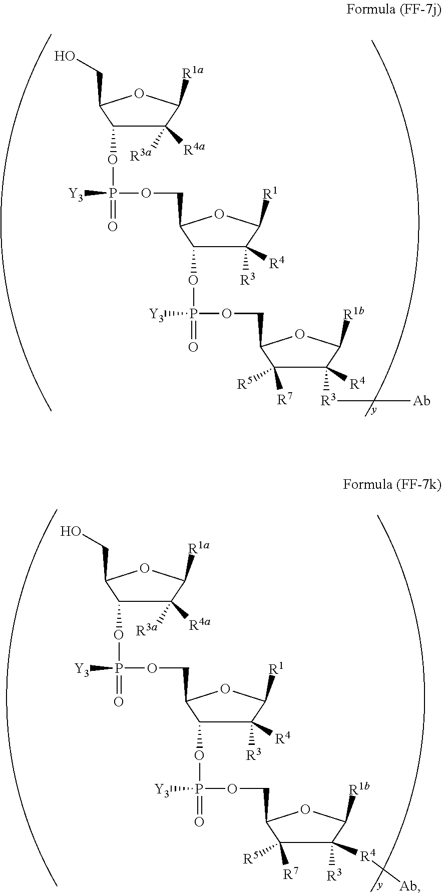

wherein: Ab, y, R¹, R^{1a}, R^{1b}, R³, R^{3a}, R⁴, R^{4a}, R⁵ and R⁷ are as defined in Embodiment 205, and
each Y₃ is independently selected from OR¹⁰, N(R¹⁰)₂, SH and S⁻.

### Embodiment 223

A DC-SIGN immunoconjugate selected from:

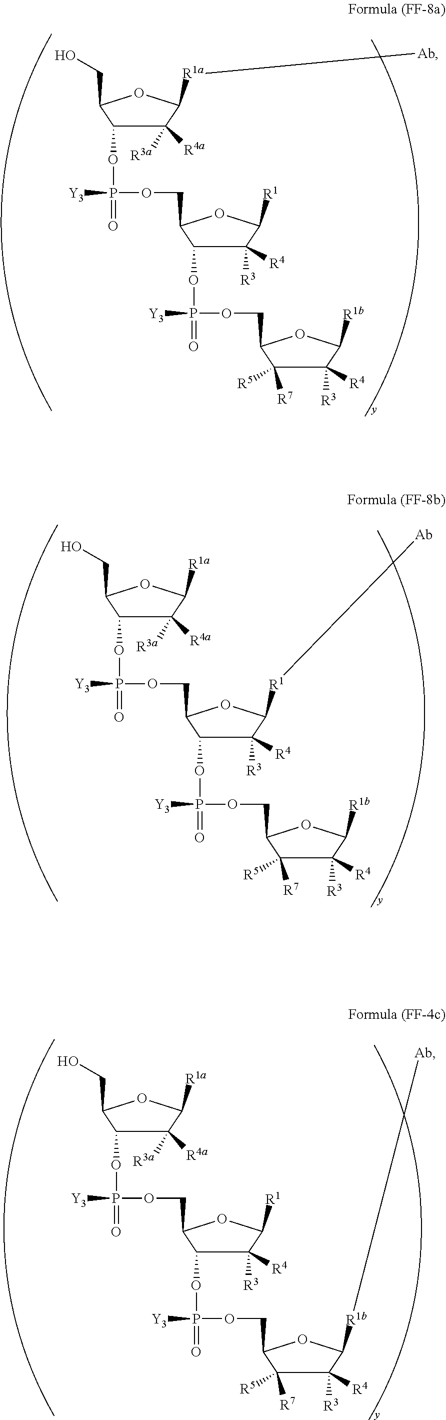

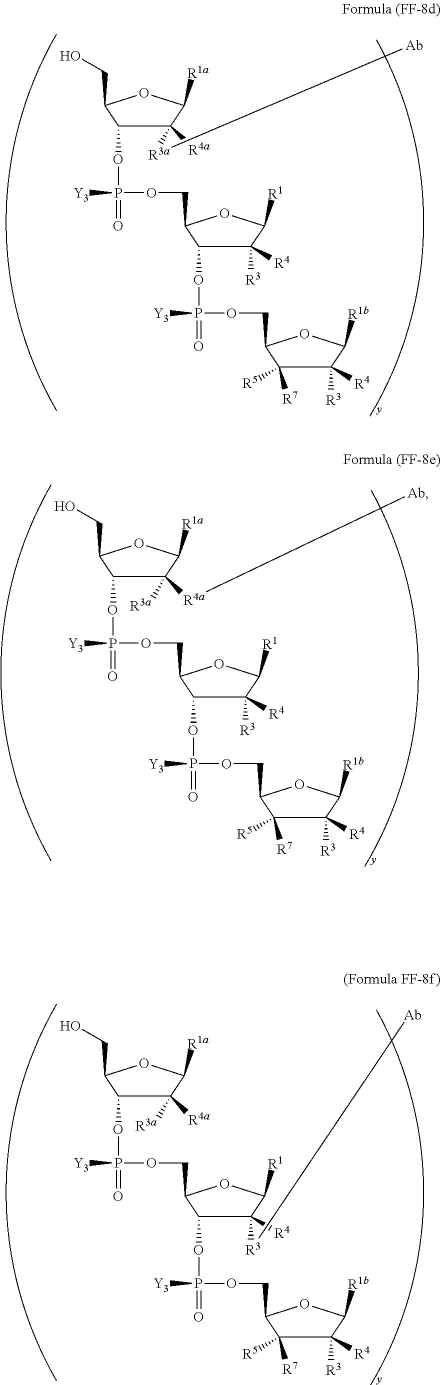

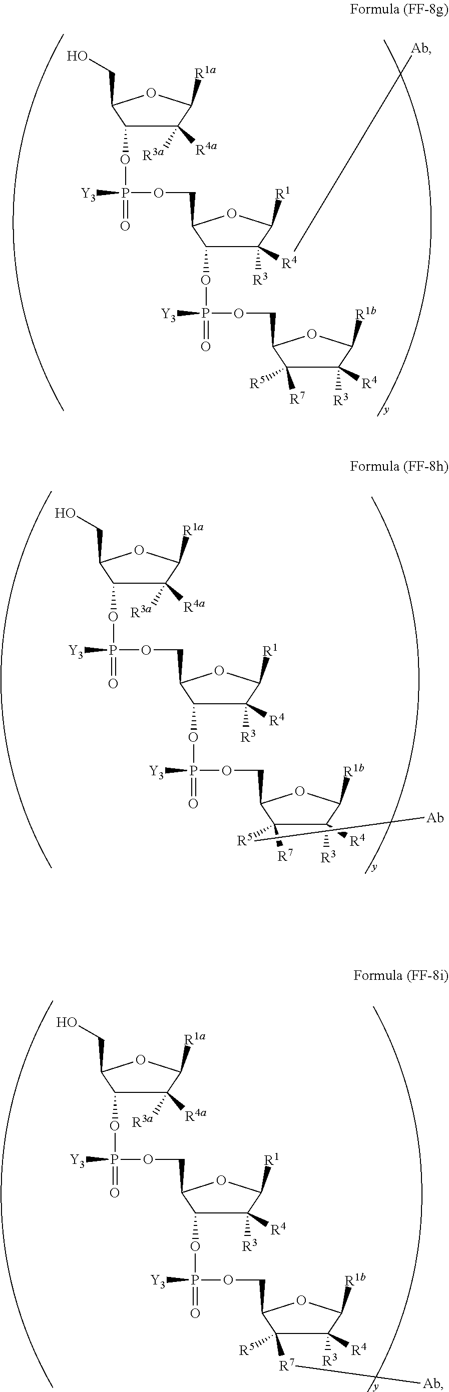

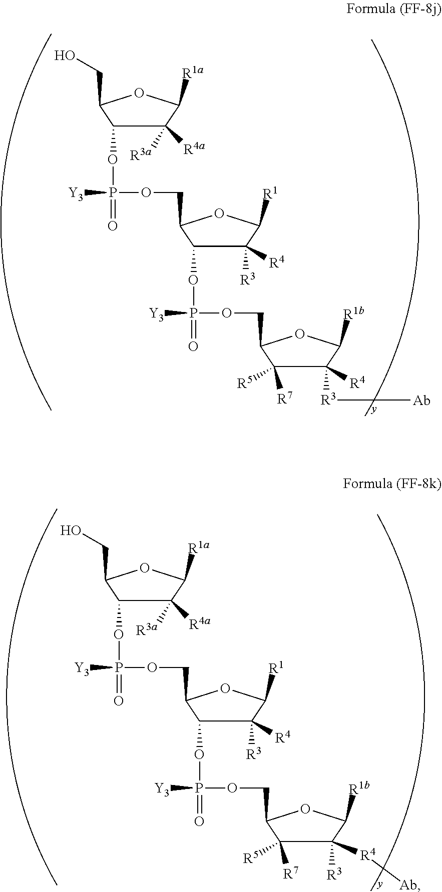

wherein: Ab, y, R¹, R^{1a}, Rb, R³, R^{3a}, R⁴, R^{4a}, R⁵ and R⁷ are as defined in Embodiment 205, and
each Y₃ is independently selected from OR⁰, N(R¹⁰)₂, SH and S⁻.

### Embodiment 224

The DC-SIGN immunoconjugate of any one of Embodiments 188 to 223, wherein R¹ is

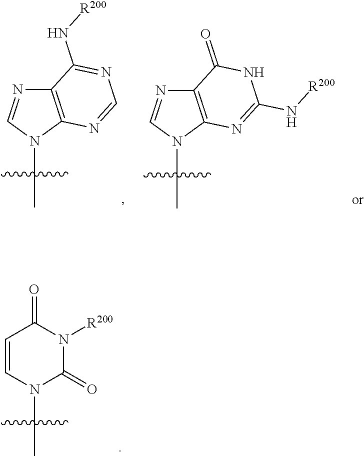

### Embodiment 225

The DC-SIGN immunoconjugate of any one of Embodiments 188 to 223, wherein R^{1a} is

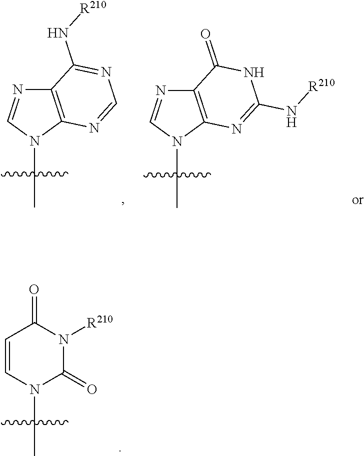

### Embodiment 226

The DC-SIGN immunoconjugate of any one of Embodiments 188 to 223, wherein R^{1b} is

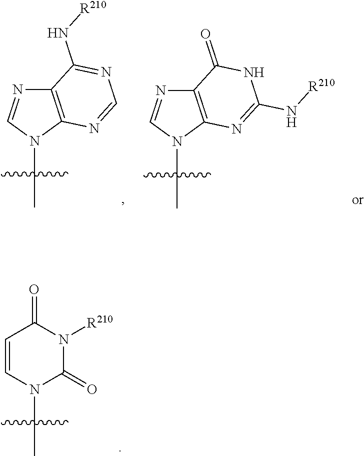

### Embodiment 227

The DC-SIGN immunoconjugate of any one of Embodiments 188 to 223, wherein R^{1a} is

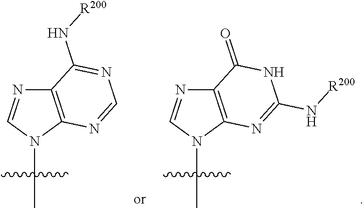

### Embodiment 228

The DC-SIGN immunoconjugate of any one of Embodiments 188 to 223, wherein R^{1a} is

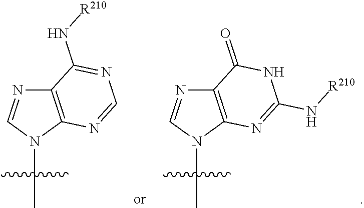

### Embodiment 229

The DC-SIGN immunoconjugate of any one of Embodiments 188 to 223, wherein R^{1b} is

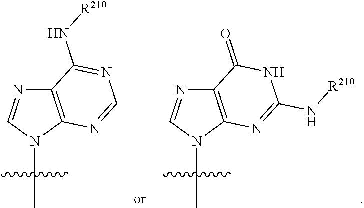

### Embodiment 230

The compound of any one of Embodiments 188 to 223, wherein R¹ is

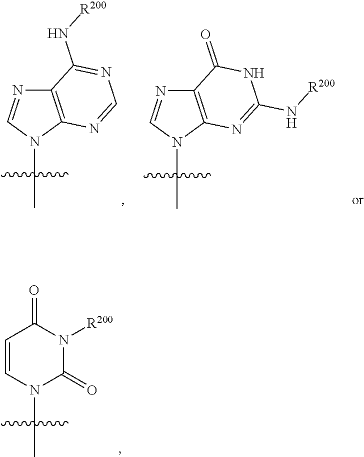

wherein R²⁰⁰ is -L₁R¹¹⁵.

### Embodiment 231

The compound of any one of Embodiments 188 to 223, wherein R^{1a} is

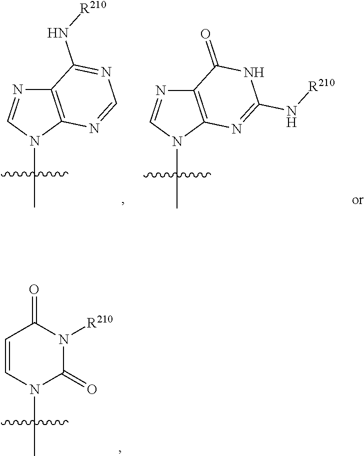

wherein R²¹⁰ is -L₁R¹¹⁵.

### Embodiment 232

The compound of any one of Embodiments 188 to 223, wherein R^{1b} is is

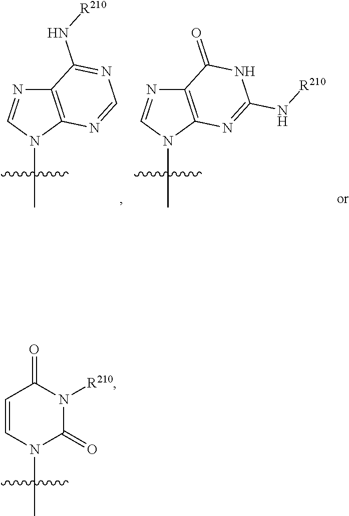

wherein R²¹⁰ is -L₁R¹¹⁵.

### Embodiment 233

The compound of any one of Embodiments 188 to 223, wherein R¹ is

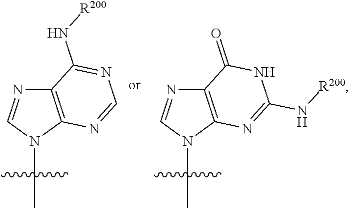

wherein R²⁰⁰ is -L₁R¹¹⁵.

### Embodiment 234

The compound of any one of Embodiments 188 to 223, wherein R^{1a} is

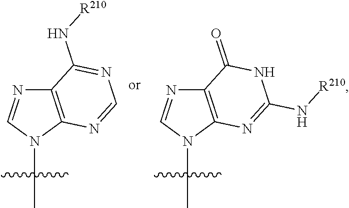

wherein R²¹⁰ is -L₁R¹¹⁵.

### Embodiment 235

The compound of any one of Embodiments 188 to 223, wherein R^{1b} is

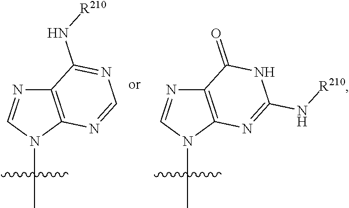

wherein R²¹⁰ is -L₁R¹¹⁵.

### Embodiment 236

The DC-SIGN immunoconjugate of any one of Embodiments 188 to 223, wherein R¹ is

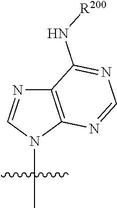

### and R^{1a} is

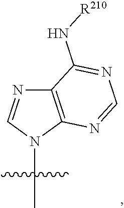

wherein R²⁰⁰ is L₁R¹¹⁵ and R²¹⁰ is H.

### Embodiment 237

The DC-SIGN immunoconjugate of any one of Embodiments 188 to 223, wherein R¹ is

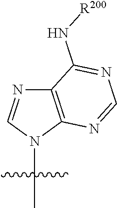

### and R^{1a} is

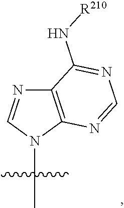

wherein R²⁰⁰ is H and R²¹⁰ is L₁R¹¹⁵.

### Embodiment 238

The DC-SIGN immunoconjugate of any one of Embodiments 188 to 223, wherein R¹ is

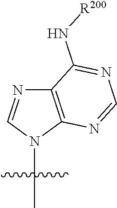

### and R^{1a} is

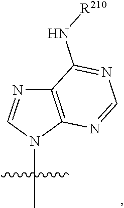

wherein R²⁰⁰ is L₁R¹¹⁵ and R²¹⁰ is L₁R¹¹⁵

### Embodiment 239

The DC-SIGN immunoconjugate of any one of Embodiments 188 to 223, wherein R¹ is

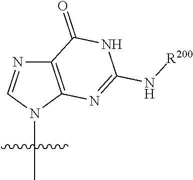

### and R^{1a} is

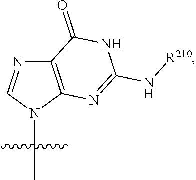

wherein R²⁰⁰ is L₁R¹¹⁵ and R²¹⁰ is H.

### Embodiment 240

The DC-SIGN immunoconjugate of any one of Embodiments 188 to 223, wherein R¹ is

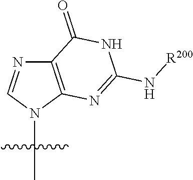

### and R^{1a} is

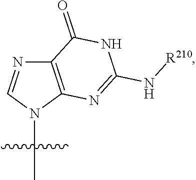

wherein R²⁰⁰ is H and R²¹⁰ is L₁R¹¹⁵.

### Embodiment 241

The DC-SIGN immunoconjugate of any one of Embodiments 188 to 223, wherein R¹ is

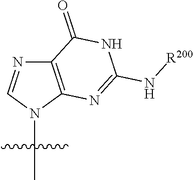

### and R^{1a} is

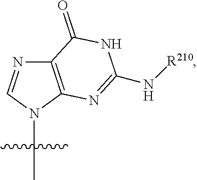

wherein R²⁰⁰ is L₁R¹¹⁵ and R²¹⁰ is L₁R¹¹⁵.

### Embodiment 242

The DC-SIGN immunoconjugate of any one of Embodiments 188 to 223, wherein R¹ is

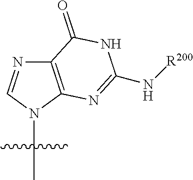

### and R^{1a} is

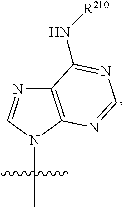

wherein R²⁰⁰ is H and R²¹⁰ is L₁R¹¹⁵.

### Embodiment 243

The DC-SIGN immunoconjugate of any one of Embodiments 188 to 223, wherein R¹ is

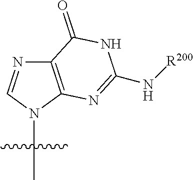

### and R^{1a} is

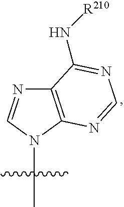

wherein R²⁰⁰ is L₁R¹⁵ and R²¹⁰ is H.

### Embodiment 244

The DC-SIGN immunoconjugate of any one of Embodiments 188 to 223, wherein R¹ is

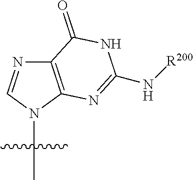

### and R^{1a} is

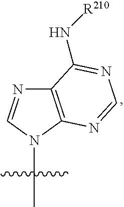

wherein R²⁰⁰ is L₁R¹¹⁵ and R²¹⁰ is L₁R¹¹⁵.

### Embodiment 245

The DC-SIGN immunoconjugate of any one of Embodiments 188 to 223, wherein R¹ is

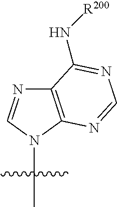

### and R^{1a} is

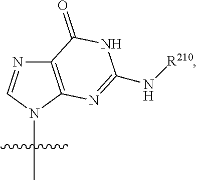

wherein R²⁰⁰ is H and R²¹⁰ is L₁R¹¹⁵.

### Embodiment 246

The DC-SIGN immunoconjugate of any one of Embodiments 188 to 223, wherein R¹ is

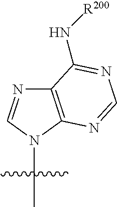

### and R^{1a} is

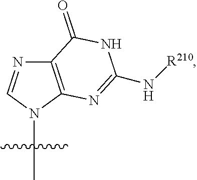

wherein R²⁰⁰ is L₁R¹¹⁵ and R²¹⁰ is H.

### Embodiment 247

The DC-SIGN immunoconjugate of any one of Embodiments 188 to 223, wherein R¹ is

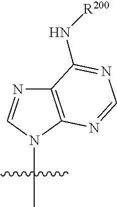

### and R^{1a} is

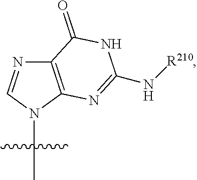

wherein R²⁰⁰ is L₁R¹¹⁵ and R²¹⁰ is L₁R¹¹⁵.

### Embodiment 248

The DC-SIGN immunoconjugate of any one of Embodiments 188 to 223, wherein R¹ is

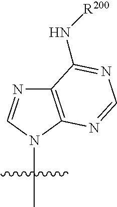

### and R^{1a} is

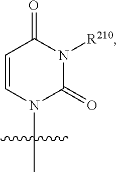

wherein R²⁰⁰ is L₁R¹¹⁵ and R²¹⁰ is H.

### Embodiment 249

The DC-SIGN immunoconjugate of any one of Embodiments 188 to 223, wherein R¹ is

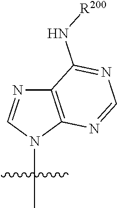

### and R^{1a} is

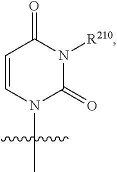

wherein R²⁰⁰ is H and R²¹⁰ is L₁R¹¹⁵.

### Embodiment 250

The DC-SIGN immunoconjugate of any one of Embodiments 188 to 223, wherein R¹ is

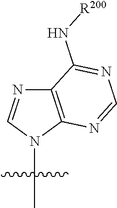

### and R^{1a} is

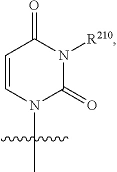

wherein R²⁰⁰ is L₁R¹¹⁵ and R²¹⁰ is L₁R¹¹⁵.

### Embodiment 251

The DC-SIGN immunoconjugate of any one of Embodiments 188 to 223, wherein R¹ is

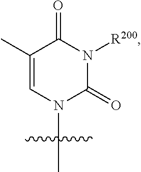

### R^{1b} is

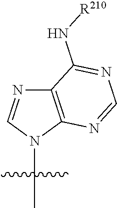

### and R^{1a} is

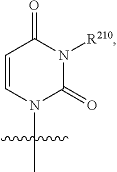

wherein R²⁰⁰ is L₁R¹¹⁵ and each R²¹⁰ is H.

### Embodiment 252

The DC-SIGN immunoconjugate of any one of Embodiments 188 to 223, wherein R¹ is

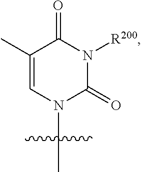

### R^{1b} is

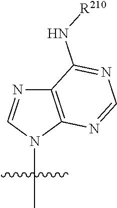

### and R^{1a} is

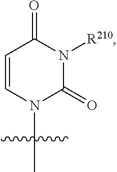

wherein R²⁰⁰ is H, R²¹⁰ of R^{1b} is L₁R¹¹⁵ and R²¹ of R^{1a} is H.

### Embodiment 253

The DC-SIGN immunoconjugate of any one of Embodiments 188 to 223, wherein R¹ is

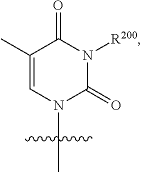

### R^{1b} is

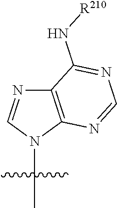

### and R^{1a} is

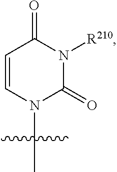

wherein R²⁰⁰ is H, R²¹⁰ of Rib is H and R²¹⁰ of R^{1a} is L₁R¹¹⁵.

### Embodiment 254

The DC-SIGN immunoconjugate of any one of Embodiments 188 to 223, wherein R¹ is

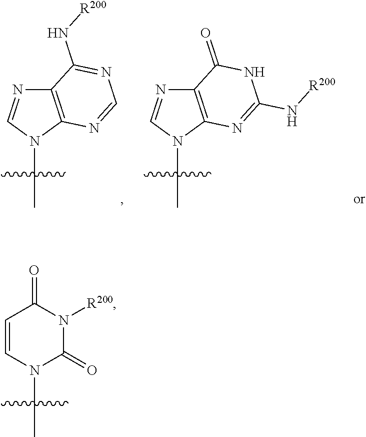

wherein R²⁰⁰ is H and one of R³, R^{3a}, R⁵ or R^{5a} is —OL₁R¹¹⁵.

### Embodiment 255

The DC-SIGN immunoconjugate of any one of Embodiments 188 to 223, wherein R^{1a} is

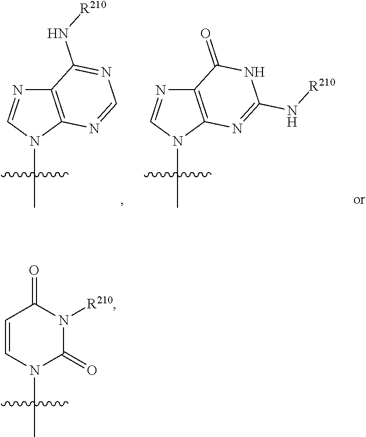

wherein R²¹⁰ is H and one of R³, R^{3a}, R⁵ or R^{5a} is —OL₁R¹¹⁵.

### Embodiment 256

The DC-SIGN immunoconjugate of any one of Embodiments 188 to 223, wherein R^{1b} is is

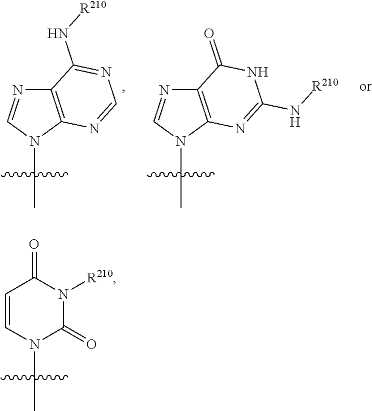

wherein R²¹⁰ is H and one of R³, R^{3a}, R⁵ or R^{5a} is —OL₁R¹¹⁵.

### Embodiment 257

The DC-SIGN immunoconjugate of any one of Embodiments 188 to 223, wherein R¹ is

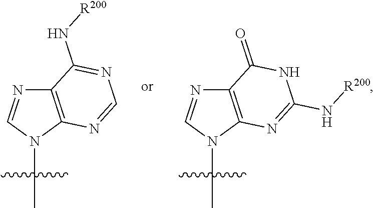

wherein R²⁰⁰ is H and one of R³, R^{3a}, R⁵ or R^{5a} is —OL₁R¹¹⁵.

### Embodiment 258

The DC-SIGN immunoconjugate of any one of Embodiments 188 to 223, wherein R^{1a} is

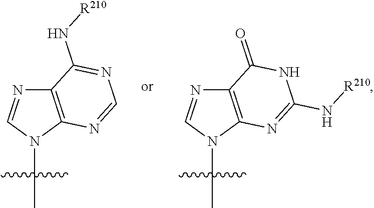

wherein R²¹⁰ is is H and one of R³, R^{3a}, R⁵ or R^{5a} is —OL₁R¹¹⁵.

### Embodiment 259

The DC-SIGN immunoconjugate of any one of Embodiments 188 to 223, wherein R^{1b} is

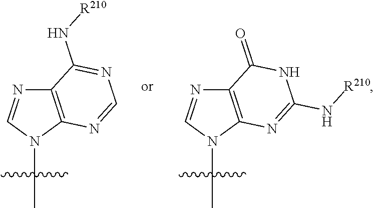

wherein R²¹⁰ is is H and one of R³, R^{3a}, R⁵ or R^{5a} is —OL₁R¹¹⁵.

### Embodiment 260

The DC-SIGN immunoconjugate of any one of Embodiments 188 to 223, wherein
R¹ is

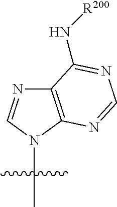

### and R^{1a} is

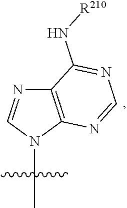

wherein R²⁰⁰ is H, R²¹⁰ is H and one of R³, R^{3a}, R⁵ or R^{5a} is —OL₁R¹¹⁵

### Embodiment 261

The DC-SIGN immunoconjugate of any one of Embodiments 188 to 223, wherein R¹ is

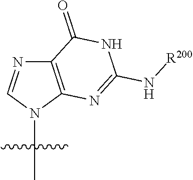

### and R^{1a} is

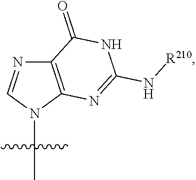

wherein R²⁰⁰ is H, R²¹⁰ is H and one of R³, R^{3a}, R⁵ or R^{5a} is —OL₁R¹¹⁵.

### Embodiment 262

The DC-SIGN immunoconjugate of any one of Embodiments 188 to 223, wherein R¹ is

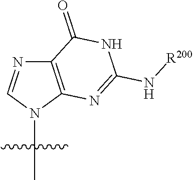

### and R^{1a} is

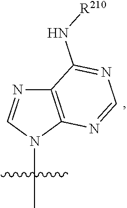

wherein R²⁰⁰ is H, R²¹⁰ is H and one of R³, R^{3a}, R⁵ or R^{5a} is —OL₁R¹¹⁵.

### Embodiment 263

The DC-SIGN immunoconjugate of any one of Embodiments 188 to 223, wherein R¹ is

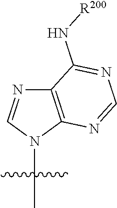

### and R^{1a} is

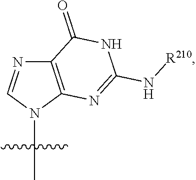

wherein R²⁰⁰ is H, R²¹⁰ is H and one of R³, R^{3a}, R⁵ or R^{5a} is —OL₁R¹¹⁵.

### Embodiment 264

The DC-SIGN immunoconjugate of any one of Embodiments 188 to 223, wherein R¹ is

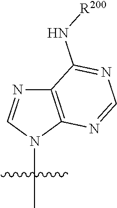

### and R^{1a} is

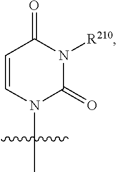

wherein R²⁰⁰ is H, R²¹⁰ is H and one of R³, R^{3a}, R⁵ or R^{5a} is —OL₁R¹¹⁵.

### Embodiment 265

The DC-SIGN immunoconjugate of any one of Embodiments 188 to 223, wherein R¹ is

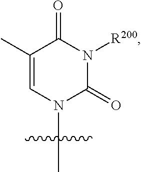

### R^{1b} is

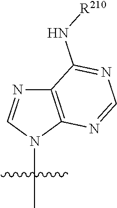

### and R^{1a} is

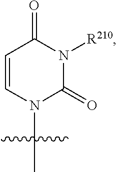

wherein R²⁰⁰ is H, R²¹⁰ is H and one of R³, R^{3a}, R⁵ or R^{5a} is —OL₁R¹¹⁵.

### Embodiment 266

The DC-SIGN immunoconjugate of any one of Embodiments 188 to 266, wherein:
Y₃ is OH, O⁻, SH or S⁻, and
Y₄ is OH, O⁻, SH or S⁻.

### Embodiment 267

The DC-SIGN immunoconjugate of any one of Embodiments 188 to 266, wherein:
Y₃ is OH or O⁻, and
Y₄ is OH or O⁻.

### Embodiment 268

The DC-SIGN immunoconjugate of any one of Embodiments 188 to 266, wherein:
Y₃ is SH or S⁻, and
Y₄ is OH or O⁻.

### Embodiment 269

The DC-SIGN immunoconjugate of any one of Embodiments 188 to 266, wherein:
Y₃ is OH or O⁻, and
Y₄ is SH or S⁻.

### Embodiment 270

The DC-SIGN immunoconjugate of any one of Embodiments 188 to 266, wherein:
Y₃ is SH or S⁻, and
Y₄ is SH or S⁻.

### Embodiment 271

The DC-SIGN immunoconjugate of any one of Embodiments 188 to 253 or Embodiments 267 to 271, wherein: R², R^{2a}, R⁴, R^{4a}, R⁶, R^{6a}, R⁷ and R^{7a} are each H.

### Embodiment 272

The DC-SIGN immunoconjugate of any one of Embodiments 188 to 253 or Embodiments 267 to 271, wherein: R³ is —OH, F or —NH₂.

### Embodiment 273

The DC-SIGN immunoconjugate of any one of Embodiments 188 to 253 or Embodiments 267 to 271 wherein: R³ is —OH or F.

### Embodiment 274

The DC-SIGN immunoconjugate of any one of Embodiments 188 to 253 or Embodiments 267 to 271, wherein: R^{3a} is —OH, F or —NH₂.

### Embodiment 275

The DC-SIGN immunoconjugate of any one of Embodiments 188 to 253 or Embodiments 267 to 271, wherein: R^{3a} is —OH or F.

### Embodiment 276

The DC-SIGN immunoconjugate of any one of Embodiments 188 to 253 or Embodiments 267 to 271, wherein: R⁵ is —OH, F or —NH₂.

### Embodiment 277

The DC-SIGN immunoconjugate of any one of Embodiments 188 to 253 or Embodiments 267 to 271, wherein: R⁵ is —OH or F.

### Embodiment 278

The DC-SIGN immunoconjugate of any one of Embodiments 188 to 253 or Embodiments 267 to 271, wherein: R^{5a} is —OH, F or —NH₂.

### Embodiment 279

The DC-SIGN immunoconjugate of any one of Embodiments 188 to 253 or Embodiments 267 to 271, wherein: R^{5a} is —OH or F.

### Embodiment 280

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is —OH, and
R^{3a} is F.

### Embodiment 281

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is F, and
R^{3a} is —OH.

### Embodiment 282

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is F, and
R^{3a} is F.

### Embodiment 283

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is —OH, and
R^{3a} is —OH.

### Embodiment 284

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R^{3a} is —OH, and
R⁵ is F.

### Embodiment 285

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R^{3a} is F, and
R⁵ is —OH.

### Embodiment 286

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R^{3a} is F, and
R⁵ is F.

### Embodiment 287

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R^{3a} is —OH, and
R⁵ is —OH.

### Embodiment 288

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is —OH, and
R^{5a} is F.

### Embodiment 289

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is F, and
R^{5a} is —OH.

### Embodiment 290

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is F, and
R^{5a} is F.

### Embodiment 291

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R³ is —OH, and
R^{5a} is —OH.

### Embodiment 292

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R⁵ is —OH, and
R^{5a} is F.

### Embodiment 293

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R⁵ is F, and
R^{5a} is —OH.

### Embodiment 294

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R⁵ is F, and
R^{5a} is F.

### Embodiment 295

The DC-SIGN immunoconjugate of any one of Embodiments 188 to 253 or Embodiments 267 to 271, wherein when present:
R², R^{2a}, R⁴, R^{4a}, R⁶, R^{6a}, R⁷ and R^{7a} are each H;
R⁵ is —OH, and
R^{5a} is —OH.

### Embodiment 296

The DC-SIGN immunoconjugate of any one of Embodiments 188 to 253 or Embodiments 267 to 271, wherein:
R³ is —OH or F;
R^{3a} is —OH or F;
R⁵ is —OH or F;
R^{5a} is —OH or F;
R⁶ is H, and
R^{6a} is H.

### Embodiment 297

The DC-SIGN immunoconjugate of any one of Embodiments 188 to 253 or Embodiments 267 to 271, wherein:
R³ is H, —OH or F;
R^{3a} is H, —OCH₃, —OH or F;
R⁵ is —OH or F;
R⁴, R^{4a}, R⁶, R^{6a}, R⁷, R^{7a} are H, and
R^{6a} is H.

### Embodiment 298

The DC-SIGN immunoconjugate of any one of Embodiments 188 to 298, wherein:
L₁ is —C(═O)O(CH₂)ₘNR¹¹C(═O)(CH₂)ₘ—**; —C(═O)O(CH₂)ₘNR¹¹C(═O)(CH₂)ₘO(CH₂)ₘ—**; —C(═O)O(CH₂)ₘNR¹¹C(═O)X₁X₂C(═O)(CH₂)ₘ—**; —C(═O)OC(R¹²)₂(CH₂)ₘNR¹¹C(═O)X₁X₂C(═O)(CH₂)ₘ—**; —C(═O)O(CH₂)ₘNR⁸C(═O)X₁X₂C(═O)(CH₂)ₘO(CH₂)ₘ—**; —C(═O)O(CH₂)ₘNR¹¹C(═O)X₁X₂C(═O)X₂C(═CH₂)O(CH₂)ₘC(═O)—**; —C(═O)O(CH₂)ₘNR¹¹C(═O)X₄C(═O)NR¹¹ (CH₂)ₘNR¹¹C(═O)(CH₂)ₘO(CH₂)ₘ—**; —C(═O)O(CH₂)ₘNR¹¹C(═O)X₅C(═O)(CH₂)ₘNR¹¹C(═O)(CH₂)ₘ—**; —C(═O)O(CH₂)ₘX₆C(═O)X₁X₂C(═O)((CH₂)ₘO)ₙ(CH₂)ₘ**; —(CH₂)ₘNR¹¹C(═O)X₁X₂C(═O)(CH₂)ₘ—**; —(CH₂)ₘ(CHOH)(CH₂)ₘNR¹¹C(═O)X₁X₂C(═O)(CH₂)ₘ**; —C(═O)X₆C(═O)(CH₂)ₘNR¹¹C(═O)X₁X₂C(═O)(CH₂)ₘ—**; —C(═O)X₄C(═O)NR(CH₂)ₘNR¹¹C(═O)(CH₂)ₘO(CH₂)ₘ—** C(═O)(CH₂)ₘNR¹¹C(═O)X₁X₂C(═O)(CH₂)ₘ—**, —C(═O)O(CH₂)ₘX₆C(═O)X₁X₂C(═O)(CH₂)ₘ—**, or —C(═O)(CH₂)ₘNR¹¹C(═O)((CH₂)ₘO)ₙ(CH₂)ₘ—**;
where the ** of L, indicates the point of attachment to R¹⁵ and
where R¹¹, R¹², X₁, X₂, m and n are s defined in Embodiment 205.

### Embodiment 299

A DC-SIGN immunoconjugate selected from:

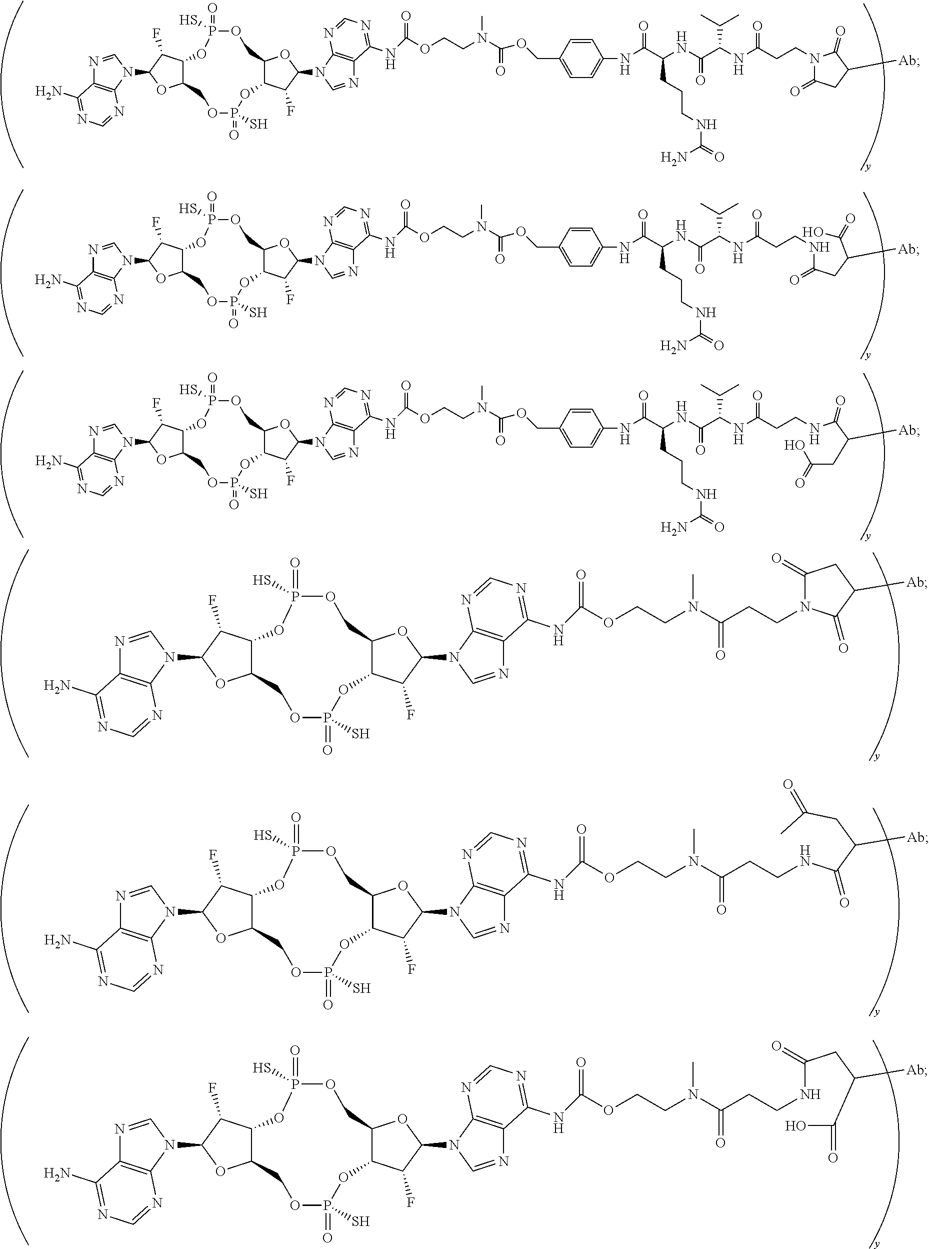

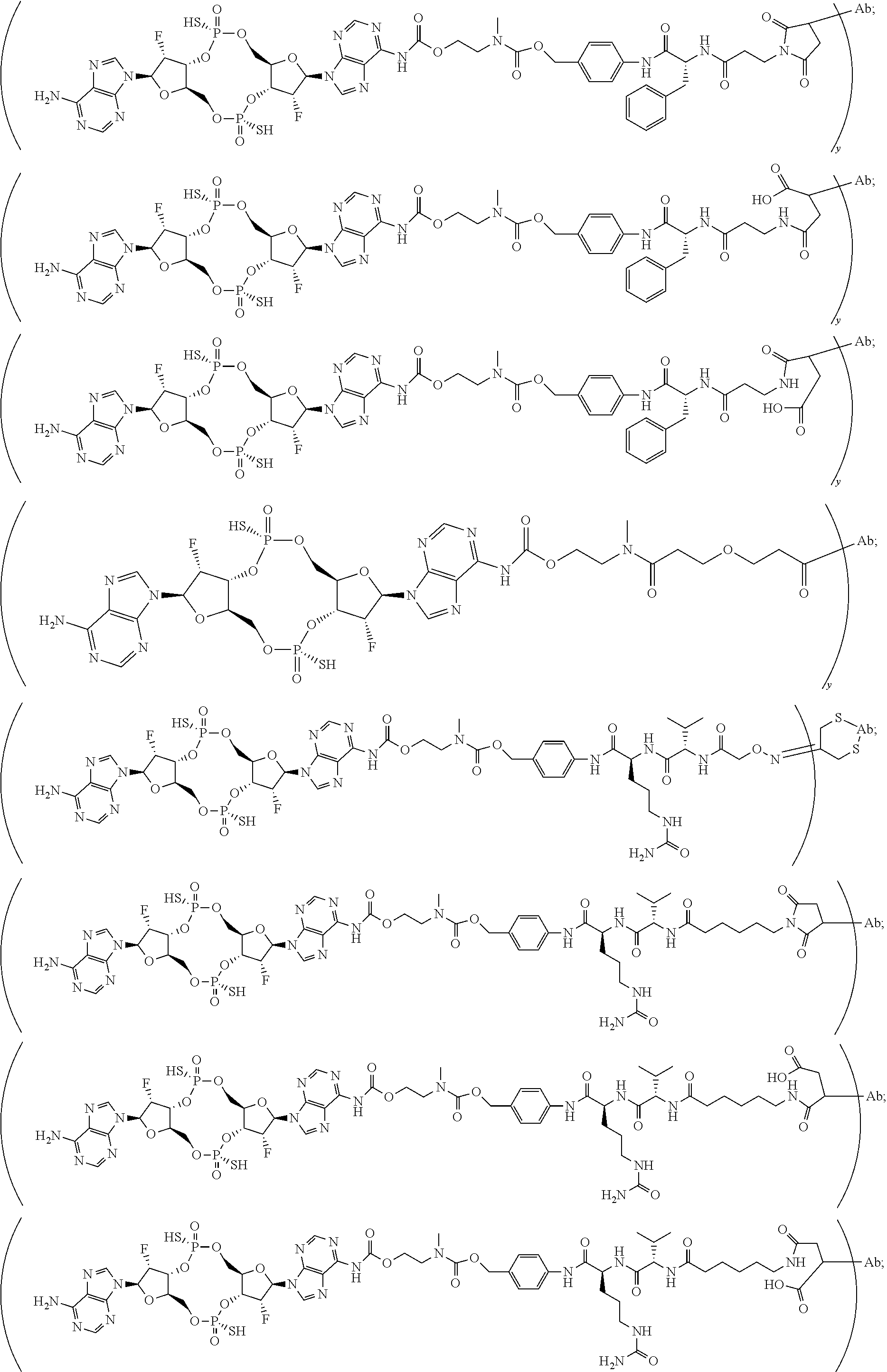

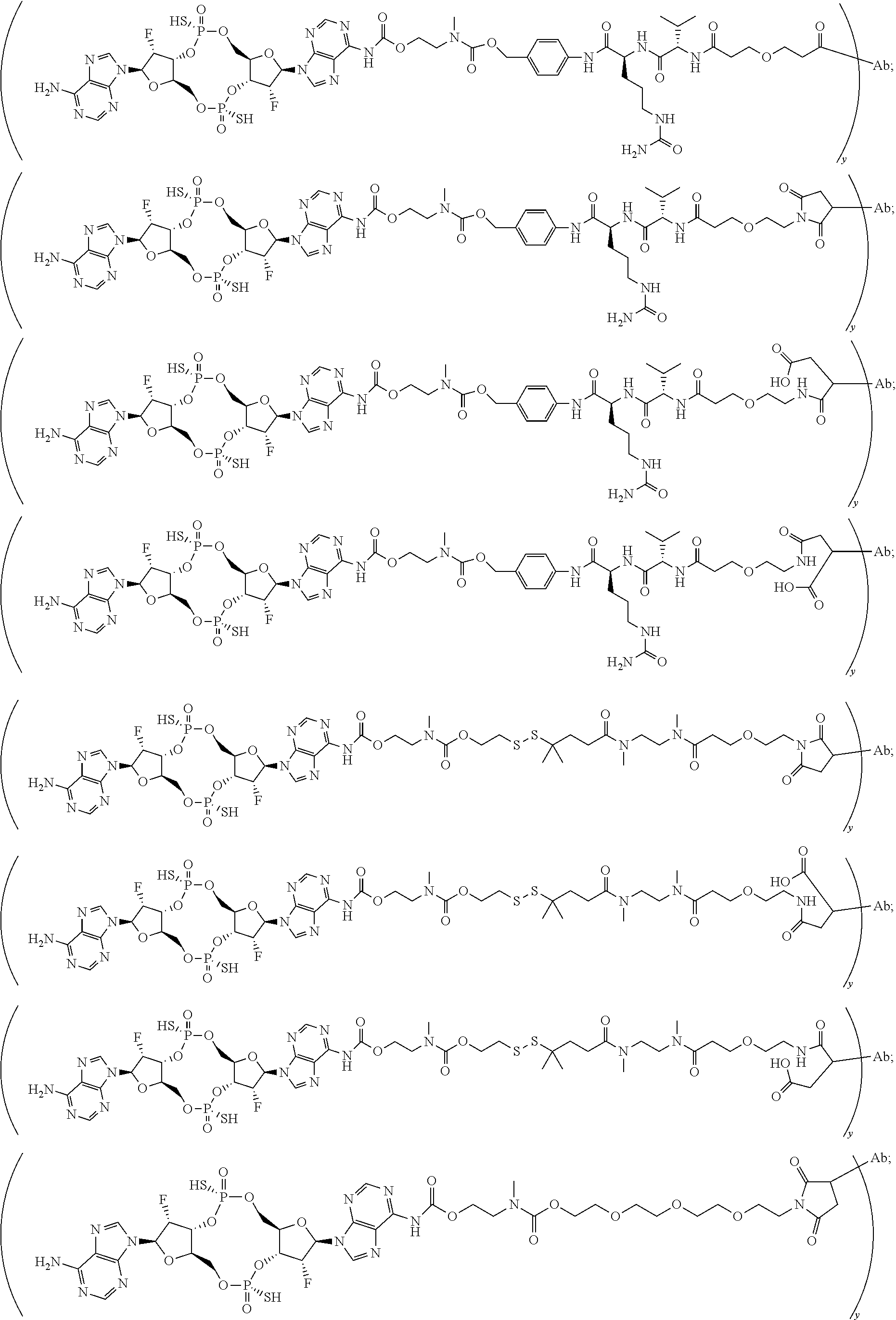

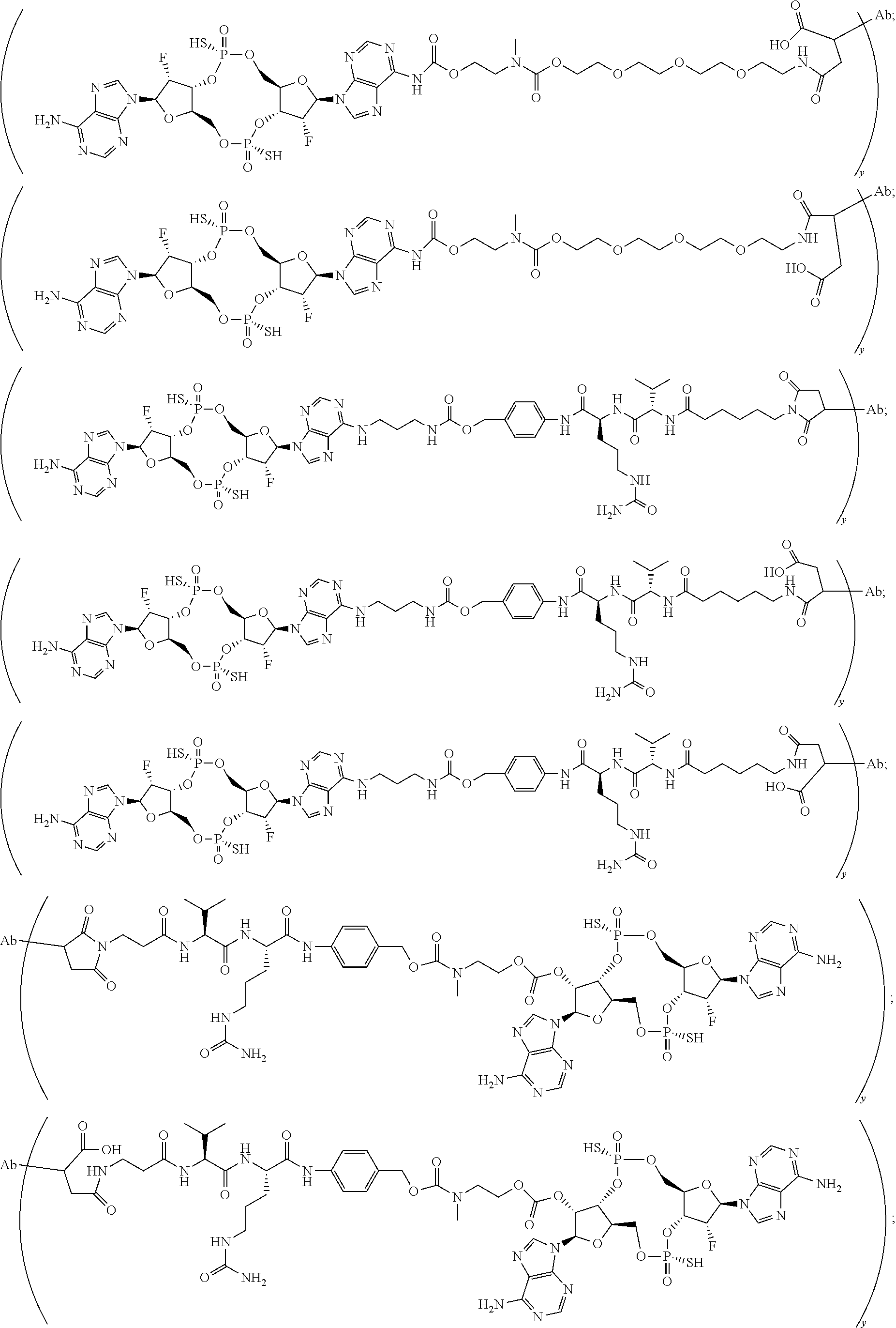

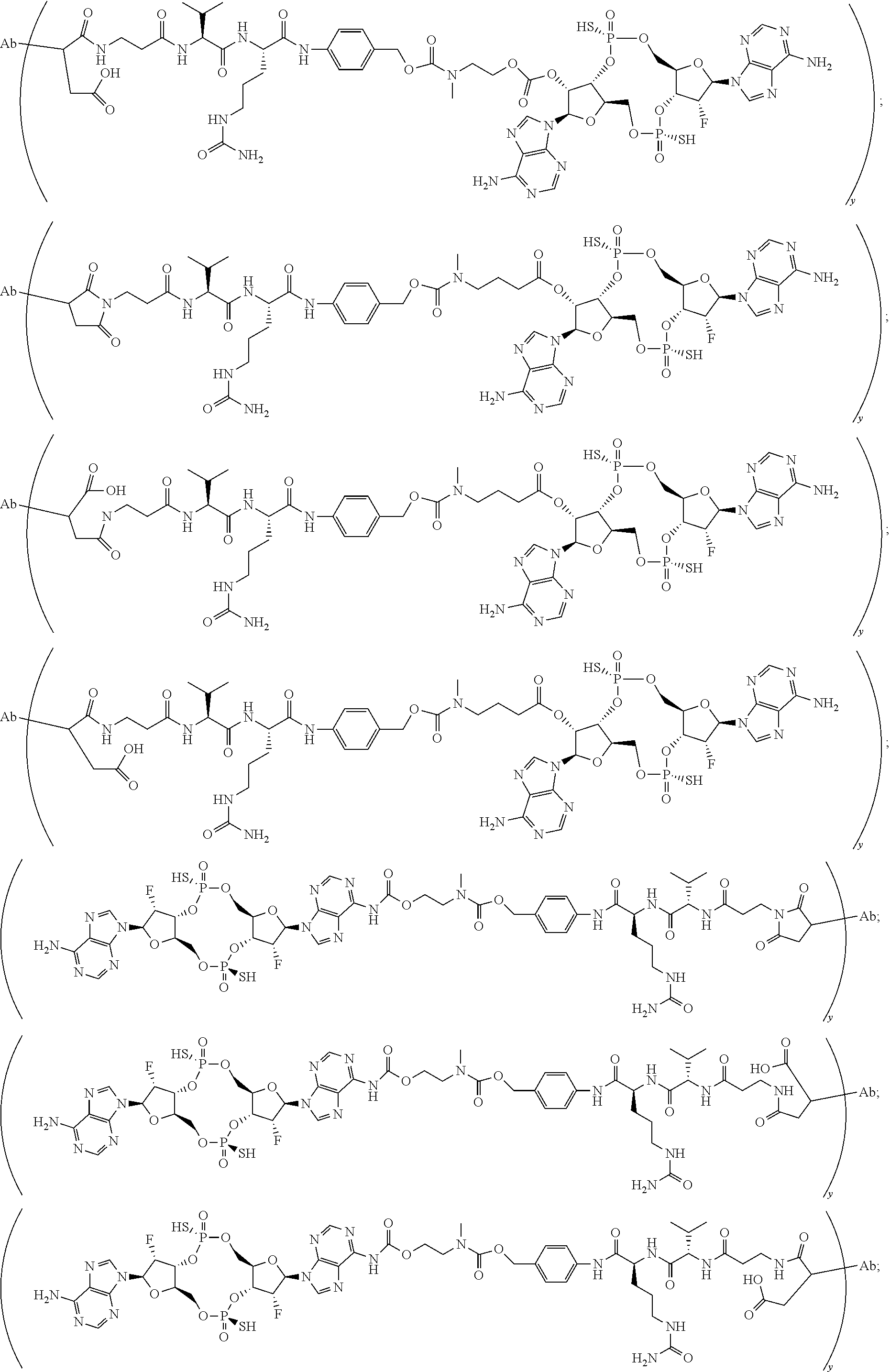

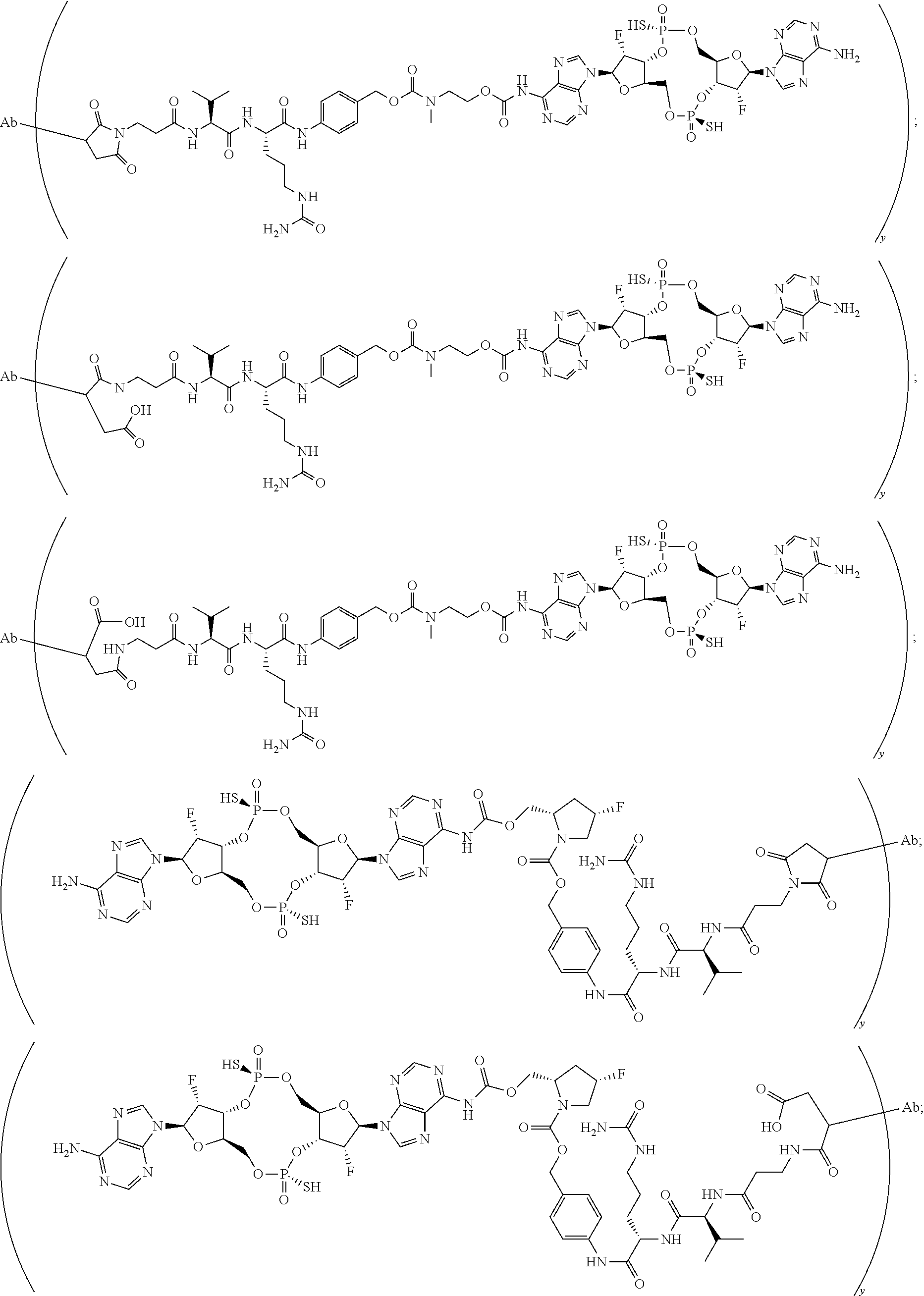

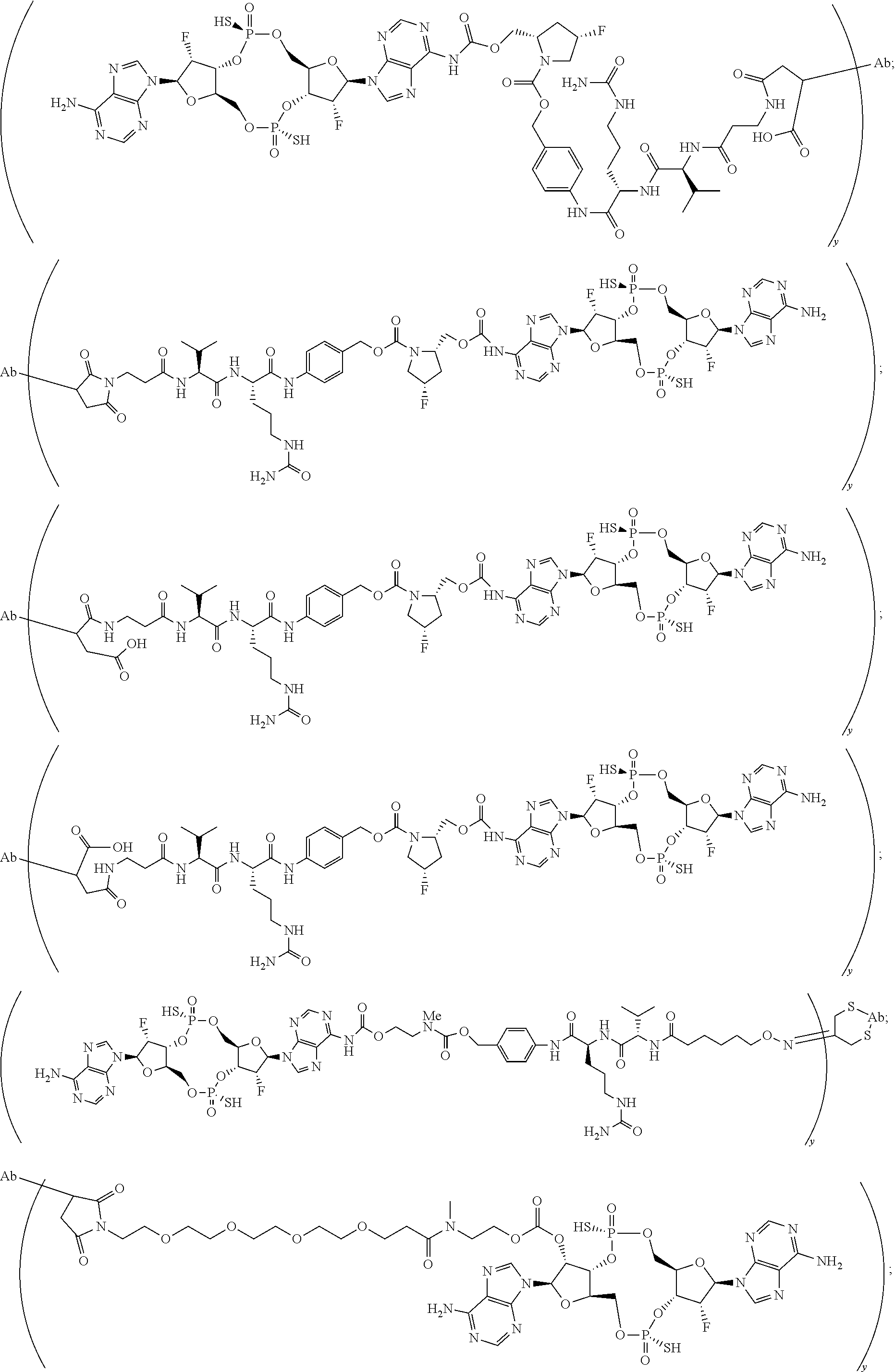

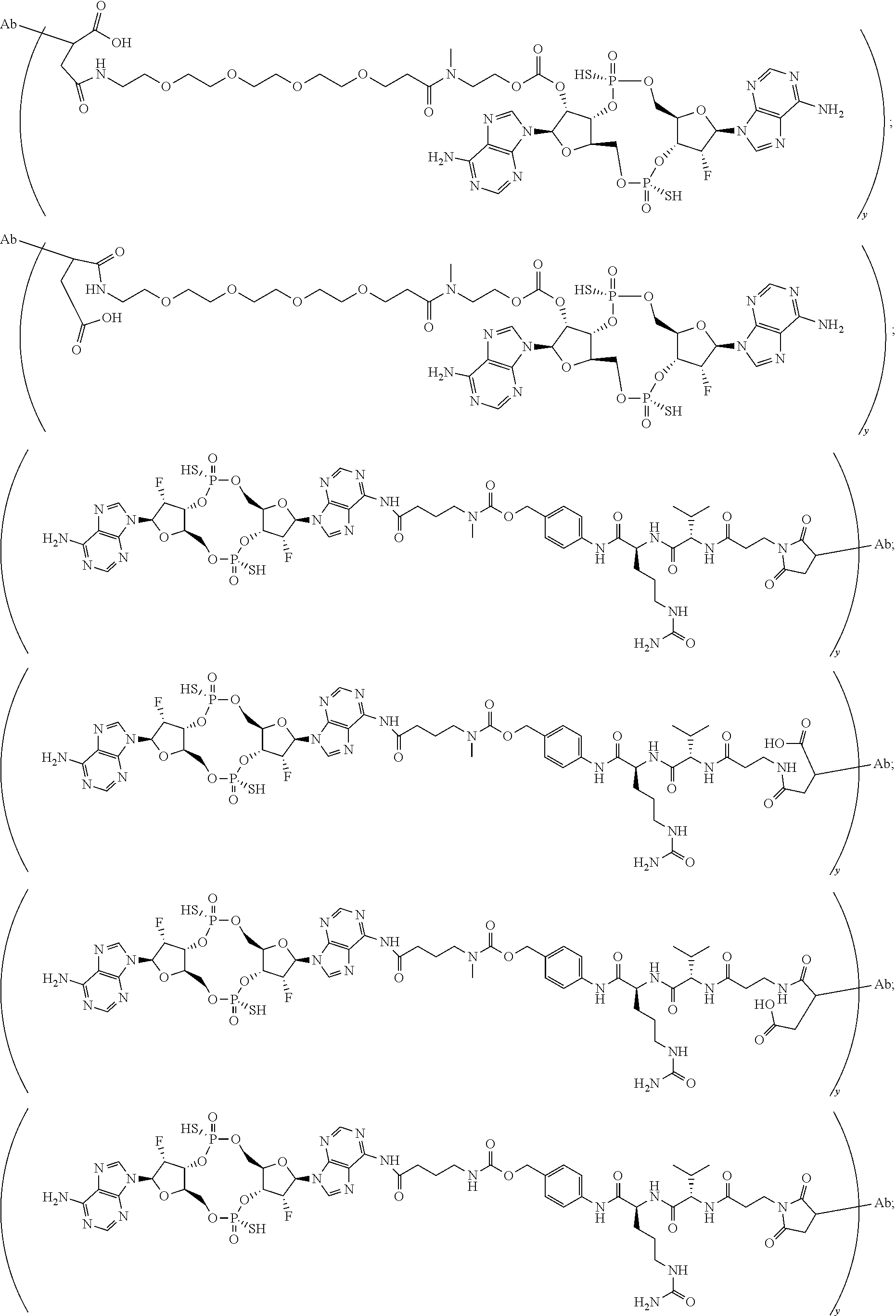

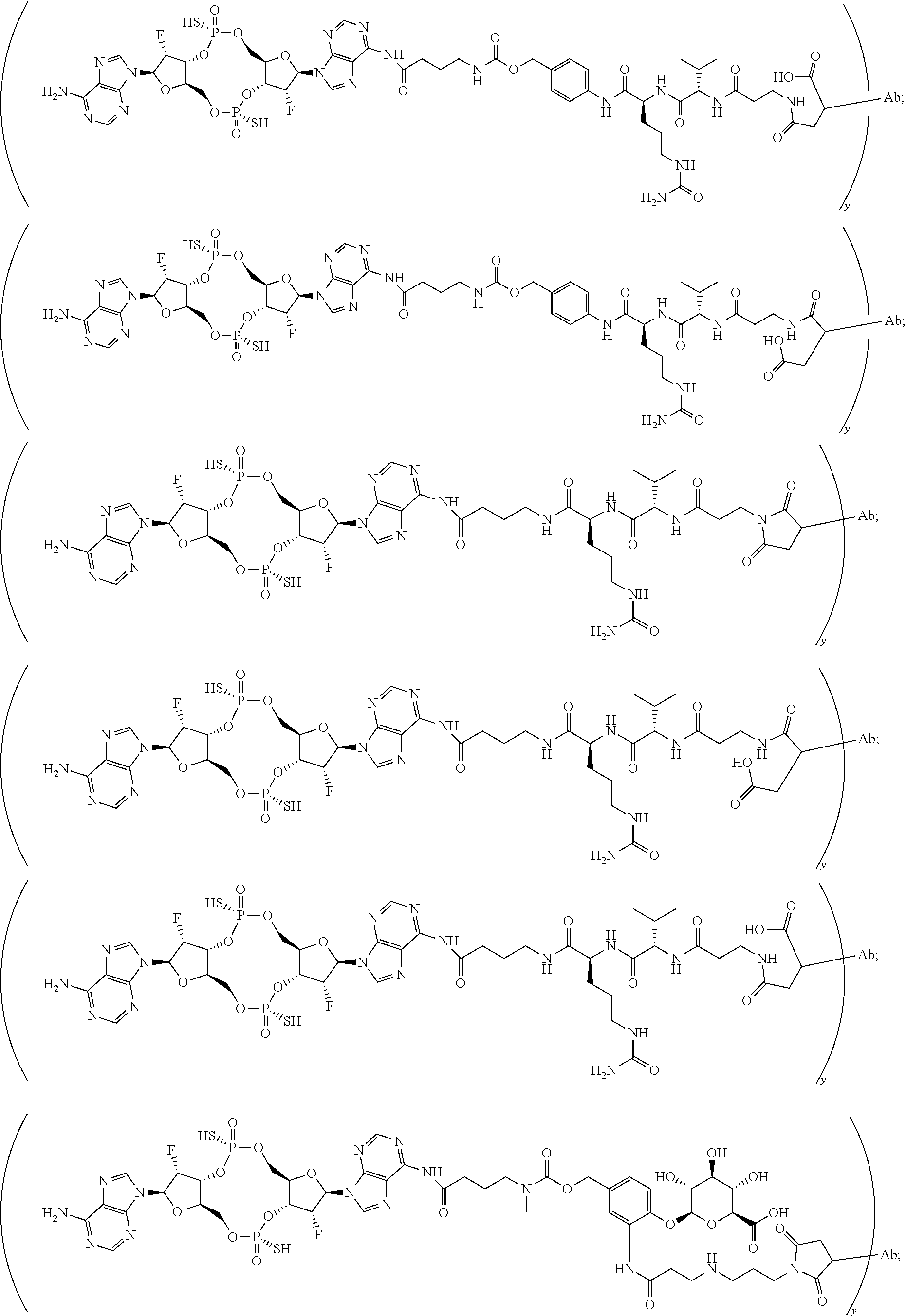

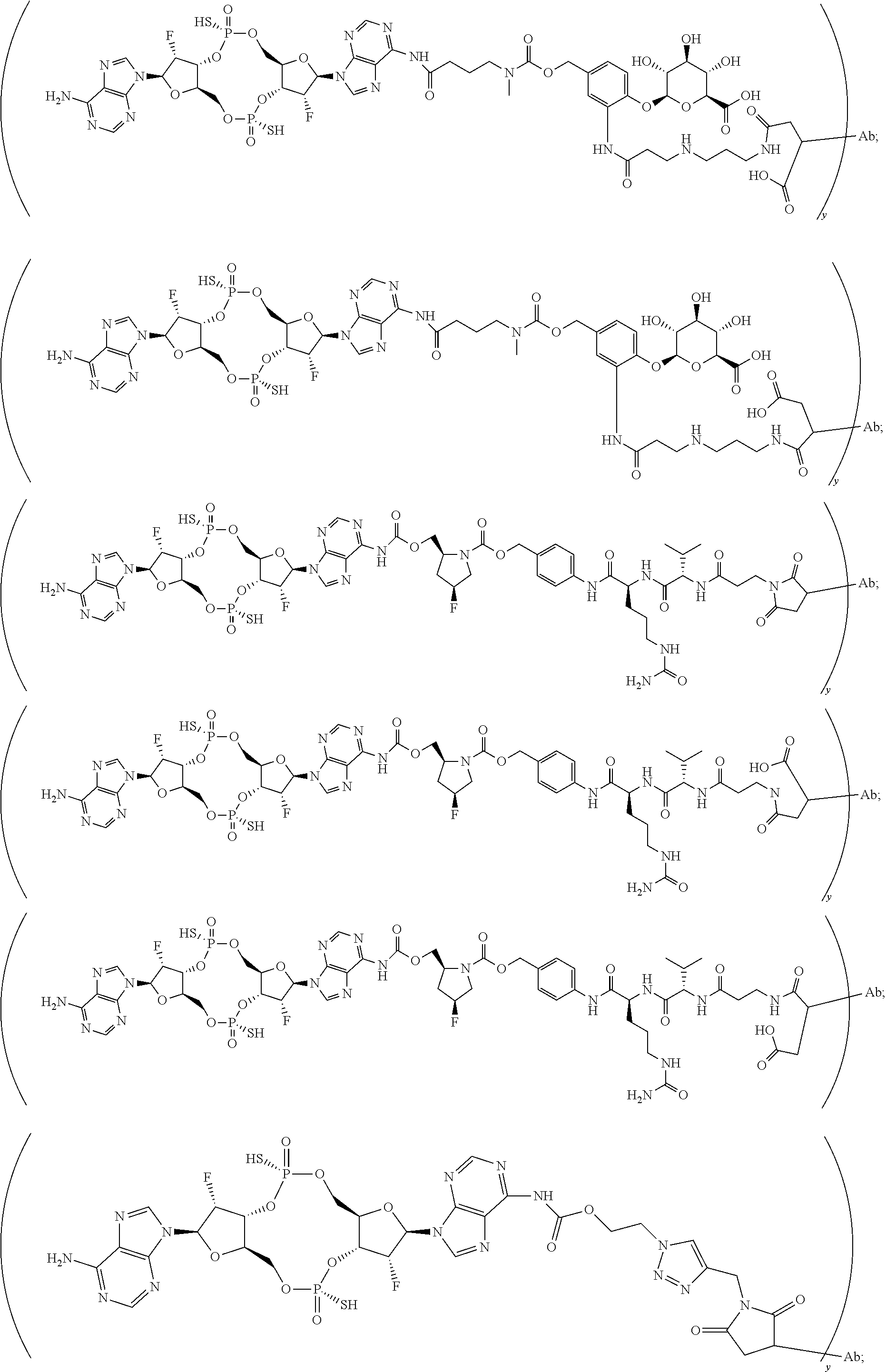

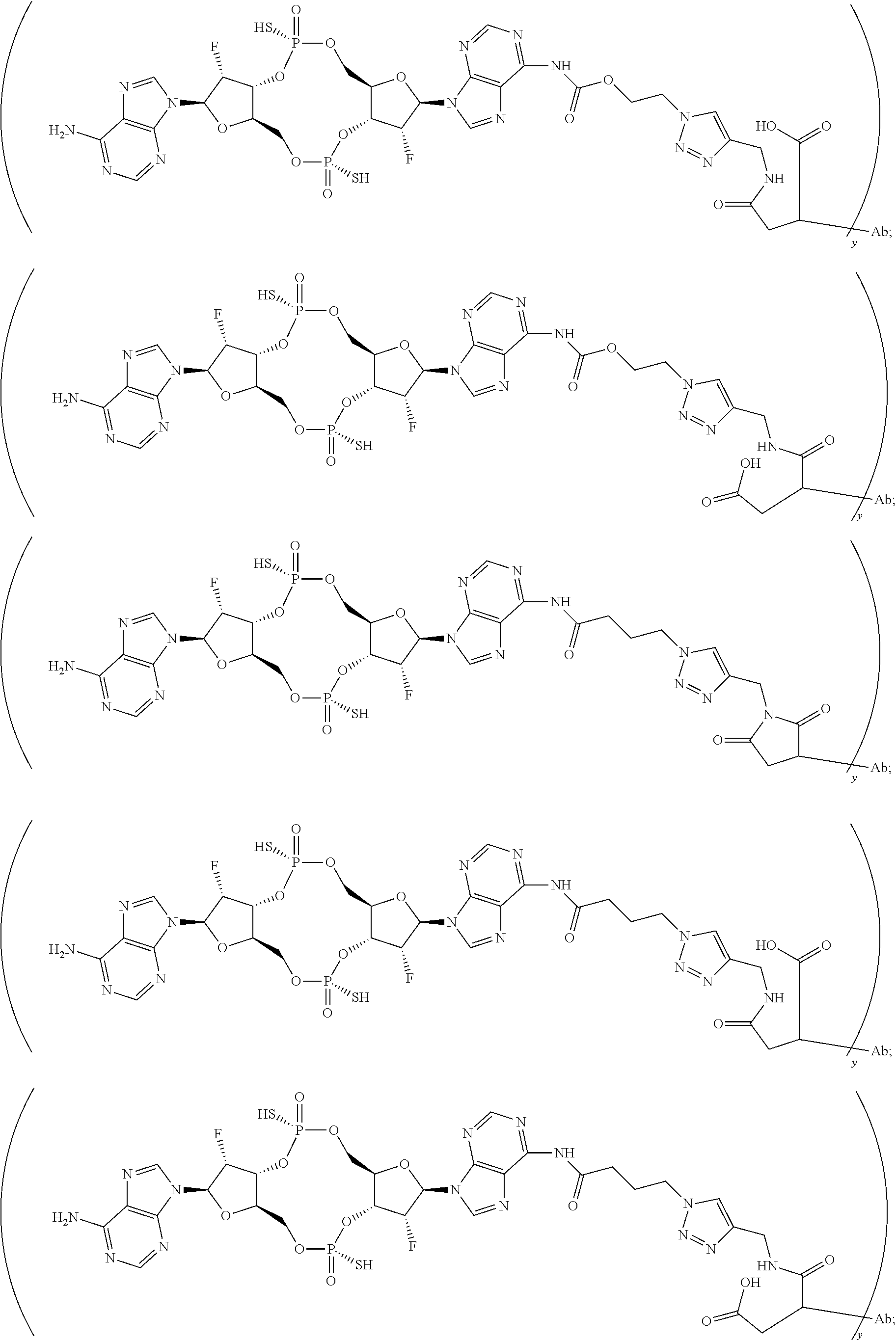

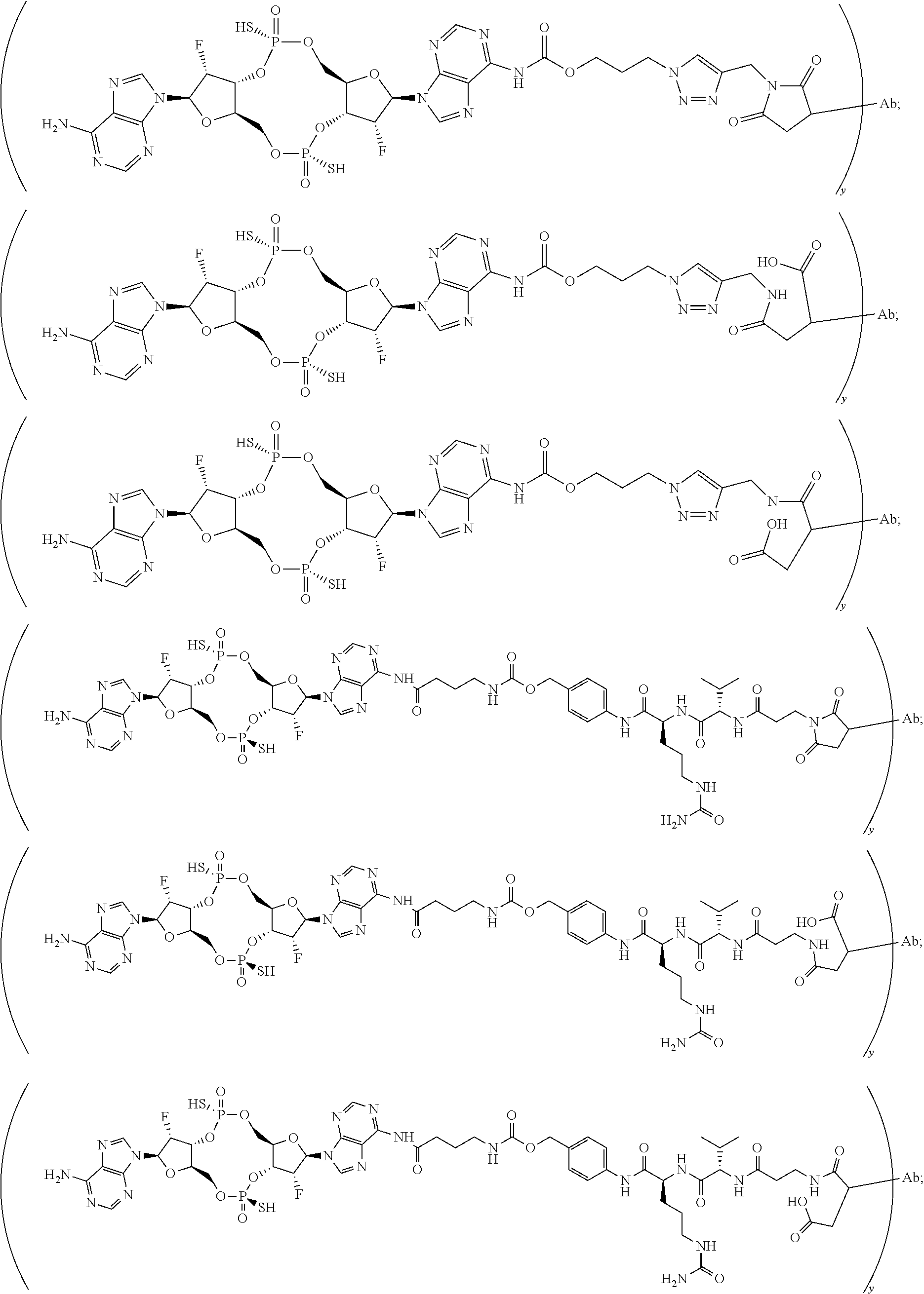

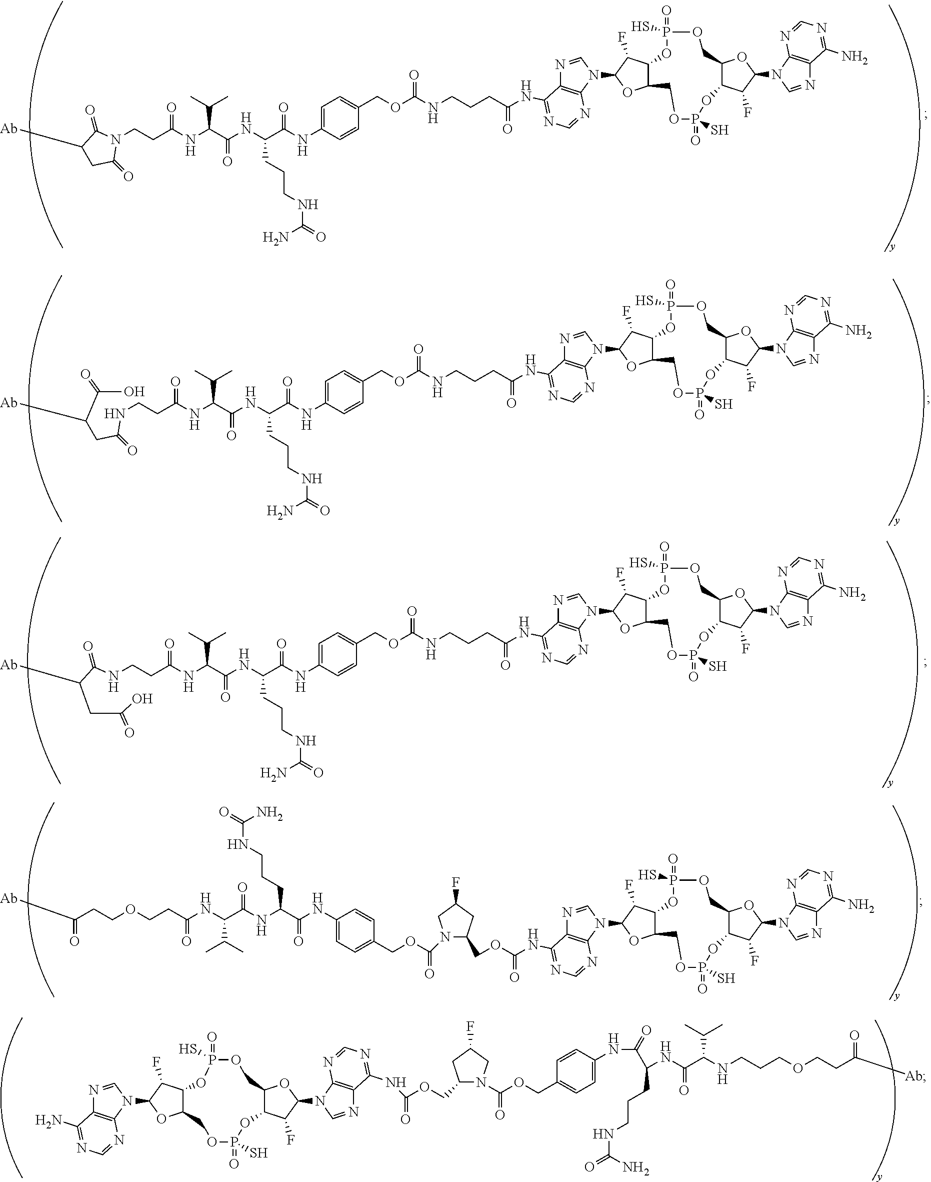

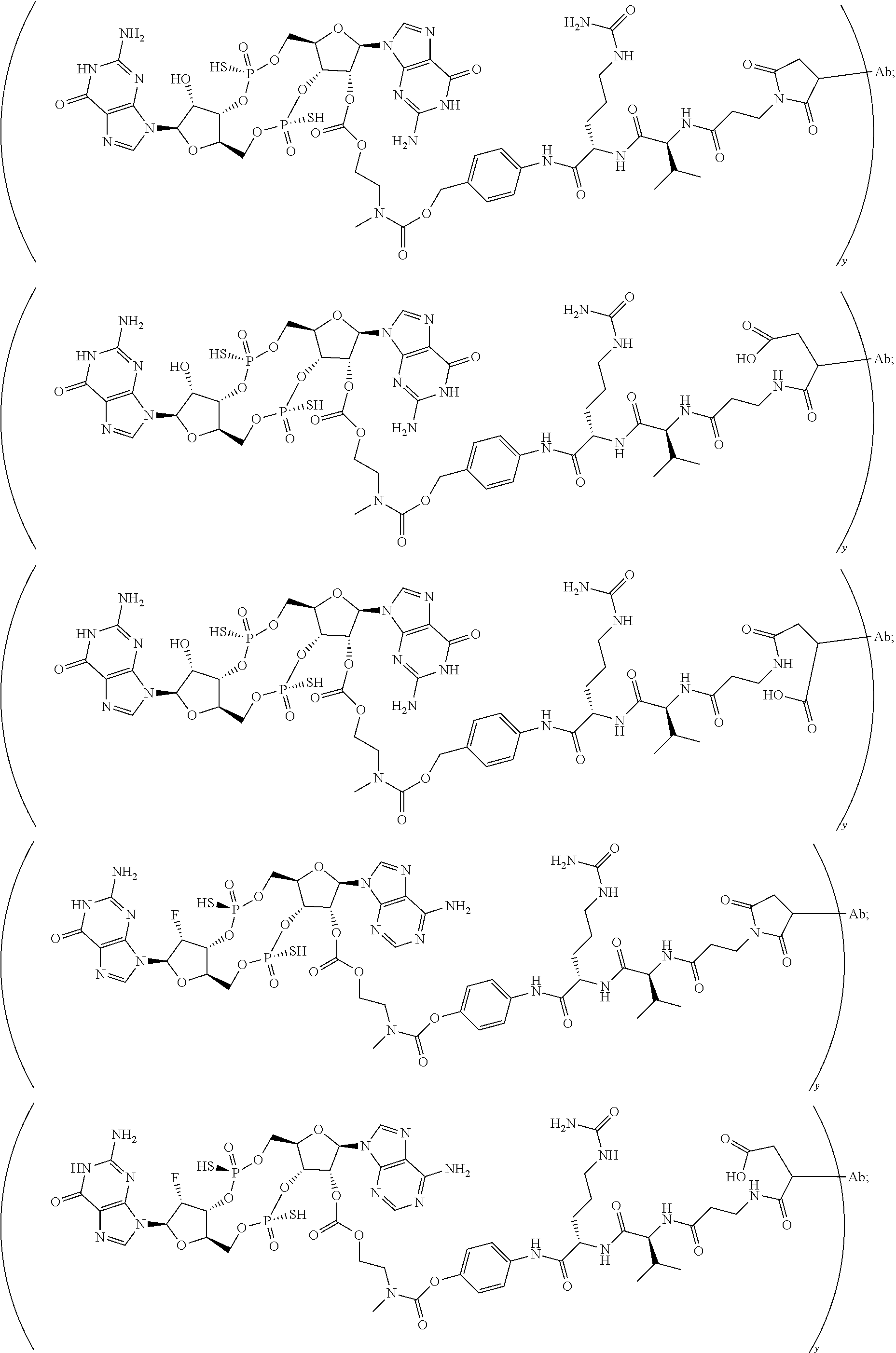

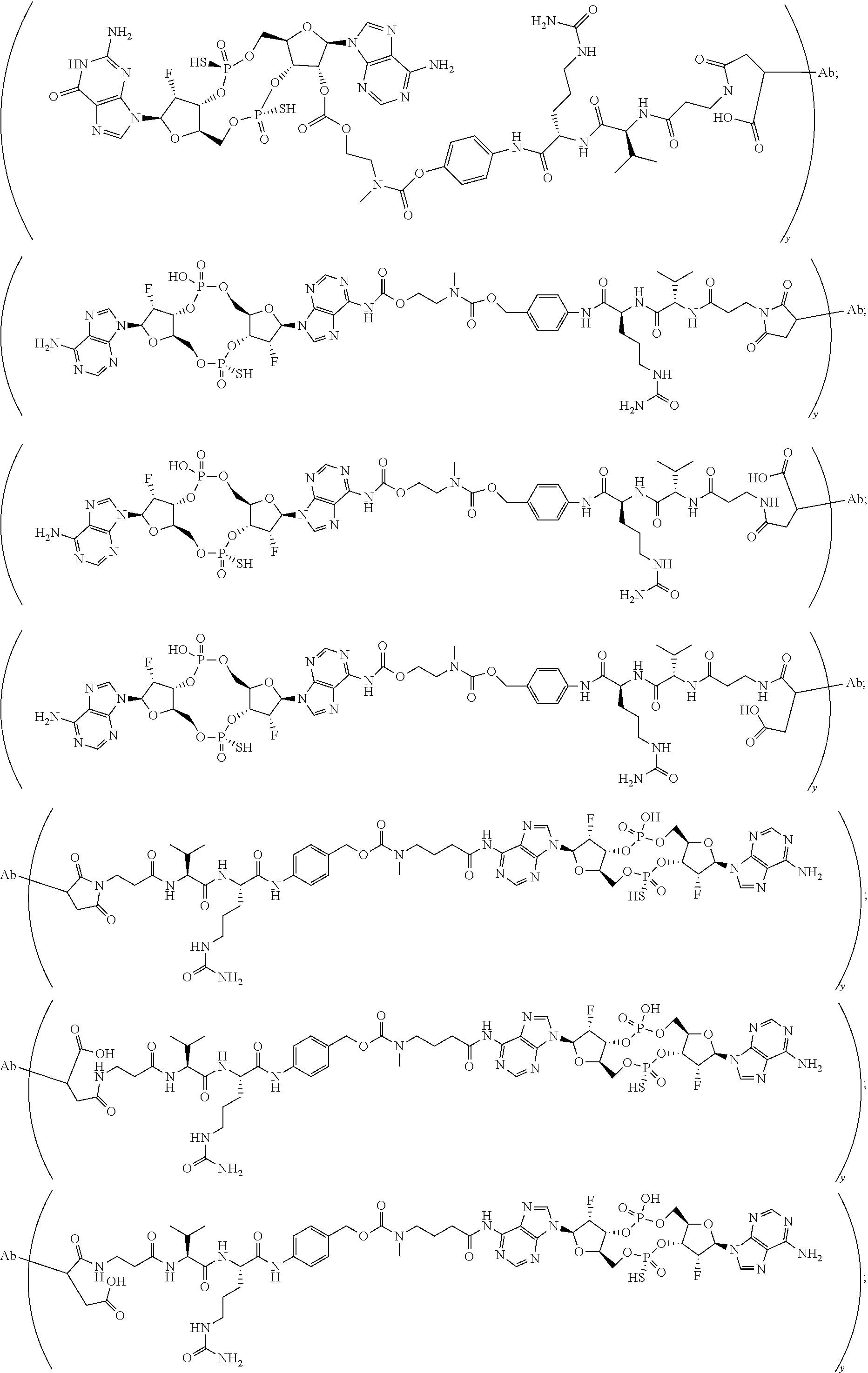

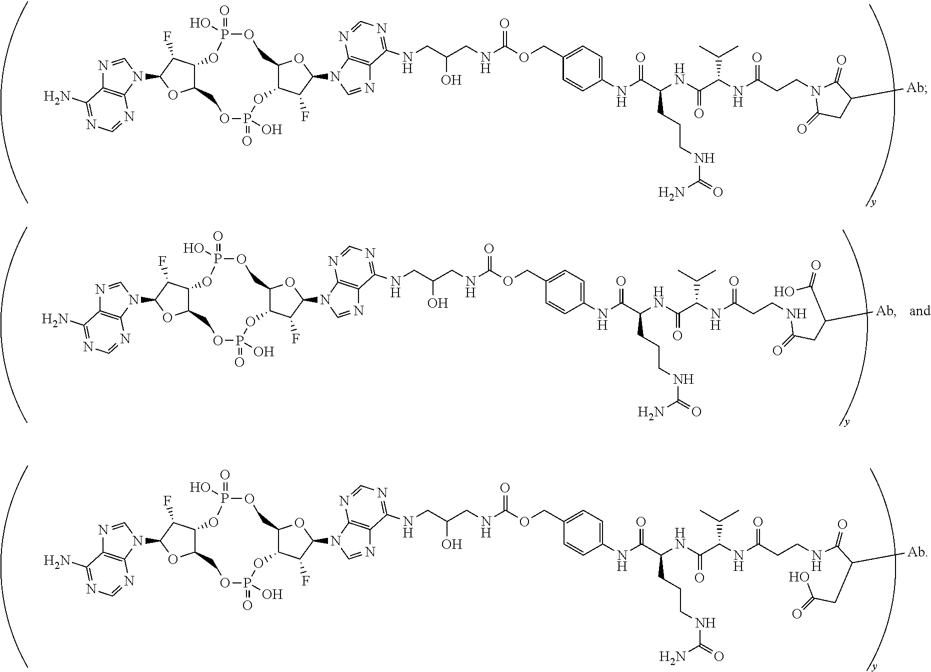

### Embodiment 300

A DC-SIGN immunoconjugate selected from:

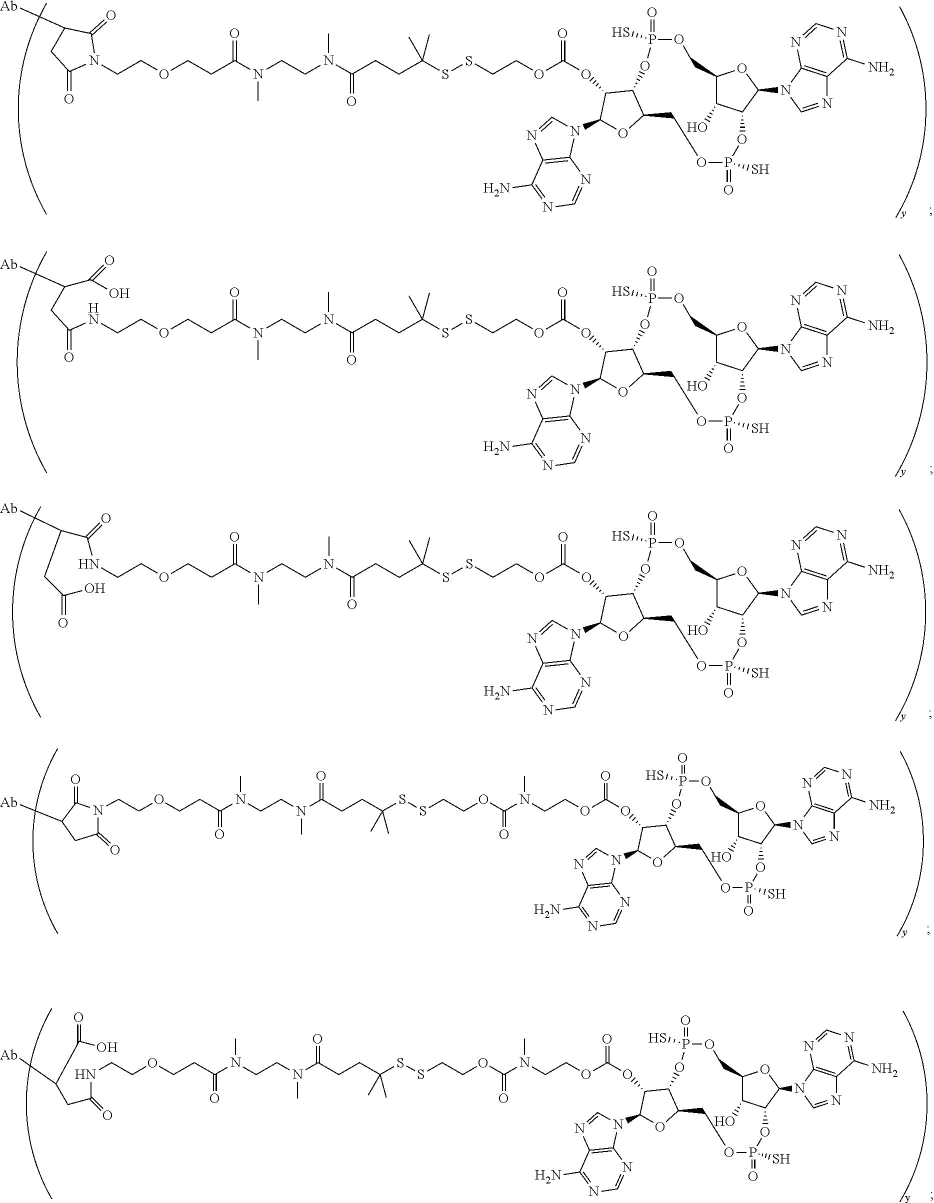

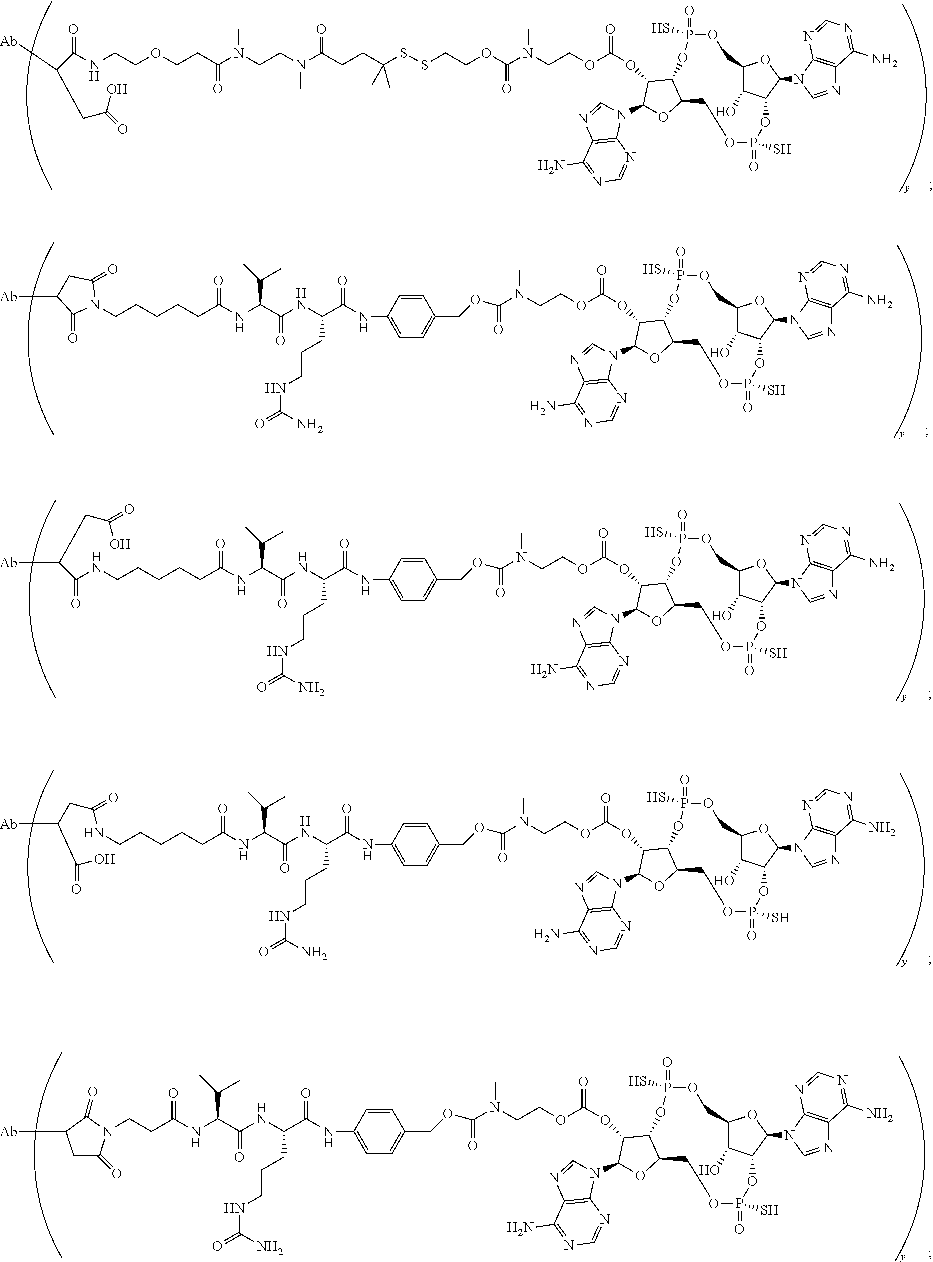

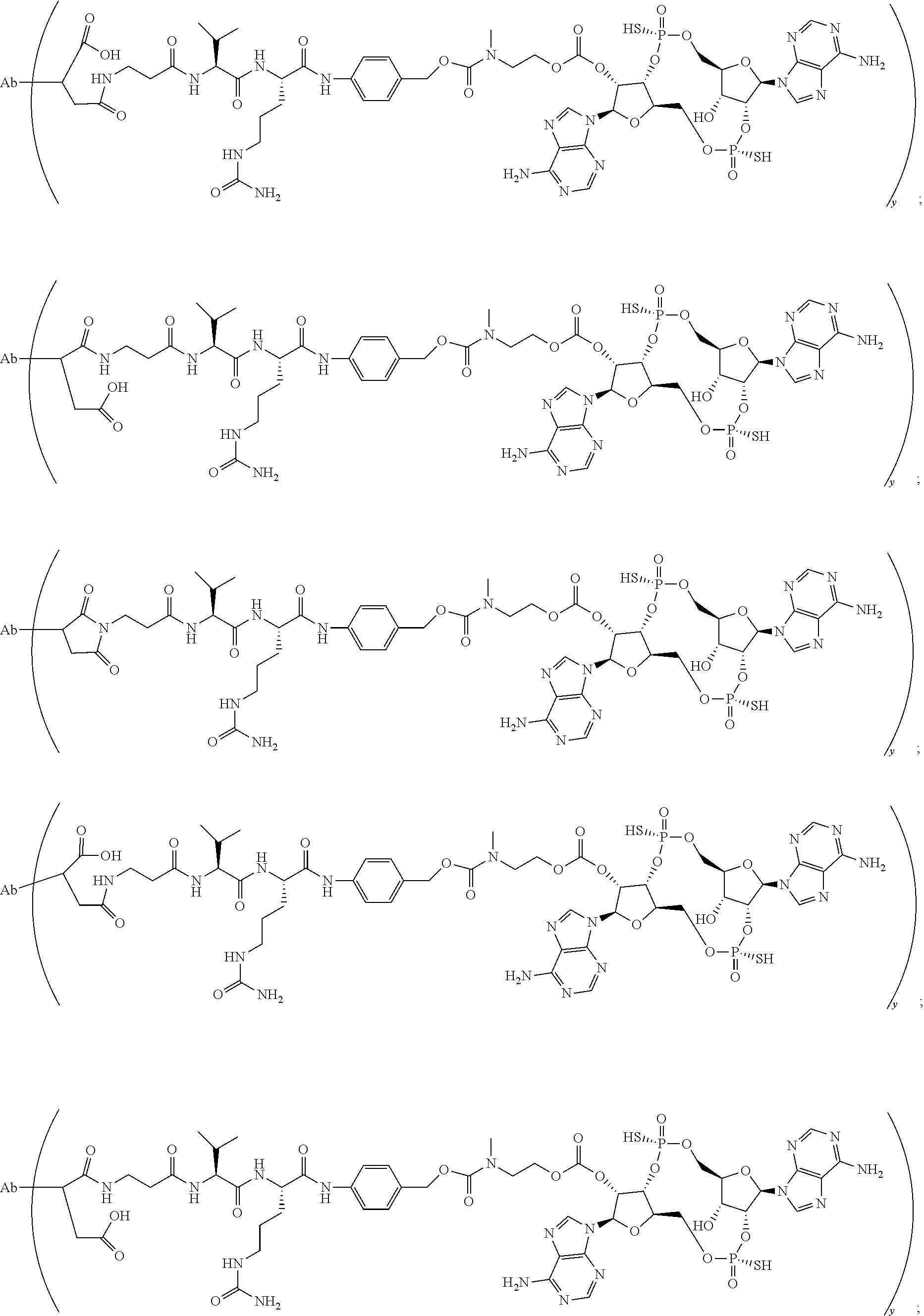

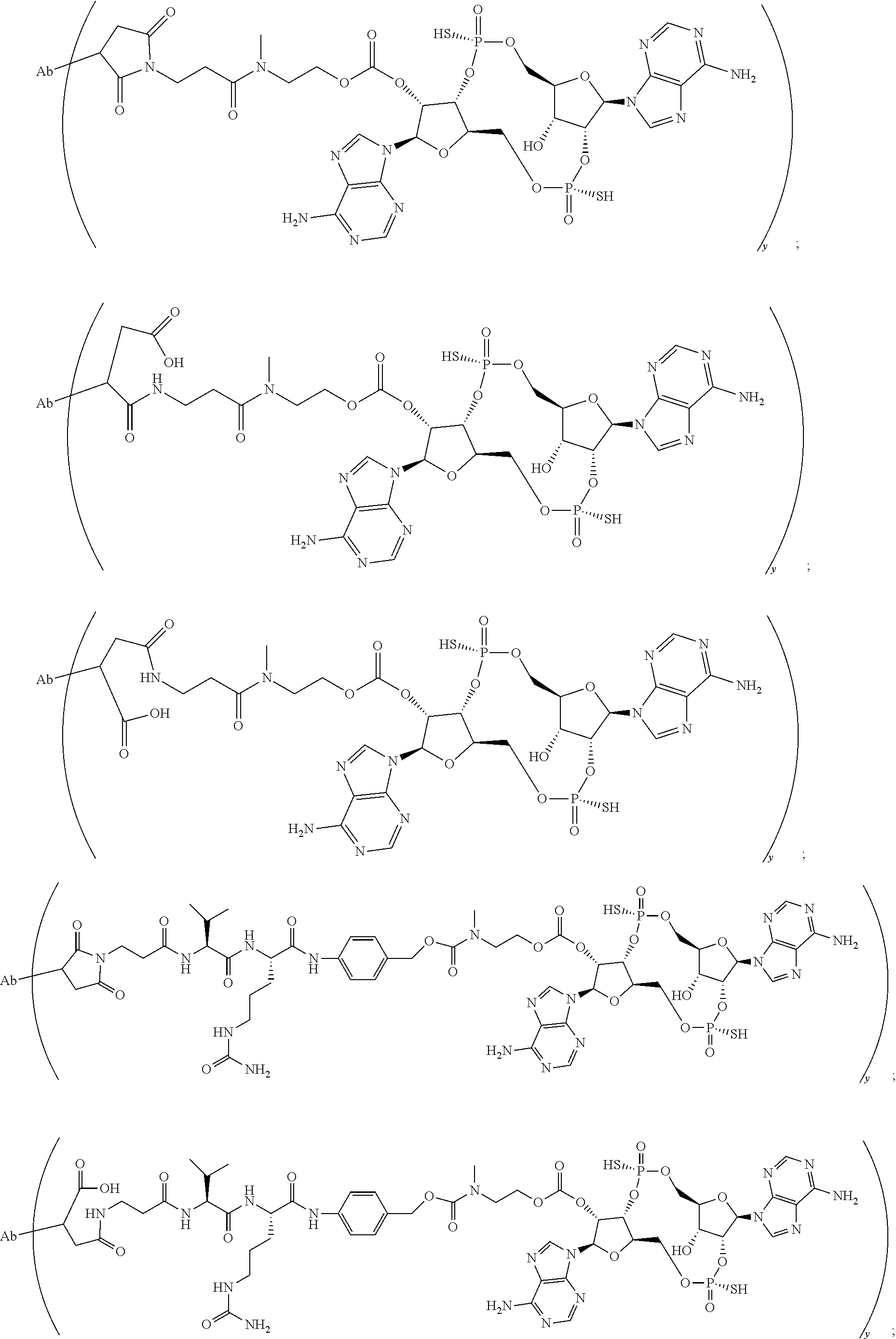

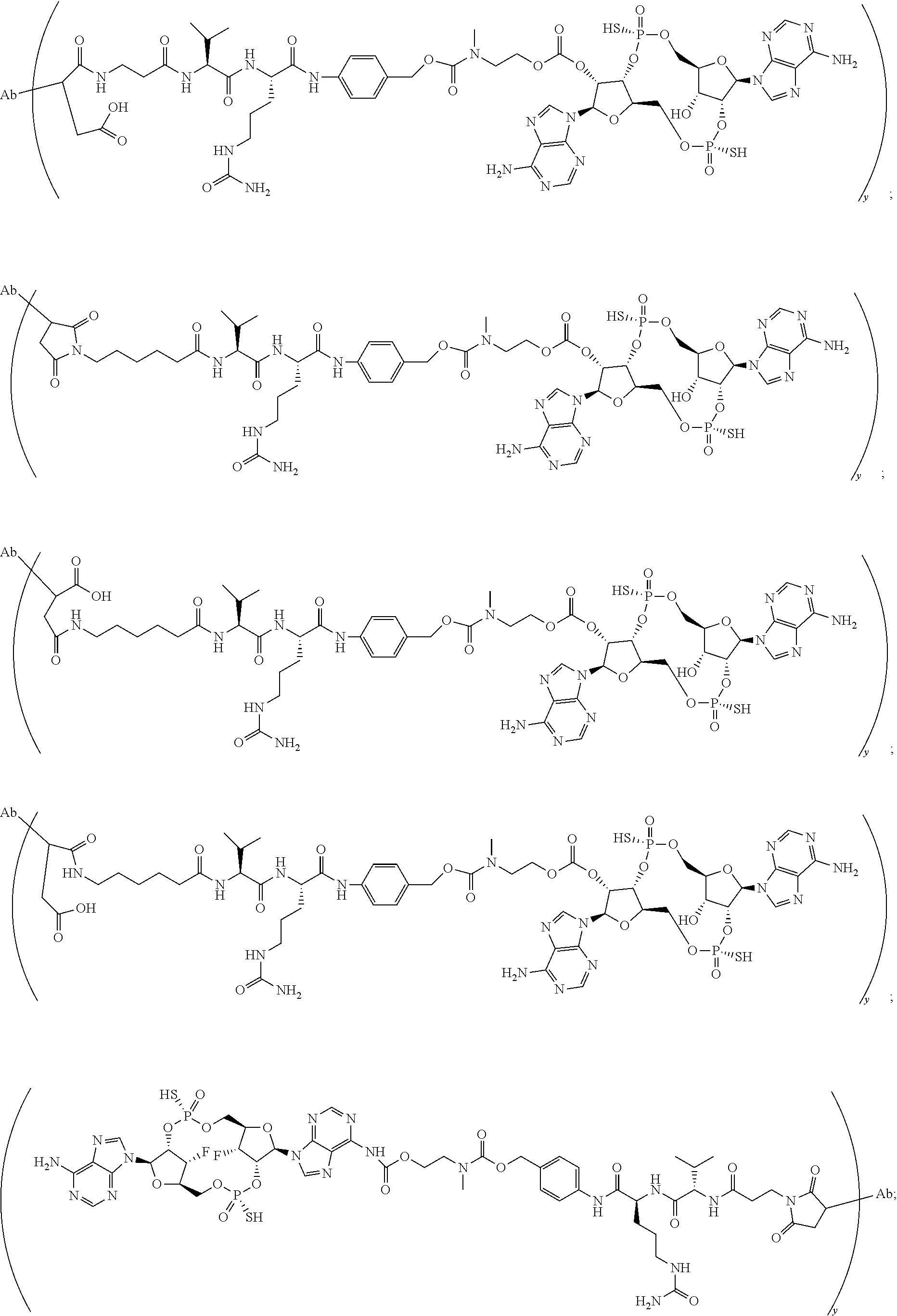

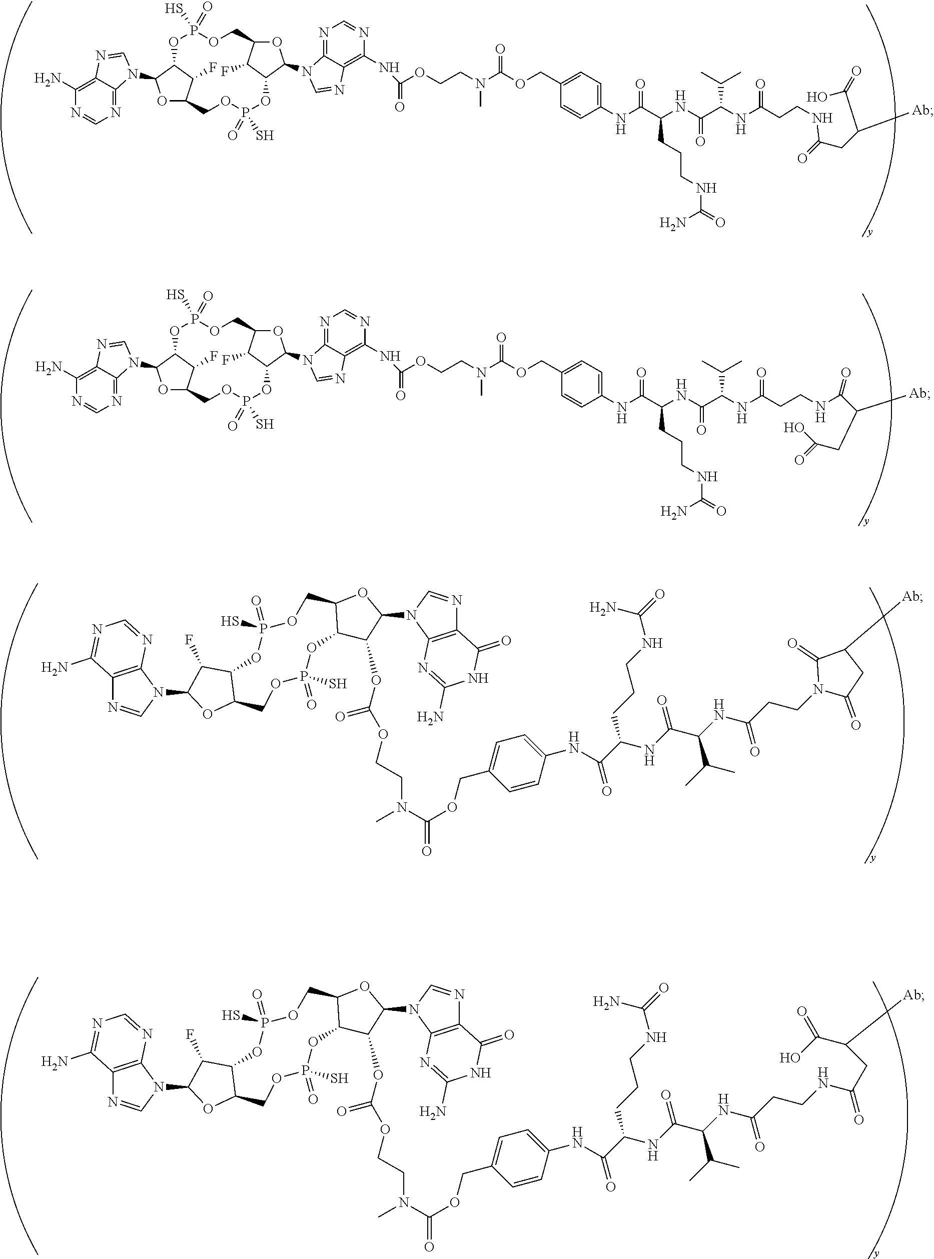

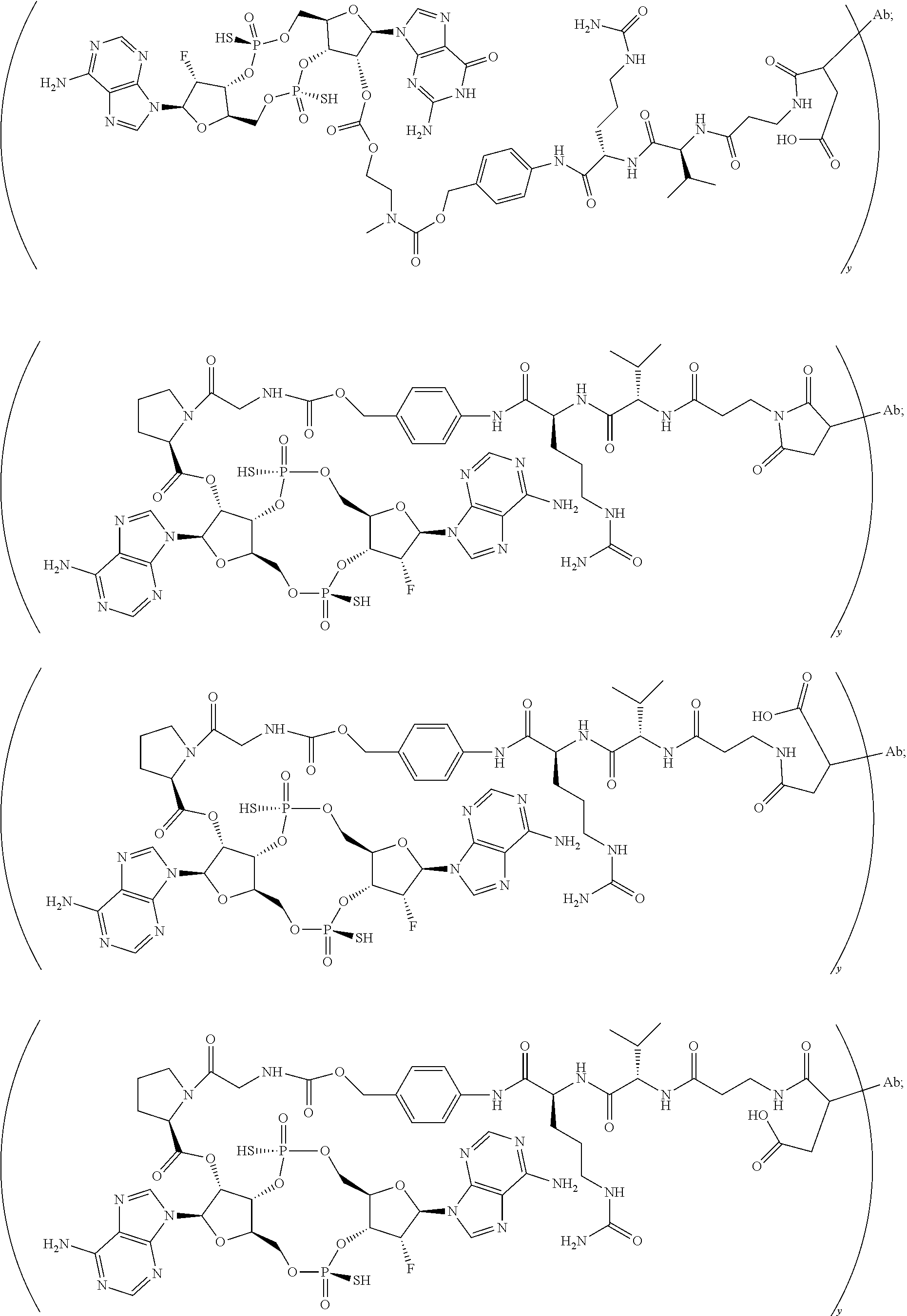

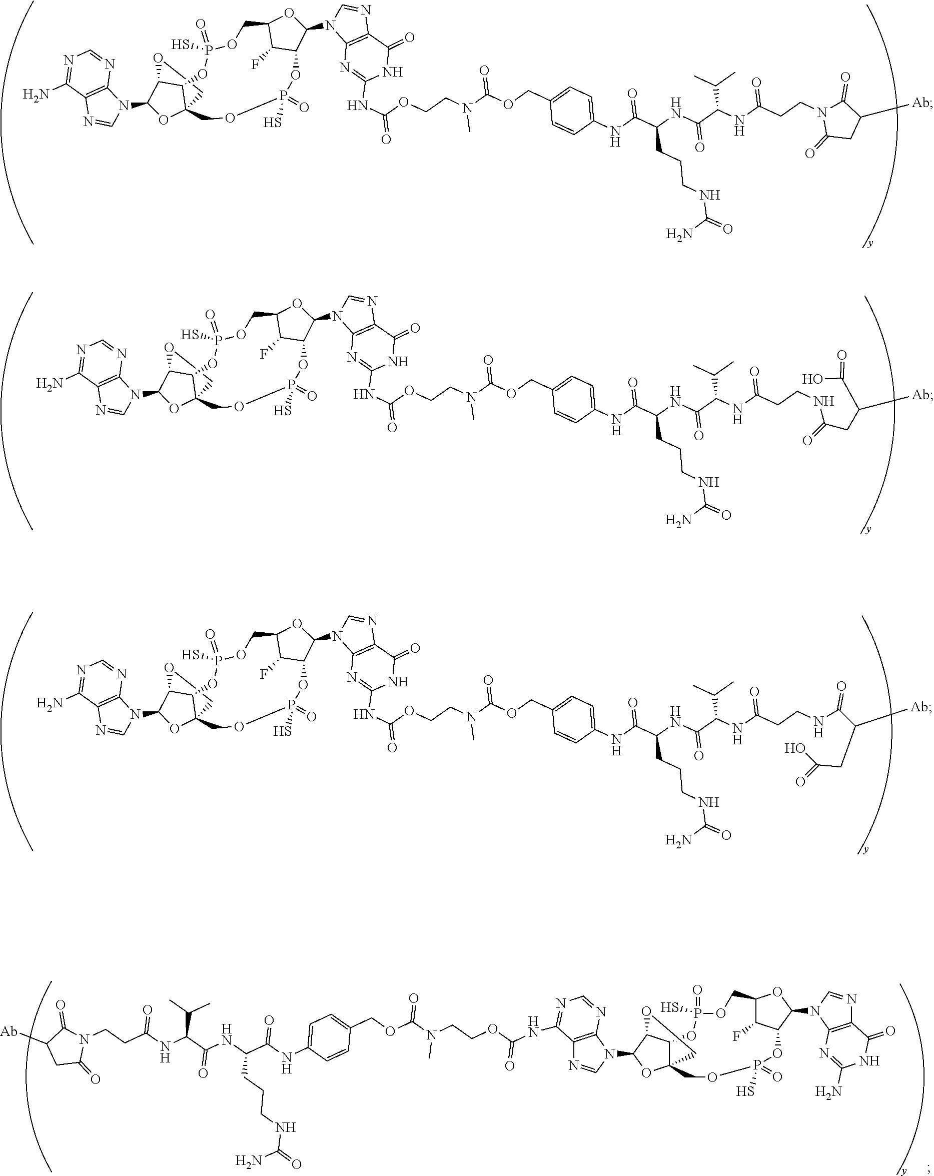

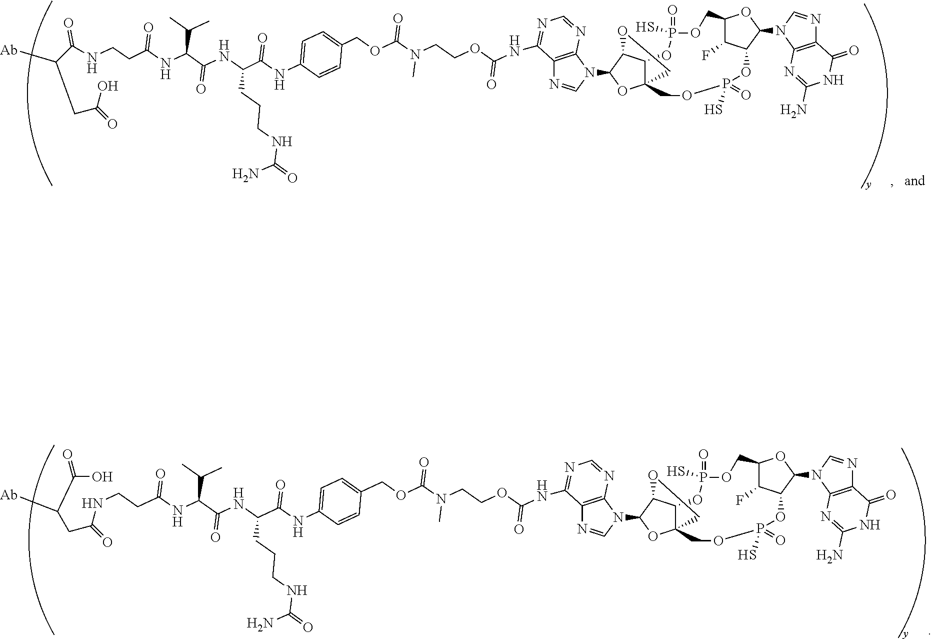

Provided are also protocols for some aspects of analytical methodology for evaluating DC-SIGN antibody conjugates of the invention. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on otherxample properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation over time, adherence to surfaces, hepatotoxicity, clearance rates, and pharmacokinetic exposure. See Damle, N. K., Nat Biotechnol. 2008; 26(8):884-885; Singh, S. K., Pharm Res. 2015; 32(11):3541-71. When measured by hydrophobic interaction chromatography, higher hydrophobicity index scores (i.e. elution from HIC column faster) reflect lower hydrophobicity of the conjugates. As shown in Examples below, a majority of the tested antibody conjugates showed a hydrophobicity index of greater than 0.8. In some embodiments, provided are antibody conjugates having a hydrophobicity index of 0.8 or greater, as determined by hydrophobic interaction chromatography.

### Anti-DC-SIGN Antibody

In some embodiments, antibody conjugates provided herein include an antibody or antibody fragment thereof (e.g., antigen binding fragment) that specifically binds to human DC-SIGN (anti-DC-SIGN antibody). DC-SIGN overexpression is observed in macrophages and dendritic cells in tumor microenvrionment as well as in lymphoid and peripheral tissues. Antibody conjugates comprising an anti-DC-SIGN antibody can be specifically targeted to macrophages and dendritic cells in tumors and/or lymphoid and peripheral tissues.

In some embodiments, DC-SIGN antibody conjugates provided herein include a monoclonal antibody or antibody fragment thereof that specifically binds to human DC-SIGN, e.g., a human or humanized anti-DC-SIGN monoclonal antibody. In some embodiments, the antibody or antibody fragment thereof that specifically binds to human DC-SIGN can be selected from the anti-DC-SIGN antibodies disclosed herein.

Suitable anti-DC-SIGN monoclonal antibodies include, but are not limited to, the anti-DC-SIGN antibodies described in U.S. Pat. Nos. 7,534,866; 7,786,267; 7,846,744; 8,409,577; 8,779,107; 8,883,160; 8,916,696; PCT Publication Nos: WO2004091543; WO2005027979; WO2006066229; WO2006081576; WO2007046893; WO2008011599; WO2010053561; WO2011031736; WO2012145209; WO2013009841; WO2013024059; WO2013049307; WO2013095966; WO2013142255; WO2013125891; WO2013163689; WO2014064187; WO2014083499; WO2014144960; WO2014176604; WO2014179601; WO2015004473; WO2015023355; WO2015048633; WO2015048641; WO2015054039; WO2015073307; WO2015112626; U.S. Patent Publication No: US2014045242; and Chinese Patent Publication No: CN103739714, the contents of which are herein incorporated by reference in their entireties.

In some embodiments, the anti-DC-SIGN antibody or antibody fragment (e.g., an antigen binding fragment) comprises a VH domain having an amino acid sequence of any VH domain described in Table 8. Other suitable anti-DC-SIGN antibodies or antibody fragments (e.g., antigen binding fragments) can include amino acids that have been mutated, yet have at least 80, 85, 90, 95, 96, 97, 98, or 99 percent identity in the VH domain with the VH regions depicted in the sequences described in Table 8. The present disclosure in certain embodiments also provides antibodies or antibody fragments (e.g., antigen binding fragments) that specifically bind to DC-SIGN, wherein the antibodies or antibody fragments (e.g., antigen binding fragments) comprise a VH CDR having an amino acid sequence of any one of the VH CDRs listed in Table 8. In particular embodiments, the invention provides antibodies or antibody fragments (e.g., antigen binding fragments) that specifically bind to DC-SIGN, comprising (or alternatively, consist of) one, two, three, four, five or more VH CDRs having an amino acid sequence of any of the VH CDRs listed in Table 8.

In some embodiments, the anti-DC-SIGN antibody or antibody fragment (e.g., antigen binding fragments) comprises a VL domain having an amino acid sequence of any VL domain described in Table 8. Other suitable anti-DC-SIGN antibodies or antibody fragments (e.g., antigen binding fragments can include amino acids that have been mutated, yet have at least 80, 85, 90, 95, 96, 97, 98, or 99 percent identity in the VL domain with the VL regions depicted in the sequences described in Table 8. The present disclosure also provides antibodies or antibody fragments (e.g., antigen binding fragments) that specifically bind to DC-SIGN, the antibodies or antibody fragments (e.g., antigen binding fragments) comprise a VL CDR having an amino acid sequence of any one of the VL CDRs listed in Table 8. In particular, the invention provides antibodies or antibody fragments (e.g., antigen binding fragments) that specifically bind to DC-SIGN, which comprise (or alternatively, consist of) one, two, three or more VL CDRs having an amino acid sequence of any of the VL CDRs listed in Table 8.

TABLE 8
Sequences of exemplary anti-DC-SIGN monoclonal antibodies
>2B2Hz

| | HCDR1 | |
|---|---|---|
| SEQ ID NO: 1 | (Combined) | GYTFTNYGIN |
| | HCDR2 | |
| SEQ ID NO: 2 | (Combined) | YIYIGNDYTEYNERFKG |
| | HCDR3 | |
| SEQ ID NO: 3 | (Combined) | LYYGSSLYSYAMDY |
| | HCDR1 | |
| SEQ ID NO: 4 | (Kabat) | NYGIN |
| | HCDR2 | |
| SEQ ID NO: 2 | (Kabat) | YIYIGNDYTEYNERFKG |
| | HCDR3 | |
| SEQ ID NO: 3 | (Kabat) | LYYGSSLYSYAMDY |
| | HCDR1 | |
| SEQ ID NO: 5 | (Chothia) | GYTFTNY |
| | HCDR2 | |
| SEQ ID NO: 6 | (Chothia) | YIGNDY |
| | HCDR3 | |
| SEQ ID NO: 3 | (Chothia) | LYYGSSLYSYAMDY |
| | HCDR1 | |
| SEQ ID NO: 7 | (IMGT) | GYTFTNYG |
| | HCDR2 | |
| SEQ ID NO: 8 | (IMGT) | IYIGN |
| | HCDR3 | |
| SEQ ID NO: 9 | (IMGT) | ARLYYGSSLYSYAMDY |
| SEQ ID NO: | VH | QVQLVQSGAEVKKPGASVKVSCKASGYTFTNYGINWVRQAPGQRLEWMGY |
| 10 | | IYIGNDYTEYNERFKGRVTITSDTSASTAYMELSSLRSEDTAVYYCARLYYGSSLY |
| | | SYAMDYWGQGTTVTVSS |
| SEQ ID NO: | DNA VH | CAAGTTCAGTTGGTTCAGTCTGGCGCCGAAGTGAAGAAACCTGGCGCCTC |
| 11 | | TGTGAAGGTGTCCTGCAAGGCTTCTGGCTACACCTTTACCAACTACGGCAT |
| | | CAACTGGGTCCGACAGGCTCCTGGCCAGAGATTGGAGTGGATGGGCTAC |
| | | ATCTACATCGGCAACGACTACACCGAGTACAACGAGCGGTTCAAGGGCAG |
| | | AGTGACCATCACCTCTGACACCTCTGCCTCCACCGCCTACATGGAACTGTC |
| | | CAGCCTGAGATCTGAGGACACCGCCGTGTACTACTGCGCCAGGCTGTACT |
| | | ATGGCTCCTCCCTGTACAGCTATGCCATGGACTACTGGGGACAGGGCACA |
| | | ACCGTGACAGTGAGCTCC |
| SEQ ID NO: | Heavy | QVQLVQSGAEVKKPGASVKVSCKASGYTFTNYGNWVRQAPGQRLEWMGY |
| 12 | Chain | IYIGNDYTEYNERFKGRVTITSDTSASTAYMELSSLRSEDTAVYYCARLYYGSSLY |
| | | SYAMDYWGQGTTVTVSSASTKGPSVFPLAPSSKSTSGGTAALGCLVKDYFPC |
| | | PVTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPEV |
| | | TCVVVAVSHEDPEVKFNWYVDGVEVHNAKTKPREEQYNSTYRVVSVLTVLH |
| | | QDWLNGKEYKCKVSNKALAAPIEKTISKAKGQPREPQVYTLPPSREEMTKNQ |
| | | VSLTCLVKGFYPCDIAVEWESNGQPENNYKTTPPVLDSDGSFFLYSKLTVDKSR |
| | | WQQGNVFSCSVMHEALHNHYTQKSLSLSPGK |
| SEQ ID NO: | DNA Heavy | CAAGTTCAGTTGGTTCAGTCTGGCGCCGAAGTGAAGAAACCTGGCGCCTC |
| 13 | Chain | TGTGAAGGTGTCCTGCAAGGCTTCTGGCTACACCTTTACCAACTACGGCAT |
| | | CAACTGGGTCCGACAGGCTCCTGGCCAGAGATTGGAGTGGATGGGCTAC |
| | | ATCTACATCGGCAACGACTACACCGAGTACAACGAGCGGTTCAAGGGCAG |
| | | AGTGACCATCACCTCTGACACCTCTGCCTCCACCGCCTACATGGAACTGTC |
| | | CAGCCTGAGATCTGAGGACACCGCCGTGTACTACTGCGCCAGGCTGTACT |
| | | ATGGCTCCTCCCTGTACAGCTATGCCATGGACTACTGGGGACAGGGCACA |
| | | ACCGTGACAGTGAGCTCCGCTAGCACCAAGGGCCCAAGTGTGTTTCCCCT |
| | | GGCCCCCAGCAGCAAGTCTACTTCCGGCGGAACTGCTGCCCTGGGTTGCC |
| | | TGGTGAAGGACTACTTCCCCTGTCCCGTGACAGTGTCCTGGAACTCTGGG |
| | | GCTCTGACTTCCGGCGTGCACACCTTCCCCGCCGTGCTGCAGAGCAGCGG |
| | | CCTGTACAGCCTGAGCAGCGTGGTGACAGTGCCCTCCAGCTCTCTGGGAA |
| | | CCCAGACCTATATCTGCAACGTGAACCACAAGCCCAGCAACACCAAGGTG |
| | | GACAAGAGAGTGGAGCCCAAGAGCTGCGACAAGACCCACACCTGCCCCC |
| | | CCTGCCCAGCTCCAGAACTGCTGGGAGGGCCTTCCGTGTTCCTGTTCCCCC |
| | | CCAAGCCCAAGGACACCCTGATGATCAGCAGGACCCCCGAGGTGACCTGC |
| | | GTGGTGGTGGCCGTGTCCCACGAGGACCCAGAGGTGAAGTTCAACTGGT |
| | | ACGTGGACGGCGTGGAGGTGCACAACGCCAAGACCAAGCCCAGAGAGGA |
| | | GCAGTACAACAGCACCTACAGGGTGGTGTCCGTGCTGACCGTGCTGCACC |
| | | AGGACTGGCTGAACGGCAAAGAATACAAGTGCAAAGTCTCCAACAAGGC |
| | | CCTGGCTGCCCCAATCGAAAAGACAATCAGCAAGGCCAAGGGCCAGCCAC |
| | | GGGAGCCCCAGGTGTACACCCTGCCCCCCAGCCGGGAGGAGATGACCAA |
| | | GAACCAGGTGTCCCTGACCTGTCTGGTGAAGGGCTTCTACCCCTGTGATAT |
| | | CGCCGTGGAGTGGGAGAGCAACGGCCAGCCCGAGAACAACTACAAGACC |
| | | ACCCCCCCAGTGCTGGACAGCGACGGCAGCTTCTTCCTGTACAGCAAGCT |
| | | GACCGTGGACAAGTCCAGGTGGCAGCAGGGCAACGTGTTCAGCTGCAGC |
| | | GTGATGCACGAGGCCCTGCACAACCACTACACCCAGAAGTCCCTGAGCCT |
| | | GAGCCCCGGCAAG |
| SEQ ID NO: | LCDR1 | RSSKSLLHSSGNTYLY |
| 14 | (Combined) | |
| SEQ ID NO: | LCDR2 | RMSNLAS |
| 15 | (Combined) | |
| SEQ ID NO: | LCDR3 | MQHLEYPYT |
| 16 | (Combined) | |
| SEQ ID NO: | LCDR1 | RSSKSLLHSSGNTYLY |
| 14 | (Ka bat) | |
| SEQ ID NO: | LCDR2 | RMSNLAS |
| 15 | (Kabat) | |
| SEQ ID NO: | LCDR3 | MQHLEYPYT |
| 16 | (Kabat) | |
| SEQ ID NO: | LCDR1 | SKSLLHSSGNTY |
| 17 | (Chothia) | |
| SEQ ID NO: | LCDR2 | RMS |
| 18 | (Chothia) | |
| SEQ ID NO: | LCDR3 | HLEYPY |
| 19 | (Chothia) | |
| SEQ ID NO: | LCDR1 | KSLLHSSGNTY |
| 20 | (IMGT) | |
| SEQ ID NO: | LCDR2 | RMS |
| 18 | (IMGT) | |
| SEQ ID NO: | LCDR3 | MQHLEYPYT |
| 16 | (IMGT) | |
| SEQ ID NO: | VL | DIVMTQSPLSLPVTPGEPASISCRSSKSLLHSSGNTYLYWFLQKPGQSPQLLISR |
| 21 | | MSNLASGVPDRFSGSGSGTDFTLKISRVEAEDVGVYYCMQHLEYPYTFGGGT |
| | | KVEIK |
| SEQ ID NO: | DNA VL | GACATTGTGATGACCCAGTCTCCACTGAGCCTGCCTGTGACACCTGGCGA |
| 22 | | GCCTGCTTCCATCTCCTGCCGGTCCTCTAAGTCCCTGCTGCACTCTTCCGGC |
| | | AATACCTACCTGTACTGGTTCCTGCAGAAGCCCGGCCAGTCTCCTCAGCTG |
| | | CTGATCTCCAGAATGTCCAACCTGGCCTCTGGCGTGCCCGACAGATTTTCT |
| | | GGCTCTGGATCTGGCACCGACTTCACCCTGAAGATCTCTAGAGTGGAAGC |
| | | CGAGGACGTGGGCGTGTACTACTGTATGCAGCACCTGGAATACCCCTACA |
| | | CCTTCGGCGGAGGCACCAAGGTGGAAATCAAG |
| SEQ ID NO: | Light | DIVMTQSPLSLPVTPGEPASISCRSSKSLLHSSGNTYLYWFLQKPGQSPQLLISR |
| 23 | Chain | MSNLASGVPDRFSGSGSGTDFTLKISRVEAEDVGVYYCMQHLEYPYTFGGGT |
| | | KVEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVTKSF |
| | | NRGEC |
| | | GACATTGTGATGACCCAGTCTCCACTGAGCCTGCCTGTGACACCTGGCGA |
| | | GCCTGCTTCCATCTCCTGCCGGTCCTCTAAGTCCCTGCTGCACTCTTCCGGC |
| | | AATACCTACCTGTACTGGTTCCTGCAGAAGCCCGGCCAGTCTCCTCAGCTG |
| | | CTGATCTCCAGAATGTCCAACCTGGCCTCTGGCGTGCCCGACAGATTTTCT |
| | | GGCTCTGGATCTGGCACCGACTTCACCCTGAAGATCTCTAGAGTGGAAGC |
| | | CGAGGACGTGGGCGTGTACTACTGTATGCAGCACCTGGAATACCCCTACA |
| | | CCTTCGGCGGAGGCACCAAGGTGGAAATCAAGCGTACGGTGGCCGCTCCC |
| | | AGCGTGTTCATCTTCCCCCCCAGCGACGAGCAGCTGAAGAGTGGCACCGC |
| | | CAGCGTGGTGTGCCTGCTGAACAACTTCTACCCCCGGGAGGCCAAGGTGC |
| | | AGTGGAAGGTGGACAACGCCCTGCAGAGCGGCAACAGCCAGGAGAGCGT |
| | | CACCGAGCAGGACAGCAAGGACTCCACCTACAGCCTGAGCAGCACCCTGA |
| | | CCCTGAGCAAGGCCGACTACGAGAAGCATAAGGTGTACGCCTGCGAGGT |
| SEQ ID NO: | DNA Light | GACCCACCAGGGCCTGTCCAGCCCCGTGACCAAGAGCTTCAACAGGGGCG |
| 24 | Chain | |

960K03 N925

| | | |
|---|---|---|
| SEQ ID NO: | HCDR1 | GFSLSTGGMSVS |
| 25 | (Combined) | |
| SEQ ID NO: | HCDR2 | LIDWDDDKYYSTSLKT |
| 26 | (Combined) | |
| SEQ ID NO: | HCDR3 | AHSGSYFDF |
| 27 | (Combined) | |
| SEQ ID NO: | HCDR1 | TGGMSVS |
| 28 | (Kabat) | |
| SEQ ID NO: | HCDR2 | LIDWDDDKYYSTSLKT |
| 26 | (Kabat) | |
| SEQ ID NO: | HCDR3 | AHSGSYFDF |
| 27 | (Kabat) | |
| SEQ ID NO: | HCDR1 | GFSLSTGGM |
| 29 | (Chothia) | |
| SEQ ID NO: | HCDR2 | DWDDD |
| 30 | (Chothia) | |
| SEQ ID NO: | HCDR3 | AHSGSYFDF |
| 27 | (Chothia) | |
| SEQ ID NO: | HCDR1 | GFSLSTGGMS |
| 31 | (IMGT) | |
| SEQ ID NO: | HCDR2 | IDWDDDK |
| 32 | (IMGT) | |
| SEQ ID NO: | HCDR3 | ARAHSGSYFDF |
| 33 | (IMGT) | |
| SEQ ID NO: | VH | QVTLRESGPALVKPTQTLTLTCTFSGFSLSTGGMSVSWIRQPPGKALEWLALI |
| 34 | | DWDDDKYYSTSLKTRLTISKDTSKNQLVLTMTNMDPVDTATYYCARAHSGSY |
| | | FDFWGQGTLVTVSS |
| SEQ ID NO: | DNA VH | CAGGTCACCTTGAGGGAGTCTGGTCCTGCGCTGGTGAAACCCACACAGAC |
| 35 | | CCTCACACTGACCTGCACCTTCTCTGGGTTCTCACTCAGCACTGGTGGAAT |
| | | GAGTGTGAGCTGGATCCGTCAGCCCCCAGGGAAGGCCCTGGAGTGGCTT |
| | | GCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCA |
| SEQ ID NO: | Heavy | QVTLRESGPALVKPTQTLTLTCTFSGFSLSTGGMSVSWIRQPPGKALEWLALI |
| 36 | Chain | DWDDDKYYSTSLKTRLTISKDTSKNQLVLTMTNMDPVDTATYYCARAHSGSY |
| | | FDFWGQGTLVTVSSASTKGPSVFPLAPSSKSTSGGTAALGCLVKDYFPCPVTV |
| | | SWNSGALTSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLTVLHQDWL |
| | | NGKEYKCKVSNKALAAPIEKTISKAKGQPREPQVYTLPPSREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 37 | Chain | CCTCACACTGACCTGCACCTTCTCTGGGTTCTCACTCAGCACTGGTGGAAT |
| | | GAGTGTGAGCTGGATCCGTCAGCCCCCAGGGAAGGCCCTGGAGTGGCTT |
| | | GCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCAGCCTCCACCAAGGGCCCATCGGTGTTTCCCCTGGCCCCCAGCAGCAA |
| | | GTCTACTTCCGGCGGAACTGCTGCCCTGGGTTGCCTGGTGAAGGACTACTT |
| | | CCCCTGTCCCGTGACAGTGTCCTGGAACTCTGGGGCTCTGACTTCCGGCGT |
| | | GCACACCTTCCCCGCCGTGCTGCAGAGCAGCGGCCTGTACAGCCTGAGCA |
| | | GCGTGGTGACAGTGCCCTCCAGCTCTCTGGGAACCCAGACCTATATCTGCA |
| | | ACGTGAACCACAAGCCCAGCAACACCAAGGTGGACAAGAGAGTGGAGCC |
| | | CAAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: | LCDR1 | RASQRISNWLA |
| 38 | (Combined) | |
| SEQ ID NO: | LCDR2 | KASSLES |
| 39 | (Combined) | |
| SEQ ID NO: | LCDR3 | QQFSSYWT |
| 40 | (Combined) | |
| SEQ ID NO: | LCDR1 | RASQRISNWLA |
| 38 | (Kabat) | |
| SEQ ID NO: | LCDR2 | KASSLES |
| 39 | (Kabat) | |
| SEQ ID NO: | LCDR3 | QQFSSYWT |
| 40 | (Kabat) | |
| SEQ ID NO: | LCDR1 | SQRISNW |
| 41 | (Chothia) | |
| SEQ ID NO: | LCDR2 | KAS |
| 42 | (Chothia) | |
| SEQ ID NO: | LCDR3 | FSSYW |
| 43 | (Chothia) | |
| SEQ ID NO: | LCDR1 | QRISNW |
| 44 | (IMGT) | |
| SEQ ID NO: | LCDR2 | KAS |
| 42 | (IMGT) | |
| SEQ ID NO: | LCDR3 | QQFSSYWT |
| 40 | (IMGT) | |
| SEQ ID NO: | VL | DIQMTQSPSTLSASVGDRVTITCRASQRISNWLAWYQQKPGKAPKLLIYKASS |
| 45 | | LESGVPSRFSGSGSGTEFTLTISSLQPDDFATYYCQQFSSYWTFGQGTKVEIK |
| SEQ ID NO: | DNA VL | GACATCCAGATGACCCAGTCTCCTTCCACCCTGTCTGCATCTGTAGGAGAC |
| 46 | | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAGTAGTTATTGGACGTTCGGCCAAGGGACC |
| | | AAGGTGGAAATCAAA |
| SEQ ID NO: | Light | DIQMTQSPSTLSASVGDRVTITCRASQRISNWLAWYQQKPGKAPKLLIYKASS |
| 47 | Chain | LESGVPSRFSGSGSGTEFTLTISSLQPDDFATYYCQQFSSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GACATCCAGATGACCCAGTCTCCTTCCACCCTGTCTGCATCTGTAGGAGAC |
| 48 | Chain | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAGTAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

958N05 S93A

| | | |
|---|---|---|
| SEQ ID NO: | HCDR1 | GFSLSTSGISVS |
| 49 | (Combined) | |
| SEQ ID NO: | HCDR2 | LIDWDDDKYYSTSLKT |
| 26 | (Combined) | |
| SEQ ID NO: | HCDR3 | TPSGSYGRYFDL |
| 50 | (Combined) | |
| SEQ ID NO: | HCDR1 | TSGISVS |
| 51 | (Kabat) | |
| SEQ ID NO: | HCDR2 | LIDWDDDKYYSTSLKT |
| 26 | (Kabat) | |
| SEQ ID NO: | HCDR3 | TPSGSYGRYFDL |
| 50 | (Kabat) | |
| SEQ ID NO: | HCDR1 | GFSLSTSGI |
| 52 | (Chothia) | |
| SEQ ID NO: | HCDR2 | DWDDD |
| 30 | (Chothia) | |
| SEQ ID NO: | HCDR3 | TPSGSYGRYFDL |
| 50 | (Chothia) | |
| SEQ ID NO: | HCDR1 | GFSLSTSGIS |
| 53 | (IMGT) | |
| SEQ ID NO: | HCDR2 | IDWDDDK |
| 32 | (IMGT) | |
| SEQ ID NO: | HCDR3 | ARTPSGSYGRYFDL |
| 54 | (IMGT) | |
| SEQ ID NO: | VH | QVTLRESGPALVKPTQTLTLTCTFSGFSLSTSGISVSWIRQPPGKALEWLALID |
| 55 | | WDDDKYYSTSLKTRLTISKDTSKNQVVLTMTNMDPVDTATYYCARTPSGSYG |
| | | RYFDLWGRGTLVTVSS |
| SEQ ID NO: | DNA VH | CAGGTCACCTTGAGGGAGTCTGGTCCTGCGCTGGTGAAACCCACACAGAC |
| 56 | | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCA |
| SEQ ID NO: | Heavy | QVTLRESGPALVKPTQTLTLTCTFSGFSLSTSGISVSWIRQPPGKALEWLALID |
| 57 | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 58 | Chain | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RASQSISNWLA |
| 59 | (Combined) | |
| SEQ ID NO: | LCDR2 | KASSLES |
| 39 | (Combined) | |
| SEQ ID NO: | LCDR3 | QQYNAYWT |
| 60 | (Combined) | |
| SEQ ID NO: | LCDR1 | RASQSISNWLA |
| 59 | (Ka bat) | |
| SEQ ID NO: | LCDR2 | KASSLES |
| 39 | (Kabat) | |
| SEQ ID NO: | LCDR3 | QQYNAYWT |
| 60 | (Ka bat) | |
| SEQ ID NO: | LCDR1 | SQSISNW |
| 61 | (Chothia) | |
| SEQ ID NO: | LCDR2 | KAS |
| 42 | (Chothia) | |
| SEQ ID NO: | LCDR3 | YNAYW |
| 62 | (Chothia) | |
| SEQ ID NO: | LCDR1 | QSISNW |
| 63 | (IMGT) | |
| SEQ ID NO: | LCDR2 | KAS |
| 42 | (IMGT) | |
| SEQ ID NO: | LCDR3 | QQYNAYWT |
| 60 | (IMGT) | |
| SEQ ID NO: | VL | DIQLTQSPSTLSASVGDRVTITCRASQSISNWLAWYQQKPGKAPKLLIYKASSL |
| 64 | | ESGVPSRFTGSGSGTEFTLTISSLQPDDFATYYCQQYNAYWTFGQGTKVEIK |
| SEQ ID NO: | DNA VL | GACATCCAGTTGACCCAGTCTCCTTCCACCCTGTCTGCATCTGTAGGAGAC |
| 65 | | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | ACTTATTACTGCCAACAGTATAATGCCTATTGGACGTTCGGCCAAGGGACC |
| | | AAGGTGGAAATCAAA |
| SEQ ID NO: | Light | DIQLTQSPSTLSASVGDRVTITCRASQSISNWLAWYQQKPGKAPKLLIYKASSL |
| 66 | Chain | ESGVPSRFTGSGSGTEFTLTISSLQPDDFATYYCQQYNAYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GACATCCAGTTGACCCAGTCTCCTTCCACCCTGTCTGCATCTGTAGGAGAC |
| 67 | Chain | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | vACTTATTACTGCCAACAGTATAATGCCTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

960K03 N92Q

| | | |
|---|---|---|
| SEQ ID NO: | HCDR1 | GFSLSTGGMSVS |
| 25 | (Combined) | |
| SEQ ID NO: | HCDR2 | LIDWDDDKYYSTSLKT |
| 26 | (Combined) | |
| SEQ ID NO: | HCDR3 | AHSGSYFDF |
| 27 | (Combined) | |
| SEQ ID NO: | HCDR1 | TGGMSVS |
| 28 | (Kabat) | |
| SEQ ID NO: | HCDR2 | LIDWDDDKYYSTSLKT |
| 26 | (Kabat) | |
| SEQ ID NO: | HCDR3 | AHSGSYFDF |
| 27 | (Kabat) | |
| SEQ ID NO: | HCDR1 | GFSLSTGGM |
| 29 | (Chothia) | |
| SEQ ID NO: | HCDR2 | DWDDD |
| 30 | (Chothia) | |
| SEQ ID NO: | HCDR3 | AHSGSYFDF |
| 27 | (Chothia) | |
| SEQ ID NO: | HCDR1 | GFSLSTGGMS |
| 31 | (IMGT) | |
| SEQ ID NO: | HCDR2 | IDWDDDK |
| 32 | (IMGT) | |
| SEQ ID NO: | HCDR3 | ARAHSGSYFDF |
| 33 | (IMGT) | |
| SEQ ID NO: | DWDD | QVTLRESGPALVKPTQTLTLTCTFSGFSLSTGGMSVSWIRQPPGKALEWLALI |
| 34 | VH | DKYYSTSLKTRLTISKDTSKNQLVLTMTNMDPVDTATYYCARAHSGSY |
| | | FDFWGQGTLVTVSS |
| SEQ ID NO: | DNA VH | CAGGTCACCTTGAGGGAGTCTGGTCCTGCGCTGGTGAAACCCACACAGAC |
| 35 | | CCTCACACTGACCTGCACCTTCTCTGGGTTCTCACTCAGCACTGGTGGAAT |
| | | GAGTGTGAGCTGGATCCGTCAGCCCCCAGGGAAGGCCCTGGAGTGGCTT |
| | | vGCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCA |
| SEQ ID NO: | Heavy | QVTLRESGPALVKPTQTLTLTCTFSGFSLSTGGMSVSWIRQPPGKALEWLALI |
| 36 | Chain | DWDDDKYYSTSLKTRLTISKDTSKNQLVLTMTNMDPVDTATYYCARAHSGSY |
| | | FDFWGQGTLVTVSSASTKGPSVFPLAPSSKSTSGGTAALGCLVKDYFPCPVTV |
| | | SWNSGALTSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLTVLHQDWL |
| | | NGKEYKCKVSNKALAAPIEKTISKAKGQPREPQVYTLPPSREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 37 | Chain | CCTCACACTGACCTGCACCTTCTCTGGGTTCTCACTCAGCACTGGTGGAAT |
| | | vGAGTGTGAGCTGGATCCGTCAGCCCCCAGGGAAGGCCCTGGAGTGGCTT |
| | | GCACTCATTGATTGGGATGATGATAAATACTACAGCACATCTCTGAAGACC |
| | | AGGCTCACCATCTCCAAGGACACCTCCAAAAACCAGCTGGTCCTTACAATG |
| | | ACCAACATGGACCCTGTGGACACAGCCACGTATTATTGTGCACGGGCTCAT |
| | | AGTGGGAGCTACTTTGACTTCTGGGGCCAGGGAACCCTGGTCACCGTCTC |
| | | CTCAGCCTCCACCAAGGGCCCATCGGTGTTTCCCCTGGCCCCCAGCAGCAA |
| | | GTCTACTTCCGGCGGAACTGCTGCCCTGGGTTGCCTGGTGAAGGACTACTT |
| | | CCCCTGTCCCGTGACAGTGTCCTGGAACTCTGGGGCTCTGACTTCCGGCGT |
| | | GCACACCTTCCCCGCCGTGCTGCAGAGCAGCGGCCTGTACAGCCTGAGCA |
| | | GCGTGGTGACAGTGCCCTCCAGCTCTCTGGGAACCCAGACCTATATCTGCA |
| | | ACGTGAACCACAAGCCCAGCAACACCAAGGTGGACAAGAGAGTGGAGCC |
| | | CAAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: | LCDR1 | RASQRISNWLA |
| 38 | (Combined) | |
| SEQ ID NO: | LCDR2 | KASSLES |
| 39 | (Combined) | |
| SEQ ID NO: | LCDR3 | QQFQSYWT |
| 68 | (Combined) | |
| SEQ ID NO: | LCDR1 | RASQRISNWLA |
| 38 | (Kabat) | |
| SEQ ID NO: | LCDR2 | KASSLES |
| 39 | (Kabat) | |
| SEQ ID NO: | LCDR3 | QQFQSYWT |
| 68 | (Kabat) | |
| SEQ ID NO: | LCDR1 | SQRISNW |
| 41 | (Chothia) | |
| SEQ ID NO: | LCDR2 | KAS |
| 42 | (Chothia) | |
| SEQ ID NO: | LCDR3 | FQSYW |
| 69 | (Chothia) | |
| SEQ ID NO: | LCDR1 | QRISNW |
| 44 | (IMGT) | |
| SEQ ID NO: | LCDR2 | KAS |
| 42 | (IMGT) | |
| SEQ ID NO: | LCDR3 | QQFQSYWT |
| 68 | (IMGT) | |
| SEQ ID NO: | VL | DIQMTQSPSTLSASVGDRVTITCRASQRISNWLAWYQQKPGKAPKLLIYKASS |
| 70 | | LESGVPSRFSGSGSGTEFTLTISSLQPDDFATYYCQQFQSYWTFGQGTKVEIK |
| SEQ ID NO: | DNA VL | GACATCCAGATGACCCAGTCTCCTTCCACCCTGTCTGCATCTGTAGGAGAC |
| 71 | | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | vCGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTCAGAGTTATTGGACGTTCGGCCAAGGGACC |
| | | AAGGTGGAAATCAAA |
| SEQ ID NO: | Light | DIQMTQSPSTLSASVGDRVTITCRASQRISNWLAWYQQKPGKAPKLLIYKASS |
| 72 | Chain | LESGVPSRFSGSGSGTEFTLTISSLQPDDFATYYCQQFQSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GACATCCAGATGACCCAGTCTCCTTCCACCCTGTCTGCATCTGTAGGAGAC |
| 73 | Chain | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTCAGAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

952P16 N92Q

| | | |
|---|---|---|
| SEQ ID NO: | HCDR1 | GFSLSTSGVSVS |
| 74 | (Combined) | |
| SEQ ID NO: | HCDR2 | LIDWDDDKYYSTSLKT |
| 26 | (Combined) | |
| SEQ ID NO: | HCDR3 | TPSGSYGRYFDL |
| 50 | (Combined) | |
| SEQ ID NO: | HCDR1 | TSGVSVS |
| 75 | (Kabat) | |
| SEQ ID NO: | HCDR2 | LIDWDDDKYYSTSLKT |
| 26 | (Kabat) | |
| SEQ ID NO: | HCDR3 | TPSGSYGRYFDL |
| 50 | (Kabat) | |
| SEQ ID NO: | HCDR1 | GFSLSTSGV |
| 76 | (Chothia) | |
| SEQ ID NO: | HCDR2 | DWDDD |
| 30 | (Chothia) | |
| SEQ ID NO: | HCDR3 | TPSGSYGRYFDL |
| 50 | (Chothia) | |
| SEQ ID NO: | HCDR1 | GFSLSTSGVS |
| 77 | (IMGT) | |
| SEQ ID NO: | HCDR2 | IDWDDDK |
| 32 | (IMGT) | |
| SEQ ID NO: | HCDR3 | ARTPSGSYGRYFDL |
| 54 | (IMGT) | |
| SEQ ID NO: | VH | QVTLRESGPALVKPTQTLTLTCTFSGFSLSTSGVSVSWIRQPPGKALEWLALID |
| 78 | | WDDDKYYSTSLKTRLTISKDTSKNQVVLTMTNMDPVDTATYYCARTPSGSYG |
| | | RYFDLWGRGTLVTVSS |
| SEQ ID NO: | DNA VH | CAGGTCACCTTGAGGGAGTCTGGTCCTGCGCTGGTGAAACCCACACAGAC |
| 79 | | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: | Heavy | QVTLRESGPALVKPTQTLTLTCTFSGFSLSTSGVSVSWIRQPPGKALEWLALID |
| 80 | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 81 | Chain | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | vCCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RASQSISNWLA |
| 59 | (Combined) | |
| SEQ ID NO: | LCDR2 | KASSLES |
| 39 | (Combined) | |
| SEQ ID NO: | LCDR3 | QQYQSYWT |
| 82 | (Combined) | |
| SEQ ID NO: | LCDR1 | RASQSISNWLA |
| 59 | (Kabat) | |
| SEQ ID NO: | LCDR2 | KASSLES |
| 39 | (Kabat) | |
| SEQ ID NO: | LCDR3 | QQYQSYWT |
| 82 | (Kabat) | |
| SEQ ID NO: | LCDR1 | SQSISNW |
| 61 | (Chothia) | |
| SEQ ID NO: | LCDR2 | KAS |
| 42 | (Chothia) | |
| SEQ ID NO: | LCDR3 | YQSYW |
| 83 | (Chothia) | |
| SEQ ID NO: | LCDR1 | QSISNW |
| 63 | (IMGT) | |
| SEQ ID NO: | LCDR2 | KAS |
| 42 | (IMGT) | |
| SEQ ID NO: | LCDR3 | QQYQSYWT |
| 82 | (IMGT) | |
| SEQ ID NO: | VL | DIQMTQSPSTLSASVGDRVTITCRASQSISNWLAWYQQKPGKAPKLLIYKASS |
| 84 | | LESGVPSRFSGSGSGTEFTLTISSLQPDDFATYYCQQYQSYWTFGQGTKVEI |
| SEQ ID NO: | DNA VL | GACATCCAGATGACCCAGTCTCCTTCCACCCTGTCTGCATCTGTAGGAGAC |
| 85 | | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAA |
| SEQ ID NO: | Light | DIQMTQSPSTLSASVGDRVTITCRASQSISNWLAWYQQKPGKAPKLLIYKASS |
| 86 | Chain | LESGVPSRFSGSGSGTEFTLTISSLQPDDFATYYCQQYQSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| | | GACATCCAGATGACCCAGTCTCCTTCCACCCTGTCTGCATCTGTAGGAGAC |
| | | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| SEQ ID NO: | DNA Light | ACTACGAGAAGCATAAGGTGTACGCCTGCGAGGTGACCCACCAGGGCCT |
| 87 | Chain | GTCCAGCCCCGTGACCAAGAGCTTCAACAGGGGCGAGTGC |

952G04 N92Q

| | | |
|---|---|---|
| SEQ ID NO: | HCDR1 | GFSLSTSGVSVT |
| 88 | (Combined) | |
| SEQ ID NO: | HCDR2 | LIDWDDDKYYSTSLKT |
| 26 | (Combined) | |
| SEQ ID NO: | HCDR3 | TPSGSYGRYFDL |
| 50 | (Combined) | |
| SEQ ID NO: | HCDR1 | TSGVSVT |
| 89 | (Kabat) | |
| SEQ ID NO: | HCDR2 | LIDWDDDKYYSTSLKT |
| 26 | (Kabat) | |
| SEQ ID NO: | HCDR3 | TPSGSYGRYFDL |
| 50 | (Kabat) | |
| SEQ ID NO: | HCDR1 | GFSLSTSGV |
| 76 | (Chothia) | |
| SEQ ID NO: | HCDR2 | DWDDD |
| 30 | (Chothia) | |
| SEQ ID NO: | HCDR3 | TPSGSYGRYFDL |
| 50 | (Chothia) | |
| SEQ ID NO: | HCDR1 | GFSLSTSGVS |
| 77 | (IMGT) | |
| SEQ ID NO: | HCDR2 | IDWDDDK |
| 32 | (IMGT) | |
| SEQ ID NO: | HCDR3 | ARTPSGSYGRYFDL |
| 54 | (IMGT) | |
| SEQ ID NO: | VH | QVTLRESGPALVKPTQTLTLTCTFSGFSLSTSGVSVTWIRQPPGKALEWLALID |
| 90 | | WDDDKYYSTSLKTRLAISKDTSKNQVVLTMTNMDPVDTATYYCARTPSGSYG |
| | | RYFDLWGRGTLVTVSS |
| SEQ ID NO: | DNA VH | CAGGTCACCTTGAGGGAGTCTGGTCCTGCGCTGGTGAAACCCACACAGAC |
| 91 | | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: | Heavy | QVTLRESGPALVKPTQTLTLTCTFSGFSLSTSGVSVTWIRQPPGKALEWLALID |
| 92 | Chain | WDDDKYYSTSLKTRLAISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 93 | Chain | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RASQSISTWLA |
| 94 | (Combined) | |
| SEQ ID NO: | LCDR2 | EASSLES |
| 95 | (Combined) | |
| SEQ ID NO: | LCDR3 | QQYQSYWT |
| 82 | (Combined) | |
| SEQ ID NO: | LCDR1 | RASQSISTWLA |
| 94 | (Kabat) | |
| SEQ ID NO: | LCDR2 | EASSLES |
| 95 | (Kabat) | |
| SEQ ID NO: | LCDR3 | QQYQSYWT |
| 82 | (Kabat) | |
| SEQ ID NO: | LCDR1 | SQSISTW |
| 96 | (Chothia) | |
| SEQ ID NO: | LCDR2 | EAS |
| 97 | (Chothia) | |
| SEQ ID NO: | LCDR3 | YQSYW |
| 83 | (Chothia) | |
| SEQ ID NO: | LCDR1 | QSISTW |
| 98 | (IMGT) | |
| SEQ ID NO: | LCDR2 | EAS |
| 97 | (IMGT) | |
| SEQ ID NO: | LCDR3 | QQYQSYWT |
| 82 | (IMGT) | |
| SEQ ID NO: | VL | DIQMTQSPSTLSASVGDRVTITCRASQSISTWLAWYQQKPGKAPKLLIYEASSL |
| 99 | | ESGVPSRFSGSGSGTEFTLTISSLQPDDFATYYCQQYQSYWTFGQGTKVEIK |
| SEQ ID NO: | DNA VL | GACATCCAGATGACCCAGTCTCCTTCCACCCTGTCTGCATCTGTAGGAGAC |
| 100 | | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAA |
| SEQ ID NO: | Light | DIQMTQSPSTLSASVGDRVTITCRASQSISTWLAWYQQKPGKAPKLLIYEASSL |
| 101 | Chain | ESGVPSRFSGSGSGTEFTLTISSLQPDDFATYYCQQYQSYWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | GACATCCAGATGACCCAGTCTCCTTCCACCCTGTCTGCATCTGTAGGAGAC |
| | | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATCAGAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| SEQ ID NO: | DNA Light | ACTACGAGAAGCATAAGGTGTACGCCTGCGAGGTGACCCACCAGGG |
| 102 | Chain | GTCCAGCCCCGTGACCAAGAGCTTCAACAGGGGCGAGTGCCCT |

232 Chimeric

| | | |
|---|---|---|
| SEQ ID NO: 1 | HCDR1 | GYTFTNYGIN |
| | (Combined) | |
| SEQ ID NO: 2 | HCDR2 | YIYIGNDYTEYNERFKG |
| | (Combined) | |
| SEQ ID NO: 3 | HCDR3 | LYYGSSLYSYAMDY |
| | (Combined) | |
| SEQ ID NO: 4 | HCDR1 | NYGIN |
| | (Kabat) | |
| SEQ ID NO: 2 | HCDR2 | YIYIGNDYTEYNERFKG |
| | (Kabat) | |
| SEQ ID NO: 3 | HCDR3 | LYYGSSLYSYAMDY |
| | (Kabat) | |
| SEQ ID NO: 5 | HCDR1 | GYTFTNY |
| | (Chothia) | |
| SEQ ID NO: 6 | HCDR2 | YIGNDY |
| | (Chothia) | |
| SEQ ID NO: 3 | HCDR3 | LYYGSSLYSYAMDY |
| | (Chothia) | |
| SEQ ID NO: 7 | HCDR1 | GYTFTNYG |
| | (IMGT) | |
| SEQ ID NO: 8 | HCDR2 | IYIGN |
| | (IMGT) | |
| SEQ ID NO: 9 | HCDR3 | ARLYYGSSLYSYAMDY |
| | (IMGT) | |
| SEQ ID NO: | VH | EVQLQQSGAELVRPGSSVKMSCKTSGYTFTNYGINWVKQRPGQGLEWIGYIY |
| 103 | | IGNDYTEYNERFKGKATLTSDTSSSTAHIQLNSLTSEDSAIYFCARLYYGSSLYSY |
| | | AMDYWGQGTSVTVSS |
| SEQ ID NO: | DNA VH | GAGGTTCAGCTGCAGCAGTCTGGAGCTGAGTTGGTGAGGCCTGGGTCCTC |
| 104 | | AGTGAAGATGTCCTGCAAGACTTCTGGATATACATTCACAAACTACGGTAT |
| | | AAACTGGGTGAAGCAGAGGCCTGGACAGGGCCTGGAATGGATTGGATAT |
| | | ATTTATATTGGAAATGATTATACTGAGTACAATGAGAGGTTCAAGGGCAA |
| | | GGCCACACTGACTTCAGACACATCCTCCAGCACAGCCCACATACAACTCAA |
| | | CAGCCTGACATCTGAGGACTCTGCAATCTATTTCTGTGCAAGACTTTACTAC |
| | | GGTAGTAGCCTCTATTCTTATGCTATGGACTACTGGGGTCAAGGAACCTCT |
| | | GTCACAGTCTCCTCT |
| SEQ ID NO: | Heavy | EVQLQQSGAELVRPGSSVKMSCKTSGYTFTNYGINWVKQRPGQGLEWIGYIY |
| 105 | Chain | IGNDYTEYNERFKGKATLTSDTSSSTAHIQLNSLTSEDSAIYFCARLYYGSSLYSY |
| | | AMDYWGQGTSVTVSSASTKGPSVFPLAPSSKSTSGGTAALGCLVKDYFPCPV |
| | | TVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVTC |
| | | VVVAVSHEDPEVKFNWYVDGVEVHNAKTKPREEQYNSTYRVVSVLIVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | GAGGTTCAGCTGCAGCAGTCTGGAGCTGAGTTGGTGAGGCCTGGGTCCTC |
| 106 | Chain | AGTGAAGATGTCCTGCAAGACTTCTGGATATACATTCACAAACTACGGTAT |
| | | AAACTGGGTGAAGCAGAGGCCTGGACAGGGCCTGGAATGGATTGGATAT |
| | | ATTTATATTGGAAATGATTATACTGAGTACAATGAGAGGTTCAAGGGCAA |
| | | GGCCACACTGACTTCAGACACATCCTCCAGCACAGCCCACATACAACTCAA |
| | | CAGCCTGACATCTGAGGACTCTGCAATCTATTTCTGTGCAAGACTTTACTAC |
| | | GGTAGTAGCCTCTATTCTTATGCTATGGACTACTGGGGTCAAGGAACCTCT |
| | | GTCACAGTCTCCTCTGCTAGCACCAAGGGCCCAAGTGTGTTTCCCCTGGCC |
| | | CCCAGCAGCAAGTCTACTTCCGGCGGAACTGCTGCCCTGGGTTGCCTGGT |
| | | GAAGGACTACTTCCCCTGTCCCGTGACAGTGTCCTGGAACTCTGGGGCTCT |
| | | GACTTCCGGCGTGCACACCTTCCCCGCCGTGCTGCAGAGCAGCGGCCTGT |
| | | ACAGCCTGAGCAGCGTGGTGACAGTGCCCTCCAGCTCTCTGGGAACCCAG |
| | | ACCTATATCTGCAACGTGAACCACAAGCCCAGCAACACCAAGGTGGACAA |
| | | GAGAGTGGAGCCCAAGAGCTGCGACAAGACCCACACCTGCCCCCCCTGCC |
| | | CAGCTCCAGAACTGCTGGGAGGGCCTTCCGTGTTCCTGTTCCCCCCCAAGC |
| | | CCAAGGACACCCTGATGATCAGCAGGACCCCCGAGGTGACCTGCGTGGTG |
| | | GTGGCCGTGTCCCACGAGGACCCAGAGGTGAAGTTCAACTGGTACGTGG |
| | | ACGGCGTGGAGGTGCACAACGCCAAGACCAAGCCCAGAGAGGAGCAGTA |
| | | CAACAGCACCTACAGGGTGGTGTCCGTGCTGACCGTGCTGCACCAGGACT |
| | | GGCTGAACGGCAAAGAATACAAGTGCAAAGTCTCCAACAAGGCCCTGGCT |
| | | GCCCCAATCGAAAAGACAATCAGCAAGGCCAAGGGCCAGCCACGGGAGC |
| | | CCCAGGTGTACACCCTGCCCCCCAGCCGGGAGGAGATGACCAAGAACCAG |
| | | GTGTCCCTGACCTGTCTGGTGAAGGGCTTCTACCCCTGTGATATCGCCGTG |
| | | GAGTGGGAGAGCAACGGCCAGCCCGAGAACAACTACAAGACCACCCCCC |
| | | CAGTGCTGGACAGCGACGGCAGCTTCTTCCTGTACAGCAAGCTGACCGTG |
| | | GACAAGTCCAGGTGGCAGCAGGGCAACGTGTTCAGCTGCAGCGTGATGC |
| | | ACGAGGCCCTGCACAACCACTACACCCAGAAGTCCCTGAGCCTGAGCCCC |
| | | GGCAAG |
| SEQ ID NO: | LCDR1 | RSSKSLLHSSGNTYLY |
| 14 | (Combined) | |
| SEQ ID NO: | LCDR2 | RMSNLAS |
| 15 | (Combined) | |
| SEQ ID NO: | LCDR3 | MQHLEYPYT |
| 16 | (Combined) | |
| SEQ ID NO: | LCDR1 | RSSKSLLHSSGNTYLY |
| 14 | (Ka bat) | |
| SEQ ID NO: | LCDR2 | RMSNLAS |
| 15 | (Ka bat) | |
| SEQ ID NO: | LCDR3 | MQHLEYPYT |
| 16 | (Kabat) | |
| SEQ ID NO: | LCDR1 | SKSLLHSSGNTY |
| 17 | (Chothia) | |
| SEQ ID NO: | LCDR2 | RMS |
| 18 | (Chothia) | |
| SEQ ID NO: | LCDR3 | HLEYPY |
| 19 | (Chothia) | |
| SEQ ID NO: | LCDR1 | KSLLHSSGNTY |
| 20 | (IMGT) | |
| SEQ ID NO: | LCDR2 | RMS |
| 18 | (IMGT) | |
| SEQ ID NO: | LCDR3 | MQHLEYPYT |
| 16 | (IMGT) | |
| SEQ ID NO: | VL | DIVMTQAAPSVSVTPGESVSISCRSSKSLLHSSGNTYLYWFLQRPGQSPQLLIS |
| 107 | | RMSNLASGVPDRFSGSGSGTAFTLRISRVEAEDVGVYYCMQHLEYPYTFGGG |
| | | TKLELK |
| SEQ ID NO: | DNA VL | GATATTGTGATGACTCAGGCTGCACCCTCTGTATCTGTCACTCCTGGAGAG |
| 108 | | TCAGTATCCATCTCCTGCAGGTCTAGTAAGAGTCTCCTCCATAGTAGTGGC |
| | | AACACTTACTTGTATTGGTTCCTGCAGAGGCCAGGCCAGTCTCCTCAGCTC |
| | | CTGATATCTCGGATGTCCAACCTTGCCTCAGGAGTCCCAGACAGGTTCAGT |
| | | GGCAGTGGGTCAGGAACTGCTTTCACACTGAGAATCAGTAGAGTGGAGG |
| | | CTGAGGATGTGGGTGTTTATTATTGTATGCAACATCTAGAATATCCGTACA |
| | | CGTTCGGAGGGGGGACCAAGCTGGAGCTAAAA |
| SEQ ID NO: | Light | DIVMTQAAPSVSVTPGESVSISCRSSKSLLHSSGNTYLYWFLQRPGQSPQLLIS |
| 109 | Chain | RMSNLASGVPDRFSGSGSGTAFTLRISRVEAEDVGVYYCMQHLEYPYTFGGG |
| | | TKLELKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTKS |
| | | FNRGEC |
| SEQ ID NO: | DNA Light | GATATTGTGATGACTCAGGCTGCACCCTCTGTATCTGTCACTCCTGGAGAG |
| 110 | Chain | TCAGTATCCATCTCCTGCAGGTCTAGTAAGAGTCTCCTCCATAGTAGTGGC |
| | | AACACTTACTTGTATTGGTTCCTGCAGAGGCCAGGCCAGTCTCCTCAGCTC |
| | | CTGATATCTCGGATGTCCAACCTTGCCTCAGGAGTCCCAGACAGGTTCAGT |
| | | GGCAGTGGGTCAGGAACTGCTTTCACACTGAGAATCAGTAGAGTGGAGG |
| | | CTGAGGATGTGGGTGTTTATTATTGTATGCAACATCTAGAATATCCGTACA |
| | | CGTTCGGAGGGGGGACCAAGCTGGAGCTAAAACGTACGGTGGCCGCTCC |
| | | CAGCGTGTTCATCTTCCCCCCCAGCGACGAGCAGCTGAAGAGTGGCACCG |
| | | CCAGCGTGGTGTGCCTGCTGAACAACTTCTACCCCCGGGAGGCCAAGGTG |
| | | CAGTGGAAGGTGGACAACGCCCTGCAGAGCGGCAACAGCCAGGAGAGC |
| | | GTCACCGAGCAGGACAGCAAGGACTCCACCTACAGCCTGAGCAGCACCCT |
| | | GACCCTGAGCAAGGCCGACTACGAGAAGCATAAGGTGTACGCCTGCGAG |
| | | GTGACCCACCAGGGCCTGTCCAGCCCCGTGACCAAGAGCTTCAACAGGGG |
| | | CGAGTGC |

960K03 Parental

| | | |
|---|---|---|
| SEQ ID NO: | HCDR1 | GFSLSTGGMCVS |
| 111 | (Combined) | |
| SEQ ID NO: | HCDR2 | LIDWDDDKYYSTSLKT |
| 26 | (Combined) | |
| SEQ ID NO: | HCDR3 | AHSGSYFDF |
| 27 | (Combined) | |
| SEQ ID NO: | HCDR1 | TGGMCVS |
| 112 | (Kabat) | |
| SEQ ID NO: | HCDR2 | LIDWDDDKYYSTSLKT |
| 26 | (Kabat) | |
| SEQ ID NO: | HCDR3 | AHSGSYFDF |
| 27 | (Kabat) | |
| SEQ ID NO: | HCDR1 | GFSLSTGGM |
| 29 | (Chothia) | |
| SEQ ID NO: | HCDR2 | DWDDD |
| 30 | (Chothia) | |
| SEQ ID NO: | HCDR3 | AHSGSYFDF |
| 27 | (Chothia) | |
| SEQ ID NO: | HCDR1 | GFSLSTGGMC |
| 113 | (IMGT) | |
| SEQ ID NO: | HCDR2 | IDWDDDK |
| 32 | (IMGT) | |
| SEQ ID NO: | HCDR3 | ARAHSGSYFDF |
| 33 | (IMGT) | |
| SEQ ID NO: | VH | QVTLRESGPALVKPTQTLTLTCTFSGFSLSTGGMCVSWIRQPPGKALEWLALI |
| 114 | | DWDDDKYYSTSLKTRLTISKDTSKNQLVLTMTNMDPVDTATYYCARAHSGSY |
| | | FDFWGQGTLVTVSS |
| SEQ ID NO: | DNA VH | CAGGTCACCTTGAGGGAGTCTGGTCCTGCGCTGGTGAAACCCACACAGAC |
| 115 | | CCTCACACTGACCTGCACCTTCTCTGGGTTCTCACTCAGCACTGGTGGAAT |
| | | GTGTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGCTGGTCCTTACAATGA |
| | | CCAACATGGACCCTGTGGACACAGCCACGTATTATTGTGCACGGGCTCATA |
| | | GTGGGAGCTACTTTGACTTCTGGGGCCAGGGAACCCTGGTCACCGTCTCC |
| | | TCA |
| SEQ ID NO: | Heavy | QVTLRESGPALVKPTQTLTLTCTFSGFSLSTGGMCVSWIRQPPGKALEWLALI |
| 116 | Chain | DWDDDKYYSTSLKTRLTISKDTSKNQLVLTMTNMDPVDTATYYCARAHSGSY |
| | | FDFWGQGTLVTVSSASTKGPSVFPLAPSSKSTSGGTAALGCLVKDYFPCPVTV |
| | | SWNSGALTSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLTVLHQDWL |
| | | NGKEYKCKVSNKALAAPIEKTISKAKGQPREPQVYTLPPSREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 117 | Chain | CCTCACACTGACCTGCACCTTCTCTGGGTTCTCACTCAGCACTGGTGGAAT |
| | | GTGTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGCTGGTCCTTACAATGA |
| | | CCAACATGGACCCTGTGGACACAGCCACGTATTATTGTGCACGGGCTCATA |
| | | GTGGGAGCTACTTTGACTTCTGGGGCCAGGGAACCCTGGTCACCGTCTCC |
| | | TCAGCCTCCACCAAGGGCCCATCGGTGTTTCCCCTGGCCCCCAGCAGCAAG |
| | | TCTACTTCCGGCGGAACTGCTGCCCTGGGTTGCCTGGTGAAGGACTACTTC |
| | | CCCTGTCCCGTGACAGTGTCCTGGAACTCTGGGGCTCTGACTTCCGGCGTG |
| | | CACACCTTCCCCGCCGTGCTGCAGAGCAGCGGCCTGTACAGCCTGAGCAG |
| | | CGTGGTGACAGTGCCCTCCAGCTCTCTGGGAACCCAGACCTATATCTGCAA |
| | | CGTGAACCACAAGCCCAGCAACACCAAGGTGGACAAGAGAGTGGAGCCC |
| | | AAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: | LCDR1 | RASQRISNWLA |
| 38 | (Combined) | |
| SEQ ID NO: | LCDR2 | KASSLES |
| 39 | (Combined) | |
| SEQ ID NO: | LCDR3 | QQFNSYWT |
| 118 | (Combined) | |
| SEQ ID NO: | LCDR1 | RASQRISNWLA |
| 38 | (Kabat) | |
| SEQ ID NO: | LCDR2 | KASSLES |
| 39 | (Kabat) | |
| SEQ ID NO: | LCDR3 | QQFNSYWT |
| 118 | (Kabat) | |
| SEQ ID NO: | LCDR1 | SQRISNW |
| 41 | (Chothia) | |
| SEQ ID NO: | LCDR2 | KAS |
| 42 | (Chothia) | |
| SEQ ID NO: | LCDR3 | FNSYW |
| 119 | (Chothia) | |
| SEQ ID NO: | LCDR1 | QRISNW |
| 44 | (IMGT) | |
| SEQ ID NO: | LCDR2 | KAS |
| 42 | (IMGT) | |
| SEQ ID NO: | LCDR3 | QQFNSYWT |
| 118 | (IMGT) | |
| SEQ ID NO: | VL | DIQMTQSPSTLSASVGDRVTITCRASQRISNWLAWYQQKPGKAPKLLIYKASS |
| 120 | | LESGVPSRFSGSGSGTEFTLTISSLQPDDFATYYCQQFNSYWTFGQGTKVEIK |
| SEQ ID NO: | DNA VL | GACATCCAGATGACCCAGTCTCCTTCCACCCTGTCTGCATCTGTAGGAGAC |
| 121 | | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAA |
| SEQ ID NO: | Light | DIQMTQSPSTLSASVGDRVTITCRASQRISNWLAWYQQKPGKAPKLLIYKASS |
| 122 | Chain | LESGVPSRFSGSGSGTEFTLTISSLQPDDFATYYCQQFNSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GACATCCAGATGACCCAGTCTCCTTCCACCCTGTCTGCATCTGTAGGAGAC |
| 123 | Chain | AGAGTCACCATCACTTGCCGGGCCAGTCAGAGAATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

958N05 Parental

| | | |
|---|---|---|
| SEQ ID NO: | HCDR1 | GFSLSTSGISVS |
| 49 | (Combined) | |
| SEQ ID NO: | HCDR2 | LIDWDDDKYYSTSLKT |
| 26 | (Combined) | |
| SEQ ID NO: | HCDR3 | TPSGSYGRYFDL |
| 50 | (Combined) | |
| SEQ ID NO: | HCDR1 | TSGISVS |
| 51 | (Kabat) | |
| SEQ ID NO: | HCDR2 | LIDWDDDKYYSTSLKT |
| 26 | (Kabat) | |
| SEQ ID NO: | HCDR3 | TPSGSYGRYFDL |
| 50 | (Kabat) | |
| SEQ ID NO: | HCDR1 | GFSLSTSGI |
| 52 | (Chothia) | |
| SEQ ID NO: | HCDR2 | DWDDD |
| 30 | (Chothia) | |
| SEQ ID NO: | HCDR3 | TPSGSYGRYFDL |
| 50 | (Chothia) | |
| SEQ ID NO: | HCDR1 | GFSLSTSGIS |
| 53 | (IMGT) | |
| SEQ ID NO: | HCDR2 | IDWDDDK |
| 32 | (IMGT) | |
| SEQ ID NO: | HCDR3 | ARTPSGSYGRYFDL |
| 54 | (IMGT) | |
| SEQ ID NO: | VH | QVTLRESGPALVKPTQTLTLTCTFSGFSLSTSGISVSWIRQPPGKALEWLALID |
| 55 | | WDDDKYYSTSLKTRLTISKDTSKNQVVLTMTNMDPVDTATYYCARTPSGSYG |
| | | RYFDLWGRGTLVTVSS |
| SEQ ID NO: | DNA VH | CAGGTCACCTTGAGGGAGTCTGGTCCTGCGCTGGTGAAACCCACACAGAC |
| 56 | | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCA |
| SEQ ID NO: | Heavy | QVTLRESGPALVKPTQTLTLTCTFSGFSLSTSGISVSWIRQPPGKALEWLALID |
| 57 | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 58 | Chain | CCTCACACTGACCTGCACCTTCTCTGGGTTCTCACTCAGCACAAGTGGAAT |
| | | ATCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTATGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RASQSISNWLA |
| 59 | (Combined) | |
| SEQ ID NO: | LCDR2 | KASSLES |
| 39 | (Combined) | |
| SEQ ID NO: | LCDR3 | QQYNSYWT |
| 124 | (Combined) | |
| SEQ ID NO: | LCDR1 | RASQSISNWLA |
| 59 | (Kabat) | |
| SEQ ID NO: | LCDR2 | KASSLES |
| 39 | (Kabat) | |
| SEQ ID NO: | LCDR3 | QQYNSYWT |
| 124 | (Kabat) | |
| SEQ ID NO: | LCDR1 | SQSISNW |
| 61 | (Chothia) | |
| SEQ ID NO: | LCDR2 | KAS |
| 42 | (Chothia) | |
| SEQ ID NO: | LCDR3 | YNSYW |
| 125 | (Chothia) | |
| SEQ ID NO: | LCDR1 | QSISNW |
| 63 | (IMGT) | |
| SEQ ID NO: | LCDR2 | KAS |
| 42 | (IMGT) | |
| SEQ ID NO: | LCDR3 | QQYNSYWT |
| 124 | (IMGT) | |
| SEQ ID NO: | VL | DIQLTQSPSTLSASVGDRVTITCRASQSISNWLAWYQQKPGKAPKLLIYKASSL |
| 126 | | ESGVPSRFTGSGSGTEFTLTISSLQPDDFATYYCQQYNSYWTFGQGTKVEIK |
| SEQ ID NO: | DNA VL | GACATCCAGTTGACCCAGTCTCCTTCCACCCTGTCTGCATCTGTAGGAGAC |
| 127 | | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAA |
| SEQ ID NO: | Light | DIQLTQSPSTLSASVGDRVTITCRASQSISNWLAWYQQKPGKAPKLLIYKASSL |
| 128 | Chain | ESGVPSRFTGSGSGTEFTLTISSLQPDDFATYYCQQYNSYWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| SEQ ID NO: | DNA Light | GACATCCAGTTGACCCAGTCTCCTTCCACCCTGTCTGCATCTGTAGGAGAC |
| 129 | Chain | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCGTCAAGGTTCACCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

952P16 Parental

| | | |
|---|---|---|
| SEQ ID NO: | HCDR1 | GFSLSTSGVSVS |
| 74 | (Combined) | |
| SEQ ID NO: | HCDR2 | LIDWDDDKYYSTSLKT |
| 26 | (Combined) | |
| SEQ ID NO: | HCDR3 | TPSGSYGRYFDL |
| 50 | (Combined) | |
| SEQ ID NO: | HCDR1 | TSGVSVS |
| 75 | (Kabat) | |
| SEQ ID NO: | HCDR2 | LIDWDDDKYYSTSLKT |
| 26 | (Kabat) | |
| SEQ ID NO: | HCDR3 | TPSGSYGRYFDL |
| 50 | (Kabat) | |
| SEQ ID NO: | HCDR1 | GFSLSTSGV |
| 76 | (Chothia) | |
| SEQ ID NO: | HCDR2 | DWDDD |
| 30 | (Chothia) | |
| SEQ ID NO: | HCDR3 | TPSGSYGRYFDL |
| 50 | (Chothia) | |
| SEQ ID NO: | HCDR1 | GFSLSTSGVS |
| 77 | (IMGT) | |
| SEQ ID NO: | HCDR2 | IDWDDDK |
| 32 | (IMGT) | |
| SEQ ID NO: | HCDR3 | ARTPSGSYGRYFDL |
| 54 | (IMGT) | |
| SEQ ID NO: | VH | QVTLRESGPALVKPTQTLTLTCTFSGFSLSTSGVSVSWIRQPPGKALEWLALID |
| 78 | | WDDDKYYSTSLKTRLTISKDTSKNQVVLTMTNMDPVDTATYYCARTPSGSYG |
| | | RYFDLWGRGTLVTVSS |
| SEQ ID NO: | DNA VH | CAGGTCACCTTGAGGGAGTCTGGTCCTGCGCTGGTGAAACCCACACAGAC |
| 79 | | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: | Heavy | QVTLRESGPALVKPTQTLTLTCTFSGFSLSTSGVSVSWIRQPPGKALEWLALID |
| 80 | Chain | WDDDKYYSTSLKTRLTISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSW |
| | | QQGNVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 81 | Chain | CCTCACACTGACCTGCACCTTCTCTGGGTTCTCACTCAGCACTAGTGGAGT |
| | | GTCTGTGAGTTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTACTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RASQSISNWLA |
| 59 | (Combined) | |
| SEQ ID NO: | LCDR2 | KASSLES |
| 39 | (Combined) | |
| SEQ ID NO: | LCDR3 | QQYNSYWT |
| 124 | (Combined) | |
| SEQ ID NO: | LCDR1 | RASQSISNWLA |
| 59 | (Kabat) | |
| SEQ ID NO: | LCDR2 | KASSLES |
| 39 | (Kabat) | |
| SEQ ID NO: | LCDR3 | QQYNSYWT |
| 124 | (Kabat) | |
| SEQ ID NO: | LCDR1 | SQSISNW |
| 61 | (Chothia) | |
| SEQ ID NO: | LCDR2 | KAS |
| 42 | (Chothia) | |
| SEQ ID NO: | LCDR3 | YNSYW |
| 125 | (Chothia) | |
| SEQ ID NO: | LCDR1 | QSISNW |
| 63 | (IMGT) | |
| SEQ ID NO: | LCDR2 | KAS |
| 42 | (IMGT) | |
| SEQ ID NO: | LCDR3 | QQYNSYWT |
| 124 | (IMGT) | |
| SEQ ID NO: | VL | DIQMTQSPSTLSASVGDRVTITCRASQSISNWLAWYQQKPGKAPKLLIYKASS |
| 130 | | LESGVPSRFSGSGSGTEFTLTISSLQPDDFATYYCQQYNSYWTFGQGTKVEIK |
| SEQ ID NO: | DNA VL | GACATCCAGATGACCCAGTCTCCTTCCACCCTGTCTGCATCTGTAGGAGAC |
| 131 | | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAA |
| SEQ ID NO: | Light | DIQMTQSPSTLSASVGDRVTITCRASQSISNWLAWYQQKPGKAPKLLIYKASS |
| 132 | Chain | LESGVPSRFSGSGSGTEFTLTISSLQPDDFATYYCQQYNSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GACATCCAGATGACCCAGTCTCCTTCCACCCTGTCTGCATCTGTAGGAGAC |
| 133 | Chain | AGAGTCACCATCACTTGCCGGGCCAGTCAGAGTATTAGTAACTGGTTGGC |
| | | CTGGTATCAGCAGAAACCAGGGAAAGCCCCTAAGCTCCTGATCTATAAGG |
| | | CGTCTAGTTTAGAAAGTGGGGTCCCATCAAGGTTCAGCGGCAGTGGATCT |
| | | vGGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

952G04 Parental

| | | |
|---|---|---|
| SEQ ID NO: | HCDR1 | GFSLSTSGVSVT |
| 88 | (Combined) | |
| SEQ ID NO: | HCDR2 | LIDWDDDKYYSTSLKT |
| 26 | (Combined) | |
| SEQ ID NO: | HCDR3 | TPSGSYGRYFDL |
| 50 | (Combined) | |
| SEQ ID NO: | HCDR1 | TSGVSVT |
| 89 | (Kabat) | |
| SEQ ID NO: | HCDR2 | LIDWDDDKYYSTSLKT |
| 26 | (Kabat) | |
| SEQ ID NO: | HCDR3 | TPSGSYGRYFDL |
| 50 | (Kabat) | |
| SEQ ID NO: | HCDR1 | GFSLSTSGV |
| 76 | (Chothia) | |
| SEQ ID NO: | HCDR2 | DWDDD |
| 30 | (Chothia) | |
| SEQ ID NO: | HCDR3 | TPSGSYGRYFDL |
| 50 | (Chothia) | |
| SEQ ID NO: | HCDR1 | GFSLSTSGVS |
| 77 | (IMGT) | |
| SEQ ID NO: | HCDR2 | IDWDDDK |
| 32 | (IMGT) | |
| SEQ ID NO: | HCDR3 | ARTPSGSYGRYFDL |
| 54 | (IMGT) | |
| SEQ ID NO: | VH | QVTLRESG PALVKPTQTLTLTCTFSGFSLSTSGVSVTWIRQPPGKALEWLALID |
| 90 | | WDDDKYYSTSLKTRLAISKDTSKNQVVLTMTNMDPVDTATYYCARTPSGSYG |
| | | RYFDLWGRGTLVTVSS |
| SEQ ID NO: | DNA VH | CAGGTCACCTTGAGGGAGTCTGGTCCTGCGCTGGTGAAACCCACACAGAC |
| 91 | | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCA |
| SEQ ID NO: | Heavy | QVTLRESGPALVKPTQTLTLTCTFSGFSLSTSGVSVTWIRQPPGKALEWLALID |
| 92 | Chain | WDDDKYYSTSLKTRLAISKDTSKNQVVLTMTNMDPVDTATYYCARTPSGSYG |
| | | RYFDLWGRGTLVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 93 | Chain | CCTCACACTGACCTGCACCTTCTCTGGGTTCTCACTCAGCACTAGTGGAGT |
| | | GTCTGTGACCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCGCCATCTCCAAGGACACCTCCAAAAACCAGGTGGTCCTTACAATGA |
| | | CCAACATGGACCCTGTGGACACAGCCACGTATTATTGTGCACGGACCCCTA |
| | | GTGGGAGCTACGGGCGATACTTCGATCTCTGGGGCCGTGGCACCCTGGTC |
| | | ACTGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RASQSISTWLA |
| 94 | (Combined) | |
| SEQ ID NO: | LCDR2 | EASSLES |
| 95 | (Combined) | |
| SEQ ID NO: | LCDR3 | QQYNSYWT |
| 124 | (Combined) | |
| SEQ ID NO: | LCDR1 | RASQSISTWLA |
| 94 | (Kabat) | |
| SEQ ID NO: | LCDR2 | EASSLES |
| 95 | (Kabat) | |
| SEQ ID NO: | LCDR3 | QQYNSYWT |
| 124 | (Kabat) | |
| SEQ ID NO: | LCDR1 | SQSISTW |
| 96 | (Chothia) | |
| SEQ ID NO: | LCDR2 | EAS |
| 97 | (Chothia) | |
| SEQ ID NO: | LCDR3 | YNSYW |
| 125 | (Chothia) | |
| SEQ ID NO: | LCDR1 | QSISTW |
| 98 | (IMGT) | |
| SEQ ID NO: | LCDR2 | EAS |
| 97 | (IMGT) | |
| SEQ ID NO: | LCDR3 | QQYNSYWT |
| 124 | (IMGT) | |
| SEQ ID NO: | VL | DIQMTQSPSTLSASVGDRVTITCRASQSISTWLAWYQQKPGKAPKLLIYEASSL |
| 134 | | ESGVPSRFSGSGSGTEFTLTISSLQPDDFATYYCQQYNSYWTFGQGTKVEIK |
| SEQ ID NO: | DNA VL | GACATCCAGATGACCCAGTCTCCTTCCACCCTGTCTGCATCTGTAGGAGAC |
| 135 | | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAA |
| SEQ ID NO: | Light | DIQMTQSPSTLSASVGDRVTITCRASQSISTWLAWYQQKPGKAPKLLIYEASSL |
| 136 | Chain | ESGVPSRFSGSGSGTEFTLTISSLQPDDFATYYCQQYNSYWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| SEQ ID NO: | DNA Light | GACATCCAGATGACCCAGTCTCCTTCCACCCTGTCTGCATCTGTAGGAGAC |
| 137 | Chain | AGAGTCACCATCACTTGCCGGGCCAGTCAGAGTATTAGTACCTGGTTGGC |
| | | CTGGTATCAGCAGAAACCAGGGAAAGCCCCTAAGCTCCTGATCTATGAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTATAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

892D15 Parental

| | | |
|---|---|---|
| SEQ ID NO: | HCDR1 | GFSPSTSGMSVS |
| 138 | (Combined) | |
| SEQ ID NO: | HCDR2 | LIDWDDDKYFSTSLKT |
| 139 | (Combined) | |
| SEQ ID NO: | HCDR3 | AHSGSYFDY |
| 140 | (Combined) | |
| SEQ ID NO: | HCDR1 | TSGMSVS |
| 141 | (Kabat) | |
| SEQ ID NO: | HCDR2 | LIDWDDDKYFSTSLKT |
| 139 | (Kabat) | |
| SEQ ID NO: | HCDR3 | AHSGSYFDY |
| 140 | (Kabat) | |
| SEQ ID NO: | HCDR1 | GFSPSTSGM |
| 142 | (Chothia) | |
| SEQ ID NO: | HCDR2 | DWDDD |
| 30 | (Chothia) | |
| SEQ ID NO: | HCDR3 | AHSGSYFDY |
| 140 | (Chothia) | |
| SEQ ID NO: | HCDR1 | GFSPSTSGMS |
| 143 | (IMGT) | |
| SEQ ID NO: | HCDR2 | IDWDDDK |
| 32 | (IMGT) | |
| SEQ ID NO: | HCDR3 | ARAHSGSYFDY |
| 144 | (IMGT) | |
| SEQ ID NO: | VH | QVTLRESGPALVKPTQTLTLTCTFSGFSPSTSGMSVSWIRQPPGKALEWLALID |
| 145 | | WDDDKYFSTSLKTRLTISKDTSKNQVVLTMTNMDPVDTATYYCARAHSGSYF |
| | | DYWGQGTLVTVSS |
| SEQ ID NO: | DNA VH | CAGGTCACCTTGAGGGAGTCTGGTCCTGCGCTGGTGAAACCCACACAGAC |
| 146 | | CCTCACACTGACCTGCACCTTCTCTGGGTTCTCACCCAGCACTAGTGGAAT |
| | | GTCTGTGAGCTGGATCCGTCAGCCCCCAGGGAAGGCCCTGGAGTGGCTTG |
| | | CACTTATTGATTGGGATGATGATAAATACTTTAGCACATCTCTGAAGACCA |
| | | GGCTCACCATCTCCAAGGACACCTCCAAAAACCAGGTGGTCCTTACAATGA |
| | | CCAACATGGACCCTGTAGACACAGCCACGTATTATTGTGCACGGGCCCATA |
| | | GTGGGAGCTACTTTGACTACTGGGGCCAGGGAACCCTGGTCACCGTCTCC |
| | | TCA |
| SEQ ID NO: | Heavy | QVTLRESGPALVKPTQTLTLTCTFSGFSPSTSGMSVSWIRQPPGKALEWLALID |
| 147 | Chain | WDDDKYFSTSLKTRLTISKDTSKNQVVLTMTNMDPVDTATYYCARAHSGSYF |
| | | DYWGQGTLVTVSSASTKGPSVFPLAPSSKSTSGGTAALGCLVKDYFPCPVTVS |
| | | WNSGALTSGVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISRTPEVTCVVV |
| | | AVSHEDPEVKFNWYVDGVEVHNAKTKPREEQYNSTYRVVSVLTVLHQDWLN |
| | | GKEYKCKVSNKALAAPIEKTISKAKGQPREPQVYTLPPSREEMTKNQVSLTCLV |
| | | KGFYPCDIAVEWESNGQPENNYKTTPPVLDSDGSFFLYSKLTVDKSRWQQGN |
| | | VFSCSVMHEALHNHYTQKSLSLSPGK |
| SEQ ID NO: | DNA Heavy | CAGGTCACCTTGAGGGAGTCTGGTCCTGCGCTGGTGAAACCCACACAGAC |
| 148 | Chain | CCTCACACTGACCTGCACCTTCTCTGGGTTCTCACCCAGCACTAGTGGAAT |
| | | GTCTGTGAGCTGGATCCGTCAGCCCCCAGGGAAGGCCCTGGAGTGGCTTG |
| | | CACTTATTGATTGGGATGATGATAAATACTTTAGCACATCTCTGAAGACCA |
| | | GGCTCACCATCTCCAAGGACACCTCCAAAAACCAGGTGGTCCTTACAATGA |
| | | CCAACATGGACCCTGTAGACACAGCCACGTATTATTGTGCACGGGCCCATA |
| | | GTGGGAGCTACTTTGACTACTGGGGCCAGGGAACCCTGGTCACCGTCTCC |
| | | TCAGCCTCCACCAAGGGCCCATCGGTGTTTCCCCTGGCCCCCAGCAGCAAG |
| | | TCTACTTCCGGCGGAACTGCTGCCCTGGGTTGCCTGGTGAAGGACTACTTC |
| | | CCCTGTCCCGTGACAGTGTCCTGGAACTCTGGGGCTCTGACTTCCGGCGTG |
| | | CACACCTTCCCCGCCGTGCTGCAGAGCAGCGGCCTGTACAGCCTGAGCAG |
| | | CGTGGTGACAGTGCCCTCCAGCTCTCTGGGAACCCAGACCTATATCTGCAA |
| | | CGTGAACCACAAGCCCAGCAACACCAAGGTGGACAAGAGAGTGGAGCCC |
| | | AAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: | LCDR1 | RASQSISNWLA |
| 59 | (Combined) | |
| SEQ ID NO: | LCDR2 | KASSLES |
| 39 | (Combined) | |
| SEQ ID NO: | LCDR3 | QQFNSYWT |
| 118 | (Combined) | |
| SEQ ID NO: | LCDR1 | RASQSISNWLA |
| 59 | (Kabat) | |
| SEQ ID NO: | LCDR2 | KASSLES |
| 39 | (Kabat) | |
| SEQ ID NO: | LCDR3 | QQFNSYWT |
| 118 | (Kabat) | |
| SEQ ID NO: | LCDR1 | SQSISNW |
| 61 | (Chothia) | |
| SEQ ID NO: | LCDR2 | KAS |
| 42 | (Chothia) | |
| SEQ ID NO: | LCDR3 | FNSYW |
| 119 | (Chothia) | |
| SEQ ID NO: | LCDR1 | QSISNW |
| 63 | (IMGT) | |
| SEQ ID NO: | LCDR2 | KAS |
| 42 | (IMGT) | |
| SEQ ID NO: | LCDR3 | QQFNSYWT |
| 118 | (IMGT) | |
| SEQ ID NO: | VL | DIQMTQSPSTLSASVGDRVTITCRASQSISNWLAWYQQKPGKAPKLLIYKASS |
| 149 | | LESGVPSRFSGSGSGTEFTLTISSLQPDDFATYYCQQFNSYWTFGQGTKVEIK |
| SEQ ID NO: | DNA VL | GACATCCAGATGACCCAGTCTCCTTCCACCCTGTCTGCATCTGTAGGAGAC |
| 150 | | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAA |
| SEQ ID NO: | Light | DIQMTQSPSTLSASVGDRVTITCRASQSISNWLAWYQQKPGKAPKLLIYKASS |
| 151 | Chain | LESGVPSRFSGSGSGTEFTLTISSLQPDDFATYYCQQFNSYWTFGQGTKVEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GACATCCAGATGACCCAGTCTCCTTCCACCCTGTCTGCATCTGTAGGAGAC |
| 152 | Chain | AGAGTCACCATCACTTGCCGGGCCAGTCAGAGTATTAGTAACTGGTTGGC |
| | | CTGGTATCAGCAGAAACCAGGGAAAGCCCCTAAACTCCTGATCTATAAGG |
| | | CGTCTAGTTTAGAAAGTGGGGTCCCATCAAGGTTCAGCGGCAGTGGATCT |
| | | GGGACAGAATTCACTCTCACCATCAGCAGCCTGCAGCCTGATGATTTTGCA |
| | | ACTTATTACTGCCAACAGTTTAATAGTTATTGGACGTTCGGCCAAGGGACC |
| | | AAGGTGGAAATCAAACGAACTGTGGCTGCACCAAGCGTGTTCATCTTCCC |
| | | CCCCAGCGACGAGCAGCTGAAGAGTGGCACCGCCAGCGTGGTGTGCCTG |
| | | CTGAACAACTTCTACCCCCGGGAGGCCAAGGTGCAGTGGAAGGTGGACA |
| | | ACGCCCTGCAGAGCGGCAACAGCCAGGAGAGCGTCACCGAGCAGGACAG |
| | | CAAGGACTCCACCTACAGCCTGAGCAGCACCCTGACCCTGAGCAAGGCCG |
| | | ACTACGAGAAGCATAAGGTGTACGCCTGCGAGGTGACCCACCAGGGCCT |
| | | GTCCAGCCCCGTGACCAAGAGCTTCAACAGGGGCGAGTGC |

914M09 Parental

| | | |
|---|---|---|
| SEQ ID NO: | HCDR1 | GGSISSYYWN |
| 153 | (Combined) | |
| SEQ ID NO: | HCDR2 | RIYTSGSTNYNPSLKS |
| 154 | (Combined) | |
| SEQ ID NO: | HCDR3 | DSGGFYYYYGMDV |
| 155 | (Combined) | |
| SEQ ID NO: | HCDR1 | SYYWN |
| 156 | (Kabat) | |
| SEQ ID NO: | HCDR2 | RIYTSGSTNYNPSLKS |
| 154 | (Kabat) | |
| SEQ ID NO: | HCDR3 | DSGGFYYYYGMDV |
| 155 | (Kabat) | |
| SEQ ID NO: | HCDR1 | GGSISSY |
| 157 | (Chothia) | |
| SEQ ID NO: | HCDR2 | YTSGS |
| 158 | (Chothia) | |
| SEQ ID NO: | HCDR3 | DSGGFYYYYGMDV |
| 155 | (Chothia) | |
| SEQ ID NO: | HCDR1 | GGSISSYY |
| 159 | (IMGT) | |
| SEQ ID NO: | HCDR2 | IYTSGST |
| 160 | (IMGT) | |
| SEQ ID NO: | HCDR3 | ARDSGGFYYYYGMDV |
| 161 | (IMGT) | |
| SEQ ID NO: | VH | QVQLQESGPGLVKPSETLSLTCAVSGGSISSYYWNLIRQPAGKGLEWIGRIYTS |
| 162 | | GSTNYNPSLKSRVTMSVDTSKNQFSLKLSSVTAADTAVYYCARDSGGFYYYYG |
| | | MDVWGQGTTVTVSS |
| SEQ ID NO: | DNA VH | CAGGTGCAGCTGCAGGAGTCGGGCCCAGGACTGGTGAAGCCTTCGGAGA |
| 163 | | CCCTGTCCCTCACCTGCGCTGTCTCTGGTGGCTCCATCAGTAGTTACTACTG |
| | | GAACTTAATCCGGCAGCCCGCCGGGAAGGGACTGGAGTGGATTGGGCGT |
| | | ATCTATACCAGTGGGAGCACCAACTACAACCCCTCCCTCAAGAGTCGAGTC |
| | | ACCATGTCAGTAGACACGTCCAAGAACCAGTTCTCCCTGAAGCTGAGCTCT |
| | | GTGACCGCCGCGGACACGGCCGTGTATTACTGTGCGAGAGACTCCGGGG |
| | | GGTTCTACTACTACTACGGTATGGACGTCTGGGGCCAAGGGACCACGGTC |
| | | ACCGTCTCCTCA |
| SEQ ID NO: | Heavy | QVQLQESGPGLVKPSETLSLTCAVSGGSISSYYWNLIRQPAGKGLEWIGRIYTS |
| 164 | Chain | GSTNYNPSLKSRVTMSVDTSKNQFSLKLSSVTAADTAVYYCARDSGGFYYYYG |
| | | MDVWGQGTTVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAGGTGCAGCTGCAGGAGTCGGGCCCAGGACTGGTGAAGCCTTCGGAGA |
| 165 | Chain | CCCTGTCCCTCACCTGCGCTGTCTCTGGTGGCTCCATCAGTAGTTACTACTG |
| | | GAACTTAATCCGGCAGCCCGCCGGGAAGGGACTGGAGTGGATTGGGCGT |
| | | ATCTATACCAGTGGGAGCACCAACTACAACCCCTCCCTCAAGAGTCGAGTC |
| | | ACCATGTCAGTAGACACGTCCAAGAACCAGTTCTCCCTGAAGCTGAGCTCT |
| | | GTGACCGCCGCGGACACGGCCGTGTATTACTGTGCGAGAGACTCCGGGG |
| | | GGTTCTACTACTACTACGGTATGGACGTCTGGGGCCAAGGGACCACGGTC |
| | | ACCGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RASQGISSYLA |
| 166 | (Combined) | |
| SEQ ID NO: | LCDR2 | AASTLQG |
| 167 | (Combined) | |
| SEQ ID NO: | LCDR3 | QQLNSYPWT |
| 168 | (Combined) | |
| SEQ ID NO: | LCDR1 | RASQGISSYLA |
| 166 | (Kabat) | |
| SEQ ID NO: | LCDR2 | AASTLQG |
| 167 | (Kabat) | |
| SEQ ID NO: | LCDR3 | QQLNSYPWT |
| 168 | (Kabat) | |
| SEQ ID NO: | LCDR1 | SQGISSY |
| 169 | (Chothia) | |
| SEQ ID NO: | LCDR2 | AAS |
| 170 | (Chothia) | |
| SEQ ID NO: | LCDR3 | LNSYPW |
| 171 | (Chothia) | |
| SEQ ID NO: | LCDR1 | QGISSY |
| 172 | (IMGT) | |
| SEQ ID NO: | LCDR2 | AASTLQGGVP |
| 173 | (IMGT) | |
| SEQ ID NO: | LCDR3 | QQLNSYPWT |
| 168 | (IMGT) | |
| SEQ ID NO: | VL | DIQLTQSPSFLSASVGDRVTITCRASQGISSYLAWYQQKPGKAPKLLIYAASTLQ |
| 174 | | GGVPSRFSGSGSGTEFTLTISSLQPEDFATYHCQQLNSYPWTFGQGTKVEIK |
| | | GACATCCAGTTGACCCAGTCTCCATCCTTCCTGTCTGCATCTGTAGGAGAC |
| | | AGAGTCACCATCACTTGCCGGGCCAGTCAGGGCATTAGCAGTTATTTAGCC |
| | | TGGTATCAGCAAAAACCAGGGAAAGCCCCTAAGCTCCTGATCTATGCTGC |
| | | ATCCACCTTGCAAGGTGGGGTCCCATCAAGGTTCAGCGGCAGTGGATCTG |
| | | GGACAGAATTCACTCTCACAATCAGCAGCCTGCAGCCTGAAGATTTTGCAA |
| SEQ ID NO: | DNA VL | CTTATCACTGTCAACAGCTTAATAGTTACCCGTGGACGTTCGGCCAAGGGA |
| 175 | | CCAAGGTGGAAATCAAA |
| SEQ ID NO: | Light | DIQLTQSPSFLSASVGDRVTITCRASQGISSYLAWYQQKPGKAPKLLIYAASTLQ |
| 176 | Chain | GGVPSRFSGSGSGTEFTLTISSLQPEDFATYHCQQLNSYPWTFGQGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | GACATCCAGTTGACCCAGTCTCCATCCTTCCTGTCTGCATCTGTAGGAGAC |
| | | AGAGTCACCATCACTTGCCGGGCCAGTCAGGGCATTAGCAGTTATTTAGCC |
| | | TGGTATCAGCAAAAACCAGGGAAAGCCCCTAAGCTCCTGATCTATGCTGC |
| | | ATCCACCTTGCAAGGTGGGGTCCCATCAAGGTTCAGCGGCAGTGGATCTG |
| | | GGACAGAATTCACTCTCACAATCAGCAGCCTGCAGCCTGAAGATTTTGCAA |
| | | CTTATCACTGTCAACAGCTTAATAGTTACCCGTGGACGTTCGGCCAAGGGA |
| | | CCAAGGTGGAAATCAAACGAACTGTGGCTGCACCAAGCGTGTTCATCTTC |
| | | CCCCCCAGCGACGAGCAGCTGAAGAGTGGCACCGCCAGCGTGGTGTGCCT |
| | | GCTGAACAACTTCTACCCCCGGGAGGCCAAGGTGCAGTGGAAGGTGGAC |
| | | AACGCCCTGCAGAGCGGCAACAGCCAGGAGAGCGTCACCGAGCAGGACA |
| | | GCAAGGACTCCACCTACAGCCTGAGCAGCACCCTGACCCTGAGCAAGGCC |
| SEQ ID NO: | DNA Light | GACTACGAGAAGCATAAGGTGTACGCCTGCGAGGTGACCCACCAGGGCC |
| 177 | Chain | TGTCCAGCCCCGTGACCAAGAGCTTCAACAGGGGCGAGTGC |

906C18 Parental

| | | |
|---|---|---|
| SEQ ID NO: | HCDR1 | GFTFNNYWMN |
| 178 | (Combined) | |
| SEQ ID NO: | HCDR2 | NIRQDGSEKYYVDSVKG |
| 179 | (Combined) | |
| SEQ ID NO: | HCDR3 | ERAYCSTTSCPDYSNDY |
| 180 | (Combined) | |
| SEQ ID NO: | HCDR1 | NYWMN |
| 181 | (Kabat) | |
| SEQ ID NO: | HCDR2 | NIRQDGSEKYYVDSVKG |
| 179 | (Kabat) | |
| SEQ ID NO: | HCDR3 | ERAYCSTTSCPDYSNDY |
| 180 | (Kabat) | |
| SEQ ID NO: | HCDR1 | GFTFNNY |
| 182 | (Chothia) | |
| SEQ ID NO: | HCDR2 | RQDGSE |
| 183 | (Chothia) | |
| SEQ ID NO: | HCDR3 | ERAYCSTTSCPDYSNDY |
| 180 | (Chothia) | |
| SEQ ID NO: | HCDR1 | GFTFNNYW |
| 184 | (IMGT) | |
| SEQ ID NO: | HCDR2 | IRQDGSEK |
| 185 | (IMGT) | |
| SEQ ID NO: | HCDR3 | ARERAYCSTTSCPDYSNDY |
| 186 | (IMGT) | |
| SEQ ID NO: | VH | EVQLVESGGGLVQPGGSLRLSCAASGFTFNNYWMNWVRQAPGKGLEWVA |
| 187 | | NIRQDGSEKYYVDSVKGRFTISRDNAKNSLFLQMNSLRAEDTAVYYCARERAY |
| | | CSTTSCPDYSNDYWGQGTLVTVSS |
| SEQ ID NO: | DNA VH | GAGGTGCAGCTGGTGGAGTCTGGGGGAGGCTTGGTCCAGCCTGGGGGGT |
| 188 | | CCCTGAGGCTCTCCTGTGCAGCCTCTGGATTCACCTTTAATAACTATTGGAT |
| | | GAACTGGGTCCGCCAGGCTCCAGGGAAGGGGCTGGAGTGGGTGGCCAAC |
| | | ATAAGACAAGATGGAAGTGAAAAATACTATGTGGACTCTGTGAAGGGCC |
| | | GATTCACCATCTCCAGAGACAACGCCAAGAACTCACTGTTTCTACAAATGA |
| | | ACAGCCTGAGAGCCGAGGACACGGCTGTATATTACTGTGCGAGAGAGAG |
| | | GGCCTATTGTAGTACTACCAGCTGCCCTGACTACAGTAATGACTACTGGGG |
| | | CCAGGGAACCCTGGTCACCGTCTCCTCA |
| SEQ ID NO: | Heavy | EVQLVESGGGLVQPGGSLRLSCAASGFTFNNYWMNWVRQAPGKGLEWVA |
| 189 | Chain | NIRQDGSEKYYVDSVKGRFTISRDNAKNSLFLQMNSLRAEDTAVYYCARERAY |
| | | CSTTSCPDYSNDYWGQGTLVTVSSASTKGPSVFPLAPSSKSTSGGTAALGCLV |
| | | KDYFPCPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVAVSHEDPEVKFNWYVDGVEVHNAKTKPREEQYNSTYRVVS |
| | | VLTVLHQDWLNGKEYKCKVSNKALAAPIEKTISKAKGQPREPQVYTLPPSREE |
| | | MTKNQVSLTCLVKGFYPCDIAVEWESNGQPENNYKTTPPVLDSDGSFFLYSKL |
| | | TVDKSRWQQGNVFSCSVMHEALHNHYTQKSLSLSPGK |
| SEQ ID NO: | DNA Heavy | GAGGTGCAGCTGGTGGAGTCTGGGGGAGGCTTGGTCCAGCCTGGGGGGT |
| 190 | Chain | CCCTGAGGCTCTCCTGTGCAGCCTCTGGATTCACCTTTAATAACTATTGGAT |
| | | GAACTGGGTCCGCCAGGCTCCAGGGAAGGGGCTGGAGTGGGTGGCCAAC |
| | | ATAAGACAAGATGGAAGTGAAAAATACTATGTGGACTCTGTGAAGGGCC |
| | | GATTCACCATCTCCAGAGACAACGCCAAGAACTCACTGTTTCTACAAATGA |
| | | ACAGCCTGAGAGCCGAGGACACGGCTGTATATTACTGTGCGAGAGAGAG |
| | | GGCCTATTGTAGTACTACCAGCTGCCCTGACTACAGTAATGACTACTGGGG |
| | | CCAGGGAACCCTGGTCACCGTCTCCTCAGCCTCCACCAAGGGCCCATCGGT |
| | | GTTTCCCCTGGCCCCCAGCAGCAAGTCTACTTCCGGCGGAACTGCTGCCCT |
| | | GGGTTGCCTGGTGAAGGACTACTTCCCCTGTCCCGTGACAGTGTCCTGGA |
| | | ACTCTGGGGCTCTGACTTCCGGCGTGCACACCTTCCCCGCCGTGCTGCAGA |
| | | GCAGCGGCCTGTACAGCCTGAGCAGCGTGGTGACAGTGCCCTCCAGCTCT |
| | | CTGGGAACCCAGACCTATATCTGCAACGTGAACCACAAGCCCAGCAACAC |
| | | CAAGGTGGACAAGAGAGTGGAGCCCAAGAGCTGCGACAAGACCCACACC |
| | | TGCCCCCCCTGCCCAGCTCCAGAACTGCTGGGAGGGCCTTCCGTGTTCCTG |
| | | TTCCCCCCCAAGCCCAAGGACACCCTGATGATCAGCAGGACCCCCGAGGT |
| | | GACCTGCGTGGTGGTGGCCGTGTCCCACGAGGACCCAGAGGTGAAGTTC |
| | | AACTGGTACGTGGACGGCGTGGAGGTGCACAACGCCAAGACCAAGCCCA |
| | | GAGAGGAGCAGTACAACAGCACCTACAGGGTGGTGTCCGTGCTGACCGT |
| | | GCTGCACCAGGACTGGCTGAACGGCAAAGAATACAAGTGCAAAGTCTCCA |
| | | ACAAGGCCCTGGCTGCCCCAATCGAAAAGACAATCAGCAAGGCCAAGGG |
| | | CCAGCCACGGGAGCCCCAGGTGTACACCCTGCCCCCCAGCCGGGAGGAG |
| | | ATGACCAAGAACCAGGTGTCCCTGACCTGTCTGGTGAAGGGCTTCTACCCC |
| | | TGTGATATCGCCGTGGAGTGGGAGAGCAACGGCCAGCCCGAGAACAACT |
| | | ACAAGACCACCCCCCCAGTGCTGGACAGCGACGGCAGCTTCTTCCTGTACA |
| | | GCAAGCTGACCGTGGACAAGTCCAGGTGGCAGCAGGGCAACGTGTTCAG |
| | | CTGCAGCGTGATGCACGAGGCCCTGCACAACCACTACACCCAGAAGTCCC |
| | | TGAGCCTGAGCCCCGGCAAG |
| SEQ ID NO: | LCDR1 | RASQTINNNLA |
| 191 | (Combined) | |
| SEQ ID NO: | LCDR2 | GASTGAT |
| 192 | (Combined) | |
| SEQ ID NO: | LCDR3 | QQYNNWPRGLT |
| 193 | (Combined) | |
| SEQ ID NO: | LCDR1 | RASQTINNNLA |
| 191 | (Kabat) | |
| SEQ ID NO: | LCDR2 | GASTGAT |
| 192 | (Kabat) | |
| SEQ ID NO: | LCDR3 | QQYNNWPRGLT |
| 193 | (Kabat) | |
| SEQ ID NO: | LCDR1 | SQTINNN |
| 194 | (Chothia) | |
| SEQ ID NO: | LCDR2 | GAS |
| 195 | (Chothia) | |
| SEQ ID NO: | LCDR3 | YNNWPRGL |
| 196 | (Chothia) | |
| SEQ ID NO: | LCDR1 | QTINNN |
| 197 | (IMGT) | |
| SEQ ID NO: | LCDR2 | GASTGATGIP |
| 198 | (IMGT) | |
| SEQ ID NO: | LCDR3 | QQYNNWPRGLT |
| 193 | (IMGT) | |
| SEQ ID NO: | VL | EIVMTQSPATLSVSPGERATLSCRASQTINNNLAWFQQKPGQTPRLLIYGAST |
| 199 | | GATGIPARFSGSGSGTEFTLTISSLQSEDFAVYYCQQYNNWPRGLTFGGGTKV |
| | | EIK |
| SEQ ID NO: | DNA VL | GAAATAGTGATGACGCAGTCTCCAGCCACCCTGTCTGTGTCTCCAGGGGA |
| 200 | | AAGAGCCACCCTCTCCTGCAGGGCCAGTCAGACTATTAACAACAACTTAGC |
| | | CTGGTTCCAGCAGAAACCTGGCCAGACTCCCAGGCTCCTCATCTATGGTGC |
| | | ATCCACCGGGGCCACTGGTATCCCAGCCAGGTTCAGTGGCAGTGGGTCTG |
| | | GGACAGAGTTCACTCTCACCATCAGCAGCCTGCAGTCTGAAGATTTTGCAG |
| | | TTTATTACTGTCAGCAGTATAATAACTGGCCTCGAGGACTCACTTTCGGCG |
| | | GAGGGACCAAGGTGGAGATCAAA |
| SEQ ID NO: | Light | EIVMTQSPATLSVSPGERATLSCRASQTINNNLAWFQQKPGQTPRLLIYGAST |
| 201 | Chain | GATGIPARFSGSGSGTEFTLTISSLQSEDFAVYYCQQYNNWPRGLTFGGGTKV |
| | | EIKRTVAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPVTKSFNR |
| | | GEC |
| SEQ ID NO: | DNA Light | GAAATAGTGATGACGCAGTCTCCAGCCACCCTGTCTGTGTCTCCAGGGGA |
| 202 | Chain | AAGAGCCACCCTCTCCTGCAGGGCCAGTCAGACTATTAACAACAACTTAGC |
| | | CTGGTTCCAGCAGAAACCTGGCCAGACTCCCAGGCTCCTCATCTATGGTGC |
| | | ATCCACCGGGGCCACTGGTATCCCAGCCAGGTTCAGTGGCAGTGGGTCTG |
| | | GGACAGAGTTCACTCTCACCATCAGCAGCCTGCAGTCTGAAGATTTTGCAG |
| | | TTTATTACTGTCAGCAGTATAATAACTGGCCTCGAGGACTCACTTTCGGCG |
| | | GAGGGACCAAGGTGGAGATCAAACGAACTGTGGCTGCACCAAGCGTGTT |
| | | CATCTTCCCCCCCAGCGACGAGCAGCTGAAGAGTGGCACCGCCAGCGTGG |
| | | TGTGCCTGCTGAACAACTTCTACCCCCGGGAGGCCAAGGTGCAGTGGAAG |
| | | GTGGACAACGCCCTGCAGAGCGGCAACAGCCAGGAGAGCGTCACCGAGC |
| | | AGGACAGCAAGGACTCCACCTACAGCCTGAGCAGCACCCTGACCCTGAGC |
| | | AAGGCCGACTACGAGAAGCATAAGGTGTACGCCTGCGAGGTGACCCACC |
| | | AGGGCCTGTCCAGCCCCGTGACCAAGAGCTTCAACAGGGGCGAGTGC |

956E02 Parental

| | | |
|---|---|---|
| SEQ ID NO: | HCDR1 | GYTFTGYYIN |
| 203 | (Combined) | |
| SEQ ID NO: | HCDR2 | WINPNSGDTNYAQKFQG |
| 204 | (Combined) | |
| SEQ ID NO: | HCDR3 | ENSGYGKLFDY |
| 205 | (Combined) | |
| SEQ ID NO: | HCDR1 | GYYIN |
| 206 | (Kabat) | |
| SEQ ID NO: | HCDR2 | WINPNSGDTNYAQKFQG |
| 204 | (Kabat) | |
| SEQ ID NO: | HCDR3 | ENSGYGKLFDY |
| 205 | (Kabat) | |
| SEQ ID NO: | HCDR1 | GYTFTGY |
| 207 | (Chothia) | |
| SEQ ID NO: | HCDR2 | NPNSGD |
| 208 | (Chothia) | |
| SEQ ID NO: | HCDR3 | ENSGYGKLFDY |
| 205 | (Chothia) | |
| SEQ ID NO: | HCDR1 | GYTFTGYY |
| 209 | (IMGT) | |
| SEQ ID NO: | HCDR2 | INPNSGDT |
| 210 | (IMGT) | |
| SEQ ID NO: | HCDR3 | ARENSGYGKLFDY |
| 211 | (IMGT) | |
| SEQ ID NO: | VH | QVQLVQSGAEVKKPGASVKVSCKASGYTFTGYYINWVRQAPGQGLEWMG |
| 212 | | WINPNSGDTNYAQKFQGRVTMTRDPSISTAYMELSRLRSDDTAVYYCARENS |
| | | GYGKLFDYWGQGTLVTVSS |
| SEQ ID NO: | DNA VH | CAGGTGCAGCTGGTGCAGTCTGGGGCTGAGGTGAAGAAGCCTGGGGCCT |
| 213 | | CAGTGAAGGTCTCCTGCAAGGCTTCTGGATACACCTTCACCGGCTACTATA |
| | | TAAATTGGGTGCGACAGGCCCCTGGACAAGGGCTTGAGTGGATGGGATG |
| | | GATCAACCCTAACAGTGGTGACACAAACTATGCACAGAAGTTTCAGGGCA |
| | | GGGTCACCATGACCAGGGACCCGTCCATCAGCACAGCCTACATGGAGCTG |
| | | AGCAGGCTGAGATCTGACGACACGGCCGTGTATTACTGTGCGAGAGAGA |
| | | ATAGTGGCTACGGGAAGCTTTTTGACTACTGGGGCCAGGGAACCCTGGTC |
| | | ACCGTCTCCTCA |
| SEQ ID NO: | Heavy | QVQLVQSGAEVKKPGASVKVSCKASGYTFTGYYINWVRQAPGQGLEWMG |
| 214 | Chain | WINPNSGDTNYAQKFQGRVTMTRDPSISTAYMELSRLRSDDTAVYYCARENS |
| | | GYGKLFDYWGQGTLVTVSSASTKGPSVFPLAPSSKSTSGGTAALGCLVKDYFP |
| | | CPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPE |
| | | VTCVVVAVSHEDPEVKFNWYVDGVEVHNAKTKPREEQYNSTYRVVSVLTVL |
| | | HQDWLNGKEYKCKVSNKALAAPIEKTISKAKGQPREPQVYTLPPSREEMTKN |
| | | QVSLTCLVKGFYPCDIAVEWESNGQPENNYKTTPPVLDSDGSFFLYSKLTVDK |
| | | SRWQQGNVFSCSVMHEALHNHYTQKSLSLSPGK |
| SEQ ID NO: | DNA Heavy | CAGGTGCAGCTGGTGCAGTCTGGGGCTGAGGTGAAGAAGCCTGGGGCCT |
| 215 | Chain | CAGTGAAGGTCTCCTGCAAGGCTTCTGGATACACCTTCACCGGCTACTATA |
| | | TAAATTGGGTGCGACAGGCCCCTGGACAAGGGCTTGAGTGGATGGGATG |
| | | GATCAACCCTAACAGTGGTGACACAAACTATGCACAGAAGTTTCAGGGCA |
| | | GGGTCACCATGACCAGGGACCCGTCCATCAGCACAGCCTACATGGAGCTG |
| | | AGCAGGCTGAGATCTGACGACACGGCCGTGTATTACTGTGCGAGAGAGA |
| | | ATAGTGGCTACGGGAAGCTTTTTGACTACTGGGGCCAGGGAACCCTGGTC |
| | | ACCGTCTCCTCAGCCTCCACCAAGGGCCCATCGGTGTTTCCCCTGGCCCCC |
| | | AGCAGCAAGTCTACTTCCGGCGGAACTGCTGCCCTGGGTTGCCTGGTGAA |
| | | GGACTACTTCCCCTGTCCCGTGACAGTGTCCTGGAACTCTGGGGCTCTGAC |
| | | TTCCGGCGTGCACACCTTCCCCGCCGTGCTGCAGAGCAGCGGCCTGTACA |
| | | GCCTGAGCAGCGTGGTGACAGTGCCCTCCAGCTCTCTGGGAACCCAGACC |
| | | TATATCTGCAACGTGAACCACAAGCCCAGCAACACCAAGGTGGACAAGAG |
| | | AGTGGAGCCCAAGAGCTGCGACAAGACCCACACCTGCCCCCCCTGCCCAG |
| | | CTCCAGAACTGCTGGGAGGGCCTTCCGTGTTCCTGTTCCCCCCCAAGCCCA |
| | | AGGACACCCTGATGATCAGCAGGACCCCCGAGGTGACCTGCGTGGTGGT |
| | | GGCCGTGTCCCACGAGGACCCAGAGGTGAAGTTCAACTGGTACGTGGAC |
| | | GGCGTGGAGGTGCACAACGCCAAGACCAAGCCCAGAGAGGAGCAGTACA |
| | | ACAGCACCTACAGGGTGGTGTCCGTGCTGACCGTGCTGCACCAGGACTGG |
| | | CTGAACGGCAAAGAATACAAGTGCAAAGTCTCCAACAAGGCCCTGGCTGC |
| | | CCCAATCGAAAAGACAATCAGCAAGGCCAAGGGCCAGCCACGGGAGCCC |
| | | CAGGTGTACACCCTGCCCCCCAGCCGGGAGGAGATGACCAAGAACCAGG |
| | | TGTCCCTGACCTGTCTGGTGAAGGGCTTCTACCCCTGTGATATCGCCGTGG |
| | | AGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGG |
| | | ACAAGTCCAGGTGGCAGCAGGGCAACGTGTTCAGCTGCAGCGTGATGCA |
| | | CGAGGCCCTGCACAACCACTACACCCAGAAGTCCCTGAGCCTGAGCCCCG |
| | | GCAAG |
| SEQ ID NO: | LCDR1 | RSSQSLLHSNGYNYLD |
| 216 | (Combined) | |
| SEQ ID NO: | LCDR2 | LGSNRAS |
| 217 | (Combined) | |
| SEQ ID NO: | LCDR3 | MQALQTPYT |
| 218 | (Combined) | |
| SEQ ID NO: | LCDR1 | RSSQSLLHSNGYNYLD |
| 216 | (Kabat) | |
| SEQ ID NO: | LCDR2 | LGSNRAS |
| 217 | (Kabat) | |
| SEQ ID NO: | LCDR3 | MQALQTPYT |
| 218 | (Kabat) | |
| SEQ ID NO: | LCDR1 | SQSLLHSNGYNY |
| 219 | (Chothia) | |
| SEQ ID NO: | LCDR2 | LGS |
| 220 | (Chothia) | |
| SEQ ID NO: | LCDR3 | ALQTPY |
| 221 | (Chothia) | |
| SEQ ID NO: | LCDR1 | QSLLHSNGYNY |
| 222 | (IMGT) | |
| SEQ ID NO: | LCDR2 | LGS |
| 220 | (IMGT) | |
| SEQ ID NO: | LCDR3 | MQALQTPYT |
| 218 | (IMGT) | |
| SEQ ID NO: | VL | DIVMTQSPLSLPGTPGEPASISCRSSQSLLHSNGYNYLDWYLQKPGQSPQFLIY |
| 223 | | LGSNRASGVPDRFSGSGSGTDFTLKISRVEAEDVGVYYCMQALQTPYTFGQG |
| | | TKLEIK |
| SEQ ID NO: | DNA VL | GATATTGTGATGACTCAGTCTCCACTCTCCCTGCCCGGCACCCCTGGAGAG |
| 224 | | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGTTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATGCAAGCTCTACAAACTCCGTACA |
| | | CTTTTGGCCAGGGGACCAAGCTGGAGATCAAA |
| SEQ ID NO: | Light | DIVMTQSPLSLPGTPGEPASISCRSSQSLLHSNGYNYLDWYLQKPGQSPQFLIY |
| 225 | Chain | LGSNRASGVPDRFSGSGSGTDFTLKISRVEAEDVGVYYCMQALQTPYTFGQG |
| | | TKLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTKS |
| | | FNRGEC |
| SEQ ID NO: | DNA Light | GATATTGTGATGACTCAGTCTCCACTCTCCCTGCCCGGCACCCCTGGAGAG |
| 226 | Chain | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGTTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATGCAAGCTCTACAAACTCCGTACA |
| | | CTTTTGGCCAGGGGACCAAGCTGGAGATCAAACGAACTGTGGCTGCACCA |
| | | AGCGTGTTCATCTTCCCCCCCAGCGACGAGCAGCTGAAGAGTGGCACCGC |
| | | CAGCGTGGTGTGCCTGCTGAACAACTTCTACCCCCGGGAGGCCAAGGTGC |
| | | AGTGGAAGGTGGACAACGCCCTGCAGAGCGGCAACAGCCAGGAGAGCGT |
| | | CACCGAGCAGGACAGCAAGGACTCCACCTACAGCCTGAGCAGCACCCTGA |
| | | CCCTGAGCAAGGCCGACTACGAGAAGCATAAGGTGTACGCCTGCGAGGT |
| | | GACCCACCAGGGCCTGTCCAGCCCCGTGACCAAGAGCTTCAACAGGGGCG |
| | | AGTGC |

942K11 Parental

| | | |
|---|---|---|
| SEQ ID NO: | HCDR1 | GGSISSYYWT |
| 227 | (Combined) | |
| SEQ ID NO: | HCDR2 | RVFTSESTNYNPSLKN |
| 228 | (Combined) | |
| SEQ ID NO: | HCDR3 | DRGTYLGGFDP |
| 229 | (Combined) | |
| SEQ ID NO: | HCDR1 | SYYWT |
| 230 | (Kabat) | |
| SEQ ID NO: | HCDR2 | RVFTSESTNYNPSLKN |
| 228 | (Kabat) | |
| SEQ ID NO: | HCDR3 | DRGTYLGGFDP |
| 229 | (Kabat) | |
| SEQ ID NO: | HCDR1 | GGSISSY |
| 157 | (Chothia) | |
| SEQ ID NO: | HCDR2 | FTSES |
| 231 | (Chothia) | |
| SEQ ID NO: | HCDR3 | DRGTYLGGFDP |
| 229 | (Chothia) | |
| SEQ ID NO: | HCDR1 | GGSISSYY |
| 159 | (IMGT) | |
| SEQ ID NO: | HCDR2 | VFTSEST |
| 232 | (IMGT) | |
| SEQ ID NO: | HCDR3 | ARDRGTYLGGFDP |
| 233 | (IMGT) | |
| SEQ ID NO: | VH | QVQLQESGPGLVKPSETLSLTCTVSGGSISSYYWTWIRQPAGKGLEWIGRVFT |
| 234 | | SESTNYNPSLKNRVTMSVDTSKNQFSLRLNSVTAADTAMYYCARDRGTYLGG |
| | | FDPWGQGTLVTVSS |
| SEQ ID NO: | DNA VH | CAGGTGCAGCTGCAGGAGTCGGGCCCAGGACTGGTGAAGCCTTCGGAGA |
| 235 | | CCCTGTCCCTCACCTGCACTGTCTCTGGTGGCTCCATCAGTAGTTACTACTG |
| | | GACCTGGATCCGGCAGCCCGCCGGGAAGGGACTGGAGTGGATTGGGCGT |
| | | GTCTTTACCAGTGAGAGCACCAACTACAACCCCTCCCTCAAGAATCGAGTC |
| | | ACCATGTCAGTAGACACGTCCAAGAACCAGTTCTCCCTGAGGCTGAATTCT |
| | | GTGACCGCCGCGGACACGGCCATGTATTACTGTGCGAGAGACCGGGGGA |
| | | CCTACCTAGGGGGGTTCGACCCCTGGGGCCAGGGAACCCTGGTCACCGTC |
| | | TCCTCA |
| SEQ ID NO: | Heavy | QVQLQESGPGLVKPSETLSLTCTVSGGSISSYYWTWIRQPAGKGLEWIGRVFT |
| 236 | Chain | SESTNYNPSLKNRVTMSVDTSKNQFSLRLNSVTAADTAMYYCARDRGTYLGG |
| | | FDPWGQGTLVTVSSASTKGPSVFPLAPSSKSTSGGTAALGCLVKDYFPCPVTV |
| | | SWNSGALTSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISRTPEVTCVV |
| | | VAVSHEDPEVKFNWYVDGVEVHNAKTKPREEQYNSTYRVVSVLTVLHQDWL |
| | | NGKEYKCKVSNKALAAPIEKTISKAKGQPREPQVYTLPPSREEMTKNQVSLTCL |
| | | VKGFYPCDIAVEWESNGQPENNYKTTPPVLDSDGSFFLYSKLTVDKSRWQQG |
| | | NVFSCSVMHEALHNHYTQKSLSLSPGK |
| SEQ ID NO: | DNA Heavy | CAGGTGCAGCTGCAGGAGTCGGGCCCAGGACTGGTGAAGCCTTCGGAGA |
| 237 | Chain | CCCTGTCCCTCACCTGCACTGTCTCTGGTGGCTCCATCAGTAGTTACTACTG |
| | | GACCTGGATCCGGCAGCCCGCCGGGAAGGGACTGGAGTGGATTGGGCGT |
| | | GTCTTTACCAGTGAGAGCACCAACTACAACCCCTCCCTCAAGAATCGAGTC |
| | | ACCATGTCAGTAGACACGTCCAAGAACCAGTTCTCCCTGAGGCTGAATTCT |
| | | GTGACCGCCGCGGACACGGCCATGTATTACTGTGCGAGAGACCGGGGGA |
| | | CCTACCTAGGGGGGTTCGACCCCTGGGGCCAGGGAACCCTGGTCACCGTC |
| | | TCCTCAGCCTCCACCAAGGGCCCATCGGTGTTTCCCCTGGCCCCCAGCAGC |
| | | AAGTCTACTTCCGGCGGAACTGCTGCCCTGGGTTGCCTGGTGAAGGACTA |
| | | CTTCCCCTGTCCCGTGACAGTGTCCTGGAACTCTGGGGCTCTGACTTCCGG |
| | | CGTGCACACCTTCCCCGCCGTGCTGCAGAGCAGCGGCCTGTACAGCCTGA |
| | | GCAGCGTGGTGACAGTGCCCTCCAGCTCTCTGGGAACCCAGACCTATATCT |
| | | GCAACGTGAACCACAAGCCCAGCAACACCAAGGTGGACAAGAGAGTGGA |
| | | GCCCAAGAGCTGCGACAAGACCCACACCTGCCCCCCCTGCCCAGCTCCAG |
| | | AACTGCTGGGAGGGCCTTCCGTGTTCCTGTTCCCCCCCAAGCCCAAGGACA |
| | | CCCTGATGATCAGCAGGACCCCCGAGGTGACCTGCGTGGTGGTGGCCGTG |
| | | TCCCACGAGGACCCAGAGGTGAAGTTCAACTGGTACGTGGACGGCGTGG |
| | | AGGTGCACAACGCCAAGACCAAGCCCAGAGAGGAGCAGTACAACAGCAC |
| | | CTACAGGGTGGTGTCCGTGCTGACCGTGCTGCACCAGGACTGGCTGAACG |
| | | GCAAAGAATACAAGTGCAAAGTCTCCAACAAGGCCCTGGCTGCCCCAATC |
| | | GAAAAGACAATCAGCAAGGCCAAGGGCCAGCCACGGGAGCCCCAGGTGT |
| | | ACACCCTGCCCCCCAGCCGGGAGGAGATGACCAAGAACCAGGTGTCCCTG |
| | | ACCTGTCTGGTGAAGGGCTTCTACCCCTGTGATATCGCCGTGGAGTGGGA |
| | | GAGCAACGGCCAGCCCGAGAACAACTACAAGACCACCCCCCCAGTGCTGG |
| | | ACAGCGACGGCAGCTTCTTCCTGTACAGCAAGCTGACCGTGGACAAGTCC |
| | | AGGTGGCAGCAGGGCAACGTGTTCAGCTGCAGCGTGATGCACGAGGCCC |
| | | TGCACAACCACTACACCCAGAAGTCCCTGAGCCTGAGCCCCGGCAAG |
| SEQ ID NO: | LCDR1 | RSSQSLLHSNGYNYLD |
| 216 | (Combined) | |
| SEQ ID NO: | LCDR2 | LGSNRAS |
| 217 | (Combined) | |
| SEQ ID NO: | LCDR3 | IQALQTPFT |
| 238 | (Combined) | |
| SEQ ID NO: | LCDR1 | RSSQSLLHSNGYNYLD |
| 216 | (Kabat) | |
| SEQ ID NO: | LCDR2 | LGSNRAS |
| 217 | (Kabat) | |
| SEQ ID NO: | LCDR3 | IQALQTPFT |
| 238 | (Kabat) | |
| SEQ ID NO: | LCDR1 | SQSLLHSNGYNY |
| 219 | (Chothia) | |
| SEQ ID NO: | LCDR2 | LGS |
| 220 | (Chothia) | |
| SEQ ID NO: | LCDR3 | ALQTPF |
| 239 | (Chothia) | |
| SEQ ID NO: | LCDR1 | QSLLHSNGYNY |
| 222 | (IMGT) | |
| SEQ ID NO: | LCDR2 | LGS |
| 220 | (IMGT) | |
| SEQ ID NO: | LCDR3 | IQALQTPFT |
| 238 | (IMGT) | |
| SEQ ID NO: | VL | DIVMTQSPLSLPVTPGEPASISCRSSQSLLHSNGYNYLDWYLQKPGQSPQLLIY |
| 240 | | LGSNRASGVPDRFSGSGSGTDFTLKISRVEAEDVGVYYCIQALQTPFTFGQGT |
| | | KLEIK |
| SEQ ID NO: | DNA VL | GATATTGTGATGACTCAGTCTCCACTCTCCCTGCCCGTCACCCCTGGAGAG |
| 241 | | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGCTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATACAAGCTCTACAAACTCCGTTCA |
| | | CTTTTGGCCAGGGGACCAAACTGGAGATCAAA |
| SEQ ID NO: | Light | DIVMTQSPLSLPVTPGEPASISCRSSQSLLHSNGYNYLDWYLQKPGQSPQLLIY |
| 242 | Chain | LGSNRASGVPDRFSGSGSGTDFTLKISRVEAEDVGVYYCIQALQTPFTFGQGT |
| | | KLEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVTKSF |
| | | NRGEC |
| SEQ ID NO: | DNA Light | GATATTGTGATGACTCAGTCTCCACTCTCCCTGCCCGTCACCCCTGGAGAG |
| 243 | Chain | CCGGCCTCCATCTCCTGCAGGTCTAGTCAGAGCCTCCTGCATAGTAATGGA |
| | | TACAACTATTTGGATTGGTACCTGCAGAAGCCAGGGCAGTCTCCACAGCTC |
| | | CTGATCTATTTGGGTTCTAATCGGGCCTCCGGGGTCCCTGACAGGTTCAGT |
| | | GGCAGTGGATCAGGCACAGATTTTACACTGAAAATCAGCAGAGTGGAGG |
| | | CTGAGGATGTTGGGGTTTATTACTGCATACAAGCTCTACAAACTCCGTTCA |
| | | CTTTTGGCCAGGGGACCAAACTGGAGATCAAACGTACGGTGGCCGCTCCC |
| | | AGCGTGTTCATCTTCCCCCCCAGCGACGAGCAGCTGAAGAGTGGCACCGC |
| | | CAGCGTGGTGTGCCTGCTGAACAACTTCTACCCCCGGGAGGCCAAGGTGC |
| | | AGTGGAAGGTGGACAACGCCCTGCAGAGCGGCAACAGCCAGGAGAGCGT |
| | | CACCGAGCAGGACAGCAAGGACTCCACCTACAGCCTGAGCAGCACCCTGA |
| | | CCCTGAGCAAGGCCGACTACGAGAAGCATAAGGTGTACGCCTGCGAGGT |
| | | GACCCACCAGGGCCTGTCCAGCCCCGTGACCAAGAGCTTCAACAGGGGCG |
| | | AGTGC |

550E03 Parental

| | | |
|---|---|---|
| SEQ ID NO: | HCDR1 | GFSLSTSGMSVS |
| 244 | (Combined) | |
| SEQ ID NO: | HCDR2 | LIDWDDDKYYSTSLKT |
| 26 | (Combined) | |
| SEQ ID NO: | HCDR3 | MALRHAFDA |
| 245 | (Combined) | |
| SEQ ID NO: | HCDR1 | TSGMSVS |
| 141 | (Kabat) | |
| SEQ ID NO: | HCDR2 | LIDWDDDKYYSTSLKT |
| 26 | (Kabat) | |
| SEQ ID NO: | HCDR3 | MALRHAFDA |
| 245 | (Kabat) | |
| SEQ ID NO: | HCDR1 | GFSLSTSGM |
| 246 | (Chothia) | |
| SEQ ID NO: | HCDR2 | DWDDD |
| 30 | (Chothia) | |
| SEQ ID NO: | HCDR3 | MALRHAFDA |
| 245 | (Chothia) | |
| SEQ ID NO: | HCDR1 | GFSLSTSGMS |
| 247 | (IMGT) | |
| SEQ ID NO: | HCDR2 | IDWDDDK |
| 32 | (IMGT) | |
| SEQ ID NO: | HCDR3 | ARMALRHAFDA |
| 248 | (IMGT) | |
| SEQ ID NO: | VH | QVTLRESGPALVKPTQTLTLTCTFSGFSLSTSGMSVSWIRQPPGKALEWLALID |
| 249 | | WDDDKYYSTSLKTRLTISKDTSKNQVVLTMTNMDPVDTATYYCARMALRHA |
| | | FDAWGQGTMVTVSS |
| SEQ ID NO: | DNA VH | CAGGTCACCTTGAGGGAGTCTGGTCCTGCGCTGGTGAAGCCCACACAGAC |
| 250 | | CCTCACACTGACCTGCACCTTCTCTGGGTTCTCACTCAGCACTAGTGGAAT |
| | | GTCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGATGGCA |
| | | CTACGTCATGCTTTTGATGCCTGGGGCCAAGGGACAATGGTCACCGTCTCT |
| | | TCA |
| SEQ ID NO: | Heavy | QVTLRESGPALVKPTQTLTLTCTFSGFSLSTSGMSVSWIRQPPGKALEWLALID |
| 251 | Chain | WDDDKYYSTSLKTRLTISKDTSKNQVVLTMTNMDPVDTATYYCARMALRHA |
| | | FDAWGQGTMVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAGGTCACCTTGAGGGAGTCTGGTCCTGCGCTGGTGAAGCCCACACAGAC |
| 252 | Chain | CCTCACACTGACCTGCACCTTCTCTGGGTTCTCACTCAGCACTAGTGGAAT |
| | | GTCTGTGAGCTGGATCCGTCAGCCCCCAGGGAAGGCCCTGGAGTGGCTTG |
| | | CACTCATTGATTGGGATGATGATAAATACTACAGCACATCTCTGAAGACCA |
| | | GGCTCACCATCTCCAAGGACACCTCCAAAAACCAGGTGGTCCTTACAATGA |
| | | CCAACATGGACCCTGTGGACACAGCCACGTATTATTGTGCACGGATGGCA |
| | | CTACGTCATGCTTTTGATGCCTGGGGCCAAGGGACAATGGTCACCGTCTCT |
| | | TCAGCCTCCACCAAGGGCCCATCGGTGTTTCCCCTGGCCCCCAGCAGCAAG |
| | | TCTACTTCCGGCGGAACTGCTGCCCTGGGTTGCCTGGTGAAGGACTACTTC |
| | | CCCTGTCCCGTGACAGTGTCCTGGAACTCTGGGGCTCTGACTTCCGGCGTG |
| | | CACACCTTCCCCGCCGTGCTGCAGAGCAGCGGCCTGTACAGCCTGAGCAG |
| | | CGTGGTGACAGTGCCCTCCAGCTCTCTGGGAACCCAGACCTATATCTGCAA |
| | | CGTGAACCACAAGCCCAGCAACACCAAGGTGGACAAGAGAGTGGAGCCC |
| | | AAGAGCTGCGACAAGACCCACACCTGCCCCCCCTGCCCAGCTCCAGAACT |
| | | GCTGGGAGGGCCTTCCGTGTTCCTGTTCCCCCCCAAGCCCAAGGACACCCT |
| | | GATGATCAGCAGGACCCCCGAGGTGACCTGCGTGGTGGTGGCCGTGTCCC |
| | | ACGAGGACCCAGAGGTGAAGTTCAACTGGTACGTGGACGGCGTGGAGGT |
| | | GCACAACGCCAAGACCAAGCCCAGAGAGGAGCAGTACAACAGCACCTAC |
| | | AGGGTGGTGTCCGTGCTGACCGTGCTGCACCAGGACTGGCTGAACGGCA |
| | | AAGAATACAAGTGCAAAGTCTCCAACAAGGCCCTGGCTGCCCCAATCGAA |
| | | AAGACAATCAGCAAGGCCAAGGGCCAGCCACGGGAGCCCCAGGTGTACA |
| | | CCCTGCCCCCCAGCCGGGAGGAGATGACCAAGAACCAGGTGTCCCTGACC |
| | | TGTCTGGTGAAGGGCTTCTACCCCTGTGATATCGCCGTGGAGTGGGAGAG |
| | | CAACGGCCAGCCCGAGAACAACTACAAGACCACCCCCCCAGTGCTGGACA |
| | | GCGACGGCAGCTTCTTCCTGTACAGCAAGCTGACCGTGGACAAGTCCAGG |
| | | TGGCAGCAGGGCAACGTGTTCAGCTGCAGCGTGATGCACGAGGCCCTGC |
| | | ACAACCACTACACCCAGAAGTCCCTGAGCCTGAGCCCCGGCAAG |
| SEQ ID NO: | LCDR1 | TGSSSNIGAGYDVH |
| 253 | (Combined) | |
| SEQ ID NO: | LCDR2 | VNSNRPS |
| 254 | (Combined) | |
| SEQ ID NO: | LCDR3 | QSYDSSLSGWV |
| 255 | (Combined) | |
| SEQ ID NO: | LCDR1 | TGSSSNIGAGYDVH |
| 253 | (Kabat) | |
| SEQ ID NO: | LCDR2 | VNSNRPS |
| 254 | (Kabat) | |
| SEQ ID NO: | LCDR3 | QSYDSSLSGWV |
| 255 | (Kabat) | |
| SEQ ID NO: | LCDR1 | SSSNIGAGYD |
| 256 | (Chothia) | |
| SEQ ID NO: | LCDR2 | VNS |
| 257 | (Chothia) | |
| SEQ ID NO: | LCDR3 | YDSSLSGW |
| 258 | (Chothia) | |
| SEQ ID NO: | LCDR1 | SSNIGAGYD |
| 259 | (IMGT) | |
| SEQ ID NO: | LCDR2 | VNS |
| 257 | (IMGT) | |
| SEQ ID NO: | LCDR3 | QSYDSSLSGWV |
| 255 | (IMGT) | |
| SEQ ID NO: | VL | QSVLTQPPSVSGAPGQRVTISCTGSSSNIGAGYDVHWYQQLPGTAPKLLIYVN |
| 260 | | SNRPSGVPDRFSGSKSGTSASLAITGLQAEDEADYYCQSYDSSLSGWVFGGGT |
| | | KLTVL |
| SEQ ID NO: | DNA VL | CAGTCTGTGCTGACGCAGCCGCCCTCAGTGTCTGGGGCCCCAGGGCAGAG |
| 261 | | GGTCACCATCTCCTGCACTGGGAGCAGCTCCAACATCGGGGCAGGTTATG |
| | | ATGTACACTGGTACCAGCAGCTTCCAGGAACAGCCCCCAAACTCCTCATCT |
| | | ATGTTAACAGCAATCGGCCCTCAGGGGTCCCTGACCGATTCTCTGGCTCCA |
| | | AGTCTGGCACCTCAGCCTCCCTGGCCATCACTGGGCTCCAGGCTGAGGAT |
| | | GAGGCTGATTATTACTGCCAGTCCTATGACAGCAGCCTGAGTGGTTGGGT |
| | | GTTCGGCGGAGGGACCAAGTTGACCGTCCTA |
| SEQ ID NO: | Light | QSVLTQPPSVSGAPGQRVTISCTGSSSNIGAGYDVHWYQQLPGTAPKLLIYVN |
| 262 | Chain | SNRPSGVPDRFSGSKSGTSASLAITGLQAEDEADYYCQSYDSSLSGWVFGGGT |
| | | KLTVLGQPKAAPSVTLFPPSSEELQANKATLVCLISDFYPGAVIVAWKADSSPV |
| | | KAGVETTTPSKQSNNKYAASSYLSLTPEQWKSHRSYSCQVTHEGSTVEKTVAP |
| | | TECS |
| SEQ ID NO: | DNA Light | CAGTCTGTGCTGACGCAGCCGCCCTCAGTGTCTGGGGCCCCAGGGCAGAG |
| 263 | Chain | GGTCACCATCTCCTGCACTGGGAGCAGCTCCAACATCGGGGCAGGTTATG |
| | | ATGTACACTGGTACCAGCAGCTTCCAGGAACAGCCCCCAAACTCCTCATCT |
| | | ATGTTAACAGCAATCGGCCCTCAGGGGTCCCTGACCGATTCTCTGGCTCCA |
| | | AGTCTGGCACCTCAGCCTCCCTGGCCATCACTGGGCTCCAGGCTGAGGAT |
| | | GAGGCTGATTATTACTGCCAGTCCTATGACAGCAGCCTGAGTGGTTGGGT |
| | | GTTCGGCGGAGGGACCAAGTTGACCGTCCTAGGTCAGCCCAAGGCTGCCC |
| | | CCTCCGTGACCCTGTTCCCCCCCAGCTCCGAGGAACTGCAGGCCAACAAG |
| | | GCCACCCTGGTGTGCCTGATCAGCGACTTCTACCCTGGCGCCGTGACCGTG |
| | | GCCTGGAAGGCCGACAGCAGCCCCGTGAAGGCCGGCGTGGAGACAACCA |
| | | CCCCCAGCAAGCAGAGCAACAACAAGTACGCCGCCAGCAGCTACCTGAGC |
| | | CTGACCCCCGAGCAGTGGAAGAGCCACAGAAGCTACAGCTGCCAGGTCAC |
| | | CCACGAGGGCAGCACCGTGGAGAAAACCGTGGCCCCCACCGAGTGCAGC |

1G12 Hz

| | | |
|---|---|---|
| SEQ ID NO: | HCDR1 | GFSLTNYGVH |
| 264 | (Combined) | |
| SEQ ID NO: | HCDR2 | VIWRGESTDYNAAFMS |
| 265 | (Combined) | |
| SEQ ID NO: | HCDR3 | NGGSSGWYFDV |
| 266 | (Combined) | |
| SEQ ID NO: | HCDR1 | NYGVH |
| 267 | (Kabat) | |
| SEQ ID NO: | HCDR2 | VIWRGESTDYNAAFMS |
| 265 | (Kabat) | |
| SEQ ID NO: | HCDR3 | NGGSSGWYFDV |
| 266 | (Kabat) | |
| SEQ ID NO: | HCDR1 | GFSLTNY |
| 268 | (Chothia) | |
| SEQ ID NO: | HCDR2 | WRGES |
| 269 | (Chothia) | |
| SEQ ID NO: | HCDR3 | NGGSSGWYFDV |
| 266 | (Chothia) | |
| SEQ ID NO: | HCDR1 | GFSLTNYG |
| 270 | (IMGT) | |
| SEQ ID NO: | HCDR2 | IWRGEST |
| 271 | (IMGT) | |
| SEQ ID NO: | HCDR3 | ARNGGSSGWYFDV |
| 272 | (IMGT) | |
| SEQ ID NO: | VH | QVQLQESGPGLVKPSETLSLTCTVSGFSLTNYGVHWIRQPPGKGLEWIGVIW |
| 273 | | RGESTDYNAAFMSRVTISKDDSKSQVSLKLSSVTAADTAVYYCARNGGSSGW |
| | | YFDVWGQGTTVIVSS |
| SEQ ID NO: | DNA VH | CAAGTTCAGCTGCAAGAATCTGGCCCTGGCCTGGTCAAGCCTTCCGAGAC |
| 274 | | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGGCGGA |
| | | TCCTCCGGCTGGTACTTTGATGTGTGGGGCCAGGGCACCACCGTGACAGT |
| | | TAGTTCT |
| SEQ ID NO: | Heavy | QVQLQESGPGLVKPSETLSLTCTVSGFSLTNYGVHWIRQPPGKGLEWIGVIW |
| 275 | Chain | RGESTDYNAAFMSRVTISKDDSKSQVSLKLSSVTAADTAVYYCARNGGSSGW |
| | | YFDVWGQGTTVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAAGTTCAGCTGCAAGAATCTGGCCCTGGCCTGGTCAAGCCTTCCGAGAC |
| 276 | Chain | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGGCGGA |
| | | TCCTCCGGCTGGTACTTTGATGTGTGGGGCCAGGGCACCACCGTGACAGT |
| | | TAGTTCTGCTAGCACCAAGGGCCCAAGTGTGTTTCCCCTGGCCCCCAGCAG |
| | | CAAGTCTACTTCCGGCGGAACTGCTGCCCTGGGTTGCCTGGTGAAGGACT |
| | | ACTTCCCCTGTCCCGTGACAGTGTCCTGGAACTCTGGGGCTCTGACTTCCG |
| | | GCGTGCACACCTTCCCCGCCGTGCTGCAGAGCAGCGGCCTGTACAGCCTG |
| | | AGCAGCGTGGTGACAGTGCCCTCCAGCTCTCTGGGAACCCAGACCTATAT |
| | | CTGCAACGTGAACCACAAGCCCAGCAACACCAAGGTGGACAAGAGAGTG |
| | | GAGCCCAAGAGCTGCGACAAGACCCACACCTGCCCCCCCTGCCCAGCTCC |
| | | AGAACTGCTGGGAGGGCCTTCCGTGTTCCTGTTCCCCCCCAAGCCCAAGG |
| | | ACACCCTGATGATCAGCAGGACCCCCGAGGTGACCTGCGTGGTGGTGGCC |
| | | GTGTCCCACGAGGACCCAGAGGTGAAGTTCAACTGGTACGTGGACGGCG |
| | | TGGAGGTGCACAACGCCAAGACCAAGCCCAGAGAGGAGCAGTACAACAG |
| | | CACCTACAGGGTGGTGTCCGTGCTGACCGTGCTGCACCAGGACTGGCTGA |
| | | ACGGCAAAGAATACAAGTGCAAAGTCTCCAACAAGGCCCTGGCTGCCCCA |
| | | ATCGAAAAGACAATCAGCAAGGCCAAGGGCCAGCCACGGGAGCCCCAGG |
| | | TGTACACCCTGCCCCCCAGCCGGGAGGAGATGACCAAGAACCAGGTGTCC |
| | | CTGACCTGTCTGGTGAAGGGCTTCTACCCCTGTGATATCGCCGTGGAGTG |
| | | GGAGAGCAACGGCCAGCCCGAGAACAACTACAAGACCACCCCCCCAGTGC |
| | | TGGACAGCGACGGCAGCTTCTTCCTGTACAGCAAGCTGACCGTGGACAAG |
| | | TCCAGGTGGCAGCAGGGCAACGTGTTCAGCTGCAGCGTGATGCACGAGG |
| | | CCCTGCACAACCACTACACCCAGAAGTCCCTGAGCCTGAGCCCCGGCAAG |
| SEQ ID NO: | LCDR1 | KASQDVTSAVA |
| 277 | (Combined) | |
| SEQ ID NO: | LCDR2 | WTSTRHT |
| 278 | (Combined) | |
| SEQ ID NO: | LCDR3 | QQHYTTPLT |
| 279 | (Combined) | |
| SEQ ID NO: | LCDR1 | KASQDVTSAVA |
| 277 | (Kabat) | |
| SEQ ID NO: | LCDR2 | WTSTRHT |
| 278 | (Kabat) | |
| SEQ ID NO: | LCDR3 | QQHYTTPLT |
| 279 | (Kabat) | |
| SEQ ID NO: | LCDR1 | SQDVTSA |
| 280 | (Chothia) | |
| SEQ ID NO: | LCDR2 | WTS |
| 281 | (Chothia) | |
| SEQ ID NO: | LCDR3 | HYTTPL |
| 282 | (Chothia) | |
| SEQ ID NO: | LCDR1 | QDVTSA |
| 283 | (IMGT) | |
| SEQ ID NO: | LCDR2 | WTS |
| 281 | (IMGT) | |
| SEQ ID NO: | LCDR3 | QQHYTTPLT |
| 279 | (IMGT) | |
| SEQ ID NO: | VL | DIQLTQSPSFLSASVGDRVTITCKASQDVTSAVAWYQQKPGKAPKLLIYWTST |
| 284 | | RHTGVPSRFSGSGSGTEYTLTISSLQPEDFATYYCQQHYTTPLTFGQGTKLEIK |
| SEQ ID NO: | DNA VL | GATATTCAGCTGACCCAGTCTCCTAGCTTCCTGTCCGCTTCTGTGGGCGAC |
| 285 | | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAG |
| SEQ ID NO: | Light | DIQLTQSPSFLSASVGDRVTITCKASQDVTSAVAWYQQKPGKAPKLLIYWTST |
| 286 | Chain | RHTGVPSRFSGSGSGTEYTLTISSLQPEDFATYYCQQHYTTPLTFGQGTKLEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GATATTCAGCTGACCCAGTCTCCTAGCTTCCTGTCCGCTTCTGTGGGCGAC |
| 287 | Chain | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAGCGTACGGTGGCCGCTCCCAGCGTGTTCATCTTCC |
| | | CCCCCAGCGACGAGCAGCTGAAGAGTGGCACCGCCAGCGTGGTGTGCCT |
| | | GCTGAACAACTTCTACCCCCGGGAGGCCAAGGTGCAGTGGAAGGTGGAC |
| | | AACGCCCTGCAGAGCGGCAACAGCCAGGAGAGCGTCACCGAGCAGGACA |
| | | GCAAGGACTCCACCTACAGCCTGAGCAGCACCCTGACCCTGAGCAAGGCC |
| | | GACTACGAGAAGCATAAGGTGTACGCCTGCGAGGTGACCCACCAGGGCC |
| | | TGTCCAGCCCCGTGACCAAGAGCTTCAACAGGGGCGAGTGC |

1G12 mouse

| | | |
|---|---|---|
| SEQ ID NO: | HCDR1 | GFSLTNYGVH |
| 264 | (Combined) | |
| SEQ ID NO: | HCDR2 | VIWRGESTDYNAAFMS |
| 265 | (Combined) | |
| SEQ ID NO: | HCDR3 | NGGSSGWYFDV |
| 266 | (Combined) | |
| SEQ ID NO: | HCDR1 | NYGVH |
| 267 | (Kabat) | |
| SEQ ID NO: | HCDR2 | VIWRGESTDYNAAFMS |
| 265 | (Kabat) | |
| SEQ ID NO: | HCDR3 | NGGSSGWYFDV |
| 266 | (Kabat) | |
| SEQ ID NO: | HCDR1 | GFSLTNY |
| 268 | (Chothia) | |
| SEQ ID NO: | HCDR2 | WRGES |
| 269 | (Chothia) | |
| SEQ ID NO: | HCDR3 | NGGSSGWYFDV |
| 266 | (Chothia) | |
| SEQ ID NO: | HCDR1 | GFSLTNYG |
| 270 | (IMGT) | |
| SEQ ID NO: | HCDR2 | IWRGEST |
| 271 | (IMGT) | |
| SEQ ID NO: | HCDR3 | ARNGGSSGWYFDV |
| 272 | (IMGT) | |
| SEQ ID NO: | VH | QVQLQQSGPGLVQPSQSLSITCTVSGFSLTNYGVHWVRQSPGKGLEWLGVI |
| 288 | | WRGESTDYNAAFMSRLSVTKDDSKSQVFFKMNSLQADDTAIYYCARNGGSS |
| | | GWYFDVWGTGTTVTVSS |
| SEQ ID NO: | DNA VH | CAGGTGCAGCTACAGCAGTCAGGACCTGGCCTAGTGCAGCCCTCACAGAG |
| 289 | | CCTGTCCATAACCTGCACAGTCTCTGGTTTCTCATTAACTAACTATGGTGTA |
| | | CACTGGGTTCGCCAGTCTCCAGGAAAGGGTCTGGAGTGGCTGGGAGTGA |
| | | TATGGAGAGGTGAAAGCACAGACTACAATGCAGCTTTCATGTCCAGACTG |
| | | AGCGTCACCAAGGACGACTCCAAGAGCCAAGTTTTCTTTAAAATGAACAG |
| | | TCTGCAAGCTGATGACACTGCCATATACTACTGTGCCAGAAATGGCGGTA |
| | | GTAGCGGGTGGTACTTCGATGTCTGGGGCACAGGGACCACTGTCACCGTC |
| | | TCCTCA |
| SEQ ID NO: | Heavy | QVQLQQSGPGLVQPSQSLSITCTVSGFSLTNYGVHWVRQSPGKGLEWLGVI |
| 290 | Chain | WRGESTDYNAAFMSRLSVTKDDSKSQVFFKMNSLQADDTAIYYCARNGGSS |
| | | GWYFDVWGTGTTVTVSSAKTTAPSVYPLAPVCGGTTGSSVTLGCLVKGYFPC |
| | | PVTLTWNSGSLSSGVHTFPALLQSGLYTLSSSVTVTSNTWPSQTITCNVAHPA |
| | | SSTKVDKKIEPRVPITQNPCPPLKECPPCAAPDLLGGPSVFIFPPKIKDVLMISLS |
| | | PMVTCVVVAVSEDDPDVQISWFVNNVEVHTAQTQTHREDYNSTLRVVSALP |
| | | IQHQDWMSGKEFKCKVNNRALASPIEKTISKPRGPVRAPQVYVLPPPAEEMT |
| | | KKEFSLTCMITGFLPCEIAVDWTSNGRTEQNYKNTATVLDSDGSYFMYSKLRV |
| | | QKSTWERGSLFACSVVHEGLHNHLTTKTISRSLGK |
| SEQ ID NO: | DNA Heavy | CAGGTGCAGCTACAGCAGTCAGGACCTGGCCTAGTGCAGCCCTCACAGAG |
| 291 | Chain | CCTGTCCATAACCTGCACAGTCTCTGGTTTCTCATTAACTAACTATGGTGTA |
| | | CACTGGGTTCGCCAGTCTCCAGGAAAGGGTCTGGAGTGGCTGGGAGTGA |
| | | TATGGAGAGGTGAAAGCACAGACTACAATGCAGCTTTCATGTCCAGACTG |
| | | AGCGTCACCAAGGACGACTCCAAGAGCCAAGTTTTCTTTAAAATGAACAG |
| | | TCTGCAAGCTGATGACACTGCCATATACTACTGTGCCAGAAATGGCGGTA |
| | | GTAGCGGGTGGTACTTCGATGTCTGGGGCACAGGGACCACTGTCACCGTC |
| | | TCCTCAGCCAAAACAACAGCCCCATCGGTCTATCCACTGGCCCCTGTGTGT |
| | | GGAGGTACAACTGGCTCCTCGGTGACTCTAGGATGCCTGGTCAAGGGTTA |
| | | TTTCCCTTGTCCAGTGACCTTGACCTGGAACTCTGGATCCCTGTCCAGTGGT |
| | | GTGCACACCTTCCCAGCTCTCCTGCAGTCTGGCCTCTACACCCTCAGCAGCT |
| | | CAGTGACTGTAACCTCGAACACCTGGCCCAGCCAGACCATCACCTGCAATG |
| | | TGGCCCACCCGGCAAGCAGCACCAAAGTGGACAAGAAAATTGAGCCCAG |
| | | AGTGCCCATAACACAGAACCCCTGTCCTCCACTCAAAGAGTGTCCCCCATG |
| | | CGCAGCTCCAGACCTCTTGGGTGGACCATCCGTCTTCATCTTCCCTCCAAA |
| | | GATCAAGGATGTACTCATGATCTCCCTGAGCCCCATGGTCACATGTGTGGT |
| | | GGTGGCTGTGAGCGAGGATGACCCAGACGTCCAGATCAGCTGGTTTGTGA |
| | | ACAACGTGGAAGTACACACAGCTCAGACACAAACCCATAGAGAGGATTAC |
| | | AACAGTACTCTCCGGGTGGTCAGTGCCCTCCCCATCCAGCACCAGGACTG |
| | | GATGAGTGGCAAGGAGTTCAAATGCAAGGTCAACAACAGAGCCCTCGCAT |
| | | CCCCCATCGAGAAAACCATCTCAAAACCCAGAGGGCCAGTAAGAGCTCCA |
| | | CAGGTATATGTCTTGCCTCCACCAGCAGAAGAGATGACTAAGAAAGAGTT |
| | | CAGTCTGACCTGCATGATCACAGGCTTCTTACCTTGTGAAATTGCTGTGGA |
| | | vCTGGACCAGCAATGGGCGTACAGAGCAAAACTACAAGAACACCGCAACA |
| | | GTCCTGGACTCTGATGGTTCTTACTTCATGTACAGCAAGCTCAGAGTACAA |
| | | AAGAGCACTTGGGAAAGAGGAAGTCTTTTCGCCTGCTCAGTGGTCCACGA |
| | | GGGTCTGCACAATCACCTTACGACTAAGACCATCTCCCGGTCTCTGGGTAA |
| | | A |
| SEQ ID NO: | LCDR1 | KASQDVTSAVA |
| 277 | (Combined) | |
| SEQ ID NO: | LCDR2 | WTSTRHT |
| 278 | (Combined) | |
| SEQ ID NO: | LCDR3 | QQHYTTPLT |
| 279 | (Combined) | |
| SEQ ID NO: | LCDR1 | KASQDVTSAVA |
| 277 | (Kabat) | |
| SEQ ID NO: | LCDR2 | WTSTRHT |
| 278 | (Kabat) | |
| SEQ ID NO: | LCDR3 | QQHYTTPLT |
| 279 | (Kabat) | |
| SEQ ID NO: | LCDR1 | SQDVTSA |
| 280 | (Chothia) | |
| SEQ ID NO: | LCDR2 | WTS |
| 281 | (Chothia) | |
| SEQ ID NO: | LCDR3 | HYTTPL |
| 282 | (Chothia) | |
| SEQ ID NO: | LCDR1 | QDVTSA |
| 283 | (IMGT) | |
| SEQ ID NO: | LCDR2 | WTS |
| 281 | (IMGT) | |
| SEQ ID NO: | LCDR3 | QQHYTTPLT |
| 279 | (IMGT) | |
| SEQ ID NO: | VL | DIQMTQTHKFMSTSVGDRVSITCKASQDVTSAVAWYQQTPGQSPNLLIYWT |
| 292 | | STRHTGVPDRFTGSGSGTDYTLTISSVQAEDLALYYCQQHYTTPLTFGAGTKLE |
| | | LK |
| SEQ ID NO: | DNA VL | GACATTCAGATGACCCAGACTCACAAATTCATGTCCACATCAGTAGGAGAC |
| 293 | | AGGGTCAGCATCACCTGCAAGGCCAGTCAGGATGTGACTTCTGCTGTAGC |
| | | CTGGTATCAACAAACACCAGGACAATCTCCTAATCTACTGATTTACTGGAC |
| | | ATCCACCCGGCACACTGGAGTCCCTGATCGCTTCACAGGCAGTGGATCTG |
| | | GGACAGATTATACTCTCACCATCAGCAGTGTGCAGGCTGAAGACCTGGCA |
| | | CTTTATTACTGTCAGCAACATTATACCACTCCGCTCACGTTCGGTGCTGGGA |
| | | CCAAGCTGGAGCTAAAA |
| SEQ ID NO: | Light | DIQMTQTHKFMSTSVGDRVSITCKASQDVTSAVAWYQQTPGQSPNLLIYWT |
| 294 | Chain | STRHTGVPDRFTGSGSGTDYTLTISSVQAEDLALYYCQQHYTTPLTFGAGTKLE |
| | | LKRADAAPTVSIFPPSSEQLTSGGASVVCFLNNFYPKDINVKWKIDGSERQNG |
| | | VLNSWTDQDSKDSTYSMSSTLTLTKDEYERHNSYTCEATHKTSTSPIVKSFNR |
| | | NEC |
| SEQ ID NO: | DNA Light | GACATTCAGATGACCCAGACTCACAAATTCATGTCCACATCAGTAGGAGAC |
| 295 | Chain | AGGGTCAGCATCACCTGCAAGGCCAGTCAGGATGTGACTTCTGCTGTAGC |
| | | CTGGTATCAACAAACACCAGGACAATCTCCTAATCTACTGATTTACTGGAC |
| | | ATCCACCCGGCACACTGGAGTCCCTGATCGCTTCACAGGCAGTGGATCTG |
| | | GGACAGATTATACTCTCACCATCAGCAGTGTGCAGGCTGAAGACCTGGCA |
| | | CTTTATTACTGTCAGCAACATTATACCACTCCGCTCACGTTCGGTGCTGGGA |
| | | CCAAGCTGGAGCTAAAACGTGCCGATGCTGCACCAACTGTATCCATCTTCC |
| | | CACCATCCAGTGAGCAGTTAACATCTGGAGGTGCCTCAGTCGTGTGCTTCT |
| | | TGAACAACTTCTACCCCAAAGACATCAATGTCAAGTGGAAGATTGATGGC |
| | | AGTGAACGACAAAATGGCGTCCTGAACAGTTGGACTGATCAGGACAGCA |
| | | AAGACAGCACCTACAGCATGAGCAGCACCCTCACGTTGACCAAGGACGAG |
| | | TATGAACGACATAACAGCTATACCTGTGAGGCCACTCACAAGACATCAACT |
| | | TCACCCATTGTCAAGAGCTTCAACAGGAATGAGTGT |

1G12

| | | |
|---|---|---|
| SEQ ID NO: | HCDR1 | GFSLTNYGVH |
| 264 | (Combined) | |
| SEQ ID NO: | HCDR2 | VIWRGESTDYNAAFMS |
| 265 | (Combined) | |
| SEQ ID NO: | HCDR3 | NAGSSGWYFDV |
| 296 | (Combined) | |
| SEQ ID NO: | HCDR1 | NYGVH |
| 267 | (Kabat) | |
| SEQ ID NO: | HCDR2 | VIWRGESTDYNAAFMS |
| 265 | (Kabat) | |
| SEQ ID NO: | HCDR3 | NAGSSGWYFDV |
| 296 | (Kabat) | |
| SEQ ID NO: | HCDR1 | GFSLTNY |
| 268 | (Chothia) | |
| SEQ ID NO: | HCDR2 | WRGES |
| 269 | (Chothia) | |
| SEQ ID NO: | HCDR3 | NAGSSGWYFDV |
| 296 | (Chothia) | |
| SEQ ID NO: | HCDR1 | GFSLTNYG |
| 270 | (IMGT) | |
| SEQ ID NO: | HCDR2 | IWRGEST |
| 271 | (IMGT) | |
| SEQ ID NO: | HCDR3 | ARNAGSSGWYFDV |
| 297 | (IMGT) | |
| SEQ ID NO: | VH | QVQLQESGPGLVKPSETLSLTCTVSGFSLTNYGVHWIRQPPGKGLEWIGVIW |
| 298 | | RGESTDYNAAFMSRVTISKDDSKSQVSLKLSSVTAADTAVYYCARNAGSSGW |
| | | YFDVWGQGTTVIVSS |
| SEQ ID NO: | DNA VH | CAAGTTCAGCTGCAAGAATCTGGCCCTGGCCTGGTCAAGCCTTCCGAGAC |
| 299 | | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGCTGGCT |
| | | CCTCCGGCTGGTACTTTGATGTGTGGGGCCAGGGCACCACCGTGACAGTT |
| | | AGTTCT |
| SEQ ID NO: | Heavy | QVQLQESGPGLVKPSETLSLTCTVSGFSLTNYGVHWIRQPPGKGLEWIGVIW |
| 300 | Chain | RGESTDYNAAFMSRVTISKDDSKSQVSLKLSSVTAADTAVYYCARNAGSSGW |
| | | YFDVWGQGTTVTVSSASTKGPSVFPLAPSSKSTSGGTAALGCLVKDYFPCPVT |
| | | VSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCV |
| | | VVAVSHEDPEVKFNWYVDGVEVHNAKTKPREEQYNSTYRVVSVLTVLHQD |
| | | WLNGKEYKCKVSNKALAAPIEKTISKAKGQPREPQVYTLPPSREEMTKNQVSL |
| | | TCLVKGFYPCDIAVEWESNGQPENNYKTTPPVLDSDGSFFLYSKLTVDKSRW |
| | | QQGNVFSCSVMHEALHNHYTQKSLSLSPGK |
| SEQ ID NO: | DNA Heavy | CAAGTTCAGCTGCAAGAATCTGGCCCTGGCCTGGTCAAGCCTTCCGAGAC |
| 301 | Chain | ACTGTCTCTGACCTGCACCGTGTCTGGCTTCTCCCTGACCAATTACGGCGT |
| | | GCACTGGATCAGACAGCCTCCAGGCAAAGGCCTGGAATGGATCGGAGTG |
| | | ATTTGGAGAGGCGAGTCCACCGACTACAACGCCGCCTTCATGTCCAGAGT |
| | | GACCATCTCCAAGGACGACTCCAAGAGCCAGGTGTCCCTGAAGCTGTCCT |
| | | CTGTGACCGCTGCTGATACCGCCGTGTACTACTGTGCCAGAAACGCTGGCT |
| | | CCTCCGGCTGGTACTTTGATGTGTGGGGCCAGGGCACCACCGTGACAGTT |
| | | AGTTCTGCTAGCACCAAGGGCCCAAGTGTGTTTCCCCTGGCCCCCAGCAGC |
| | | AAGTCTACTTCCGGCGGAACTGCTGCCCTGGGTTGCCTGGTGAAGGACTA |
| | | CTTCCCCTGTCCCGTGACAGTGTCCTGGAACTCTGGGGCTCTGACTTCCGG |
| | | CGTGCACACCTTCCCCGCCGTGCTGCAGAGCAGCGGCCTGTACAGCCTGA |
| | | GCAGCGTGGTGACAGTGCCCTCCAGCTCTCTGGGAACCCAGACCTATATCT |
| | | GCAACGTGAACCACAAGCCCAGCAACACCAAGGTGGACAAGAGAGTGGA |
| | | GCCCAAGAGCTGCGACAAGACCCACACCTGCCCCCCCTGCCCAGCTCCAG |
| | | AACTGCTGGGAGGGCCTTCCGTGTTCCTGTTCCCCCCCAAGCCCAAGGACA |
| | | CCCTGATGATCAGCAGGACCCCCGAGGTGACCTGCGTGGTGGTGGCCGTG |
| | | TCCCACGAGGACCCAGAGGTGAAGTTCAACTGGTACGTGGACGGCGTGG |
| | | AGGTGCACAACGCCAAGACCAAGCCCAGAGAGGAGCAGTACAACAGCAC |
| | | CTACAGGGTGGTGTCCGTGCTGACCGTGCTGCACCAGGACTGGCTGAACG |
| | | GCAAAGAATACAAGTGCAAAGTCTCCAACAAGGCCCTGGCTGCCCCAATC |
| | | GAAAAGACAATCAGCAAGGCCAAGGGCCAGCCACGGGAGCCCCAGGTGT |
| | | ACACCCTGCCCCCCAGCCGGGAGGAGATGACCAAGAACCAGGTGTCCCTG |
| | | ACCTGTCTGGTGAAGGGCTTCTACCCCTGTGATATCGCCGTGGAGTGGGA |
| | | GAGCAACGGCCAGCCCGAGAACAACTACAAGACCACCCCCCCAGTGCTGG |
| | | ACAGCGACGGCAGCTTCTTCCTGTACAGCAAGCTGACCGTGGACAAGTCC |
| | | AGGTGGCAGCAGGGCAACGTGTTCAGCTGCAGCGTGATGCACGAGGCCC |
| | | TGCACAACCACTACACCCAGAAGTCCCTGAGCCTGAGCCCCGGCAAG |
| SEQ ID NO: | LCDR1 | KASQDVTSAVA |
| 277 | (Combined) | |
| SEQ ID NO: | LCDR2 | WTSTRHT |
| 278 | (Combined) | |
| SEQ ID NO: | LCDR3 | QQHYTTPLT |
| 279 | (Combined) | |
| SEQ ID NO: | LCDR1 | KASQDVTSAVA |
| 277 | (Kabat) | |
| SEQ ID NO: | LCDR2 | WTSTRHT |
| 278 | (Kabat) | |
| SEQ ID NO: | LCDR3 | QQHYTTPLT |
| 279 | (Kabat) | |
| SEQ ID NO: | LCDR1 | SQDVTSA |
| 280 | (Chothia) | |
| SEQ ID NO: | LCDR2 | WTS |
| 281 | (Chothia) | |
| SEQ ID NO: | LCDR3 | HYTTPL |
| 282 | (Chothia) | |
| SEQ ID NO: | LCDR1 | QDVTSA |
| 283 | (IMGT) | |
| SEQ ID NO: | LCDR2 | WTS |
| 281 | (IMGT) | |
| SEQ ID NO: | LCDR3 | QQHYTTPLT |
| 279 | (IMGT) | |
| SEQ ID NO: | VL | DIQLTQSPSFLSASVGDRVTITCKASQDVTSAVAWYQQKPGKAPKLLIYWTST |
| 284 | | RHTGVPSRFSGSGSGTEYTLTISSLQPEDFATYYCQQHYTTPLTFGQGTKLEIK |
| SEQ ID NO: | DNA VL | GATATTCAGCTGACCCAGTCTCCTAGCTTCCTGTCCGCTTCTGTGGGCGAC |
| 285 | | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAG |
| SEQ ID NO: | Light | DIQLTQSPSFLSASVGDRVTITCKASQDVTSAVAWYQQKPGKAPKLLIYWTST |
| 286 | Chain | RHTGVPSRFSGSGSGTEYTLTISSLQPEDFATYYCQQHYTTPLTFGQGTKLEIKR |
| | | TVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| SEQ ID NO: | DNA Light | GATATTCAGCTGACCCAGTCTCCTAGCTTCCTGTCCGCTTCTGTGGGCGAC |
| 287 | Chain | AGAGTGACCATCACATGCAAGGCCTCTCAGGACGTGACCTCTGCCGTGGC |
| | | TTGGTATCAGCAGAAGCCTGGCAAGGCCCCTAAGCTGCTGATCTACTGGA |
| | | CCTCCACCAGACACACCGGCGTGCCCTCTAGATTTTCCGGCTCTGGCTCTG |
| | | GCACCGAGTATACCCTGACAATCTCCAGCCTGCAGCCTGAGGACTTCGCCA |
| | | CCTACTACTGCCAGCAGCACTACACCACACCTCTGACCTTTGGCCAGGGCA |
| | | CCAAGCTGGAAATCAAGCGTACGGTGGCCGCTCCCAGCGTGTTCATCTTCC |
| | | CCCCCAGCGACGAGCAGCTGAAGAGTGGCACCGCCAGCGTGGTGTGCCT |
| | | GCTGAACAACTTCTACCCCCGGGAGGCCAAGGTGCAGTGGAAGGTGGAC |
| | | AACGCCCTGCAGAGCGGCAACAGCCAGGAGAGCGTCACCGAGCAGGACA |
| | | GCAAGGACTCCACCTACAGCCTGAGCAGCACCCTGACCCTGAGCAAGGCC |
| | | GACTACGAGAAGCATAAGGTGTACGCCTGCGAGGTGACCCACCAGGGCC |
| | | TGTCCAGCCCCGTGACCAAGAGCTTCAACAGGGGCGAGTGC |

Other anti-DC-SIGN antibodies or antibody fragments (e.g., antigen binding fragments) disclosed herein include amino acids that have been mutated, yet have at least 80, 85, 90, 95, 96, 97, 98, or 99 percent identity in the CDR regions with the CDR regions depicted in the sequences described in Table 8. In some embodiments, it includes mutant amino acid sequences wherein no more than 1, 2, 3, 4 or 5 amino acids have been mutated in the CDR regions when compared with the CDR regions depicted in the sequence described in Table 8.

Also provided herein are nucleic acid sequences that encode VH, VL, full length heavy chain, and full length light chain of antibodies and antigen binding fragments thereof that specifically bind to DC-SIGN, e.g., the nucleic acid sequences in Table 8. Such nucleic acid sequences can be optimized for expression in mammalian cells.

Other anti-DC-SIGN antibodies disclosed herein include those where the amino acids or nucleic acids encoding the amino acids have been mutated, yet have at least 80, 85, 90 95, 96, 97, 98, or 99 percent identity to the sequences described in Table 8. In some embodiments, antibodies or antigen binding fragments thereof include mutant amino acid sequences wherein no more than 1, 2, 3, 4 or 5 amino acids have been mutated in the variable regions when compared with the variable regions depicted in the sequence described in Table 8, while retaining substantially the same therapeutic activity.

Since each provided antibody binds to DC-SIGN, the VH, VL, full length light chain, and full length heavy chain sequences (amino acid sequences and the nucleotide sequences encoding the amino acid sequences) can be “mixed and matched” to create other DC-SIGN-binding antibodies disclosed herein. Such “mixed and matched” DC-SIGN-binding antibodies can be tested using binding assays known in the art (e.g., ELISAs, assays described in the Exemplification). When chains are mixed and matched, a VH sequence from a particular VH/VL pairing should be replaced with a structurally similar VH sequence. A full length heavy chain sequence from a particular full length heavy chain/full length light chain pairing should be replaced with a structurally similar full length heavy chain sequence. A VL sequence from a particular VH/VL pairing should be replaced with a structurally similar VL sequence. A full length light chain sequence from a particular full length heavy chain/full length light chain pairing should be replaced with a structurally similar full length light chain sequence.

Accordingly, in one embodiment, the invention provides an isolated monoclonal antibody or antigen binding region thereof having: a heavy chain variable region comprising an amino acid sequence of SEQ ID NO: 10; and a light chain variable region comprising an amino acid sequence of SEQ ID NO: 21; wherein the antibody specifically binds to DC-SIGN. In one embodiment, the invention provides an isolated monoclonal antibody or antigen binding region thereof having: a heavy chain variable region comprising an amino acid sequence of SEQ ID NO: 34; and a light chain variable region comprising an amino acid sequence of SEQ ID NO: 45; wherein the antibody specifically binds to DC-SIGN. In one embodiment, the invention provides an isolated monoclonal antibody or antigen binding region thereof having: a heavy chain variable region comprising an amino acid sequence of SEQ ID NO: 55; and a light chain variable region comprising an amino acid sequence of SEQ ID NO: 64; wherein the antibody specifically binds to DC-SIGN. In another embodiment, the invention provides (i) an isolated monoclonal antibody having: a full length heavy chain comprising an amino acid sequence of any of SEQ ID NOs: 12, 36 or 57; and a full length light chain comprising an amino acid sequence of any of SEQ ID NOs: 23, 47 or 66; or (ii) a functional protein comprising an antigen binding portion thereof.

In another embodiment, the present disclosure provides DC-SIGN-binding antibodies that comprise the heavy chain CDR1, CDR2 and CDR3 and light chain CDR1, CDR2 and CDR3 as described in Table 8, or combinations thereof. The amino acid sequences of the VH CDR1s of the antibodies are shown in SEQ ID NOs: 1, 4, 5, 7, 25, 28, 29 and 31. The amino acid sequences of the VH CDR2s of the antibodies and are shown in SEQ ID NOs: 2, 6, 8, 26, 30 and 32. The amino acid sequences of the VH CDR3s of the antibodies are shown in SEQ ID NO: 3, 9, 27 and 33. The amino acid sequences of the VL CDR1s of the antibodies are shown in SEQ ID NOs: 14, 17, 20, 38, 41 and 44. The amino acid sequences of the VL CDR2s of the antibodies are shown in SEQ ID Nos: 15, 18, 39 and 42. The amino acid sequences of the VL CDR3s of the antibodies are shown in SEQ ID NOs: 16, 19, 40 and 43.

Given that each of the antibodies binds DC-SIGN and that antigen-binding specificity is provided primarily by the CDR1, CDR2 and CDR3 regions, the VH CDR1, CDR2 and CDR3 sequences and VL CDR1, CDR2 and CDR3 sequences can be “mixed and matched” (i.e., CDRs from different antibodies can be mixed and match, although each antibody must contain a VH CDR1, CDR2 and CDR3 and a VL CDR1, CDR2 and CDR3 to create other DC-SIGN-binding binding molecules disclosed herein. Such “mixed and matched” DC-SIGN-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from CDR sequences shown herein for monoclonal antibodies of the present disclosure.

Accordingly, the present disclosure provides an isolated monoclonal antibody or antigen binding region thereof comprising a heavy chain CDR1 comprising an amino acid sequence selected from the group consisting of SEQ ID NOs: 1, 25, 49, 74, 88, 111, 138, 153, 178, 203, 227, 244, and 264; a heavy chain CDR2 comprising an amino acid sequence selected from the group consisting of SEQ ID NOs: 2, 26, 139, 154, 179, 204, 228, and 265; a heavy chain CDR3 comprising an amino acid sequence of SEQ ID NO: 3, 27, 50, 140, 155, 180, 205, 229, 245, and 266; a light chain CDR1 comprising an amino acid sequence selected from the group consisting of SEQ ID NOs: 14, 38, 59, 94, 166, 191, 216, 253, and 277; a light chain CDR2 comprising an amino acid sequence selected from the group consisting of SEQ ID NOs: 15, 39, 95, 167, 192, 217, 254, and 278; and a light chain CDR3 comprising an amino acid sequence selected from the group consisting of SEQ ID NOs: 16, 40, 60, 68, 82, 118, 124, 168, 193, 218, 238, 255, and 279; wherein the antibody specifically binds DC-SIGN.

In certain embodiments, an antibody that specifically binds to DC-SIGN is an antibody or antibody fragment (e.g., antigen binding fragment) that is described in Table 8.

In some embodiments, the antibody that specifically binds to human DC-SIGN comprises a heavy chain complementary determining region 1 (HCDR1) comprising the amino acid sequence of SEQ ID NO: 1; a heavy chain complementary determining region 2 (HCDR2) comprising the amino acid sequence of SEQ ID NO: 2; a heavy chain complementary determining region 3 (HCDR3) comprising the amino acid sequence of SEQ ID NO: 3; a light chain complementary determining region 1 (LCDR1) comprising the amino acid sequence of SEQ ID NO: 14; a light chain complementary determining region 2 (LCDR2) comprising the amino acid sequence of SEQ ID NO: 15; and a light chain complementary determining region 3 (LCDR3) comprising the amino acid sequence of SEQ ID NO: 16.

In some embodiments, the antibody that specifically binds to human DC-SIGN comprises a HCDR1 comprising the amino acid sequence of SEQ ID NO: 4; a HCDR2 comprising the amino acid sequence of SEQ ID NO: 2; a HCDR3 comprising the amino acid sequence of SEQ ID NO: 3; a LCDR1 comprising the amino acid sequence of SEQ ID NO: 14; a LCDR2 comprising the amino acid sequence of SEQ ID NO: 15; and a LCDR3 comprising the amino acid sequence of SEQ ID NO: 16.

In some embodiments, the antibody that specifically binds to human DC-SIGN comprises a HCDR1 comprising the amino acid sequence of SEQ ID NO: 5; a HCDR2 comprising the amino acid sequence of SEQ ID NO: 6; a HCDR3 comprising the amino acid sequence of SEQ ID NO: 3; a LCDR1 comprising the amino acid sequence of SEQ ID NO: 17; a LCDR2 comprising the amino acid sequence of SEQ ID NO: 18; and a LCDR3 comprising the amino acid sequence of SEQ ID NO: 19.

In some embodiments, the antibody that specifically binds to human DC-SIGN comprises a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; a LCDR1 comprising the amino acid sequence of SEQ ID NO: 20; a LCDR2 comprising the amino acid sequence of SEQ ID NO: 18; and a LCDR3 comprising the amino acid sequence of SEQ ID NO: 16.

In some embodiments, the antibody that specifically binds to human DC-SIGN comprises a HCDR1 comprising the amino acid sequence of SEQ ID NO: 25; a HCDR2 comprising the amino acid sequence of SEQ ID NO: 26; a HCDR3 comprising the amino acid sequence of SEQ ID NO: 27; a LCDR1 comprising the amino acid sequence of SEQ ID NO: 38; a LCDR2 comprising the amino acid sequence of SEQ ID NO: 39; and a LCDR3 comprising the amino acid sequence of SEQ ID NO: 40.

In some embodiments, the antibody that specifically binds to human DC-SIGN comprises a HCDR1 comprising the amino acid sequence of SEQ ID NO: 28; a HCDR2 comprising the amino acid sequence of SEQ ID NO: 26; a HCDR3 comprising the amino acid sequence of SEQ ID NO: 27; a LCDR1 comprising the amino acid sequence of SEQ ID NO: 38; a LCDR2 comprising the amino acid sequence of SEQ ID NO: 39; and a LCDR3 comprising the amino acid sequence of SEQ ID NO: 40.

In some embodiments, the antibody that specifically binds to human DC-SIGN comprises a HCDR1 comprising the amino acid sequence of SEQ ID NO: 29; a HCDR2 comprising the amino acid sequence of SEQ ID NO: 30; a HCDR3 comprising the amino acid sequence of SEQ ID NO: 27; a LCDR1 comprising the amino acid sequence of SEQ ID NO: 41; a LCDR2 comprising the amino acid sequence of SEQ ID NO: 42; and a LCDR3 comprising the amino acid sequence of SEQ ID NO: 43.

In some embodiments, the antibody that specifically binds to human DC-SIGN comprises a HCDR1 comprising the amino acid sequence of SEQ ID NO: 31; a HCDR2 comprising the amino acid sequence of SEQ ID NO: 32; a HCDR3 comprising the amino acid sequence of SEQ ID NO: 33; a LCDR1 comprising the amino acid sequence of SEQ ID NO: 44; a LCDR2 comprising the amino acid sequence of SEQ ID NO: 42; and a LCDR3 comprising the amino acid sequence of SEQ ID NO: 40.

In some embodiments, the antibody that specifically binds to human DC-SIGN comprises a HCDR1 comprising the amino acid sequence of SEQ ID NO: 49; a HCDR2 comprising the amino acid sequence of SEQ ID NO: 26; a HCDR3 comprising the amino acid sequence of SEQ ID NO: 50; a LCDR1 comprising the amino acid sequence of SEQ ID NO: 59; a LCDR2 comprising the amino acid sequence of SEQ ID NO: 39; and a LCDR3 comprising the amino acid sequence of SEQ ID NO: 60.

In some embodiments, the antibody that specifically binds to human DC-SIGN comprises a HCDR1 comprising the amino acid sequence of SEQ ID NO: 51; a HCDR2 comprising the amino acid sequence of SEQ ID NO: 26; a HCDR3 comprising the amino acid sequence of SEQ ID NO: 50; a LCDR1 comprising the amino acid sequence of SEQ ID NO: 59; a LCDR2 comprising the amino acid sequence of SEQ ID NO: 39; and a LCDR3 comprising the amino acid sequence of SEQ ID NO: 60.

In some embodiments, the antibody that specifically binds to human DC-SIGN comprises a HCDR1 comprising the amino acid sequence of SEQ ID NO: 52; a HCDR2 comprising the amino acid sequence of SEQ ID NO: 30; a HCDR3 comprising the amino acid sequence of SEQ ID NO: 50; a LCDR1 comprising the amino acid sequence of SEQ ID NO: 61; a LCDR2 comprising the amino acid sequence of SEQ ID NO: 42; and a LCDR3 comprising the amino acid sequence of SEQ ID NO: 62.

In some embodiments, the antibody that specifically binds to human DC-SIGN comprises a HCDR1 comprising the amino acid sequence of SEQ ID NO: 53; a HCDR2 comprising the amino acid sequence of SEQ ID NO: 32; a HCDR3 comprising the amino acid sequence of SEQ ID NO: 54; a LCDR1 comprising the amino acid sequence of SEQ ID NO: 63; a LCDR2 comprising the amino acid sequence of SEQ ID NO: 42; and a LCDR3 comprising the amino acid sequence of SEQ ID NO: 60.

In some embodiments, the antibody that specifically binds to human DC-SIGN comprises a heavy chain variable region comprising the amino acid sequence of SEQ ID NO: 10, and a light chain variable region comprising the amino acid sequence of SEQ ID NO: 21.

In some embodiments, the antibody that specifically binds to human DC-SIGN comprises a heavy chain comprising the amino acid sequence of SEQ ID NO: 34, and a light chain comprising the amino acid sequence of SEQ ID NO: 45.

In some embodiments, the antibody that specifically binds to human DC-SIGN comprises a heavy chain comprising the amino acid sequence of SEQ ID NO: 55, and a light chain comprising the amino acid sequence of SEQ ID NO: 64.

In some embodiments, the antibody that specifically binds to human DC-SIGN comprises a heavy chain comprising the amino acid sequence of SEQ ID NO: 34, and a light chain comprising the amino acid sequence of SEQ ID NO: 70.

In some embodiments, the antibody that specifically binds to human DC-SIGN comprises a heavy chain comprising the amino acid sequence of SEQ ID NO: 78, and a light chain comprising the amino acid sequence of SEQ ID NO: 84.

In some embodiments, the antibody that specifically binds to human DC-SIGN comprises a heavy chain comprising the amino acid sequence of SEQ ID NO: 90, and a light chain comprising the amino acid sequence of SEQ ID NO: 99.

In some embodiments, the antibody that specifically binds to human DC-SIGN comprises a heavy chain comprising the amino acid sequence of SEQ ID NO: 103, and a light chain comprising the amino acid sequence of SEQ ID NO: 107.

In some embodiments, the antibody that specifically binds to human DC-SIGN comprises a heavy chain comprising the amino acid sequence of SEQ ID NO: 114, and a light chain comprising the amino acid sequence of SEQ ID NO: 120.

In some embodiments, the antibody that specifically binds to human DC-SIGN comprises a heavy chain comprising the amino acid sequence of SEQ ID NO: 55, and a light chain comprising the amino acid sequence of SEQ ID NO: 126.

In some embodiments, the antibody that specifically binds to human DC-SIGN comprises a heavy chain comprising the amino acid sequence of SEQ ID NO: 78, and a light chain comprising the amino acid sequence of SEQ ID NO: 130.

In some embodiments, the antibody that specifically binds to human DC-SIGN comprises a heavy chain comprising the amino acid sequence of SEQ ID NO: 90, and a light chain comprising the amino acid sequence of SEQ ID NO: 134.

In some embodiments, the antibody that specifically binds to human DC-SIGN comprises a heavy chain comprising the amino acid sequence of SEQ ID NO: 145, and a light chain comprising the amino acid sequence of SEQ ID NO: 149.

In some embodiments, the antibody that specifically binds to human DC-SIGN comprises a heavy chain comprising the amino acid sequence of SEQ ID NO: 162, and a light chain comprising the amino acid sequence of SEQ ID NO: 174.

In some embodiments, the antibody that specifically binds to human DC-SIGN comprises a heavy chain comprising the amino acid sequence of SEQ ID NO: 187, and a light chain comprising the amino acid sequence of SEQ ID NO: 199.

In some embodiments, the antibody that specifically binds to human DC-SIGN comprises a heavy chain comprising the amino acid sequence of SEQ ID NO: 212, and a light chain comprising the amino acid sequence of SEQ ID NO: 223.

In some embodiments, the antibody that specifically binds to human DC-SIGN comprises a heavy chain comprising the amino acid sequence of SEQ ID NO: 234, and a light chain comprising the amino acid sequence of SEQ ID NO: 240.

In some embodiments, the antibody that specifically binds to human DC-SIGN comprises a heavy chain comprising the amino acid sequence of SEQ ID NO: 249, and a light chain comprising the amino acid sequence of SEQ ID NO: 260.

In some embodiments, the antibody that specifically binds to human DC-SIGN comprises a heavy chain comprising the amino acid sequence of SEQ ID NO: 273, and a light chain comprising the amino acid sequence of SEQ ID NO: 284.

In some embodiments, the antibody that specifically binds to human DC-SIGN comprises a heavy chain comprising the amino acid sequence of SEQ ID NO: 288, and a light chain comprising the amino acid sequence of SEQ ID NO: 292.

In some embodiments, the antibody that specifically binds to human DC-SIGN comprises a heavy chain comprising the amino acid sequence of SEQ ID NO: 298, and a light chain comprising the amino acid sequence of SEQ ID NO: 284.

In some embodiments, the present disclosure provides antibodies or antibody fragments (e.g., antigen binding fragments) that specifically bind an epitope in human DC-SIGN. In some embodiments, the present disclosure provides antibodies or antibody fragments (e.g., antigen binding fragments) that specifically bind to an epitope in human DC-SIGN, wherein the epitope comprises amino acid sequence of SEQ ID NOs: 320-323.

In some embodiments, the present disclosure provides antibodies or antibody fragments (e.g., antigen binding fragments) that specifically bind to human DC-SIGN, but not human L-SIGN. For example, the present disclosure provides antibodies or antibody fragments (e.g., antigen binding fragments) that specifically bind to human DC-SIGN at an affinity that is at least 1×, at least 2×, at least 3×, at least 4×, at least 5×, at least 10×, at least 20×, at least 50×, at least 100×, at least 1,000× higher than its affinity to human L-SIGN.

Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 2003/48731. As will be appreciated by one of skill in the art, practically anything to which an antibody can specifically bind could be an epitope. An epitope can comprises those residues to which the antibody binds.

The present invention also provides anti-DC-SIGN antibodies or antigen binding fragments thereof that comprise modifications in the constant regions of the heavy chain, light chain, or both the heavy and light chain wherein particular amino acid residues have mutated to cysteines, also referred to herein at “CysMab” or “Cys” antibodies. As discussed herein, drug moieties may be conjugated site specifically and with control over the number of drug moieties (“DAR Controlled”) to cysteine residues on antibodies. Cysteine modifications to antibodies for the purposes of site specifically controlling immunoconjugation are disclosed, for example, in WO2014/124316, which is incorporated herein by reference in its entirety.

In some embodiments, the anti-DC-SIGN antibodies have been modified at positions 152 and/or 375 of the heavy chain, wherein the positions are defined according to the EU numbering system. Namely, the modifications are E152C and/or S375C. In some embodiments, the anti-DC-SIGN antibodies have been modified at position 152 of the heavy chain, wherein the positions are defined according to the EU numbering system. Namely, the modification is E152C. In some embodiments, the anti-DC-SIGN antibodies have been modified at position 375 of the heavy chain, wherein the positions are defined according to the EU numbering system. Namely, the modification is S375C. In other embodiments, the anti-DC-SIGN antibodies have been modified at position 360 of the heavy chain and position 107 of the kappa light chain, wherein the positions are defined according to the EU numbering system. Namely, the modifications are K360C and K107C.

The present invention also provides nucleic acid sequences that encode the VH, VL, the full length heavy chain, and the full length light chain of the antibodies that specifically bind to P-cadherin. Such nucleic acid sequences can be optimized for expression in mammalian cells.

Identification of Epitopes and Antibodies that Bind to the Same Epitope

The present invention also provides antibodies and antibody fragments (e.g., antigen binding fragments) that specifically bind to the same epitope as the anti-DC-SIGN antibodies described in Table 8, or cross compete with the antibodies described in Table 8. Additional antibodies and antibody fragments (e.g., antigen binding fragments) can therefore be identified based on their ability to cross-compete (e.g., to competitively inhibit the binding of, in a statistically significant manner) with other antibodies of the invention in DC-SIGN binding assays, for example, via BIACORE or assays known to persons skilled in the art for measuring binding. The ability of a test antibody to inhibit the binding of antibodies and antibody fragments (e.g., antigen binding fragments) of the present invention to a DC-SIGN (e.g., human DC-SIGN) demonstrates that the test antibody can compete with that antibody or antibody fragment (e.g., antigen binding fragments) for binding to DC-SIGN; such an antibody may, according to non-limiting theory, bind to the same or a related (e.g., a structurally similar or spatially proximal or overlapping) epitope on the DC-SIGN protein as the antibody or antibody fragment (e.g., antigen binding fragments) with which it competes. In certain embodiments, the antibodies that bind to the same epitope on DC-SIGN as the antibodies or antibody fragments (e.g., antigen binding fragments) described in Table 8 are human or humanized monoclonal antibodies. Such human or humanized monoclonal antibodies can be prepared and isolated as described herein.

### Modification of Framework or Fc Region

Antibodies and antibody conjugates disclosed herein may comprise modified antibodies or antigen binding fragments thereof that comprise modifications to framework residues within VH and/or VL, e.g. to improve the properties of the antibody/antibody conjugate.

In some embodiments, framework modifications are made to decrease immunogenicity of an antibody. For example, one approach is to “back-mutate” one or more framework residues to a corresponding germline sequence. Such residues can be identified by comparing antibody framework sequences to germline sequences from which the antibody is derived. To “match” framework region sequences to desired germline configuration, residues can be “back-mutated” to a corresponding germline sequence by, for example, site-directed mutagenesis. Such “back-mutated” antibodies are also intended to be encompassed by the invention.

Another type of framework modification involves mutating one or more residues within a framework region, or even within one or more CDR regions, to remove T-cell epitopes to thereby reduce potential immunogenicity of the antibody. This approach is also referred to as “deimmunization” and is described in further detail in U.S. Patent Publication No. 20030153043 by Carr et al.

In addition or alternative to modifications made within a framework or CDR regions, antibodies disclosed herein may be engineered to include modifications within the Fc region, typically to alter one or more functional properties of the antibody, such as serum half-life, complement fixation, Fc receptor binding, and/or antigen-dependent cellular cytotoxicity.

Furthermore, an antibody disclosed herein may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH₁ is modified such that the number of cysteine residues in the hinge region is altered, e.g., increased or decreased. This approach is described further in U.S. Pat. No. 5,677,425 by Bodmer et al. The number of cysteine residues in the hinge region of CH₁ is altered to, for example, facilitate assembly of the light and heavy chains or to increase or decrease the stability of the antibody.

In some embodiments antibodies or antibody fragments (e.g., antigen binding fragment) useful in antibody conjugates disclosed herein include modified or engineered antibodies, such as an antibody modified to introduce one or more cysteine residues as sites for conjugation to a drug moiety (Junutula J R, et al.: Nat Biotechnol 2008, 26:925-932). In one embodiment, the invention provides a modified antibody or antibody fragment thereof comprising a substitution of one or more amino acids with cysteine at the positions described herein. Sites for cysteine substitution are in the constant regions of the antibody and are thus applicable to a variety of antibodies, and the sites are selected to provide stable and homogeneous conjugates. A modified antibody or fragment can have two or more cysteine substitutions, and these substitutions can be used in combination with other antibody modification and conjugation methods as described herein. Methods for inserting cysteine at specific locations of an antibody are known in the art, see, e.g., Lyons et al, (1990) Protein Eng., 3:703-708, WO 2011/005481, WO2014/124316, WO 2015/138615. In certain embodiments a modified antibody or antibody fragment comprises a substitution of one or more amino acids with cysteine on its constant region selected from positions 117, 119, 121, 124, 139, 152, 153, 155, 157, 164, 169, 171, 174, 189, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody or antibody fragment, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain.

In particular embodiments a modified antibody or antibody fragment thereof comprises a combination of substitution of two amino acids with cysteine on its constant regions, wherein the modified antibody or antibody fragment thereof comprises cysteines at positions 152 and 375 of an antibody heavy chain, wherein the positions are numbered according to the EU system.

In other particular embodiments a modified antibody or antibody fragment thereof comprises a substitution of one amino acid with cysteine at position 360 of an antibody heavy chain and wherein the positions are numbered according to the EU system.

In other particular embodiments a modified antibody or antibody fragment thereof comprises a substitution of one amino acid with cysteine at position 107 of an antibody light chain and wherein the positions are numbered according to the EU system, and wherein the light chain is a kappa chain.

In additional embodiments antibodies or antibody fragments (e.g., antigen binding fragment) useful in antibody conjugates disclosed herein include modified or engineered antibodies, such as an antibody modified to introduce one or more other reactive amino acid (other than cysteine), including Pcl (pyrroline-carboxy-lysine), pyrrolysine, peptide tags (such as S6, A1 and ybbR tags), and non-natural amino acids, in place of at least one amino acid of the native sequence, thus providing a reactive site on the antibody or antigen binding fragment for conjugation to a drug moiety of Formula (I) or subformulae thereof. For example, the antibodies or antibody fragments can be modified to incorporate Pcl or pyrrolysine (W. Ou et al. (2011) PNAS 108 (26), 10437-10442; WO2014124258) or unnatural amino acids (J. Y. Axup, et al. Proc Natl Acad Sci USA, 109 (2012), pp. 16101-16106; for review, see C. C. Liu and P. G. Schultz (2010) Annu Rev Biochem 79, 413-444; C. H. Kim, et al., (2013) Curr Opin Chem Biol. 17, 412-419) as sites for conjugation to a drug. Similarly, peptide tags for enzymatic conjugation methods can be introduced into an antibody (Strop P. et al. Chem Biol. 2013, 20(2):161-7; Rabuka D., Curr Opin Chem Biol. 2010 December; 14(6):790-6; Rabuka D, et al., Nat Protoc. 2012, 7(6):1052-67). One other example is the use of 4′-phosphopantetheinyl transferases (PPTase) for the conjugation of Coenzyme A analogs (WO2013184514; Grinewald J, et al., Bioconjug Chem. 2015 Dec. 16; 26(12):2554-62). Methods for conjugating such modified or engineered antibodies with payloads or linker-payload combinations are known in the art.

In another embodiment, an Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl Protein A (SpA) binding relative to native Fc-hinge domain SpA binding. This approach is described in further detail in U.S. Pat. No. 6,165,745 by Ward et al.

In yet other embodiments, an Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another embodiment, one or more amino acids selected from amino acid residues can be replaced with a different amino acid residue such that the antibody has altered Clq binding and/or reduced or abolished complement dependent cytotoxicity (CDC). This approach is described in, e.g., U.S. Pat. No. 6,194,551 by Idusogie et al.

In another embodiment, one or more amino acid residues are altered to thereby alter the ability of the antibody to fix complement. This approach is described in, e.g., the PCT Publication WO 94/29351 by Bodmer et al. Allotypic amino acid residues include, but are not limited to, constant region of a heavy chain of the IgG1, IgG2, and IgG3 subclasses as well as constant region of a light chain of the kappa isotype as described by Jefferis et al., MAbs. 1:332-338 (2009).

In a further embodiment, the Fc region is modified to “silence” the effector function of the antibody, for example, reduce or eliminate the ability of the antibody to mediate antibody dependent cellular cytotoxicity (ADCC) and/or antibody dependent cellular phagocytosis (ADCP). This can be achieve, for example, by introducing a mutation in the Fc region of the antibodies. Such mutations have been described in the art: LALA and N297A (Strohl, W., 2009, Curr. Opin. Biotechnol. vol. 20(6):685-691); and D265A (Baudino et al., 2008, J. Immunol. 181: 6664-69; Strohl, W., supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the so-called DAPA mutant comprising D265A and P329A mutations in the IgG1 Fc amino acid sequence. Another silent IgG1 antibody comprises the N297A mutation, which results in aglycosylated/non-glycosylated antibodies.

In yet another embodiment, the Fc region is modified to increase the ability of the antibody to mediate antibody dependent cellular cytotoxicity (ADCC) and/or antibody dependent cellular phagocytosis (ADCP), for example, by modifying one or more amino acid residues to increase the affinity of the antibody for an activating Fcγ receptor, or to decrease the affinity of the antibody for an inhibitory Fcγ receptor. Human activating Fcγ receptors include FcγRIa, FcγRIIa, FcγRIIIa, and FcγRIIIb, and human inhibitory Fcγ receptor includes FcγRIIb. This approach is described in, e.g., the PCT Publication WO 00/42072 by Presta. Moreover, binding sites on human IgG1 for FcγRI, FcγRII, FcγRIII and FcRn have been mapped and variants with improved binding have been described (see Shields et al., J. Biol. Chem. 276:6591-6604, 2001). Optimization of Fc-mediated effector functions of monoclonal antibodies such as increased ADCC/ADCP function has been described (see Strohl, W. R., Current Opinion in Biotechnology 2009; 20:685-691.) In some embodiments, an antibody conjugate comprises an immunoglobulin heavy chain comprising a mutation or combination of mutations conferring enhanced ADCC/ADCP function, e.g., one or more mutations selected from G236A, S239D, F243L, P2471, D280H, K290S, R292P, S298A, S298D, S298V, Y300L, V3051, A330L, 1332E, E333A, K334A, A339D, A339Q, A339T, P396L (all positions by EU numbering).

In another embodiment, the Fc region is modified to increase the ability of the antibody to mediate ADCC and/or ADCP, for example, by modifying one or more amino acids to increase the affinity fo the antibody for an activating receptor that would typically not recognize the parent antibody, such as FcαRI. This approach is descried in, e.g., Borrok et al., mAbs. 7(4):743-751. In particular embodiments, an antibody conjugate comprises an immunoglobulin heavy chain comprising a mutation or a fusion of one or more antibody sequences conferring enhanced ADCC and/or ADCP function.

In still another embodiment, glycosylation of an antibody is modified. For example, an aglycosylated antibody can be made (i.e., the antibody lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in, e.g., U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

### Production of Anti-DC-SIGN Antibodies

Anti-DC-SIGN antibodies and antibody fragments (e.g., antigen binding fragments) thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

Also provided herein are polynucleotides encoding antibodies described herein, e.g., polynucleotides encoding heavy or light chain variable regions or segments comprising complementarity determining regions as described herein. In some embodiments, a polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide of SEQ ID NO: 11, 35, 56, 79, 91, 104, 115, 146, 163, 188, 213, 235, 250, 274, 289, or 299. In some embodiments, a polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide of SEQ ID NO: 22, 46, 65, 71, 85, 100, 108, 121, 127, 131, 135, 150, 175, 200, 224, 241, 261, 285, or 293.

In some embodiments, a polynucleotide encoding the heavy chain has at least 85%, 89%, 90%, 91%, 92%, 93%, 94%, 95%, 96%, 97%, 98%, 99%, or 100% nucleic acid sequence identity with a polynucleotide of any of SEQ ID NOs: 13, 37, 58, 81, 93, 106, 117, 148, 165, 190, 215, 237, 252, 276, 291, or 301. In some embodiments, a polynucleotide encoding the light chain has at least 85%, 89%, 90%, 91%, 92%, 93%, 94%, 95%, 96%, 97%, 98%, 99%, or 100% nucleic acid sequence identity with a polynucleotide of SEQ ID NO: 24, 48, 67, 73, 87, 102, 110, 123, 129, 133, 137, 152, 177, 202, 226, 243, 263, 287, or 295.

Some polynucleotides disclosed herein encode a variable region of an anti-DC-SIGN antibody. Some polynucleotides disclosed herein encode both a variable region and a constant region of an anti-DC-SIGN antibody. Some polynucleotide sequences encode a polypeptide that comprises variable regions of both a heavy chain and a light chain of an anti-DC-SIGN antibody. Some polynucleotides encode two polypeptide segments that respectively are substantially identical to the variable regions of a heavy chain and a light chain of any anti-DC-SIGN antibodies disclosed herein.

Polynucleotide sequences can be produced by de novo solid-phase DNA synthesis or by PCR mutagenesis of an existing sequence encoding an antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided are expression vectors and host cells for producing antibodies described herein. Various expression vectors can be employed to express polynucleotides encoding antibody chains or binding fragments. Both viral-based and nonviral expression vectors can be used to produce antibodies in a mammalian host cell.

Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of polynucleotides and polypeptides in mammalian (e.g., human) cells include pThioHis A, B & C, pCDNATM3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

Choice of expression vector depends on the intended host cells in which a vector is to be expressed. Typically, expression vectors contain a promoter and other regulatory sequences (e.g., enhancers) that are operably linked to polynucleotides encoding an antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by host cells. In addition to promoters, other regulatory elements may also be required or desired for efficient expression of an antibody chain or fragment. Elements typically include an ATG initiation codon and adjacent ribosome binding site or other sequences. In addition, efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, an SV40 enhancer or CMV enhancer may be used to increase expression in mammalian host cells.

Expression vectors may also provide a secretion signal sequence position to form a fusion protein with polypeptides encoded by inserted antibody sequences. More often, inserted antibody sequences are linked to a signal sequence before inclusion in the vector. Vectors to be used to receive sequences encoding antibody light and heavy chain variable domains sometimes also encode constant regions or parts thereof. Such vectors allow expression of variable regions as fusion proteins with constant regions, thereby leading to production of intact antibodies or fragments thereof. Typically, such constant regions are human.

Host cells for harboring and expressing antibody chains can be either prokaryotic or eukaryotic. E. coli is one prokaryotic host useful for cloning and expressing polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as a lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express polypeptides, including antibodies. Insect cells in combination with baculovirus vectors can also be used.

In some particular embodiments, mammalian host cells are used to express and produce polypeptides of the present disclosure. For example, they can be either a hybridoma cell line expressing endogenous immunoglobulin genes (e.g., myeloma hybridoma clones) or a mammalian cell line harboring an exogenous expression vector (e.g., the SP2/0 myeloma cells). These include any normal mortal or normal or abnormal immortal animal or human cell. For example, a number of suitable host cell lines capable of secreting intact immunoglobulins have been developed, including various CHO cell lines, Cos cell lines, HeLa cells, myeloma cell lines, transformed B-cells and hybridomas. Use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. Expression vectors usually contain promoters derived from mammalian genes or from mammalian viruses. Suitable promoters may be constitutive, cell type-specific, stage-specific, and/or modulatable or regulatable. Useful promoters include, but are not limited to, a metallothionein promoter, a constitutive adenovirus major late promoter, a dexamethasoneinducible MMTV promoter, a SV40 promoter, a MRP polIII promoter, a constitutive MPSV promoter, a tetracycline-inducible CMV promoter (such as the human immediate-early CMV promoter), a constitutive CMV promoter, and promoter-enhancer combinations known in the art.

Methods for introducing expression vectors containing polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express antibody chains or binding fragments can be prepared using expression vectors disclosed herein which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Therapeutic Uses and Methods of Treatment

Provided antibody conjugates are useful in a variety of applications including, but not limited to, treatment of cancer. In certain embodiments, antibody conjugates provided herein are useful for inhibiting tumor growth, reducing tumor volume, inducing differentiation, and/or reducing the tumorigenicity of a tumor. The methods of use can be in vitro, ex vivo, or in vivo methods.

In some embodiments, provided herein are methods of treating, preventing, or ameliorating a disease, e.g., a cancer, in a subject in need thereof, e.g., a human patient, by administering to the subject any of the antibody conjugates described herein. Also provided is use of the antibody conjugates of the invention to treat or prevent disease in a subject, e.g., a human patient. Additionally provided is use of antibody conjugates in treatment or prevention of disease in a subject. In some embodiments provided are antibody conjugates for use in manufacture of a medicament for treatment or prevention of disease in a subject. In certain embodiments, the disease treated with antibody conjugates is a cancer.

In one aspect, the immunoconjugates described herein can be used to treat a solid tumor. Examples of solid tumors include malignancies, e.g., sarcomas, adenocarcinomas, blastomas, and carcinomas, of the various organ systems, such as those affecting liver, lung, breast, lymphoid, biliarintestinal (e.g., colon), genitourinary tract (e.g., renal, urothelial cells), prostate and pharynx. Adenocarcinomas include malignancies such as most colon cancers, rectal cancer, renal-cell carcinoma, liver cancer, small cell lung cancer, non-small cell carcinoma of the lung, cancer of the small intestine and cancer of the esophagus. In one embodiment, the cancer is a melanoma, e.g., an advanced stage melanoma. Examples of other cancers that can be treated include bone cancer, pancreatic cancer, skin cancer, cancer of the head or neck, cutaneous or intraocular malignant melanoma, uterine cancer, ovarian cancer, rectal cancer, colorectal cancer, cancer of the anal region, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, environmentally induced cancers including those induced by asbestos, and combinations of said cancers.

In another aspect, the immunoconjugates described herein can be used to treat a hematological cancer. Hematological cancers include leukemia, lymphoma, and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

In some embodiments, the cancer is a hematologic cancer including but is not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with a tumor antigen expression includes, but not limited to, e.g., atypical and/or non-classical cancers, malignancies, precancerous conditions or proliferative diseases expressing a tumor antigen as described herein. Metastatic lesions of the aforementioned cancers can also be treated or prevented using the methods and compositions of the invention.

Method of administration of such antibody conjugates include, but are not limited to, parenteral (e.g., intravenous) administration, e.g., injection as a bolus or continuous infusion over a period of time, oral administration, intramuscular administration, intratumoral administration, intramuscular administration, intraperitoneal administration, intracerobrospinal administration, subcutaneous administration, intra-articular administration, intrasynovial administration, injection to lymph nodes, or intrathecal administration.

For treatment of disease, appropriate dosage of antibody conjugates of the present invention depends on various factors, such as the type of disease to be treated, the severity and course of the disease, the responsiveness of the disease, previous therapy, patient's clinical history, and so on. Antibody conjugates can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of a particular antibody conjugate. In some embodiments, dosage is from 0.01 mg to 20 mg (e.g., 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg) per kg of body weight, and can be given once or more daily, weekly, monthly or yearly. In certain embodiments, the antibody conjugate of the present invention is given once every two weeks or once every three weeks. In certain embodiments, the antibody conjugate of the present invention is given only once. The treating physician can estimate repetition rates for dosing based on measured residence times and concentrations of the drug in bodily fluids or tissues.

### Combination Therapy

In certain instances, an antibody conjugate of the present invention can be combined with other therapeutic agents, such as other anti-cancer agents, anti-allergic agents, anti-nausea agents (or anti-emetics), pain relievers, cytoprotective agents, and combinations thereof.

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), epirubicin (Ellence®), oxaliplatin (Eloxatin®), exemestane (Aromasin®), letrozole (Femara®), and fulvestrant (Faslodex®).

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In one embodiment, the present invention provides a method of treating cancer by administering to a subject in need thereof antibody conjugate of the present invention in combination with one or more anti-HER2 antibodies, e.g., trastuzumab, pertuzumab, margetuximab, or HT-19 described above, or with other anti-HER2 conjugates, e.g., ado-trastuzumab emtansine (also known as Kadcyla®, or T-DM1).

In one embodiment, the present invention provides a method of treating cancer by administering to a subject in need thereof antibody conjugate of the present invention in combination with one or more tyrosine kinase inhibitors, including but not limited to, EGFR inhibitors, Her3 inhibitors, IGFR inhibitors, and Met inhibitors.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (Gilotrif®); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3β,5β,6α)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI 166, CAS187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R3; MDX0447 (CAS 339151-96-1); and ch806 (mAb-806, CAS 946414-09-1).

Other HER2 inhibitors include but are not limited to, Neratinib (HKI-272, (2E)-N-[4-[[3-chloro-4-[(pyridin-2-yl) methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3a□,5□,6a□)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU 11271); (3Z)-N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU 11274); (3Z)-N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU 11606); 6-[Difluoro[6-(1-methyl-1H-pyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin 3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGFR inhibitors include but are not limited to, BMS-754807, XL-228, OSI-906, GSK0904529A, A-928605, AXL1717, KW-2450, MK0646, AMG479, IMCA12, MEDI-573, and B1836845. See e.g., Yee, JNCI, 104; 975 (2012) for review.

In another embodiment, the present invention provides a method of treating cancer by administering to a subject in need thereof antibody conjugate of the present invention in combination with one or more proliferation signaling pathway inhibitors, including but not limited to, MEK inhibitors, BRAF inhibitors, PI3K/Akt inhibitors, SHP2 inhibitors, and also mTOR inhibitors, and CDK inhibitors.

For example, mitogen-activated protein kinase (MEK) inhibitors include but are not limited to, XL-518 (also known as GDC-0973, Cas No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

BRAF inhibitors include, but are not limited to, Vemurafenib (or Zelboraf®), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®), or Ipilimumab (or MDX-010, MDX-101, or Yervoy).

Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC0941, RG7321, GNE0941, Pictrelisib, or Pictilisib; and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethylcyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); (S)-N1-(4-methyl-5-(2-(1,1,1-trifluoro-2-methylpropan-2-yl)pyridin-4-yl)thiazol-2-yl)pyrrolidine-1,2-dicarboxamide (also known as BYL719 or Alpelisib); 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide (also known as GDC0032, RG7604, or Taselisib).

mTOR inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9] hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-□-aspartylL-serine-(SEQ ID NO: 932), inner salt (SF1126, CAS 936487-67-1).

CDK inhibitors include but are not limited to, Palbociclib (also known as PD-0332991, Ibrance®, 6-Acetyl-8-cyclopentyl-5-methyl-2-{[5-(1-piperazinyl)-2-pyridinyl]amino}pyrido[2,3-d]pyrimidin-7(8H)-one).

In yet another embodiment, the present invention provides a method of treating cancer by administering to a subject in need thereof antibody conjugate of the present invention in combination with one or more pro-apoptotics, including but not limited to, IAP inhibitors, BCL2 inhibitors, MCL1 inhibitors, TRAIL agents, CHK inhibitors.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679, all of which are incorporated herein by reference.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinylpyrazolo[1,5-a]pyrimidin-7-amine (SCH900776, CAS 891494-63-6); 5-(3-Fluorophenyl)-3-ureidothiophene-2-carboxylic acid N-[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kI]pyrrolo[3,4-i][1,6]benzodiazocine-1,3(2H)-dione (SB-218078, CAS 135897-06-2); and TAT-S216A (YGRKKRRQRRRLYRSPAMPENL (SEQ ID NO: 929)), and CBP501 ((d-Bpa)sws(d-Phe-F5)(d-Cha)rrrqrr).

In a further embodiment, the present invention provides a method of treating cancer by administering to a subject in need thereof antibody conjugate of the present invention in combination with one or more immunomodulators (e.g., one or more of an activator of a costimulatory molecule or an inhibitor of an immune checkpoint molecule).

In certain embodiments, the immunomodulator is an activator of a costimulatory molecule. In one embodiment, the agonist of the costimulatory molecule is selected from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

### GITR Aqonists

In certain embodiments, the agonist of the costimulatory molecule is a GITR agonist. In some embodiments, the GITR agonist is GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Aqonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of MAB7 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-GITR antibody molecule comprises a heavy chain variable region (VH) comprising a VHCDR1 amino acid sequence of SEQ ID NO: 909, a VHCDR2 amino acid sequence of SEQ ID NO: 911, and a VHCDR3 amino acid sequence of SEQ ID NO: 913; and a light chain variable region (VL) comprising a VLCDR1 amino acid sequence of SEQ ID NO: 914, a VLCDR2 amino acid sequence of SEQ ID NO: 916, and a VLCDR3 amino acid sequence of SEQ ID NO: 918, each disclosed in Table 9.

In one embodiment, the anti-GITR antibody molecule comprises a VH comprising the amino acid sequence of SEQ ID NO: 901, or an amino acid sequence at least 85%, 90%, 95%, or 99% identical or higher to SEQ ID NO: 901. In one embodiment, the anti-GITR antibody molecule comprises a VL comprising the amino acid sequence of SEQ ID NO: 902, or an amino acid sequence at least 85%, 90%, 95%, or 99% identical or higher to SEQ ID NO: 902. In one embodiment, the anti-GITR antibody molecule comprises a VH comprising the amino acid sequence of SEQ ID NO: 901 and a VL comprising the amino acid sequence of SEQ ID NO: 902.

In one embodiment, the antibody molecule comprises a VH encoded by the nucleotide sequence of SEQ ID NO: 905, or a nucleotide sequence at least 85%, 90%, 95%, or 99% identical or higher to SEQ ID NO: 905. In one embodiment, the antibody molecule comprises a VL encoded by the nucleotide sequence of SEQ ID NO: 906, or a nucleotide sequence at least 85%, 90%, 95%, or 99% identical or higher to SEQ ID NO: 906. In one embodiment, the antibody molecule comprises a VH encoded by the nucleotide sequence of SEQ ID NO: 905 and a VL encoded by the nucleotide sequence of SEQ ID NO: 906.

In one embodiment, the anti-GITR antibody molecule comprises a heavy chain comprising the amino acid sequence of SEQ ID NO: 903, or an amino acid sequence at least 85%, 90%, 95%, or 99% identical or higher to SEQ ID NO: 903. In one embodiment, the anti-GITR antibody molecule comprises a light chain comprising the amino acid sequence of SEQ ID NO: 904, or an amino acid sequence at least 85%, 90%, 95%, or 99% identical or higher to SEQ ID NO: 904. In one embodiment, the anti-GITR antibody molecule comprises a heavy chain comprising the amino acid sequence of SEQ ID NO: 903 and a light chain comprising the amino acid sequence of SEQ ID NO: 904.

In one embodiment, the antibody molecule comprises a heavy chain encoded by the nucleotide sequence of SEQ ID NO: 907, or a nucleotide sequence at least 85%, 90%, 95%, or 99% identical or higher to SEQ ID NO: 907. In one embodiment, the antibody molecule comprises a light chain encoded by the nucleotide sequence of SEQ ID NO: 908, or a nucleotide sequence at least 85%, 90%, 95%, or 99% identical or higher to SEQ ID NO: 908. In one embodiment, the antibody molecule comprises a heavy chain encoded by the nucleotide sequence of SEQ ID NO: 907 and a light chain encoded by the nucleotide sequence of SEQ ID NO: 908.

The antibody molecules described herein can be made by vectors, host cells, and methods described in WO 2016/057846, incorporated by reference in its entirety.

TABLE 9
Amino acid and nucleotide sequences of exemplary anti-GITR antibody molecule

MAB7

| | | |
|---|---|---|
| SEQ ID NO: 901 | VH | EVQLVESGGGLVQSGGSLRLSCAASGFSLSSYGVDW |
| | | VRQAPGKGLEWVGVIWGGGGTYYASSLMGRFTISRD |
| | | NSKNTLYLQMNSLRAEDTAVYYCARHAYGHDGGFAM |
| | | DYWGQGTLVTVSS |
| SEQ ID NO: 902 | VL | EIVMTQSPATLSVSPGERATLSCRASESVSSNVAWYQ |
| | | QRPGQAPRLLIYGASNRATGIPARFSGSGSGTDFTLTI |
| | | SRLEPEDFAVYYCGQSYSYPFTFGQGTKLEIK |
| SEQ ID NO: 903 | Heavy | EVQLVESGGGLVQSGGSLRLSCAASGFSLSSYGVDW |
| | Chain | VRQAPGKGLEWVGVIWGGGGTYYASSLMGRFTISRD |
| | | NSKNTLYLQMNSLRAEDTAVYYCARHAYGHDGGFAM |
| | | DYWGQGTLVTVSSASTKGPSVFPLAPSSKSTSGGTA |
| | | ALGCLVKDYFPEPVTVSWNSGALTSGVHTFPAVLQSS |
| | | GLYSLSSVVTVPSSSLGTQTYICNVNHKPSNTKVDKR |
| | | VEPKSCDKTHTCPPCPAPELLGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCKV |
| | | SNKALPAPIEKTISKAKGQPREPQVYTLPPSREEMTKN |
| | | QVSLTCLVKGFYPSDIAVEWESNGQPENNYKTTPPVL |
| | | DSDGSFFLYSKLTVDKSRWQQGNVFSCSVMHEALHN |
| | | HYTQKSLSLSPGK |
| SEQ ID NO: 904 | Light | EIVMTQSPATLSVSPGERATLSCRASESVSSNVAWYQ |
| | Chain | QRPGQAPRLLIYGASNRATGIPARFSGSGSGTDFTLTI |
| | | SRLEPEDFAVYYCGQSYSYPFTFGQGTKLEIKRTVAA |
| | | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| SEQ ID NO: 905 | DNA | GAGGTGCAGCTGGTGGAATCTGGCGGCGGACTGG |
| | VH | TGCAGTCCGGCGGCTCTCTGAGACTGTCTTGCGCT |
| | | GCCTCCGGCTTCTCCCTGTCCTCTTACGGCGTGGA |
| | | CTGGGTGCGACAGGCCCCTGGCAAGGGCCTGGAA |
| | | TGGGTGGGAGTGATCTGGGGCGGAGGCGGCACCT |
| | | ACTACGCCTCTTCCCTGATGGGCCGGTTCACCATCT |
| | | CCCGGGACAACTCCAAGAACACCCTGTACCTGCAG |
| | | ATGAACTCCCTGCGGGCCGAGGACACCGCCGTGTA |
| | | CTACTGCGCCAGACACGCCTACGGCCACGACGGC |
| | | GGCTTCGCCATGGATTATTGGGGCCAGGGCACCCT |
| | | GGTGACAGTGTCCTCC |
| SEQ ID NO: 906 | DNA | GAGATCGTGATGACCCAGTCCCCCGCCACCCTGTC |
| | VL | TGTGTCTCCCGGCGAGAGAGCCACCCTGAGCTGCA |
| | | GAGCCTCCGAGTCCGTGTCCTCCAACGTGGCCTGG |
| | | TATCAGCAGAGACCTGGTCAGGCCCCTCGGCTGCT |
| | | GATCTACGGCGCCTCTAACCGGGCCACCGGCATCC |
| | | CTGCCAGATTCTCCGGCTCCGGCAGCGGCACCGAC |
| | | TTCACCCTGACCATCTCCCGGCTGGAACCCGAGGA |
| | | CTTCGCCGTGTACTACTGCGGCCAGTCCTACTCATA |
| | | CCCCTTCACCTTCGGCCAGGGCACCAAGCTGGAAA |
| | | TCAAG |
| SEQ ID NO: 907 | DNA | GAGGTGCAGCTGGTGGAATCTGGCGGCGGACTGG |
| | Heavy | TGCAGTCCGGCGGCTCTCTGAGACTGTCTTGCGCT |
| | Chain | GCCTCCGGCTTCTCCCTGTCCTCTTACGGCGTGGA |
| | | CTGGGTGCGACAGGCCCCTGGCAAGGGCCTGGAA |
| | | TGGGTGGGAGTGATCTGGGGCGGAGGCGGCACCT |
| | | ACTACGCCTCTTCCCTGATGGGCCGGTTCACCATCT |
| | | CCCGGGACAACTCCAAGAACACCCTGTACCTGCAG |
| | | ATGAACTCCCTGCGGGCCGAGGACACCGCCGTGTA |
| | | CTACTGCGCCAGACACGCCTACGGCCACGACGGC |
| | | GGCTTCGCCATGGATTATTGGGGCCAGGGCACCCT |
| | | GGTGACAGTGTCCTCCGCTAGCACCAAGGGCCCAA |
| | | GTGTGTTTCCCCTGGCCCCCAGCAGCAAGTCTACTT |
| | | CCGGCGGAACTGCTGCCCTGGGTTGCCTGGTGAAG |
| | | GACTACTTCCCCGAGCCCGTGACAGTGTCCTGGAA |
| | | CTCTGGGGCTCTGACTTCCGGCGTGCACACCTTCC |
| | | CCGCCGTGCTGCAGAGCAGCGGCCTGTACAGCCT |
| | | GAGCAGCGTGGTGACAGTGCCCTCCAGCTCTCTGG |
| | | GAACCCAGACCTATATCTGCAACGTGAACCACAAGC |
| | | CCAGCAACACCAAGGTGGACAAGAGAGTGGAGCCC |
| | | AAGAGCTGCGACAAGACCCACACCTGCCCCCCCTG |
| | | CCCAGCTCCAGAACTGCTGGGAGGGCCTTCCGTGT |
| | | TCCTGTTCCCCCCCAAGCCCAAGGACACCCTGATG |
| | | ATCAGCAGGACCCCCGAGGTGACCTGCGTGGTGGT |
| | | GGACGTGTCCCACGAGGACCCAGAGGTGAAGTTCA |
| | | ACTGGTACGTGGACGGCGTGGAGGTGCACAACGC |
| | | CAAGACCAAGCCCAGAGAGGAGCAGTACAACAGCA |
| | | CCTACAGGGTGGTGTCCGTGCTGACCGTGCTGCAC |
| | | CAGGACTGGCTGAACGGCAAAGAATACAAGTGCAA |
| | | AGTCTCCAACAAGGCCCTGCCAGCCCCAATCGAAA |
| | | AGACAATCAGCAAGGCCAAGGGCCAGCCACGGGA |
| | | GCCCCAGGTGTACACCCTGCCCCCCAGCCGGGAG |
| | | GAGATGACCAAGAACCAGGTGTCCCTGACCTGTCT |
| | | GGTGAAGGGCTTCTACCCCAGCGATATCGCCGTGG |
| | | AGTGGGAGAGCAACGGCCAGCCCGAGAACAACTAC |
| | | AAGACCACCCCCCCAGTGCTGGACAGCGACGGCA |
| | | GCTTCTTCCTGTACAGCAAGCTGACCGTGGACAAGT |
| | | CCAGGTGGCAGCAGGGCAACGTGTTCAGCTGCAGC |
| | | GTGATGCACGAGGCCCTGCACAACCACTACACCCA |
| | | GAAGTCCCTGAGCCTGAGCCCCGGCAAG |
| SEQ ID NO: 908 | DNA | GAGATCGTGATGACCCAGTCCCCCGCCACCCTGTC |
| | Light | TGTGTCTCCCGGCGAGAGAGCCACCCTGAGCTGCA |
| | Chain | GAGCCTCCGAGTCCGTGTCCTCCAACGTGGCCTGG |
| | | TATCAGCAGAGACCTGGTCAGGCCCCTCGGCTGCT |
| | | GATCTACGGCGCCTCTAACCGGGCCACCGGCATCC |
| | | CTGCCAGATTCTCCGGCTCCGGCAGCGGCACCGAC |
| | | TTCACCCTGACCATCTCCCGGCTGGAACCCGAGGA |
| | | CTTCGCCGTGTACTACTGCGGCCAGTCCTACTCATA |
| | | CCCCTTCACCTTCGGCCAGGGCACCAAGCTGGAAA |
| | | TCAAGCGTACGGTGGCCGCTCCCAGCGTGTTCATC |
| | | TTCCCCCCCAGCGACGAGCAGCTGAAGAGCGGCAC |
| | | CGCCAGCGTGGTGTGCCTGCTGAACAACTTCTACC |
| | | CCCGGGAGGCCAAGGTGCAGTGGAAGGTGGACAA |
| | | CGCCCTGCAGAGCGGCAACAGCCAGGAGAGCGTC |
| | | ACCGAGCAGGACAGCAAGGACTCCACCTACAGCCT |
| | | GAGCAGCACCCTGACCCTGAGCAAGGCCGACTACG |
| | | AGAAGCATAAGGTGTACGCCTGCGAGGTGACCCAC |
| | | CAGGGCCTGTCCAGCCCCGTGACCAAGAGCTTCAA |
| | | CAGGGGCGAGTGC |
| SEQ ID NO: 909 | HCDR1 | SYGVD |
| KABAT) | | |
| SEQ ID NO: 910 | HCDR1 | GFSLSSY |
| (CHOTHIA) | | |
| SEQ ID NO: 911 | HCDR2 | VIWGGGGTYYASSLMG |
| (KABAT) | | |
| SEQ ID NO: 912 | HCDR2 | WGGGG |
| (CHOTHIA) | | |
| SEQ ID NO: 913 | HCDR3 | HAYGHDGGFAMDY |
| (KABAT) | | |
| SEQ ID NO: 913 | HCDR3 | HAYGHDGGFAMDY |
| (CHOTHIA) | | |
| SEQ ID NO: 914 | LCDR1 | RASESVSSNVA |
| (KABAT) | | |
| SEQ ID NO: 915 | LCDR1 | SESVSSN |
| (CHOTHIA) | | |
| SEQ ID NO: 916 | LCDR2 | GASNRAT |
| (KABAT) | | |
| SEQ ID NO: 917 | LCDR2 | GAS |
| (CHOTHIA) | | |
| SEQ ID NO: 918 | LCDR3 | GQSYSYPFT |
| (KABAT) | | |
| SEQ ID NO: 919 | LCDR3 | SYSYPF |
| (CHOTHIA) | | |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 10.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:596, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 10
Amino acid sequence of other exemplary
anti-GITR antibody molecules

BMS-986156

| | | |
|---|---|---|
| SEQ ID NO: 920 | VH | QVQLVESGGGVVQPGRSLRLSCAASGFTFSSYGMH |
| | | WVRQAPGKGLEWVAVIWYEGSNKYYADSVKGRFTI |
| | | SRDNSKNTLYLQMNSLRAEDTAVYYCARGGSMVRG |
| | | DYYYGMDVWGQGTTVTVSS |
| SEQ ID NO: 921 | VL | AIQLTQSPSSLSASVGDRVTITCRASQGISSALAW |
| | | YQQKPGKAPKLLIYDASSLESGVPSRFSGSGSGTD |
| | | FTLTISSLQPEDFATYYCQQFNSYPYTFGQGTKLE |
| | | IK |

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFRbeta. In one embodiment, the inhibitor of an immune checkpoint molecule inhibits PD-1, PD-L1, LAG-3, TIM-3 or CTLA4, or any combination thereof. The term “inhibition” or “inhibitor” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40%, 50% or more is included by this term. Thus, inhibition need not be 100%.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) selected from, e.g., the heavy chain constant regions of IgG1, IgG2, IgG3, IgG4, IgM, IgA1, IgA2, IgD, and IgE; particularly, selected from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specificity, e.g., a second binding specificity to TIM-3, LAG-3, or PD-L2. In one embodiment, the bispecific antibody molecule binds to PD-1 or PD-L1 and TIM-3. In another embodiment, the bispecific antibody molecule binds to PD-1 or PD-L1 and LAG-3. In another embodiment, the bispecific antibody molecule binds to PD-1 and PD-L1. In yet another embodiment, the bispecific antibody molecule binds to PD-1 and PD-L2. In another embodiment, the bispecific antibody molecule binds to TIM-3 and LAG-3. Any combination of the aforesaid molecules can be made in a multispecific antibody molecule, e.g., a trispecific antibody that includes a first binding specificity to PD-1 or PD-1, and a second and third binding specifities to two or more of: TIM-3, LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3 or CTLA4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule.

Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present invention. Any of the antibody molecules known in the art or disclosed herein can be used in the aforesaid combinations of inhibitors of checkpoint molecule.

### PD-1 Inhibitors

In some embodiments, the antibody conjugate of the present invention is administered in combination with a PD-1 inhibitor. In some embodiments, the PD-1 inhibitor is selected from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 11 (e.g., from the heavy and light chain variable region sequences of BAP049-Clone-E or BAP049-Clone-B disclosed in Table 11), or encoded by a nucleotide sequence shown in Table 11. In some embodiments, the CDRs are according to the Kabat definition (e.g., as set out in Table 11). In some embodiments, the CDRs are according to the Chothia definition (e.g., as set out in Table 11). In some embodiments, the CDRs are according to the combined CDR definitions of both Kabat and Chothia (e.g., as set out in Table 11). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 11, or encoded by a nucleotide sequence shown in Table 11.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 11.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 11.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 11
Amino acid and nucleotide sequences of exemplary anti-PD-1 antibody molecules
BAP049-Clone-B HC

| | | |
|---|---|---|
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQ |
| | | ATGQGLEWMGNIYPGTGGSNFDEKFKNRVTITADKSTSTA |
| | | YMELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAA |
| | VH | GCCCGGCGAGTCACTGAGAATTAGCTGTAAAGGTTCAG |
| | | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQ |
| | chain | ATGQGLEWMGNIYPGTGGSNFDEKFKNRVTITADKSTSTA |
| | | YMELSSLRSEDTAVYYCTRWTTGTGAYWGQGTTVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 509 | DNA | GAGGTGCAGCTGGTGCAGTCAGGCGCCGAAGTGAAGAA |
| | heavy | GCCCGGCGAGTCACTGAGAATTAGCTGTAAAGGTTCAG |
| | chain | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGCGCTAGCACTAAGGGCCCGTCCGTGTTCCCCCT |
| | | GGCACCTTGTAGCCGGAGCACTAGCGAATCCACCGCTG |
| | | CCCTCGGCTGCCTGGTCAAGGATTACTTCCCGGAGCCC |
| | | GTGACCGTGTCCTGGAACAGCGGAGCCCTGACCTCCGG |
| | | AGTGCACACCTTCCCCGCTGTGCTGCAGAGCTCCGGGC |
| | | TGTACTCGCTGTCGTCGGTGGTCACGGTGCCTTCATCTA |
| | | GCCTGGGTACCAAGACCTACACTTGCAACGTGGACCAC |
| | | AAGCCTTCCAACACTAAGGTGGACAAGCGCGTCGAATC |
| | | GAAGTACGGCCCACCGTGCCCGCCTTGTCCCGCGCCG |
| | | GAGTTCCTCGGCGGTCCCTCGGTCTTTCTGTTCCCACC |
| | | GAAGCCCAAGGACACTTTGATGATTTCCCGCACCCCTGA |
| | | AGTGACATGCGTGGTCGTGGACGTGTCACAGGAAGATC |
| | | CGGAGGTGCAGTTCAATTGGTACGTGGATGGCGTCGAG |
| | | GTGCACAACGCCAAAACCAAGCCGAGGGAGGAGCAGTT |
| | | CAACTCCACTTACCGCGTCGTGTCCGTGCTGACGGTGC |
| | | TGCATCAGGACTGGCTGAACGGGAAGGAGTACAAGTGC |
| | | AAAGTGTCCAACAAGGGACTTCCTAGCTCAATCGAAAAG |
| | | ACCATCTCGAAAGCCAAGGGACAGCCCCGGGAACCCCA |
| | | AGTGTATACCCTGCCACCGAGCCAGGAAGAAATGACTAA |
| | | GAACCAAGTCTCATTGACTTGCCTTGTGAAGGGCTTCTA |
| | | CCCATCGGATATCGCCGTGGAATGGGAGTCCAACGGCC |
| | | AGCCGGAAAACAACTACAAGACCACCCCTCCGGTGCTG |
| | | GACTCAGACGGATCCTTCTTCCTCTACTCGCGGCTGACC |
| | | GTGGATAAGAGCAGATGGCAGGAGGGAAATGTGTTCAG |
| | | CTGTTCTGTGATGCATGAAGCCCTGCACAACCACTACAC |
| | | TCAGAAGTCCCTGTCCCTCTCCCTGGGA |

BAP049-Clone-B LC

| | | |
|---|---|---|
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTW |
| | | YQQKPGKAPKLLIYWASTRESGVPSRFSGSGSGTDFTFTIS |
| | | SLQPEDIATYYCQNDYSYPYTFGQGTKVEIK |
| SEQ ID NO: 517 | DNA | GAGATCGTCCTGACTCAGTCACCCGCTACCCTGAGCCT |
| | VL | GAGCCCTGGCGAGCGGGCTACACTGAGCTGTAAATCTA |
| | | GTCAGTCACTGCTGGATAGCGGTAATCAGAAGAACTTCC |
| | | TGACCTGGTATCAGCAGAAGCCCGGTAAAGCCCCTAAG |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGCAGCCCGAGGATATCGC |
| | | TACCTACTACTGTCAGAACGACTATAGCTACCCCTACAC |
| | | CTTCGGTCAAGGCACTAAGGTCGAGATTAAG |
| SEQ ID NO: 518 | Light | EIVLTQSPATLSLSPGERATLSCKSSQSLLDSGNQKNFLTW |
| | chain | YQQKPGKAPKLLIYWASTRESGVPSRFSGSGSGTDFTFTIS |
| | | SLQPEDIATYYCQNDYSYPYTFGQGTKVEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| SEQ ID NO: 519 | DNA | GAGATCGTCCTGACTCAGTCACCCGCTACCCTGAGCCT |
| | light | GAGCCCTGGCGAGCGGGCTACACTGAGCTGTAAATCTA |
| | chain | GTCAGTCACTGCTGGATAGCGGTAATCAGAAGAACTTCC |
| | | TGACCTGGTATCAGCAGAAGCCCGGTAAAGCCCCTAAG |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGCAGCCCGAGGATATCGC |
| | | TACCTACTACTGTCAGAACGACTATAGCTACCCCTACAC |
| | | CTTCGGTCAAGGCACTAAGGTCGAGATTAAGCGTACGG |
| | | TGGCCGCTCCCAGCGTGTTCATCTTCCCCCCCAGCGAC |
| | | GAGCAGCTGAAGAGCGGCACCGCCAGCGTGGTGTGCC |
| | | TGCTGAACAACTTCTACCCCCGGGAGGCCAAGGTGCAG |
| | | TGGAAGGTGGACAACGCCCTGCAGAGCGGCAACAGCCA |
| | | GGAGAGCGTCACCGAGCAGGACAGCAAGGACTCCACCT |
| | | ACAGCCTGAGCAGCACCCTGACCCTGAGCAAGGCCGAC |
| | | TACGAGAAGCATAAGGTGTACGCCTGCGAGGTGACCCA |
| | | CCAGGGCCTGTCCAGCCCCGTGACCAAGAGCTTCAACA |
| | | GGGGCGAGTGC |

BAP049-Clone-E HC

| | | |
|---|---|---|
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQ |
| | | ATGQGLEWMGNIYPGTGGSNFDEKFKNRVTITADKSTSTA |
| | | YMELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAA |
| | VH | GCCCGGCGAGTCACTGAGAATTAGCTGTAAAGGTTCAG |
| | | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQ |
| | chain | ATGQGLEWMGNIYPGTGGSNFDEKFKNRVTITADKSTSTA |
| | | YMELSSLRSEDTAVYYCTRWTTGTGAYWGQGTTVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 509 | DNA | GAGGTGCAGCTGGTGCAGTCAGGCGCCGAAGTGAAGAA |
| | heavy | GCCCGGCGAGTCACTGAGAATTAGCTGTAAAGGTTCAG |
| | chain | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGCGCTAGCACTAAGGGCCCGTCCGTGTTCCCCCT |
| | | GGCACCTTGTAGCCGGAGCACTAGCGAATCCACCGCTG |
| | | CCCTCGGCTGCCTGGTCAAGGATTACTTCCCGGAGCCC |
| | | GTGACCGTGTCCTGGAACAGCGGAGCCCTGACCTCCGG |
| | | AGTGCACACCTTCCCCGCTGTGCTGCAGAGCTCCGGGC |
| | | TGTACTCGCTGTCGTCGGTGGTCACGGTGCCTTCATCTA |
| | | GCCTGGGTACCAAGACCTACACTTGCAACGTGGACCAC |
| | | AAGCCTTCCAACACTAAGGTGGACAAGCGCGTCGAATC |
| | | GAAGTACGGCCCACCGTGCCCGCCTTGTCCCGCGCCG |
| | | GAGTTCCTCGGCGGTCCCTCGGTCTTTCTGTTCCCACC |
| | | GAAGCCCAAGGACACTTTGATGATTTCCCGCACCCCTGA |
| | | AGTGACATGCGTGGTCGTGGACGTGTCACAGGAAGATC |
| | | CGGAGGTGCAGTTCAATTGGTACGTGGATGGCGTCGAG |
| | | GTGCACAACGCCAAAACCAAGCCGAGGGAGGAGCAGTT |
| | | CAACTCCACTTACCGCGTCGTGTCCGTGCTGACGGTGC |
| | | TGCATCAGGACTGGCTGAACGGGAAGGAGTACAAGTGC |
| | | AAAGTGTCCAACAAGGGACTTCCTAGCTCAATCGAAAAG |
| | | ACCATCTCGAAAGCCAAGGGACAGCCCCGGGAACCCCA |
| | | AGTGTATACCCTGCCACCGAGCCAGGAAGAAATGACTAA |
| | | GAACCAAGTCTCATTGACTTGCCTTGTGAAGGGCTTCTA |
| | | CCCATCGGATATCGCCGTGGAATGGGAGTCCAACGGCC |
| | | AGCCGGAAAACAACTACAAGACCACCCCTCCGGTGCTG |
| | | GACTCAGACGGATCCTTCTTCCTCTACTCGCGGCTGACC |
| | | GTGGATAAGAGCAGATGGCAGGAGGGAAATGTGTTCAG |
| | | CTGTTCTGTGATGCATGAAGCCCTGCACAACCACTACAC |
| | | TCAGAAGTCCCTGTCCCTCTCCCTGGGA |

BAP049-Clone-E LC

| | | |
|---|---|---|
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTW |
| | | YQQKPGQAPRLLIYWASTRESGVPSRFSGSGSGTDFTFTI |
| | | SSLEAEDAATYYCQNDYSYPYTFGQGTKVEIK |
| SEQ ID NO: 521 | DNA | GAGATCGTCCTGACTCAGTCACCCGCTACCCTGAGCCT |
| | VL | GAGCCCTGGCGAGCGGGCTACACTGAGCTGTAAATCTA |
| | | GTCAGTCACTGCTGGATAGCGGTAATCAGAAGAACTTCC |
| | | TGACCTGGTATCAGCAGAAGCCCGGTCAAGCCCCTAGA |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGGAAGCCGAGGACGCCG |
| | | CTACCTACTACTGTCAGAACGACTATAGCTACCCCTACA |
| | | CCTTCGGTCAAGGCACTAAGGTCGAGATTAAG |
| SEQ ID NO: 522 | Light | EIVLTQSPATLSLSPGERATLSCKSSQSLLDSGNQKNFLTW |
| | chain | YQQKPGQAPRLLIYWASTRESGVPSRFSGSGSGTDFTFTI |
| | | SSLEAEDAATYYCQNDYSYPYTFGQGTKVEIKRTVAAPSVF |
| | | IFPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| SEQ ID NO: 523 | DNA | GAGATCGTCCTGACTCAGTCACCCGCTACCCTGAGCCT |
| | light | GAGCCCTGGCGAGCGGGCTACACTGAGCTGTAAATCTA |
| | chain | GTCAGTCACTGCTGGATAGCGGTAATCAGAAGAACTTCC |
| | | TGACCTGGTATCAGCAGAAGCCCGGTCAAGCCCCTAGA |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGGAAGCCGAGGACGCCG |
| | | CTACCTACTACTGTCAGAACGACTATAGCTACCCCTACA |
| | | CCTTCGGTCAAGGCACTAAGGTCGAGATTAAGCGTACG |
| | | GTGGCCGCTCCCAGCGTGTTCATCTTCCCCCCCAGCGA |
| | | CGAGCAGCTGAAGAGCGGCACCGCCAGCGTGGTGTGC |
| | | CTGCTGAACAACTTCTACCCCCGGGAGGCCAAGGTGCA |
| | | GTGGAAGGTGGACAACGCCCTGCAGAGCGGCAACAGC |
| | | CAGGAGAGCGTCACCGAGCAGGACAGCAAGGACTCCAC |
| | | CTACAGCCTGAGCAGCACCCTGACCCTGAGCAAGGCCG |
| | | ACTACGAGAAGCATAAGGTGTACGCCTGCGAGGTGACC |
| | | CACCAGGGCCTGTCCAGCCCCGTGACCAAGAGCTTCAA |
| | | CAGGGGCGAGTGC |

BAP049-Clone-B HC

| | | |
|---|---|---|
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGA |
| (Kabat) | , | GAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGA |
| (Kabat) | | GAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

### Other Exemplary PD-1 Inhibitors

selected from In some embodiments, the anti-PD-1 antibody is Nivolumab (CAS Registry Number: 946414-94-4). Alternative names for Nivolumab include MDX-1106, MDX-1106-04, ONO-4538, BMS-936558 or OPDIVO®. Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and PCT Publication No. WO2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 12.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, PCT Publication No. WO2009/114335, and U.S. Pat. No. 8,354,509, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 12.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in PCT Publication No. WO2009/101611, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 12.

Other anti-PD1 antibodies are disclosed in U.S. Pat. No. 8,609,089, US Publication No. 2010028330, and/or US Publication No. 20120114649, incorporated by reference in their entirety. Other anti-PD1 antibodies include AMP 514 (Amplimmune).

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224 (B7-DCIg (Amplimmune), e.g., disclosed in WO 2010/027827 and WO 2011/066342, incorporated by reference in their entirety).

TABLE 12
Amino acid sequences of other exemplary anti-PD-1 antibody molecules
Nivolumab

| | | |
|---|---|---|
| SEQ ID NO: 535 | Heavy | QVQLVESGGGVVQPGRSLRLDCKASGITFSNSGMHWVRQAPG |
| | chain | KGLEWVAVIWYDGSKRYYADSVKGRFTISRDNSKNTLFLQMNSL |
| | | RAEDTAVYYCATNDDYWGQGTLVTVSSASTKGPSVFPLAPCSR |
| | | STSESTAALGCLVKDYFPEPVTVSWNSGALTSGVHTFPAVLQSS |
| | | GLYSLSSVVTVPSSSLGTKTYTCNVDHKPSNTKVDKRVESKYGP |
| | | PCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQ |
| | | DWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLS |
| | | LSLGK |
| SEQ ID NO: 536 | Light | EIVLTQSPATLSLSPGERATLSCRASQSVSSYLAWYQQKPGQAP |
| | chain | RLLIYDASNRATGIPARFSGSGSGTDFTLTISSLEPEDFAVYYCQ |
| | | QSSNWPRTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |

Pembrolizumab

| | | |
|---|---|---|
| SEQ ID NO: 537 | Heavy | QVQLVQSGVEVKKPGASVKVSCKASGYTFTNYYMYWVRQAPG |
| | chain | QGLEWMGGINPSNGGTNFNEKFKNRVTLTTDSSTTTAYMELKSL |
| | | QFDDTAVYYCARRDYRFDMGFDYWGQGTTVTVSSASTKGPSV |
| | | FPLAPCSRSTSESTAALGCLVKDYFPEPVTVSWNSGALTSGVHT |
| | | FPAVLQSSGLYSLSSVVTVPSSSLGTKTYTCNVDHKPSNTKVDK |
| | | RVESKYGPPCPPCPAPEFLGGPSVFLFPPKPKDTLMISRTPEVT |
| | | CVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPEN |
| | | NYKTTPPVLDSDGSFFLYSRLTVDKSRWQEGNVFSCSVMHEAL |
| | | HNHYTQKSLSLSLGK |
| SEQ ID NO: 538 | Light | EIVLTQSPATLSLSPGERATLSCRASKGVSTSGYSYLHWYQQKP |
| | chain | GQAPRLLIYLASYLESGVPARFSGSGSGTDFTLTISSLEPEDFAV |
| | | YYCQHSRDLPLTFGGGTKVEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |

Pidilizumab

| | | |
|---|---|---|
| SEQ ID NO: 539 | Heavy | QVQLVQSGSELKKPGASVKISCKASGYTFTNYGMNWVRQAPGQ |
| | chain | GLQWMGWINTDSGESTYAEEFKGRFVFSLDTSVNTAYLQITSLT |
| | | AEDTGMYFCVRVGYDALDYWGQGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKRVEPK |
| | | SCDKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| SEQ ID NO: 540 | Light | EIVLTQSPSSLSASVGDRVTITCSARSSVSYMHWFQQKPGKAPK |
| | chain | LWIYRTSNLASGVPSRFSGSGSGTSYCLTINSLQPEDFATYYCQ |
| | | QRSSFPLTFGGGTKLEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |

### PD-L1 Inhibitors

In certain embodiments, the inhibitor of an immune checkpoint molecule is an inhibitor of PD-L1. In some embodiments, the antibody conjugate of the present invention is administered in combination with a PD-L1 inhibitor. In some embodiments, the PD-L1 inhibitor is selected from FAZ053 (Novartis), Atezolizumab (Genentech/Roche), Avelumab (Merck Serono and Pfizer), Durvalumab (Medlmmune/AstraZeneca), or BMS-936559 (Bristol-Myers Squibb).

### Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 13 (e.g., from the heavy and light chain variable region sequences of BAP058-Clone O or BAP058-Clone N disclosed in Table 13), or encoded by a nucleotide sequence shown in Table 13. In some embodiments, the CDRs are according to the Kabat definition (e.g., as set out in Table 13). In some embodiments, the CDRs are according to the Chothia definition (e.g., as set out in Table 13). In some embodiments, the CDRs are according to the combined CDR definitions of both Kabat and Chothia (e.g., as set out in Table 13). In one embodiment, the combination of Kabat and Chothia CDR of VH CDR1 comprises the amino acid sequence GYTFTSYWMY (SEQ ID NO: 647). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 13, or encoded by a nucleotide sequence shown in Table 13.

In one embodiment, the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611, each disclosed in Table 13.

In one embodiment, the anti-PD-L1 antibody molecule comprises a VH comprising a VHCDR1 encoded by the nucleotide sequence of SEQ ID NO: 628, a VHCDR2 encoded by the nucleotide sequence of SEQ ID NO: 629, and a VHCDR3 encoded by the nucleotide sequence of SEQ ID NO: 630; and a VL comprising a VLCDR1 encoded by the nucleotide sequence of SEQ ID NO: 633, a VLCDR2 encoded by the nucleotide sequence of SEQ ID NO: 634, and a VLCDR3 encoded by the nucleotide sequence of SEQ ID NO: 635, each disclosed in Table 13.

In one embodiment, the anti-PD-L1 antibody molecule comprises a VH comprising the amino acid sequence of SEQ ID NO: 606, or an amino acid sequence at least 85%, 90%, 95%, or 99% identical or higher to SEQ ID NO: 606. In one embodiment, the anti-PD-L1 antibody molecule comprises a VL comprising the amino acid sequence of SEQ ID NO: 616, or an amino acid sequence at least 85%, 90%, 95%, or 99% identical or higher to SEQ ID NO: 616. In one embodiment, the anti-PD-L1 antibody molecule comprises a VH comprising the amino acid sequence of SEQ ID NO: 620, or an amino acid sequence at least 85%, 90%, 95%, or 99% identical or higher to SEQ ID NO: 620. In one embodiment, the anti-PD-L1 antibody molecule comprises a VL comprising the amino acid sequence of SEQ ID NO: 624, or an amino acid sequence at least 85%, 90%, 95%, or 99% identical or higher to SEQ ID NO: 624. In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

In one embodiment, the antibody molecule comprises a VH encoded by the nucleotide sequence of SEQ ID NO: 607, or a nucleotide sequence at least 85%, 90%, 95%, or 99% identical or higher to SEQ ID NO: 607. In one embodiment, the antibody molecule comprises a VL encoded by the nucleotide sequence of SEQ ID NO: 617, or a nucleotide sequence at least 85%, 90%, 95%, or 99% identical or higher to SEQ ID NO: 617. In one embodiment, the antibody molecule comprises a VH encoded by the nucleotide sequence of SEQ ID NO: 621, or a nucleotide sequence at least 85%, 90%, 95%, or 99% identical or higher to SEQ ID NO: 621.

In one embodiment, the antibody molecule comprises a VL encoded by the nucleotide sequence of SEQ ID NO: 625, or a nucleotide sequence at least 85%, 90%, 95%, or 99% identical or higher to SEQ ID NO: 625. In one embodiment, the antibody molecule comprises a VH encoded by the nucleotide sequence of SEQ ID NO: 607 and a VL encoded by the nucleotide sequence of SEQ ID NO: 617. In one embodiment, the antibody molecule comprises a VH encoded by the nucleotide sequence of SEQ ID NO: 621 and a VL encoded by the nucleotide sequence of SEQ ID NO: 625.

In one embodiment, the anti-PD-L1 antibody molecule comprises a heavy chain comprising the amino acid sequence of SEQ ID NO: 608, or an amino acid sequence at least 85%, 90%, 95%, or 99% identical or higher to SEQ ID NO: 608. In one embodiment, the anti-PD-L1 antibody molecule comprises a light chain comprising the amino acid sequence of SEQ ID NO: 618, or an amino acid sequence at least 85%, 90%, 95%, or 99% identical or higher to SEQ ID NO: 618. In one embodiment, the anti-PD-L1 antibody molecule comprises a heavy chain comprising the amino acid sequence of SEQ ID NO: 622, or an amino acid sequence at least 85%, 90%, 95%, or 99% identical or higher to SEQ ID NO: 622. In one embodiment, the anti-PD-L1 antibody molecule comprises a light chain comprising the amino acid sequence of SEQ ID NO: 626, or an amino acid sequence at least 85%, 90%, 95%, or 99% identical or higher to SEQ ID NO: 626. In one embodiment, the anti-PD-L1 antibody molecule comprises a heavy chain comprising the amino acid sequence of SEQ ID NO: 608 and a light chain comprising the amino acid sequence of SEQ ID NO: 618. In one embodiment, the anti-PD-L1 antibody molecule comprises a heavy chain comprising the amino acid sequence of SEQ ID NO: 622 and a light chain comprising the amino acid sequence of SEQ ID NO: 626.

In one embodiment, the antibody molecule comprises a heavy chain encoded by the nucleotide sequence of SEQ ID NO: 615, or a nucleotide sequence at least 85%, 90%, 95%, or 99% identical or higher to SEQ ID NO: 615. In one embodiment, the antibody molecule comprises a light chain encoded by the nucleotide sequence of SEQ ID NO: 619, or a nucleotide sequence at least 85%, 90%, 95%, or 99% identical or higher to SEQ ID NO: 619. In one embodiment, the antibody molecule comprises a heavy chain encoded by the nucleotide sequence of SEQ ID NO: 623, or a nucleotide sequence at least 85%, 90%, 95%, or 99% identical or higher to SEQ ID NO: 623. In one embodiment, the antibody molecule comprises a light chain encoded by the nucleotide sequence of SEQ ID NO: 627, or a nucleotide sequence at least 85%, 90%, 95%, or 99% identical or higher to SEQ ID NO: 627. In one embodiment, the antibody molecule comprises a heavy chain encoded by the nucleotide sequence of SEQ ID NO: 615 and a light chain encoded by the nucleotide sequence of SEQ ID NO: 619. In one embodiment, the antibody molecule comprises a heavy chain encoded by the nucleotide sequence of SEQ ID NO: 623 and a light chain encoded by the nucleotide sequence of SEQ ID NO: 627.

The antibody molecules described herein can be made by vectors, host cells, and methods described in US 2016/0108123, incorporated by reference in its entirety.

TABLE 13
Amino acid and nucleotide sequences of exemplary anti-PD-L1 antibody molecules
BAP058-Clone O HC

| | | |
|---|---|---|
| SEQ ID NO: 601 | HCDR1 | SYWMY |
| (Kabat) | | |
| SEQ ID NO: 602 | HCDR2 | RIDPNSGSTKYNEKFKN |
| (Kabat) | | |
| SEQ ID NO: 603 | HCDR3 | DYRKGLYAMDY |
| (Kabat) | | |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 606 | VH | EVQLVQSGAEVKKPGATVKISCKVSGYTFTSYWMYWV |
| | | RQARGQRLEWIGRIDPNSGSTKYNEKFKNRFTISRDNS |
| | | KNTLYLQMNSLRAEDTAVYYCARDYRKGLYAMDYWG |
| | | QGTTVTVSS |
| SEQ ID NO: 607 | DNA VH | GAAGTGCAGCTGGTGCAGTCAGGCGCCGAAGTGAA |
| | | GAAACCCGGCGCTACCGTGAAGATTAGCTGTAAAGT |
| | | CTCAGGCTACACCTTCACTAGCTACTGGATGTACTG |
| | | GGTCCGACAGGCTAGAGGGCAAAGACTGGAGTGGA |
| | | TCGGTAGAATCGACCCTAATAGCGGCTCTACTAAGTA |
| | | TAACGAGAAGTTTAAGAATAGGTTCACTATTAGTAGG |
| | | GATAACTCTAAGAACACCCTGTACCTGCAGATGAATA |
| | | GCCTGAGAGCCGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| SEQ ID NO: 608 | Heavy | EVQLVQSGAEVKKPGATVKISCKVSGYTFTSYWMYWV |
| | chain | RQARGQRLEWIGRIDPNSGSTKYNEKFKNRFTISRDNS |
| | | KNTLYLQMNSLRAEDTAVYYCARDYRKGLYAMDYWG |
| | | QGTTVTVSSASTKGPSVFPLAPCSRSTSESTAALGCLV |
| | | KDYFPEPVTVSWNSGALTSGVHTFPAVLQSSGLYSLSS |
| | | VVTVPSSSLGTKTYTCNVDHKPSNTKVDKRVESKYGPP |
| | | CPPCPAPEFLGGPSVFLFPPKPKDTLMISRTPEVTCVV |
| | | VDVSQEDPEVQFNWYVDGVEVHNAKTKPREEQFNST |
| | | YRVVSVLTVLHQDWLNGKEYKCKVSNKGLPSSIEKTIS |
| | | KAKGQPREPQVYTLPPSQEEMTKNQVSLTCLVKGFYP |
| | | SDIAVEWESNGQPENNYKTTPPVLDSDGSFFLYSRLTV |
| | | DKSRWQEGNVFSCSVMHEALHNHYTQKSLSLSLG |
| SEQ ID NO: 615 | DNA | GAAGTGCAGCTGGTGCAGTCAGGCGCCGAAGTGAA |
| | heavy | GAAACCCGGCGCTACCGTGAAGATTAGCTGTAAAGT |
| | chain | CTCAGGCTACACCTTCACTAGCTACTGGATGTACTG |
| | | GGTCCGACAGGCTAGAGGGCAAAGACTGGAGTGGA |
| | | TCGGTAGAATCGACCCTAATAGCGGCTCTACTAAGTA |
| | | TAACGAGAAGTTTAAGAATAGGTTCACTATTAGTAGG |
| | | GATAACTCTAAGAACACCCTGTACCTGCAGATGAATA |
| | | GCCTGAGAGCCGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| | | GCTAGCACTAAGGGCCCGTCCGTGTTCCCCCTGGCA |
| | | CCTTGTAGCCGGAGCACTAGCGAATCCACCGCTGCC |
| | | CTCGGCTGCCTGGTCAAGGATTACTTCCCGGAGCCC |
| | | GTGACCGTGTCCTGGAACAGCGGAGCCCTGACCTC |
| | | CGGAGTGCACACCTTCCCCGCTGTGCTGCAGAGCTC |
| | | CGGGCTGTACTCGCTGTCGTCGGTGGTCACGGTGC |
| | | CTTCATCTAGCCTGGGTACCAAGACCTACACTTGCAA |
| | | CGTGGACCACAAGCCTTCCAACACTAAGGTGGACAA |
| | | GCGCGTCGAATCGAAGTACGGCCCACCGTGCCCGC |
| | | CTTGTCCCGCGCCGGAGTTCCTCGGCGGTCCCTCG |
| | | GTCTTTCTGTTCCCACCGAAGCCCAAGGACACTTTGA |
| | | TGATTTCCCGCACCCCTGAAGTGACATGCGTGGTCG |
| | | TGGACGTGTCACAGGAAGATCCGGAGGTGCAGTTCA |
| | | ATTGGTACGTGGATGGCGTCGAGGTGCACAACGCCA |
| | | AAACCAAGCCGAGGGAGGAGCAGTTCAACTCCACTT |
| | | ACCGCGTCGTGTCCGTGCTGACGGTGCTGCATCAG |
| | | GACTGGCTGAACGGGAAGGAGTACAAGTGCAAAGTG |
| | | TCCAACAAGGGACTTCCTAGCTCAATCGAAAAGACC |
| | | ATCTCGAAAGCCAAGGGACAGCCCCGGGAACCCCA |
| | | AGTGTATACCCTGCCACCGAGCCAGGAAGAAATGAC |
| | | TAAGAACCAAGTCTCATTGACTTGCCTTGTGAAGGGC |
| | | TTCTACCCATCGGATATCGCCGTGGAATGGGAGTCC |
| | | AACGGCCAGCCGGAAAACAACTACAAGACCACCCCT |
| | | CCGGTGCTGGACTCAGACGGATCCTTCTTCCTCTAC |
| | | TCGCGGCTGACCGTGGATAAGAGCAGATGGCAGGA |
| | | GGGAAATGTGTTCAGCTGTTCTGTGATGCATGAAGC |
| | | CCTGCACAACCACTACACTCAGAAGTCCCTGTCCCT |
| | | CTCCCTGGGA |

BAP058-Clone O LC

| | | |
|---|---|---|
| SEQ ID NO: 609 | LCDR1 | KASQDVGTAVA |
| (Kabat) | | |
| SEQ ID NO: 610 | LCDR2 | WASTRHT |
| (Kabat) | | |
| SEQ ID NO: 611 | LCDR3 | QQYNSYPLT |
| Kabat) | | |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 616 | VL | AIQLTQSPSSLSASVGDRVTITCKASQDVGTAVAWYLQ |
| | | KPGQSPQLLIYWASTRHTGVPSRFSGSGSGTDFTFTIS |
| | | SLEAEDAATYYCQQYNSYPLTFGQGTKVEIK |
| SEQ ID NO: 617 | DNA VL | GCTATTCAGCTGACTCAGTCACCTAGTAGCCTGAGC |
| | | GCTAGTGTGGGCGATAGAGTGACTATCACCTGTAAA |
| | | GCCTCTCAGGACGTGGGCACCGCCGTGGCCTGGTA |
| | | TCTGCAGAAGCCTGGTCAATCACCTCAGCTGCTGAT |
| | | CTACTGGGCCTCTACTAGACACACCGGCGTGCCCTC |
| | | TAGGTTTAGCGGTAGCGGTAGTGGCACCGACTTCAC |
| | | CTTCACTATCTCTTCACTGGAAGCCGAGGACGCCGC |
| | | TACCTACTACTGTCAGCAGTATAATAGCTACCCCCTG |
| | | ACCTTCGGTCAAGGCACTAAGGTCGAGATTAAG |
| SEQ ID NO: 618 | Light | AIQLTQSPSSLSASVGDRVTITCKASQDVGTAVAWYLQ |
| | chain | KPGQSPQLLIYWASTRHTGVPSRFSGSGSGTDFTFTIS |
| | | SLEAEDAATYYCQQYNSYPLTFGQGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID NO: 619 | DNA light | GCTATTCAGCTGACTCAGTCACCTAGTAGCCTGAGC |
| | chain | GCTAGTGTGGGCGATAGAGTGACTATCACCTGTAAA |
| | | GCCTCTCAGGACGTGGGCACCGCCGTGGCCTGGTA |
| | | TCTGCAGAAGCCTGGTCAATCACCTCAGCTGCTGAT |
| | | CTACTGGGCCTCTACTAGACACACCGGCGTGCCCTC |
| | | TAGGTTTAGCGGTAGCGGTAGTGGCACCGACTTCAC |
| | | CTTCACTATCTCTTCACTGGAAGCCGAGGACGCCGC |
| | | TACCTACTACTGTCAGCAGTATAATAGCTACCCCCTG |
| | | ACCTTCGGTCAAGGCACTAAGGTCGAGATTAAGCGT |
| | | ACGGTGGCCGCTCCCAGCGTGTTCATCTTCCCCCCC |
| | | AGCGACGAGCAGCTGAAGAGCGGCACCGCCAGCGT |
| | | GGTGTGCCTGCTGAACAACTTCTACCCCCGGGAGGC |
| | | CAAGGTGCAGTGGAAGGTGGACAACGCCCTGCAGA |
| | | GCGGCAACAGCCAGGAGAGCGTCACCGAGCAGGAC |
| | | AGCAAGGACTCCACCTACAGCCTGAGCAGCACCCTG |
| | | ACCCTGAGCAAGGCCGACTACGAGAAGCATAAGGTG |
| | | TACGCCTGCGAGGTGACCCACCAGGGCCTGTCCAG |
| | | CCCCGTGACCAAGAGCTTCAACAGGGGCGAGTGC |

BAP058-Clone N HC

| | | |
|---|---|---|
| SEQ ID NO: 601 | HCDR1 | SYWMY |
| (Kabat) | | |
| SEQ ID NO: 602 | HCDR2 | RIDPNSGSTKYNEKFKN |
| (Kabat) | | |
| SEQ ID NO: 603 | HCDR3 | DYRKGLYAMDY |
| (Kabat) | | |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 620 | VH | EVQLVQSGAEVKKPGATVKISCKVSGYTFTSYWMYWV |
| | | RQATGQGLEWMGRIDPNSGSTKYNEKFKNRVTITADK |
| | | STSTAYMELSSLRSEDTAVYYCARDYRKGLYAMDYWG |
| | | QGTTVTVSS |
| SEQ ID NO: 621 | DNA VH | GAAGTGCAGCTGGTGCAGTCAGGCGCCGAAGTGAA |
| | | GAAACCCGGCGCTACCGTGAAGATTAGCTGTAAAGT |
| | | CTCAGGCTACACCTTCACTAGCTACTGGATGTACTG |
| | | GGTCCGACAGGCTACCGGTCAAGGCCTGGAGTGGA |
| | | TGGGTAGAATCGACCCTAATAGCGGCTCTACTAAGT |
| | | ATAACGAGAAGTTTAAGAATAGAGTGACTATCACCGC |
| | | CGATAAGTCTACTAGCACCGCCTATATGGAACTGTCT |
| | | AGCCTGAGATCAGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| SEQ ID NO: 622 | Heavy | EVQLVQSGAEVKKPGATVKISCKVSGYTFTSYWMYWV |
| | chain | RQATGQGLEWMGRIDPNSGSTKYNEKFKNRVTITADK |
| | | STSTAYMELSSLRSEDTAVYYCARDYRKGLYAMDYWG |
| | | QGTTVTVSSASTKGPSVFPLAPCSRSTSESTAALGCLV |
| | | KDYFPEPVTVSWNSGALTSGVHTFPAVLQSSGLYSLSS |
| | | VVTVPSSSLGTKTYTCNVDHKPSNTKVDKRVESKYGPP |
| | | CPPCPAPEFLGGPSVFLFPPKPKDTLMISRTPEVTCVV |
| | | VDVSQEDPEVQFNWYVDGVEVHNAKTKPREEQFNST |
| | | YRVVSVLTVLHQDWLNGKEYKCKVSNKGLPSSIEKTIS |
| | | KAKGQPREPQVYTLPPSQEEMTKNQVSLTCLVKGFYP |
| | | SDIAVEWESNGQPENNYKTTPPVLDSDGSFFLYSRLTV |
| | | DKSRWQEGNVFSCSVMHEALHNHYTQKSLSLSLG |
| SEQ ID NO: 623 | DNA | GAAGTGCAGCTGGTGCAGTCAGGCGCCGAAGTGAA |
| | heavy | GAAACCCGGCGCTACCGTGAAGATTAGCTGTAAAGT |
| | chain | CTCAGGCTACACCTTCACTAGCTACTGGATGTACTG |
| | | GGTCCGACAGGCTACCGGTCAAGGCCTGGAGTGGA |
| | | TGGGTAGAATCGACCCTAATAGCGGCTCTACTAAGT |
| | | ATAACGAGAAGTTTAAGAATAGAGTGACTATCACCGC |
| | | CGATAAGTCTACTAGCACCGCCTATATGGAACTGTCT |
| | | AGCCTGAGATCAGAGGACACCGCCGTCTACTACTGC |
| | | GCTAGAGACTATAGAAAGGGCCTGTACGCTATGGAC |
| | | TACTGGGGTCAAGGCACTACCGTGACCGTGTCTTCA |
| | | GCTAGCACTAAGGGCCCGTCCGTGTTCCCCCTGGCA |
| | | CCTTGTAGCCGGAGCACTAGCGAATCCACCGCTGCC |
| | | CTCGGCTGCCTGGTCAAGGATTACTTCCCGGAGCCC |
| | | GTGACCGTGTCCTGGAACAGCGGAGCCCTGACCTC |
| | | CGGAGTGCACACCTTCCCCGCTGTGCTGCAGAGCTC |
| | | CGGGCTGTACTCGCTGTCGTCGGTGGTCACGGTGC |
| | | CTTCATCTAGCCTGGGTACCAAGACCTACACTTGCAA |
| | | CGTGGACCACAAGCCTTCCAACACTAAGGTGGACAA |
| | | GCGCGTCGAATCGAAGTACGGCCCACCGTGCCCGC |
| | | CTTGTCCCGCGCCGGAGTTCCTCGGCGGTCCCTCG |
| | | GTCTTTCTGTTCCCACCGAAGCCCAAGGACACTTTGA |
| | | TGATTTCCCGCACCCCTGAAGTGACATGCGTGGTCG |
| | | TGGACGTGTCACAGGAAGATCCGGAGGTGCAGTTCA |
| | | ATTGGTACGTGGATGGCGTCGAGGTGCACAACGCCA |
| | | AAACCAAGCCGAGGGAGGAGCAGTTCAACTCCACTT |
| | | ACCGCGTCGTGTCCGTGCTGACGGTGCTGCATCAG |
| | | GACTGGCTGAACGGGAAGGAGTACAAGTGCAAAGTG |
| | | TCCAACAAGGGACTTCCTAGCTCAATCGAAAAGACC |
| | | ATCTCGAAAGCCAAGGGACAGCCCCGGGAACCCCA |
| | | AGTGTATACCCTGCCACCGAGCCAGGAAGAAATGAC |
| | | TAAGAACCAAGTCTCATTGACTTGCCTTGTGAAGGGC |
| | | TTCTACCCATCGGATATCGCCGTGGAATGGGAGTCC |
| | | AACGGCCAGCCGGAAAACAACTACAAGACCACCCCT |
| | | CCGGTGCTGGACTCAGACGGATCCTTCTTCCTCTAC |
| | | TCGCGGCTGACCGTGGATAAGAGCAGATGGCAGGA |
| | | GGGAAATGTGTTCAGCTGTTCTGTGATGCATGAAGC |
| | | CCTGCACAACCACTACACTCAGAAGTCCCTGTCCCT |
| | | CTCCCTGGGA |

BAP058-Clone N LC

| | | |
|---|---|---|
| SEQ ID NO: 609 | LCDR1 | KASQDVGTAVA |
| (Kabat) | | |
| SEQ ID NO: 610 | LCDR2 | WASTRHT |
| (Kabat) | | |
| SEQ ID NO: 611 | LCDR3 | QQYNSYPLT |
| (Kabat) | | |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 624 | VL | DVVMTQSPLSLPVTLGQPASISCKASQDVGTAVAWYQ |
| | | QKPGQAPRLLIYWASTRHTGVPSRFSGSGSGTEFTLTI |
| | | SSLQPDDFATYYCQQYNSYPLTFGQGTKVEIK |
| SEQ ID NO: 625 | DNA VL | GACGTCGTGATGACTCAGTCACCCCTGAGCCTGCCC |
| | | GTGACCCTGGGGCAGCCCGCCTCTATTAGCTGTAAA |
| | | GCCTCTCAGGACGTGGGCACCGCCGTGGCCTGGTA |
| | | TCAGCAGAAGCCAGGGCAAGCCCCTAGACTGCTGAT |
| | | CTACTGGGCCTCTACTAGACACACCGGCGTGCCCTC |
| | | TAGGTTTAGCGGTAGCGGTAGTGGCACCGAGTTCAC |
| | | CCTGACTATCTCTTCACTGCAGCCCGACGACTTCGC |
| | | TACCTACTACTGTCAGCAGTATAATAGCTACCCCCTG |
| | | ACCTTCGGTCAAGGCACTAAGGTCGAGATTAAG |
| SEQ ID NO: 626 | Light | DVVMTQSPLSLPVTLGQPASISCKASQDVGTAVAWYQ |
| | chain | QKPGQAPRLLIYWASTRHTGVPSRFSGSGSGTEFTLTI |
| | | SSLQPDDFATYYCQQYNSYPLTFGQGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| SEQ ID NO: 627 | DNA light | GACGTCGTGATGACTCAGTCACCCCTGAGCCTGCCC |
| | chain | GTGACCCTGGGGCAGCCCGCCTCTATTAGCTGTAAA |
| | | GCCTCTCAGGACGTGGGCACCGCCGTGGCCTGGTA |
| | | TCAGCAGAAGCCAGGGCAAGCCCCTAGACTGCTGAT |
| | | CTACTGGGCCTCTACTAGACACACCGGCGTGCCCTC |
| | | TAGGTTTAGCGGTAGCGGTAGTGGCACCGAGTTCAC |
| | | CCTGACTATCTCTTCACTGCAGCCCGACGACTTCGC |
| | | TACCTACTACTGTCAGCAGTATAATAGCTACCCCCTG |
| | | ACCTTCGGTCAAGGCACTAAGGTCGAGATTAAGCGT |
| | | ACGGTGGCCGCTCCCAGCGTGTTCATCTTCCCCCCC |
| | | AGCGACGAGCAGCTGAAGAGCGGCACCGCCAGCGT |
| | | GGTGTGCCTGCTGAACAACTTCTACCCCCGGGAGGC |
| | | CAAGGTGCAGTGGAAGGTGGACAACGCCCTGCAGA |
| | | GCGGCAACAGCCAGGAGAGCGTCACCGAGCAGGAC |
| | | AGCAAGGACTCCACCTACAGCCTGAGCAGCACCCTG |
| | | ACCCTGAGCAAGGCCGACTACGAGAAGCATAAGGTG |
| | | TACGCCTGCGAGGTGACCCACCAGGGCCTGTCCAG |
| | | CCCCGTGACCAAGAGCTTCAACAGGGGCGAGTGC |

BAP058-Clone O HC

| | | |
|---|---|---|
| SEQ ID NO: 628 | HCDR1 | agctactggatgtac |
| (Kabat) | | |
| SEQ ID NO: 629 | HCDR2 | agaatcgaccctaatagcggctctactaagtataacgagaagtttaagaat |
| (Kabat) | | |
| SEQ ID NO: 630 | HCDR3 | gactatagaaagggcctgtacgctatggactac |
| (Kabat) | | |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |

BAP058-Clone O LC

| | | |
|---|---|---|
| SEQ ID NO: 633 | LCDR1 | aaagcctctcaggacgtgggcaccgccgtggcc |
| (Kabat) | | |
| SEQ ID NO: 634 | LCDR2 | tgggcctctactagacacacc |
| (Kabat) | | |
| SEQ ID NO: 635 | LCDR3 | cagcagtataatagctaccccctgacc |
| (Kabat) | | |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |

BAP058-Clone N HC

| | | |
|---|---|---|
| SEQ ID NO: 628 | HCDR1 | agctactggatgtac |
| (Kabat) | | |
| SEQ ID NO: 629 | HCDR2 | agaatcgaccctaatagcggctctactaagtataacgagaagtttaagaat |
| (Kabat) | | |
| SEQ ID NO: 630 | HCDR3 | gactatagaaagggcctgtacgctatggactac |
| (Kabat) | | |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |

BAP058-Clone N LC

| | | |
|---|---|---|
| SEQ ID NO: 633 | LCDR1 | aaagcctctcaggacgtgggcaccgccgtggcc |
| (Kabat) | | |
| SEQ ID NO: 634 | LCDR2 | tgggcctctactagacacacc |
| (Kabat) | | |
| SEQ ID NO: 635 | LCDR3 | cagcagtataatagctaccccctgacc |
| (Kabat) | | |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |

### Other Exemplary PD-L1 Inhibitors

In some embodiments, the PD-L1 inhibitor is anti-PD-L1 antibody. In some embodiments, the anti-PD-L1 inhibitor is selected from YW243.55.S70, MPDL3280A, MEDI-4736, or MDX-1105MSB-0010718C (also referred to as A09-246-2) disclosed in, e.g., WO 2013/0179174, and having a sequence disclosed herein (or a sequence substantially identical or similar thereto, e.g., a sequence at least 85%, 90%, 95% identical or higher to the sequence specified).

In one embodiment, the PD-L1 inhibitor is MDX-1105. MDX-1105, also known as BMS-936559, is an anti-PD-L1 antibody described in PCT Publication No. WO 2007/005874.

In one embodiment, the PD-L1 inhibitor is YW243.55.S70. The YW243.55.S70 antibody is an anti-PD-L1 described in PCT Publication No. WO 2010/077634.

In one embodiment, the PD-L1 inhibitor is MDPL3280A (Genentech/Roche) also known as Atezolizumabm, RG7446, RO5541267, YW243.55.S70, or TECENTRIQ™. MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906 incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab, e.g., as disclosed in Table 14.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in PCT Publication Nos. WO2010/027827 and WO2011/066342).

In one embodiment the PD-L1 inhibitor is an anti-PD-L1 antibody molecule. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 14.

In one embodiment, the anti-PD-L1 antibody molecule is Durvalumab (Medlmmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 14.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 14.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 14
Amino acid sequences of other exemplary anti-PD-L1 antibody molecules

Atezolizumab

| | | |
|---|---|---|
| SEQ ID NO: | Heavy | EVQLVESGGGLVQPGGSLRLSCAASGFTFSDSWIHWVRQAPGKG |
| 639 | chain | LEWVAWISPYGGSTYYADSVKGRFTISADTSKNTAYLQMNSLRAED |
| | | TAVYYCARRHWPGGFDYWGQGTLVTVSSASTKGPSVFPLAPSSK |
| | | STSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHED |
| | | PEVKFNWYVDGVEVHNAKTKPREEQYASTYRVVSVLTVLHQDWLN |
| | | GKEYKCKVSNKALPAPIEKTISKAKGQPREPQVYTLPPSREEMTKN |
| | | QVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| SEQ ID NO: | Light | DIQMTQSPSSLSASVGDRVTITCRASQDVSTAVAWYQQKPGKAPK |
| 640 | chain | LLIYSASFLYSGVPSRFSGSGSGTDFTLTISSLQPEDFATYYCQQYL |
| | | YHPATFGQGTKVEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |

Avelumab

| | | |
|---|---|---|
| SEQ ID NO: | Heavy | EVQLLESGGGLVQPGGSLRLSCAASGFTFSSYIMMWVRQAPGKGL |
| 641 | chain | EWVSSIYPSGGITFYADTVKGRFTISRDNSKNTLYLQMNSLRAEDTA |
| | | VYYCARIKLGTVTTVDYWGQGTLVTVSSASTKGPSVFPLAPSSKST |
| | | SGGTAALGCLVKDYFPEPVTVSWNSGALTSGVHTFPAVLQSSGLY |
| | | SLSSWTVPSSSLGTQTYICNVNHKPSNTKVDKKVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALPAPIEKTISKAKGQPREPQVYTLPPSRDELTKNQV |
| | | SLTCLVKGFYPSDIAVEWESNGQPENNYKTTPPVLDSDGSFFLYSK |
| | | LTVDKSRWQQGNVFSCSVMHEALHNHYTQKSLSLSPGK |
| SEQ ID NO: | Light | QSALTQPASVSGSPGQSITISCTGTSSDVGGYNYVSWYQQHPGKA |
| 642 | chain | PKLMIYDVSNRPSGVSNRFSGSKSGNTASLTISGLQAEDEADYYCS |
| | | SYTSSSTRVFGTGTKVTVLGQPKANPTVTLFPPSSEELQANKATLV |
| | | CLISDFYPGAVTVAWKADGSPVKAGVETTKPSKQSNNKYAASSYLS |
| | | LTPEQWKSHRSYSCQVTHEGSTVEKTVAPTECS |

Durvalumab

| | | |
|---|---|---|
| SEQ ID NO: | Heavy | EVQLVESGGGLVQPGGSLRLSCAASGFTFSRYWMSWVRQAPGKG |
| 643 | chain | LEWVANIKQDGSEKYYVDSVKGRFTISRDNAKNSLYLQMNSLRAED |
| | | TAVYYCAREGGWFGELAFDYWGQGTLVTVSSASTKGPSVFPLAPS |
| | | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCDK |
| | | THTCPPCPAPEFEGGPSVFLFPPKPKDTLMISRTPEVTCVVVDVSH |
| | | EDPEVKFNWYVDGVEVHNAKTKPREEQYNSTYRVVSVLTVLHQD |
| | | WLNGKEYKCKVSNKALPASIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| SEQ ID NO: | Light | EIVLTQSPGTLSLSPGERATLSCRASQRVSSSYLAWYQQKPGQAP |
| 644 | chain | RLLIYDASSRATGIPDRFSGSGSGTDFTLTISRLEPEDFAVYYCQQY |
| | | GSLPVVTFGQGTKVEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |

BMS-936559

| | | |
|---|---|---|
| SEQ ID NO: | VH | QVQLVQSGAEVKKPGSSVKVSCKTSGDTFSTYAISWVRQAPGQGL |
| 645 | | EWMGGIIPIFGKAHYAQKFQGRVTITADESTSTAYMELSSLRSEDTA |
| | | VYFCARKFHFVSGSPFGMDVWGQGTTVTVSS |
| SEQ ID NO: | VL | EIVLTQSPATLSLSPGERATLSCRASQSVSSYLAWYQQKPGQAPRL |
| 646 | | LIYDASNRATGIPARFSGSGSGTDFTLTISSLEPEDFAVYYCQQRSN |
| | | WPTFGQGTKVEIK |

### LAG-3 Inhibitors

In certain embodiments, the inhibitor of an immune checkpoint molecule is an inhibitor of LAG-3. In some embodiments, the antibody conjugate of the present invention is administered in combination with a LAG-3 inhibitor. In some embodiments, the LAG-3 inhibitor is selected from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 15 (e.g., from the heavy and light chain variable region sequences of BAP050-Clone I or BAP050-Clone J disclosed in Table 15), or encoded by a nucleotide sequence shown in Table 15. In some embodiments, the CDRs are according to the Kabat definition (e.g., as set out in Table 15). In some embodiments, the CDRs are according to the Chothia definition (e.g., as set out in Table 15). In some embodiments, the CDRs are according to the combined CDR definitions of both Kabat and Chothia (e.g., as set out in Table 15). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 15, or encoded by a nucleotide sequence shown in Table 15.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 15.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 15. In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 15.

In one embodiment, the anti-LAG-3 antibody molecule comprises a VH comprising the amino acid sequence of SEQ ID NO: 706, or an amino acid sequence at least 85%, 90%, 95%, or 99% identical or higher to SEQ ID NO: 706. In one embodiment, the anti-LAG-3 antibody molecule comprises a VL comprising the amino acid sequence of SEQ ID NO: 718, or an amino acid sequence at least 85%, 90%, 95%, or 99% identical or higher to SEQ ID NO: 718. In one embodiment, the anti-LAG-3 antibody molecule comprises a VH comprising the amino acid sequence of SEQ ID NO: 724, or an amino acid sequence at least 85%, 90%, 95%, or 99% identical or higher to SEQ ID NO: 724. In one embodiment, the anti-LAG-3 antibody molecule comprises a VL comprising the amino acid sequence of SEQ ID NO: 730, or an amino acid sequence at least 85%, 90%, 95%, or 99% identical or higher to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence at least 85%, 90%, 95%, or 99% identical or higher to SEQ ID NO: 707 or 708. In one embodiment, the antibody molecule comprises a VL encoded by the nucleotide sequence of SEQ ID NO: 719 or 720, or a nucleotide sequence at least 85%, 90%, 95%, or 99% identical or higher to SEQ ID NO: 719 or 720. In one embodiment, the antibody molecule comprises a VH encoded by the nucleotide sequence of SEQ ID NO: 725 or 726, or a nucleotide sequence at least 85%, 90%, 95%, or 99% identical or higher to SEQ ID NO: 725 or 726. In one embodiment, the antibody molecule comprises a VL encoded by the nucleotide sequence of SEQ ID NO: 731 or 732, or a nucleotide sequence at least 85%, 90%, 95%, or 99% identical or higher to SEQ ID NO: 731 or 732. In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732.

In one embodiment, the anti-LAG-3 antibody molecule comprises a heavy chain comprising the amino acid sequence of SEQ ID NO: 709, or an amino acid sequence at least 85%, 90%, 95%, or 99% identical or higher to SEQ ID NO: 709. In one embodiment, the anti-LAG-3 antibody molecule comprises a light chain comprising the amino acid sequence of SEQ ID NO: 721, or an amino acid sequence at least 85%, 90%, 95%, or 99% identical or higher to SEQ ID NO: 721. In one embodiment, the anti-LAG-3 antibody molecule comprises a heavy chain comprising the amino acid sequence of SEQ ID NO: 727, or an amino acid sequence at least 85%, 90%, 95%, or 99% identical or higher to SEQ ID NO: 727. In one embodiment, the anti-LAG-3 antibody molecule comprises a light chain comprising the amino acid sequence of SEQ ID NO: 733, or an amino acid sequence at least 85%, 90%, 95%, or 99% identical or higher to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence at least 85%, 90%, 95%, or 99% identical or higher to SEQ ID NO: 716 or 717. In one embodiment, the antibody molecule comprises a light chain encoded by the nucleotide sequence of SEQ ID NO: 722 or 723, or a nucleotide sequence at least 85%, 90%, 95%, or 99% identical or higher to SEQ ID NO: 722 or 723. In one embodiment, the antibody molecule comprises a heavy chain encoded by the nucleotide sequence of SEQ ID NO: 728 or 729, or a nucleotide sequence at least 85%, 90%, 95%, or 99% identical or higher to SEQ ID NO: 728 or 729. In one embodiment, the antibody molecule comprises a light chain encoded by the nucleotide sequence of SEQ ID NO: 734 or 735, or a nucleotide sequence at least 85%, 90%, 95%, or 99% identical or higher to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 15
Amino acid and nucleotide sequences of exemplary anti-LAG-3 antibody molecules
BAP050-Clone I HC

| | | |
|---|---|---|
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO:706 | VH | QVQLVQSGAEVKKPGASVKVSCKASGFTLTNYGMNWVRQ |
| | | ARGQRLEWIGWINTDTGEPTYADDFKGRFVFSLDTSVSTA |
| | | YLQISSLKAEDTAVYYCARNPPYYYGTNNAEAMDYWGQG |
| | | TTVTVSS |
| SEQ ID NO: 707 | DNA VH | CAAGTGCAGCTGGTGCAGTCGGGAGCCGAAGTGAAGAA |
| | | GCCTGGAGCCTCGGTGAAGGTGTCGTGCAAGGCATCCG |
| | | GATTCACCCTCACCAATTACGGGATGAACTGGGTCAGAC |
| | | AGGCCCGGGGTCAACGGCTGGAGTGGATCGGATGGATT |
| | | AACACCGACACCGGGGAGCCTACCTACGCGGACGATTT |
| | | CAAGGGACGGTTCGTGTTCTCCCTCGACACCTCCGTGT |
| | | CCACCGCCTACCTCCAAATCTCCTCACTGAAAGCGGAG |
| | | GACACCGCCGTGTACTATTGCGCGAGGAACCCGCCCTA |
| | | CTACTACGGAACCAACAACGCCGAAGCCATGGACTACT |
| | | GGGGCCAGGGCACCACTGTGACTGTGTCCAGC |
| SEQ ID NO: 708 | DNA VH | CAGGTGCAGCTGGTGCAGTCTGGCGCCGAAGTGAAGAA |
| | | ACCTGGCGCCTCCGTGAAGGTGTCCTGCAAGGCCTCTG |
| | | GCTTCACCCTGACCAACTACGGCATGAACTGGGTGCGA |
| | | CAGGCCAGGGGCCAGCGGCTGGAATGGATCGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCT |
| SEQ ID NO: 709 | Heavy | QVQLVQSGAEVKKPGASVKVSCKASGFTLTNYGMNWVRQ |
| | chain | ARGQRLEWIGWINTDTGEPTYADDFKGRFVFSLDTSVSTA |
| | | YLQISSLKAEDTAVYYCARNPPYYYGTNNAEAMDYWGQG |
| | | TTVTVSSASTKGPSVFPLAPCSRSTSESTAALGCLVKDYFP |
| | | EPVTVSWNSGALTSGVHTFPAVLQSSGLYSLSSVVTVPSS |
| | | SLGTKTYTCNVDHKPSNTKVDKRVESKYGPPCPPCPAPEF |
| | | LGGPSVFLFPPKPKDTLMISRTPEVTCVVVDVSQEDPEVQF |
| | | NWYVDGVEVHNAKTKPREEQFNSTYRVVSVLTVLHQDWL |
| | | NGKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHN |
| | | HYTQKSLSLSLG |
| SEQ ID NO: 716 | DNA | CAAGTGCAGCTGGTGCAGTCGGGAGCCGAAGTGAAGAA |
| | heavy | GCCTGGAGCCTCGGTGAAGGTGTCGTGCAAGGCATCCG |
| | chain | GATTCACCCTCACCAATTACGGGATGAACTGGGTCAGAC |
| | | AGGCCCGGGGTCAACGGCTGGAGTGGATCGGATGGATT |
| | | AACACCGACACCGGGGAGCCTACCTACGCGGACGATTT |
| | | CAAGGGACGGTTCGTGTTCTCCCTCGACACCTCCGTGT |
| | | CCACCGCCTACCTCCAAATCTCCTCACTGAAAGCGGAG |
| | | GACACCGCCGTGTACTATTGCGCGAGGAACCCGCCCTA |
| | | CTACTACGGAACCAACAACGCCGAAGCCATGGACTACT |
| | | GGGGCCAGGGCACCACTGTGACTGTGTCCAGCGCGTC |
| | | CACTAAGGGCCCGTCCGTGTTCCCCCTGGCACCTTGTA |
| | | GCCGGAGCACTAGCGAATCCACCGCTGCCCTCGGCTGC |
| | | CTGGTCAAGGATTACTTCCCGGAGCCCGTGACCGTGTC |
| | | CTGGAACAGCGGAGCCCTGACCTCCGGAGTGCACACCT |
| | | TCCCCGCTGTGCTGCAGAGCTCCGGGCTGTACTCGCTG |
| | | TCGTCGGTGGTCACGGTGCCTTCATCTAGCCTGGGTAC |
| | | CAAGACCTACACTTGCAACGTGGACCACAAGCCTTCCAA |
| | | CACTAAGGTGGACAAGCGCGTCGAATCGAAGTACGGCC |
| | | CACCGTGCCCGCCTTGTCCCGCGCCGGAGTTCCTCGGC |
| | | GGTCCCTCGGTCTTTCTGTTCCCACCGAAGCCCAAGGA |
| | | CACTTTGATGATTTCCCGCACCCCTGAAGTGACATGCGT |
| | | GGTCGTGGACGTGTCACAGGAAGATCCGGAGGTGCAGT |
| | | TCAATTGGTACGTGGATGGCGTCGAGGTGCACAACGCC |
| | | AAAACCAAGCCGAGGGAGGAGCAGTTCAACTCCACTTA |
| | | CCGCGTCGTGTCCGTGCTGACGGTGCTGCATCAGGACT |
| | | GGCTGAACGGGAAGGAGTACAAGTGCAAAGTGTCCAAC |
| | | AAGGGACTTCCTAGCTCAATCGAAAAGACCATCTCGAAA |
| | | GCCAAGGGACAGCCCCGGGAACCCCAAGTGTATACCCT |
| | | GCCACCGAGCCAGGAAGAAATGACTAAGAACCAAGTCT |
| | | CATTGACTTGCCTTGTGAAGGGCTTCTACCCATCGGATA |
| | | TCGCCGTGGAATGGGAGTCCAACGGCCAGCCGGAAAAC |
| | | AACTACAAGACCACCCCTCCGGTGCTGGACTCAGACGG |
| | | ATCCTTCTTCCTCTACTCGCGGCTGACCGTGGATAAGAG |
| | | CAGATGGCAGGAGGGAAATGTGTTCAGCTGTTCTGTGAT |
| | | GCATGAAGCCCTGCACAACCACTACACTCAGAAGTCCCT |
| | | GTCCCTCTCCCTGGGA |
| SEQ ID NO: 717 | DNA | CAGGTGCAGCTGGTGCAGTCTGGCGCCGAAGTGAAGAA |
| | heavy | ACCTGGCGCCTCCGTGAAGGTGTCCTGCAAGGCCTCTG |
| | chain | GCTTCACCCTGACCAACTACGGCATGAACTGGGTGCGA |
| | | CAGGCCAGGGGCCAGCGGCTGGAATGGATCGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCTGCTTC |
| | | TACCAAGGGGCCCAGCGTGTTCCCCCTGGCCCCCTGCT |
| | | CCAGAAGCACCAGCGAGAGCACAGCCGCCCTGGGCTG |
| | | CCTGGTGAAGGACTACTTCCCCGAGCCCGTGACCGTGT |
| | | CCTGGAACAGCGGAGCCCTGACCAGCGGCGTGCACAC |
| | | CTTCCCCGCCGTGCTGCAGAGCAGCGGCCTGTACAGCC |
| | | TGAGCAGCGTGGTGACCGTGCCCAGCAGCAGCCTGGG |
| | | CACCAAGACCTACACCTGTAACGTGGACCACAAGCCCA |
| | | GCAACACCAAGGTGGACAAGAGGGTGGAGAGCAAGTAC |
| | | GGCCCACCCTGCCCCCCCTGCCCAGCCCCCGAGTTCCT |
| | | GGGCGGACCCAGCGTGTTCCTGTTCCCCCCCAAGCCCA |
| | | AGGACACCCTGATGATCAGCAGAACCCCCGAGGTGACC |
| | | TGTGTGGTGGTGGACGTGTCCCAGGAGGACCCCGAGGT |
| | | CCAGTTCAACTGGTACGTGGACGGCGTGGAGGTGCACA |
| | | ACGCCAAGACCAAGCCCAGAGAGGAGCAGTTTAACAGC |
| | | ACCTACCGGGTGGTGTCCGTGCTGACCGTGCTGCACCA |
| | | GGACTGGCTGAACGGCAAAGAGTACAAGTGTAAGGTCT |
| | | CCAACAAGGGCCTGCCAAGCAGCATCGAAAAGACCATC |
| | | AGCAAGGCCAAGGGCCAGCCTAGAGAGCCCCAGGTCTA |
| | | CACCCTGCCACCCAGCCAAGAGGAGATGACCAAGAACC |
| | | AGGTGTCCCTGACCTGTCTGGTGAAGGGCTTCTACCCA |
| | | AGCGACATCGCCGTGGAGTGGGAGAGCAACGGCCAGC |
| | | CCGAGAACAACTACAAGACCACCCCCCCAGTGCTGGAC |
| | | AGCGACGGCAGCTTCTTCCTGTACAGCAGGCTGACCGT |
| | | GGACAAGTCCAGATGGCAGGAGGGCAACGTCTTTAGCT |
| | | GCTCCGTGATGCACGAGGCCCTGCACAACCACTACACC |
| | | CAGAAGAGCCTGAGCCTGTCCCTGGGC |

BAP050-Clone I LC

| | | |
|---|---|---|
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 718 | VL | DIQMTQSPSSLSASVGDRVTITCSSSQDISNYLNWYLQKPG |
| | | QSPQLLIYYTSTLHLGVPSRFSGSGSGTEFTLTISSLQPDDF |
| | | ATYYCQQYYNLPWTFGQGTKVEIK |
| SEQ ID NO: 719 | DNA VL | GATATTCAGATGACTCAGTCACCTAGTAGCCTGAGCGCT |
| | | AGTGTGGGCGATAGAGTGACTATCACCTGTAGCTCTAGT |
| | | CAGGATATCTCTAACTACCTGAACTGGTATCTGCAGAAG |
| | | CCCGGTCAATCACCTCAGCTGCTGATCTACTACACTAGC |
| | | ACCCTGCACCTGGGCGTGCCCTCTAGGTTTAGCGGTAG |
| | | CGGTAGTGGCACCGAGTTCACCCTGACTATCTCTAGCCT |
| | | GCAGCCCGACGACTTCGCTACCTACTACTGTCAGCAGTA |
| | | CTATAACCTGCCCTGGACCTTCGGTCAAGGCACTAAGGT |
| | | CGAGATTAAG |
| SEQ ID NO: 720 | DNA VL | GACATCCAGATGACCCAGTCCCCCTCCAGCCTGTCTGC |
| | | TTCCGTGGGCGACAGAGTGACCATCACCTGTTCCTCCA |
| | | GCCAGGACATCTCCAACTACCTGAACTGGTATCTGCAGA |
| | | AGCCCGGCCAGTCCCCTCAGCTGCTGATCTACTACACC |
| | | TCCACCCTGCACCTGGGCGTGCCCTCCAGATTTTCCGG |
| | | CTCTGGCTCTGGCACCGAGTTTACCCTGACCATCAGCTC |
| | | CCTGCAGCCCGACGACTTCGCCACCTACTACTGCCAGC |
| | | AGTACTACAACCTGCCCTGGACCTTCGGCCAGGGCACC |
| | | AAGGTGGAAATCAAG |
| SEQ ID NO: 721 | Light | DIQMTQSPSSLSASVGDRVTITCSSSQDISNYLNWYLQKPG |
| | chain | QSPQLLIYYTSTLHLGVPSRFSGSGSGTEFTLTISSLQPDDF |
| | | ATYYCQQYYNLPWTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| SEQ ID NO: 722 | DNA | GATATTCAGATGACTCAGTCACCTAGTAGCCTGAGCGCT |
| | light | AGTGTGGGCGATAGAGTGACTATCACCTGTAGCTCTAGT |
| | chain | CAGGATATCTCTAACTACCTGAACTGGTATCTGCAGAAG |
| | | CCCGGTCAATCACCTCAGCTGCTGATCTACTACACTAGC |
| | | ACCCTGCACCTGGGCGTGCCCTCTAGGTTTAGCGGTAG |
| | | CGGTAGTGGCACCGAGTTCACCCTGACTATCTCTAGCCT |
| | | GCAGCCCGACGACTTCGCTACCTACTACTGTCAGCAGTA |
| | | CTATAACCTGCCCTGGACCTTCGGTCAAGGCACTAAGGT |
| | | CGAGATTAAGCGTACGGTGGCCGCTCCCAGCGTGTTCA |
| | | TCTTCCCCCCCAGCGACGAGCAGCTGAAGAGCGGCACC |
| | | GCCAGCGTGGTGTGCCTGCTGAACAACTTCTACCCCCG |
| | | GGAGGCCAAGGTGCAGTGGAAGGTGGACAACGCCCTG |
| | | CAGAGCGGCAACAGCCAGGAGAGCGTCACCGAGCAGG |
| | | ACAGCAAGGACTCCACCTACAGCCTGAGCAGCACCCTG |
| | | ACCCTGAGCAAGGCCGACTACGAGAAGCATAAGGTGTA |
| | | CGCCTGCGAGGTGACCCACCAGGGCCTGTCCAGCCCC |
| | | GTGACCAAGAGCTTCAACAGGGGCGAGTGC |
| SEQ ID NO: 723 | DNA | GACATCCAGATGACCCAGTCCCCCTCCAGCCTGTCTGC |
| | light | TTCCGTGGGCGACAGAGTGACCATCACCTGTTCCTCCA |
| | chain | GCCAGGACATCTCCAACTACCTGAACTGGTATCTGCAGA |
| | | AGCCCGGCCAGTCCCCTCAGCTGCTGATCTACTACACC |
| | | TCCACCCTGCACCTGGGCGTGCCCTCCAGATTTTCCGG |
| | | CTCTGGCTCTGGCACCGAGTTTACCCTGACCATCAGCTC |
| | | CCTGCAGCCCGACGACTTCGCCACCTACTACTGCCAGC |
| | | AGTACTACAACCTGCCCTGGACCTTCGGCCAGGGCACC |
| | | AAGGTGGAAATCAAGCGTACGGTGGCCGCTCCCAGCGT |
| | | GTTCATCTTCCCCCCAAGCGACGAGCAGCTGAAGAGCG |
| | | GCACCGCCAGCGTGGTGTGTCTGCTGAACAACTTCTAC |
| | | CCCAGGGAGGCCAAGGTGCAGTGGAAGGTGGACAACG |
| | | CCCTGCAGAGCGGCAACAGCCAGGAGAGCGTCACCGA |
| | | GCAGGACAGCAAGGACTCCACCTACAGCCTGAGCAGCA |
| | | CCCTGACCCTGAGCAAGGCCGACTACGAGAAGCACAAG |
| | | GTGTACGCCTGTGAGGTGACCCACCAGGGCCTGTCCAG |
| | | CCCCGTGACCAAGAGCTTCAACAGGGGCGAGTGC |

BAP050-Clone J HC

| | | |
|---|---|---|
| SEQ ID NO: 701 | HCDR1 | NYGMN |
| (Kabat) | | |
| SEQ ID NO: 702 | HCDR2 | WINTDTGEPTYADDFKG |
| (Kabat) | | |
| SEQ ID NO: 703 | HCDR3 | NPPYYYGTNNAEAMDY |
| (Kabat) | | |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 724 | VH | QVQLVQSGAEVKKPGASVKVSCKASGFTLTNYGMNWVRQ |
| | | APGQGLEWMGWINTDTGEPTYADDFKGRFVFSLDTSVST |
| | | AYLQISSLKAEDTAVYYCARNPPYYYGTNNAEAMDYWGQ |
| | | GTTVTVSS |
| SEQ ID NO: 725 | DNA VH | CAGGTGCAGCTGGTGCAGTCAGGCGCCGAAGTGAAGAA |
| | | ACCCGGCGCTAGTGTGAAAGTCAGCTGTAAAGCTAGTG |
| | | GCTTCACCCTGACTAACTACGGGATGAACTGGGTCCGC |
| | | CAGGCCCCAGGTCAAGGCCTCGAGTGGATGGGCTGGAT |
| | | TAACACCGACACCGGCGAGCCTACCTACGCCGACGACT |
| | | TTAAGGGCAGATTCGTGTTTAGCCTGGACACTAGTGTGT |
| | | CTACCGCCTACCTGCAGATCTCTAGCCTGAAGGCCGAG |
| | | GACACCGCCGTCTACTACTGCGCTAGAAACCCCCCCTA |
| | | CTACTACGGCACTAACAACGCCGAGGCTATGGACTACT |
| | | GGGGTCAAGGCACTACCGTGACCGTGTCTAGC |
| SEQ ID NO: 726 | DNA VH | CAGGTGCAGCTGGTGCAGTCTGGCGCCGAAGTGAAGAA |
| | | ACCTGGCGCCTCCGTGAAGGTGTCCTGCAAGGCCTCTG |
| | | GCTTCACCCTGACCAACTACGGCATGAACTGGGTGCGA |
| | | CAGGCCCCTGGACAGGGCCTGGAATGGATGGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCT |
| SEQ ID NO: 727 | Heavy | QVQLVQSGAEVKKPGASVKVSCKASGFTLTNYGMNWVRQ |
| | chain | APGQGLEWMGWINTDTGEPTYADDFKGRFVFSLDTSVST |
| | | AYLQISSLKAEDTAVYYCARNPPYYYGTNNAEAMDYWGQ |
| | | GTTVTVSSASTKGPSVFPLAPCSRSTSESTAALGCLVKDYF |
| | | PEPVTVSWNSGALTSGVHTFPAVLQSSGLYSLSSVVTVPS |
| | | SSLGTKTYTCNVDHKPSNTKVDKRVESKYGPPCPPCPAPE |
| | | FLGGPSVFLFPPKPKDTLMSRTPEVTCVVVDVSQEDPEVQ |
| | | FNWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKTT |
| | | PPVLDSDGSFFLYSRLTVDKSRWQEGNVFSCSVMHEALH |
| | | NHYTQKSLSLSLG |
| SEQ ID NO: 728 | DNA | CAGGTGCAGCTGGTGCAGTCAGGCGCCGAAGTGAAGAA |
| | heavy | ACCCGGCGCTAGTGTGAAAGTCAGCTGTAAAGCTAGTG |
| | chain | GCTTCACCCTGACTAACTACGGGATGAACTGGGTCCGC |
| | | CAGGCCCCAGGTCAAGGCCTCGAGTGGATGGGCTGGAT |
| | | TAACACCGACACCGGCGAGCCTACCTACGCCGACGACT |
| | | TTAAGGGCAGATTCGTGTTTAGCCTGGACACTAGTGTGT |
| | | CTACCGCCTACCTGCAGATCTCTAGCCTGAAGGCCGAG |
| | | GACACCGCCGTCTACTACTGCGCTAGAAACCCCCCCTA |
| | | CTACTACGGCACTAACAACGCCGAGGCTATGGACTACT |
| | | GGGGTCAAGGCACTACCGTGACCGTGTCTAGCGCTAGC |
| | | ACTAAGGGCCCGTCCGTGTTCCCCCTGGCACCTTGTAG |
| | | CCGGAGCACTAGCGAATCCACCGCTGCCCTCGGCTGCC |
| | | TGGTCAAGGATTACTTCCCGGAGCCCGTGACCGTGTCC |
| | | TGGAACAGCGGAGCCCTGACCTCCGGAGTGCACACCTT |
| | | CCCCGCTGTGCTGCAGAGCTCCGGGCTGTACTCGCTGT |
| | | CGTCGGTGGTCACGGTGCCTTCATCTAGCCTGGGTACC |
| | | AAGACCTACACTTGCAACGTGGACCACAAGCCTTCCAAC |
| | | ACTAAGGTGGACAAGCGCGTCGAATCGAAGTACGGCCC |
| | | ACCGTGCCCGCCTTGTCCCGCGCCGGAGTTCCTCGGCG |
| | | GTCCCTCGGTCTTTCTGTTCCCACCGAAGCCCAAGGACA |
| | | CTTTGATGATTTCCCGCACCCCTGAAGTGACATGCGTGG |
| | | TCGTGGACGTGTCACAGGAAGATCCGGAGGTGCAGTTC |
| | | AATTGGTACGTGGATGGCGTCGAGGTGCACAACGCCAA |
| | | AACCAAGCCGAGGGAGGAGCAGTTCAACTCCACTTACC |
| | | GCGTCGTGTCCGTGCTGACGGTGCTGCATCAGGACTGG |
| | | CTGAACGGGAAGGAGTACAAGTGCAAAGTGTCCAACAA |
| | | GGGACTTCCTAGCTCAATCGAAAAGACCATCTCGAAAGC |
| | | CAAGGGACAGCCCCGGGAACCCCAAGTGTATACCCTGC |
| | | CACCGAGCCAGGAAGAAATGACTAAGAACCAAGTCTCAT |
| | | TGACTTGCCTTGTGAAGGGCTTCTACCCATCGGATATCG |
| | | CCGTGGAATGGGAGTCCAACGGCCAGCCGGAAAACAAC |
| | | TACAAGACCACCCCTCCGGTGCTGGACTCAGACGGATC |
| | | CTTCTTCCTCTACTCGCGGCTGACCGTGGATAAGAGCAG |
| | | ATGGCAGGAGGGAAATGTGTTCAGCTGTTCTGTGATGCA |
| | | TGAAGCCCTGCACAACCACTACACTCAGAAGTCCCTGTC |
| | | CCTCTCCCTGGGA |
| SEQ ID NO: 729 | DNA | CAGGTGCAGCTGGTGCAGTCTGGCGCCGAAGTGAAGAA |
| | heavy | ACCTGGCGCCTCCGTGAAGGTGTCCTGCAAGGCCTCTG |
| | chain | GCTTCACCCTGACCAACTACGGCATGAACTGGGTGCGA |
| | | CAGGCCCCTGGACAGGGCCTGGAATGGATGGGCTGGA |
| | | TCAACACCGACACCGGCGAGCCTACCTACGCCGACGAC |
| | | TTCAAGGGCAGATTCGTGTTCTCCCTGGACACCTCCGTG |
| | | TCCACCGCCTACCTGCAGATCTCCAGCCTGAAGGCCGA |
| | | GGATACCGCCGTGTACTACTGCGCCCGGAACCCCCCTT |
| | | ACTACTACGGCACCAACAACGCCGAGGCCATGGACTAT |
| | | TGGGGCCAGGGCACCACCGTGACCGTGTCCTCTGCTTC |
| | | TACCAAGGGGCCCAGCGTGTTCCCCCTGGCCCCCTGCT |
| | | CCAGAAGCACCAGCGAGAGCACAGCCGCCCTGGGCTG |
| | | CCTGGTGAAGGACTACTTCCCCGAGCCCGTGACCGTGT |
| | | CCTGGAACAGCGGAGCCCTGACCAGCGGCGTGCACAC |
| | | CTTCCCCGCCGTGCTGCAGAGCAGCGGCCTGTACAGCC |
| | | TGAGCAGCGTGGTGACCGTGCCCAGCAGCAGCCTGGG |
| | | CACCAAGACCTACACCTGTAACGTGGACCACAAGCCCA |
| | | GCAACACCAAGGTGGACAAGAGGGTGGAGAGCAAGTAC |
| | | GGCCCACCCTGCCCCCCCTGCCCAGCCCCCGAGTTCCT |
| | | GGGCGGACCCAGCGTGTTCCTGTTCCCCCCCAAGCCCA |
| | | AGGACACCCTGATGATCAGCAGAACCCCCGAGGTGACC |
| | | TGTGTGGTGGTGGACGTGTCCCAGGAGGACCCCGAGGT |
| | | CCAGTTCAACTGGTACGTGGACGGCGTGGAGGTGCACA |
| | | ACGCCAAGACCAAGCCCAGAGAGGAGCAGTTTAACAGC |
| | | ACCTACCGGGTGGTGTCCGTGCTGACCGTGCTGCACCA |
| | | GGACTGGCTGAACGGCAAAGAGTACAAGTGTAAGGTCT |
| | | CCAACAAGGGCCTGCCAAGCAGCATCGAAAAGACCATC |
| | | AGCAAGGCCAAGGGCCAGCCTAGAGAGCCCCAGGTCTA |
| | | CACCCTGCCACCCAGCCAAGAGGAGATGACCAAGAACC |
| | | AGGTGTCCCTGACCTGTCTGGTGAAGGGCTTCTACCCA |
| | | AGCGACATCGCCGTGGAGTGGGAGAGCAACGGCCAGC |
| | | CCGAGAACAACTACAAGACCACCCCCCCAGTGCTGGAC |
| | | AGCGACGGCAGCTTCTTCCTGTACAGCAGGCTGACCGT |
| | | GGACAAGTCCAGATGGCAGGAGGGCAACGTCTTTAGCT |
| | | GCTCCGTGATGCACGAGGCCCTGCACAACCACTACACC |
| | | CAGAAGAGCCTGAGCCTGTCCCTGGGC |

BAP050-Clone J LC

| | | |
|---|---|---|
| SEQ ID NO: 710 | LCDR1 | SSSQDISNYLN |
| (Kabat) | | |
| SEQ ID NO: 711 | LCDR2 | YTSTLHL |
| (Kabat) | | |
| SEQ ID NO: 712 | LCDR3 | QQYYNLPWT |
| (Kabat) | | |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 730 | VL | DIQMTQSPSSLSASVGDRVTITCSSSQDISNYLNWYQQKP |
| | | GKAPKLLIYYTSTLHLGIPPRFSGSGYGTDFTLTINNIESEDA |
| | | AYYFCQQYYNLPWTFGQGTKVEIK |
| SEQ ID NO: 731 | DNA VL | GATATTCAGATGACTCAGTCACCTAGTAGCCTGAGCGCT |
| | | AGTGTGGGCGATAGAGTGACTATCACCTGTAGCTCTAGT |
| | | CAGGATATCTCTAACTACCTGAACTGGTATCAGCAGAAG |
| | | CCCGGTAAAGCCCCTAAGCTGCTGATCTACTACACTAGC |
| | | ACCCTGCACCTGGGAATCCCCCCTAGGTTTAGCGGTAG |
| | | CGGCTACGGCACCGACTTCACCCTGACTATTAACAATAT |
| | | CGAGTCAGAGGACGCCGCCTACTACTTCTGTCAGCAGT |
| | | ACTATAACCTGCCCTGGACCTTCGGTCAAGGCACTAAGG |
| | | TCGAGATTAAG |
| SEQ ID NO: 732 | DNA VL | GACATCCAGATGACCCAGTCCCCCTCCAGCCTGTCTGC |
| | | TTCCGTGGGCGACAGAGTGACCATCACCTGTTCCTCCA |
| | | GCCAGGACATCTCCAACTACCTGAACTGGTATCAGCAGA |
| | | AGCCCGGCAAGGCCCCCAAGCTGCTGATCTACTACACC |
| | | TCCACCCTGCACCTGGGCATCCCCCCTAGATTCTCCGG |
| | | CTCTGGCTACGGCACCGACTTCACCCTGACCATCAACAA |
| | | CATCGAGTCCGAGGACGCCGCCTACTACTTCTGCCAGC |
| | | AGTACTACAACCTGCCCTGGACCTTCGGCCAGGGCACC |
| | | AAGGTGGAAATCAAG |
| SEQ ID NO: 733 | Light | DIQMTQSPSSLSASVGDRVTITCSSSQDISNYLNWYQQKP |
| | chain | GKAPKLLIYYTSTLHLGIPPRFSGSGYGTDFTLTINNIESEDA |
| | | AYYFCQQYYNLPWTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |
| SEQ ID NO: 734 | DNA | GATATTCAGATGACTCAGTCACCTAGTAGCCTGAGCGCT |
| | light | AGTGTGGGCGATAGAGTGACTATCACCTGTAGCTCTAGT |
| | chain | CAGGATATCTCTAACTACCTGAACTGGTATCAGCAGAAG |
| | | CCCGGTAAAGCCCCTAAGCTGCTGATCTACTACACTAGC |
| | | ACCCTGCACCTGGGAATCCCCCCTAGGTTTAGCGGTAG |
| | | CGGCTACGGCACCGACTTCACCCTGACTATTAACAATAT |
| | | CGAGTCAGAGGACGCCGCCTACTACTTCTGTCAGCAGT |
| | | ACTATAACCTGCCCTGGACCTTCGGTCAAGGCACTAAGG |
| | | TCGAGATTAAGCGTACGGTGGCCGCTCCCAGCGTGTTC |
| | | ATCTTCCCCCCCAGCGACGAGCAGCTGAAGAGCGGCAC |
| | | CGCCAGCGTGGTGTGCCTGCTGAACAACTTCTACCCCC |
| | | GGGAGGCCAAGGTGCAGTGGAAGGTGGACAACGCCCT |
| | | GCAGAGCGGCAACAGCCAGGAGAGCGTCACCGAGCAG |
| | | GACAGCAAGGACTCCACCTACAGCCTGAGCAGCACCCT |
| | | GACCCTGAGCAAGGCCGACTACGAGAAGCATAAGGTGT |
| | | ACGCCTGCGAGGTGACCCACCAGGGCCTGTCCAGCCC |
| | | CGTGACCAAGAGCTTCAACAGGGGCGAGTGC |
| SEQ ID NO: 735 | DNA | GACATCCAGATGACCCAGTCCCCCTCCAGCCTGTCTGC |
| | light | TTCCGTGGGCGACAGAGTGACCATCACCTGTTCCTCCA |
| | chain | GCCAGGACATCTCCAACTACCTGAACTGGTATCAGCAGA |
| | | AGCCCGGCAAGGCCCCCAAGCTGCTGATCTACTACACC |
| | | TCCACCCTGCACCTGGGCATCCCCCCTAGATTCTCCGG |
| | | CTCTGGCTACGGCACCGACTTCACCCTGACCATCAACAA |
| | | CATCGAGTCCGAGGACGCCGCCTACTACTTCTGCCAGC |
| | | AGTACTACAACCTGCCCTGGACCTTCGGCCAGGGCACC |
| | | AAGGTGGAAATCAAGCGTACGGTGGCCGCTCCCAGCGT |
| | | GTTCATCTTCCCCCCAAGCGACGAGCAGCTGAAGAGCG |
| | | GCACCGCCAGCGTGGTGTGTCTGCTGAACAACTTCTAC |
| | | CCCAGGGAGGCCAAGGTGCAGTGGAAGGTGGACAACG |
| | | CCCTGCAGAGCGGCAACAGCCAGGAGAGCGTCACCGA |
| | | GCAGGACAGCAAGGACTCCACCTACAGCCTGAGCAGCA |
| | | CCCTGACCCTGAGCAAGGCCGACTACGAGAAGCACAAG |
| | | GTGTACGCCTGTGAGGTGACCCACCAGGGCCTGTCCAG |
| | | CCCCGTGACCAAGAGCTTCAACAGGGGCGAGTGC |

BAP050-Clone I HC

| | | |
|---|---|---|
| SEQ ID NO: 736 | HCDR1 | AATTACGGGATGAAC |
| (Kabat) | | |
| SEQ ID NO: 737 | HCDR1 | AACTACGGCATGAAC |
| (Kabat) | | |
| SEQ ID NO: 738 | HCDR2 | TGGATTAACACCGACACCGGGGAGCCTACCTACGCGGA |
| (Kabat) | | CGATTTCAAGGGA |
| SEQ ID NO: 739 | HCDR2 | TGGATCAACACCGACACCGGCGAGCCTACCTACGCCGA |
| (Kabat) | | CGACTTCAAGGGC |
| SEQ ID NO: 740 | HCDR3 | AACCCGCCCTACTACTACGGAACCAACAACGCCGAAGC |
| (Kabat) | | CATGGACTAC |
| SEQ ID NO: 741 | HCDR3 | AACCCCCCTTACTACTACGGCACCAACAACGCCGAGGC |
| (Kabat) | | |
| CATGGACTAT | | |
| SEQ ID NO: 742 | HCDR1 | GGATTCACCCTCACCAATTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 740 | HCDR3 | AACCCGCCCTACTACTACGGAACCAACAACGCCGAAGC |
| (Chothia) | | CATGGACTAC |
| SEQ ID NO: 741 | HCDR3 | AACCCCCCTTACTACTACGGCACCAACAACGCCGAGGC |
| (Chothia) | | CATGGACTAT |

BAP050-Clone I LC

| | | |
|---|---|---|
| SEQ ID NO: 746 | LCDR1 | AGCTCTAGTCAGGATATCTCTAACTACCTGAAC |
| (Kabat) | | |
| SEQ ID NO: 747 | LCDR1 | TCCTCCAGCCAGGACATCTCCAACTACCTGAAC |
| (Kabat) | | |
| SEQ ID NO: 748 | LCDR2 | TACACTAGCACCCTGCACCTG |
| (Kabat) | | |
| SEQ ID NO: 749 | LCDR2 | TACACCTCCACCCTGCACCTG |
| (Kabat) | | |
| SEQ ID NO: 750 | LCDR3 | CAGCAGTACTATAACCTGCCCTGGACC |
| (Kabat) | | |
| SEQ ID NO: 751 | LCDR3 | CAGCAGTACTACAACCTGCCCTGGACC |
| (Kabat) | | |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |
| BAP050-Clone J HC | | |
| SEQ ID NO: 758 | HCDR1 | AACTACGGGATGAAC |
| (Kabat) | | |
| SEQ ID NO: 737 | HCDR1 | AACTACGGCATGAAC |
| (Kabat) | | |
| SEQ ID NO: 759 | HCDR2 | TGGATTAACACCGACACCGGCGAGCCTACCTACGCCGA |
| (Kabat) | | CGACTTTAAGGGC |
| SEQ ID NO: 739 | HCDR2 | TGGATCAACACCGACACCGGCGAGCCTACCTACGCCGA |
| (Kabat) | | CGACTTCAAGGGC |
| SEQ ID NO: 760 | HCDR3 | AACCCCCCCTACTACTACGGCACTAACAACGCCGAGGC |
| (Kabat) | | TATGGACTAC |
| SEQ ID NO: 741 | HCDR3 | AACCCCCCTTACTACTACGGCACCAACAACGCCGAGGC |
| (Kabat) | | CATGGACTAT |
| SEQ ID NO: 761 | HCDR1 | GGCTTCACCCTGACTAACTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 760 | HCDR3 | AACCCCCCCTACTACTACGGCACTAACAACGCCGAGGC |
| (Chothia) | | TATGGACTAC |
| SEQ ID NO: 741 | HCDR3 | AACCCCCCTTACTACTACGGCACCAACAACGCCGAGGC |
| (Chothia) | | CATGGACTAT |

BAP050-Clone J LC

| SEQ ID NO: 746 | LCDR1 | AGCTCTAGTCAGGATATCTCTAACTACCTGAAC | |
|---|---|---|---|
| (Kabat) | | | |
| SEQ | ID NO: 747 | LCDR1 | TCCTCCAGCCAGGACATCTCCAACTACCTGAAC |
| (Kabat) | | | |
| SEQ | ID NO: 748 | LCDR2 | TACACTAGCACCCTGCACCTG |
| (Kabat) | | | |
| SEQ | ID NO: 749 | LCDR2 | TACACCTCCACCCTGCACCTG |
| (Kabat) | | | |
| SEQ | ID NO: 750 | LCDR3 | CAGCAGTACTATAACCTGCCCTGGACC |
| (Kabat) | | | |
| SEQ | ID NO: 751 | LCDR3 | CAGCAGTACTACAACCTGCCCTGGACC |
| (Kabat) | | | |
| SEQ | ID NO: 752 | LCDR1 | AGTCAGGATATCTCTAACTAC |
| (Chothia) | | | |
| SEQ | ID NO: 753 | LCDR1 | AGCCAGGACATCTCCAACTAC |
| (Chothia) | | | |
| SEQ | ID NO: 754 | LCDR2 | TACACTAGC |
| (Chothia) | | | |
| SEQ | ID NO: 755 | LCDR2 | TACACCTCC |
| (Chothia) | | | |
| SEQ | ID NO: 756 | LCDR3 | TACTATAACCTGCCCTGG |
| (Chothia) | | | |
| SEQ | ID NO: 757 | LCDR3 | TACTACAACCTGCCCTGG |
| (Chothia) | | | |

### Other Exemplary LAG-3 Inhibitors

In one embodiment, the LAG-3 inhibitor is an anti-LAG-3 antibody molecule. In one embodiment, the LAG-3 inhibitor is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 16.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 16.

In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 16
Amino acid sequences of other exemplary
anti-LAG-3 antibody molecules

BMS-986016

| | | |
|---|---|---|
| SEQ | Heavy | QVQLQQWGAGLLKPSETLSLTCAVYGGSFSDYYWNW |
| ID | chain | IRQPPGKGLEWIGEINHRGSTNSNPSLKSRVTLSLD |
| NO: | | TSKNQFSLKLRSVTAADTAVYYCAFGYSDYEYNWFD |
| 762 | | PWGQGTLVTVSSASTKGPSVFPLAPCSRSTSESTAA |
| | | LGCLVKDYFPEPVTVSWNSGALTSGVHTFPAVLQSS |
| | | GLYSLSSVVTVPSSSLGTKTYTCNVDHKPSNTKVDK |
| | | RVESKYGPPCPPCPAPEFLGGPSVFLFPPKPKDTLM |
| | | ISRTPEVTCVVVDVSQEDPEVQFNWYVDGVEVHNAK |
| | | TKPREEQFNSTYRVVSVLTVLHQDWLNGKEYKCKVS |
| | | NKGLPSSIEKTISKAKGQPREPQVYTLPPSQEEMTK |
| | | NQVSLTCLVKGFYPSDIAVEWESNGQPENNYKTTPP |
| | | VLDSDGSFFLYSRLTVDKSRWQEGNVFSCSVMHEAL |
| | | HNHYTQKSLSLSLGK |
| SEQ | Light | EIVLTQSPATLSLSPGERATLSCRASQSISSYLAWY |
| ID | chain | QQKPGQAPRLLIYDASNRATGIPARFSGSGSGTDFT |
| NO: | | LTISSLEPEDFAVYYCQQRSNWPLTFGQGTNLEIKR |
| 763 | | TVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSFNRGEC |

IMP731

| | | |
|---|---|---|
| SEQ | Heavy | QVQLKESGPGLVAPSQSLSITCTVSGFSLTAYGVNW |
| ID | chain | VRQPPGKGLEWLGMIWDDGSTDYNSALKSRLSISKD |
| NO: | | NSKSQVFLKMNSLQTDDTARYYCAREGDVAFDYWGQ |
| 764 | | GTTLTVSSASTKGPSVFPLAPSSKSTSGGTAALGCL |
| | | VKDYFPEPVTVSWNSGALTSGVHTFPAVLQSSGLYS |
| | | LSSVVTVPSSSLGTQTYICNVNHKPSNTKVDKKVEP |
| | | KSCDKTHTCPPCPAPELLGGPSVFLFPPKPKDTLMI |
| | | SRTPEVTCVVVDVSHEDPEVKFNWYVDGVEVHNAKT |
| | | KPREEQYNSTYRVVSVLTVLHQDWLNGKEYKCKVSN |
| | | KALPAPIEKTISKAKGQPREPQVYTLPPSRDELTKN |
| | | QVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPGK |
| SEQ | Light | DIVMTQSPSSLAVSVGQKVTMSCKSSQSLLNGSNQK |
| ID | chain | NYLAWYQQKPGQSPKLLVYFASTRDSGVPDRFIGSG |
| NO: | | SGTDFTLTISSVQAEDLADYFCLQHFGTPPTFGGGT |
| 765 | | KLEIKRTVAAPSVFIFPPSDEQLKSGTASVVCLLNN |
| | | FYPREAKVQWKVDNALQSGNSQESVTEQDSKDSTYS |
| | | LSSTLTLSKADYEKHKVYACEVTHQGLSSPVTKSFN |
| | | RGEC |

### TIM-3 Inhibitors

In certain embodiments, the inhibitor of an immune checkpoint molecule is an inhibitor of TIM-3. In some embodiments, the antibody conjugate of the present invention is administered in combination with a TIM-3 inhibitor. In some embodiments, the TIM-3 inhibitor is MGB453 (Novartis) or TSR-022 (Tesaro).

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 17 (e.g., from the heavy and light chain variable region sequences of ABTIM3-hum11 or ABTIM3-hum03 disclosed in Table 17), or encoded by a nucleotide sequence shown in Table 17. In some embodiments, the CDRs are according to the Kabat definition (e.g., as set out in Table 17). In some embodiments, the CDRs are according to the Chothia definition (e.g., as set out in Table 17). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 17, or encoded by a nucleotide sequence shown in Table 17.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 17. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 17.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 17
Amino acid and nucleotide sequences of
exemplary anti-TIM-3 antibody molecules

ABTIM3-hum11

| | | |
|---|---|---|
| SEQ ID | HCDR1 | SYNMH |
| NO: 801 | | |
| (Kabat) | | |
| SEQ ID | HCDR2 | DIYPGNGDTSYNQKFKG |
| NO: 802 | | |
| (Kabat) | | |
| SEQ ID | HCDR3 | VGGAFPMDY |
| NO: 803 | | |
| (Kabat) | | |
| SEQ ID | HCDR1 | GYTFTSY |

NO: 804

| | | |
|---|---|---|
| (Chothia) | | |
| SEQ ID | HCDR2 | YPGNGD |
| NO: 805 | | |
| (Chothia) | | |
| SEQ ID | HCDR3 | VGGAFPMDY |
| NO: 803 | | |
| (Chothia) | | |
| SEQ ID | VH | QVQLVQSGAEVKKPGSSVKVSCKASGYTFTS |
| NO: 806 | | YNMHWVRQAPGQGLEWMGDIYPGNGDTSYNQ |
| | | KFKGRVTITADKSTSTVYMELSSLRSEDTAV |
| | | YYCARVGGAFPMDYWGQGTTVTVSS |
| SEQ ID | DNA VH | CAGGTGCAGCTGGTGCAGTCAGGCGCCGAAG |
| NO: 807 | | TGAAGAAACCCGGCTCTAGCGTGAAAGTTTC |
| | | TTGTAAAGCTAGTGGCTACACCTTCACTAGC |
| | | TATAATATGCACTGGGTTCGCCAGGCCCCAG |
| | | GGCAAGGCCTCGAGTGGATGGGCGATATCTA |
| | | CCCCGGGAACGGCGACACTAGTTATAATCAG |
| | | AAGTTTAAGGGTAGAGTCACTATCACCGCCG |
| | | ATAAGTCTACTAGCACCGTCTATATGGAACT |
| | | GAGTTCCCTGAGGTCTGAGGACACCGCCGTC |
| | | TACTACTGCGCTAGAGTGGGCGGAGCCTTCC |
| | | CTATGGACTACTGGGGTCAAGGCACTACCGT |
| | | GACCGTGTCTAGC |
| SEQ ID | Heavy | QVQLVQSGAEVKKPGSSVKVSCKASGYTFTS |
| NO: 808 | chain | YNMHWVRQAPGQGLEWMGDIYPGNGDTSYNQ |
| | | KFKGRVTITADKSTSTVYMELSSLRSEDTAV |
| | | YYCARVGGAFPMDYWGQGTTVTVSSASTKGP |
| | | SVFPLAPCSRSTSESTAALGCLVKDYFPEPV |
| | | TVSWNSGALTSGVHTFPAVLQSSGLYSLSSV |
| | | VTVPSSSLGTKTYTCNVDHKPSNTKVDKRVE |
| | | SKYGPPCPPCPAPEFLGGPSVFLFPPKPKDT |
| | | LMISRTPEVTCVVVDVSQEDPEVQFNWYVDG |
| | | VEVHNAKTKPREEQFNSTYRVVSVLTVLHQD |
| | | WLNGKEYKCKVSNKGLPSSIEKTISKAKGQP |
| | | REPQVYTLPPSQEEMTKNQVSLTCLVKGFYP |
| | | SDIAVEWESNGQPENNYKTTPPVLDSDGSFF |
| | | LYSRLTVDKSRWQEGNVFSCSVMHEALHNHY |
| | | TQKSLSLSLG |
| SEQ ID | DNA | CAGGTGCAGCTGGTGCAGTCAGGCGCCGAAG |
| NO: 809 | heavy | TGAAGAAACCCGGCTCTAGCGTGAAAGTTTC |
| | chain | TTGTAAAGCTAGTGGCTACACCTTCACTAGC |
| | | TATAATATGCACTGGGTTCGCCAGGCCCCAG |
| | | GGCAAGGCCTCGAGTGGATGGGCGATATCTA |
| | | CCCCGGGAACGGCGACACTAGTTATAATCAG |
| | | AAGTTTAAGGGTAGAGTCACTATCACCGCCG |
| | | ATAAGTCTACTAGCACCGTCTATATGGAACT |
| | | GAGTTCCCTGAGGTCTGAGGACACCGCCGTC |
| | | TACTACTGCGCTAGAGTGGGCGGAGCCTTCC |
| | | CTATGGACTACTGGGGTCAAGGCACTACCGT |
| | | GACCGTGTCTAGCGCTAGCACTAAGGGCCCG |
| | | TCCGTGTTCCCCCTGGCACCTTGTAGCCGGA |
| | | GCACTAGCGAATCCACCGCTGCCCTCGGCTG |
| | | CCTGGTCAAGGATTACTTCCCGGAGCCCGTG |
| | | ACCGTGTCCTGGAACAGCGGAGCCCTGACCT |
| | | CCGGAGTGCACACCTTCCCCGCTGTGCTGCA |
| | | GAGCTCCGGGCTGTACTCGCTGTCGTCGGTG |
| | | GTCACGGTGCCTTCATCTAGCCTGGGTACCA |
| | | AGACCTACACTTGCAACGTGGACCACAAGCC |
| | | TTCCAACACTAAGGTGGACAAGCGCGTCGAA |
| | | TCGAAGTACGGCCCACCGTGCCCGCCTTGTC |
| | | CCGCGCCGGAGTTCCTCGGCGGTCCCTCGGT |
| | | CTTTCTGTTCCCACCGAAGCCCAAGGACACT |
| | | TTGATGATTTCCCGCACCCCTGAAGTGACAT |
| | | GCGTGGTCGTGGACGTGTCACAGGAAGATCC |
| | | GGAGGTGCAGTTCAATTGGTACGTGGATGGC |
| | | GTCGAGGTGCACAACGCCAAAACCAAGCCGA |
| | | GGGAGGAGCAGTTCAACTCCACTTACCGCGT |
| | | CGTGTCCGTGCTGACGGTGCTGCATCAGGAC |
| | | TGGCTGAACGGGAAGGAGTACAAGTGCAAAG |
| | | TGTCCAACAAGGGACTTCCTAGCTCAATCGA |
| | | AAAGACCATCTCGAAAGCCAAGGGACAGCCC |
| | | CGGGAACCCCAAGTGTATACCCTGCCACCGA |
| | | GCCAGGAAGAAATGACTAAGAACCAAGTCTC |
| | | ATTGACTTGCCTTGTGAAGGGCTTCTACCCA |
| | | TCGGATATCGCCGTGGAATGGGAGTCCAACG |
| | | GCCAGCCGGAAAACAACTACAAGACCACCCC |
| | | TCCGGTGCTGGACTCAGACGGATCCTTCTTC |
| | | CTCTACTCGCGGCTGACCGTGGATAAGAGCA |
| | | GATGGCAGGAGGGAAATGTGTTCAGCTGTTC |
| | | TGTGATGCATGAAGCCCTGCACAACCACTAC |
| | | ACTCAGAAGTCCCTGTCCCTCTCCCTGGGA |
| SEQ ID | LCDR1 | RASESVEYYGTSLMQ |
| NO: 810 | | |
| (Kabat) | | |
| SEQ ID | LCDR2 | AASNVES |
| NO: 811 | | |
| (Kabat) | | |
| SEQ ID | LCDR3 | QQSRKDPST |
| NO: 812 | | |
| (Kabat) | | |
| SEQ ID | LCDR1 | SESVEYYGTSL |
| NO: 813 | | |
| (Chothia) | | |
| SEQ ID | LCDR2 | AAS |
| NO: 814 | | |
| (Chothia) | | |
| SEQ ID | LCDR3 | SRKDPS |
| NO: 815 | | |
| (Chothia) | | |
| SEQ ID | VL | AIQLTQSPSSLSASVGDRVTITCRASESVEYY |
| NO: 816 | | GTSLMQWYQQKPGKAPKLLIYAASNVESGVPS |
| | | RFSGSGSGTDFTLTISSLQPEDFATYFCQQSR |
| | | KDPSTFGGGTKV |
| SEQ ID | DNA VL | EIKGCTATTCAGCTGACTCAGTCACCTAGTAG |
| NO: 817 | | CCTGAGCGCTAGTGTGGGCGATAGAGTGACTA |
| | | TCACCTGTAGAGCTAGTGAATCAGTCGAGTAC |
| | | TACGGCACTAGCCTGATGCAGTGGTATCAGCA |
| | | GAAGCCCGGGAAAGCCCCTAAGCTGCTGATCT |
| | | ACGCCGCCTCTAACGTGGAATCAGGCGTGCCC |
| | | TCTAGGTTTAGCGGTAGCGGTAGTGGCACCGA |
| | | CTTCACCCTGACTATCTCTAGCCTGCAGCCCG |
| | | AGGACTTCGCTACCTACTTCTGTCAGCAGTCT |
| | | AGGAAGGACCCTAGCACCTTCGGCGGAGGCAC |
| | | TAAG |
| SEQ ID | Light | GTCGAGATTAAGAIQLTQSPSSLSASVGDRVT |
| NO: 818 | chain | ITCRASESVEYYGTSLMQWYQQKPGKAPKLLI |
| | | YAASNVESGVPSRFSGSGSGTDFTLTISSLQP |
| | | EDFATYFCQQSRKDPSTFGGGTKVEIKRTVAA |
| | | PSVFIFPPSDEQLKSGTASVVCLLNNFYPREA |
| | | KVQWKVDNALQSGNSQESVTEQDSKDSTYSLS |
| | | STLTLSKADYEKHKVYACEVTHQG |
| SEQ ID | DNA | LSSPVTKSFNRGECGCTATTCAGCTGACTCAG |
| NO: 819 | light | TCACCTAGTAGCCTGAGCGCTAGTGTGGGCGA |
| | chain | TAGAGTGACTATCACCTGTAGAGCTAGTGAAT |
| | | CAGTCGAGTACTACGGCACTAGCCTGATGCAG |
| | | TGGTATCAGCAGAAGCCCGGGAAAGCCCCTAA |
| | | GCTGCTGATCTACGCCGCCTGCCCTCTAGGTT |
| | | TAGCGGTAGCGGTAGTGGCACCGACTAACGTG |
| | | GAATCAGGCGTCTTCACCCTGACTATCTCTAG |
| | | CCTGCAGCCCGAGGACTTCGCTACCTACTTCT |
| | | GTCAGCAGTCTAGGAAGGACCCTAGCACCTTC |
| | | GGCGGAGGCACTAAGGTCGAGATTAAGCGTAC |
| | | GGTGGCCGCTCCCAGCGTGTTCATCTTCCCCC |
| | | CCAGCGACGAGCAGCTGAAGAGCGGCACCGCC |
| | | AGCGTGGTGTGCCTGCTGAACAACTTCTACCC |
| | | CCGGGAGGCCAAGGTGCAGTGGAAGGTGGACA |
| | | ACGCCCTGCAGAGCGGCAACAGCCAGGAGAGC |
| | | GTCACCGAGCAGGACAGCAAGGACTCCACCTA |
| | | CAGCCTGAGCAGCACCCTGACCCTGAGCAAGG |
| | | CCGACTACGAGAAGCATAAGGTGTACGCCTGC |
| | | GAGGTGACCCACCAGGGCCTGTCCAGCCCCGT |
| | | GACCAAGAGCTTCAACAGGGGCGAGTGC |

ABTIM3-hum03

| | | |
|---|---|---|
| SEQ ID | HCDR1 | SYNMH |
| NO: 801 | | |
| (Kabat) | | |
| SEQ ID | HCDR2 | DIYPGQGDTSYNQKFKG |
| NO: 820 | | |
| (Kabat) | | |
| SEQ ID | HCDR3 | VGGAFPMDY |
| NO: 803 | | |
| (Kabat) | | |
| SEQ ID | HCDR1 | GYTFTSY |
| NO: 804 | | |
| (Chothia) | | |
| SEQ ID | HCDR2 | YPGQGD |
| NO: 821 | | |
| (Chothia) | | |
| SEQ ID | HCDR3 | VGGAFPMDY |
| NO: 803 | | |
| (Chothia) | | |
| SEQ ID | VH | QVQLVQSGAEVKKPGASVKVSCKASGYTFTSY |
| NO: 822 | | NMHWVRQAPGQGLEWIGDIYPGQGDTSYNQKF |
| | | KGRATMTADKSTSTVYMELSSLRSEDTAVYYC |
| | | ARVGGAFPMDYWGQGTLVTVSS |
| SEQ ID | DNA VH | CAGGTGCAGCTGGTGCAGTCAGGCGCCGAAGT |
| NO: 823 | | GAAGAAACCCGGCGCTAGTGTGAAAGTTAGCT |
| | | GTAAAGCTAGTGGCTATACTTTCACTTCTTAT |
| | | AATATGCACTGGGTCCGCCAGGCCCCAGGTCA |
| | | AGGCCTCGAGTGGATCGGCGATATCTACCCCG |
| | | GTCAAGGCGACACTTCCTATAATCAGAAGTTT |
| | | AAGGGTAGAGCTACTATGACCGCCGATAAGTC |
| | | TACTTCTACCGTCTATATGGAACTGAGTTCCC |
| | | TGAGGTCTGAGGACACCGCCGTCTACTACTGC |
| | | GCTAGAGTGGGCGGAGCCTTCCCAATGGACTA |
| | | CTGGGGTCAAGGCACCCTGGTCACCGTGTCTA |
| | | GC |
| SEQ ID | Heavy | QVQLVQSGAEVKKPGASVKVSCKASGYTFTSY |
| NO: 824 | chain | NMHWVRQAPGQGLEWIGDIYPGQGDTSYNQKF |
| | | KGRATMTADKSTSTVYMELSSLRSEDTAVYYC |
| | | ARVGGAFPMDYWGQGTLVTVSSASTKGPSVFP |
| | | LAPCSRSTSESTAALGCLVKDYFPEPVTVSWN |
| | | SGALTSGVHTFPAVLQSSGLYSLSSVVTVPSS |
| | | SLGTKTYTCNVDHKPSNTKVDKRVESKYGPPC |
| | | PPCPAPEFLGGPSVFLFPPKPKDTLMISRTPE |
| | | VTCVVVDVSQEDPEVQFNWYVDGVEVHNAKTK |
| | | PREEQFNSTYRVVSVLTVLHQDWLNGKEYKCK |
| | | VSNKGLPSSIEKTISKAKGQPREPQVYTLPPS |
| | | QEEMTKNQVSLTCLVKGFYPSDIAVEWESNGQ |
| | | PENNYKTTPPVLDSDGSFFLYSRLTVDKSRWQ |
| | | EGNVFSCSVMHEALHNHYTQKSLSLSLG |
| SEQ ID | DNA | CAGGTGCAGCTGGTGCAGTCAGGCGCCGAAGT |
| NO: 825 | heavy | GAAGAAACCCGGCGCTAGTGTGAAAGTTAGCT |
| | chain | GTAAAGCTAGTGGCTATACTTTCACTTCTTAT |
| | | AATATGCACTGGGTCCGCCAGGCCCCAGGTCA |
| | | AGGCCTCGAGTGGATCGGCGATATCTACCCCG |
| | | GTCAAGGCGACACTTCCTATAATCAGAAGTTT |
| | | AAGGGTAGAGCTACTATGACCGCCGATAAGTC |
| | | TACTTCTACCGTCTATATGGAACTGAGTTCCC |
| | | TGAGGTCTGAGGACACCGCCGTCTACTACTGC |
| | | GCTAGAGTGGGCGGAGCCTTCCCAATGGACTA |
| | | CTGGGGTCAAGGCACCCTGGTCACCGTGTCTA |
| | | GCGCTAGCTACTAAGGGCCCGCCGTGTTCCCC |
| | | CTGGCACCTTGTAGCCGGAGCACTAGCGAATC |
| | | CACCGCTGCCCTCGGCTGCCTGGTCAAGGATT |
| | | ACTTCCCGGAGCCCGTGACCGTGTCCTGGAAC |
| | | AGCGGAGCCCTGACCTCCGGAGTGCACACCTT |
| | | CCCCGCTGTGCTGCAGAGCTCCGGGCTGTACT |
| | | CGCTGTCGTCGGTGGTCACGGTGCCTTCATCT |
| | | AGCCTGGGTACCAAGACCTACACTTGCAACGT |
| | | GGACCACAAGCCTTCCAACACTAAGGTGGACA |
| | | AGCGCGTCGAATCGAAGTACGGCCCACCGTGC |
| | | CCGCCTTGTCCCGCGCCGGAGTTCCTCGGCGG |
| | | TCCCTCGGTCTTTCTGTTCCCACCGAAGCCCA |
| | | AGGACACTTTGATGATTTCCCGCACCCCTGAA |
| | | GTGACATGCGTGGTCGTGGACGTGTCACAGGA |
| | | AGATCCGGAGGTGCAGTTCAATTGGTACGTGG |
| | | ATGGCGTCGAGGTGCACAACGCCAAAACCAAG |
| | | CCGAGGGAGGAGCAGTTCAACTCCACTTACCG |
| | | CGTCGTGTCCGTGCTGACGGTGCTGCATCAGG |
| | | ACTGGCTGAACGGGAAGGAGTACAAGTGCAAA |
| | | GTGTCCAACAAGGGACTTCCTAGCTCAATCGA |
| | | AAAGACCATCTCGAAAGCCAAGGGACAGCCCC |
| | | GGGAACCCCAAGTGTATACCCTGCCACCGAGC |
| | | CAGGAAGAAATGACTAAGAACCAAGTCTCATT |
| | | GACTTGCCTTGTGAAGGGCTTCTACCCATCGG |
| | | ATATCGCCGTGGAATGGGAGTCCAACGGCCAG |
| | | CCGGAAAACAACTACAAGACCACCCCTCCGGT |
| | | GCTGGACTCAGACGGATCCTTCTTCCTCTACT |
| | | CGCGGCTGACCGTGGATAAGAGCAGATGGCAG |
| | | GAGGGAAATGTGTTCAGCTGTTCTGTGATGCA |
| | | TGAAGCCCTGCACAACCACTACACTCAGAAGT |
| | | CCCTGTCCCTCTCCCTGGGA |
| SEQ ID | LCDR1 | RASESVEYYGTSLMQ |
| NO: 810 | | |
| (Kabat) | | |
| SEQ ID | LCDR2 | AASNVES |
| NO: 811 | | |
| (Kabat) | | |
| SEQ ID | LCDR3 | QQSRKDPST |
| NO: 812 | | |
| (Kabat) | | |
| SEQ ID | LCDR1 | SESVEYYGTSL |
| NO: 813 | | |
| (Chothia) | | |
| SEQ ID | LCDR2 | AAS |
| NO: 814 | | |
| (Chothia) | | |
| SEQ ID | LCDR3 | SRKDPS |
| NO: 815 | | |
| (Chothia) | | |
| SEQ ID | VL | DIVLTQSPDSLAVSLGERATINCRASESVEYY |
| NO: 826 | | GTSLMQWYQQKPGQPPKLLIYAASNVESGVPD |
| | | RFSGSGSGTDFTLTISSLQAEDVAVYYCQQSR |
| | | KDPSTFGGGTKVEIK |
| SEQ ID | DNA VL | GATATCGTCCTGACTCAGTCACCCGATAGCCT |
| NO: 827 | | GGCCGTCAGCCTGGGCGAGCGGGCTACTATTA |
| | | ACTGTAGAGCTAGTGAATCAGTCGAGTACTAC |
| | | GGCACTAGCCTGATGCAGTGGTATCAGCAGAA |
| | | GCCCGGTCAACCCCCTAAGCTGCTGATCTACG |
| | | CCGCCTCTAACGTGGAATCAGGCGTGCCCGAT |
| | | AGGTTTAGCGGTAGCGGTAGTGGCACCGACTT |
| | | CACCCTGACTATTAGTAGCCTGCAGGCCGAGG |
| | | ACGTGGCCGTCTACTACTGTCAGCAGTCTAGG |
| | | AAGGACCCTAGCACCTTCGGCGGAGGCACTAA |
| | | GGTCGAGATTAAG |
| SEQ ID | Light | DIVLTQSPDSLAVSLGERATINCRASESVEYY |
| NO: 828 | chain | MQWYQQKPGQPPKLLIYAASNVESGVPDRFSG |
| | | GTSLSGSGTDFTLTISSLQAEDVAVYYCQQSR |
| | | KDPSTFGGGTKVEIKRTVAAPSVFIFPPSDEQ |
| | | LKSGTASVVCLLNNFYPREAKVQWKVDNALQS |
| | | GNSQESVTEQDSKDSTYSLSSTLTLSKADYEK |
| | | HKVYACEVTHQGLSSPVTKSFNRGEC |
| SEQ ID | DNA | GATATCGTCCTGACTCAGTCACCCGATAGCCT |
| NO: 829 | light | GGCCGTCAGCCTGGGCGAGCGGGCTACTATTA |
| | chain | ACTGTAGAGCTAGTGAATCAGTCGAGTACTAC |
| | | GGCACTAGCCTGATGCAGTGGTATCAGCAGAA |
| | | GCCCGGTCAACCCCCTAAGCTGCTGATCTACG |
| | | CCGCCTCTAACGTGGAATCAGGCGTGCCCGAT |
| | | AGGTTTAGCGGTAGCGGTAGTGGCACCGACTT |
| | | CACCCTGACTATTAGTAGCCTGCAGGCCGAGG |
| | | ACGTGGCCGTCTACTACTGTCAGCAGTCTAGG |
| | | AAGGACCCTAGCACCTTCGGCGGAGGCACTAA |
| | | GGTCGAGATTAAGCGTACGGTGGCCGCTCCCA |
| | | GCGTGTTCATCTTCCCCCCCAGCGACGAGCAG |
| | | CTGAAGAGCGGCACCGCCAGCGTGGTGTGCCT |
| | | GCTGAACAACTTCTACCCCCGGGAGGCCAAGG |
| | | TGCAGTGGAAGGTGGACAACGCCCTGCAGAGC |
| | | GGCAACAGCCAGGAGAGCGTCACCGAGCAGGA |
| | | CAGCAAGGACTCCACCTACAGCCTGAGCAGCA |
| | | CCCTGACCCTGAGCAAGGCCGACTACGAGAAG |
| | | CATAAGGTGTACGCCTGCGAGGTGACCCACCA |
| | | GGGCCTGTCCAGCCCCGTGACCAAGAGCTTCA |
| | | ACAGGGGCGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 18. APE5137, APE5121, and other anti-TIM-3 antibodies are disclosed in WO 2016/161270, incorporated by reference in its entirety.

In one embodiment, the anti-TIM-3 antibody molecule is the antibody clone F₃₈-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F₃₈-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 18
Amino acid sequences of other exemplary
anti-TIM-3 antibody molecules

APE5137

| SEQ | VH | EVQLLESGGGLVQPGGSLRLSCAAASGFTFSSYDMSWVRQAP |
|---|---|---|
| ID | | GKGLDWVSTISGGGTYTYYQDSVKGRFTISRDNSKNTLYLQM |
| NO: | | NSLRAEDTAVYYCASMDYWGQGTTVTVSSA |
| 830 | | |
| SEQ | VL | DIQMTQSPSSLSASVGDRVTITCRASQSIRRYLNWYHQKPGK |
| ID | | APKLLIYGASTLQSGVPSRFSGSGSGTDFTLTISSLQPEDFA |
| NO: | | VYYCQQSHSAPLTFGGGTKVEIKR |
| 831 | | |

APE5121

| | | |
|---|---|---|
| SEQ | VH | EVQVLESGGGLVQPGGSLRLYCVASGFTFSGSYAMSWVRQAP |
| ID | | GKGLEWVSAISGSGGSTYYADSVKGRFTISRDNSKNTLYLQM |
| NO: | | NSLRAEDTAVYYCAKKYYVGPADYWGQGTLVTVSSG |
| 832 | | |
| SEQ | VL | DIVMTQSPDSLAVSLGERATINCKSSQSVLYSSNNKNYLAWY |
| ID | | QHGQPPKLLIYWASTRESGVPDRFSGSGSGTDFTLTISSLQA |
| NO: | | EDVAVYYCQQYYSSPLTFGGGTKIEVK |
| 833 | | |

### Cytokines

In yet another embodiment, the present invention provides a method of treating cancer by administering to a subject in need thereof antibody conjugate of the present invention in combination with one or more cytokines, including but not limited to, interferon, IL-2, IL-15, IL-7, or IL21. In certain embodiments, antibody conjugate is administered in combination with an IL-15/IL-15Ra complex. In some embodiments, the IL-15/IL-15Ra complex is selected from NIZ985 (Novartis), ATL-803 (Altor) or CYP0150 (Cytune).

### Exemplary IL-15/IL-15Ra Complexes

In one embodiment, the cytokine is IL-15 complexed with a soluble form of IL-15 receptor alpha (IL-15Ra). The IL-15/IL-15Ra complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 922 in Table 21 or an amino acid sequence at least 85%, 90%, 95%, or 99% identical or higher to SEQ ID NO: 922, and the soluble form of human IL-15Ra comprises an amino acid sequence of SEQ ID NO:923 in Table 19, or an amino acid sequence at least 85%, 90%, 95%, or 99% identical or higher to SEQ ID NO: 923, as described in WO 2014/066527, incorporated by reference in its entirety. The molecules described herein can be made by vectors, host cells, and methods described in WO 2007084342, incorporated by reference in its entirety.

TABLE 19
Amino acid and nucleotide sequences
of exemplary IL-15/IL-15Ra complexes

NIZ985

| | | |
|---|---|---|
| SEQ ID NO: | Human | NWVNVISDLKKIEDLIQSMHIDATLYT |
| 922 | IL-15 | ESDVHPSCKVTAMKCFLLELQVISLES |
| | | GDASIHDTVENLIILANNSLSSNGNVT |
| | | ESGCKECEELEEKNIKEFLQSFVHIVQ |
| | | MFINTS |
| SEQ ID NO: | Human | ITCPPPMSVEHADIWVKSYSLYSRERY |
| 923 | Soluble | ICNSGFKRKAGTSSLTECVLNKATNVA |
| | IL-15Ra | HWTTPSLKCIRDPALVHQRPAPPSTVT |
| | | TAGVTPQPESLSPSGKEPAASSPSSNN |
| | | PTAATTAAIVPGSQLMPSKSSTGTTEI |
| | | SSHESSHGTPSQTTAKNWELTASASHQ |
| | | PPGVYPQG |

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is described in WO 2008/143794, incorporated by reference in its entirety. In one embodiment, the IL-15/IL-15Ra Fc fusion protein comprises the sequences as disclosed in Table 20.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is described in WO 2007/04606 and WO 2012/175222, incorporated by reference in their entirety. In one embodiment, the IL-15/IL-15Ra sushi domain fusion comprises the sequences as disclosed in Table 20.

TABLE 20
Amino acid sequences of other exemplary IL-15/IL-15Ra complexes
ALT-803

| | | |
|---|---|---|
| SEQ ID | IL-15N72D | NWVNVISDLKKIEDLIQSMHIDATLYTESDVHPSCKVTAMKCF |
| NO: 924 | | LLELQVISLESGDASIHDTVENLIILANDSLSSNGNVTESGCKE |
| | | CEELEEKNIKEFLQSFVHIVQMFINTS |
| SEQ ID | IL-15RaSu/ | ITCPPPMSVEHADIWVKSYSLYSRERYICNSGFKRKAGTSSLT |
| NO: 925 | Fc | ECVLNKATNVAHWTTPSLKCIREPKSCDKTHTCPPCPAPELL |
| | | GGPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSRDELTKN |
| | | QVSLTCLVKGFYPSDIAVEWESNGQPENNYKTTPPVLDSDGS |
| | | FFLYSKLTVDKSRWQQGNVFSCSVMHEALHNHYTQKSLSLS |
| | | PGK |

IL-15 / IL-15Ra sushi domain fusion (CYP0150)

| | | |
|---|---|---|
| SEQ ID | Human | IL-15 NWVNVISDLKKIEDLIQSMHIDATLYTESDVHPSCKVT |
| NO: 926 | | AMKCFLLELQVISLESGDASIHDTVENLIILANNSLSSNGNVTE |
| | | SGCKECEELEXKNIKEFLQSFVHIVQMFINTS |
| | | Where X is E or K |
| SEQ ID | Human IL- | ITCPPPMSVEHADIWVKSYSLYSRERYICNSGFKRKAGTSSLT |
| NO: 927 | 15Ra sushi | ECVLNKATNVAHWTTPSLKCIRDPALVHQRPAPP |
| | and hinge | |
| | domains | |

In yet another embodiment, the present invention provides a method of treating cancer by administering to a subject in need thereof antibody conjugate of the present invention in combination with one or more agonists of toll like receptors (TLRs, e.g., TLR7, TLR8, TLR9). In some embodiments, the antibody conjugate of the present invention can be used in combination with a TLR7 agonist or a TLR7 agonist conjugate.

In another embodiment, the present invention provides a method of treating cancer by administering to a subject in need thereof antibody conjugate of the present invention in combination with one or more angiogenesis inhibitors, e.g., Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TK1258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); or Aflibercept (Eylea®).

In another embodiment, the present invention provides a method of treating cancer by administering to a subject in need thereof antibody conjugate of the present invention in combination with one or more heat shock protein inhibitors, e.g., Tanespimycin (17-allylamino-17-demethoxygeldanamycin, also known as KOS-953 and 17-AAG, available from SIGMA, and described in U.S. Pat. No. 4,261,989); Retaspimycin (IP1504), Ganetespib (STA-9090); [6-Chloro-9-(4-methoxy-3,5-dimethylpyridin-2-ylmethyl)-9H-purin-2-yl]amine (BIIB021 or CNF2024, CAS 848695-25-0); trans-4-[[2-(Aminocarbonyl)-5-[4,5,6,7-tetrahydro-6,6-dimethyl-4-oxo-3-(trifluoromethyl)-1H-indazol-1-yl]phenyl]amino]cyclohexyl glycine ester (SNX5422 or PF04929113, CAS 908115-27-5); 5-[2,4-Dihydroxy-5-(1-methylethyl)phenyl]-N-ethyl-4-[4-(4-morpholinylmethyl)phenyl]-3-Isoxazolecarboxamide (AUY922, CAS 747412-49-3); or 17-Dimethylaminoethylamino-17-demethoxygeldanamycin (17-DMAG).

In another embodiment, the present invention provides a method of treating cancer by administering to a subject in need thereof antibody conjugate of the present invention in combination with one or more HDAC inhibitors or other epigenetic modifiers. Exemplary HDAC inhibitors include, but not limited to, Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-η-oxo-2-oxiraneoctanoyl-O-methyl-D-tyrosyl-L-isoleucyl-L-prolyl] (Cyl-1); Cyclo[(αS,2S)-α-amino-η-oxo-2-oxiraneoctanoyl-O-methyl-D-tyrosyl-L-isoleucyl-(2S)-2-piperidinecarbonyl] (Cyl-2); Cyclic[L-alanyl-D-alanyl-(2S)-η-oxo-L-α-aminooxiraneoctanoyl-D-prolyl] (HC-toxin); Cyclo[(αS,2S)-α-amino-η-oxo-2-oxiraneoctanoyl-D-phenylalanyl-L-leucyl-(2S)-2-piperidinecarbonyl] (WF-3161); Chlamydocin ((S)-Cyclic(2-methylalanyl-L-phenylalanyl-D-prolyl-η-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); Depudecin (4,5:8,9-dianhydro-1,2,6,7,11-pentadeoxy-D-threo-D-ido-Undeca-1,6-dienitol); 4-(Acetylamino)-N-(2-aminophenyl)-benzamide (also known as CI-994); N1-(2-Aminophenyl)-N8-phenyl-octanediamide (also known as BML-210); 4-(Dimethylamino)-N-(7-(hydroxyamino)-7-oxoheptyl)benzamide (also known as M344); (E)-3-(4-(((2-(1H-indol-3-yl)ethyl)(2-hydroxyethyl)amino)-methyl)phenyl)-N-hydroxyacrylamide; Panobinostat (Farydak®); Mocetinostat, and Belinostat (also known as PXD101, Beleodaq®, or (2E)-N-Hydroxy-3-[3-(phenylsulfamoyl)phenyl]prop-2-enamide), or chidamide (also known as CS055 or HBI-8000, (E)-N-(2-amino-5-fluorophenyl)-4-((3-(pyridin-3-yl)acrylamido)methyl)benzamide). Other epigenetic modifiers include but not limited to inhibitors of EZH2 (enhancer of zeste homolog 2), EED (embryonic ectoderm development), or LSD1 (lysine-specific histone demethylase 1A or KDM1A).

In yet another embodiment, the present invention provides a method of treating cancer by administering to a subject in need thereof antibody conjugate of the present invention in combination with one or more inhibitors of indoleamine-pyrrole 2,3-dioxygenase (IDO), for example, Indoximod (also known as NLG-8189), α-Cyclohexyl-5H-imidazo[5,1-a]isoindole-5-ethanol (also known as NLG919), or (4E)-4-[(3-Chloro-4-fluoroanilino)-nitrosomethylidene]-1,2,5-oxadiazol-3-amine (also known as INCB024360).

In yet another embodiment, the present invention provides a method of treating cancer by administering to a subject in need thereof antibody conjugate of the present invention in combination with one or more agents that control or treat cytokine release syndrome (CRS). Therapies for CRS include but not are limited to, IL-6 inhibitor or IL-6 receptor (IL-6R) inhibitors (e.g., tocilizumab or siltuximab), bazedoxifene, sgp130 blockers, vasoactive medications, corticosteroids, immunosuppressive agents, histamine H₂ receptor antagonists, anti-pyretics, analgesics (e.g., acetaminophen), and mechanical ventilation. Exemplary therapies for CRS are described in International Application WO2014011984, which is hereby incorporated by reference.

Tocilizumab is a humanized, immunoglobulin G1kappa anti-human IL-6R monoclonal antibody. Tocilizumab blocks binding of IL-6 to soluble and membrane bound IL-6 receptors (IL-6Rs) and thus inhibitos classical and trans-IL-6 signaling. In embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the CRS therapeutic is an inhibitor of IL-6 signalling, e.g., an inhibitor of IL-6 or IL-6 receptor. In one embodiment, the inhibitor is an anti-IL-6 antibody, e.g., an anti-IL-6 chimeric monoclonal antibody such as siltuximab. In other embodiments, the inhibitor comprises a soluble gp130 (sgp130) or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the inhibitor of IL-6 signalling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signalling comprises a small molecule such as CPSI-2364.

Exemplary vasoactive medications include but are not limited to angiotensin-11, endothelin-1, alpha adrenergic agonists, rostanoids, phosphodiesterase inhibitors, endothelin antagonists, inotropes (e.g., adrenaline, dobutamine, isoprenaline, ephedrine), vasopressors (e.g., noradrenaline, vasopressin, metaraminol, vasopressin, methylene blue), inodilators (e.g., milrinone, levosimendan), and dopamine.

Exemplary vasopressors include but are not limited to norepinephrine, dopamine, phenylephrine, epinephrine, and vasopressin. In some embodiments, a high-dose vasopressor includes one or more of the following: norpepinephrine monotherapy at ≥20 ug/min, dopamine monotherapy at ≥10 ug/kg/min, phenylephrine monotherapy at ≥200 ug/min, and/or epinephrine monotherapy at ≥10 ug/min. In some embodiments, if the subject is on vasopressin, a high-dose vasopressor includes vasopressin+norepinephrine equivalent of ≥10 ug/min, where the norepinephrine equivalent dose=[norepinephrine (ug/min)]+[dopamine (ug/kg/min)/2]+[epinephrine (ug/min)]+[phenylephrine (ug/min)/10]. In some embodiments, if the subject is on combination vasopressors (not vasopressin), a high-dose vasopressor includes norepinephrine equivalent of ≥20 ug/min, where the norepinephrine equivalent dose=[norepinephrine (ug/min)]+[dopamine (ug/kg/min)/2]+[epinephrine (ug/min)]+[phenylephrine (ug/min)/10]. See e.g., Id.

In some embodiments, a low-dose vasopressor is a vasopressor administered at a dose less than one or more of the doses listed above for high-dose vasopressors.

Exemplary corticosteroids include but are not limited to dexamethasone, hydrocortisone, and methylprednisolone. In embodiments, a dose of dexamethasone of 0.5 mg/kg is used. In embodiments, a maximum dose of dexamethasone of 10 mg/dose is used. In embodiments, a dose of methylprednisolone of 2 mg/kg/day is used.

Exemplary immunosuppressive agents include but are not limited to an inhibitor of TNFα or an inhibitor of IL-1. In embodiments, an inhibitor of TNFα comprises an anti-TNFα antibody, e.g., monoclonal antibody, e.g., infliximab. In embodiments, an inhibitor of TNFα comprises a soluble TNFα receptor (e.g., etanercept). In embodiments, an IL-1 or IL-1R inhibitor comprises anakinra.

Exemplary histamine H₂ receptor antagonists include but are not limited to cimetidine (Tagamet®), ranitidine (Zantac®), famotidine (Pepcid®) and nizatidine (Axid®).

Exemplary anti-pyretic and analgesic includes but is not limited to acetaminophen (Tylenol®), ibuprofen, and aspirin.

In some embodiments, the present invention provides a method of treating cancer by administering to a subject in need thereof antibody conjugate of the present invention in combination with two or more of any of the above described inhibitors, activators, immunomodulators, agonists, or modifiers. For example, the antibody conjugate of the present invention can be used in combination with one or more checkpoint inhibitors and/or one or more immune activators.

In addition to the above therapeutic regimes, the patient may be subjected to surgical removal of cancer cells and/or radiation therapy.

### Pharmaceutical Compositions

To prepare pharmaceutical or sterile compositions including one or more antibody conjugates described herein, provided antibody conjugate can be mixed with a pharmaceutically acceptable carrier or excipient.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, NY, 1993; Lieberman, et al. (eds.), Pharmaceutical Dosage Forms: Tablets, Marcel Dekker, NY, 1990; Lieberman, et al. (eds.) Pharmaceutical Dosage Forms: Disperse Systems, Marcel Dekker, NY, 1990; Weiner and Kotkoskie, Excipient Toxicity and Safety, Marcel Dekker, Inc., New York, N.Y., 2000).

In some embodiments, the pharmaceutical composition comprising the antibody conjugate of the present invention is a lyophilisate preparation. In certain embodiments a pharmaceutical composition comprising the antibody conjugate is a lyophilisate in a vial containing an antibody conjugate, histidine, sucrose, and polysorbate 20. In certain embodiments the pharmaceutical composition comprising the antibody conjugate is a lyophilisate in a vial containing an antibody conjugate, sodium succinate, and polysorbate 20. In certain embodiments the pharmaceutical composition comprising the antibody conjugate is a lyophilisate in a vial containing an antibody conjugate, trehalose, citrate, and polysorbate 8. The lyophilisate can be reconstituted, e.g., with water, saline, for injection. In a specific embodiment, the solution comprises the antibody conjugate, histidine, sucrose, and polysorbate 20 at a pH of about 5.0. In another specific embodiment the solution comprises the antibody conjugate, sodium succinate, and polysorbate 20. In another specific embodiment, the solution comprises the antibody conjugate, trehalose dehydrate, citrate dehydrate, citric acid, and polysorbate 8 at a pH of about 6.6. For intravenous administration, the obtained solution will usually be further diluted into a carrier solution.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, U K, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising the antibody conjugate of the invention can be provided by continuous infusion, or by doses at intervals of, e.g., one day, one week, or 1-7 times per week, once every other week, once every three weeks, once every four weeks, once every five weeks, once every six weeks, once every seven weeks, or once very eight weeks. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects.

For the antibody conjugates of the invention, the dosage administered to a patient may be 0.0001 mg/kg to 100 mg/kg of the patient's body weight. The dosage may be between 0.001 mg/kg and 50 mg/kg, 0.005 mg/kg and 20 mg/kg, 0.01 mg/kg and 20 mg/kg, 0.02 mg/kg and 10 mg/kg, 0.05 and 5 mg/kg, 0.1 mg/kg and 10 mg/kg, 0.1 mg/kg and 8 mg/kg, 0.1 mg/kg and 5 mg/kg, 0.1 mg/kg and 2 mg/kg, 0.1 mg/kg and 1 mg/kg of the patient's body weight. The dosage of the antibody conjugate may be calculated using the patient's weight in kilograms (kg) multiplied by the dose to be administered in mg/kg.

Doses of the antibody conjugates the invention may be repeated and the administrations may be separated by less than 1 day, at least 1 day, 2 days, 3 days, 5 days, 10 days, 15 days, 30 days, 45 days, 2 months, 75 days, 3 months, 4 months, 5 months, or at least 6 months. In some embodiments, an antibody conjugate of the invention is administered twice weekly, once weekly, once every two weeks, once every three weeks, once every four weeks, or less frequently. In a specific embodiment, doses of the antibody conjugates of the invention are repeated every 2 weeks.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, U K, 2001).

The route of administration may be by, e.g., topical or cutaneous application, injection or infusion by subcutaneous, intravenous, intraperitoneal, intracerebral, intramuscular, intraocular, intraarterial, intracerebrospinal, intralesional administration, or by sustained release systems or an implant (see, e.g., Sidman et al., Biopolymers 22:547-556, 1983; Langer et al., J. Biomed. Mater. Res. 15:167-277, 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). Where necessary, the composition may also include a solubilizing agent or a local anesthetic such as lidocaine to ease pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

Examples of such additional ingredients are well-known in the art.

Methods for co-administration or treatment with a second therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the antibody conjugates of the invention may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibody conjugates of the invention. The two or more therapies may be administered within one same patient visit.

In certain embodiments, the antibody conjugates of the invention can be formulated to ensure proper distribution in vivo. Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant Protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The invention provides protocols for the administration of pharmaceutical composition comprising antibody conjugates of the invention alone or in combination with other therapies to a subject in need thereof. The therapies (e.g., prophylactic or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the invention can be administered to a subject concurrently.

The term “concurrently” is not limited to the administration of therapies (e.g., prophylactic or therapeutic agents) at exactly the same time, but rather it is meant that a pharmaceutical composition comprising antibodies or fragments thereof the invention are administered to a subject in a sequence and within a time interval such that the antibodies or antibody conjugates of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 5 minutes apart, less than 15 minutes apart, less than 30 minutes apart, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered within the same patient visit.

Prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The invention is further described in the following examples, which are not intended to limit the scope of the invention described in the claims.

### Example 1: Synthesis of Linker Intermediates

### Example 1-1: Synthesis of 5,5,9,12,15,15-hexamethyl-8,13-dioxo-14-oxa-3,4-dithia-9,12-diazahexadecyl carbonochloridate (LI-1)

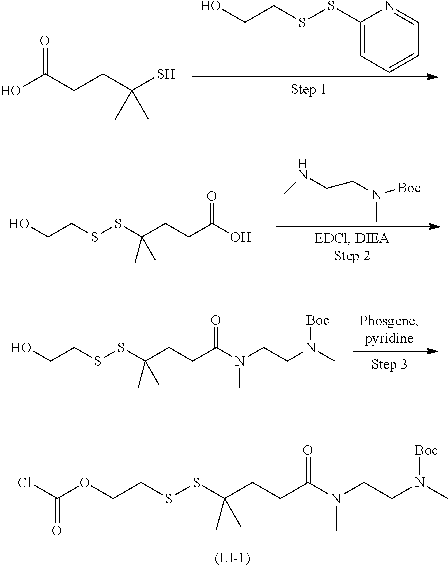

Step 1: Acetic acid (0.025 ml, 1.3 mmol) was added to a solution of 4-mercapto-4-methylpentanoic acid (250 mg, 1.69 mmol) and 2-(pyridin-2-yldisulfanyl)ethanol (380 mg, 2.02 mmol) in MeOH (15 mL) and the mixture was heated at 45° C. for 5 days and then concentrated and purified by ISCO using 15 g C18 column, eluted with 5-40% acetonitrile (ACN) in water with 0.05% TFA. The fractions containing the desired product were concentrated to give 4-((2-hydroxyethyl)disulfanyl)-4-methylpentanoic acid (220 mg, 58.1% yield). LCMS M+23=247.1, tr=0.768 min. 1H NMR (500 MHz, Chloroform-d) δ 3.86 (t, J=5.8 Hz, 1H), 2.84 (t, J=5.8 Hz, 2H), 2.49-2.37 (m, 2H), 2.00-1.86 (m, 2H), 1.29 (s, 6H).

Step 2: DIEA (0.082 ml, 0.47 mmol) and tert-butyl methyl(2-(methylamino)ethyl)carbamate (44 mg, 0.23 mmol) were added to a solution of 4-((2-hydroxyethyl)disulfanyl)-4-methylpentanoic acid (35 mg, 0.16 mmol) in dichloromethane (DCM) (5 ml), followed by the addition of N1-((ethylimino)methylene)-N3,N3-dimethylpropane-1,3-diamine hydrochloride (EDCl) (45 mg, 0.23 mmol). The mixture was stirred at room temperature for 16 hours, then quenched with water, extracted with DCM, dried, concentrated and purified by ISCO using 15 g C18 column, eluted with ACN-water containing 0.05% TFA to obtain tert-butyl (2-(4-((2-hydroxyethyl)disulfanyl)-N,4-dimethylpentanamido)ethyl)(methyl)carbamate (34 mg, 50% yield). LCMS M+1=395.2, tr=1.044 min. ¹H NMR (500 MHz, Chloroform-d) δ 3.84 (t, J=6.0 Hz, 2H), 3.49 (s, 2H), 3.35 (t, J=6.1 Hz, 2H), 3.03 (s, 2H), 2.94 (s, 1H), 2.89-2.78 (m, 5H), 2.38 (d, J=7.3 Hz, 2H), 2.01-1.90 (m, 2H), 1.83 (s, 3H), 1.44 (s, 9H), 1.30 (s, 6H).

Step 3: Pyridine (0.010 ml, 0.12 mmol) was added to a solution of tert-butyl (2-(4-((2-hydroxyethyl)disulfanyl)-N,4-dimethylpentanamido)ethyl)(methyl)carbamate (27 mg, 0.068 mmol) in DCM (4 ml) at 00° C. followed by addition of a 20% phosgene solution in toluene (0.3 ml). The reaction was stirred for 15 mins and then concentrated to give 5,5,9,12,15,15-hexamethyl-8,13-dioxo-14-oxa-3,4-dithia-9,12-diazahexadecyl carbonochloridate (LI-1) which was immediately used without purification.

### Example 1-2: Synthesis of 18-(2,5-dioxo-2,5-dihydro-1H-pyrrol-1-yl)-5,5,9,12-tetramethyl-8,13-dioxo-16-oxa-3,4-dithia-9,12-diazaoctadecyl (4-nitrophenyl) carbonate (LI-2)

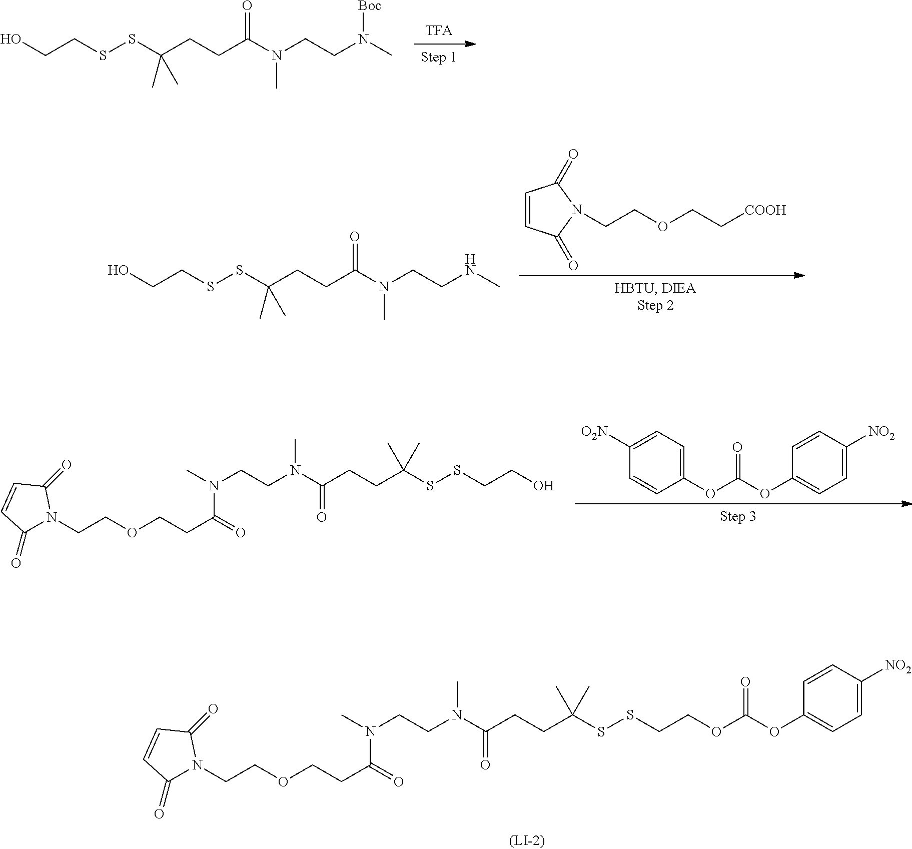 [SMILES: CN(CCN(C)C(=O)OC(C)(C)C)C(=O)CCC(C)(C)SSCCO ; CNCCN(C)C(=O)CCC(C)(C)SSCCO ; O=C(O)CCOCCN1C(=O)C=CC1=O ; CN(CCN(C)C(=O)CCC(C)(C)SSCCO)C(=O)CCOCCN1C(=O)C=CC1=O ; O=C(Oc1ccc([N+](=O)[O-])cc1)Oc1ccc([N+](=O)[O-])cc1 ; CN(CCN(C)C(=O)CCC(C)(C)SSCCOC(=O)Oc1ccc([N+](=O)[O-])cc1)C(=O)CCOCCN1C(=O)C=CC1=O]

Step 1: Trifluoroacetic acid (TFA) (1 ml) was added to a flask containing tert-butyl (2-(4-((2-hydroxyethyl)disulfanyl)-N,4-dimethylpentanamido)ethyl)(methyl)carbamate (34 mg, 0.086 mmol) and the mixture was immediately concentrated to give 4-((2-hydroxyethyl)disulfanyl)-N,4-dimethyl-N-(2-(methylamino)ethyl)pentanamide as a TFA salt. LCMS M+1=295.3, tr=0.619 min.

Step 2: N,N-diisopropyl ethylamine (DIEA) (0.075 ml, 0.431 mmol) was added to a solution of 3-(2-(2,5-dioxo-2,5-dihydro-1H-pyrrol-1-yl)ethoxy)propanoic acid (Mal-PEG1-Acid) (18.4 mg, 0.086 mmol) in DMF (2 ml), followed by the addition of 3-[Bis(dimethylamino)methyliumyl]-3H-benzotriazol-1-oxide hexafluorophosphate (HBTU) (33 mg, 0.086 mmol). The mixture was stirred at room temperature for 5 mins and then added dropwise to a solution of 4-((2-hydroxyethyl)disulfanyl)-N,4-dimethyl-N-(2-(methylamino)ethyl)pentanamide TFA salt (35 mg, 0.086 mmol) in N,N-dimethyl formamide (DMF) (1 ml). The mixture was then stirred at room temperature for 2 hours and then purified by mass-triggered reverse phase HPLC using a C18 column, eluted with 10-40% acetonitrile-H₂O containing 0.05% TFA. Fractions containing desired product were concentrated to obtain N-(2-(3-(2-(2,5-dioxo-2,5-dihydro-1H-pyrrol-1-yl)ethoxy)-N-methylpropanamido)ethyl)-4-((2-hydroxyethyl)disulfanyl)-N,4-dimethylpentanamide (40.1 mg, 90% yield). LCMS M+1=490.3 tr=0.841 min.

Step 3: To a solution of N-(2-(3-(2-(2,5-dioxo-2,5-dihydro-1H-pyrrol-1-yl)ethoxy)-N-methylpropanamido)ethyl)-4-((2-hydroxyethyl)disulfanyl)-N,4-dimethylpentanamide (40.1 mg, 0.082 mmol) obtained in step 2 in DCM (3 ml) was added bis(4-nitrophenyl) carbonate (125 mg, 0.409 mmol) and then DIEA (0.043 mL, 0.246 mmol). It was stirred at room temperature for 4 days and the reaction was complete to form the desired product. It was concentrated and the residue was dissolved in ACN and purified by ISCO using 50 g C18 column, eluted with 25-75% ACN in water with 0.035% TFA. Fractions containing the desired product were combined and lyophilized to give 18-(2,5-dioxo-2,5-dihydro-1H-pyrrol-1-yl)-5,5,9,12-tetramethyl-8,13-dioxo-16-oxa-3,4-dithia-9,12-diazaoctadecyl (4-nitrophenyl) carbonate (LI-2) (44 mg, 73% yield). LCMS M+1=655.2, tr=1.177 min. It is contaminated by a small amount of bis (4-nitrophenyl) carbonate and hydrolyzed alcohol by-product.

### Example 1-3: Synthesis of 4-((S)-2-((S)-2-(3-(2,5-dioxo-2,5-dihydro-1H-pyrrol-1-yl)propanamido)-3-methylbutanamido)-5-ureidopentanamido)benzyl (4-nitrophenyl) carbonate (LI-3)

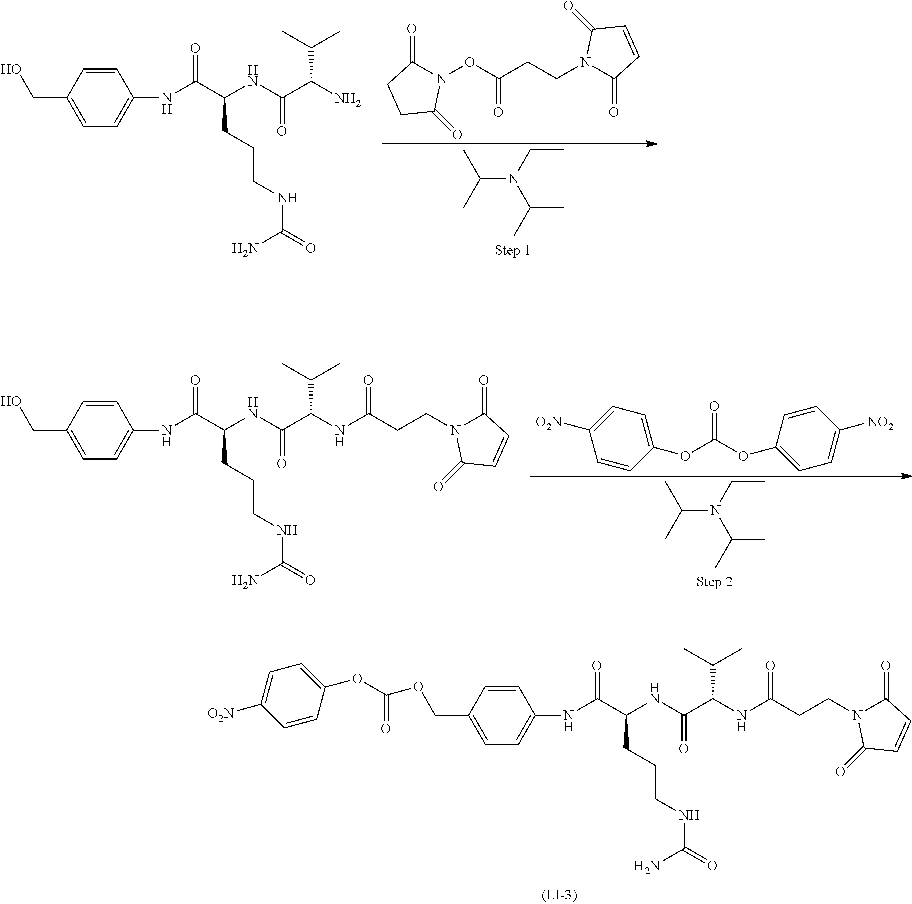

Step 1: (S)-2-((S)-2-amino-3-methylbutanamido)-N-(4-(hydroxymethyl)phenyl)-5-ureidopentanamide (valcit-pab-OH) (100 mg, 0.264 mmol) (purchased from Levena Biopharma, San Diego) was added to 2,5-dioxopyrrolidin-1-yl 3-(2,5-dioxo-2,5-dihydro-1H-pyrrol-1-yl)propanoate (77 mg, 0.29 mmol) in DMF (5 ml) at room temperature, followed by the addition of DIEA (70 mg, 0.54 mmol). The mixture was stirred at room temperature for 2 hrs, concentrated and then purified by ISCO using 50 g C18 aq column, eluted with 10-25% ACN-water with 0.05% TFA. Fractions containing (S)-2-((S)-2-(3-(2,5-dioxo-2,5-dihydro-1H-pyrrol-1-yl)propanamido)-3-methylbutanamido)-N-(4-(hydroxymethyl)phenyl)-5-ureidopentanamide (MP-valcit-pab-OH) were combined and concentrated (79.8 mg, 0.150 mmol, 57.1% yield). LCMS M+1=531.3, tr=0.687 min.

Step 2: A solution of MP-valcit-pab-OH (33 mg, 0.062 mmol), bis(4-nitrophenyl) carbonate (189 mg, 0.622 mmol) and DIEA (0.033 mL, 0.19 mmol) in DMF-DCM (1:4, 5 ml) was stirred at room temperature for 1 week, then concentrated and purified by silica gel column, eluted with MeOH:DCM (2% to 10%). Fractions containing the desired compound were combined and concentrated to give 4-((S)-2-((S)-2-(3-(2,5-dioxo-2,5-dihydro-1H-pyrrol-1-yl)propanamido)-3-methylbutanamido)-5-ureidopentanamido)benzyl (4-nitrophenyl) carbonate (LI-3) (20 mg, 0.029 mmol, 46% yield). LCMS M+1=696.3, tr=1.039 min.

### Example 1-4: Synthesis of (S)-4-(2-(3-(2,5-dioxo-2,5-dihydro-1H-pyrrol-1-yl)propanamido)-3-phenylpropanamido)benzyl (4-nitrophenyl) carbonate (LI-4)

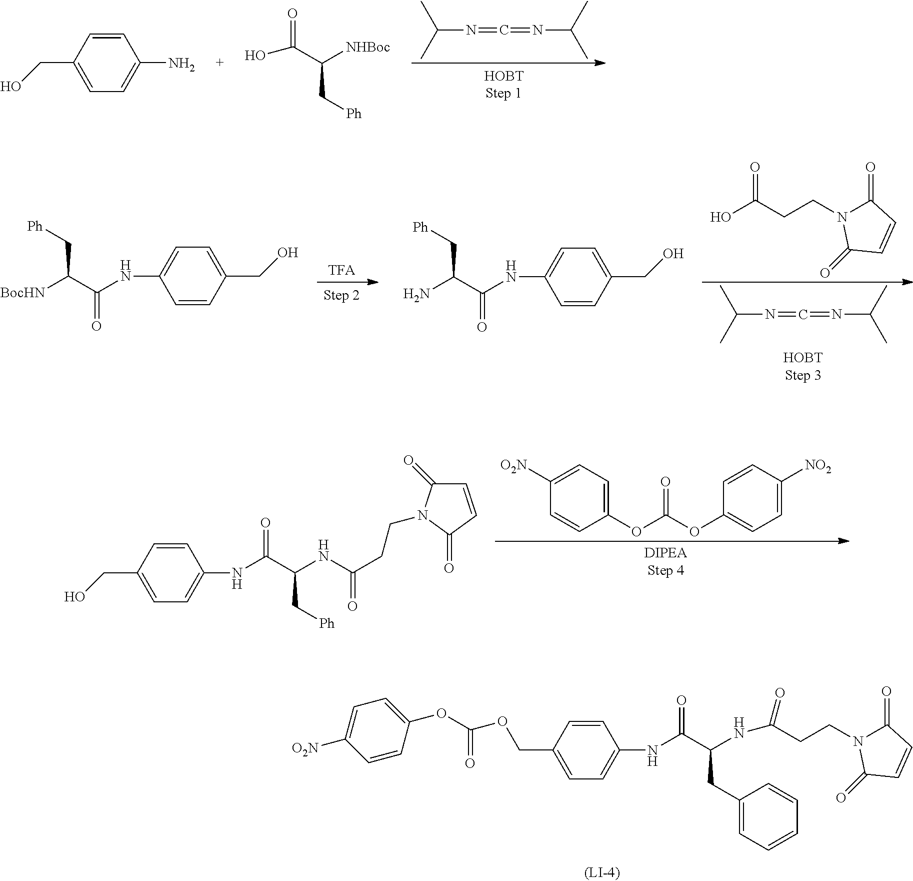

Step 1: N-Hydroxybenzotriazole (HOBT) (509 mg, 3.77 mmol) and DMF (6 ml) was added to a solution of BocPhe-OH (500 mg, 1.89 mmol) and (4-aminophenyl)methanol (464 mg, 3.77 mmol) in DCM (30 ml), followed by the addition of diisopropylcarbodiimide (476 mg, 3.77 mmol). The mixture was stirred at room temperature for 16 hours, concentrated to remove DCM and then purified by silica gel column eluted with 10% MeOH in DCM to give tert-butyl (S)-(1-((4-(hydroxymethyl)phenyl)amino)-1-oxo-3-phenylpropan-2-yl)carbamate (1.12 g, 97% yield). LCMS M+1=275.2. tr=0.561 min. 1H NMR (500 MHz, Chloroform-d) δ 7.99 (s, 1H), 7.88 (d, J=7.1 Hz, 1H), 7.39-7.18 (m, 9H), 5.17 (s, 1H), 4.60 (s, 2H), 4.46 (s, 1H), 3.12 (d, J=6.9 Hz, 2H), 1.40 (s, 9H).

Step 2: TFA (5 ml) and DCM (1 ml) were added to tert-butyl (S)-(1-((4-(hydroxymethyl)phenyl)amino)-1-oxo-3-phenylpropan-2-yl)carbamate (1.12 g, 1.82 mmol) and the mixture was concentrated immediately. The solid was then dissolved in MeOH-DCM (5%) and extracted from 2M Na₂CO₃ aqueous solution, dried and concentrated to obtain (S)-2-amino-N-(4-(hydroxymethyl)phenyl)-3-phenylpropanamide (Phe-pab-OH), which was used in the next step without further purification. LCMS M+1=271.3 tr=0.618 min.

Step 3: HOBT (200 mg, 1.48 mmol) was added to a solution of Phe-pab-OH (400 mg, 1.48 mmol) and 3-(2,5-dioxo-2,5-dihydro-1H-pyrrol-1-yl)propanoic acid (250 mg, 1.480 mmol) in DCM-DMF (5:1, 24 ml), followed by the addition of diisopropylcarbodiimide (187 mg, 1.48 mmol). The mixture was stirred at room temperature for 16 hours, concentrated and purified by silica gel column, eluted with 5% MeOH in DCM. Fractions containing the desired product were combined and concentrated. The mixture was further purified by reverse phase ISCO using 50 g C18 aq column, eluted with 10-50% acetonitrile-H2O containing 0.05% TFA. Fractions containing the desired product were concentrated to obtain (S)-2-(3-(2,5-dioxo-2,5-dihydro-1H-pyrrol-1-yl)propanamido)-N-(4-(hydroxymethyl)phenyl)-3-phenylpropanamide (MP-Phe-pab-OH) (0.214 g, 32.6% yield) as free base. LCMS M+1=422.2, tr=0.851 min. 1H NMR (500 MHz, Acetonitrile-d3) δ 8.40 (s, 1H), 7.45 (d, J=8.5 Hz, 2H), 7.25 (ddd, J=20.2, 7.7, 3.3 Hz, 7H), 6.80 (d, J=7.8 Hz, 1H), 6.70 (s, 2H), 4.62 (td, J=8.0, 6.2 Hz, 1H), 4.51 (s, 2H), 3.64 (t, J=7.0 Hz, 2H), 3.13 (dd, J=13.9, 6.2 Hz, 1H), 2.93 (dd, J=13.9, 8.1 Hz, 1H), 2.54-2.31 (m, 2H).

Step 4: A solution of (S)-2-(3-(2,5-dioxo-2,5-dihydro-1H-pyrrol-1-yl)propanamido)-N-(4-(hydroxymethyl)phenyl)-3-phenylpropanamide (MP-Phe-pab-OH) (89.3 mg, 0.212 mmol), bis(4-nitrophenyl) carbonate (645 mg, 2.119 mmol) and DIEA (0.111 mL, 0.636 mmol) was stirred at room temperature for 2 days, then concentrated and purified by silica gel column, eluted with 2-6% MeOH:DCM. Fractions containing the desired product were collected and concentrated to give (S)-4-(2-(3-(2,5-dioxo-2,5-dihydro-1H-pyrrol-1-yl)propanamido)-3-phenylpropanamido)benzyl (4-nitrophenyl) carbonate (LI-4) (116 mg, 89% yield). LCMS M+1=587.2, tr=1.268 min. 1H NMR (500 MHz, DMSO-d6) δ 10.21 (s, 1H), 8.46 (d, J=8.1 Hz, 1H), 8.40-8.23 (m, 2H), 7.68-7.56 (m, 4H), 7.45 (d, J=8.6 Hz, 2H), 7.30 (d, J=4.4 Hz, 4H), 7.01 (s, 2H), 5.28 (s, 2H), 4.68 (dt, J=8.7, 4.4 Hz, 1H), 3.63-3.48 (m, 2H), 3.36 (s, 4H), 3.05 (dd, J=13.7, 5.5 Hz, 1H), 2.92-2.83 (m, 2H), 2.44-2.34 (m, 2H).

### Example 1-5: Synthesis of 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (4-nitrophenyl) carbonate (LI-5)

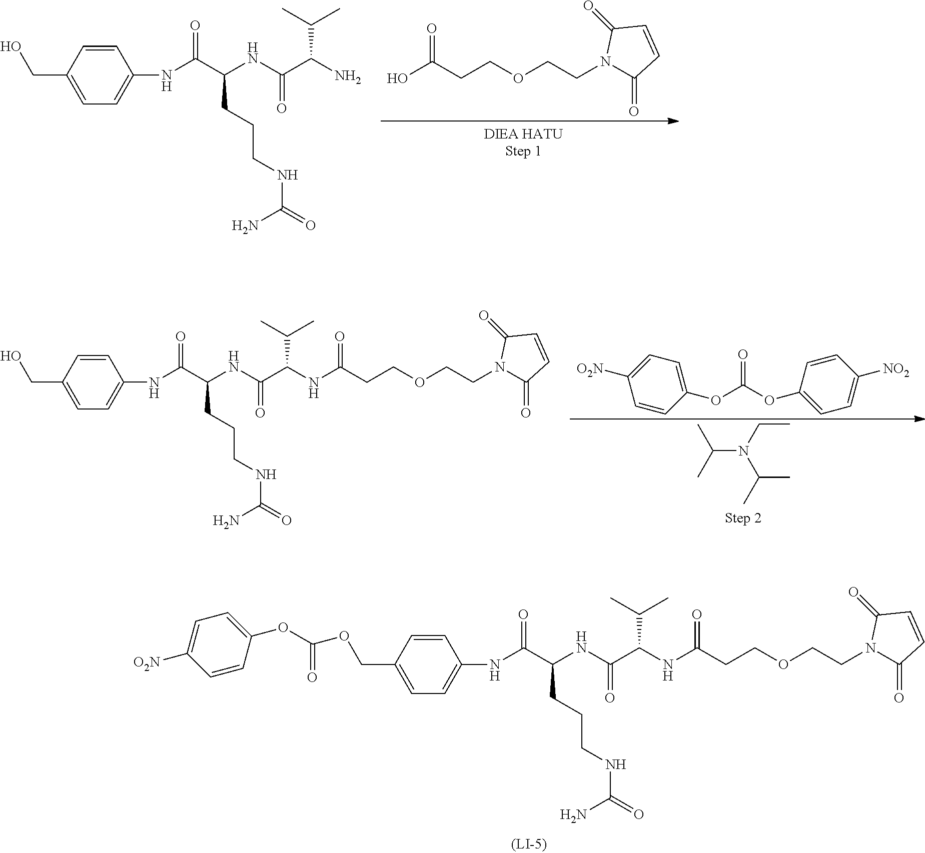

Step 1: DIEA (204 mg, 1.6 mmol) was added to a solution of Mal-PEG1-Acid (112 mg, 0.53 mmol) in DMF (10 ml), followed by the addition of 1-[Bis(dimethylamino)methylene]-1H-1,2,3-triazolo[4,5-b]pyridinium 3-oxide hexafluorophosphate (HATU) (200 mg, 0.53 mmol). The mixture was stirred at room temperature for 5 mins and then was added to a solution of (S)-2-((S)-2-amino-3-methylbutanamido)-N-(4-(hydroxymethyl)phenyl)-5-ureidopentanamide (valcit-pab-OH) (purchased from Levena Biopharma, San Diego) (200 mg, 0.527 mmol) in DMF (5 ml). The mixture was stirred at room temperature for 1 h and then concentrated and purified by reverse phase ISCO using 50 g C18 column, eluted with 10-40% acetonitrile-H2O containing 0.05% TFA. Fractions containing the desired product were concentrated to obtain (S)-2-((S)-2-(3-(2-(2,5-dioxo-2,5-dihydro-1H-pyrrol-1-yl)ethoxy)propanamido)-3-methylbutanamido)-N-(4-(hydroxymethyl)phenyl)-5-ureidopentanamide (MPEG1-vc-pab-OH) (190 mg, 57% yield) as a free base. LCMS M+1=575.3, tr=0.658 min.

Step 2: A solution of (S)-2-((S)-2-(3-(2-(2,5-dioxo-2,5-dihydro-1H-pyrrol-1-yl)ethoxy)propanamido)-3-methylbutanamido)-N-(4-(hydroxymethyl)phenyl)-5-ureidopentanamide (MPEG1-valcit-pabOH) (57.5 mg, 0.100 mmol), bis(4-nitrophenyl) carbonate (130 mg, 1.0 mmol) and DIEA (0.056 mL, 0.32 mmol) was stirred at room temperature for 2 days. The mixture was then concentrated and purified by silica gel column, eluted with 2-6% MeOH:DCM and fractions containing the desired product were collected and concentrated to give 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (4-nitrophenyl) carbonate (LI-5) (59 mg, 80% yield). LCMS M+1=740.2, tr=1.02 min.

### Example 1-6: Synthesis of tert-butyl (2S,4S)-2-(((chlorocarbonyl)oxy)methyl)-4-fluoropyrrolidine-1-carboxylate (LI-6)

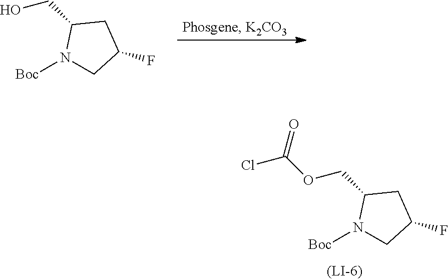 [SMILES: CC(C)(C)OC(=O)N1C[C@@H](F)C[C@H]1CO ; CC(C)(C)OC(=O)N1C[C@@H](F)C[C@H]1COC(=O)Cl]

To a dry flask was introduced potassium carbonate (257 mg, 1.7 equiv), followed by toluene (5 mL). Phosgene in toluene (2.4 mL, 15% in toluene, 3.0 equiv) was added under nitrogen at −35° C. To this vigorously stirred suspension was added dropwise a solution of (2S,4S)-tert-butyl 4-fluoro-2-(hydroxymethyl)pyrrolidine-1-carboxylate (1.093 mmol, 1.0 equiv) in toluene (3.6 ml). Upon completion of the addition, the mixture was stirred at low temperature (˜−35° C. to 0° C.) for 30 mins. The cool bath was removed, and the mixture stirred for a further 1h at room temperature and then filtered by syringe filters with 0.45 micron pore. The volatiles were removed under vacuum with rotary evaporator and the resultant clear pare yellow oil was used directly without further purification.

### Example 1-7: Synthesis of Ketone-Coenzyme A Analog (LI-7)

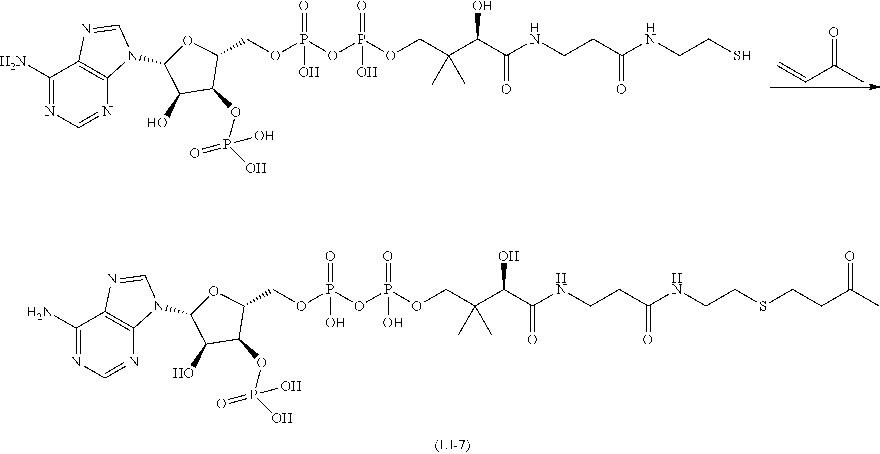

Coenzyme A trilithium salt (259 mg, Sigma, assay>93%) was dissolved in 2.0 mL of 100 mM phosphate buffer (pH 7.5) containing 5 mM EDTA, followed by addition of 3-buten-2-one (29.0 μL, Aldrich, 99%). The reaction was carried out for 75 min at 20° C. Next, the reaction mixture was loaded onto a reverse phase RediSep Rf Gold® C18Aq column (Teledyne Isco), where the product eluted at 100% H₂O. Product-containing fractions were combined and lyophilized, affording linker intermediate (LI-7) as crystalline solid. MS (ESI+) m/z 838.2 (M+1). H-NMR (400 MHz, D₂O) δ 8.525 (s, 1H), 8.235 (s, 1H), 6.140 (d, 1H, J=7.2 Hz), 4.746 (m, 1H), 4.546 (bs, 1H), 4.195 (bs, 1H), 3.979 (s, 1H), 3.786 (dd, 1H, J=4.8, 9.6 Hz), 3.510 (dd, 1H, J=4.8, 9.6 Hz), 3.429 (t, 2H, J=6.6 Hz), 3.294S (t, 2H, J=6.6 Hz), 2.812 (t, 2H, J=6.8 Hz), 2.676 (t, 2H, J=6.8 Hz), 2.604 (t, 2H, J=6.8 Hz), 2.420 (t, 2H, J=6.6 Hz), 2.168 (s, 3H), 0.842 (s, 3H), 0.711 (s, 3H) (note: some peaks which overlap with D₂O are not reported).

### Example 1-8: Synthesis of 4-((tert-butoxycarbonyl)amino)butanoic anhydride (LI-8)

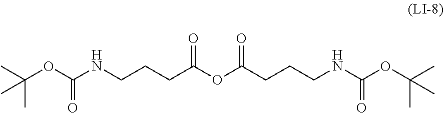 [SMILES: CC(C)(C)OC(=O)NCCCC(=O)OC(=O)CCCNC(=O)OC(C)(C)C]

A solution of DCC (0.53 g, 2.56 mmol) in anhydrous dichloromethane (5 ml) was added via syringe to a solution of 4-((tert-butoxycarbonyl)amino)butanoic acid (1.0 g, 4.9 mmol) in anhydrous dichloromethane (30 ml). After 1 hr of stirring, precipitation of urea was filtered through a syringe filter and the solvent was removed under vacuum. 4-((tert-butoxycarbonyl)amino)butanoic anhydride (LI-8) (1 g, 105% yield) was obtained as a white solid and used without further purification.

### Example 1-9: Synthesis of (((4-((S)-2-((S)-2-(3-(2,5-dioxo-2,5-dihydro-1H-pyrrol-1-yl)propanamido)-3-methylbutanamido)-5-ureidopentanamido)benzyl)oxy)carbonyl)glycine (LI-9)

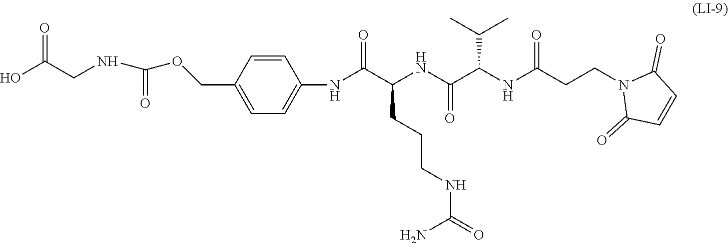 [SMILES: CC(C)[C@H](NC(=O)CCN1C(=O)C=CC1=O)C(=O)N[C@@H](CCCNC(N)=O)C(=O)Nc1ccc(COC(=O)NCC(=O)O)cc1]

DIEA (25.8 mg, 0.2 mmol) was added to glycine (16.7 mg, 0.06 mmol) dissolved in 1 mL DMF and Linker intermediate (LI-3) (34.8 mg, 0.05 mmol) was added, followed by HOAT (8.2 mg, 0.06 mmol). The mixture was then stirred at rt overnight. After completion DMF was removed under reduced pressure, and the crude product was purified by reverse phase ISCO, eluted with 5-50% acetonitrile-H₂O. Fractions containing the desired product were combined and lyophilized to obtain (((4-((S)-2-((S)-2-(3-(2,5-dioxo-2,5-dihydro-1H-pyrrol-1-yl)propanamido)-3-methylbutanamido)-5-ureidopentanamido)benzyl)oxy)carbonyl)glycine (LI-9) (16.4 mg, 49% yield). LCMS M+1=632.3, tr=0.714 min.

### Example 2: Synthesis of Cyclic Dinucleotide (CDN) Intermediates

### Example 2-1: Synthesis of 2-(methylamino)ethyl (9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamate (CDNI-1)

Step 1:

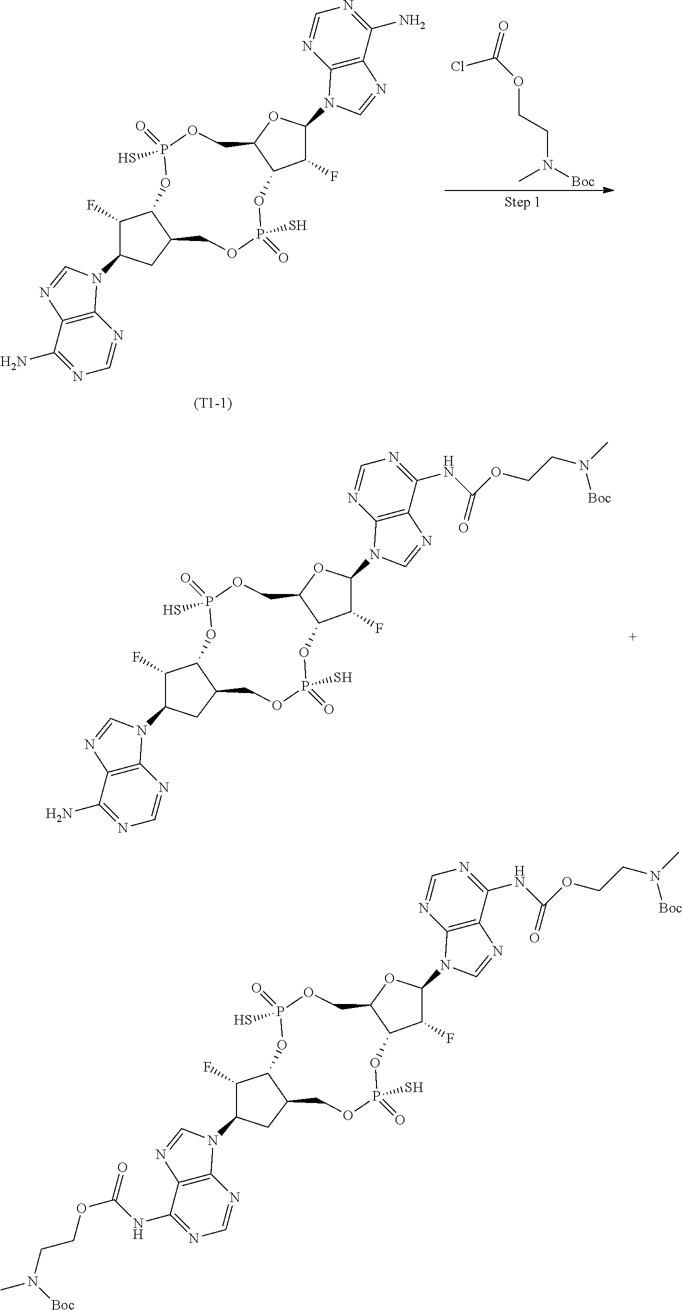

To a solution of phosgene 15% in toluene (14.4 ml, 21.7 mmol) in anhydrous DCM (30 ml) at −78° C. was added a solution of tert-butyl (2-hydroxyethyl)(methyl)carbamate (1.76 g, 10.0 mmol) and pyridine (1.85 ml, 23.4 mmol) in DCM (10 ml). The mixture was stirred at −78° C. for 10 min, warmed to room temperature, stirred for an additional 20 mins and then concentrated and residual solvent was further removed under vacuum. Compound (T1-1) Et₃N salt (300 mg, 0.334 mmol) was dissolved in pyridine (5 ml) and then added to the residue and the mixture was stirred at room temperature for 1 hour resulting in approximately 60% conversion with ˜30% diadduct. Water was added to the mixture, and the mixture was stirred for 10 mins and then concentrated. The residue was suspended in DMSO and purified by ISCO using 15.5 g C18 aq column, eluted with ACN-water 5-50%, aq phase containing 10 mM HOAc-Et₃N. Fractions containing the monoadduct Et₃N salt and were collected and concentrated. (131 mg) LCMS M+1=896.1, tr=0.770 min. ¹H NMR (500 MHz, Methanol-d₄) δ 8.96 (d, J=6.0 Hz, 1H), 8.64 (s, 1H), 8.57 (s, 1H), 8.42 (s, 1H), 8.18 (s, 1H), 6.44 (d, J=16.8 Hz, 1H), 6.36 (d, J=17.3 Hz, 1H), 5.46 (ddd, J=51.9, 15.5, 3.8 Hz, 2H), 5.24-4.99 (m, 2H), 4.64-4.50 (m, 2H), 4.47-4.30 (m, 4H), 4.00 (dt, J=10.3, 4.8 Hz, 2H), 3.64 (t, J=5.9 Hz, 2H), 3.58 (s, 2H), 3.18 (q, J=7.3 Hz, 22H), 3.01-2.83 (m, 7H), 1.46 (s, 8H), 1.41 (d, J=7.6 Hz, 10H), 1.29 (t, J=7.3 Hz, 35H).
Note: Fractions containing the diadduct were collected and concentrated (218 mg). LCMS M+1=1097.1, tr=0.958 min). Monoadduct and starting compound (T1-1) were then obtained by treating the diadduct with NaOH. Specifically the diadduct was dissolved in ACN (10 ml) and then water (20 ml) was added, followed by 1.2 g NaOH. The mixture was stirred at 50° C. for 4h hours, neutralized with 10% HCl and then concentrated. The residue was purified by reverse phase ISCO C18 column, eluted with 10-40 acetonitrile-H₂O containing 10 mM Et₃N HOAc to give monoadduct (106 mg).

Step 2:

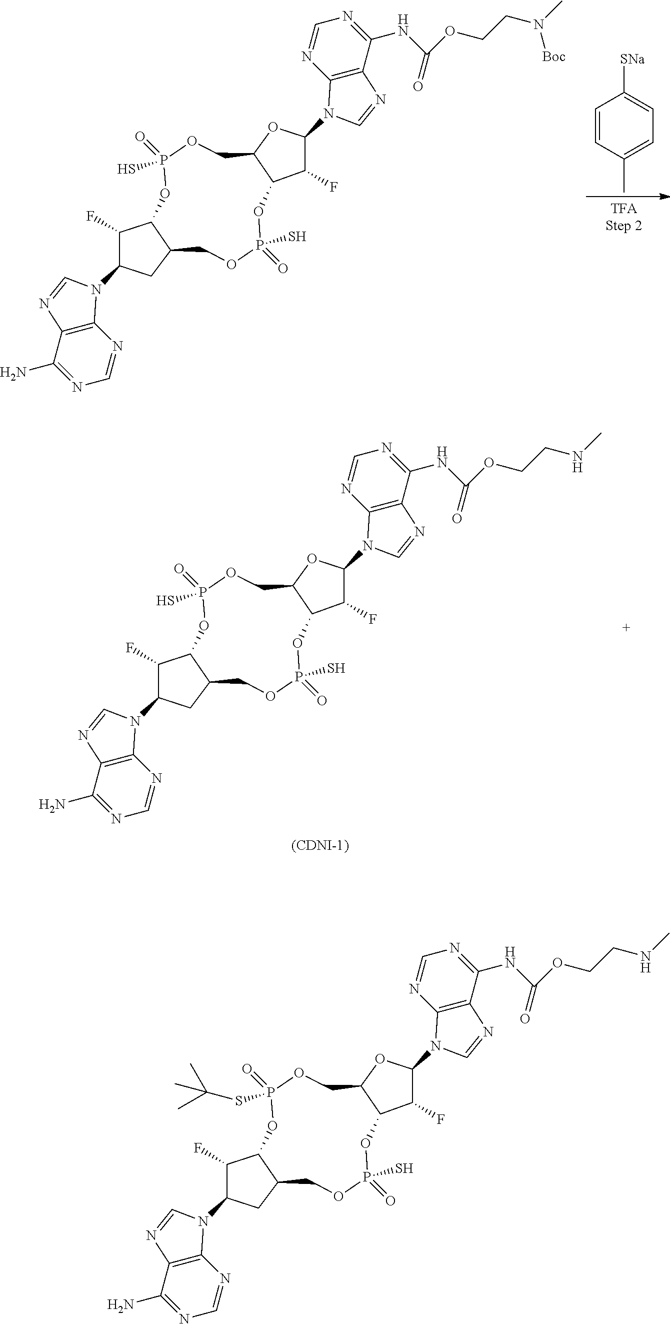

To a flask containing 4-methylbenzenethiol sodium salt (318 mg, 2.16 mmol) was added TFA (5 ml) and the mixture was stirred until near complete dissolution of the solid. This mixture was then added to a flask containing the monoadduct from Step 1 (237 mg, 0.216 mmol) and the mixture was stirred for 2 mins and then concentrated. LCMS showed full Boc deprotection, however approximately ⅓ of t-butylthio adduct remained. The residue was dissolved in DMSO and purified by ISCO using C18 aq column, eluted with 5-30% ACN-water containing 0.05% TFA. Fractions containing the desired product were collected and concentrated to give (CDNI-1) (107 mg, 39.2% yield) (LCMS M+1=796.1, tr=0.555 min). 1H NMR (500 MHz, DMSO-d6) b 10.34 (s, 1H), 8.83 (b, 7H), 8.09 (s, 1H), 6.41 (d, J=15.2 Hz, 1H), 6.30 (d, J=15.2 Hz, 1H), 5.70-5.51 (m, 1H), 5.44 (d, J=51.8 Hz, 1H), 5.03 (d, J=25.7 Hz, 2H), 4.49-4.33 (m, 4H), 4.27 (s, 2H), 3.90-3.55 (m, 2H), 3.10 (d, J=51.8 Hz, 1H), 2.91-2.57 (m, 2H)
Note: Fractions containing the t-butylthio adduct (LCMS M+1=852.1, tr=0.792 min) were collected and after standing for 3 days the t-butylthio adduct converted to (CDNI-1) (37 mg, 0.029 mmol, 13% yield).

### Example 2-1: Synthesis of 4-((S)-2-((S)-2-amino-3-methylbutanamido)-5-ureidopentanamido)benzyl (2-(((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamoyl)oxy)ethyl)(methyl)carbamate (CDNI-2)

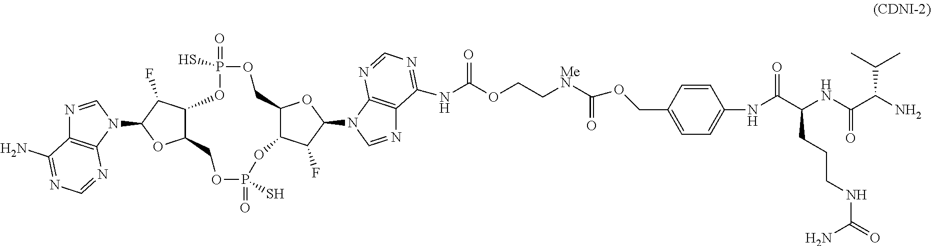 [SMILES: CC(C)[C@H](N)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Step 1: 4-((S)-2-((S)-2-((((9H-fluoren-9-yl)methoxy)carbonyl)amino)-3-methylbutanamido)-5-ureidopentanamido)benzyl 2-(4-nitrophenyl)acetate (Fmoc-Val-Cit-PABC-PNP) (23.18 mg, 0.030 mmol) (purchased from Levena Biopharma, San Diego), DIEA (0.024 mL, 0.137 mmol) and 3-Hydroxytriazolo[4,5-b]pyridine (HOAT) (3.74 mg, 0.027 mmol) were added to a round bottom flask containing (CDNI-1) (25 mg, 0.027 mmol) in DMF (2 mL). The reaction was stirred at room temperature for 4 hours and then heated to 45° C. and stirred for an additional hour. The mixture was then concentrated and the residue purified by ISCO using 15.5 g C18 aq column, eluted with 5-60% ACN-water with 0.05% TFA. Fmoc-vc-pabc-(CDNI-2) (34.4 mg, 81% yield) was obtained. LCMS M/2+1=712.3, tr=1.007 min.

Step 2: Piperidine (0.200 ml) was added to a solution of Fmoc-vc-pabc-(CDNI-2) (34.4 mg, 0.022 mmol) TFA salt in DMF (5 mL) and the mixture was stirred at room temperature for 30 mins, and then concentrated. The residue was purified by reverse phase ISCO using C18 aq column, eluted with 5-35% acetonitrile-H₂O containing 0.05% TFA. Fractions containing desired product were concentrated to (CDNI-2) (31.1 mg, 92% yield) as TFA salt. LCMS M+1=1201.2 tr=0.671 min.

### Example 2-3: Synthesis of (CDNI-3)

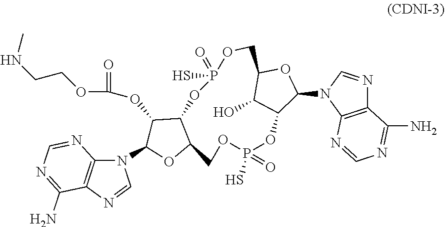 [SMILES: CNCCOC(=O)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(N)ncnc21)[C@@H]3O]

Step 1: a) Et₃N (1 ml) was added to Compound (T1-2) ammonium salt (400 mg, 0.552 mmol) in pyridine (30 ml) and the mixture was concentrated. The procedure was repeated twice to obtain the triethylammonium salt of Compound (T1-2).

b) A solution of tert-butyl (2-hydroxyethyl)(methyl)carbamate (290 mg, 1.66 mmol) in DCM (10 ml) with pyridine (0.313 mL, 3.86 mmol) was added to a solution of 15% phosgene solution in toluene (4.4 ml) in DCM (20 ml) at −78° C. and the mixture was stirred for 15 mins and then warmed to room temperature and concentrated to obtain 2-((tert-butoxycarbonyl) (methyl)amino)ethyl carbonochloridate.

Step 2: Compound (T1-2) Et₃N salt was resuspended in anhydrous pyridine (30 ml) and then added to 2-((tert-butoxycarbonyl)(methyl)amino)ethyl carbonochloridate from step 1 b) and the mixture was stirred at room temperature for 30 mins. Water was then added and the mixture was concentrated. The residue was suspended in DMSO-water and then purified by reverse phase ISCO using C18 column, 15.5 g aq column, eluted with 2-40% acetonitrile-H₂O containing 10 mM Et₃N HOAc. The fractions containing the desired Boc protected monoadduct (387 mg, 57.7% yield) were collected and lyophilized. M+1=892.2. tr=0.770 min. 1H NMR (500 MHz, Methanol-d₄) δ 8.83 (s, 1H), 8.34 (s, 1H), 8.24 (s, 1H), 8.18 (s, 1H), 6.33 (dd, J=25.9, 6.9 Hz, 2H), 6.10 (s, 1H), 5.51 (s, 1H), 5.33 (s, 1H), 4.68 (s, 1H), 4.51-4.14 (m, 7H), 4.03 (d, J=9.5 Hz, 1H), 3.70-3.56 (m, 1H), 3.45 (s, 2H), 3.17 (d, J=7.3 Hz, 22H), 2.88 (s, 4H), 1.40 (s, 4H), 1.29 (t, J=7.3 Hz, 33H).

Step 3: TFA (5 mL) was added to a flask containing 4-methylbenzenethiol sodium salt (200 mg, 1.36 mmol) and the mixture was stirred until complete dissolution. The mixture was then added to another flask containing the Boc protected mono-adduct from step 2 (250 mg, 0.228 mmol) and after 1 min at room temperature the TFA was removed. The mixture was then dissolved in DMSO and purified by reverse phase ISCO using 15 g C18 aq column, eluted with 2-20% acetonitrile-H₂O containing 0.05% TFA. The fractions containing desired product were concentrated to obtain the de-protected monoadduct (CDNI-3) as a TFA salt. LCMS M+1=792.0, tr=0.611 min. ¹H NMR (500 MHz, DMSO-d₆) δ 9.37 (d, J=41.6 Hz, 2H), 8.89 (s, 1H), 8.70 (s, 1H), 8.43 (s, 1H), 8.30 (s, 1H), 6.33 (d, J=7.8 Hz, 1H), 6.21 (d, J=8.2 Hz, 1H), 5.51-5.24 (m, 2H), 4.72-4.62 (m, 1H), 4.49 (s, 1H), 4.41 (s, 1H), 4.31 (s, 3H), 4.07 (s, 2H), 3.85 (s, 1H), 3.43 (s, 1H), 3.23 (s, 1H), 2.67 (s, 2H).
Note: Fractions containing the t-butylthio adduct were collected and after standing for 3 days the t-butylthio adduct converted to (CDNI-3)

### Example 2-4: Synthesis of (CDNI-4)

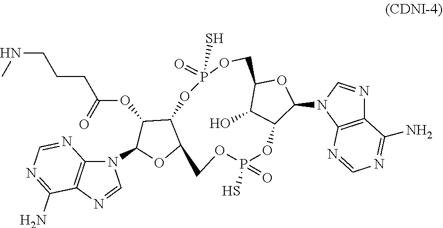 [SMILES: CNCCCC(=O)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(N)ncnc21)[C@@H]3O]

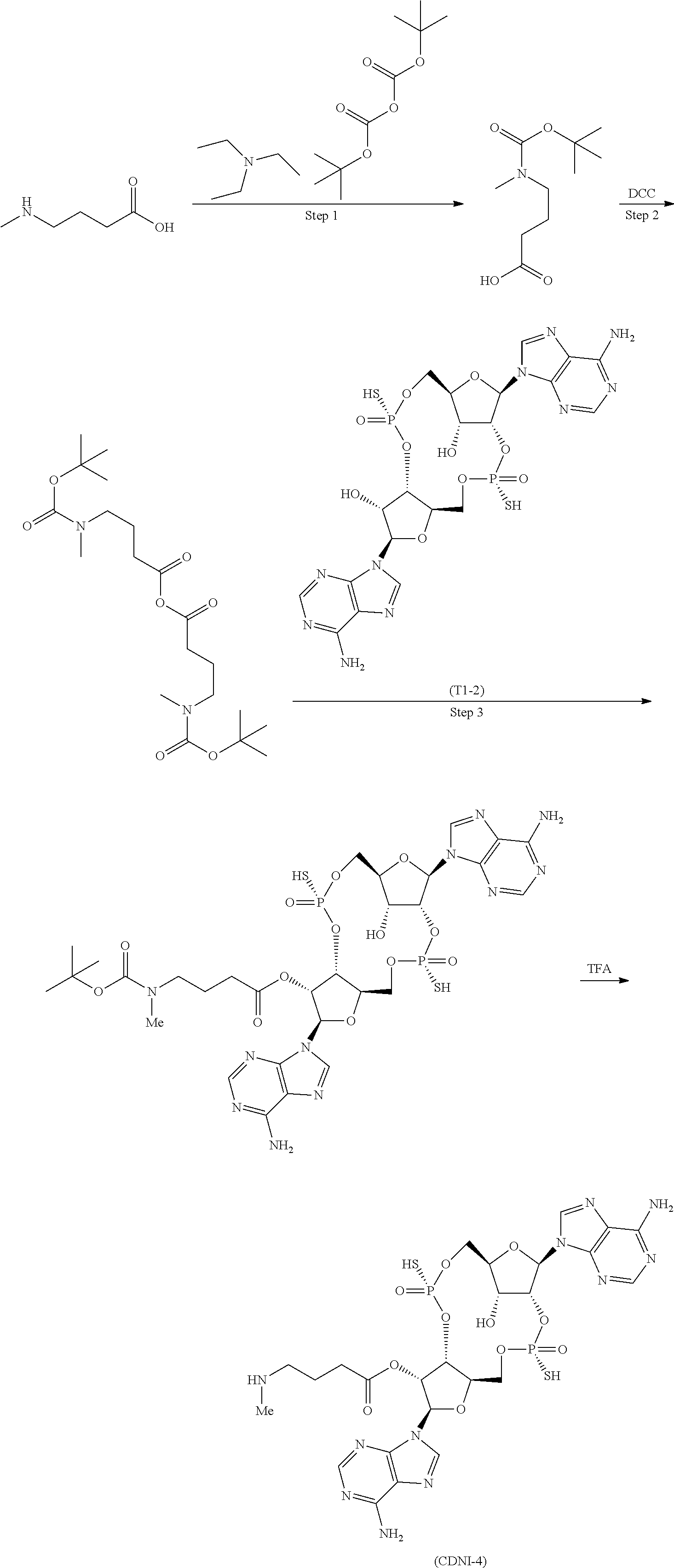

Step 1: Di-t-butyl dicarbonate (4.26 g, 19.5 mmol) was added dropwise over 10 minutes to a mixture of 4-(methylamino)butyric acid hydrochloride (2.0 g, 13.0 mmol) in MeOH (25 mL) and Et₃N (7.26 mL, 52.1 mmol). The reaction mixture was stirred at room temperature for 22 hrs and then concentrated. The residue was dissolved in EtOAc (100 mL), and washed with an ice-cold 0.1 N HCl solution (20.0 mL). The organic layer was then washed with water to neutral pH, and then washed with sat. NaCl. The EtOAc layer was dried over Na₂SO₄ and concentrated to give 4-((tert-butoxycarbonyl)(methyl)amino)butanoic acid (2.08 g, 70%). 1H NMR (500 MHz, Chloroform-d) δ 3.28 (t, J=6.9 Hz, 2H), 2.84 (s, 3H), 2.35 (t, J=7.2 Hz, 2H), 1.84 (p, J=7.1 Hz, 2H), 1.45 (s, 9H).

Step 2: A solution of dicyclohexylcarbodiimide (704 mg, 3.41 mmol) in 10 ml of anhydrous DCM was added drop wise under N₂ to a flask containing 4-((tert-butoxycarbonyl)(methyl)amino)butanoic acid (1.43 g, 6.56 mmol) in anhydrous DCM (20 ml). The mixture was stirred for 2 hrs and then concentrated to about 15 mL, filtered and the solvent removed under vacuum. The crude was filtered through 0.45 micron filter twice to yield 4-((tert-butoxycarbonyl)(methyl)amino)butanoic anhydride as a clear pale yellow oil (1.36 g, 99% yield). ¹H NMR (500 MHz, Chloroform-d) δ 3.28 (t, J=6.9 Hz, 2H), 2.84 (s, 3H), 2.46 (t, J=7.3 Hz, 2H), 1.87 (p, J=7.2 Hz, 2H), 1.45 (s, 9H).

Step 3: 4-((tert-butoxycarbonyl)(methyl)amino)butanoic anhydride (152.0 mg, 0.366 mmol) in DMF (1.6 mL) was added to Compound (T1-2) (63.1 mg, 0.091 mmol) in pyridine (0.8 mL). The reaction mixture was stirred at room temperature for 3 days and then the solvent was removed. The residue was purified by reverse phase ISCO using C18 column, 50 g aq column, eluted with 5-50% MeCN/water (containing 10 mM Et₃N HOAc). Fractions containing desired boc protected monoadduct were collected and lyophilized (45.3 mg, 56% yield). LCMS M+1=890.20, tr=0.787 min.

Step 4: TFA (2 mL) was added to a flask containing 4-methylbenzenethiol sodium salt and the mixture was stirred until complete dissolution and then added to another flask containing the boc protected monoadduct from step 3. TFA was immediately removed and the mixture was then dissolved in DMSO and purified by reverse phase ISCO C18 column, 15 g C18 aq column, eluted with 2-20% acetonitrile-H₂O containing 0.05% TFA. Fractions containing desired product were concentrated to obtain (CDNI-4) (35.0 mg, 89% yield) as TFA salt. LCMS M+1=790.2, tr=0.220 min.

### Example 2-5: Synthesis of (CDNI-5)

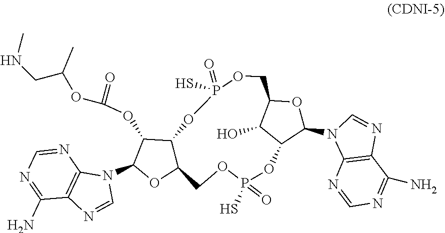 [SMILES: CNCC(C)OC(=O)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(N)ncnc21)[C@@H]3O]

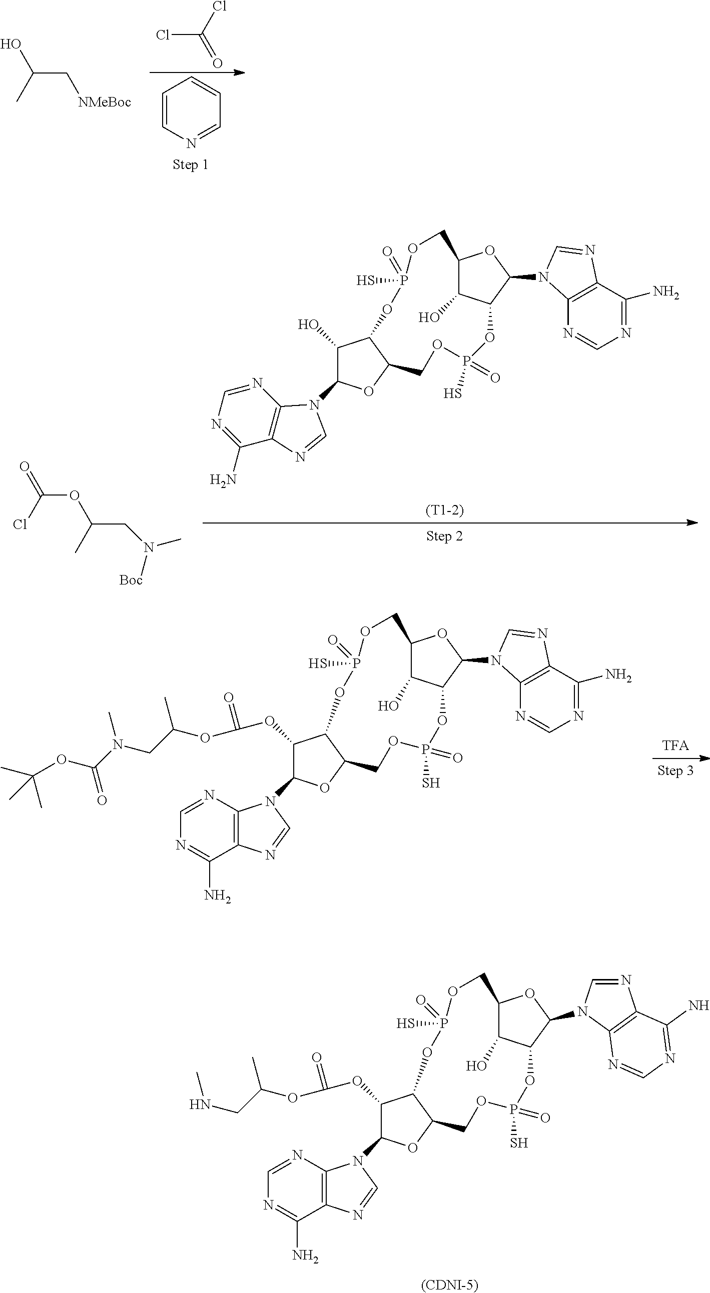

Step 1: a) Compound (T1-2) (20 mg, 0.028 mmol) ammonium salt was dissolved in 5 ml pyridine and 0.06 ml Et₃N was then added. The mixture was concentrated and the process repeated twice to obtain the Compound (T1-2) triethylammonium salt.

b) A solution of tert-butyl (2-hydroxyethyl)(methyl)carbamate (84 mg, 0.44 mmol) in DCM (3 ml) with pyridine (0.072 mL, 0.88 mmol) was added to a solution of 15% phosgene solution in toluene (0.88 ml) in DCM (10 ml) at −78° C. The mixture was stirred for 15 mins, then warmed to room temperature and concentrated to give 1-((tert-butoxycarbonyl) (methyl)amino)propan-2-yl carbonochloridate.

Step 2: Compound (T1-2) Et₃N salt was resuspended in anhydrous pyridine (1 ml) and then added to 1-((tert-butoxycarbonyl)(methyl)amino)propan-2-yl carbonochloridate. The mixture was stirred for 30 mins and then water was added. The mixture was concentrated, dissolved in DMSO-water and purified by reverse phase ISCO using C18 column, 15.5 g aq column, eluted with 2-40% acetonitrile-H₂O containing 10 mM Et₃N HOAc. Fractions containing desired Boc protected monoadduct were collected and lyophilized (33 mg, 43% yield). M+1=906.1, tr=0.785 min.

Step 3: TFA (2 mL) was added to a flask containing 4-methylbenzenethiol sodium salt and the mixture was stirred until complete dissolution and then added to another flask containing the boc protected monoadduct from step 3 (33 mg, 0.030 mmol. TFA was immediately removed and the mixture was then dissolved in DMSO and purified by reverse phase ISCO using 15.5 g C18 aq column, eluted with 2-20% acetonitrile-H₂O containing 0.05% TFA. Fractions containing desired product were concentrated to obtain (CDNI-5) (21 mg, 55% yield) as TFA salt. LCMS M+1=806.0, tr=0.586 min.

### Example 2-6: Synthesis of (CDNI-6)

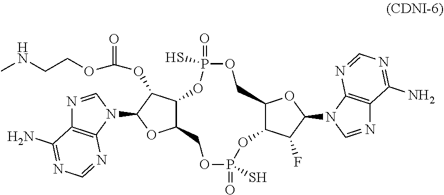 [SMILES: CNCCOC(=O)O[C@@H]1[C@@H]2O[P@](=O)(S)OC[C@H]3O[C@@H](n4cnc5c(N)ncnc54)[C@H](F)[C@@H]3O[P@](=O)(S)OC[C@H]2O[C@H]1n1cnc2c(N)ncnc21]

Intermediate (CDNI-6) was prepared using the methods described for the synthesis of intermediate (CDNI-3), except Compound (T1-5) was used in place of Compound (T1-2). Intermediate (CDNI-6) (25.6 mg, 66.8% yield) as TFA salt. LCMS M+1=794.1, tr=0.518 min.

### Example 2-7: Synthesis of (CDNI-7)

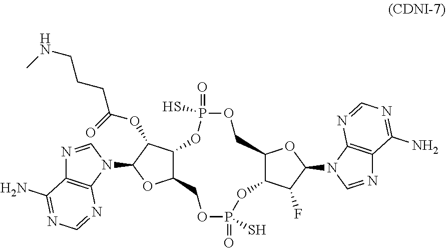 [SMILES: CNCCCC(=O)O[C@@H]1[C@@H]2O[P@](=O)(S)OC[C@H]3O[C@@H](n4cnc5c(N)ncnc54)[C@H](F)[C@@H]3O[P@](=O)(S)OC[C@H]2O[C@H]1n1cnc2c(N)ncnc21]

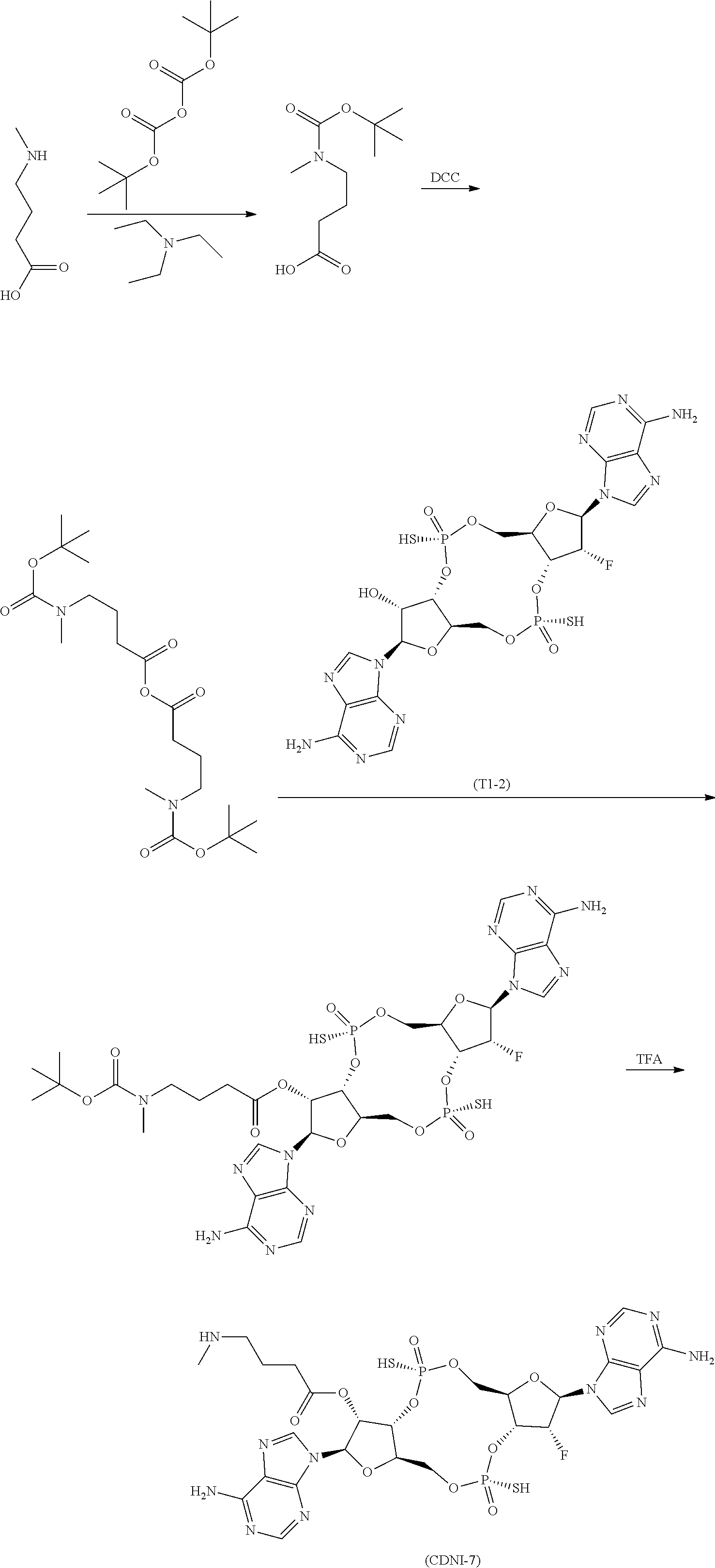

Intermediate (CDNI-7) was prepared using the methods described for the synthesis of intermediate (CDNI-4), except Compound (T1-5) was used in place of Compound (T1-2). Intermediate (CDNI-7) (10.0 mg, 8% yield) as TFA salt. LCMS M+1=792.2, tr=0.381 min.

### Example 2-8: Synthesis of (CDNI-8)

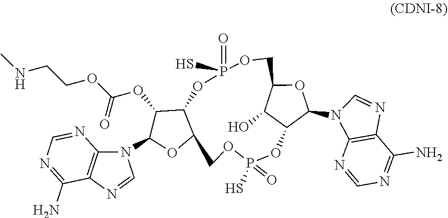 [SMILES: CNCCOC(=O)O[C@@H]1[C@@H]2O[P@@](=O)(S)OC[C@H]3O[C@@H](n4cnc5c(N)ncnc54)[C@H](O[P@](=O)(S)OC[C@H]2O[C@H]1n1cnc2c(N)ncnc21)[C@@H]3O]

Intermediate (CDNI-8) was prepared using the methods described for the synthesis of intermediate (CDNI-3), except Compound (T1-3) was used in place of Compound (T1-2).

### Example 2-9: Synthesis of (CDNI-9a) and of (CDNI-9b)

### a) Synthesis of (CDNI-9a):

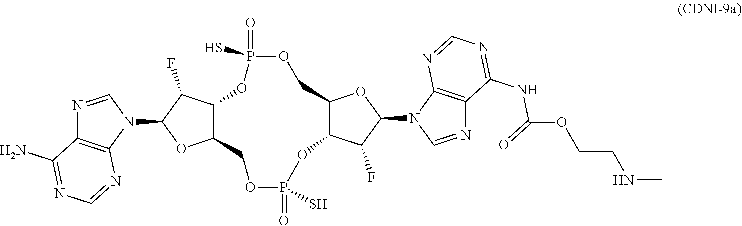 [SMILES: CNCCOC(=O)Nc1ncnc2c1ncn2[C@@H]1O[C@@H]2CO[P@](=O)(S)O[C@H]3[C@@H](F)[C@H](n4cnc5c(N)ncnc54)O[C@@H]3CO[P@@](=O)(S)O[C@H]2[C@H]1F]

Intermediate (CDNI-9a) was prepared using the methods described for the synthesis of intermediate (CDNI-3), except Compound (T1-6) was used in place of Compound (T1-2). Intermediate (CDNI-9a) (32.1 mg, 39.0% yield) (LCMS M+1=796.0, tr=0.406 min). However, Step 1 for the preparation of 2-((tert-butoxycarbonyl)(methyl)amino)ethyl carbonochloridate was modified as follows:

Tert-butyl (2-hydroxyethyl)(methyl)carbamate (175 mg, 0.736 mmol) and K2CO3 (43 mg, 0.626 mmol) were added to a flask under N₂., and then toluene (10 mL) was added and the mixture cooled to −15° C. The mixture was stirred and a solution of phosgene in toluene (1.1 mmol, 15% in toluene) was added dropwise. The mixture was stirred at low temperature (−15° C. to 0° C.) for an additional 30 mins, warmer to room temperature and stirred for another hour. The mixture was filtered by syringe filter (0.45 micron pore), and the solvent was removed to give 2-((tert-butoxycarbonyl)(methyl)amino)ethyl carbonochloridate as a clear pare yellow oil which was used directly without further purification.

### b) Synthesis of (CDNI-9b):

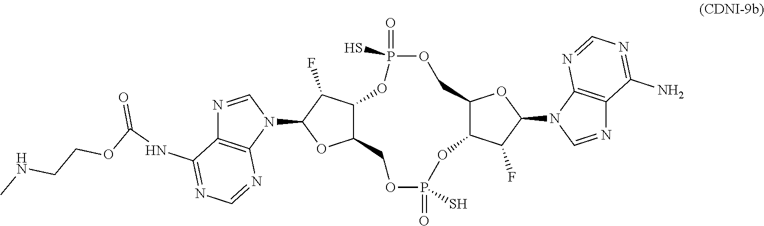 [SMILES: CNCCOC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@](=O)(S)O[C@H]2[C@H]1F]

Intermediate (CDNI-9b) was also obtained during the synthesis of Intermediate (CDNI-9a). CDN intermediate (CDNI-9a) and CDN intermediate (CDNI-9b) could not separated. (CDNI-9a). CDN intermediate (CDNI-9a) and CDN intermediate (CDNI-9b) (32.1 mg, 39.0% yield) (LCMS M+1=796.0, tr=0.406 min).

### Example 2-10: Synthesis of (2R,3R,3aR,5R,7aR,9R,10R,10aR,12R,14aR)-2-(6-amino-9H-purin-9-yl)-9-(6-((3-aminopropyl)amino)-9H-purin-9-yl)-3,10-difluoro-5,12-dimercaptooctahydro-2H,7H-difuro[3,2-d:3′,2′-j][1,3,7,9]tetraoxa[2,8]diphosphacyclododecine 5,12-dioxide (CDNI-10)

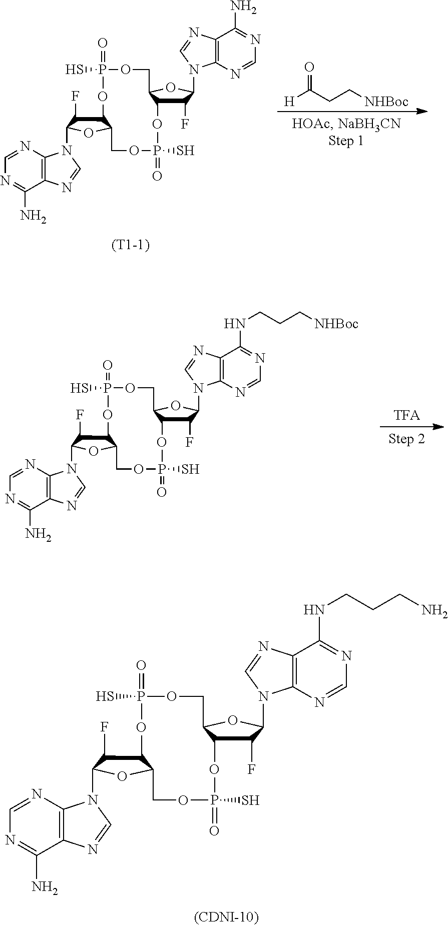

Step 1: HOAc (0.020 ml) and tert-butyl (3-oxopropyl)carbamate (10 mg, 0.058 mmol) were added to a suspension of Compound (T1-1) (5 mg, 0.0056 mmol) in MeOH (1 ml) and the mixture was heated to 50° C. for 16 hours (LCMS showed slow imine formation M+1 850.2 tr=0.680 min) NaBH₃CN (0.35 mg, 0.0056 mmol) was then added and the reaction was stirred at room temperature for 2 hours. LCMS indicated ˜25% conversion. M+1=852.1 tr=0.708 min. An additional 5 mg of tert-butyl (3-oxopropyl)carbamate was added and the mixture was heated at 50° C. for 2 hours, followed by addition of 5 mg NaBH₃CN. The mixture was stirred for 1 hour and conversion monitored by LCMS. The process of adding 5 mg additional tert-butyl (3-oxopropyl)carbamate and 5 mg additional NaBH₃CN was repeated until ˜50% conversion was achieved. The mixture was concentrated, the residue dissolved in 2 ml MeOH and purified by mass triggered reverse phase HPLC, using C18 column, eluted with 13-29% acetonitrile-H₂O containing 0.05% TFA. Fractions containing desired product were concentrated to obtain tert-butyl (3-((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)amino)propyl)carbamate as TFA salt. LCMS M+1=852.1 tr=0.695 min.

Step 2: tert-butyl (3-((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)amino)propyl)carbamate (1 mg, 0.001 mmol) was treated with TFA (1 ml) and was immediately concentrated. H₂O and ACN (1:1) was added and the sample was lyophilized to give (2R,3R,3aR,5R,7aR,9R,10R,10aR,12R,14aR)-2-(6-amino-9H-purin-9-yl)-9-(6-((3-aminopropyl)amino)-9H-purin-9-yl)-3,10-difluoro-5,12-dimercaptooctahydro-2H,7H-difuro[3,2-d:3′,2′-j][1,3,7,9]tetraoxa[2,8]diphosphacyclododecine 5,12-dioxide (0.9 mg, 30% yield) as TFA salt. LCMS M+1=748.0, tr=0.227 min.

### Example 2-11

### a) Synthesis of ((2S,4S)-4-fluoropyrrolidin-2-yl)methyl (9-((2R,3R,3aR,5R,7aR,9R,10R,10aR,12S,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamate (CDNI-11a)

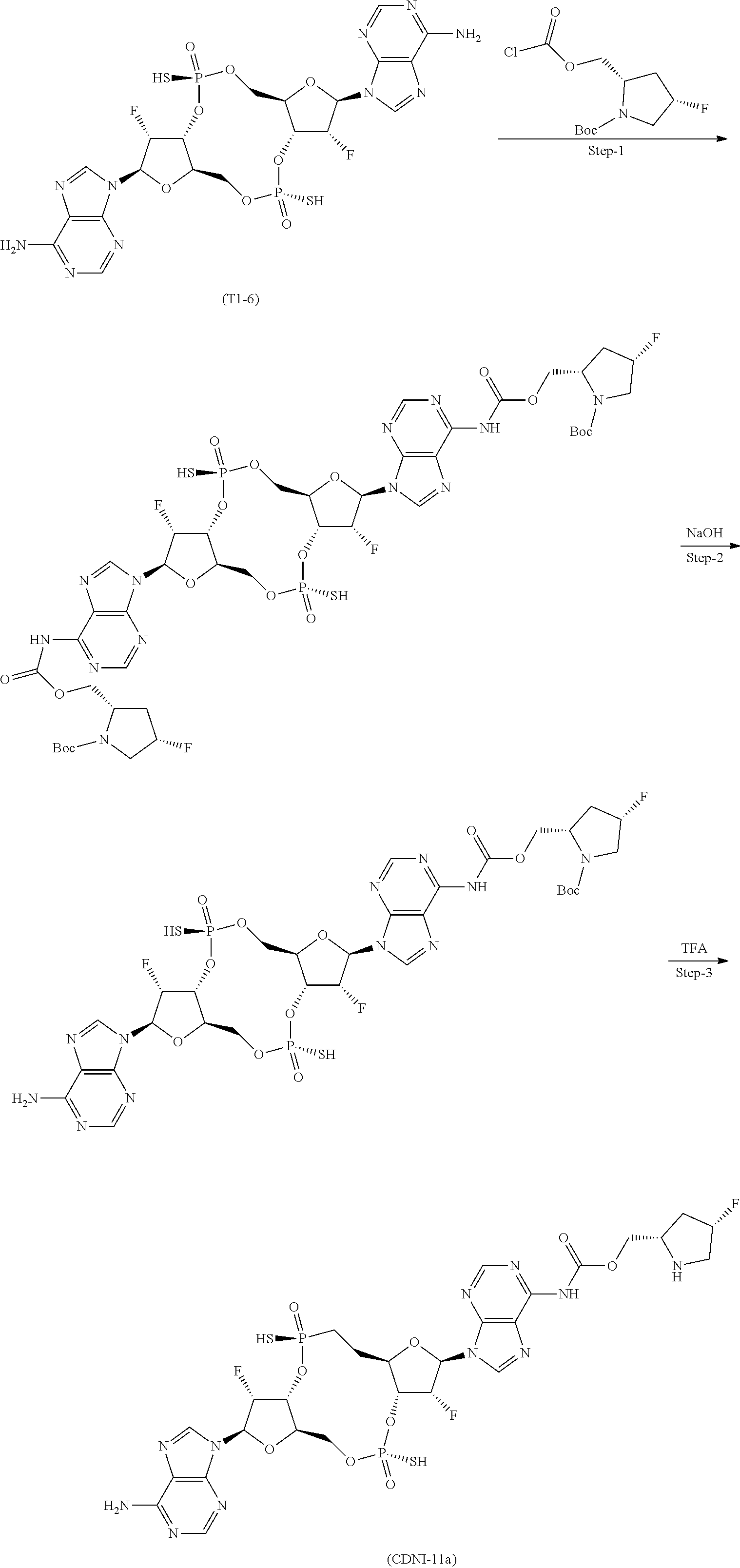

Step 1: Compound (T1-6) Et₃N salt (224 mg, 0.25 mmol) with pyridine (88 uL, 7.0 equiv) in NMP (0.5 mL) and DCM (1.5 mL) was added to (2S,4S)-tert-butyl 2-(((chlorocarbonyl)oxy)methyl)-4-fluoropyrrolidine-1-carboxylate (LI-6) in DCM (1.5 mL) over 5 minutes. The mixture was stirred at room temperature for one hour. Water was added to the reaction and it was stirred for another 10 mins and then concentrated. The mixture was suspended in DMSO and purified by ISCO using 15.5 g C18 aq column, eluted with ACN-water 5-50%, aq phase containing 10 mM HOAc-Et₃N to give the diadduct, di-tert-butyl 5,5′-(((((((2R,3R,3aR,5R,7aR,9R,10R,10aR,12S,14aR)-3,10-difluoro-5,12-dimercapto-5,12-dioxidooctahydro-2H,7H-difuro[3,2-d:3′,2′-j][1,3,7,9]tetraoxa[2,8]diphosphacyclododecine-2,9-diyl)bis(9H-purine-9,6-diyl))bis(azanediyl))bis(carbonyl))bis(oxy))bis(methylene))(3S,3'S,5S,5'S)-bis(3-fluoropyrrolidine-1-carboxylate), (149.5 mg). LCMS M+1=1185.1, tr=0.944 min.

Step 2: The diadduct (149.5 mg) from step 1 was dissolved in ACN (5 ml) and then water (10 ml) was added, followed by 0.6 g NaOH. The mixture was stirred at 50° C. for 4 hours, then neutralized with 4M HCl and then concentrated. The residue was purified by reverse phase ISCO, C18 column, eluted with 10-50 acetonitrile-H₂O containing 10 mM Et₃N HOAc to give the protected monoadduct, tert-butyl (2S,4S)-2-((((9-((2R,3R,3aR,5R,7aR,9R,10R,10 aR,12S,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamoyl)oxy)methyl)-4-fluoropyrrolidine-1-carboxylate, (32.0 mg). LCMS M+1=940.1, tr=0.750 min.

Step 3: TFA (2.0 ml) was added to a flask containing monoadduct from step 2 (32.0 mg, 0.028 mmol) and the mixture was stirred for 2 mins and then concentrated. The residue was dissolved in DMSO and purified by ISCO using C18 aq column, eluted with 5-30% ACN-water containing 0.05% TFA to give ((2S,4S)-4-fluoropyrrolidin-2-yl)methyl (9-((2R,3R,3aR,5R,7aR,9R,10R,10aR,12S, 14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamate (CDNI-11a) (13.1 mg, 44.0% yield) (LCMS M+1=840.0, tr=0.407 min).

### b) Synthesis of ((2S,4S)-4-fluoropyrrolidin-2-yl)methyl (9-((2R,3R,3aR,5S,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamate (CDNI-11 b)

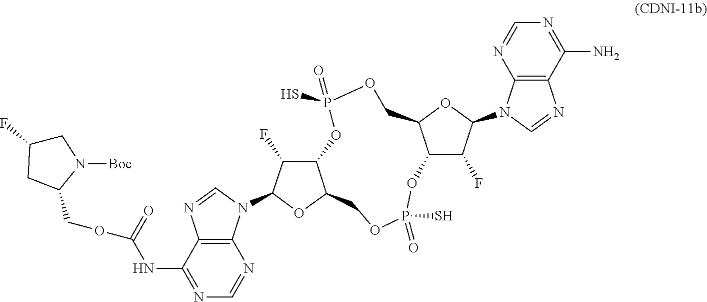 [SMILES: CC(C)(C)OC(=O)N1C[C@@H](F)C[C@H]1COC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@](=O)(S)O[C@H]2[C@H]1F]

Intermediate (CDNI-11b) was also obtained during the synthesis of Intermediate (CDNI-1a). CDN intermediate (CDNI-11a) and CDN intermediate (CDNI-11b) could not separated. (CDNI-1a). CDN intermediate (CDNI-11a) and CDN intermediate (CDNI-9b) (13.1 mg, 44.0% yield) (LCMS M+1=840.0, tr=0.407 min).

### Example 2-12: Synthesis of N-(9-((2R,3R,5R,7aR,9R,10R,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)-4-(methylamino)butanamide (CDNI-12)

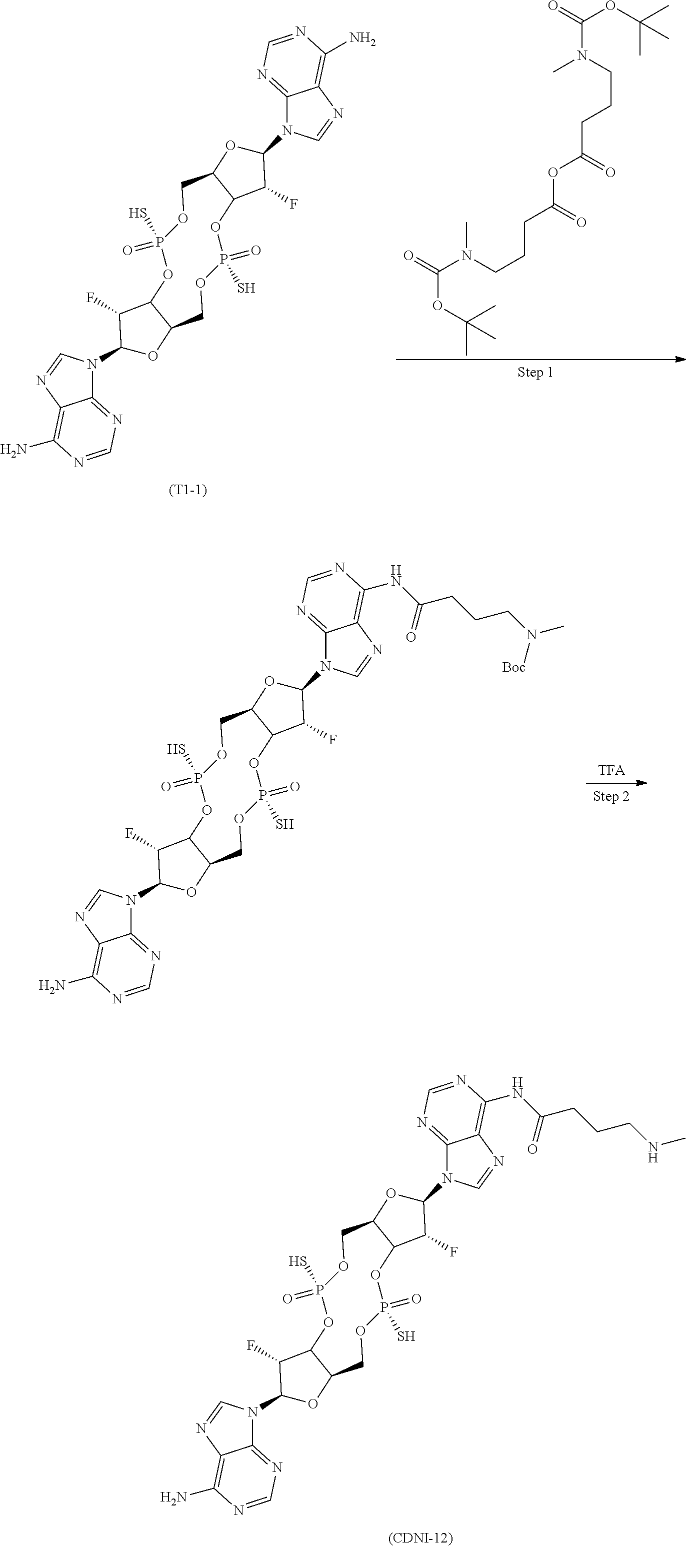 [SMILES: Nc1ncnc2c1ncn2[C@@H]1O[C@@H]2CO[P@@](=O)(S)OC3[C@@H](F)[C@H](n4cnc5c(N)ncnc54)O[C@@H]3CO[P@@](=O)(S)OC2[C@H]1F ; CN(CCCC(=O)OC(=O)CCCN(C)C(=O)OC(C)(C)C)C(=O)OC(C)(C)C ; CN(CCCC(=O)Nc1ncnc2c1ncn2[C@@H]1O[C@@H]2CO[P@@](=O)(S)OC3[C@@H](F)[C@H](n4cnc5c(N)ncnc54)O[C@@H]3CO[P@@](=O)(S)OC2[C@H]1F)C(=O)OC(C)(C)C ; CNCCCC(=O)Nc1ncnc2c1ncn2[C@@H]1O[C@@H]2CO[P@@](=O)(S)OC3[C@@H](F)[C@H](n4cnc5c(N)ncnc54)O[C@@H]3CO[P@@](=O)(S)OC2[C@H]1F ; "(CDNI-12)"]

Step 1: 4-((tert-butoxycarbonyl)(methyl)amino)butanoic anhydride (241 mg, 0.580 mmol) was added to a solution of Compound (T1-1) Et₃N salt (40 mg, 0.045 mmol) in pyridine (5 ml) and heated to 50° C. and stirred for 72 hours. DMAP (10 mg) and 50 mg more anhydride were added and the reaction was stirred at 50° C. for 8 hours and then concentrated and purified using reverse phase ISCO with 15 g C18 aq column, eluted with 5-45% acetonitrile-H₂O (aqueous phase containing 10 mM Et₃N HOAc). Fractions containing desired product were collected and lyophilized to obtain boc-protected intermediate CDNI-12 as an Et₃N salt (8 mg, 16% yield). LCMS M+1=894.0, tr=0.776 min.

Note: 4-((tert-butoxycarbonyl)(methyl)amino)butanoic anhydride was synthesized as described in the synthesis of CDNI-4.

Step 2: TFA (1 ml) was added to a flask containing boc-protected intermediate CDNI-12 Et₃N salt (8 mg, 0.007 mmol) and then immediately concentrated. The residue was purified by reverse phase ISCO using 15 g C18 column, eluted with 5-45% acetonitrile-H₂O containing 0.05% TFA. Fractions containing desired product were concentrated to obtain intermediate CDNI-12 as a TFA salt (3.7 mg, 49.6% yield). LCMS M+1=794.0, tr=0.636 min.

### Example 2-13: Synthesis of 4-amino-N-(9-((2R,3R,5R,7aR,9R,10R,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)butanamide (CDNI-13)

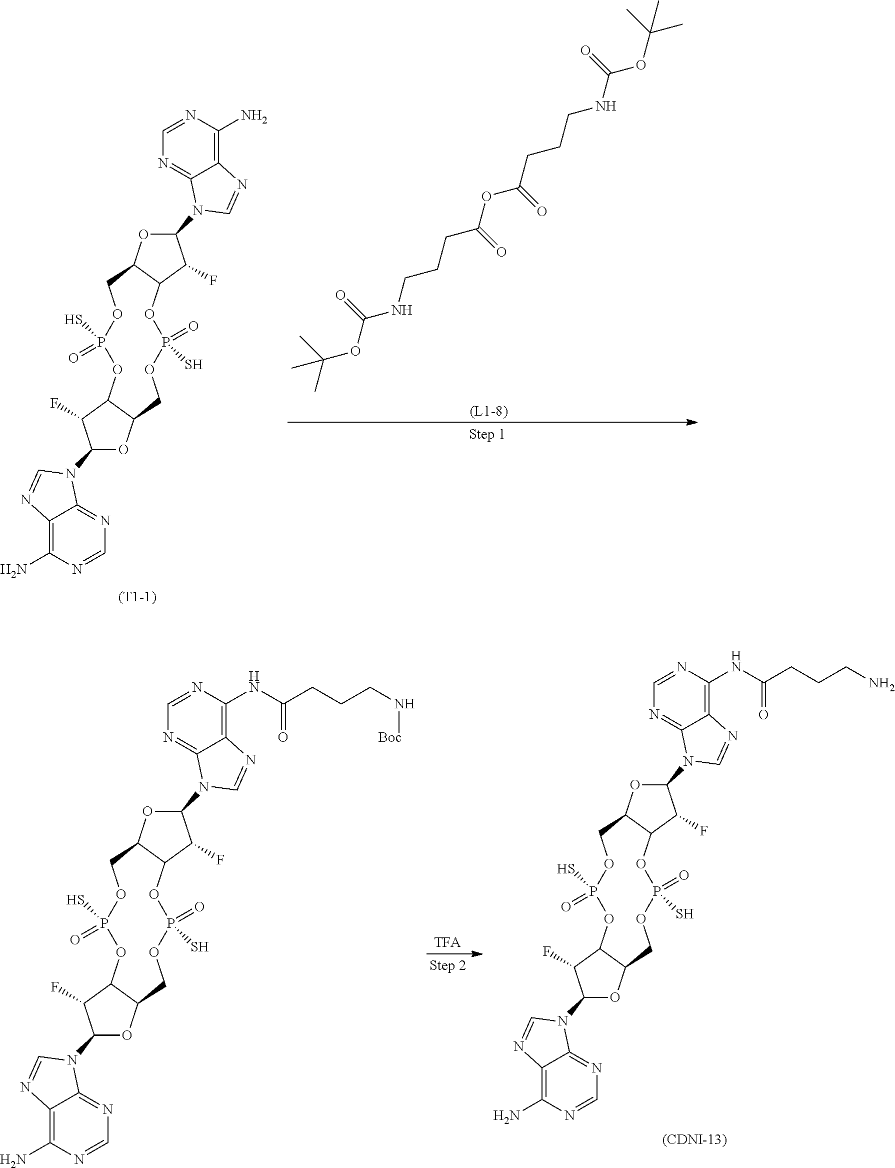

Step 1: 4-((tert-butoxycarbonyl)amino)butanoic anhydride (LI-8) was added to a solution of Compound (T1-1) Et₃N salt (30 mg, 0.033 mmol) in pyridine (5 ml) (390 mg, 1.00 mmol) and heated at 50° C. for 3 days. The reaction mixture was then concentrated and the crude was purified by reverse phase ISCO using 15 g C18 column, eluted with 5-60% acetonitrile-H₂O (aqueous phase containing 10 mM Et₃N HOAc). Fractions containing the desired product were isolated and concentrated to obtain boc-protected intermediate CDNI-13 as Et₃N salt (10 mg, 28% yield). LCMS M+1=880.1, tr=0.731 min.

Step 2: TFA (2 ml) was added to a flask containing boc-protected intermediate CDNI-12 Et₃N salt (10 mg, 0.009 mmol) and immediately concentrated. The crude was purified by reverse phase ISCO using 15 g C18 aq column, eluted with 5-60% acetonitrile-H₂O containing 0.05% TFA. Fractions containing the desired product were combined and lyophilized to obtain intermediate CDNI-13 as TFA salt (11.2 mg, 96% yield). LCMS M+1-H₂O=762.0, tr=0.608 min.

### Example 2-14: Synthesis of tert-butyl ((S)-1-((4-((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)amino)-4-oxobutyl)amino)-1-oxo-5-ureidopentan-2-yl)carbamate (CDNI-14)

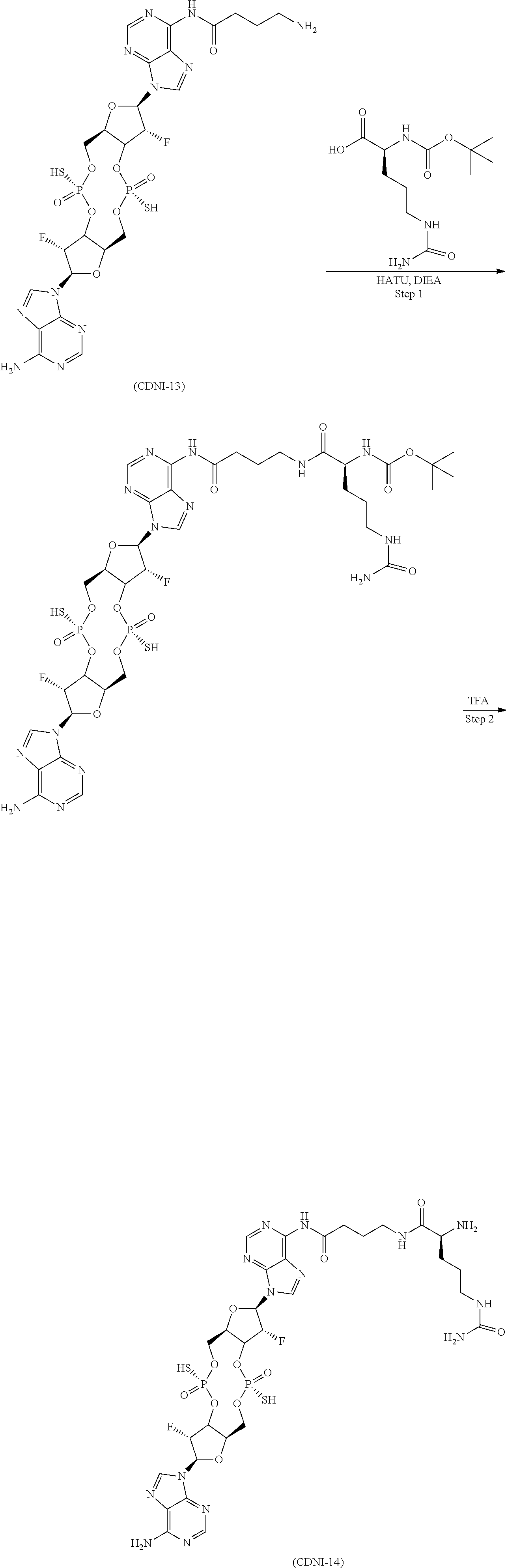

Step 1: To a solution of (S)-2-((tert-butoxycarbonyl)amino)-5-ureidopentanoic acid (Boc-Cit-OH purchased from Bachem) (2.7 mg, 0.01 mmol) in DMF (1 ml) was added DIEA (0.017 mL, 0.10 mmol) and then HATU (3.8 mg, 0.01 mmol). The reaction mixture was stirred at rt for 5 mins and then was added to a solution of CDN intermediate (CDNI-13) TFA salt (10 mg, 0.01 mmol) in DMF and this mixture was stirred at rt for 5 hrs and then concentrated. The residue was purified by reverse phase ISCO using 15 g C18 aq column, eluted with 5-40% acetonitrile-H₂O (aqueous phase containing 10 mM Et₃N HOAc). Fractions containing desired product were concentrated to obtain boc-protected intermediate CDNI-14 as an Et₃N salt (2.9 mg, 24% yield). LCMS M+1=1037.1, tr=0.699 min.

Step 2: TFA (1 ml) was added to a flask containing boc-protected intermediate CDNI-14 Et₃N salt (2.9 mg, 0.0028 mmol) and the solution was stirred for 1 min and then concentrated to give CDN intermediate (CDNI-14) as TFA salt (2.9 mg, 100% yield). LCMS M+1=937.1, tr=0.598 min.

### Example 2-15: Synthesis of (S)-2-((S)-2-amino-3-methylbutanamido)-N-(4-((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)amino)-4-oxobutyl)-5-ureidopentanamide (CDNI-15)

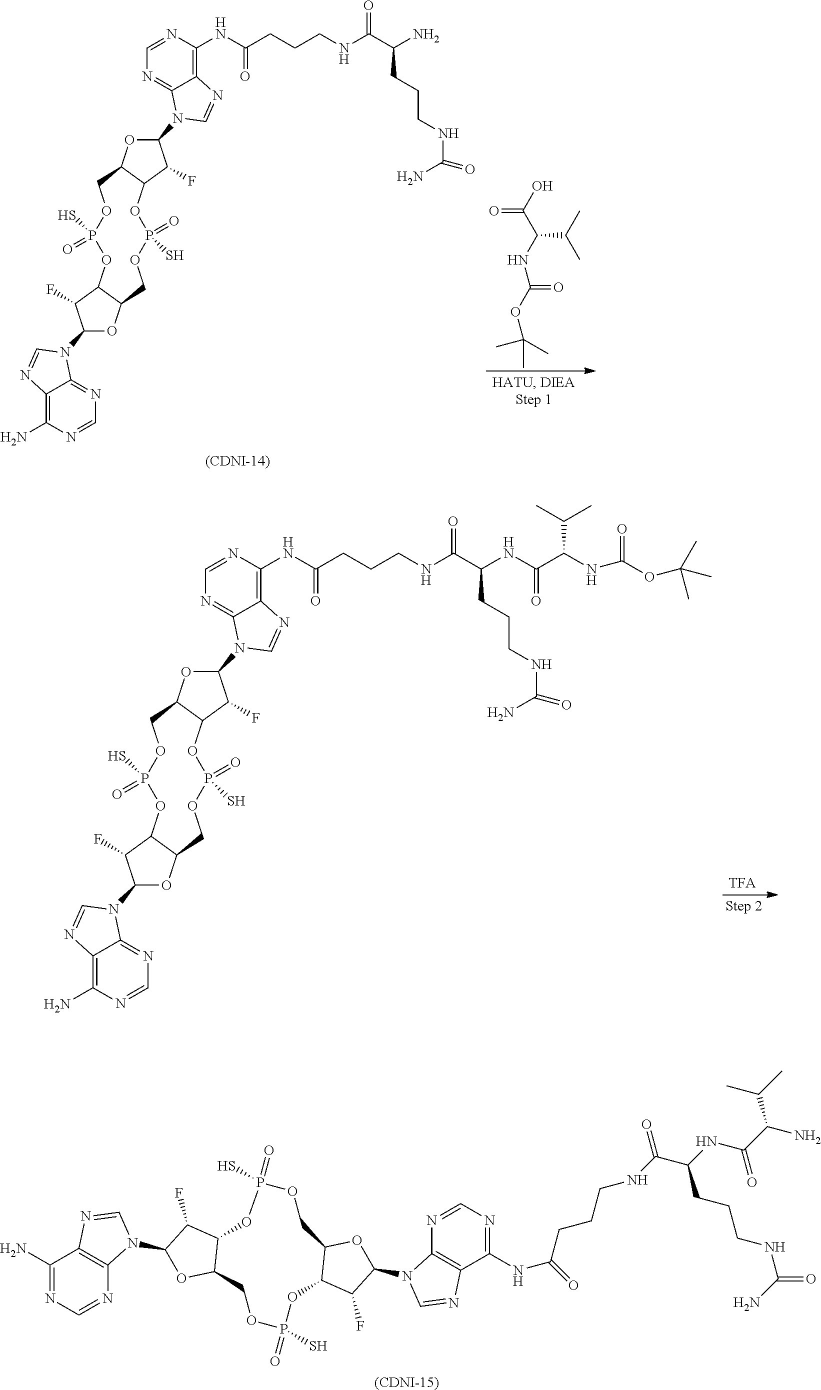

Step 1: To a vial containing (tert-butoxycarbonyl)-L-valine (Boc-Val-OH purchased from Novabiochem) (1.2 mg, 0.0056 mmol) was added DMF (1 ml) and then HATU (2.1 mg, 0.0056 mmol) and DIEA (3.6 mg, 0.028 mmol) were added. The mixture was stirred for 2 mins and then added to a solution containing intermediate CDNI-14 TFA salt (2.9 mg, 0.0028 mmol) in DMF (1 ml). The reaction was stirred at rt for 1 day and then concentrated. The residue was purified by reverse phase ISCO using 15 g C18 aq column, eluted with 5-40% acetonitrile-H₂O (aqueous phase containing 10 mM Et₃N HOAc). Fractions containing desired product were concentrated to obtain boc-protected intermediate CDNI-15 as Et₃N salt (1.8 mg, 48% yield). LCMS M+1=1136.2, tr=0.791 min.

Step 2: TFA (1 ml) was added to a flask containing boc-protected intermediate CDNI-15 Et₃N salt (1.8 mg, 0.0013 mmol) and the solution was stirred for 1 min and then concentrated to give intermediate CDNI-15 as TFA salt (1.7 mg, 100%). The compound was used in the next step without further purification. LCMS M+1=1036.1, tr=0.621 min.

### Example 2-16: Synthesis of (2S,3S,4S,5R,6S)-6-(4-((((2-(((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamoyl)oxy)ethyl)(methyl)carbamoyl)oxy)methyl)-2-(3-aminopropanamido)phenoxy)-3,4,5-trihydroxytetrahydro-2H-pyran-2-carboxylic acid (CDNI-16)

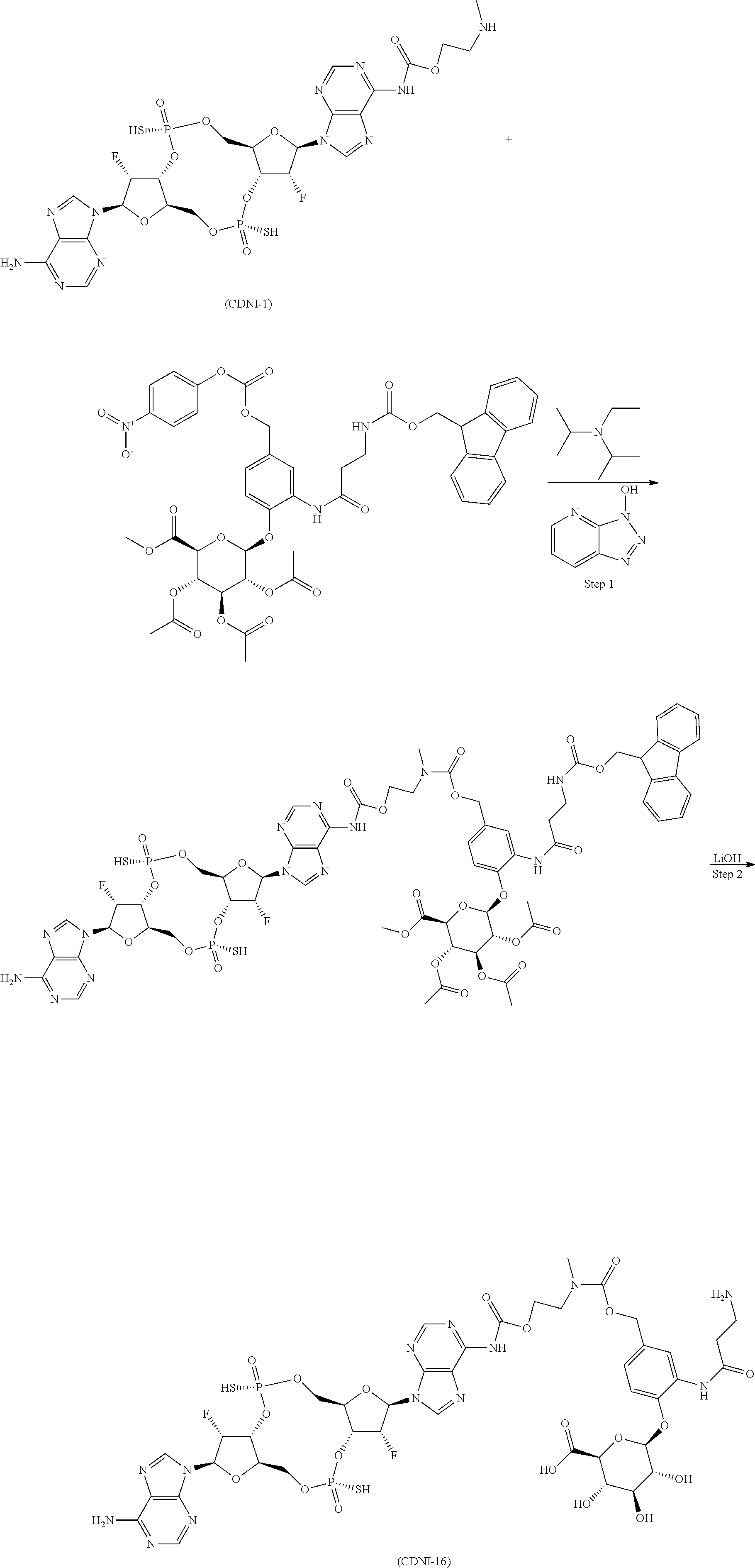

Step 1: To a solution of intermediate CDNI-1 TFA salt (15 mg, 0.015 mmol) and (2S,3R,4S,5S,6S)-2-(2-(3-((((9H-fluoren-9-yl) methoxy)carbonyl)amino)propanamido)-4-((((4-nitrophenoxy)carbonyl)oxy)methyl)phenoxy)-6-(methoxycarbonyl)tetrahydro-2H-pyran-3,4,5-triyl triacetate (see Bioconjugate Chem. 2006, 17, 831-840) (16 mg, 0.018 mmol) in DMF (1 ml) was added DIEA (0.026 ml, 0.15 mmol) and HOAT (2.0 mg, 0.015 mmol). The reaction was stirred at rt for 16 hrs. Solvent was then removed by high vacuum and the crude was purified by reverse phase ISACO using 15 g C18 column, eluted with 5-60% acetonitrile-H₂O (aqueous phase containing 10 mM Et₃N HOAc). Fractions containing desired product were concentrated to obtain Fmoc protected intermediate CDNI-16 as Et₃N salt (20.2 mg, 78% yield). LCMS M/2+1=785.8, tr=1.094 min.

Step 2: A solution of LiOH (9.3 mg, 0.388 mmol) in water was added to a vial containing Fmoc protected intermediate CDNI-16 (20.2 mg, 0.011 mmol) Et₃N salt and MeOH (4 mL) and the mixture was stirred at rt for 16 hrs. It was then neutralized with HOAc and concentrated. The crude was purified by reverse phase ISCO using 43 g C18 aq column, eluted with 5-35% acetonitrile-H₂O (aqueous phase containing 10 mM Et₃N HOAc). Fractions containing desired product were concentrated to obtain intermediate CDNI-16 as Et₃N salt (23.2 mg, 135% yield). LCMS M+1=1207.9, tr=0.811 min.

### Example 2-17: Synthesis of ((2S,4S)-4-fluoropyrrolidin-2-yl)methyl (9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamate (CDNI-17)

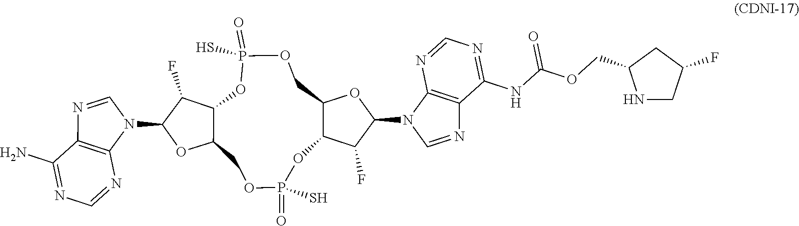 [SMILES: Nc1ncnc2c1ncn2[C@@H]1O[C@@H]2CO[P@@](=O)(S)O[C@H]3[C@@H](F)[C@H](n4cnc5c(NC(=O)OC[C@@H]6C[C@H](F)CN6)ncnc54)O[C@@H]3CO[P@@](=O)(S)O[C@H]2[C@H]1F]

Intermediate (CDNI-17) was synthesized using the method described for CDNI intermediate (CDNI-11), except Compound (T1-6) Et₃N salt was replaced with Compound (T1-1) Et₃N salt.

### Example 2-18: Synthesis of 2-azidoethyl (9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamate (CDNI-18)

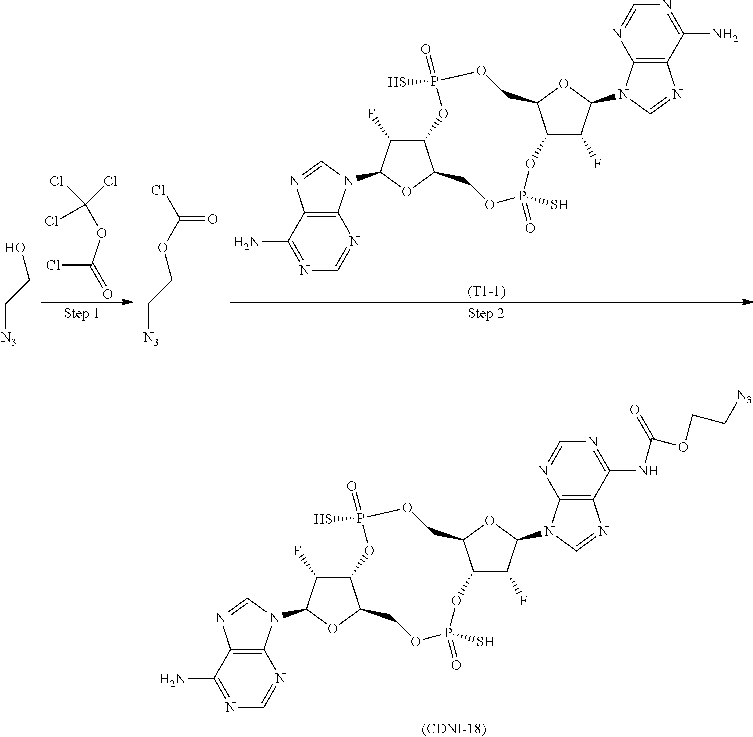

Step 1: A solution of diphosgene (275 mg, 1.41 mmol) was added to a solution of 2-azidoethanol (87 mg, 1.00 mmol) in DCM (10 ml) at −78° C. and the mixture was slowly warmed to rt. After 15 mins the solution became clear. The reaction was concentrated and solvent and other volatile reagents were removed under vacuum to obtain 2-azidoethyl carbonochloridate which was used in step 2 without further purification.

Step 2: 2-azidoethyl carbonochloridate (149 mg, 1.00 mmol) in DCM (1 ml) was added in portions over 30 mins to Compound (T1-1) Et₃N salt (30 mg, 0.033 mmol) dissolved in pyridine (2 ml). Then Et₃N (0.03 ml) was added and the mixture was stirred at rt for 2 hrs. The solution was concentrated and water and acetonitrile were then added. 1N NaOH (5 ml) was then added and the reaction was stirred at 60° C. for 2 hrs, Both mono- and diadduct were formed. The reaction was neutralized with HOAc, concentrated and then suspended in DMSO and purified by reverse phase ISCO using 43 g C18 aq column, eluted with 5-35%, acetonitrile-water (aqueous phase containing 10 mM Et₃N HOAc). Fractions containing mono-adduct were collected and concentrated to give CDNI intermediate (CDNI-18) as Et₃N salt (20 mg, 45% yield). LCMS M+1=808.0, tr=0.764 min.

### Example 2-19: Synthesis of N-(9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)-4-azidobutanamide (CDNI-19)

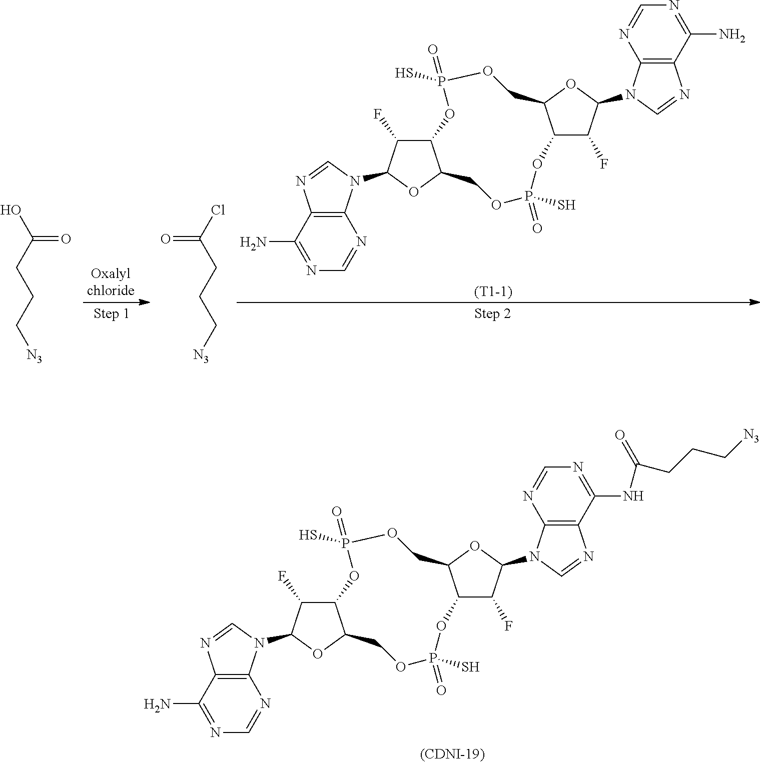 [SMILES: [N-]=[N+]=NCCCC(=O)O ; [N-]=[N+]=NCCCC(=O)Cl ; Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F ; "(T1-1)" ; [N-]=[N+]=NCCCC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F ; "(CDNI-19)"]

Step 1: 4-azidobutanoic acid (259 mg, 2.01 mmol) was dissolved in DCM (5 ml) and oxalyl chloride (190 mg, 1.5 mmol) was added, followed by DMF (0.005 ml). The reaction was stirred at rt for 1 hr, and then concentrated to obtain 4-azidobutanoyl chloride, which was used in the next step without further purification.

Step 2: 4-azidobutanoyl chloride (94 mg, 0.64 mmol) was dissolved in DCM (0.32 ml) and added to a solution of di-2′-F—RR-CDA Et₃N salt (30 mg, 0.033 mmol) in pyridine (3 ml). The reaction was stirred at 70° C. for 0.5 h and then quenched with 2 drops of water and concentrated. The crude was purified by reverse phase ISCO using 50 g C18 aq column, eluted with 5-50% acetonitrile-H₂O (aqueous phase containing 10 mM Et₃N HOAc). Fractions containing the desired product were isolated and lyophilized to give CDN intermediate (CDNI-19) as Et₃N salt (19.7 mg, 58.4% yield). LCMS M+1=806.0, tr=0.807 min.

### Example 2-20: Synthesis of 3-azidopropyl (9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamate (CDNI-20)

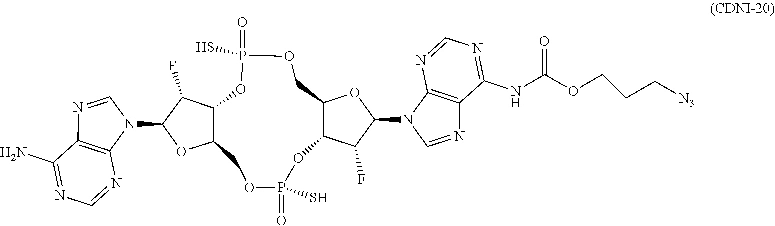 [SMILES: [N-]=[N+]=NCCCOC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F]

CDN intermediate (CDNI-20) was synthesized using the method described for the synthesis of CDN intermediate (CDNI-18) except 3-azidopropan-1-ol was used in place of 2-azidoethanol. CDN intermediate (CDNI-20) Et₃N salt (16.3 mg, 47% yield). LCMS M+1=822.0, tr=0.830 min.

### Example 2-21: Synthesis of a mixture of 4-amino-N-(9-((2R,3R,5S,7aR,9R,10R,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)butanamide (CDNI-21a) and N-(9-((2R,3R,5R,7aR,9R,10R,12S,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)-4-(methylamino)butanamide (CDNI-21b)

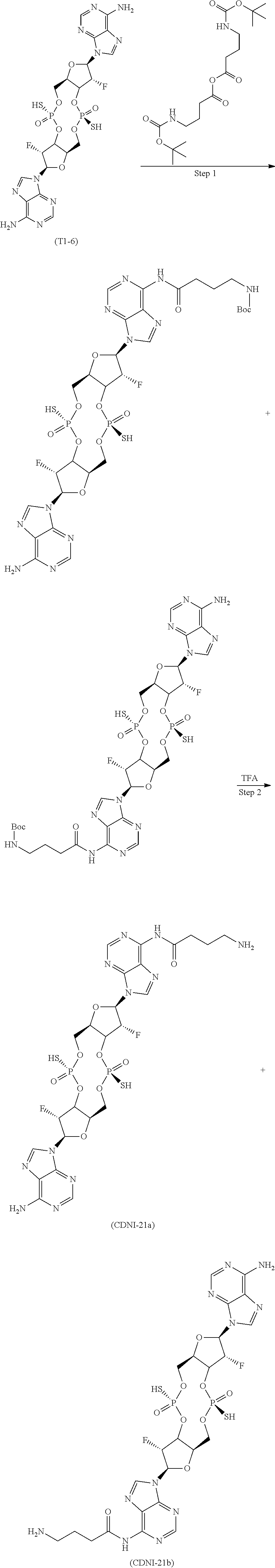

Step 1: NaH (60% dispersion in oil, 38.5 mg, 0.962 mmol) was added to a solution of Compound (T1-6) Et₃N salt (86.3 mg, 0.096 mmol) in DMF (3 ml) and the mixture was stirred for 1 min before the addition of 4-((tert-butoxycarbonyl)amino)butanoic anhydride (347 mg, 0.894 mmol). The reaction was stirred at rt for 1 hr and then quenched with HOAc (0.2 ml). The reaction was concentrated and purified using reverse phase ISCO with 15 g C18 aq column, eluted with 5-45% acetonitrile-H₂O (aqueous phase containing 10 mM Et₃N HOAc). Fractions containing desired product were collected and lyophilized to obtain boc protected CDN intermediate (CDNI-21a) and boc protected CDN intermediate (CDNI-21b) as Et₃N salt (20 mg, 19% yield). LCMS M+1=880.0, tr=0.782 min. The mixture was not separated. Note: 4-((tert-butoxycarbonyl)(methyl)amino)butanoic anhydride was synthesized as described in the synthesis of CDNI-4.

Step 2: To a flask containing boc protected CDN intermediate (CDNI-21a) and boc protected CDN intermediate (CDNI-21b) Et₃N salt (20 mg, 0.018 mmol) was added acetonitrile (5 ml) and TFA (1 ml) and the mixture was stirred for 30 mins and then concentrated. The residue was purified by reverse phase ISCO using 15 g C18 column, eluted with 5-50% acetonitrile-H₂O containing 0.05% TFA. Fractions containing desired product were concentrated to obtain a mixture of CDN intermediate (CDNI-21a) and CDN intermediate (CDNI-21b) as TFA salt (13.4 mg, 72% yield). LCMS M+1-H₂O=762, tr=0.268 min.

### Example 2-22: Synthesis of 4-((S)-2-((S)-2-amino-3-methylbutanamido)-5-ureidopentanamido)benzyl (2S,4S)-2-((((9-((2R,3R,3aR,5R,7aR,9R,10R,10aR,12S,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamoyl)oxy)methyl)-4-fluoropyrrolidine-1-carboxylate (CDNI-22a) and 4-((S)-2-((S)-2-amino-3-methylbutanamido)-5-ureidopentanamido)benzyl (2S,4S)-2-((((9-((2R,3R,3aR,5S,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamoyl)oxy)methyl)-4-fluoropyrrolidine-1-carboxylate (CDNI-22b)

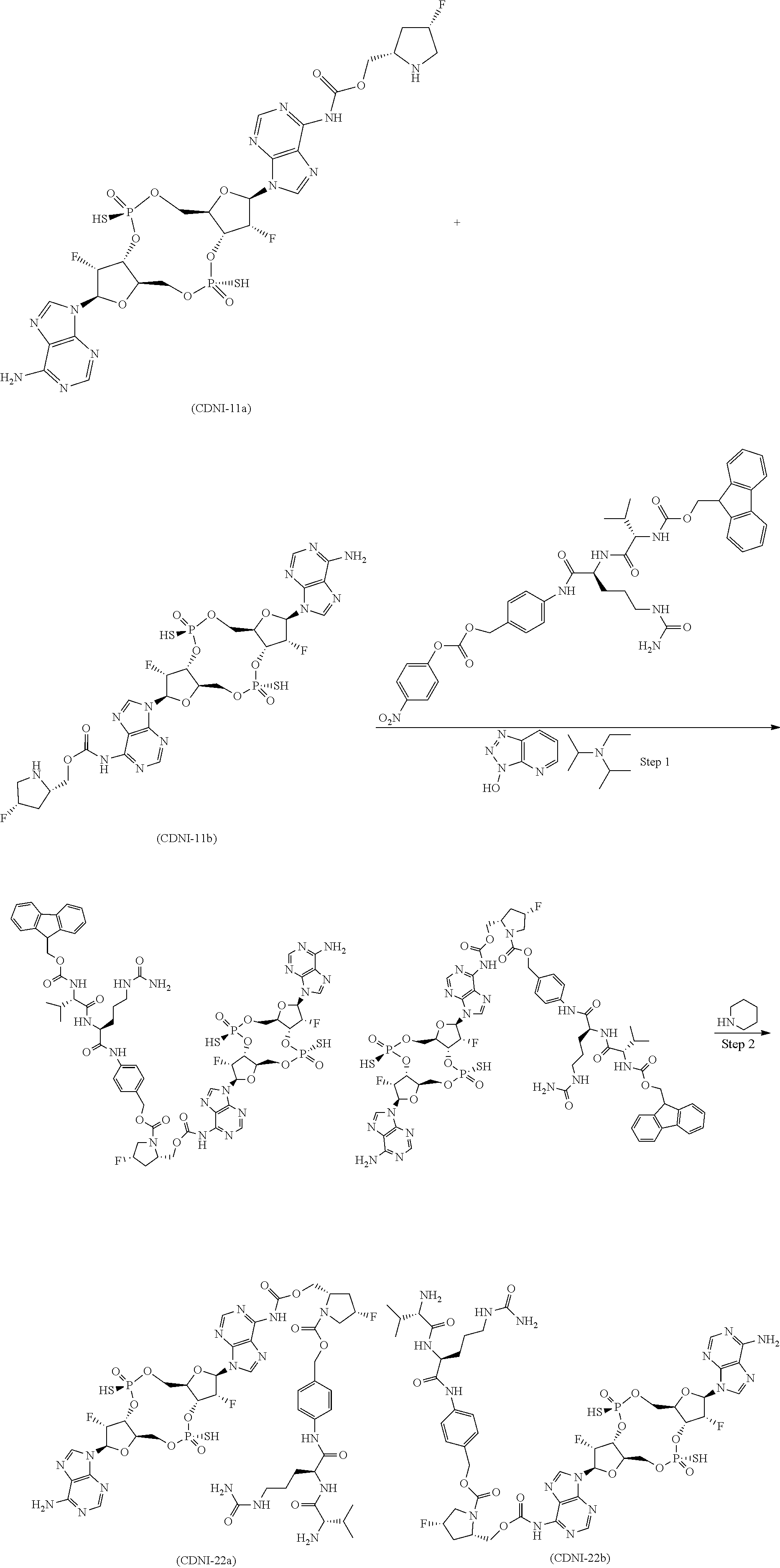

Step: Fmoc-Va-Cit-PABC-PNP (25.2 mg, 0.033 mmol) was added to a solution of CDN intermediate (CDNI-11a) and (CDNI-11b) (31.1 mg, 0.030 mmol) in DMF (1 ml), followed by the addition of DIEA (26.0 uL, 19.3 mg, 0.149 mmol) and HOAT (4.1 mg, 0.030 mmol). The reaction was stirred at rt overnight, water (1.0 mL) was then added and the solution concentrated. The residue was dissolved in DMSO and purified by ISCO by using 50.0 g C18 aq column, eluted with 5-60% ACN in water with 10 mM TEA-HOAc. Fractions containing desired product were concentrated to obtain compound Fmoc protected CDN intermediate (CDNI-22a and CDNI-22b) (42.2 mg, 80% yield) as TEA salt. LCMS M/2+1=734.30, tr=1.002 min.

Step 2: Piperidine (180.0 uL, 0.19 mmol) was added to a solution of Fmoc protected CDN intermediate (CDNI-22) (32.0 mg, 0.019 mmol) TEA salt in DMF (Volume: 3.0 mL) and the mixture was stirred at rt for 30 mins and then concentrated. The residue was purified by reverse phase ISCO 50 g C18 aq column, eluted with 5-35% acetonitrile-H₂O containing 0.05% TFA. Fractions containing desired product were concentrated to obtain CDN intermediate (CDNI-22a and CDN22b) (20.0 mg, 67.8%) as TFA salt. LCMS M/2+1=623.3, tr=0.790 min.

### Example 2-23: Synthesis of 2-(methylamino)ethyl (9-((1S,3R,6R,8R,9S,11R,14R,16R,17R,18R)-16-(6-amino-9H-purin-9-yl)-17,18-difluoro-3,11-dimercapto-3,11-dioxido-2,4,7,10,12,15-hexaoxa-3,11-diphosphatricyclo[12.2.1.16,9]octadecan-8-yl)-9H-purin-6-yl)carbamate (CDNI-23)

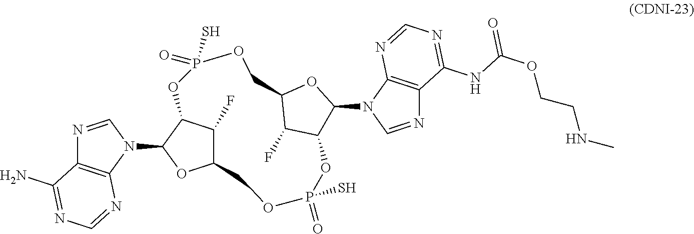 [SMILES: CNCCOC(=O)Nc1ncnc2c1ncn2[C@@H]1O[C@@H]2CO[P@@](=O)(S)O[C@@H]3[C@H](F)[C@@H](CO[P@@](=O)(S)O[C@@H]1[C@@H]2F)O[C@H]3n1cnc2c(N)ncnc21]

CDN intermediate (CDNI-23) was synthesized using the method described for the synthesis of CDN intermediate (CDNI-1) except Compound (T1-1) Et₃N salt was replaced with Compound (T2-46) Et₃N salt.

Boc-protected CDN intermediate (CDNI-23): LCMS M+1=796.0, tr=0.625 min. ¹H NMR (500 MHz, DMSO-d₆) δ 10.71 (s, 1H), 9.36 (d, J=6.1 Hz, 2H), 8.92 (s, 1H), 8.73 (s, 2H), 8.39 (s, 1H), 6.27 (dd, J=44.7, 8.4 Hz, 2H), 5.79-5.33 (m, 4H), 4.75-4.55 (m, 3H), 4.38 (s, 1H), 4.00 (dd, J=12.5, 5.4 Hz, 4H), 3.35 (dd, J=10.3, 6.4 Hz, 1H), 3.25 (s, 1H), 3.12 (tt, J=7.4, 3.7 Hz, 1H).

CDN intermediate (CDNI-23) TFA salt (8.2 mg, 55.0% yield). LCMS M+1=796.0, tr=0.625 min. ¹H NMR (500 MHz, DMSO-d₆) δ 10.71 (s, 1H), 9.36 (d, J=6.1 Hz, 2H), 8.92 (s, 1H), 8.73 (s, 2H), 8.39 (s, 1H), 6.27 (dd, J=44.7, 8.4 Hz, 2H), 5.79-5.33 (m, 4H), 4.75-4.55 (m, 3H), 4.38 (s, 1H), 4.00 (dd, J=12.5, 5.4 Hz, 4H), 3.35 (dd, J=10.3, 6.4 Hz, 1H), 3.25 (s, 1H), 3.12 (tt, J=7.4, 3.7 Hz, 1H).

### Example 2-24: Synthesis of (2R,3R,3aR,5R,7aR,9R,10R,10aR,12S,14aR)-2-(2-amino-6-oxo-1,6-dihydro-9H-purin-9-yl)-9-(6-amino-9H-purin-9-yl)-10-fluoro-5,12-dimercapto-5,12-dioxidooctahydro-2H,7H-difuro[3,2-d:3′,2′-j][1,3,7,9]tetraoxa[2,8]diphosphacyclododecin-3-yl (2-(methylamino)ethyl) carbonate (CDNI-24)

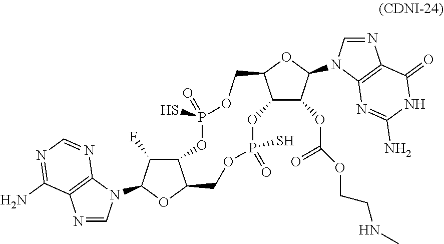 [SMILES: CNCCOC(=O)O[C@@H]1[C@@H]2O[P@](=O)(S)OC[C@H]3O[C@@H](n4cnc5c(N)ncnc54)[C@H](F)[C@@H]3O[P@@](=O)(S)OC[C@H]2O[C@H]1n1cnc2c(=O)[nH]c(N)nc21]

CDN intermediate (CDNI-24) was synthesized using the method described for the synthesis of CDN intermediate (CDNI-3) except Compound (T1-2) Et₃N salt was replaced with Compound (T1-13) Et₃N salt.

Boc-protected CDN intermediate (CDNI-24): LCMS M+1=910.1, tr=0.731 min. ¹H NMR (500 MHz, Methanol-d₄) δ 8.46 (s, 1H), 8.20 (d, J=7.6 Hz, 2H), 6.36 (d, J=17.1 Hz, 1H), 6.07 (d, J=11.8 Hz, 1H), 5.77-5.56 (m, 2H), 5.34 (s, 1H), 5.24-5.04 (m, 1H), 4.60 (dt, J=12.3, 2.7 Hz, 1H), 4.42 (d, J=10.2 Hz, 3H), 4.32 (d, J=8.0 Hz, 3H), 4.08-3.95 (m, 2H), 3.64 (t, J=5.9 Hz, 5H), 3.58 (s, 2H), 3.03 (q, J=7.3 Hz, 31H), 2.96 (s, 4H), 2.92 (s, 9H), 1.22 (t, J=7.3 Hz, 42H).

CDN intermediate (CDNI-24) TFA salt (8.1 mg, 71.7% yield). LCMS M+1=810.2, tr=0.346 min. ¹H NMR (500 MHz, DMSO-d₆) δ 10.80 (s, 1H), 9.36 (d, J=42.0 Hz, 2H), 8.48 (d, J=45.8 Hz, 2H), 8.27 (s, 1H), 6.70 (s, 2H), 6.41 (d, J=16.4 Hz, 1H), 6.06 (d, J=7.3 Hz, 1H), 5.70-5.38 (m, 2H), 5.16 (dtd, J=26.2, 9.3, 4.6 Hz, 1H), 4.90 (ddd, J=11.5, 5.4, 2.9 Hz, 1H), 4.59 (ddd, J=12.9, 6.7, 2.4 Hz, 1H), 4.40 (dd, J=11.4, 5.3 Hz, 2H), 4.26 (ddd, J=17.0, 8.5, 5.9 Hz, 1H), 4.23-4.06 (m, 1H), 3.92-3.71 (m, 2H), 3.43-3.17 (m, 2H), 3.13 (td, J=7.3, 4.8 Hz, 1H), 2.67 (t, J=5.2 Hz, 3H).

### Example 2-25: Synthesis (2R,3R,3aR,5R,7aR,9R,10R,10aS,12R,14aR)-2,9-bis(2-amino-6-oxo-1,6-dihydro-9H-purin-9-yl)-10-hydroxy-5,12-dimercapto-5,12-dioxidooctahydro-2H,7H-difuro[3,2-d:3′,2′-j][1,3,7,9]tetraoxa[2,8]diphosphacyclododecin-3-yl (2-(methylamino)ethyl) carbonate (CDNI-25)

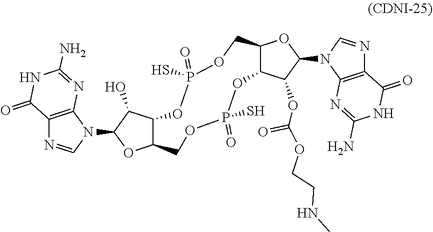 [SMILES: CNCCOC(=O)O[C@@H]1[C@@H]2O[P@](=O)(S)OC[C@H]3O[C@@H](n4cnc5c(=O)[nH]c(N)nc54)[C@H](O)[C@@H]3O[P@](=O)(S)OC[C@H]2O[C@H]1n1cnc2c(=O)[nH]c(N)nc21]

CDN intermediate (CDNI-24) was synthesized using the method described for the synthesis of CDN intermediate (CDNI-3) except Compound (T1-2) Et₃N salt was replaced with Compound (T1-16) Et₃N salt.

Boc-protected CDN intermediate (CDNI-25): LCMS M+1=924.2. tr=0.813 min.

CDN intermediate (CDNI-25) TFA salt (5.9 mg, 46.2% yield). LCMS M+1=824.0 tr=0.410 min. ¹H NMR (500 MHz, DMSO-d₆) δ 10.64 (d, J=12.1 Hz, 1H), 9.26 (d, J=105.9 Hz, 1H), 8.04 (d, J=5.7 Hz, 1H), 6.59 (s, 2H), 5.96 (d, J=7.8 Hz, 1H), 5.80-5.61 (m, 1H), 4.81 (ddd, J=72.1, 9.8, 4.4 Hz, 1H), 4.57-4.43 (m, 1H), 4.29-3.88 (m, 3H), 3.28-2.97 (m, 1H.

### Example 2-26: Synthesis ((2R,3R,3aR,5R,7aR,9R,10R,10aR,12S,14aR)-2,9-bis(6-amino-9H-purin-9-yl)-10-fluoro-5,12-dimercapto-5,12-dioxidooctahydro-2H,7H-difuro[3,2-d:3′,2′-j][1,3,7,9]tetraoxa[2,8]diphosphacyclododecin-3-yl D-prolinate (CDNI-26)

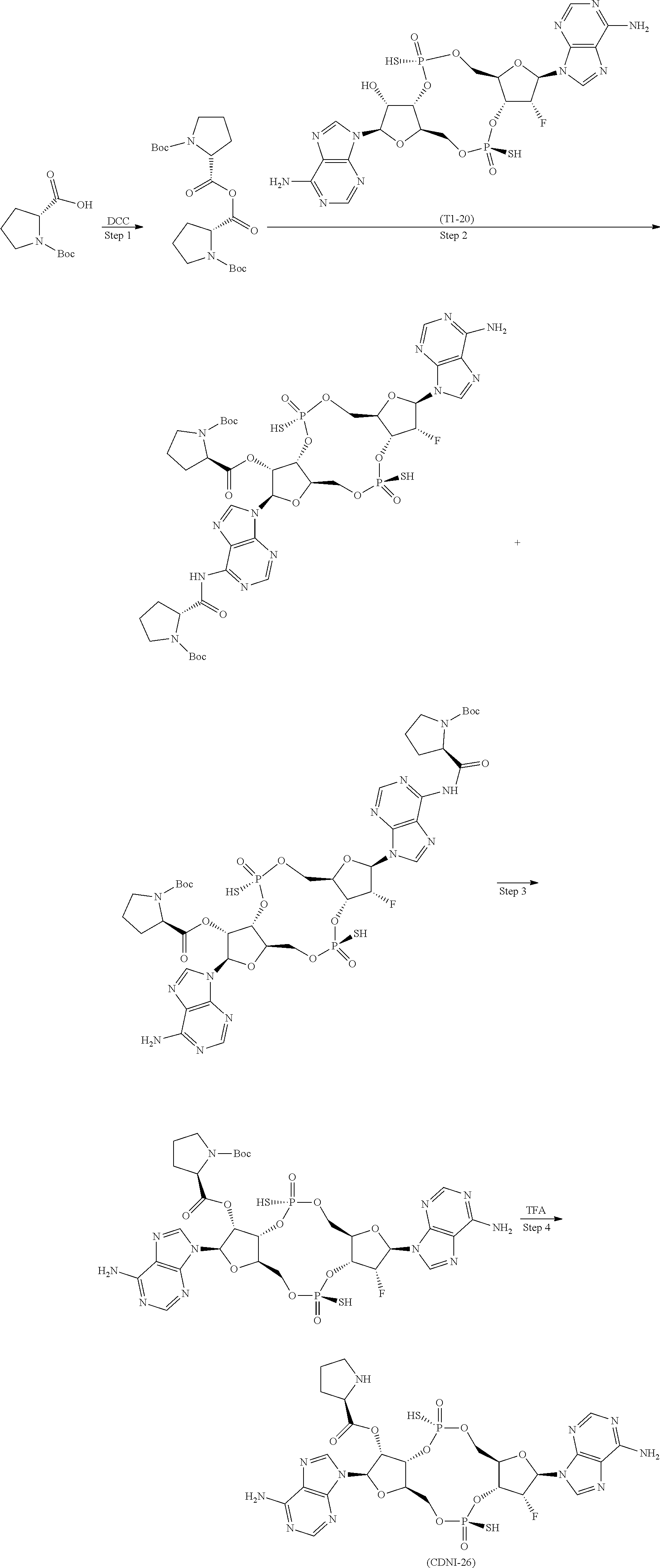

Step 1: A solution of dicyclohexylcarbodiimide (0.51 eq) in 5 ml of anhydrous DCM is added under nitrogen drop wise, with stirring, to a solution of (R)-1-(tert-butoxycarbonyl)pyrrolidine-2-carboxylic acid (purchased from Combi-Blocks) (2.152 g, 10 mmol) in anhydrous dichloromethane (45 ml). The solution was stirred for 150 min and the resulting urea precipitate was removed by filtration and the filtrate was concentrated to about 5 ml, and then filtered through syringe filter. The solvent was removed under vacuum to give (R)-1-(tert-butoxycarbonyl)pyrrolidine-2-carboxylic anhydride as a sticky oil (2.169 g, 100% yield).

Step 2: (R)-1-(tert-butoxycarbonyl)pyrrolidine-2-carboxylic anhydride (501 mg, 1.117 mmol) in NMP (3 mL) was added to Compound (T1-20) sodium salt (55 mg, 0.074 mmol) in pyridine (1.5 mL) and the mixture was stirred at rt for two days. n-Butylamine (0.1 mL) in water (1.0 mL) was then added and the mixture was stirred at rt for 10 mins. The pyridine and water were then removed under vacuum and the NMP was removed by lyophilization. The crude was purified by reverse phase ISCO using 50 g C18 aq column, eluted with 5-55% acetonitrile-H₂O (aqueous phase containing 10 mM Et₃N HOAc) to the boc-protected diadducts of CDN intermediate (CDNI-26). All diadducts were collected, dried by lyophilization.

Step 3: The boc-protected diadduct was dissolved in MeOH (5 mL) in a 30 mL pressure vessel equipped with a Teflon valve. The vessel was placed in an oil bath heated at 110° C. for 5 hours. Volatiles were evaporated, and the residues was purified by reverse phase ISCO using 50 g C18 aq. column, eluted with 5-55% acetonitrile-H₂O (aqueous phase containing 10 mM Et₃N HOAc). Fractions containing the desired product were combined and lyophilized to obtain boc-protected CDN intermediate (CDNI-26) as Et₃N salt (18.9 mg). LCMS M+1=890.0, tr=0.722 min.

Step 4: To a vial containing boc-protected CDN intermediate (CDNI-26) Et₃N salt (30.0 mg, 0.034 mmol) was added TFA (2 ml). The mixture was concentrated immediately and then concentrated. The crude was purified by reverse phase ISCO using 50 g C18 column, eluted with 5-40% acetonitrile-H₂O (aqueous phase containing 10 mM Et₃N HOAc). Fractions containing desired product were concentrated to obtain CDN intermediate (CDNI-26) as TEA salt (12.4 mg, 37.1% yield). LCMS M+1=790.1, tr=0.350 min.

### Example 2-27: Synthesis of a mixture of CDN intermediate (CDNI-27a) and CDN intermediate (CDNI-27b)

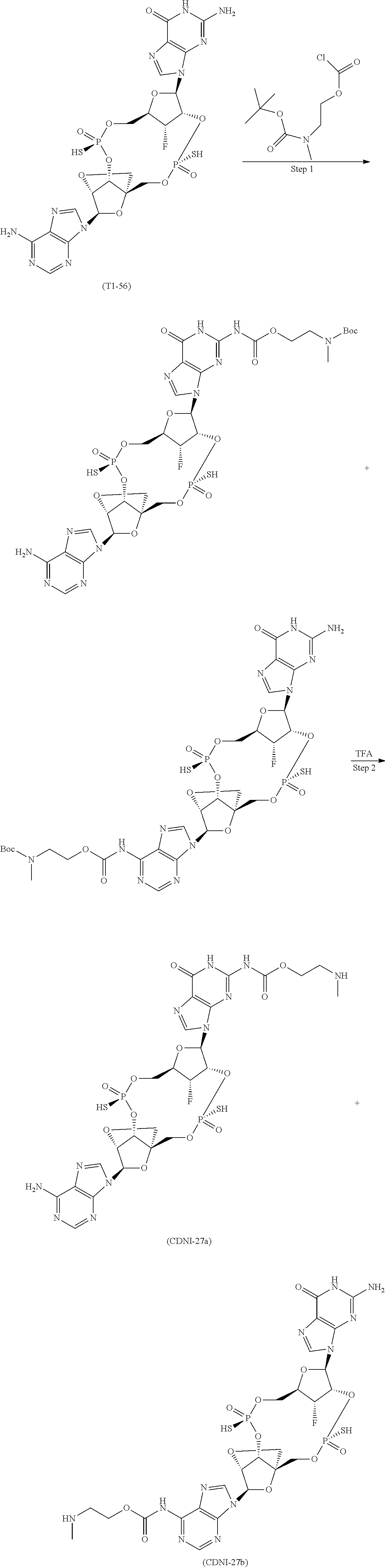

The mixture of CDN Intermediate (CDNI-27a) and CDN Intermediate (CDNI-27b) was prepared using the methods described for the synthesis of intermediate (CDNI-3), except Compound (T1-56) was used in place of Compound (T1-2), the reaction mixture of step was stirred for 2 hours instead of 30 mins and in step 1 purification used 5-50% acetonitrile-H₂O (aqueous phase containing 10 mM Et₃N HOAc).

CDN Intermediate (CDNI-27a) and CDN Intermediate (CDNI-27b) as TEA salt (3.7 mg, 55.9% yield). LCMS M+1=822.0, tr=0.319 min.

Note: The mixture was not separated and 2-((tert-butoxycarbonyl)(methyl)amino)ethyl carbonochloridate was synthesized as described in the synthesis of CDNI-9 except the initial temperature was −30° C. instead of −15° C.

### Example 2-28: Synthesis (2R,3R,3aR,5S,7aR,9R,10R,10aR,12R,14aR)-9-(2-amino-6-oxo-1,6-dihydro-9H-purin-9-yl)-2-(6-amino-9H-purin-9-yl)-10-fluoro-5,12-dimercapto-5,12-dioxidooctahydro-2H,7H-difuro[3,2-d:3′,2′-j][1,3,7,9]tetraoxa[2,8]diphosphacyclododecin-3-yl (2-(methylamino)ethyl) carbonate (CDNI-28)

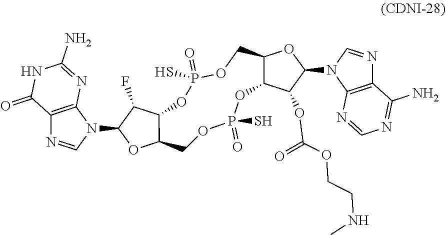 [SMILES: CNCCOC(=O)O[C@@H]1[C@@H]2O[P@@](=O)(S)OC[C@H]3O[C@@H](n4cnc5c(=O)[nH]c(N)nc54)[C@H](F)[C@@H]3O[P@](=O)(S)OC[C@H]2O[C@H]1n1cnc2c(N)ncnc21]

CDN intermediate (CDNI-28) was synthesized using the method described for the synthesis of CDN intermediate (CDNI-3) except Compound (T1-2) Et₃N salt was replaced with Compound (T1-11) Et₃N salt, the reaction time in Step 2 was 2 hrs rather than 30 mins and purification of CDN intermediate (CDNI-28) was by reverse phase ISCO using 15 g C18 column, eluted with 5-40% acetonitrile-H₂O (aqueous phase containing 10 mM Et₃N HOAc).

Boc-protected CDN intermediate (CDNI-28) as Et₃N salt (8.9 mg, 52.1% yield). LCMS M+1=910.1. tr=0.731 min.

CDN intermediate (CDNI-28) as TEA salt (6.5 mg, 62.4% yield). LCMS M+1=810.0 tr=0.350 min.

### Example 2-29: Synthesis (2R,3R,3aR,7aR,9R,10R,10aR,14aR)-2-(6-((3-amino-2-hydroxypropyl)amino)-9H-purin-9-yl)-9-(6-amino-9H-purin-9-yl)-3,10-difluoro-5,12-dihydroxyoctahydro-2H,7H-difuro[3,2-d:3′,2′-j][1,3,7,9]tetraoxa[2,8]diphosphacyclododecine 5,12-dioxide (CDNI-29)

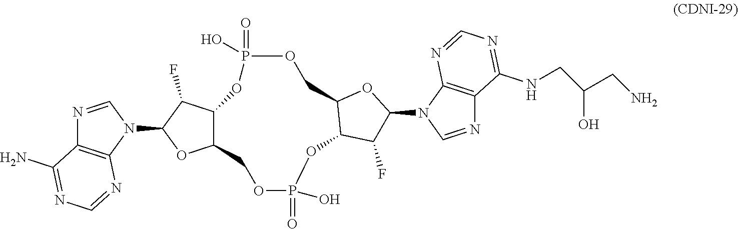 [SMILES: NCC(O)CNc1ncnc2c1ncn2[C@@H]1O[C@@H]2COP(=O)(O)O[C@H]3[C@@H](F)[C@H](n4cnc5c(N)ncnc54)O[C@@H]3COP(=O)(O)O[C@H]2[C@H]1F]

Step 1: To a solution of Compound (T1-1) Et₃N salt (30 mg, 0.033 mmol) in DMF (3 ml) was added tert-butyl (oxiran-2-ylmethyl)carbamate (57.9 mg, 0.334 mmol) and DIEA (43.2 mg, 0.334 mmol). The mixture was heated to 100° C. for 4 hours and the solvent was removed. The crude product was purified by reverse phase ISCO using 50 g C18 aq column, eluted with 5-45% acetonitrile-H₂O (aqueous phase containing 10 mM Et₃N HOAc). Fractions containing boc protected CDN intermediate (CDNI-29) were isolated and lyophilized to obtain Boc protected CDN intermediate (CDNI-29) as Et₃N salt (20 mg, 58% yield). LCMS M+1=836.0, tr=0.538 min.

Step 2: To a 25 ml round-bottom flask containing boc protected CDN intermediate (CDNI-29) Et₃N salt (20 mg, 0.019 mmol) was added TFA (1 ml, 13 mmol). The mixture was stirred for 1 min and then concentrated. The residue was purified by reverse phase ISCO using 50 g C18 aq column, eluted with 5-35% acetonitrile-H₂O (aqueous phase containing 10 mM Et₃N HOAc). Fractions containing desired product were concentrated to obtain CDN intermediate (CDNI-29) as Et₃N salt (11.1 mg, 62% yield). LCMS M+1=736.0, tr=0.235 min.

### Example 3: Synthesis of Exemplary Linker-Drug Compounds

### Example 3-1: Synthesis of Compound 12 (C12)

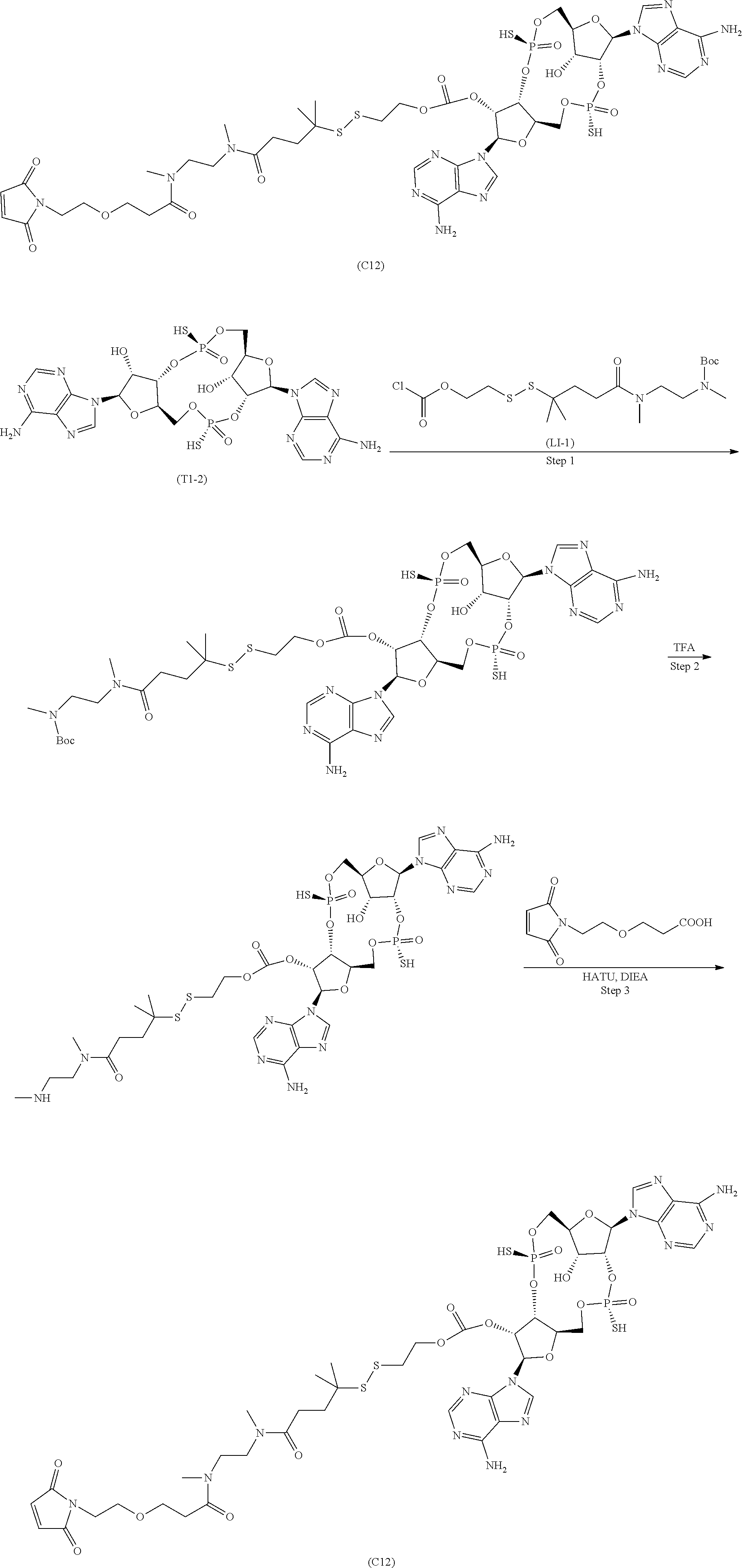

Step 1:

Compound (T1-2) (5 mg, 0.007 mmol) disodium salt was dissolved in anhydrous pyridine (1 ml) followed by the addition of Et₃N (0.005 ml). The mixture was sonicated and then linker intermediate (LI-1) (30 mg, 0.068 mmol) was added. The reaction mixture was stirred for 30 mins at room temperature and monitored by LCMS. The mixture was concentrated and then dissolved in MeOH-water, followed by purification by mass triggered reverse phase HPLC, using C18 column, eluted with 5-55% acetonitrile-H₂O containing 0.05% TFA. Fractions containing the desired boc-protected carbonate (2 mg, 22%) were collected LCMS M+1=1111.1, tr=0.898 min.

Step 2.

TFA (1 ml) was added to a vial containing the carbonate from step 1 (2 mg, 0.0015 mmol) and then immediately concentrated. The residue was then dissolved in MeOH and purified by ISCO using Ig C18 column, eluted with 5-50% ACN-water containing 0.05% TFA. Fractions containing the desired product were combined and lyophilized to give the de-protected carbonate (1.0 mg, 11% yield) as TFA salt. LCMS M/2+1=506.2, tr=0.669 min.

Step 3.

DIEA (15 mg, 0.116 mmol) and then HATU (3.4 mg, 0.0089 mmol) were added to a solution of 3-(2-(2,5-dioxo-2,5-dihydro-1H-pyrrol-1-yl)ethoxy)propanoic acid (Mal-PEG1-Acid) (1.9 mg, 0.0089 mmol) in DMF (1 ml) and the reaction mixture was stirred at room temperature for 5 mins. 10% of this reaction mixture was then added to a flask containing the de-protected carbonate obtained in step 2 (1.0 mg, 0.00089 mmol) in 0.5 ml DMF. The reaction was stirred at room temperature for 2 hours and then purified by mass-triggered reverse phase HPLC, using C18 column, eluted with 5-37% acetonitrile-H₂O containing 0.05% TFA. The fractions containing desired product were concentrated to obtain Compound (C12) (0.7 mg, 57% yield) as TFA salt. LCMS M+1=1206.3, M/2+1=603.7, tr=0.784 min.

### Example 3-2: Synthesis of Compound 13 (C13)

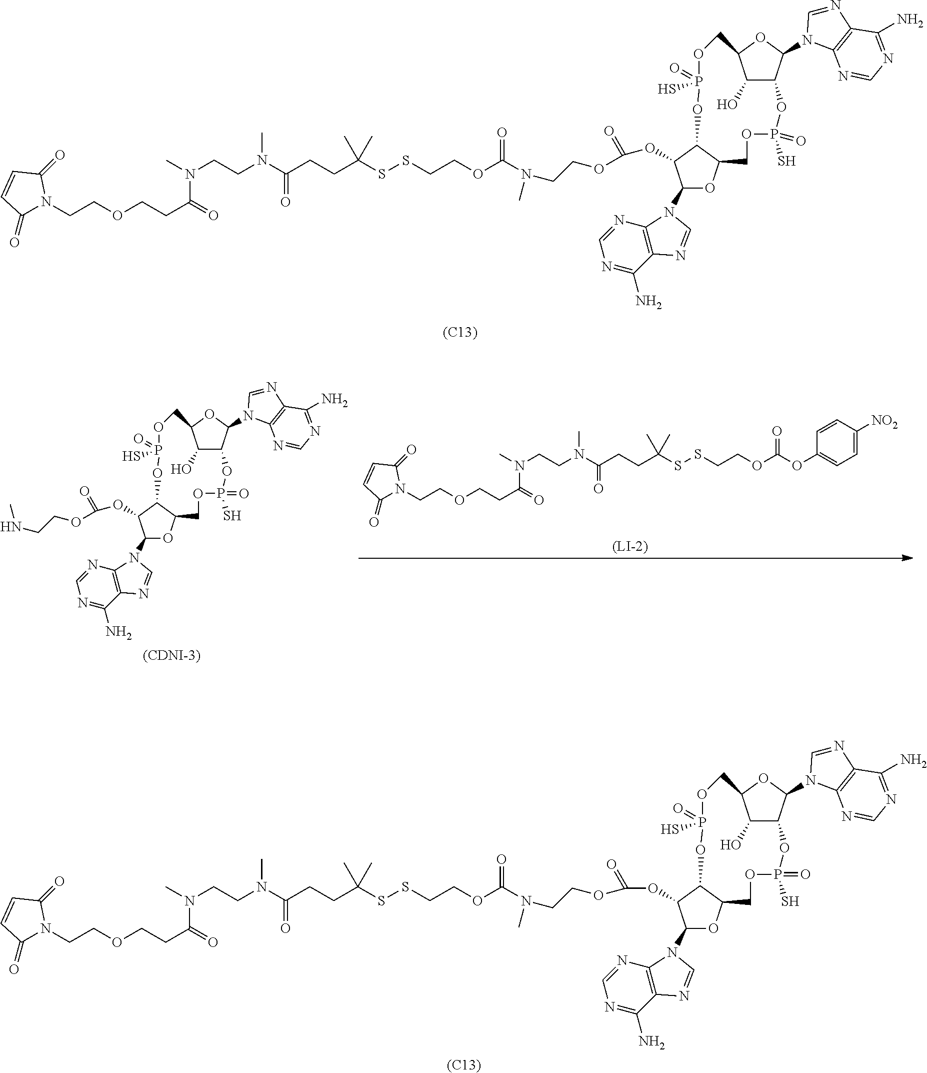

18-(2,5-dioxo-2,5-dihydro-1H-pyrrol-1-yl)-5,5,9,12-tetramethyl-8,13-dioxo-16-oxa-3,4-dithia-9,12-diazaoctadecyl (4-nitrophenyl) carbonate (LI-2) (2.5 mg, 0.0039 mmol) and DIEA (0.013 mmol) in DMF (1 ml) and the mixture was stirred at room temperature for 5 hours. The crude was purified by mass-triggered reverse phase HPLC, using C18 column, eluted with 20-33% acetonitrile-H2O containing 0.05% TFA. Fractions containing desired product were concentrated to compound A2 (2.2 mg, 38.1% yield) as TFA salt. LCMS M/2+1=654.2, tr=0.799 min.

### Example 3-3: Synthesis of Compound 14 (C14)

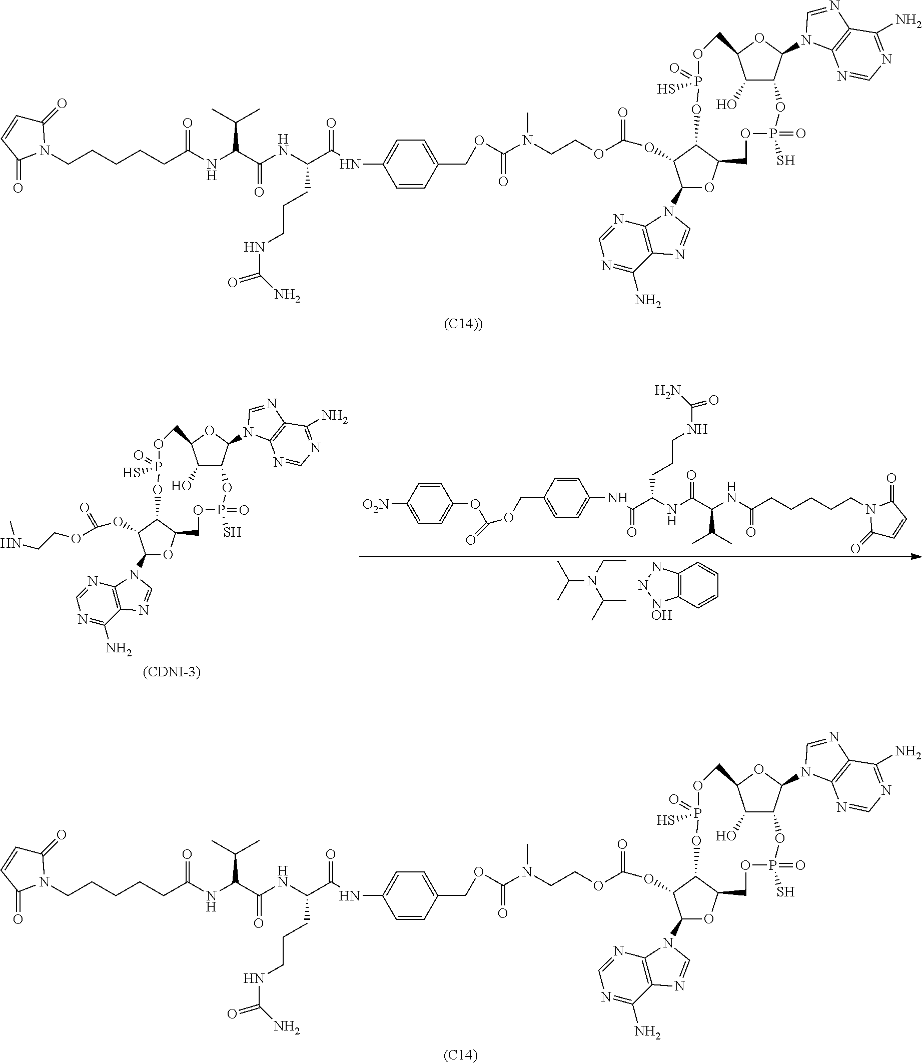

CDN intermediate (CDNI-3) ((7.4 mg, 0.0073 mol) TFA salt was dissolved in anhydrous DMF (2 ml) and 4-((S)-2-((S)-2-(6-(2,5-dioxo-2,5-dihydro-1H-pyrrol-8,13-yl)hexanamido)-3-methylbutanamido)-5-ureidopentanamido)benzyl (4-nitrophenyl) carbonate (MC-vc-pab PNP purchased from Levena Biopharma, San Diego) (6.3 mg, 0.009 mmol) was added, followed by addition of DIEA (11 mg, 0.084 mmol) and HOAT (4 mg, 0.029 mmol). The mixture was stirred at room temperature for 3 days and monitored by LCMS until completion of the reaction. The mixture was then purified by mass triggered reverse phase HPLC, using C18 column, eluted with 5-35% acetonitrile-H₂O containing 0.05% TFA. Fractions containing the desired product were combined and concentrated to obtain Compound (C14) (3.6 mg, 25.8% yield) as a TFA salt. LCMS M/2+1=695.8, tr=0.783 min.

### Example 3-4: Synthesis of Compound 15 (C15)

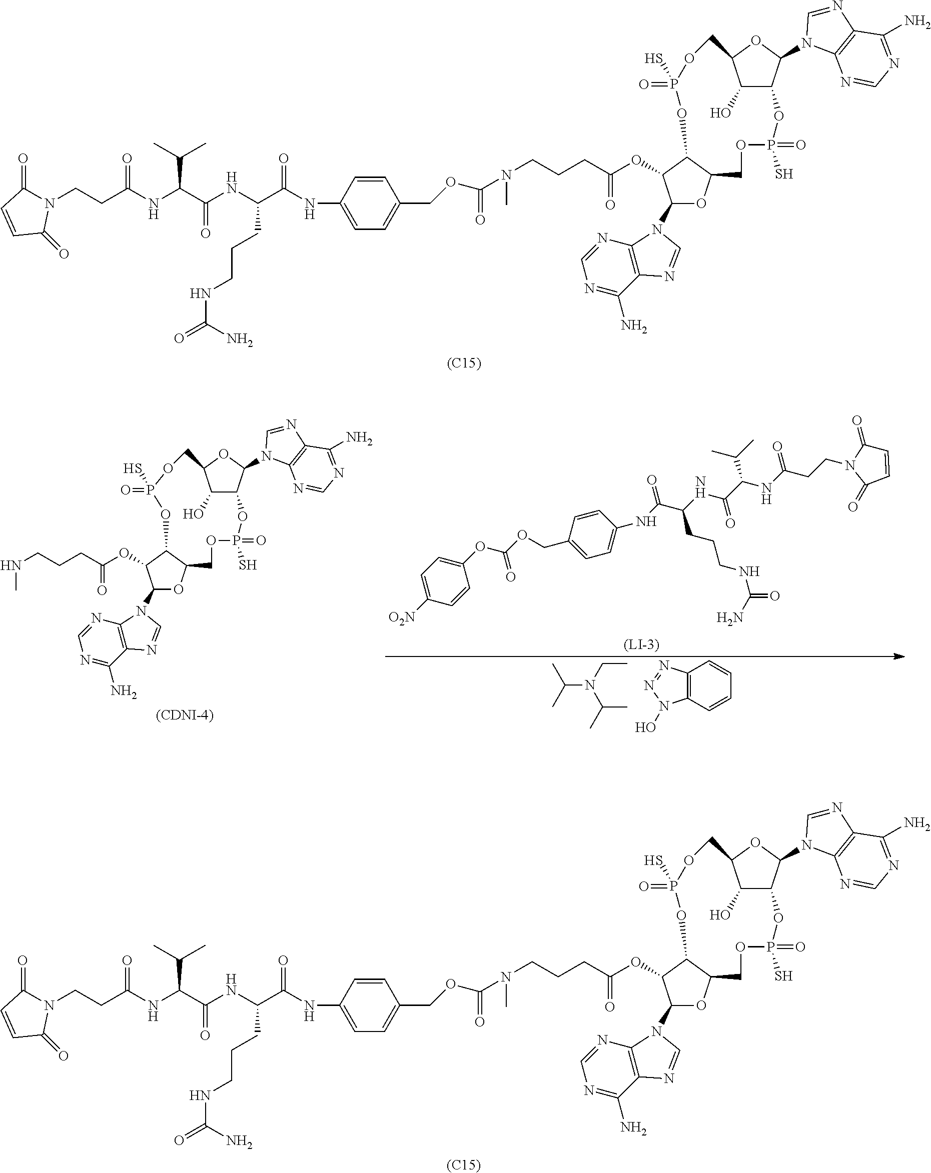

CDN intermediate (CDNI-4) (13.5 mg, 0.015 mmol) TFA salt in DMF was added to a solution of linker intermediate (LI-3) (10.5 mg, 0.015 mmol, 1.0 equiv), followed by the addition of DIEA (7.75 mg, 0.060 mmol) and HOAT (2.45 mg, 0.018 mmol). The mixture was stirred at room temperature for 16 hrs and then concentrated. The residue was dissolved in DMSO and purified by ISCO by using 15.5 gram, C18 aq column, eluted with 5-40% ACN in water with 10 mM TFA-HOAc. Fractions containing desired product were concentrated to obtain Compound (C15) (12.2 mg, 50% yield) as TEA salt. M+1=1346.20, tr=0.732 min.

### Example 3-5: Synthesis of Compound 16 (C16)

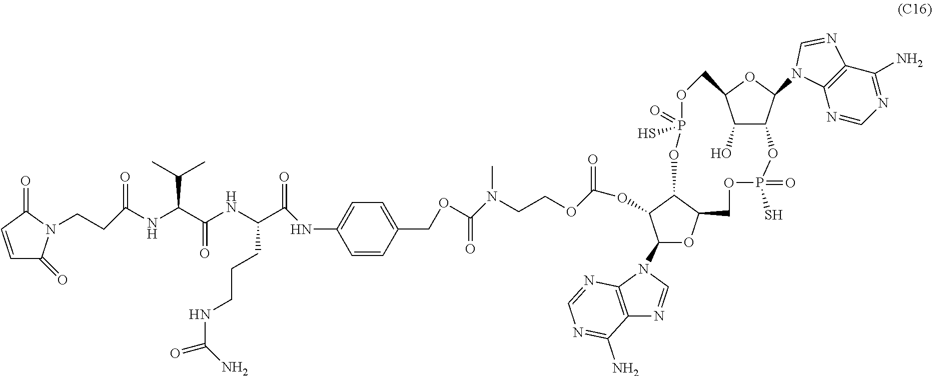 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(N)ncnc65)[C@H](O[P@](=O)(S)OC[C@H]3O[C@H]2n2cnc3c(N)ncnc32)[C@@H]4O)cc1]

Compound (C16) was synthesized using the methods describe for the synthesis of Compound (C15), except CDN intermediate (CDNI-5) TFA salt was used in place of CDN intermediate (CDNI-4).

Compound (C16) (7.6 mg, 31.3% yield) as TFA salt. LCMS M/2+1=681.8, tr=1.025 min.

### Example 3-6: Synthesis of Compound 17 (C17)

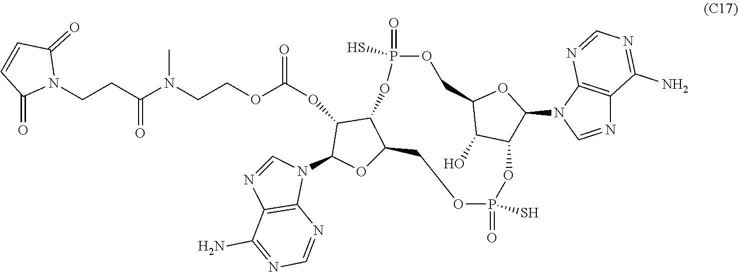 [SMILES: CN(CCOC(=O)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(N)ncnc21)[C@@H]3O)C(=O)CCN1C(=O)C=CC1=O]

TEA (6.7 mg, 0.066 mmol) and HATU (5.0 mg, 0.013 mmol) was added to a solution of 3-(2,5-dioxo-2,5-dihydro-1H-pyrrol-1-yl)propanoic acid (2.2 mg, 0.013 mmol)) in DMF (1 mL) and the mixture was stirred for 5 mins. CDN intermediate (CDNI-3) (15 mg, 0.013 mmol) in DMF (1 ml) was then added and the mixture was stirred for 18 hrs at room temperature and then concentrated. The residue was dissolved in DMSO (2 ml) and then purified by mass triggered reverse phase HPLC, using C18 column, eluted with 5-25% acetonitrile-H2O containing 0.05% TFA. Fractions containing desired product were lyophilized to obtain Compound (C17) (14.3 mg, 88% yield) as TFA salt. LCMS M+1=943.1 tr=0.561 min.

### Example 3-7: Synthesis of Compound 18 (C18)

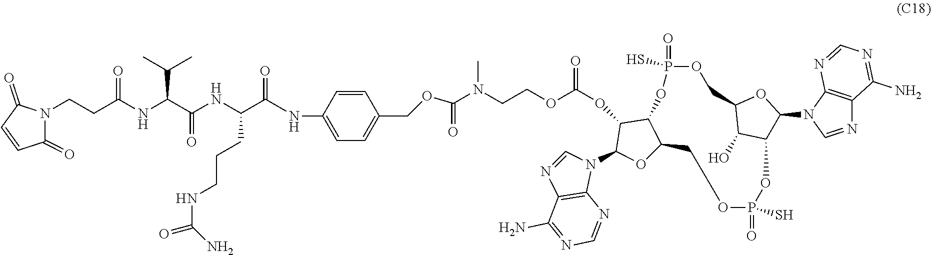 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(N)ncnc65)[C@H](O[P@](=O)(S)OC[C@H]3O[C@H]2n2cnc3c(N)ncnc32)[C@@H]4O)cc1]

CDN intermediate (CDNI-3) (20 mg, 0.018 mmol), DIEA (23 mg, 0.18 mmol) and HOAT (2.4 mg, 0.018 mmol) were added to a solution of linker intermediate (LI-3) (13.5 mg, 0.019 mmol) in DMF (1 mL) and the mixture was stirred for 18 hours at room temperature and then concentrated. The residue was dissolved in DMSO (2 ml) and then was pre-purified by ISCO using 15.5 g C18 column, eluted with 5-35% ACN-water containing 0.05% TFA. Fractions containing the desired product were combined and then purified by mass triggered reverse phase HPLC, C18 column, eluted with 10-30% acetonitrile-H₂O containing 0.05% TFA. Fractions containing the desired product were combined, and lyophilized to obtain Compound (C18) (12.3 mg, 39.8% yield) as TFA salt. LCMS M+1=1348.2, M/2+1=674.8, tr=0.842 min.

### Example 3-8: Synthesis of Compound 1 (C1)

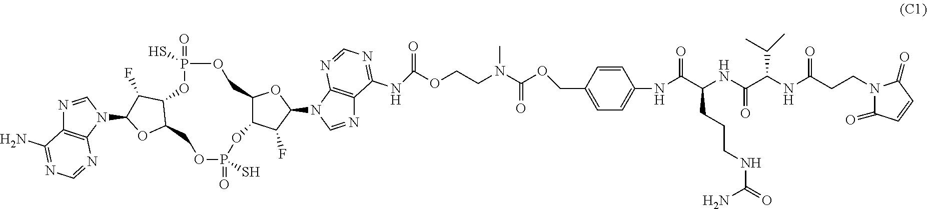 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Linker intermediate (LI-3) (36.7 mg, 0.053 mmol) was added to a solution of CDN intermediate (CDNI-1) (60 mg, 0.053 mmol) in DMF (5 ml), followed by the addition of DIEA (68.2 mg, 0.527 mmol) and HOAT (7.2 mg, 0.053 mmol). The mixture was stirred at room temperature for 16 hrs and then concentrated. The residue was dissolved in DMSO and pre-purified by ISCO by using 15.5 g C18 aq column, eluted with 5-35% ACN in water with 0.05% TFA. After purification, fractions were concentrated and then purified by mass triggered reverse phase HPLC, C18 column, eluted with 5-33% acetonitrile-H2O containing 0.05% TFA. Fractions containing desired product were concentrated to obtain Compound (C1) (55.4 mg, 68.1% yield) as TFA salt. LCMS M/2+1=676.8, M+1=1352.3, tr=0.753 min. ¹H NMR (500 MHz, DMSO-d₆) δ 10.01 (s, 1H), 9.42 (b, 1H), 8.56 (d, J=15.2 Hz, 1H), 8.31 (s, 1H), 8.16 (dd, J=13.1, 7.4 Hz, 1H), 8.04 (d, J=8.4 Hz, 1H), 7.62 (d, J=8.1 Hz, 1H), 7.48 (d, J=8.0 Hz, 1H), 7.32 (d, J=8.1 Hz, 1H), 7.18 (s, 1H), 7.02 (s, 2H), 6.43 (d, J=16.6 Hz, 2H), 6.18 (s, 2H), 5.61 (s, 1H), 5.50 (s, 1H), 5.13 (m, 3H), 5.02 (s, 1H), 4.93 (s, 1H), 4.55-4.34 (m, 6H), 4.27 (t, J=5.3 Hz, 2H), 4.19 (dd, J=8.5, 6.7 Hz, 1H), 3.87 (d, J=12.1 Hz, 2H), 3.63 (q, J=7.0, 6.6 Hz, 2H), 3.54 (s, 2H), 3.19-2.88 (m, 5H), 2.48 (q, J=7.4 Hz, 1H), 2.07-1.94 (m, 1H), 1.75 (m, 1H), 1.65 (m, 1H), 1.46 (m, 3H), 0.87 (dd, J=13.9, 6.8 Hz, 6H).

### Example 3-9: Synthesis of Compound 2 (C2)

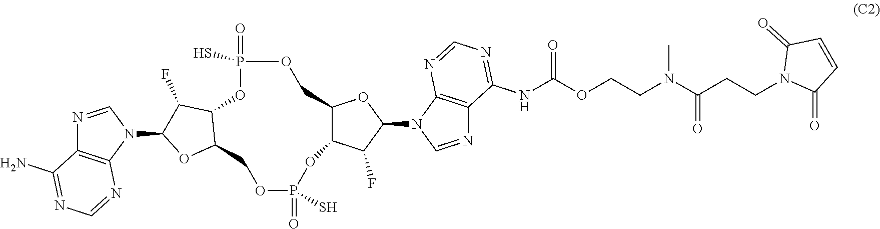 [SMILES: CN(CCOC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F)C(=O)CCN1C(=O)C=CC1=O]

TEA (1.3 mg, 0.013 mmol) and HATU (5 mg, 0.013 mmol) were added to a solution of 3-(2,5-dioxo-2,5-dihydro-1H-pyrrol-1-yl)propanoic acid (2.2 mg, 0.013 mmol) in DMF (1 mL) and the mixture was stirred for 5 mins. A solution of CDN intermediate (CDNI-1) TFA salt (15 mg, 0.013 mmol) in DMF (1 ml) was then added and the mixture was stirred for 18 hrs at room temperature and then concentrated. The residue was dissolved in DMSO (2 ml) and then purified by mass triggered reverse phase HPLC using C18 column, eluted with 5-25% acetonitrile-H₂O containing 0.05% TFA. Fractions containing desired product were lyophilized to obtain Compound (C2) (8.7 mg, 59% yield) as TFA salt. LCMS M+1=947.1, tr=0.646 min.

### Example 3-10: Synthesis of Compound 3 (C3)

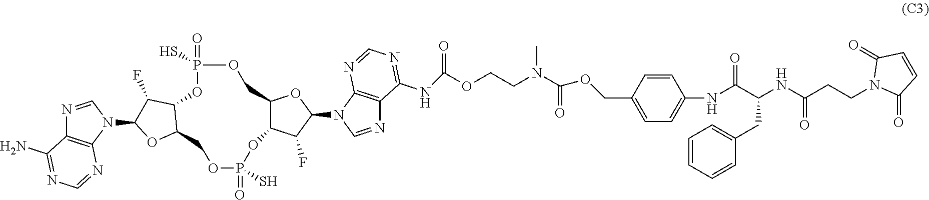 [SMILES: CN(CCOC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F)C(=O)OCc1ccc(NC(=O)[C@@H](Cc2ccccc2)NC(=O)CCN2C(=O)C=CC2=O)cc1]

Compound (C3) was synthesized using the methods describe for the synthesis of Compound (C2), except linker intermediate (LI-4) was used in place of 3-(2,5-dioxo-2,5-dihydro-1H-pyrrol-1-yl)propanoic acid.

Compound (C3) (4.5 mg, 26% yield) as TFA salt. LCMS M+1=1243.3, tr=0.924 min.

### Example 3-11: Synthesis of Compound 4 (C4)

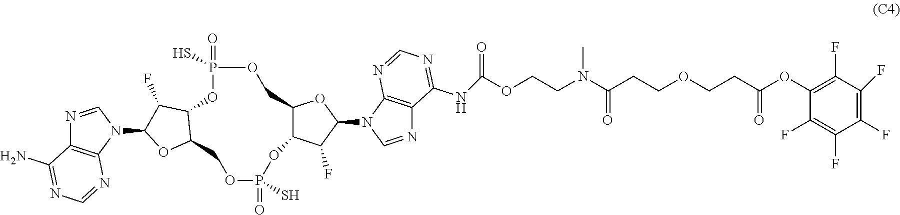 [SMILES: CN(CCOC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F)C(=O)CCOCCC(=O)Oc1c(F)c(F)c(F)c(F)c1F]

Compound (C4) was synthesized using the methods describe for the synthesis of Compound (C2), except bis(perfluorophenyl) 3,3′-oxydipropionate (purchased from Broadpharm, San Diego) was used in place of 3-(2,5-dioxo-2,5-dihydro-1H-pyrrol-1-yl)propanoic acid. Compound (C4) (10.5 mg, 46.5% yield) as TFA salt. LCMS M+1=1106.0, tr=0.930 min.

### Example 3-12: Synthesis of Compound 5 (C5)

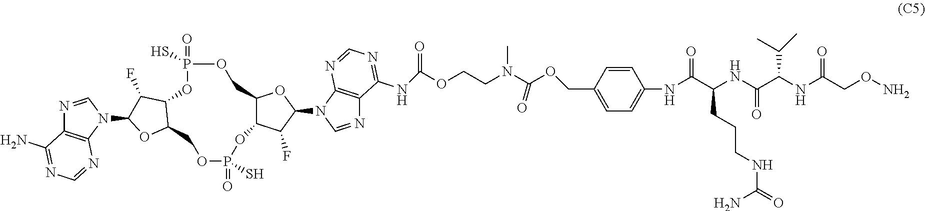 [SMILES: CC(C)[C@H](NC(=O)CON)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Step 1: DIEA (0.033 mL, 0.186 mmol) was added to a solution of CDN intermediate (CDNI-2) (26.6 mg, 0.019 mmol) and 2,5-dioxopyrrolidin-1-yl 2-(((((9H-fluoren-9-yl)methoxy)carbonyl)amino)oxy)acetate (15.28 mg, 0.037 mmol) in DMF (1 ml). The mixture was stirred at room temperature for 1 h and then concentrated. The residue was purified by reverse phase ISCO C18 50 g column, eluted with 10-50% acetonitrile-H₂O aqueous containing 10 mM HOAc Et₃N. Fractions containing desired product were concentrated to obtain 4-((95,125)-1-(9H-fluoren-9-yl)-9-isopropyl-3,7,10-trioxo-12-(3-ureidopropyl)-2,5-dioxa-4,8,11-triazatridecan-13-amido)benzyl (2-(((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,Z-j][1,3,7,9]tetraoxa[2,8]diphosphacyclododecin-2-yl)-9H-purin-6-yl)carbamoyl)oxy)ethyl)(methyl)carbamate (6 mg, 25% yield) as Et₃N salt. LCMS M/2+1=748.8, tr=0.966 min.

Step 2: 44(95,125)-1-(9H-fluoren-9-yl)-9-isopropyl-3,7,10-trioxo-12-(3-ureidopropyl)-2,5-dioxa-4,8,11-triazatridecan-13-amido)benzyl (2-(((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamoyl)oxy)ethyl)(methyl)carbamate (6.0 mg, 0.0035 mmol) triethylammonium salt was dissolved in ACN (2 ml) and water (2 ml) and LiOH (20 mg) was added. The mixture was stirred at room temperature for 4 hrs, neutralized with HOAc (0.06 ml) and then concentrated. The residue was purified by reverse phase ISCO 15.5 g C18 aq column, eluted with 5-40% acetonitrile-H₂O containing 0.05% TFA. Fractions containing desired product were concentrated to obtain Compound (C5) (2.8 mg, 36.9% yield) as TFA salt. LCMS M/2+1=637.8 tr=0.676 min.

### Example 3-13: Synthesis of Compound 6 (C6)

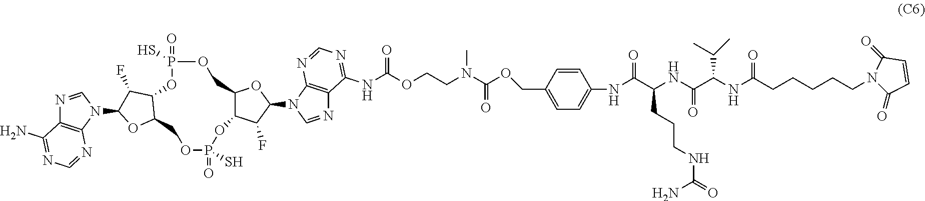 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C6) was synthesized using the methods describe for the synthesis of Compound (C14), except CDN intermediate (CDNI-1) was used in place of CDN intermediate (CDNI-3). Compound (C6) (1.2 mg, 24% yield) as TFA salt. LCMS M/2+1=697.8, M+1=1394.5, tr=0.782 min.

### Example 3-14: Synthesis of Compound 7 (C7)

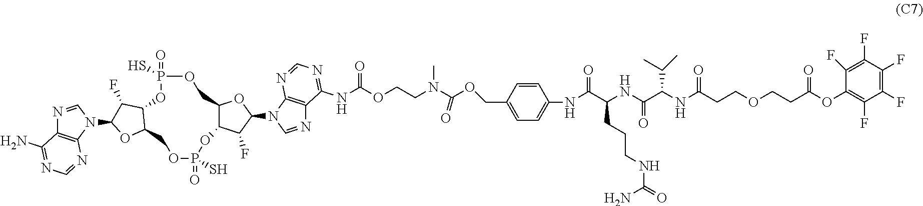 [SMILES: CC(C)[C@H](NC(=O)CCOCCC(=O)Oc1c(F)c(F)c(F)c(F)c1F)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C7) was synthesized using the methods describe for the synthesis of Compound (C4), except CDN intermediate (CDNI-2) was used in place of CDN intermediate (CDNI-1). Compound (C7) (5.3 mg, 55.3% yield) as TFA salt. LCMS M/2+1=756.3, tr=0.975 min.

### Example 3-15: Synthesis of Compound 8 (C8)

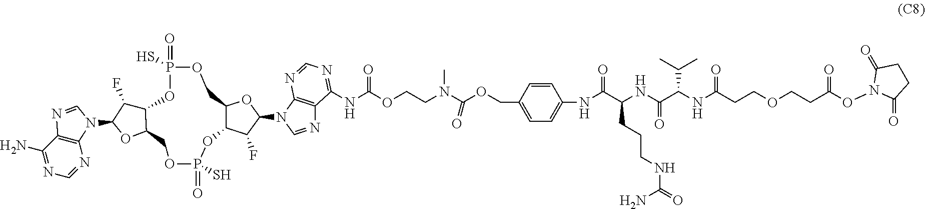 [SMILES: CC(C)[C@H](NC(=O)CCOCCC(=O)ON1C(=O)CCC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

DIEA (0.01 ml, 0.056 mmol) was added to a solution of CDN intermediate (CDNI-2) (8 mg, 0.0056 mmol) and bis(2,5-dioxopyrrolidin-1-yl) 3,3′-oxydipropionate (5.98 mg, 0.017 mmol) ((Bis-PEG1-NHS ester purchased from Broadpharm, San Diego) in DMF (1 ml). The mixture was stirred at room temperature for 2 hours and then concentrated. The residue was purified by mass triggered reverse phase HPLC, using C18 column, eluted with 10-33% acetonitrile-H₂O containing 0.05% TFA. Fractions containing desired product were lyophilized to obtain Compound (C8) (5.7 mg, 62.2% yield) as TFA salt. LCMS M/2+1=721.8, tr=0.755 min.

### Example 3-16: Synthesis of Compound 9 (C9)

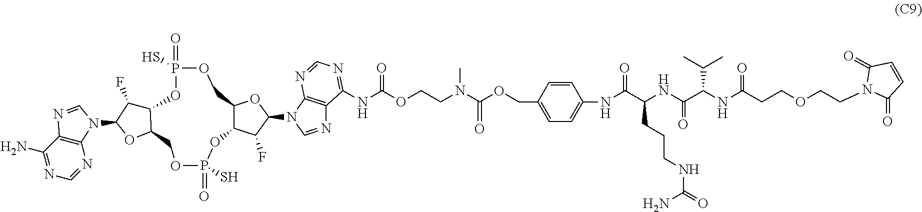 [SMILES: CC(C)[C@H](NC(=O)CCOCCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C9) was synthesized using the methods describe for the synthesis of Compound (C1), except linker intermediate (LI-5) was used in place of linker intermediate (LI-3). Compound (C9) (6.8 mg, 52.6% yield) as TFA salt. LCMS M/2+1=698.8, tr=0.758 min.

### Example 3-17: Synthesis of Compound 10 (C10)

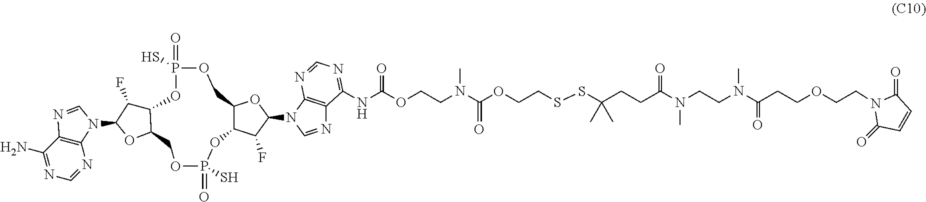 [SMILES: CN(CCN(C)C(=O)CCC(C)(C)SSCCOC(=O)N(C)CCOC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F)C(=O)CCOCCN1C(=O)C=CC1=O]

Compound (C10) was synthesized using the methods describe for the synthesis of Compound (C1), except linker intermediate (LI-2) was used in place of linker intermediate (LI-3).

Compound (C10) (7.3 mg, 55.3% yield) as TFA salt. LCMS M+1=1311.2, M/2+1=656.2, tr=0.845 min.

### Example 3-18: Synthesis of Compound 11 (C11)

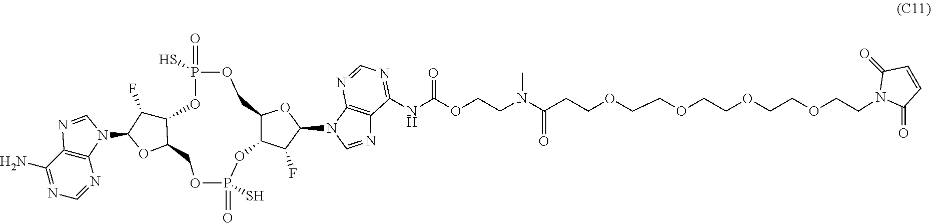 [SMILES: CN(CCOC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F)C(=O)CCOCCOCCOCCOCCN1C(=O)C=CC1=O]

Compound (C11) was synthesized using the methods describe for the synthesis of Compound (C1), except 1-(2,5-dioxo-2,5-dihydro-1H-pyrrol-1-yl)-3,6,9,12-tetraoxapentadecan-15-oic acid (MPEG4-acid purchased from Broadpharm, San Diego) was used in place of linker intermediate (LI-3).

Compound (C11) 10.9 mg (37.6% yield) LCMS M+1=1123.1, tr=0.722 min.

### Example 3-19: Synthesis of Compound 19 (C19)

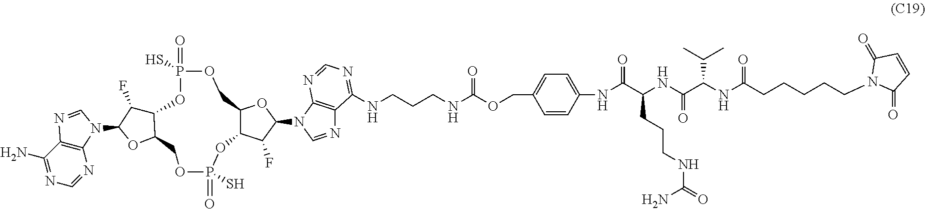 [SMILES: CC(C)[C@H](NC(=O)CCCCCN1C(=O)C=CC1=O)C(=O)N[C@@H](CCCNC(N)=O)C(=O)Nc1ccc(COC(=O)NCCCNc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C19) was synthesized using the methods describe for the synthesis of Compound (C2), except CDN intermediate (CDNI-10) was used in place of CDN intermediate (CDNI-1) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (MC-vc-pab-PNP purchased from Levena Biopharma, San Diego) was used in place of 3-(2,5-dioxo-2,5-dihydro-1H-pyrrol-1-yl)propanoic acid.

Compound (C19) (1.1 mg, 20% yield) as TFA salt. LCMS M/2+1=675.8 tr=0.776 min.

### Example 3-20: Synthesis of Compound 20 (C20)

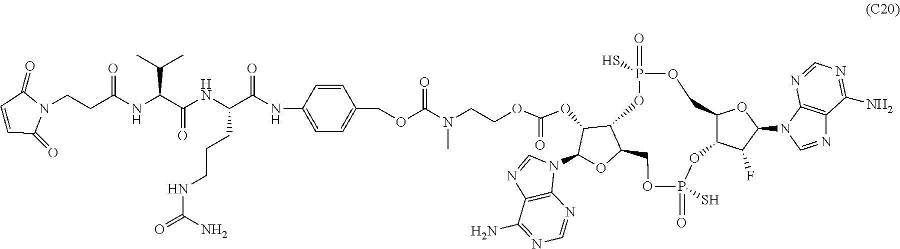 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(N)ncnc65)[C@H](F)[C@@H]4O[P@](=O)(S)OC[C@H]3O[C@H]2n2cnc3c(N)ncnc32)cc1]

Compound (C20) was synthesized using the methods describe for the synthesis of Compound (C1), except CDN intermediate (CDNI-6) was used in place of CDN intermediate (CDNI-1). Compound (C20) (4.2 mg, 30% yield) as TFA salt. LCMS M/2+1=675.8, M+1=1350.3, tr=0.751 min. 1H NMR (500 MHz, DMSO-d6) δ 9.99 (s, 1H), 9.28 (s, 2H), 8.98 (s, 3H), 8.14 (d, J=7.4 Hz, 2H), 8.04 (d, J=8.3 Hz, 2H), 7.99 (s, 1H), 7.64 (d, J=8.2 Hz, 2H), 7.36 (d, J=8.1 Hz, 2H), 7.03 (s, 2H), 6.49 (d, J=46.4 Hz, 2H), 6.03 (s, 1H), 5.70 (d, J=49.8 Hz, 2H), 5.21-4.83 (m, 5H), 4.68-4.32 (m, 9H), 4.28-4.13 (m, 2H), 3.13 (qd, J=7.3, 4.9 Hz, 2H), 3.02 (d, J=11.7 Hz, 6H), 1.97 (dt, J=12.7, 6.2 Hz, 1H), 1.86-1.55 (m, 2H), 1.45 (d, J=32.2 Hz, 2H), 1.31-1.11 (m, 4H), 0.86 (dd, J=16.0, 6.7 Hz, 8H).

### Example 3-21: Synthesis of Compound 21 (C21)

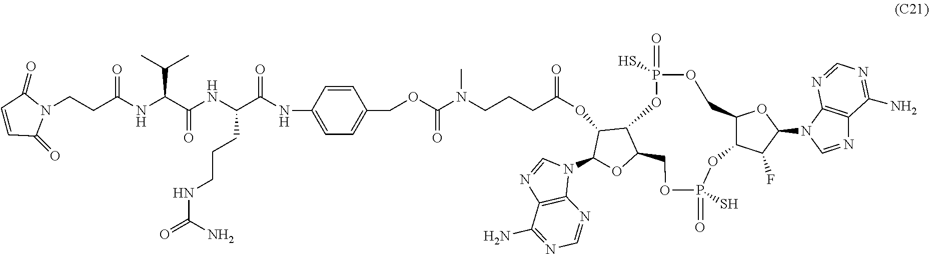 [SMILES: CC(C)[C@H](NC(=O)CCN1C(=O)C=CC1=O)C(=O)N[C@@H](CCCNC(N)=O)C(=O)Nc1ccc(COC(=O)N(C)CCCC(=O)O[C@@H]2[C@@H]3O[P@](=O)(S)OC[C@H]4O[C@@H](n5cnc6c(N)ncnc65)[C@H](F)[C@@H]4O[P@](=O)(S)OC[C@H]3O[C@H]2n2cnc3c(N)ncnc32)cc1]

Compound (C21) was synthesized using the methods describe for the synthesis of Compound (C1), except CDN intermediate (CDNI-7) was used in place of CDN intermediate (CDNI-1). Compound (C21) (12.2 mg, 50% yield) as TEA salt. M+1=1348.20, tr=0.721 min.

### Example 3-22: Synthesis of Compound 22 (C22)

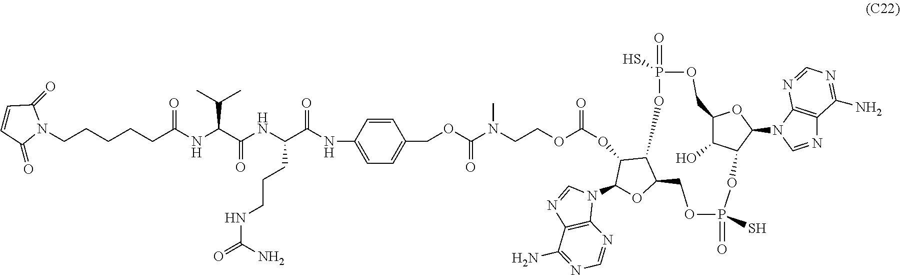 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(N)ncnc65)[C@H](O[P@@](=O)(S)OC[C@H]3O[C@H]2n2cnc3c(N)ncnc32)[C@@H]4O)cc1]

Compound (C22) was synthesized using the methods describe for the synthesis of Compound (C19), except CDN intermediate (CDNI-8) was used in place of CDN intermediate (CDNI-10). Compound (C22) (0.9 mg, 34.1% yield) as TFA salt. LCMS M/2+1=695.8, M+1=1391, tr=0.695 min.

### Example 3-23

### Synthesis of Compound 23a (C23a)

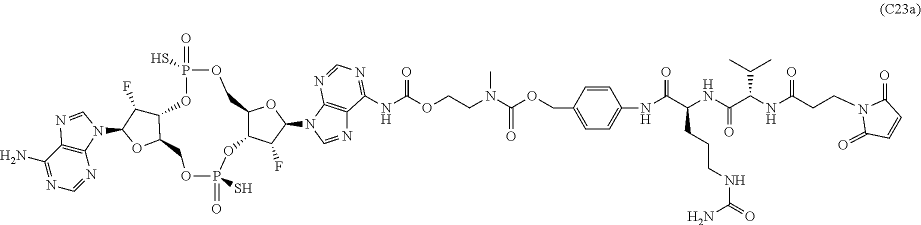 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@](=O)(S)O[C@H]3[C@H]2F)cc1]

Compound (C23a) was synthesized using the methods describe for the synthesis of Compound (C1), except CDN intermediate (CDNI-9) was used in place of CDN intermediate (CDNI-1). Compound (C23a) (12.7 mg, 51.7% yield) as TFA salt. LCMS M/2+1=676.7, tr=0.700 min.

### b) Synthesis of Compound 23b (C23b)

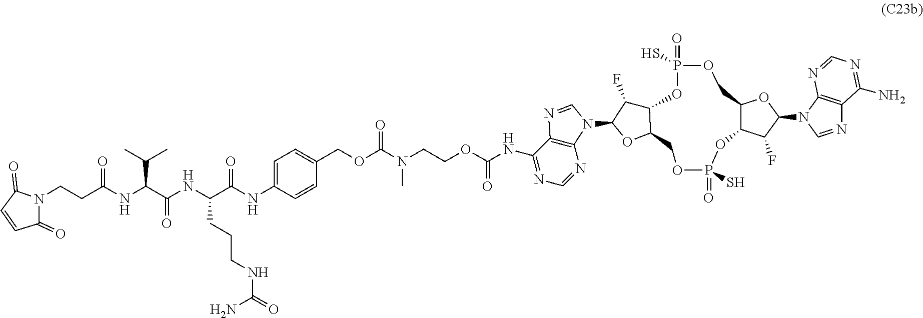 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@](=O)(S)O[C@H]4[C@@H](F)[C@H](n5cnc6c(N)ncnc65)O[C@@H]4CO[P@@](=O)(S)O[C@H]3[C@H]2F)cc1]

Compound (23b) was obtained during the synthesis of Compound (23a). Compound (C23a) and Compound (23b) were not separated. (12.7 mg, 51.7% yield) as TFA salt. LCMS M/2+1=676.7, tr=0.700 min.

### Example 3-24: Synthesis of Compound 24 (C24)

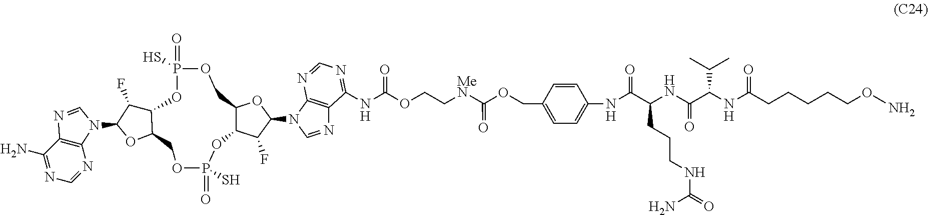 [SMILES: CC(C)[C@H](NC(=O)CCCCCON)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

HATU (1.9 mg, 0.005 mmol) was added to a mixture of (Z)-6-(((1-ethoxyethylidene)amino)oxy)hexanoic acid (1.2 mg, 0.0056 mmol) and DIEA (2.2 mg, 0.017 mmol) in DMF (1 ml). The mixture was then stirred at room temperature for 5 min and then added to a solution of CDN intermediate (CDNI-2) (4 mg, 0.0028 mmol) in DMF (1 ml). The mixture was then stirred for 5 hours at room temperature for 16 hours and then concentrated to give the protected derivative ethyl (Z)-N-((6-(((S)-1-(((S)-1-((4-((((2-(((9-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)-9H-purin-6-yl)carbamoyl)oxy)ethyl)(methyhcarbamoyl)oxy)methyl)phenyl)amino)-1-oxo-5-ureidopentan-2-yl)amino)-3-methyl-1-oxobutan-2-yl)amino)-6-oxohexyl)oxy)acetimidate. LCMS M/2+1=700.8, tr=0.890 min.

Purification of the residue by reverse phase HPLC, ISCO C18 50 g column, eluted with 10-50% acetonitrile-H₂O containing 0.05% TFA resulted in loss of the protecting group. Fractions containing desired product Compound (C-24) were concentrated further purified by reverse phase ISCO C18 column, eluted with 5-40% acetonitrile-H₂O containing 0.05% TFA to obtain Compound (C-24) (2.2 mg, 47.9% yield) as TFA salt. LCMS M/2+1=665.8, tr=0.697 min.

Note: Z)-6-(((1-ethoxyethylidene)amino)oxy)hexanoic acid was prepared from ethyl-(N-hedroxyacetimidate and 6-bromohexanoic acid in the presence of LiOH using the method described in Biomacromolecules 6(5) 2648, 2005.

### Example 3-25

### a) Synthesis of Compound 25a (C25a)

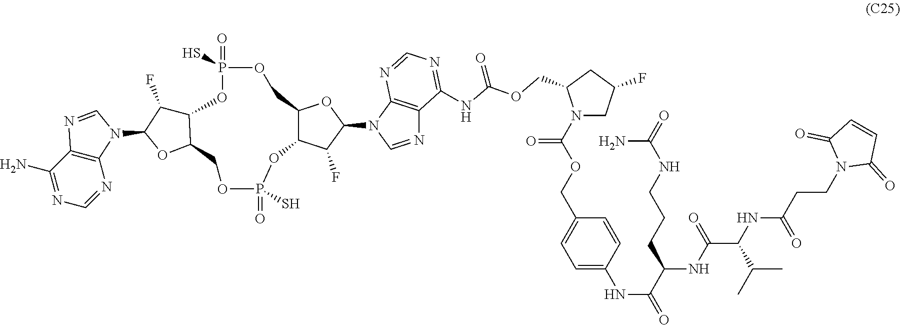 [SMILES: CC(C)[C@@H](NC(=O)CCN1C(=O)C=CC1=O)C(=O)N[C@H](CCCNC(N)=O)C(=O)Nc1ccc(COC(=O)N2C[C@@H](F)C[C@H]2COC(=O)Nc2ncnc3c2ncn3[C@@H]2O[C@@H]3CO[P@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C25a) was synthesized using the methods describe for the synthesis of Compound (C1), except CDN intermediate (CDNI-11) was used in place of CDN intermediate (CDNI-1). Compound (C25a) (7.5 mg, 37.1% yield) as TFA salt. LCMS M/2+1=698.8, tr=0.715 min.

### b) Synthesis of Compound 25b (C25b)

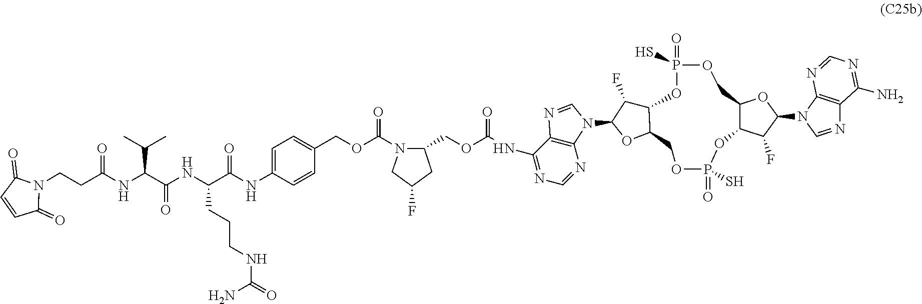 [SMILES: CC(C)[C@H](NC(=O)CCN1C(=O)C=CC1=O)C(=O)N[C@@H](CCCNC(N)=O)C(=O)Nc1ccc(COC(=O)N2C[C@@H](F)C[C@H]2COC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@](=O)(S)O[C@H]3[C@H]2F)cc1]

Compound (25b) was obtained during the synthesis of Compound (25a). Compound (C23a) and Compound (25b) were not separated. (7.5 mg, 37.1% yield) as TFA salt. LCMS M/2+1=698.8, tr=0.715 min

### Example 3-26: Synthesis of Compound 26 (C26)

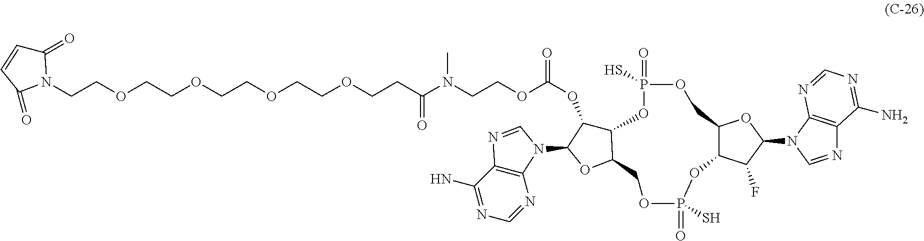 [SMILES: CN(CCOC(=O)O[C@@H]1[C@@H]2O[P@](=O)(S)OC[C@H]3O[C@@H](n4cnc5c(N)ncnc54)[C@H](F)[C@@H]3O[P@](=O)(S)OC[C@H]2O[C@H]1n1cnc2c(N)ncnc21)C(=O)CCOCCOCCOCCOCCN1C(=O)C=CC1=O]

DIEA (0.019 mL, 0.110 mmol) and HATU (9.2 mg, 0.024 mmol) were added to a solution of 1-(2,5-dioxo-2,5-dihydro-1H-pyrrol-1-yl)-3,6,9,12-tetraoxapentadecan-15-oic acid (Mal-PEG4-acid) (8.4 mg, 0.024 mmol) in DMF (1 ml) and the mixture was stirred for 5 mins and then added a solution of CDN intermediate (CDNI-7) (25 mg, 0.022 mmol) in DMF (1 ml). The reaction was then stirred at room temperature for 16 hrs and then concentrated. The residue was purified by reverse phase ISCO C18 column, eluted with 5-40% acetonitrile-H₂O with the aqueous phase containing 10 mM Et₃N HOAc. Fractions containing desired product were lyophilized to obtain Compound (C-26) (23.2 mg, 76% yield) as TEA salt. LCMS M+1=1121.1 tr=0.733 min. 1H NMR (500 MHz, DMSO-d₆) δ 8.66 (d, J=3.7 Hz, 2H), 7.96-7.75 (m, 2H), 7.06 (s, 2H), 6.32 (d, J=14.0 Hz, 1H), 6.26 (d, J=3.1 Hz, 1H), 5.81 (t, J=5.8 Hz, 1H), 5.63 (d, J=52.4 Hz, 1H), 5.24-5.00 (m, 2H), 4.58-4.26 (m, 6H), 3.89-3.72 (m, 3H), 3.72-3.63 (m, 2H), 3.64-3.54 (m, 3H), 3.54-3.47 (m, 12H), 3.16 (s, 2H), 3.01 (q, J=7.2 Hz, 15H), 2.95 (s, 1H), 2.74-2.61 (m, 2H), 1.94 (s, 1H), 1.13 (t, J=7.2 Hz, 21H).

### Example 3-27: Synthesis of Compound 27 (C27)

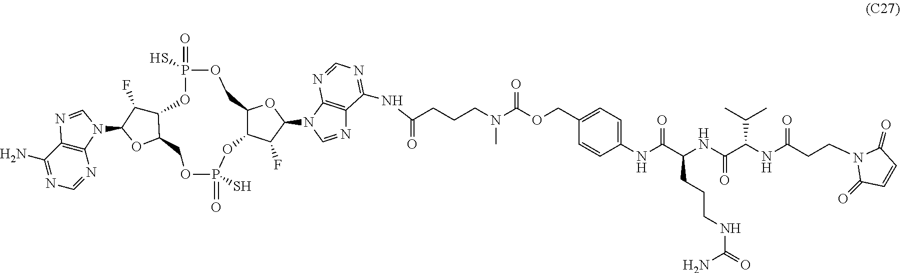 [SMILES: CC(C)[C@H](NC(=O)CCN1C(=O)C=CC1=O)C(=O)N[C@@H](CCCNC(N)=O)C(=O)Nc1ccc(COC(=O)N(C)CCCC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C27) was synthesized using similar methods describe for the synthesis of Compound (C15), except CDN intermediate (CDNI-12) was used in place of CDN intermediate (CDNI-4) and the C18 column was eluted with 5-50% acetonitrile-H₂O (aqueous phase containing 10 mM Et₃N HOAc). Fractions containing the desired product were concentrated to obtain Compound (C27) as Et₃N salt (1 mg, 11% yield). LCMS M/2+1=675.8, tr=0.758 min.

### Example 3-28: Synthesis of Compound 28 (C28)

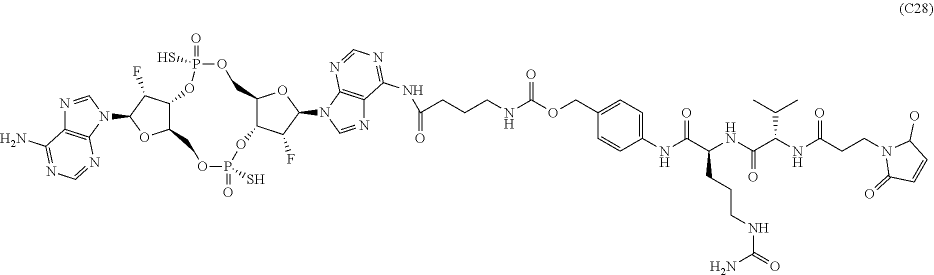 [SMILES: CC(C)[C@H](NC(=O)CCN1C(=O)C=CC1O)C(=O)N[C@@H](CCCNC(N)=O)C(=O)Nc1ccc(COC(=O)NCCCC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C28) was synthesized using similar methods describe for the synthesis of Compound (C15), except CDN intermediate (CDNI-13) was used in place of CDN intermediate (CDNI-4). Compound (C28) (5.8 mg, 30% yield). LCMS M/2+1=668.8, tr=0.724 min.

### Example 3-29: Synthesis of Compound 29 (C29)

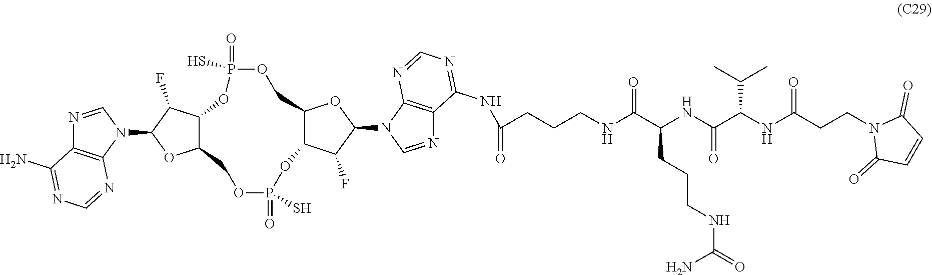 [SMILES: CC(C)[C@H](NC(=O)CCN1C(=O)C=CC1=O)C(=O)N[C@@H](CCCNC(N)=O)C(=O)NCCCC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F]

2,5-dioxopyrrolidin-1-yl 3-(2,5-dioxo-2,5-dihydro-1H-pyrrol-1-yl)propanoate (purchased from Combi-Blocks)(0.5 mg, 0.002 mmol) and DIEA (1.7 mg, 0.013 mmol) were added to a solution of intermediate CDNI-15 TFA salt (1.7 mg, 0.0013 mmol) in DMF (1 ml) and the reaction was stirred at rt for 72 hrs and then concentrated. The crude was purified by reverse phase ISCO using 15 g C18 aq column, eluted with 5-40% acetonitrile-H₂O (aqueous phase containing 10 mM Et₃N HOAc). Fractions containing desired product were concentrated to obtain Compound 29 (C29) as an Et₃N salt (2.3 mg, 111% yield). LCMS M+1=1187.1, tr=0.675 min.

### Example 3-30: Synthesis of Compound 30 (C30)

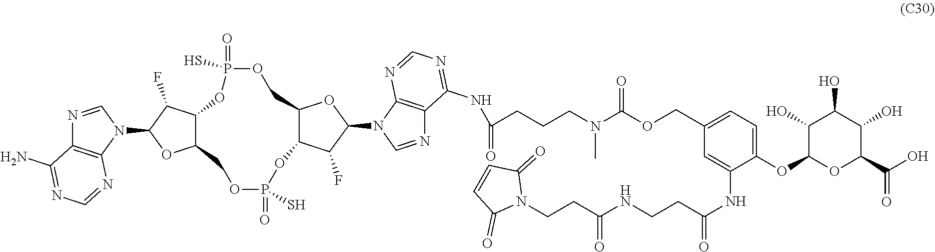 [SMILES: CN(CCCC(=O)Nc1ncnc2c1ncn2[C@@H]1O[C@@H]2CO[P@@](=O)(S)O[C@H]3[C@@H](F)[C@H](n4cnc5c(N)ncnc54)O[C@@H]3CO[P@@](=O)(S)O[C@H]2[C@H]1F)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1]

Compound (C30) was synthesized using similar methods describe for the synthesis of Compound (C29), except CDN intermediate (CDNI-16) was used in place of CDN intermediate (CDNI-15), the reaction mixture was stirred for 16 hrs and the crude was purified by reverse phase ISCO with 50 g C18 aq column and eluted with 5-35% acetonitrile-water (aqueous phase containing 10 mM Et₃N HOAc). Fractions with the desired product were combined and lyophilized to give Compound 30 (C30) as Et₃N salt (3.8 mg, 14% yield). LCMS M/2+1=680.2, tr=0.705 min.

### Example 3-31: Synthesis of Compound 31 (C31)

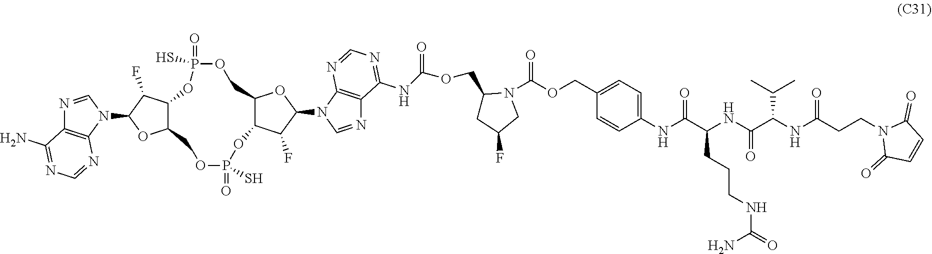 [SMILES: CC(C)[C@H](NC(=O)CCN1C(=O)C=CC1=O)C(=O)N[C@@H](CCCNC(N)=O)C(=O)Nc1ccc(COC(=O)N2C[C@@H](F)C[C@H]2COC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C31) was synthesized using the methods describe for the synthesis of Compound (C1), except CDN intermediate (CDNI-17) TFA salt was used in place of CDN intermediate (CDNI-1), the reaction was stirred at rt for 20 hours and purification was by ISCO using 15.5 C18 aq column, eluted with 5-40% acetonitrile-H₂O containing 10 mM Et₃N-HOAc. Fractions containing desired product were concentrated to obtain Compound 31 (C31) (4.3 mg, 76% yield) as TEA salt. LCMS M/2+1=698.8, tr=0.800 min.

### Example 3-32: Synthesis of Compound 32 (C32)

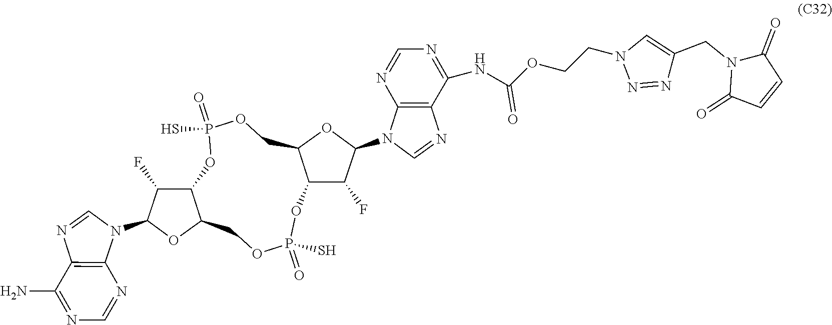 [SMILES: Nc1ncnc2c1ncn2[C@@H]1O[C@@H]2CO[P@@](=O)(S)O[C@H]3[C@@H](F)[C@H](n4cnc5c(NC(=O)OCCn6cc(CN7C(=O)C=CC7=O)nn6)ncnc54)O[C@@H]3CO[P@@](=O)(S)O[C@H]2[C@H]1F]

A solution of CDN intermediate (CDNI-18) Et₃N salt (20 mg, 0.022 mmol) and 1-(prop-2-yn-1-yl)-1H-pyrrole-2,5-dione (11.7 mg, 0.087 mmol) in 1:2 mixture of water-t-BuOH (4.5 ml) was degassed with N2, and a degassed solution of sodium L-ascobate (21.5 mg, 0.109 mmol) in water was added, followed by a degassed solution CuSO₄ (10.4 mg, 0.065 mmol) in water. The reaction mixture was stirred at rt for 1 hr and then lyophilized. The crude was purified by reverse phase ISCO using 50 g C18 column, eluted with 10-30% acetonitrile-H₂O (aqueous phase containing 10 mM Et₃N HOAc). Fractions containing the desired product were combined and lyophilized and repurify with reverse phase ISCO using 50 g C18 column, eluted with 10-30% acetonitrile-H₂O containing 0.05% TFA. Fractions containing desired product were lyophilized to obtain Compound 32 (C32) as TFA salt (1.9 mg, 6% yield). LCMS M+1=943.0, tr=0.725 min.

### Example 3-33: Synthesis of Compound 33 (C33)

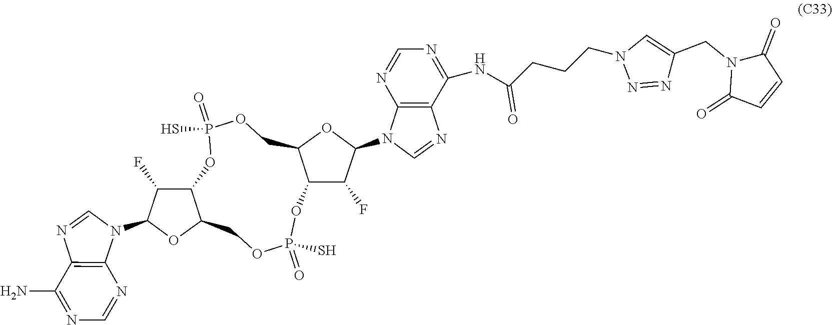 [SMILES: Nc1ncnc2c1ncn2[C@@H]1O[C@@H]2CO[P@@](=O)(S)O[C@H]3[C@@H](F)[C@H](n4cnc5c(NC(=O)CCCn6cc(CN7C(=O)C=CC7=O)nn6)ncnc54)O[C@@H]3CO[P@@](=O)(S)O[C@H]2[C@H]1F]

Compound (C33) was synthesized using the methods describe for the synthesis of Compound (C32), except CDN intermediate (CDNI-19) TFA salt was used in place of CDN intermediate (CDNI-18). Compound (C33) TFA salt (2.7 mg, 10% yield). LCMS M+1=941.0, tr=0.725 min.

### Example 3-34: Synthesis of Compound 34 (C34)

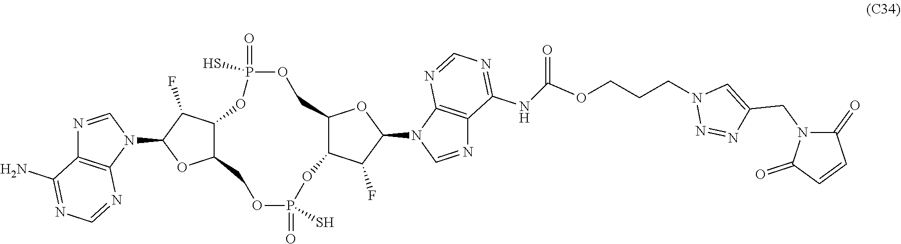 [SMILES: Nc1ncnc2c1ncn2[C@@H]1O[C@@H]2CO[P@@](=O)(S)O[C@H]3[C@@H](F)[C@H](n4cnc5c(NC(=O)OCCCn6cc(CN7C(=O)C=CC7=O)nn6)ncnc54)O[C@@H]3CO[P@@](=O)(S)O[C@H]2[C@H]1F]

Compound (C34) was synthesized using the methods describe for the synthesis of Compound (C32), except CDN intermediate (CDNI-20) TFA salt was used in place of CDN intermediate (CDNI-18). LCMS M+1=957.1, tr=0.693 min.

### Example 3-35: Synthesis of Compound 35 (C35)

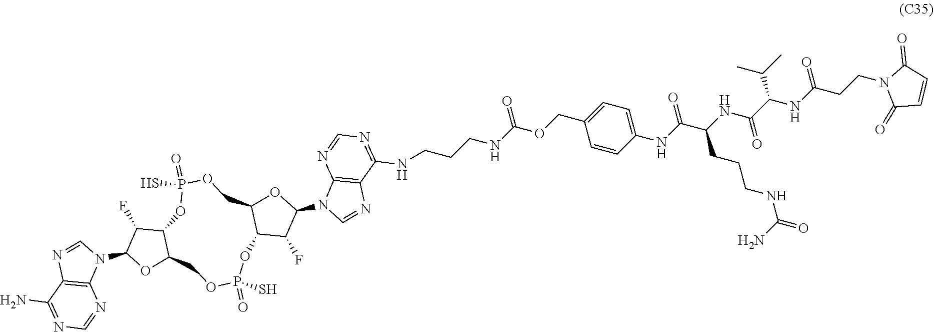 [SMILES: CC(C)[C@H](NC(=O)CCN1C(=O)C=CC1=O)C(=O)N[C@@H](CCCNC(N)=O)C(=O)Nc1ccc(COC(=O)NCCCNc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

Compound (C35) was synthesized using the methods describe for the synthesis of Compound (C1), except CDN intermediate (CDNI-10) TFA salt was used in place of CDN intermediate (CDNI-1), the reaction was stirred at rt for 1 day and purification was reverse phase ISCO using C18 column, eluted with 5-35% acetonitrile-H₂O (aqueous phase containing 10 mM Et₃N HOAc). Fractions containing desired product were concentrated to obtain Compound 35 (C35) as Et₃N salt (4.0 mg, 120% yield). LCMS M+1=1308.1, tr=0.761 min.

### Example 3-36: Synthesis of a Mixture of Compound 36a (C36a) and Compound 36b (C36b)

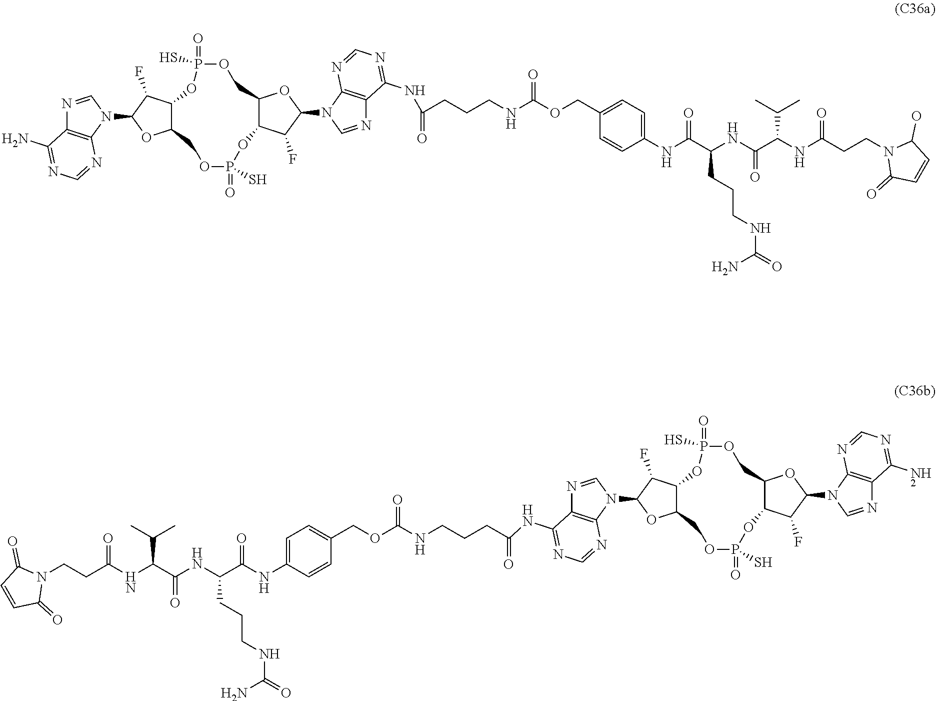 [SMILES: CC(C)[C@H](NC(=O)CCN1C(=O)C=CC1O)C(=O)N[C@@H](CCCNC(N)=O)C(=O)Nc1ccc(COC(=O)NCCCC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1 ; CC(C)[C@H](NC(=O)CCN1C(=O)C=CC1=O)C(=O)N[C@@H](CCCNC(N)=O)C(=O)Nc1ccc(COC(=O)NCCCC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)cc1]

The mixture of Compound 36a (C36a) and Compound 36b (C36b) was obtained using the methods describe for the synthesis of Compound (C1), except the mixture of CDN intermediates (CDNI-21a) and (CDNI-21b) TFA salt was used in place of CDN intermediate (CDNI-1), and an initial purification was by reverse phase ISCO using 15 g C18 column, eluted with 5-45% acetonitrile-H₂O (aqueous phase containing 10 mM Et₃N HOAc). Fractions containing desired product were concentrated and further purified by reverse phase ISCO by using 50 g C18 aq column, eluted with 5-35% acetonitrile-water with 0.05% TFA. Fractions containing desired product were concentrated and lyophilized to obtain to obtain the mixture of Compound 36a (C36a) and Compound 36b (C36b) as TFA salt (8.3 mg, 41% yield). LCMS M+1=1336.1, tr=0.799 min.

### Example 3-37: Synthesis of a Mixture of Compound 37a (C37a) and Compound 37b (C367b)

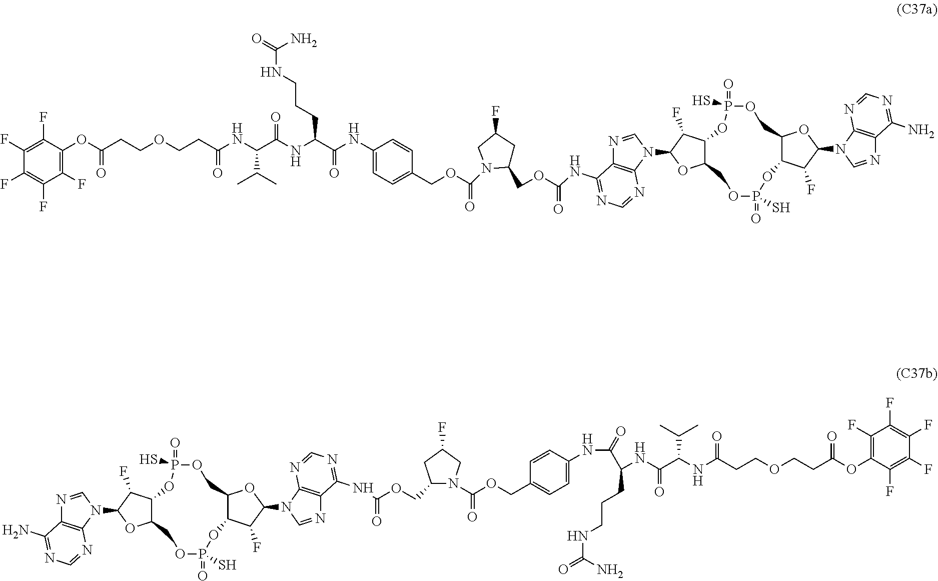 [SMILES: CC(C)[C@H](NC(=O)CCOCCC(=O)Oc1c(F)c(F)c(F)c(F)c1F)C(=O)N[C@@H](CCCNC(N)=O)C(=O)Nc1ccc(COC(=O)N2C[C@@H](F)C[C@H]2COC(=O)Nc2ncnc3c2ncn3[C@@H]2O[C@@H]3CO[P@@](=O)(S)O[C@H]4[C@@H](F)[C@H](n5cnc6c(N)ncnc65)O[C@@H]4CO[P@](=O)(S)O[C@H]3[C@H]2F)cc1 ; CC(C)[C@H](NC(=O)CCOCCC(=O)Oc1c(F)c(F)c(F)c(F)c1F)C(=O)N[C@@H](CCCNC(N)=O)C(=O)Nc1ccc(COC(=O)N2C[C@@H](F)C[C@H]2COC(=O)Nc2ncnc3c2ncn3[C@@H]2O[C@@H]3CO[P@](=O)(S)O[C@H]4[C@@H](F)[C@H](n5cnc6c(N)ncnc65)O[C@@H]4CO[P@@](=O)(S)O[C@H]3[C@H]2F)cc1]

DIEA (11.0 mg, 0.086 mmol) was added to a solution of CDN intermediate (CDNI-22a and CDI-22b) (12.6 mg, 0.0086 mmol) and bis(perfluorophenyl) 3,3′-oxydipropionate (Bis-PEG1-PFP ester purchased from Broadpharm) (12.7 mg, 0.026 mmol) in DMF (1 ml). The reaction was stirred at rt for 2 hours and then concentrated. The residue was purified by reverse phase ISCO by using 30 g C18 aq column, eluted with 5-100% acetonitrile-water with 0.05% TFA. Fractions containing desired product were concentrated and lyophilized to obtain mixture of Compound 37a and 37b (C37a and C37b) as TFA salt (6.2 mg, 38.6% yield). LCMS M/2+1=778.3, tr=0.974 min.

### Example 3-38: Synthesis of Compound 38 (C38)

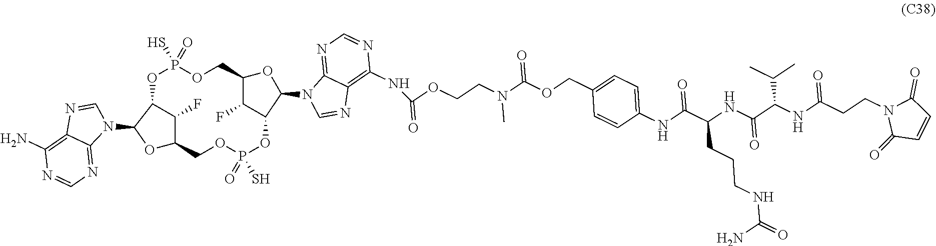 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@H]3CO[P@@](=O)(S)O[C@@H]4[C@H](F)[C@@H](CO[P@@](=O)(S)O[C@@H]2[C@@H]3F)O[C@H]4n2cnc3c(N)ncnc32)cc1]

Compound (C38) was synthesized using similar methods describe for the synthesis of Compound (C15), except CDN intermediate (CDNI-12) was used in place of CDN intermediate (CDNI-23) and the C18 column was eluted with 5-50% acetonitrile-H₂O (aqueous phase containing 10 mM Et₃N HOAc). Fractions containing the desired product were concentrated to obtain Compound (C38) as Et₃N salt (11.6 mg, 88% yield) as Et₃N salt. LCMS M/2+1=676.8, tr=0.742 min. ¹H NMR (500 MHz, DMSO-d₆) δ 10.80 (s, 1H), 9.99 (s, 1H), 9.37 (s, 1H), 8.97 (s, 1H), 8.68 (s, 1H), 8.23 (s, 1H), 8.13 (d, J=7.5 Hz, 1H), 8.04 (d, J=8.4 Hz, 1H), 7.62 (t, J=10.0 Hz, 2H), 7.44 (s, 2H), 7.34 (t, J=9.9 Hz, 2H), 7.03 (s, 1H), 6.27 (d, J=8.8 Hz, 1H), 6.17 (d, J=8.8 Hz, 1H), 6.02 (s, 1H), 5.72-5.55 (m, 1H), 5.55-5.39 (m, 3H), 5.05 (s, 1H), 4.54 (ddd, J=27.3, 20.2, 2.4 Hz, 2H), 4.41 (td, J=8.1, 5.2 Hz, 1H), 4.31 (s, 2H), 4.19 (dd, J=8.5, 6.7 Hz, 1H), 4.05-3.91 (m, 3H), 3.72-3.60 (m, 1H), 3.59 (d, J=5.9 Hz, 2H), 3.11-3.02 (m, 1H), 3.00 (d, J=9.6 Hz, 3H), 2.80 (qd, J=13.5, 6.4 Hz, 16H), 2.52-2.42 (m, 1H), 1.94 (s, 3H), 1.73 (s, 1H), 1.69-1.57 (m, 1H), 1.52-1.34 (m, 2H), 1.02 (t, J=7.2 Hz, 20H), 0.86 (dd, J=15.8, 6.8 Hz, 5H).

### Example 3-39: Synthesis of Compound 39 (C39)

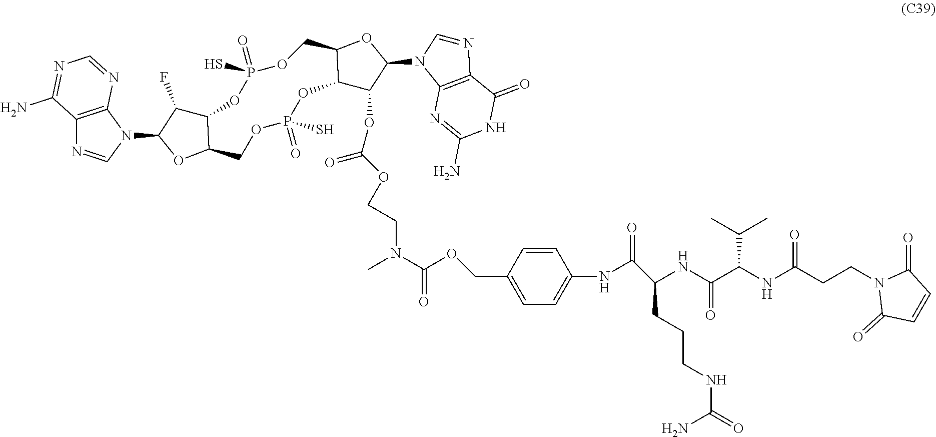 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(N)ncnc65)[C@H](F)[C@@H]4O[P@@](=O)(S)OC[C@H]3O[C@H]2n2cnc3c(=O)[nH]c(N)nc32)cc1]

Compound (C39) was synthesized using similar methods describe for the synthesis of Compound (C18), except CDN intermediate (CDNI-24) was used in place of CDN intermediate (CDNI-3) and the C18 column was eluted with 5-40% acetonitrile-H₂O (aqueous phase containing 10 mM Et₃N HOAc). Fractions containing the desired product were concentrated to obtain Compound (C39) as Et₃N salt: (4.9 mg, 41.6% yield). LCMS M/2+1=683.8, tr=0.709 min. ¹H NMR (500 MHz, DMSO-d₆) δ 9.99 (s, 1H), 8.35 (s, 1H), 8.21 (s, 1H), 8.17-7.99 (m, 3H), 7.68-7.52 (m, 2H), 7.33 (s, 5H), 7.03 (s, 2H), 6.57 (s, 2H), 6.30 (d, J=16.6 Hz, 1H), 6.02 (dd, J=55.5, 30.4 Hz, 2H), 5.60 (dd, J=52.2, 3.8 Hz, 1H), 5.42 (d, J=30.9 Hz, 3H), 5.01 (d, J=12.8 Hz, 2H), 4.39 (d, J=12.6 Hz, 2H), 4.30 (d, J=10.7 Hz, 4H), 4.27-4.06 (m, 4H), 3.92-3.74 (m, 2H), 3.69-3.50 (m, 3H), 3.14-2.83 (m, 5H), 2.69 (q, J=7.2 Hz, 33H), 1.86-1.56 (m, 1H), 1.56-1.31 (m, 2H), 1.05 (t, J=7.2 Hz, 44H), 0.86 (dd, J=15.5, 6.8 Hz, 6H).

### Example 3-40: Synthesis of Compound 40 (C40)

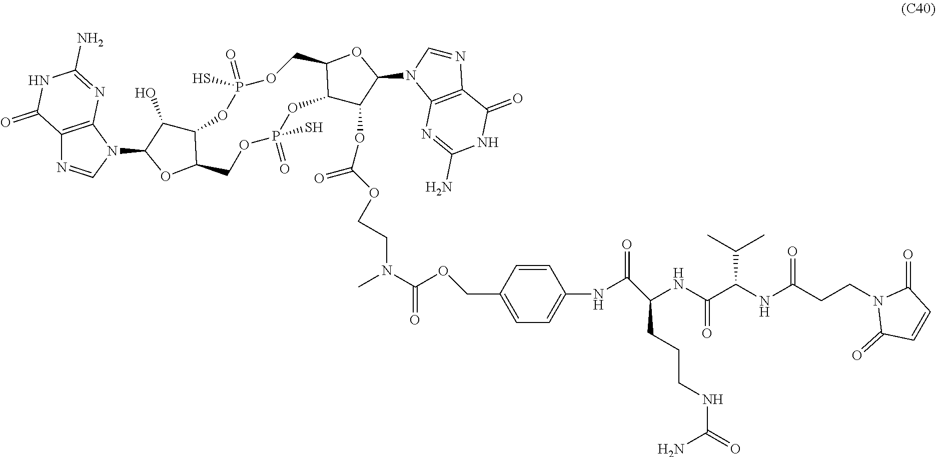 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(=O)[nH]c(N)nc65)[C@H](O)[C@@H]4O[P@](=O)(S)OC[C@H]3O[C@H]2n2cnc3c(=O)[nH]c(N)nc32)cc1]

Compound (C40) was synthesized using similar methods describe for the synthesis of Compound (C18), except CDN intermediate (CDNI-25) was used in place of CDN intermediate (CDNI-3). Compound (C40) as Et₃N salt: (8.0 mg, 74% yield). LCMS M/2+1=690.8, tr=0.771 min. ¹H NMR (500 MHz, DMSO-d₆) δ 10.02 (s, 1H), 8.14 (d, J=7.5 Hz, 1H), 8.04 (t, J=8.9 Hz, 3H), 7.63 (d, J=8.0 Hz, 2H), 7.33 (d, J=9.4 Hz, 2H), 7.03 (s, 2H), 6.71 (d, J=67.7 Hz, 5H), 6.03 (s, 2H), 5.78 (d, J=7.4 Hz, 1H), 5.59 (s, 1H), 5.45 (s, 2H), 5.15 (dt, J=9.2, 4.2 Hz, 1H), 5.06-4.83 (m, 3H), 4.58 (t, J=6.3 Hz, 1H), 4.42 (d, J=6.6 Hz, 1H), 4.33-4.09 (m, 6H), 4.06-3.86 (m, 2H), 3.65 (td, J=8.1, 6.7 Hz, 1H), 3.15-2.82 (m, 4H), 2.66 (q, J=7.2 Hz, 33H), 1.80-1.54 (m, 1H), 1.54-1.34 (m, 2H), 1.04 (t, J=7.2 Hz, 45H), 0.86 (dd, J=16.3, 6.8 Hz, 5H).

### Example 3-41: Synthesis of Compound 41 (C41)

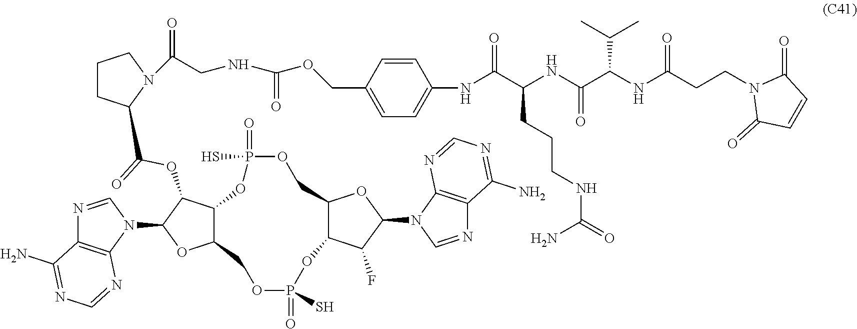 [SMILES: CC(C)[C@H](NC(=O)CCN1C(=O)C=CC1=O)C(=O)N[C@@H](CCCNC(N)=O)C(=O)Nc1ccc(COC(=O)NCC(=O)N2CCC[C@@H]2C(=O)O[C@@H]2[C@@H]3O[P@](=O)(S)OC[C@H]4O[C@@H](n5cnc6c(N)ncnc65)[C@H](F)[C@@H]4O[P@@](=O)(S)OC[C@H]3O[C@H]2n2cnc3c(N)ncnc32)cc1]

Compound (C41) was synthesized using similar methods describe for the synthesis of Compound (C18), except CDN intermediate (CDNI-26) TEA salt was used in place of CDN intermediate (CDNI-3) and Linker intermediate (LI-9) was used in place of Linker intermediate (LI-3). Fractions containing the desired product were combined and lyophilized to obtain Compound (C41) as Et₃N salt (2.3 mg, 11% yield). LCMS M/2+1=702.3, tr=0.691 min.

### Example 3-42: Synthesis of the Mixture of Compound 42a (C42a) and Compound 42b (C42b)

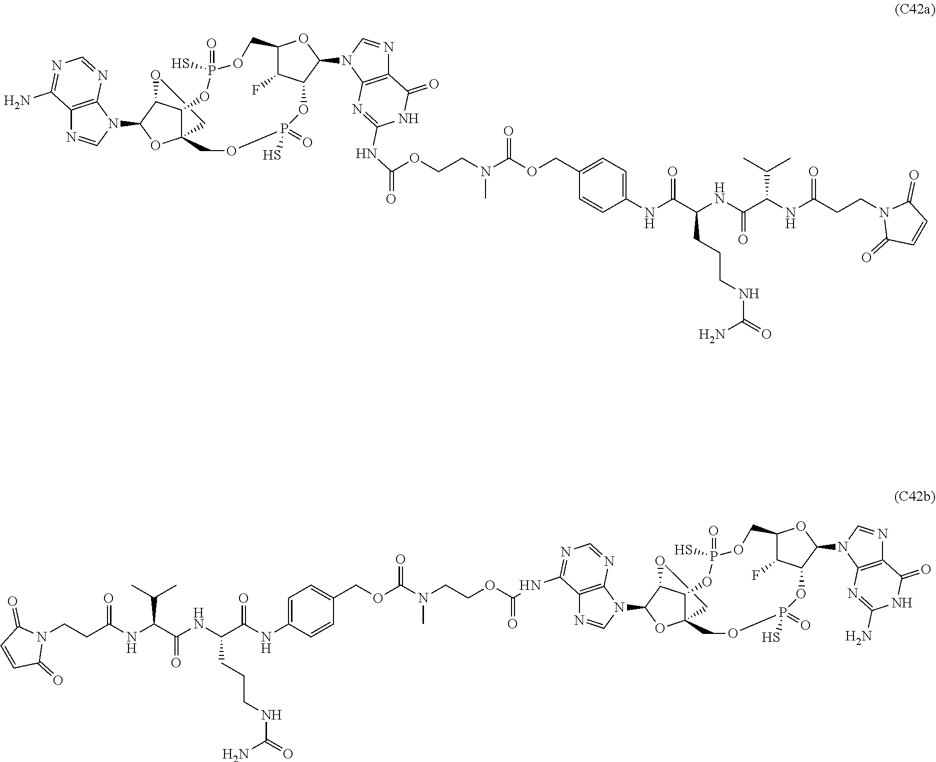 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2nc3c(ncn3[C@@H]3O[C@@H]4CO[P@@](=O)(S)O[C@H]5[C@H]6OC[C@]5(CO[P@@](=O)(S)O[C@@H]3[C@@H]4F)O[C@H]6n3cnc4c(N)ncnc43)c(=O)[nH]2)cc1 ; CC(C)[C@H](NC(=O)CCN1C(=O)C=CC1=O)C(=O)N[C@@H](CCCNC(N)=O)C(=O)Nc1ccc(COC(=O)N(C)CCOC(=O)Nc2ncnc3c2ncn3[C@@H]2O[C@@]34CO[C@@H]2[C@@H]3O[P@](=O)(S)OC[C@H]2O[C@@H](n3cnc5c(=O)[nH]c(N)nc53)[C@H](O[P@](=O)(S)OC4)[C@@H]2F)cc1]

The mixture of Compound (C42a) and Compound (C42b) was synthesized using similar methods describe for the synthesis of Compound (C18), except the mixture of CDN intermediate (CDNI-27a) and CDN intermediate (CDNI-27b) was used in place of CDN intermediate (CDNI-3) and the C18 column was eluted with 5-40% acetonitrile-H₂O (aqueous phase containing 10 mM Et₃N HOAc). The mixture of Compound (C42a) and Compound (C42b) was obtained as Et₃N salt (2.0 mg, 33% yield). LCMS M/2+1=689.8, tr=0.694 min.

### Example 3-43: Synthesis of Compound 43 (C43)

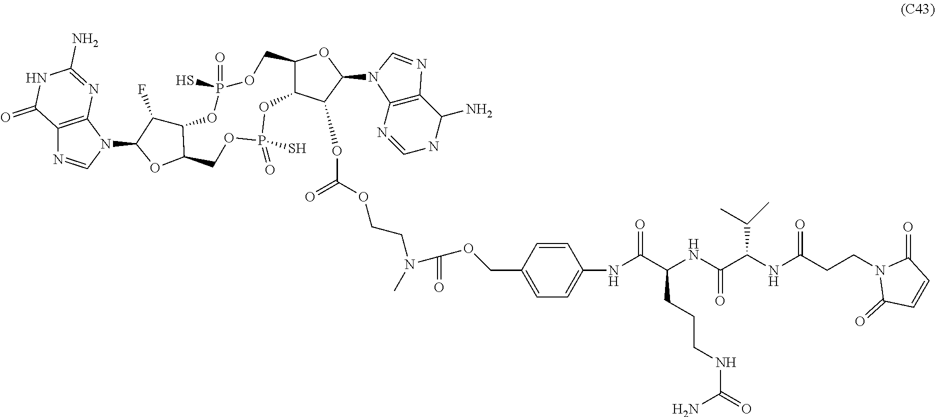 [SMILES: CC(C)[C@H](NC(=O)CCN1C(=O)C=CC1=O)C(=O)N[C@@H](CCCNC(N)=O)C(=O)Nc1ccc(COC(=O)N(C)CCOC(=O)O[C@@H]2[C@@H]3O[P@](=O)(S)OC[C@H]4O[C@@H](n5cnc6c(=O)[nH]c(N)nc65)[C@H](F)[C@@H]4O[P@@](=O)(S)OC[C@H]3O[C@H]2n2cnc3c2N=CNC3N)cc1]

Compound (C43) was synthesized using similar methods describe for the synthesis of Compound (C18), except CDN intermediate (CDNI-28) TEA salt was used in place of CDN intermediate (CDNI-3). Fractions containing the desired product were combined and lyophilized to obtain Compound (C43) as Et₃N salt (3.3 mg, 31.1% yield). LCMS M/2+1=683.8, tr=0.813 min.

### Example 3-44: Synthesis of a Mixture of Compound 44a (C44a) and Compound 44b (C44b)

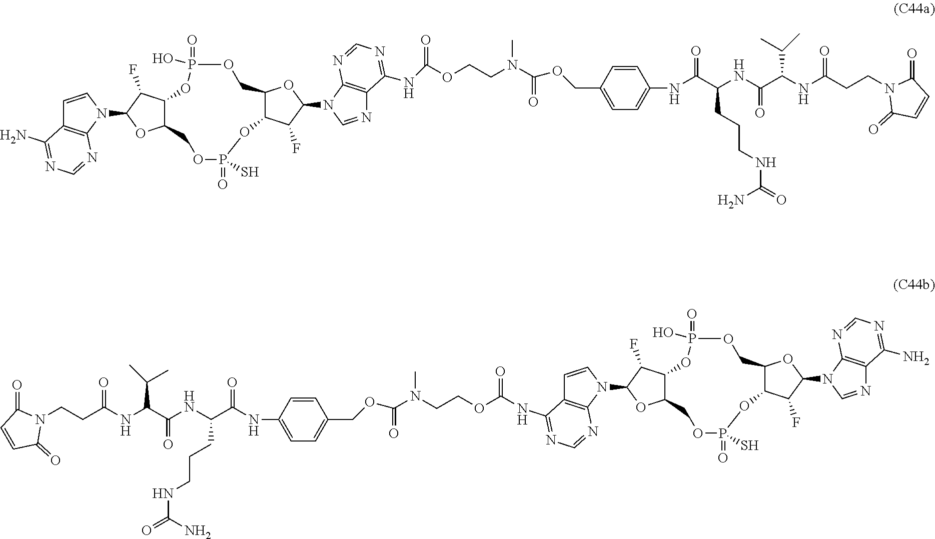

Compound (C1) (20 mg, 0.013 mmol) was dissolved in 3:7 MeOH and DMSO (1 ml) and maintained at rt for 1 month. The mixture was purified by reverse phase ISCO using 50 g C18 aq column, eluted with 5-40% ACN-water with 0.05% TFA. Fractions containing Compound (C44a) and Compound (C44b) were isolated and lyophilized to obtain the mixture of Compound (C44a) and Compound (C44b) as TFA salt (4.5 mg, 21% yield). LCMS M/2+1=668.8, tr=0.694 min.

### Example 3-45: Synthesis of Compound 45 (C45)

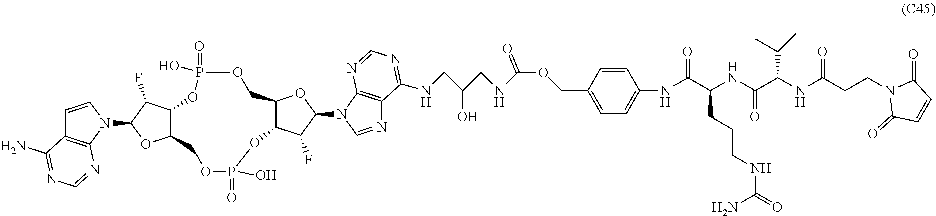 [SMILES: CC(C)[C@H](NC(=O)CCN1C(=O)C=CC1=O)C(=O)N[C@@H](CCCNC(N)=O)C(=O)Nc1ccc(COC(=O)NCC(O)CNc2ncnc3c2ncn3[C@@H]2O[C@@H]3COP(=O)(O)O[C@H]4[C@@H](F)[C@H](n5ccc6c(N)ncnc65)O[C@@H]4COP(=O)(O)O[C@H]3[C@H]2F)cc1]

Compound (C45) was synthesized using similar methods describe for the synthesis of Compound (C18), except CDN intermediate (CDNI-29) TEA salt was used in place of CDN intermediate (CDNI-3). Fractions containing the desired product were combined and lyophilized to obtain Compound (C45) as Et₃N salt (7.2 mg, 38% yield). LCMS M+1=1292.1, tr=0.631 min.

### Example 4: Generation of Anti-DC-SIGN Antibodies

### Generation of Expression Constructs for Human and Cynomolous Monkey DC-SIGN

Full length human DC-SIGN DNA (SEQ ID NO: 306) was synthesized based on amino acid sequences from the Uniprot databases (Q9NNX6, SEQ ID NO:303), the cyno DC-SIGN DNA (SEQ ID NO: 312) was synthesized based on cyno DC-SIGN amino acid sequence (SEQ ID NO: 311). All synthesized DNA fragments were cloned into appropriate expression vectors.

TABLE 21
Amino Acid and Nucleotide Sequence Information for DC-SIGN proteins

| | | |
|---|---|---|
| Human DC- | MSDS KEPRLQQLGL LEEEQLRGLG | SEQ ID |
| SIGN | FRQTRGYKSL AGCLGHGPLV LQLLSFTLLA GLLVQVSKVP SSISQEQSRQ | NO: 303 |
| Full length AA | DAIYQNLTQL KAAVGELSEK SKLQEIYQEL TQLKAAVGEL PEKSKLQEIY | |
| | QELTRLKAAV GELPEKSKLQ EIYQELTWLK AAVGELPEKS KMQEIYQELT | |
| | RLKAAVGELP EKSKQQEIYQ ELTRLKAAVG ELPEKSKQQE IYQELTRLKA | |
| | AVGELPEKSK QQEIYQELTQ LKAAVERLCH PCPWEWTFFQ GNCYFMSNSQ | |
| | RNWHDSITAC KEVGAQLVVI KSAEEQNFLQ LQSSRSNRFT WMGLSDLNQE | |
| | GTWQWVDGSP LLPSFKQYWN RGEPNNVGEE DCAEFSGNGW NDDKCNLAKF | |
| | WICKKSAASC SRDEEQFLSP APATPNPPPA | |
| Full length | AT GAGTGACTCC AAGGAACCAA | SEQ ID |
| DNA | GACTGCAGCA GCTGGGCCTC CTGGAGGAGG AACAGCTGAG AGGCCTTGGA | NO: 306 |
| | TTCCGACAGA CTCGAGGATA CAAGAGCTTA GCAGGGTGTC TTGGCCATGG | |
| | TCCCCTGGTG CTGCAACTCC TCTCCTTCAC GCTCTTGGCT GGGCTCCTTG | |
| | TCCAAGTGTC CAAGGTCCCC AGCTCCATAA GTCAGGAACA ATCCAGGCAA | |
| | GACGCGATCT ACCAGAACCT GACCCAGCTT AAAGCTGCAG TGGGTGAGCT | |
| | CTCAGAGAAA TCCAAGCTGC AGGAGATCTA CCAGGAGCTG ACCCAGCTGA | |
| | AGGCTGCAGT GGGTGAGCTT CCAGAGAAAT CTAAGCTGCA GGAGATCTAC | |
| | CAGGAGCTGA CCCGGCTGAA GGCTGCAGTG GGTGAGCTTC CAGAGAAATC | |
| | TAAGCTGCAG GAGATCTACC AGGAGCTGAC CTGGCTGAAG GCTGCAGTGG | |
| | GTGAGCTTCC AGAGAAATCT AAGATGCAGG AGATCTACCA GGAGCTGACT | |
| | CGGCTGAAGG CTGCAGTGGG TGAGCTTCCA GAGAAATCTA AGCAGCAGGA | |
| | GATCTACCAG GAGCTGACCC GGCTGAAGGC TGCAGTGGGT GAGCTTCCAG | |
| | AGAAATCTAA GCAGCAGGAG ATCTACCAGG AGCTGACCCG GCTGAAGGCT | |
| | GCAGTGGGTG AGCTTCCAGA GAAATCTAAG CAGCAGGAGA TCTACCAGGA | |
| | GCTGACCCAG CTGAAGGCTG CAGTGGAACG CCTGTGCCAC CCCTGTCCCT | |
| | GGGAATGGAC ATTCTTCCAA GGAAACTGTT ACTTCATGTC TAACTCCCAG | |
| | CGGAACTGGC ACGACTCCAT CACCGCCTGC AAAGAAGTGG GGGCCCAGCT | |
| | CGTCGTAATC AAAAGTGCTG AGGAGCAGAA CTTCCTACAG CTGCAGTCTT | |
| | CCAGAAGTAA CCGCTTCACC TGGATGGGAC TTTCAGATCT AAATCAGGAA | |
| | GGCACGTGGC AATGGGTGGA CGGCTCACCT CTGTTGCCCA GCTTCAAGCA | |
| | GTATTGGAAC AGAGGAGAGC CCAACAACGT TGGGGAGGAA GACTGCGCGG | |
| | AATTTAGTGG CAATGGCTGG AACGACGACA AATGTAATCT TGCCAAATTC | |
| | TGGATCTGCA AAAAGTCCGC AGCCTCCTGC TCCAGGGATG AAGAACAGTT | |
| | TCTTTCTCCA GCCCCTGCCA CCCCAAACCC CCCTCCTGCG | |
| ECD AA | KV PSSISQEQSR QDAIYQNLTQ LKAAVGELSE | SEQ ID |
| | KSKLQEIYQE LTQLKAAVGE LPEKSKLQEI YQELTRLKAA VGELPEKSKL | NO: 307 |
| | QEIYQELTWL KAAVGELPEK SKMQEIYQEL TRLKAAVGEL PEKSKQQEIY | |
| | QELTRLKAAV GELPEKSKQQ EIYQELTRLK AAVGELPEKS KQQEIYQELT | |
| | QLKAAVERLC HPCPWEWTFF QGNCYFMSNS QRNWHDSITA CKEVGAQLVV | |
| | IKSAEEQNFL QLQSSRSNRF TWMGLSDLNQ EGTWQWVDGS PLLPSFKQYW | |
| | NRGEPNNVGE EDCAEFSGNG WNDDKCNLAK FWICKKSAAS CSRDEEQFLS | |
| | PAPATPNPPP A | |
| ECD DNA | AAGGTCCCCA GCTCCATAAG TCAGGAACAA TCCAGGCAAG ACGCGATCTA | SEQ ID |
| | CCAGAACCTG ACCCAGCTTA AAGCTGCAGT GGGTGAGCTC TCAGAGAAAT | NO: 308 |
| | CCAAGCTGCA GGAGATCTAC CAGGAGCTGA CCCAGCTGAA GGCTGCAGTG | |
| | GGTGAGCTTC CAGAGAAATC TAAGCTGCAG GAGATCTACC AGGAGCTGAC | |
| | CCGGCTGAAG GCTGCAGTGG GTGAGCTTCC AGAGAAATCT AAGCTGCAGG | |
| | AGATCTACCA GGAGCTGACC TGGCTGAAGG CTGCAGTGGG TGAGCTTCCA | |
| | GAGAAATCTA AGATGCAGGA GATCTACCAG GAGCTGACTC GGCTGAAGGC | |
| | TGCAGTGGGT GAGCTTCCAG AGAAATCTAA GCAGCAGGAG ATCTACCAGG | |
| | AGCTGACCCG GCTGAAGGCT GCAGTGGGTG AGCTTCCAGA GAAATCTAAG | |
| | CAGCAGGAGA TCTACCAGGA GCTGACCCGG CTGAAGGCTG CAGTGGGTGA | |
| | GCTTCCAGAG AAATCTAAGC AGCAGGAGAT CTACCAGGAG CTGACCCAGC | |
| | TGAAGGCTGC AGTGGAACGC CTGTGCCACC CCTGTCCCTG GGAATGGACA | |
| | TTCTTCCAAG GAAACTGTTA CTTCATGTCT AACTCCCAGC GGAACTGGCA | |
| | CGACTCCATC ACCGCCTGCA AAGAAGTGGG GGCCCAGCTC GTCGTAATCA | |
| | AAAGTGCTGA GGAGCAGAAC TTCCTACAGC TGCAGTCTTC CAGAAGTAAC | |
| | CGCTTCACCT GGATGGGACT TTCAGATCTA AATCAGGAAG GCACGTGGCA | |
| | ATGGGTGGAC GGCTCACCTC TGTTGCCCAG CTTCAAGCAG TATTGGAACA | |
| | GAGGAGAGCC CAACAACGTT GGGGAGGAAG ACTGCGCGGA ATTTAGTGGC | |
| | AATGGCTGGA ACGACGACAA ATGTAATCTT GCCAAATTCT GGATCTGCAA | |
| | AAAGTCCGCA GCCTCCTGCT CCAGGGATGA AGAACAGTTT CTTTCTCCAG | |
| | CCCCTGCCAC CCCAAACCCC CCTCCTGCG | |
| CRD AA | ER LCHPCPWEWT FFQGNCYFMS NSQRNWHDSI | SEQ ID |
| | TACKEVGAQL VVIKSAEEQN FLQLQSSRSN RFTWMGLSDL NQEGTWQWVD | NO: 309 |
| | GSPLLPSFKQ YWNRGEPNNV GEEDCAEFSG NGWNDDKCNL AKFWICKKSA | |
| | ASCSRDEEQF LSPAPATPNP PPA | |
| CRD DNA | GAACGCCTGT GCCACCCCTG TCCCTGGGAA TGGACATTCT TCCAAGGAAA | SEQ ID |
| | CTGTTACTTC ATGTCTAACT CCCAGCGGAA CTGGCACGAC TCCATCACCG | NO: 310 |
| | CCTGCAAAGA AGTGGGGGCC CAGCTCGTCG TAATCAAAAG TGCTGAGGAG | |
| | CAGAACTTCC TACAGCTGCA GTCTTCCAGA AGTAACCGCT TCACCTGGAT | |
| | GGGACTTTCA GATCTAAATC AGGAAGGCAC GTGGCAATGG GTGGACGGCT | |
| | CACCTCTGTT GCCCAGCTTC AAGCAGTATT GGAACAGAGG AGAGCCCAAC | |
| | AACGTTGGGG AGGAAGACTG CGCGGAATTT AGTGGCAATG GCTGGAACGA | |
| | CGACAAATGT AATCTTGCCA AATTCTGGAT CTGCAAAAAG TCCGCAGCCT | |
| | CCTGCTCCAG GGATGAAGAA CAGTTTCTTT CTCCAGCCCC TGCCACCCCA | |
| | AACCCTCCTC CTGCG | |
| Cyno DC-SIGN | MSDSKEPRLQ QLDLLEEEQL GGVGFRQTRG YKSLAGCLGH GPLVLQLLSF | SEQ ID |
| Full length AA | TLLAGLLVQV SKVPSSLSQG QSKQDAIYQN LTQLKVAVSE LSEKSKQQEI | NO: 311 |
| | YQELTRLKAA VGELPEKSKQ QEIYEELTRL KAAVGELPEK SKLQEIYQEL | |
| | TRLKAAVGEL PEKSKQQEIY QELSRLKAAV GDLPEKSKQQ EIYQKLTQLK | |
| | AAVDGLPDRS KQQEIYQELI QLKAAVDLEG WTDTGIWTTS SEPSPDRPPP | |
| | TERLCHPCPW EWTFFQGNCY FMSNSQRNWH DSITACQEVG AQLVVIKSAE | |
| | EQNFLQLQSS RSNRFTWMGL SDLNHEGTWQ WVDGSPLLPS FKQYWNKGEP | |
| | NNVGEEDCAE FSGNGWNDDK CNLAKFWICK KSAASCSGDE ERLLSPAPTT | |
| | PNPPPE | |
| Full length | atgtcggactcgaaggaaccaagactgcagcaactcgacctccttgaagaagaacagctcgg | SEQ ID |
| DNA | cggagtgggattccggcagaccaggggttacaagagcctggccggttgcctgggtcacggcc | NO: 312 |
| | ctttggtgcttcagctgctgtcgttcaccctgctggccggactgcttgtgcaagtctccaaa | |
| | gtcccgtcctcgctgagccaggggcagtccaagcaggacgcgatctaccaaaacctgacaca | |
| | gctcaaggtggccgtgtcagagctgtccgagaagtcgaagcagcaagagatctaccaagagt | |
| | tgacgcgactcaaagcagccgtgggcgaacttcccgagaagtcaaagcagcaggaaatctac | |
| | gaggaattgacccgcctgaaggccgccgtgggagagctgccagaaaagtcgaagctgcagga | |
| | gatataccaagaactcacccggctcaaggccgctgtgggagaactgccggagaagtccaaac | |
| | aacaggaaatctaccaggaactgagcagactcaaggcagccgtcggcgatctccccgaaaag | |
| | tctaaacagcaggagatctatcagaagctgactcagctgaaggcggccgtggacgggctgcc | |
| | cgatcggtccaagcaacaggaaatctaccaggagctgatccaactgaaggctgccgtggacc | |
| | tggaagggtggactgacaccgggatttggactacctcatcggaaccgagccctgatcgccct | |
| | ccgcctaccgagaggttgtgtcacccgtgcccatgggagtggacgttcttccaaggaaactg | |
| | ttactttatgagcaacagccagcggaattggcacgattccattaccgcgtgccaggaagtgg | |
| | gcgcccagctggtcgtgatcaagtccgcggaggagcagaacttcctgcagctccagagcagc | |
| | cggtccaaccgcttcacctggatgggcctctccgacctgaaccatgagggaacttggcagtg | |
| | ggtggacggttccccgctgctgccctcattcaagcagtactggaacaagggagaaccgaaca | |
| | acgtcggagaggaagattgcgccgagttttccgggaacggatggaacgacgacaagtgcaat | |
| | ctggccaagttctggatttgcaagaagtccgctgcatcctgctcgggcgacgaggagcgcct | |
| | gctgtcccccgcgcccaccacccctaaccctcccccggaatgatag | |
| ECD AA | QPSKQD AIYQNLTQLK VAVSELSEKS | SEQ ID |
| | KQQEIYQELT RLKAAVGELP EKSKQQEIYE ELTRLKAAVG ELPEKSKLQE | NO: 313 |
| | IYQELTRLKA AVGELPEKSK QQEIYQELSR LKAAVGDLPE KSKQQEIYQK | |
| | LTQLKAAVDG LPDRSKQQEI YQELIQLKAA VDLEGWTDTG IWTTSSEPSP | |
| | DRPPPTERLC HPCPWEWTFF QGNCYFMSNS QRNWHDSITA CQEVGAQLVV | |
| | IKSAEEQNFL QLQSSRSNRF TWMGLSDLNH EGTWQWVDGS PLLPSFKQYW | |
| | NKGEPNNVGE EDCAEFSGNG WNDDKCNLAK FWICKKSAAS CSGDEERLLS | |
| | PAPTTPNPPP | |
| ECD DNA | TC CAAGCAGGAC GCGATCTACC | SEQ ID |
| | AAAACCTGAC ACAGCTCAAG GTGGCCGTGT CAGAGCTGTC CGAGAAGTCG | NO: 314 |
| | AAGCAGCAAG AGATCTACCA AGAGTTGACG CGACTCAAAG CAGCCGTGGG | |
| | CGAACTTCCC GAGAAGTCAA AGCAGCAGGA AATCTACGAG GAATTGACCC | |
| | GCCTGAAGGC CGCCGTGGGA GAGCTGCCAG AAAAGTCGAA GCTGCAGGAG | |
| | ATATACCAAG AACTCACCCG GCTCAAGGCC GCTGTGGGAG AACTGCCGGA | |
| | GAAGTCCAAA CAACAGGAAA TCTACCAGGA ACTGAGCAGA CTCAAGGCAG | |
| | CCGTCGGCGA TCTCCCCGAA AAGTCTAAAC AGCAGGAGAT CTATCAGAAG | |
| | CTGACTCAGC TGAAGGCGGC CGTGGACGGG CTGCCCGATC GGTCCAAGCA | |
| | ACAGGAAATC TACCAGGAGC TGATCCAACT GAAGGCTGCC GTGGACCTGG | |
| | AAGGGTGGAC TGACACCGGG ATTTGGACTA CCTCATCGGA ACCGAGCCCT | |
| | GATCGCCCTC CGCCTACCGA GAGGTTGTGT CACCCGTGCC CATGGGAGTG | |
| | GACGTTCTTC CAAGGAAACT GTTACTTTAT GAGCAACAGC CAGCGGAATT | |
| | GGCACGATTC CATTACCGCG TGCCAGGAAG TGGGCGCCCA GCTGGTCGTG | |
| | ATCAAGTCCG CGGAGGAGCA GAACTTCCTG CAGCTCCAGA GCAGCCGGTC | |
| | CAACCGCTTC ACCTGGATGG GCCTCTCCGA CCTGAACCAT GAGGGAACTT | |
| | GGCAGTGGGT GGACGGTTCC CCGCTGCTGC CCTCATTCAA GCAGTACTGG | |
| | AACAAGGGAG AACCGAACAA CGTCGGAGAG GAAGATTGCG CCGAGTTTTC | |
| | CGGGAACGGA TGGAACGACG ACAAGTGCAA TCTGGCCAAG TTCTGGATTT | |
| | GCAAGAAGTC CGCTGCATCC TGCTCGGGCG ACGAGGAGCG CCTGCTGTCC | |
| | CCCGCGCCCA CCACCCCTAA CCCTCCCCCG GAA | |
| CRD AA | OPERLC HPCPWEWTFF QGNCYFMSNS | SEQ ID |
| | QRNWHDSITA CQEVGAQLVV IKSAEEQNFL QLQSSRSNRF TWMGLSDLNH | NO: 315 |
| | EGTWQWVDGS PLLPSFKQYW NKGEPNNVGE EDCAEFSGNG WNDDKCNLAK | |
| | FWICKKSAAS CSGDEERLLS PAPTTPNPPP E | |
| CRD DNA | GA GAGGTTGTGT CACCCGTGCC | SEQ ID |
| | CATGGGAGTG GACGTTCTTC CAAGGAAACT GTTACTTTAT GAGCAACAGC | NO: 316 |
| | CAGCGGAATT GGCACGATTC CATTACCGCG TGCCAGGAAG TGGGCGCCCA | |
| | GCTGGTCGTG ATCAAGTCCG CGGAGGAGCA GAACTTCCTG CAGCTCCAGA | |
| | GCAGCCGGTC CAACCGCTTC ACCTGGATGG GCCTCTCCGA CCTGAACCAT | |
| | GAGGGAACTT GGCAGTGGGT GGACGGTTCC CCGCTGCTGC CCTCATTCAA | |
| | GCAGTACTGG AACAAGGGAG AACCGAACAA CGTCGGAGAG GAAGATTGCG | |
| | CCGAGTTTTC CGGGAACGGA TGGAACGACG ACAAGTGCAA TCTGGCCAAG | |
| | TTCTGGATTT GCAAGAAGTC CGCTGCATCC TGCTCGGGCG ACGAGGAGCG | |
| | CCTGCTGTCC CCCGCGCCCA CCACCCCTAA CCCTCCCCCG GAA | |
| Human DC- | KV PSSISQEQSR QDAIYQNLTQ LKAAVGELSE | SEQ ID |
| SIGN ECD- | KSKLQEIYQE LTQLKAAVGE LPEKSKLQEI YQELTRLKAA VGELPEKSKL | NO: 317 |
| AviHis | QEIYQELTWL KAAVGELPEK SKMQEIYQEL TRLKAAVGEL PEKSKQQEIY | |
| | QELTRLKAAV GELPEKSKQQ EIYQELTRLK AAVGELPEKS KQQEIYQELT | |
| | QLKAAVERLC HPCPWEWTFF QGNCYFMSNS QRNWHDSITA CKEVGAQLVV | |
| | IKSAEEQNFL QLQSSRSNRF TWMGLSDLNQ EGTWQWVDGS PLLPSFKQYW | |
| | NRGEPNNVGE EDCAEFSGNG WNDDKCNLAK FWICKKSAAS CSRDEEQFLS | |
| | PAPATPNPPP AGSGGGLNDI FEAQKIEWHE HHHHHH | |
| Human DC- | MQLLSCIALS LALVTNSTER LCHPCPWEWT FFQGNCYFMS NSQRNWHDSI | SEQ ID |
| SIGN CRD- | TACKEVGAQL VVIKSAEEQN FLQLQSSRSN RFTWMGLSDL NQEGTWQWVD | NO: 318 |
| AviHis | GSPLLPSFKQ YWNRGEPNNV GEEDCAEFSG NGWNDDKCNL AKFWICKKSA | |
| | ASCSRDEEQF LSPAPATPNP PPAGSGGGLN DIFEAQKIEW HEHHHHHH | |
| Human DC- | MKTFILLLWV LLLWVIFLLP GATAQPSKVP SSISQEQSRQ DAIYQNLTQL | SEQ ID |
| SIGN ECD- | KAAVGELSEK SKLQEIYQEL TQLKAAVGEL PEKSKLQEIY QELTRLKAAV | NO: 319 |
| FLAGHis | GELPEKSKLQ EIYQELTWLK AAVGELPEKS KMQEIYQELT RLKAAVGELP | |
| | EKSKQQEIYQ ELTRLKAAVG ELPEKSKQQE IYQELTRLKA AVGELPEKSK | |
| | QQEIYQELTQ LKAAVERLCH PCPWEWTFFQ GNCYFMSNSQ RNWHDSITAC | |
| | KEVGAQLVVI KSAEEQNFLQ LQSSRSNRFT WMGLSDLNQE GTWQWVDGSP | |
| | LLPSFKQYWN RGEPNNVGEE DCAEFSGNGW NDDKCNLAKF WICKKSAASC | |
| | SRDEEQFLSP APATPNPPPA DYKDDDDKHH HHHH | |

### Generation of Cell Lines Stably Expressing DC-SIGN

Stable full length DC-SIGN-expressing and full length L-SIGN expressing K562 cell lines were generated using retroviral transduction. HEK293T cells were co-transfected with a DC-SIGN retroviral expression vector and a pCL-10A1 packaging vector (Novus, USA, cat#NBP2-2942) using Fugene 6 transfection reagent (Promega, USA, cat# E2692) following manufacturer's recommendation. Cells were incubated in a 37° C. humidified CO₂ incubator and viral supernatant was collected 48 hours post-transfection. K562 cells were grown to near confluency. Viral transduction was performed by adding viral supernatant in the presence of 8 μg polybrene/ml (final concentration) (EMD Millipore, cat#TR-1003-G). Following incubation for 3-6 hours at 37° C., fresh media was added. Cells were then cultured under appropriate selection conditions to produce stable L-SIGN or DC-SIGN expressing cell lines.

Stable human DC-SIGN expressing and cynomolgus monkey DC-SIGN expressing CHO cell lines were generated using plasmid DNA. Proprietary CHO cells were nucleoporated with a human or cynomolgus monkey DC-SIGN gene in the pD649 expression vector (DNA2.0). Nucleoporation was performed using the Lonza SG Cell line 96-well Nucleoporation kit (Cat# V4SC-3096). Cells and plasmid DNA were mixed with SG buffer and supplement, following manufacturer's recommendation. The 96-well nucleoporation plate was placed in a Nucleofector™ 96-well Shuttle™ (Lonza) and processed using program CHO S (FF-137). Nucleoporated cells were allowed to sit for 30 min at RT before diluting. Viability and cell density measurements were performed using VICELL (Beckman Coulter). Cells were seeded into a 96-well plate at 40,000 cells/well into 100 uL of proprietary DM122 media and incubated at 37° C., 10% CO2 at 4 hrs after seeding, selection was added to the cells (4 ug/mL of puromycin (InvivoGen) for cynomolgus monkey and 100 nM methotrexate (Sigma) for human DC-SIGN). Every 7 days, cells were passed 1:5 into fresh selection media for 3 passages. Cells were expanded into shake flasks at 37° C., 10% CO₂ and kept at densities 0.1million cells/mL to 2 million cells/mL. After 4 weeks, cells were FACS sorted using a 2008 FACS Aria to obtain cell pools with high expression levels for both cell lines.

### Hybridoma Generation, Antibodies 282 and 1G12

Bcl-2 transgenic mice (C57BL/6-Tgn (bcl-2) 22 WEHI strain) were immunized with antigen using a procedure that calls for Repetitive Immunization at Multiple Sites (RIMMS) (Kilpatrick K E, et al., Hybridoma 16(4):381-9 (1997)). Briefly, mice were injected with 1-3 μg of DC-SIGN immunogen (Recombinant Human DC-SIGN/CD209 Fc Chimera Protein, CF, R&D systems Cat No: 161-DC-050) at 8 specific sites proximal to peripheral lymph nodes (PLNs). This procedure was repeated 8 times over a 12 day period. On Day 12, a test bleed was collected and the serum antibody titer was analyzed by FACS. Two days after the boost, a test bleed was collected and serum antibody titer was analyzed by FACS. In some instances, BALB/c mice were immunized subcutaneously with antigen once a month for 3 months followed by an intravenous boost. Two days after the boost, a test bleed was collected and serum antibody titer was analyzed by FACS. Spleens and pooled PLNs were removed from high titer mice. To harvest lymphocytes, spleens and PLNs were washed twice with DMEM, and then dissociated by passage through a 70 micron screen (Falcon #352350). The resulting lymphocytes were washed 2 additional times prior to fusion in Cytofusion media (BTXpress Cytofusion® Electroporation Medium cat#47001).

Ten days after fusion, hybridoma plates were screened for the presence of human DC-SIGN-specific antibodies using flow cytometry. To confirm specific binding of candidate antibodies to cell surface-expressed human DC-SIGN, three cell lines were used: human DC-SIGN stably overexpressing K562, human L-SIGN stably overexpressing K562 or parental K562. Cells were rinsed thoroughly with PBS. Cells were biotinylated and labeled with a fluorescent dye according to manufacturer's instructions (FluoReporter™ Cell-Surface Biotinylation Kit, Thermo Fisher Scientific Cat# F-20650; PE-Cy7 Streptavidin, ThermoFisher Scientific Cat# SA1012; APC Streptavidin, Biolegend Cat#405207; APC/Cy7 Streptavidin, Biolegend Cat#405208). Cells were resuspended at approximately 1×10⁶ cells/ml in FACS buffer (PBS with 2% FBS+0.1% NaN₃). In a 384-well plate, 20 μL of hybridoma supernatant was pre-seeded, and 20 μL of cell suspension was added. Cells were incubated for 1 hour at 4° C., washed twice with cold FACS buffer, and resuspended in 20 μL of FACS buffer containing secondary antibody at a 1:400 dilution (Goat anti-mouse IgG BV421, Sirigen, custom order). After additional incubation for 45 min at 4° C., cells were washed twice with FACS buffer and resuspended in 20 μL of FACS buffer with 2 μg/ml propidium iodide (Sigma Aldrich Cat# P4864). Geometric mean fluorescence intensity was calculated on live single cells using FlowJo™ software.

### Hybridoma Generation 2, Antibodies 960K03, 958N02, 956P16, 952G04, 952D15, 914M09, 906C18, 956E02, 550E03, 942K11

Ablexis Alivamab Kappa (AMM-K) and Lambda (AMM-L) mice were immunized with antigen using a procedure that calls for Repetitive Immunization at Multiple Sites (RIMMS) (Kilpatrick K E, et al., Hybridoma 16(4):381-9 (1997)). Briefly, mice were injected with 22.5 μg of full length ECD-AviHis (SEQ ID NO: 317) protein at 8 specific sites proximal to peripheral lymph nodes (PLNs). This procedure was repeated 8 times over a 20 day period. On Day 18, a test bleed was collected and the serum antibody titer was analyzed by FACS and ELISA prior to hybridoma fusion. To harvest lymphocytes, spleens and lymph nodes were mechanically dissociated in PBS, and then passaged through a 70 micron screen (Falcon #352350). RBCs were lysed using Red Blood Cell Lysing Buffer (SigmaR7757-100 ml) as per manufacturer's instructions. CD3 positive splenocytes were removed using micro bead magnetic columns from Miltenyi as per their instructions (Anti-IgM #130-047-301 and anti-CD3 #130-094-973). The resulting lymphocytes were washed 2 additional times prior to fusion in Electrofusion IsoOsmolar Buffer (Eppendorf, #4308 070 536).

For the fusion, F0 myeloma cells were mixed with lymphocytes at a 1:4 ratio. The cell mixture was centrifuged, suspended in Electrofusion IsoOsmolar Buffer and subsequently added to an electrofusion chamber (Harvard Apparatus Coaxial chamber 9ML Part #470020). Electrofusion was carried out per manufacturer's instructions using the CEEF-50B Hybrimune/Hybridoma system (Cyto Pulse Sciences, Inc). Fused cells were allowed to recover for 5 minutes in the chamber, diluted 1:10 in media without hypoxanthine-aminopterin-thymidine (HAT) [DMEM+20% FBS, 1% Penicillin-Streptomycin-Glutamine (PSG), 1× Non-Essential Amino Acids (NEAA), 0.5× Hybridoma Fusion and Cloning Supplement (Roche; HFCS) and placed at 37° C. and 5% CO₂ for one hour. Next, 4×HAT medium (DMEM+20% FBS, 1% PSG, 1×NEAA, 4×HAT, 0.5×HFCS) was added to bring the concentration of HAT to 1×, and the density was adjusted to 66,000 cells/ml. The cells were plated in 384-well plates at 60 μl/well.

### FACS Screening

Ten days after fusion, hybridoma plates were screened for the presence of human DC-SIGN-specific antibodies using flow cytometry. To confirm specific binding of candidate antibodies to cell surface-expressed human DC-SIGN, three cell lines were used: human DC-SIGN stably overexpressing CHO, cynomolgus DC-SIGN stably overexpressing CHO, and parental non-transfected CHO cells. Cells were rinsed thoroughly with PBS. Cells were biotinylated and labeled with a fluorescent dye according to manufacturer's instructions (FluoReporter™ Cell-Surface Biotinylation Kit, Thermo Fisher Scientific Cat# F-20650; PE-Cy7 Streptavidin, ThermoFisher Scientific Cat# SA1012; APC Streptavidin, Biolegend Cat#405207; APC/Cy7 Streptavidin, Biolegend Cat#405208). Cells were resuspended at approximately 1×10⁶ cells/ml in FACS buffer (PBS with 2% FBS+0.1% NaN₃). In a 384-well plate, 20 μL of hybridoma supernatant was pre-seeded, and 20 μL of cell suspension was added. Cells were incubated for 1 hour at 4° C., washed twice with cold FACS buffer, and resuspended in 20 μL of FACS buffer containing secondary antibody at a 1:400 dilution (Goat anti-mouse IgG BV421, Sirigen, custom order). After additional incubation for 45 min at 4° C., cells were washed twice with FACS buffer and resuspended in 20 μL of FACS buffer with 2 μg/ml propidium iodide (Sigma Aldrich Cat# P4864). Geometric mean fluorescence intensity was calculated on live single cells using FlowJo™ software.

Hits from the primary cell-based flow cytometry screen were confirmed in a secondary flow cytometry screen like above, but with two additional cell lines: human DC-SIGN stably overexpressing K562, and human L-SIGN stably overexpressing K562 cells. Hybridomas expressing antibodies that bound to both human DC-SIGN expressing CHO and human DC-SIGN expressing K562 cells, but not CHO parental cells or L-SIGN-K562 cells, were called positive. Positive cells were expanded for cryo preservation and also split into 45 mL protein production cultures in hybridoma serum-free medium with HT Media Supplement (50×) Hybri-Max™ (Sigma, cat# H0137) in CellStar® Autoflasks™ (Greiner Bio-One). Production cultures were maintained in a shaking incubator at 37° C. and 5% CO₂ for approximately 8 days. Cells were then pelleted, and supernatants were taken through purification over Protein G resin. Proteins were subsequently buffer exchanged into PBS using NAP-10™ columns (GE Healthcare).

### Antibody Sequencing and Vector Preparation

Variable region (VH and VL) DNA sequences of hybridomas were obtained for each of the selected hybridomas. Variable region DNA products from murine monoclonal antibodies 2B2 and 1G12 were amplified by rapid amplification of cDNA ends (RACE) from RNA obtained from each selected hybridoma cell line using standard methods. Variable region DNA products from monoclonal antibodies 960K03, 958N02, 956P16, 952G04, 952D15, 914M09, 906C18, 956E02, 550E03, 942K11 were amplified by PCR from selected hybridoma cell line using standard methods and pooled primers to signal peptide and constant regions of the antibody genes.

For preparation of recombinant antibodies, DNA sequences coding for the hybridoma VL and VH domain were subcloned into expression vectors containing the respective human heavy or light chain constant region sequences (IgG1, kappa). In some instances this resulted in chimeric antibody chains comprising a murine variable region and human constant region. In some instances this resulted in fully human antibody sequence. In some instances, expression vectors contained wild type human constant region sequences. In some instances, expression vectors contained human constant region sequences comprising site-specific cysteine mutations as has been described previously in WO 2014/124316 and WO 2015/138615. For example, cysteines were introduced at one or more of the following positions (all positions by EU numbering) in an anti-DC-SIGN antibody: (a) positions 152 and/or 375 of the antibody heavy chain, and (b) position 165 of the antibody light chain. In some instances, constant region sequences comprise mutations known in the art to alter binding to Fc-receptors (e.g., D265A/P329A mutations in the heavy chain) to include constructs having reduced Fc effector function. In some instances, expression vectors contain constant regions comprising combinations of the modifications described above. In some instances, expression vectors contained mouse constant region sequences (IgG2a, kappa), either wild-type or with one or more mutations analagous to those described above (e.g. E152C, A375C, D265A, P329A), resulting in fully mouse antibody sequences. Heavy and light chains were cloned into individual expression vectors to allow co-transfection.

### Humanization of Antibodies 282 and 1G12

Variable region constructs were designed for humanization and optimization of sequences (e.g., removal of post-translational modifications, non-preferred sites, etc.).

Corresponding DNA sequences coding for humanized VL and VH domains were ordered at GeneArt (Life Technologies Inc. Regensburg, Germany), including codon optimization for Cricetulus griseus. Sequences coding for VL and VH domains were subcloned from the GeneArt derived vectors into expression vectors suitable for protein production in mammalian cells as described above for parental sequences. In some instances, the expression vector for the heavy chain comprised a truncation resulting in expression of a Fab fragment, and in some instances this constant region sequence was modified with a site-specific cysteine mutation at position 152 as described above, and additionally in some instances there was a sequence encoding a His-tag fused to the C-terminus of the Fab heavy chain coding sequence. Heavy and light chains were cloned into individual expression vectors to allow co-transfection.

### Optimization of Antibodies 960K03, 958N02, 956P16, 952G04, 952D15

Variable region constructs were designed for optimization of sequences by removal of post-translational modifications, non-preferred sites etc. Substitutions were made by site directed mutagenesis using standard methods. Heavy and light chains were cloned into individual expression vectors to allow co-transfection.

### Antibody Production

Recombinant antibodies (IgG1, kappa) were produced by co-transfection of heavy chain and light chain vectors into Freestyle™ 293 expression cells (Invitrogen, USA) using standard methods known in the art and similar to those described previously in Meissner, et al., Biotechnol Bioeng. 75:197-203 (2001).

Following transfection, the cells were cultured for one to two weeks prior to antibody purification from supernatant.

Alternatively, recombinant antibodies were produced by co-transfection of heavy chain and light chain vectors into CHO cells using methods known in the art. Following transfection, the cells were kept in culture for up to two weeks prior to antibody purification from supernatant.

To generate stable cell lines for antibody production, vectors were co-transfected by nucleofection (Nucleofector™ 96-well Shuttle™; Lonza) into CHO cells using manufacturer's recommendations, and cultured under selection conditions for up to four weeks in shake flasks. Cells were harvested by centrifugation, and supernatant recovered for antibody purification.

Antibodies and antibody fragmentsy were purified using Pprotein A, Protein G or MabSelect SuRe (GE Healthcare Life Sciences) columns. Prior to loading the supernatant, the resin was equilibrated with PBS. Following binding of the sample, the column was washed with PBS, and the antibody was eluted with Thermo (Pierce) IgG Elution Buffer pH 2.8 (cat#21004). The eluate fractions were neutralized with sodium citrate tribasic dehydrate buffer, pH 8.5 (Sigma Aldrich cat# S4641-1 Kg). Buffer exchange was performed by dialyzing overnight or by NAP-10™ columns (GE Healthcare), typically into PBS, pH 7.2. In some instances, antibodies may be further purified. One example is to apply the antibody to a size exclusion chromatography (SEC) column such as one with Superdex™ 200 resin (GE Healthcare) and collect the peak corresponding to the monomer species.

### Summary of Antibodies

Table 8 sets forth the relevant sequence information for parental and humanized anti-DC-SIGN antibodies derived from murine hybridomas. Throughout this application, when describing the antibodies, the term “Hybridoma” is used interchangeably and may refer to the antibody that is derived from the hybridoma.

### Example 5: Biochemical Characterization of Antibodies

### Affinities of Anti-DC-SIGN Antibodies to DC-SIGN

The affinity of various antibodies and ADCs to DC-SIGN and its species orthologues was determined using FACS. Purified IgGs were titrated to determine EC50 values for binding to cell surface expressed DC-SIGN.

For this purpose, human DC-SIGN expressing or cynomolgus monkey DC-SIGN expressing stable CHO cell lines or K562 expressing DC-SIGN or K562 expressing L-SIGN cell lines were checked for density and viability using VICELL (Beckman Coulter), and washed once with 4° C. PBS. Cells were stained with DAPI (0.5 ug/mL) diluted in PBS for 30 min on ice. Cells were diluted into 4° C. FACS buffer (PBS, 10 mM EDTA, 2% FBS). 125 μl of cells were seeded (10,000 cells/well) into 96-well v-bottom plates (Nunc cat#442587) and centrifuged for 4 min at 1500 rpm at 4° C. Supernatant was removed. Cells were incubated with a serial dilution of each anti-DC-SIGN antibody in FACS buffer at concentrations ranging across several logs with a top concentration no higher than 50 μg/mL for 60 minutes at 4° C. Following incubation, cells were spun down (1500 rpm, 4 min, 4° C.) and washed two times with FACS buffer. A fluorophore-conjugated anti-hFc gamma-AF-647 (Southern Biotechnology) detection antibody was added at 1:400 and samples were incubated for 1 h on ice in the dark. Following incubation, FACS buffer was added, and the cells were spun down (1500 rpm, 4 min, 4° C.) and washed two times with FACS buffer. After the final wash, cells were resuspended in Fixative Buffer (Biolegend, 420801) and 90 μl of FACS buffer followed by readout on the flow cytometry machine (BD LSRFortessa Cell Analyzer; Cat #647177). Geometric Mean fluorescence intensity (MFI) of live, single cells was calculated in Flowjo 10.4.2 and exported into Graphpad Prism7 for EC50 determination.

Selectivity was assessed by measuring apparent binding affinities to isogenic cell pairs engineered to overexpress DC-SIGN as well as cell lines expressing DC-SIGN paralog L-SIGN. Anti-DC-SIGN antibodies bind in a specific manner to DC-SIGN expressing cells only, as shown in Table 22 below.

In a similar experiment the antibodies were tested for cross-reactivity using engineered isogenic matched cell line. All antibodies except 892D15 and 942K11 were found to specifically bind human and cynomolgus monkey DC-SIGN at similar apparent affinities, as shown in Table 22 below.

TABLE 22
Binding of Various Anti-DC-SIGN Antibodies to DC-SIGN and L-SIGN Expressing Cells

| | human DC-SIGN CHO | cyno DC-SIGN CHO | human DC-SIGN K562 | human L-SIGN K562 |
|---|---|---|---|---|
| Antibody Name | EC50 (ug/mL) ave. | EC50 (ug/mL) ave. | EC50 (ug/mL) | EC50 (ug/mL) |
| 2B2 Hz | 0.06 | 0.04 | 0.27 | >10 |
| 960K03 N92S | 0.06 | 0.08 | 0.049 | |
| 958N05 S93A | 0.13 | 0.16 | 0.060 | >10 |
| 960K03 N92Q | 0.08 | 0.04 | 0.021 | >10 |
| 952P16 N92Q | 0.06 | 0.04 | 0.024 | >10 |
| 952G04 N92Q | 0.07 | 0.02 | 0.017 | >10 |
| 2B2 Chimeric | 0.23 | 0.32 | 0.28 | >10 |
| 960K03 Parental | 0.10 | 0.02 | | |
| 958N05 Parental | 0.07 | 0.08 | 0.05 | |
| 952P16 Parental | 0.06 | 0.02 | 0.04 | |
| 952G04 Parental | 0.11 | 0.19 | 0.26 | |
| 892D15 Parental | 0.15 | 15.69 | >10 | |
| 914M09 Parental | 0.10 | 0.08 | 0.25 | >10 |
| 906C18 Parental | 0.21 | 0.77 | 1.72 | >10 |
| 956 E02 Parental | 0.12 | 0.13 | | |
| 942K11 Parental | 0.20 | 11.57 | | |
| 550 E03 Parental | 0.26 | 0.39 | 1.50 | >10 |
| 1G12 Hz | 0.07 | 0.06 | 0.021 | >10 |
| 1G12 mouse | | | | |
| 1G12 Parental | 0.06 | 0.07 | 0.02 | >10 |

### Affinities of Anti-DC-SIGN Antibodies to DC-SIGN

The affinity of various antibodies to DC-SIGN Carbohydrate Recognition Domain (CRD) was determined using Biacore. Purified IgGs for the parental antibodies were titrated to determine Kd values for binding to purified antigen domain by two methods described below.

In method 1 DC-SIGN was used as the ligand (surface attached) and the antibody the analyte (injected at different concentrations). The DC-SIGN CRD was captured via the His tag on a CM5 chip that was prepared by immobilizing 12000RU NeutrAvidin followed by capturing ˜550RU of Tris-NTA biotin. Fresh DC-SIGN was used for each dose. Each cycle consisted of charging the surface with a 120s pulse of 5 mM NiCl₂, capturing the same amount of DC-SIGN, injecting the antibody at the desired concentration, and stripping the Ni²⁺ with pulses of 350 mM EDTA and 500 mM imidazole to remove all DC-SIGN. Antibodies were injected at concentrations between 250 and 31 nM for 180s and allowed to dissociate for 600s. The reverse orientation was used in method 2-antibody the ligand and DC-SIGN the analyte. A CM5 chip was first prepared with mouse anti-human IgG Fc and used to capture the antibodies. Fresh antibody was used for each dose where each cycle consisted of capturing the same amount of antibody (˜100RU), injecting the desired concentration of DC-SIGN, and stripping the surface of all captured antibody with two 30s pulses of 10 mM glycine pH 2.0. DC-SIGN was injected for 180s at concentrations between 500 and 1.95 nM and dissociated for 600s. All experiments were conducted on a GE Biacore 8K at 25° C. with a flow rate of 30 uL/min in 10 mM HEPES, 500 mM NaCl, 2.5 mM Imidazole, 0.05% Tween 20, pH 7.4. Kinetic parameters were calculated using the 8K analysis software.

TABLE 23
Binding of Various Anti-DC-SIGN Antibodies to DC-
SIGN Carbohydrate Recognition Domain by Biacore

| | | high density |
|---|---|---|
| | | DC-SIGN |
| | Ab on chip | CRD on chip |
| | (nM) | (nM) |
| 960 K03 | 955 | 0.8 |
| 906C18 | 1950 | 16 |
| 914M09 | 830 | 10 |
| 956 E02 | 999 | 13 |
| 942K11 | 1260 | 180 |
| 550Ee03 | 523 | 5.8 |
| 952P16 | 395 | 3.8 |
| 952G04 | 346 | 3.7 |
| 958N05 | 174 | 1.7 |
| 892D15 | 47600 | 111 |
| 2b2 | 32 | 0.5 |

### Epitope Binning Using Octet Red96 System

Epitope binning of anti-DC-SIGN parental antibodies was performed using the Octet Red96 system (ForteBio, USA) that measures biolayer interferometry (BLI). For this purpose the DC-SIGN extracellular domain with the AviHis tag (SEQ ID NO: 317) was biotinylated via an AviTag™ utilizing BirA biotin ligase according to Manufacturer's recommendations (Avidity, LLC, USA cat# BirA500). The biotinylated immunogen scaffold was loaded at 0.4 μg/ml onto pre-equilibrated streptavidin sensors (ForteBio, USA). The sensors were then transferred to a solution containing 100 nM antibody A in 1× kinetics buffer (ForteBio, USA). Sensors were briefly washed in 1× kinetics buffer and transferred to a second solution containing 33.3 nM of competitor antibody B. Binding kinetics parameters were determined from raw data using the Octet Red96 system analysis software (Version 6.3, ForteBio, USA). Antibodies were tested in all pairwise combinations, as both Antibody A and as competitor antibody B.

TABLE 24
Antibody Binning Results

| Bin | Antibody |
|---|---|
| 1 | 2B2, 892D15, 960K03, 906C18, 952P16, 942G04 |
| 2 | 914M09, 956E02 |
| 3 | 942K11 |

### Epitope Mapping Using Hydrogen/Deuterium Exchange Mass Spectrometry (HD×MS)

Additional epitope mapping was carried out for antibody 2B2 using HD×MS. DC-SIGN ECD (SEQ ID NO: 319) was concentrated 5× using a 10 kDa MWCO micro-concentrator. 5 μg of protein was used in each sample and DCSIGN ECD/mAb complexes were prepared by mixing an equimolar amount of DC-SIGN ECD (SEQ ID NO: 319) and each mAb separately. Complexes were allowed to form for 30 min. at room temp before labeling.

For non-deuterated, deuterated controls and deuterated complexes, each sample was diluted with the appropriate volume of labeling buffer (50 mM Phosphate buffer, pH 7.6 or pH 8.6, 150 mM NaCl in H₂O) to bring the total volume to 10 μL. Solutions were placed in 1.5 mL vials and placed in a rack at either 0° C. or 20° C. The labeling step for all samples was performed with the addition of 50 μL of labeling buffer (50 mM Phosphate buffer, pH 7.6 or 8.6, 150 mM NaCl in H₂O) to each sample. Solutions were incubated for 5 min. Vials were transferred to an ice water bath and 250 μL of reduction buffer (8M GndHCl, 1M TCEP, pH2.5) was added and mixed. After 2 min, 300 μL of ice cold quench buffer (0.25% formic acid, 12.5% glycerol) was added and the solutions were immediately frozen in liquid nitrogen. Vials were transferred to the −70° C. freezer attached to a PAL autosampler for HDx analysis. Samples were thawed for 2 min and 500 μL was injected through an in-line pepsin column into the LC-MS system. Proteolytic peptides were sequenced by tandem mass spectrometry (MS/MS) and deuteration values were extracted using HDExaminer.

TABLE 25
Antibody 2B2 protected exchange of the amide
hydrogens in the peptides with the sequences

| Peptide | | |
|---|---|---|
| protected | Amino acid sequence | |
| 1 | VVIKSAEEQNF | SEQ ID NO: 320 |
| 2 | LQLQSSRSNRFTWMGLSDL | SEQ ID NO: 321 |
| 3 | NQEGTWQWVDGSPLL | SEQ ID NO: 322 |
| 4 | NQEGTWQWVDGSPLLPSF | SEQ ID NO: 323 |

### Example 6: Preparation of Anti-DC-SIGN-STING Agonist Conjugates

A) Preparation of Anti-DC-SIGN Antibody with Specific Cysteine (Cys) Mutations

Preparation of anti-DC-SIGN antibodies and other antibodies with site-specific cysteine mutations has been described previously in WO 2014/124316 and WO 2015/138615, each of which was incorporated by reference herein.

### Reduction, Reoxidation and Conjugation of Cys Mutant Anti-DC-SIGN Antibodies to STING Agonists

Some compounds described herein comprising a linker were conjugated to Cys residues engineered into an antibody similar to what is described in Junutula J R, et al., Nature Biotechnology 26:925-932 (2008).

Because engineered Cys residues in antibodies expressed in mammalian cells are modified by adducts (disulfides) such as glutathione (GSH) and/or cysteine during biosynthesis (Chen et al. 2009), the modified Cys as initially expressed is unreactive to thiol reactive reagents such as maleimido or bromo-acetamide or iodo-acetamide groups. To conjugate engineered Cys residues, glutathione or cysteine adducts need to be removed by reducing disulfides, which generally entails reducing all disulfides in the expressed antibody. This can be accomplished by first exposing antibody to a reducing agent such as dithiothreitol (DTT) followed by reoxidation of all native disulfide bonds of the antibody to restore and/or stabilize the functional antibody structure. Accordingly, in order to reduce native disulfide bonds and disulfide bonds between the cysteine or GSH adducts of engineered Cys residue(s), freshly prepared DTT was added to previously purified Cys mutant antibodies to a final concentration of 10 mM. After antibody incubation with DTT at 37° C. for 30 minutes, mixtures were buffer exchanged to PBS pH 8.0 by passing through PD-10 columns (GE Healthcare). Alternatively, DTT can be removed by a dialysis step. Samples were incubated at room temperature for up to two days. The reoxidation process was monitored by reverse-phase HPLC, which is able to separate antibody tetramer from individual heavy and light chain molecules. Reactions were analyzed on a PRLP-S 4000A column (50 mm×2.1 mm, Agilent) heated to 80° C. and column elution was carried out by a linear gradient of 30-60% acetonitrile in water containing 0.1% TFA at a flow rate of 1.5 mL/min. The elution of proteins from the column was monitored at 280 nm. Incubation was allowed to continue until reoxidation was complete. After reoxidation, a maleimide-containing compound selected from compound (C1), (C2), (C3), (C4), (C5), (C6), (C7), (C8), (C9), (C10), (C11), (C12), (C13), (C14), (C15), (C16), (C17), (C18), (C19), (C20), (C21), (C22), (C23a), (C23b), (C24), (C25a), (C25b), (C26), (C27), (C28), (C29), (C30), (C31), (C32), (C33), (C34), (C35), (C36a), (C36b), (C37a), (C37b), (C38), (C39), (C40), (C41), (C42a), (C42b), (C43), (C44a), (C44b) or (C45) was added to reoxidized antibody in PBS buffer (pH 7.2) at molar ratios of typically 1:1, 1.5:1, 2.5:1, or 5:1 to engineered Cys, and incubations were carried out for up to 60 minutes at room temperature. Excess free compound was removed by purification over Protein A resin by standard methods followed by buffer exchange into PBS.

Cys mutant antibodies or antibody fragments were alternatively reduced and reoxidized using an on-resin method. Protein A Sepharose beads (1 mL per 10 mg antibody) were equilibrated in PBS (no calcium or magnesium salts) and then added to an antibody sample in batch mode. For Fab samples with a C-terminal His-tag, Ni-NTA resin (Qiagen) was substituted for this step, and the samples were treated similarly to full length antibodies in all other respects. A stock of 0.5 M cysteine was prepared by dissolving 850 mg of cysteine HCl in 10 mL of a solution prepared by adding 3.4 g of NaOH to 250 mL of 0.5 M sodium phosphate pH 8.0 and then 20 mM cysteine was added to the antibody/bead slurry, and mixed gently at room temperature for 30-60 minutes. Beads were loaded to a gravity column and washed with 50 bed volumes of PBS in less than 30 minutes, then the column was capped with beads resuspended in one bed volume of PBS. To modulate the rate of reoxidation, 50 nM to 1 μM copper chloride was optionally added. The reoxidation progress was monitored by removing a small test sample of the resin, eluting in IgG Elution buffer (Thermo), and analyzing by RP-HPLC as described above. Once reoxidation progressed to desired completeness, conjugation could be initiated immediately by addition of 1-5 molar equivalent of compound over engineered cysteines, and allowing the mixture to react for 5-10 minutes at room temperature before the column was washed with at least 20 column volumes of PBS. Antibody conjugates were eluted with IgG elution buffer and neutralized with 0.1 volumes 0.5 M sodium phosphate pH 8.0 and buffer exchanged to PBS. Alternatively, instead of initiating conjugation with antibody on the resin, the column was washed with at least 20 column volumes of PBS, and antibody was eluted with IgG elution buffer and neutralized with buffer pH 8.0. Antibodies were then either used for conjugation reactions or flash frozen for future use.

Anti-DC-SIGN Fab fragments were reduced, re-oxidized, and conjugated using a similar on-resin method. For Fab samples with a C-terminal His-tag, Ni-NTA resin (Qiagen) was substituted for this step, and the samples were treated similarly to full length antibodies for reduction and re-oxidation. As with the full length antibodies, the reduction is used to uncap the native and engineered cysteines (e.g. HC-E152C or HC-E152C-LC-S165C), and the re-oxidation of the native disulfides, including the interchain disulfide, leaves only the introduced cysteines available for combination.

Conjugates were typically buffer exchanged to PBS pH 7.2 and analyzed by methods described below. In some instances, conjugates were further purified by standard preparative size exclusion chromatography methods.

A general reaction scheme for conjugation of the compounds (C1), (C2), (C3), (C4), (C5), (C6), (C7), (C8), (C9), (C10), (C11), (C12), (C13), (C14), (C15), (C16), (C17), (C18), (C19), (C20), (C21), (C22), (C23a), (C23b), (C24), (C25a), (C25b), (C26), (C27), (C28), (C29), (C30), (C31), (C32), (C33), (C34), (C35), (C36a), (C36b), (C37a), (C37b), (C38), (C39), (C40), (C41), (C42a), (C42b), (C43), (C44a), (C44b) or (C45) to an antibody having free thiols (obtained using the methods described above) is given below:

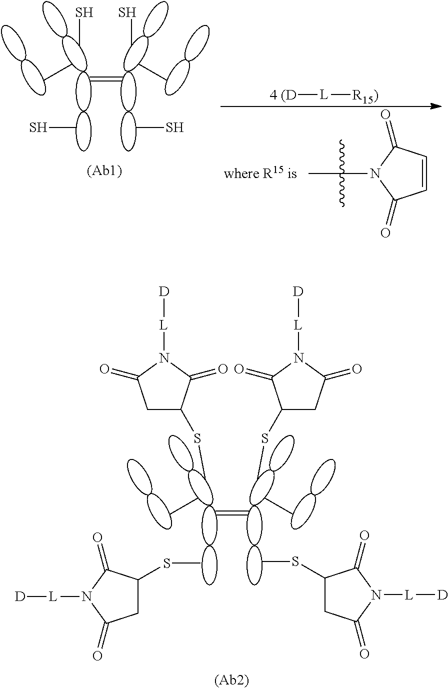

Here, D-L-R₁₅ represents any one of compounds (C1), (C2), (C3), (C4), (C5), (C6), (C7), (C8), (C9), (C10), (C11), (C12), (C13), (C14), (C15), (C16), (C17), (C18), (C19), (C20), (C21), (C22), (C23a), (C23b), (C24), (C25a), (C25b), (C26), (C27), (C28), (C29), (C30), (C31), (C32), (C33), (C34), (C35), (C36a), (C36b), (C37a), (C37b), (C38), (C39), (C40), (C41), (C42a), (C42b), (C43), (C44a), (C44b) or (C45), where D represent the cyclic dinucleotide in each respective compound, L is the linker moiety in each respective compound and R₁₅ is the maleimide group in each respective compound.

### Properties of the Anti-DC-SIGN-STING Agonist Conjugates

Antibody-STING agonist conjugates were analyzed to determine extent of conjugation. A compound-to-antibody ratio was extrapolated from LC-MS data for reduced and deglycosylated samples. LC-MS allows quantitation of the average number of molecules of linker-payload (compound) attached to an antibody in a conjugate sample. HPLC separates antibody into light and heavy chains, and separates heavy chain (HC) and light chain (LC) according to the number of linker-payload groups per chain. Mass spectral data enables identification of the component species in the mixture, e.g., LC, LC+1, LC+2, HC, HC+1, HC+2, etc. From the average loading on the LC and HC chains, the average compound to antibody ratio can be calculated for an antibody conjugate. A compound-to-antibody ratio for a given conjugate sample represents the average number of compound (linker-payload) molecules attached to a tetrameric antibody containing two light chains and two heavy chains.

Conjugates were profiled using analytical size-exclusion chromatography (AnSEC) on Zenix C-300 3 um 7.8×150 mm column (Sepax Technologies). Alternatively, samples were tested on a KW-803 column (TIC Cat#6960940). The purity with respect to aggregation was analyzed based on analytical size exclusion chromatography (AnSEC) and reported as the percent monomer based on AUC of the assigned monomer peak.

Most conjugates achieved high compound-to-antibody ratio and were mainly monomeric. Conjugation through this method results in conjugation efficiencies of greater than 90% for most compounds (Table 26, below). The majority of the conjugates achieve greater than 95% purity as assessed by AnSEC (Table 26). These results suggest that conjugates described herein can be made efficiently and have favorable characteristics.

In the Examples below, unless otherwise indicated, all DC-SIGN conjugates used were the DAR4 version.

TABLE 26
Properties of anti-DC-SIGN-STING agonist conjugates

| | Linker- | | Compound-to- | Conjugation | |
|---|---|---|---|---|---|
| Antibody | Payload | Payload | antibody ratio | Efficiency (%) | % Monomer |
| 2B2 Hz | C1 | T1-1 | 3.9 | 98 | 99 |
| 2B2 Hz | C2 | T1-1 | 3.6 | 90 | ND ^{a} |
| 2B2 Hz | C18 | T1-2 | 4.0 | 100 | ND ^{a} |
| 2B2 Hz | C23 | T1-6 | 3.2 | 80 | ND ^{a} |
| 2B2 Hz | C36 | T1-6 | 3.6 | 90 | ND ^{a} |
| 2B2 Hz | C29 | T1-1 | 3.6 | 90 | ND ^{a} |
| 2B2 Hz | C31 | T1-1 | 3.6 | 90 | ND ^{a} |
| 2B2 Hz DAR2-1 (e152) | C1 | T1-1 | 1.8 | 90 | 99 |
| 2B2 Hz DAR2-2 (s375) | C1 | T1-1 | 2.0 | 100 | 99 |
| 2B2 Hz Fab (DAR1) | C1 | T1-1 | 0.9 | 88 | 99 |
| 2B2 Hz Fab2 (DAR2) | C1 | T1-1 | 1.7 | 83 | 96 |
| 2B2 Chimeric | C1 | T1-1 | 3.8 | 95 | 98 |
| 550 E03 Parental | C1 | T1-1 | 3.7 | 93 | 97 |
| 952P16 Parental | C1 | T1-1 | 3.9 | 97 | 98 |
| 952G04 Parental | C1 | T1-1 | 3.9 | 97 | 99 |
| 958N05 Parental | C1 | T1-1 | 3.8 | 96 | 98 |
| 892D15 Parental | C1 | T1-1 | 3.8 | 96 | 99 |
| 960K03 Parental | C1 | T1-1 | 4.2 | 106 | 96 |
| 906C18 Parental | C1 | T1-1 | 3.9 | 97 | 96 |
| 914M09 Parental | C1 | T1-1 | 4.0 | 100 | 99 |
| 956 E02 Parental | C1 | T1-1 | 3.8 | 95 | 98 |
| 942K11 Parental | C1 | T1-1 | 3.8 | 96 | 98 |
| 958N05 S93A | C31 | T1-1 | 3.9 | 98 | 96 |
| 958N05 S93A | C18 | T1-2 | 3.9 | 97 | 95 |
| 960K03 N92S | C31 | T1-1 | 3.8 | 96 | 96 |
| 960K03 N92S | C18 | T1-2 | TBD ^{b} | TBD ^{b} | 94/99^{c} |
| 1G12 mouse | C1 | T1-1 | 3.5 | 88 | 99 |
| 2B2 Hz | C31 | T1-1 | 4.0 | 100 | 98 |
| 2B2 Hz | C18 | T1-2 | 3.8 | 95 | 94 |
| 2B2 Hz DAR2-1 (e152) | C18 | T1-2 | 1.8 | 90 | 99 |
| 2B2 Hz DAR2-1 (e152) | C31 | T1-1 | 2.0 | 100 | 98 |
| Control DAPA DAR4 | C31 | T1-1 | 4.0 | 100 | 94 |
| Control DAPA DAR4 | C18 | T1-2 | 4.0 | 100 | 94 |

^{a} ND: not determined
^{b} TBD: to be determined
^{c}Values reported before and after preparative SEC.

### Example 7: DC-SIGN Immunoconjugates are Able to Activate Human DCs and Macrophages In Vitro

Primary human monocytes were isolated from a leukapheresis using magnetic bead selection and frozen for storage in liquid nitrogen. For monocyte DC (moDC) differentiation, cells were thawed and incubated in media containing GM-CSF and IL-4 for 7 days. For M2 macrophages (M2 moMacs), cells were thawed and incubated for 6 days with M-CSF containing media and then polarized with the addition of IL-4 for 24 hours. After the differentiation process for both moDC and moMacs, media was washed off and replaced with fresh media containing isotype control (DAPA version of Trastuzumab) C1, or DC-SIGN antibody C1 conjugates. Free T1-1 compound was used as a control. 24 hours after incubation with indicated compounds, cells were evaluated by flow cytometry for activation.

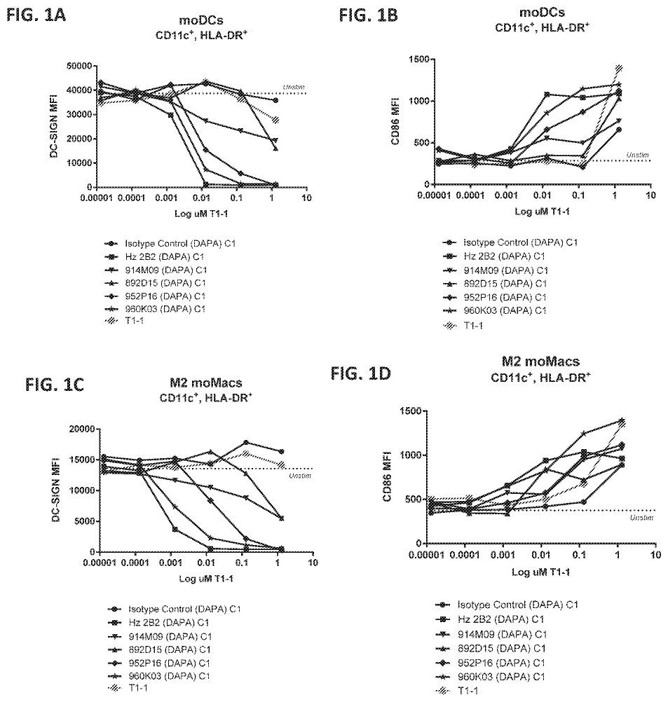

As shown in FIG. 1, all DC-SIGN antibody C1 immunoconjugates induced downregulation of DC-SIGN on monocyte dendritic cells and macrophages, indicating target engagement (FIGS. 1A and 1C). All DC-SIGN antibody C1 immunoconjugates induced monocyte dendritic cell and macrophage activation as measured by CD86 upregulation (FIGS. 1B and 1D).

The differentiated moDC and moMacs were also treated with isotype control (DAPA) or humanized 2B2 (DAPA) conjugated to C1, C18 or C31 payloads. Free T1-1 compound was used as a control. 24 hours after incubation with indicated compounds, cells were evaluated by flow cytometry for activation.

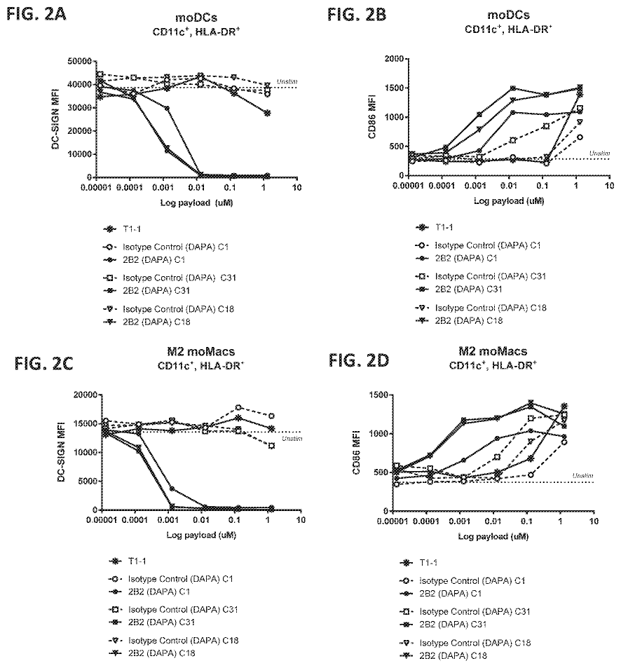

As shown in FIG. 2, 2B2 (DAPA) immunoconjugates of C1, C18 and C31 induced downregulation of DC-SIGN on monocyte dendritic cells and macrophages (FIGS. 2A and 2C), indicating target engagement. 2B2 (DAPA) immunoconjugates of C1, C18 and C31 induced monocyte dendritic cell and macrophage activation as measured by CD86 upregulation (FIGS. 2B and 2D).

DAR2 version of the 2B2 (DAPA) C1 immunoconjugates were tested for activity on human monocyte DCs and macrophages. After the differentiation process, moDC and moMacs were treated with humanized (Hz) 2B2 (DAPA) C1, isotype control (DAPA) C1, Hz 2B2 (DAPA) DAR2 C1, or T1-1. 24 hours after incubation with indicated compounds, cells were evaluated by flow cytometry for activation.

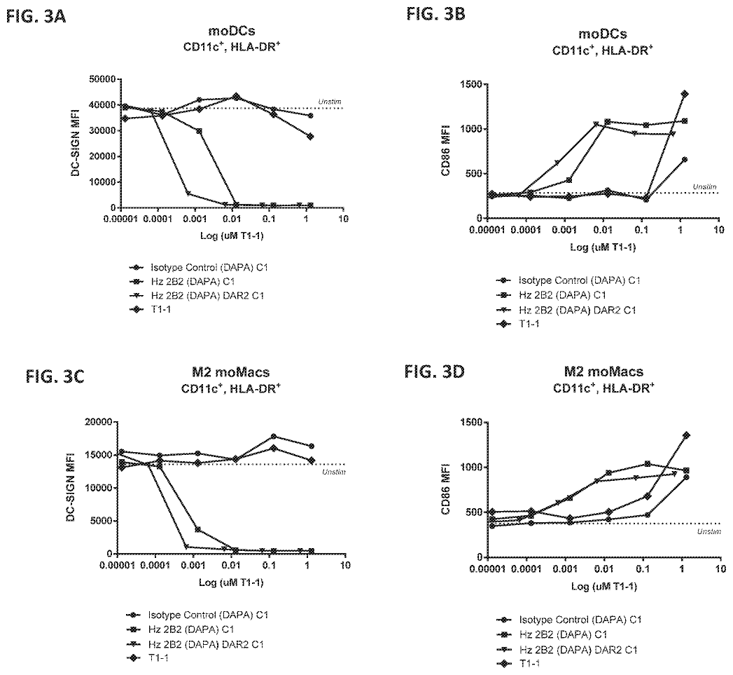

As shown in FIG. 3, Hz 2B2 (DAPA) C1 and Hz 2B2 (DAPA) DAR2 C1 induced downregulation of DC-SIGN on monocyte dendritic cells and macrophages (FIGS. 3A and 3C), indicating target engagement. Hz 2B2 (DAPA) C1 and Hz 2B2 (DAPA) DAR2 C1 induced monocyte dendritic cell and macrophage activation as measured by CD86 upregulation (FIGS. 3B and 3D).

Primary human monocytes were isolated from a leukapheresis using magnetic bead selection and frozen for storage in liquid nitrogen. For monocyte DC (moDC) differentiation, cells were thawed and incubated in media containing GM-CSF and IL-4 for 7 days. After the differentiation process for both moDC and moMacs, media was washed off and replaced with fresh media containing isotype control (DAPA) or 960K03 (DAPA) conjugated to C31 payload. Free T1-1 compound was used as a control. 24 hours after incubation with indicated compounds, cells were evaluated by flow cytometry for activation.

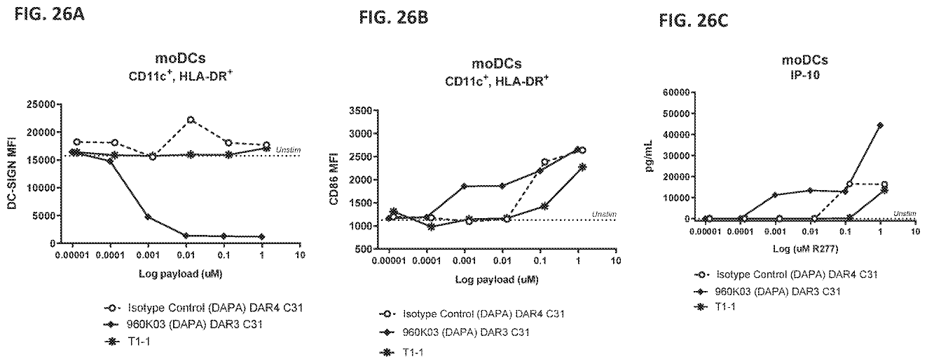

As shown in FIG. 26, 960K03 (DAPA) C31 conjugate induced downregulation of DC-SIGN on monocyte dendritic cells, indicating target engagement (FIG. 26A). 960K03 (DAPA) C31 induced monocyte dendritic cell activation (as measured by CD86 upregulation) with less payload than the isotype control (DAPA) C31 conjugate or unconjugated T1-1 (FIG. 26B). 960K03 (DAPA) C31 also induced IP-10 secretion into the culture supernatant at a higher concentration with less payload than the isotype control (DAPA) C31 conjugate or unconjugated T1-1 (FIG. 26C).

### Example 8: DC-SIGN Immunoconjugates Induce DC Activation and Cytokine Production in Tg+ Mice

Transgenic mice expressing human DC-SIGN gene (Tg+) or DC-SIGN negative control littermates (Tg−) were treated intravenously with 1 mg/kg of Hz 2B2 (DAPA) conjugated to the following payloads: C1, C2, C31, C23a/b, C36a/b or C28. Blood was collected at 6 hours post dose to analyze plasma cytokine and chemokine levels and spleens were analyzed at 24 hours post dose to look at dendritic cell activation.

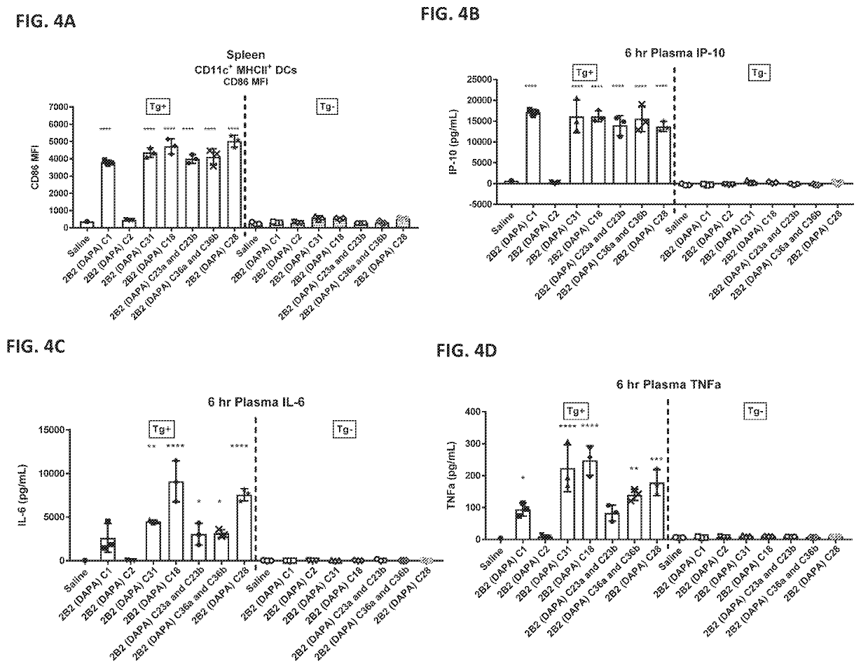

As shown in FIG. 4, in the transgenic mouse model used here, all Hz 2B2 (DAPA) immunoconjugates except for C2 induced proinflammatory cytokine release at 6 hours post dose including IL-6 (FIG. 4C), TNFα (FIG. 4D) and IP-10 (FIG. 4B). All Hz 2B2 (DAPA) immunoconjugates except for C2 induced dendritic cell maturation as measured by CD86 upregulation at 24 hours post dose (FIG. 4A).

Transgenic mice expressing human DC-SIGN gene (Tg+) or transgene-negative littermate control (Tg−) mice were treated with Hz 2B2 (DAPA), 2B2 (DAPA)-C1, or isotype control (DAPA) C1 at 1 milligram per kilogram body weight (mpk) intravenously (i.v.). Mice were bled 6 hours after dosing to collect plasma for analysis of circulating cytokine levels.

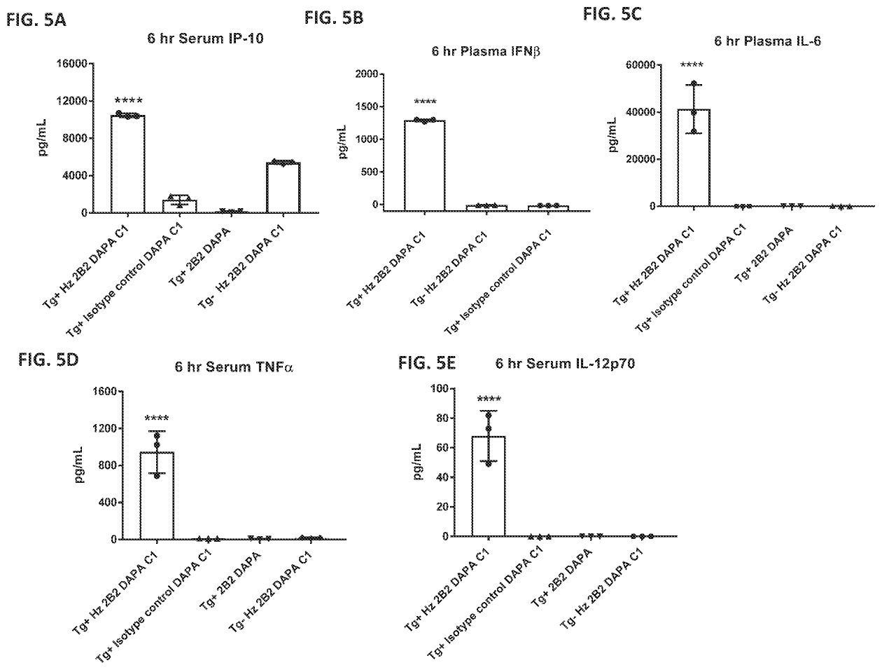

As shown in FIG. 5, Tg+ mice showed a robust increase in circulating plasma IP-10 (FIG. 5A), IFN (FIG. 5B), IL-6 (FIG. 5C), TNFα (FIG. 5D) and IL-12p70 (FIG. 5E).

Spleens were harvested 24 hours post dose and analyzed by flow cytometry to look at CD11c+ dendritic cells.

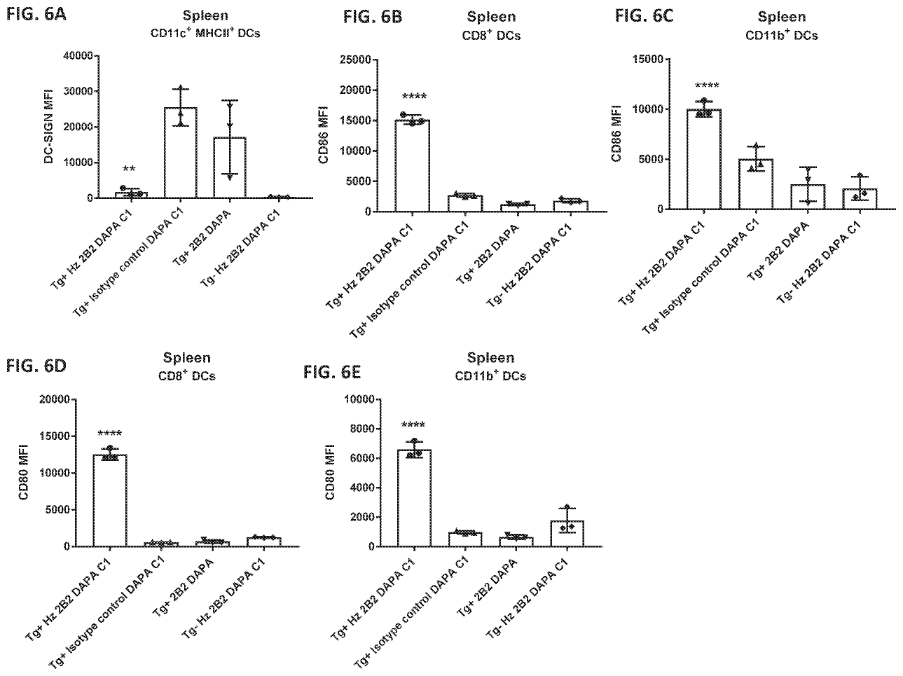

As shown in FIG. 6, DC-SIGN levels were significantly reduced in Tg+ mice treated with humanized 2B2 (DAPA)-C1 (FIG. 6A), indicating target engagement. Both CD80 and CD86 were highly upregulated in CD8+ and CD11b+ DCs from mice treated with humanized 2B2 (DAPA)-C1 (FIGS. 6B-6E), demonstrating dendritic cell activation.

Transgenic mice expressing human DC-SIGN gene (Tg+) or transgene-negative littermate control (Tg−) mice were treated intravenously (i.v.) with 1 mg/kg of the indicated anti-DC-SIGN antibodies (DAPA format) conjugated to C1. Spleens were harvested 24 hours post dose and analyzed by flow cytometry to look at CD11c+ dendritic cells.

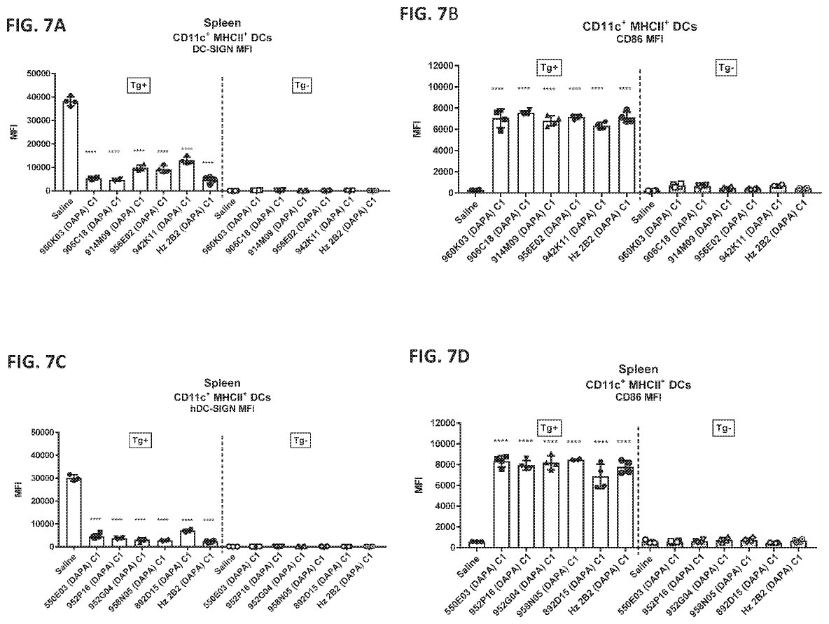

As shown in FIG. 7, Tg+ mice treated with anti-DC-SIGN (DAPA) C1 conjugates had a significant downregulation of surface DC-SIGN (FIGS. 7A and 7C), indicating target engagement. Tg+ mice treated with anti-DC-SIGN (DAPA) C1 conjugates also had a robust upregulation of CD86 on the surface of dendritic cells indicative of DC activation (FIGS. 7B and 7D).

Transgenic mice expressing human DC-SIGN gene (Tg+) or transgene-negative littermate control (Tg−) mice were treated intravenously (i.v.) with 1 mg/kg of the indicated anti-DC-SIGN antibodies (DAPA format) conjugated to C1. Mice were bled 6 hours after dosing to collect plasma for analysis of circulating cytokine levels.

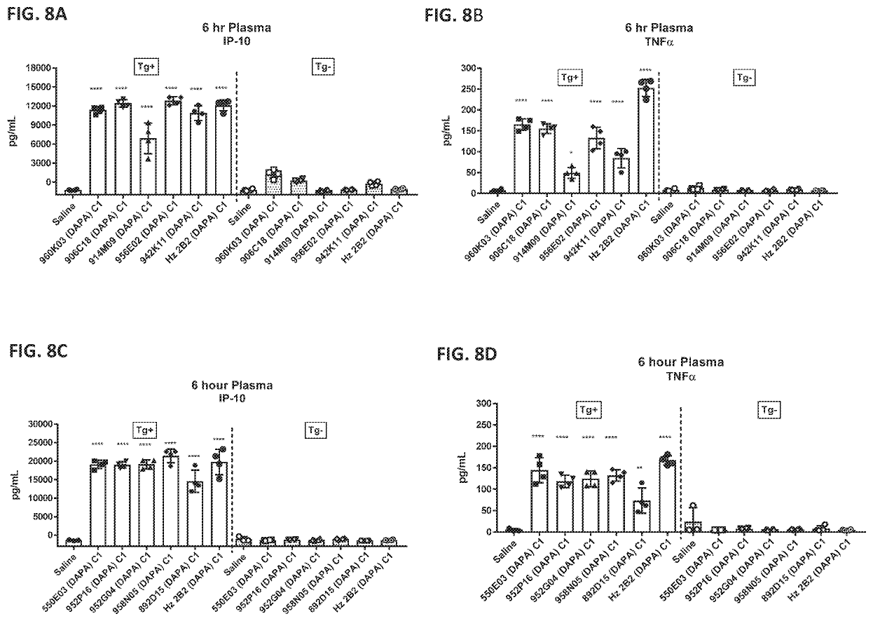

As shown in FIG. 8, Tg+ mice treated with anti-DC-SIGN (DAPA) C1 conjugates showed robust increases in plasma IP-10 (FIGS. 8A and 8C) and TNFα levels (FIGS. 8B and 8D) indicative of activation.

Transgenic mice expressing human DC-SIGN gene (Tg+) or transgene-negative littermate control (Tg−) mice were treated with 960K03 (DAPA) DAR4 C31 at 0.01, 0.03, 0.1, 0.3 or 1 milligram per kilogram body weight (mpk) intravenously (i.v.). Mice were bled 6 hours after dosing to collect plasma for analysis of circulating cytokine levels.

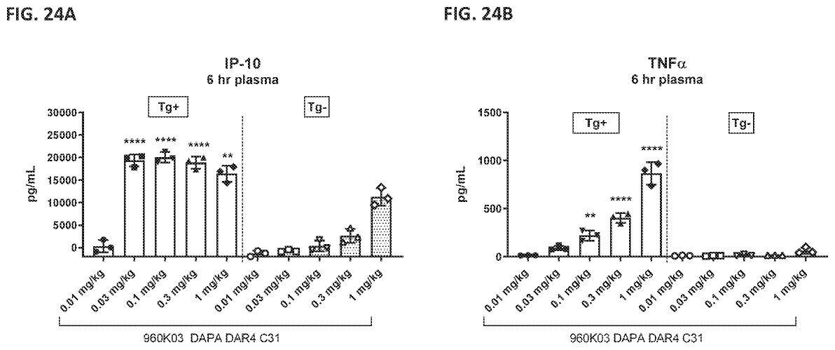

As shown in FIG. 24, Tg+ mice showed a robust increase in circulating plasma IP-10 (FIG. 24A) and TNFα (FIG. 24B).

Transgenic mice expressing human DC-SIGN gene (Tg+) or transgene-negative littermate control (Tg−) mice were treated with 960K03 (DAPA) DAR4 C31 at 0.01, 0.03, 0.1, 0.3 or 1 milligram per kilogram body weight (mpk) intravenously (i.v.). Spleens were harvested 24 hours post dose and analyzed by flow cytometry to look at CD11c+ dendritic cells.

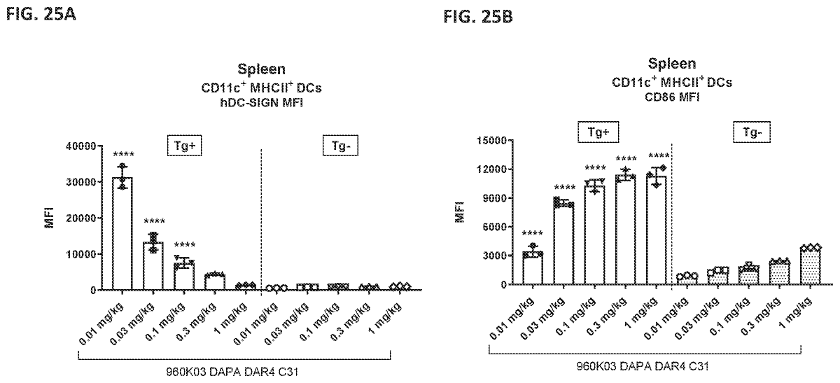

As shown in FIG. 25, DC-SIGN levels were significantly reduced in Tg+ mice treated with 960K03(DAPA) DAR4 C31 (FIG. 25A), indicating target engagement. CD86 was highly upregulated on CD11c+ dendritic cells in a dose dependent manner in Tg+ mice treatment with 960K03(DAPA) DAR4 C31 (FIG. 25B), demonstrating dendritic cell activation

### Example 9: WT, Fc Silent, Fab2 and Fab Versions of 2B2 C1 Immunoconjugates Induce Cytokine Production and DC Activation in Tg+ Mice

Transgenic mice expressing human DC-SIGN gene (Tg+) and Tg− controls were treated intravenously with 1 mg/kg of Hz 2B2 (DAPA) C1, 1 mg/kg of 2B2 C1 (WT Fc), 1.33 mg/kg 2B2 Fab2 DAR2 C1, 1.3 mg/kg 2B2 Fab DAR1 C1 or 1 mg/kg of isotype control (DAPA) C1 conjugates. Blood was collected at 6 hours post dose to analyze plasma IP-10 and IL-12p70 levels. Spleens were analyzed at 24 hours post dose to look at dendritic cell activation.

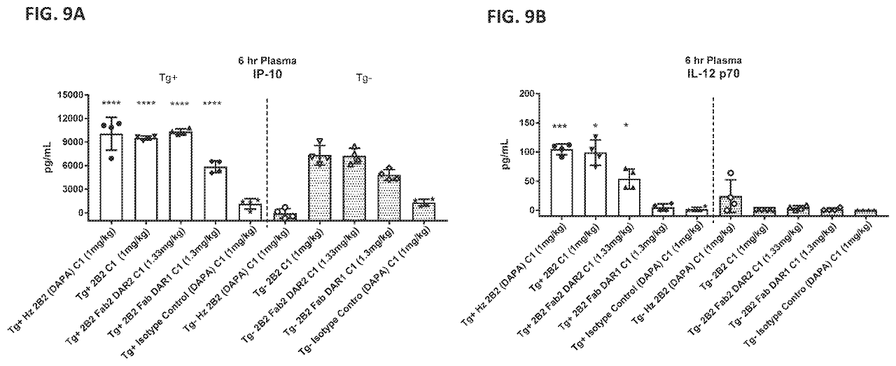

As shown in FIG. 9, DAPA and VVT Fc formats as well as Fab2 and Fab C1 conjugates induced IP-10 production (FIG. 9A). DAPA, VVT and Fab2 formats induced IL-12p70 production in Tg+ mice in a target dependent manner (FIG. 9B).

As shown in FIG. 10, DAPA and VVT Fc formats as well as Fab2 and Fab versions of 2B2 C1 conjugates induced DC-SIGN downregulation (FIG. 10A), indicative of target engagement and CD86 upregulation on DCs (FIG. 10B), indicative of DC activation in Tg+ mice.

The activity on human monocyte derived DCs was tested for the WT and Fc silent formats of the 2B2 C1 immunoconjugate. Primary human monocytes were isolated from a leukapheresis using magnetic bead selection and frozen for storage in liquid nitrogen. For monocyte DC (moDC) differentiation, cells were thawed and incubated in media containing GM-CSF and IL-4 for 7 days. After the differentiation process, media was washed off and replaced with fresh media containing isotype control (DAPA), humanized 2B2 (DAPA), isotype control (WT) or 2B2 (WT) conjugated to C1. Free T1-1 compound was used as a control. 24 hours after incubation with indicated compounds, cells were evaluated by flow cytometry for activation.

As shown in FIG. 11, both WT and DAPA 2B2 C1 conjugates induced downregulation of DC-SIGN on monocyte dendritic cells, indicating target engagement (FIG. 11A). Both VVT and DAPA 2B2 C1 conjugates induced monocyte dendritic cell activation as measured by CD86 upregulation (FIG. 11B).

Transgenic mice expressing human DC-SIGN gene (Tg+) and Tg− controls were treated intravenously with 5 mg/kg of Hz 2B2 (DAPA)-C1 immunoconjugates, 2B2 (Fc silent) C1 immunoconjugates or saline as a control. Blood was collected at 6 hours post dose to analyze plasma IP-10 and TNFα levels.

As shown in FIG. 12, both DAPA and Fc silent versions of 2B2 C1 Immunoconjugates induced high levels of circulating IP-10 (FIG. 12A) and TNFα (FIG. 12B). Spleens were analyzed at 24 hours post dose to look at dendritic cell activation. Both DAPA and Fc silent versions of 2B2 C1 conjugates induced DC-SIGN downregulation (FIG. 12C) indicative of target engagement and CD86 upregulation on DCs (FIG. 12D) indicative of DC activation in Tg+ mice.

### Example 10: DC-SIGN Immunoconjugates Induce Cytokine Production and DC Activation in a Target Dependent Manner

The induction of cytokine production and dendritic cell activation by DC-SIGN immunoconjugates and by free payload was compared. Transgenic mice expressing human DC-SIGN gene (Tg+) were treated intravenously with 1 mg/kg of 2B2 (DAPA) C1 conjugate (approximately equivalent to 0.5 micrograms (μg) of T1-1 compound), 10 μg or 100 μg of free T1-1 compound. Mice were bled 6 hours after dosing and plasma was collected for circulating cytokine analysis.

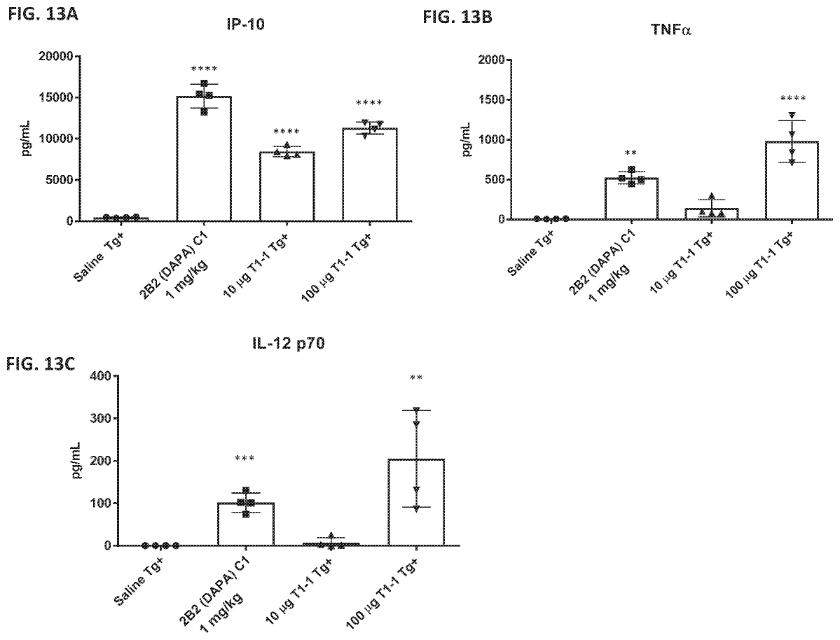

As shown in FIG. 13, Tg+ mice dosed with 1 mg/kg of 2B2 (DAPA) C1 or 100 μg free T1-1 had increased circulating plasma IL-12p70 (FIG. 13C), TNFα (FIG. 13B) and IP-10 (FIG. 13A) levels compared to the untreated Tg+ mice and compared to mice treated with 10 μg of free T1-1 compound.

Transgenic mice expressing human DC-SIGN gene (Tg+) were treated intravenously with 1 mg/kg of 2B2 (DAPA)-C1 immunoconjugates (approximately equivalent to 0.5 micrograms (μg) of T1-1 compound), 10 μg or 100 μg of free T1-1 compound. Mice were sacrificed 24 hours post dosing and spleens were analyzed for CD11c+DC activation by flow cytometry.

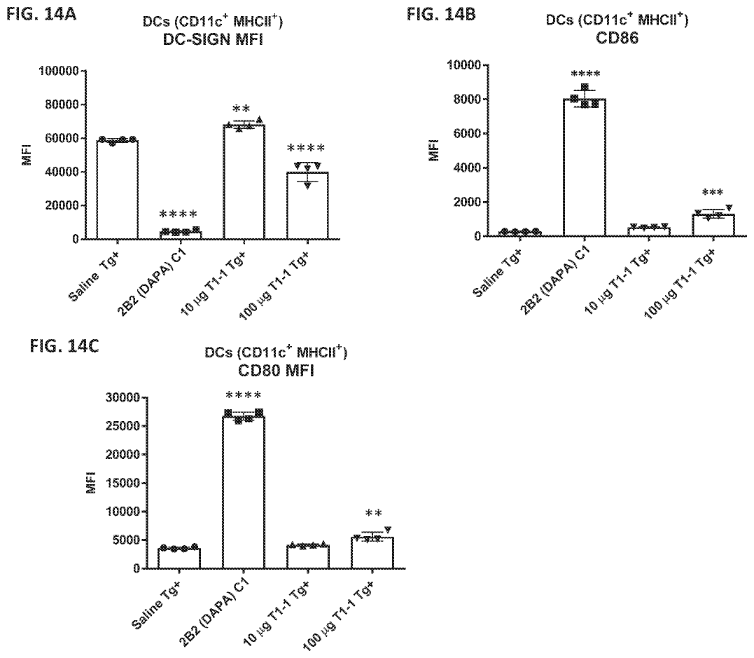

As shown in FIG. 14, DC-SIGN levels were significantly reduced in Tg+ mice treated with humanized 2B2 (DAPA)-C1 (FIG. 14A), indicating target engagement. CD80 and CD86 were significantly upregulated on the surface of DCs from mice treated with 2B2 (DAPA) C1 and to a greater extent than was observed in animals treated with free T1-1 (FIGS. 14B and 14C).

### Example 11: DC-SIGN Immunoconjugates with Different Anti-DC-SIGN Antibodies and in DAR2 Format Induce Cytokine Production and DC Activation

Another DC-SIGN immunoconjugate was evaluated for its activity to induce cytokine production and DC activation. Transgenic mice expressing human DC-SIGN gene (Tg+) or transgene-negative littermate control (Tg−) mice were treated with parental 1G12 (DAPA) C1 (mlgG2a isotype) at 1 milligram per kilogram body weight (mpk) intravenously (i.v.). Mice were bled 6 hours after dosing to collect plasma for analysis of circulating cytokine levels. Spleens were harvested 24 hours post dose and analyzed by flow cytometry to look at CD11c+ dendritic cells.

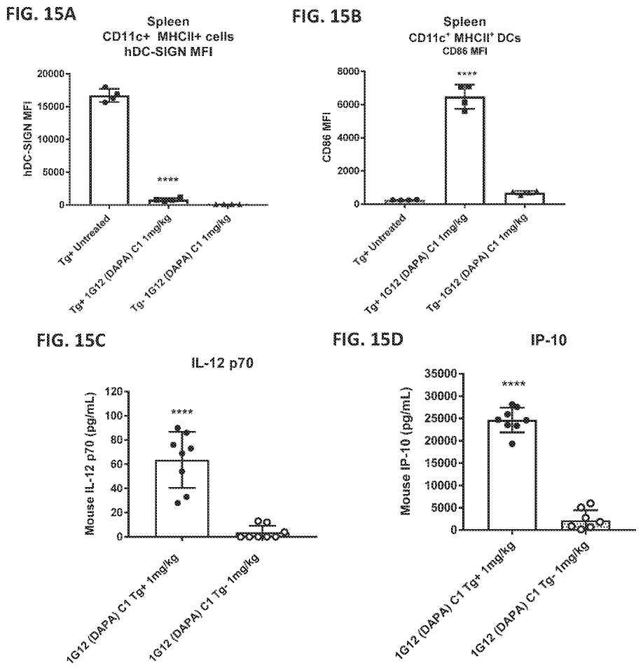

As shown in FIG. 15, Tg+ mice treated with 1G12 (DAPA) C1 had a significant downregulation of surface DC-SIGN (FIG. 15A), indicating target engagement. Tg+ mice treated with 1G12 (DAPA) C1 also had a significant upregulation of CD86 on the surface of dendritic cells indicating activation (FIG. 15B). IP-10 (FIG. 15D) and IL-12p70 (FIG. 15C) plasma levels were significantly increased in Tg+ mice treated with 1G12 (DAPA) C1 at 6 hours post dose, indicative of on target activation through DC-SIGN.

The induction of dendritic cell activation by DAR4 and DAR2 versions of DC-SIGN immunoconjugates was compared. Transgenic mice expressing human DC-SIGN gene (Tg+) were treated intravenously with 1 mg/kg of Hz 2B2 (DAPA) C1 immunoconjugates, 2 mg/kg of Hz 2B2 (DAPA) DAR2 C1 immunoconjugates (dosed to deliver equivalent T1-1 payload as 1 mg/kg dose of 2B2 (DAPA) C1), 1 mg/kg of Hz 2B2 (DAPA) DAR2 C1 immunoconjugates (dosed at the equivalent antibody dose as 1 mg/kg dose of 2B2 (DAPA) C1) or 1 mg/kg of isotype control (DAPA) C1. Blood was collected at 6 hours post dose to analyze plasma IP-10 and IL-12p70 levels and spleens were analyzed at 24 hours post dose to look at dendritic cell activation.

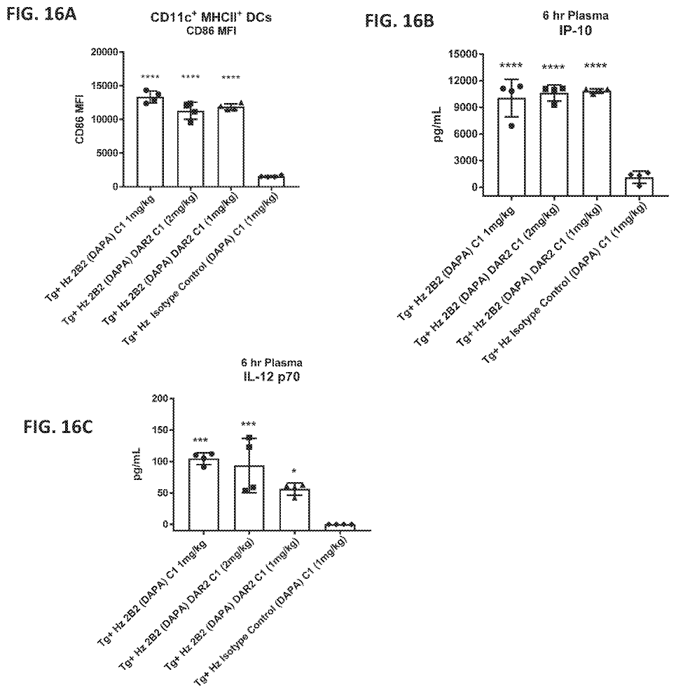

As shown in FIG. 16, both antibody and payload matched doses of 2B2 (DAPA) DAR2-C1 induced DC activation as measured by CD86 upregulation (FIG. 16A) as well as IL-12p70 secretion (FIG. 16C) and IP-10 secretion (FIG. 16B) in a target dependent manner.

### Example 12: DC-SIGN Immunoconjugate Enhances Antibody Responses to DNP-KLH and Promotes Isotype Switching in Tg+ Mice

Transgenic mice expressing human DC-SIGN gene (Tg+) were immunized with DNP-KLH formulated in alum or PBS in alum as a control. One day after immunization, some mice received 1 mg/kg of Hz 2B2 (DAPA) C1 or isotype control (DAPA) C1 intravenously. 10 days post dose, blood plasma was collected and analyzed for DNP binding antibodies by ELISA.

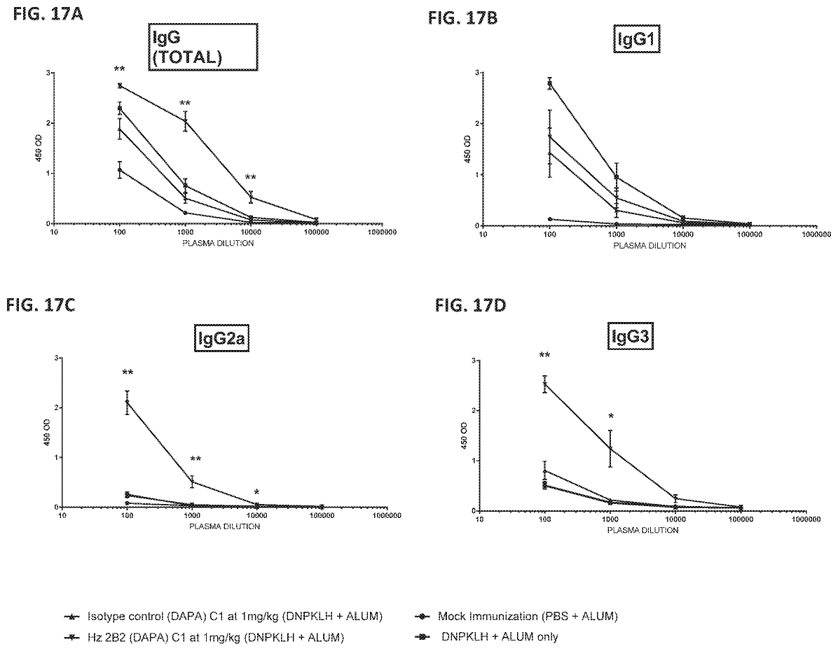

As shown in FIG. 17, mice treated with 2B2 (DAPA) C1 show a significant increase in total DNP binding IgG (FIG. 17A) and also in IgG2a (FIG. 17C) and IgG3 (FIG. 17D) subclasses of DNP binding antibodies but not IgG1 (FIG. 17B).

### Example 13: DC-SIGN Immunoconjugates Delay Tumor Growth in Transgenic Mice Expressing DC-SIGN

Female transgenic mice expressing human DC-SIGN gene (Tg+) or Tg− animals were implanted with 2.5×10⁵ MC38 tumor cells subcutaneously in the hind flank. Tumors were measured 3 times weekly throughout the course of the study. When tumors reached 100-200 cubic millimeters (mm³), mice were treated with a single dose of 1 mg/kg 2B2 (DAPA) or 1 mg/kg 2B2 (DAPA)-C1. Mice were sacrificed 7 days after dosing.

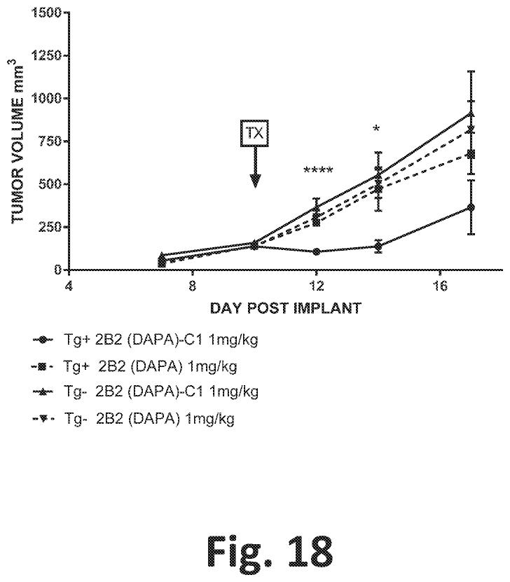

As shown in FIG. 18, DC-SIGN Tg+ mice treated with 1mpk of 2B2 (DAPA) C1 conjugate had significantly delayed tumor growth kinetics, whereas Tg− mice did not show any impairment in tumor growth with either dose of the conjugate.

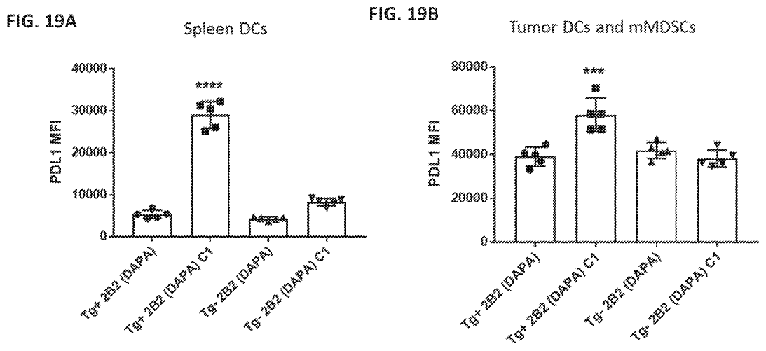

Spleens and tumors were analyzed 24 hours post dose by flow cytometry for PDL1 expression. As shown in FIG. 19, splenic CD11c high dendritic cells (FIG. 19A) and tumor resident dendritic cells and monocytic myeloid derived suppressor cells (mMDSCs) (FIG. 19B) showed a significant upregulation of surface PDL1 in Tg+ mice dosed with 1 mg/kg 2B2 (DAPA) C1.

The effect of DC-SIGN immunoconjugate on tumor T cell infiltration was also evaluated. Female transgenic mice expressing human DC-SIGN gene (Tg+) or Tg− animals were implanted with 2.5×10⁵ MC38 tumor cells subcutaneously in the hind flank. Tumors were measured 3 times weekly throughout the course of the study. When tumors reached 100-200 cubic millimeters (mm³), mice were treated with a single dose of vehicle control (PBS) or 1 mpk 2B2 (DAPA)-C1. Mice After the mice were sacrificed 7 days after dosing, tumors were analyzed for T cell infiltration and activation by flow cytometry.

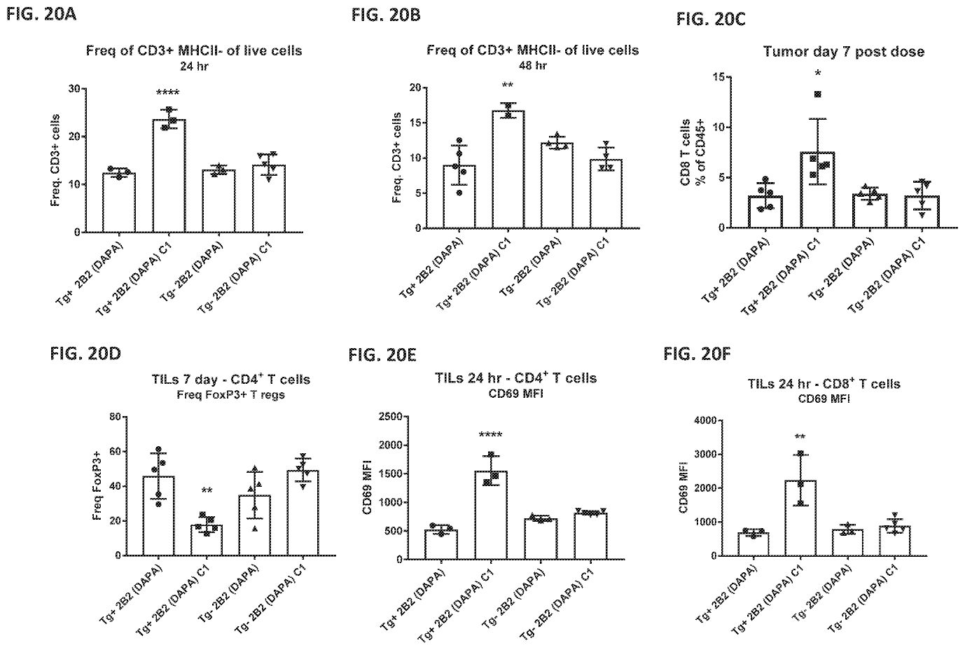

As shown in FIG. 20, increased CD3+ T cells were observed 24 and 48 hours post dosing in Tg+ mice dosed with 2B2 (DAPA) C1 mice (FIGS. 20A and 20B). On day 7 post dose, a significant increase in CD8+ T cells (FIG. 20C) and a significant decrease in FoxP3+T regulatory cells (FIG. 20D) were observed in tumors from Tg+ mice dosed with 2B2 (DAPA) C1. Enhanced T cell activation as measured by CD69 upregulation was seen on CD4 and CD8 T cells in tumors from Tg+ mice dosed with 2B2 (DAPA) C1 24 hours post dose (FIGS. 20E and 20F).

### Example 14: DC-SIGN Immunoconjugate has Enhanced Anti-Tumor Activity in Combination with Anti-PDL1 Therapy

Female transgenic mice expressing human DC-SIGN gene (Tg+) were implanted with 2.5×10⁵ MC38 tumor cells subcutaneously in the hind flank. Tumors were measured 3 times weekly throughout the course of the study. When tumors reached 100-200 cubic millimeters (mm³), mice were treated with a single dose of 1 mg/kg Isotype (DAPA) C1 or 1 mg/kg humanized 2B2 (DAPA) C1. Some groups were given 2 doses of anti-PDL1 clone 10F.9G2 from BioXcell at 10 mg/kg throughout the course of the study (every 3-4 days).

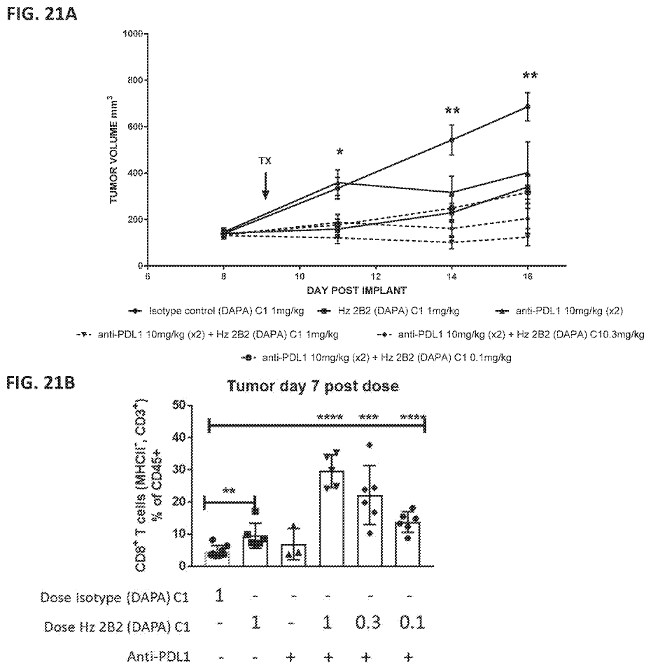

As shown in FIG. 21, mice treated with the combination of 2B2 (DAPA) C1 and anti-PDL1 clone 10F.9G2 showed enhanced reduction in tumor volume (FIG. 21A). 7 days after dosing with 2B2 (DAPA) C1 or Isotype (DAPA) C1, tumors were analyzed by flow cytometry for T cell infiltration. Mice treated with the combination of 2B2 (DAPA) C1 and anti-PDL1 clone 10F.9G2 showed enhanced infiltration of CD8 T cells in their tumors (FIG. 21B).

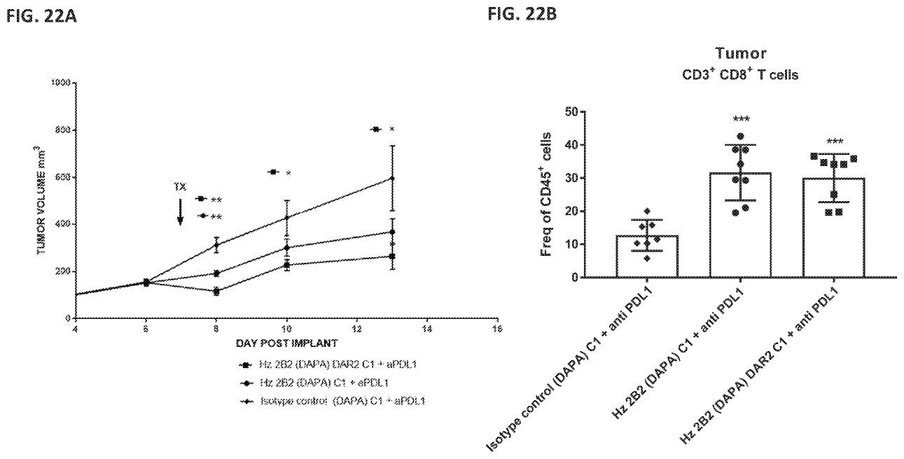

The effect of DAR2 version of DC-SIGN immunoconjugate was also evaluated. As shown in FIG. 22, mice treated with the combination of humanized 2B2 (DAPA) C1 and anti-PDL1 clone 10F.9G2 or humanized 2B2 (DAPA) DAR2 C1 and anti-PDL1 clone 10F.9G2 showed a reduction in tumor volume compared to isotype control treated animals (FIG. 22A). 7 days after dosing with immunoconjugates, tumors were analyzed by flow cytometry for T cell infiltration. Mice treated with the combination of humanized 2B2 (DAPA) C1 or humanized 2B2 (DAPA) DAR2 C1 and anti-PDL1 showed enhanced infiltration of CD8 T cells in their tumors compared to isotype control groups (FIG. 22B).

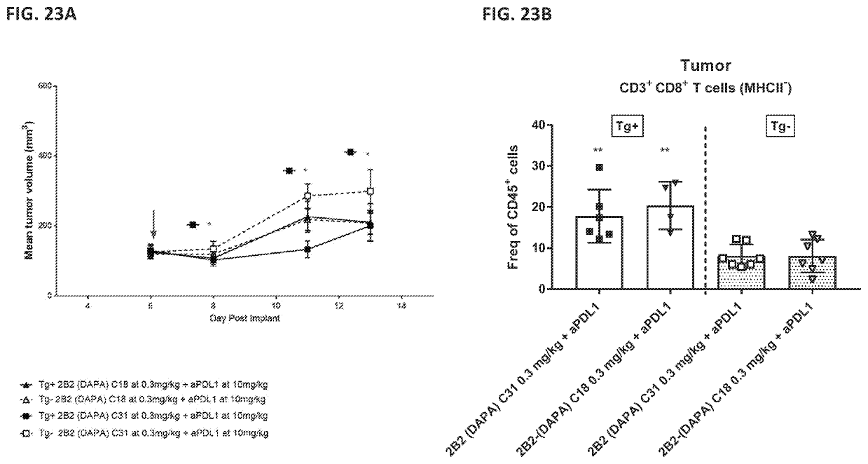

The effect of different payloads of DC-SIGN immunoconjugates were also evaluated. As shown in FIG. 23, Tg+ animals treated with 2B2 (DAPA) C31 in combination with an anti-PDL1 antibody had significantly smaller tumors than Tg− animals (FIG. 23A). Tg+ animals treated with both 2B2 (DAPA) C31 and 2B2 (DAPA) C18 at 0.3 mg/kg in combination with anti-PDL1 had significantly increased tumor CD8+ T cell infiltration compared to Tg− animals treated with the same regimen (FIG. 23B).

Female transgenic mice expressing human DC-SIGN gene (Tg+) or DC-SIGN negative littermate controls (Tg−) were implanted with 2.5×10⁵ MC38 tumor cells subcutaneously in the hind flank. Tumors were measured 3 times weekly throughout the course of the study. When tumors reached 100-200 cubic millimeters (mm³), mice were given a single treatment of 0.1, 0.3 or 1 mg/kg 960K03 (DAPA) DAR4 C31. A control group received no 960K03 (DAPA) DAR4 C31. All groups were given 2 doses of anti-PDL1 clone 10F.9G2 at 10 mg/kg throughout the course of the study (every 3-4 days). 7 days after dosing with 960K03 (DAPA) DAR4 C31, tumors were analyzed by flow cytometry for T cell infiltration.

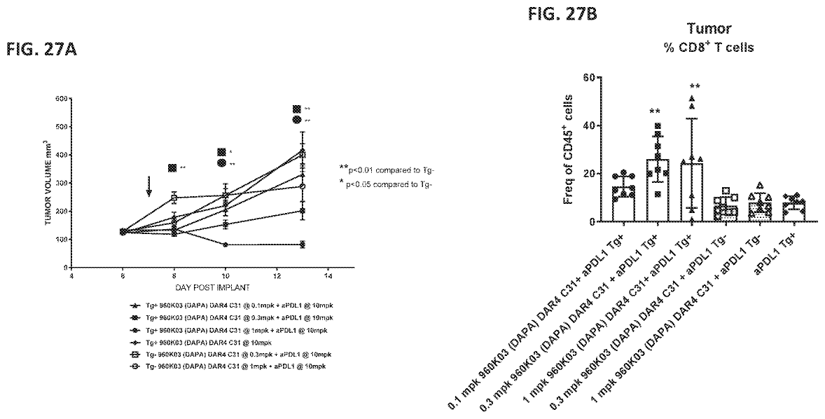

As shown in FIG. 27, mice treated with the combination of 960K03 (DAPA) DAR4 C31 and anti-PDL1 showed enhanced reduction in tumor volume at both 0.3 mg/kg as well as the 1 mg/kg dose levels of 960K03 (DAPA) DAR4 C31 (FIG. 27A). Mice treated with the 960K03 (DAPA) DAR4 C31 and anti-PDL1 showed enhanced infiltration of CD8+ T cells in their tumors when compared to dose matched Tg− controls (FIG. 27B).

### Materials and Methods Used in Examples

### Mouse Tumor Experiments and Drug Antibody Conjugate Treatment.

MC38 cells were grown in 10% Dulbecco's Modified Eagle Medium (DMEM) at 80% confluence prior to implant. Cells growing in log phase were harvested and washed with Hank's Balanced Salt Solution (HBSS) prior to implant. 100 ul of 2.5×10e6 MC38 cells were implants subcutaneously in the hind flank of mice, using insulin syringes, gauge 31. Mice were anesthetized with isoflurane, shaved prior to implant and measured for body weight. Starting at day 5-7 post implant mice were measured using digital calipers using the formula V=(W(2)×L)/2 to determine tumor volume in mm³ (W=tumor width, L=tumor length). Mice were measured every other day and monitored for signs of distress, body weight loss and possible ulcerations. Compounds were administered intravenously when tumors were between 100-200 mm³ using a 1 ml syringe with a 27½ gauge needle. Retro-orbital intravenous injection of immunoconjugates (200 μl) and/or checkpoint blockade was administered under anesthesia. Unless otherwise stated, drug-antibody conjugate dosing was once and anti-PDL1 treatment was 2-3 times throughout the study with 3-4 days in between doses. Anti-PDL1 clone 10F.9G2 was purchased from BioXCell and used at 10 mg/kg where indicated. Where indicated, blood was collected at 6 and 24 hours post dose. Mice were sacrificed at indicated time points post dose and tumors, spleen and lymph nodes were harvested for analysis

### DNP-KLH Immunization

Mice were anesthetized and shaved along the hind flank, and measured for baseline body weight. Day 0, mice were injected with either Phosphate Buffered Saline (PBS) in alum (Serve) or 100 μg of DNP-KLH (Calbiochem) in alum (Serve) (see preparation instructions below) intraperitoneally, 100 μl total volume. 24 hours later mice were given an intravenous dose (200p1) of either isotype control or DCSIGN antibody drug conjugate retro-orbitally under anesthesia. Mice were measured for body weight loss throughout the study. 10 days post immunization with DNP-KLH/alum mice were bled, and spleens removed for analysis. Blood was spun at 5000 rpm for 5 minutes, plasma was harvested and frozen at −20° C. until analysis by ELISA. Spleens were analyzed by flow cytometry.

### DNP ELISA

0.05 mg/mL DNP-BSA (Thermo Fisher) in carbonate buffer was used to coat Nunc ELISA plates. Plates were washed with PBS Tween buffer and blocked with BSA in PBS. Plasma from animals were added in serial dilution and was tested at 1/1000, 1/10000, 1/100000, 1/1000000 dilutions. Plates were washed and secondary antibody was added as indicated (Goat anti-mouse IgG1-HRP, Goat anti-mouse IgG2a-HRP, Goat anti-mouse IgG3-HRP or Goat anti-mouse total H+L chain IgG-HRP). After washing, plates were developed with TMB substrate and the reaction was stopped after 5-30 minutes with the addition of 1N HCl. OD was determined at 450 nM using a plate reader.

### Tumor and Spleen Processing and Flow Cytometry Protocol:

Tumors and/or spleens were extracted at the timepoints indicated from animals. Spleens were processed into a single cell suspension using glass slides and passed through a 100 micron mesh filter. Spleens were lysed in 1 mL of ACK lysis buffer (Life Technologies) for 5 minutes at room temperature. After lysis, cells were pelleted and resuspended in complete RMPI medium (RPMI Media 1640 with 10 percent fetal bovine serum (FBS), 0.05 mM 2-mercaptoethanol, 1 percent Penicillin-Streptomycin-Glutamine, 1 percent non-essential amino acids, 1 percent HEPES, 1 percent sodium pyruvate (all media reagents from Thermo Fisher). Tumors were extracted and put into digestion media in gentleMACS C tubes. Digestion media consists of Dulbecco's Modified Eagle Medium with 0.04 U/mL Dispase (StemCell Technologies), 0.1 mg/mL Collagenase P (Sigma) and 0.1 mg/mL DNase (Sigma). Tumors were incubated with in digestion media and then processed using the gentleMACS Dissociator (Miltenyi Biotec Inc, San Diego, Calif.) to obtain a single cell suspension. After processing, cells were filtered in 100 uM filters (Miltenyi Biotec Inc).

1-2 million cells for each sample were then stained with a cocktail of antibodies to determine impact of the treatments on dendritic cells, myeloid cells and T cells. For FACS analysis, cells were stained with a fixable, amine reactive dye to label dead cells (Zombie UV™ fixable viability kit, Biolegend) in PBS. For antibody staining, indicated antibodies (see table below) were diluted in PBS with 0.5% Bovine serum albumin (BSA, from Sigma). Samples were incubated at 4° C. for 30 minutes and then washed 2 times with PBS with 0.5% BSA. Cells were fixed with stabilizing fixative (BD). For intracellular analysis of FoxP3 to evaluate T regulatory cells, cells were fixed and permeabilized with the FoxP3 transcription factor kit according to manufacturer's recommendations (Thermo Fisher). Cells were then stained with FoxP3 clone FJK-16s (Thermo Fisher). After staining, cells were evaluated on the BD LSRFortessa™ cell analyzer (BD Biosciences, San Jose, Calif.).

T cells were identified as CD3+ MHCII− cells. CD8+ T cells and CD4+ T cells were further defined as CD8 and CD4 positive, respectively. Tregs were identified from CD4+ T cells as being FoxP3+.

Dendritic cells were identified as MHCII high CD11c high cells and further gated on expression of CD8 and CD11 b to identify CD8+DC subsets and CD11b+ DCs where noted. Monocytic myeloid derived suppressor cells were identified as CD45+ cells in tumors that express CD11b, MHCII, F4/80, Ly6C and are intermediate for Ly6G.

TABLE 27
FACS antibodies

| | | | Species |
|---|---|---|---|
| Marker | Clone | Vendor | Reactvity |
| CD45 | 30F11 | BD | Mouse |
| CD8 | 53-6.7 | BD | Mouse |
| Ly6G | 1A8 | Biolegend | Mouse |
| CD11b | M1/70 | Biolegend | Mouse |
| CD11c | N418 | Biolegend | Mouse |
| CD86 | GL-1 | Biolegend | Mouse |
| PDL1 | 10F.9G2 | Biolegend | Mouse |
| Ly6C | HK1.4 | Biolegend | Mouse |
| MHCII | M5.114 | Biolegend | Mouse |
| CD4 | GK1.5 | Biolegend | Mouse |
| CD44 | IM-7 | Biolegend | Mouse |
| CD69 | H1.2F3 | Biolegend | Mouse |
| CD62L | MEL-14 | Biolegend | Mouse |
| PD-1 | J43 | eBioscience/ | Mouse |
| | | Thermo Fisher | |
| F4/80 | BM8 | Biolegend | Mouse |
| CD3 | 17A2 | Biolegend | Mouse |
| HLA-DR | L243 | Biolegend | Human |
| CD86 | IT2.2 | Biolegend | Human |
| CD11c | 3.9 | Biolegend | Human |
| DC-SIGN | 9E9A8 | Biolegend | Human |

### Monocyte Isolation

Peripheral blood Leukopaks from normal human donors were obtained from HemaCare. Leukopaks were aliqouted into 50 mL conical tubes (BD Falcon) and centrifuged at 300 g to 30 minutes to pellet cells. Cells were resuspended in Phosphate Buffered Saline (PBS) containing 2% FBS and 1 mM EDTA to a final concentration of 10⁸ per mL. EasySep Human CD14 Positive Selection Cocktail (StemCell Technologies) was added at 100 μL per mL of cells. CD14+ cells were obtained by positive magnetic selection by following manufactures recommended protocol. Following selection cells were pelleted by centrifugation at 300 g for 10 minutes and resuspended in Recovery™ Cell Culture freezing medium (Thermo Fisher) at 50-100 million cells per mL in cryovials. Cells were frozen in −80 degree C. freezer for at least one day and transferred to liquid nitrogen for storage. Cells were kept in liquid nitrogen until use.

moDC and M2 Macrophage Differentiation

Human CD14+ monocytes were isolated and frozen as described. On the day of differentiation, previously collected and frozen CD14+ monocytes were thawed in a 37 degree C. water bath until just thawed and added immediately to prewarmed complete RPMI medium (cRPMI). Cells were then spun at 1500 rotations per minute (rpm) for 5 minutes in a table top centrifuge to pellet cells. Medium was removed and cells were resuspended in fresh, prewarmed cRPMI medium. Cells were counted and plated at 40,000-80,000 cells per well in a 384 well flat bottom tissue culture plate (Greiner).

For monocyte dendritic cell (moDC) differentiation, cells were cultured in 40 μL final volume with 53 ng/mL of recombinant human GM-CSF (R4D Systems) and 20 ng/mL recombinant human IL-4 (R&D Systems) for 7 days. Cells were washed and fresh, cRPMI was added prior to stimulation with compounds or antibody drug conjugates.

For M2 macrophage differentiation, cells were cultured in 40 μL final volume with a final concentration of 100 ng/mL of recombinant human MCSF. 6 days after differentiation, 20 ng/mL of IL-4 was added to polarize macrophages to an M2 phenotype. 24 hours after polarization, cells were washed and fresh, cRPMI was added prior to stimulation with compounds or antibody drug conjugates.

24 hours after activation with compounds, cells were evaluated by flow cytometry according to the described protocol using antibody clones described in flow cytometry protocol section. DC-SIGN+CD11c+ HLA-DR+ cells were identified and assessed for CD86 expression and levels of DC-SIGN.

### IP-10 ELISA

Plasma was collected at indicated timepoints and analyzed with a Mouse IP-10 Platinum ELISA kit (eBioscience Affymetrix). Plasma was diluted 1:100 and the protocol was followed according to the manufacturer's recommendations. Data was collected using an Enspire spectro-photometer using 450 nM as the primary wavelength.

### IFNβ ELISA

Plasma was collected at indicated timepoints and analyzed with a Mouse IFN-beta ELISA kit (R&D Systems) according to the manufacturer's recommendations. Data was collected using an Enspire spectro-photometer using 450 nM as the primary wavelength.

### MesoScale Discovery Cytokine Analysis (MSD)

Plasma was collected at indicated timepoints and analyzed with a mouse Proinflammatory Panel 1 (mouse) Kit V-PLEX™ 10 plex from MesoScale Discovery. 25 μL of plasma per sample was used and protocol was followed according to the manufacturer's recommendations. Data were collected and analyzed using a Sector Imager 6000.

### Mouse Info and Breeding

Human DC-SIGN transgenic mice (Tg+) (Schaefer et al., J. Immunol. (2008) 180 (10) 6836-6845) were bred to Signr1 deficient mice (−/− or KO) (Orr et al., Glycobiology (2013) 23(3): 363-380). Human DC-SIGN expression was checked using PCR to genotype the mice. Human DC-SIGN Tg+ Signr1−/− mice or human DC-SIGN Tg− Signr1−/− mice were tested with compounds as indicated in the above examples.

Unless defined otherwise, the technical and scientific terms used herein have the same meaning as they usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. An immunoconjugate comprising an anti-DC-SIGN antibody (Ab), or a functional fragment thereof, coupled to an agonist of Stimulator of Interferon Genes (STING) receptor (D) via a linker (L), wherein the linker optionally comprises one or more cleavage elements.

2. The immunoconjugate of claim 1 comprising Formula (I):
Ab-(L-(D)ₘ)ₙ (Formula (I))
wherein:
Ab is an anti-DC-SIGN antibody or a functional fragment thereof;
L is a linker comprising one or more cleavage elements;
D is a drug moiety that has agonist activity against STING receptor;
m is an integer from 1 to 8; and
n is an integer from 1 to 20.

3. The immunoconjugate of claim 1 comprising Formula (I):
Ab-(L-(D)ₘ)ₙ (Formula (I))
wherein:
Ab is an anti-DC-SIGN antibody or a functional fragment thereof;
L is a linker, optionally wherein the linker comprises one or more cleavable elements;
D is a drug moiety that binds to STING receptor or has agonist activity against STING receptor;
m is an integer from 1 to 8; and
n is an integer from 1 to 20;
wherein D, or a cleavage product thereof, that is released from the immunoconjugate has STING agonist activity; or
wherein the immunoconjugate delivers D, or a cleavage product thereof, to a cell targeted by the Ab, and wherein D, or the cleavage product thereof, has STING agonist activity.

4-6. (canceled)

7. The immunoconjugate of claim 1 for delivery of a STING receptor agonist to a cell, the immunoconjugate comprising Formula (I):
Ab-(L-(D)ₘ)ₙ (Formula (I))
wherein:
Ab is an anti-DC-SIGN antibody or a functional fragment thereof;
L is a linker comprising one or more cleavage elements;
D is a drug moiety that binds to STING receptor;
m is an integer from 1 to 8; and
n is an integer from 1 to 20;
wherein the immunoconjugate specifically binds to DC-SIGN on the cell surface and is internalized into the cell, and wherein D, or a cleavage product thereof, is cleaved from L and has STING agonist activity as determined by one or more STING agonist assays selected from: an interferon stimulation assay, a hSTING wt assay, a THP1-Dual assay, a TANK binding kinase 1 (TBK1) assay, or an interferon-γ-inducible protein 10 (IP-10) secretion assay.

8. The immunoconjugate of any one of the preceding claims, wherein D, or the cleavage product thereof, has STING agonist activity if it binds to STING and is able to stimulate production of one or more STING-dependent cytokines in a STING-expressing cell at least 1.1-fold, 1.2-fold, 1.3-fold, 1.4-fold, 1.5-fold, 1.6-fold, 1.7-fold, 1.8-fold, 1.9-fold, 2-fold or greater than an untreated STING-expressing cell.

9. The immunoconjugate of claim 8, wherein the STING-dependent cytokine is selected from interferon, type 1 interferon, IFN-α, IFN-β, type 3 interferon, IFNλ, IP10, TNF, IL-6, CXCL9, CCL4, CXCL11, CCL5, CCL3, or CCL8.

10-18. (canceled)

19. The immunoconjugate of claim 1, wherein the Ab specifically binds to human DC-SIGN.

20. The immunoconjugate of claim 19, wherein the Ab does not bind to human L-SIGN.

21-22. (canceled)

23. The immunoconjugate of claim 1, wherein the Ab comprises a modified Fc region.

24. The immunconjugate of claim 23, wherein the Ab comprises cysteine at one or more of the following positions, which are numbered according to EU numbering:
(a) positions 152, 360 and 375 of the antibody heavy chain, and
(b) positions 107, 159, and 165 of the antibody light chain.

25. (canceled)

26. The immunoconjugate of claim 1, wherein the anti-DC-SIGN antibody comprises:
a. a heavy chain variable region that comprises an HCDR1 (Heavy Chain Complementarity Determining Region 1) of SEQ ID NO:1, an HCDR2 (Heavy Chain Complementarity Determining Region 2) of SEQ ID NO:2, and an HCDR3 (Heavy Chain Complementarity Determining Region 3) of SEQ ID NO:3; and a light chain variable region that comprises an LCDR1 (Light Chain Complementarity Determining Region 1) of SEQ ID NO:14, an LCDR2 (Light Chain Complementarity Determining Region 2) of SEQ ID NO:15, and an LCDR3 (Light Chain Complementarity Determining Region 3) of SEQ ID NO:16;
b. a heavy chain variable region that comprises an HCDR1 of SEQ ID NO:25, an HCDR2 of SEQ ID NO:26, and an HCDR3 of SEQ ID NO:27; and a light chain variable region that comprises an LCDR1 of SEQ ID NO:38, an LCDR2 of SEQ ID NO:39, and an LCDR3 of SEQ ID NO:40;
c. a heavy chain variable region that comprises an HCDR1 of SEQ ID NO:49, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:60;
d. a heavy chain variable region that comprises an HCDR1 of SEQ ID NO:74, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:82;
e. a heavy chain variable region that comprises an HCDR1 of SEQ ID NO:88, an HCDR2 of SEQ ID NO:26, and an HCDR3 of SEQ ID NO:50; and a light chain variable region that comprises an LCDR1 of SEQ ID NO:94, an LCDR2 of SEQ ID NO:95, and an LCDR3 of SEQ ID NO:82;
f. a heavy chain variable region that comprises an HCDR1 of SEQ ID NO:111, an HCDR2 of SEQ ID NO:26, and an HCDR3 of SEQ ID NO:27; and a light chain variable region that comprises an LCDR1 of SEQ ID NO:38, an LCDR2 of SEQ ID NO:39, and an LCDR3 of SEQ ID NO:118;
g. a heavy chain variable region that comprises an HCDR1 of SEQ ID NO:49, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:124;
h. a heavy chain variable region that comprises an HCDR1 of SEQ ID NO:74, an HCDR2 of SEQ ID NO:26, and an HCDR3 of SEQ ID NO:50; and a light chain variable region that comprises an LCDR1 of SEQ ID NO:59, an LCDR2 of SEQ ID NO:39, and an LCDR3 of SEQ ID NO:124;
i. a heavy chain variable region that comprises an HCDR1 of SEQ ID NO:88, an HCDR2 of SEQ ID NO:26, and an HCDR3 of SEQ ID NO:50; and a light chain variable region that comprises an LCDR1 of SEQ ID NO:94, an LCDR2 of SEQ ID NO:95, and an LCDR3 of SEQ ID NO:124;
j. a heavy chain variable region that comprises an HCDR1 of SEQ ID NO:138, an HCDR2 of SEQ ID NO:139, and an HCDR3 of SEQ ID NO:140; and a light chain variable region that comprises an LCDR1 of SEQ ID NO:59, an LCDR2 of SEQ ID NO:39, and an LCDR3 of SEQ ID NO:118;
k. a heavy chain variable region that comprises an HCDR1 of SEQ ID NO:153, an HCDR2 of SEQ ID NO:154, and an HCDR3 of SEQ ID NO:155; and a light chain variable region that comprises an LCDR1 of SEQ ID NO:166, an LCDR2 of SEQ ID NO:167, and an LCDR3 of SEQ ID NO:168;
l. a heavy chain variable region that comprises an HCDR1 of SEQ ID NO:178, an HCDR2 of SEQ ID NO:179, and an HCDR3 of SEQ ID NO:180; and a light chain variable region that comprises an LCDR1 of SEQ ID NO:191, an LCDR2 of SEQ ID NO:192, and an LCDR3 of SEQ ID NO:193;
m. a heavy chain variable region that comprises an HCDR1 of SEQ ID NO:203, an HCDR2 of SEQ ID NO:204, and an HCDR3 of SEQ ID NO:205; and a light chain variable region that comprises an LCDR1 of SEQ ID NO:216, an LCDR2 of SEQ ID NO:217, and an LCDR3 of SEQ ID NO:218;
n. a heavy chain variable region that comprises an HCDR1 of SEQ ID NO:227, an HCDR2 of SEQ ID NO:228, and an HCDR3 of SEQ ID NO:229; and a light chain variable region that comprises an LCDR1 of SEQ ID NO:216, an LCDR2 of SEQ ID NO:217, and an LCDR3 of SEQ ID NO:238;
o. a heavy chain variable region that comprises an HCDR1 of SEQ ID NO:244, an HCDR2 of SEQ ID NO:26, and an HCDR3 of SEQ ID NO:245; and a light chain variable region that comprises an LCDR1 of SEQ ID NO:253, an LCDR2 of SEQ ID NO:254, and an LCDR3 of SEQ ID NO:255;
p. a heavy chain variable region that comprises an HCDR1 of SEQ ID NO:264, an HCDR2 of SEQ ID NO:265, and an HCDR3 of SEQ ID NO:266; and a light chain variable region that comprises an LCDR1 of SEQ ID NO:277, an LCDR2 of SEQ ID NO:278, and an LCDR3 of SEQ ID NO:279;
q. a heavy chain variable region that comprises an HCDR1 of SEQ ID NO:264, an HCDR2 of SEQ ID NO:265, and an HCDR3 of SEQ ID NO:296; and a light chain variable region that comprises an LCDR1 of SEQ ID NO:277, an LCDR2 of SEQ ID NO:278, and an LCDR3 of SEQ ID NO:279.

27. The immunoconjugate of claim 1, wherein the anti-DC-SIGN antibody comprises:
a. A heavy chain variable region (VH) comprising the amino acid sequence of SEQ ID NO:10, and a light chain variable region (VL) comprising the amino acid sequence of SEQ ID NO:21;
b. A heavy chain variable region (VH) comprising the amino acid sequence of SEQ ID NO:34, and a light chain variable region (VL) comprising the amino acid sequence of SEQ ID NO:45;
c. A heavy chain variable region (VH) comprising the amino acid sequence of SEQ ID NO:55, and a light chain variable region (VL) comprising the amino acid sequence of SEQ ID NO:64;
d. A heavy chain variable region (VH) comprising the amino acid sequence of SEQ ID NO:34, and a light chain variable region (VL) comprising the amino acid sequence of SEQ ID NO:70;
e. A heavy chain variable region (VH) comprising the amino acid sequence of SEQ ID NO:78, and a light chain variable region (VL) comprising the amino acid sequence of SEQ ID NO:84;
f. A heavy chain variable region (VH) comprising the amino acid sequence of SEQ ID NO:90, and a light chain variable region (VL) comprising the amino acid sequence of SEQ ID NO:99;
g. A heavy chain variable region (VH) comprising the amino acid sequence of SEQ ID NO:103, and a light chain variable region (VL) comprising the amino acid sequence of SEQ ID NO:107;
h. A heavy chain variable region (VH) comprising the amino acid sequence of SEQ ID NO:114, and a light chain variable region (VL) comprising the amino acid sequence of SEQ ID NO:120;
i. A heavy chain variable region (VH) comprising the amino acid sequence of SEQ ID NO:55, and a light chain variable region (VL) comprising the amino acid sequence of SEQ ID NO:126;
j. A heavy chain variable region (VH) comprising the amino acid sequence of SEQ ID NO:78, and a light chain variable region (VL) comprising the amino acid sequence of SEQ ID NO:130;
k. A heavy chain variable region (VH) comprising the amino acid sequence of SEQ ID NO:90, and a light chain variable region (VL) comprising the amino acid sequence of SEQ ID NO:134;
l. A heavy chain variable region (VH) comprising the amino acid sequence of SEQ ID NO:145, and a light chain variable region (VL) comprising the amino acid sequence of SEQ ID NO:149;
m. A heavy chain variable region (VH) comprising the amino acid sequence of SEQ ID NO:162, and a light chain variable region (VL) comprising the amino acid sequence of SEQ ID NO:174;
n. A heavy chain variable region (VH) comprising the amino acid sequence of SEQ ID NO:187, and a light chain variable region (VL) comprising the amino acid sequence of SEQ ID NO:199;
o. A heavy chain variable region (VH) comprising the amino acid sequence of SEQ ID NO:212, and a light chain variable region (VL) comprising the amino acid sequence of SEQ ID NO:223;
p. A heavy chain variable region (VH) comprising the amino acid sequence of SEQ ID NO:234, and a light chain variable region (VL) comprising the amino acid sequence of SEQ ID NO:240;
q. A heavy chain variable region (VH) comprising the amino acid sequence of SEQ ID NO:249, and a light chain variable region (VL) comprising the amino acid sequence of SEQ ID NO:260;
r. A heavy chain variable region (VH) comprising the amino acid sequence of SEQ ID NO:273, and a light chain variable region (VL) comprising the amino acid sequence of SEQ ID NO:284;
s. A heavy chain variable region (VH) comprising the amino acid sequence of SEQ ID NO:288, and a light chain variable region (VL) comprising the amino acid sequence of SEQ ID NO:292; or
t. A heavy chain variable region (VH) comprising the amino acid sequence of SEQ ID NO:298, and a light chain variable region (VL) comprising the amino acid sequence of SEQ ID NO:284.

28. The immunoconjugate of claim 1, wherein the anti-DC-SIGN antibody comprises:
a. A heavy chain comprising the amino acid sequence of SEQ ID NO:12, and a light chain comprising the amino acid sequence of SEQ ID NO:23;
b. A heavy chain comprising the amino acid sequence of SEQ ID NO:36, and a light chain comprising the amino acid sequence of SEQ ID NO:47;
c. A heavy chain comprising the amino acid sequence of SEQ ID NO:57, and a light chain comprising the amino acid sequence of SEQ ID NO:66;
d. A heavy chain comprising the amino acid sequence of SEQ ID NO:36, and a light chain comprising the amino acid sequence of SEQ ID NO:72;
e. A heavy chain comprising the amino acid sequence of SEQ ID NO:80, and a light chain comprising the amino acid sequence of SEQ ID NO:86;
f. A heavy chain comprising the amino acid sequence of SEQ ID NO:92, and a light chain comprising the amino acid sequence of SEQ ID NO:101;
g. A heavy chain comprising the amino acid sequence of SEQ ID NO:105, and a light chain comprising the amino acid sequence of SEQ ID NO:109;
h. A heavy chain comprising the amino acid sequence of SEQ ID NO:116, and a light chain comprising the amino acid sequence of SEQ ID NO:122;
i. A heavy chain comprising the amino acid sequence of SEQ ID NO:57, and a light chain comprising the amino acid sequence of SEQ ID NO:128;
j. A heavy chain comprising the amino acid sequence of SEQ ID NO:80, and a light chain comprising the amino acid sequence of SEQ ID NO:132;
k. A heavy chain comprising the amino acid sequence of SEQ ID NO:92, and a light chain comprising the amino acid sequence of SEQ ID NO:136;
l. A heavy chain comprising the amino acid sequence of SEQ ID NO:147, and a light chain comprising the amino acid sequence of SEQ ID NO:151;
m. A heavy chain comprising the amino acid sequence of SEQ ID NO:164, and a light chain comprising the amino acid sequence of SEQ ID NO:176;
n. A heavy chain comprising the amino acid sequence of SEQ ID NO:189, and a light chain comprising the amino acid sequence of SEQ ID NO:201;
o. A heavy chain comprising the amino acid sequence of SEQ ID NO:214, and a light chain comprising the amino acid sequence of SEQ ID NO:225;
p. A heavy chain comprising the amino acid sequence of SEQ ID NO:236, and a light chain comprising the amino acid sequence of SEQ ID NO:242;
q. A heavy chain comprising the amino acid sequence of SEQ ID NO:251, and a light chain comprising the amino acid sequence of SEQ ID NO:262;
r. A heavy chain) comprising the amino acid sequence of SEQ ID NO:275, and a light chain comprising the amino acid sequence of SEQ ID NO:286;
s. A heavy chain comprising the amino acid sequence of SEQ ID NO:290, and a light chain comprising the amino acid sequence of SEQ ID NO:294; or
t. A heavy chain comprising the amino acid sequence of SEQ ID NO:300, and a light chain comprising the amino acid sequence of SEQ ID NO:286.

29. The immunconjugate of claim 1, wherein L is attached to the Ab via conjugation to one or more modified cysteine residues in the Ab.

30. The immunoconjugate of claim 29, wherein L is conjugated to the Ab via modified cysteine residues at positions 152 and 375 of the heavy chain of the Ab, wherein the positions are determined according to EU numbering.

31-35. (canceled)

36. The immunoconjugate of claim 1, wherein D is a compound selected from

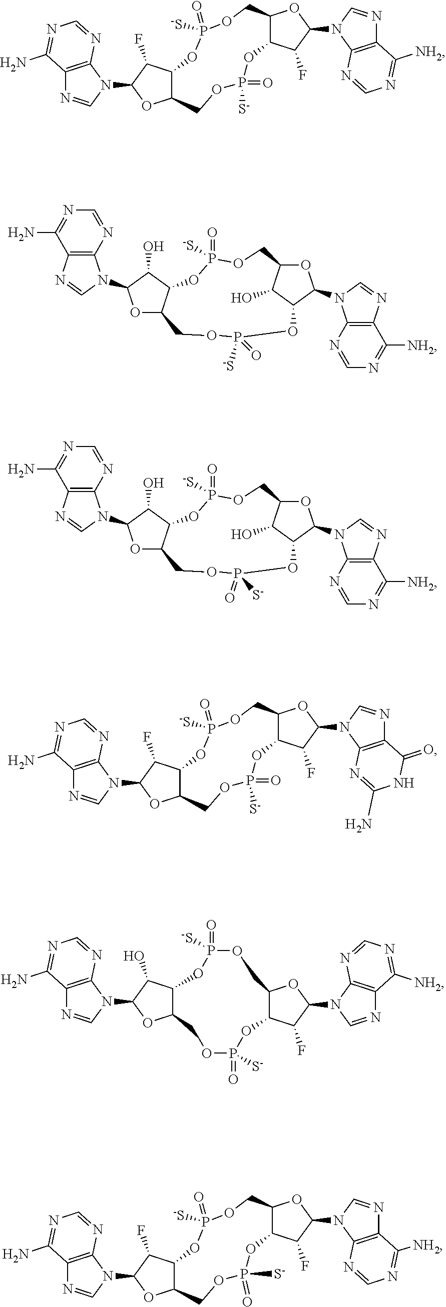

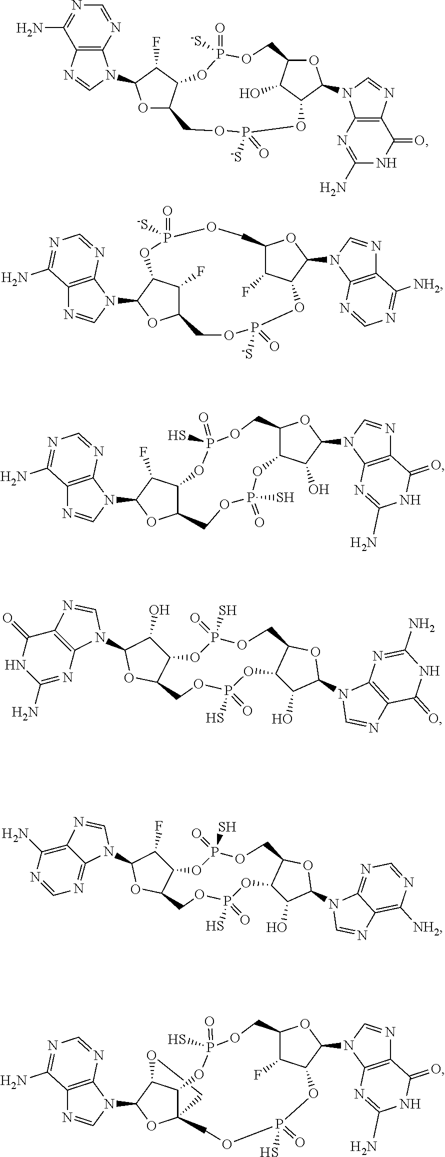

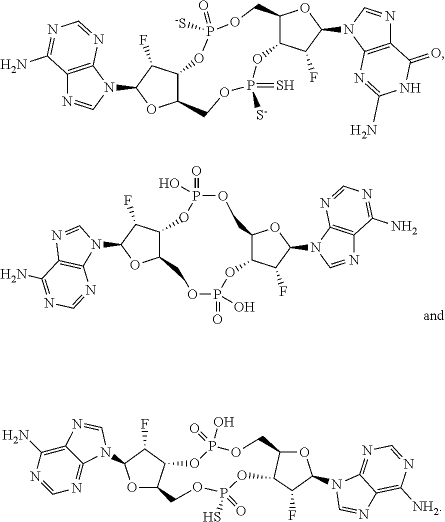

37. The immunoconjugate of claim 1,
wherein L is a cleavable linker comprising one or more cleavage elements.

38-39. (canceled)

40. The immunoconjugate of claim 37, where L has a structure selected from: 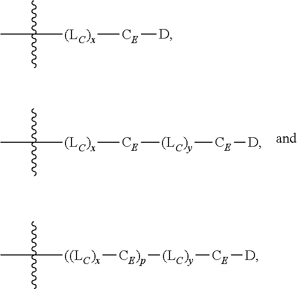
wherein:
Lc is a linker component and each Lc is independently selected from a linker component as shown in Embodiments 70 to 75;
x is an integer selected from 1, 2, 3, 4, 5, 6, 7, 8, 9, 10, 11, 12, 13, 14, 15, 16, 17, 18, 19 and 20;
y is an integer selected from 1, 2, 3, 4, 5, 6, 7, 8, 9, 10, 11, 12, 13, 14, 15, 16, 17, 18, 19 and 20;
p is an integer selected from 1, 2, 3, 4, 5, 6, 7, 8, 9 and 10;
and each cleavage element (C_{E}) is independently selected from a self-immolative spacer and a group that is susceptible to cleavage selected from acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

41. The immunoconjugate of claim 1, wherein L comprises a structure selected from:

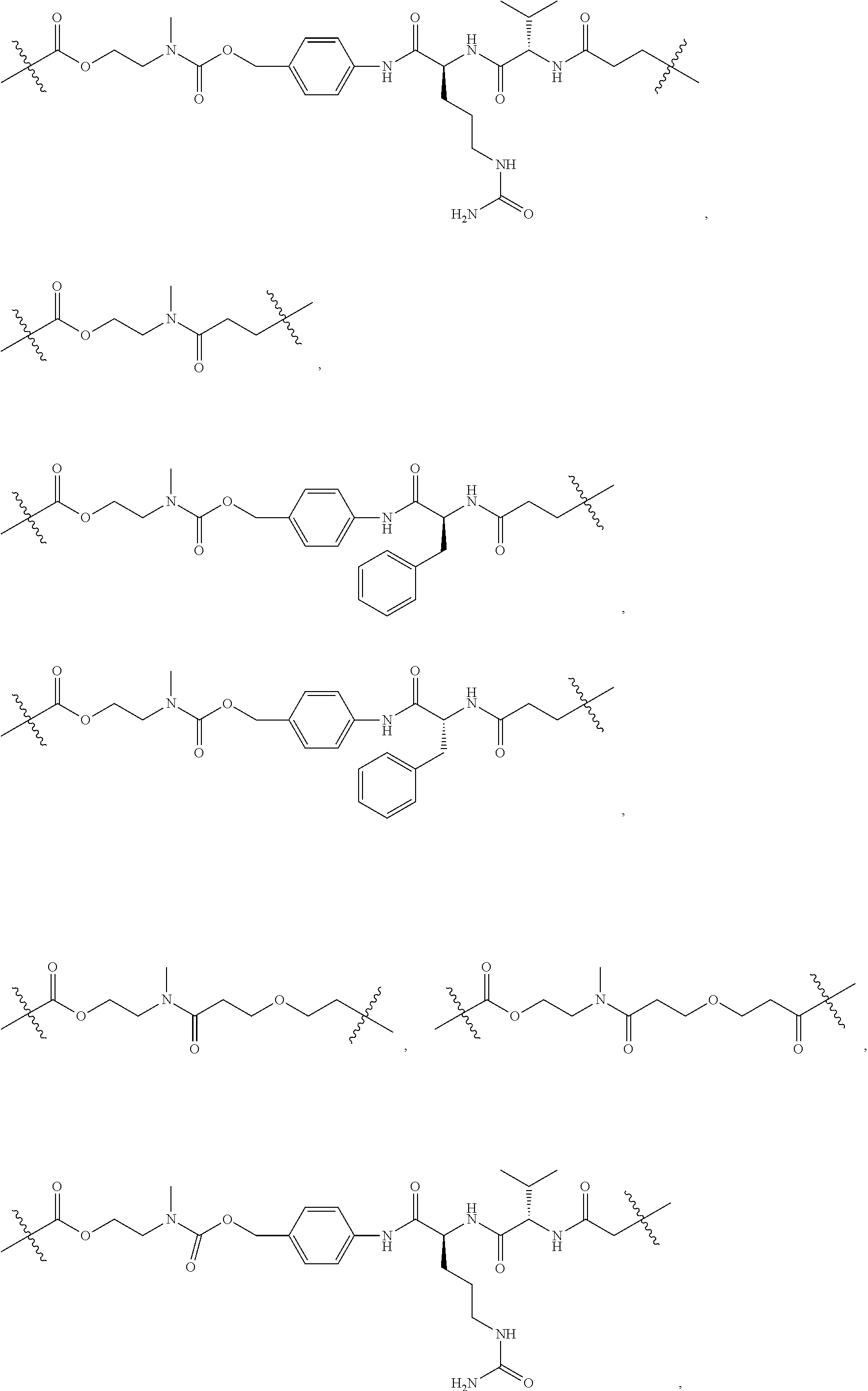

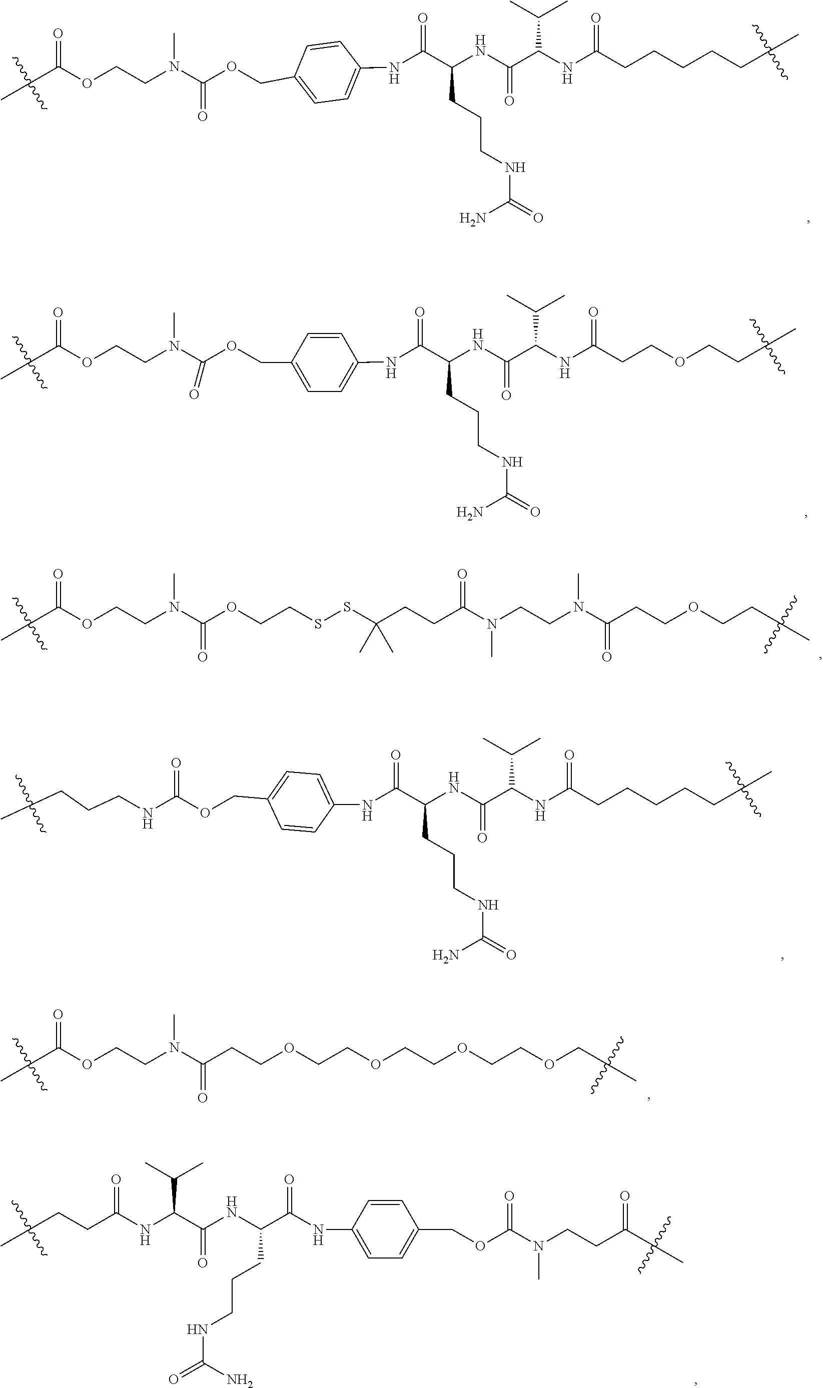

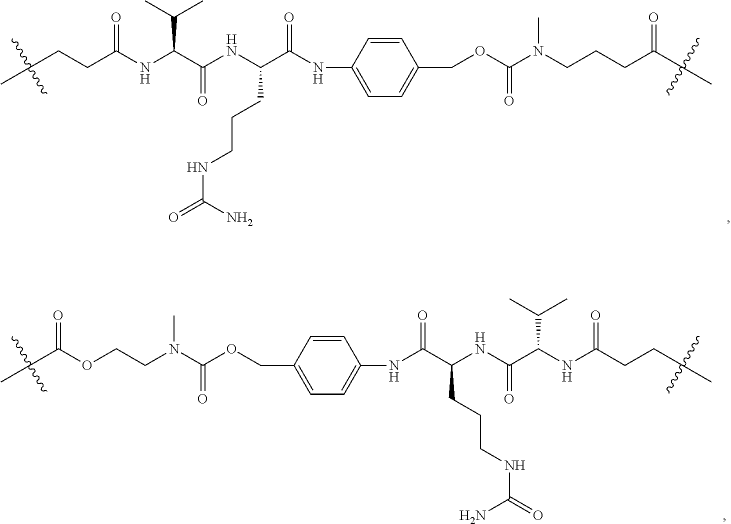 [SMILES: *CCC(=O)N[C@H](C(=O)N[C@@H](CCCNC(N)=O)C(=O)Nc1ccc(COC(=O)N(C)CCCC(*)=O)cc1)C(C)C ; *CCC(=O)N[C@H](C(=O)N[C@@H](CCCNC(N)=O)C(=O)Nc1ccc(COC(=O)N(C)CCOC(*)=O)cc1)C(C)C]

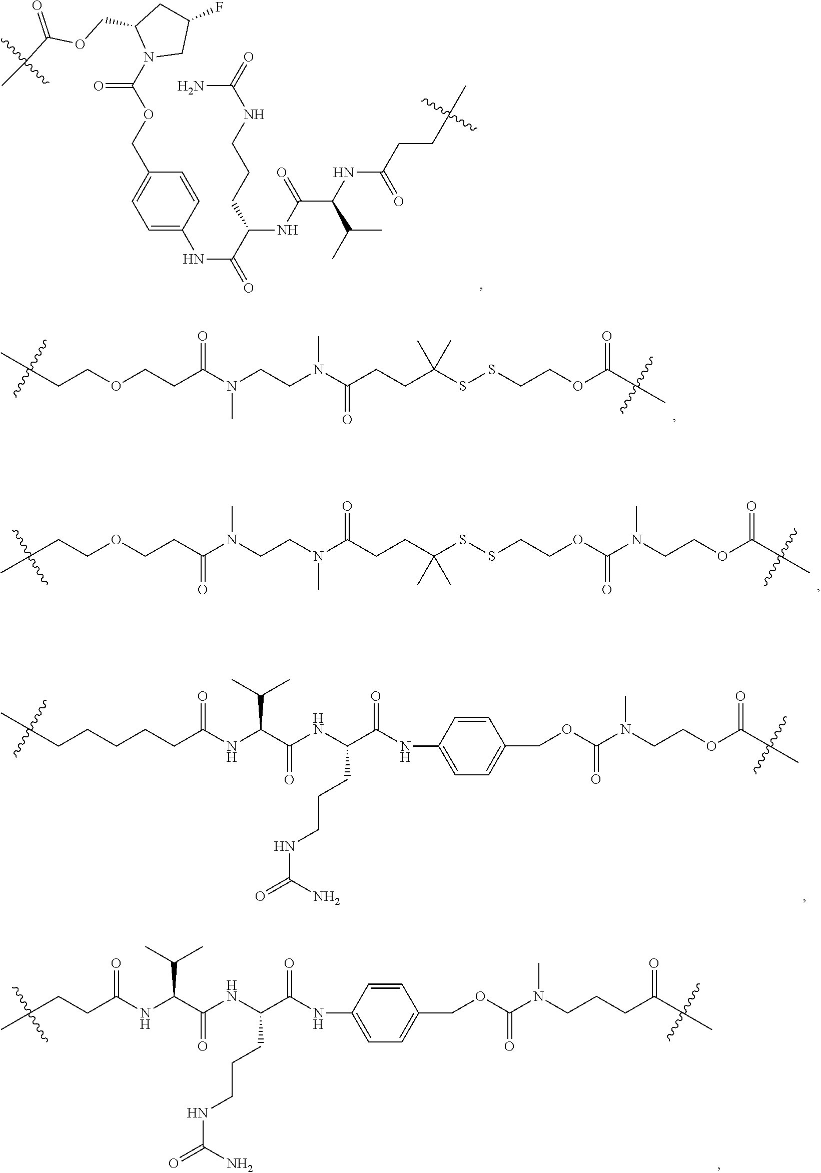 [SMILES: *CCC(=O)N[C@H](C(=O)N[C@@H](CCCNC(N)=O)C(=O)Nc1ccc(COC(=O)N2C[C@@H](F)C[C@H]2COC(*)=O)cc1)C(C)C ; *CCOCCC(=O)N(C)CCN(C)C(=O)CCC(C)(C)SSCCOC(*)=O ; *CCOCCC(=O)N(C)CCN(C)C(=O)CCC(C)(C)SSCCOC(=O)N(C)CCOC(*)=O ; *CCCCCC(=O)N[C@H](C(=O)N[C@@H](CCCNC(N)=O)C(=O)Nc1ccc(COC(=O)N(C)CCOC(*)=O)cc1)C(C)C ; *CCC(=O)N[C@H](C(=O)N[C@@H](CCCNC(N)=O)C(=O)Nc1ccc(COC(=O)N(C)CCCC(*)=O)cc1)C(C)C]

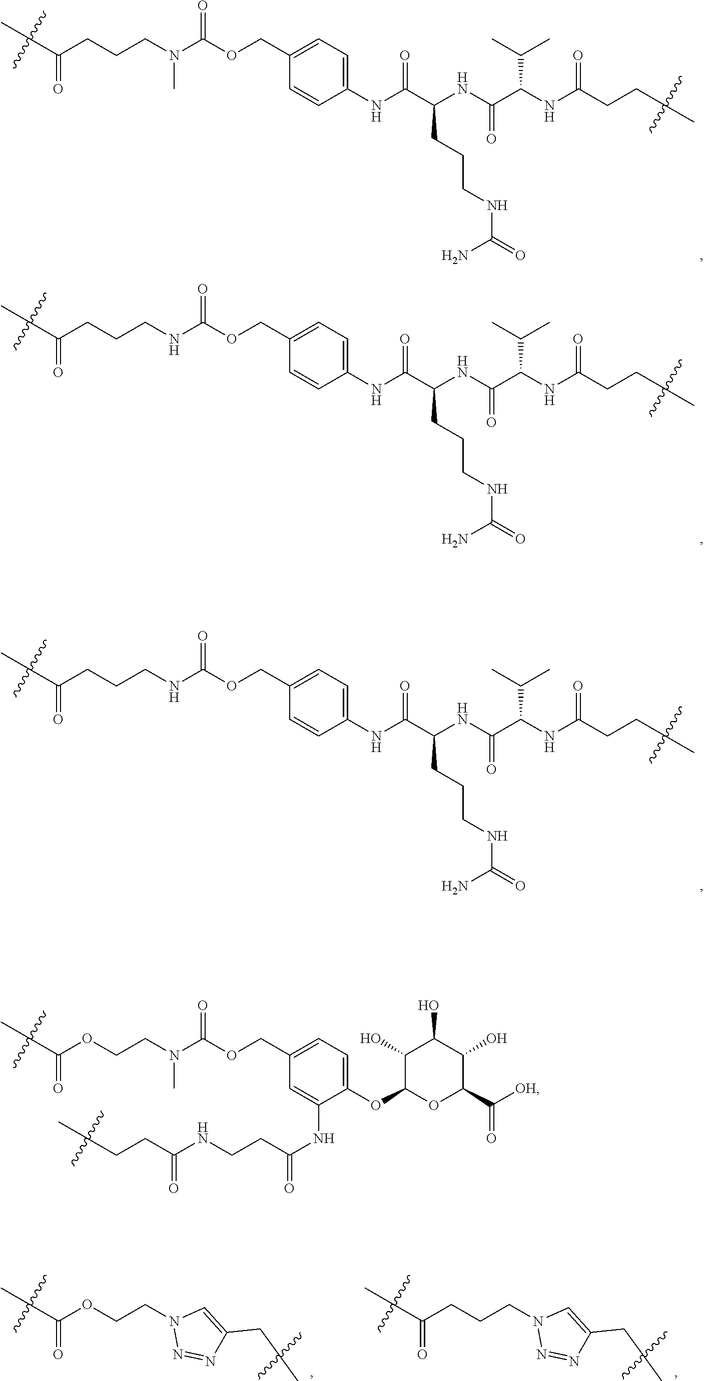 [SMILES: *CCC(=O)N[C@H](C(=O)N[C@@H](CCCNC(N)=O)C(=O)Nc1ccc(COC(=O)N(C)CCCC(*)=O)cc1)C(C)C ; *CCC(=O)N[C@H](C(=O)N[C@@H](CCCNC(N)=O)C(=O)Nc1ccc(COC(=O)NCCCC(*)=O)cc1)C(C)C ; *CCC(=O)N[C@H](C(=O)N[C@@H](CCCNC(N)=O)C(=O)Nc1ccc(COC(=O)NCCCC(*)=O)cc1)C(C)C ; *CCC(=O)NCCC(=O)Nc1cc(COC(=O)N(C)CCOC(*)=O)ccc1O[C@@H]1O[C@H](C(=O)O)[C@@H](O)[C@H](O)[C@H]1O ; *Cc1cn(CCOC(*)=O)nn1 ; *Cc1cn(CCCC(*)=O)nn1]

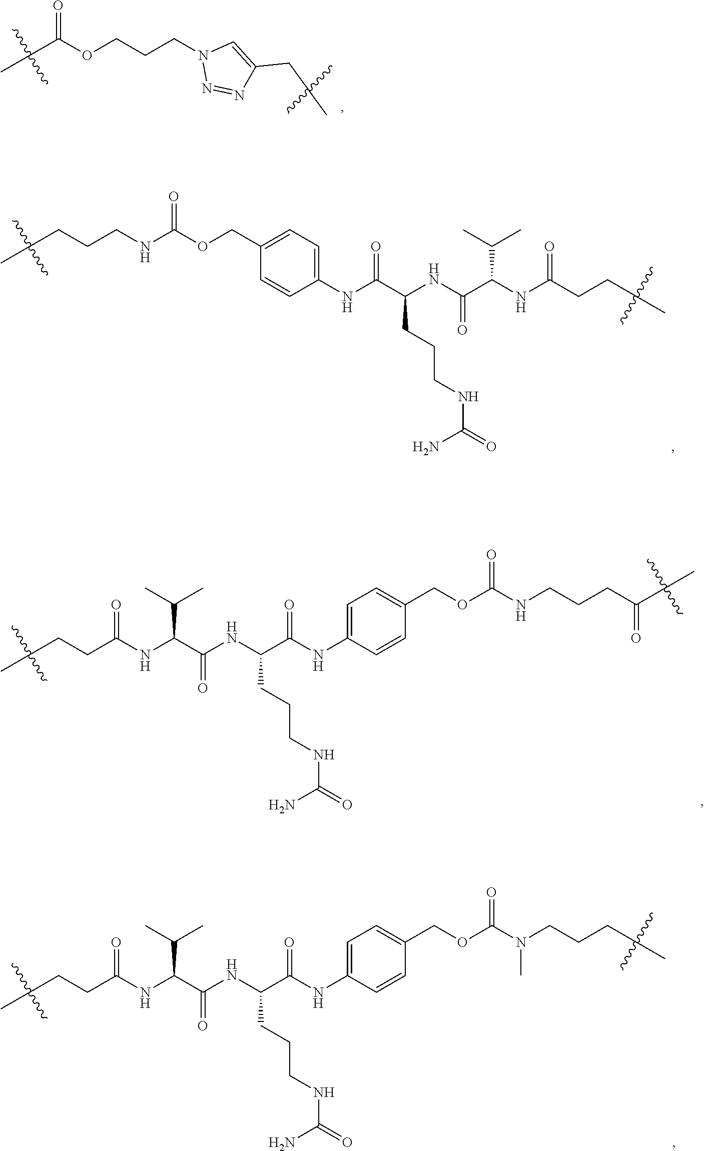 [SMILES: *Cc1cn(CCCOC(*)=O)nn1 ; *CCCNC(=O)OCc1ccc(NC(=O)[C@H](CCCNC(N)=O)NC(=O)[C@@H](NC(=O)CC*)C(C)C)cc1 ; *CCC(=O)N[C@H](C(=O)N[C@@H](CCCNC(N)=O)C(=O)Nc1ccc(COC(=O)NCCCC(*)=O)cc1)C(C)C ; *CCCN(C)C(=O)OCc1ccc(NC(=O)[C@H](CCCNC(N)=O)NC(=O)[C@@H](NC(=O)CC*)C(C)C)cc1]

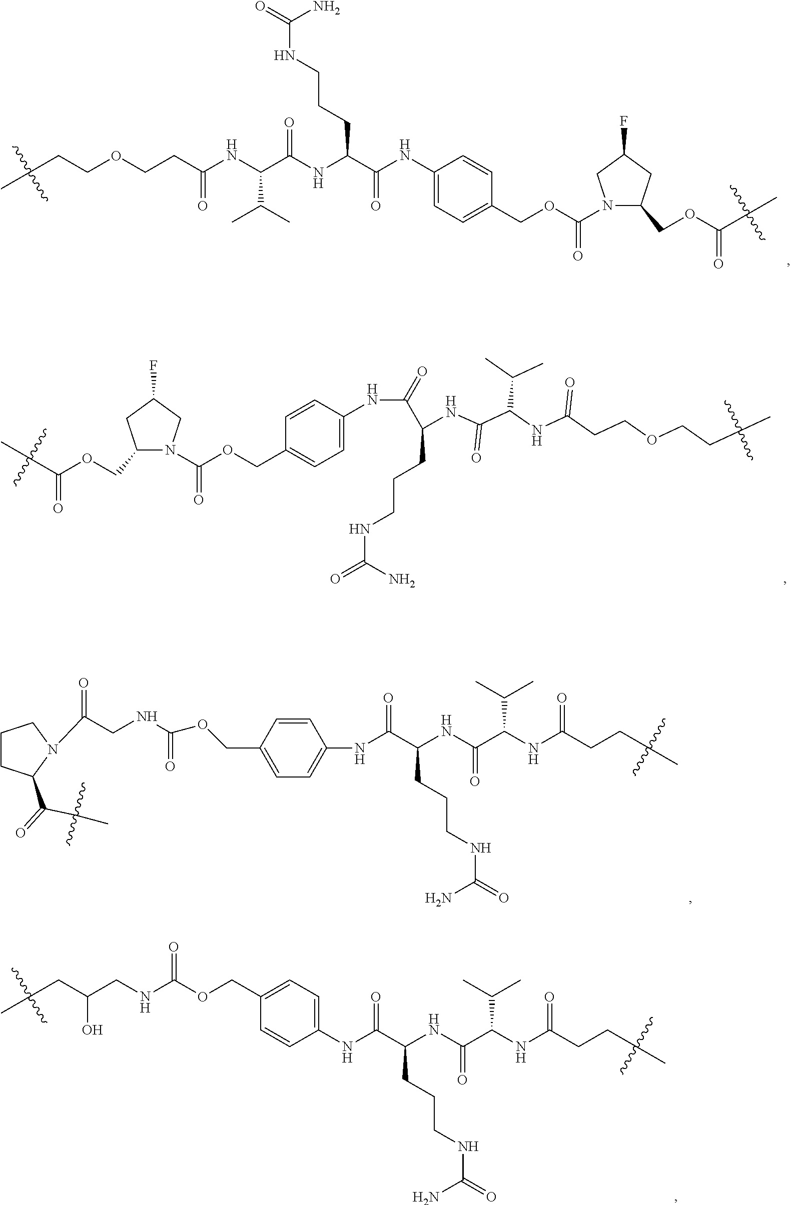

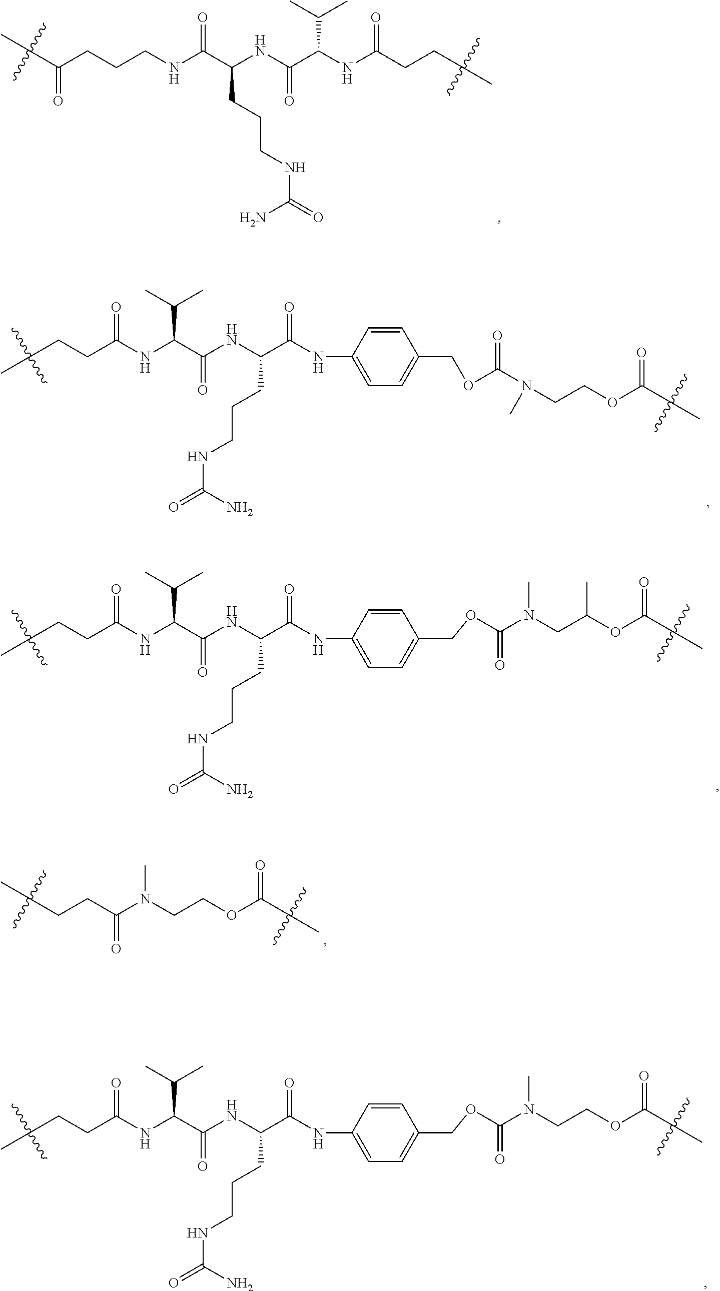 [SMILES: *CCC(=O)N[C@H](C(=O)N[C@@H](CCCNC(N)=O)C(=O)NCCCC(*)=O)C(C)C ; *CCC(=O)N[C@H](C(=O)N[C@@H](CCCNC(N)=O)C(=O)Nc1ccc(COC(=O)N(C)CCOC(*)=O)cc1)C(C)C ; *CCC(=O)N[C@H](C(=O)N[C@@H](CCCNC(N)=O)C(=O)Nc1ccc(COC(=O)N(C)CC(C)OC(*)=O)cc1)C(C)C ; *CCC(=O)N(C)CCOC(*)=O ; *CCC(=O)N[C@H](C(=O)N[C@@H](CCCNC(N)=O)C(=O)Nc1ccc(COC(=O)N(C)CCOC(*)=O)cc1)C(C)C]

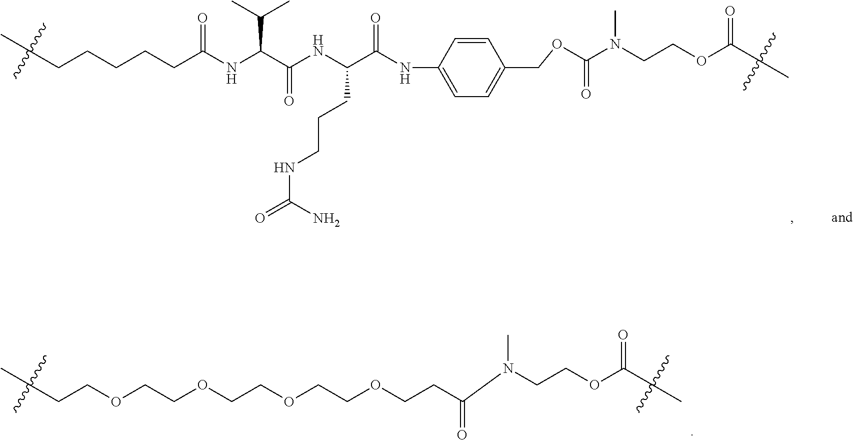 [SMILES: *CCCCCC(=O)N[C@H](C(=O)N[C@@H](CCCNC(N)=O)C(=O)Nc1ccc(COC(=O)N(C)CCOC(*)=O)cc1)C(C)C ; *CCOCCOCCOCCOCCC(=O)N(C)CCOC(*)=O]

42. The immunoconjugate of claim 1, wherein the immunoconjugate is selected from the following: 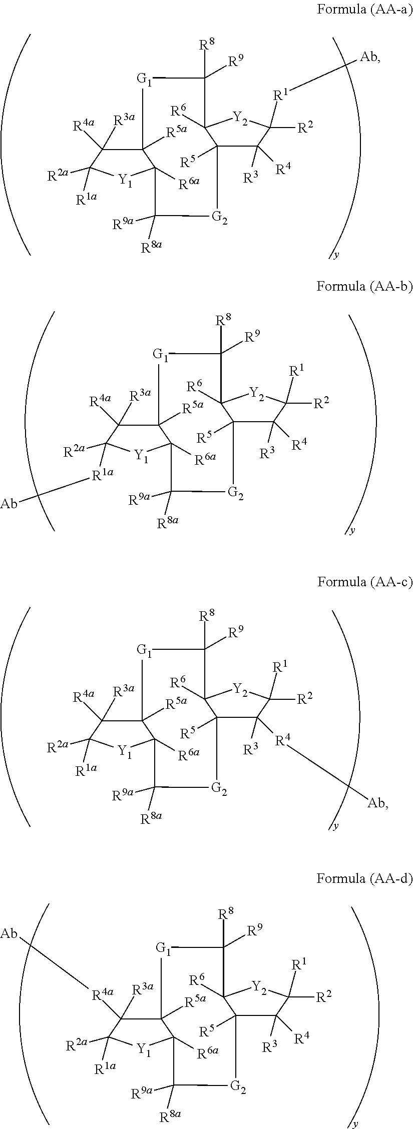 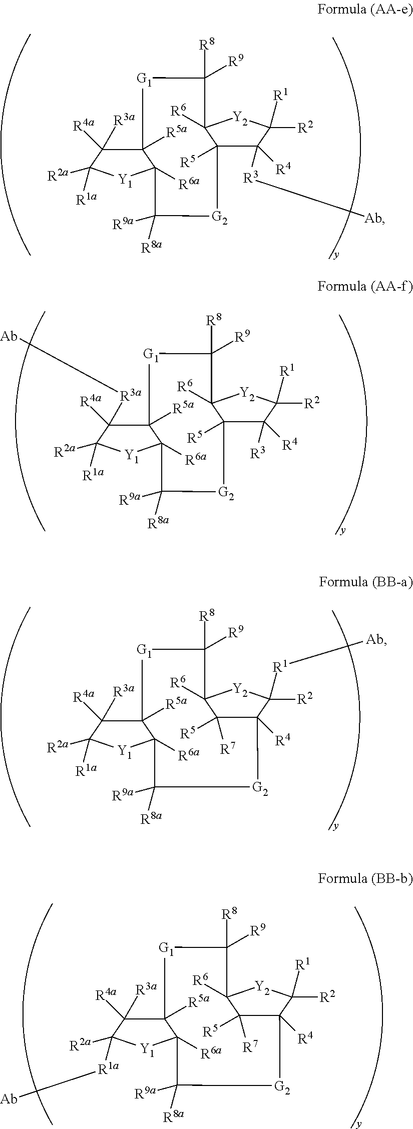 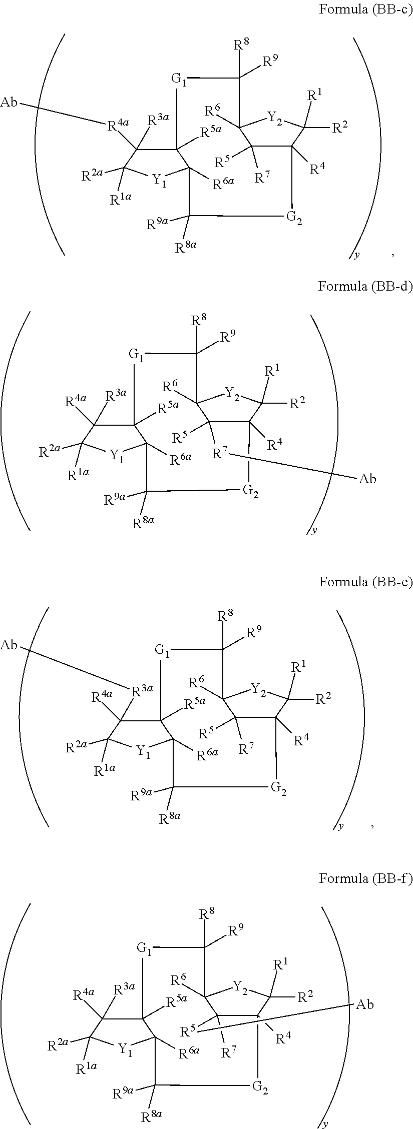 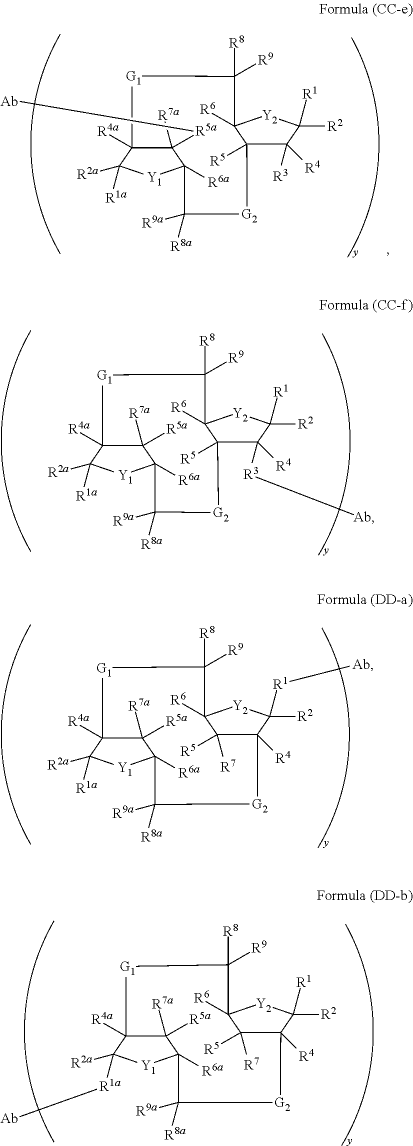 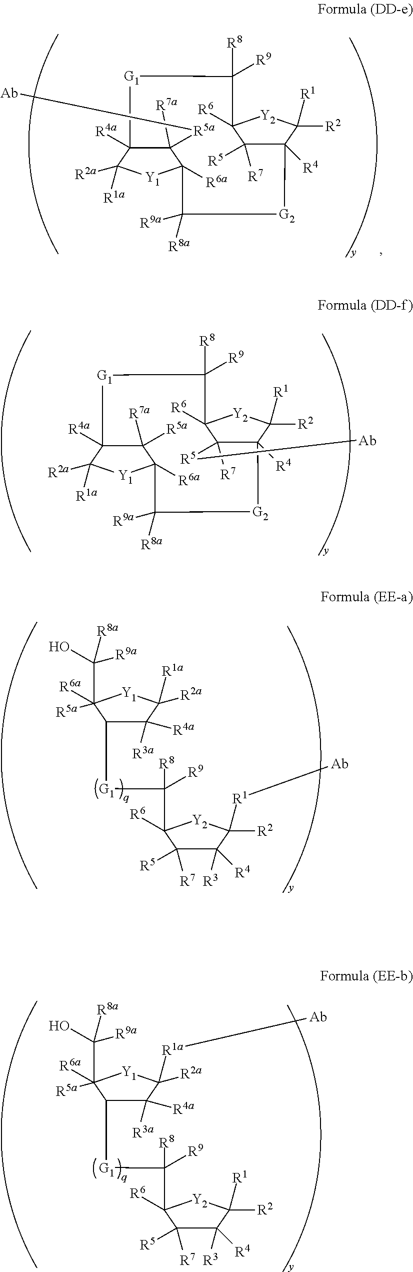 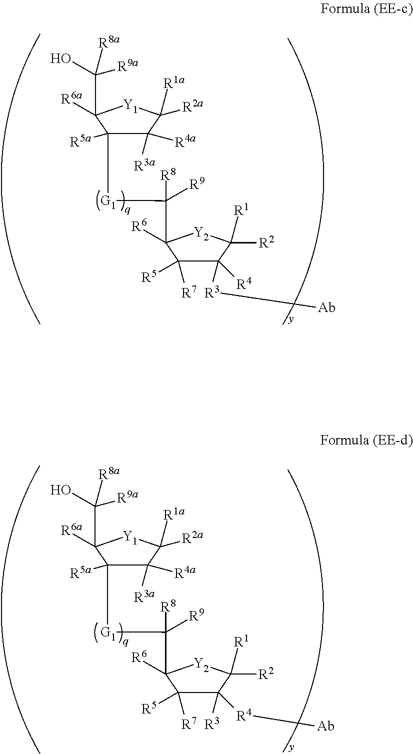 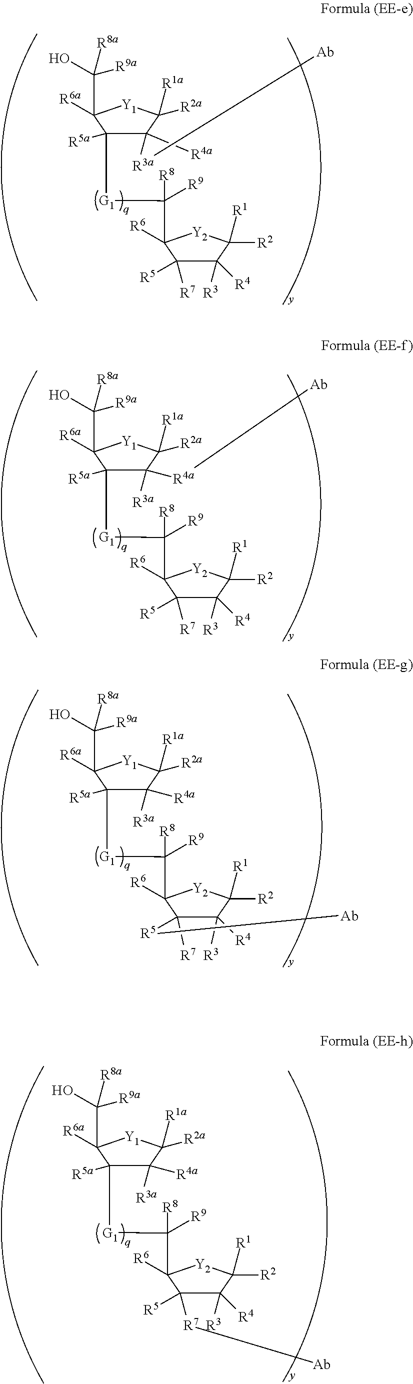 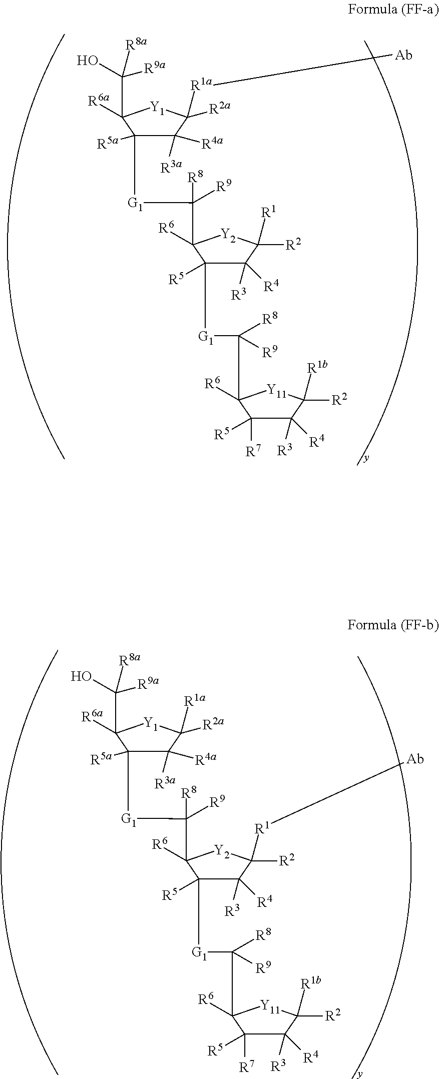 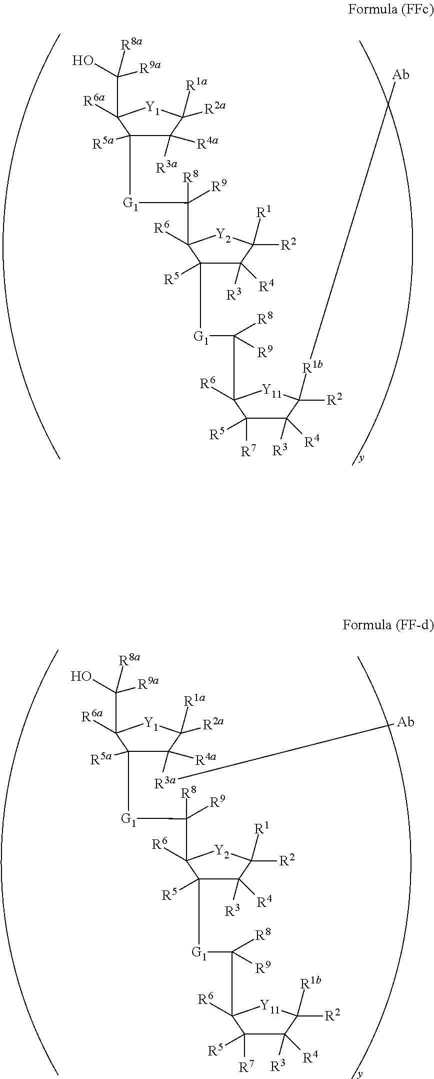 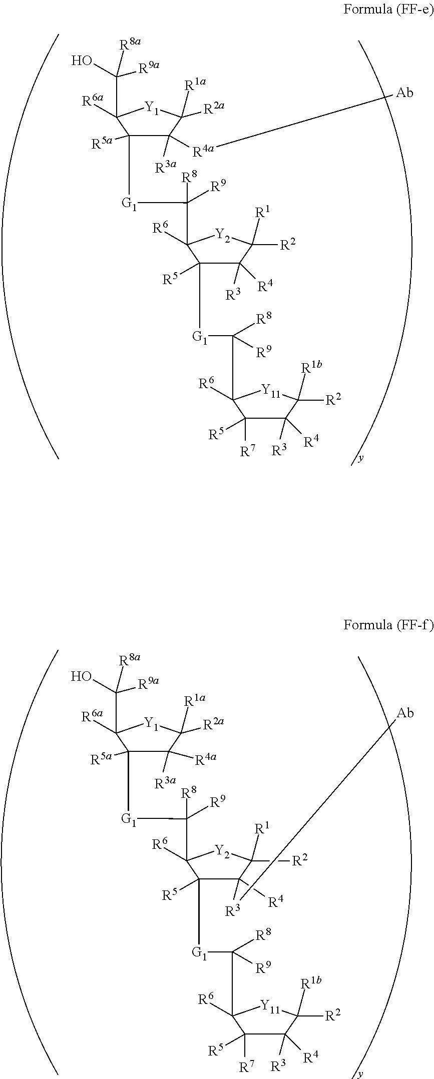 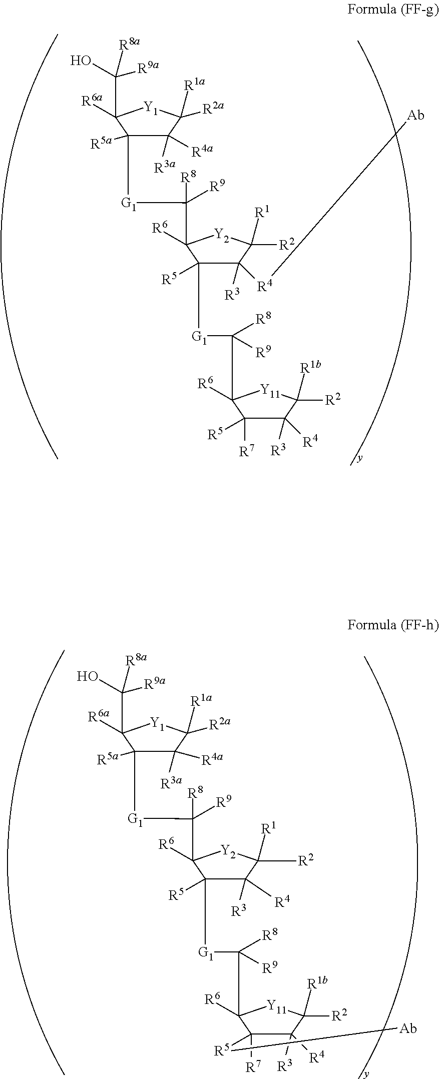 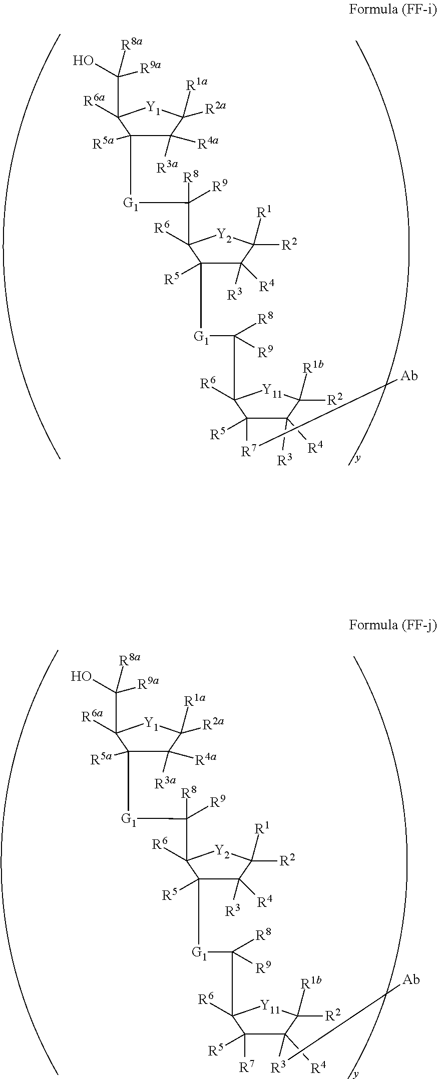 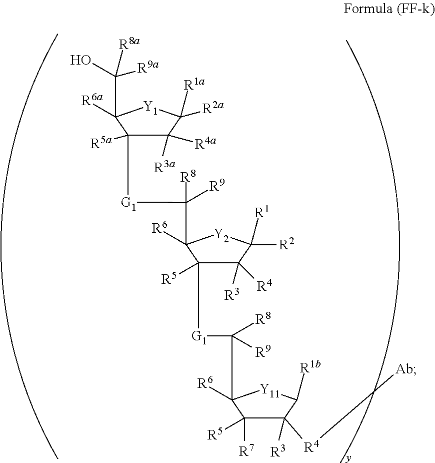
wherein:
each G₁ is independently selected from 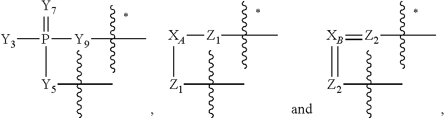
where the * of G₁ indicates the point of attachment to —CR⁸R⁹—;
X_{A} is C(═O)—, —C(═S)— or —C(═NR¹¹)— and each Z₁ is NR¹²;
X_{B} is C, and each Z₂ is N;
G₂ is 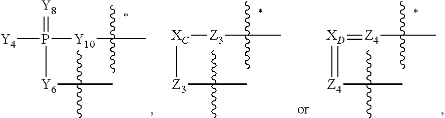
where the * of G₂ indicates the point of attachment to —CR^{8a}R^{9a}—;
X_{C} is C(═O)—, —C(═S)— or —C(═NR¹¹)— and each Z₃ is NR¹²;
X_{D} is C, and each Z₄ is N;
Y₁ is —O—, —S—, —S(═O)—, —SO₂—, —CH₂—, or —CF₂—;
Y₂ is —O—, —S—, —S(═O)—, —SO₂—, —CH₂—, or —CF₂—;
Y₃ is OH, O⁻, OR¹⁰, N(R¹⁰)₂, SR¹, SeH, Se⁻, BH₃, SH or S⁻;
Y₄ is OH, O⁻, OR¹⁰, N(R¹⁰)₂, SR¹⁰, SeH, Se⁻, BH₃, SH or S⁻;
Y₅ is —CH₂—, —NH—, —O— or —S;
Y₆ is —CH₂—, —NH—, —O— or —S;
Y₇ is O or S;
Y₈ is O or S;
Y₉ is —CH₂—, —NH—, —O— or —S;
Y₁₀ is —CH₂—, —NH—, —O— or —S;
Y₁₁ is —O—, —S—, —S(═O)—, —SO₂—, —CH₂—, or —CF₂—;
q is 1,2 or 3;
each R¹ is independently a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R¹ is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R^{1a} is independently a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1a} is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R^{1b} is independently a partially saturated or aromatic monocyclic heterocyclyl or partially saturated or aromatic fused bicyclic heterocyclyl containing from 5-10 ring members selected from carbon atoms and 1 to 5 heteroatoms, and each heteroatoms is independently selected from O, N or S, or a tautomer thereof, wherein R^{1b} is substituted with 0, 1, 2, 3 or 4 substituents independently selected from —NHL₁R¹¹⁵, F, Cl, Br, OH, SH, NH₂, D, CD₃, C₁-C₆alkyl, C₁-C₆alkoxyalkyl, C₁-C₆hydroxyalkyl, C₃-C₈cycloalkyl, a 3 to 6 membered heterocyclyl having 1 to 2 heteroatoms independently selected from O, N and S, —O(C₁-C₆alkyl), —O(C₃-C₈cycloalkyl), —S(C₁-C₆alkyl), —S(C₁-C₆aminoalkyl), —S(C₁-C₆hydroxyalkyl), —S(C₃-C₈cycloalkyl), —NH(C₁-C₆alkyl), —NH(C₃-C₈cycloalkyl), —N(C₁-C₆alkyl)₂, —N(C₁-C₆alkyl) (C₃-C₈cycloalkyl), —CN, —P(═O)(OH)₂, —O(CH₂)₁₋₁₀C(═O)OH, —(CH₂)₁₋₁₀C(═O)OH, —CH═CH(CH₂)₁₋₁₀C(═O)OH, —NHC(O)(C₁-C₆alkyl), —NHC(O)(C₃-C₈cycloalkyl), —NHC(O)(phenyl), and —N(C₃-C₈cycloalkyl)₂;
each R² is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R² and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R² are substituted by 0, 1, 2 or 3 substituents independently selected from F, Cl, Br, I, OH, CN, and N₃;
each R³ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₅alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R³ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C6alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R³ are substituted by 0, 1, 2 or 3 substituents independently selected from F, Cl, Br, I, OH, CN, and N₃;
each R⁴ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁴ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁴ are substituted by 0, 1, 2 or 3 substituents independently selected from F, Cl, Br, I, OH, CN, and N₃;
each R⁵ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁵ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁵ are substituted by 0, 1, 2 or 3 substituents independently selected from F, Cl, Br, I, OH, CN, and N₃;
each R⁶ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁶ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁶ are substituted by 0, 1, 2 or 3 substituents independently selected from F, Cl, Br, I, OH, CN, and N₃;
each R⁷ is independently selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁷ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁷ are substituted by 0, 1, 2 or 3 substituents independently selected from F, Cl, Br, I, OH, CN, and N₃;
each R⁸ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁸ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R⁹ is independently selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R⁹ and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁹ are substituted by 0, 1, 2 or 3 substituents independently selected from F, Cl, Br, I, OH, CN, and N₃;
each R^{2a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{2a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{2a} are substituted by 0, 1, 2 or 3 substituents independently selected from F, Cl, Br, I, OH, CN, and N₃
each R^{3a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{3a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{3a} are substituted by 0, 1, 2 or 3 substituents independently selected from F, Cl, Br, I, OH, CN, and N₃;
each R^{4a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{4a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{4a} are substituted by 0, 1, 2 or 3 substituents independently selected from F, Cl, Br, I, OH, CN, and N₃;
each R^{5a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{5a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{5a} are substituted by 0, 1, 2 or 3 substituents independently selected from F, Cl, Br, I, OH, CN, and N₃;
each R^{6a} is selected from the group consisting of H, —OH, F, Cl, Br, I, D, CD₃, CN, N₃, C₁-C₆alkyl, C₂-C₆alkenyl, C₂-C₆alkynyl, C₁-C₅haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₅alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{6a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{6a} are substituted by 0, 1, 2 or 3 substituents independently selected from F, Cl, Br, I, OH, CN, and N₃;
each R^{7a} is selected from the group consisting of —OL₁R¹¹⁵, H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₅alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{7a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{7a} are substituted by 0, 1, 2 or 3 substituents independently selected from F, Cl, Br, I, OH, CN, and N₃;
each R^{8a} is selected from the group consisting of H, —OH, F, Cl, Br, I, D, CD₃, CN, N₃, C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{8a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₆alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R⁸ are substituted by 0, 1, 2 or 3 substituents independently selected from F, Cl, Br, I, OH, CN, and N₃;
each R^{9a} is selected from the group consisting of H, —OH, F, Cl, Br, I, D, CD₃, CN, N₃, C₁-C₆alkyl, C₂-C₆alkenyl, C₂-C₆alkynyl, C₁-C₅haloalkyl, C₂-C₆haloalkenyl, C₂-C₆haloalkynyl, —O(C₁-C₆alkyl), —O(C₂-C₆alkenyl), —O(C₂-C₆alkynyl), —OP(═O)(OH)₂, —O(CH₂)₁₋₁₀C(═O)OH, —O(CH₂)₁₋₁₀P(═O)(OH)₂, —OC(O)Ophenyl, —OC(O)OC₁-C₆alkyl, —OC(O)OC₂-C₆alkenyl, —OC(O)OC₂-C₆alkynyl, —OC(O)phenyl, —OC(O)C₁-C₆alkyl, —OC(O)C₂-C₆alkenyl and —OC(O)C₂-C₆alkynyl, wherein the —OC(O)Ophenyl of R^{9a} and the C₁-C₆alkyl, C₂-C₆alkenyl and C₂-C₆alkynyl of the C₁-C₆alkyl, C₂-C₆alkenyl, C₂-C₆alkynyl, C₁-C₆haloalkyl, C₂-C₆haloalkenyl, C₂-C₆haloalkynyl, —O(C₁-C₆alkyl), —O(C₂-C₆alkenyl), —O(C₂-C₆alkynyl), —OC(O)OC₁-C₅alkyl, —OC(O)OC₂-C₆alkenyl, —OC(O)OC₂-C₆alkynyl, —OC(O)C₁-C₆alkyl, —OC(O)C₂-C₆alkenyl and —OC(O)C₂-C₆alkynyl of R^{9a} are substituted by 0, 1, 2 or 3 substituents independently selected from F, Cl, Br, I, OH, CN, and N₃;
each R¹⁰ is independently selected from the group consisting of H, C₁-C₁₂alkyl, C₁-C₆heteroalkyl, —(CH₂CH₂O)ₙCH₂CH₂C(═O)OC₁-C₆alkyl, and 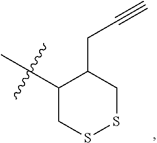 [SMILES: *C1CSSCC1CC#C]
wherein the C₁-C₁₂alkyl and C₁-C₆heteroalkyl of R¹⁰ is substituted by 0, 1, 2 or 3 substituents independently selected from —OH, C₁-C₁₂alkoxy, —S—C(═O)C₁-C₆alkyl, halo, —CN, C₁-C₁₂alkyl, —O-aryl, _O-heteroaryl, —O-cycloalkyl, oxo, cycloalkyl, heterocyclyl, aryl, or heteroaryl, —OC(O)OC₁-C₆alkyland C(O)OC₁-C₆alkyl, wherein each alkyl, cycloalkyl, heterocyclyl, aryl, and heteroaryl is substituted by 0, 1, 2 or 3 substituents independently selected from C₁-C₁₂ alkyl, O—C₁-C₁₂alkyl, C₁-C₁₂heteroalkyl, halo, CN, OH, oxo, aryl, heteroaryl, O-aryl, O-heteroaryl, —C(═O)C₁-C₁₂alkyl, —OC(═O)C₁-C₁₂alkyl, —C(═O)OC₁-C₁₂alkyl, —OC(═O)OC₁-C₁₂alkyl, —C(═O)N(R¹¹)—C₁-C₁₂alkyl, —N(R¹¹)C(═O)—C₁-C₁₂alkyl; —OC(═O)N(R¹¹)—C₁-C₁₂alkyl, —C(═O)-aryl, —C(═O)-heteroaryl, —OC(═O)-aryl, —C(═O)O-aryl, —OC(═O)-heteroaryl, —C(═O)O-heteroaryl, —C(═O)O-aryl, —C(═O)O-heteroaryl, —C(═O)N(R¹¹)-aryl, —C(═O)N(R¹¹)-heteroaryl, —N(R¹¹)C(O)-aryl, —N(R¹¹)₂C(O)-aryl, —N(R¹¹)C(O)-heteroaryl, and S(O)₂N(R¹¹)-aryl;
each R¹¹ is independently selected from H and C₁-C₆alkyl;
each R¹² is independently selected from H and C₁-C₆alkyl;
optionally R³ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R³ and R⁶ are connected, the O is bound at the R³ position
optionally R^{3a} and R^{6a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{3a} and R^{6a} are connected, the O is bound at the R^{3a} position;
optionally R² and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R² and R³ are connected, the O is bound at the R³ position;
optionally R^{2a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{2a} and R^{3a} are connected, the O is bound at the R^{3a} position;
optionally R⁴ and R³ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁴ and R³ are connected, the O is bound at the R³ position;
optionally R^{4a} and R^{3a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{4a} and R^{3a} are connected, the O is bound at the R^{3a} position;
optionally R⁵ and R⁶ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁶ are connected, the O is bound at the R⁵ position;
optionally R^{5a} and R^{6a}, are connected to form C₁-C₅alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{6a} are connected, the O is bound at the R^{5a} position;
optionally R⁵ and R⁷ are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R⁵ and R⁷ are connected, the O is bound at the R⁵ position;
optionally R^{5a} and R^{7a}, are connected to form C₁-C₆alkylene, C₂-C₆alkenylene, C₂-C₆alkynylene, —O—C₁-C₆alkylene, —O—C₂-C₆alkenylene, —O—C₂-C₆alkynylene, such that when R^{5a} and R^{7a} are connected, the O is bound at the R^{5a} position;
optionally R⁸ and R⁹ are connected to form a C₁-C₆alkylene, C₂-C₆alkenylene, C₂-C₆alkynylene, and
optionally R^{8a} and R^{9a} are connected to form a C₁-C₆alkylene, C₂-C₆alkenylene, C₂-C₆alkynylene,
L₁ is a linker;
each R¹¹⁵ is independently 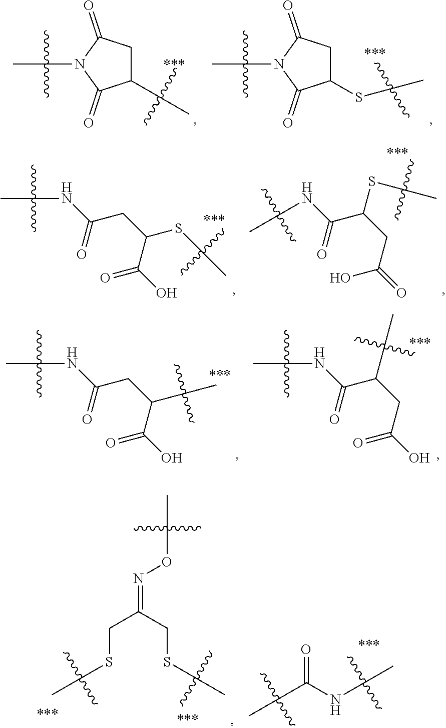
—C(═O)—, —ON═***, —S—, —NHC(═O)CH₂—***, —S(═O)₂CH₂CH₂—***, —(CH₂)₂S(═O)₂CH₂CH₂—***, —NHS(═O)₂C₂CH_{2-**}, —NHC(═O)CH₂CH₂—***, —CH₂NHCH₂CH₂—***, —NHCH₂CH₂—***, 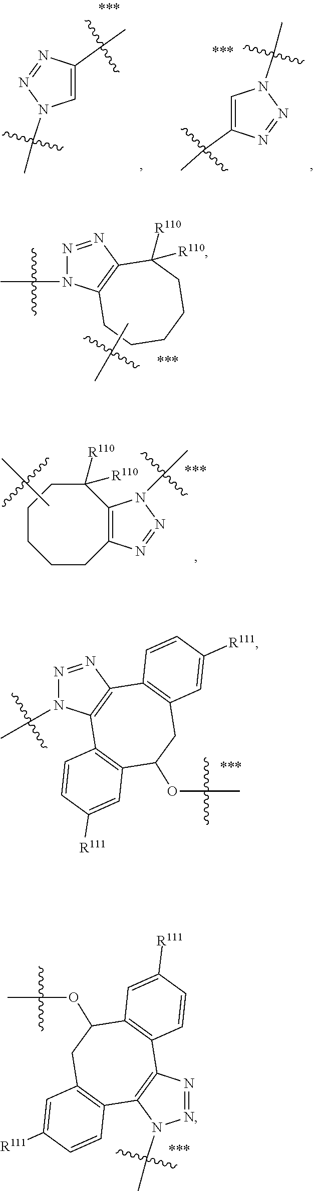 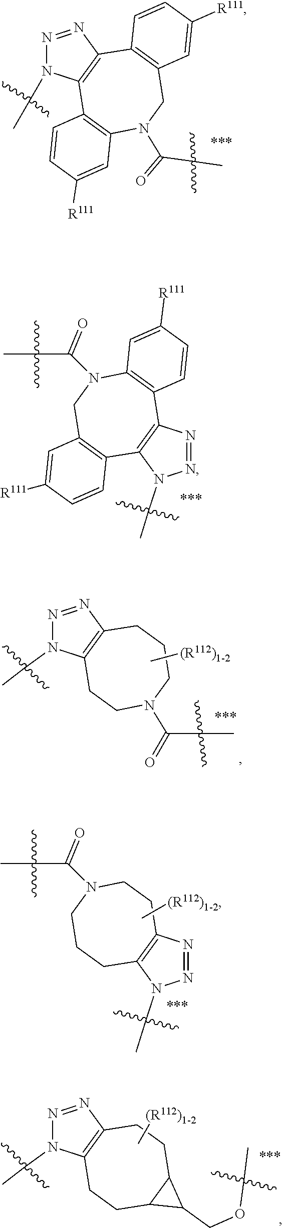 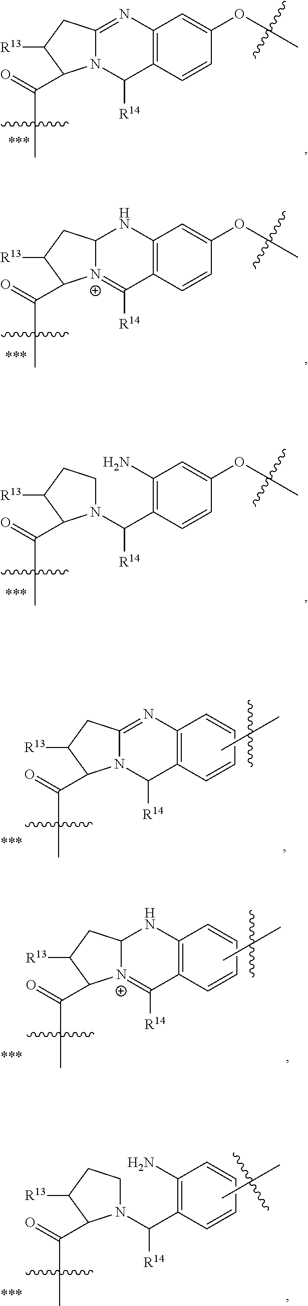 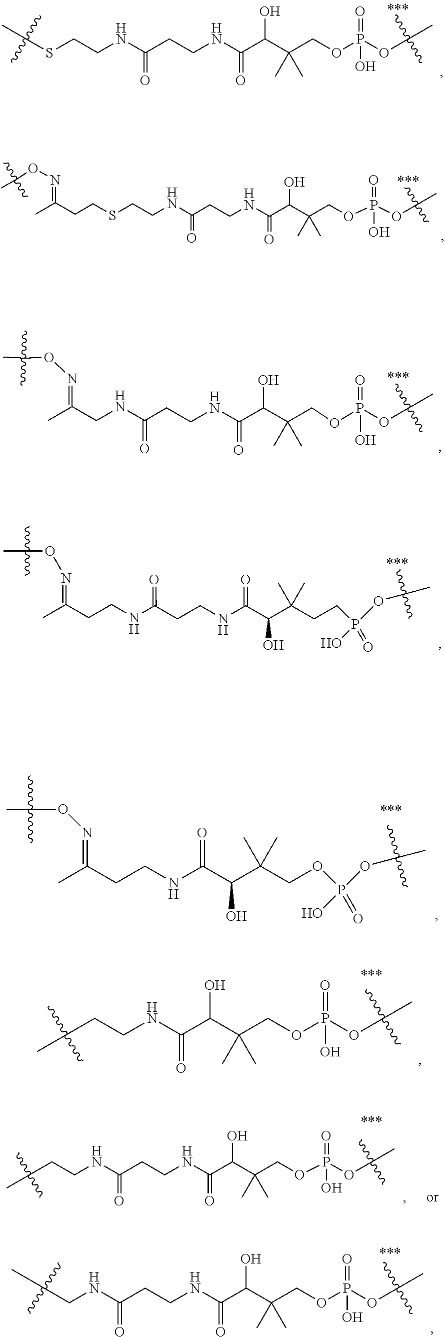
where the *** of R¹¹⁵ indicates the point of attachment to Ab;
R¹³ is H or methyl;
R¹⁴ is H, —CH₃ or phenyl;
each R¹¹⁰ is independently selected from H, C₁-C₆alkyl, F, Cl, and —OH;
each R¹¹¹ is independently selected from H, C₁-C₆alkyl, F, Cl, —NH₂, —OCH₃, —OCH₂CH₃, —N(CH₃)₂, —CN, —NO₂ and —OH;
each R¹¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
Ab is an anti-DC-SIGN antibody or a functional fragment thereof; and
y is 1, 2, 3, 4, 5, 6, 7, 8, 9 or 10.

43. The immunoconjugate of claim 1 comprising a structure selected from:

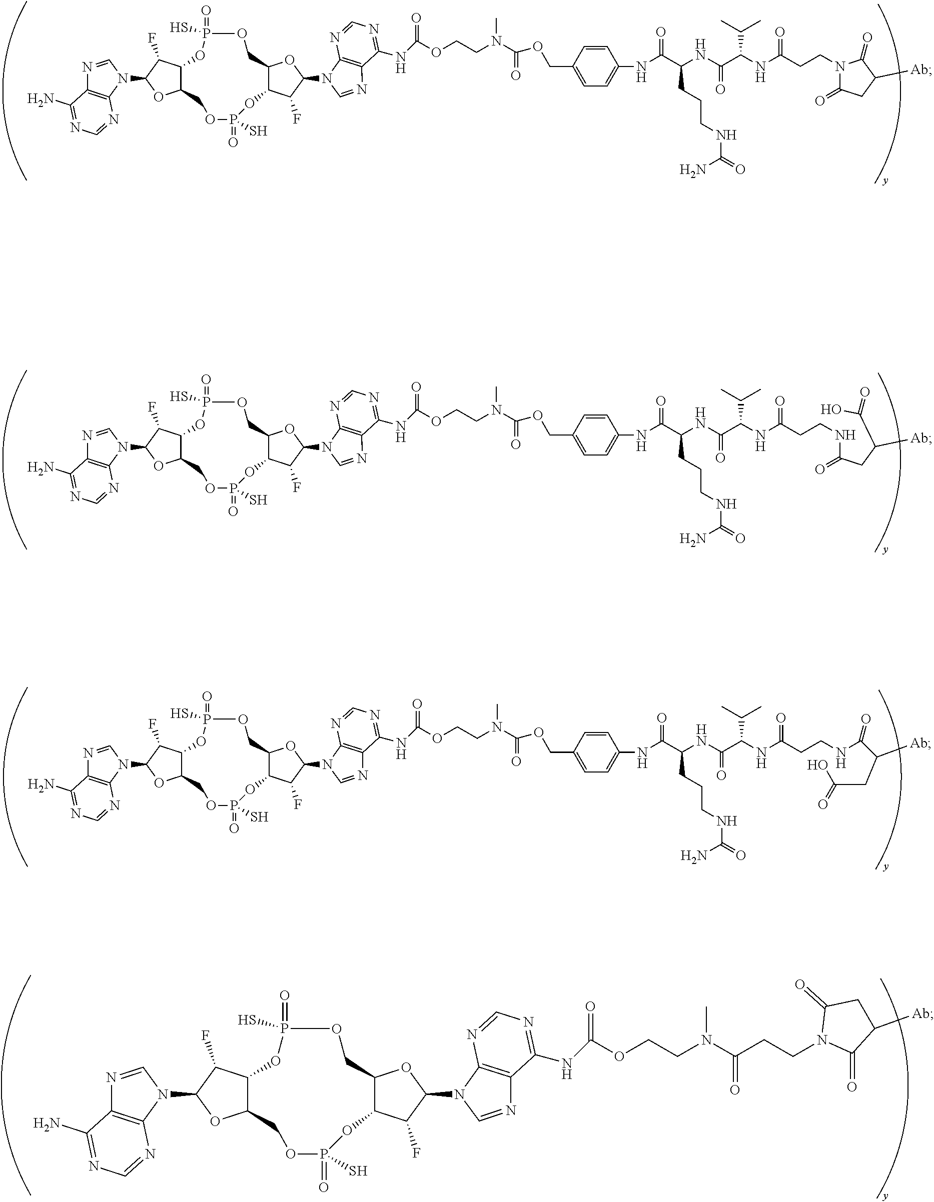

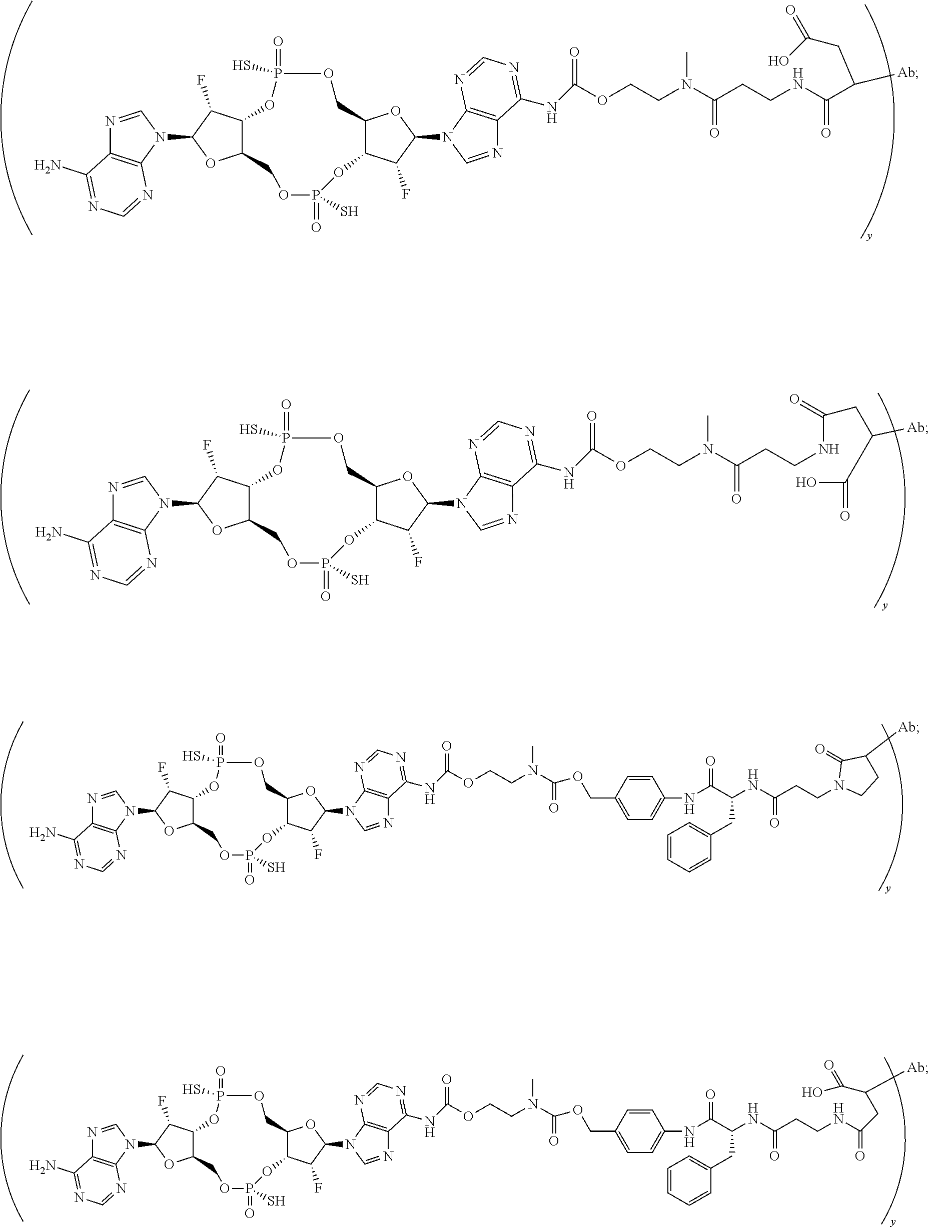

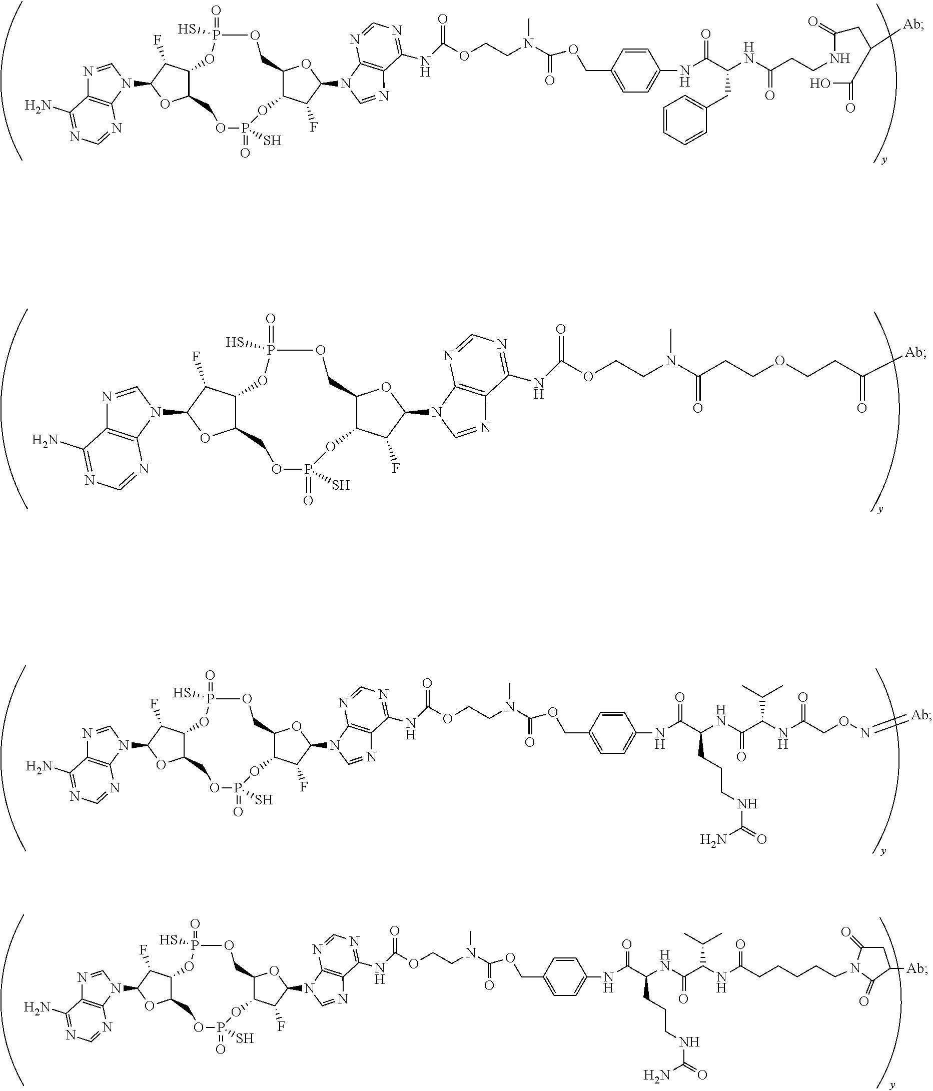

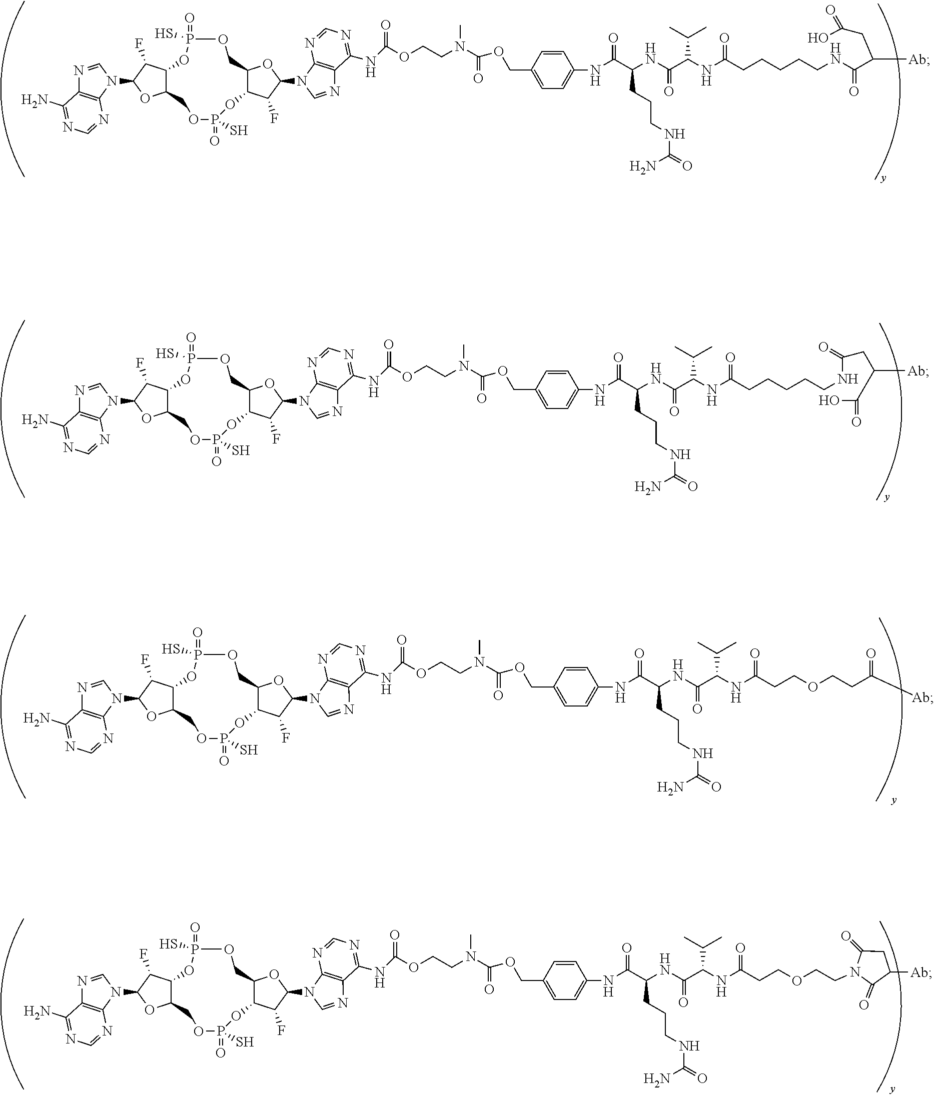

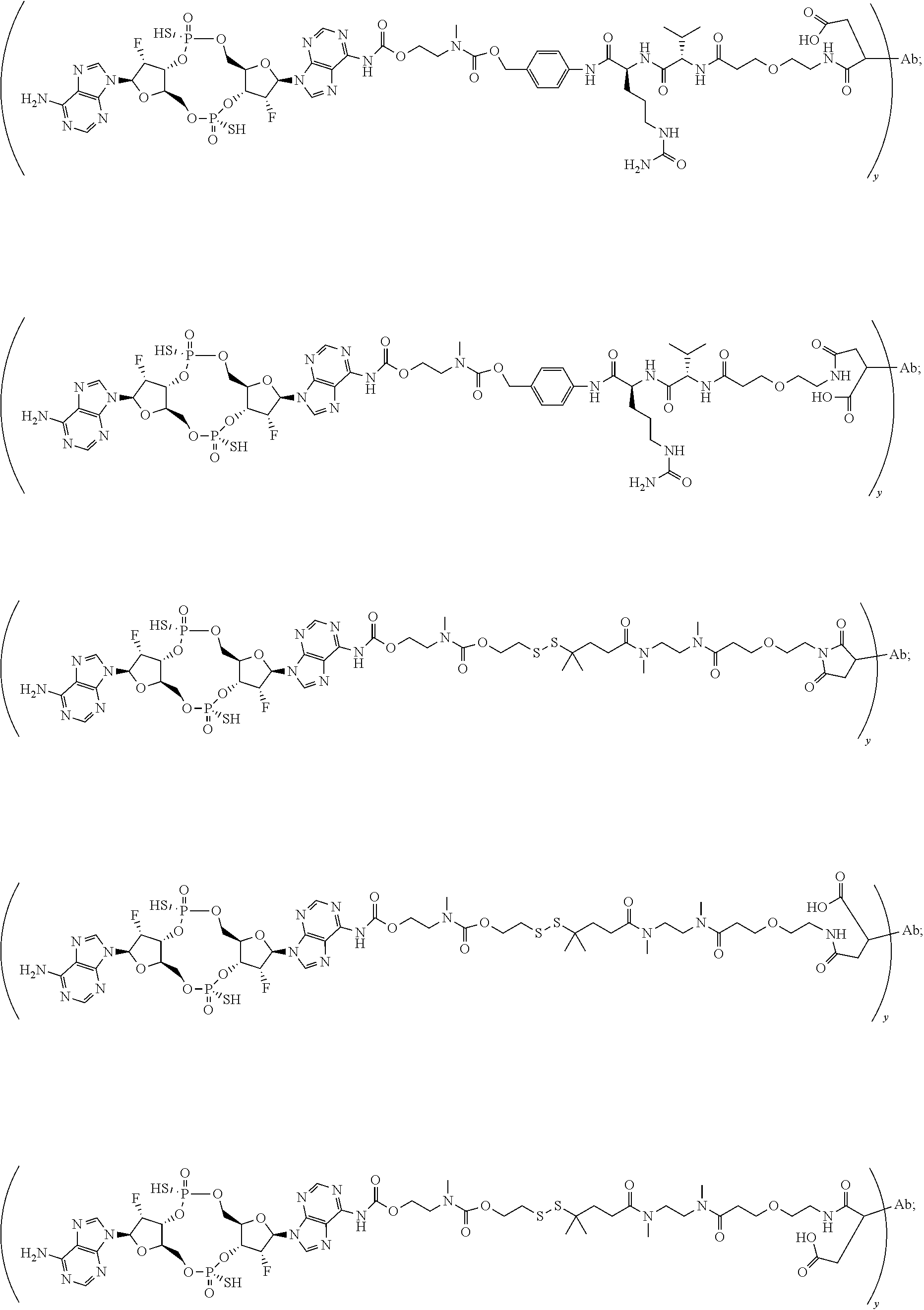

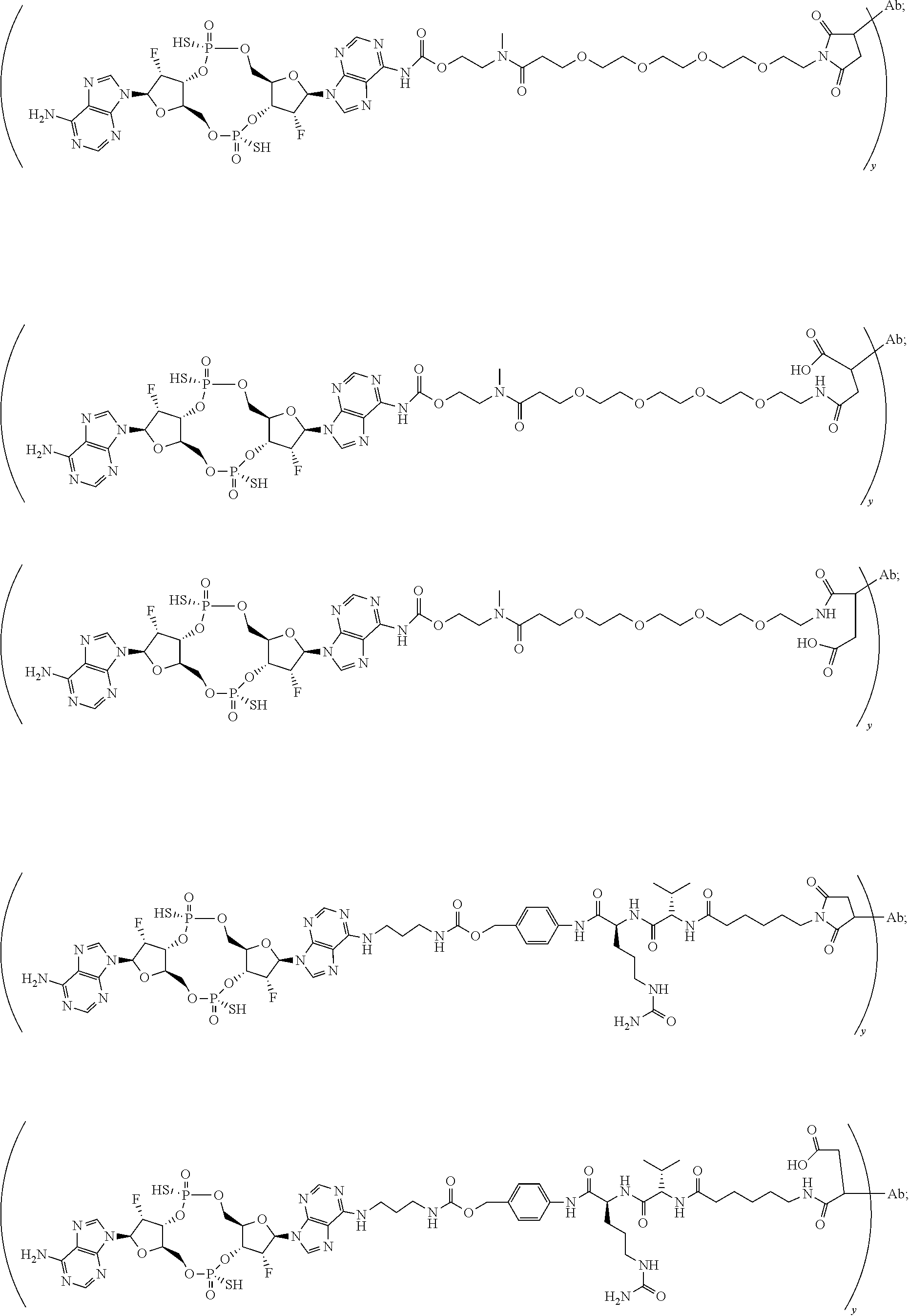

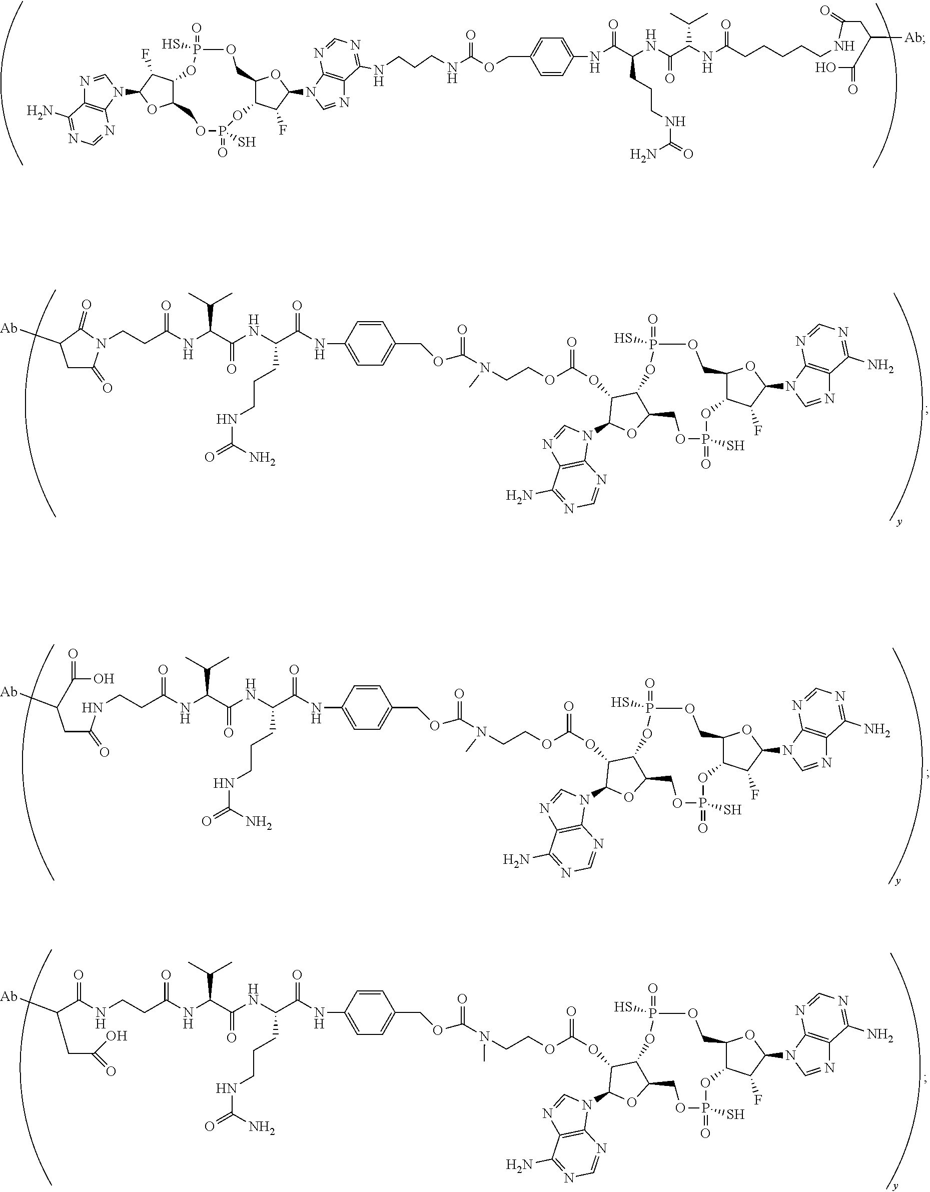

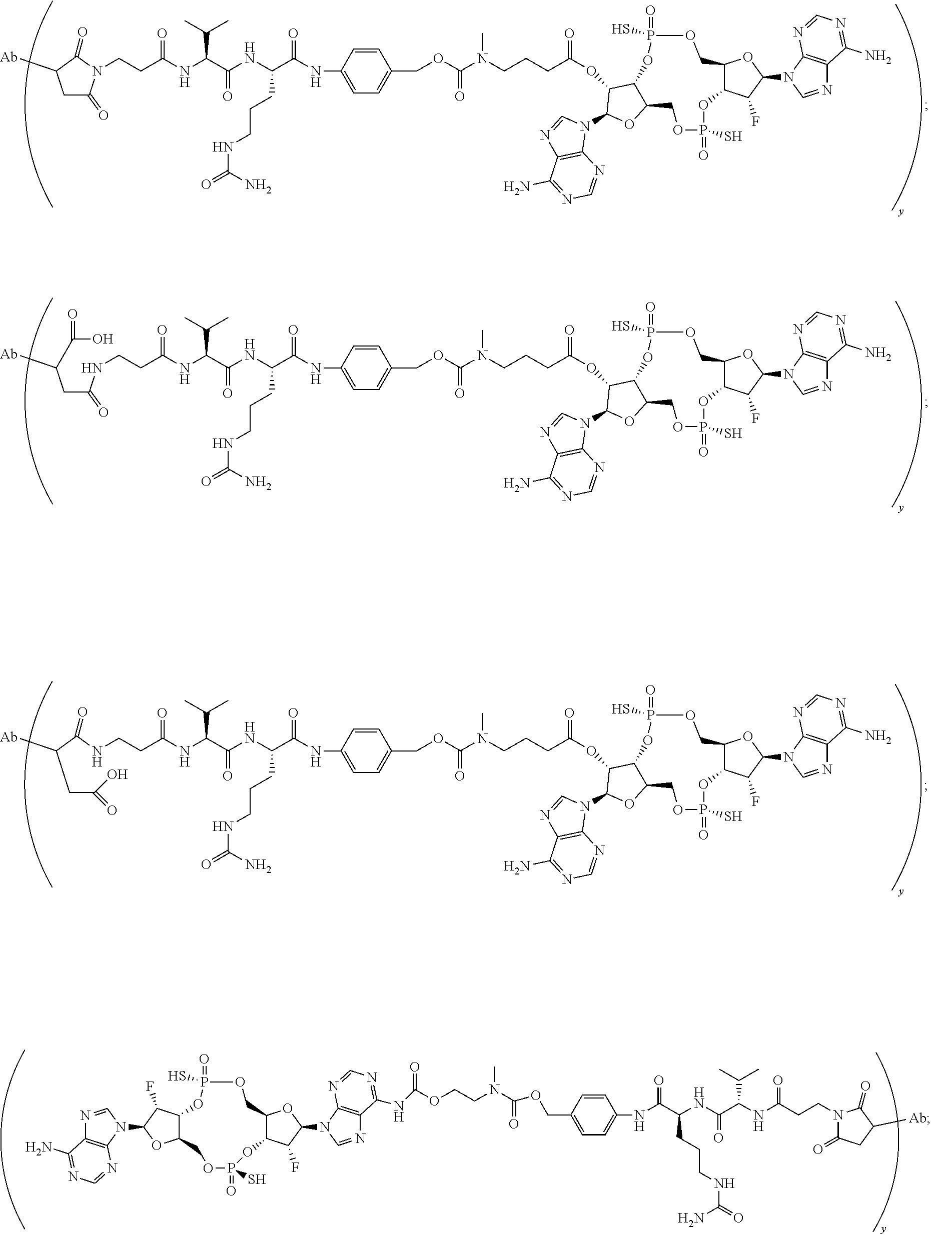

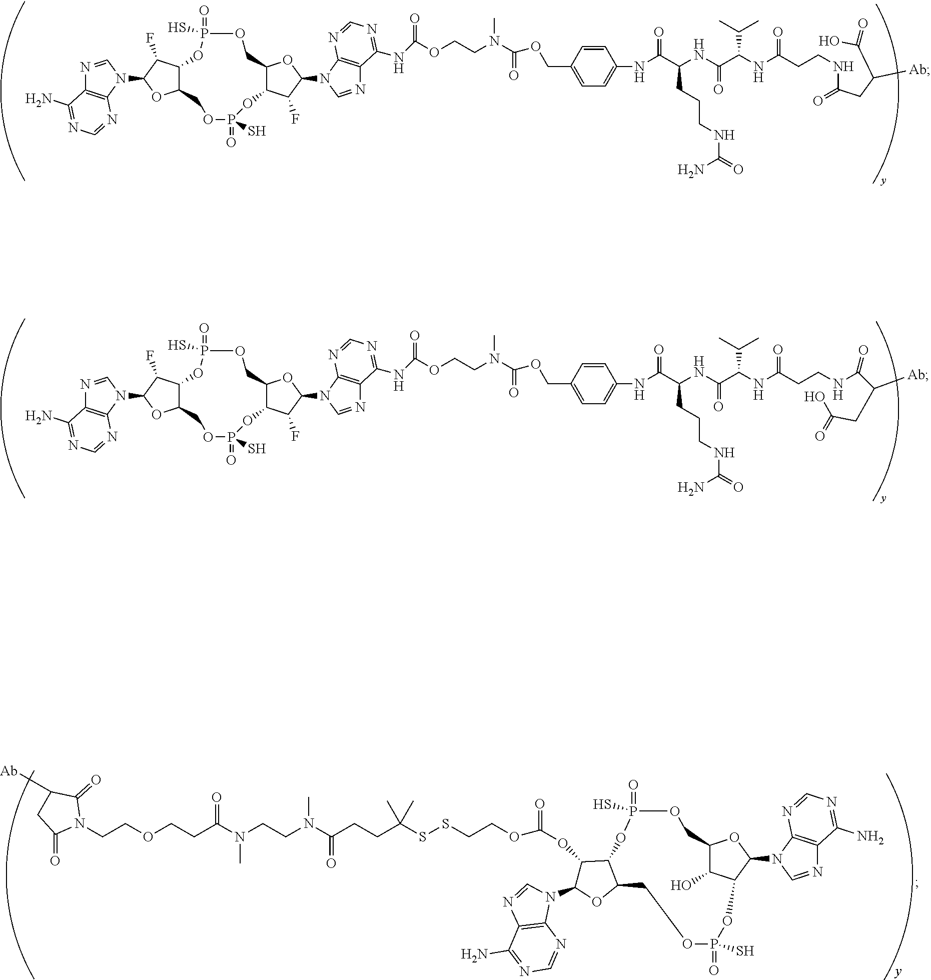

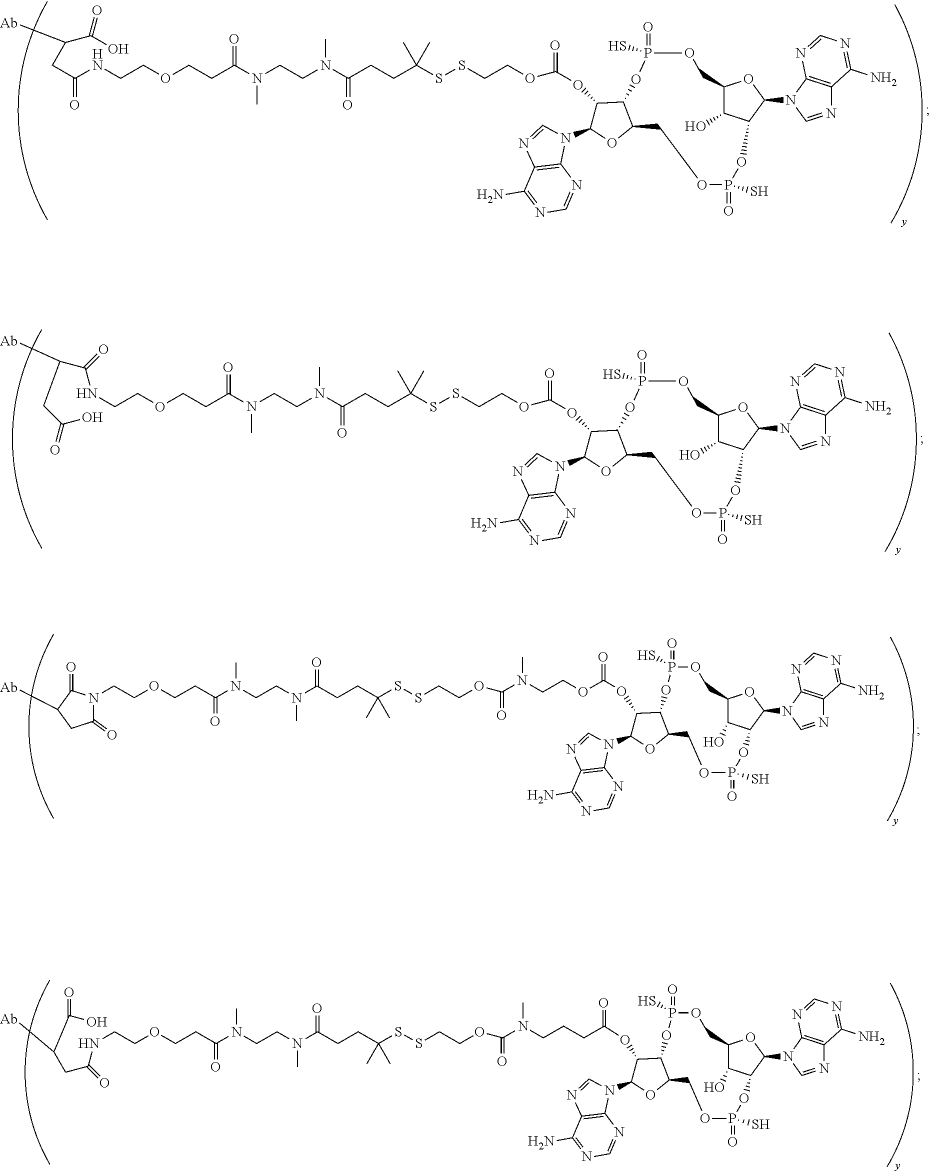

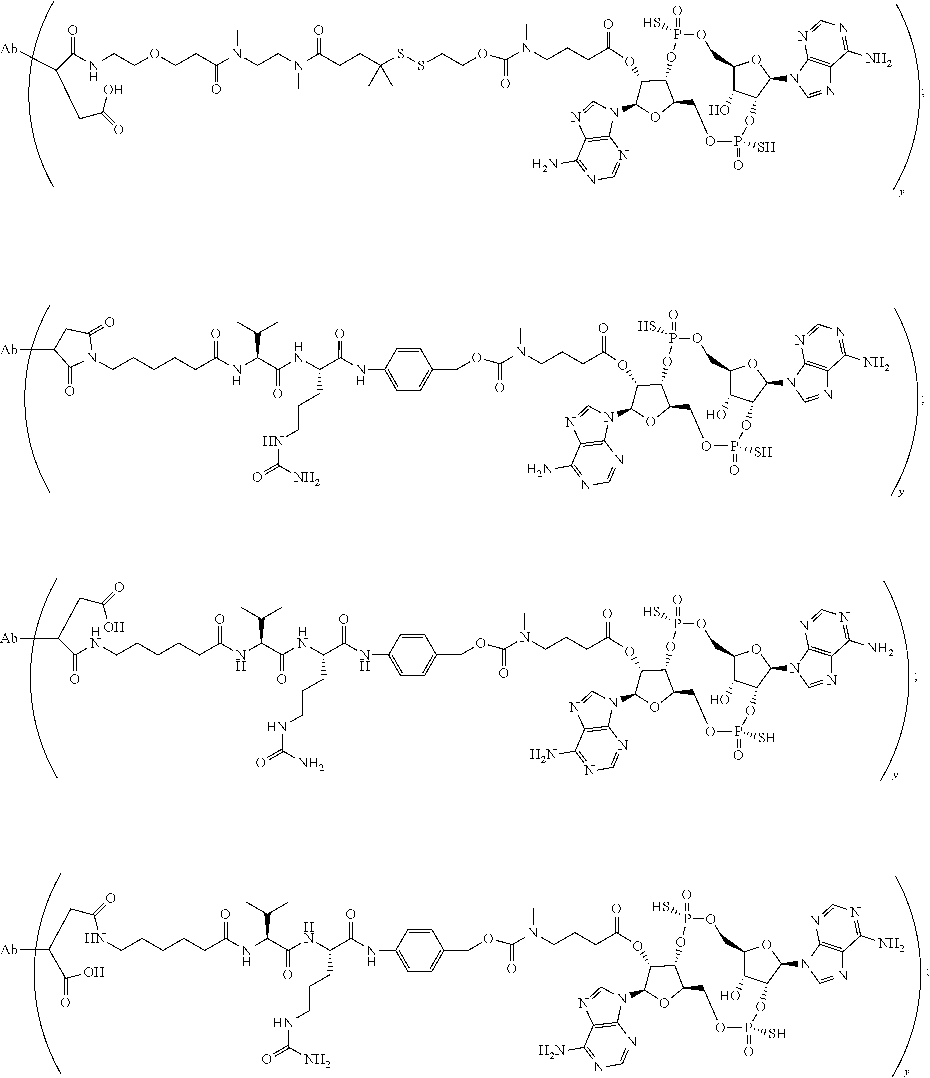

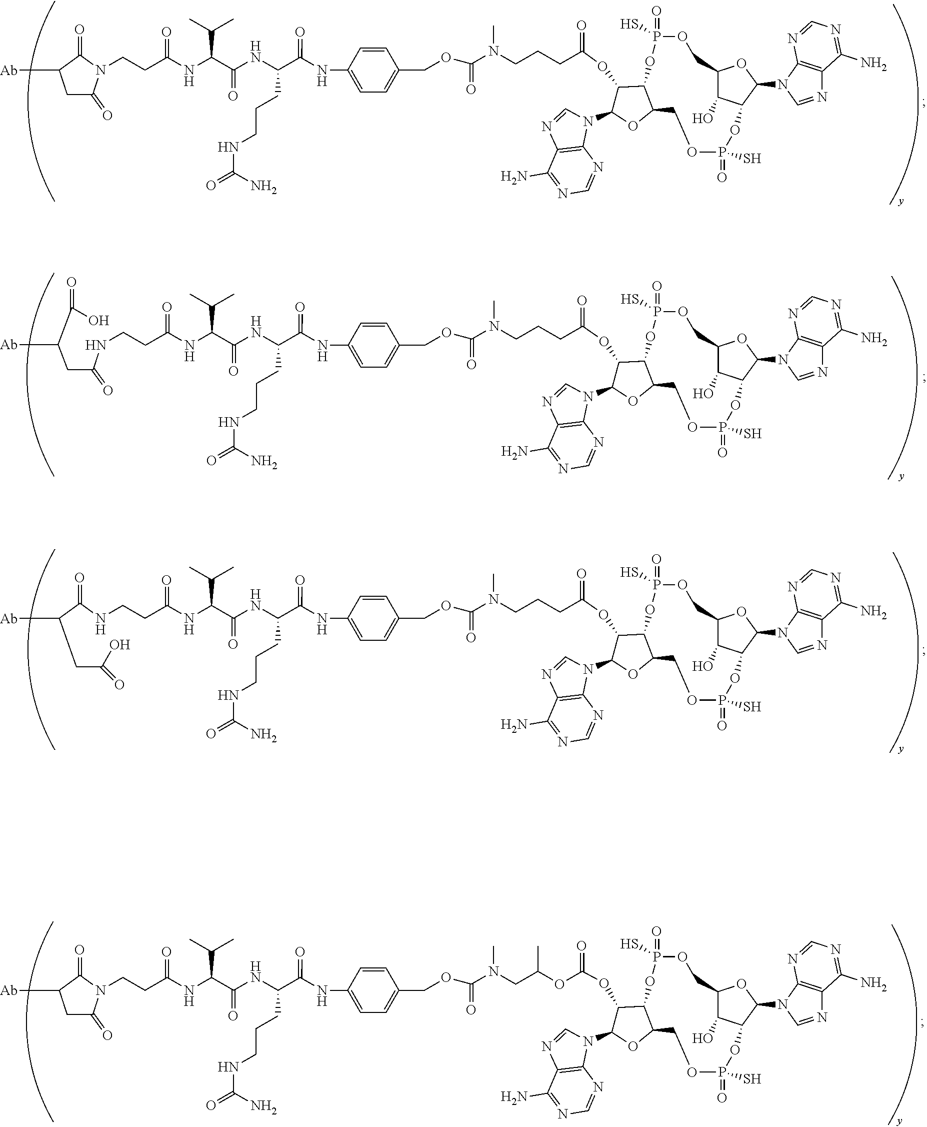

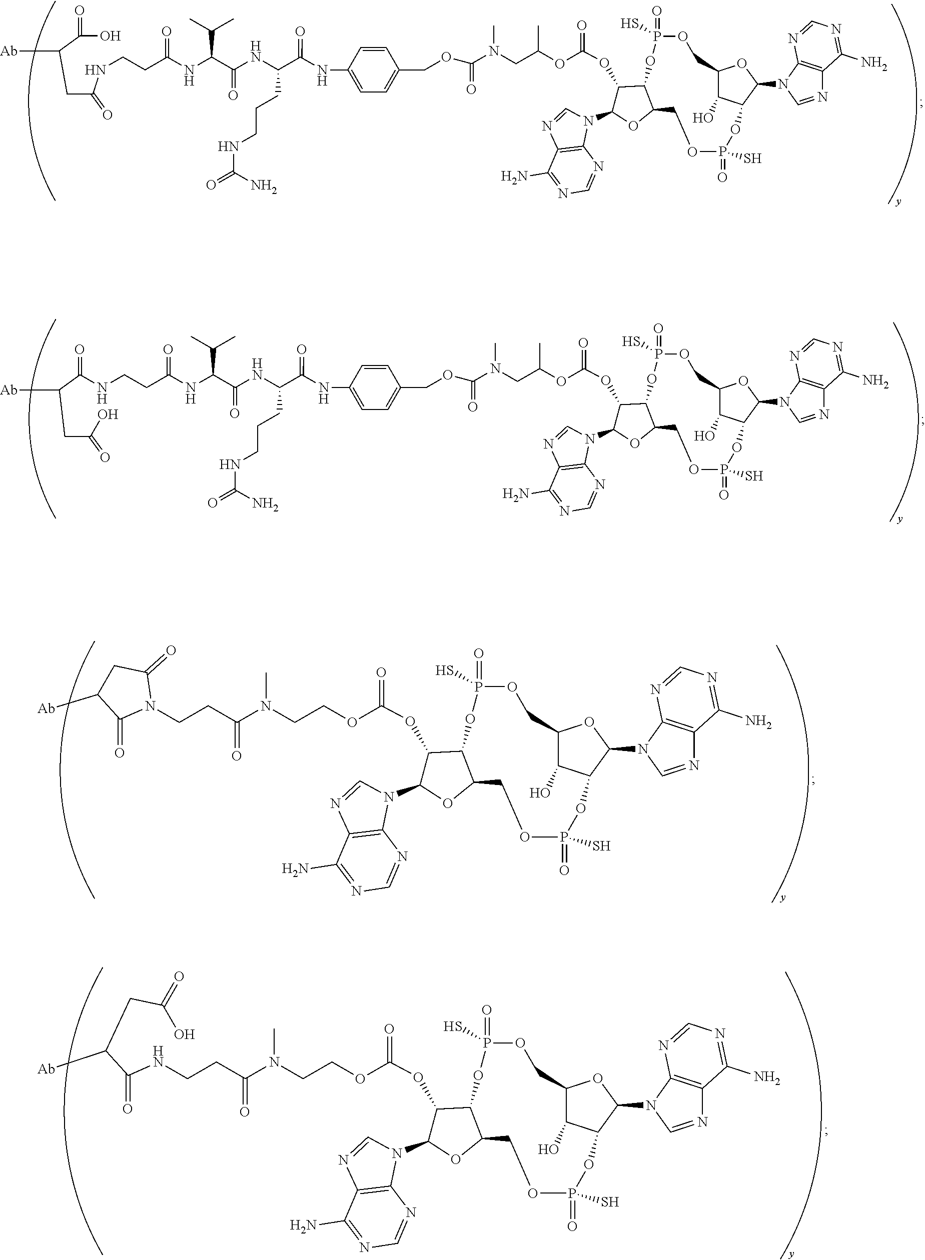

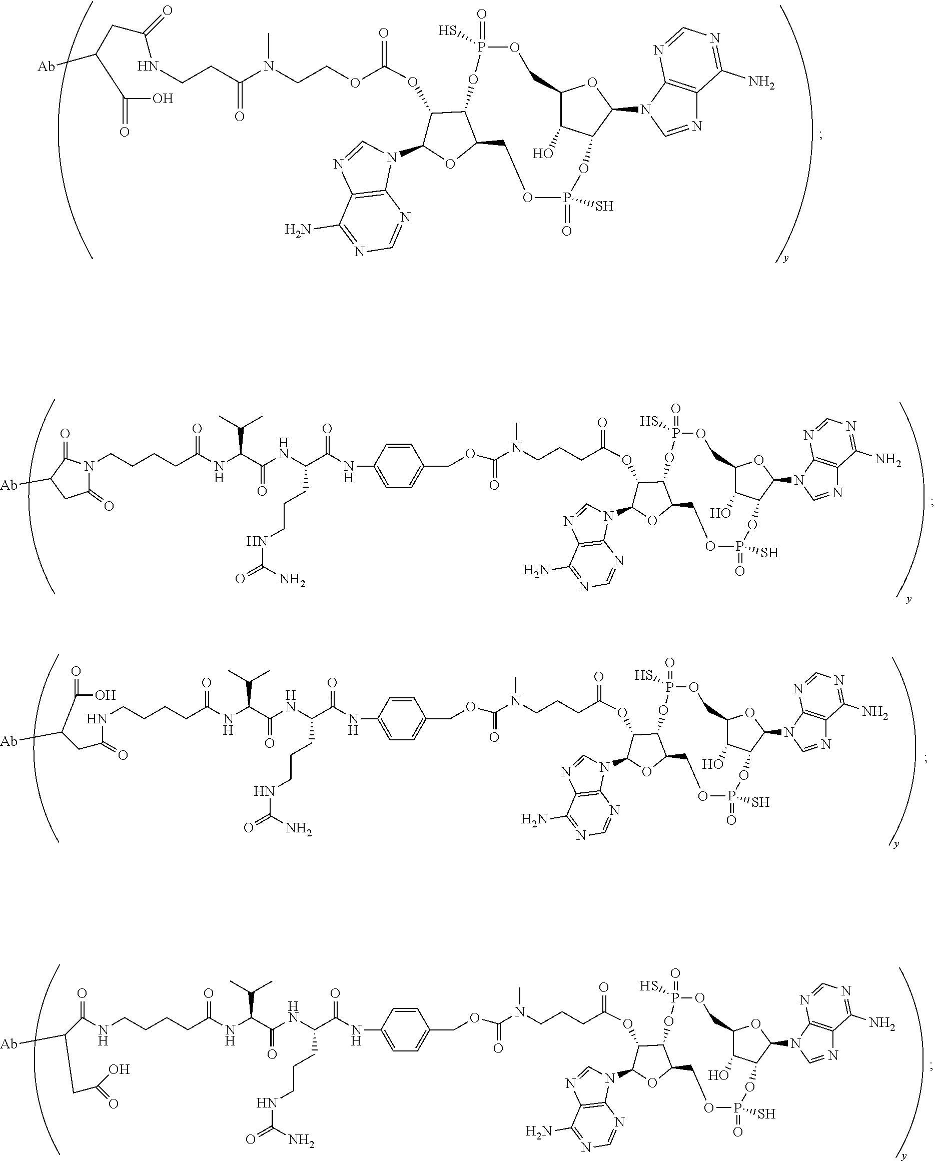

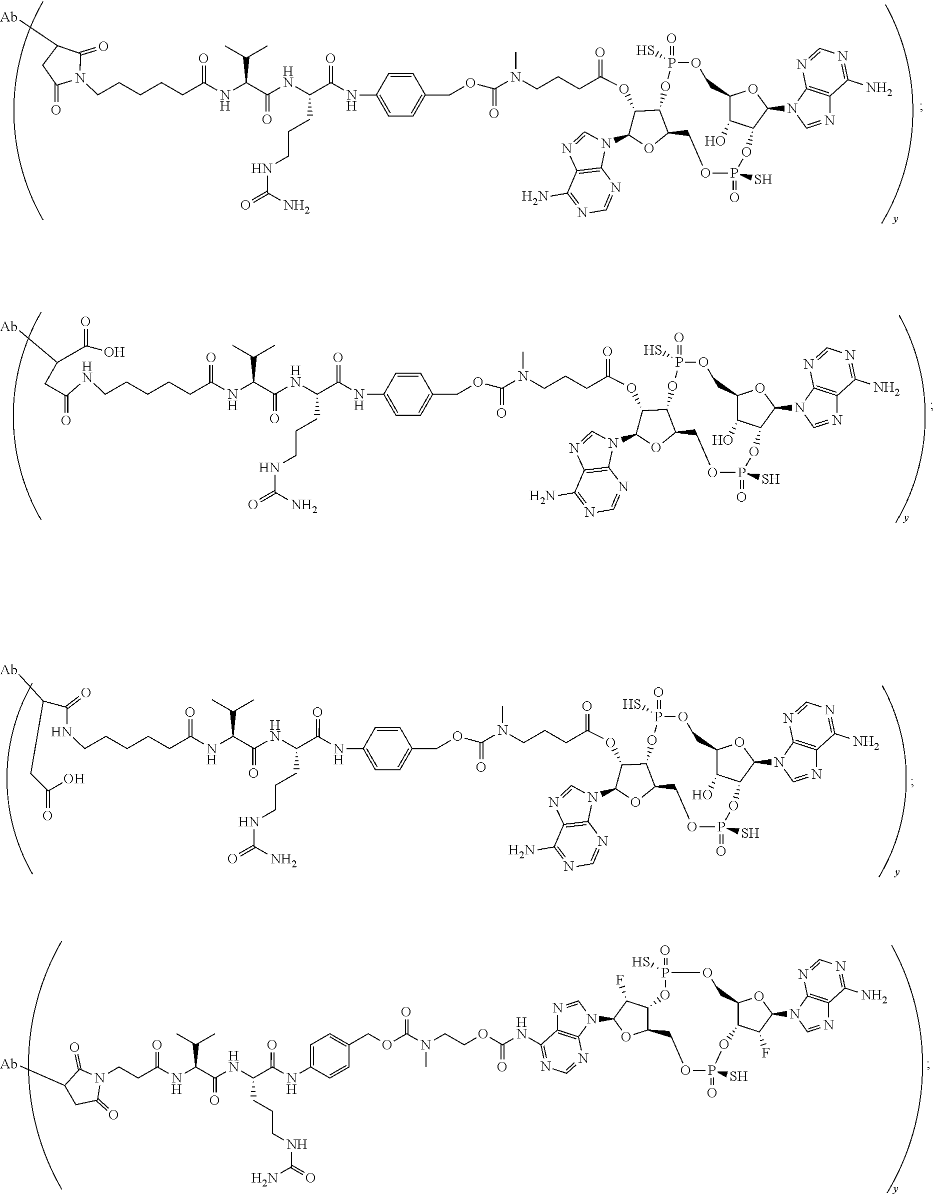

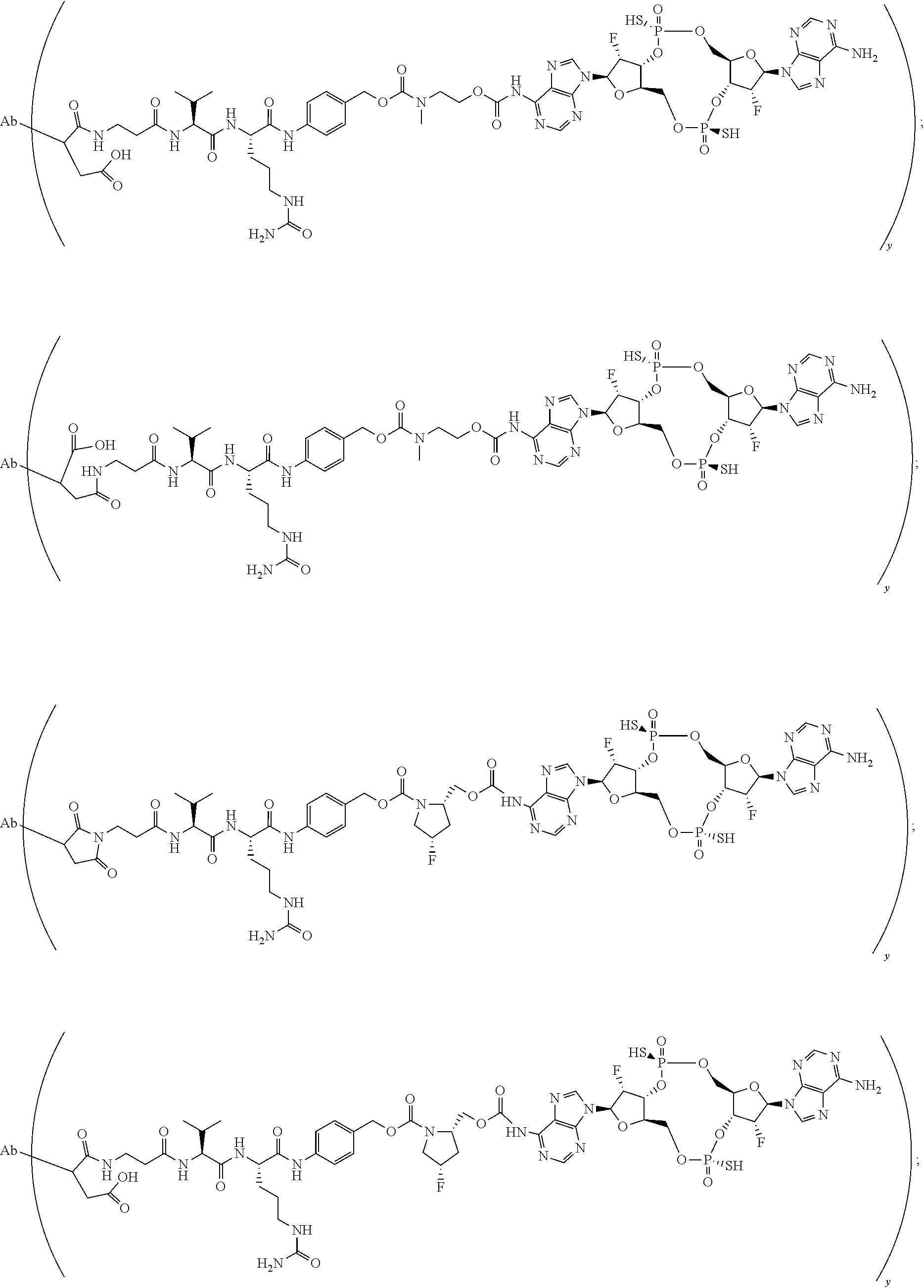

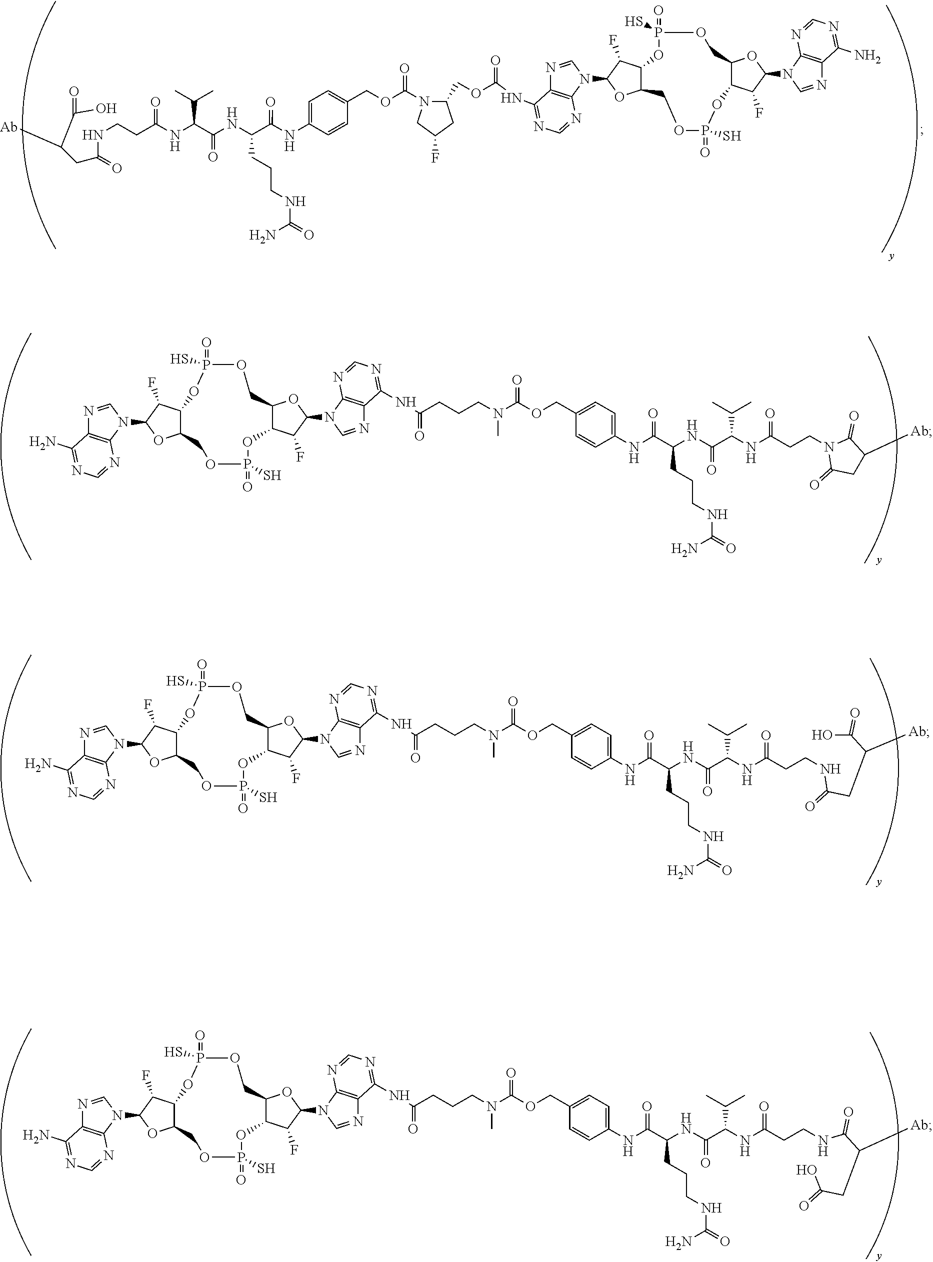

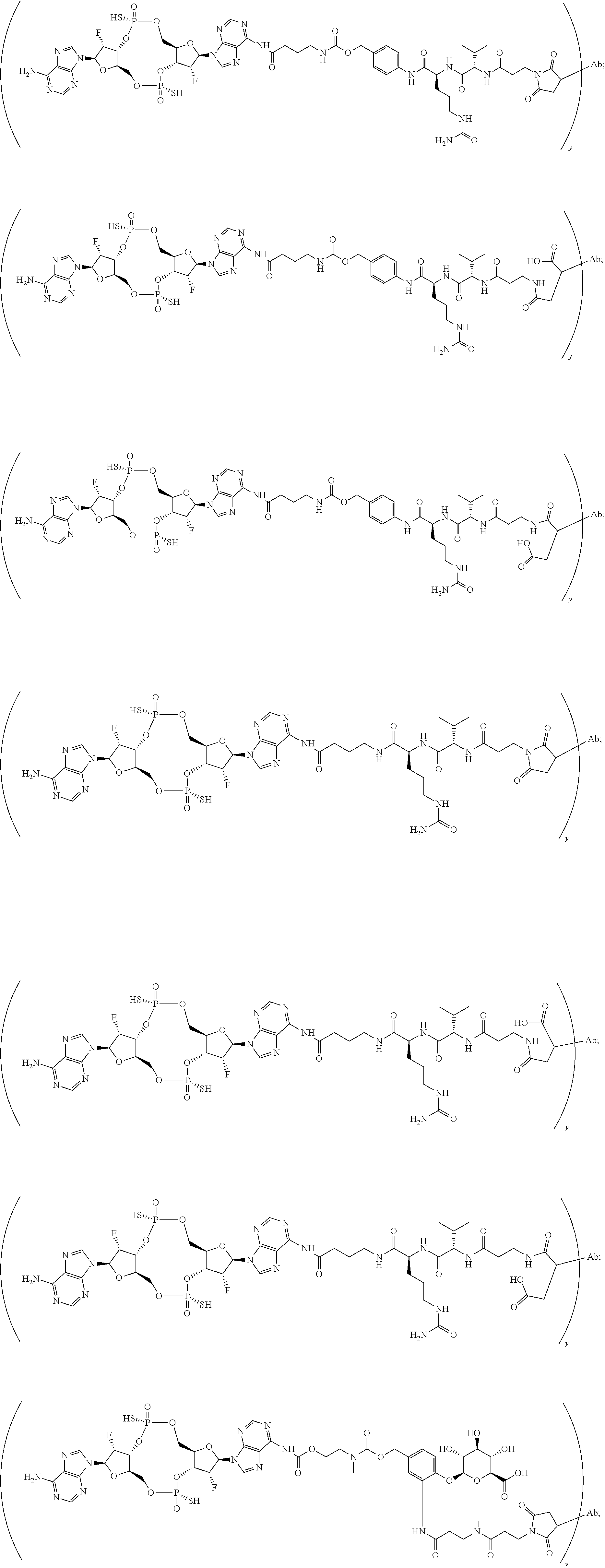

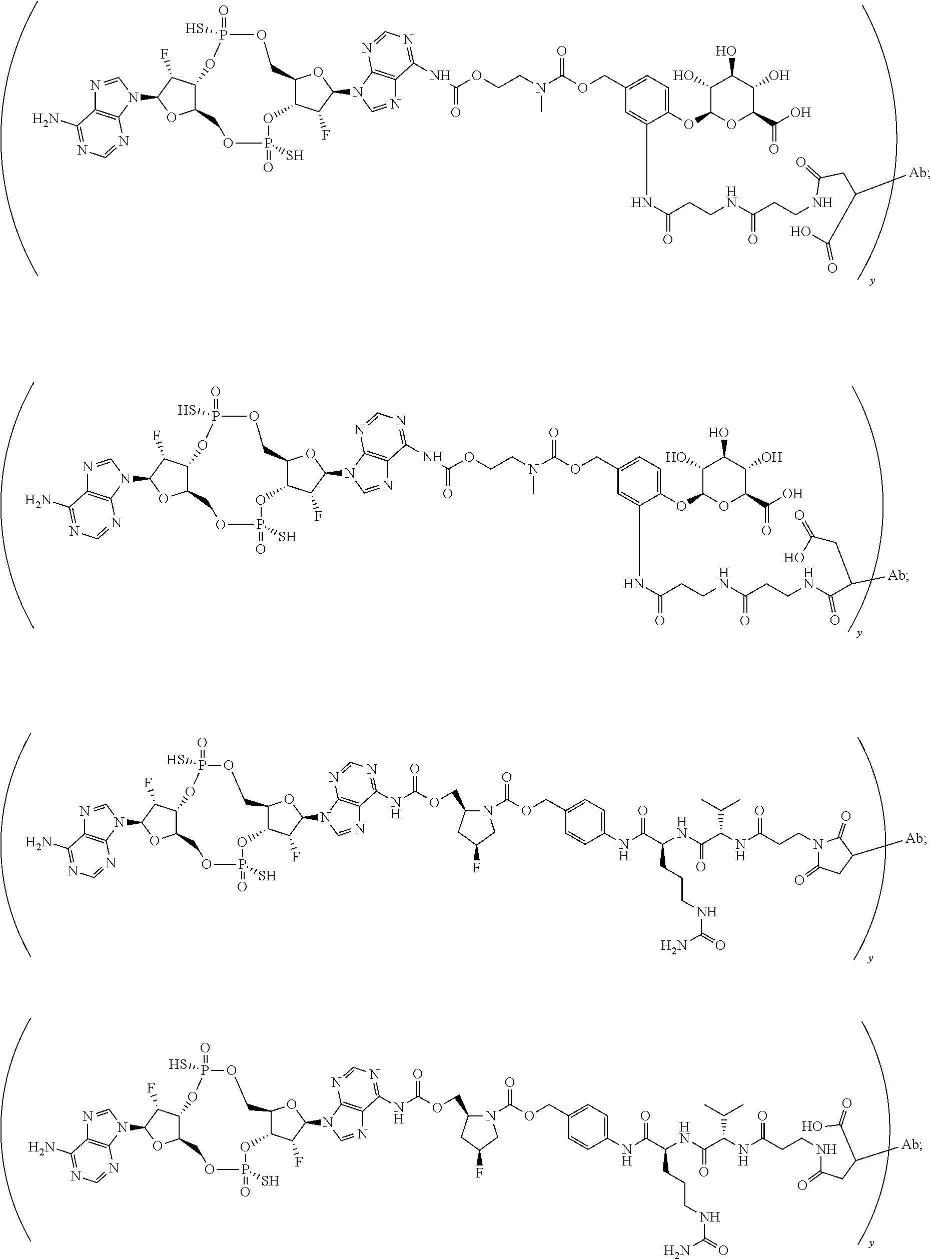

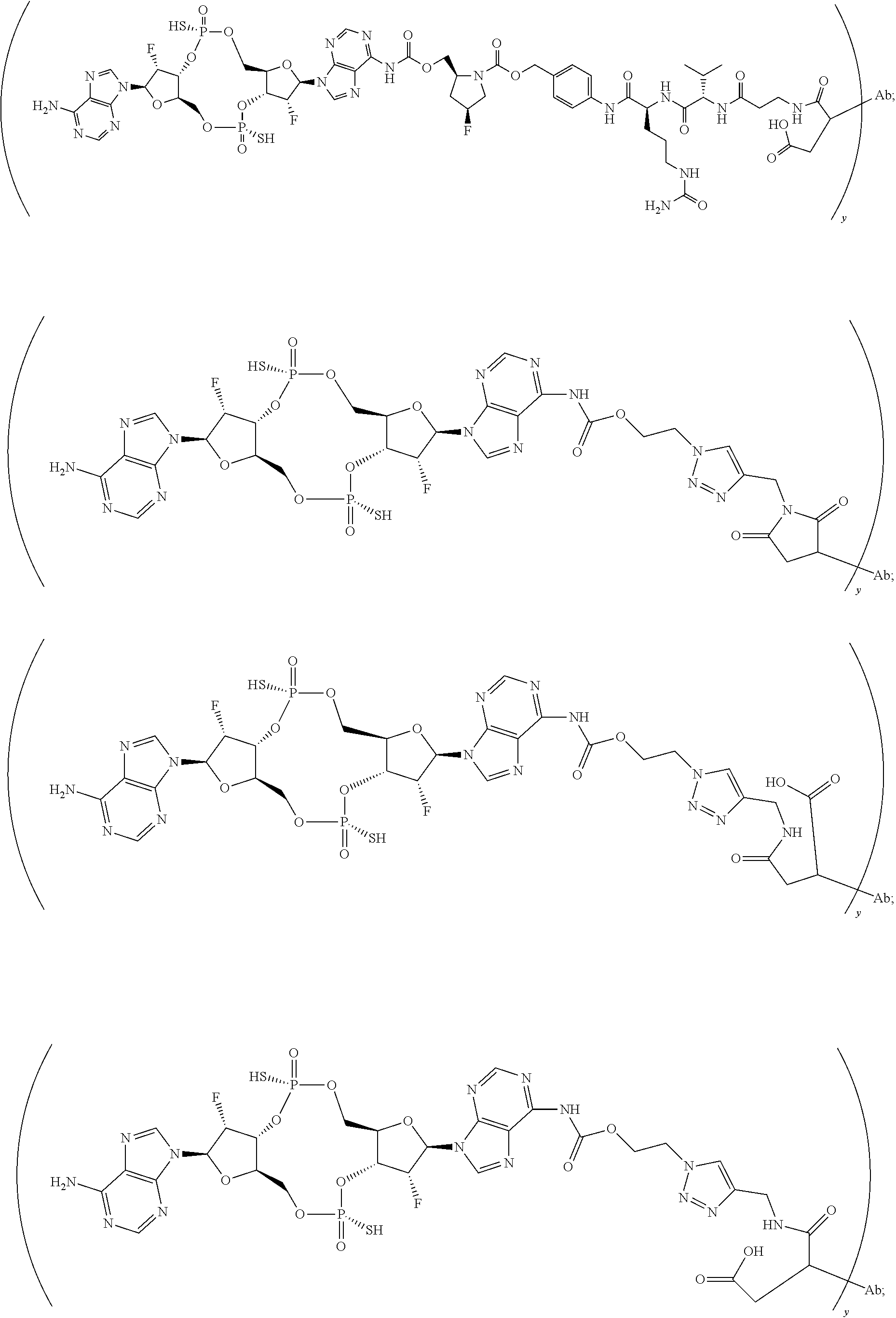

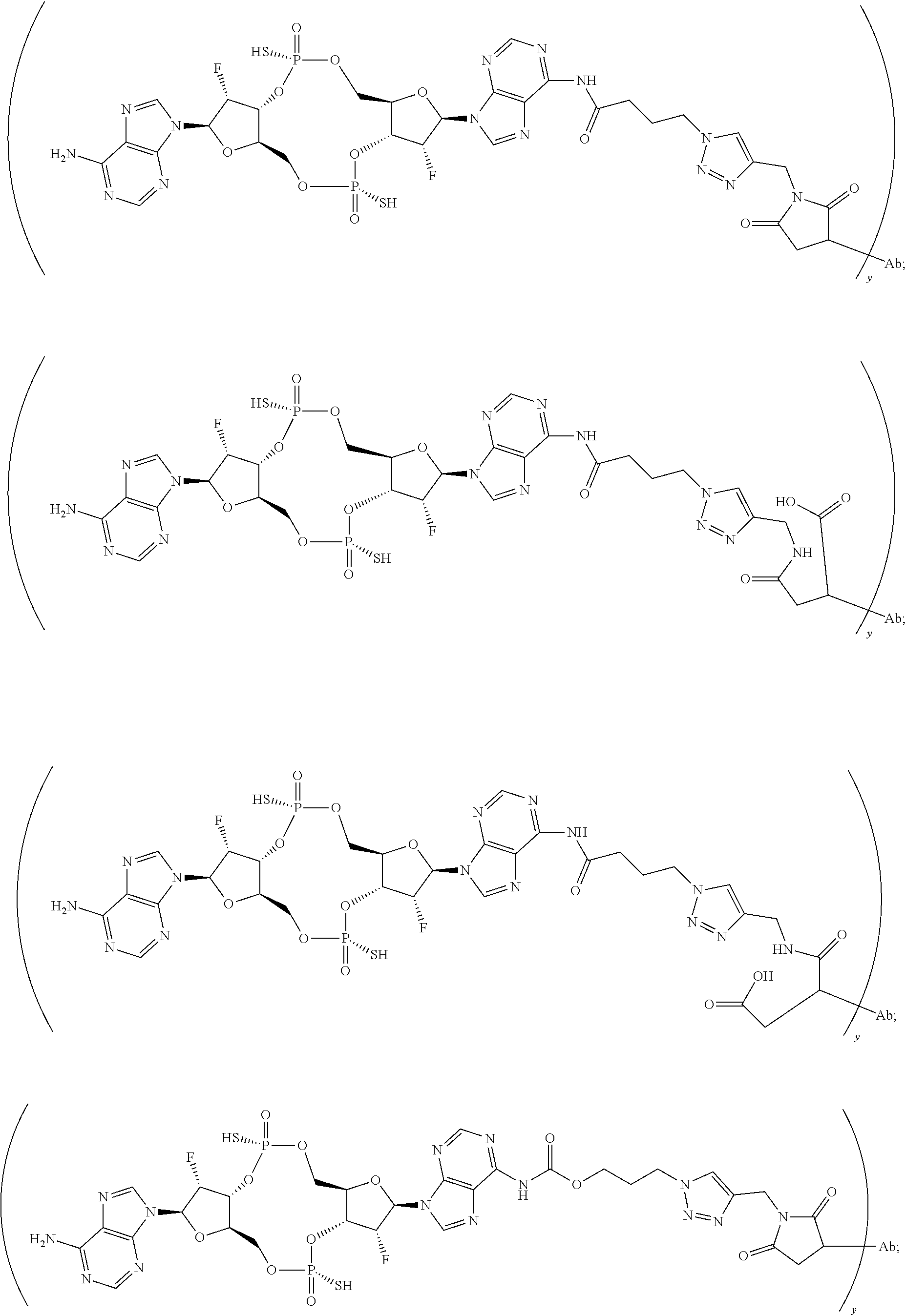

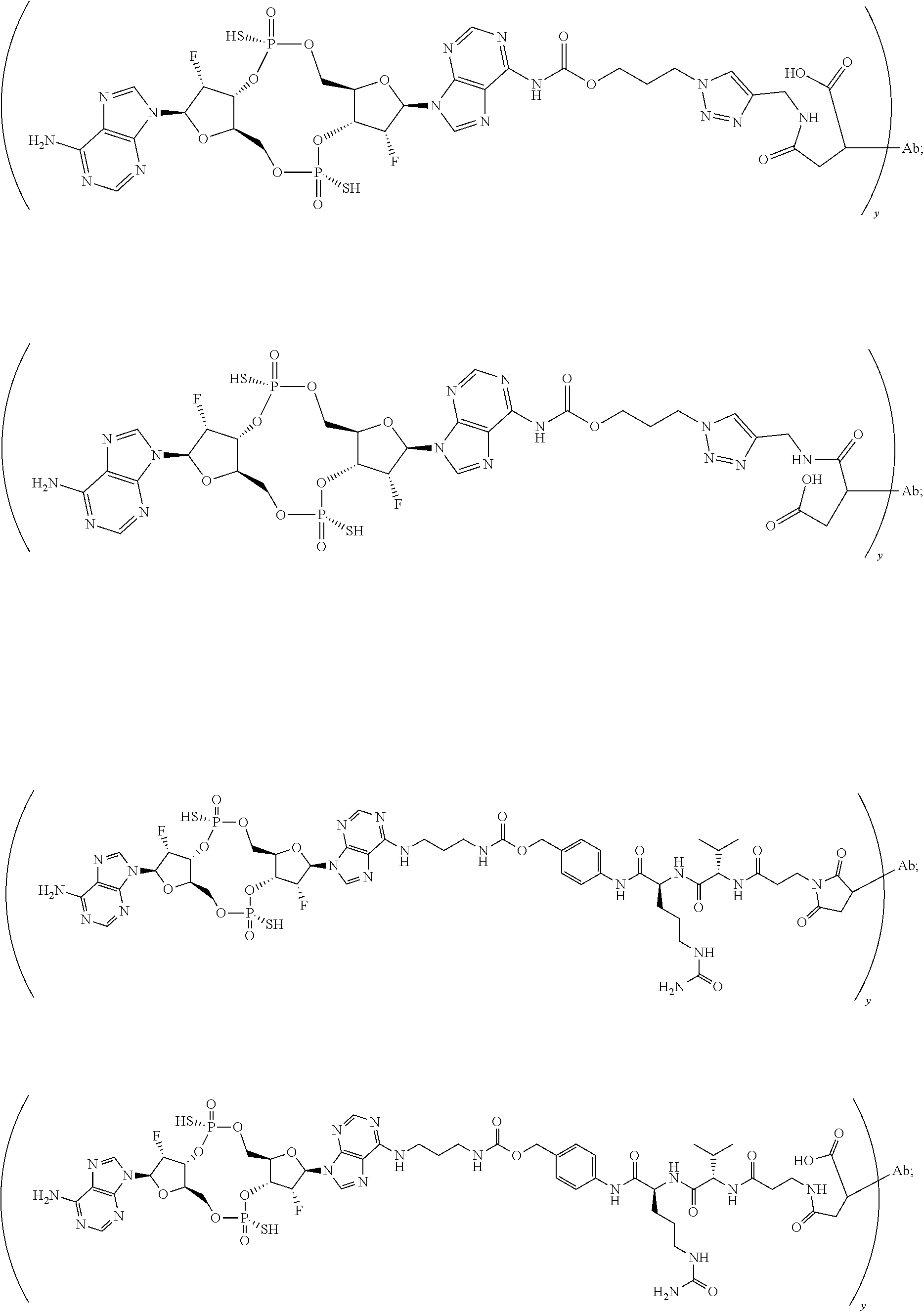

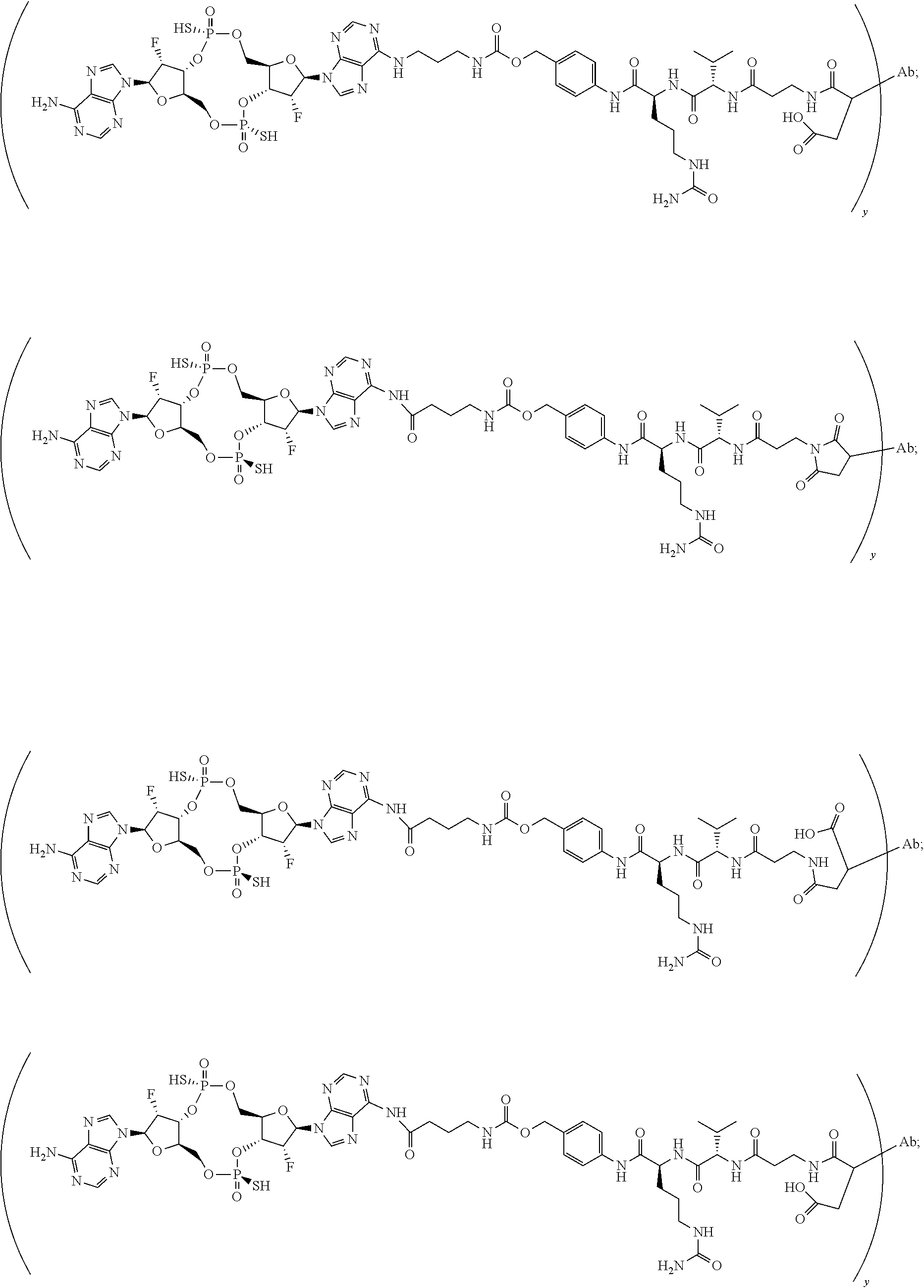

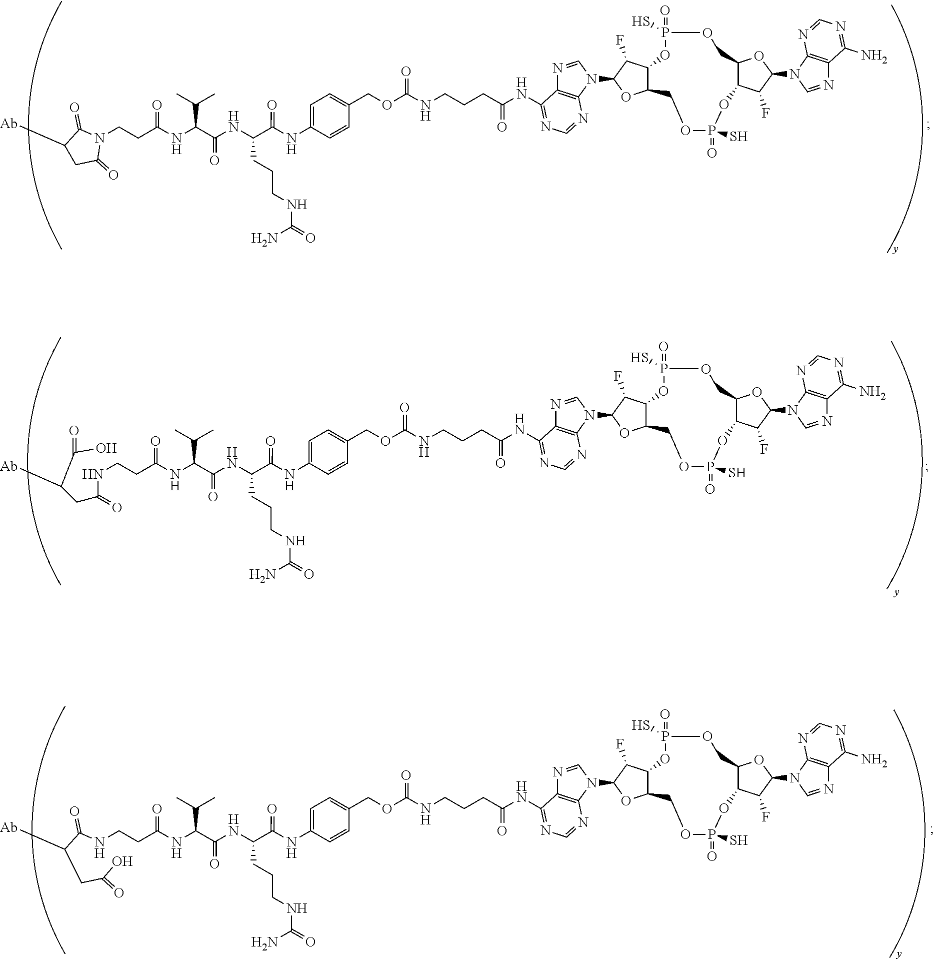

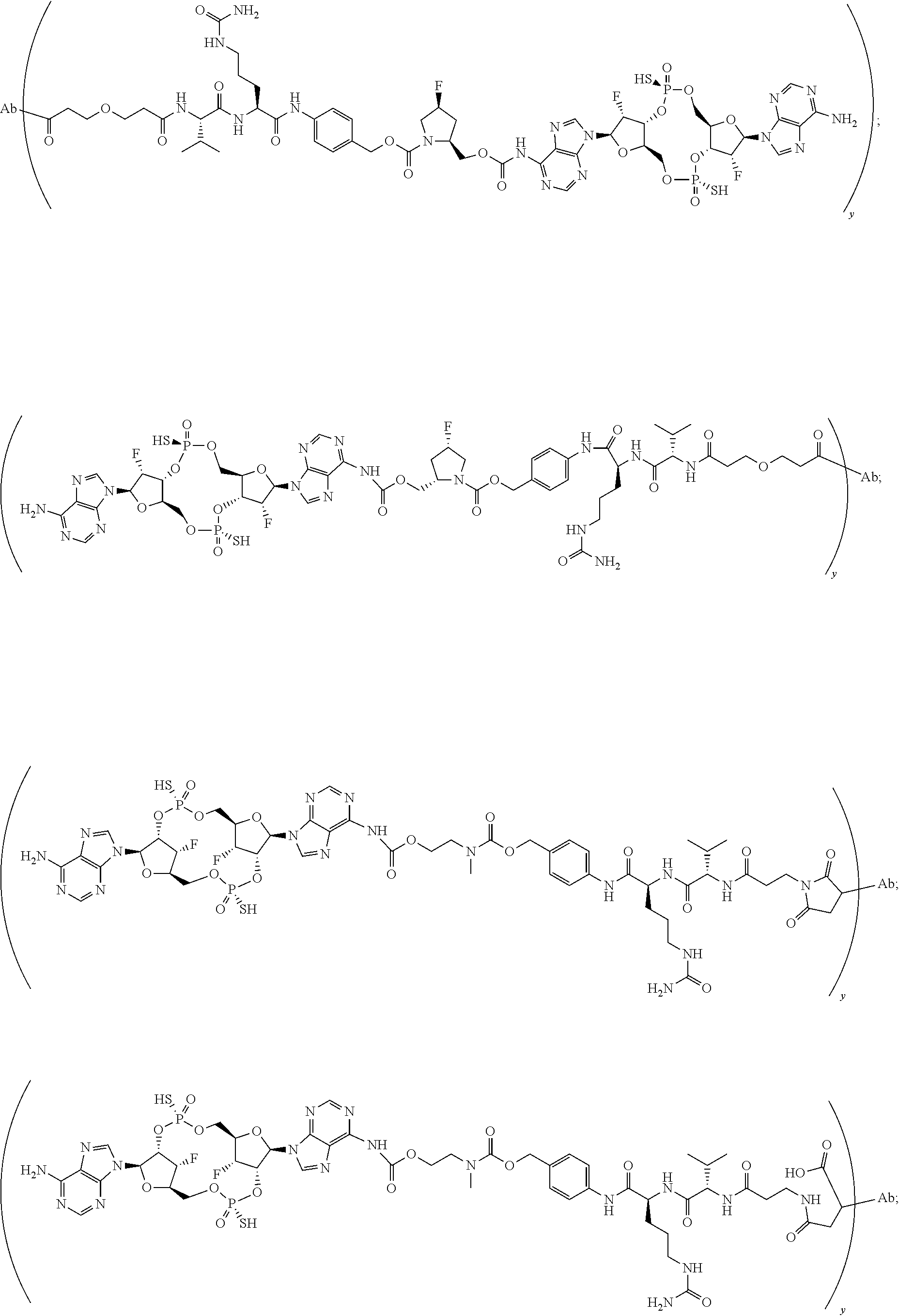

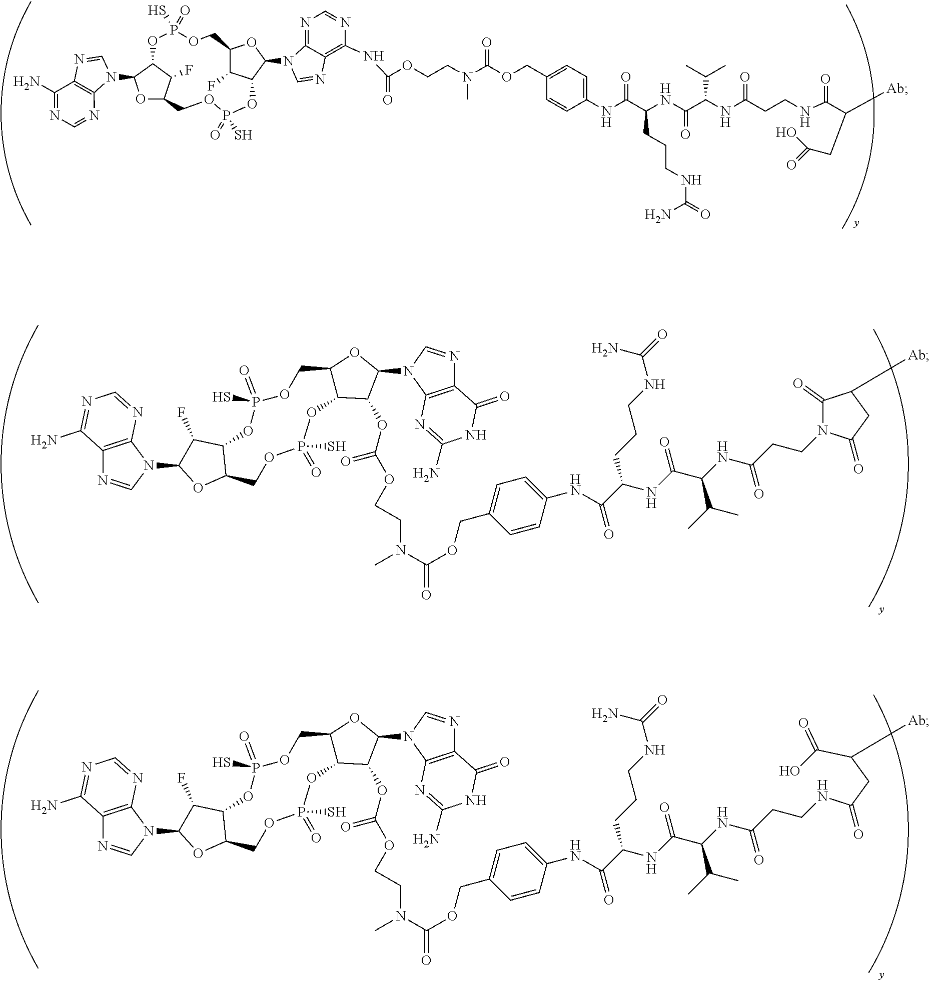

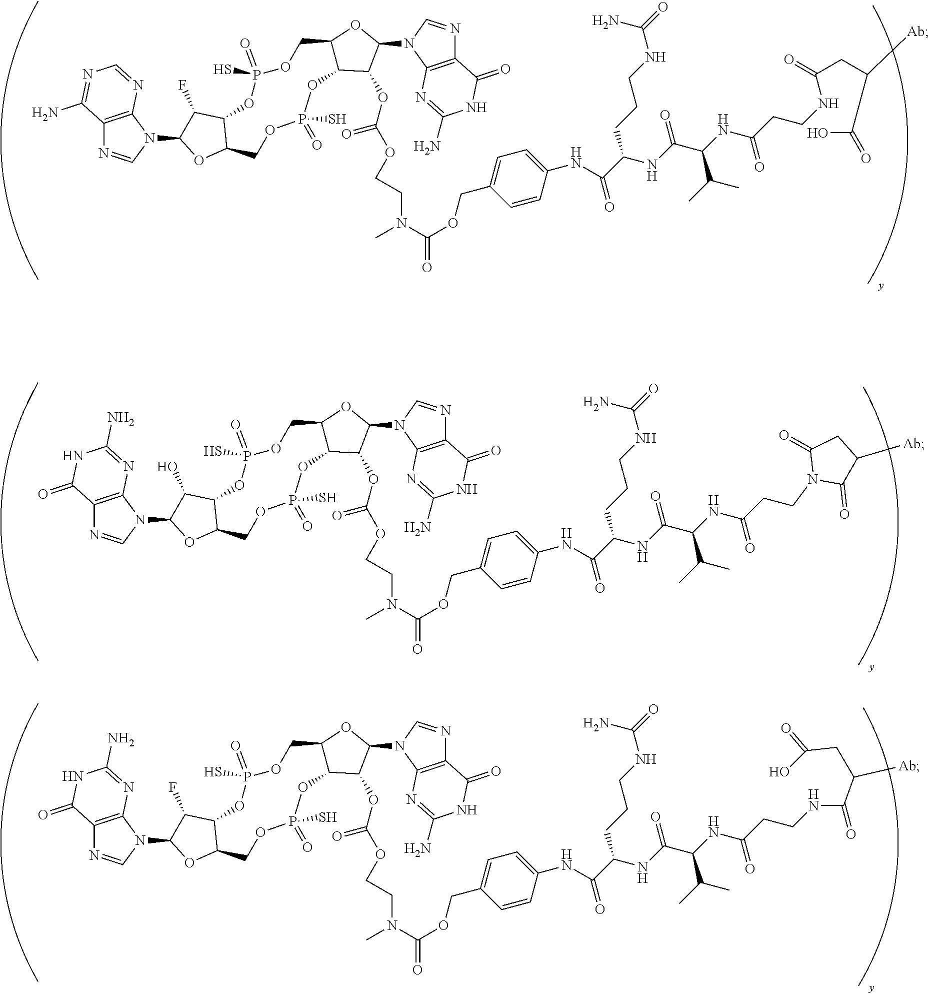

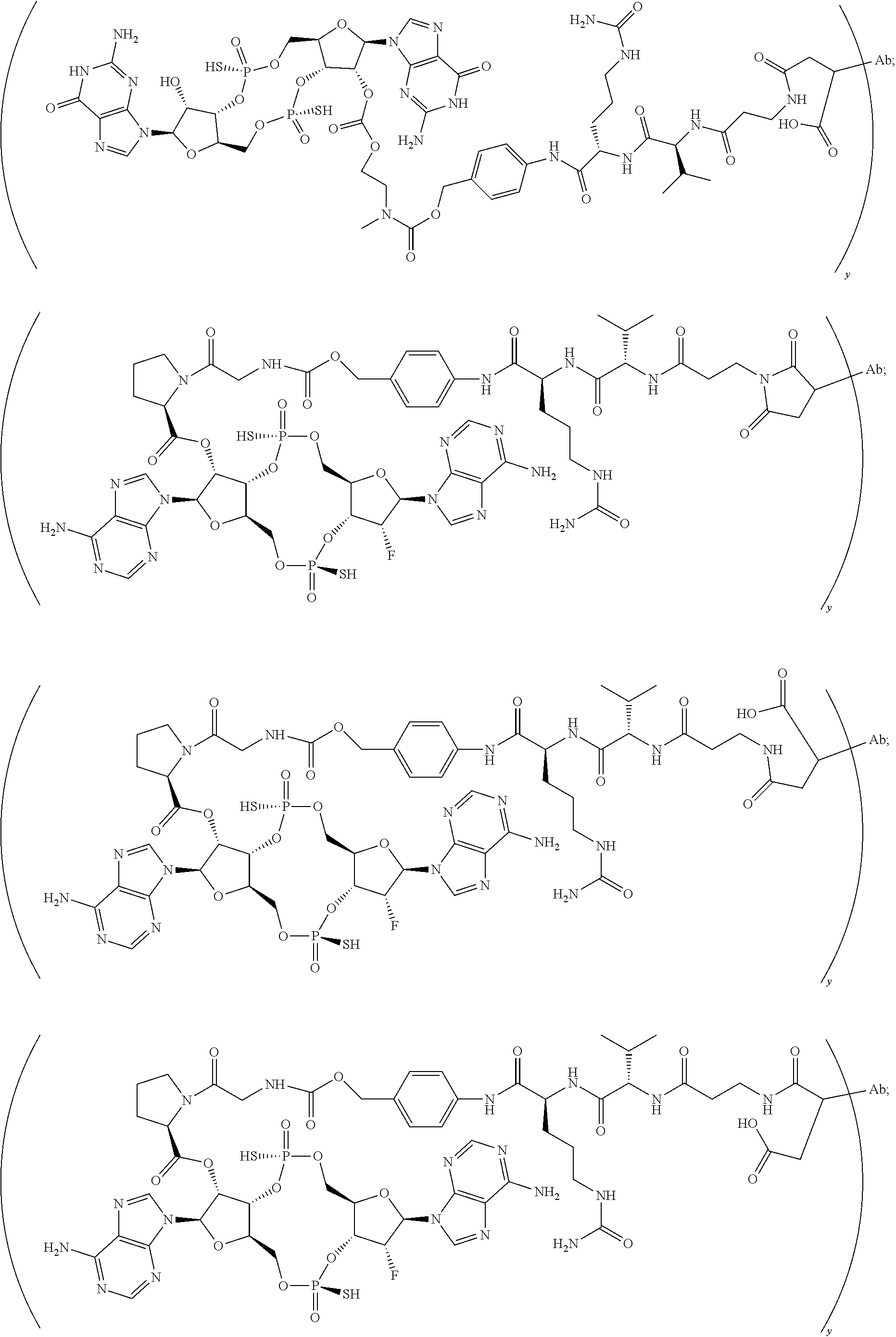

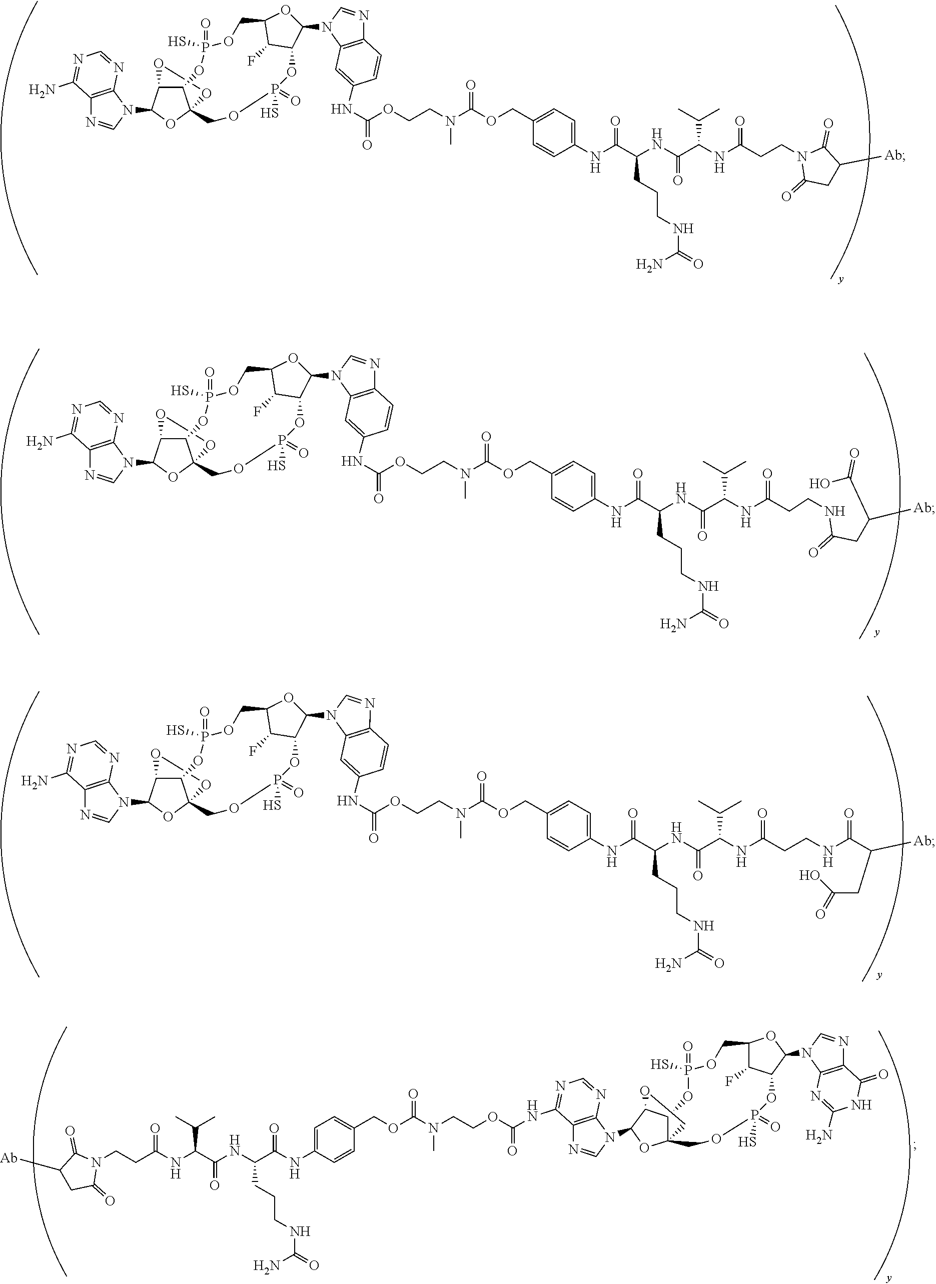

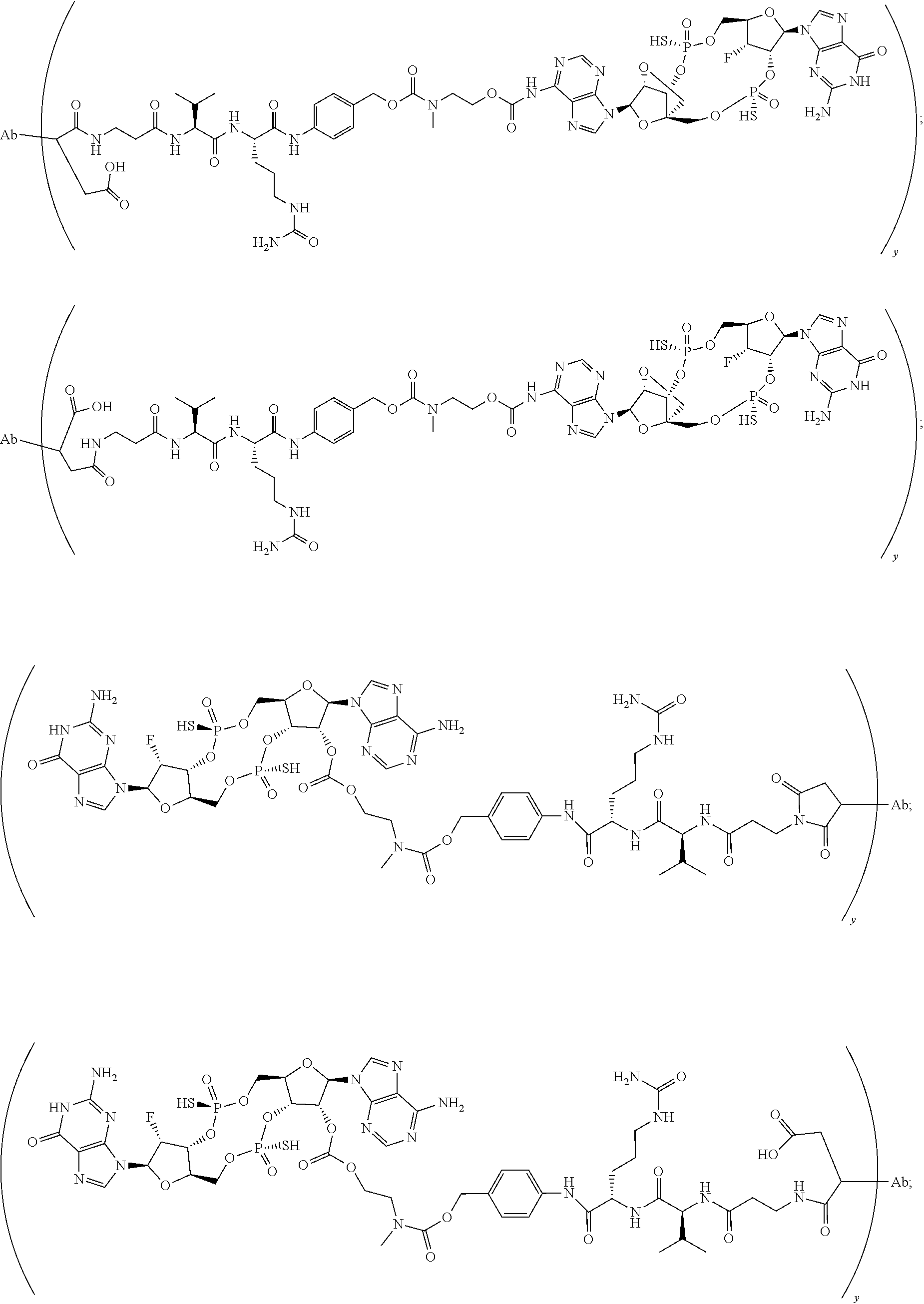

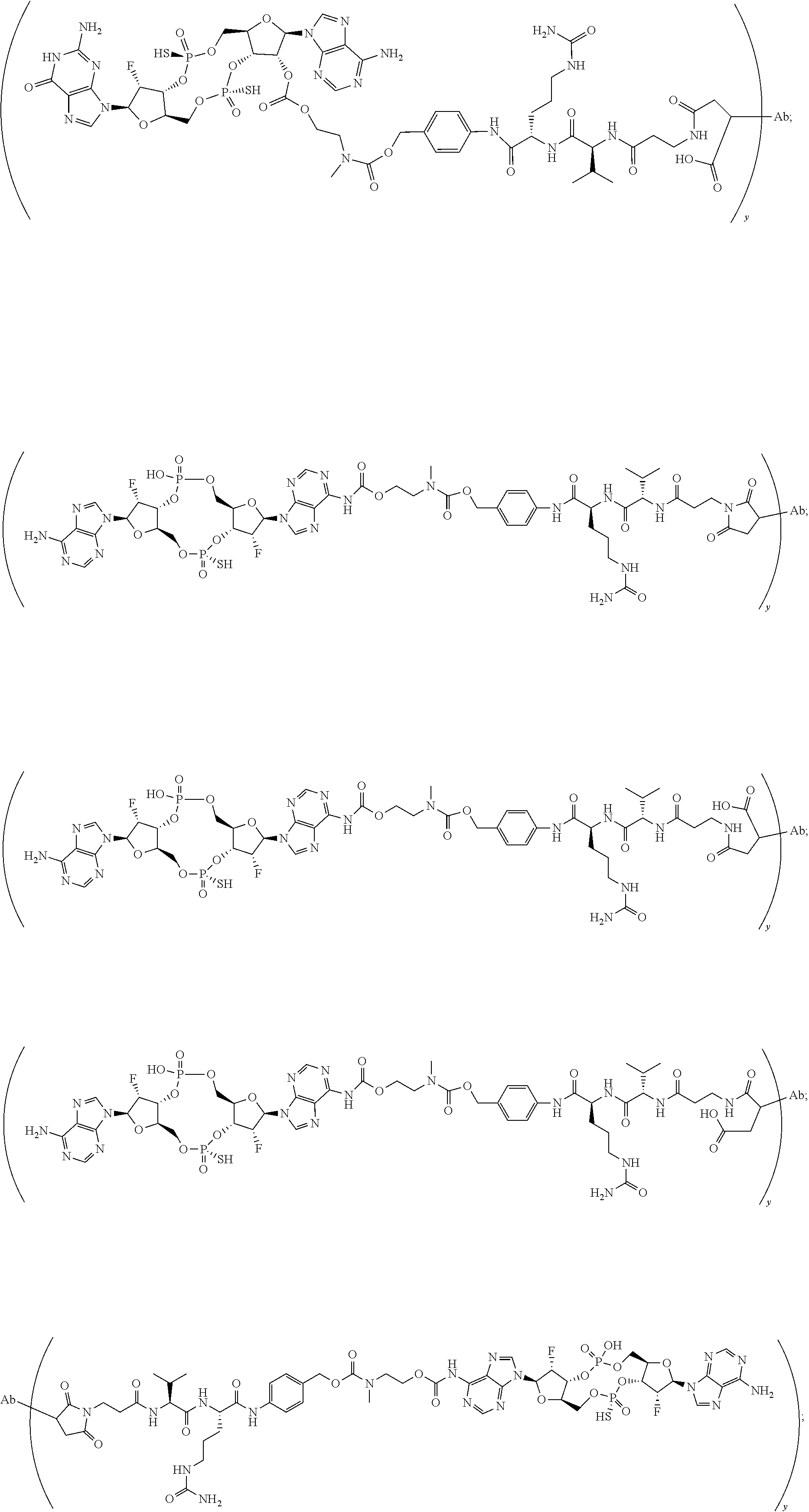

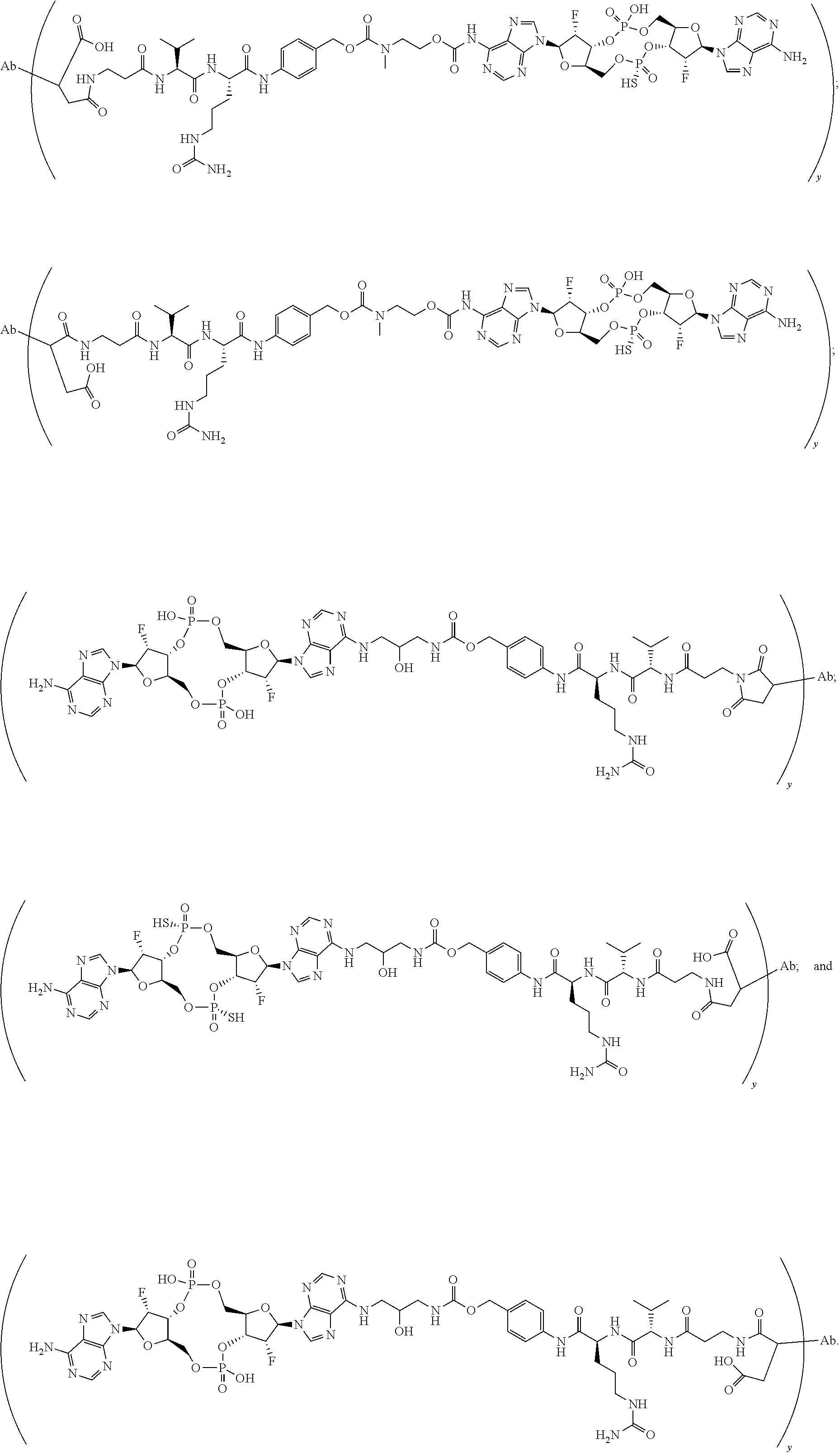

44. (canceled)

45. A pharmaceutical composition comprising the immunconjugate of claim 1 and a pharmaceutically acceptable excipient.

46. A composition comprising the immunoconjugate of claim 1 in combination and one or more additional therapeutic agents.

47. The composition of claim 46, wherein the additional therapeutic agent is selected from the group consisting of an inhibitor of a co-inhibitory molecule, an activator of a co-stimulatory molecule, a cytokine, an agent that reduces cytokine release syndrome (CRS), a chemotherapy, a targeted anti-cancer therapy, an oncolytic drug, a cytotoxic agent, an immune-based therapy, a vaccine, or a cell therapy.

48. The composition of claim 46, wherein the additional therapeutic agent is an inhibitor of a co-inhibitory molecule, an activator of a co-stimulatory molecule, or a cytokine, wherein:
(i) the co-inhibitory molecule is selected from Programmed death-1 (PD-1), Programmed death-ligand 1 (PD-L1), Lymphocyte activation gene-3 (LAG-3), or T-cell immunoglobulin domain and mucin domain 3 (TIM-3),
(ii) the co-stimulatory molecule is Glucocorticoid-induced TNFR-related protein (GITR), and
(iii) the cytokine is IL-15 complexed with a soluble form of IL-15 receptor alpha (IL-15Ra).

49. A method of treating cancer comprising administering to a patient in need thereof a therapeutically effective amount of the immunconjugate of claim 1.

50-52. (canceled)

53. The method according to claim 49, wherein the cancer is selected from sarcomas, adenocarcinomas, blastomas, carcinomas, liver cancer, lung cancer, non-small cell lung cancer, small cell lung cancer, breast cancer, lymphoid cancer, colon cancer, renal cancer, urothelial cancer, prostate cancer, cancer of the pharynx, rectal cancer, renal cell carcinoma, cancer of the small intestine, esophageal cancer, melanoma, bone cancer, pancreatic cancer, skin cancer, cancer of the head or neck, cutaneous or intraocular malignant melanoma, uterine cancer, ovarian cancer, colorectal cancer, cancer of the anal region, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, environmentally induced cancers including those induced by asbestos, leukemia, lymphoma, acute myelogenous leukemia (AML), acute lymphoid leukemia (ALL), chronic myelogenous leukemia (CML), chronic lymphoid leukemia (CLL), myelodysplastic syndromes, B-cell acute lymphoid leukemia (“BALL”), T-cell acute lymphoid leukemia (“TALL”), B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia, myelodysplastic syndrome, plasmablastic lymphoma, plasmacytoid dendritic cell neoplasm, and Waldenstrom macroglobulinemia.

54-56. (canceled)


SEQUENCE LISTING (941 sequences)

SEQ ID NO: 1 - protein, 10 aa, Artificial Sequence
  Description of Artificial Sequence: Synthetic peptide
GYTFTNYGIN

SEQ ID NO: 2 - protein, 17 aa, Artificial Sequence
  Description of Artificial Sequence: Synthetic peptide
YIYIGNDYTEYNERFKG

SEQ ID NO: 3 - protein, 14 aa, Artificial Sequence
  Description of Artificial Sequence: Synthetic peptide
LYYGSSLYSYAMDY

SEQ ID NO: 4 - protein, 5 aa, Artificial Sequence
  Description of Artificial Sequence: Synthetic peptide
NYGIN

SEQ ID NO: 5 - protein, 7 aa, Artificial Sequence
  Description of Artificial Sequence: Synthetic peptide
GYTFTNY

SEQ ID NO: 6 - protein, 6 aa, Artificial Sequence
  Description of Artificial Sequence: Synthetic peptide
YIGNDY

SEQ ID NO: 7 - protein, 8 aa, Artificial Sequence
  Description of Artificial Sequence: Synthetic peptide
GYTFTNYG

SEQ ID NO: 8 - protein, 5 aa, Artificial Sequence
  Description of Artificial Sequence: Synthetic peptide
IYIGN

SEQ ID NO: 9 - protein, 16 aa, Artificial Sequence
  Description of Artificial Sequence: Synthetic peptide
ARLYYGSSLYSYAMDY

SEQ ID NO: 10 - protein, 123 aa, Artificial Sequence
  Description of Artificial Sequence: Synthetic polypeptide
QVQLVQSGAEVKKPGASVKVSCKASGYTFTNYGINWVRQAPGQRLEWMGYIYIGNDYTEYNERFKGRVTITSDTSASTAYMELSSLRSEDTAVYYCARLYYGSSLYSYAMDYWGQGTTVTVSS

SEQ ID NO: 11 - DNA, 369 bp, Artificial Sequence
  Description of Artificial Sequence: Synthetic polynucleotide
caagttcagttggttcagtctggcgccgaagtgaagaaacctggcgcctctgtgaaggtgtcctgcaaggcttctggctacacctttaccaactacggcatcaactgggtccgacaggctcctggccagagattggagtggatgggctacatctacatcggcaacgactacaccgagtacaacgagcggttcaagggcagagtgaccatcacctctgacacctctgcctccaccgcctacatggaactgtccagcctgagatctgaggacaccgccgtgtactactgcgccaggctgtactatggctcctccctgtacagctatgccatggactactggggacagggcacaaccgtgacagtgagctcc

SEQ ID NO: 12 - protein, 453 aa, Artificial Sequence
  Description of Artificial Sequence: Synthetic polypeptide
QVQLVQSGAEVKKPGASVKVSCKASGYTFTNYGINWVRQAPGQRLEWMGYIYIGNDYTEYNERFKGRVTITSDTSASTAYMELSSLRSEDTAVYYCARLYYGSSLYSYAMDYWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 13 - DNA, 1359 bp, Artificial Sequence
  Description of Artificial Sequence: Synthetic polynucleotide
caagttcagttggttcagtctggcgccgaagtgaagaaacctggcgcctctgtgaaggtgtcctgcaaggcttctggctacacctttaccaactacggcatcaactgggtccgacaggctcctggccagagattggagtggatgggctacatctacatcggcaacgactacaccgagtacaacgagcggttcaagggcagagtgaccatcacctctgacacctctgcctccaccgcctacatggaactgtccagcctgagatctgaggacaccgccgtgtactactgcgccaggctgtactatggctcctccctgtacagctatgccatggactactggggacagggcacaaccgtgacagtgagctccgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 14 - protein, 16 aa, Artificial Sequence
  Description of Artificial Sequence: Synthetic peptide
RSSKSLLHSSGNTYLY

SEQ ID NO: 15 - protein, 7 aa, Artificial Sequence
  Description of Artificial Sequence: Synthetic peptide
RMSNLAS

SEQ ID NO: 16 - protein, 9 aa, Artificial Sequence
  Description of Artificial Sequence: Synthetic peptide
MQHLEYPYT

SEQ ID NO: 17 - protein, 12 aa, Artificial Sequence
  Description of Artificial Sequence: Synthetic peptide
SKSLLHSSGNTY

SEQ ID NO: 18 - protein, 3 aa, Artificial Sequence
  Description of Artificial Sequence: Synthetic peptide
RMS

SEQ ID NO: 19 - protein, 6 aa, Artificial Sequence
  Description of Artificial Sequence: Synthetic peptide
HLEYPY

SEQ ID NO: 20 - protein, 11 aa, Artificial Sequence
  Description of Artificial Sequence: Synthetic peptide
KSLLHSSGNTY

SEQ ID NO: 21 - protein, 112 aa, Artificial Sequence
  Description of Artificial Sequence: Synthetic polypeptide
DIVMTQSPLSLPVTPGEPASISCRSSKSLLHSSGNTYLYWFLQKPGQSPQLLISRMSNLASGVPDRFSGSGSGTDFTLKISRVEAEDVGVYYCMQHLEYPYTFGGGTKVEIK

SEQ ID NO: 22 - DNA, 336 bp, Artificial Sequence
  Description of Artificial Sequence: Synthetic polynucleotide
gacattgtgatgacccagtctccactgagcctgcctgtgacacctggcgagcctgcttccatctcctgccggtcctctaagtccctgctgcactcttccggcaatacctacctgtactggttcctgcagaagcccggccagtctcctcagctgctgatctccagaatgtccaacctggcctctggcgtgcccgacagattttctggctctggatctggcaccgacttcaccctgaagatctctagagtggaagccgaggacgtgggcgtgtactactgtatgcagcacctggaatacccctacaccttcggcggaggcaccaaggtggaaatcaag

SEQ ID NO: 23 - protein, 219 aa, Artificial Sequence
  Description of Artificial Sequence: Synthetic polypeptide
DIVMTQSPLSLPVTPGEPASISCRSSKSLLHSSGNTYLYWFLQKPGQSPQLLISRMSNLASGVPDRFSGSGSGTDFTLKISRVEAEDVGVYYCMQHLEYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 657 bp, Artificial Sequence
  Description of Artificial Sequence: Synthetic polynucleotide
gacattgtgatgacccagtctccactgagcctgcctgtgacacctggcgagcctgcttccatctcctgccggtcctctaagtccctgctgcactcttccggcaatacctacctgtactggttcctgcagaagcccggccagtctcctcagctgctgatctccagaatgtccaacctggcctctggcgtgcccgacagattttctggctctggatctggcaccgacttcaccctgaagatctctagagtggaagccgaggacgtgggcgtgtactactgtatgcagcacctggaatacccctacaccttcggcggaggcaccaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 25 - protein, 12 aa, Artificial Sequence
  Description of Artificial Sequence: Synthetic peptide
GFSLSTGGMSVS

SEQ ID NO: 26 - protein, 16 aa, Artificial Sequence
  Description of Artificial Sequence: Synthetic peptide
LIDWDDDKYYSTSLKT

SEQ ID NO: 27 - protein, 9 aa, Artificial Sequence
  Description of Artificial Sequence: Synthetic peptide
AHSGSYFDF

SEQ ID NO: 28 - protein, 7 aa, Artificial Sequence
  Description of Artificial Sequence: Synthetic peptide
TGGMSVS

SEQ ID NO: 29 - protein, 9 aa, Artificial Sequence
  Description of Artificial Sequence: Synthetic peptide
GFSLSTGGM

SEQ ID NO: 30 - protein, 5 aa, Artificial Sequence
  Description of Artificial Sequence: Synthetic peptide
DWDDD

SEQ ID NO: 31 - protein, 10 aa, Artificial Sequence
  Description of Artificial Sequence: Synthetic peptide
GFSLSTGGMS

SEQ ID NO: 32 - protein, 7 aa, Artificial Sequence
  Description of Artificial Sequence: Synthetic peptide
IDWDDDK

SEQ ID NO: 33 - protein, 11 aa, Artificial Sequence
  Description of Artificial Sequence: Synthetic peptide
ARAHSGSYFDF

SEQ ID NO: 34 - protein, 119 aa, Artificial Sequence
  Description of Artificial Sequence: Synthetic polypeptide
QVTLRESGPALVKPTQTLTLTCTFSGFSLSTGGMSVSWIRQPPGKALEWLALIDWDDDKYYSTSLKTRLTISKDTSKNQLVLTMTNMDPVDTATYYCARAHSGSYFDFWGQGTLVTVSS

SEQ ID NO: 35 - DNA, 357 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactggtggaatgagtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctca

SEQ ID NO: 36 - protein, 449 aa, Artificial Sequence
  Description of Artificial Sequence: Synthetic polypeptide
QVTLRESGPALVKPTQTLTLTCTFSGFSLSTGGMSVSWIRQPPGKALEWLALIDWDDDKYYSTSLKTRLTISKDTSKNQLVLTMTNMDPVDTATYYCARAHSGSYFDFWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 37 - DNA, 1347 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactggtggaatgagtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 38 - protein, 11 aa, Artificial Sequence
  Description of Artificial Sequence: Synthetic peptide
RASQRISNWLA

SEQ ID NO: 39 - protein, 7 aa, Artificial Sequence
  Description of Artificial Sequence: Synthetic peptide
KASSLES

SEQ ID NO: 40 - protein, 8 aa, Artificial Sequence
  Description of Artificial Sequence: Synthetic peptide
QQFSSYWT

SEQ ID NO: 41 - protein, 7 aa, Artificial Sequence
  Description of Artificial Sequence: Synthetic peptide
SQRISNW

SEQ ID NO: 42 - protein, 3 aa, Artificial Sequence
  Description of Artificial Sequence: Synthetic peptide
KAS

SEQ ID NO: 43 - protein, 5 aa, Artificial Sequence
  Description of Artificial Sequence: Synthetic peptide
FSSYW

SEQ ID NO: 44 - protein, 6 aa, Artificial Sequence
  Description of Artificial Sequence: Synthetic peptide
QRISNW

SEQ ID NO: 45 - protein, 106 aa, Artificial Sequence
  Description of Artificial Sequence: Synthetic polypeptide
DIQMTQSPSTLSASVGDRVTITCRASQRISNWLAWYQQKPGKAPKLLIYKASSLESGVPSRFSGSGSGTEFTLTISSLQPDDFATYYCQQFSSYWTFGQGTKVEIK

SEQ ID NO: 46 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttagtagttattggacgttcggccaagggaccaaggtggaaatcaaa

SEQ ID NO: 47 - protein, 213 aa, Artificial Sequence
  Description of Artificial Sequence: Synthetic polypeptide
DIQMTQSPSTLSASVGDRVTITCRASQRISNWLAWYQQKPGKAPKLLIYKASSLESGVPSRFSGSGSGTEFTLTISSLQPDDFATYYCQQFSSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttagtagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 49 - protein, 12 aa, Artificial Sequence
  Description of Artificial Sequence: Synthetic peptide
GFSLSTSGISVS

SEQ ID NO: 50 - protein, 12 aa, Artificial Sequence
  Description of Artificial Sequence: Synthetic peptide
TPSGSYGRYFDL

SEQ ID NO: 51 - protein, 7 aa, Artificial Sequence
  Description of Artificial Sequence: Synthetic peptide
TSGISVS

SEQ ID NO: 52 - protein, 9 aa, Artificial Sequence
  Description of Artificial Sequence: Synthetic peptide
GFSLSTSGI

SEQ ID NO: 53 - protein, 10 aa, Artificial Sequence
  Description of Artificial Sequence: Synthetic peptide
GFSLSTSGIS

SEQ ID NO: 54 - protein, 14 aa, Artificial Sequence
  Description of Artificial Sequence: Synthetic peptide
ARTPSGSYGRYFDL

SEQ ID NO: 55 - protein, 122 aa, Artificial Sequence
  Description of Artificial Sequence: Synthetic polypeptide
QVTLRESGPALVKPTQTLTLTCTFSGFSLSTSGISVSWIRQPPGKALEWLALIDWDDDKYYSTSLKTRLTISKDTSKNQVVLTMTNMDPVDTATYYCARTPSGSYGRYFDLWGRGTLVTVSS

SEQ ID NO: 56 - DNA, 366 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcacaagtggaatatctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggacccctagtgggagctatgggcgatacttcgatctctggggccgtggcaccctggtcactgtctcctca

SEQ ID NO: 57 - protein, 452 aa, Artificial Sequence
  Description of Artificial Sequence: Synthetic polypeptide
QVTLRESGPALVKPTQTLTLTCTFSGFSLSTSGISVSWIRQPPGKALEWLALIDWDDDKYYSTSLKTRLTISKDTSKNQVVLTMTNMDPVDTATYYCARTPSGSYGRYFDLWGRGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 58 - DNA, 1356 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcacaagtggaatatctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggacccctagtgggagctatgggcgatacttcgatctctggggccgtggcaccctggtcactgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 59 - protein, 11 aa, Artificial Sequence
  Description of Artificial Sequence: Synthetic peptide
RASQSISNWLA

SEQ ID NO: 60 - protein, 8 aa, Artificial Sequence
  Description of Artificial Sequence: Synthetic peptide
QQYNAYWT

SEQ ID NO: 61 - protein, 7 aa, Artificial Sequence
  Description of Artificial Sequence: Synthetic peptide
SQSISNW

SEQ ID NO: 62 - protein, 5 aa, Artificial Sequence
  Description of Artificial Sequence: Synthetic peptide
YNAYW

SEQ ID NO: 63 - protein, 6 aa, Artificial Sequence
  Description of Artificial Sequence: Synthetic peptide
QSISNW

SEQ ID NO: 64 - protein, 106 aa, Artificial Sequence
  Description of Artificial Sequence: Synthetic polypeptide
DIQLTQSPSTLSASVGDRVTITCRASQSISNWLAWYQQKPGKAPKLLIYKASSLESGVPSRFTGSGSGTEFTLTISSLQPDDFATYYCQQYNAYWTFGQGTKVEIK

SEQ ID NO: 65 - DNA, 318 bp, Artificial Sequence
  Description of Artificial Sequence: Synthetic polynucleotide
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatgcctattggacgttcggccaagggaccaaggtggaaatcaaa

SEQ ID NO: 66 - protein, 213 aa, Artificial Sequence
  Description of Artificial Sequence: Synthetic polypeptide
DIQLTQSPSTLSASVGDRVTITCRASQSISNWLAWYQQKPGKAPKLLIYKASSLESGVPSRFTGSGSGTEFTLTISSLQPDDFATYYCQQYNAYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 639 bp, Artificial Sequence
  Description of Artificial Sequence: Synthetic polynucleotide
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatgcctattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 68 - protein, 8 aa, Artificial Sequence
  Description of Artificial Sequence: Synthetic peptide
QQFQSYWT

SEQ ID NO: 69 - protein, 5 aa, Artificial Sequence
  Description of Artificial Sequence: Synthetic peptide
FQSYW

SEQ ID NO: 70 - protein, 106 aa, Artificial Sequence
  Description of Artificial Sequence: Synthetic polypeptide
DIQMTQSPSTLSASVGDRVTITCRASQRISNWLAWYQQKPGKAPKLLIYKASSLESGVPSRFSGSGSGTEFTLTISSLQPDDFATYYCQQFQSYWTFGQGTKVEIK

SEQ ID NO: 71 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttcagagttattggacgttcggccaagggaccaaggtggaaatcaaa

SEQ ID NO: 72 - protein, 213 aa, Artificial Sequence
  Description of Artificial Sequence: Synthetic polypeptide
DIQMTQSPSTLSASVGDRVTITCRASQRISNWLAWYQQKPGKAPKLLIYKASSLESGVPSRFSGSGSGTEFTLTISSLQPDDFATYYCQQFQSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttcagagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 74 - protein, 12 aa, Artificial Sequence
  Description of Artificial Sequence: Synthetic peptide
GFSLSTSGVSVS

SEQ ID NO: 75 - protein, 7 aa, Artificial Sequence
  Description of Artificial Sequence: Synthetic peptide
TSGVSVS

SEQ ID NO: 76 - protein, 9 aa, Artificial Sequence
  Description of Artificial Sequence: Synthetic peptide
GFSLSTSGV

SEQ ID NO: 77 - protein, 10 aa, Artificial Sequence
  Description of Artificial Sequence: Synthetic peptide
GFSLSTSGVS

SEQ ID NO: 78 - protein, 122 aa, Artificial Sequence
  Description of Artificial Sequence: Synthetic polypeptide
QVTLRESGPALVKPTQTLTLTCTFSGFSLSTSGVSVSWIRQPPGKALEWLALIDWDDDKYYSTSLKTRLTISKDTSKNQVVLTMTNMDPVDTATYYCARTPSGSYGRYFDLWGRGTLVTVSS

SEQ ID NO: 79 - DNA, 366 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactagtggagtgtctgtgagttggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtactattgtgcacggacccctagtgggagctacgggcgatacttcgatctctggggccgtggcaccctggtcactgtctcctca

SEQ ID NO: 80 - protein, 452 aa, Artificial Sequence
  Description of Artificial Sequence: Synthetic polypeptide
QVTLRESGPALVKPTQTLTLTCTFSGFSLSTSGVSVSWIRQPPGKALEWLALIDWDDDKYYSTSLKTRLTISKDTSKNQVVLTMTNMDPVDTATYYCARTPSGSYGRYFDLWGRGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 81 - DNA, 1356 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactagtggagtgtctgtgagttggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtactattgtgcacggacccctagtgggagctacgggcgatacttcgatctctggggccgtggcaccctggtcactgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 82 - protein, 8 aa, Artificial Sequence
  Description of Artificial Sequence: Synthetic peptide
QQYQSYWT

SEQ ID NO: 83 - protein, 5 aa, Artificial Sequence
  Description of Artificial Sequence: Synthetic peptide
YQSYW

SEQ ID NO: 84 - protein, 106 aa, Artificial Sequence
  Description of Artificial Sequence: Synthetic polypeptide
DIQMTQSPSTLSASVGDRVTITCRASQSISNWLAWYQQKPGKAPKLLIYKASSLESGVPSRFSGSGSGTEFTLTISSLQPDDFATYYCQQYQSYWTFGQGTKVEIK

SEQ ID NO: 85 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaa

SEQ ID NO: 86 - protein, 213 aa, Artificial Sequence
  Description of Artificial Sequence: Synthetic polypeptide
DIQMTQSPSTLSASVGDRVTITCRASQSISNWLAWYQQKPGKAPKLLIYKASSLESGVPSRFSGSGSGTEFTLTISSLQPDDFATYYCQQYQSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 88 - protein, 12 aa, Artificial Sequence
  Description of Artificial Sequence: Synthetic peptide
GFSLSTSGVSVT

SEQ ID NO: 89 - protein, 7 aa, Artificial Sequence
  Description of Artificial Sequence: Synthetic peptide
TSGVSVT

SEQ ID NO: 90 - protein, 122 aa, Artificial Sequence
  Description of Artificial Sequence: Synthetic polypeptide
QVTLRESGPALVKPTQTLTLTCTFSGFSLSTSGVSVTWIRQPPGKALEWLALIDWDDDKYYSTSLKTRLAISKDTSKNQVVLTMTNMDPVDTATYYCARTPSGSYGRYFDLWGRGTLVTVSS

SEQ ID NO: 91 - DNA, 366 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactagtggagtgtctgtgacctggatccgtcagcccccagggaaggccctggagtggcttgcactcattgattgggatgatgataaatactacagcacatctctgaagaccaggctcgccatctccaaggacacctccaaaaaccaggtggtccttacaatgaccaacatggaccctgtggacacagccacgtattattgtgcacggacccctagtgggagctacgggcgatacttcgatctctggggccgtggcaccctggtcactgtctcctca

SEQ ID NO: 92 - protein, 452 aa, Artificial Sequence
  Description of Artificial Sequence: Synthetic polypeptide
QVTLRESGPALVKPTQTLTLTCTFSGFSLSTSGVSVTWIRQPPGKALEWLALIDWDDDKYYSTSLKTRLAISKDTSKNQVVLTMTNMDPVDTATYYCARTPSGSYGRYFDLWGRGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 93 - DNA, 1356 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactagtggagtgtctgtgacctggatccgtcagcccccagggaaggccctggagtggcttgcactcattgattgggatgatgataaatactacagcacatctctgaagaccaggctcgccatctccaaggacacctccaaaaaccaggtggtccttacaatgaccaacatggaccctgtggacacagccacgtattattgtgcacggacccctagtgggagctacgggcgatacttcgatctctggggccgtggcaccctggtcactgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 94 - protein, 11 aa, Artificial Sequence
  Description of Artificial Sequence: Synthetic peptide
RASQSISTWLA

SEQ ID NO: 95 - protein, 7 aa, Artificial Sequence
  Description of Artificial Sequence: Synthetic peptide
EASSLES

SEQ ID NO: 96 - protein, 7 aa, Artificial Sequence
  Description of Artificial Sequence: Synthetic peptide
SQSISTW

SEQ ID NO: 97 - protein, 3 aa, Artificial Sequence
  Description of Artificial Sequence: Synthetic peptide
EAS

SEQ ID NO: 98 - protein, 6 aa, Artificial Sequence
  Description of Artificial Sequence: Synthetic peptide
QSISTW

SEQ ID NO: 99 - protein, 106 aa, Artificial Sequence
  Description of Artificial Sequence: Synthetic polypeptide
DIQMTQSPSTLSASVGDRVTITCRASQSISTWLAWYQQKPGKAPKLLIYEASSLESGVPSRFSGSGSGTEFTLTISSLQPDDFATYYCQQYQSYWTFGQGTKVEIK

SEQ ID NO: 100 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaa

SEQ ID NO: 101 - protein, 213 aa, Artificial Sequence
  Description of Artificial Sequence: Synthetic polypeptide
DIQMTQSPSTLSASVGDRVTITCRASQSISTWLAWYQQKPGKAPKLLIYEASSLESGVPSRFSGSGSGTEFTLTISSLQPDDFATYYCQQYQSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtatcagagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 103 - protein, 123 aa, Artificial Sequence
  Description of Artificial Sequence: Synthetic polypeptide
EVQLQQSGAELVRPGSSVKMSCKTSGYTFTNYGINWVKQRPGQGLEWIGYIYIGNDYTEYNERFKGKATLTSDTSSSTAHIQLNSLTSEDSAIYFCARLYYGSSLYSYAMDYWGQGTSVTVSS

SEQ ID NO: 104 - DNA, 369 bp, Artificial Sequence
  Description of Artificial Sequence: Synthetic polynucleotide
gaggttcagctgcagcagtctggagctgagttggtgaggcctgggtcctcagtgaagatgtcctgcaagacttctggatatacattcacaaactacggtataaactgggtgaagcagaggcctggacagggcctggaatggattggatatatttatattggaaatgattatactgagtacaatgagaggttcaagggcaaggccacactgacttcagacacatcctccagcacagcccacatacaactcaacagcctgacatctgaggactctgcaatctatttctgtgcaagactttactacggtagtagcctctattcttatgctatggactactggggtcaaggaacctctgtcacagtctcctct

SEQ ID NO: 105 - protein, 453 aa, Artificial Sequence
  Description of Artificial Sequence: Synthetic polypeptide
EVQLQQSGAELVRPGSSVKMSCKTSGYTFTNYGINWVKQRPGQGLEWIGYIYIGNDYTEYNERFKGKATLTSDTSSSTAHIQLNSLTSEDSAIYFCARLYYGSSLYSYAMDYWGQGTSVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 106 - DNA, 1359 bp, Artificial Sequence
  Description of Artificial Sequence: Synthetic polynucleotide
gaggttcagctgcagcagtctggagctgagttggtgaggcctgggtcctcagtgaagatgtcctgcaagacttctggatatacattcacaaactacggtataaactgggtgaagcagaggcctggacagggcctggaatggattggatatatttatattggaaatgattatactgagtacaatgagaggttcaagggcaaggccacactgacttcagacacatcctccagcacagcccacatacaactcaacagcctgacatctgaggactctgcaatctatttctgtgcaagactttactacggtagtagcctctattcttatgctatggactactggggtcaaggaacctctgtcacagtctcctctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 107 - protein, 112 aa, Artificial Sequence
  Description of Artificial Sequence: Synthetic polypeptide
DIVMTQAAPSVSVTPGESVSISCRSSKSLLHSSGNTYLYWFLQRPGQSPQLLISRMSNLASGVPDRFSGSGSGTAFTLRISRVEAEDVGVYYCMQHLEYPYTFGGGTKLELK

SEQ ID NO: 108 - DNA, 336 bp, Artificial Sequence
  Description of Artificial Sequence: Synthetic polynucleotide
gatattgtgatgactcaggctgcaccctctgtatctgtcactcctggagagtcagtatccatctcctgcaggtctagtaagagtctcctccatagtagtggcaacacttacttgtattggttcctgcagaggccaggccagtctcctcagctcctgatatctcggatgtccaaccttgcctcaggagtcccagacaggttcagtggcagtgggtcaggaactgctttcacactgagaatcagtagagtggaggctgaggatgtgggtgtttattattgtatgcaacatctagaatatccgtacacgttcggaggggggaccaagctggagctaaaa

SEQ ID NO: 109 - protein, 219 aa, Artificial Sequence
  Description of Artificial Sequence: Synthetic polypeptide
DIVMTQAAPSVSVTPGESVSISCRSSKSLLHSSGNTYLYWFLQRPGQSPQLLISRMSNLASGVPDRFSGSGSGTAFTLRISRVEAEDVGVYYCMQHLEYPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - DNA, 657 bp, Artificial Sequence
  Description of Artificial Sequence: Synthetic polynucleotide
gatattgtgatgactcaggctgcaccctctgtatctgtcactcctggagagtcagtatccatctcctgcaggtctagtaagagtctcctccatagtagtggcaacacttacttgtattggttcctgcagaggccaggccagtctcctcagctcctgatatctcggatgtccaaccttgcctcaggagtcccagacaggttcagtggcagtgggtcaggaactgctttcacactgagaatcagtagagtggaggctgaggatgtgggtgtttattattgtatgcaacatctagaatatccgtacacgttcggaggggggaccaagctggagctaaaacgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 111 - protein, 12 aa, Artificial Sequence
  Description of Artificial Sequence: Synthetic peptide
GFSLSTGGMCVS

SEQ ID NO: 112 - protein, 7 aa, Artificial Sequence
  Description of Artificial Sequence: Synthetic peptide
TGGMCVS

SEQ ID NO: 113 - protein, 10 aa, Artificial Sequence
  Description of Artificial Sequence: Synthetic peptide
GFSLSTGGMC

SEQ ID NO: 114 - protein, 119 aa, Artificial Sequence
  Description of Artificial Sequence: Synthetic polypeptide
QVTLRESGPALVKPTQTLTLTCTFSGFSLSTGGMCVSWIRQPPGKALEWLALIDWDDDKYYSTSLKTRLTISKDTSKNQLVLTMTNMDPVDTATYYCARAHSGSYFDFWGQGTLVTVSS

SEQ ID NO: 115 - DNA, 357 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactggtggaatgtgtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctca

SEQ ID NO: 116 - protein, 449 aa, Artificial Sequence
  Description of Artificial Sequence: Synthetic polypeptide
QVTLRESGPALVKPTQTLTLTCTFSGFSLSTGGMCVSWIRQPPGKALEWLALIDWDDDKYYSTSLKTRLTISKDTSKNQLVLTMTNMDPVDTATYYCARAHSGSYFDFWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 117 - DNA, 1347 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcactcagcactggtggaatgtgtgtgagctggatccgtcagcccccagggaaggccctggagtggcttgcactcattgattgggatgatgataaatactacagcacatctctgaagaccaggctcaccatctccaaggacacctccaaaaaccagctggtccttacaatgaccaacatggaccctgtggacacagccacgtattattgtgcacgggctcatagtgggagctactttgacttctggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 118 - protein, 8 aa, Artificial Sequence
  Description of Artificial Sequence: Synthetic peptide
QQFNSYWT

SEQ ID NO: 119 - protein, 5 aa, Artificial Sequence
  Description of Artificial Sequence: Synthetic peptide
FNSYW

SEQ ID NO: 120 - protein, 106 aa, Artificial Sequence
  Description of Artificial Sequence: Synthetic polypeptide
DIQMTQSPSTLSASVGDRVTITCRASQRISNWLAWYQQKPGKAPKLLIYKASSLESGVPSRFSGSGSGTEFTLTISSLQPDDFATYYCQQFNSYWTFGQGTKVEIK

SEQ ID NO: 121 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaa

SEQ ID NO: 122 - protein, 213 aa, Artificial Sequence
  Description of Artificial Sequence: Synthetic polypeptide
DIQMTQSPSTLSASVGDRVTITCRASQRISNWLAWYQQKPGKAPKLLIYKASSLESGVPSRFSGSGSGTEFTLTISSLQPDDFATYYCQQFNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagaattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 124 - protein, 8 aa, Artificial Sequence
  Description of Artificial Sequence: Synthetic peptide
QQYNSYWT

SEQ ID NO: 125 - protein, 5 aa, Artificial Sequence
  Description of Artificial Sequence: Synthetic peptide
YNSYW

SEQ ID NO: 126 - protein, 106 aa, Artificial Sequence
  Description of Artificial Sequence: Synthetic polypeptide
DIQLTQSPSTLSASVGDRVTITCRASQSISNWLAWYQQKPGKAPKLLIYKASSLESGVPSRFTGSGSGTEFTLTISSLQPDDFATYYCQQYNSYWTFGQGTKVEIK

SEQ ID NO: 127 - DNA, 318 bp, Artificial Sequence
  Description of Artificial Sequence: Synthetic polynucleotide
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatagttattggacgttcggccaagggaccaaggtggaaatcaaa

SEQ ID NO: 128 - protein, 213 aa, Artificial Sequence
  Description of Artificial Sequence: Synthetic polypeptide
DIQLTQSPSTLSASVGDRVTITCRASQSISNWLAWYQQKPGKAPKLLIYKASSLESGVPSRFTGSGSGTEFTLTISSLQPDDFATYYCQQYNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - DNA, 639 bp, Artificial Sequence
  Description of Artificial Sequence: Synthetic polynucleotide
gacatccagttgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccgtcaaggttcaccggcagtggatctgggacagaattcactctcaccatcagcagcctgcagcctgatgattttgcaacttattactgccaacagtataatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 130 - protein, 106 aa, Artificial Sequence
  Description of Artificial Sequence: Synthetic polypeptide
DIQMTQSPSTLSASVGDRVTITCRASQSISNWLAWYQQKPGKAPKLLIYKASSLESGVPSRFSGSGSGTEFTLTISSLQPDDFATYYCQQYNSYWTFGQGTKVEIK

SEQ ID NO: 131 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaa

SEQ ID NO: 132 - protein, 213 aa, Artificial Sequence
  Description of Artificial Sequence: Synthetic polypeptide
DIQMTQSPSTLSASVGDRVTITCRASQSISNWLAWYQQKPGKAPKLLIYKASSLESGVPSRFSGSGSGTEFTLTISSLQPDDFATYYCQQYNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 134 - protein, 106 aa, Artificial Sequence
  Description of Artificial Sequence: Synthetic polypeptide
DIQMTQSPSTLSASVGDRVTITCRASQSISTWLAWYQQKPGKAPKLLIYEASSLESGVPSRFSGSGSGTEFTLTISSLQPDDFATYYCQQYNSYWTFGQGTKVEIK

SEQ ID NO: 135 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaa

SEQ ID NO: 136 - protein, 213 aa, Artificial Sequence
  Description of Artificial Sequence: Synthetic polypeptide
DIQMTQSPSTLSASVGDRVTITCRASQSISTWLAWYQQKPGKAPKLLIYEASSLESGVPSRFSGSGSGTEFTLTISSLQPDDFATYYCQQYNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtacctggttggcctggtatcagcagaaaccagggaaagcccctaagctcctgatctatgaggcgtctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 138 - protein, 12 aa, Artificial Sequence
  Description of Artificial Sequence: Synthetic peptide
GFSPSTSGMSVS

SEQ ID NO: 139 - protein, 16 aa, Artificial Sequence
  Description of Artificial Sequence: Synthetic peptide
LIDWDDDKYFSTSLKT

SEQ ID NO: 140 - protein, 9 aa, Artificial Sequence
  Description of Artificial Sequence: Synthetic peptide
AHSGSYFDY

SEQ ID NO: 141 - protein, 7 aa, Artificial Sequence
  Description of Artificial Sequence: Synthetic peptide
TSGMSVS

SEQ ID NO: 142 - protein, 9 aa, Artificial Sequence
  Description of Artificial Sequence: Synthetic peptide
GFSPSTSGM

SEQ ID NO: 143 - protein, 10 aa, Artificial Sequence
  Description of Artificial Sequence: Synthetic peptide
GFSPSTSGMS

SEQ ID NO: 144 - protein, 11 aa, Artificial Sequence
  Description of Artificial Sequence: Synthetic peptide
ARAHSGSYFDY

SEQ ID NO: 145 - protein, 119 aa, Artificial Sequence
  Description of Artificial Sequence: Synthetic polypeptide
QVTLRESGPALVKPTQTLTLTCTFSGFSPSTSGMSVSWIRQPPGKALEWLALIDWDDDKYFSTSLKTRLTISKDTSKNQVVLTMTNMDPVDTATYYCARAHSGSYFDYWGQGTLVTVSS

SEQ ID NO: 146 - DNA, 357 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcacccagcactagtggaatgtctgtgagctggatccgtcagcccccagggaaggccctggagtggcttgcacttattgattgggatgatgataaatactttagcacatctctgaagaccaggctcaccatctccaaggacacctccaaaaaccaggtggtccttacaatgaccaacatggaccctgtagacacagccacgtattattgtgcacgggcccatagtgggagctactttgactactggggccagggaaccctggtcaccgtctcctca

SEQ ID NO: 147 - protein, 449 aa, Artificial Sequence
  Description of Artificial Sequence: Synthetic polypeptide
QVTLRESGPALVKPTQTLTLTCTFSGFSPSTSGMSVSWIRQPPGKALEWLALIDWDDDKYFSTSLKTRLTISKDTSKNQVVLTMTNMDPVDTATYYCARAHSGSYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 148 - DNA, 1347 bp, Artificial Sequence
  Description of Artificial Sequence: Synthetic polynucleotide
caggtcaccttgagggagtctggtcctgcgctggtgaaacccacacagaccctcacactgacctgcaccttctctgggttctcacccagcactagtggaatgtctgtgagctggatccgtcagcccccagggaaggccctggagtggcttgcacttattgattgggatgatgataaatactttagcacatctctgaagaccaggctcaccatctccaaggacacctccaaaaaccaggtggtccttacaatgaccaacatggaccctgtagacacagccacgtattattgtgcacgggcccatagtgggagctactttgactactggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 149 - protein, 106 aa, Artificial Sequence
  Description of Artificial Sequence: Synthetic polypeptide
DIQMTQSPSTLSASVGDRVTITCRASQSISNWLAWYQQKPGKAPKLLIYKASSLESGVPSRFSGSGSGTEFTLTISSLQPDDFATYYCQQFNSYWTFGQGTKVEIK

SEQ ID NO: 150 - DNA, 318 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaa

SEQ ID NO: 151 - protein, 213 aa, Artificial Sequence
  Description of Artificial Sequence: Synthetic polypeptide
DIQMTQSPSTLSASVGDRVTITCRASQSISNWLAWYQQKPGKAPKLLIYKASSLESGVPSRFSGSGSGTEFTLTISSLQPDDFATYYCQQFNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - DNA, 639 bp, Artificial Sequence
  Description of Artificial Sequence: Synthetic polynucleotide
gacatccagatgacccagtctccttccaccctgtctgcatctgtaggagacagagtcaccatcacttgccgggccagtcagagtattagtaactggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtttaatagttattggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 153 - protein, 10 aa, Artificial Sequence
  Description of Artificial Sequence: Synthetic peptide
GGSISSYYWN

SEQ ID NO: 154 - protein, 16 aa, Artificial Sequence
  Description of Artificial Sequence: Synthetic peptide
RIYTSGSTNYNPSLKS

SEQ ID NO: 155 - protein, 13 aa, Artificial Sequence
  Description of Artificial Sequence: Synthetic peptide
DSGGFYYYYGMDV

SEQ ID NO: 156 - protein, 5 aa, Artificial Sequence
  Description of Artificial Sequence: Synthetic peptide
SYYWN

SEQ ID NO: 157 - protein, 7 aa, Artificial Sequence
  Description of Artificial Sequence: Synthetic peptide
GGSISSY

SEQ ID NO: 158 - protein, 5 aa, Artificial Sequence
  Description of Artificial Sequence: Synthetic peptide
YTSGS

SEQ ID NO: 159 - protein, 8 aa, Artificial Sequence
  Description of Artificial Sequence: Synthetic peptide
GGSISSYY

SEQ ID NO: 160 - protein, 7 aa, Artificial Sequence
  Description of Artificial Sequence: Synthetic peptide
IYTSGST

SEQ ID NO: 161 - protein, 15 aa, Artificial Sequence
  Description of Artificial Sequence: Synthetic peptide
ARDSGGFYYYYGMDV

SEQ ID NO: 162 - protein, 121 aa, Artificial Sequence
  Description of Artificial Sequence: Synthetic polypeptide
QVQLQESGPGLVKPSETLSLTCAVSGGSISSYYWNLIRQPAGKGLEWIGRIYTSGSTNYNPSLKSRVTMSVDTSKNQFSLKLSSVTAADTAVYYCARDSGGFYYYYGMDVWGQGTTVTVSS

SEQ ID NO: 163 - DNA, 363 bp, Artificial Sequence
  Description of Artificial Sequence: Synthetic polynucleotide
caggtgcagctgcaggagtcgggcccaggactggtgaagccttcggagaccctgtccctcacctgcgctgtctctggtggctccatcagtagttactactggaacttaatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagactccggggggttctactactactacggtatggacgtctggggccaagggaccacggtcaccgtctcctca

SEQ ID NO: 164 - protein, 451 aa, Artificial Sequence
  Description of Artificial Sequence: Synthetic polypeptide
QVQLQESGPGLVKPSETLSLTCAVSGGSISSYYWNLIRQPAGKGLEWIGRIYTSGSTNYNPSLKSRVTMSVDTSKNQFSLKLSSVTAADTAVYYCARDSGGFYYYYGMDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 165 - DNA, 1353 bp, Artificial Sequence
  Description of Artificial Sequence: Synthetic polynucleotide
caggtgcagctgcaggagtcgggcccaggactggtgaagccttcggagaccctgtccctcacctgcgctgtctctggtggctccatcagtagttactactggaacttaatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagactccggggggttctactactactacggtatggacgtctggggccaagggaccacggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 166 - protein, 11 aa, Artificial Sequence
  Description of Artificial Sequence: Synthetic peptide
RASQGISSYLA

SEQ ID NO: 167 - protein, 7 aa, Artificial Sequence
  Description of Artificial Sequence: Synthetic peptide
AASTLQG

SEQ ID NO: 168 - protein, 9 aa, Artificial Sequence
  Description of Artificial Sequence: Synthetic peptide
QQLNSYPWT

SEQ ID NO: 169 - protein, 7 aa, Artificial Sequence
  Description of Artificial Sequence: Synthetic peptide
SQGISSY

SEQ ID NO: 170 - protein, 3 aa, Artificial Sequence
  Description of Artificial Sequence: Synthetic peptide
AAS

SEQ ID NO: 171 - protein, 6 aa, Artificial Sequence
  Description of Artificial Sequence: Synthetic peptide
LNSYPW

SEQ ID NO: 172 - protein, 6 aa, Artificial Sequence
  Description of Artificial Sequence: Synthetic peptide
QGISSY

SEQ ID NO: 173 - protein, 10 aa, Artificial Sequence
  Description of Artificial Sequence: Synthetic peptide
AASTLQGGVP

SEQ ID NO: 174 - protein, 107 aa, Artificial Sequence
  Description of Artificial Sequence: Synthetic polypeptide
DIQLTQSPSFLSASVGDRVTITCRASQGISSYLAWYQQKPGKAPKLLIYAASTLQGGVPSRFSGSGSGTEFTLTISSLQPEDFATYHCQQLNSYPWTFGQGTKVEIK

SEQ ID NO: 175 - DNA, 321 bp, Artificial Sequence
  Description of Artificial Sequence: Synthetic polynucleotide
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccaccttgcaaggtggggtcccatcaaggttcagcggcagtggatctgggacagaattcactctcacaatcagcagcctgcagcctgaagattttgcaacttatcactgtcaacagcttaatagttacccgtggacgttcggccaagggaccaaggtggaaatcaaa

SEQ ID NO: 176 - protein, 214 aa, Artificial Sequence
  Description of Artificial Sequence: Synthetic polypeptide
DIQLTQSPSFLSASVGDRVTITCRASQGISSYLAWYQQKPGKAPKLLIYAASTLQGGVPSRFSGSGSGTEFTLTISSLQPEDFATYHCQQLNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - DNA, 642 bp, Artificial Sequence
  Description of Artificial Sequence: Synthetic polynucleotide
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccaccttgcaaggtggggtcccatcaaggttcagcggcagtggatctgggacagaattcactctcacaatcagcagcctgcagcctgaagattttgcaacttatcactgtcaacagcttaatagttacccgtggacgttcggccaagggaccaaggtggaaatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 178 - protein, 10 aa, Artificial Sequence
  Description of Artificial Sequence: Synthetic peptide
GFTFNNYWMN

SEQ ID NO: 179 - protein, 17 aa, Artificial Sequence
  Description of Artificial Sequence: Synthetic peptide
NIRQDGSEKYYVDSVKG

SEQ ID NO: 180 - protein, 17 aa, Artificial Sequence
  Description of Artificial Sequence: Synthetic peptide
ERAYCSTTSCPDYSNDY

SEQ ID NO: 181 - protein, 5 aa, Artificial Sequence
  Description of Artificial Sequence: Synthetic peptide
NYWMN

SEQ ID NO: 182 - protein, 7 aa, Artificial Sequence
  Description of Artificial Sequence: Synthetic peptide
GFTFNNY

SEQ ID NO: 183 - protein, 6 aa, Artificial Sequence
  Description of Artificial Sequence: Synthetic peptide
RQDGSE

SEQ ID NO: 184 - protein, 8 aa, Artificial Sequence
  Description of Artificial Sequence: Synthetic peptide
GFTFNNYW

SEQ ID NO: 185 - protein, 8 aa, Artificial Sequence
  Description of Artificial Sequence: Synthetic peptide
IRQDGSEK

SEQ ID NO: 186 - protein, 19 aa, Artificial Sequence
  Description of Artificial Sequence: Synthetic peptide
ARERAYCSTTSCPDYSNDY

SEQ ID NO: 187 - protein, 126 aa, Artificial Sequence
  Description of Artificial Sequence: Synthetic polypeptide
EVQLVESGGGLVQPGGSLRLSCAASGFTFNNYWMNWVRQAPGKGLEWVANIRQDGSEKYYVDSVKGRFTISRDNAKNSLFLQMNSLRAEDTAVYYCARERAYCSTTSCPDYSNDYWGQGTLVTVSS

SEQ ID NO: 188 - DNA, 378 bp, Artificial Sequence
  Description of Artificial Sequence: Synthetic polynucleotide
gaggtgcagctggtggagtctgggggaggcttggtccagcctggggggtccctgaggctctcctgtgcagcctctggattcacctttaataactattggatgaactgggtccgccaggctccagggaaggggctggagtgggtggccaacataagacaagatggaagtgaaaaatactatgtggactctgtgaagggccgattcaccatctccagagacaacgccaagaactcactgtttctacaaatgaacagcctgagagccgaggacacggctgtatattactgtgcgagagagagggcctattgtagtactaccagctgccctgactacagtaatgactactggggccagggaaccctggtcaccgtctcctca

SEQ ID NO: 189 - protein, 456 aa, Artificial Sequence
  Description of Artificial Sequence: Synthetic polypeptide
EVQLVESGGGLVQPGGSLRLSCAASGFTFNNYWMNWVRQAPGKGLEWVANIRQDGSEKYYVDSVKGRFTISRDNAKNSLFLQMNSLRAEDTAVYYCARERAYCSTTSCPDYSNDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 190 - DNA, 1368 bp, Artificial Sequence
  Description of Artificial Sequence: Synthetic polynucleotide
gaggtgcagctggtggagtctgggggaggcttggtccagcctggggggtccctgaggctctcctgtgcagcctctggattcacctttaataactattggatgaactgggtccgccaggctccagggaaggggctggagtgggtggccaacataagacaagatggaagtgaaaaatactatgtggactctgtgaagggccgattcaccatctccagagacaacgccaagaactcactgtttctacaaatgaacagcctgagagccgaggacacggctgtatattactgtgcgagagagagggcctattgtagtactaccagctgccctgactacagtaatgactactggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 191 - protein, 11 aa, Artificial Sequence
  Description of Artificial Sequence: Synthetic peptide
RASQTINNNLA

SEQ ID NO: 192 - protein, 7 aa, Artificial Sequence
  Description of Artificial Sequence: Synthetic peptide
GASTGAT

SEQ ID NO: 193 - protein, 11 aa, Artificial Sequence
  Description of Artificial Sequence: Synthetic peptide
QQYNNWPRGLT

SEQ ID NO: 194 - protein, 7 aa, Artificial Sequence
  Description of Artificial Sequence: Synthetic peptide
SQTINNN

SEQ ID NO: 195 - protein, 3 aa, Artificial Sequence
  Description of Artificial Sequence: Synthetic peptide
GAS

SEQ ID NO: 196 - protein, 8 aa, Artificial Sequence
  Description of Artificial Sequence: Synthetic peptide
YNNWPRGL

SEQ ID NO: 197 - protein, 6 aa, Artificial Sequence
  Description of Artificial Sequence: Synthetic peptide
QTINNN

SEQ ID NO: 198 - protein, 10 aa, Artificial Sequence
  Description of Artificial Sequence: Synthetic peptide
GASTGATGIP

SEQ ID NO: 199 - protein, 109 aa, Artificial Sequence
  Description of Artificial Sequence: Synthetic polypeptide
EIVMTQSPATLSVSPGERATLSCRASQTINNNLAWFQQKPGQTPRLLIYGASTGATGIPARFSGSGSGTEFTLTISSLQSEDFAVYYCQQYNNWPRGLTFGGGTKVEIK

SEQ ID NO: 200 - DNA, 327 bp, Artificial Sequence
  Description of Artificial Sequence: Synthetic polynucleotide
gaaatagtgatgacgcagtctccagccaccctgtctgtgtctccaggggaaagagccaccctctcctgcagggccagtcagactattaacaacaacttagcctggttccagcagaaacctggccagactcccaggctcctcatctatggtgcatccaccggggccactggtatcccagccaggttcagtggcagtgggtctgggacagagttcactctcaccatcagcagcctgcagtctgaagattttgcagtttattactgtcagcagtataataactggcctcgaggactcactttcggcggagggaccaaggtggagatcaaa

SEQ ID NO: 201 - protein, 216 aa, Artificial Sequence
  Description of Artificial Sequence: Synthetic polypeptide
EIVMTQSPATLSVSPGERATLSCRASQTINNNLAWFQQKPGQTPRLLIYGASTGATGIPARFSGSGSGTEFTLTISSLQSEDFAVYYCQQYNNWPRGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - DNA, 648 bp, Artificial Sequence
  Description of Artificial Sequence: Synthetic polynucleotide
gaaatagtgatgacgcagtctccagccaccctgtctgtgtctccaggggaaagagccaccctctcctgcagggccagtcagactattaacaacaacttagcctggttccagcagaaacctggccagactcccaggctcctcatctatggtgcatccaccggggccactggtatcccagccaggttcagtggcagtgggtctgggacagagttcactctcaccatcagcagcctgcagtctgaagattttgcagtttattactgtcagcagtataataactggcctcgaggactcactttcggcggagggaccaaggtggagatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 203 - protein, 10 aa, Artificial Sequence
  Description of Artificial Sequence: Synthetic peptide
GYTFTGYYIN

SEQ ID NO: 204 - protein, 17 aa, Artificial Sequence
  Description of Artificial Sequence: Synthetic peptide
WINPNSGDTNYAQKFQG

SEQ ID NO: 205 - protein, 11 aa, Artificial Sequence
  Description of Artificial Sequence: Synthetic peptide
ENSGYGKLFDY

SEQ ID NO: 206 - protein, 5 aa, Artificial Sequence
  Description of Artificial Sequence: Synthetic peptide
GYYIN

SEQ ID NO: 207 - protein, 7 aa, Artificial Sequence
  Description of Artificial Sequence: Synthetic peptide
GYTFTGY

SEQ ID NO: 208 - protein, 6 aa, Artificial Sequence
  Description of Artificial Sequence: Synthetic peptide
NPNSGD

SEQ ID NO: 209 - protein, 8 aa, Artificial Sequence
  Description of Artificial Sequence: Synthetic peptide
GYTFTGYY

SEQ ID NO: 210 - protein, 8 aa, Artificial Sequence
  Description of Artificial Sequence: Synthetic peptide
INPNSGDT

SEQ ID NO: 211 - protein, 13 aa, Artificial Sequence
  Description of Artificial Sequence: Synthetic peptide
ARENSGYGKLFDY

SEQ ID NO: 212 - protein, 120 aa, Artificial Sequence
  Description of Artificial Sequence: Synthetic polypeptide
QVQLVQSGAEVKKPGASVKVSCKASGYTFTGYYINWVRQAPGQGLEWMGWINPNSGDTNYAQKFQGRVTMTRDPSISTAYMELSRLRSDDTAVYYCARENSGYGKLFDYWGQGTLVTVSS

SEQ ID NO: 213 - DNA, 360 bp, Artificial Sequence
  Description of Artificial Sequence: Synthetic polynucleotide
caggtgcagctggtgcagtctggggctgaggtgaagaagcctggggcctcagtgaaggtctcctgcaaggcttctggatacaccttcaccggctactatataaattgggtgcgacaggcccctggacaagggcttgagtggatgggatggatcaaccctaacagtggtgacacaaactatgcacagaagtttcagggcagggtcaccatgaccagggacccgtccatcagcacagcctacatggagctgagcaggctgagatctgacgacacggccgtgtattactgtgcgagagagaatagtggctacgggaagctttttgactactggggccagggaaccctggtcaccgtctcctca

SEQ ID NO: 214 - protein, 450 aa, Artificial Sequence
  Description of Artificial Sequence: Synthetic polypeptide
QVQLVQSGAEVKKPGASVKVSCKASGYTFTGYYINWVRQAPGQGLEWMGWINPNSGDTNYAQKFQGRVTMTRDPSISTAYMELSRLRSDDTAVYYCARENSGYGKLFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 215 - DNA, 1350 bp, Artificial Sequence
  Description of Artificial Sequence: Synthetic polynucleotide
caggtgcagctggtgcagtctggggctgaggtgaagaagcctggggcctcagtgaaggtctcctgcaaggcttctggatacaccttcaccggctactatataaattgggtgcgacaggcccctggacaagggcttgagtggatgggatggatcaaccctaacagtggtgacacaaactatgcacagaagtttcagggcagggtcaccatgaccagggacccgtccatcagcacagcctacatggagctgagcaggctgagatctgacgacacggccgtgtattactgtgcgagagagaatagtggctacgggaagctttttgactactggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 216 - protein, 16 aa, Artificial Sequence
  Description of Artificial Sequence: Synthetic peptide
RSSQSLLHSNGYNYLD

SEQ ID NO: 217 - protein, 7 aa, Artificial Sequence
  Description of Artificial Sequence: Synthetic peptide
LGSNRAS

SEQ ID NO: 218 - protein, 9 aa, Artificial Sequence
  Description of Artificial Sequence: Synthetic peptide
MQALQTPYT

SEQ ID NO: 219 - protein, 12 aa, Artificial Sequence
  Description of Artificial Sequence: Synthetic peptide
SQSLLHSNGYNY

SEQ ID NO: 220 - protein, 3 aa, Artificial Sequence
  Description of Artificial Sequence: Synthetic peptide
LGS

SEQ ID NO: 221 - protein, 6 aa, Artificial Sequence
  Description of Artificial Sequence: Synthetic peptide
ALQTPY

SEQ ID NO: 222 - protein, 11 aa, Artificial Sequence
  Description of Artificial Sequence: Synthetic peptide
QSLLHSNGYNY

SEQ ID NO: 223 - protein, 112 aa, Artificial Sequence
  Description of Artificial Sequence: Synthetic polypeptide
DIVMTQSPLSLPGTPGEPASISCRSSQSLLHSNGYNYLDWYLQKPGQSPQFLIYLGSNRASGVPDRFSGSGSGTDFTLKISRVEAEDVGVYYCMQALQTPYTFGQGTKLEIK

SEQ ID NO: 224 - DNA, 336 bp, Artificial Sequence
  Description of Artificial Sequence: Synthetic polynucleotide
gatattgtgatgactcagtctccactctccctgcccggcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtacacttttggccaggggaccaagctggagatcaaa

SEQ ID NO: 225 - protein, 219 aa, Artificial Sequence
  Description of Artificial Sequence: Synthetic polypeptide
DIVMTQSPLSLPGTPGEPASISCRSSQSLLHSNGYNYLDWYLQKPGQSPQFLIYLGSNRASGVPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - DNA, 657 bp, Artificial Sequence
  Description of Artificial Sequence: Synthetic polynucleotide
gatattgtgatgactcagtctccactctccctgcccggcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtacacttttggccaggggaccaagctggagatcaaacgaactgtggctgcaccaagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 227 - protein, 10 aa, Artificial Sequence
  Description of Artificial Sequence: Synthetic peptide
GGSISSYYWT

SEQ ID NO: 228 - protein, 16 aa, Artificial Sequence
  Description of Artificial Sequence: Synthetic peptide
RVFTSESTNYNPSLKN

SEQ ID NO: 229 - protein, 11 aa, Artificial Sequence
  Description of Artificial Sequence: Synthetic peptide
DRGTYLGGFDP

SEQ ID NO: 230 - protein, 5 aa, Artificial Sequence
  Description of Artificial Sequence: Synthetic peptide
SYYWT

SEQ ID NO: 231 - protein, 5 aa, Artificial Sequence
  Description of Artificial Sequence: Synthetic peptide
FTSES

SEQ ID NO: 232 - protein, 7 aa, Artificial Sequence
  Description of Artificial Sequence: Synthetic peptide
VFTSEST

SEQ ID NO: 233 - protein, 13 aa, Artificial Sequence
  Description of Artificial Sequence: Synthetic peptide
ARDRGTYLGGFDP

SEQ ID NO: 234 - protein, 119 aa, Artificial Sequence
  Description of Artificial Sequence: Synthetic polypeptide
QVQLQESGPGLVKPSETLSLTCTVSGGSISSYYWTWIRQPAGKGLEWIGRVFTSESTNYNPSLKNRVTMSVDTSKNQFSLRLNSVTAADTAMYYCARDRGTYLGGFDPWGQGTLVTVSS

SEQ ID NO: 235 - DNA, 357 bp, Artificial Sequence
  Description of Artificial Sequence: Synthetic polynucleotide
caggtgcagctgcaggagtcgggcccaggactggtgaagccttcggagaccctgtccctcacctgcactgtctctggtggctccatcagtagttactactggacctggatccggcagcccgccgggaagggactggagtggattgggcgtgtctttaccagtgagagcaccaactacaacccctccctcaagaatcgagtcaccatgtcagtagacacgtccaagaaccagttctccctgaggctgaattctgtgaccgccgcggacacggccatgtattactgtgcgagagaccgggggacctacctaggggggttcgacccctggggccagggaaccctggtcaccgtctcctca

SEQ ID NO: 236 - protein, 449 aa, Artificial Sequence
  Description of Artificial Sequence: Synthetic polypeptide
QVQLQESGPGLVKPSETLSLTCTVSGGSISSYYWTWIRQPAGKGLEWIGRVFTSESTNYNPSLKNRVTMSVDTSKNQFSLRLNSVTAADTAMYYCARDRGTYLGGFDPWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 237 - DNA, 1347 bp, Artificial Sequence
  Description of Artificial Sequence: Synthetic polynucleotide
caggtgcagctgcaggagtcgggcccaggactggtgaagccttcggagaccctgtccctcacctgcactgtctctggtggctccatcagtagttactactggacctggatccggcagcccgccgggaagggactggagtggattgggcgtgtctttaccagtgagagcaccaactacaacccctccctcaagaatcgagtcaccatgtcagtagacacgtccaagaaccagttctccctgaggctgaattctgtgaccgccgcggacacggccatgtattactgtgcgagagaccgggggacctacctaggggggttcgacccctggggccagggaaccctggtcaccgtctcctcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 238 - protein, 9 aa, Artificial Sequence
  Description of Artificial Sequence: Synthetic peptide
IQALQTPFT

SEQ ID NO: 239 - protein, 6 aa, Artificial Sequence
  Description of Artificial Sequence: Synthetic peptide
ALQTPF

SEQ ID NO: 240 - protein, 112 aa, Artificial Sequence
  Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCIQALQTPFTFGQGTKLEIK

SEQ ID NO: 241 - DNA, 336 bp, Artificial Sequence
  Description of Artificial Sequence: Synthetic polynucleotide
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatacaagctctacaaactccgttcacttttggccaggggaccaaactggagatcaaa

SEQ ID NO: 242 - protein, 219 aa, Artificial Sequence
  Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCIQALQTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - DNA, 657 bp, Artificial Sequence
  Description of Artificial Sequence: Synthetic polynucleotide
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatacaagctctacaaactccgttcacttttggccaggggaccaaactggagatcaaacgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 244 - protein, 12 aa, Artificial Sequence
  Description of Artificial Sequence: Synthetic peptide
GFSLSTSGMSVS

SEQ ID NO: 245 - protein, 9 aa, Artificial Sequence
  Description of Artificial Sequence: Synthetic peptide
MALRHAFDA

SEQ ID NO: 246 - protein, 9 aa, Artificial Sequence
  Description of Artificial Sequence: Synthetic peptide
GFSLSTSGM

SEQ ID NO: 247 - protein, 10 aa, Artificial Sequence
  Description of Artificial Sequence: Synthetic peptide
GFSLSTSGMS

SEQ ID NO: 248 - protein, 11 aa, Artificial Sequence
  Description of Artificial Sequence: Synthetic peptide
ARMALRHAFDA

SEQ ID NO: 249 - protein, 119 aa, Artificial Sequence
  Description of Artificial Sequence: Synthetic polypeptide
QVTLRESGPALVKPTQTLTLTCTFSGFSLSTSGMSVSWIRQPPGKALEWLALIDWDDDKYYSTSLKTRLTISKDTSKNQVVLTMTNMDPVDTATYYCARMALRHAFDAWGQGTMVTVSS

SEQ ID NO: 250 - DNA, 357 bp, Artificial Sequence
  Description of Artificial Sequence: Synthetic polynucleotide
caggtcaccttgagggagtctggtcctgcgctggtgaagcccacacagaccctcacactgacctgcaccttctctgggttctcactcagcactagtggaatgtctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggatggcactacgtcatgcttttgatgcctggggccaagggacaatggtcaccgtctcttca

SEQ ID NO: 251 - protein, 449 aa, Artificial Sequence
  Description of Artificial Sequence: Synthetic polypeptide
QVTLRESGPALVKPTQTLTLTCTFSGFSLSTSGMSVSWIRQPPGKALEWLALIDWDDDKYYSTSLKTRLTISKDTSKNQVVLTMTNMDPVDTATYYCARMALRHAFDAWGQGTMVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 252 - DNA, 1347 bp, Artificial Sequence
  Description of Artificial Sequence: Synthetic polynucleotide
caggtcaccttgagggagtctggtcctgcgctggtgaagcccacacagaccctcacactgacctgcaccttctctgggttctcactcagcactagtggaatgtctgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggatggcactacgtcatgcttttgatgcctggggccaagggacaatggtcaccgtctcttcagcctccaccaagggcccatcggtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 253 - protein, 14 aa, Artificial Sequence
  Description of Artificial Sequence: Synthetic peptide
TGSSSNIGAGYDVH

SEQ ID NO: 254 - protein, 7 aa, Artificial Sequence
  Description of Artificial Sequence: Synthetic peptide
VNSNRPS

SEQ ID NO: 255 - protein, 11 aa, Artificial Sequence
  Description of Artificial Sequence: Synthetic peptide
QSYDSSLSGWV

SEQ ID NO: 256 - protein, 10 aa, Artificial Sequence
  Description of Artificial Sequence: Synthetic peptide
SSSNIGAGYD

SEQ ID NO: 257 - protein, 3 aa, Artificial Sequence
  Description of Artificial Sequence: Synthetic peptide
VNS

SEQ ID NO: 258 - protein, 8 aa, Artificial Sequence
  Description of Artificial Sequence: Synthetic peptide
YDSSLSGW

SEQ ID NO: 259 - protein, 9 aa, Artificial Sequence
  Description of Artificial Sequence: Synthetic peptide
SSNIGAGYD

SEQ ID NO: 260 - protein, 111 aa, Artificial Sequence
  Description of Artificial Sequence: Synthetic polypeptide
QSVLTQPPSVSGAPGQRVTISCTGSSSNIGAGYDVHWYQQLPGTAPKLLIYVNSNRPSGVPDRFSGSKSGTSASLAITGLQAEDEADYYCQSYDSSLSGWVFGGGTKLTVL

SEQ ID NO: 261 - DNA, 333 bp, Artificial Sequence
  Description of Artificial Sequence: Synthetic polynucleotide
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatgttaacagcaatcggccctcaggggtccctgaccgattctctggctccaagtctggcacctcagcctccctggccatcactgggctccaggctgaggatgaggctgattattactgccagtcctatgacagcagcctgagtggttgggtgttcggcggagggaccaagttgaccgtccta

SEQ ID NO: 262 - protein, 217 aa, Artificial Sequence
  Description of Artificial Sequence: Synthetic polypeptide
QSVLTQPPSVSGAPGQRVTISCTGSSSNIGAGYDVHWYQQLPGTAPKLLIYVNSNRPSGVPDRFSGSKSGTSASLAITGLQAEDEADYYCQSYDSSLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 263 - DNA, 651 bp, Artificial Sequence
  Description of Artificial Sequence: Synthetic polynucleotide
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatgttaacagcaatcggccctcaggggtccctgaccgattctctggctccaagtctggcacctcagcctccctggccatcactgggctccaggctgaggatgaggctgattattactgccagtcctatgacagcagcctgagtggttgggtgttcggcggagggaccaagttgaccgtcctaggtcagcccaaggctgccccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 264 - protein, 10 aa, Artificial Sequence
  Description of Artificial Sequence: Synthetic peptide
GFSLTNYGVH

SEQ ID NO: 265 - protein, 16 aa, Artificial Sequence
  Description of Artificial Sequence: Synthetic peptide
VIWRGESTDYNAAFMS

SEQ ID NO: 266 - protein, 11 aa, Artificial Sequence
  Description of Artificial Sequence: Synthetic peptide
NGGSSGWYFDV

SEQ ID NO: 267 - protein, 5 aa, Artificial Sequence
  Description of Artificial Sequence: Synthetic peptide
NYGVH

SEQ ID NO: 268 - protein, 7 aa, Artificial Sequence
  Description of Artificial Sequence: Synthetic peptide
GFSLTNY

SEQ ID NO: 269 - protein, 5 aa, Artificial Sequence
  Description of Artificial Sequence: Synthetic peptide
WRGES

SEQ ID NO: 270 - protein, 8 aa, Artificial Sequence
  Description of Artificial Sequence: Synthetic peptide
GFSLTNYG

SEQ ID NO: 271 - protein, 7 aa, Artificial Sequence
  Description of Artificial Sequence: Synthetic peptide
IWRGEST

SEQ ID NO: 272 - protein, 13 aa, Artificial Sequence
  Description of Artificial Sequence: Synthetic peptide
ARNGGSSGWYFDV

SEQ ID NO: 273 - protein, 119 aa, Artificial Sequence
  Description of Artificial Sequence: Synthetic polypeptide
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNGGSSGWYFDVWGQGTTVTVSS

SEQ ID NO: 274 - DNA, 357 bp, Artificial Sequence
  Description of Artificial Sequence: Synthetic polynucleotide
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacggcggatcctccggctggtactttgatgtgtggggccagggcaccaccgtgacagttagttct

SEQ ID NO: 275 - protein, 449 aa, Artificial Sequence
  Description of Artificial Sequence: Synthetic polypeptide
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNGGSSGWYFDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 276 - DNA, 1347 bp, Artificial Sequence
  Description of Artificial Sequence: Synthetic polynucleotide
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacggcggatcctccggctggtactttgatgtgtggggccagggcaccaccgtgacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 277 - protein, 11 aa, Artificial Sequence
  Description of Artificial Sequence: Synthetic peptide
KASQDVTSAVA

SEQ ID NO: 278 - protein, 7 aa, Artificial Sequence
  Description of Artificial Sequence: Synthetic peptide
WTSTRHT

SEQ ID NO: 279 - protein, 9 aa, Artificial Sequence
  Description of Artificial Sequence: Synthetic peptide
QQHYTTPLT

SEQ ID NO: 280 - protein, 7 aa, Artificial Sequence
  Description of Artificial Sequence: Synthetic peptide
SQDVTSA

SEQ ID NO: 281 - protein, 3 aa, Artificial Sequence
  Description of Artificial Sequence: Synthetic peptide
WTS

SEQ ID NO: 282 - protein, 6 aa, Artificial Sequence
  Description of Artificial Sequence: Synthetic peptide
HYTTPL

SEQ ID NO: 283 - protein, 6 aa, Artificial Sequence
  Description of Artificial Sequence: Synthetic peptide
QDVTSA

SEQ ID NO: 284 - protein, 107 aa, Artificial Sequence
  Description of Artificial Sequence: Synthetic polypeptide
DIQLTQSPSFLSASVGDRVTITCKASQDVTSAVAWYQQKPGKAPKLLIYWTSTRHTGVPSRFSGSGSGTEYTLTISSLQPEDFATYYCQQHYTTPLTFGQGTKLEIK

SEQ ID NO: 285 - DNA, 321 bp, Artificial Sequence
  Description of Artificial Sequence: Synthetic polynucleotide
gatattcagctgacccagtctcctagcttcctgtccgcttctgtgggcgacagagtgaccatcacatgcaaggcctctcaggacgtgacctctgccgtggcttggtatcagcagaagcctggcaaggcccctaagctgctgatctactggacctccaccagacacaccggcgtgccctctagattttccggctctggctctggcaccgagtataccctgacaatctccagcctgcagcctgaggacttcgccacctactactgccagcagcactacaccacacctctgacctttggccagggcaccaagctggaaatcaag

SEQ ID NO: 286 - protein, 214 aa, Artificial Sequence
  Description of Artificial Sequence: Synthetic polypeptide
DIQLTQSPSFLSASVGDRVTITCKASQDVTSAVAWYQQKPGKAPKLLIYWTSTRHTGVPSRFSGSGSGTEYTLTISSLQPEDFATYYCQQHYTTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - DNA, 642 bp, Artificial Sequence
  Description of Artificial Sequence: Synthetic polynucleotide
gatattcagctgacccagtctcctagcttcctgtccgcttctgtgggcgacagagtgaccatcacatgcaaggcctctcaggacgtgacctctgccgtggcttggtatcagcagaagcctggcaaggcccctaagctgctgatctactggacctccaccagacacaccggcgtgccctctagattttccggctctggctctggcaccgagtataccctgacaatctccagcctgcagcctgaggacttcgccacctactactgccagcagcactacaccacacctctgacctttggccagggcaccaagctggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 288 - protein, 119 aa, Artificial Sequence
  Description of Artificial Sequence: Synthetic polypeptide
QVQLQQSGPGLVQPSQSLSITCTVSGFSLTNYGVHWVRQSPGKGLEWLGVIWRGESTDYNAAFMSRLSVTKDDSKSQVFFKMNSLQADDTAIYYCARNGGSSGWYFDVWGTGTTVTVSS

SEQ ID NO: 289 - DNA, 357 bp, Artificial Sequence
  Description of Artificial Sequence: Synthetic polynucleotide
caggtgcagctacagcagtcaggacctggcctagtgcagccctcacagagcctgtccataacctgcacagtctctggtttctcattaactaactatggtgtacactgggttcgccagtctccaggaaagggtctggagtggctgggagtgatatggagaggtgaaagcacagactacaatgcagctttcatgtccagactgagcgtcaccaaggacgactccaagagccaagttttctttaaaatgaacagtctgcaagctgatgacactgccatatactactgtgccagaaatggcggtagtagcgggtggtacttcgatgtctggggcacagggaccactgtcaccgtctcctca

SEQ ID NO: 290 - protein, 454 aa, Artificial Sequence
  Description of Artificial Sequence: Synthetic polypeptide
QVQLQQSGPGLVQPSQSLSITCTVSGFSLTNYGVHWVRQSPGKGLEWLGVIWRGESTDYNAAFMSRLSVTKDDSKSQVFFKMNSLQADDTAIYYCARNGGSSGWYFDVWGTGTTVTVSSAKTTAPSVYPLAPVCGGTTGSSVTLGCLVKGYFPCPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALASPIEKTISKPRGPVRAPQVYVLPPPAEEMTKKEFSLTCMITGFLPCEIAVDWTSNGRTEQNYKNTATVLDSDGSYFMYSKLRVQKSTWERGSLFACSVVHEGLHNHLTTKTISRSLGK

SEQ ID NO: 291 - DNA, 1362 bp, Artificial Sequence
  Description of Artificial Sequence: Synthetic polynucleotide
caggtgcagctacagcagtcaggacctggcctagtgcagccctcacagagcctgtccataacctgcacagtctctggtttctcattaactaactatggtgtacactgggttcgccagtctccaggaaagggtctggagtggctgggagtgatatggagaggtgaaagcacagactacaatgcagctttcatgtccagactgagcgtcaccaaggacgactccaagagccaagttttctttaaaatgaacagtctgcaagctgatgacactgccatatactactgtgccagaaatggcggtagtagcgggtggtacttcgatgtctggggcacagggaccactgtcaccgtctcctcagccaaaacaacagccccatcggtctatccactggcccctgtgtgtggaggtacaactggctcctcggtgactctaggatgcctggtcaagggttatttcccttgtccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggctgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcgcatcccccatcgagaaaaccatctcaaaacccagagggccagtaagagctccacaggtatatgtcttgcctccaccagcagaagagatgactaagaaagagttcagtctgacctgcatgatcacaggcttcttaccttgtgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 292 - protein, 107 aa, Artificial Sequence
  Description of Artificial Sequence: Synthetic polypeptide
DIQMTQTHKFMSTSVGDRVSITCKASQDVTSAVAWYQQTPGQSPNLLIYWTSTRHTGVPDRFTGSGSGTDYTLTISSVQAEDLALYYCQQHYTTPLTFGAGTKLELK

SEQ ID NO: 293 - DNA, 321 bp, Artificial Sequence
  Description of Artificial Sequence: Synthetic polynucleotide
gacattcagatgacccagactcacaaattcatgtccacatcagtaggagacagggtcagcatcacctgcaaggccagtcaggatgtgacttctgctgtagcctggtatcaacaaacaccaggacaatctcctaatctactgatttactggacatccacccggcacactggagtccctgatcgcttcacaggcagtggatctgggacagattatactctcaccatcagcagtgtgcaggctgaagacctggcactttattactgtcagcaacattataccactccgctcacgttcggtgctgggaccaagctggagctaaaa

SEQ ID NO: 294 - protein, 214 aa, Artificial Sequence
  Description of Artificial Sequence: Synthetic polypeptide
DIQMTQTHKFMSTSVGDRVSITCKASQDVTSAVAWYQQTPGQSPNLLIYWTSTRHTGVPDRFTGSGSGTDYTLTISSVQAEDLALYYCQQHYTTPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 295 - DNA, 642 bp, Artificial Sequence
  Description of Artificial Sequence: Synthetic polynucleotide
gacattcagatgacccagactcacaaattcatgtccacatcagtaggagacagggtcagcatcacctgcaaggccagtcaggatgtgacttctgctgtagcctggtatcaacaaacaccaggacaatctcctaatctactgatttactggacatccacccggcacactggagtccctgatcgcttcacaggcagtggatctgggacagattatactctcaccatcagcagtgtgcaggctgaagacctggcactttattactgtcagcaacattataccactccgctcacgttcggtgctgggaccaagctggagctaaaacgtgccgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 296 - protein, 11 aa, Artificial Sequence
  Description of Artificial Sequence: Synthetic peptide
NAGSSGWYFDV

SEQ ID NO: 297 - protein, 13 aa, Artificial Sequence
  Description of Artificial Sequence: Synthetic peptide
ARNAGSSGWYFDV

SEQ ID NO: 298 - protein, 119 aa, Artificial Sequence
  Description of Artificial Sequence: Synthetic polypeptide
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNAGSSGWYFDVWGQGTTVTVSS

SEQ ID NO: 299 - DNA, 357 bp, Artificial Sequence
  Description of Artificial Sequence: Synthetic polynucleotide
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacgctggctcctccggctggtactttgatgtgtggggccagggcaccaccgtgacagttagttct

SEQ ID NO: 300 - protein, 449 aa, Artificial Sequence
  Description of Artificial Sequence: Synthetic polypeptide
QVQLQESGPGLVKPSETLSLTCTVSGFSLTNYGVHWIRQPPGKGLEWIGVIWRGESTDYNAAFMSRVTISKDDSKSQVSLKLSSVTAADTAVYYCARNAGSSGWYFDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 301 - DNA, 1347 bp, Artificial Sequence
  Description of Artificial Sequence: Synthetic polynucleotide
caagttcagctgcaagaatctggccctggcctggtcaagccttccgagacactgtctctgacctgcaccgtgtctggcttctccctgaccaattacggcgtgcactggatcagacagcctccaggcaaaggcctggaatggatcggagtgatttggagaggcgagtccaccgactacaacgccgccttcatgtccagagtgaccatctccaaggacgactccaagagccaggtgtccctgaagctgtcctctgtgaccgctgctgataccgccgtgtactactgtgccagaaacgctggctcctccggctggtactttgatgtgtggggccagggcaccaccgtgacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 302 - DNA, 4328 bp, Homo sapiens
atcacagggtgggaaataaaagctgtggcccccaggagttctggacactgggggagagtggggtgacatgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtagcagaacttcacccccttttaagctacagttccttctctccatccttcgaccttcacaaaatctctgggactgttctttgtcagattcttcctcctttagaaggctgggtcccattctgtccttcttgtcatgcctccaatttcccctggtgtagagcttgtttttctggcccatccttggagctttatgagtgagctggtgtgggatgcctttgggggtggacttgtgttccaagaatccactctctcttccttttggagattaggatatttgggttgccatgtgtagctgctatgtcccctggggcgttatcttatacatgcaaacctaccatctgttcaacttccacctaccacctcctgcacccctttgatcggggacttactggttgcaagagctcattttgcaggctggaagcaccagggaattaattcccccagtcaaccaatggcacccagagagggcatggaggctccacgcaaccccttccacccccacatcttcctttgtcttatacatggcttccatttggctgtttctaagttgtattctttattttattattattattactatttttcgagatggagtttcactcttgtcgctcaggctggagtgccatggcgcgatcttggctcactgcaacctctgcctcccgggttcaagtgattctcctgcctcagcctcacgagtagctggaattacaggcaggcgccaccagacccggctaattttttgtatttttagtacagatggggtttctccgtgttggtcaggctggtcttgaactcccgacctcagatgatctgcccgcctcggcctcccaaaattgctgggattacaggtgtgagccaccgcgcctggcctattattttttgtaagaataaaacaggtttattgggatttgggactctgaacagttctgtctctactacctgatctcctcctaccacgactttgggatctagaggagctttggctccggctgtgacggctccggccgttctcactgcggctgcaccggcccccgctgcggtcactatttcttcctctgctaggtgaattgtgcctctcctggctctttgacatgtgctagtgagatttcttccttttcctttcggattccccatttcttttgtaggaatggtctggactagggttctccttccccgcagcctgtagtattcatcgtggtggcccaccctctctctccccttggagctcttgccaaaggaggagacaagcagaggtctctattggatttctcaacacctgaagaaagttgcagtgttttcctcttggacattgttgtatttcaaataaaccacaaatcatcattttccaccgagccactgggcagaattcacactgaagctgtcgtcctgcgtacataccatcgtccgttaaacagagaaagagctgcttggcattcttcttccgactggtactgaacatatatacttgcccctcaggtgaggttccaagttgcaactgaccttgaactgaatcactctccccacgttattttttaattactatttttttttaaagatggggtcttgctctgtcgccaggctggagtgcagtggcgcgatctaggctcactgcaacttccgcctcccgggttcaagcgattctcctgcctcagcctcccgagtagctgggactccactaaaagtacaaaaattagctgggcgtgcaccactgcgcccagctaattcttgtatttttggtagagacggggtttcaacatgttgaccaggatggtctcgatctcttgacctcgtgattcgcccgccgcgtcctcccaaagtgctgggattacaggcctgagccaccgcgcccagtctctccccacgttcttgaactcgggcagcacatcctcacagaaatctaggaactgttggtaggtttcttcctcgctgtactccaggcttgcttcggagtcatagtcatccctcctgcactgctcctttccaaacactgtaaacatgcttttaataagaagggtaggactggatgttgggaaatcatgtgaacatctatctccaaatctgcaagctcctgttttactgtagaagggacaattaactccatccttctccatgactctgaaatccaagggggggttccgggttttgccatgtggcgccattttccaactcattttcagcctgatccagcatcttctggacagcttccggtttttgtttcttctgtcgtttctgttcctcctcctctctctctttcctctgctgttcttcccattgttcctttaactttcgctcttgttcttgccgttttctagccacctcttccttttccttctttattctgaattcttcttgtgccttctgctctctcagcaaccactcctcatgtaatctttgcctctctcttccccatagcttttctagttgttgtttttcaataaaagtgtcctcctctttctgtgagagtcctgagtccctcagtggagcaagttcctgctggcgtttctttcgtttctccttcttcagggcggccctgtactttttgtggcttggtttctctggaaatgtcaccttttcgggcgcagccatcttgccggcaccgccccgcccctctagttgtatcctttataataaactggtaaacattgtaaccgcagattcagcccaatctggttcaactttgtgtaataaaatggcgagttgtttttcagttgtcgtggacccccaggttgcaagttacataccctgggcatgtccagatgaacgaagcgtgcaaatccacgtggaacctaagtgctcagaccgaggaacagggactgagttaagaagtggacaccacgtggcatgatccttgatccaatcagattgagccctggcgtgatccagtcagatcaagcctcctgaatcccctcattacaagatccaatcatatcatgcctcactaccctctgtatataaaatctgccccagcctccaacttggagagacagatttgggccagactcctgtgtccttgcttggctgccttgcaataaatttttctctctacaaaa

SEQ ID NO: 303 - protein, 404 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 304 - DNA, 1976 bp, Homo sapiens
acccagcttcctgtttgtcttcctgagagacagtagatttagaaagtgaggatcagagggtggaaaataaaagctgtggtccccaggagtcctgaacatctggggacagcgggaaaacatgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatagttgtttccctgctagcctcagcctccattgtggtatagcagaacttcacccacttgtaagccagcgcttcttctctccatccttggaccttcacaaatgccctgagacggttctctgttcgatttttcatcccctatgaacctgggtcttattctgtccttctgatgcctccaagtttccctggtgtagagcttgtgttcttggcccatccttggagctttataagtgacctgagtgggatgcatttagggggcgggcttggtatgttgtatgaatccactctctgttccttttggagattagactatttggattcatgtgtagctgccctgtcccctggggctttatctcatccatgcaaactaccatctgctcaacttccagctacaccccgtgcacccttttgactggggacttgctggttgaaggagctcatcttgcaggctggaagcaccagggaattaattcccccagtcaaccaatggcatccagagagggcatggaggctccatacaacctcttccacccccacatctttctttgtcctatacatgtcttccatttggctgtttctgagttgtagcctttataataaagtggtaaatgttgtaactgcaaaaaaaaaaaaaaaaaaaaaaaaaaaaaaaaaaaaaaaaaa

SEQ ID NO: 305 - protein, 399 aa, Artificial Sequence
  Description of Artificial Sequence: Synthetic polypeptide
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 306 - DNA, 1212 bp, Artificial Sequence
  Description of Artificial Sequence: Synthetic polynucleotide
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcg

SEQ ID NO: 307 - protein, 343 aa, Artificial Sequence
  Description of Artificial Sequence: Synthetic polypeptide
KVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 308 - DNA, 1029 bp, Artificial Sequence
  Description of Artificial Sequence: Synthetic polynucleotide
aaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcg

SEQ ID NO: 309 - protein, 155 aa, Artificial Sequence
  Description of Artificial Sequence: Synthetic polypeptide
ERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 310 - DNA, 465 bp, Artificial Sequence
  Description of Artificial Sequence: Synthetic polynucleotide
gaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaaccctcctcctgcg

SEQ ID NO: 311 - protein, 406 aa, Artificial Sequence
  Description of Artificial Sequence: Synthetic polypeptide
MSDSKEPRLQQLDLLEEEQLGGVGFRQTRGYKSLAGCLGHGPLVLQLLSFTLLAGLLVQVSKVPSSLSQGQSKQDAIYQNLTQLKVAVSELSEKSKQQEIYQELTRLKAAVGELPEKSKQQEIYEELTRLKAAVGELPEKSKLQEIYQELTRLKAAVGELPEKSKQQEIYQELSRLKAAVGDLPEKSKQQEIYQKLTQLKAAVDGLPDRSKQQEIYQELIQLKAAVDLEGWTDTGIWTTSSEPSPDRPPPTERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPPE

SEQ ID NO: 312 - DNA, 1224 bp, Artificial Sequence
  Description of Artificial Sequence: Synthetic polynucleotide
atgtcggactcgaaggaaccaagactgcagcaactcgacctccttgaagaagaacagctcggcggagtgggattccggcagaccaggggttacaagagcctggccggttgcctgggtcacggccctttggtgcttcagctgctgtcgttcaccctgctggccggactgcttgtgcaagtctccaaagtcccgtcctcgctgagccaggggcagtccaagcaggacgcgatctaccaaaacctgacacagctcaaggtggccgtgtcagagctgtccgagaagtcgaagcagcaagagatctaccaagagttgacgcgactcaaagcagccgtgggcgaacttcccgagaagtcaaagcagcaggaaatctacgaggaattgacccgcctgaaggccgccgtgggagagctgccagaaaagtcgaagctgcaggagatataccaagaactcacccggctcaaggccgctgtgggagaactgccggagaagtccaaacaacaggaaatctaccaggaactgagcagactcaaggcagccgtcggcgatctccccgaaaagtctaaacagcaggagatctatcagaagctgactcagctgaaggcggccgtggacgggctgcccgatcggtccaagcaacaggaaatctaccaggagctgatccaactgaaggctgccgtggacctggaagggtggactgacaccgggatttggactacctcatcggaaccgagccctgatcgccctccgcctaccgagaggttgtgtcacccgtgcccatgggagtggacgttcttccaaggaaactgttactttatgagcaacagccagcggaattggcacgattccattaccgcgtgccaggaagtgggcgcccagctggtcgtgatcaagtccgcggaggagcagaacttcctgcagctccagagcagccggtccaaccgcttcacctggatgggcctctccgacctgaaccatgagggaacttggcagtgggtggacggttccccgctgctgccctcattcaagcagtactggaacaagggagaaccgaacaacgtcggagaggaagattgcgccgagttttccgggaacggatggaacgacgacaagtgcaatctggccaagttctggatttgcaagaagtccgctgcatcctgctcgggcgacgaggagcgcctgctgtcccccgcgcccaccacccctaaccctcccccggaatgatag

SEQ ID NO: 313 - protein, 336 aa, Artificial Sequence
  Description of Artificial Sequence: Synthetic polypeptide
QPSKQDAIYQNLTQLKVAVSELSEKSKQQEIYQELTRLKAAVGELPEKSKQQEIYEELTRLKAAVGELPEKSKLQEIYQELTRLKAAVGELPEKSKQQEIYQELSRLKAAVGDLPEKSKQQEIYQKLTQLKAAVDGLPDRSKQQEIYQELIQLKAAVDLEGWTDTGIWTTSSEPSPDRPPPTERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPP

SEQ ID NO: 314 - DNA, 1005 bp, Artificial Sequence
  Description of Artificial Sequence: Synthetic polynucleotide
tccaagcaggacgcgatctaccaaaacctgacacagctcaaggtggccgtgtcagagctgtccgagaagtcgaagcagcaagagatctaccaagagttgacgcgactcaaagcagccgtgggcgaacttcccgagaagtcaaagcagcaggaaatctacgaggaattgacccgcctgaaggccgccgtgggagagctgccagaaaagtcgaagctgcaggagatataccaagaactcacccggctcaaggccgctgtgggagaactgccggagaagtccaaacaacaggaaatctaccaggaactgagcagactcaaggcagccgtcggcgatctccccgaaaagtctaaacagcaggagatctatcagaagctgactcagctgaaggcggccgtggacgggctgcccgatcggtccaagcaacaggaaatctaccaggagctgatccaactgaaggctgccgtggacctggaagggtggactgacaccgggatttggactacctcatcggaaccgagccctgatcgccctccgcctaccgagaggttgtgtcacccgtgcccatgggagtggacgttcttccaaggaaactgttactttatgagcaacagccagcggaattggcacgattccattaccgcgtgccaggaagtgggcgcccagctggtcgtgatcaagtccgcggaggagcagaacttcctgcagctccagagcagccggtccaaccgcttcacctggatgggcctctccgacctgaaccatgagggaacttggcagtgggtggacggttccccgctgctgccctcattcaagcagtactggaacaagggagaaccgaacaacgtcggagaggaagattgcgccgagttttccgggaacggatggaacgacgacaagtgcaatctggccaagttctggatttgcaagaagtccgctgcatcctgctcgggcgacgaggagcgcctgctgtcccccgcgcccaccacccctaaccctcccccggaa

SEQ ID NO: 315 - protein, 157 aa, Artificial Sequence
  Description of Artificial Sequence: Synthetic polypeptide
QPERLCHPCPWEWTFFQGNCYFMSNSQRNWHDSITACQEVGAQLVVIKSAEEQNFLQLQSSRSNRFTWMGLSDLNHEGTWQWVDGSPLLPSFKQYWNKGEPNNVGEEDCAEFSGNGWNDDKCNLAKFWICKKSAASCSGDEERLLSPAPTTPNPPPE

SEQ ID NO: 316 - DNA, 465 bp, Artificial Sequence
  Description of Artificial Sequence: Synthetic polynucleotide
gagaggttgtgtcacccgtgcccatgggagtggacgttcttccaaggaaactgttactttatgagcaacagccagcggaattggcacgattccattaccgcgtgccaggaagtgggcgcccagctggtcgtgatcaagtccgcggaggagcagaacttcctgcagctccagagcagccggtccaaccgcttcacctggatgggcctctccgacctgaaccatgagggaacttggcagtgggtggacggttccccgctgctgccctcattcaagcagtactggaacaagggagaaccgaacaacgtcggagaggaagattgcgccgagttttccgggaacggatggaacgacgacaagtgcaatctggccaagttctggatttgcaagaagtccgctgcatcctgctcgggcgacgaggagcgcctgctgtcccccgcgcccaccacccctaaccctcccccggaa

SEQ ID NO: 317 - protein, 368 aa, Artificial Sequence
  Description of Artificial Sequence: Synthetic polypeptide
KVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPAGSGGGLNDIFEAQKIEWHEHHHHHH

SEQ ID NO: 318 - protein, 198 aa, Artificial Sequence
  Description of Artificial Sequence: Synthetic polypeptide
MQLLSCIALSLALVTNSTERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPAGSGGGLNDIFEAQKIEWHEHHHHHH

SEQ ID NO: 319 - protein, 384 aa, Artificial Sequence
  Description of Artificial Sequence: Synthetic polypeptide
MKTFILLLWVLLLWVIFLLPGATAQPSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPADYKDDDDKHHHHHH

SEQ ID NO: 320 - protein, 11 aa, Artificial Sequence
  Description of Artificial Sequence: Synthetic peptide
VVIKSAEEQNF

SEQ ID NO: 321 - protein, 19 aa, Artificial Sequence
  Description of Artificial Sequence: Synthetic peptide
LQLQSSRSNRFTWMGLSDL

SEQ ID NO: 322 - protein, 15 aa, Artificial Sequence
  Description of Artificial Sequence: Synthetic peptide
NQEGTWQWVDGSPLL

SEQ ID NO: 323 - protein, 18 aa, Artificial Sequence
  Description of Artificial Sequence: Synthetic peptide
NQEGTWQWVDGSPLLPSF

SEQ ID NO: 324-500 - skipped (177 entries)

SEQ ID NO: 501 - protein, 5 aa, Artificial Sequence
  Description of Artificial Sequence: Synthetic peptide
TYWMH

SEQ ID NO: 502 - protein, 17 aa, Artificial Sequence
  Description of Artificial Sequence: Synthetic peptide
NIYPGTGGSNFDEKFKN

SEQ ID NO: 503 - protein, 8 aa, Artificial Sequence
  Description of Artificial Sequence: Synthetic peptide
WTTGTGAY

SEQ ID NO: 504 - protein, 7 aa, Artificial Sequence
  Description of Artificial Sequence: Synthetic peptide
GYTFTTY

SEQ ID NO: 505 - protein, 6 aa, Artificial Sequence
  Description of Artificial Sequence: Synthetic peptide
YPGTGG

SEQ ID NO: 506 - protein, 117 aa, Artificial Sequence
  Description of Artificial Sequence: Synthetic polypeptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  Description of Artificial Sequence: Synthetic polynucleotide
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  Description of Artificial Sequence: Synthetic polypeptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  Description of Artificial Sequence: Synthetic polynucleotide
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  Description of Artificial Sequence: Synthetic peptide
KSSQSLLDSGNQKNFLT

SEQ ID NO: 511 - protein, 7 aa, Artificial Sequence
  Description of Artificial Sequence: Synthetic peptide
WASTRES

SEQ ID NO: 512 - protein, 9 aa, Artificial Sequence
  Description of Artificial Sequence: Synthetic peptide
QNDYSYPYT

SEQ ID NO: 513 - protein, 13 aa, Artificial Sequence
  Description of Artificial Sequence: Synthetic peptide
SQSLLDSGNQKNF

SEQ ID NO: 514 - protein, 3 aa, Artificial Sequence
  Description of Artificial Sequence: Synthetic peptide
WAS

SEQ ID NO: 515 - protein, 6 aa, Artificial Sequence
  Description of Artificial Sequence: Synthetic peptide
DYSYPY

SEQ ID NO: 516 - protein, 113 aa, Artificial Sequence
  Description of Artificial Sequence: Synthetic polypeptide
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  Description of Artificial Sequence: Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  Description of Artificial Sequence: Synthetic polypeptide
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  Description of Artificial Sequence: Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  Description of Artificial Sequence: Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  Description of Artificial Sequence: Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  Description of Artificial Sequence: Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  Description of Artificial Sequence: Synthetic polynucleotide
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  Description of Artificial Sequence: Synthetic oligonucleotide
acctactggatgcac

SEQ ID NO: 525 - DNA, 51 bp, Artificial Sequence
  Description of Artificial Sequence: Synthetic oligonucleotide
aatatctaccccggcaccggcggctctaacttcgacgagaagtttaagaat

SEQ ID NO: 526 - DNA, 24 bp, Artificial Sequence
  Description of Artificial Sequence: Synthetic oligonucleotide
tggactaccggcacaggcgcctac

SEQ ID NO: 527 - DNA, 21 bp, Artificial Sequence
  Description of Artificial Sequence: Synthetic oligonucleotide
ggctacaccttcactacctac

SEQ ID NO: 528 - DNA, 18 bp, Artificial Sequence
  Description of Artificial Sequence: Synthetic oligonucleotide
taccccggcaccggcggc

SEQ ID NO: 529 - DNA, 51 bp, Artificial Sequence
  Description of Artificial Sequence: Synthetic oligonucleotide
aaatctagtcagtcactgctggatagcggtaatcagaagaacttcctgacc

SEQ ID NO: 530 - DNA, 21 bp, Artificial Sequence
  Description of Artificial Sequence: Synthetic oligonucleotide
tgggcctctactagagaatca

SEQ ID NO: 531 - DNA, 27 bp, Artificial Sequence
  Description of Artificial Sequence: Synthetic oligonucleotide
cagaacgactatagctacccctacacc

SEQ ID NO: 532 - DNA, 39 bp, Artificial Sequence
  Description of Artificial Sequence: Synthetic oligonucleotide
agtcagtcactgctggatagcggtaatcagaagaacttc

SEQ ID NO: 533 - DNA, 9 bp, Artificial Sequence
  Description of Artificial Sequence: Synthetic oligonucleotide
tgggcctct

SEQ ID NO: 534 - DNA, 18 bp, Artificial Sequence
  Description of Artificial Sequence: Synthetic oligonucleotide
gactatagctacccctac

SEQ ID NO: 535 - protein, 440 aa, Artificial Sequence
  Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, Artificial Sequence
  Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, Artificial Sequence
  Description of Artificial Sequence: Synthetic polypeptide
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, Artificial Sequence
  Description of Artificial Sequence: Synthetic polypeptide
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541-600 - skipped (60 entries)

SEQ ID NO: 601 - protein, 5 aa, Artificial Sequence
  Description of Artificial Sequence: Synthetic peptide
SYWMY

SEQ ID NO: 602 - protein, 17 aa, Artificial Sequence
  Description of Artificial Sequence: Synthetic peptide
RIDPNSGSTKYNEKFKN

SEQ ID NO: 603 - protein, 11 aa, Artificial Sequence
  Description of Artificial Sequence: Synthetic peptide
DYRKGLYAMDY

SEQ ID NO: 604 - protein, 7 aa, Artificial Sequence
  Description of Artificial Sequence: Synthetic peptide
GYTFTSY

SEQ ID NO: 605 - protein, 6 aa, Artificial Sequence
  Description of Artificial Sequence: Synthetic peptide
DPNSGS

SEQ ID NO: 606 - protein, 120 aa, Artificial Sequence
  Description of Artificial Sequence: Synthetic polypeptide
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607 - DNA, 360 bp, Artificial Sequence
  Description of Artificial Sequence: Synthetic polynucleotide
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 608 - protein, 446 aa, Artificial Sequence
  Description of Artificial Sequence: Synthetic polypeptide
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 609 - protein, 11 aa, Artificial Sequence
  Description of Artificial Sequence: Synthetic peptide
KASQDVGTAVA

SEQ ID NO: 610 - protein, 7 aa, Artificial Sequence
  Description of Artificial Sequence: Synthetic peptide
WASTRHT

SEQ ID NO: 611 - protein, 9 aa, Artificial Sequence
  Description of Artificial Sequence: Synthetic peptide
QQYNSYPLT

SEQ ID NO: 612 - protein, 7 aa, Artificial Sequence
  Description of Artificial Sequence: Synthetic peptide
SQDVGTA

SEQ ID NO: 613 - protein, 3 aa, Artificial Sequence
  Description of Artificial Sequence: Synthetic peptide
WAS

SEQ ID NO: 614 - protein, 6 aa, Artificial Sequence
  Description of Artificial Sequence: Synthetic peptide
YNSYPL

SEQ ID NO: 615 - DNA, 1338 bp, Artificial Sequence
  Description of Artificial Sequence: Synthetic polynucleotide
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 616 - protein, 107 aa, Artificial Sequence
  Description of Artificial Sequence: Synthetic polypeptide
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617 - DNA, 321 bp, Artificial Sequence
  Description of Artificial Sequence: Synthetic polynucleotide
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 618 - protein, 214 aa, Artificial Sequence
  Description of Artificial Sequence: Synthetic polypeptide
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 642 bp, Artificial Sequence
  Description of Artificial Sequence: Synthetic polynucleotide
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 620 - protein, 120 aa, Artificial Sequence
  Description of Artificial Sequence: Synthetic polypeptide
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621 - DNA, 360 bp, Artificial Sequence
  Description of Artificial Sequence: Synthetic polynucleotide
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 622 - protein, 446 aa, Artificial Sequence
  Description of Artificial Sequence: Synthetic polypeptide
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 623 - DNA, 1338 bp, Artificial Sequence
  Description of Artificial Sequence: Synthetic polynucleotide
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 624 - protein, 107 aa, Artificial Sequence
  Description of Artificial Sequence: Synthetic polypeptide
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625 - DNA, 321 bp, Artificial Sequence
  Description of Artificial Sequence: Synthetic polynucleotide
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 626 - protein, 214 aa, Artificial Sequence
  Description of Artificial Sequence: Synthetic polypeptide
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 642 bp, Artificial Sequence
  Description of Artificial Sequence: Synthetic polynucleotide
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 628 - DNA, 15 bp, Artificial Sequence
  Description of Artificial Sequence: Synthetic oligonucleotide
agctactggatgtac

SEQ ID NO: 629 - DNA, 51 bp, Artificial Sequence
  Description of Artificial Sequence: Synthetic oligonucleotide
agaatcgaccctaatagcggctctactaagtataacgagaagtttaagaat

SEQ ID NO: 630 - DNA, 33 bp, Artificial Sequence
  Description of Artificial Sequence: Synthetic oligonucleotide
gactatagaaagggcctgtacgctatggactac

SEQ ID NO: 631 - DNA, 21 bp, Artificial Sequence
  Description of Artificial Sequence: Synthetic oligonucleotide
ggctacaccttcactagctac

SEQ ID NO: 632 - DNA, 18 bp, Artificial Sequence
  Description of Artificial Sequence: Synthetic oligonucleotide
gaccctaatagcggctct

SEQ ID NO: 633 - DNA, 33 bp, Artificial Sequence
  Description of Artificial Sequence: Synthetic oligonucleotide
aaagcctctcaggacgtgggcaccgccgtggcc

SEQ ID NO: 634 - DNA, 21 bp, Artificial Sequence
  Description of Artificial Sequence: Synthetic oligonucleotide
tgggcctctactagacacacc

SEQ ID NO: 635 - DNA, 27 bp, Artificial Sequence
  Description of Artificial Sequence: Synthetic oligonucleotide
cagcagtataatagctaccccctgacc

SEQ ID NO: 636 - DNA, 21 bp, Artificial Sequence
  Description of Artificial Sequence: Synthetic oligonucleotide
tctcaggacgtgggcaccgcc

SEQ ID NO: 637 - DNA, 9 bp, Artificial Sequence
  Description of Artificial Sequence: Synthetic oligonucleotide
tgggcctct

SEQ ID NO: 638 - DNA, 18 bp, Artificial Sequence
  Description of Artificial Sequence: Synthetic oligonucleotide
tataatagctaccccctg

SEQ ID NO: 639 - protein, 448 aa, Artificial Sequence
  Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 214 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 450 aa, Artificial Sequence
  Description of Artificial Sequence: Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 216 aa, Artificial Sequence
  Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 643 - protein, 451 aa, Artificial Sequence
  Description of Artificial Sequence: Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 215 aa, Artificial Sequence
  Description of Artificial Sequence: Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - protein, 123 aa, Artificial Sequence
  Description of Artificial Sequence: Synthetic polypeptide
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 646 - protein, 106 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 647 - protein, 10 aa, Artificial Sequence
  Description of Artificial Sequence: Synthetic peptide
GYTFTSYWMY

SEQ ID NO: 648-700 - skipped (53 entries)

SEQ ID NO: 701 - protein, 5 aa, Artificial Sequence
  Description of Artificial Sequence: Synthetic peptide
NYGMN

SEQ ID NO: 702 - protein, 17 aa, Artificial Sequence
  Description of Artificial Sequence: Synthetic peptide
WINTDTGEPTYADDFKG

SEQ ID NO: 703 - protein, 16 aa, Artificial Sequence
  Description of Artificial Sequence: Synthetic peptide
NPPYYYGTNNAEAMDY

SEQ ID NO: 704 - protein, 7 aa, Artificial Sequence
  Description of Artificial Sequence: Synthetic peptide
GFTLTNY

SEQ ID NO: 705 - protein, 6 aa, Artificial Sequence
  Description of Artificial Sequence: Synthetic peptide
NTDTGE

SEQ ID NO: 706 - protein, 125 aa, Artificial Sequence
  Description of Artificial Sequence: Synthetic polypeptide
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707 - DNA, 375 bp, Artificial Sequence
  Description of Artificial Sequence: Synthetic polynucleotide
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 708 - DNA, 375 bp, Artificial Sequence
  Description of Artificial Sequence: Synthetic polynucleotide
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 709 - protein, 451 aa, Artificial Sequence
  Description of Artificial Sequence: Synthetic polypeptide
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 710 - protein, 11 aa, Artificial Sequence
  Description of Artificial Sequence: Synthetic peptide
SSSQDISNYLN

SEQ ID NO: 711 - protein, 7 aa, Artificial Sequence
  Description of Artificial Sequence: Synthetic peptide
YTSTLHL

SEQ ID NO: 712 - protein, 9 aa, Artificial Sequence
  Description of Artificial Sequence: Synthetic peptide
QQYYNLPWT

SEQ ID NO: 713 - protein, 7 aa, Artificial Sequence
  Description of Artificial Sequence: Synthetic peptide
SQDISNY

SEQ ID NO: 714 - protein, 3 aa, Artificial Sequence
  Description of Artificial Sequence: Synthetic peptide
YTS

SEQ ID NO: 715 - protein, 6 aa, Artificial Sequence
  Description of Artificial Sequence: Synthetic peptide
YYNLPW

SEQ ID NO: 716 - DNA, 1353 bp, Artificial Sequence
  Description of Artificial Sequence: Synthetic polynucleotide
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 717 - DNA, 1353 bp, Artificial Sequence
  Description of Artificial Sequence: Synthetic polynucleotide
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 718 - protein, 107 aa, Artificial Sequence
  Description of Artificial Sequence: Synthetic polypeptide
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719 - DNA, 321 bp, Artificial Sequence
  Description of Artificial Sequence: Synthetic polynucleotide
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 720 - DNA, 321 bp, Artificial Sequence
  Description of Artificial Sequence: Synthetic polynucleotide
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 721 - protein, 214 aa, Artificial Sequence
  Description of Artificial Sequence: Synthetic polypeptide
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - DNA, 642 bp, Artificial Sequence
  Description of Artificial Sequence: Synthetic polynucleotide
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 723 - DNA, 642 bp, Artificial Sequence
  Description of Artificial Sequence: Synthetic polynucleotide
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 724 - protein, 125 aa, Artificial Sequence
  Description of Artificial Sequence: Synthetic polypeptide
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725 - DNA, 375 bp, Artificial Sequence
  Description of Artificial Sequence: Synthetic polynucleotide
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 726 - DNA, 375 bp, Artificial Sequence
  Description of Artificial Sequence: Synthetic polynucleotide
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 727 - protein, 451 aa, Artificial Sequence
  Description of Artificial Sequence: Synthetic polypeptide
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 728 - DNA, 1353 bp, Artificial Sequence
  Description of Artificial Sequence: Synthetic polynucleotide
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 729 - DNA, 1353 bp, Artificial Sequence
  Description of Artificial Sequence: Synthetic polynucleotide
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 730 - protein, 107 aa, Artificial Sequence
  Description of Artificial Sequence: Synthetic polypeptide
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731 - DNA, 321 bp, Artificial Sequence
  Description of Artificial Sequence: Synthetic polynucleotide
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 732 - DNA, 321 bp, Artificial Sequence
  Description of Artificial Sequence: Synthetic polynucleotide
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 733 - protein, 214 aa, Artificial Sequence
  Description of Artificial Sequence: Synthetic polypeptide
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - DNA, 642 bp, Artificial Sequence
  Description of Artificial Sequence: Synthetic polynucleotide
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 735 - DNA, 642 bp, Artificial Sequence
  Description of Artificial Sequence: Synthetic polynucleotide
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 736 - DNA, 15 bp, Artificial Sequence
  Description of Artificial Sequence: Synthetic oligonucleotide
aattacgggatgaac

SEQ ID NO: 737 - DNA, 15 bp, Artificial Sequence
  Description of Artificial Sequence: Synthetic oligonucleotide
aactacggcatgaac

SEQ ID NO: 738 - DNA, 51 bp, Artificial Sequence
  Description of Artificial Sequence: Synthetic oligonucleotide
tggattaacaccgacaccggggagcctacctacgcggacgatttcaaggga

SEQ ID NO: 739 - DNA, 51 bp, Artificial Sequence
  Description of Artificial Sequence: Synthetic oligonucleotide
tggatcaacaccgacaccggcgagcctacctacgccgacgacttcaagggc

SEQ ID NO: 740 - DNA, 48 bp, Artificial Sequence
  Description of Artificial Sequence: Synthetic oligonucleotide
aacccgccctactactacggaaccaacaacgccgaagccatggactac

SEQ ID NO: 741 - DNA, 48 bp, Artificial Sequence
  Description of Artificial Sequence: Synthetic oligonucleotide
aaccccccttactactacggcaccaacaacgccgaggccatggactat

SEQ ID NO: 742 - DNA, 21 bp, Artificial Sequence
  Description of Artificial Sequence: Synthetic oligonucleotide
ggattcaccctcaccaattac

SEQ ID NO: 743 - DNA, 21 bp, Artificial Sequence
  Description of Artificial Sequence: Synthetic oligonucleotide
ggcttcaccctgaccaactac

SEQ ID NO: 744 - DNA, 18 bp, Artificial Sequence
  Description of Artificial Sequence: Synthetic oligonucleotide
aacaccgacaccggggag

SEQ ID NO: 745 - DNA, 18 bp, Artificial Sequence
  Description of Artificial Sequence: Synthetic oligonucleotide
aacaccgacaccggcgag

SEQ ID NO: 746 - DNA, 33 bp, Artificial Sequence
  Description of Artificial Sequence: Synthetic oligonucleotide
agctctagtcaggatatctctaactacctgaac

SEQ ID NO: 747 - DNA, 33 bp, Artificial Sequence
  Description of Artificial Sequence: Synthetic oligonucleotide
tcctccagccaggacatctccaactacctgaac

SEQ ID NO: 748 - DNA, 21 bp, Artificial Sequence
  Description of Artificial Sequence: Synthetic oligonucleotide
tacactagcaccctgcacctg

SEQ ID NO: 749 - DNA, 21 bp, Artificial Sequence
  Description of Artificial Sequence: Synthetic oligonucleotide
tacacctccaccctgcacctg

SEQ ID NO: 750 - DNA, 27 bp, Artificial Sequence
  Description of Artificial Sequence: Synthetic oligonucleotide
cagcagtactataacctgccctggacc

SEQ ID NO: 751 - DNA, 27 bp, Artificial Sequence
  Description of Artificial Sequence: Synthetic oligonucleotide
cagcagtactacaacctgccctggacc

SEQ ID NO: 752 - DNA, 21 bp, Artificial Sequence
  Description of Artificial Sequence: Synthetic oligonucleotide
agtcaggatatctctaactac

SEQ ID NO: 753 - DNA, 21 bp, Artificial Sequence
  Description of Artificial Sequence: Synthetic oligonucleotide
agccaggacatctccaactac

SEQ ID NO: 754 - DNA, 9 bp, Artificial Sequence
  Description of Artificial Sequence: Synthetic oligonucleotide
tacactagc

SEQ ID NO: 755 - DNA, 9 bp, Artificial Sequence
  Description of Artificial Sequence: Synthetic oligonucleotide
tacacctcc

SEQ ID NO: 756 - DNA, 18 bp, Artificial Sequence
  Description of Artificial Sequence: Synthetic oligonucleotide
tactataacctgccctgg

SEQ ID NO: 757 - DNA, 18 bp, Artificial Sequence
  Description of Artificial Sequence: Synthetic oligonucleotide
tactacaacctgccctgg

SEQ ID NO: 758 - DNA, 15 bp, Artificial Sequence
  Description of Artificial Sequence: Synthetic oligonucleotide
aactacgggatgaac

SEQ ID NO: 759 - DNA, 51 bp, Artificial Sequence
  Description of Artificial Sequence: Synthetic oligonucleotide
tggattaacaccgacaccggcgagcctacctacgccgacgactttaagggc

SEQ ID NO: 760 - DNA, 48 bp, Artificial Sequence
  Description of Artificial Sequence: Synthetic oligonucleotide
aaccccccctactactacggcactaacaacgccgaggctatggactac

SEQ ID NO: 761 - DNA, 21 bp, Artificial Sequence
  Description of Artificial Sequence: Synthetic oligonucleotide
ggcttcaccctgactaactac

SEQ ID NO: 762 - protein, 447 aa, Artificial Sequence
  Description of Artificial Sequence: Synthetic polypeptide
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, Artificial Sequence
  Description of Artificial Sequence: Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, Artificial Sequence
  Description of Artificial Sequence: Synthetic polypeptide
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, Artificial Sequence
  Description of Artificial Sequence: Synthetic polypeptide
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766-800 - skipped (35 entries)

SEQ ID NO: 801 - protein, 5 aa, Artificial Sequence
  Description of Artificial Sequence: Synthetic peptide
SYNMH

SEQ ID NO: 802 - protein, 17 aa, Artificial Sequence
  Description of Artificial Sequence: Synthetic peptide
DIYPGNGDTSYNQKFKG

SEQ ID NO: 803 - protein, 9 aa, Artificial Sequence
  Description of Artificial Sequence: Synthetic peptide
VGGAFPMDY

SEQ ID NO: 804 - protein, 7 aa, Artificial Sequence
  Description of Artificial Sequence: Synthetic peptide
GYTFTSY

SEQ ID NO: 805 - protein, 6 aa, Artificial Sequence
  Description of Artificial Sequence: Synthetic peptide
YPGNGD

SEQ ID NO: 806 - protein, 118 aa, Artificial Sequence
  Description of Artificial Sequence: Synthetic polypeptide
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, Artificial Sequence
  Description of Artificial Sequence: Synthetic polynucleotide
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, Artificial Sequence
  Description of Artificial Sequence: Synthetic polypeptide
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, Artificial Sequence
  Description of Artificial Sequence: Synthetic polynucleotide
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, Artificial Sequence
  Description of Artificial Sequence: Synthetic peptide
RASESVEYYGTSLMQ

SEQ ID NO: 811 - protein, 7 aa, Artificial Sequence
  Description of Artificial Sequence: Synthetic peptide
AASNVES

SEQ ID NO: 812 - protein, 9 aa, Artificial Sequence
  Description of Artificial Sequence: Synthetic peptide
QQSRKDPST

SEQ ID NO: 813 - protein, 11 aa, Artificial Sequence
  Description of Artificial Sequence: Synthetic peptide
SESVEYYGTSL

SEQ ID NO: 814 - protein, 3 aa, Artificial Sequence
  Description of Artificial Sequence: Synthetic peptide
AAS

SEQ ID NO: 815 - protein, 6 aa, Artificial Sequence
  Description of Artificial Sequence: Synthetic peptide
SRKDPS

SEQ ID NO: 816 - protein, 111 aa, Artificial Sequence
  Description of Artificial Sequence: Synthetic polypeptide
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, Artificial Sequence
  Description of Artificial Sequence: Synthetic polynucleotide
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, Artificial Sequence
  Description of Artificial Sequence: Synthetic polypeptide
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, Artificial Sequence
  Description of Artificial Sequence: Synthetic polynucleotide
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, Artificial Sequence
  Description of Artificial Sequence: Synthetic peptide
DIYPGQGDTSYNQKFKG

SEQ ID NO: 821 - protein, 6 aa, Artificial Sequence
  Description of Artificial Sequence: Synthetic peptide
YPGQGD

SEQ ID NO: 822 - protein, 118 aa, Artificial Sequence
  Description of Artificial Sequence: Synthetic polypeptide
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, Artificial Sequence
  Description of Artificial Sequence: Synthetic polynucleotide
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, Artificial Sequence
  Description of Artificial Sequence: Synthetic polypeptide
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, Artificial Sequence
  Description of Artificial Sequence: Synthetic polynucleotide
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, Artificial Sequence
  Description of Artificial Sequence: Synthetic polypeptide
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, Artificial Sequence
  Description of Artificial Sequence: Synthetic polynucleotide
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, Artificial Sequence
  Description of Artificial Sequence: Synthetic polypeptide
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, Artificial Sequence
  Description of Artificial Sequence: Synthetic polynucleotide
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, Artificial Sequence
  Description of Artificial Sequence: Synthetic polypeptide
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  Description of Artificial Sequence: Synthetic polypeptide
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  Description of Artificial Sequence: Synthetic polypeptide
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  Description of Artificial Sequence: Synthetic polypeptide
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, Artificial Sequence
  Description of Artificial Sequence: Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, Artificial Sequence
  Description of Artificial Sequence: Synthetic polypeptide
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903 - protein, 451 aa, Artificial Sequence
  Description of Artificial Sequence: Synthetic polypeptide
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 904 - protein, 214 aa, Artificial Sequence
  Description of Artificial Sequence: Synthetic polypeptide
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 905 - DNA, 363 bp, Artificial Sequence
  Description of Artificial Sequence: Synthetic polynucleotide
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 906 - DNA, 321 bp, Artificial Sequence
  Description of Artificial Sequence: Synthetic polynucleotide
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 907 - DNA, 1353 bp, Artificial Sequence
  Description of Artificial Sequence: Synthetic polynucleotide
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 908 - DNA, 642 bp, Artificial Sequence
  Description of Artificial Sequence: Synthetic polynucleotide
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 909 - protein, 5 aa, Artificial Sequence
  Description of Artificial Sequence: Synthetic peptide
SYGVD

SEQ ID NO: 910 - protein, 7 aa, Artificial Sequence
  Description of Artificial Sequence: Synthetic peptide
GFSLSSY

SEQ ID NO: 911 - protein, 16 aa, Artificial Sequence
  Description of Artificial Sequence: Synthetic peptide
VIWGGGGTYYASSLMG

SEQ ID NO: 912 - protein, 5 aa, Artificial Sequence
  Description of Artificial Sequence: Synthetic peptide
WGGGG

SEQ ID NO: 913 - protein, 13 aa, Artificial Sequence
  Description of Artificial Sequence: Synthetic peptide
HAYGHDGGFAMDY

SEQ ID NO: 914 - protein, 11 aa, Artificial Sequence
  Description of Artificial Sequence: Synthetic peptide
RASESVSSNVA

SEQ ID NO: 915 - protein, 7 aa, Artificial Sequence
  Description of Artificial Sequence: Synthetic peptide
SESVSSN

SEQ ID NO: 916 - protein, 7 aa, Artificial Sequence
  Description of Artificial Sequence: Synthetic peptide
GASNRAT

SEQ ID NO: 917 - protein, 3 aa, Artificial Sequence
  Description of Artificial Sequence: Synthetic peptide
GAS

SEQ ID NO: 918 - protein, 9 aa, Artificial Sequence
  Description of Artificial Sequence: Synthetic peptide
GQSYSYPFT

SEQ ID NO: 919 - protein, 6 aa, Artificial Sequence
  Description of Artificial Sequence: Synthetic peptide
SYSYPF

SEQ ID NO: 920 - protein, 124 aa, Artificial Sequence
  Description of Artificial Sequence: Synthetic polypeptide
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, Artificial Sequence
  Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 923 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 924 - protein, 114 aa, Artificial Sequence
  Description of Artificial Sequence: Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 925 - protein, 297 aa, Artificial Sequence
  Description of Artificial Sequence: Synthetic polypeptide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 926 - protein, 114 aa, Artificial Sequence
  Description of Artificial Sequence: Synthetic polypeptide
  MOD_RES (93)..(93): Glu or Lys
NWVNVISDLKKIEDLIQSMHIDATLYTESDVHPSCKVTAMKCFLLELQVISLESGDASIHDTVENLIILANNSLSSNGNVTESGCKECEELEXKNIKEFLQSFVHIVQMFINTS

SEQ ID NO: 927 - protein, 77 aa, Artificial Sequence
  Description of Artificial Sequence: Synthetic polypeptide
ITCPPPMSVEHADIWVKSYSLYSRERYICNSGFKRKAGTSSLTECVLNKATNVAHWTTPSLKCIRDPALVHQRPAPP

SEQ ID NO: 928 - skipped

SEQ ID NO: 929 - protein, 22 aa, Artificial Sequence
  Description of Artificial Sequence: Synthetic peptide
YGRKKRRQRRRLYRSPAMPENL

SEQ ID NO: 930 - protein, 6 aa, Artificial Sequence
  Description of Artificial Sequence: Synthetic 6xHis tag
HHHHHH

SEQ ID NO: 931 - DNA, 20 bp, Artificial Sequence
  Description of Artificial Sequence: Synthetic oligonucleotide
tccatccatcccgtgtccca

SEQ ID NO: 932 - DNA, 2223 bp, Homo sapiens
tataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagattacccaaaaaaaaaaaaaaaaaa

SEQ ID NO: 933 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 934 - DNA, 1795 bp, Homo sapiens
gctgcactcagagaagctgcccttggctgctcgtagcgccgggccttctctcctcgtcatcatccagagcagccagtgtccgggaggcagaagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagattacccaaaaaaaaaaaaaaaaaa

SEQ ID NO: 935 - protein, 283 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRNLQMTAASRCPRRFSGTCGRRKRKRLLWAA

SEQ ID NO: 936 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 937 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 938 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccgctgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 939 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcaccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccgctgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 940 - protein, 368 aa, Artificial Sequence
  Description of Artificial Sequence: Synthetic polypeptide
GPDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 941 - protein, 362 aa, Mus sp.
GPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL